(19) United States
(12) Patent Application Publication
(10) Pub. No.: US 2024/0401047 A1
(43) Pub. Date: Dec. 5, 2024

(54) TREM COMPOSITIONS AND METHODS OF USE

(71) Applicant: Flagship Pioneering Innovations VI, LLC, Cambridge, MA (US)

(72) Inventors: Theonie Anastassiadis, Somerville, MA (US); Neil Kubica, Swampscott, MA (US); Qingyi Li, Somerville, MA (US); Nikolai Naryshkin, East Brunswick, NJ (US); Stephen William Eichhorn, Medford, MA (US); William F. Kiesman, Wayland, MA (US); Christopher E. Arcadia, Barrington, RI (US); Hongchuan Yu, Chestnut Hill, MA (US)

(21) Appl. No.: 18/700,523

(22) PCT Filed: Oct. 13, 2022
(86) PCT No.: PCT/US2022/046632

§ 371 Date: Apr. 11, 2024

Related U.S. Application Data
(60) Provisional application No. 63/284,934, filed on Dec. 1, 2021.
(60) Provisional application No. 63/284,946, filed on Dec. 1, 2021.
(60) Provisional application No. 63/255,420, filed on Oct. 13, 2021.
(60) Provisional application No. 63/255,426, filed on Oct. 13, 2021.

(51) Int. Cl. C12N 15/113; A61K 9/51

(52) U.S. Cl. CPC C12N 15/113; A61K 9/5123; C12N 2310/315; C12N 2310/321; C12N 2310/322; C12N 2310/531

(57) ABSTRACT

The invention relates generally to tRNA-based effector molecules having a non-naturally occurring modification and methods relating thereto.

### CROSS-REFERENCE TO RELATED APPLICATIONS

This application claims priority to U.S. Provisional Application No. 63/255,420, filed on Oct. 13, 2021; U.S. Provisional Application No. 63/255,426, filed on Oct. 13, 2021; U.S. Provisional Application No. 63/284,934, filed on Dec. 1, 2021; and U.S. Provisional Application No. 63/284,946, filed on Dec. 1, 2021; the entire contents of each of the foregoing applications is hereby incorporated by reference.

### BACKGROUND

Transfer RNAs (tRNAs) are complex, naturally occurring RNA molecules that possess a number of functions including initiation and elongation of proteins.

### SUMMARY

The present disclosure features modified tRNA-based effector molecules (TREMs, e.g., a TREM or TREM fragment), as well as related compositions and uses thereof. As provided herein, TREMs are complex molecules which can mediate a variety of cellular processes. The TREMs disclosed herein comprise at least one modification (e.g., a non-naturally occurring modification), e.g., on a component nucleotide (e.g., a nucleobase or sugar) or within an internucleotide region, e.g., the TREM backbone. In one aspect, provided herein is a TREM comprising a sequence of Formula A: [L1]-[ASt Domain1]-[L2]-[DH Domain]-[L3]-[ACH Domain]-[VL Domain]-[TH Domain]-[L4]-[ASt Domain2], wherein independently, [L1] and [VL Domain], are optional; and one of [L1], [ASt Domain1], [L2]-[DH Domain], [L3], [ACH Domain], [VL Domain], [TH Domain], [L4], and [ASt Domain2] comprises a nucleotide comprising a non-naturally occurring modification.

In an embodiment, the TREM: (a) has the ability to: (i) support protein synthesis, (ii) be charged by a synthetase, (iii) be bound by an elongation factor, (iv) introduce an amino acid into a peptide chain, (v) support elongation, or (vi) support initiation; (b) comprises at least X contiguous nucleotides without a non-naturally occurring modification, wherein X is greater than 3, 4, 5, 6, 7, 8, 9, or 10; (c) comprises at least 3, but less than all of the nucleotides of a type (e.g., A. T. C. G or U) comprise the same non-naturally occurring modification; (d) comprises at least X nucleotides of a type (e.g., A. T. C. G or U) that do not comprise a non-naturally occurring modification, wherein X=than 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, or 80; (e) comprises no more than 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, or 80 nucleotides of a type (e.g., A. T. C. G or U) that comprise a non-naturally occurring modification; and/or (f) comprises no more than 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, or 80 nucleotides of a type (e.g., A. T. C. G or U) that do not comprise a non-naturally occurring modification.

In an embodiment, the TREM comprises feature (a) (i). In an embodiment, the TREM comprises feature (a) (ii). In an embodiment, the TREM comprises feature (a) (iii). In an embodiment, the TREM comprises feature (a) (iv). In an embodiment, the TREM comprises feature (a) (v). In an embodiment, the TREM comprises feature (a) (vi). In an embodiment, the TREM comprises feature (b). In an embodiment, the TREM comprises feature (c). In an embodiment, the TREM comprises feature (d). In an embodiment, the TREM comprises feature (e). In an embodiment, the TREM comprises feature (f). In an embodiment, the TREM comprises all of features (a)-(f) or a combination thereof.

In an embodiment, the TREM Domain comprising the non-naturally occurring modification has a function, e.g., a domain function described herein.

In an aspect, provided herein is a TREM core fragment comprising a sequence of Formula B:
[L1]_{y}-[ASt Domain1]ₓ-[L2]_{y}-[DH Domain]_{y}-[L3]_{y}-[ACH Domain]ₓ-[VL Domain]_{y}-[TH Domain]_{y}-[L4]_{y}-[ASt Domain2]ₓ,
wherein x=1 and y=0 or 1; and one of [ASt Domain1], [ACH Domain], and [ASt Domain2] comprises a nucleotide having a non-naturally occurring modification.

In an embodiment, the TREM has the ability to support protein synthesis. In an embodiment, the TREM has the ability to be able to be charged by a synthetase. In an embodiment, the TREM has the ability to be bound by an elongation factor. In an embodiment, the TREM has the ability to introduce an amino acid into a peptide chain. In an embodiment, the TREM has the ability to support elongation. In an embodiment, the TREM has the ability to support initiation.

In an embodiment, the [ASt Domain 1] and/or [ASt Domain 2] comprising the non-naturally occurring modification has the ability to initiate or elongate a polypeptide chain.

In an embodiment, the [ACH Domain] comprising the non-naturally occurring modification has the ability to mediate pairing with a codon.

In an embodiment, y=1 for any one, two, three, four, five, six, all or a combination of [L1], [L2], [DH Domain], [L3], [VL Domain], [TH Domain], [L4].

In an embodiment, y=0 for any one, two, three, four, five, six, all or a combination of [L1], [L2], [DH Domain], [L3], [VL Domain], [TH Domain], [L4].

In an embodiment, y=1 for linker [L1], and L1 comprises a nucleotide having a non-naturally occurring modification.

In an embodiment, y=1 for linker [L2], and L2 comprises a nucleotide having a non-naturally occurring modification.

In an embodiment, y=1 for [DH Domain (DHD)], and DHD comprises a nucleotide having a non-naturally occurring modification. In an embodiment, the DHD comprising the non-naturally occurring modification has the ability to mediate recognition of aminoacyl-tRNA synthetase.

In an embodiment, y=1 for linker [L3], and L3 comprises a nucleotide having a non-naturally occurring modification.

In an embodiment, y=1 for [VL Domain (VLD)], and VLD comprises a nucleotide having a non-naturally occurring modification.

In an embodiment, y=1 for [TH Domain (THD)], and THD comprises a nucleotide having a non-naturally occurring modification. In an embodiment, the THD comprising the non-naturally occurring modification has the ability to mediate recognition of the ribosome.

In an embodiment, y=1 for linker [L4], and L4 comprises a nucleotide having a non-naturally occurring modification.

In another aspect, the disclosure provides a TREM fragment comprising a portion of a TREM, wherein the TREM comprises a sequence of Formula A:
[L1]-[ASt Domain1]-[L2]-[DH Domain]-[L3]-[ACH Domain]-[VL Domain]-[TH Domain]-[L4]-[ASt Domain2], and wherein the TREM fragment comprises a non-naturally occurring modification.

In an embodiment, the TREM fragment comprises one, two, three or all or any combination of the following: (a) a TREM half (e.g., from a cleavage in the ACH Domain, e.g., in the anticodon sequence, e.g., a 5′half or a 3′ half); (b) a 5′ fragment (e.g., a fragment comprising the 5′ end, e.g., from a cleavage in a DH Domain or the ACH Domain); (c) a 3′ fragment (e.g., a fragment comprising the 3′ end, e.g., from a cleavage in the TH Domain); or (d) an internal fragment (e.g., from a cleavage in any one of the ACH Domain, DH Domain or TH Domain).

In an embodiment, the TREM fragment comprise (a) a TREM half which comprises a nucleotide having a non-naturally occurring modification.

In an embodiment, the TREM fragment comprise (b) a 5′ fragment which comprises a nucleotide having a non-naturally occurring modification.

In an embodiment, the TREM fragment comprise (c) a 3′ fragment which comprises a nucleotide having a non-naturally occurring modification.

In an embodiment, the TREM fragment comprise (d) an internal fragment which comprises a nucleotide having a non-naturally occurring modification.

In an embodiment of any of the TREMs, TREM core fragments, or TREM fragments disclosed herein, the TREM Domain comprises a plurality of nucleotides each having a non-naturally occurring modification. In an embodiment, the non-naturally occurring modification comprises a nucleobase modification, a sugar (e.g., ribose) modification, or a backbone modification. In an embodiment, tbe non-naturally occurring modification is a sugar (e.g., ribose) modification. In an embodiment, tbe non-naturally occurring modification is 2′-ribose modification, e.g., a 2′-OMe, 2′-halo (e.g., 2′-F), 2′-MOE, or 2′-deoxy modification. In an embodiment, tbe non-naturally occurring modification is a backbone modification, e.g., a phosphorothioate modification.

In an embodiment of any of the TREMs, TREM core fragments, or TREM fragments disclosed herein, the TREM sequence comprises a CCA sequence on a terminus, e.g., the 3′ terminus. In an embodiment, the TREM sequence does not comprise a CCA sequence on a terminus, e.g., the 3′ terminus.

In an embodiment of any of the TREMs, TREM core fragments, or TREM fragments disclosed herein, the non-naturally occurring modification is a modification in a base or a backbone of a nucleotide, e.g., a modification chosen from any one of Tables 5, 6, 7, 8 or 9.

In an embodiment of any of the TREMs, TREM core fragments, or TREM fragments disclosed herein, the non-naturally occurring modification is a base modification chosen from a modification listed in Table 5.

In an embodiment of any of the TREMs, TREM core fragments, or TREM fragments disclosed herein, the non-naturally occurring modification is a base modification chosen from a modification listed in Table 6.

In an embodiment of any of the TREMs, TREM core fragments, or TREM fragments disclosed herein, the non-naturally occurring modification is a base modification chosen from a modification listed in Table 7.

In an embodiment of any of the TREMs, TREM core fragments, or TREM fragments disclosed herein, the non-naturally occurring modification is a backbone modification chosen from a modification listed in Table 8.

In an embodiment of any of the TREMs, TREM core fragments, or TREM fragments disclosed herein, the non-naturally occurring modification is a backbone modification chosen from a modification listed in Table 9.

In an embodiment of any of the TREMs, TREM core fragments, or TREM fragments disclosed herein, the TREM, TREM core fragment, or TREM fragment is encoded by a sequence provided in Table 1, e.g., any one of SEQ ID NOs 1-451.

In an embodiment of any of the TREMs, TREM core fragments, or TREM fragments disclosed herein, the TREM, TREM core fragment, or TREM fragment is encoded by a consensus sequence chosen from any one of SEQ ID NOs: 562-621.

In an embodiment of any of the TREMs, TREM core fragments, or TREM fragments disclosed herein, the TREM, TREM core fragment, or TREM fragment is encoded by a sequence provided in FIG. 2, e.g., any one of SEQ ID NOs: 622-9757. In an embodiment, the TREM, TREM core fragment, or TREM fragment comprises a TREM having at least 70%, 75%, 80%, 85%, 90%, 95%, or 99% sequence identity with a TREM provided in FIG. 2, e.g., any one of SEQ ID NOs: 622-9757. In an embodiment, the TREM, TREM core fragment, or TREM fragment comprises a sequence that differs by no more than 1, 2, 3, 4, 5, 6, 7, 8, 9, or 10 nucleotides from a TREM provided in FIG. 2, e.g., any one of SEQ ID NOs: 622-3284. In an embodiment, the TREM, TREM core fragment, or TREM fragment comprises 1, 2, 3, 4, 5, 6, 7, 8, 9, or 10 additional non-naturally occurring modifications compared with a TREM, TREM core fragment, or TREM fragment provided in FIG. 2 (e.g., 2′-ribose modifications or an internucleotide modification, e.g., 2′OMe, 2′-halo, 2′-MOE, 2′-deoxy, or phosphorothiorate modifications), e.g., any one of SEQ ID NOs: 622-9757.

In an embodiment of any of the TREMs, TREM core fragments, or TREM fragments disclosed herein, the TREM, TREM core fragment, or TREM fragment is a TREM provided in FIG. 2, e.g., any one of TREM NOs: 1-9757. In an embodiment, the TREM, TREM core fragment, or TREM fragment comprises 1, 2, 3, 4, 5, 6, 7, 8, 9, or 10 additional non-naturally occurring modifications compared with a TREM provided in FIG. 2 (e.g., 2′-ribose modifications or an internucleotide modification, e.g., 2′OMe, 2′-halo, 2′-MOE, 2′-deoxy, or phosphorothiorate modifications), e.g., any one of TREM NOs. 1-2663.

In another aspect, the disclosure provides a pharmaceutical composition comprising a TREM, a TREM core fragment, or a TREM fragment disclosed herein.

In another aspect, a TREM or a related composition thereof can be used, inter alia, to modulate a production parameter (e.g., an expression parameter and/or a signaling parameter) of an RNA corresponding to, or a polypeptide encoded by, a nucleic acid sequence comprising an endogenous open reading frame (ORF) having a premature termination codon (PTC).

In another aspect, provided herein is a method of modulating a production parameter of an mRNA corresponding to, or polypeptide encoded by, an endogenous open reading frame (ORF) in a subject, which ORF comprises a premature termination codon (PTC), contacting the subject with a TREM composition comprising a TREM, a TREM core fragment, or a TREM fragment disclosed herein in an amount and/or for a time sufficient to modulate the production parameter of the mRNA or polypeptide, wherein the TREM, TREM core fragment or TREM fragment has an anticodon that pairs with the codon having the first sequence, thereby modulating the production parameter in the subject. In an embodiment, the production parameter comprises a signaling parameter and/or an expression parameter, e.g., as described herein.

In another aspect, disclosed herein is a method of modulating expression of a protein in a cell, wherein the protein is encoded by a nucleic acid comprising an endogenous open reading frame (ORF), which ORF comprises a premature termination codon (PTC), comprising contacting the cell with a TREM composition comprising a TREM, a TREM core fragment, or a TREM fragment disclosed herein in an amount and/or for a time sufficient to modulate expression of the encoded protein, wherein the TREM, TREM core fragment or TREM fragment has an anticodon that pairs with the PTC, thereby modulating expression of the protein in the cell. In an embodiment, the PTC comprises UAA, UGA or UAG.

In another aspect, provided herein is a method of increasing expression of a protein in a subject wherein the protein is encoded by a nucleic acid comprising an endogenous open reading frame (ORF), which ORF comprises a premature termination codon (PTC), comprising contacting the subject, in an amount and/or for a time sufficient to increase expression of the protein, with a TREM composition that (i) has an anticodon that pairs with the PTC, (ii) recognizes an aminoacyl-tRNA synthetase specific for Trp, Tyr, Cys, Glu, Lys, Gln, Ser, Leu, Arg, or Gly, (iii) comprises a sequence of Formula A, or (iv) comprises a non-naturally occurring modification. In an embodiment, the PTC comprises UAA, UGA or UAG. In an embodiment, the TREM composition comprises (i). In an embodiment, the TREM composition comprises (ii).

In an embodiment, the TREM composition comprises (iii). In an embodiment, the TREM composition comprises (iv). In an embodiment, the TREM composition comprises two of (i)-(iv). In an embodiment, the TREM composition comprises three of (i)-(iv). In an embodiment, the TREM composition comprises each of (i)-(iv).

In another aspect, the disclosure provides a method of treating a subject having an endogenous open reading frame (ORF) which comprises a premature termination codon (PTC), comprising providing a TREM composition comprising a TREM, a TREM core fragment, or a TREM fragment disclosed herein, wherein the TREM comprises an anticodon that pairs with the PTC in the ORF; contacting the subject with the composition comprising a TREM, TREM core fragment or TREM fragment in an amount and/or for a time sufficient to treat the subject, thereby treating the subject. In an embodiment, the PTC comprises UAA, UGA or UAG.

In another aspect, the disclosure provides a method of treating a subject having an disease or disorder associated with a premature termination codon (PTC), comprising providing a TREM composition comprising a TREM, a TREM core fragment, or a TREM fragment disclosed herein; contacting the subject with the composition comprising a TREM, TREM core fragment or TREM fragment in an amount and/or for a time sufficient to treat the subject, thereby treating the subject. In an embodiment, the PTC comprises UAA, UGA or UAG. In an embodiment, the disease or disorder associated with a PTC is a disease or disorder described herein, e.g., a cancer or a monogenic disease.

In an embodiment of any of the methods disclosed herein, the codon having the first sequence comprises a mutation (e.g., a point mutation, e.g., a nonsense mutation), resulting in a premature termination codon (PTC) chosen from UAA, UGA or UAG. In an embodiment, the codon having the first sequence or the PTC comprises a UAA mutation. In an embodiment, the codon having the first sequence or the PTC comprises a UGA mutation. In an embodiment, the codon having the first sequence or the PTC comprises a UAG mutation

In another aspect, the disclosure provides a method of making a TREM, a TREM core fragment, or a TREM fragment disclosed herein, comprising linking a first nucleotide to a second nucleotide to form the TREM.

In an embodiment, the TREM, TREM core fragment or TREM fragment is non-naturally occurring (e.g., synthetic).

In an embodiment, the TREM, TREM core fragment or TREM fragment is made by cell-free solid phase synthesis.

In another aspect, the disclosure provides a method of modulating a tRNA pool in a cell comprising: providing a TREM, a TREM core fragment, or a TREM fragment disclosed herein, and contacting the cell with the TREM, TREM core fragment or TREM fragment, thereby modulating the tRNA pool in the cell.

In an aspect, the disclosure provides a method of contacting a cell, tissue, or subject with a TREM, a TREM core fragment, or a TREM fragment disclosed herein, comprising: contacting the cell, tissue or subject with the TREM, TREM core fragment or TREM fragment, thereby contacting the cell, tissue, or subject with the TREM, TREM core fragment or TREM fragment.

In another aspect, the disclosure provides a method of delivering a TREM, TREM core fragment or TREM fragment to a cell, tissue, or subject, comprising: providing a cell, tissue, or subject, and contacting the cell, tissue, or subject, a TREM, a TREM core fragment, or a TREM fragment disclosed herein.

In an aspect, the disclosure provides a method of modulating a tRNA pool in a cell comprising an endogenous open reading frame (ORF), which ORF comprises a codon having a first sequence, comprising:
optionally, acquiring knowledge of the abundance of one or both of (i) and (ii), e.g., acquiring knowledge of the relative amounts of: (i) and (ii) in the cell, wherein (i) is a tRNA moiety having an anticodon that pairs with the codon of the ORF having a first sequence (the first tRNA moiety) and (ii) is an isoacceptor tRNA moiety having an anticodon that pairs with a codon other than the codon having the first sequence (the second tRNA moiety) in the cell;
contacting the cell with a TREM, a TREM core fragment, or a TREM fragment disclosed herein, wherein the TREM, TREM core fragment or TREM fragment has an anticodon that pairs with: the codon having the first sequence; or the codon other than the codon having the first sequence, in an amount and/or for a time sufficient to modulate the relative amounts of the first IRNA moiety and the second tRNA moiety in the cell,
thereby modulating the tRNA pool in the cell.

In another aspect, the disclosure provides a method of modulating a tRNA pool in a subject having an ORF, which ORF comprises a codon having a first sequence, comprising: optionally, acquiring knowledge of the abundance of one or both of (i) and (ii), e.g., acquiring knowledge of the relative amounts of: (i) and (ii) in the subject, wherein (i) is a tRNA moiety having an anticodon that pairs with the codon of the ORF having a first sequence (the first tRNA moiety) and (ii) is an isoacceptor tRNA moiety having an anticodon that pairs with a codon other than the codon having the first sequence (the second tRNA moiety) in the subject;
contacting the subject with a TREM, a TREM core fragment, or a TREM fragment disclosed herein, wherein the TREM, TREM core fragment or TREM fragment has an anticodon that pairs with: the codon having the first sequence; or the codon other than the codon having the first sequence, in an amount and/or for a time sufficient to modulate the relative amounts of the first tRNA moiety and the second tRNA moiety in the subject.
thereby modulating the tRNA pool in the subject.

In an aspect, the disclosure provides a method of modulating a tRNA pool in a subject having an endogenous ORF comprising a codon comprising a synonymous mutation (a synonymous mutation codon or SMC), comprising:
providing a composition comprising a TREM, a TREM core fragment, or a TREM fragment disclosed herein, wherein the TREM, TREM core fragment or TREM fragment comprises an isoacceptor tRNA moiety comprising an anticodon sequence that pairs with the SMC (the TREM);
contacting the subject with the composition in an amount and/or for a time sufficient to modulate the tRNA pool in the subject,
thereby modulating the tRNA pool in the subject.

In another aspect, the disclosure provides a method of modulating a tRNA pool in a cell comprising an endogenous ORF comprising a codon comprising a SMC, comprising:
providing a composition comprising a TREM, a TREM core fragment, or a TREM fragment disclosed herein, wherein the TREM, TREM core fragment or TREM fragment comprises an isoacceptor tRNA moiety comprising an anticodon sequence that pairs with the SMC (the TREM);
contacting the cell with the composition comprising a TREM in an amount and/or for a time sufficient to modulate the tRNA pool in the cell,
thereby modulating the tRNA pool in the cell.

In an aspect, the disclosure provides a method of modulating expression of a protein in a cell, wherein the protein is encoded by a nucleic acid comprising an ORF, which ORF comprises a codon having a mutation, comprising:
contacting the cell with a composition comprising a TREM, a TREM core fragment, or a TREM fragment disclosed herein in an amount and/or for a time sufficient to modulate expression of the encoded protein,
wherein the TREM, TREM core fragment or TREM fragment has an anticodon that pairs with the codon having the mutation,
thereby modulating expression of the protein in the cell.

In another aspect, the disclosure provides a method of modulating expression of a protein in a subject, wherein the protein is encoded by a nucleic acid comprising an endogenous ORF, which ORF comprises a codon having a mutation, comprising:
contacting the subject with a composition comprising a TREM, a TREM core fragment, or a TREM fragment disclosed herein, in an amount and/or for a time sufficient to modulate expression of the encoded protein,
wherein the TREM, TREM core fragment or TREM fragment has an anticodon that pairs with the codon having the mutation,
thereby modulating expression of the protein in the subject.

In an embodiment of any of the methods disclosed herein, the mutation in the ORF is a nonsense mutation, e.g., resulting in a premature stop codon chosen from UAA, UGA or UAG. In an embodiment, the stop codon is UAA. In an embodiment, the stop codon is UGA. In an embodiment, the stop codon is UAG.

In an embodiment of any of the methods disclosed herein, the TREM comprises an anticodon that pairs with a stop codon.

TREMs of the disclosure include TREMs, TREM core fragments and TREM fragments. TREMs, TREM core fragments or TREM fragments can be modified with non-naturally occurring modifications to, e.g., increase the level and/or activity (e.g., stability) of the TREM. Pharmaceutical TREM compositions, e.g., comprising TREMs having a non-naturally occurring modification, can be administered to cells, tissues or subjects to modulate these functions, e.g., in vitro or in vivo. Disclosed herein are TREMs, TREM core fragments or TREM fragments comprising non-naturally occurring modifications, TREM compositions, preparations, methods of making TREM compositions and preparations, and methods of using the same.

In an embodiment, the TREM, TREM core fragment, and TREM fragments comprise a non-naturally occurring modification that improves stability or enhances activity of the TREM, TREM core fragment, or TREM fragment.

Additional features of any of the aforesaid TREMs, TREM core fragments, TREM fragments, TREM compositions, preparations, methods of making TREM compositions and preparations, and methods of using TREM compositions and preparations include one or more of the features in the Enumerated Embodiments, Figures, Description, Examples, or Claims.

Those skilled in the art will recognize or be able to ascertain using no more than routine experimentation, many equivalents to the specific embodiments of the invention described herein. Such equivalents are intended to be encompassed by the following Enumerated Embodiments, Drawings, Description, Examples, or Claims.

### BRIEF DESCRIPTION OF DRAWINGS

FIG. 1 is a depiction of a representative TREM with a summary of a design guidances (e.g., Design Guidance 1-6) described herein.

FIG. 2 is a table summarizing exemplary TREMs, TREM core fragments, and TREM fragments described herein. The sequence of each TREM. TREM core fragment, and TREM fragment is provided, and the chemical modification profile is annotated as follows: : r: ribonucleotide; m: 2′-OMe; *: PS linkage; f: 2′-fluoro; moe: 2′-moe; d: deoxyribonucleotide; 5MeC: 5-methylcytosine. Thus, for example, mA represents 2′-O-methyl adenosine, moe5MeC represents 2′-MOE nucleotide with 5-methylcytosine nucleobase, and dA represents an adenosine deoxyribonucleotide. The table also provides mass spectrometric characterization of each TREM, TREM core fragment, and TREM fragment, along with results from the activity screens described in Examples 6 and 7. The results from the activity screens are in the columns titled “A” (described in Example 6), “B,” “C,” and “D” (all three described in Example 7).

FIG. 3 is an image showing PTC readthrough activity of exemplary TREMs described herein in four cell lines as outlined in both Examples 6 and 7. Activity is shown as log 2 fold change over a control unmodified TREM.

FIG. 4 is an image showing Western blot analysis of full-length GLA protein rescue in Fabry patient fibroblasts and normal healthy fibroblasts upon administration of exemplary TREMs described herein, as described in Example 11.

FIGS. 5A-5E depict the results of time course and dose-response studies in Fabry patient fibroblasts and normal healthy fibroblasts upon administration of exemplary TREMs described herein, as described in Example 11.

FIG. 6 is a graph illustrating the rescue of GLA activity in Fabry patient fibroblasts upon administration of exemplary TREMs described herein, as outlined in Example 11.

FIG. 7 is a set of graphs illustrating in vivo PTC readthrough and target engagement of a TREM. FIG. 7A is a graph depicting dose-dependent expression of luciferase in the liver from a plasmid following hydrodynamic delivery. FIG. 7B is a graph illustrating rescue of a luciferase gene with a PTC mutation with a plasmid expressing the corresponding TREM.

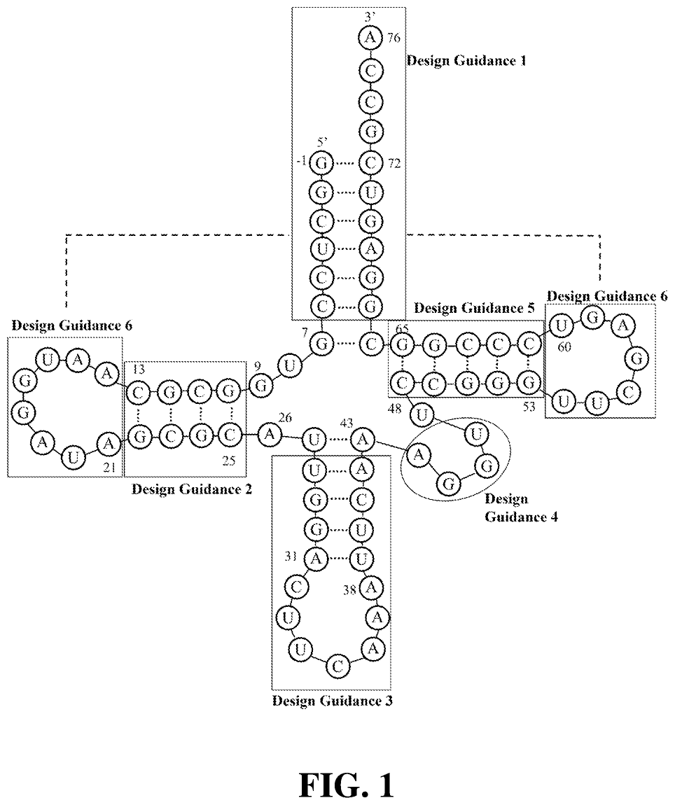

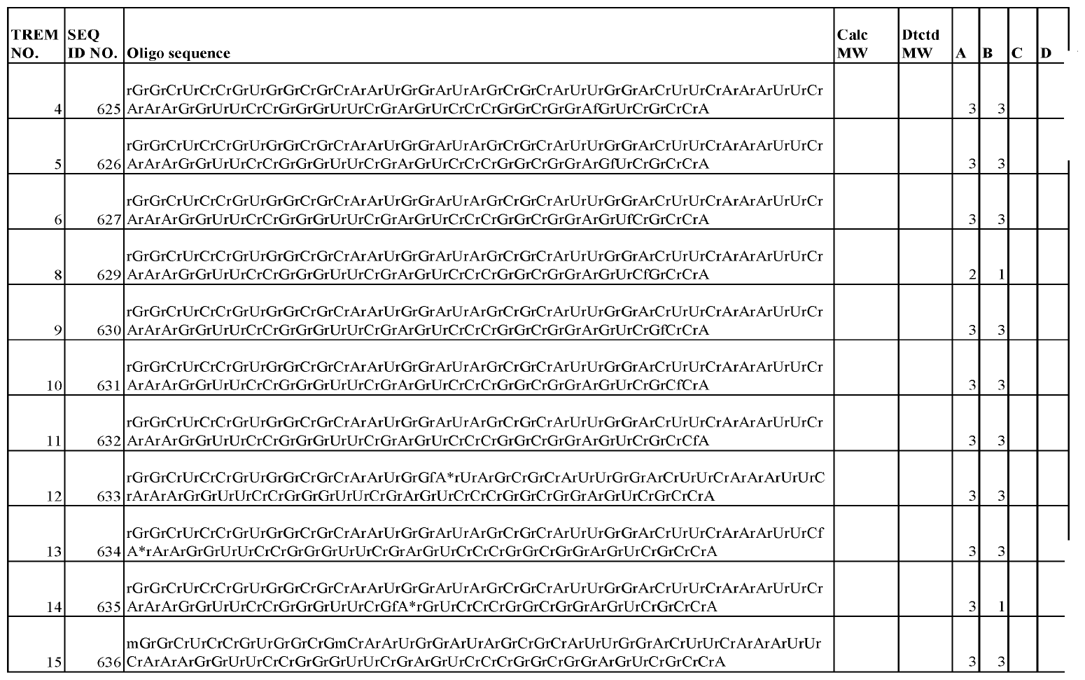

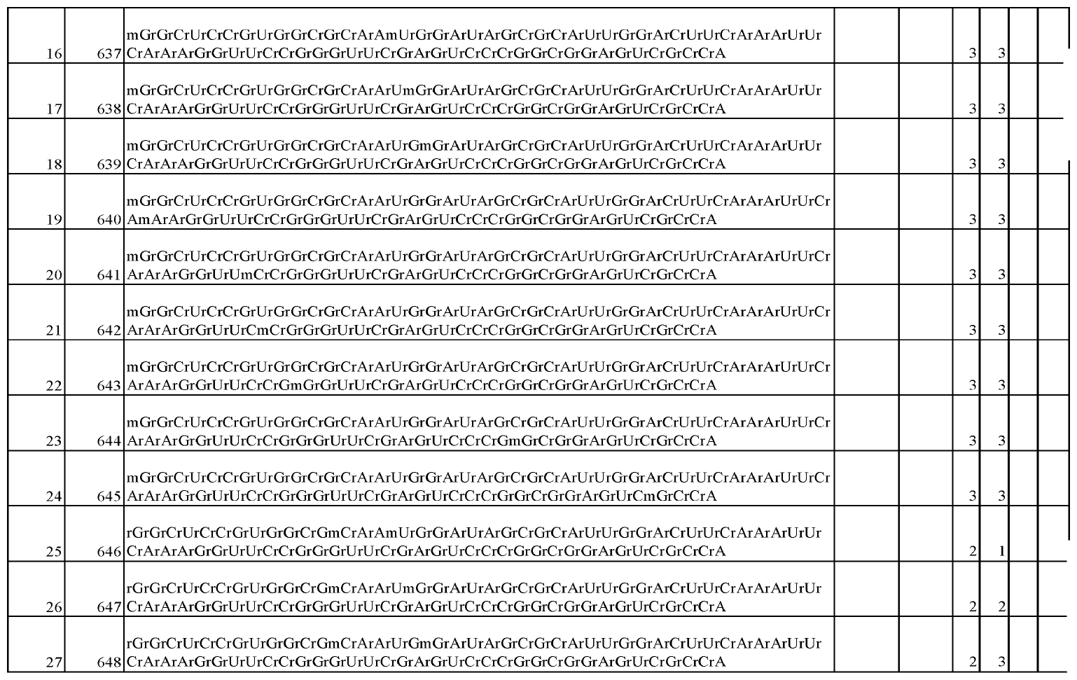

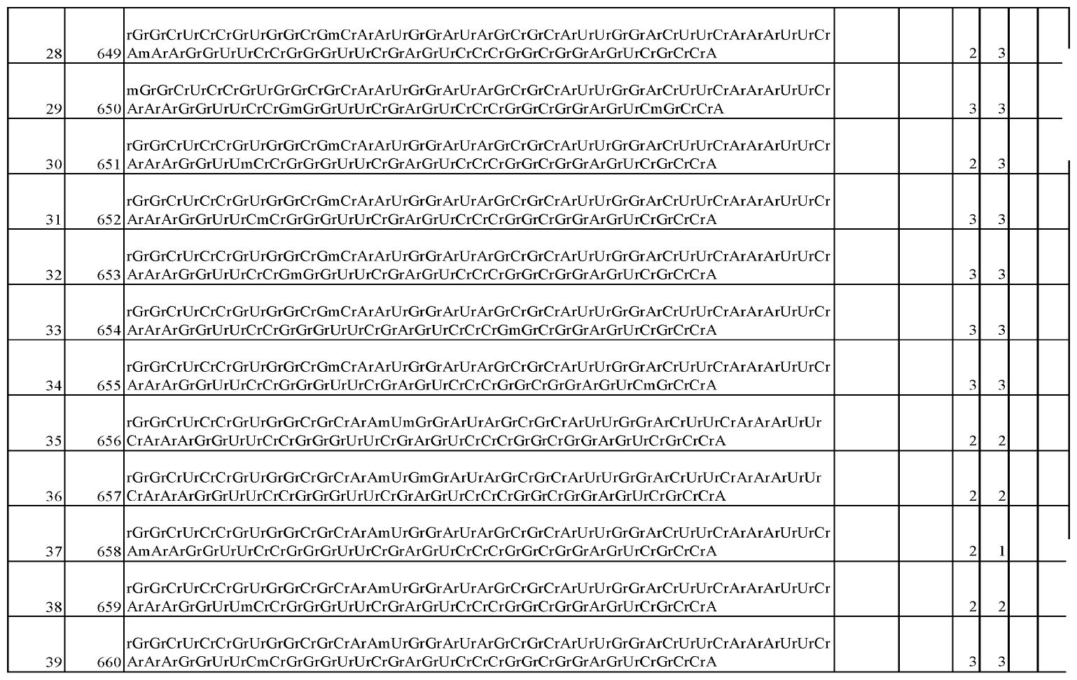

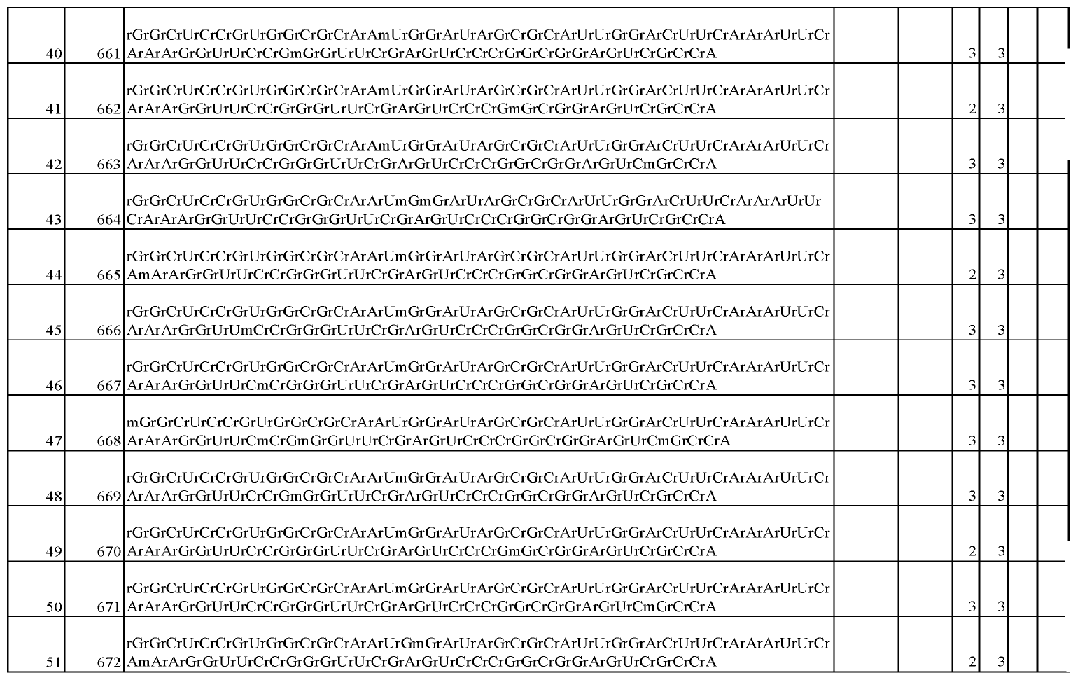

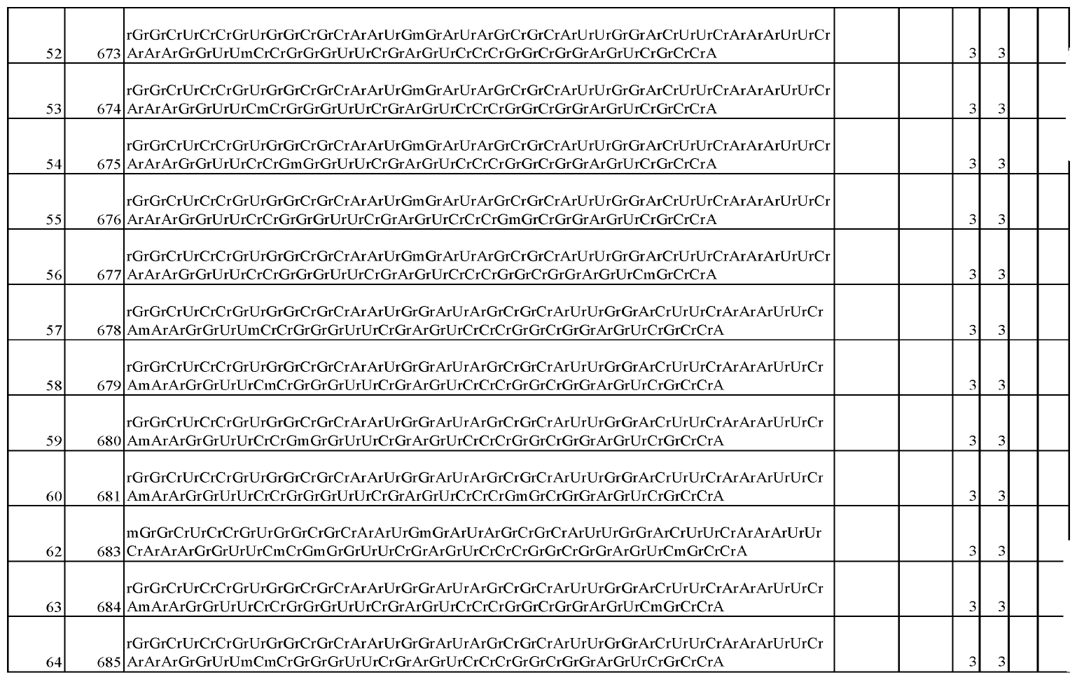

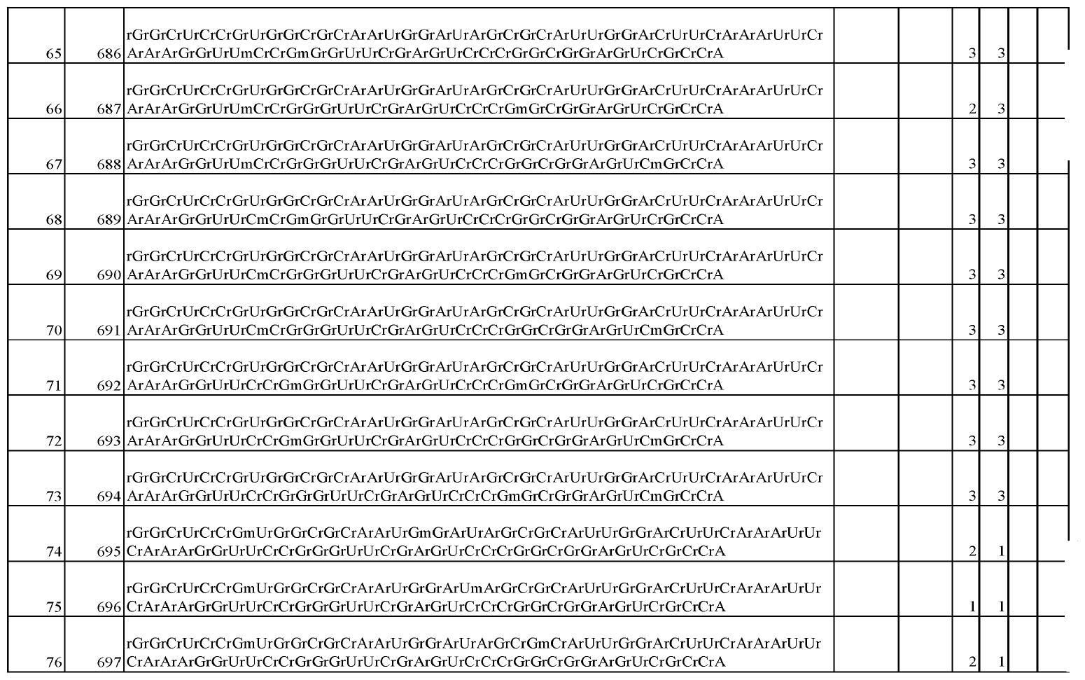

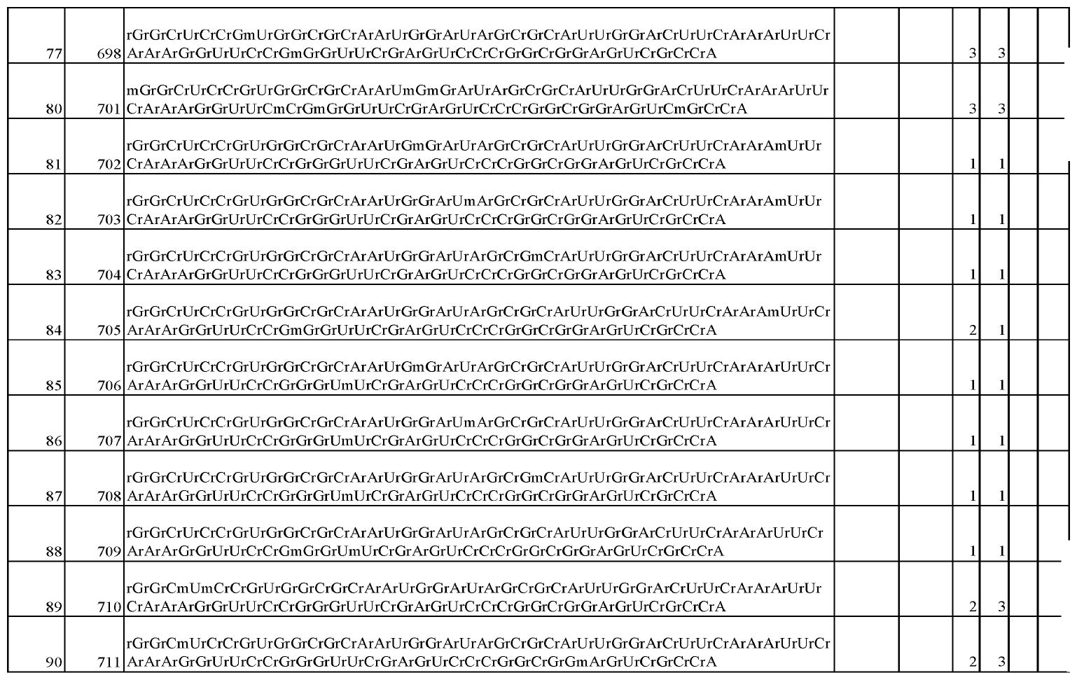

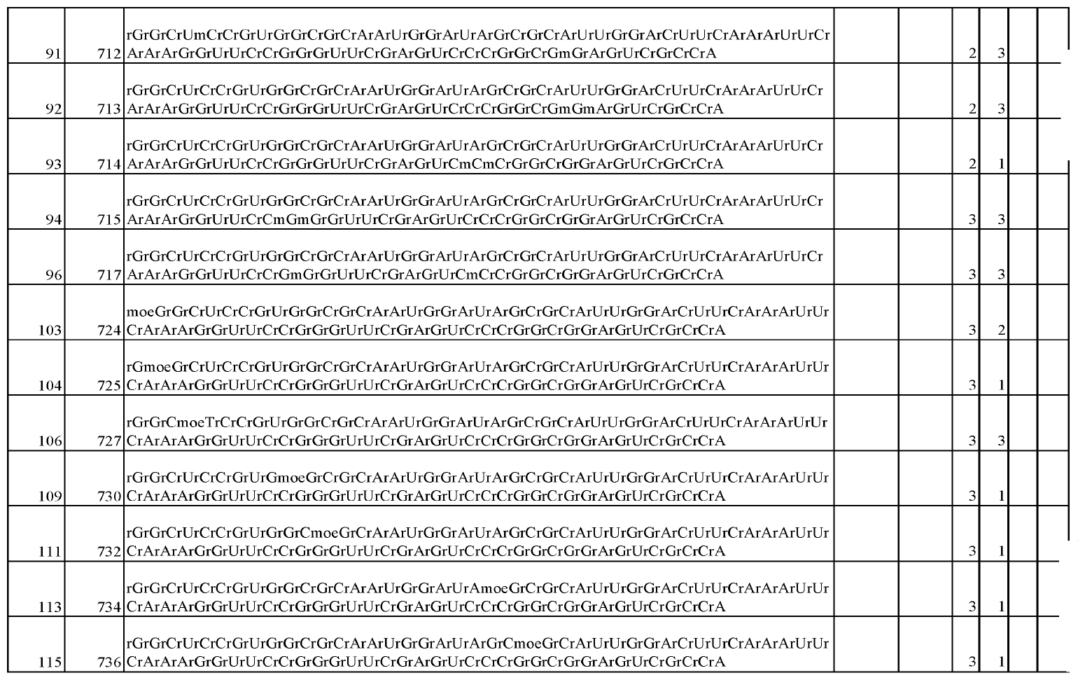

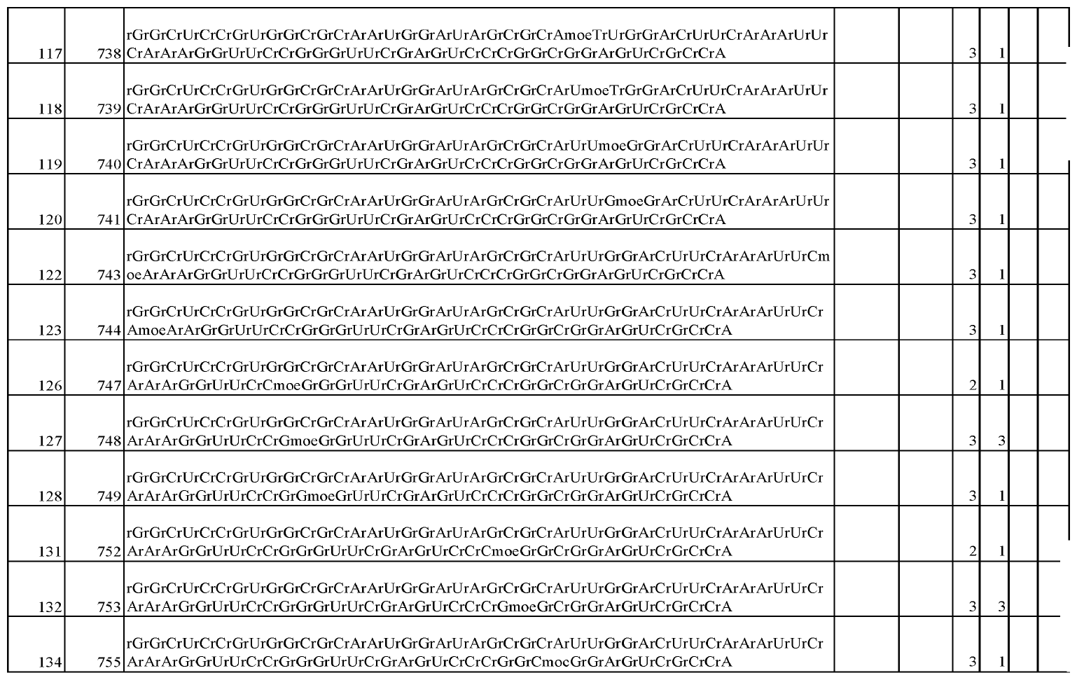

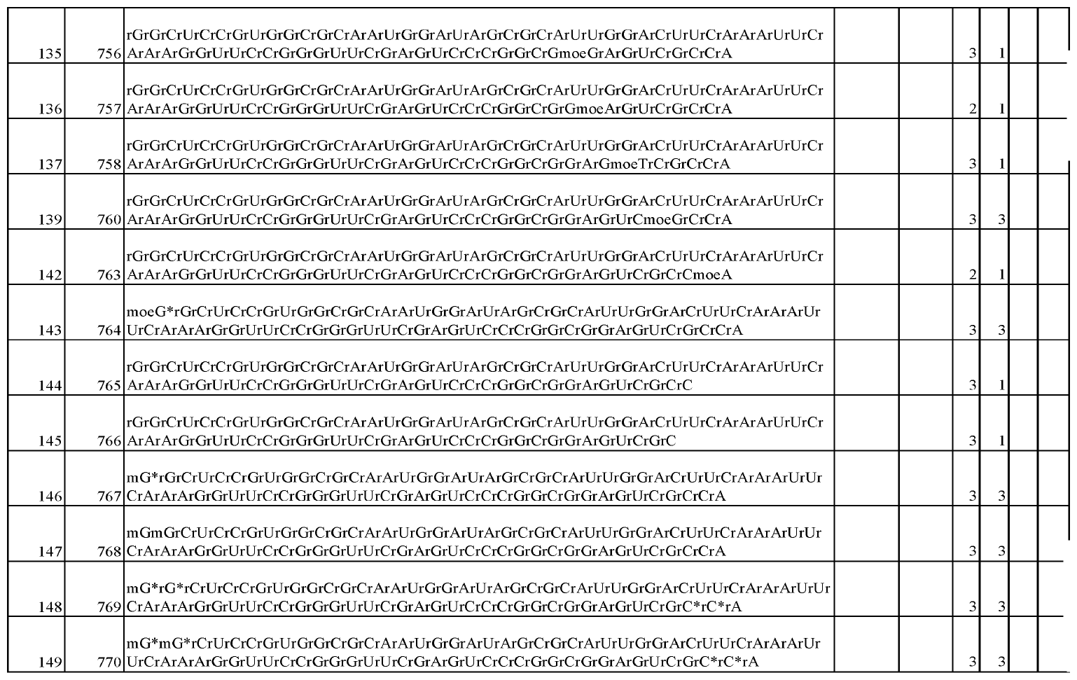

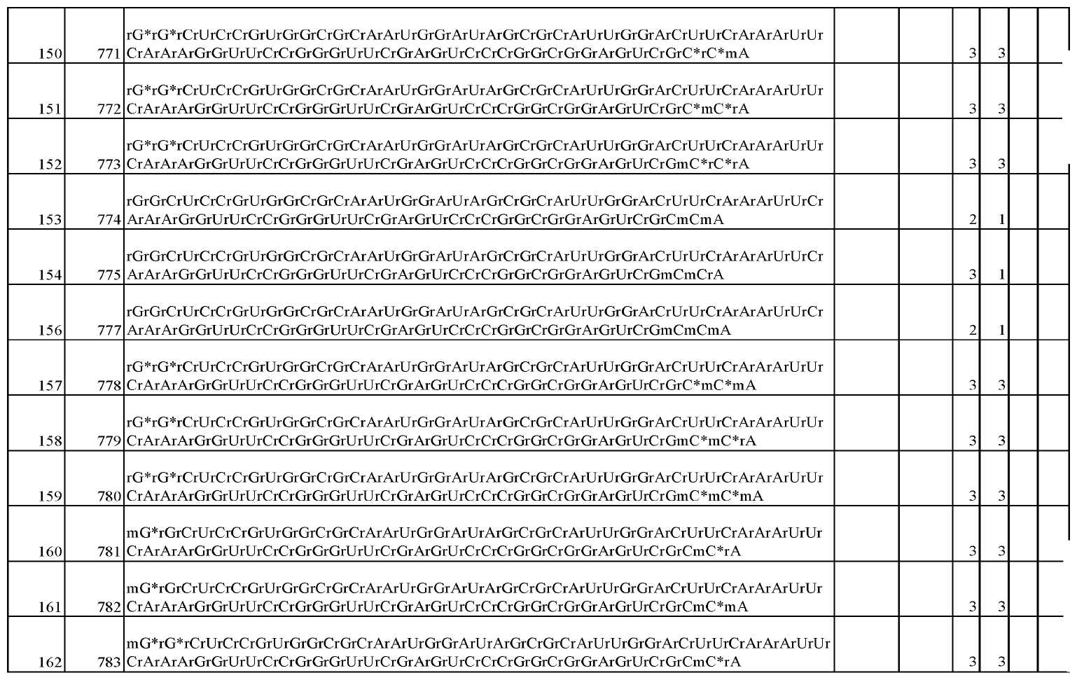

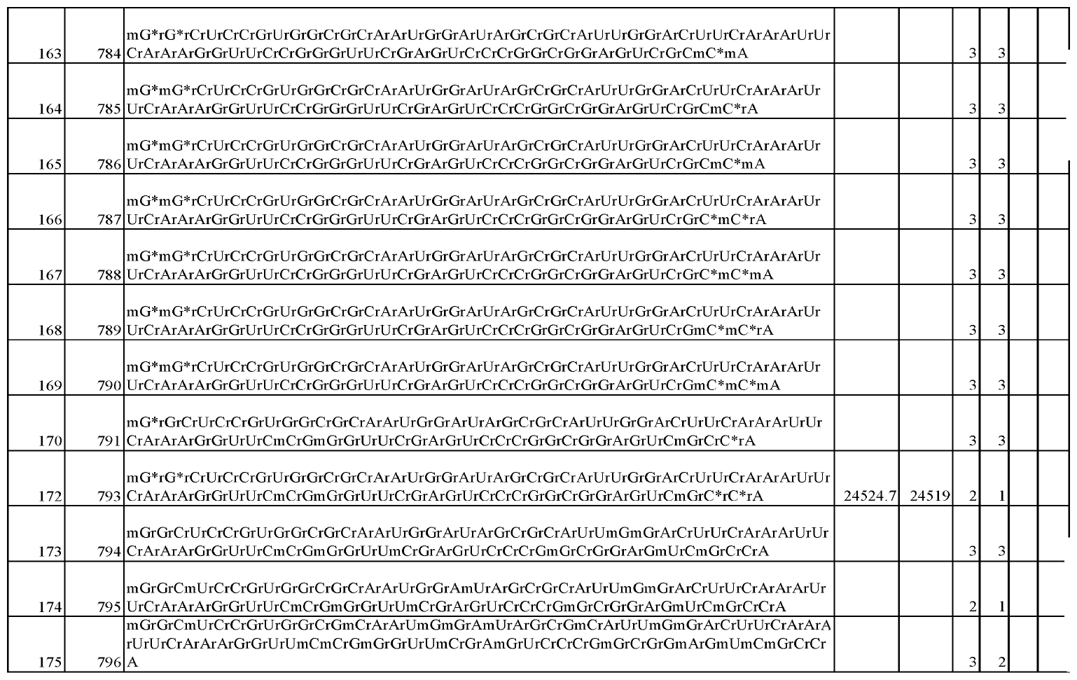

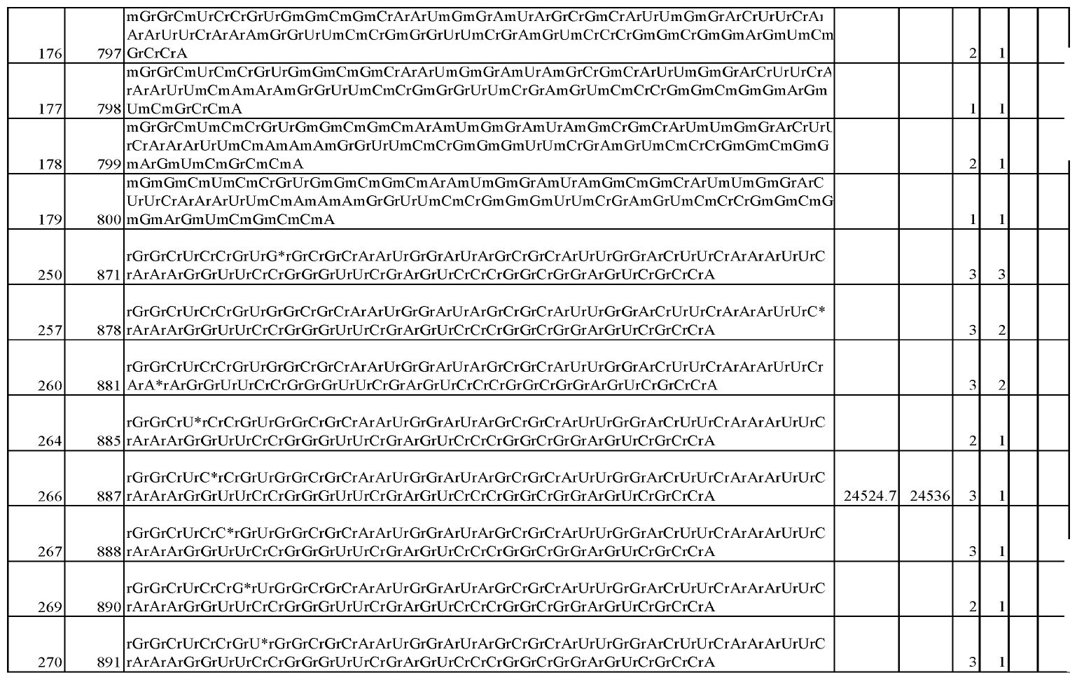

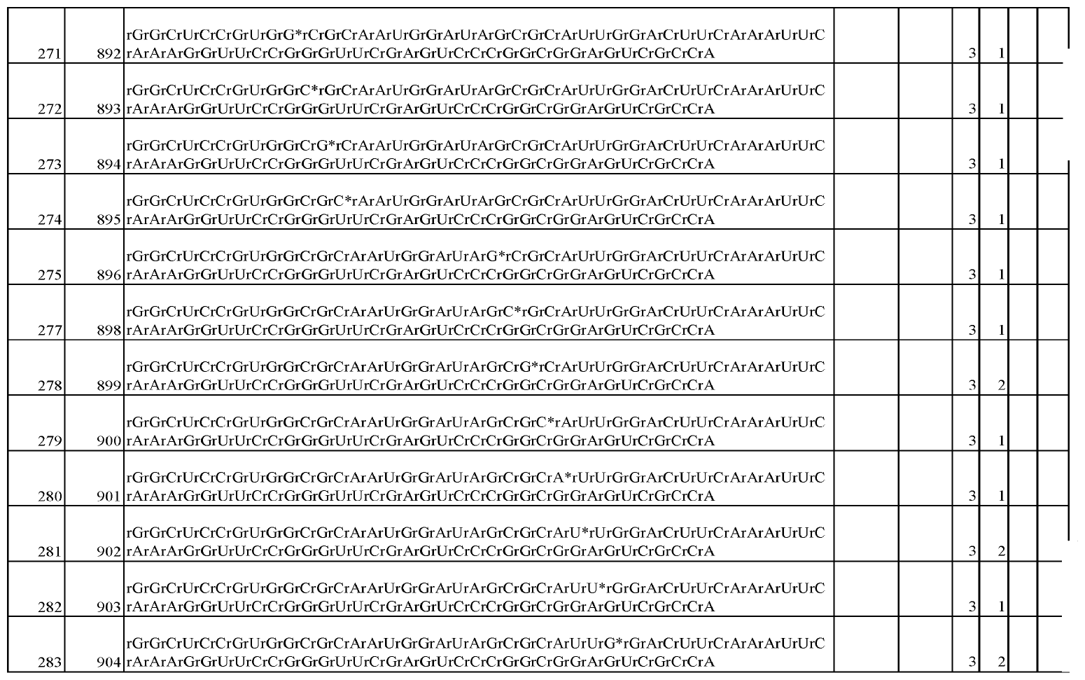

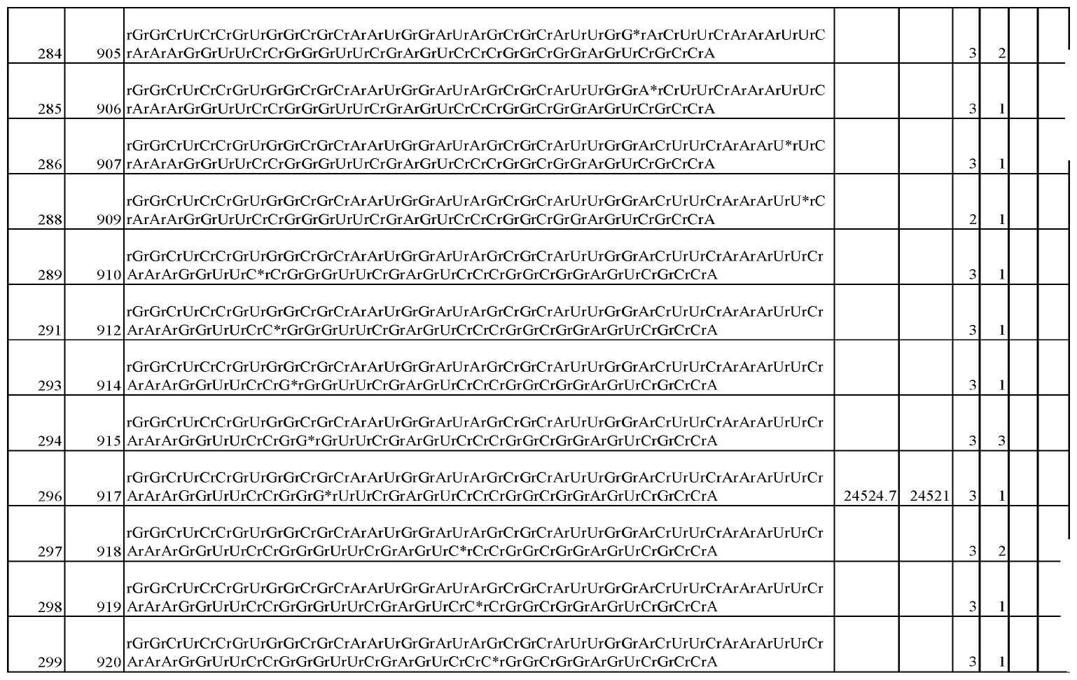

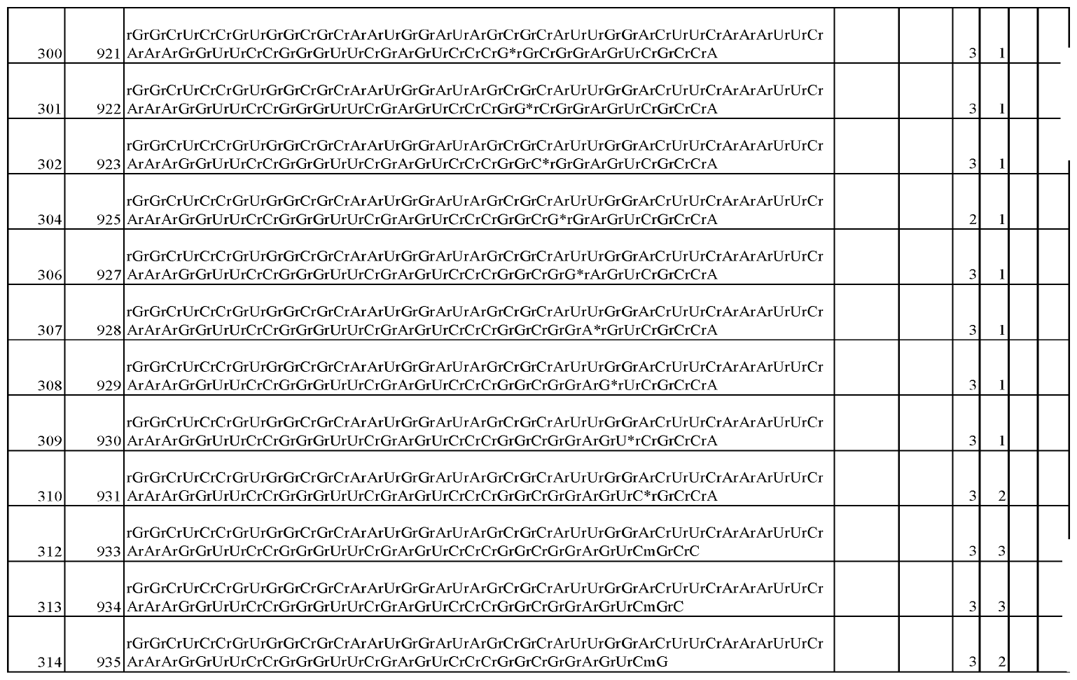

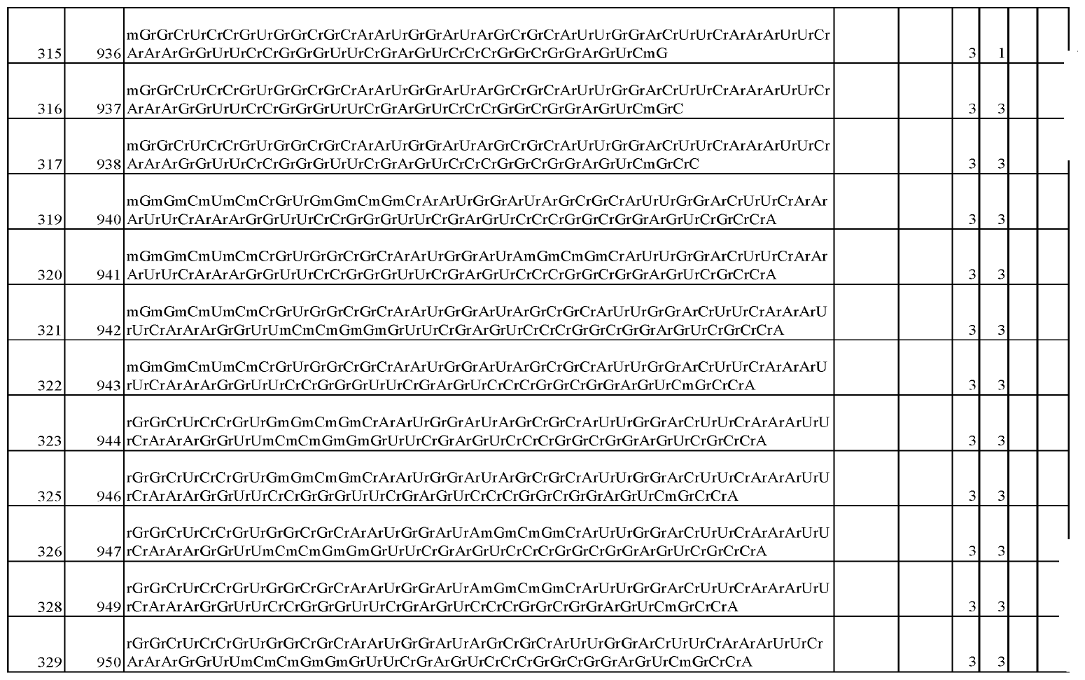

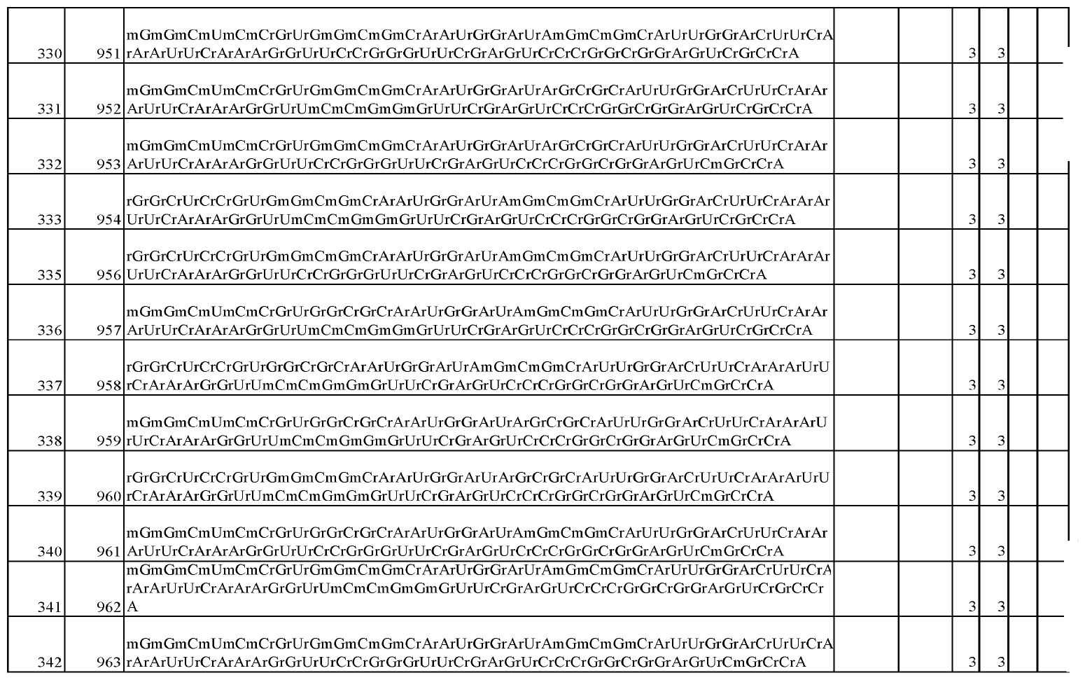

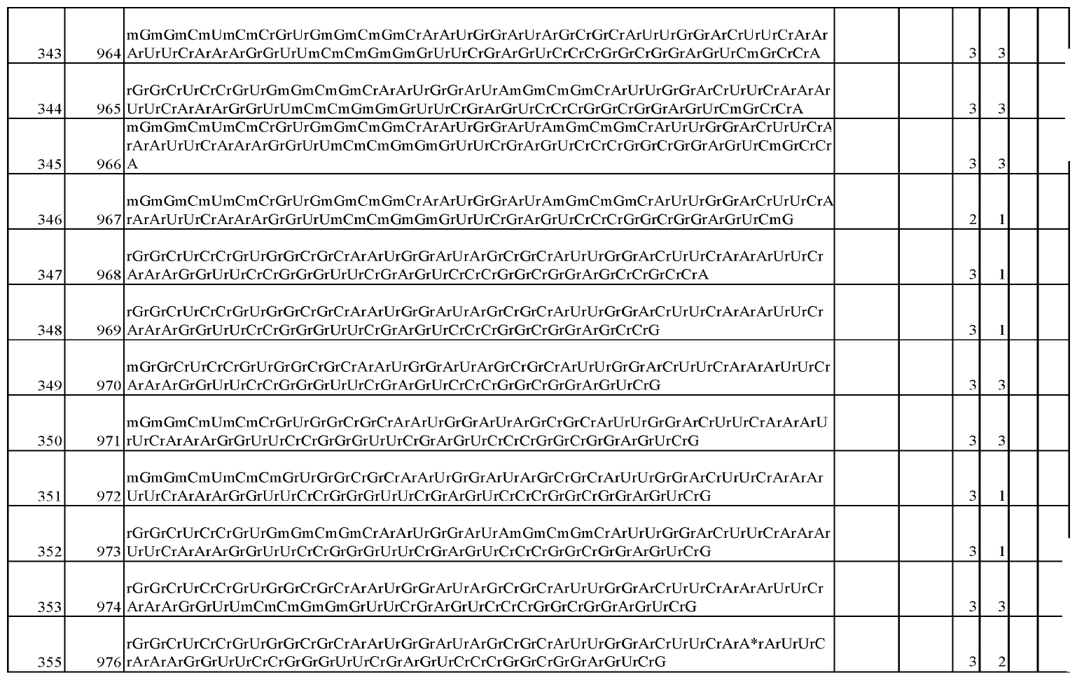

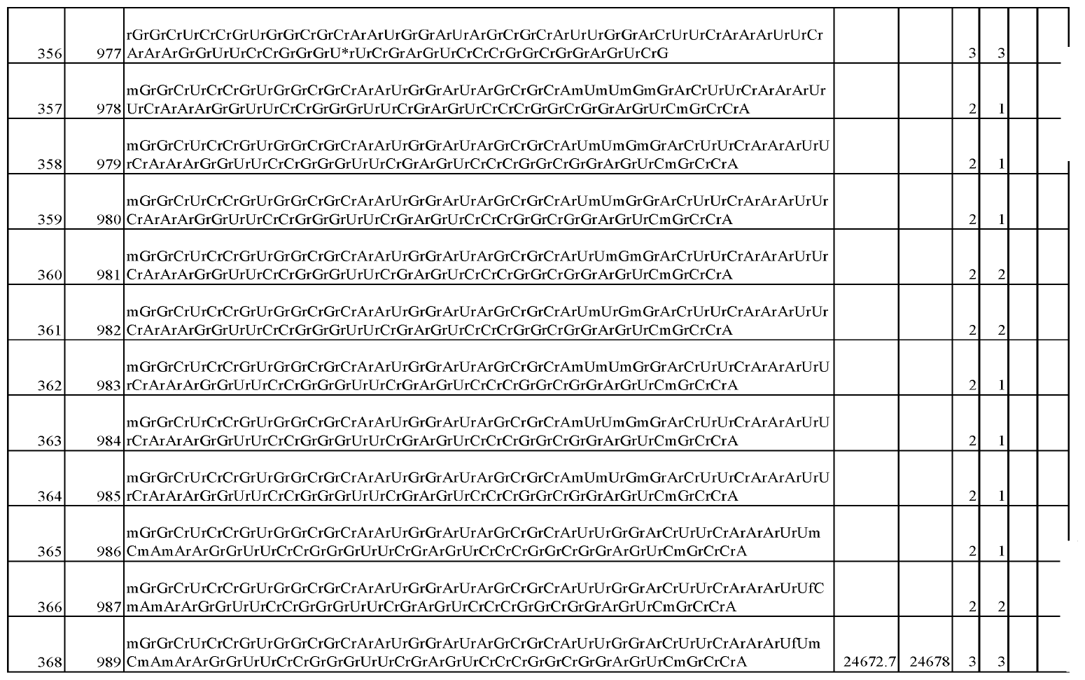

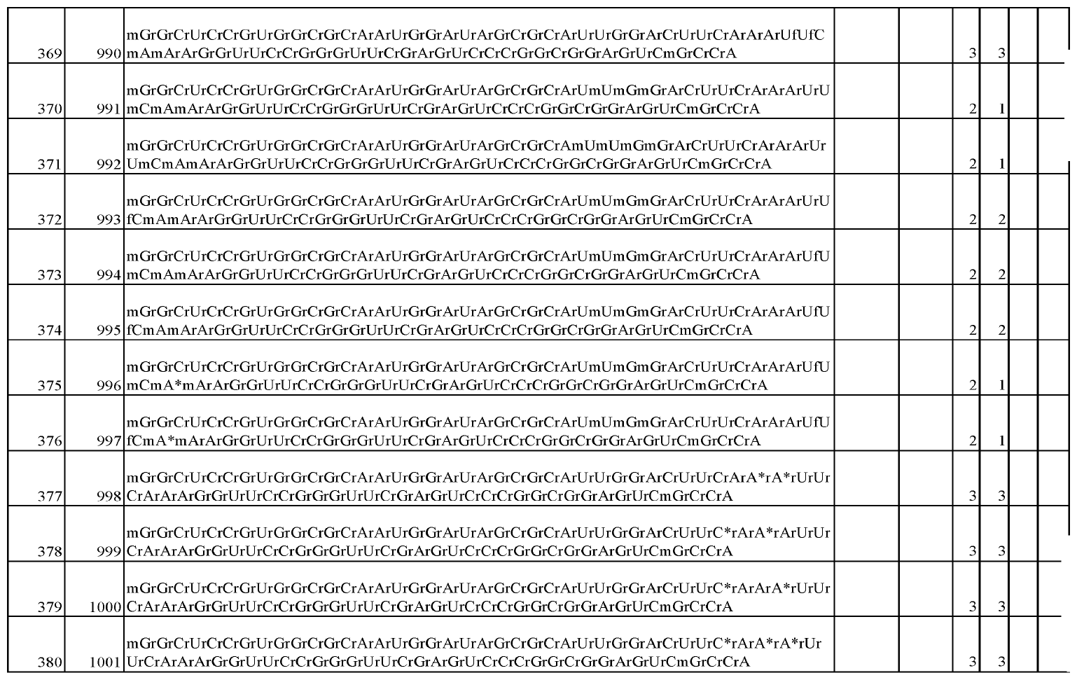

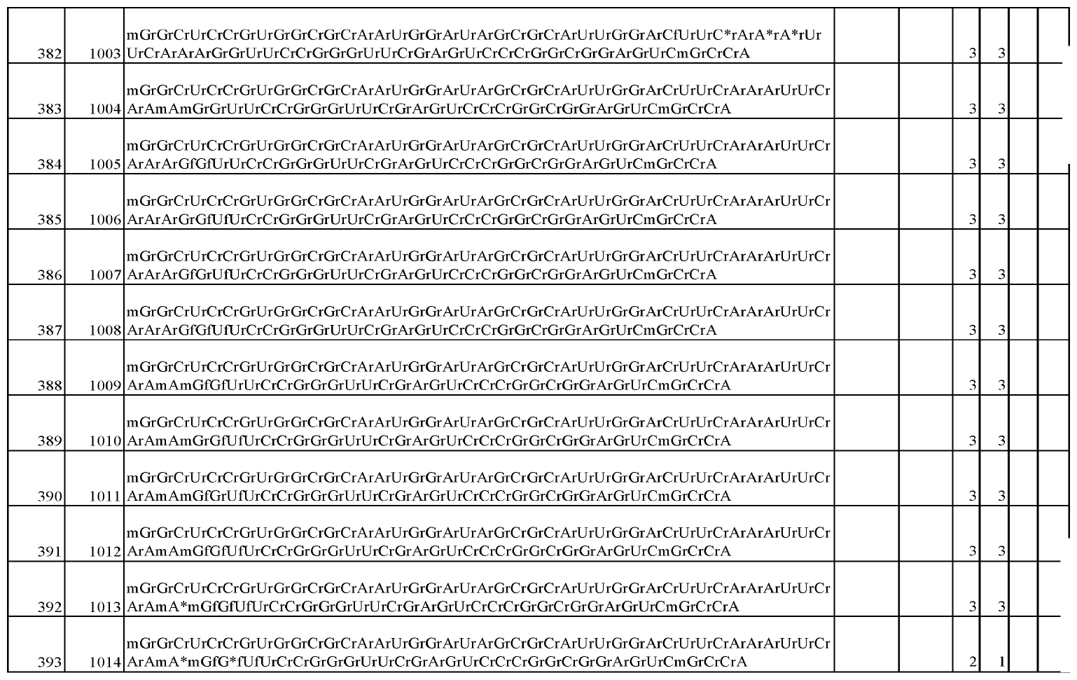

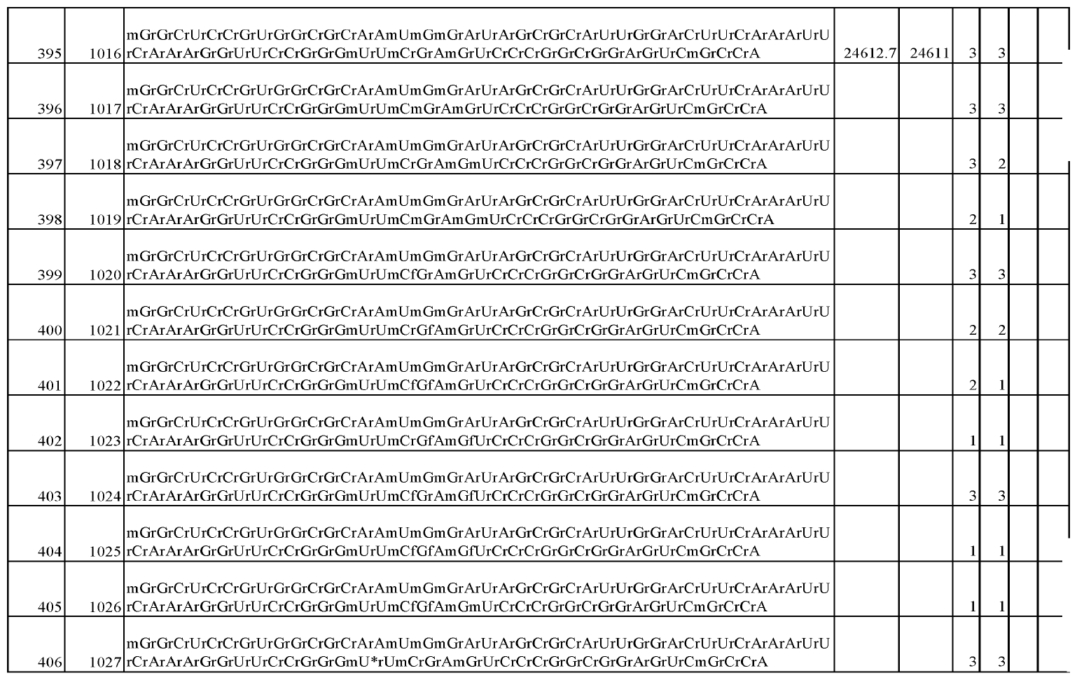

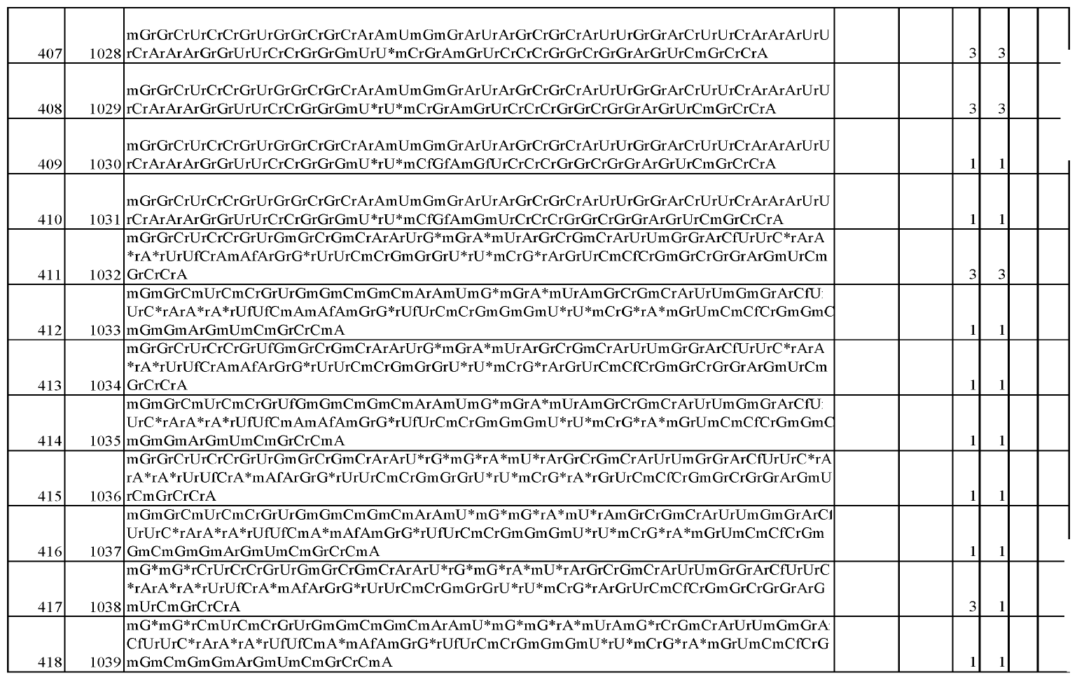

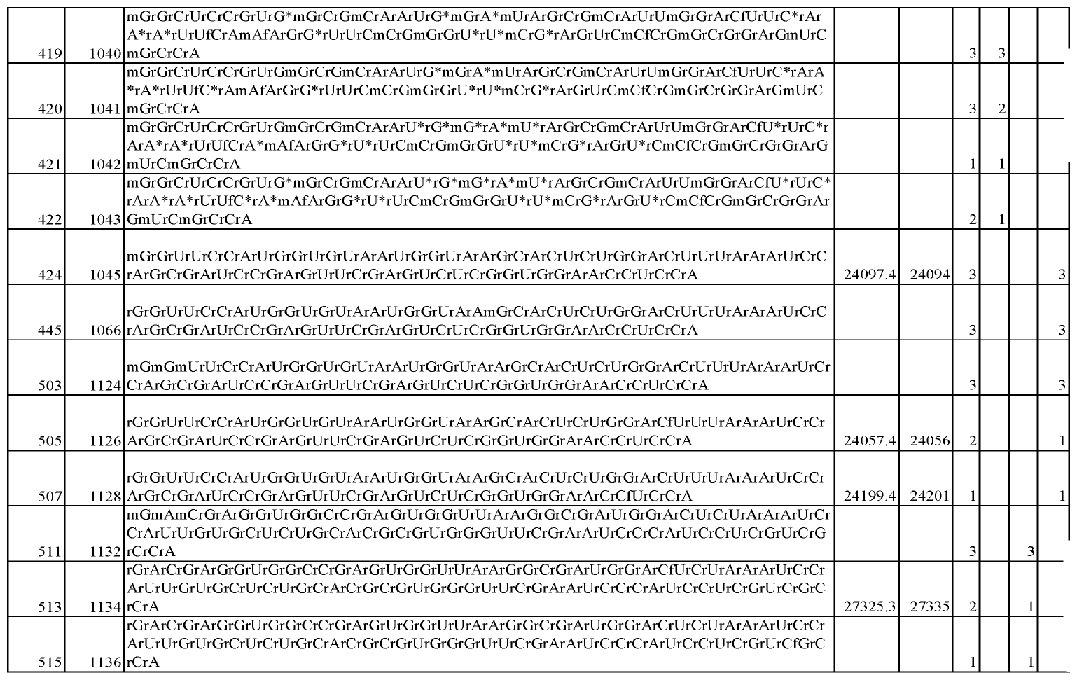

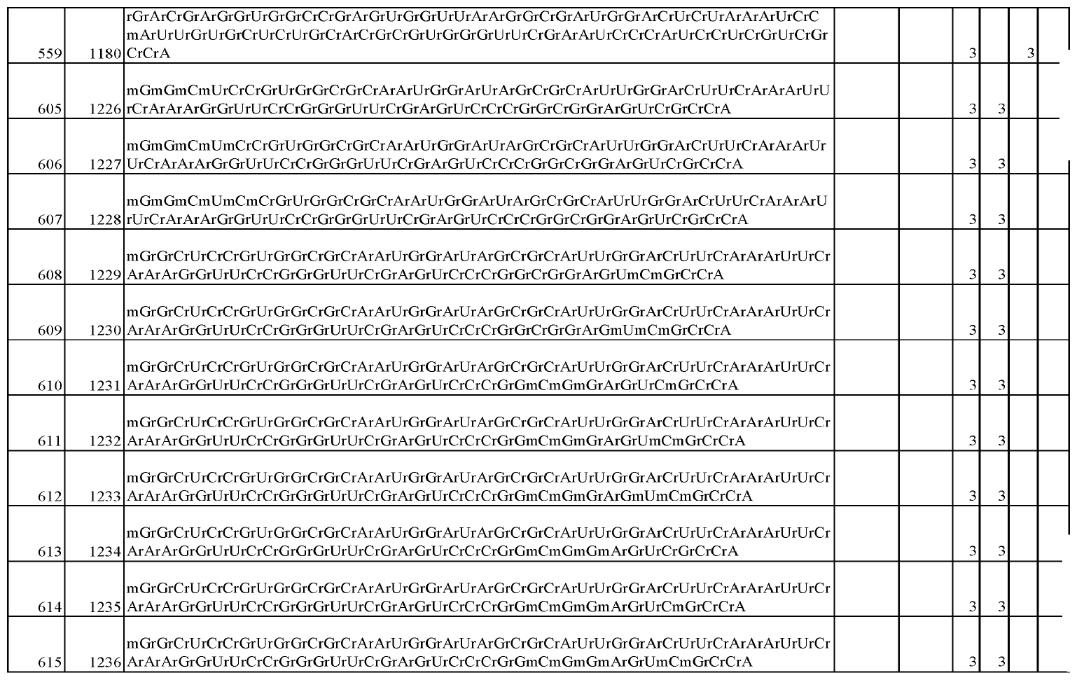

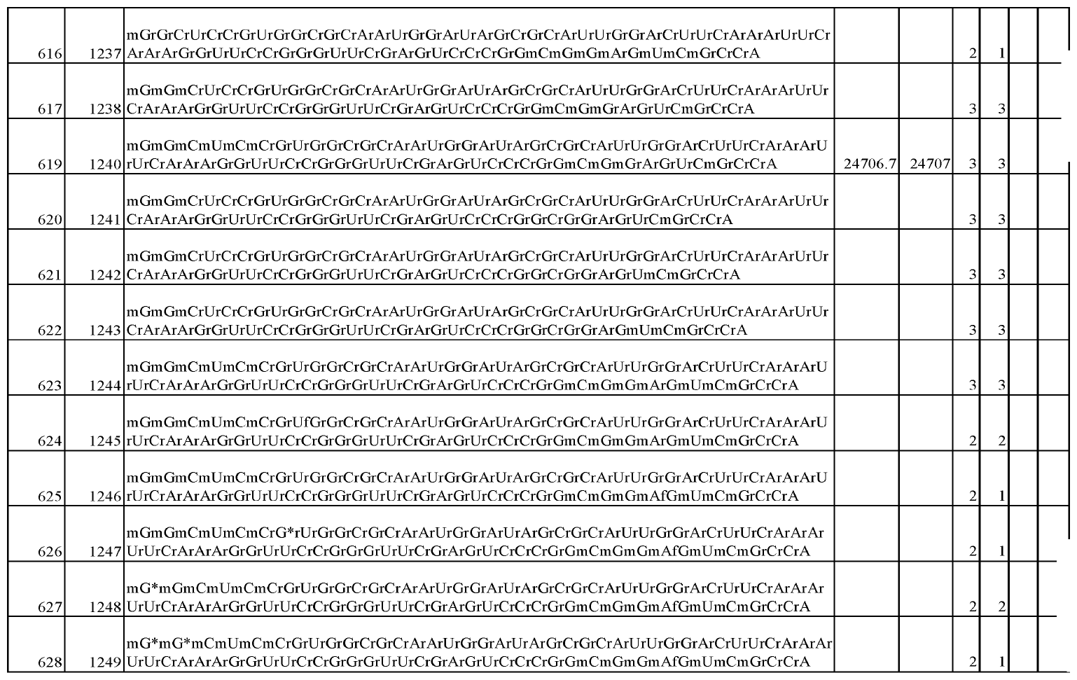

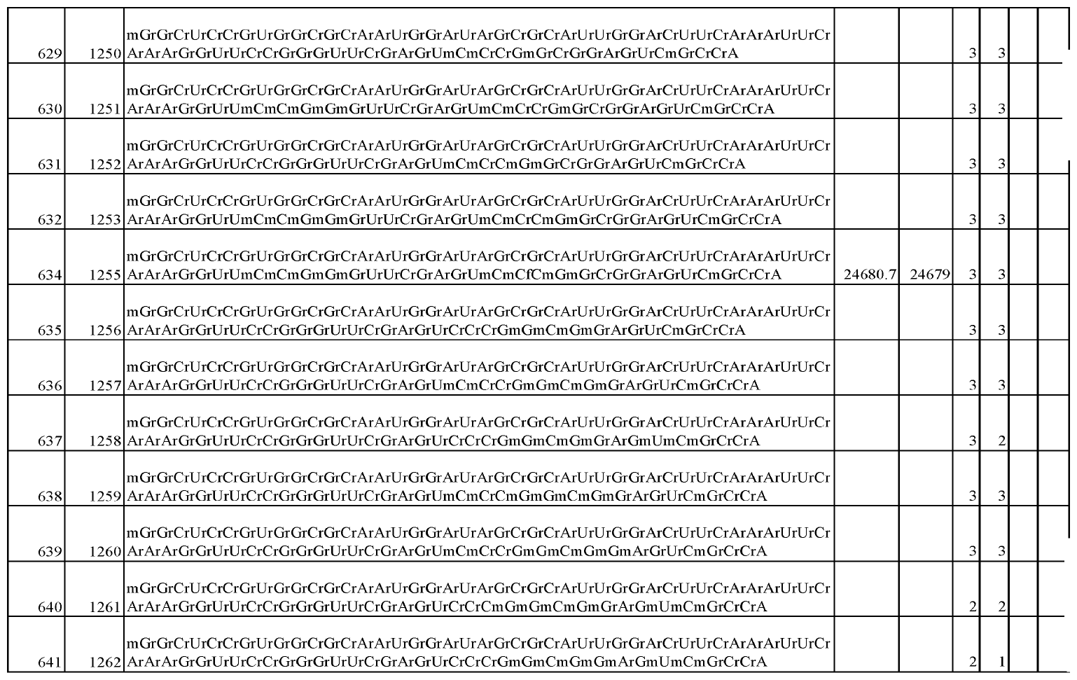

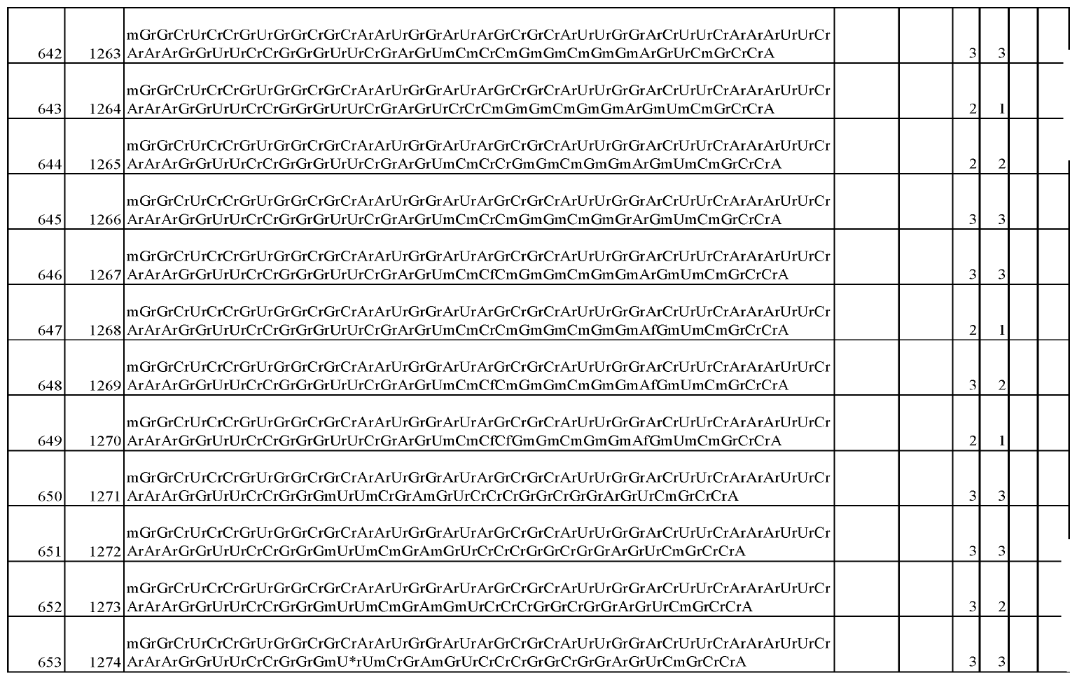

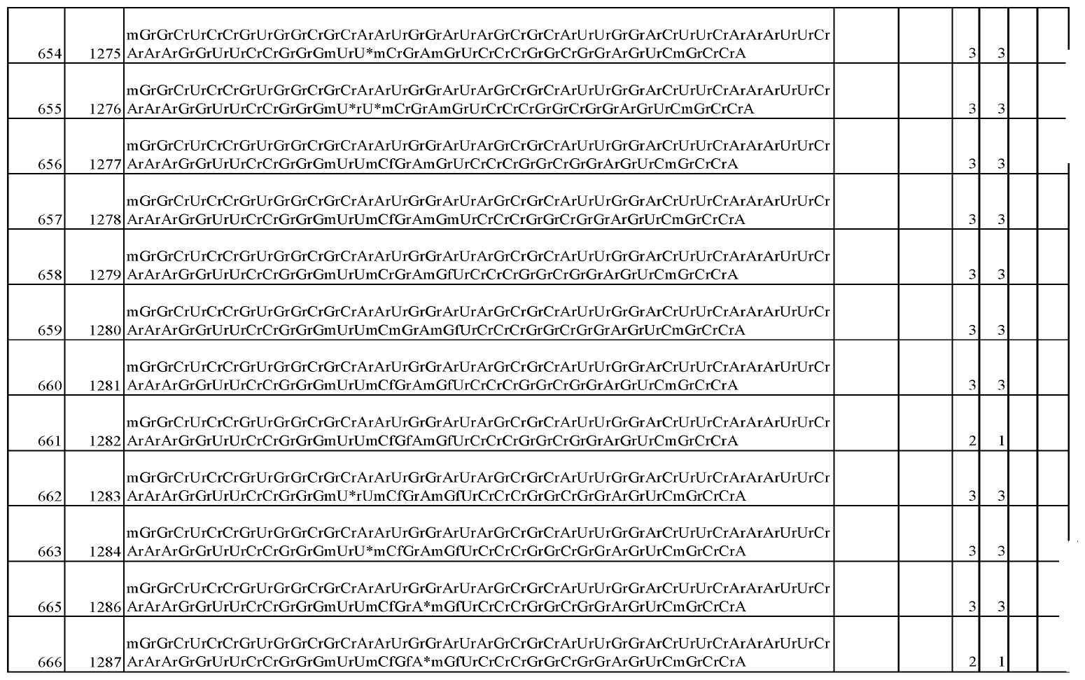

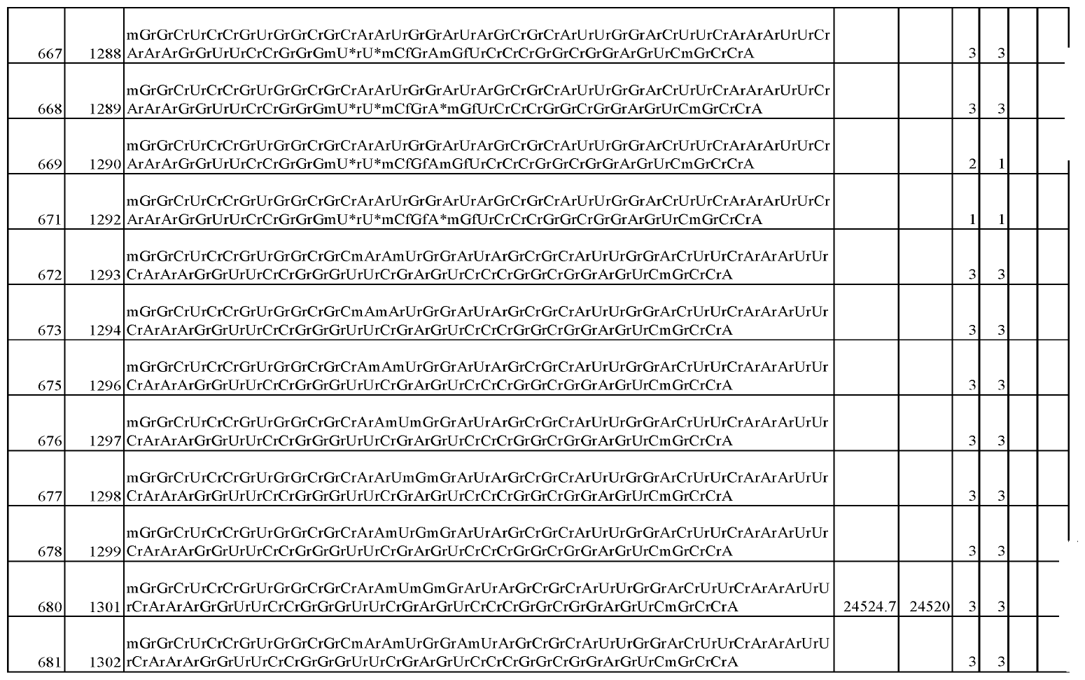

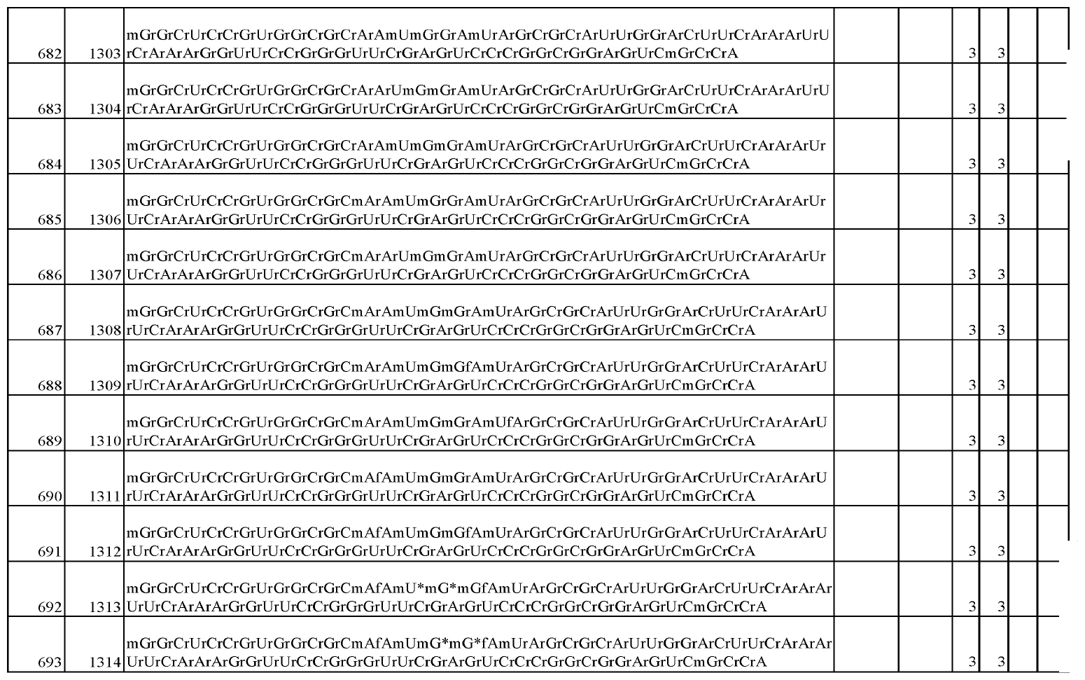

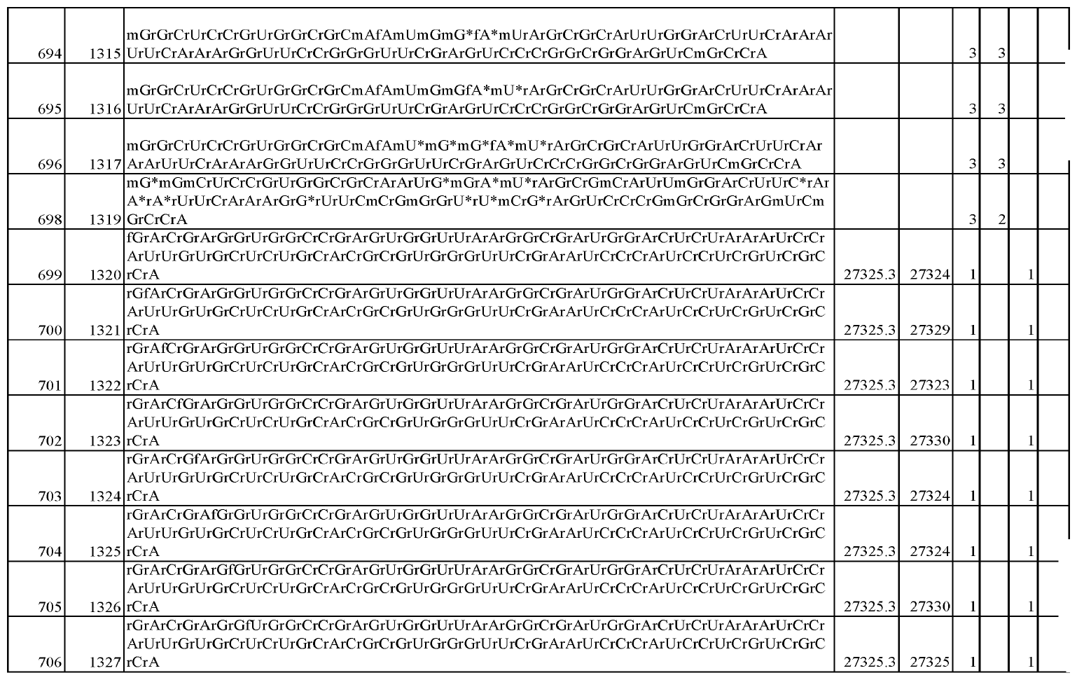

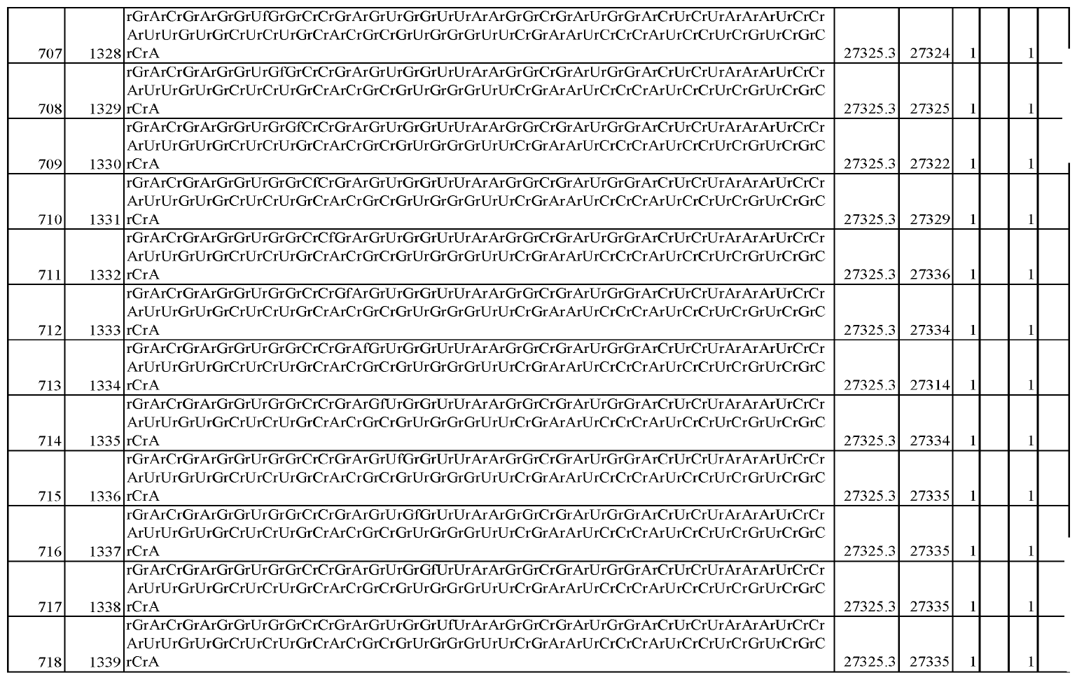

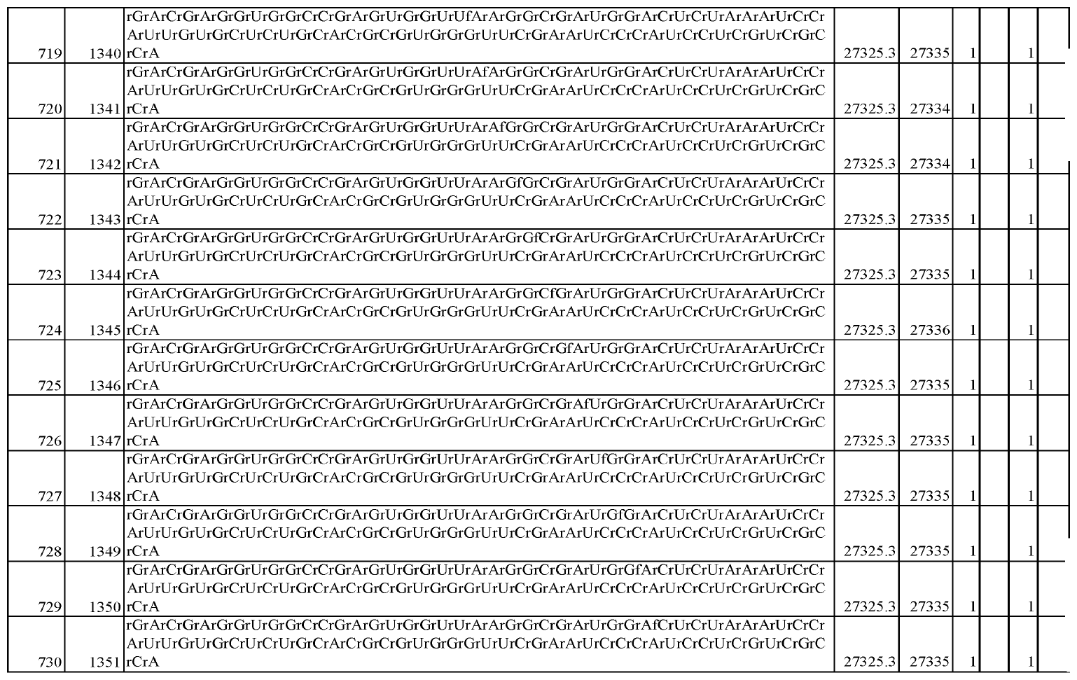

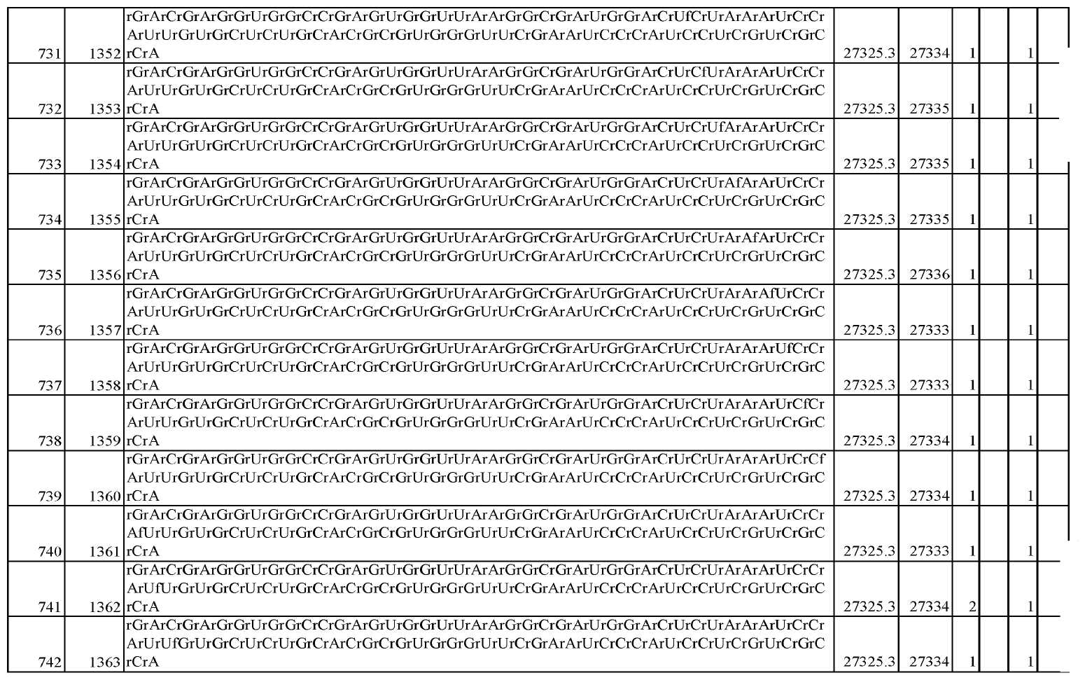

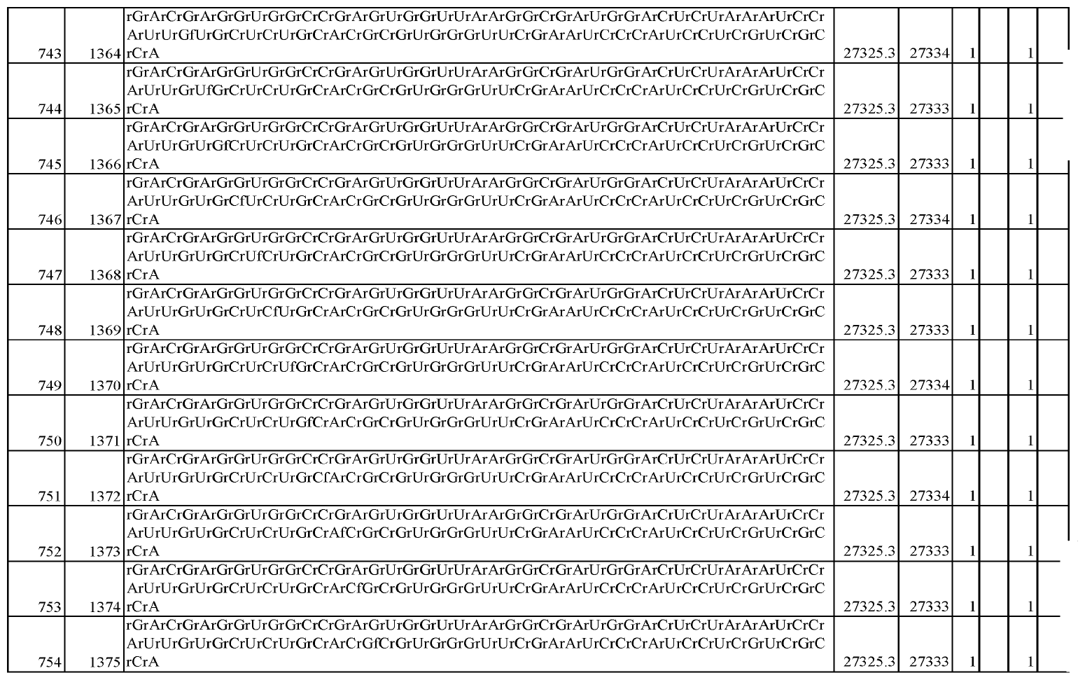

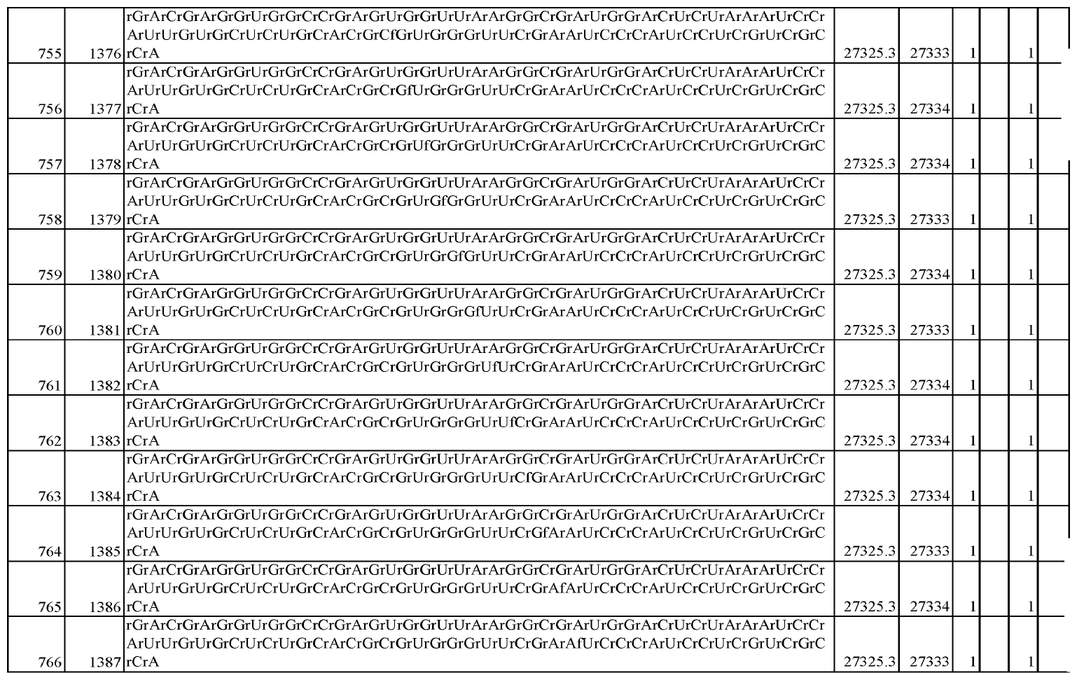

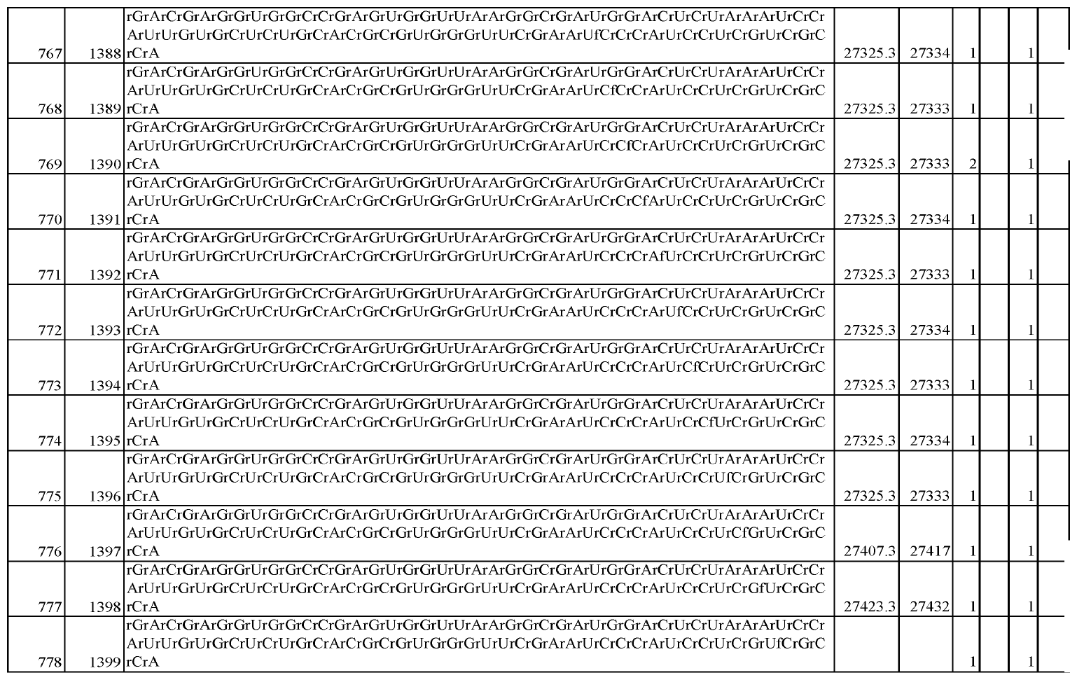

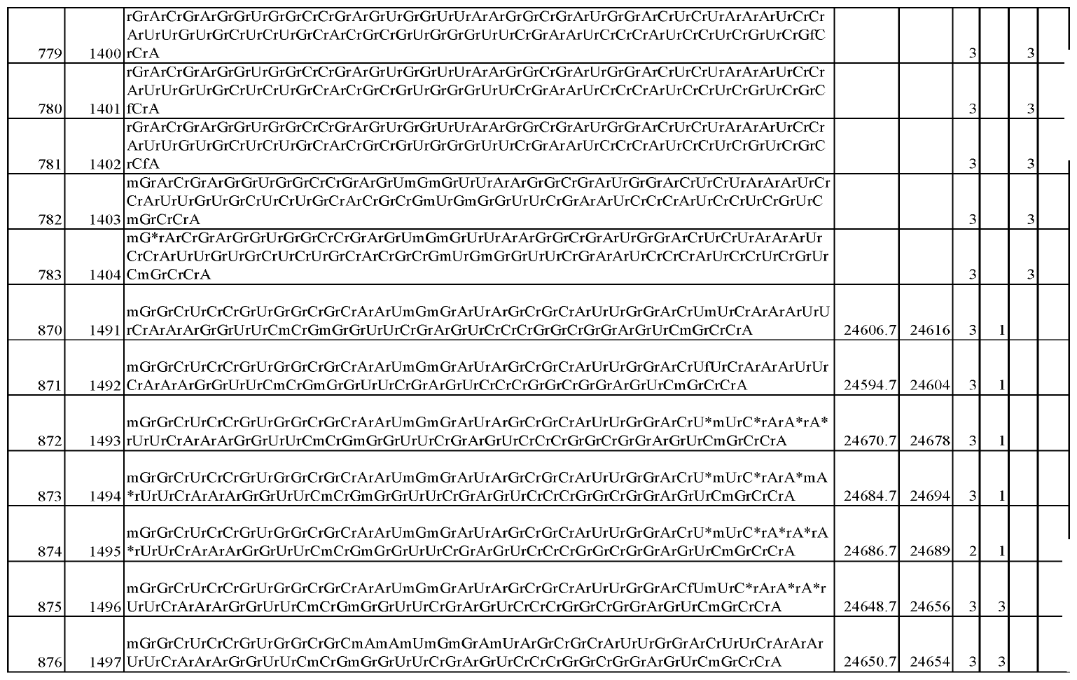

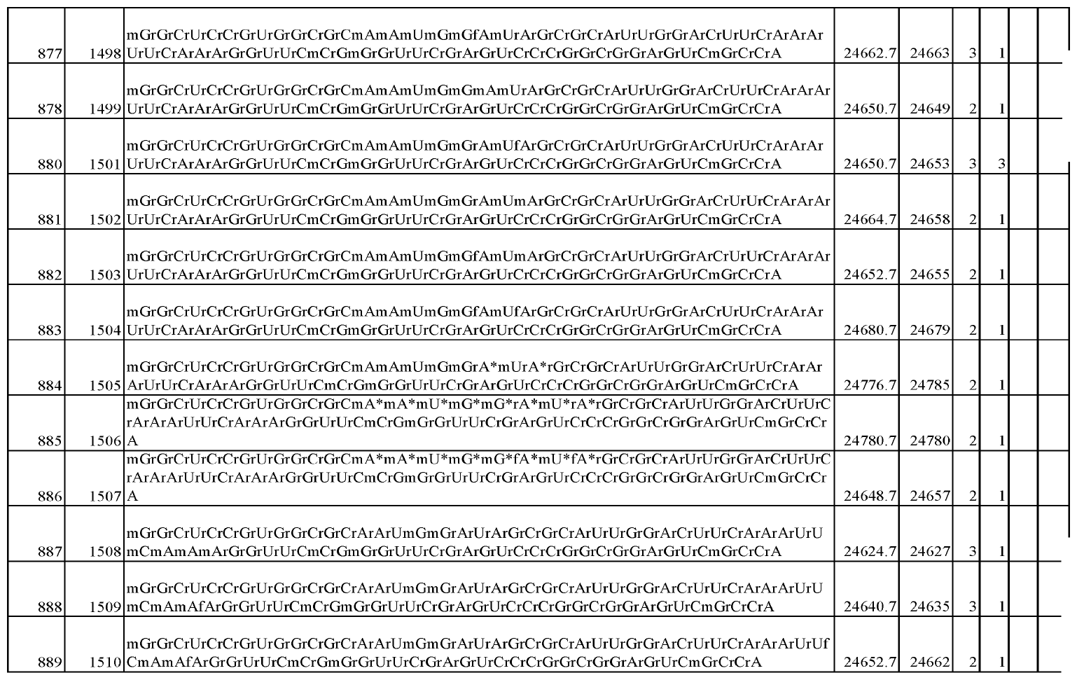

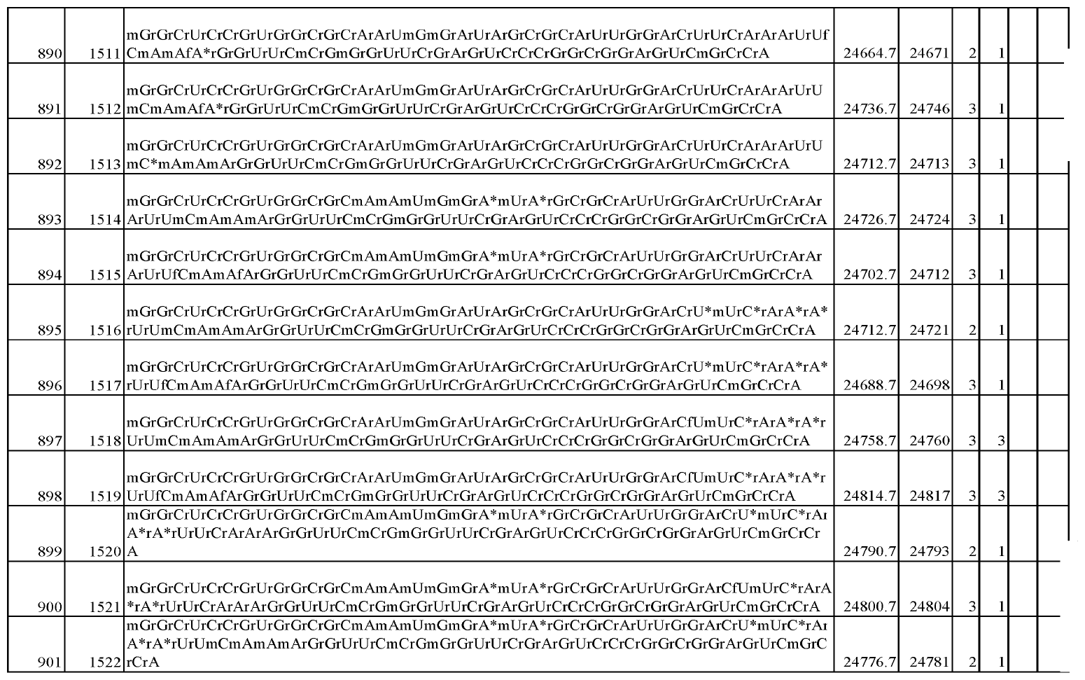

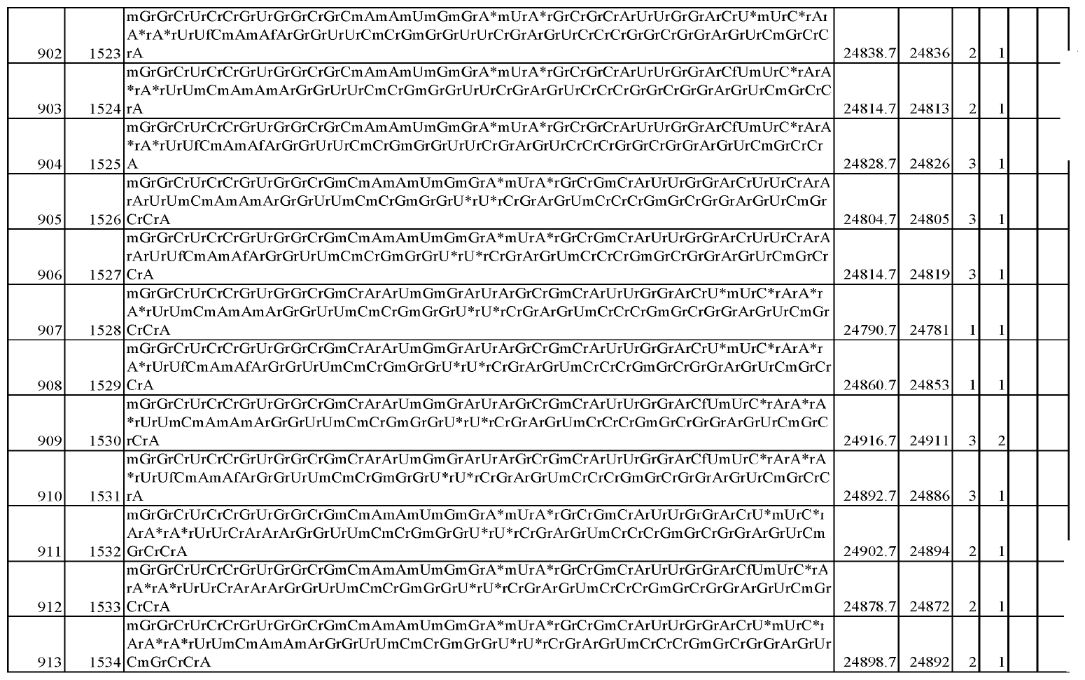

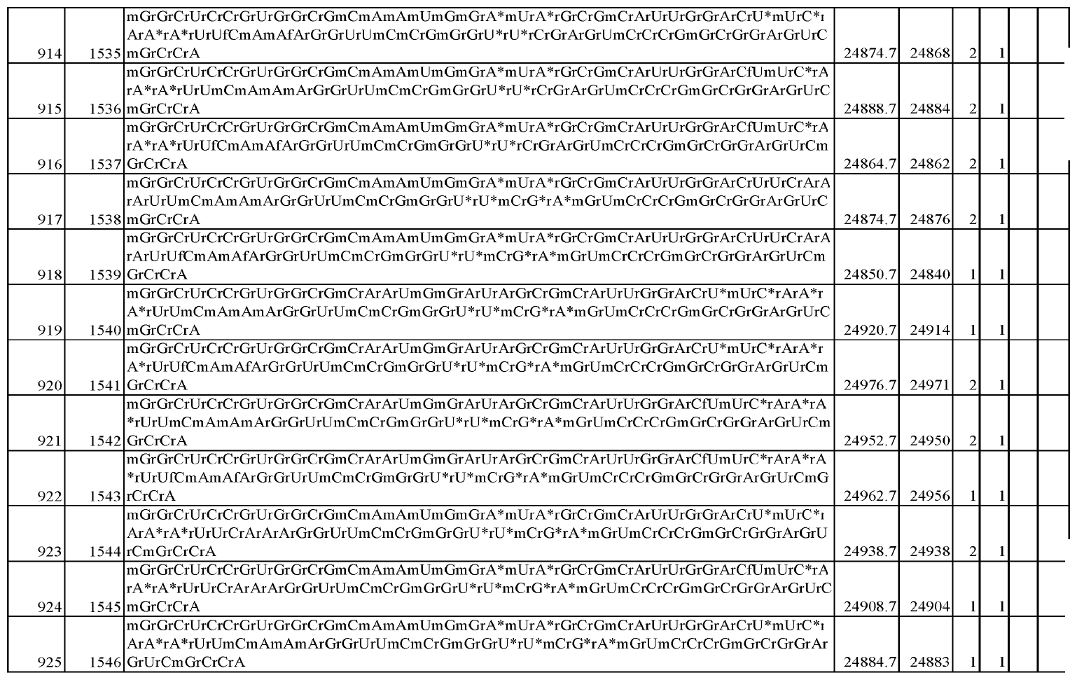

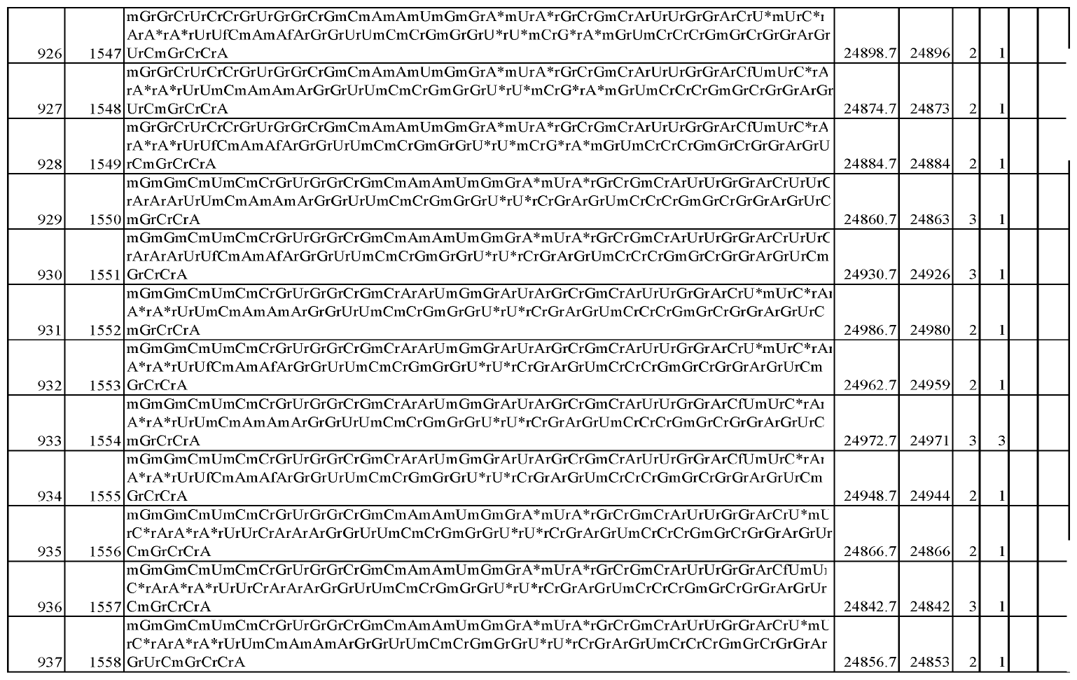

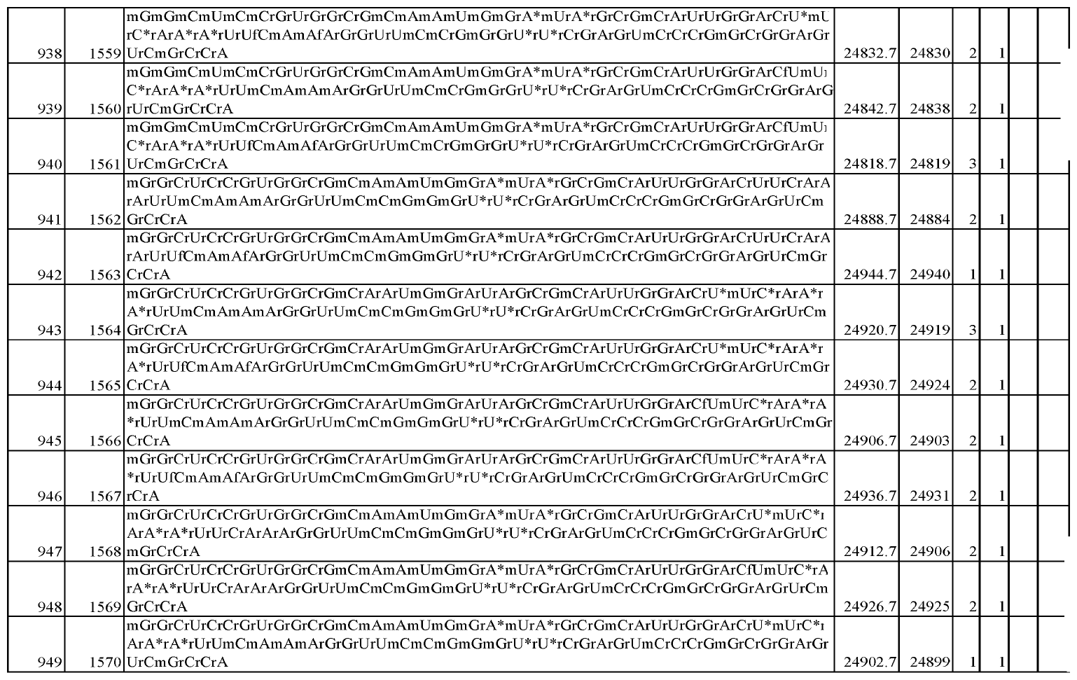

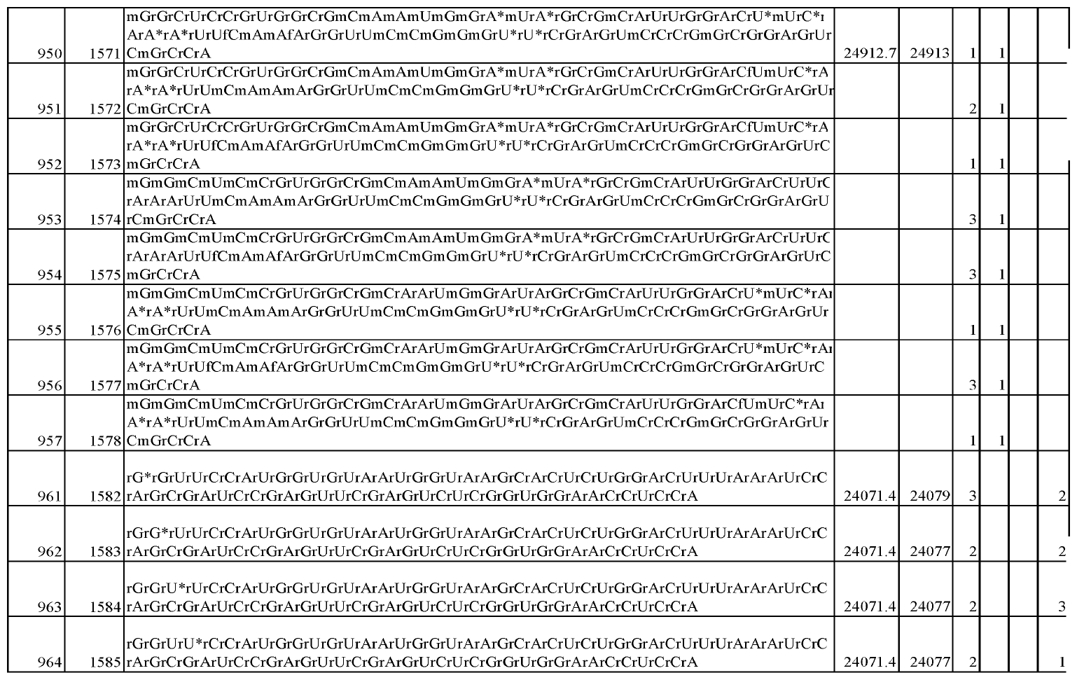

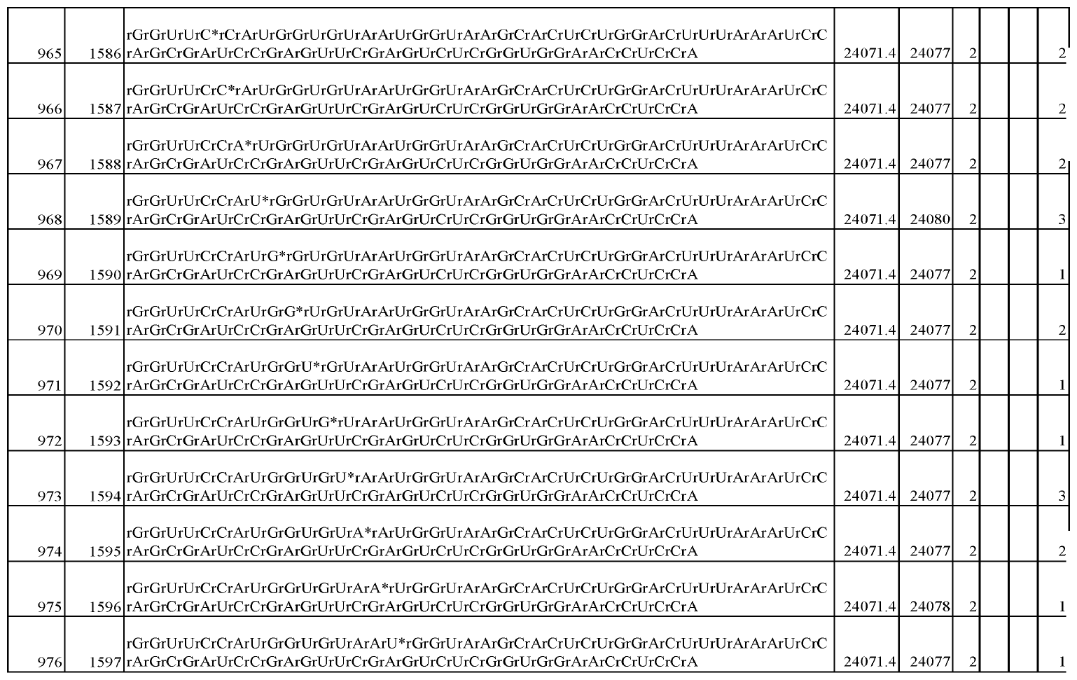

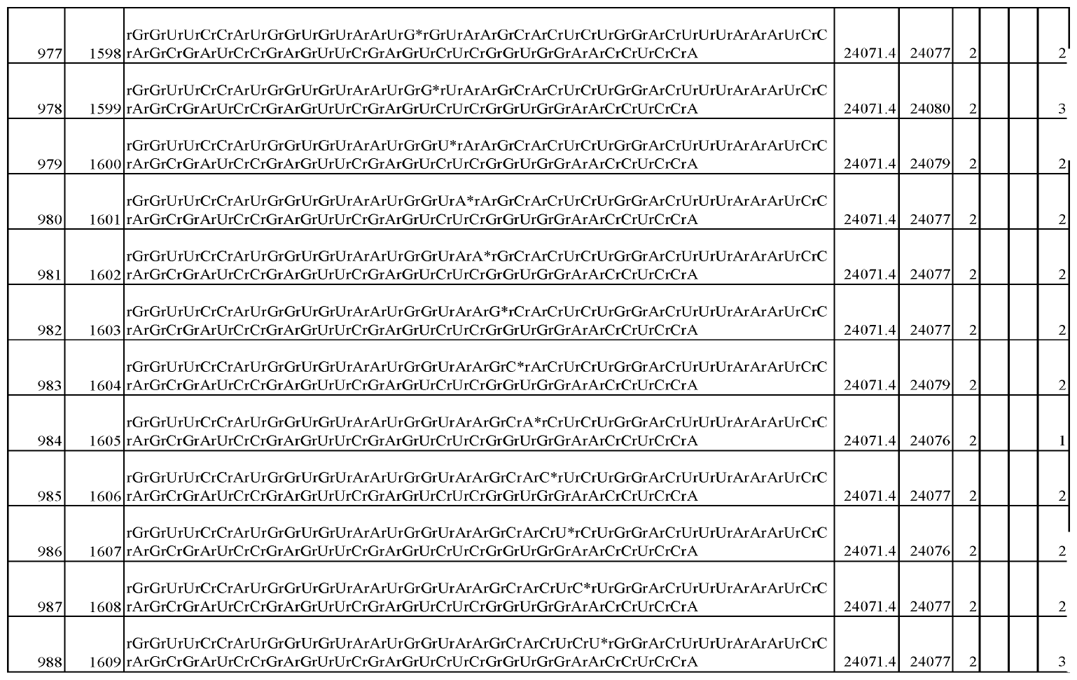

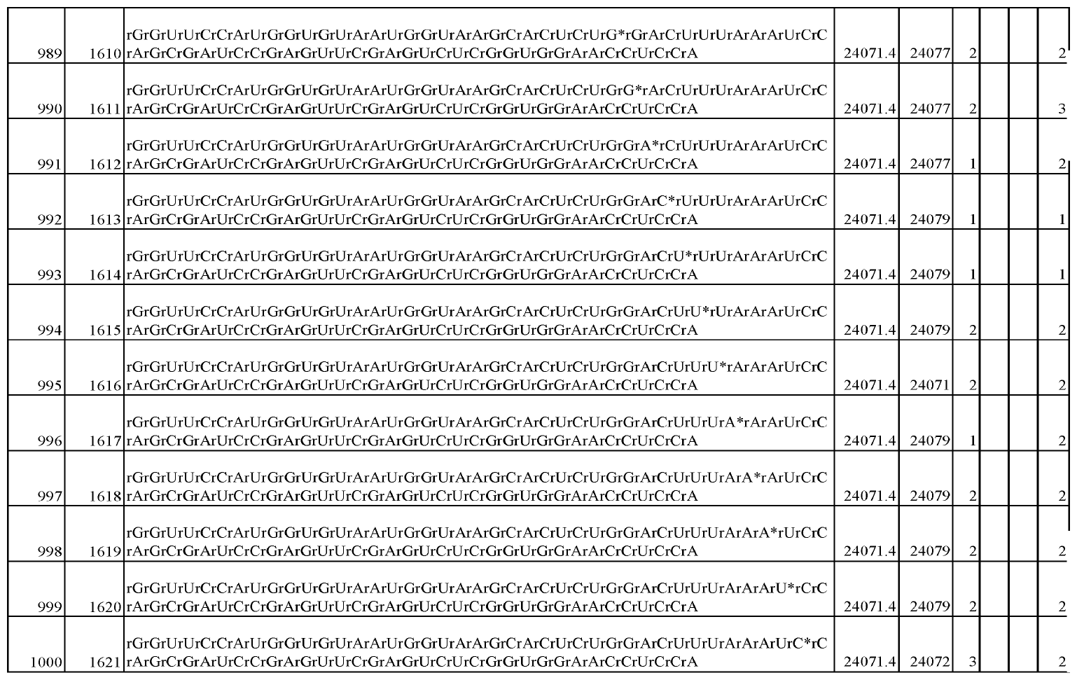

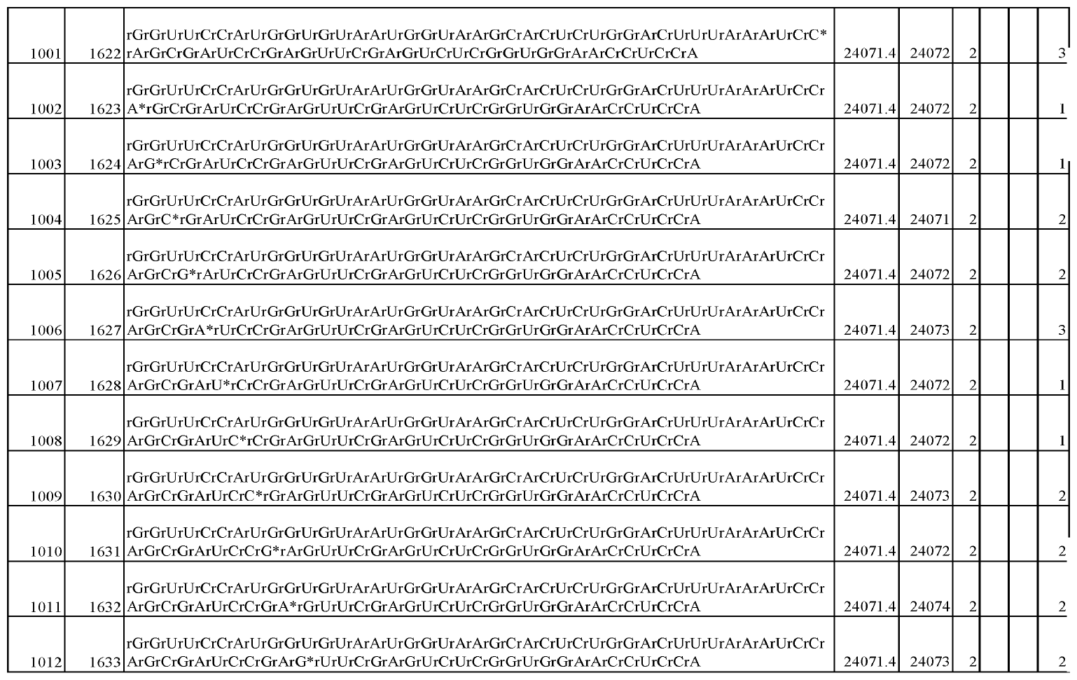

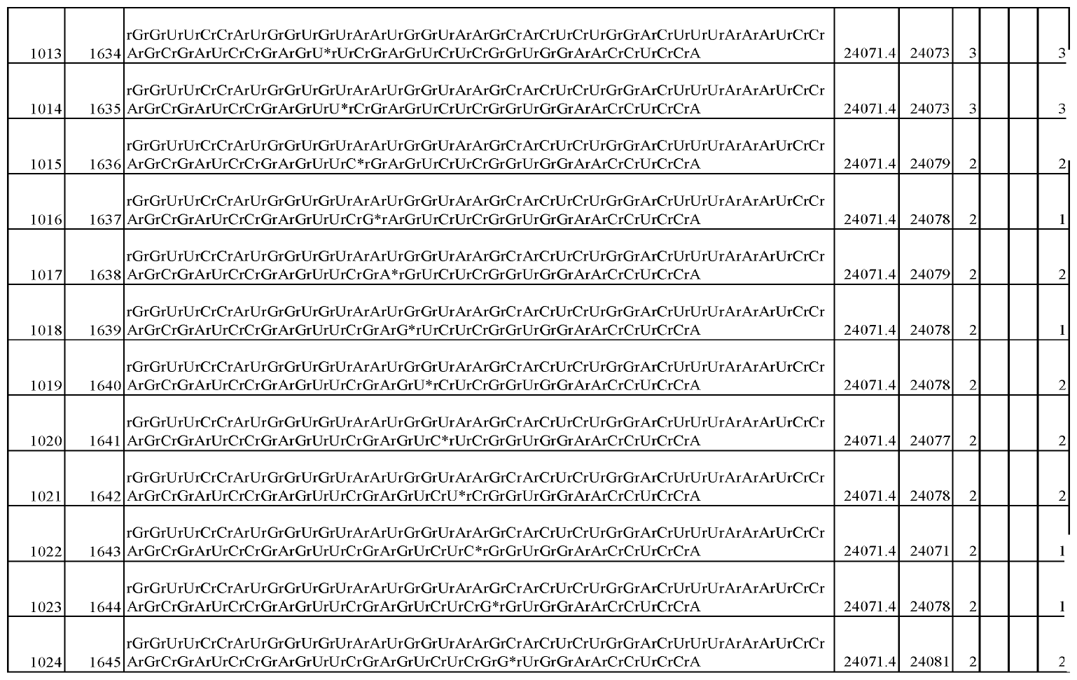

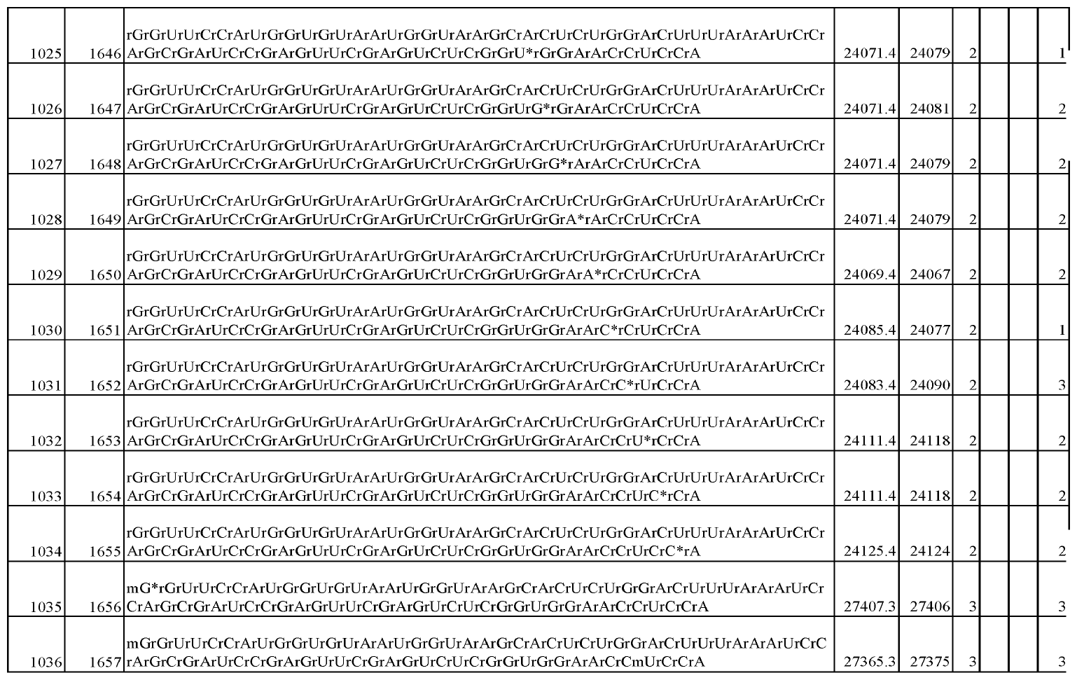

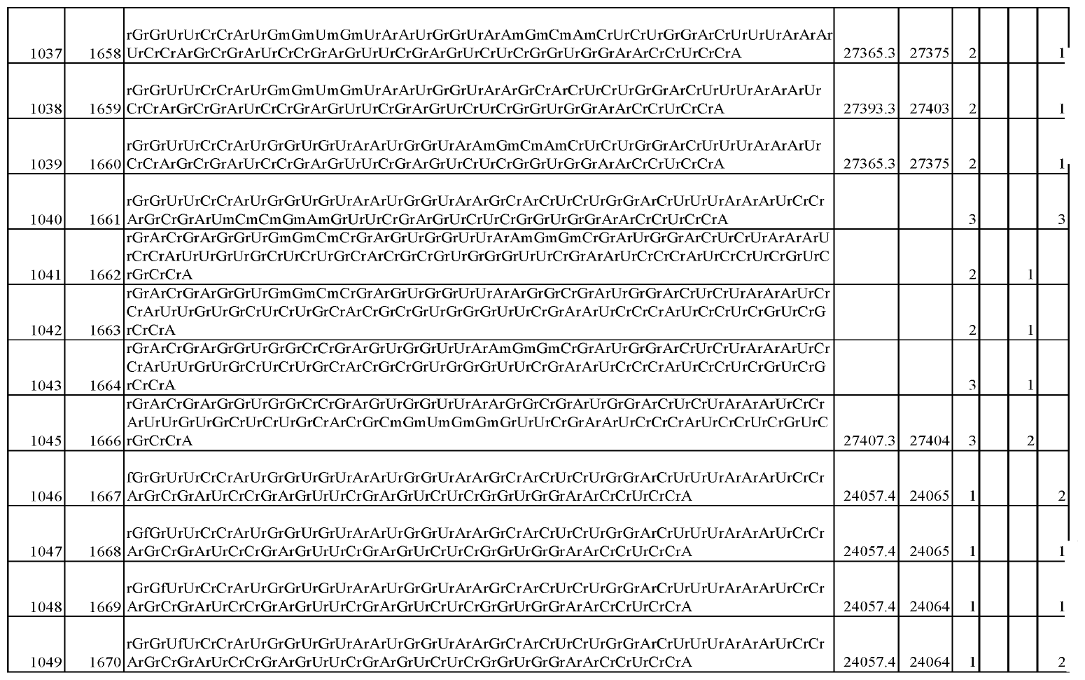

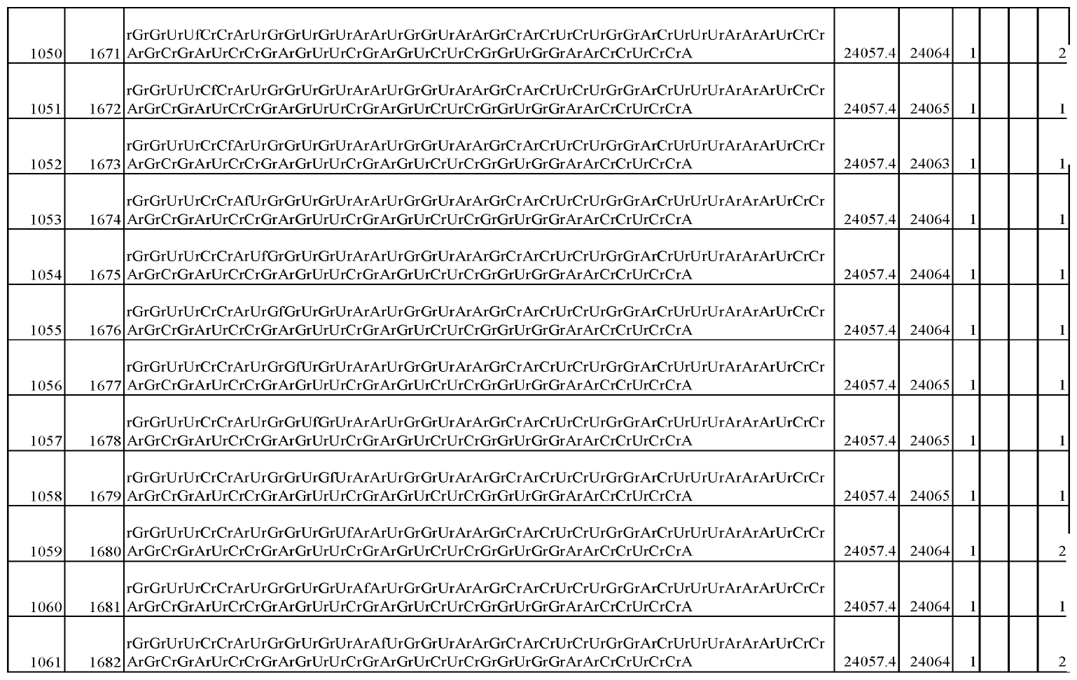

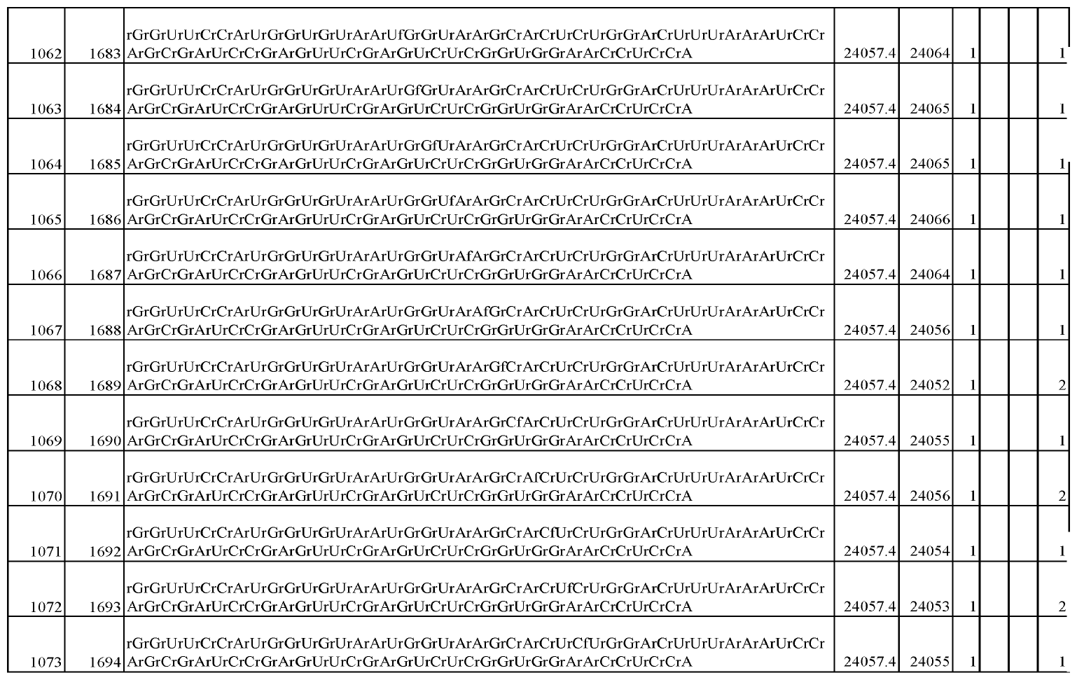

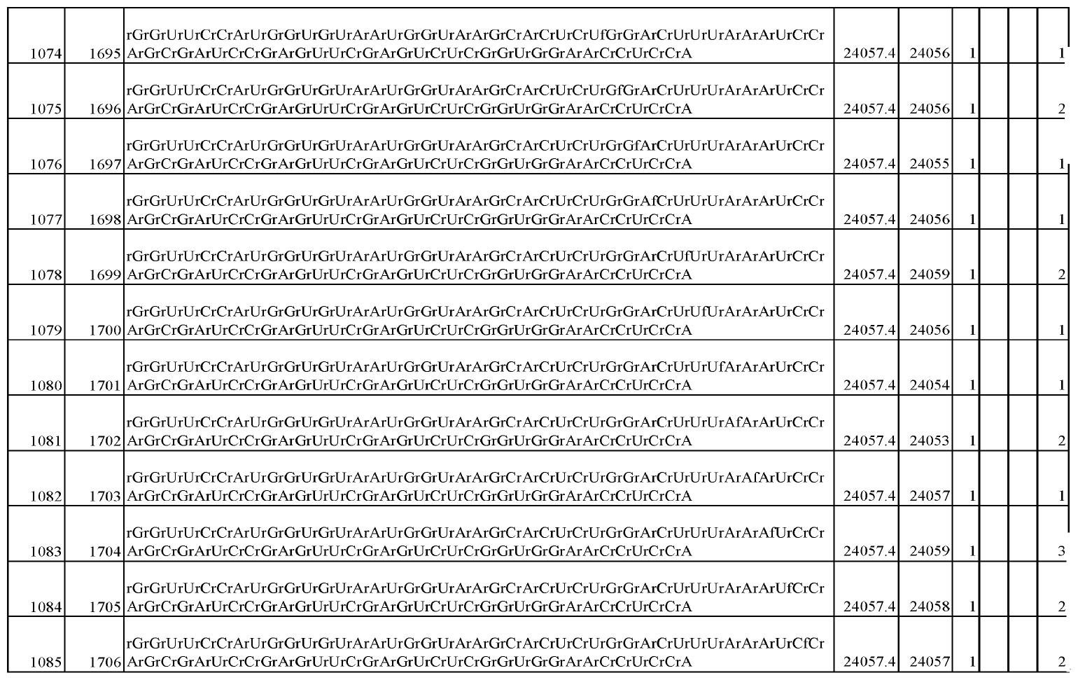

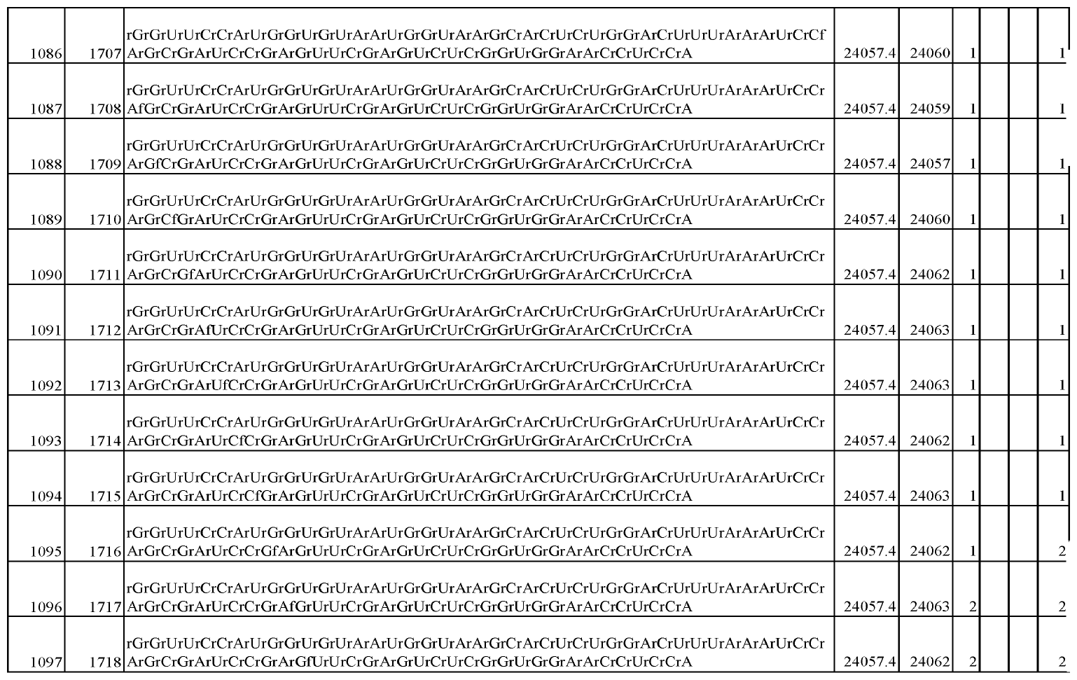

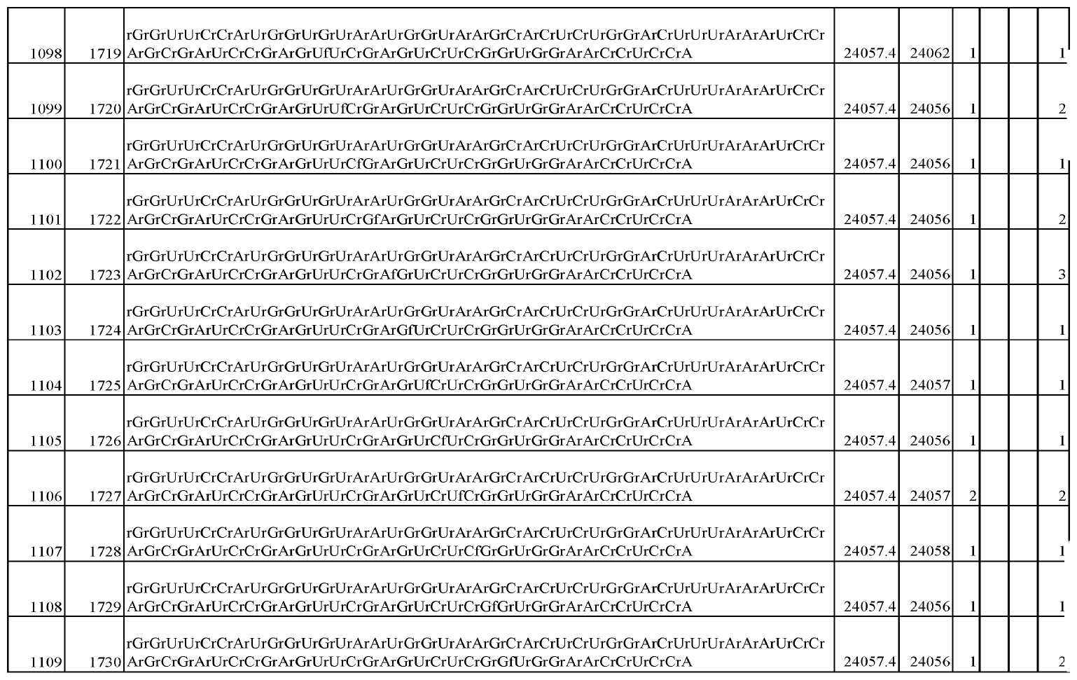

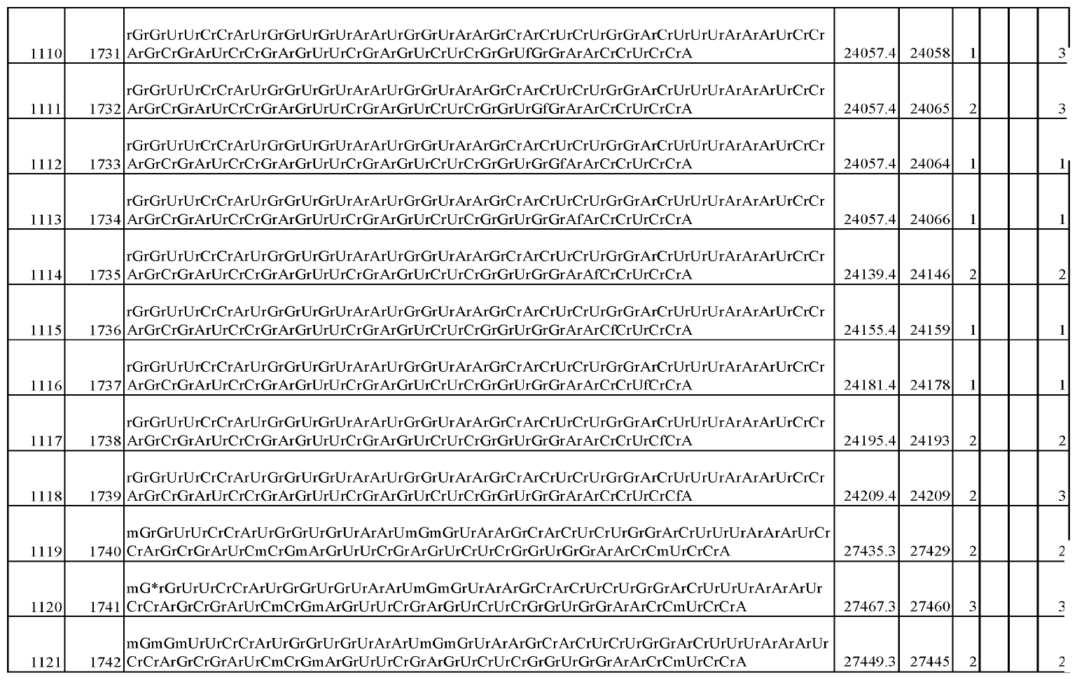

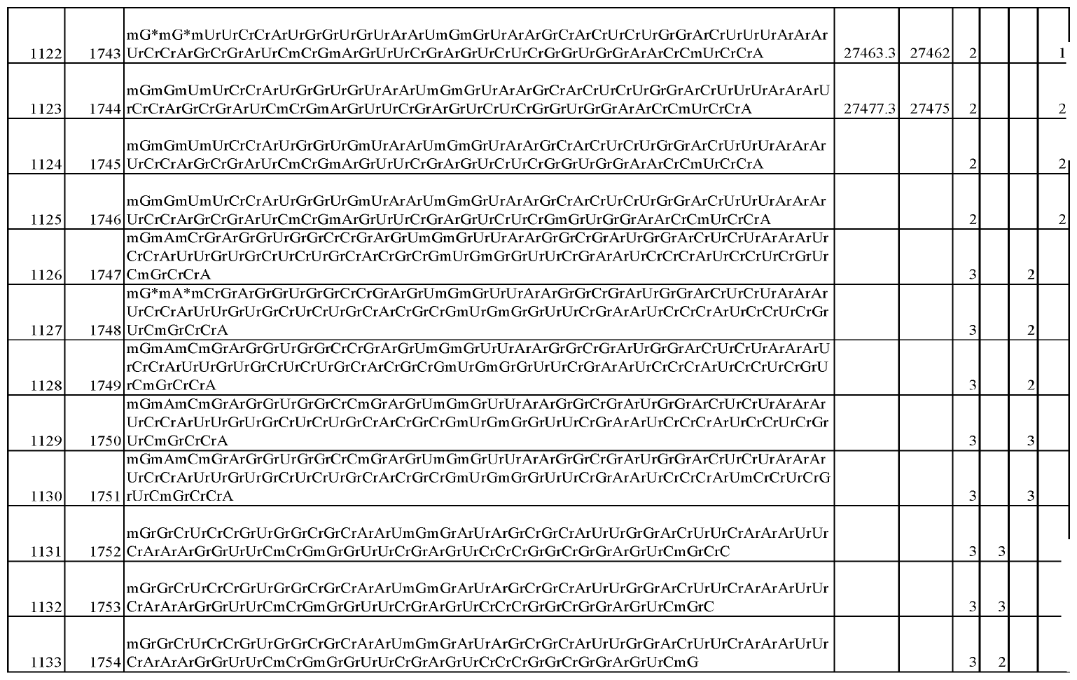

### ENUMERATED EMBODIMENTS

1. A tRNA effector molecule (TREM) comprising a sequence of Formula (I):
[L1]-[ASt Domain1]-[L2]-[DH Domain]-[L3]-[ACH Domain]-[VL Domain]-[TH Domain]-[L4]-[ASt Domain2] (I),
wherein:
independently, [L1] and [VL Domain], are optional; and
a nucleotide within any one of (i) [L1]-[ASt Domain1]-[L2], (ii) [DH Domain]-[L3]; (iii) [ACH Domain]; (iv) [VL Domain]; (v) [TH Domain]; and [L4]-[ASt Domain2] comprises a nucleotide having a non-naturally occurring modification.

2. The TREM of embodiment 1, wherein the non-naturally occurring modification is present on the 2′-position of a nucleotide sugar or within the internucleotide region (e.g., a backbone modification).

3. The TREM of any one of the preceding embodiments, wherein the non-naturally occurring modification is selected from a 2′-O-methyl (2-OMe), 2′-halo (e.g., 2′F or 2′Cl), 2′-O-methoxyethyl (2′MOE), or 2′deoxy modification.

4. The TREM of any one of the preceding embodiments, wherein the non-naturally occurring modification is a 2′OMe modification.

5. The TREM of any one of the preceding embodiments, wherein the non-naturally occurring modification is a 2′halo (e.g., 2′F or 2′Cl) modification.

6. The TREM of any one of the preceding embodiments, wherein the non-naturally occurring modification is a 2′MOE modification.

7. The TREM of any one of the preceding embodiments, wherein the non-naturally occurring modification is a 2′-deoxy modification.

8. The TREM of any one of the preceding embodiments, wherein the non-naturally occurring modification is present in the internucleotide region (e.g., a backbone modification).

9. The TREM of embodiment 8, wherein the non-naturally occurring modification is a phosphorothioate modification.

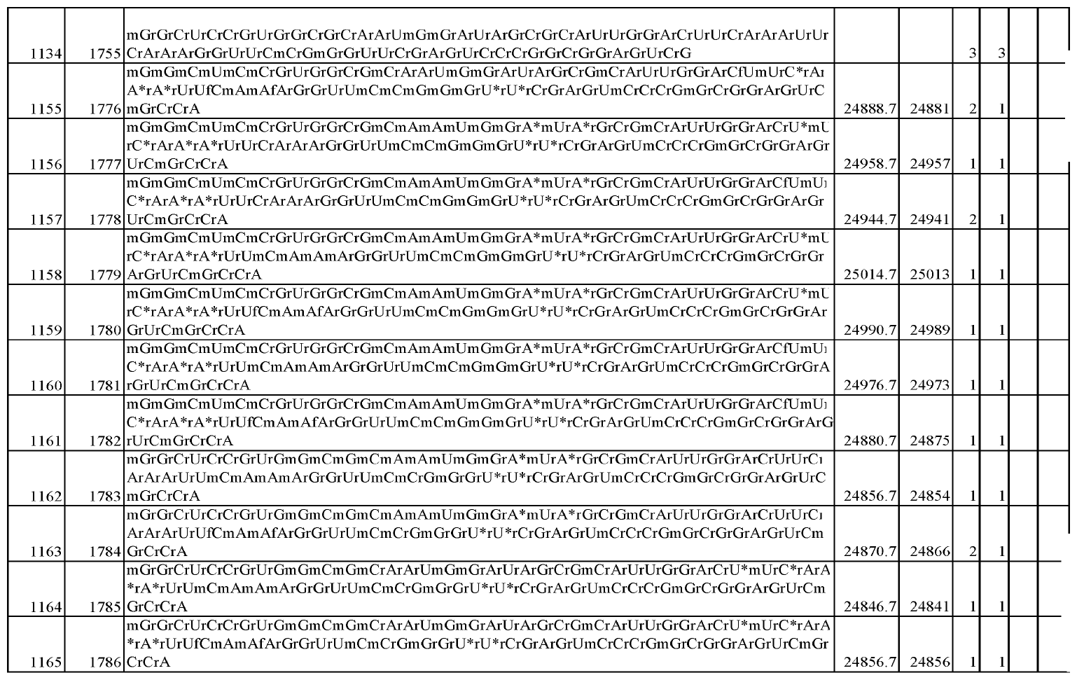

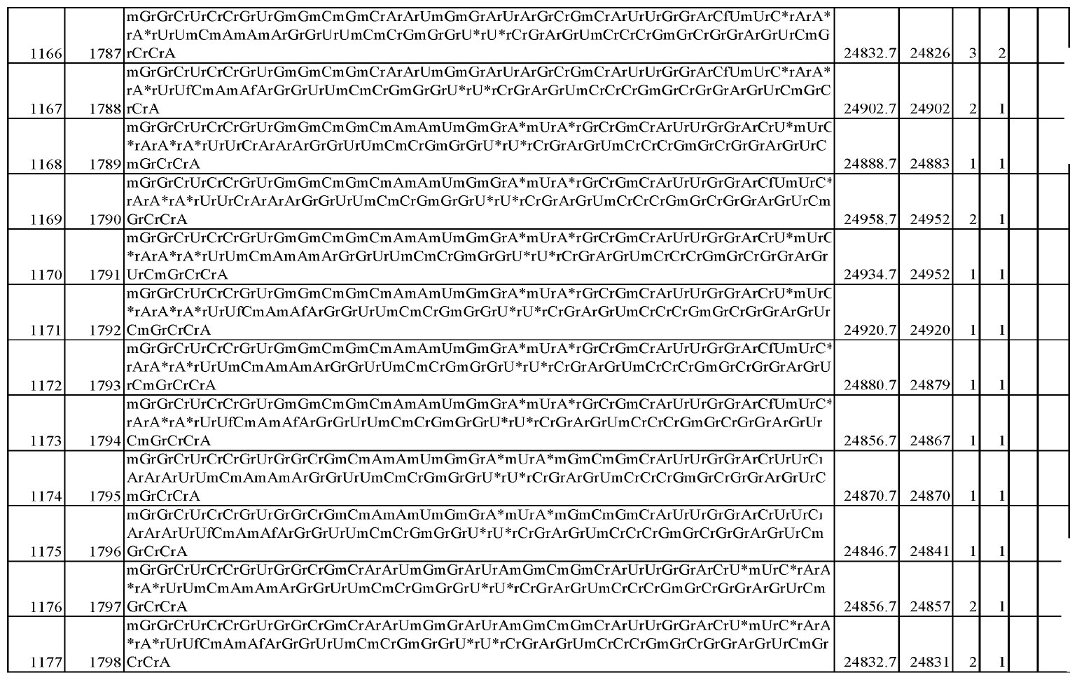

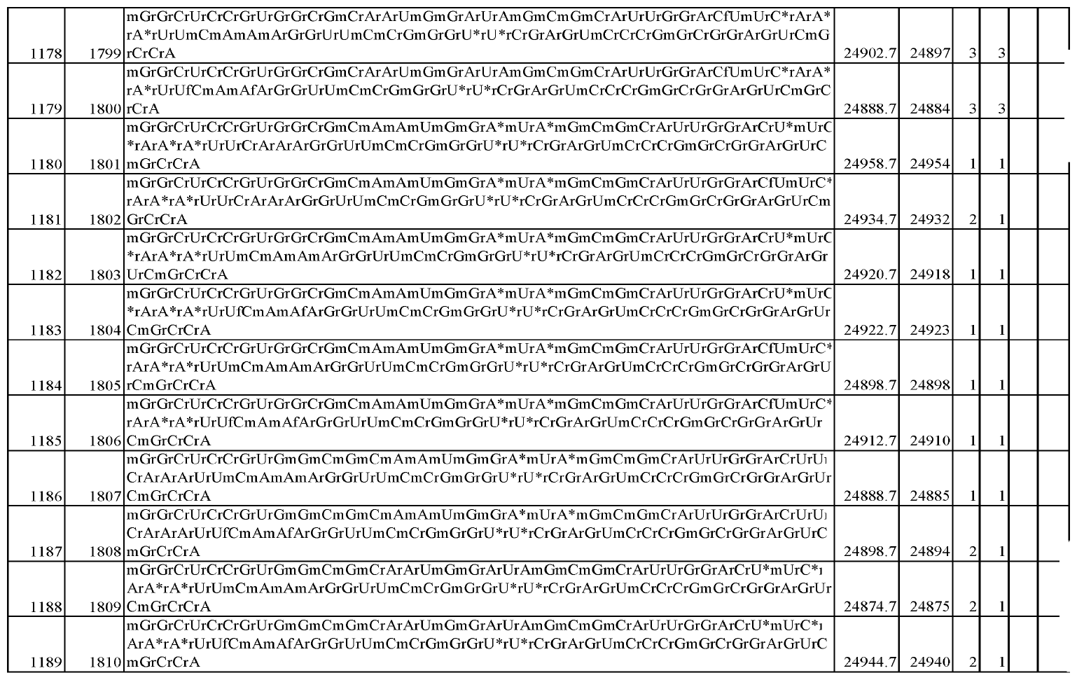

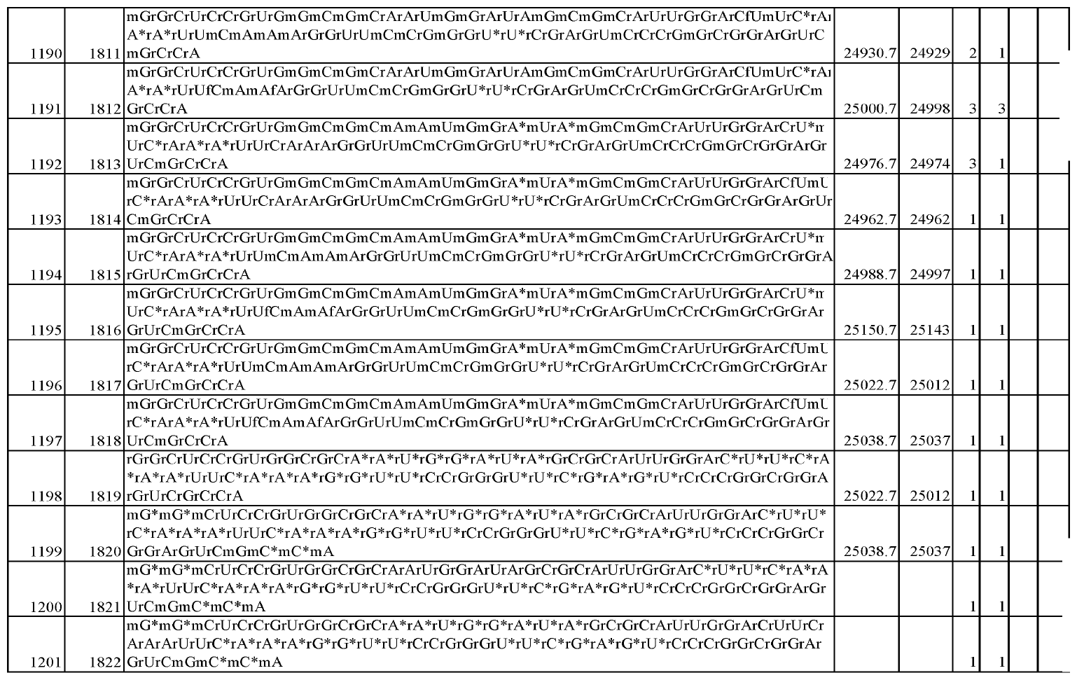

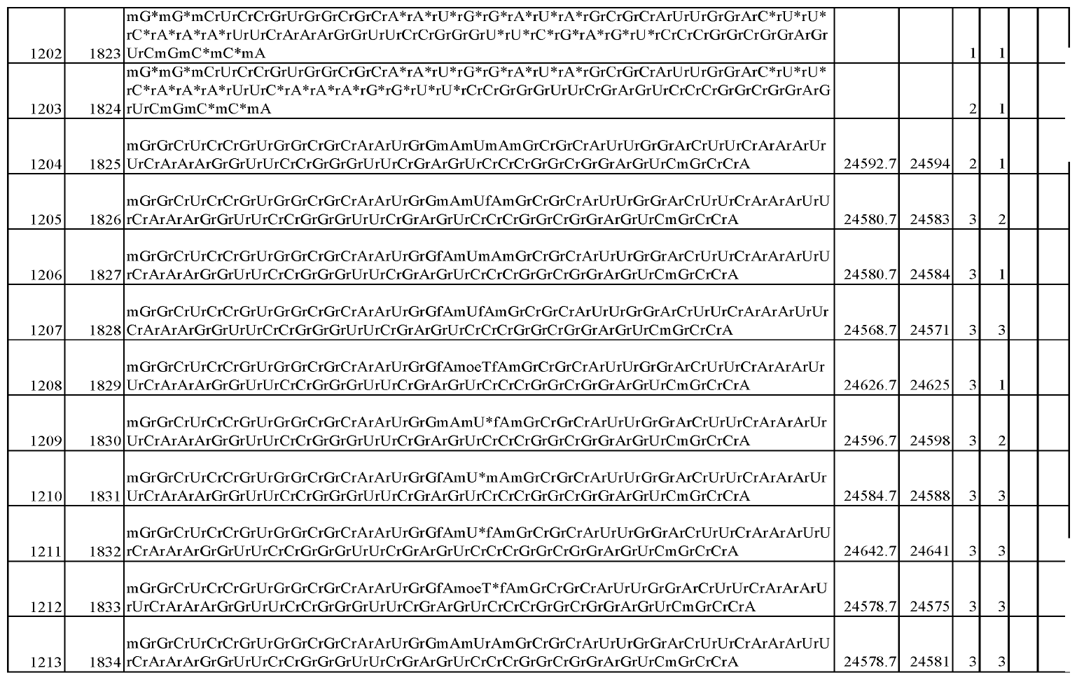

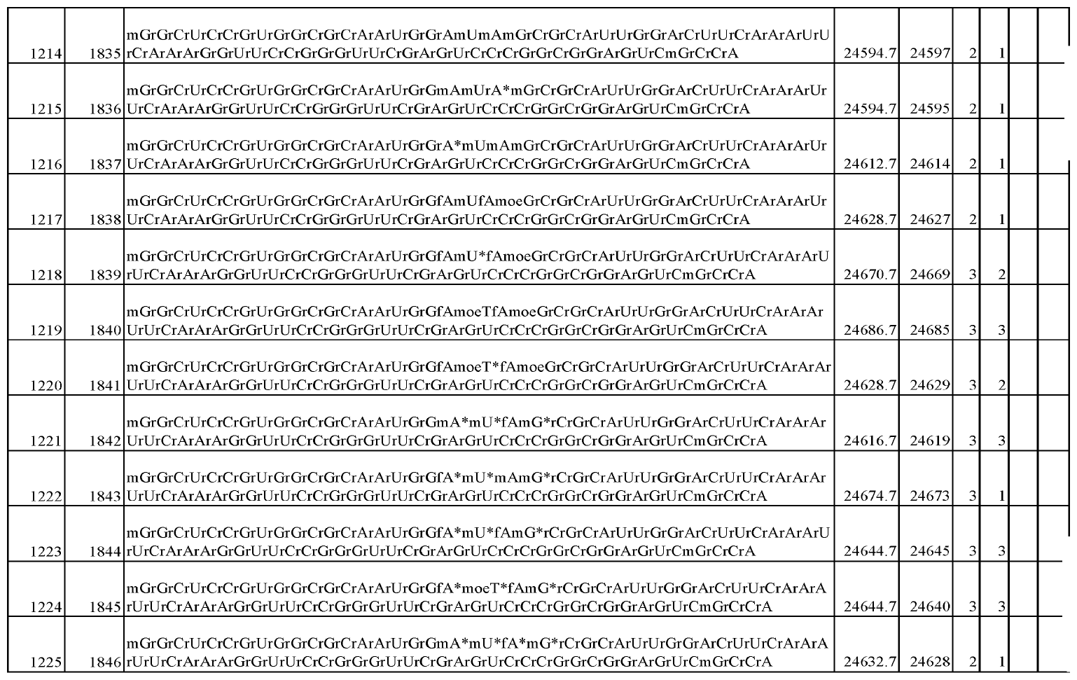

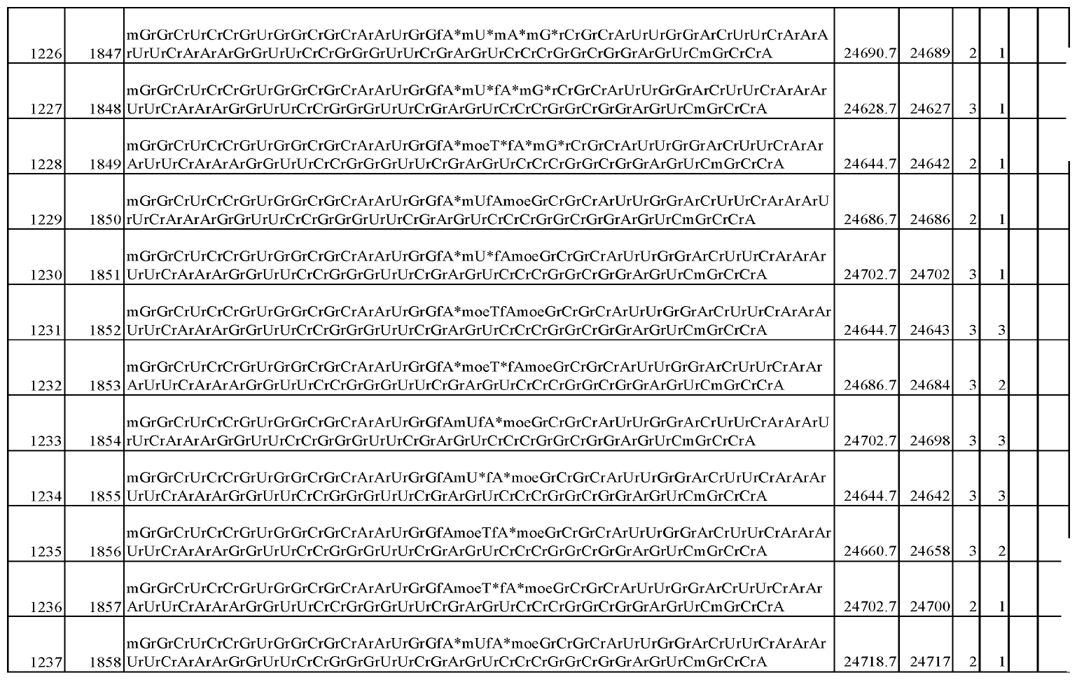

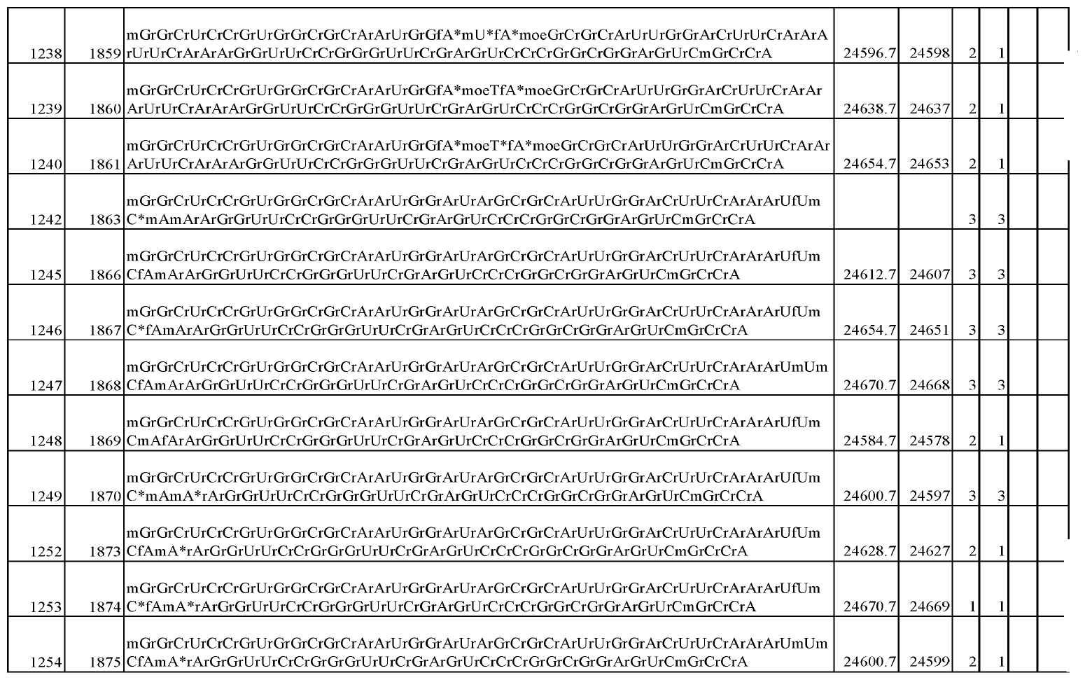

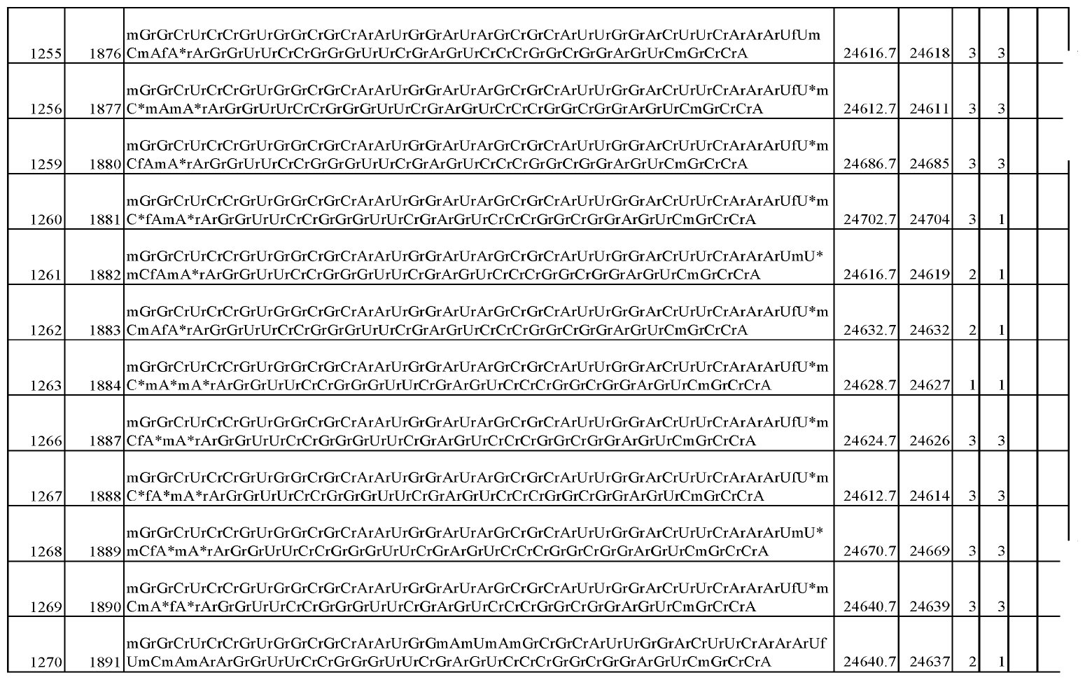

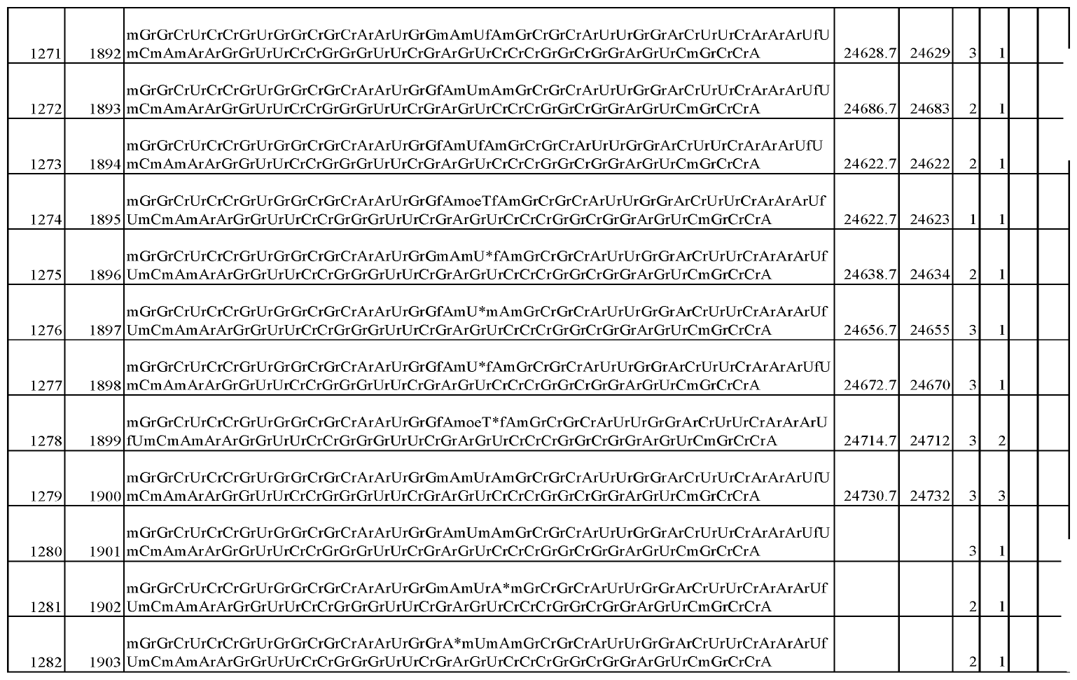

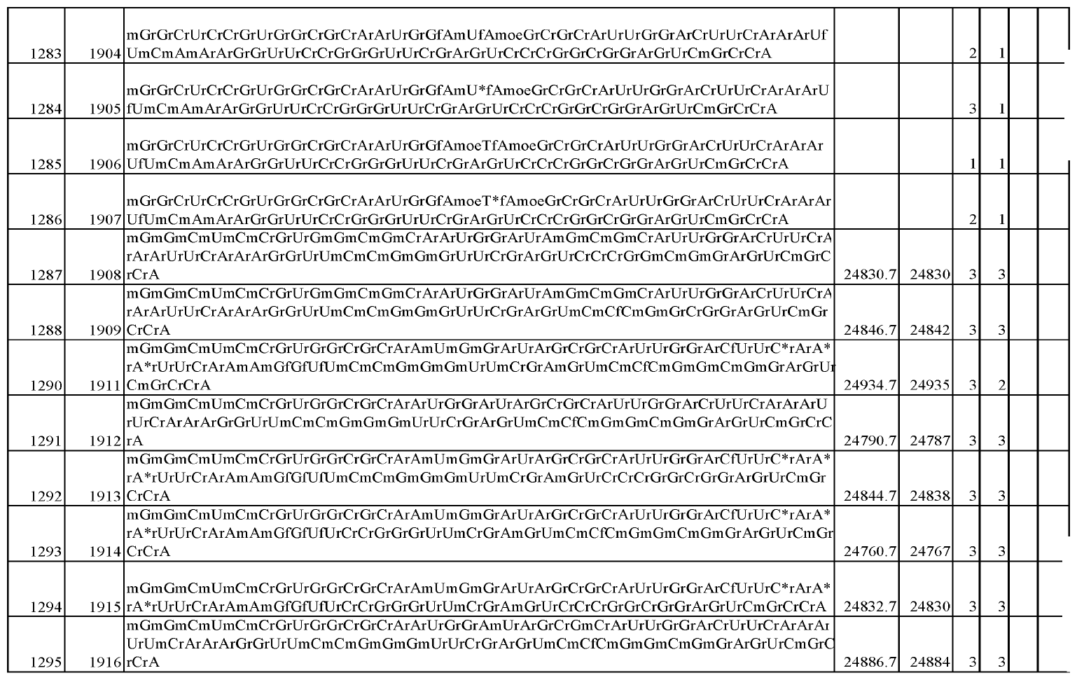

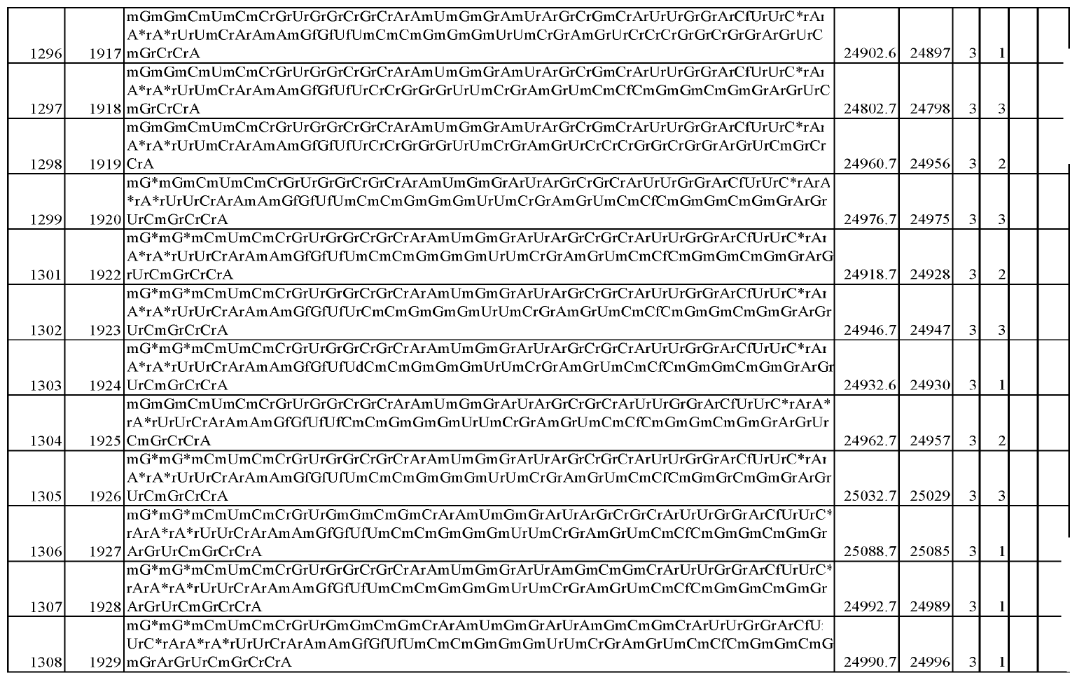

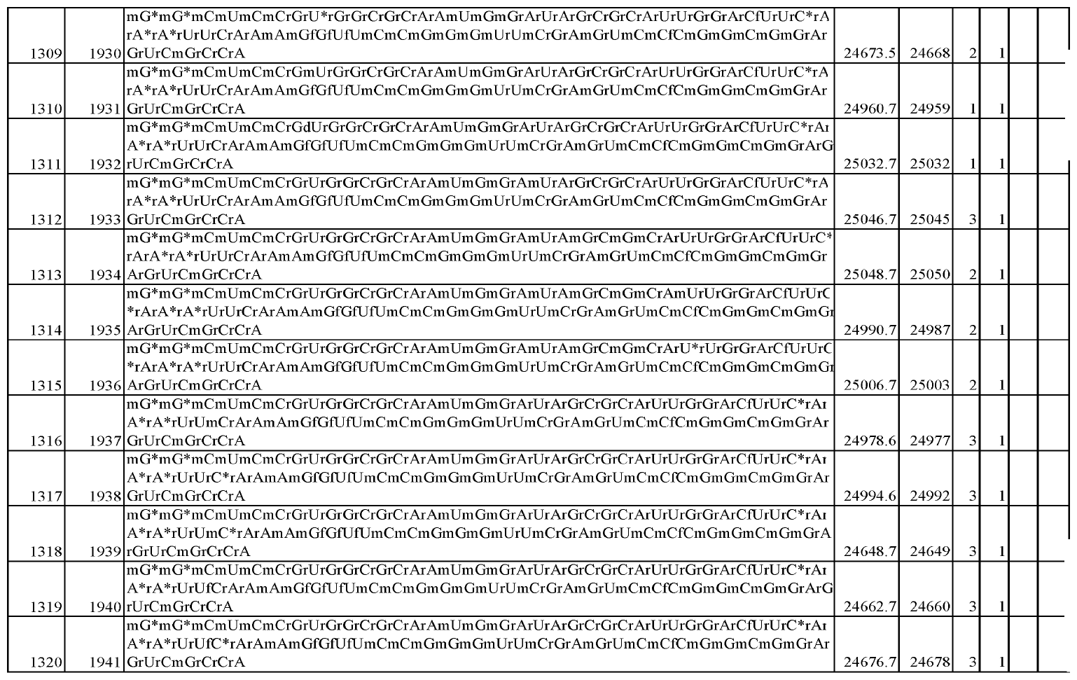

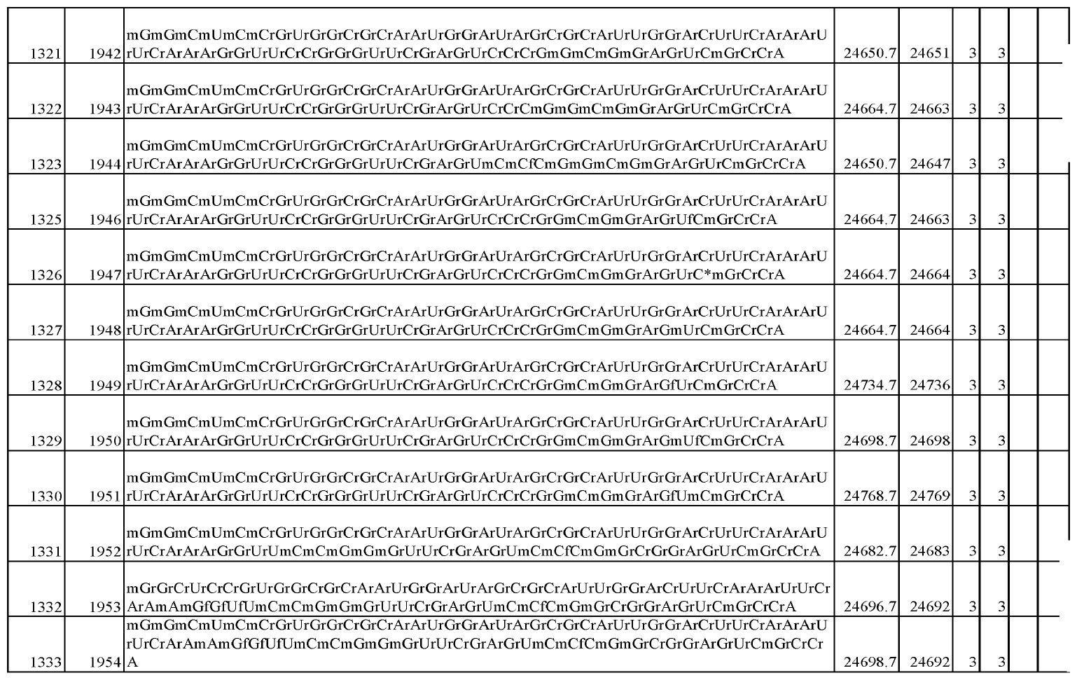

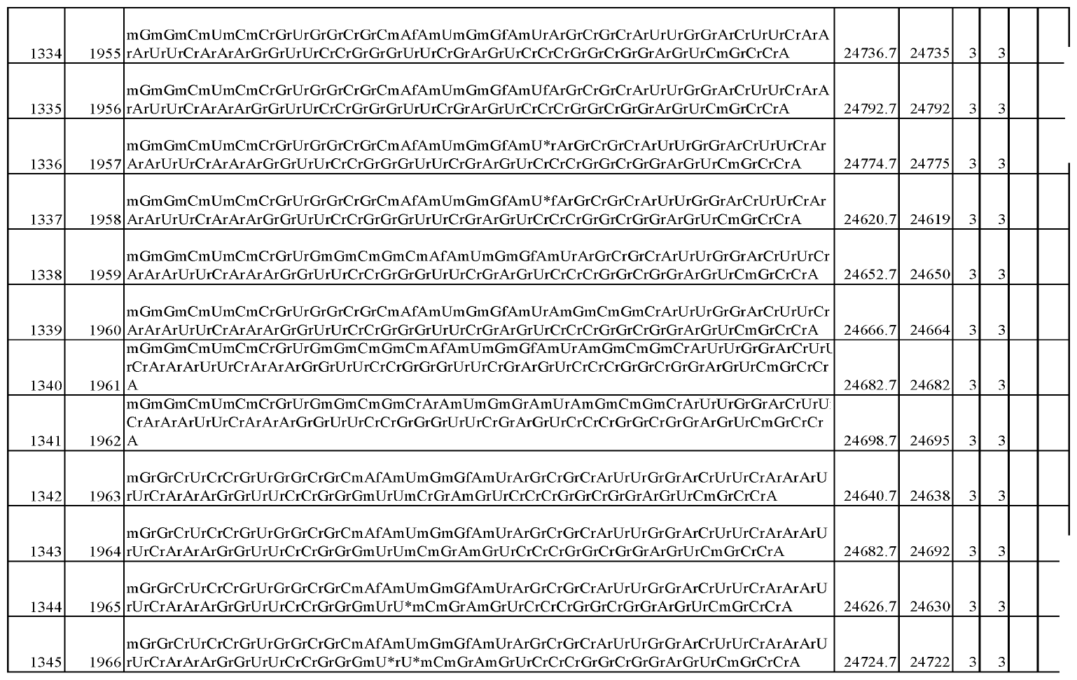

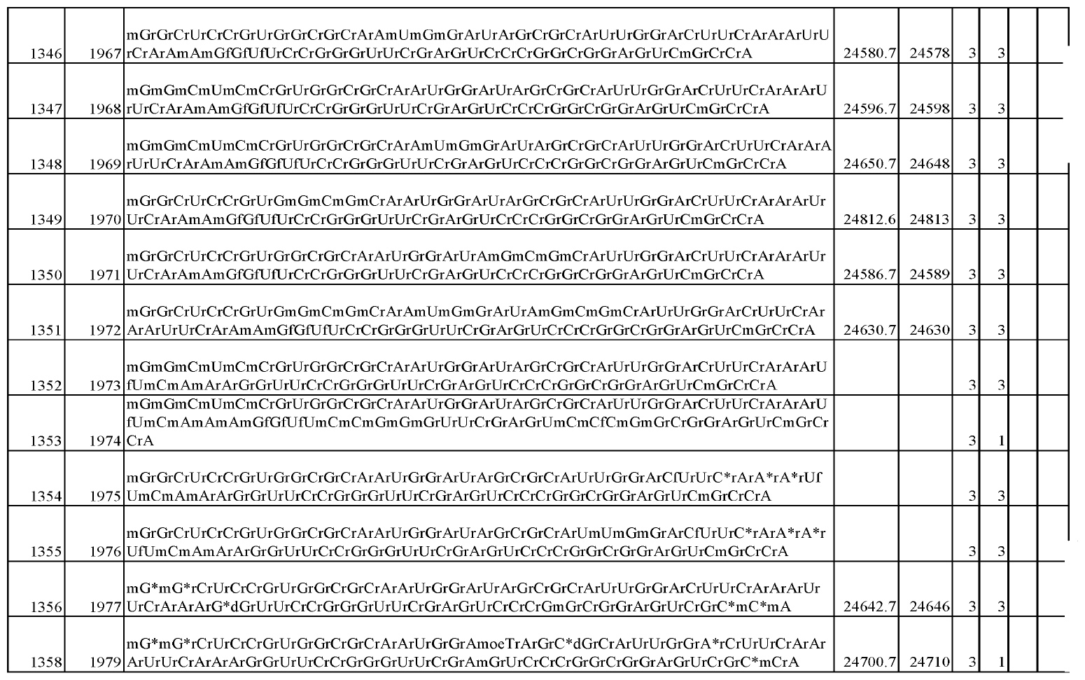

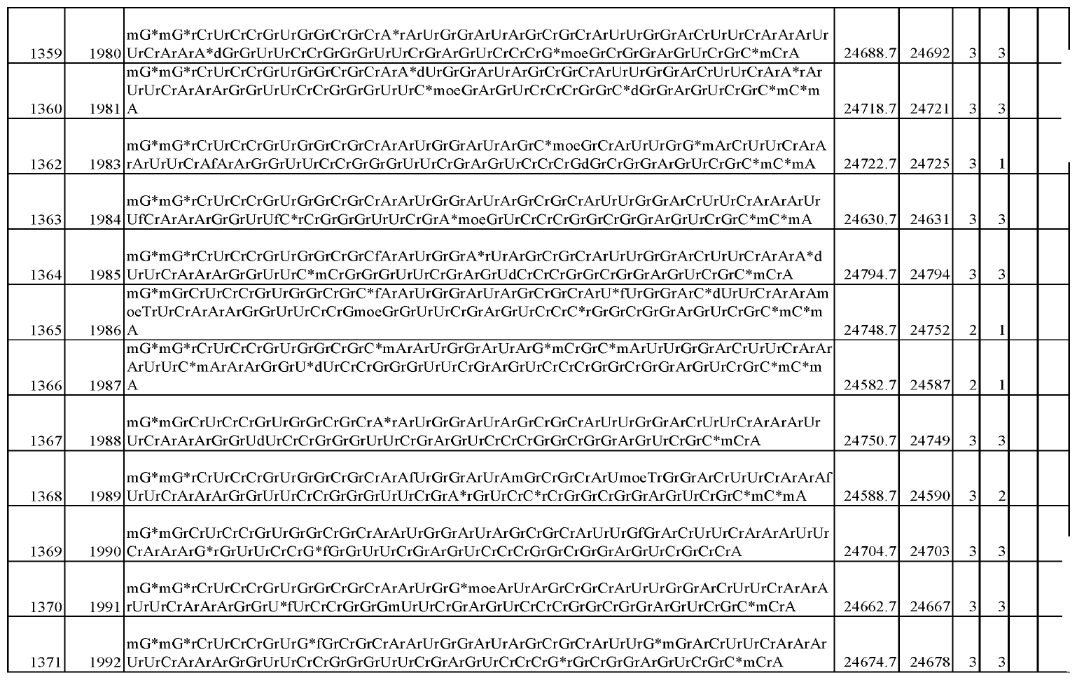

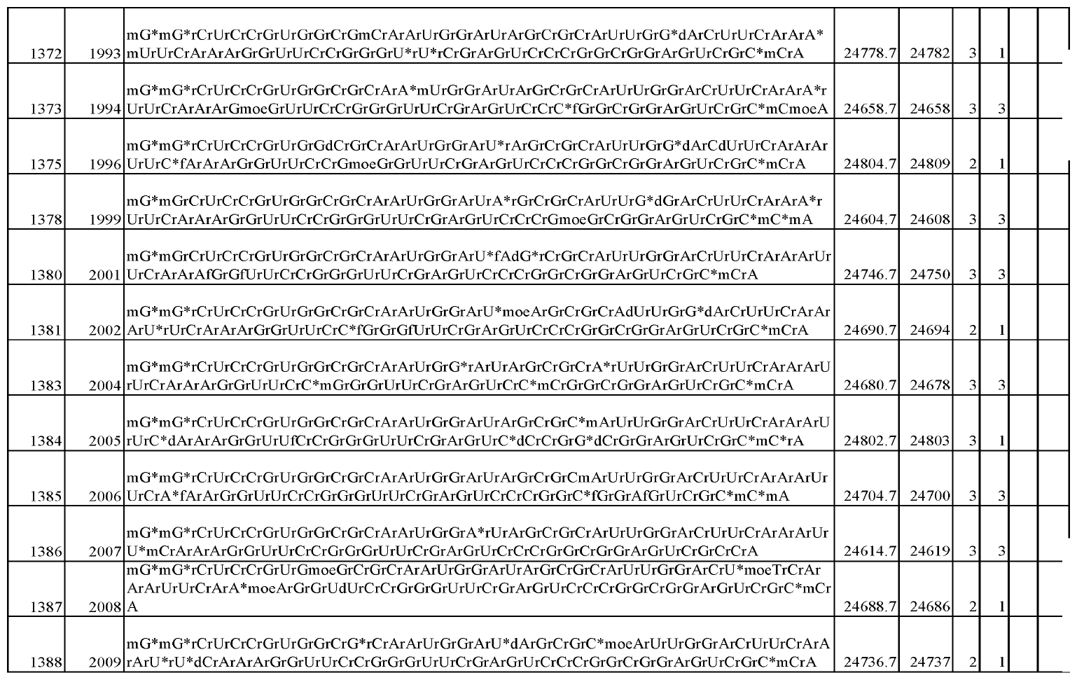

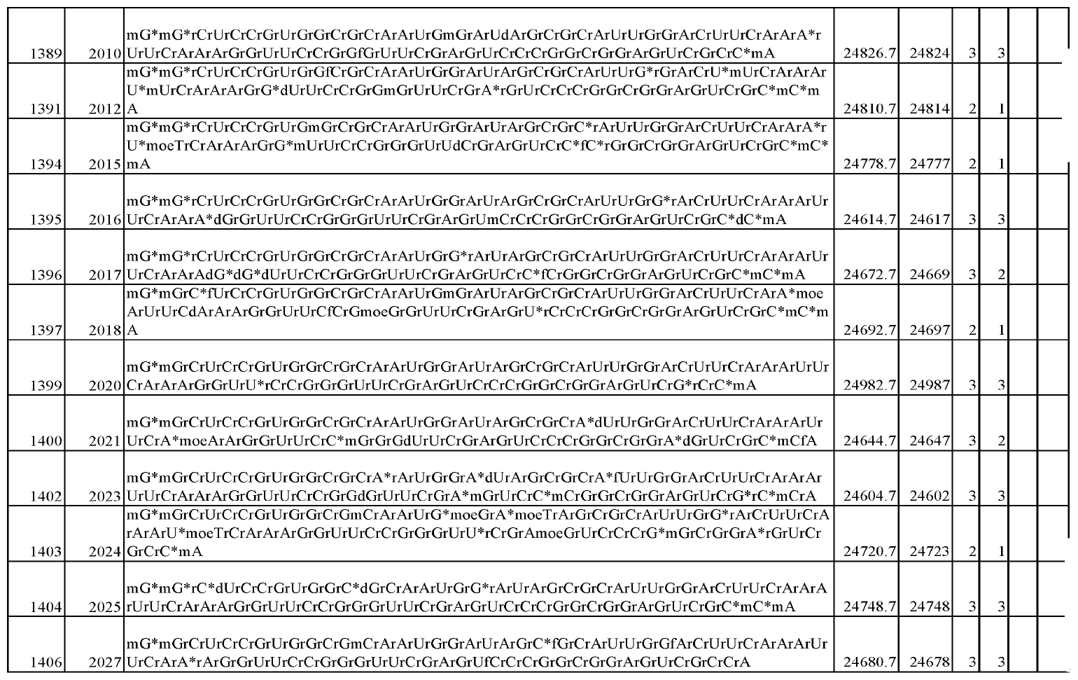

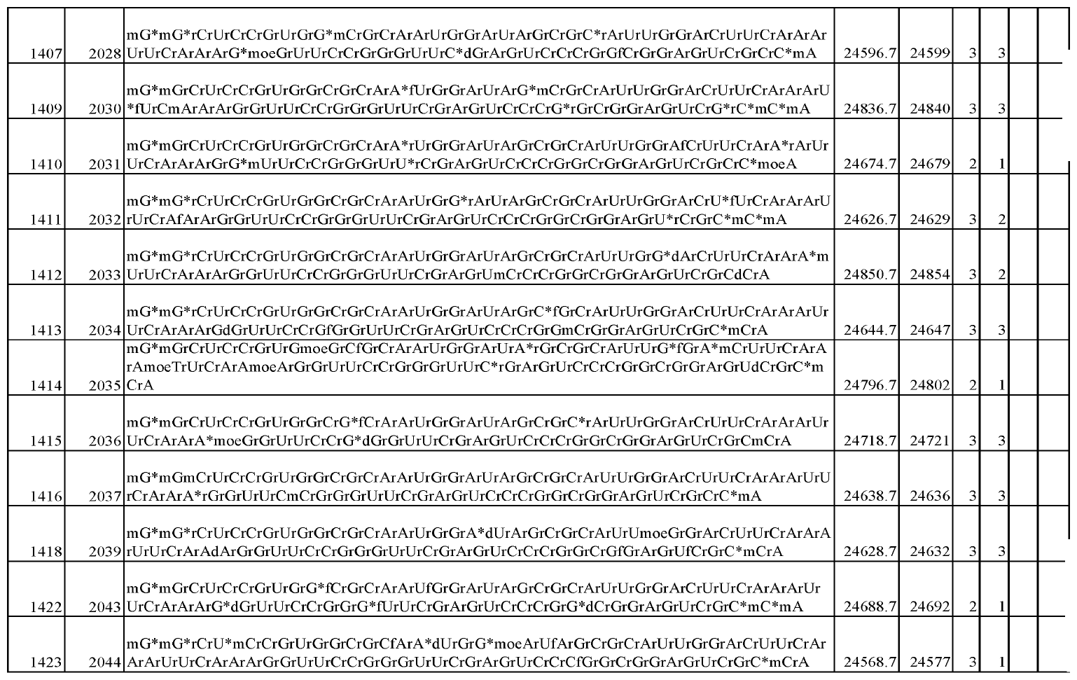

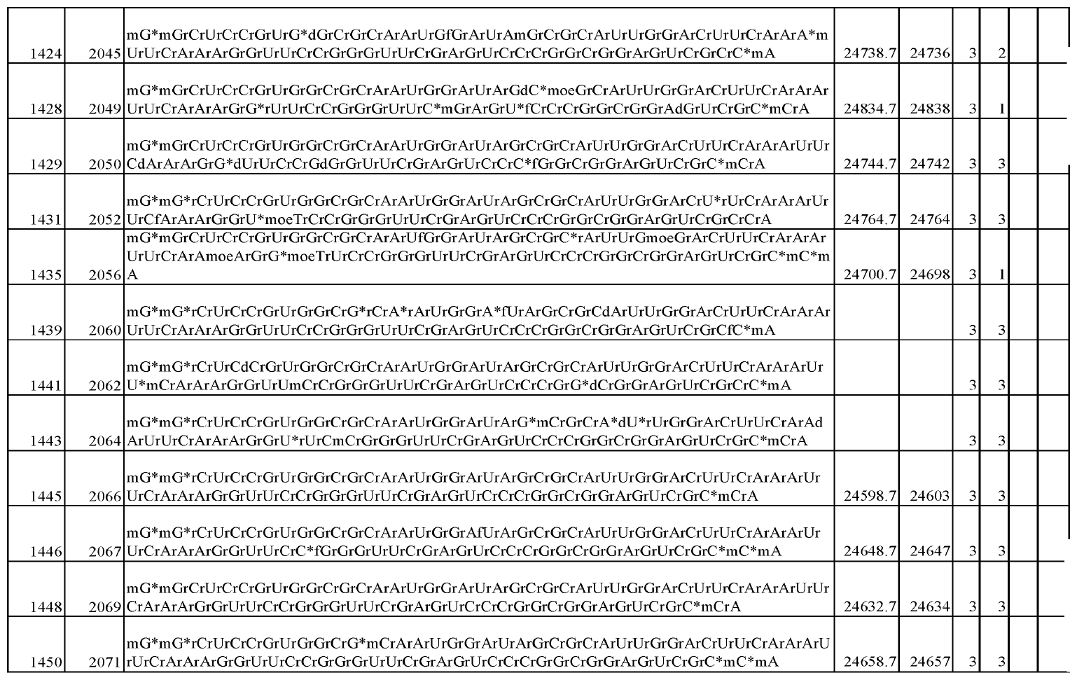

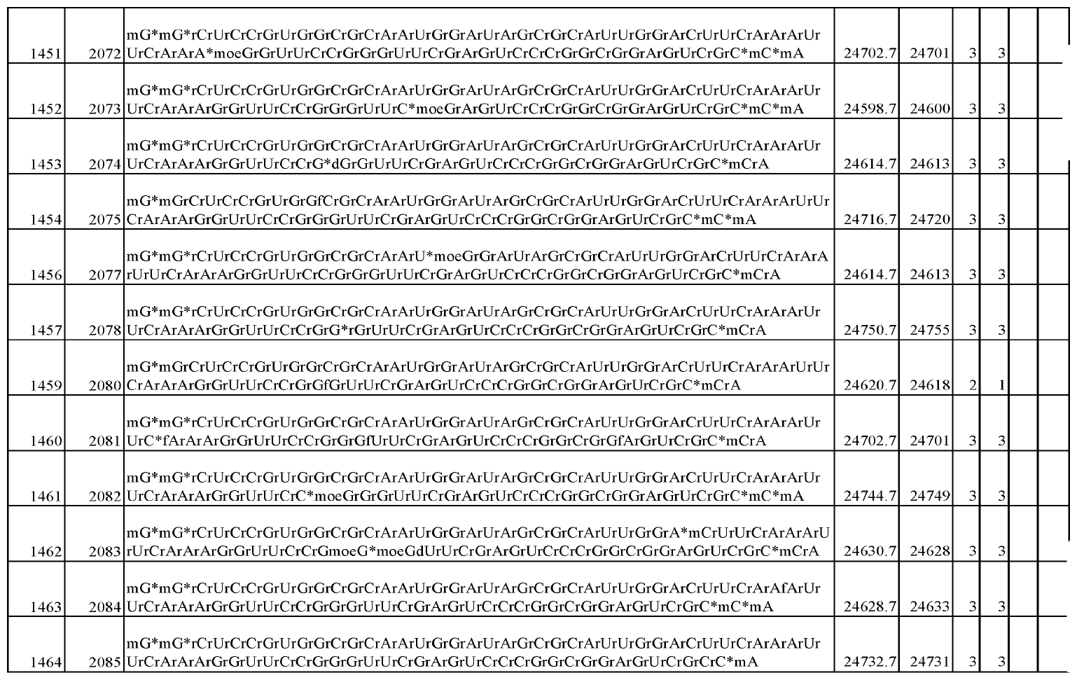

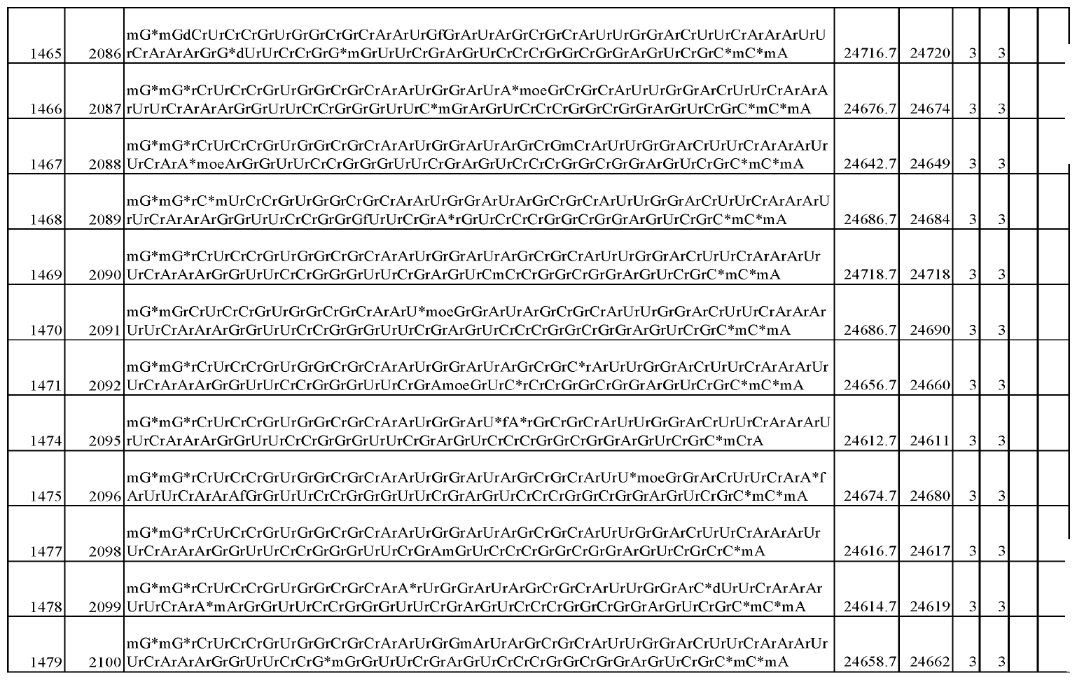

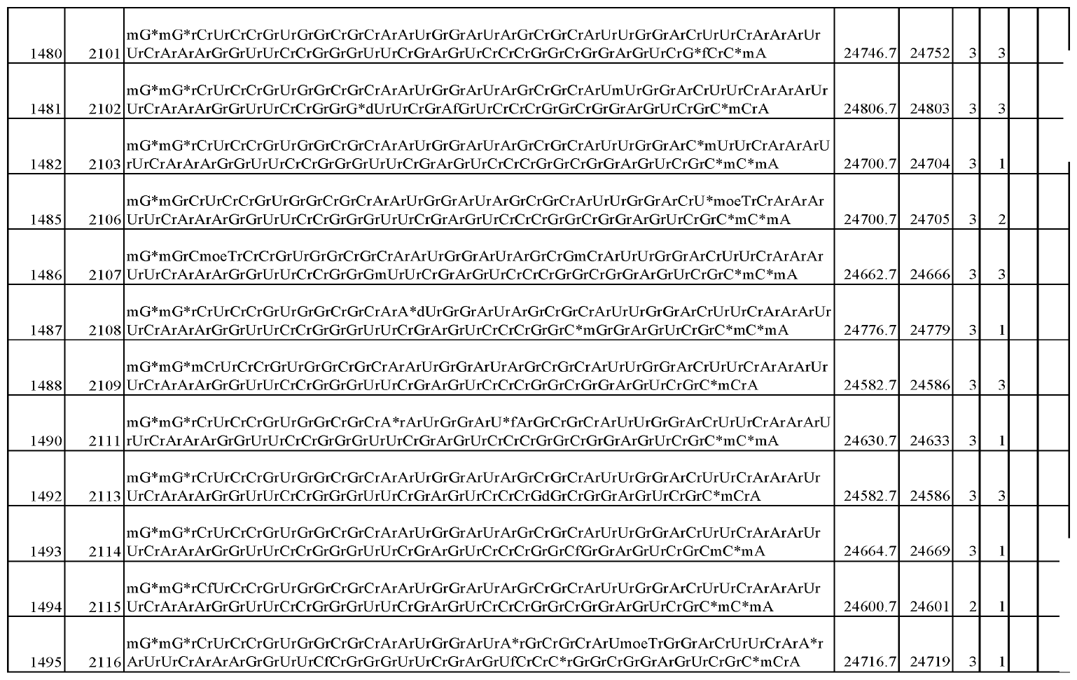

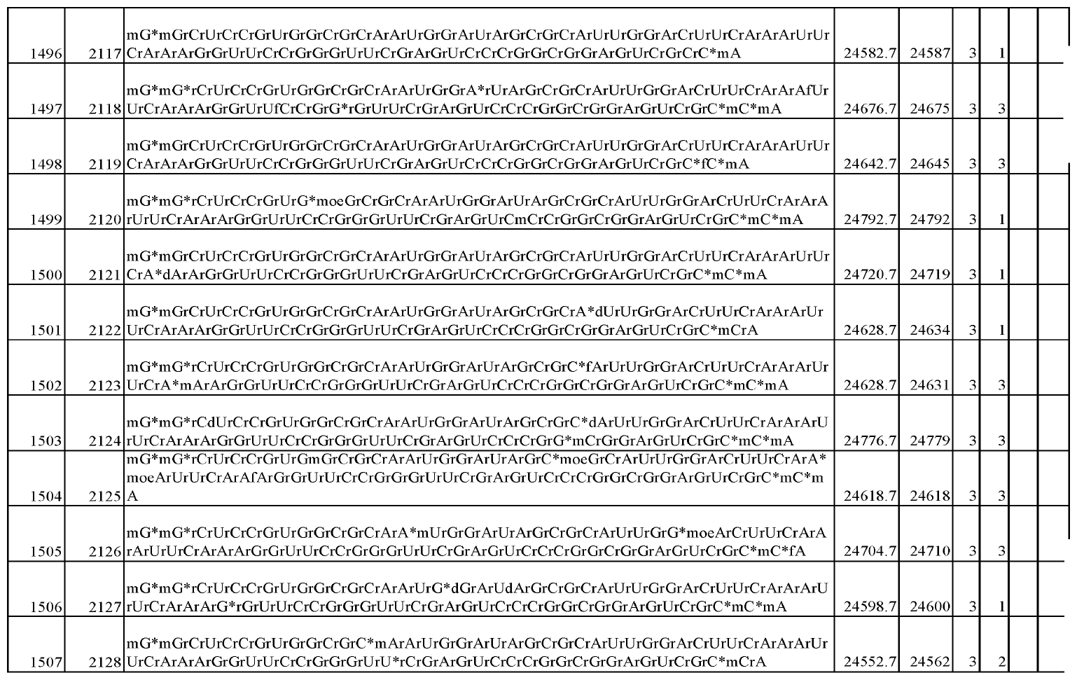

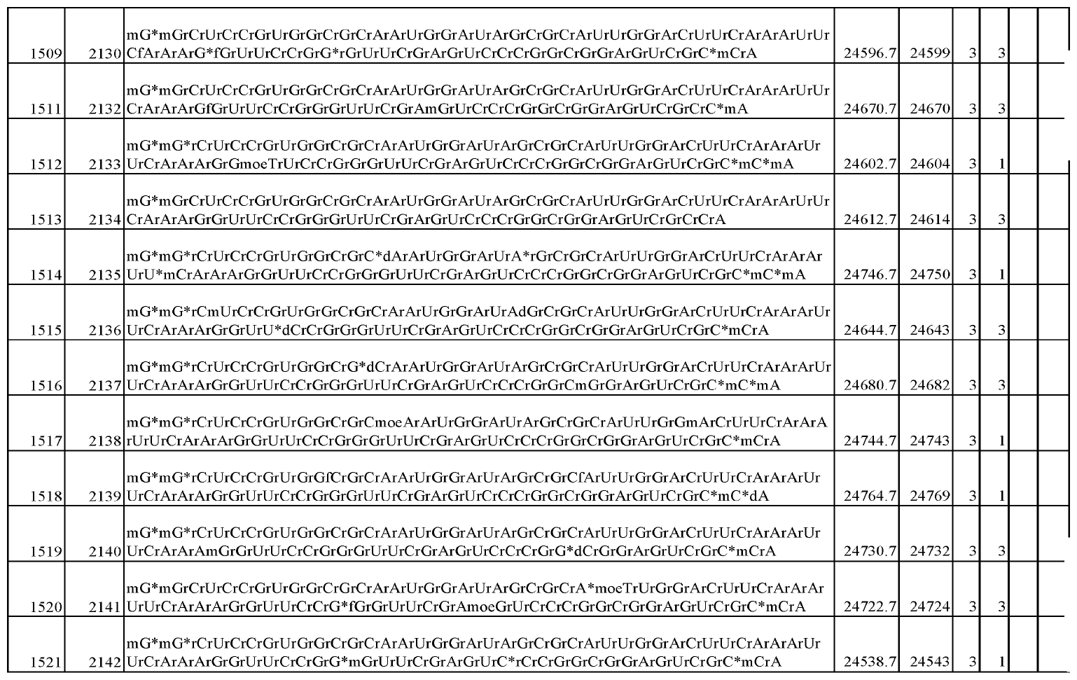

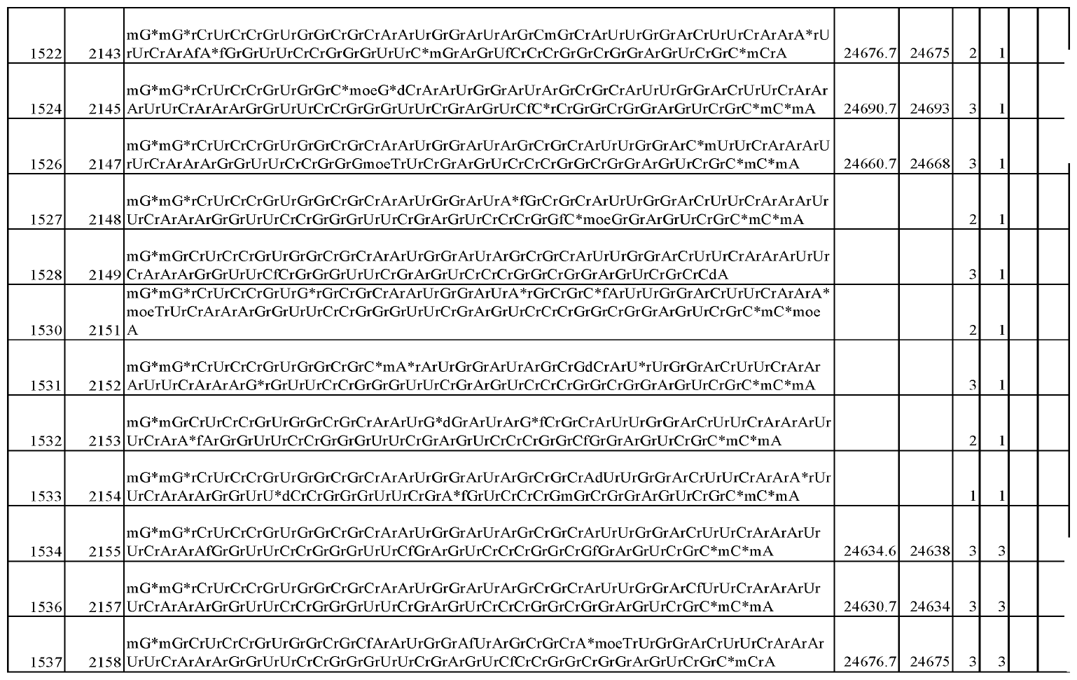

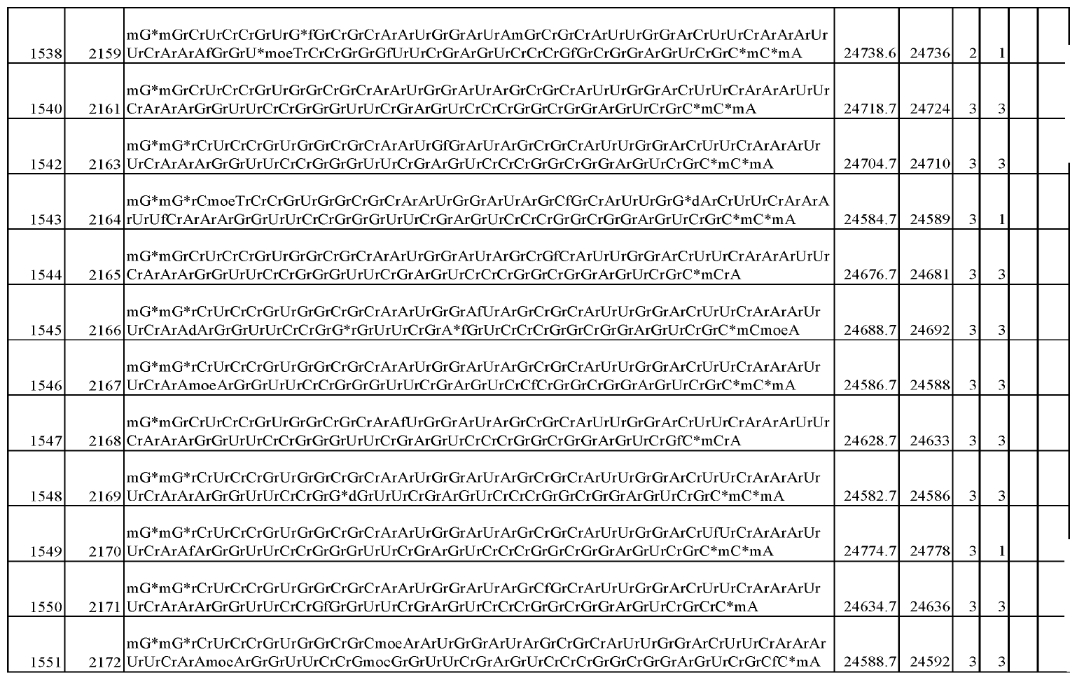

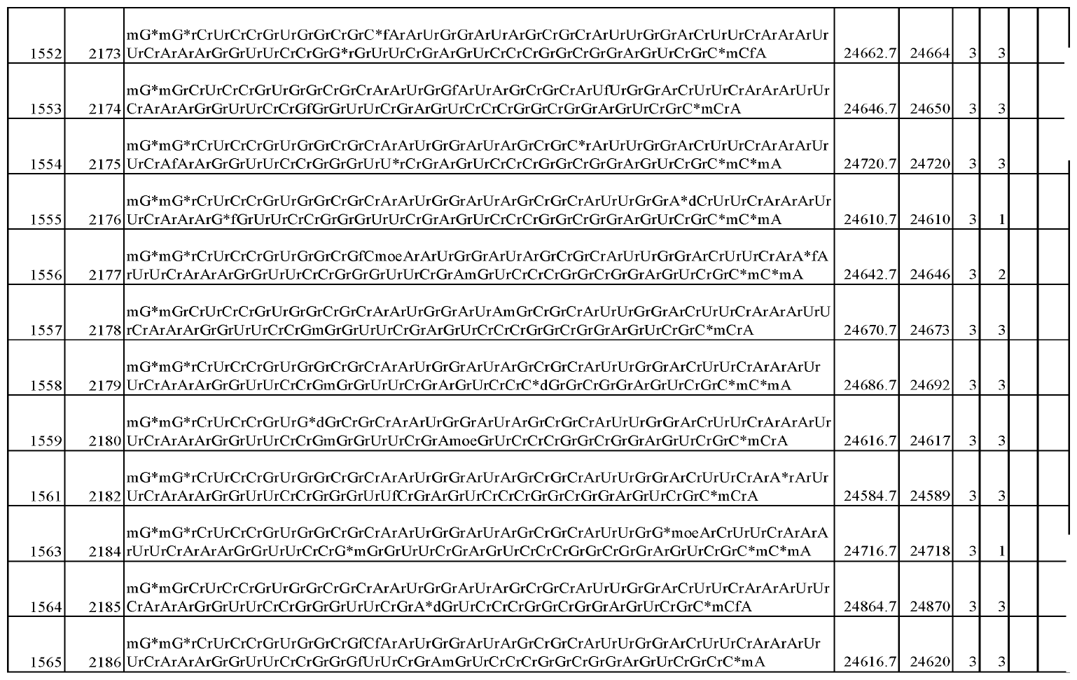

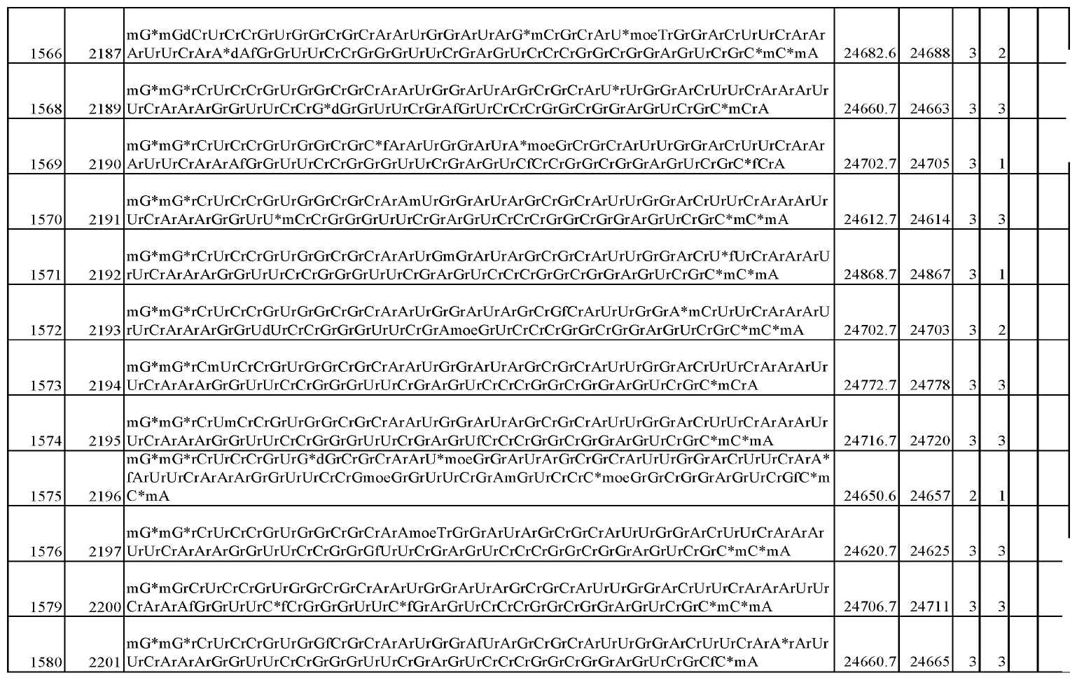

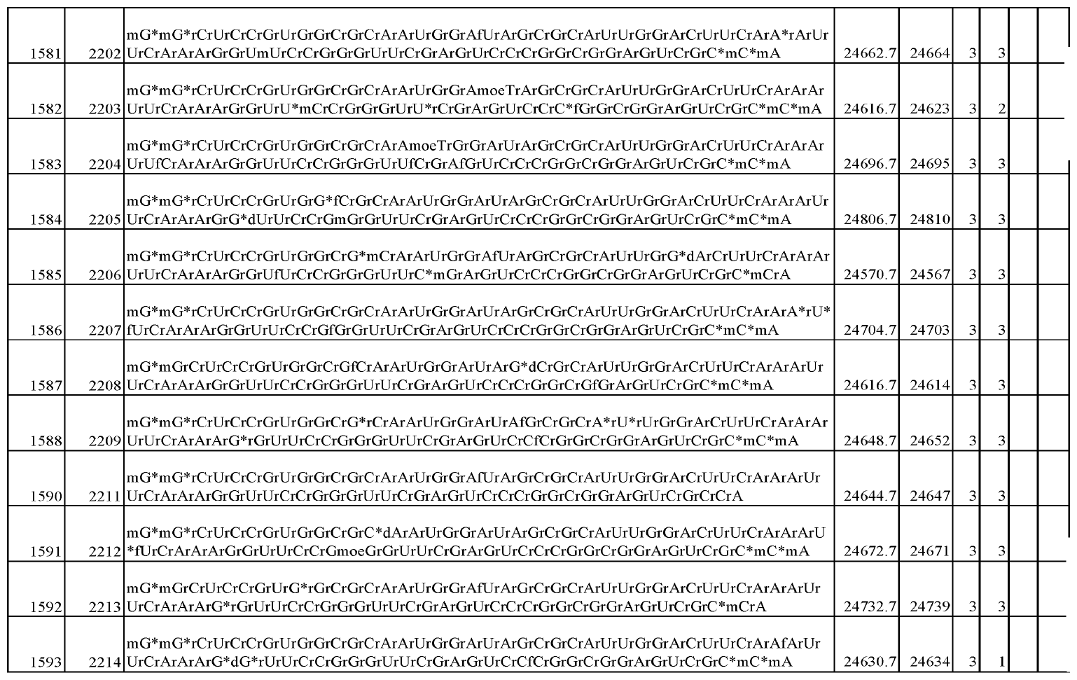

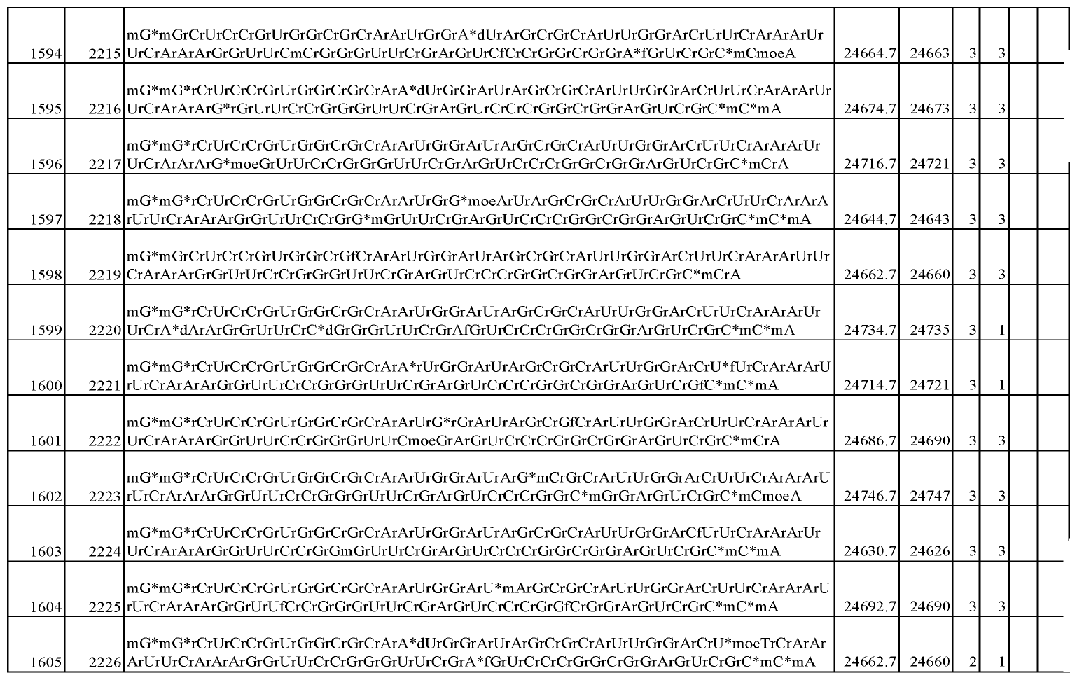

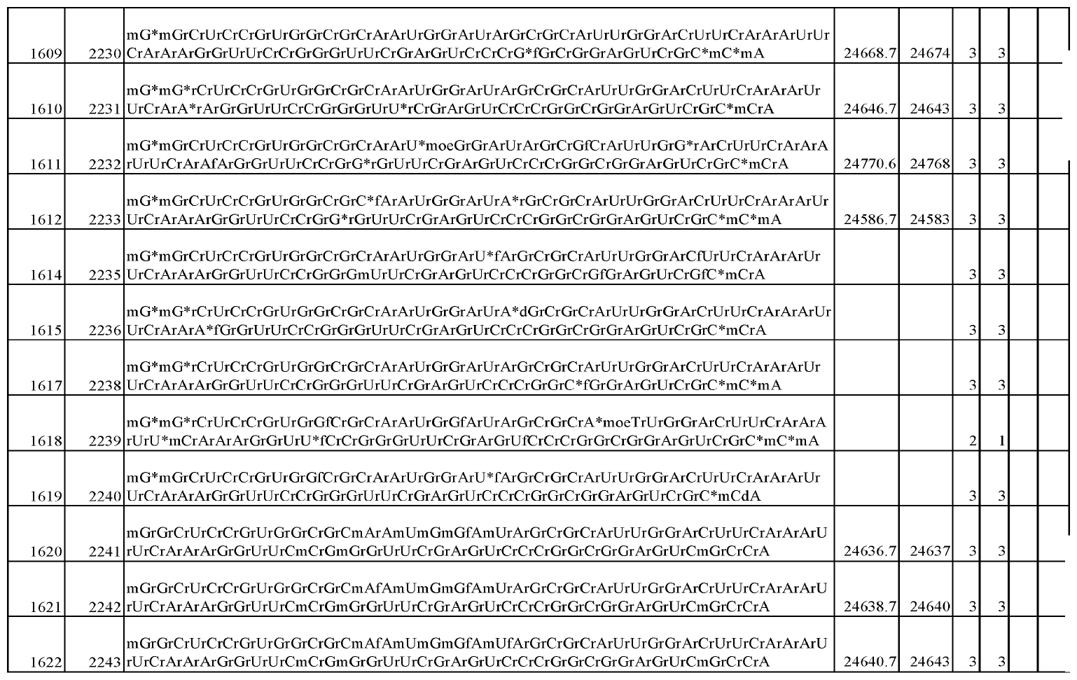

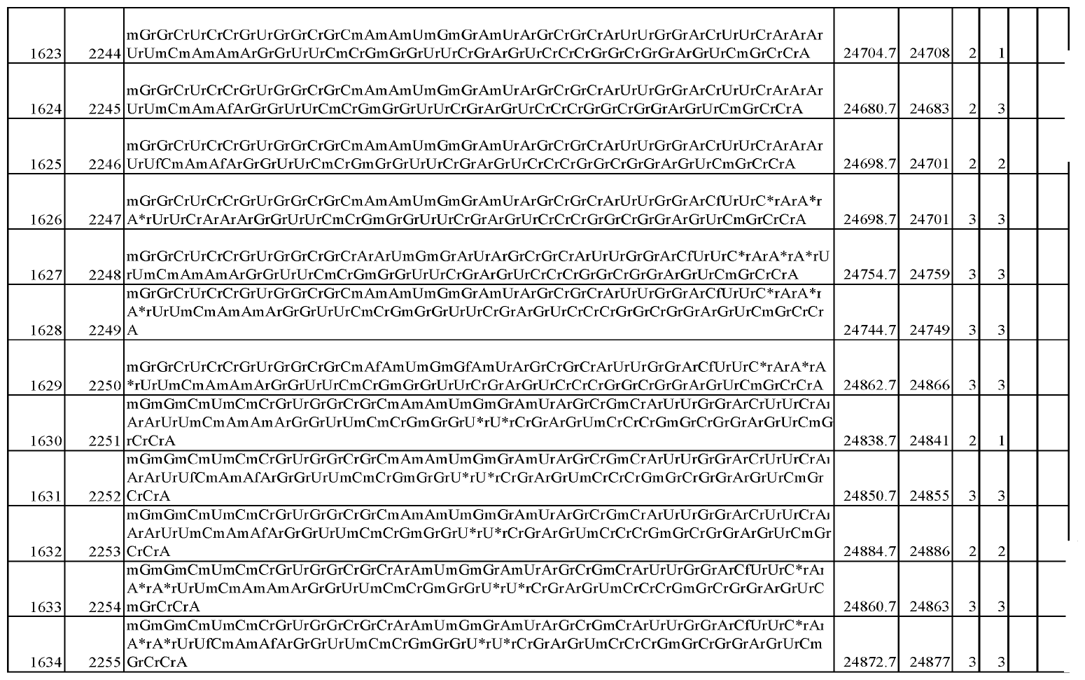

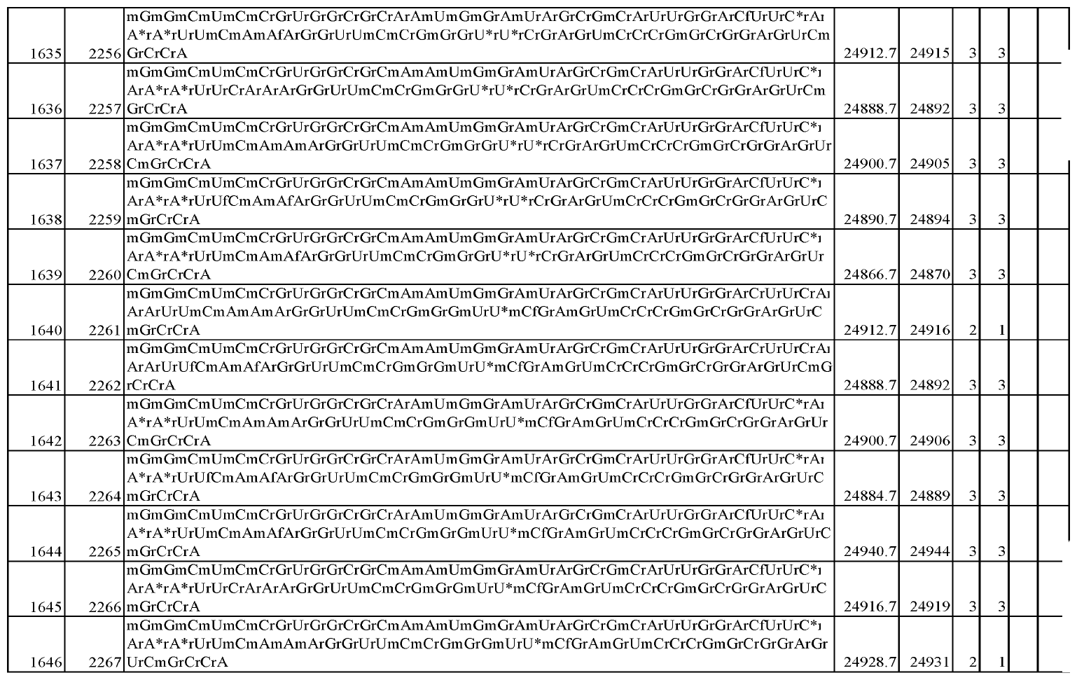

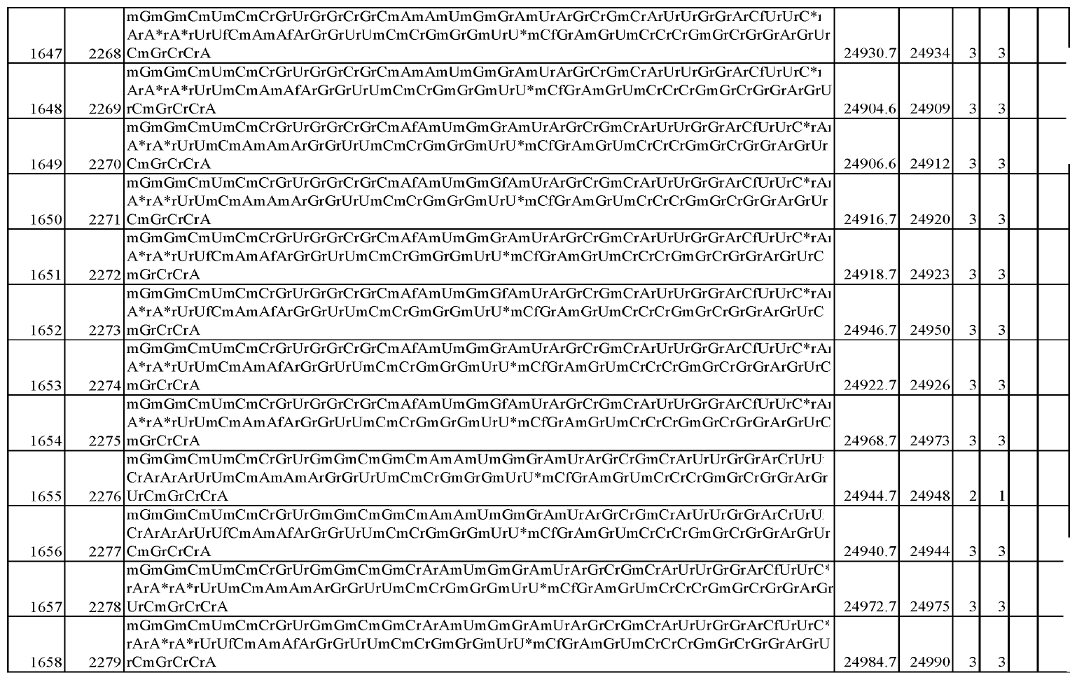

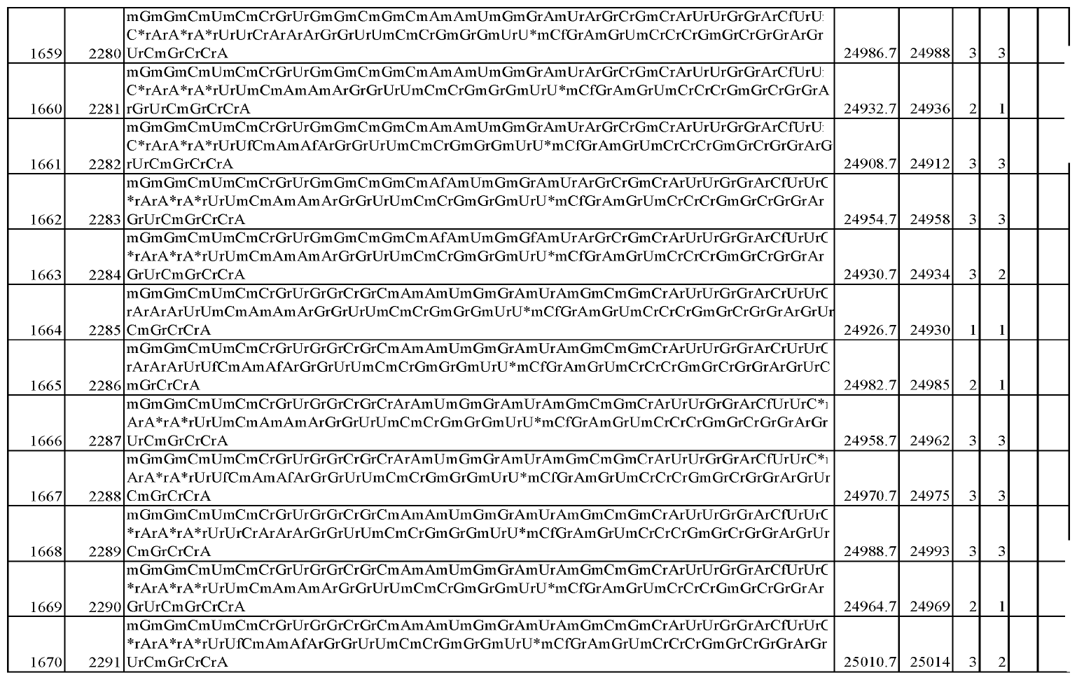

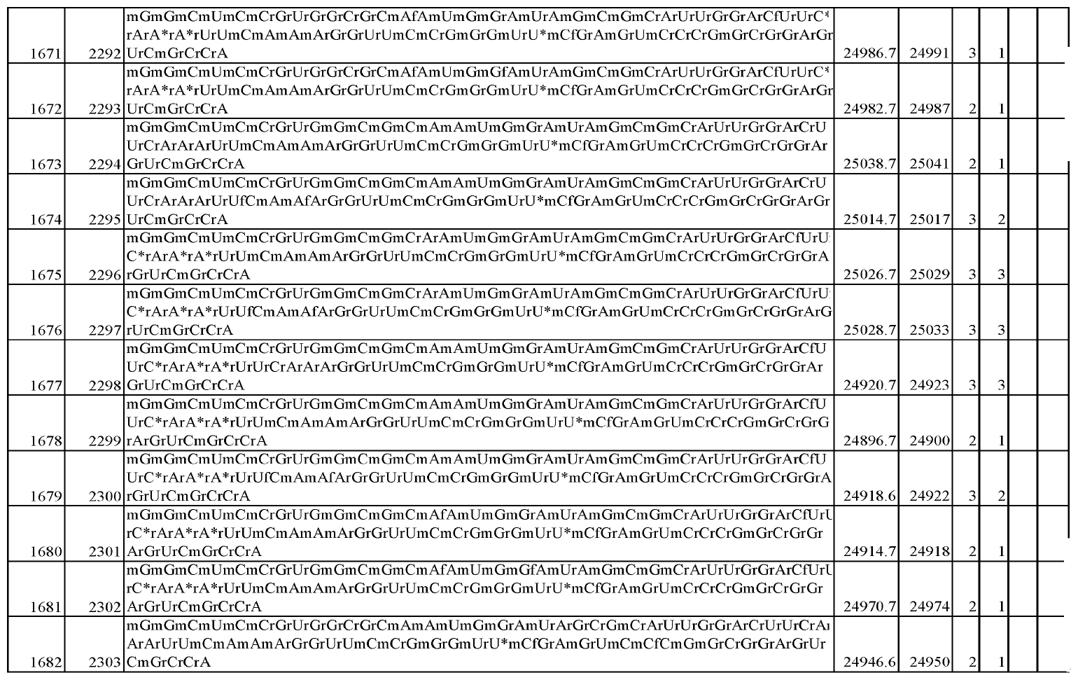

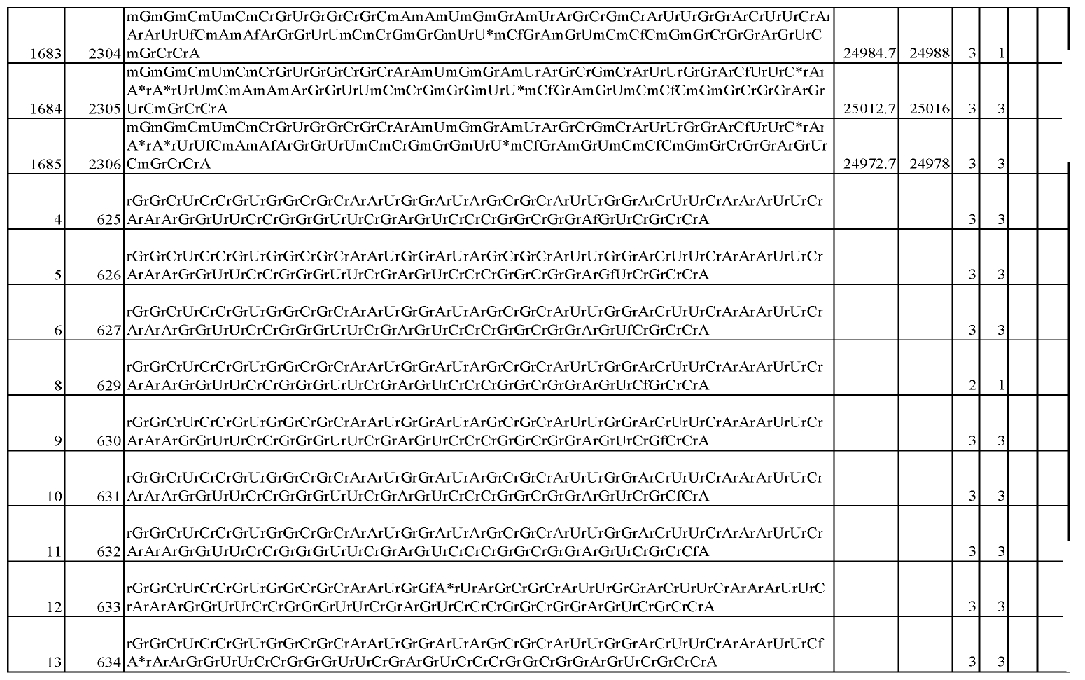

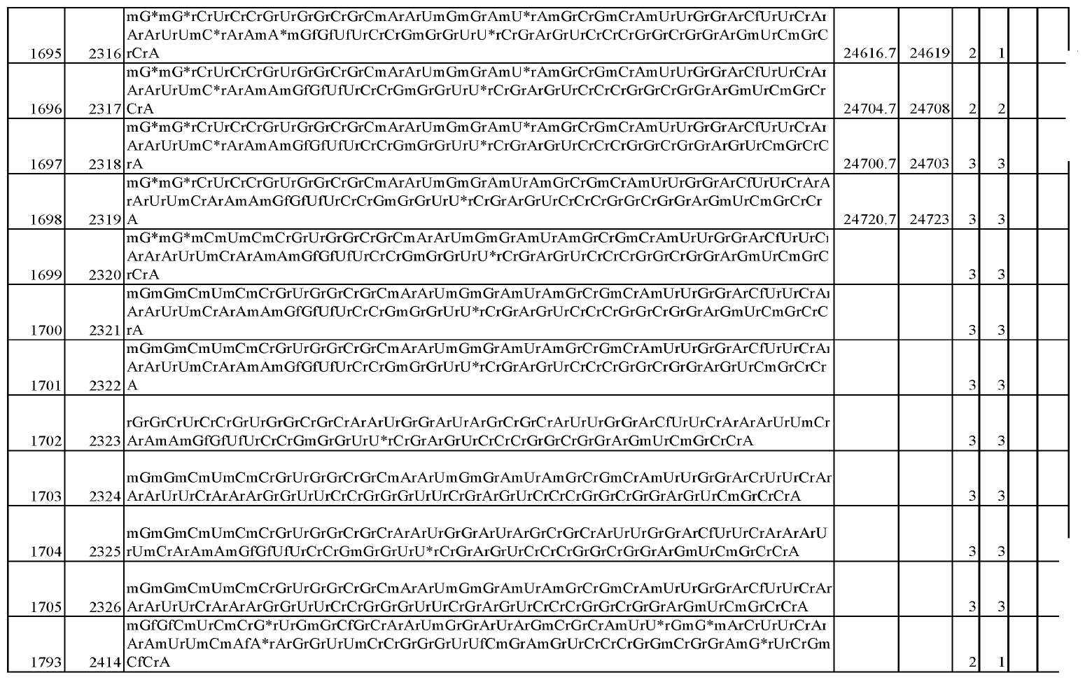

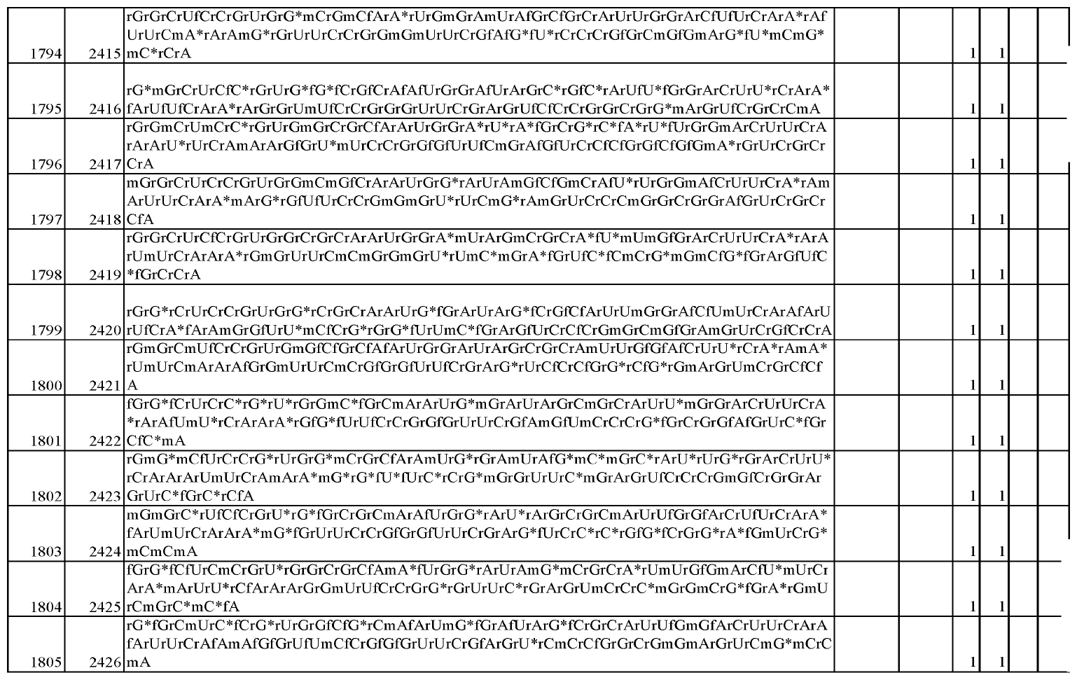

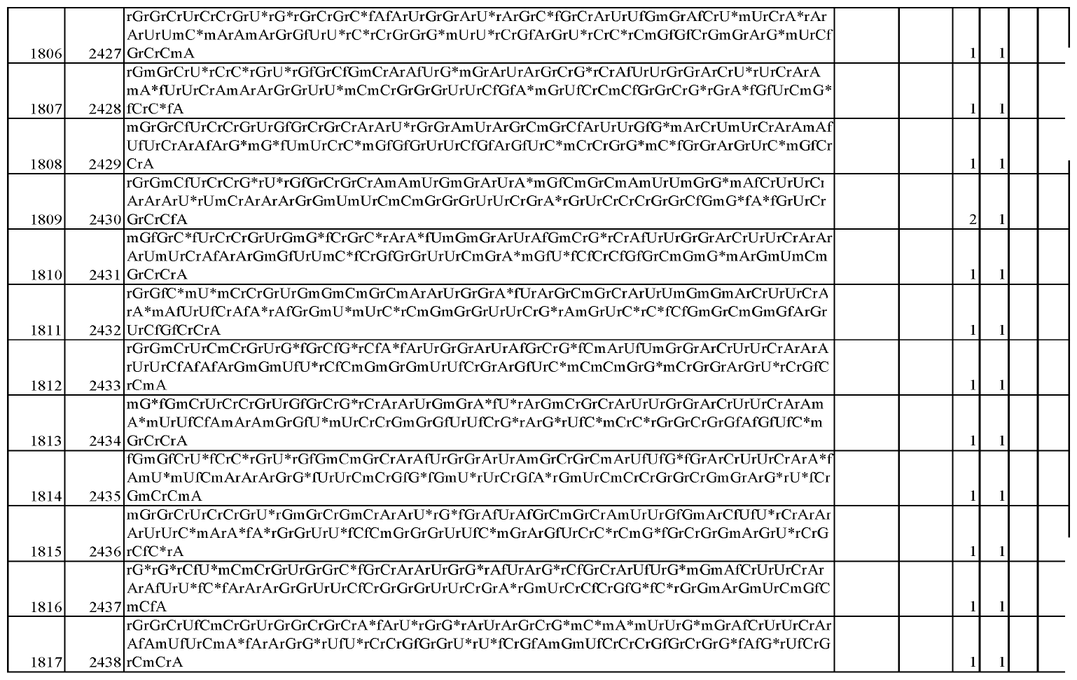

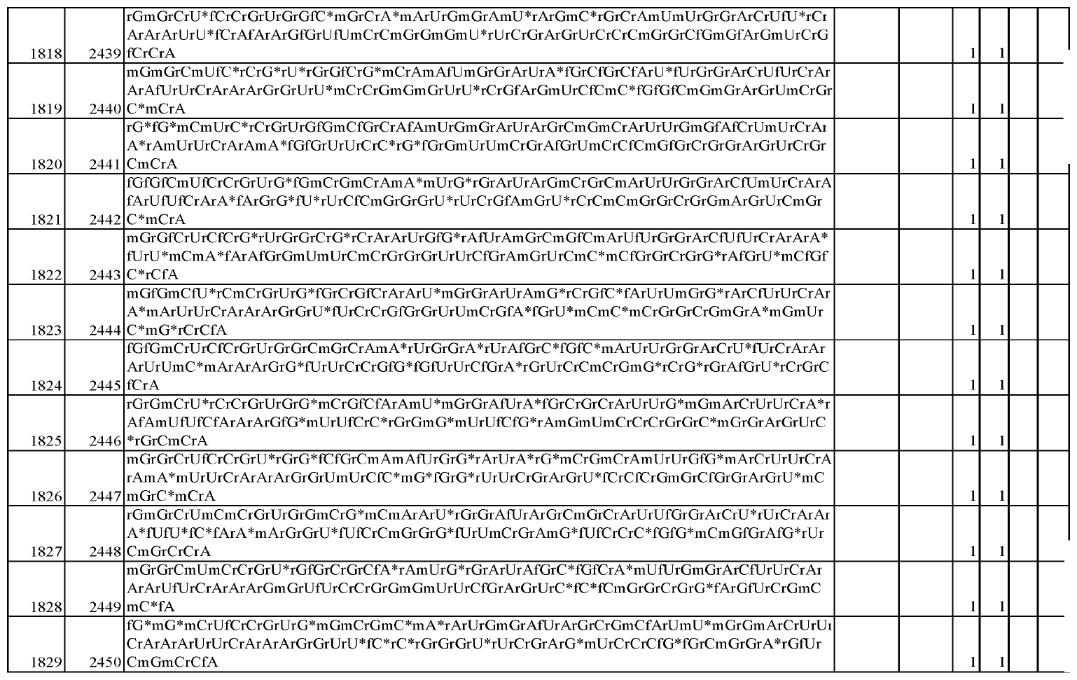

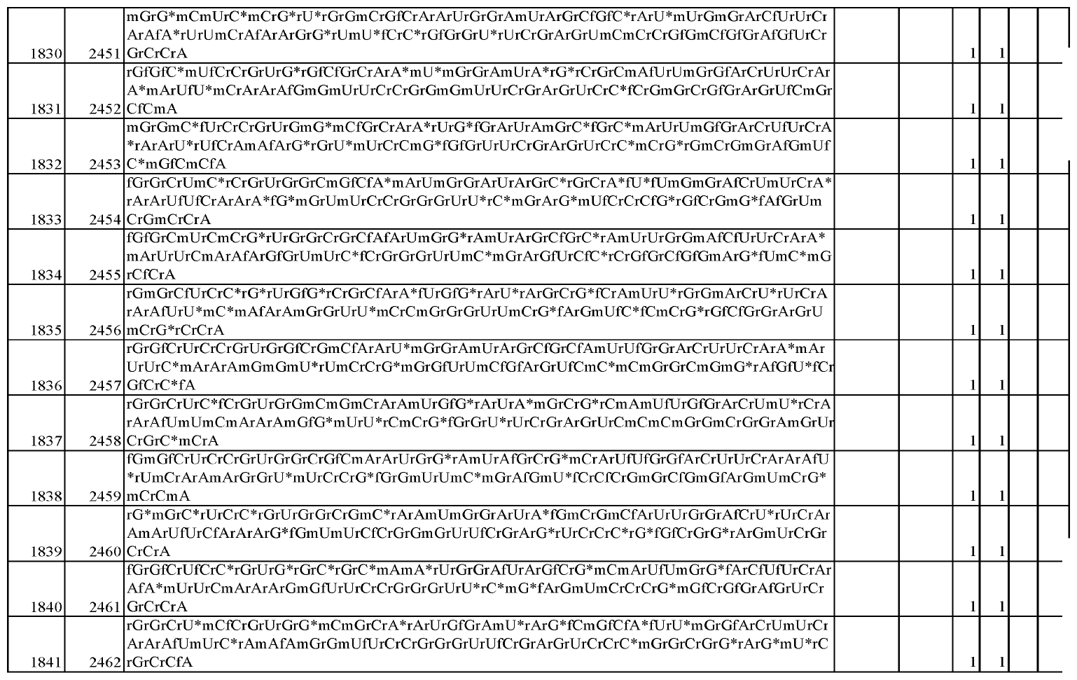

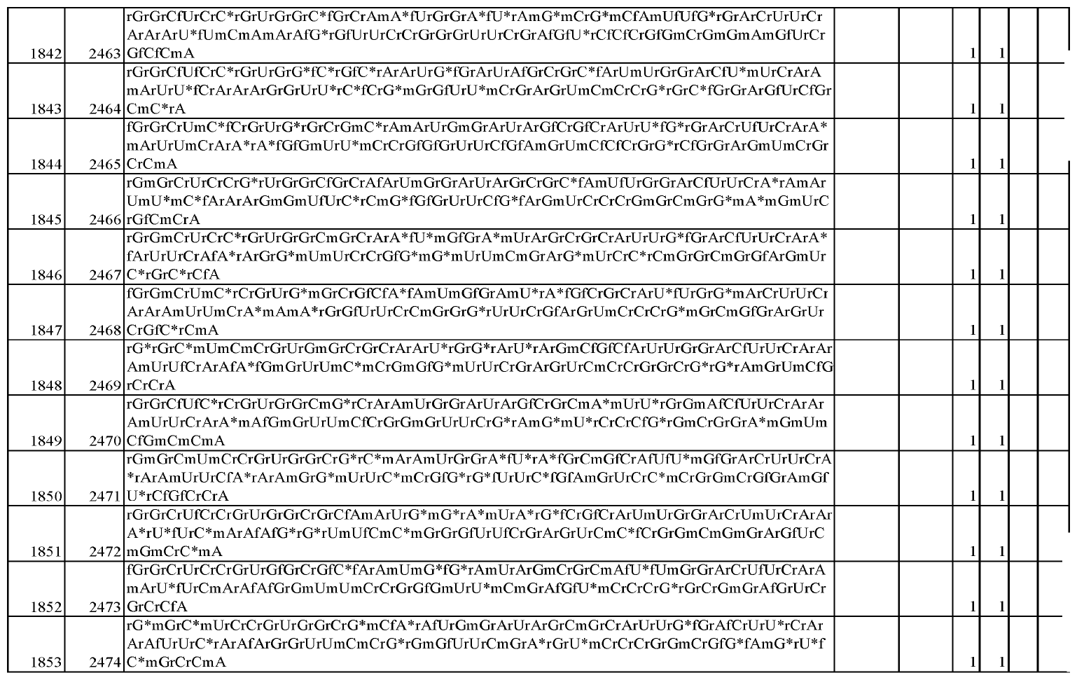

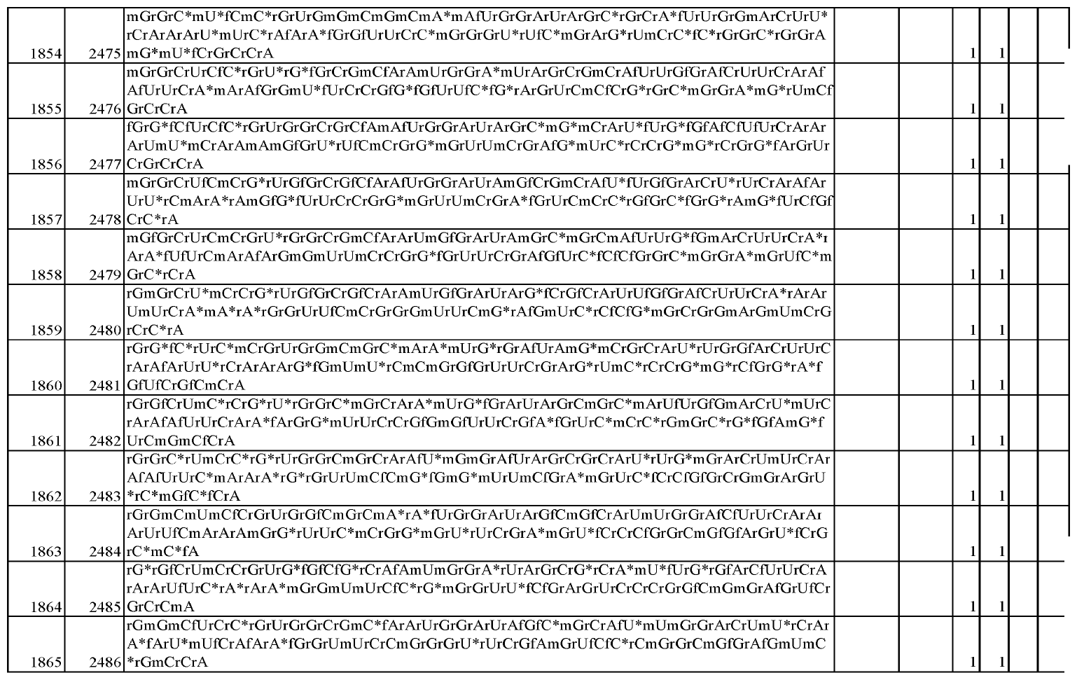

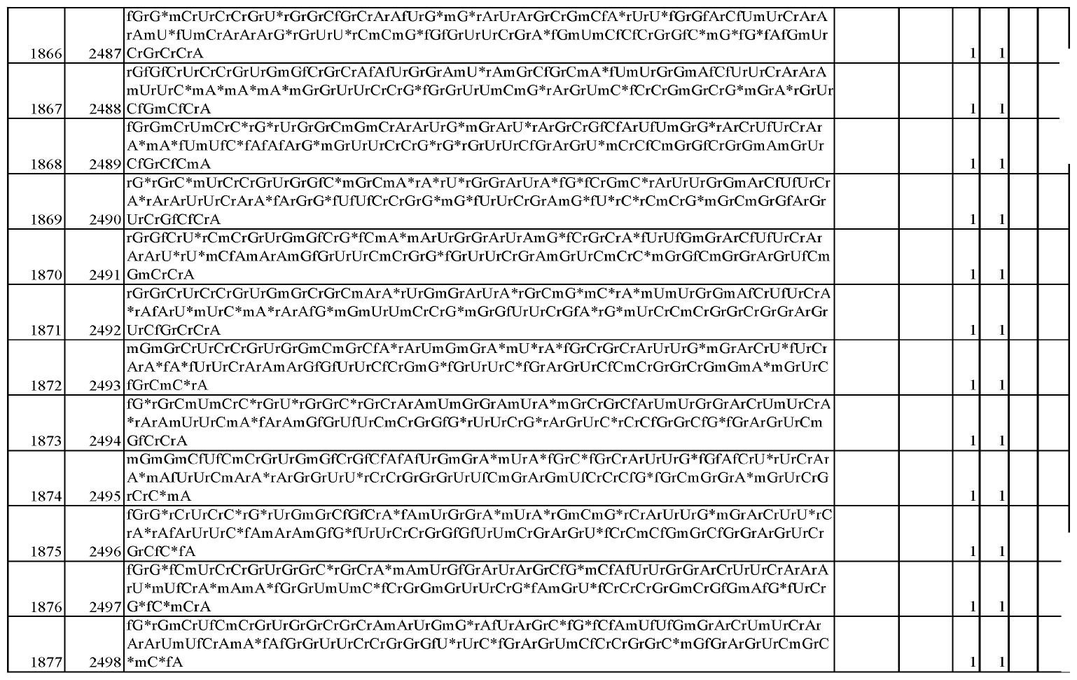

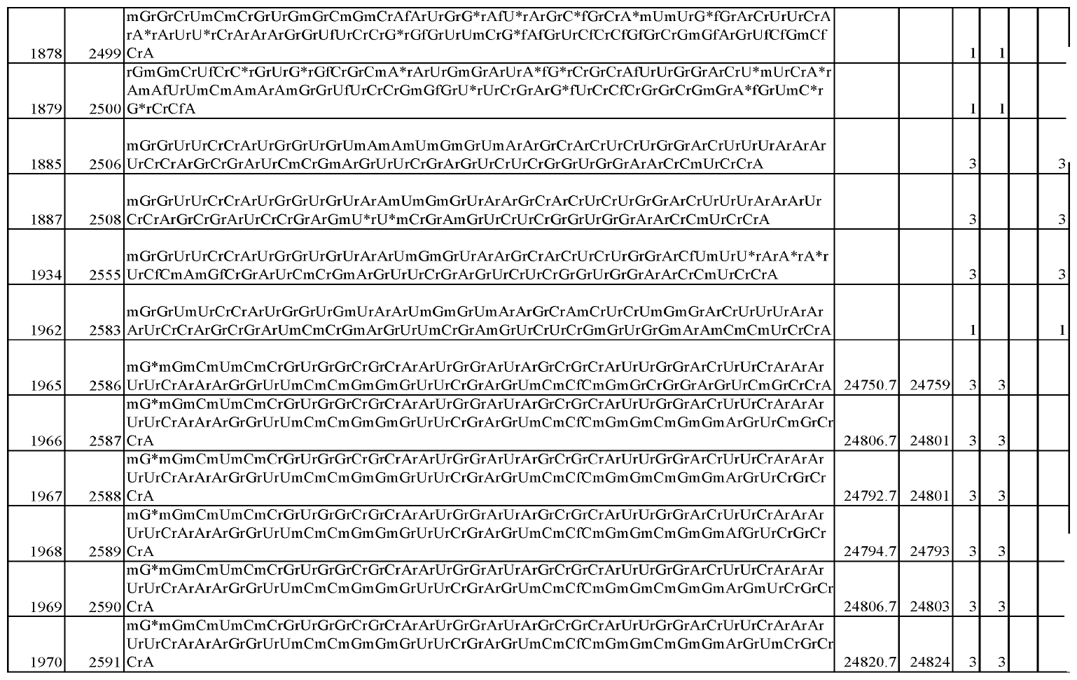

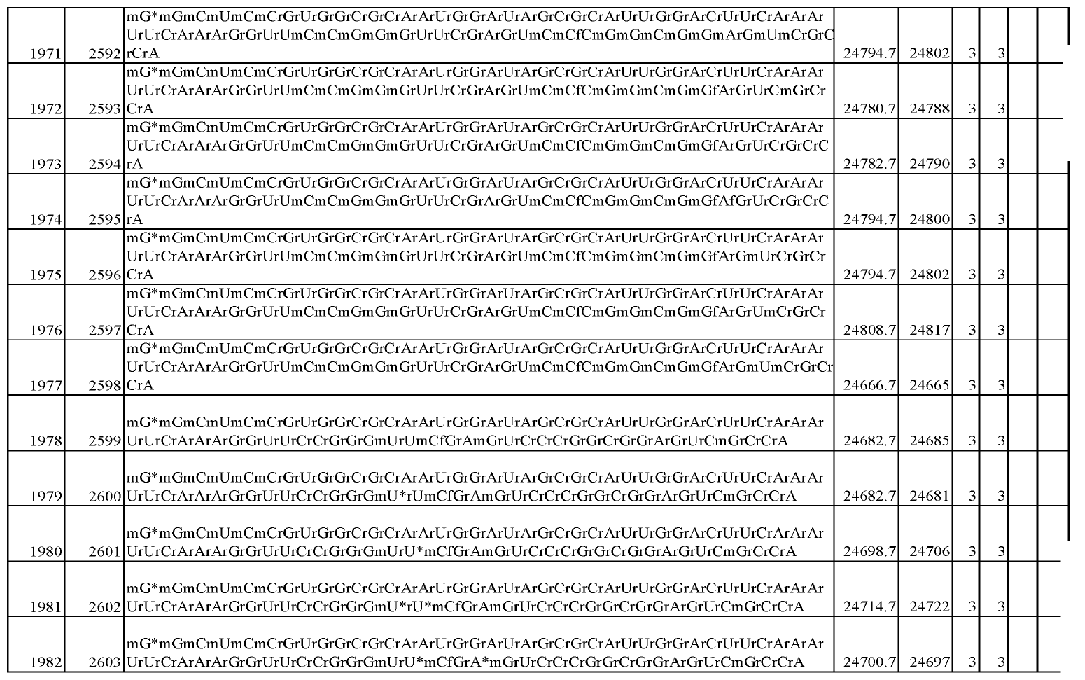

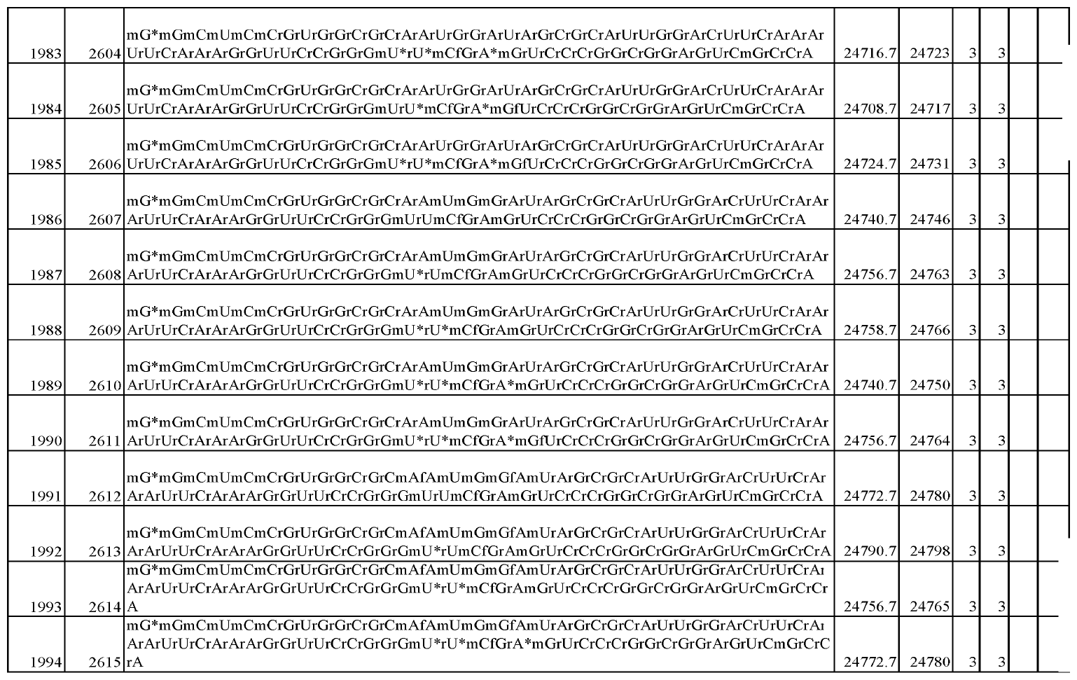

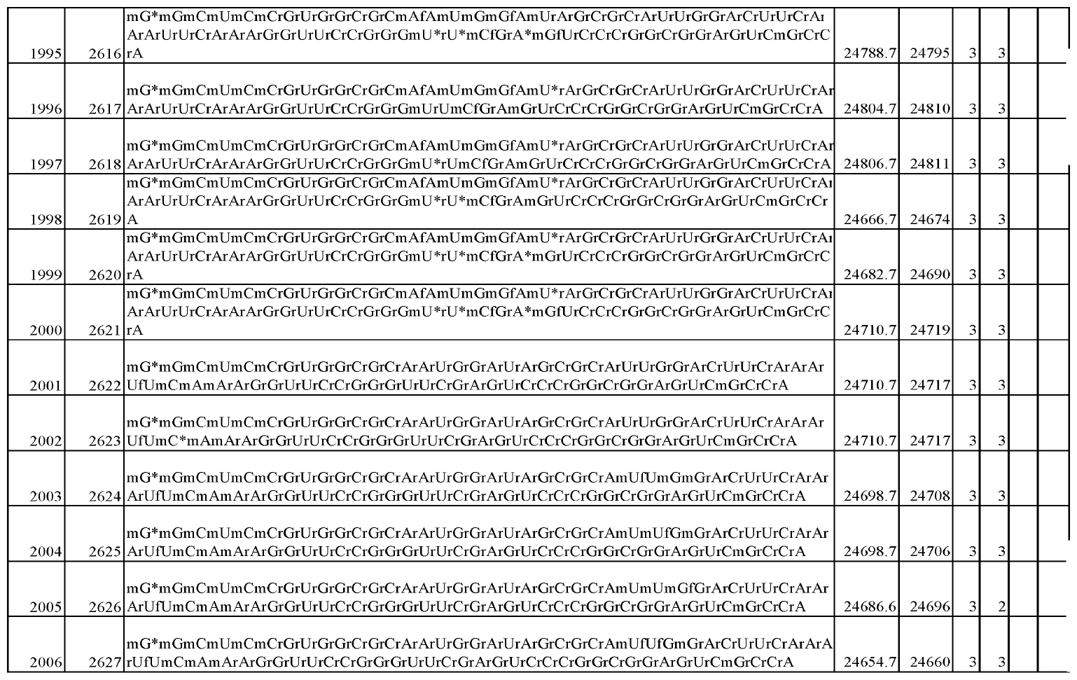

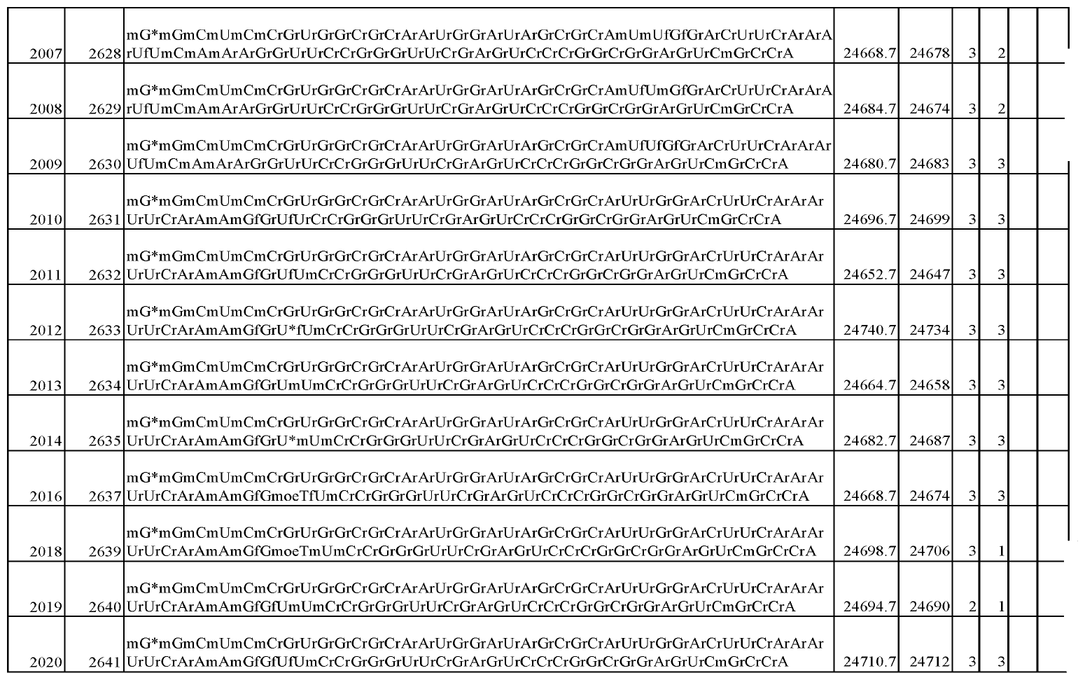

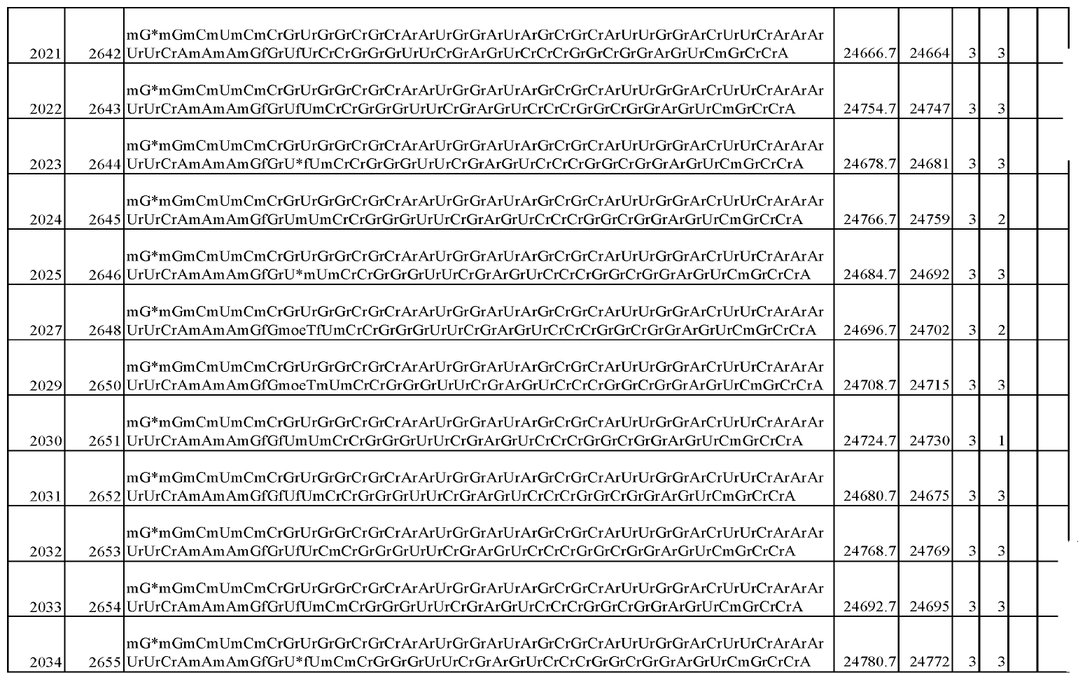

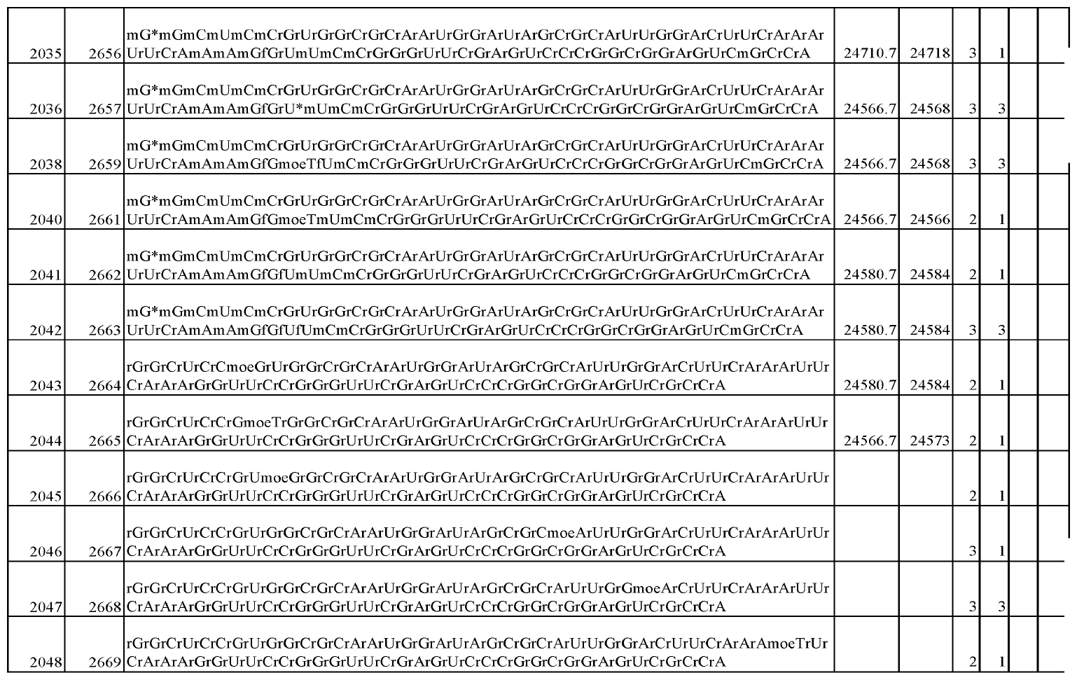

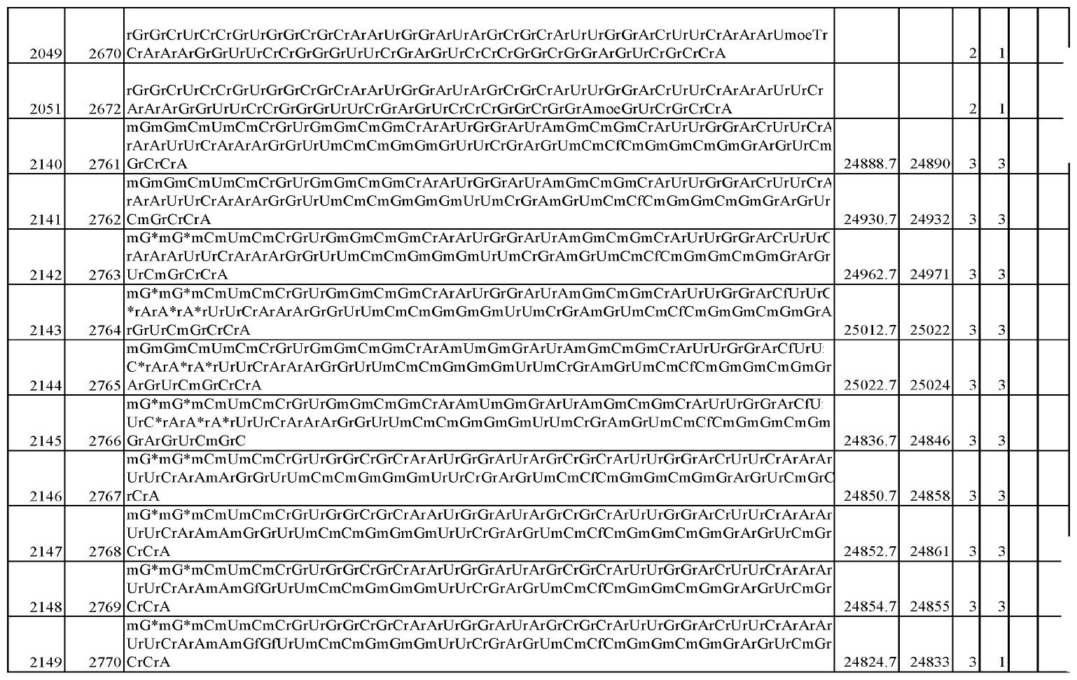

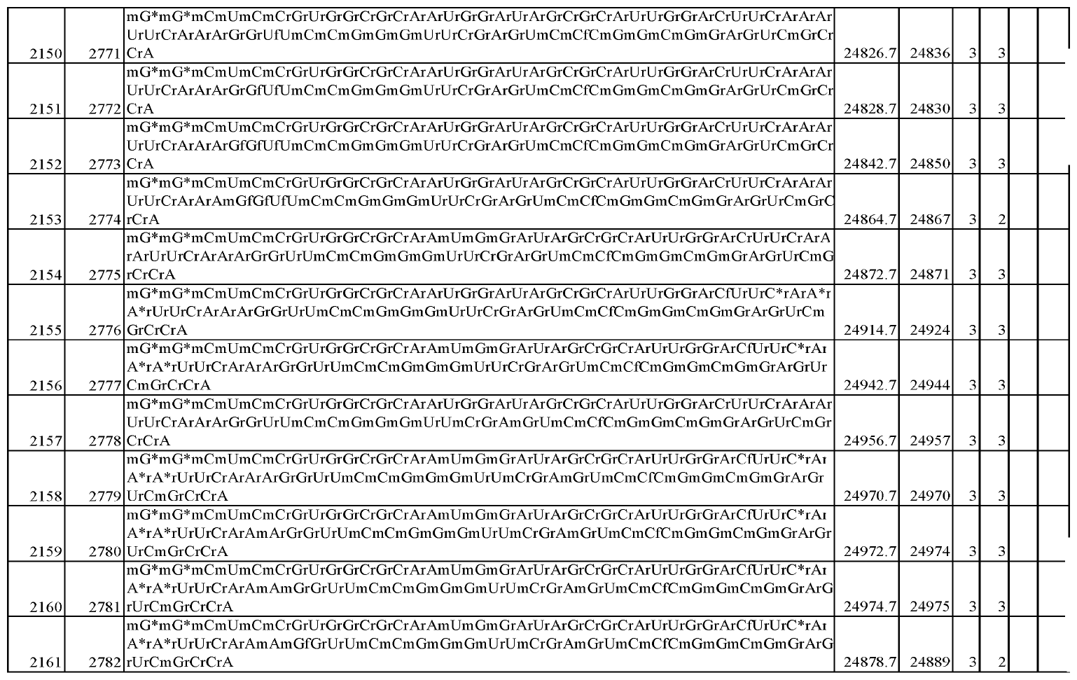

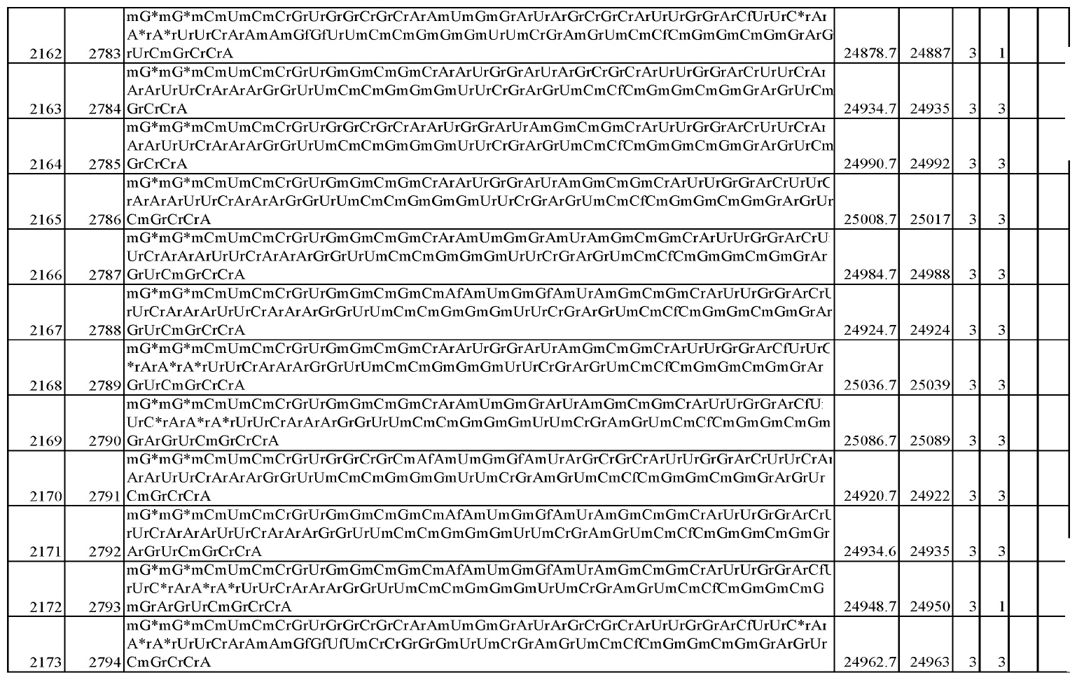

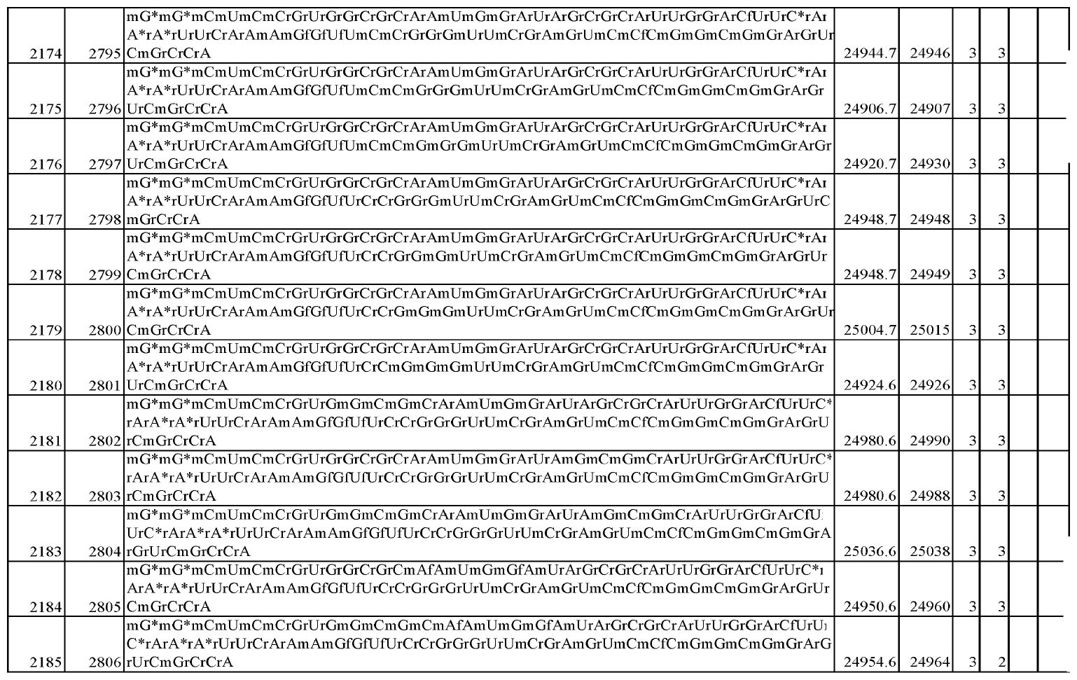

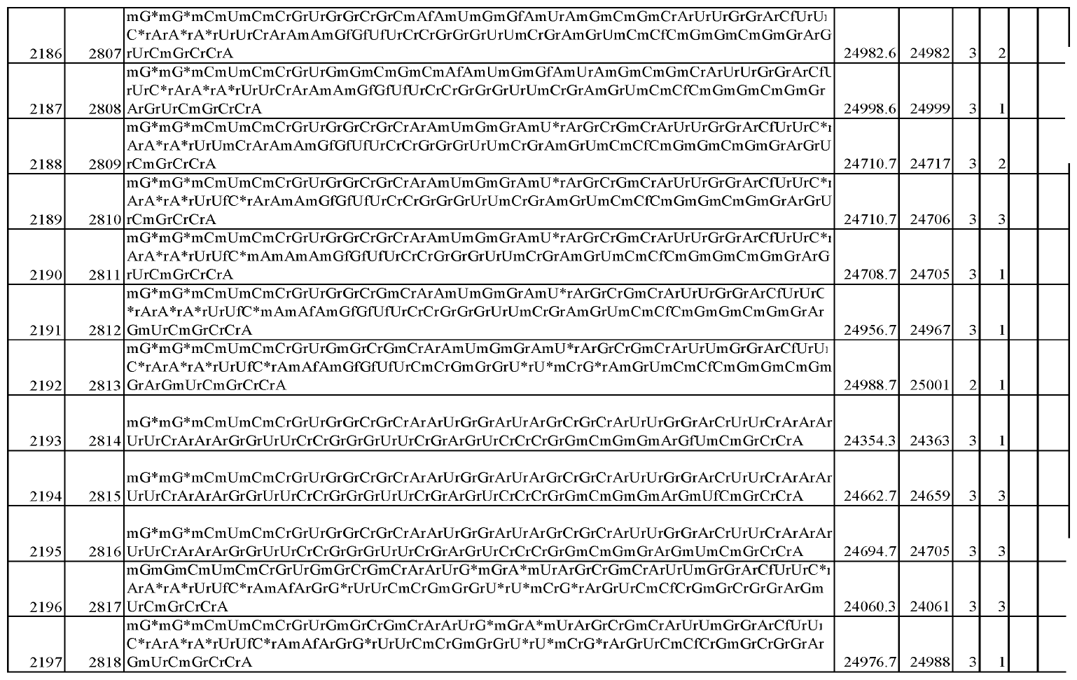

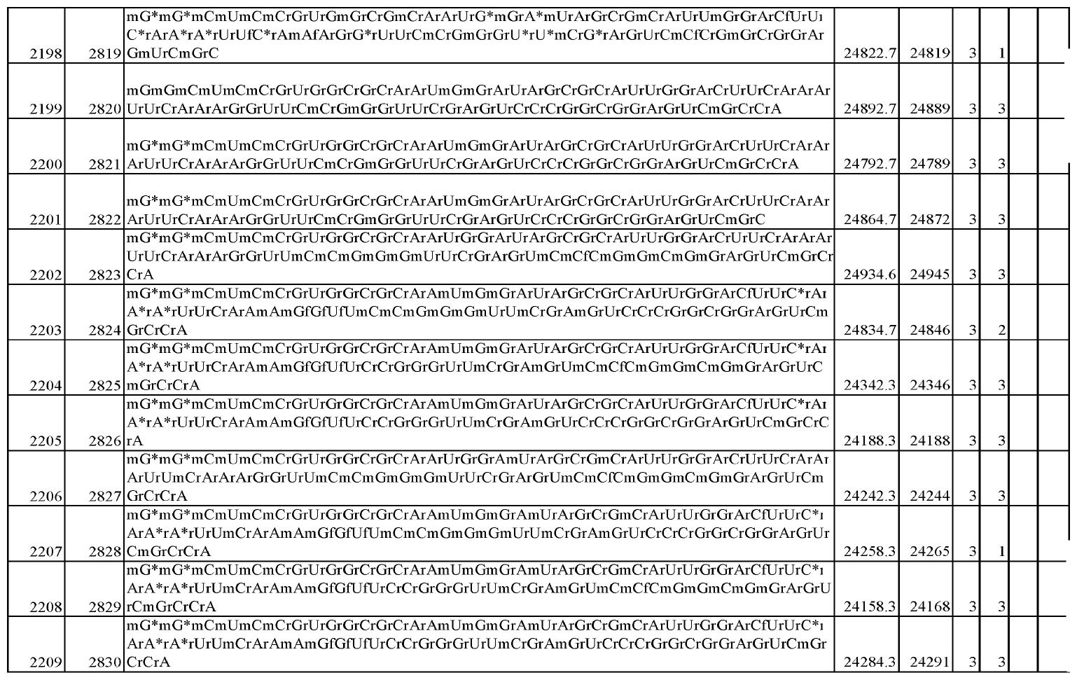

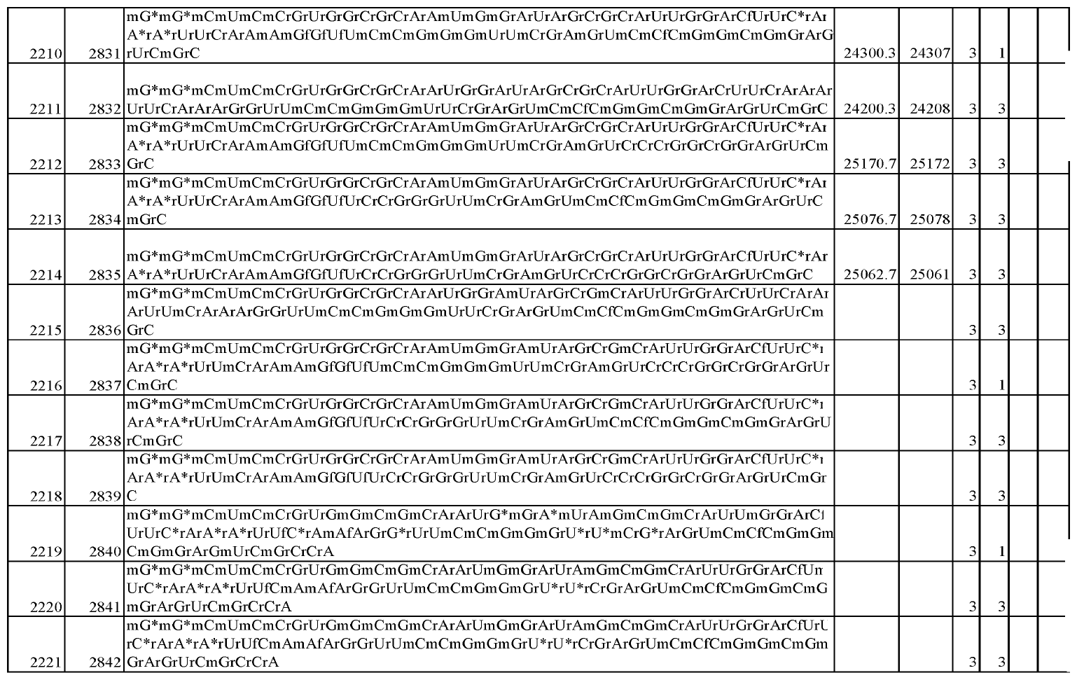

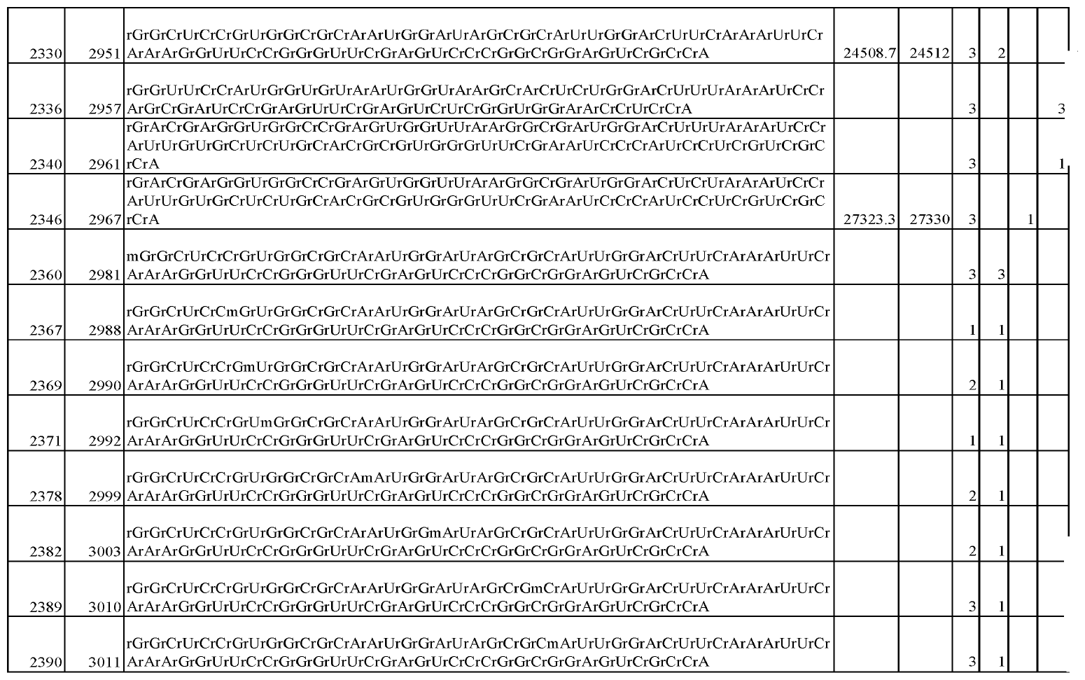

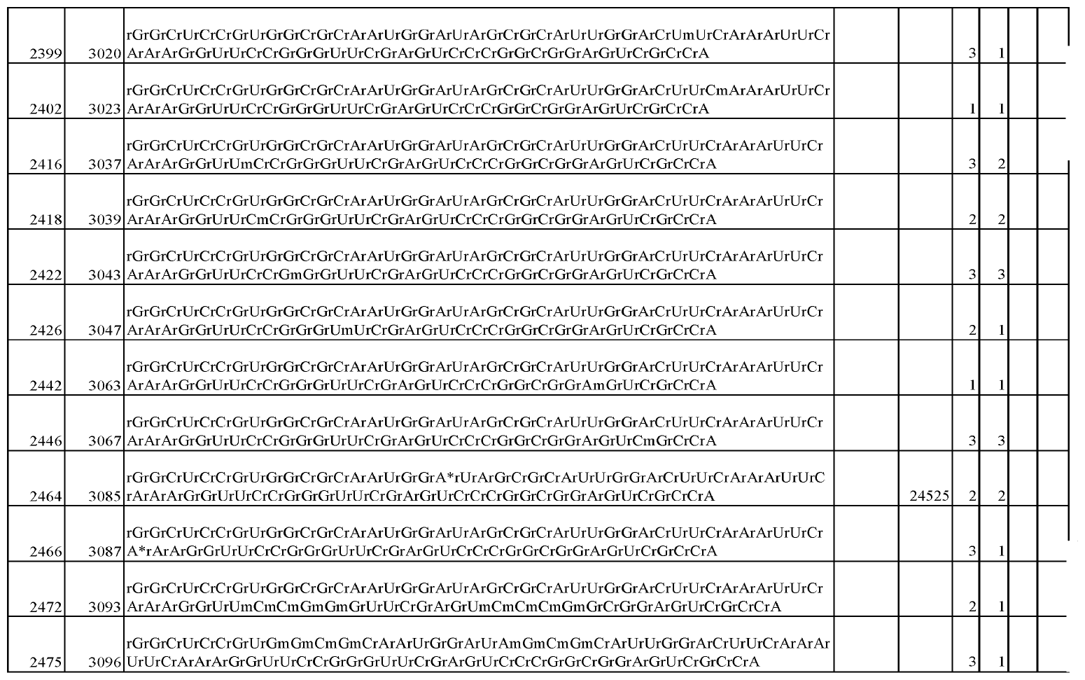

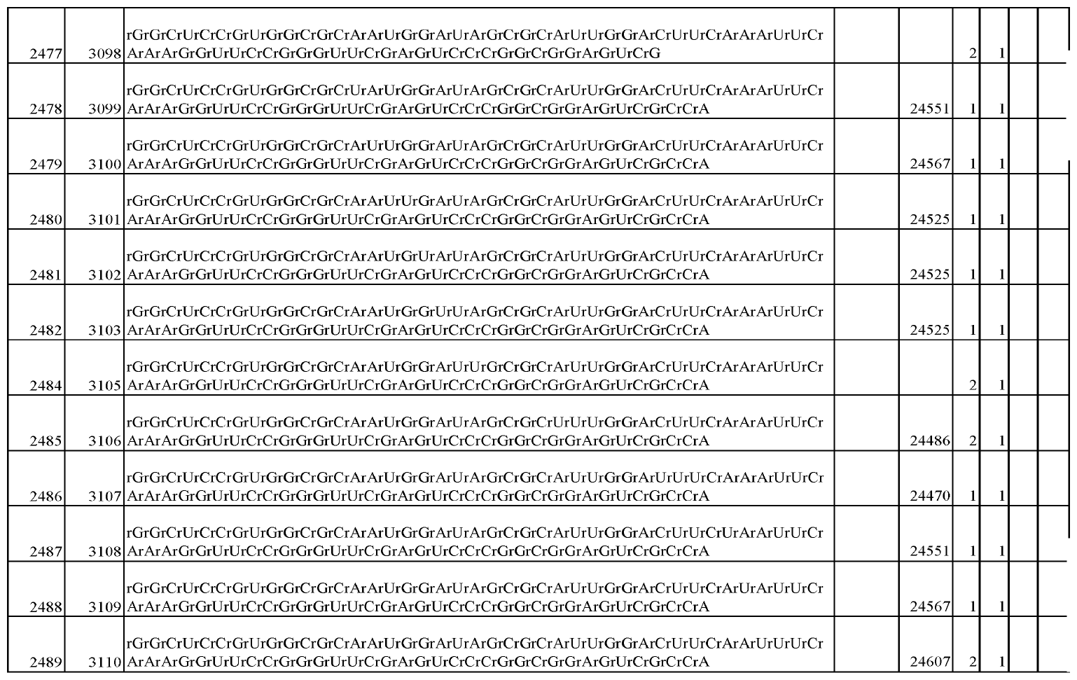

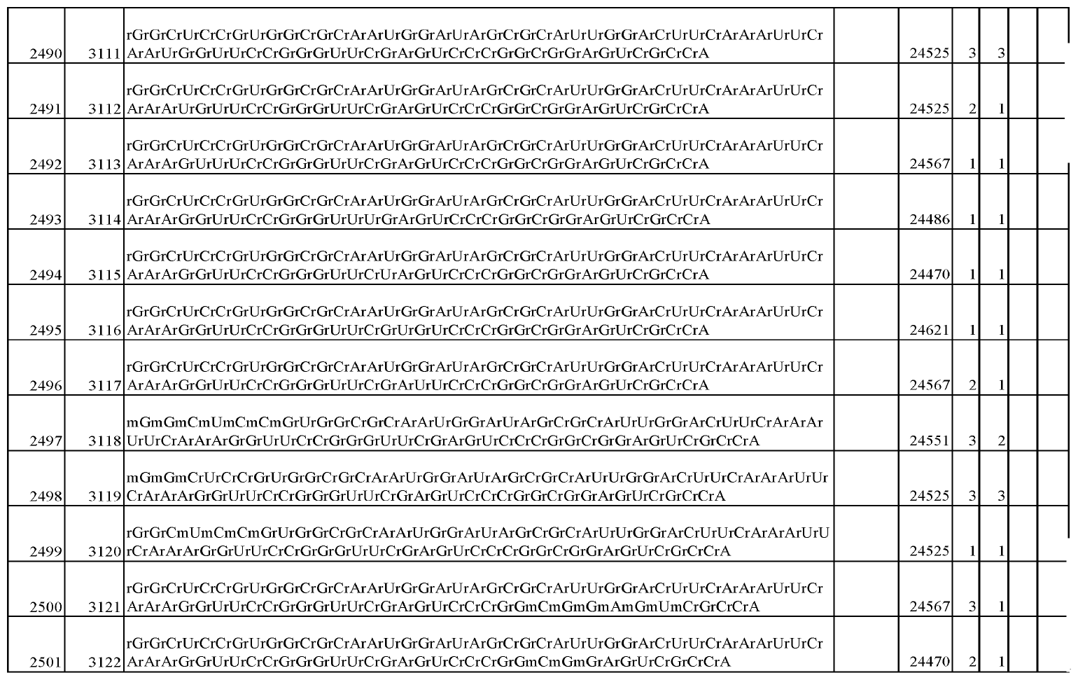

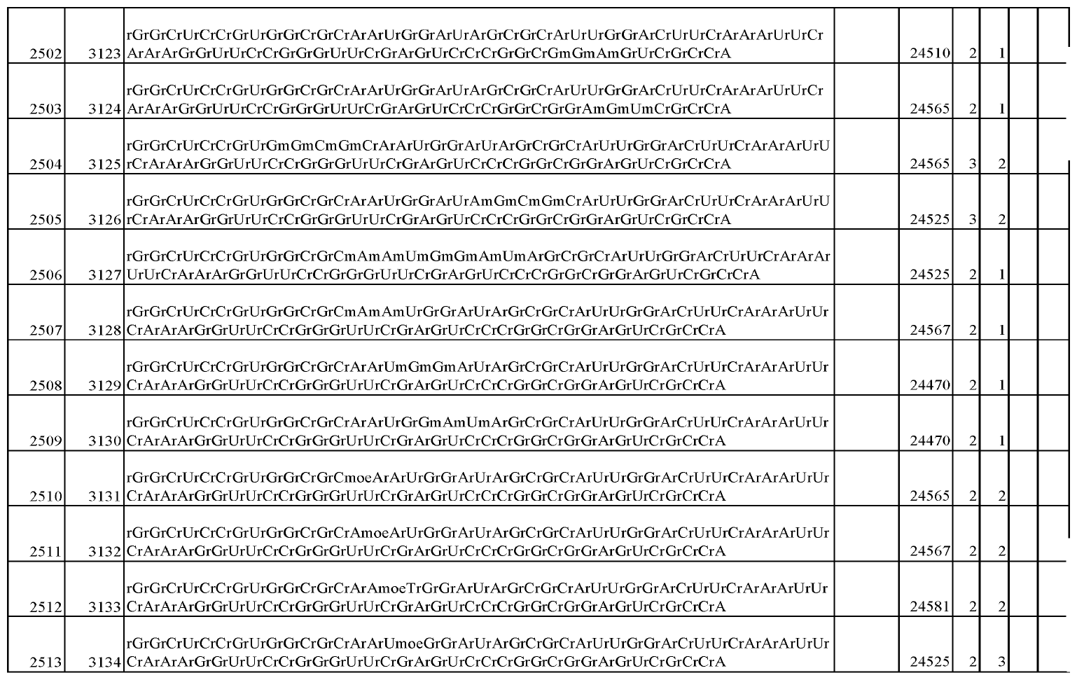

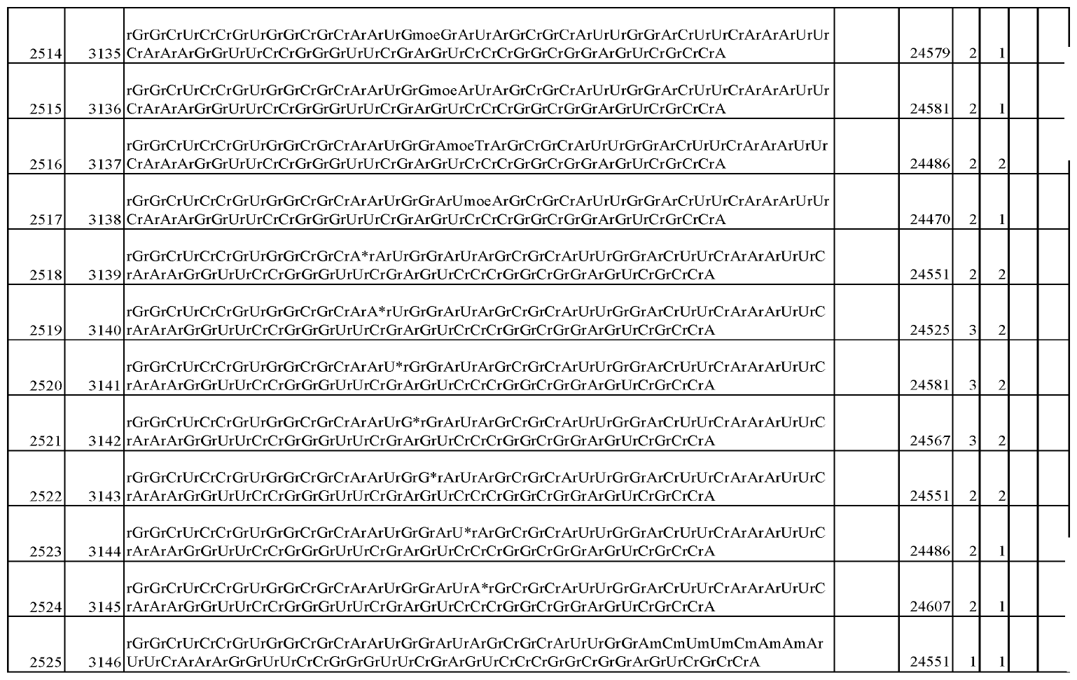

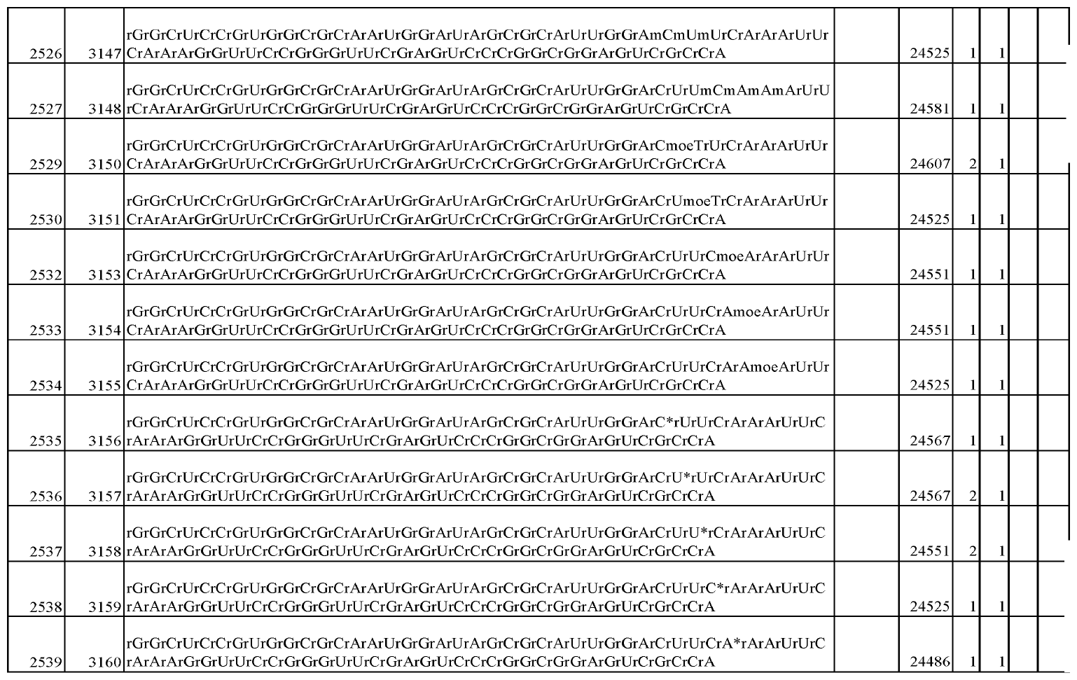

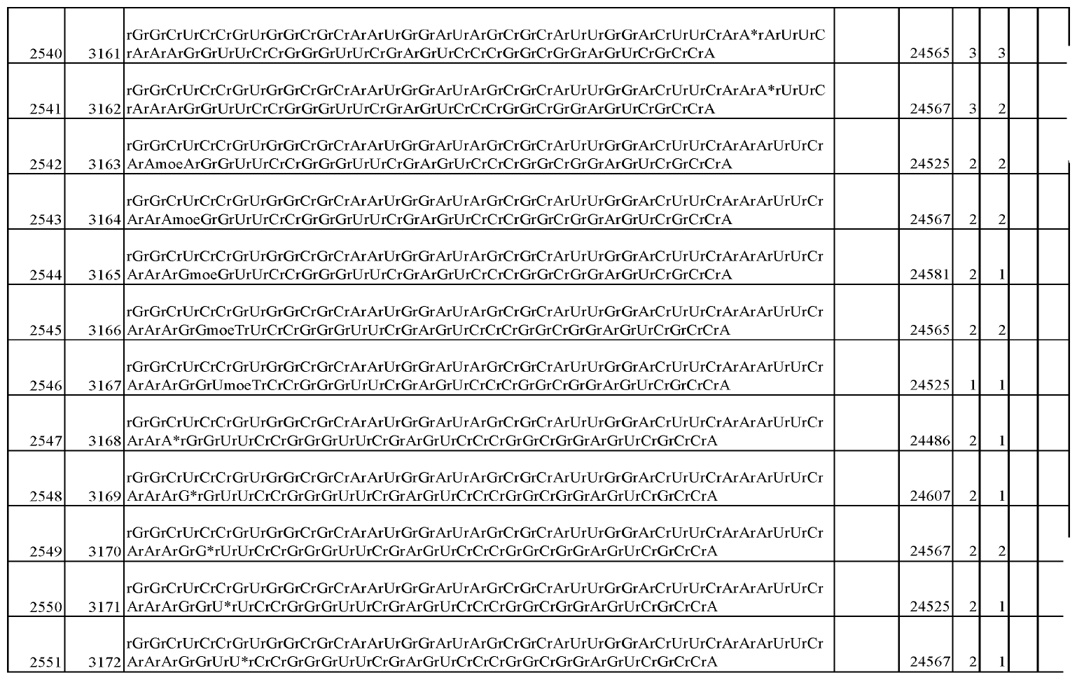

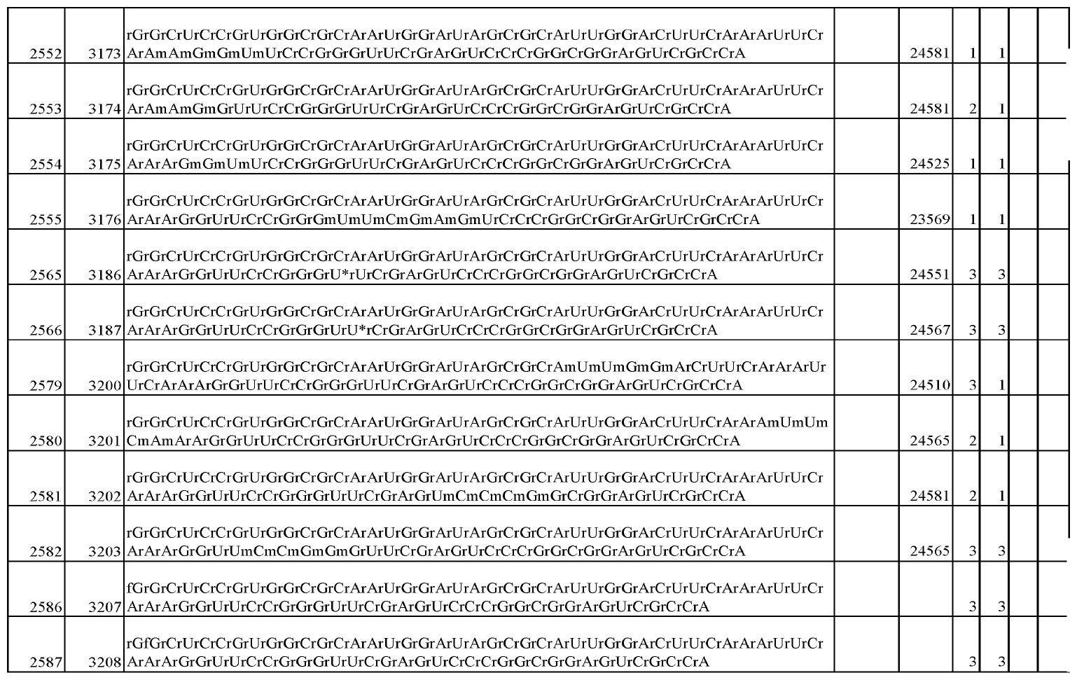

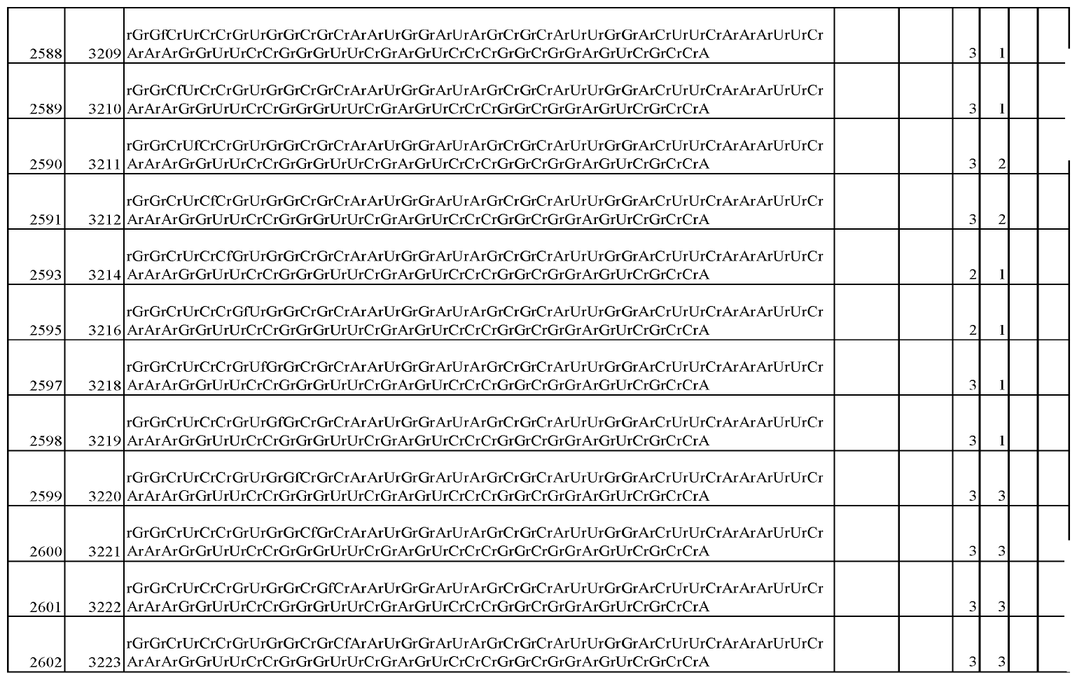

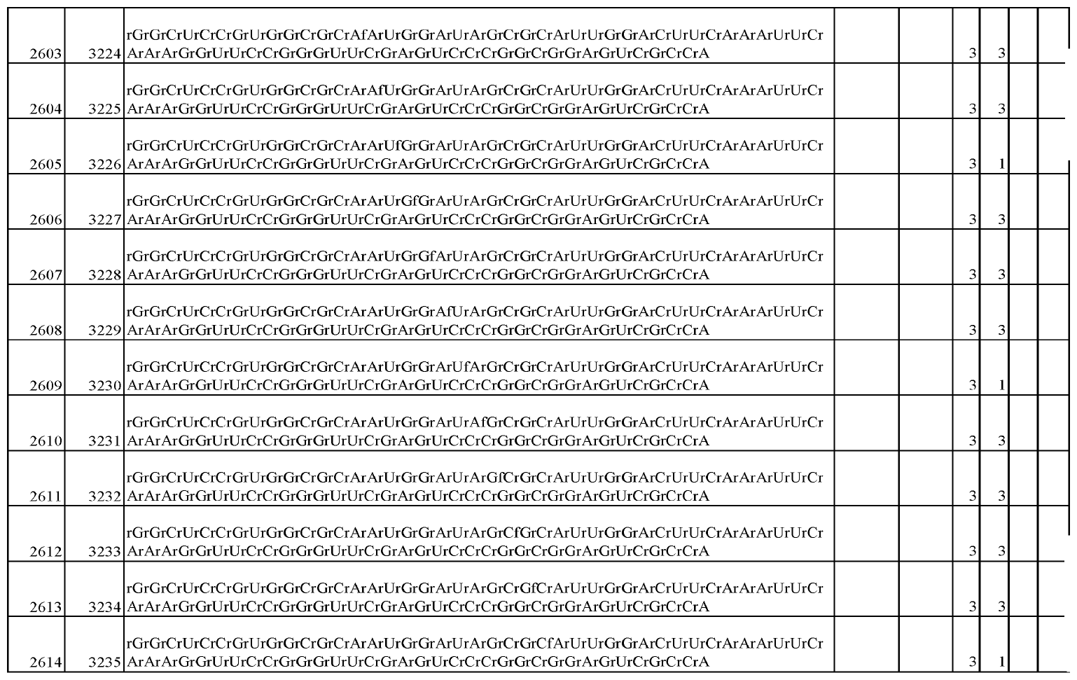

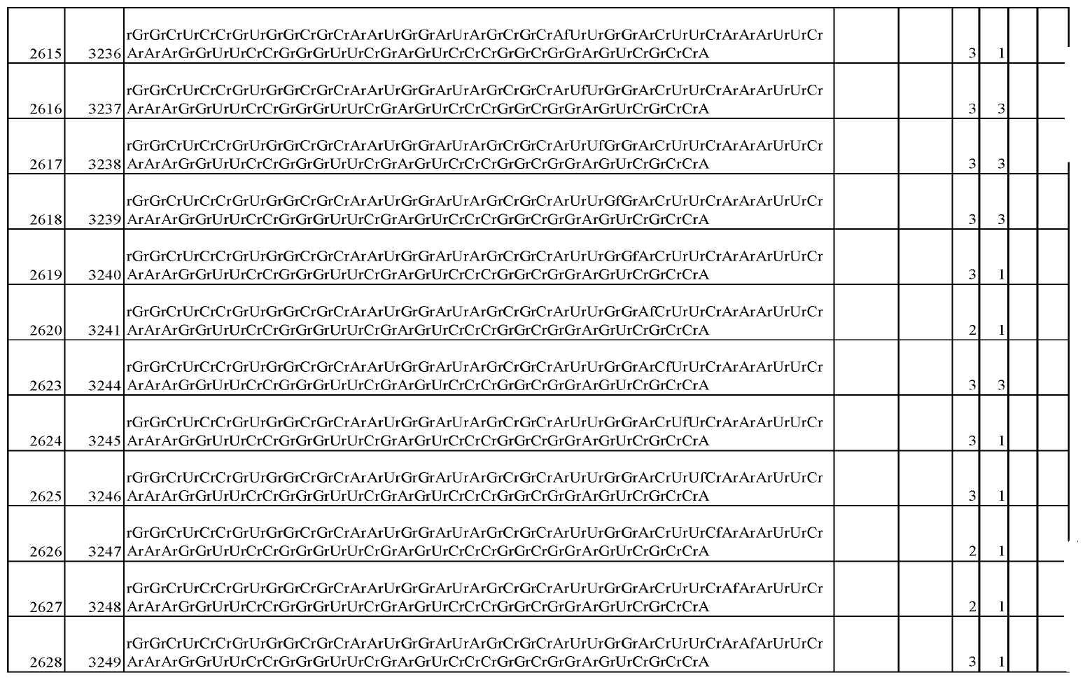

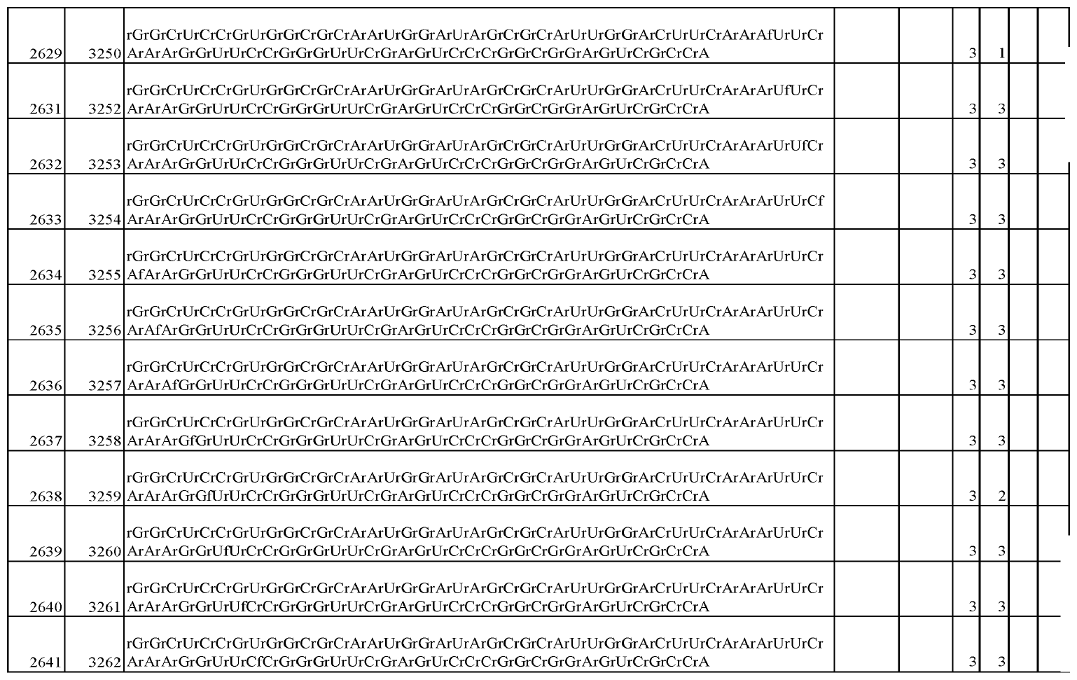

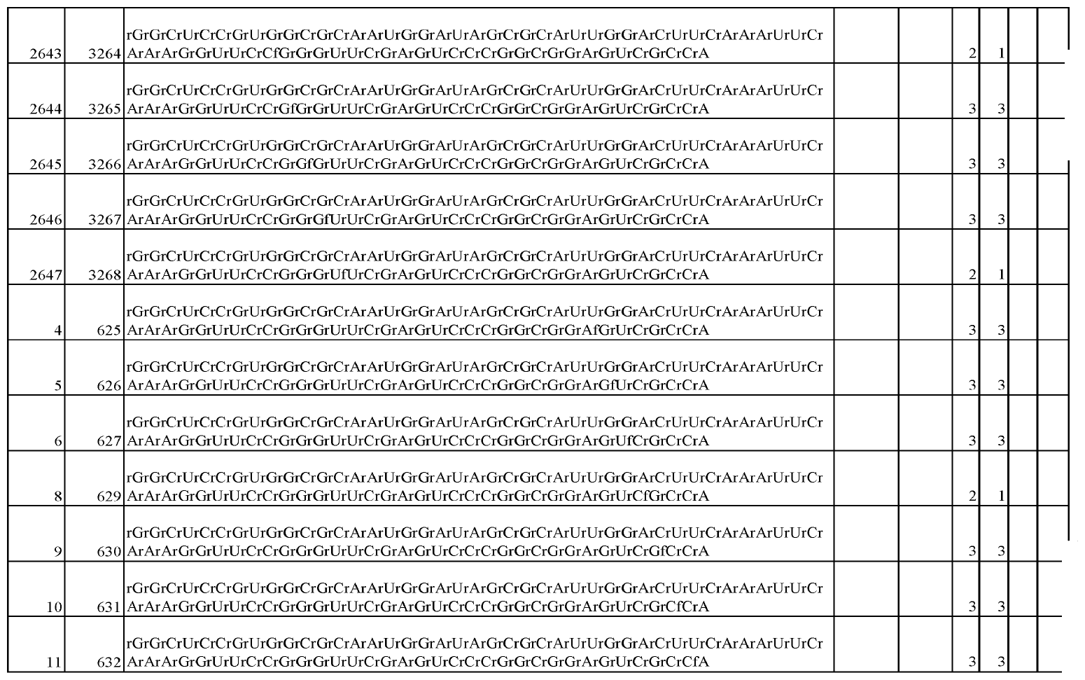

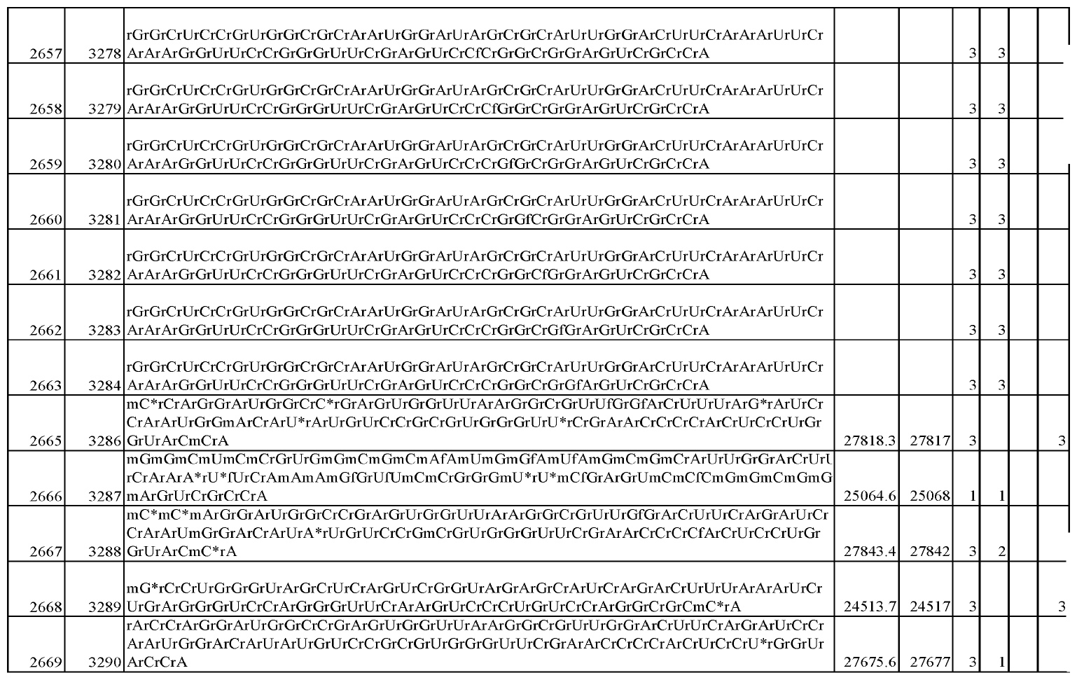

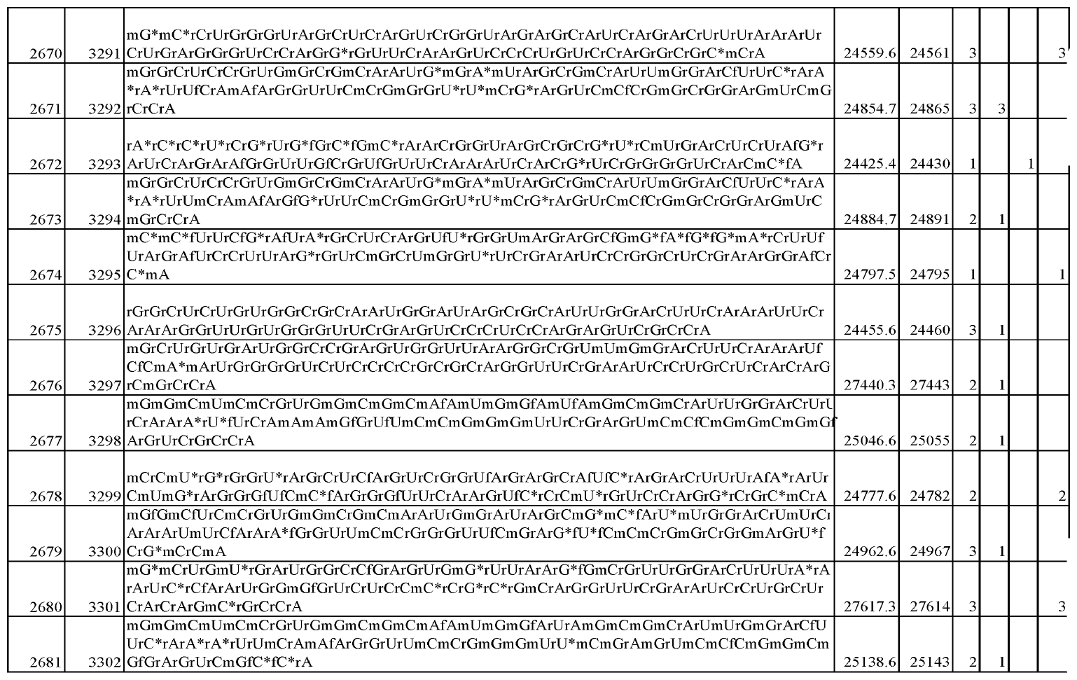

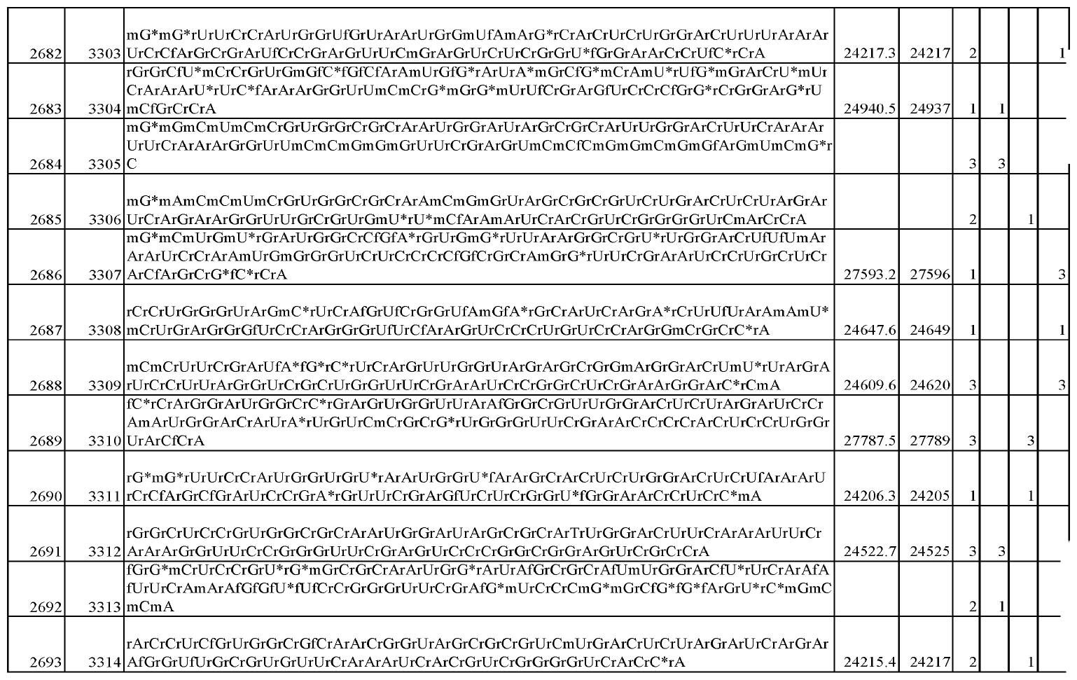

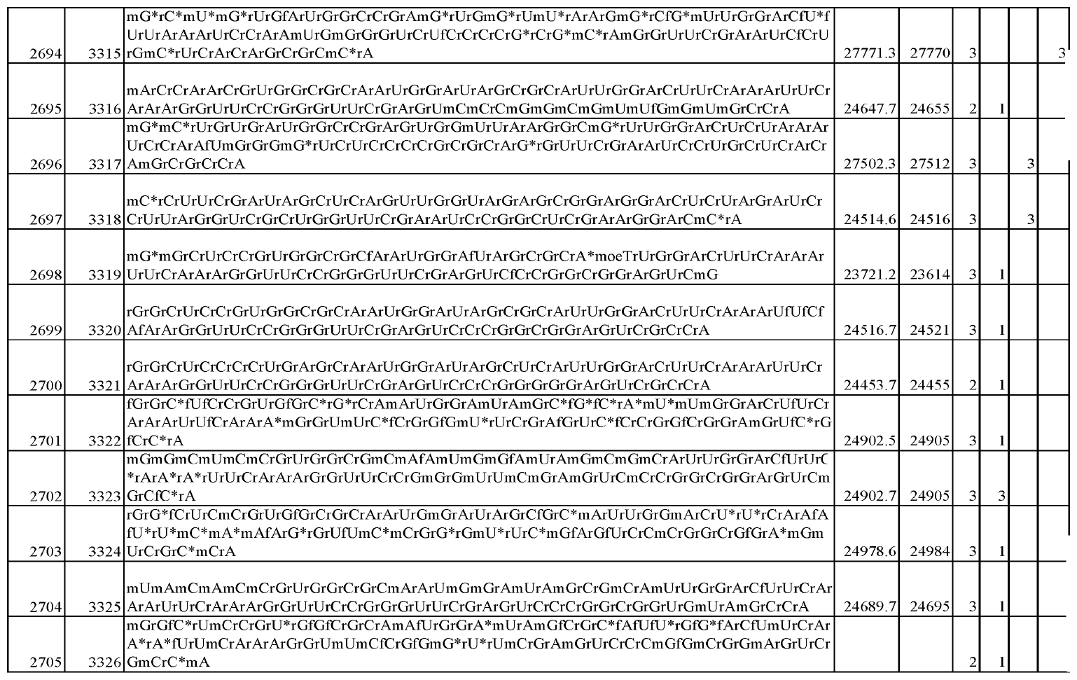

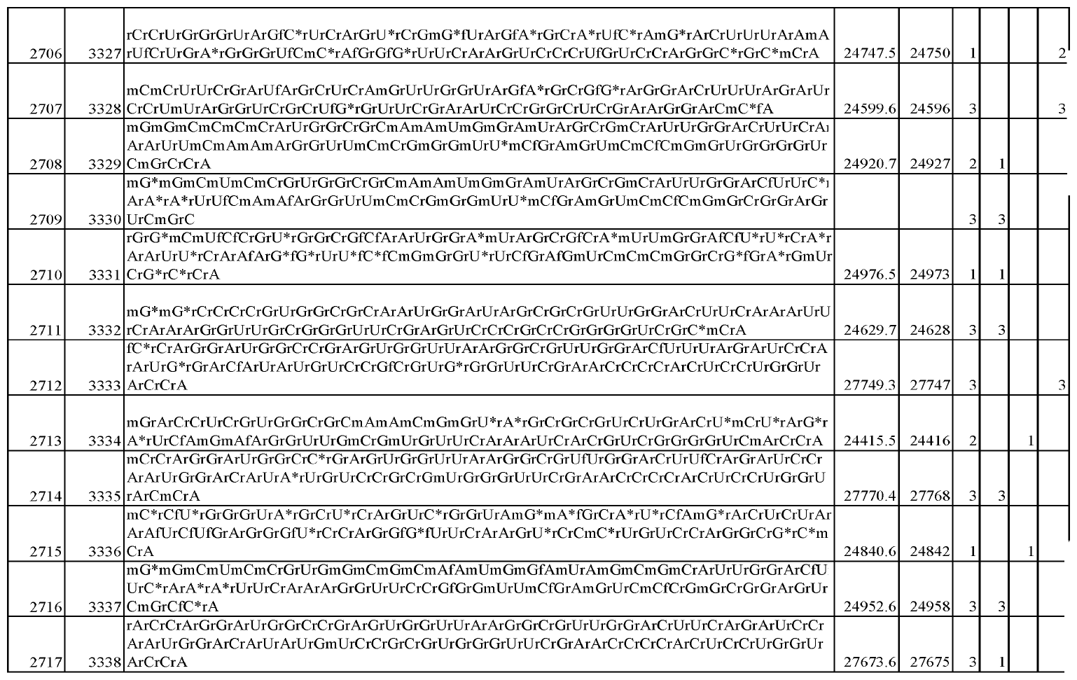

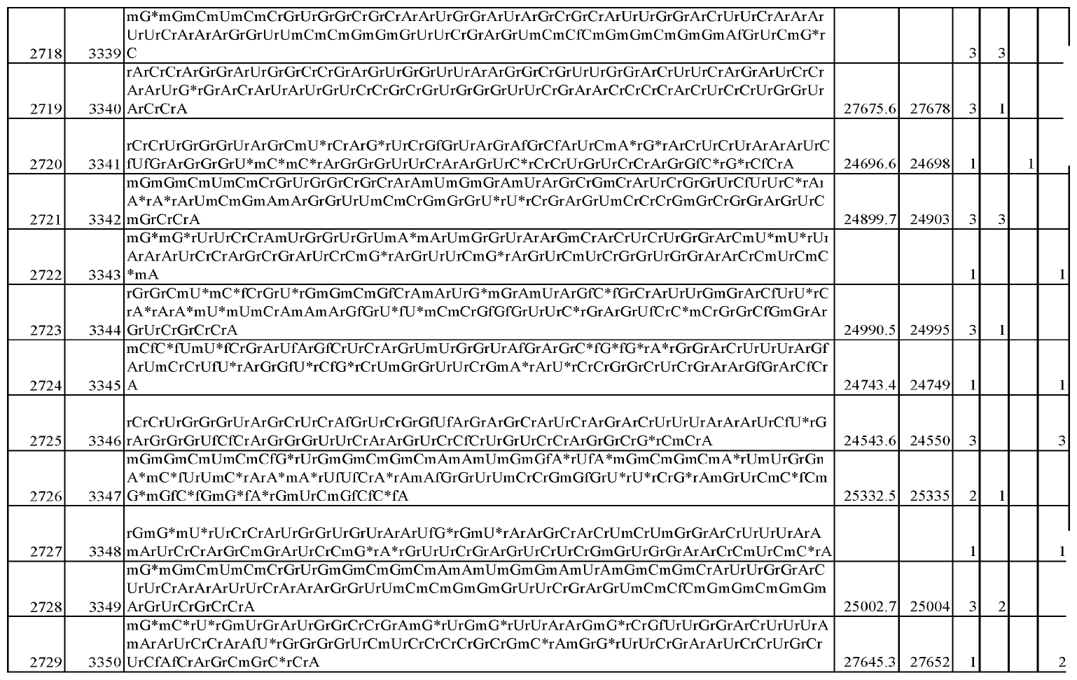

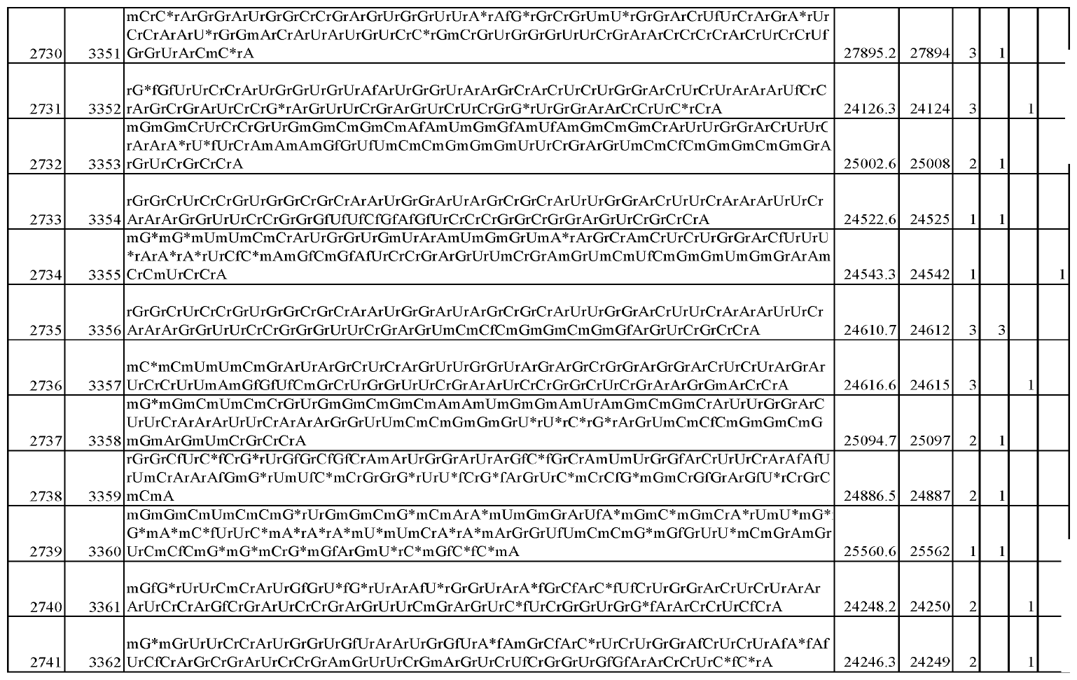

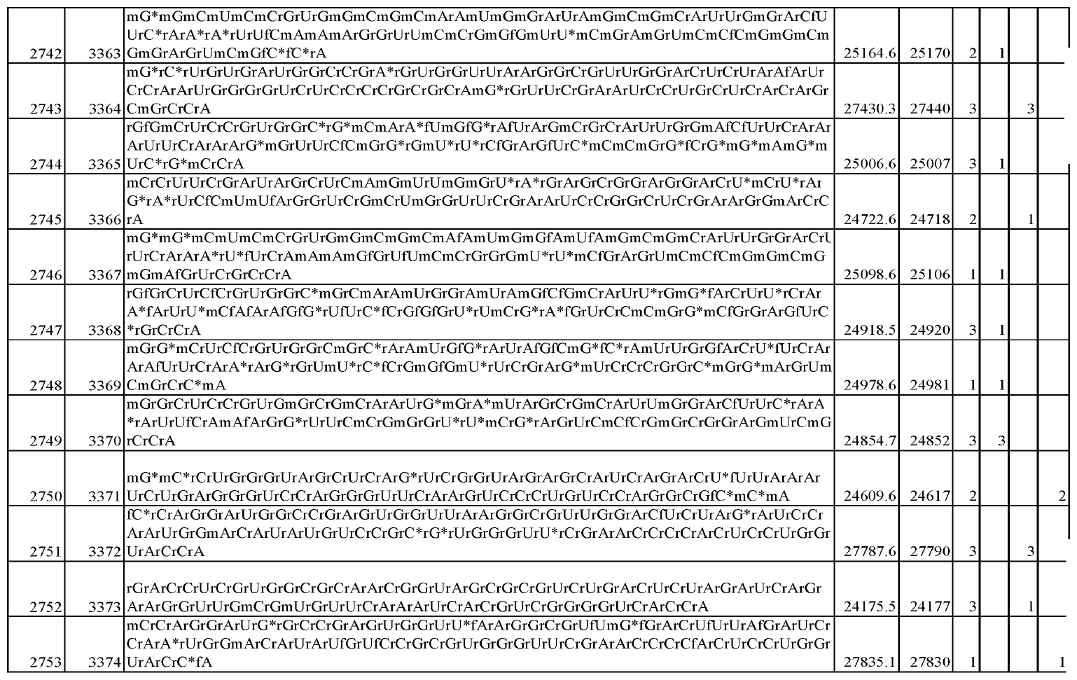

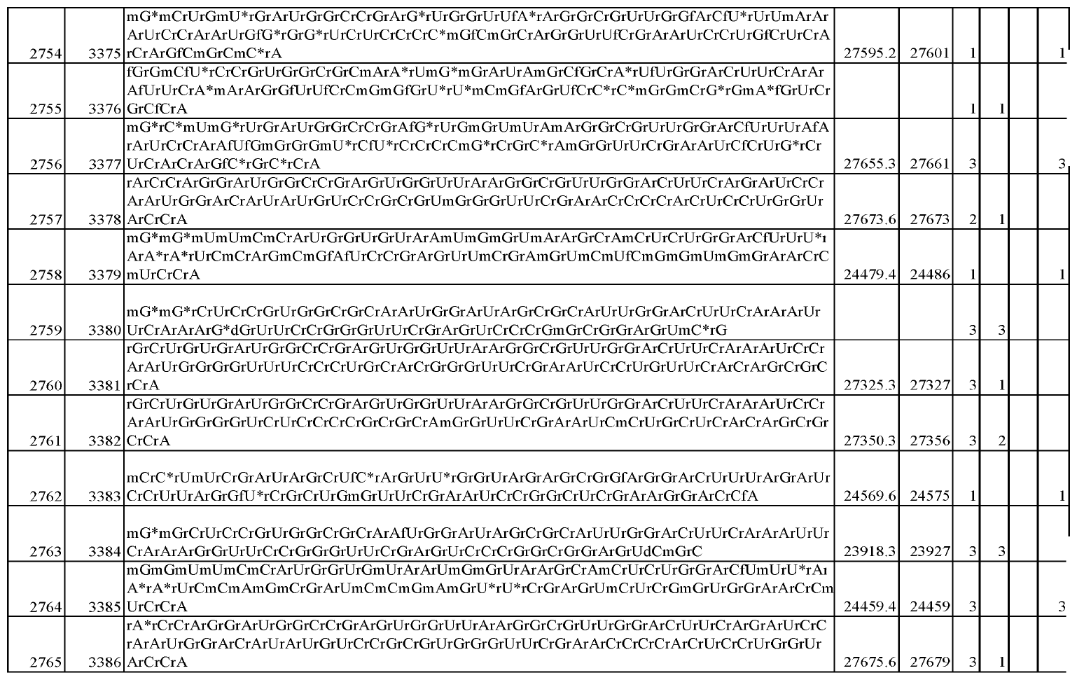

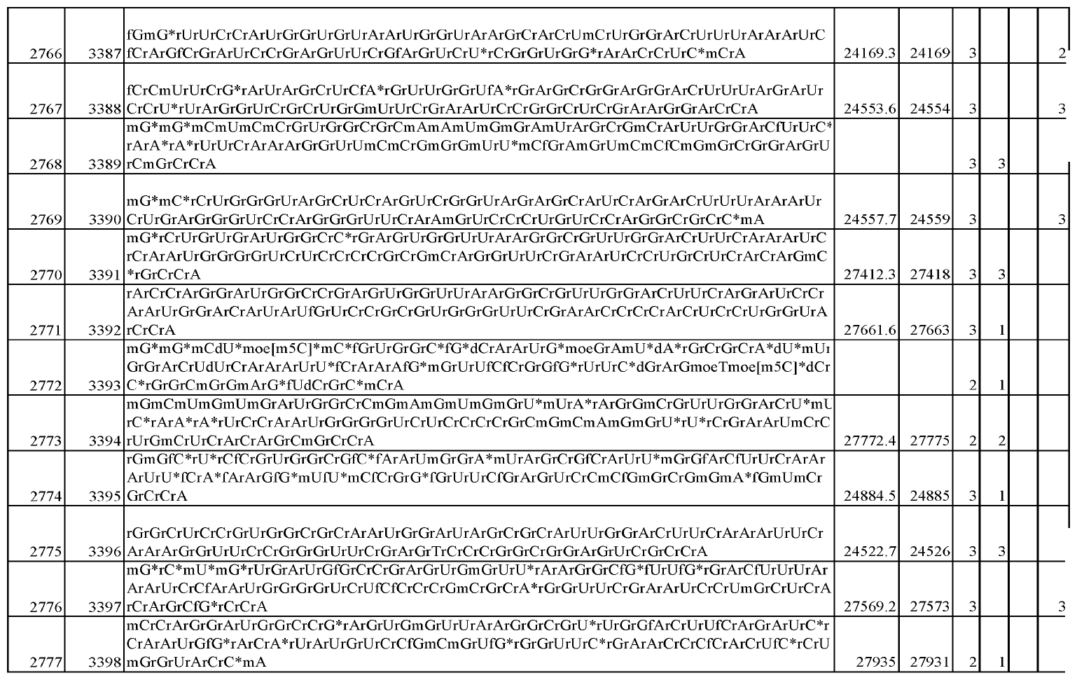

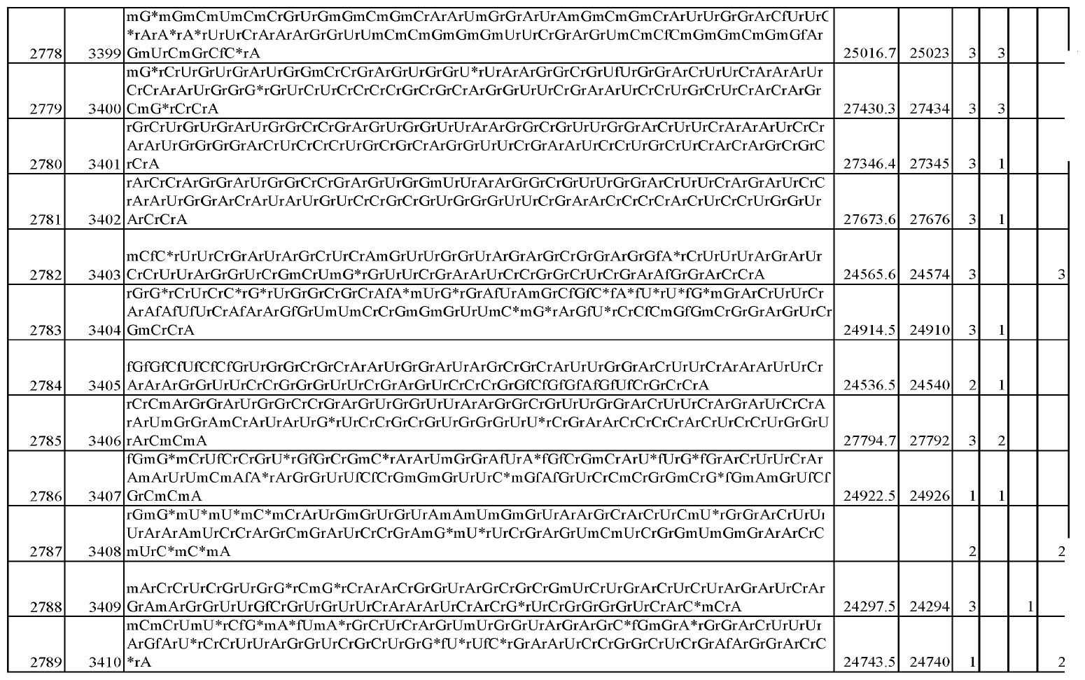

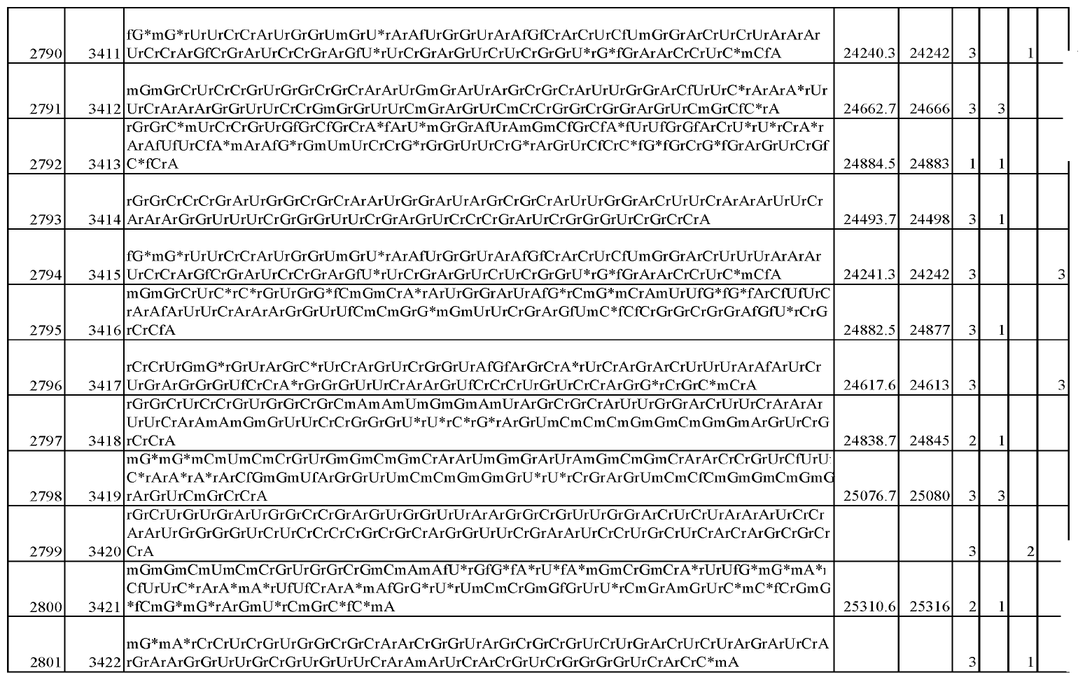

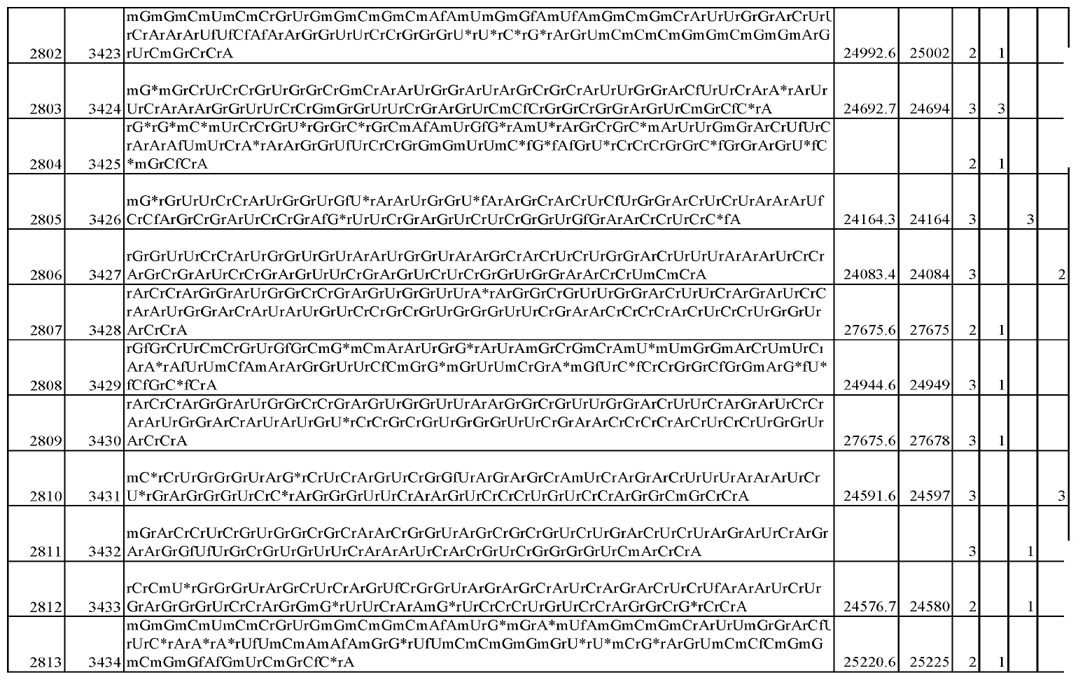

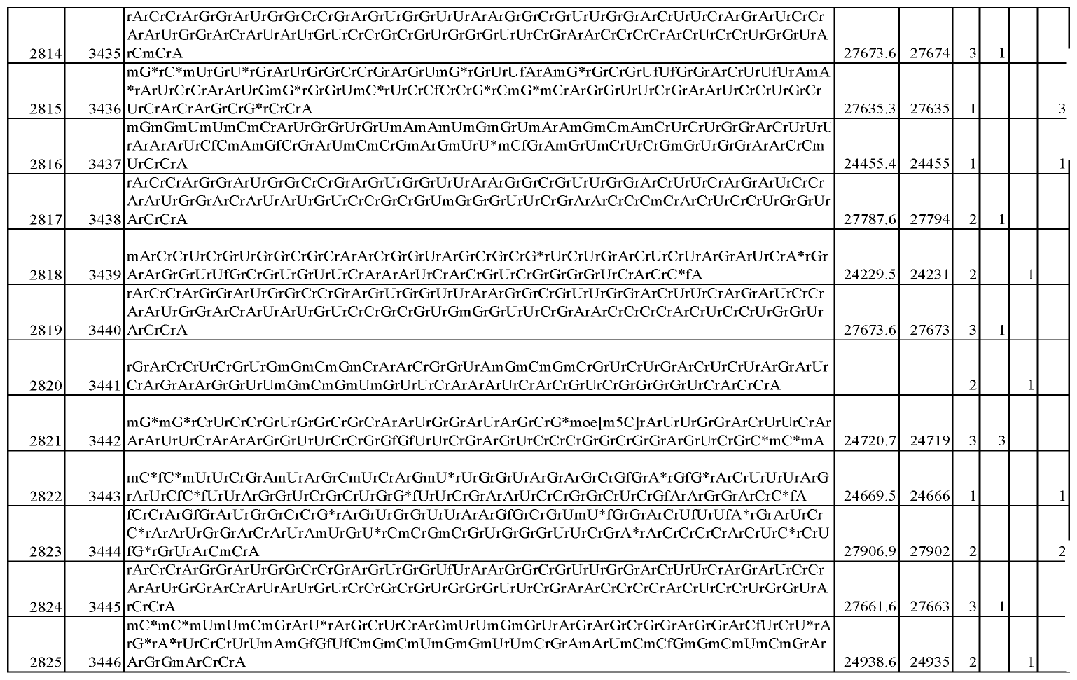

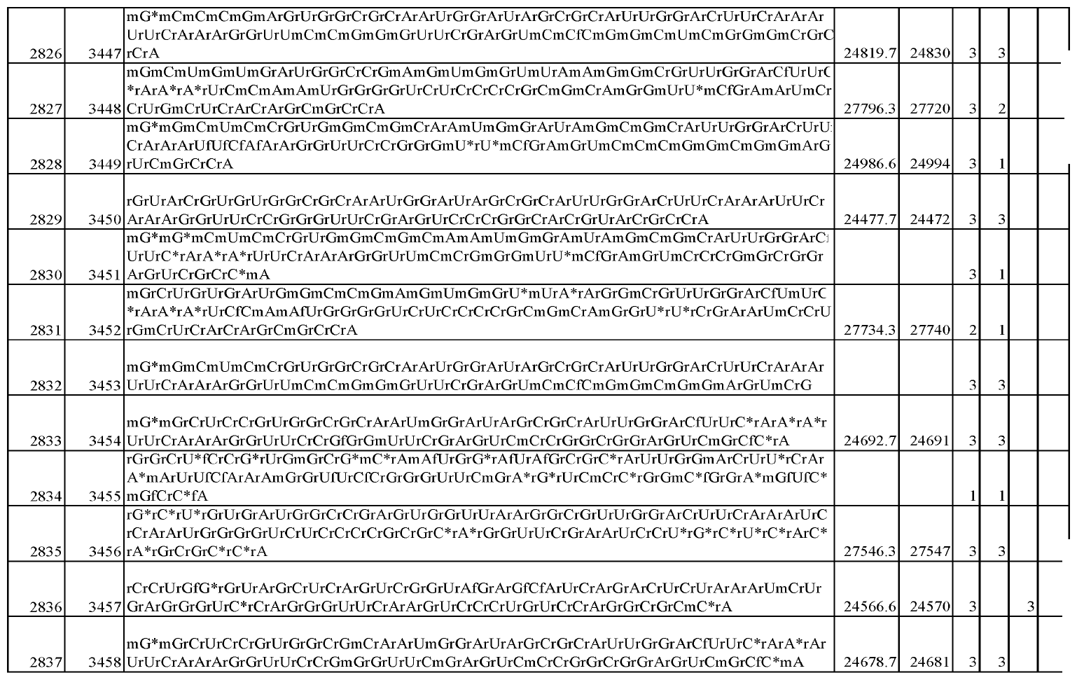

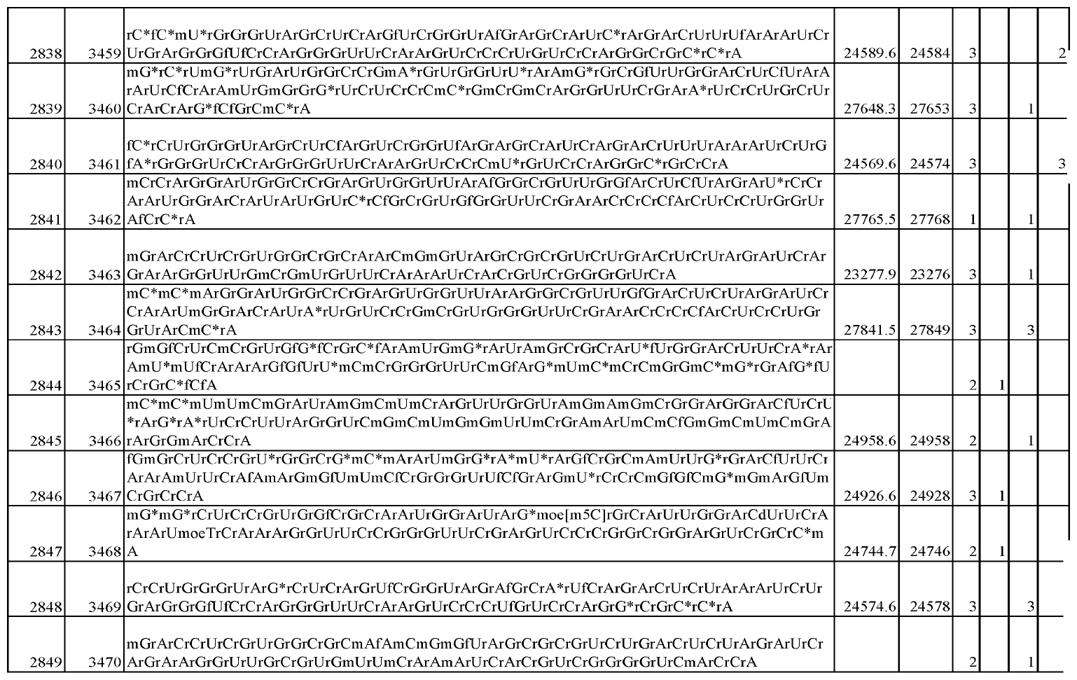

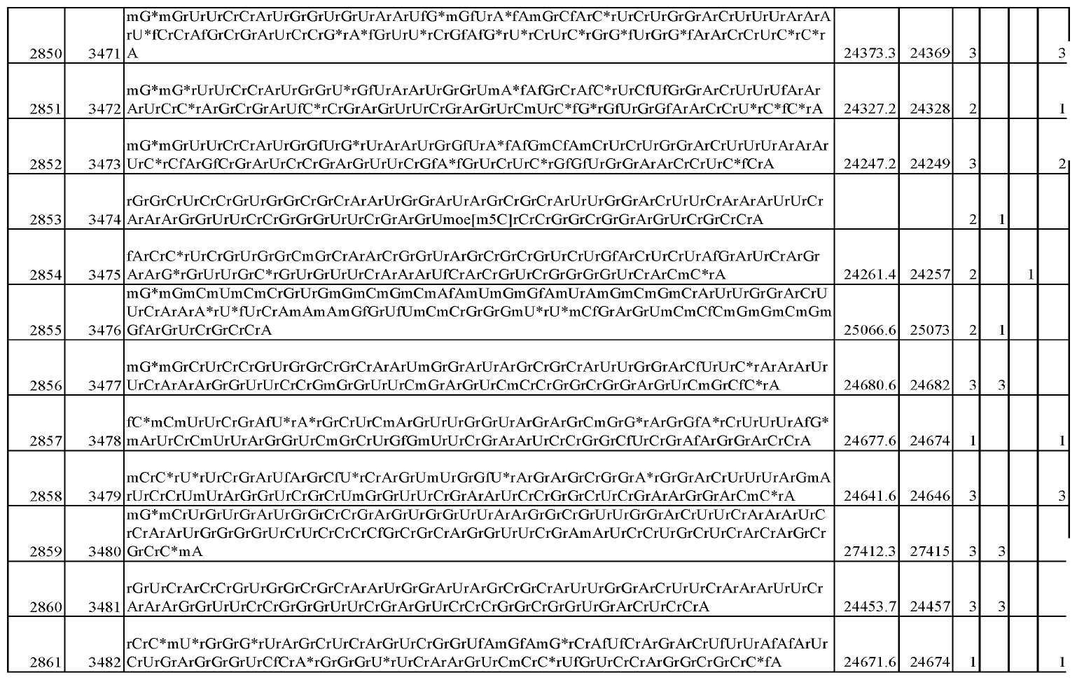

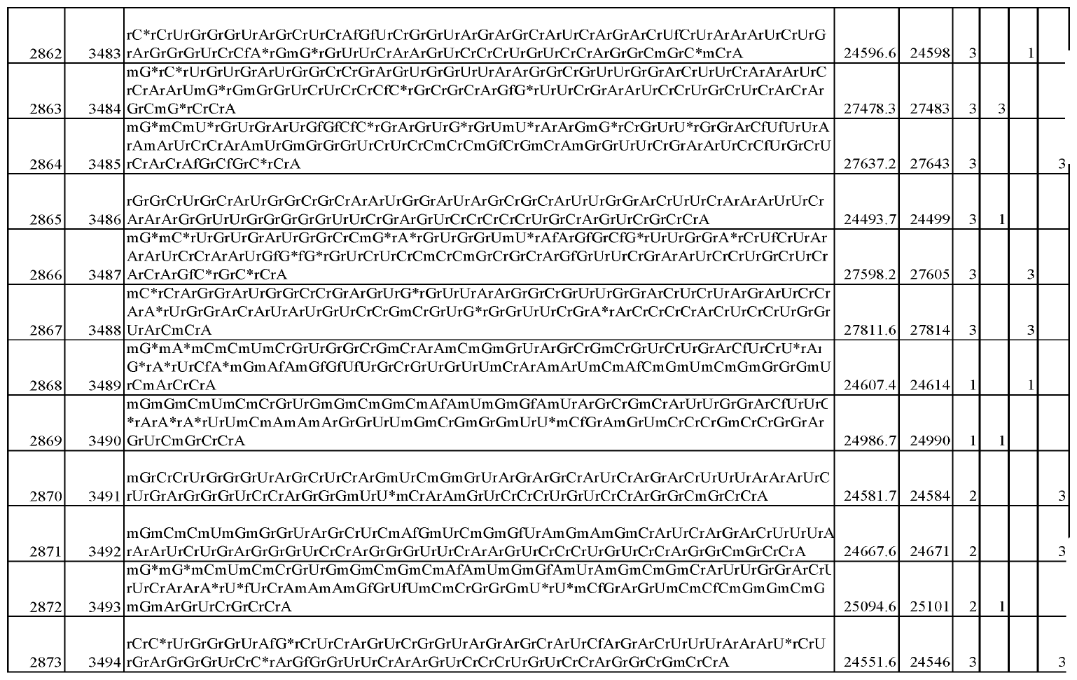

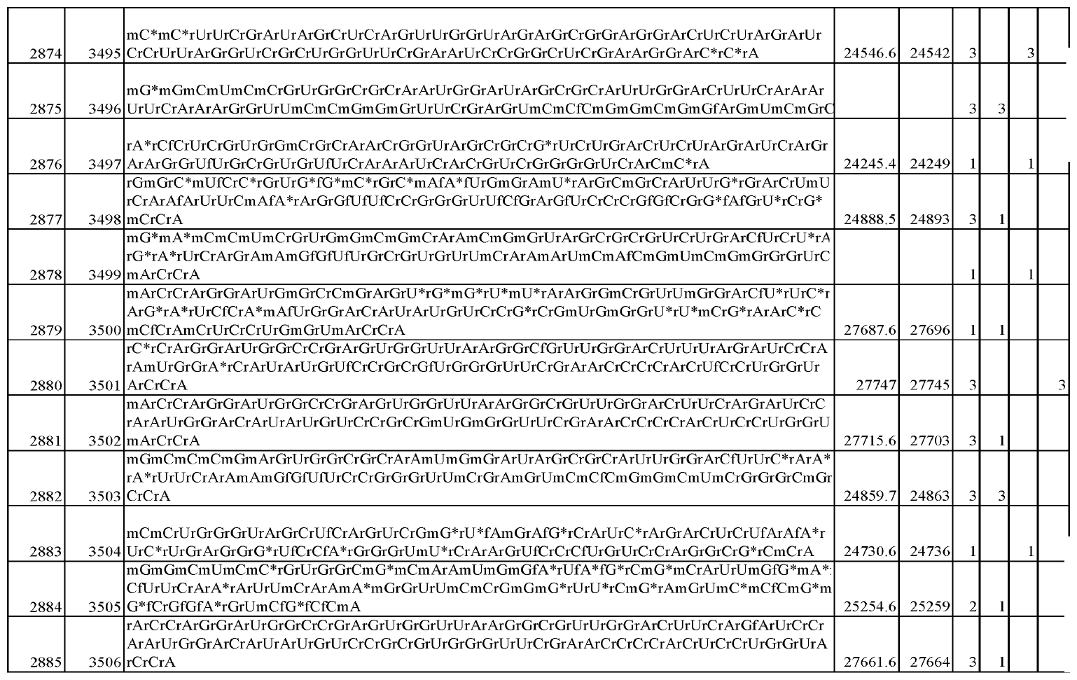

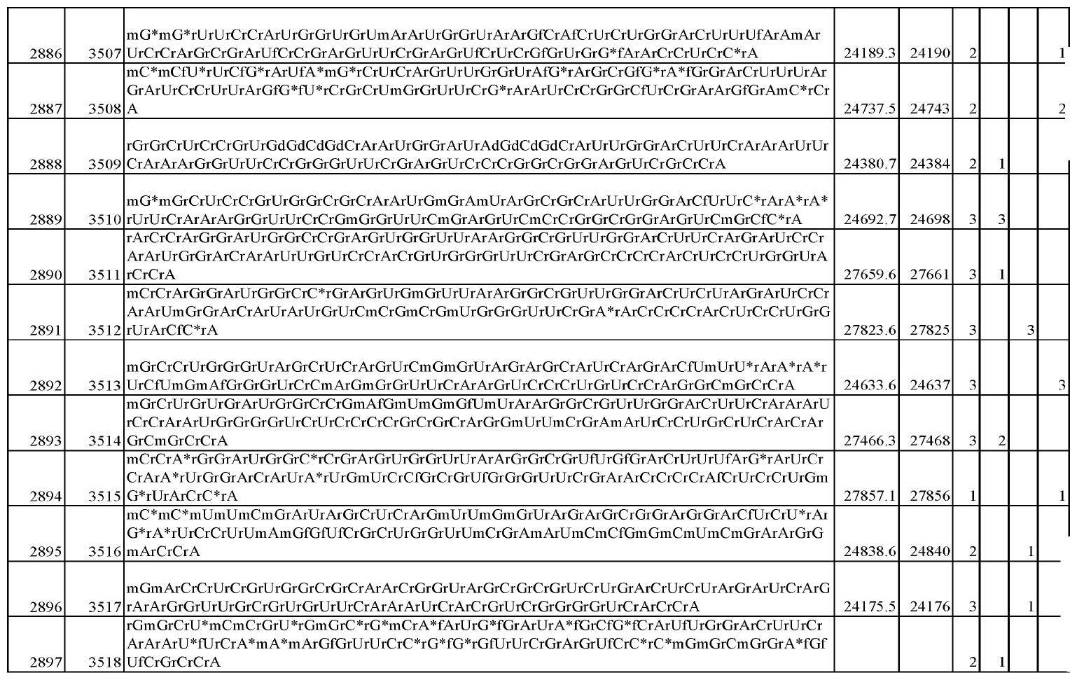

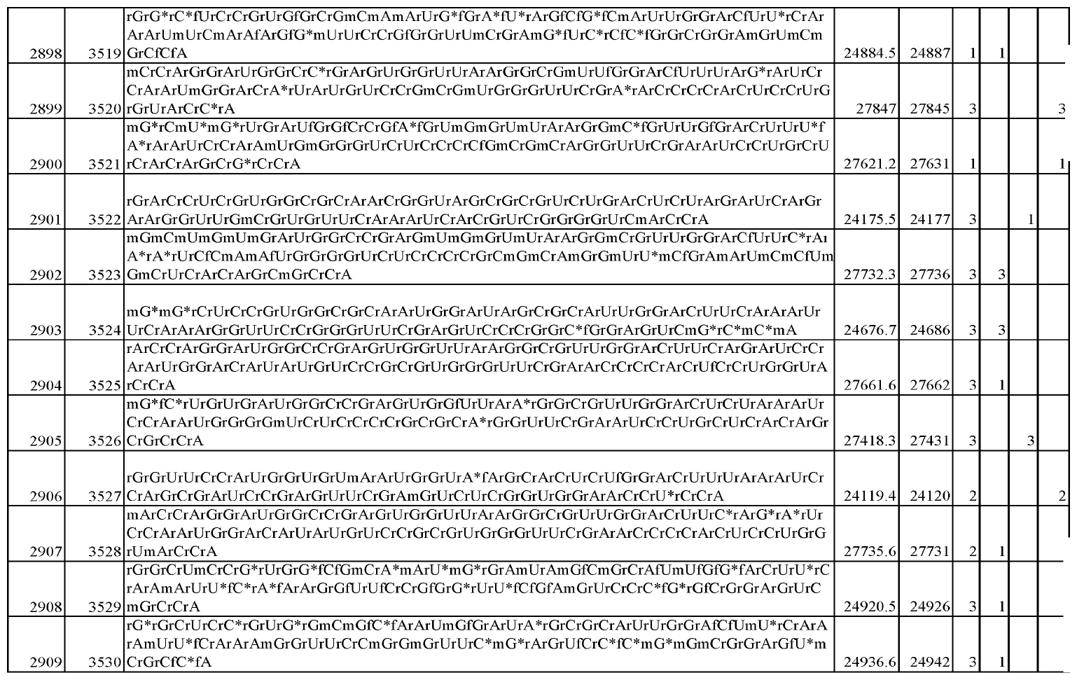

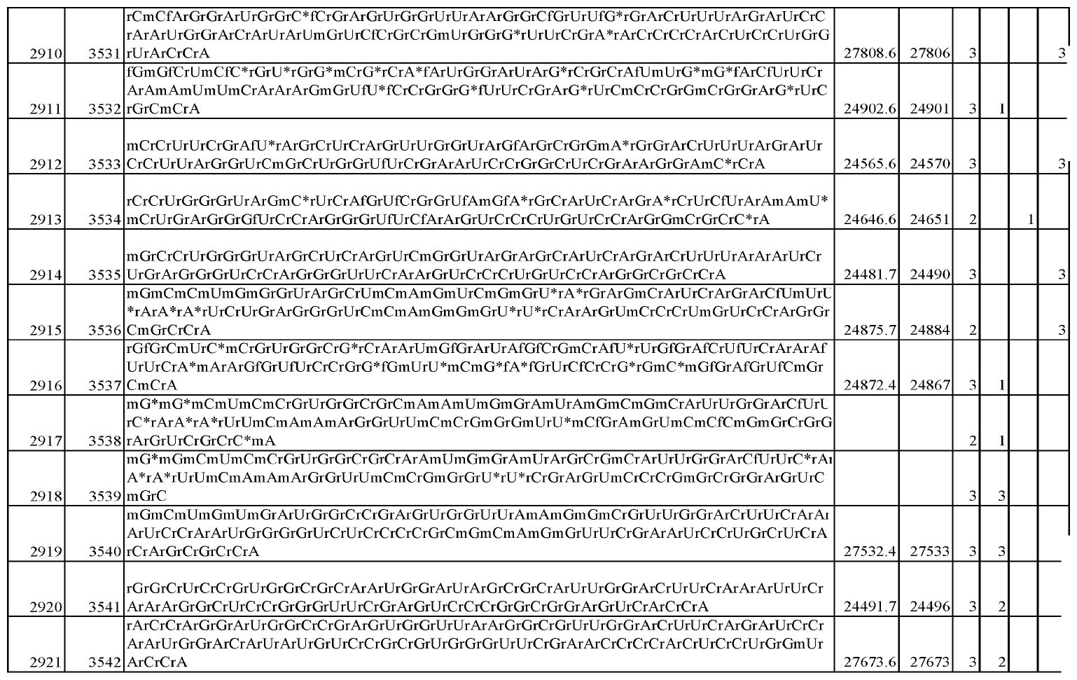

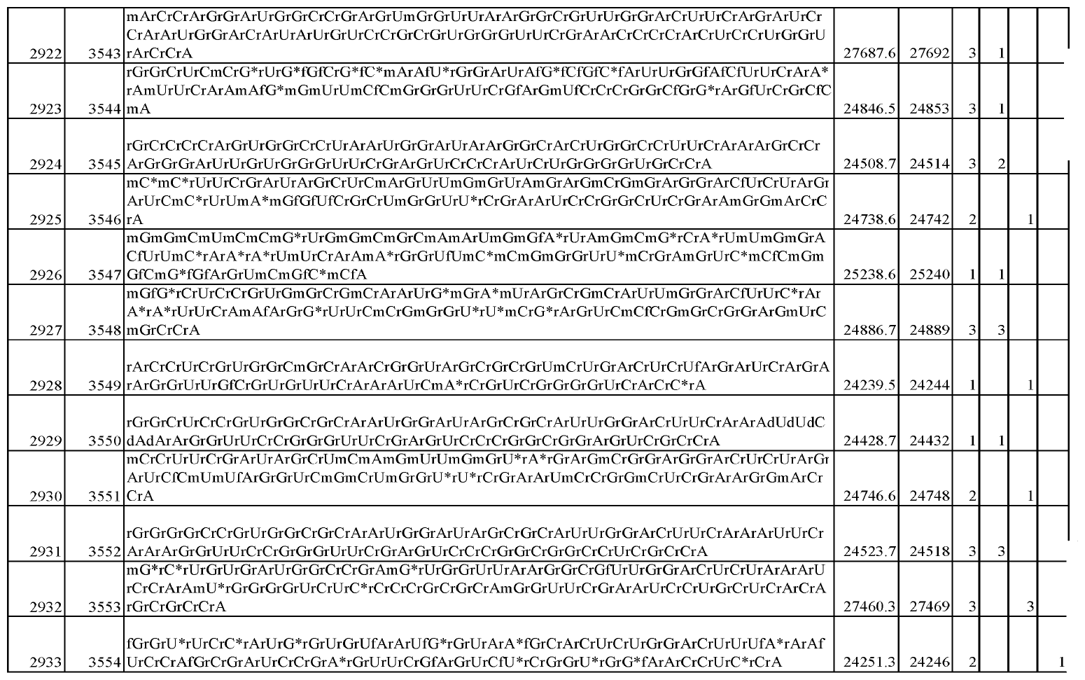

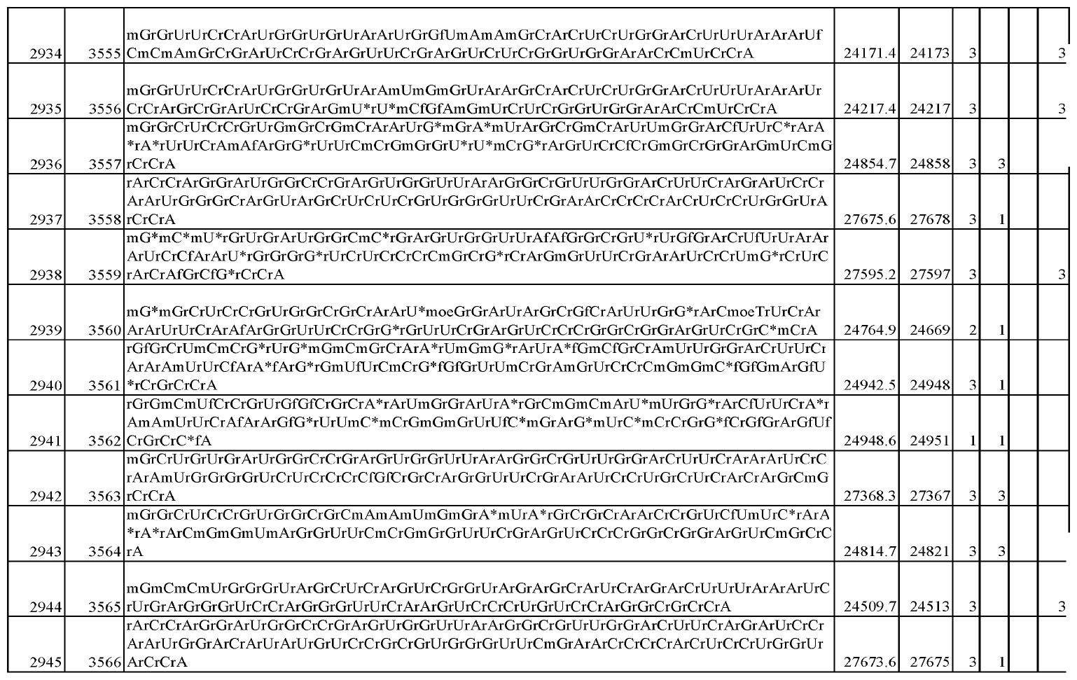

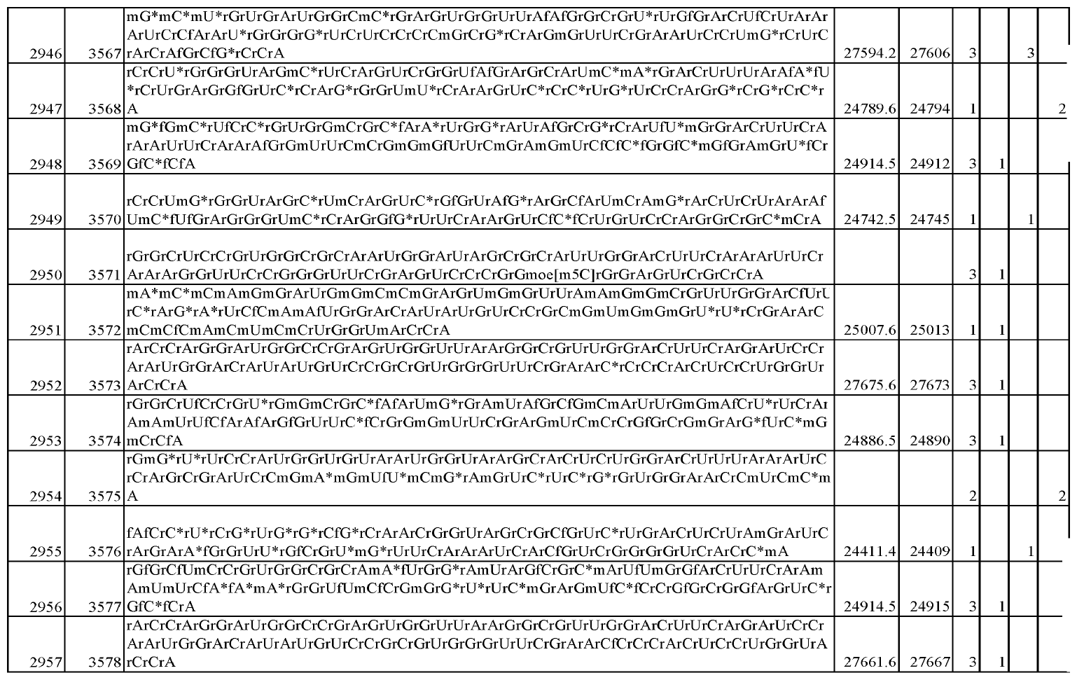

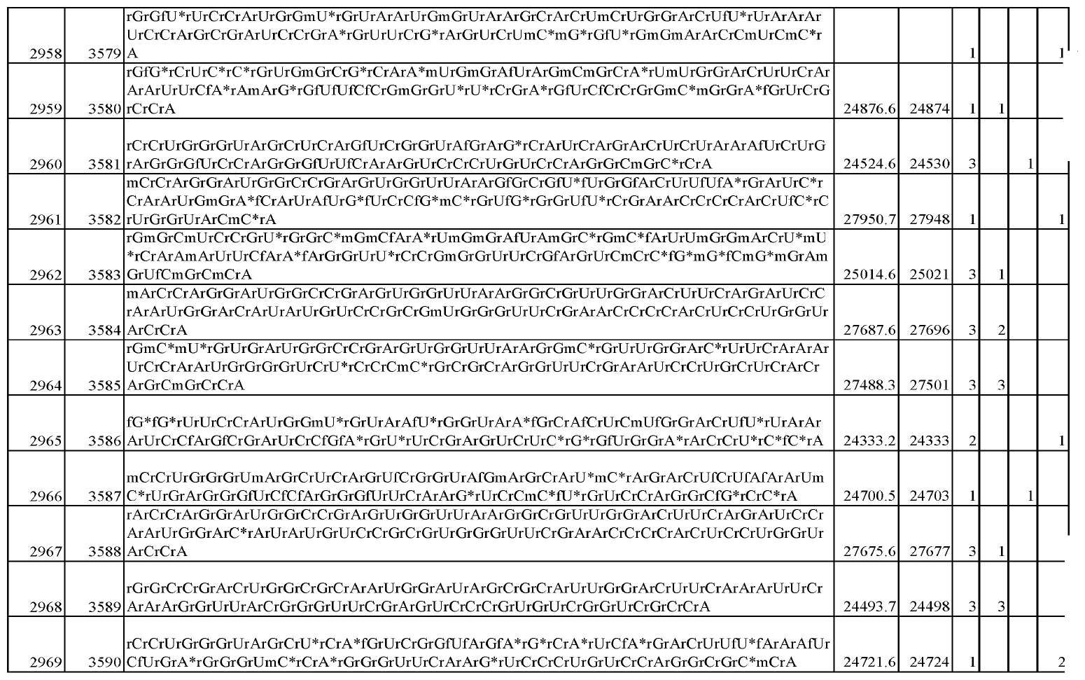

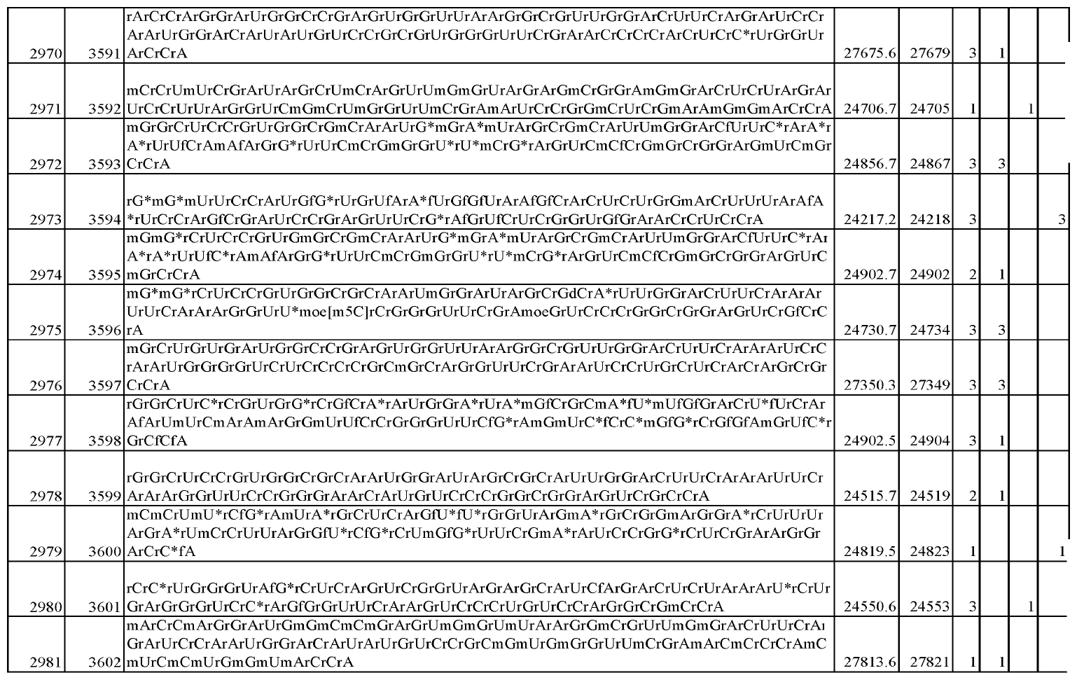

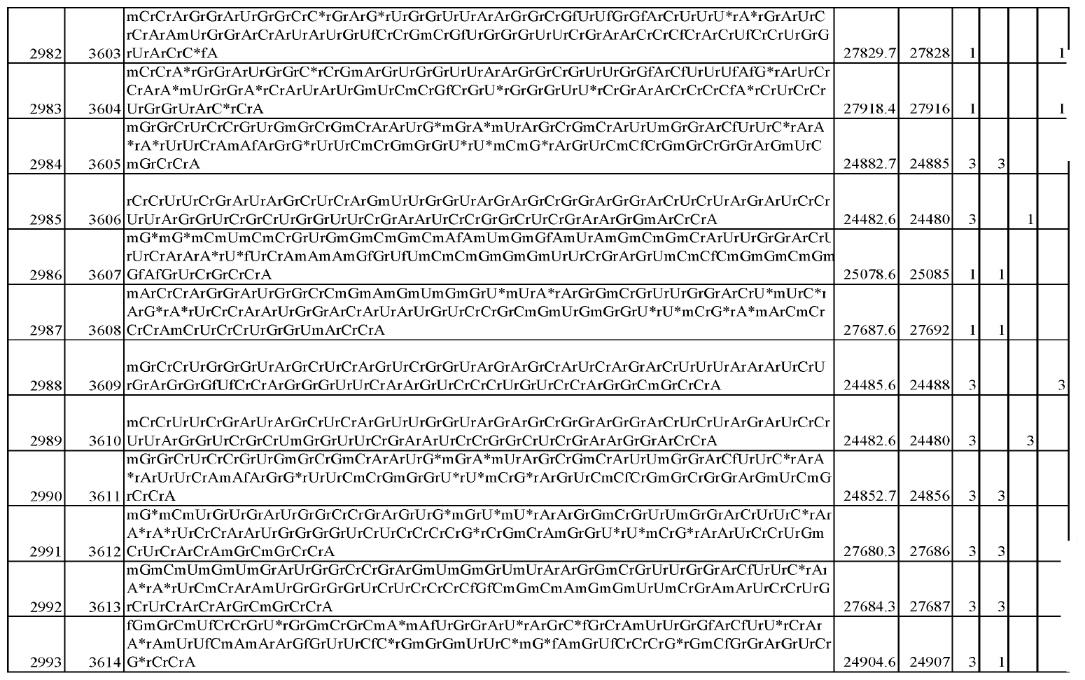

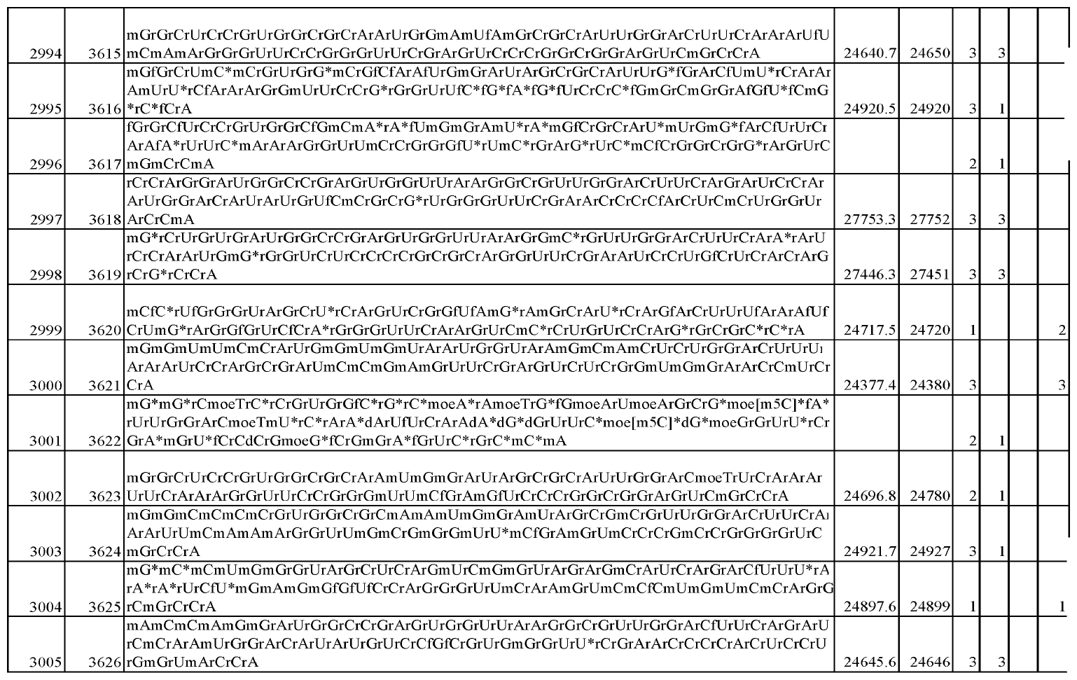

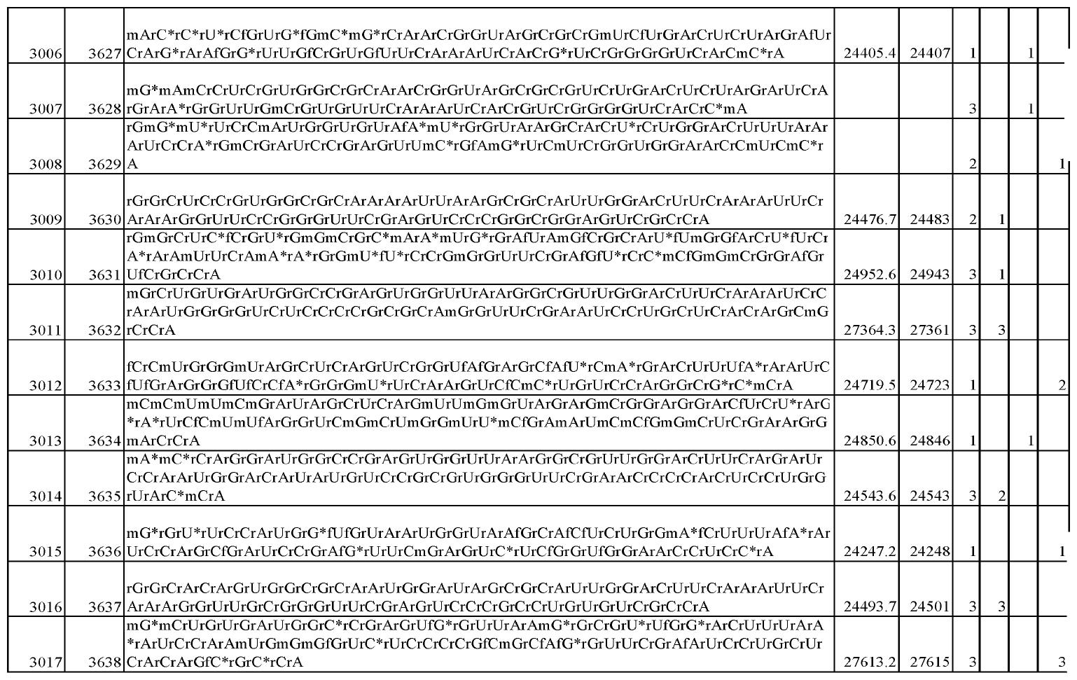

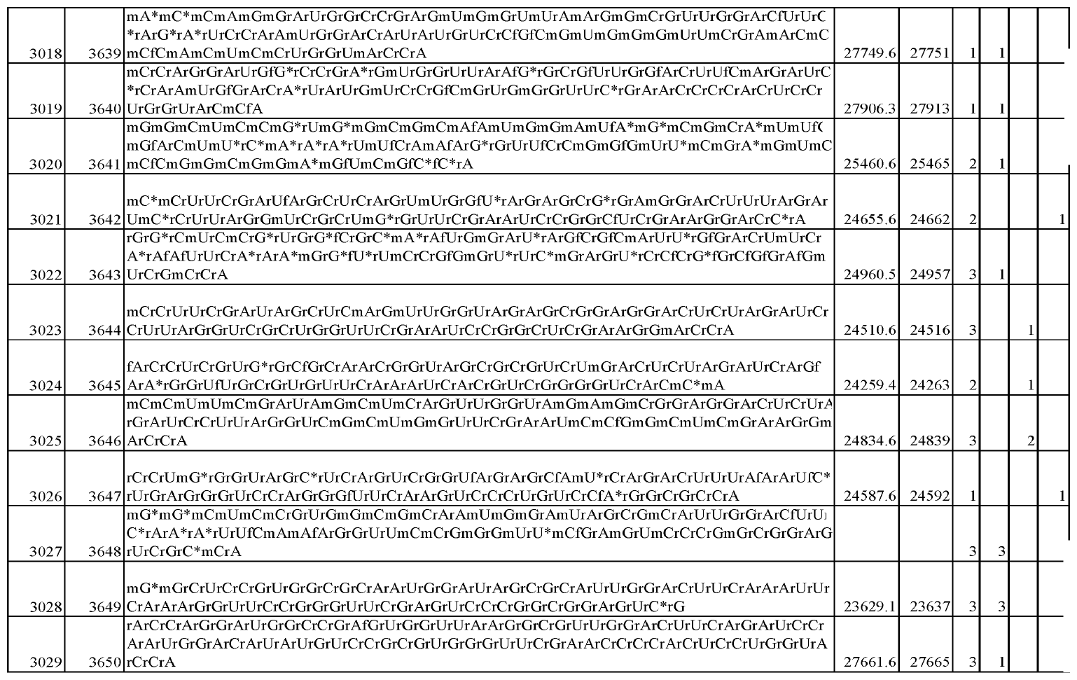

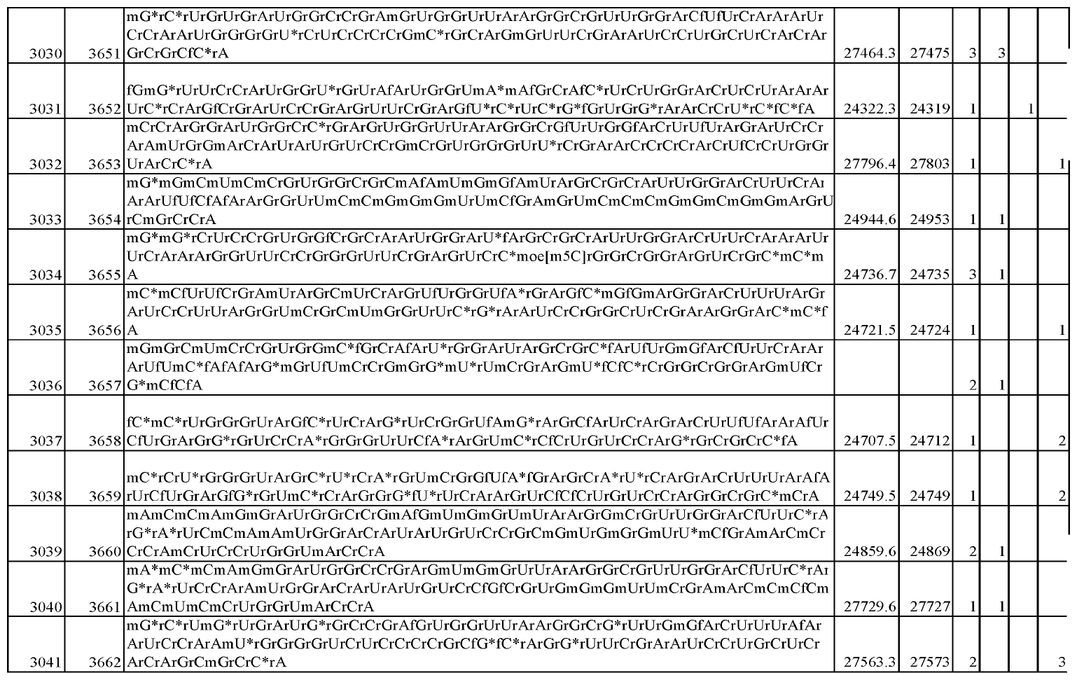

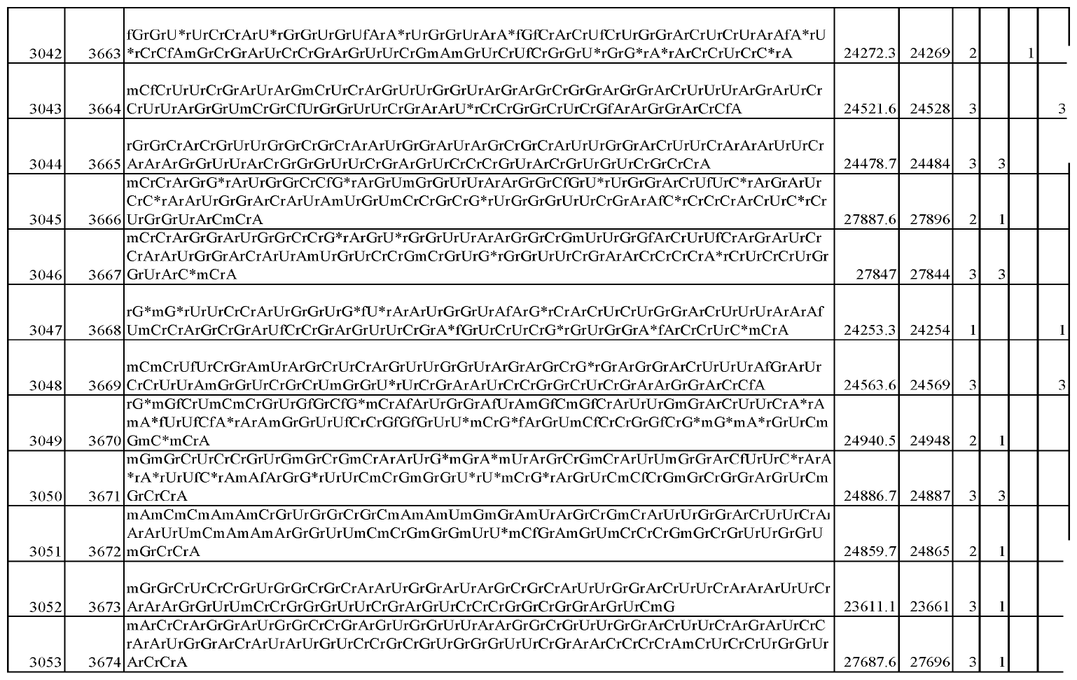

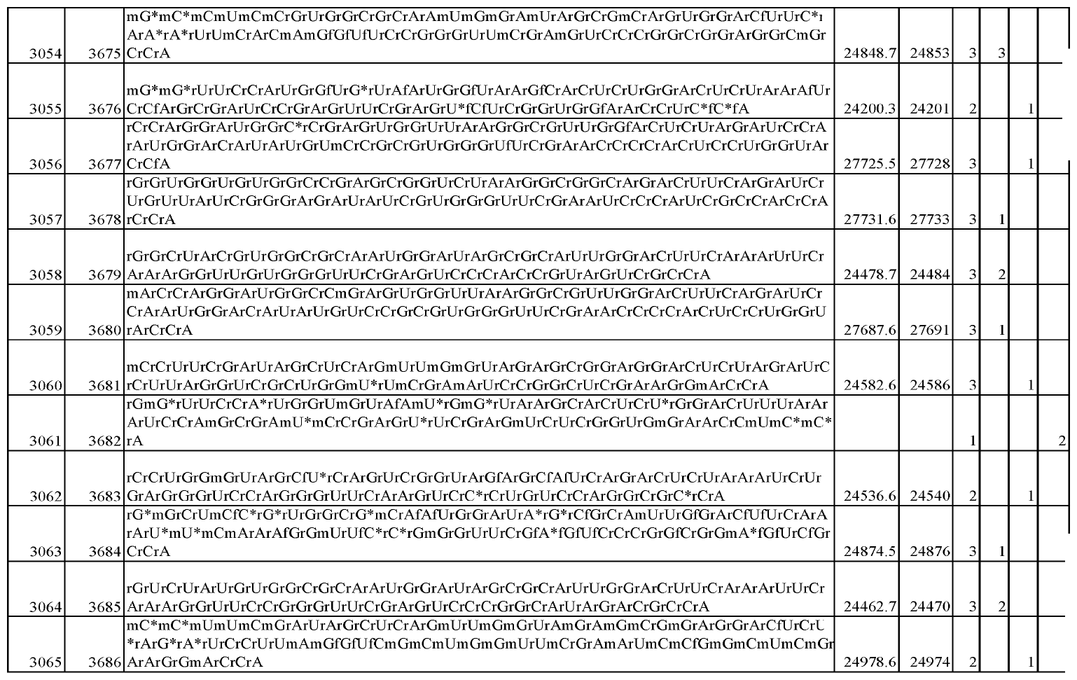

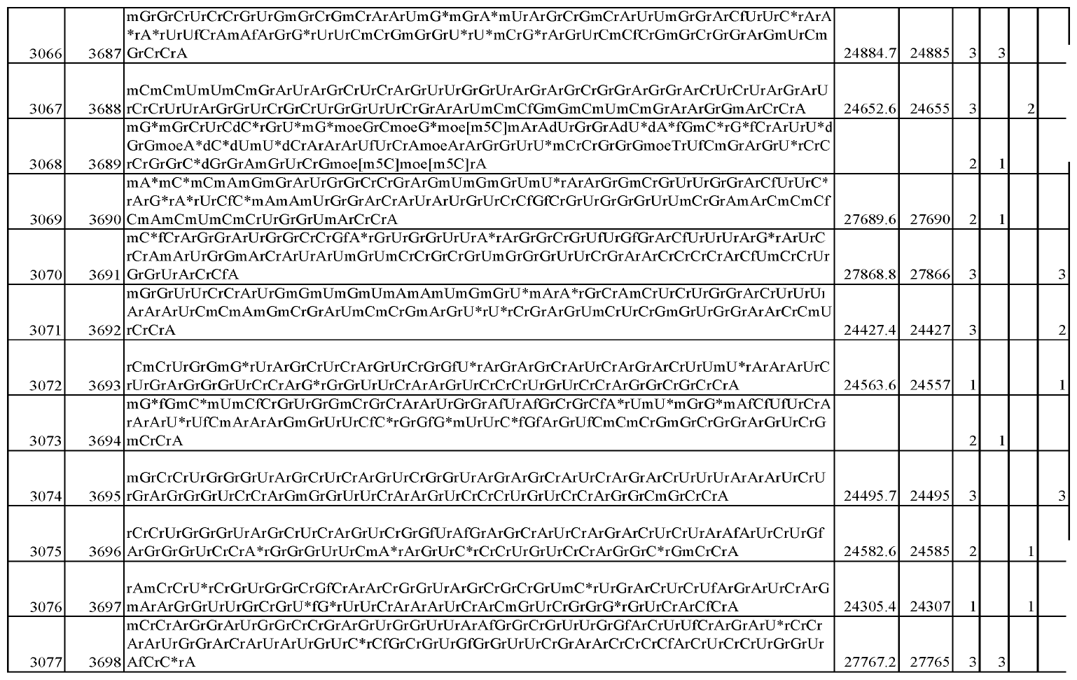

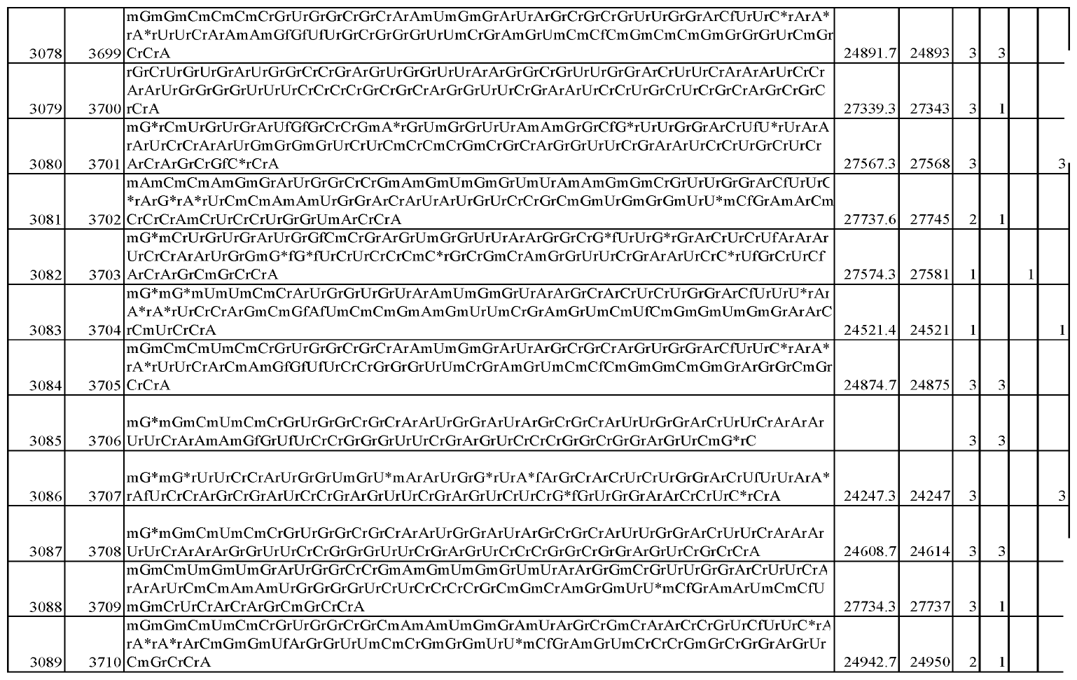

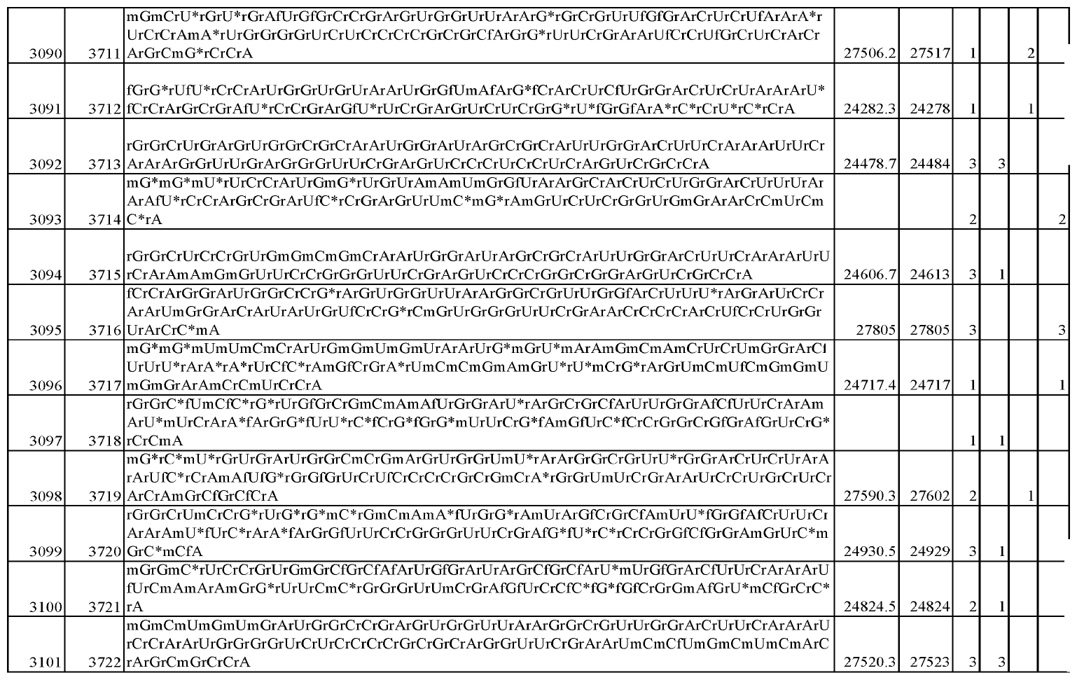

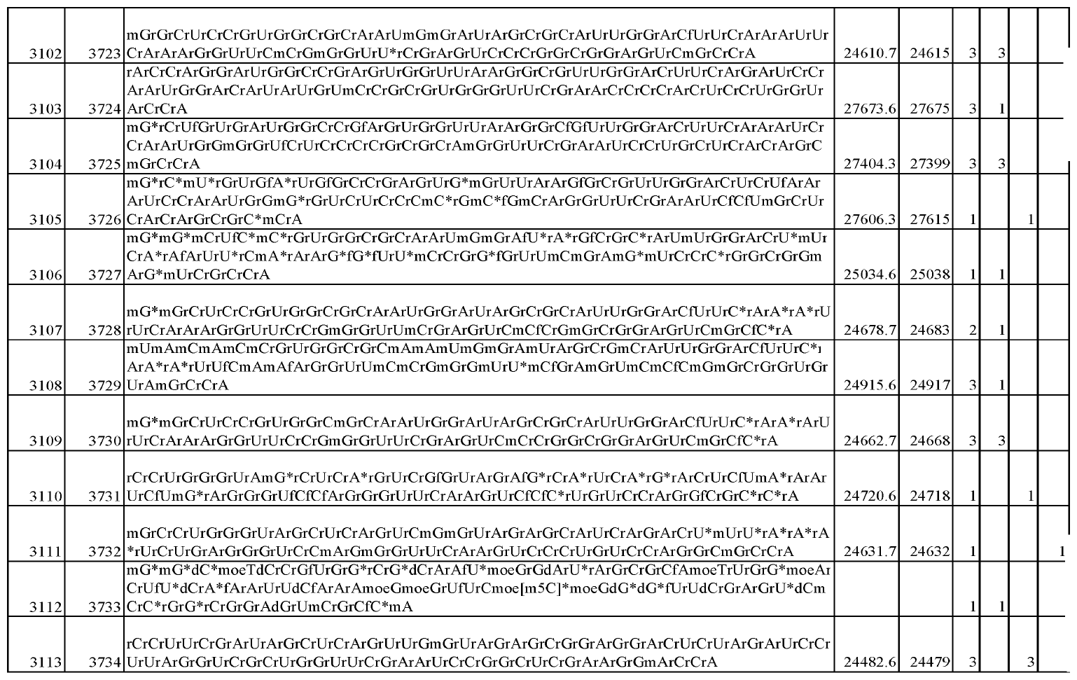

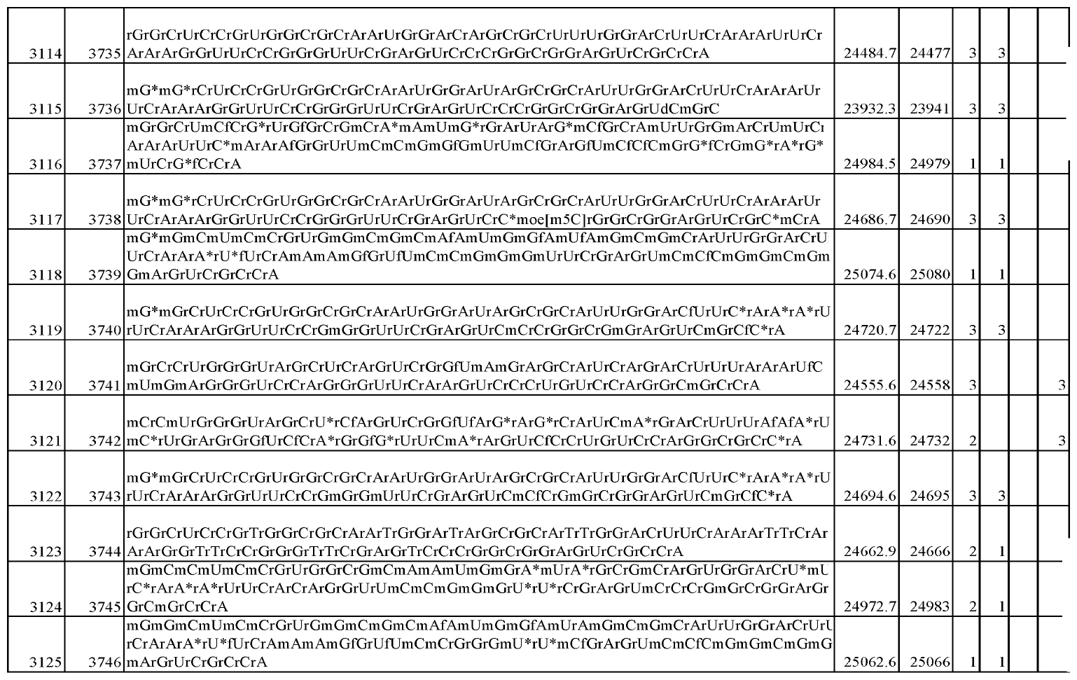

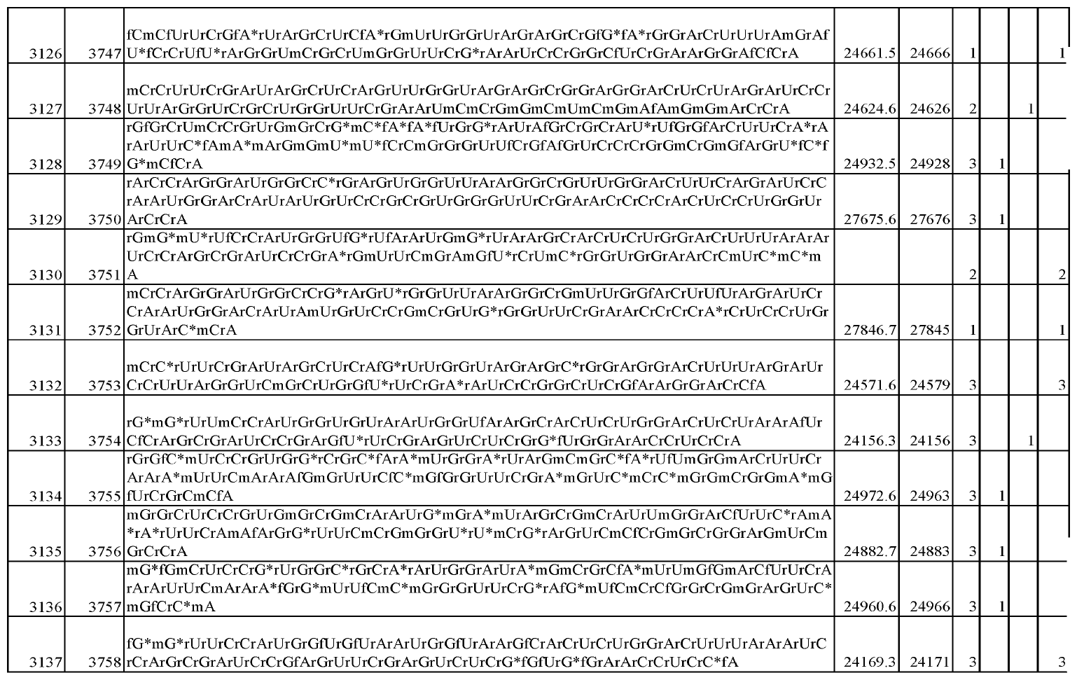

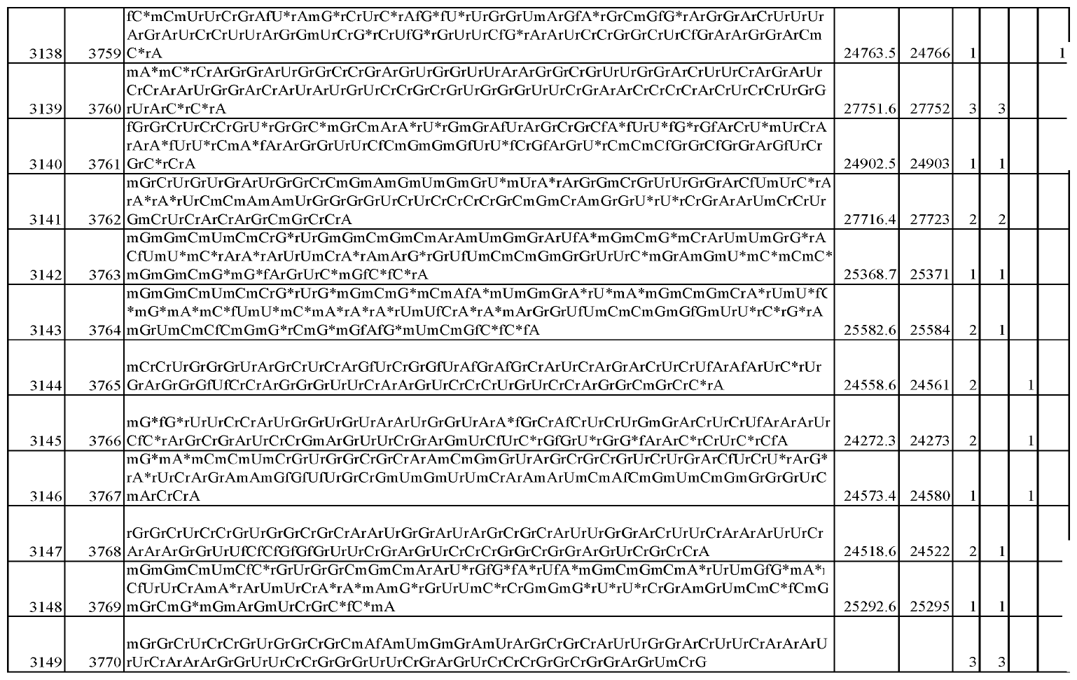

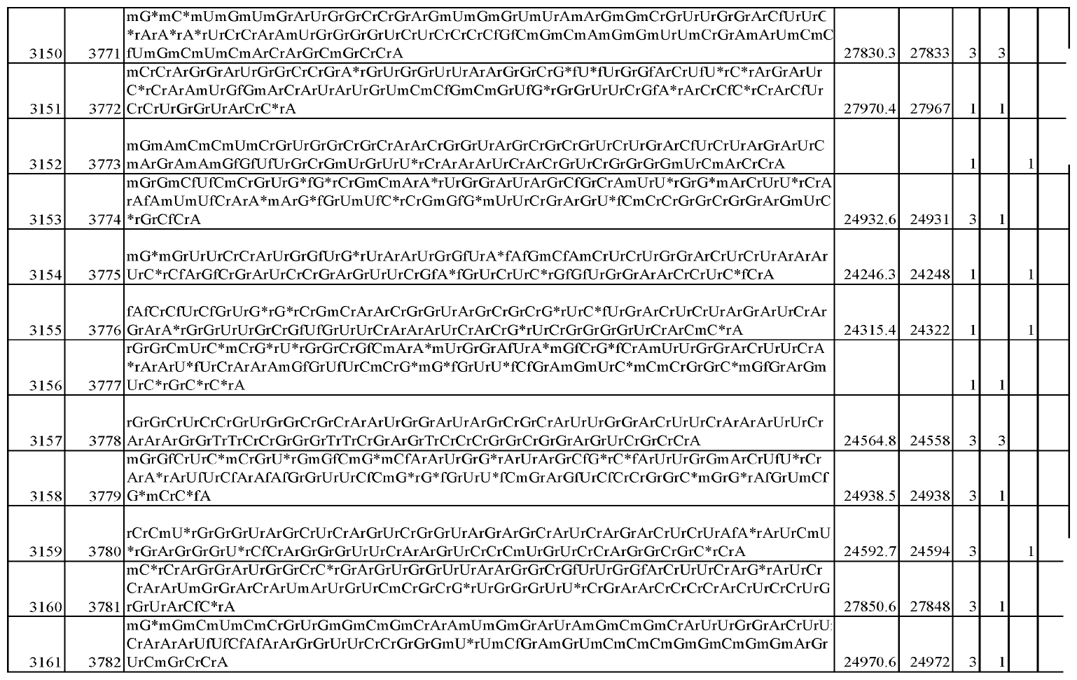

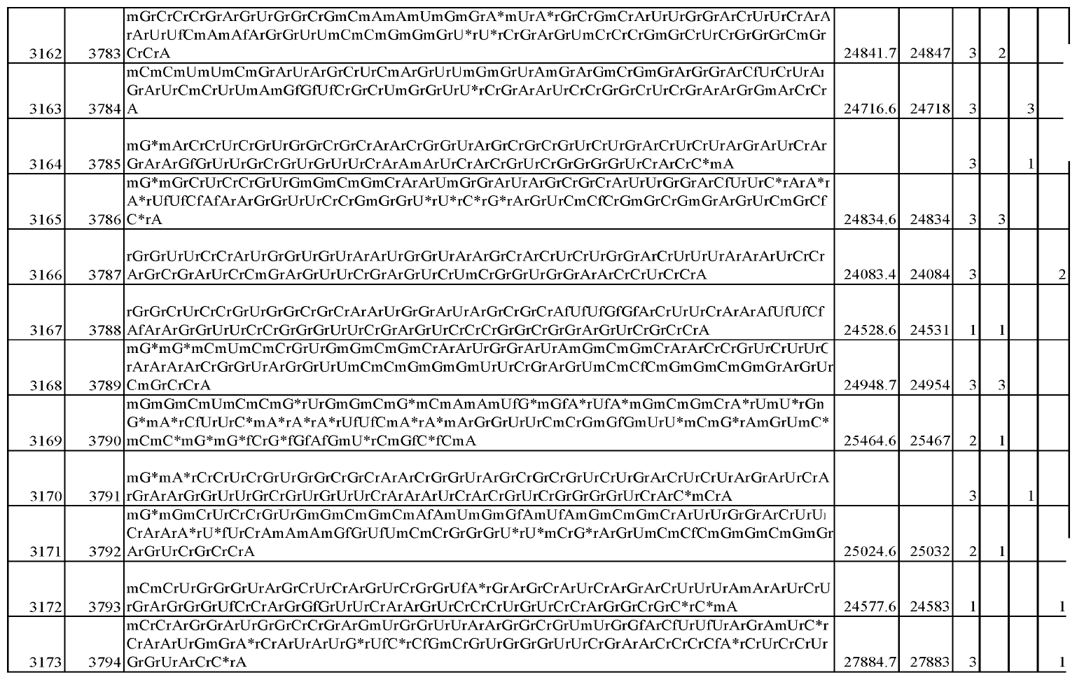

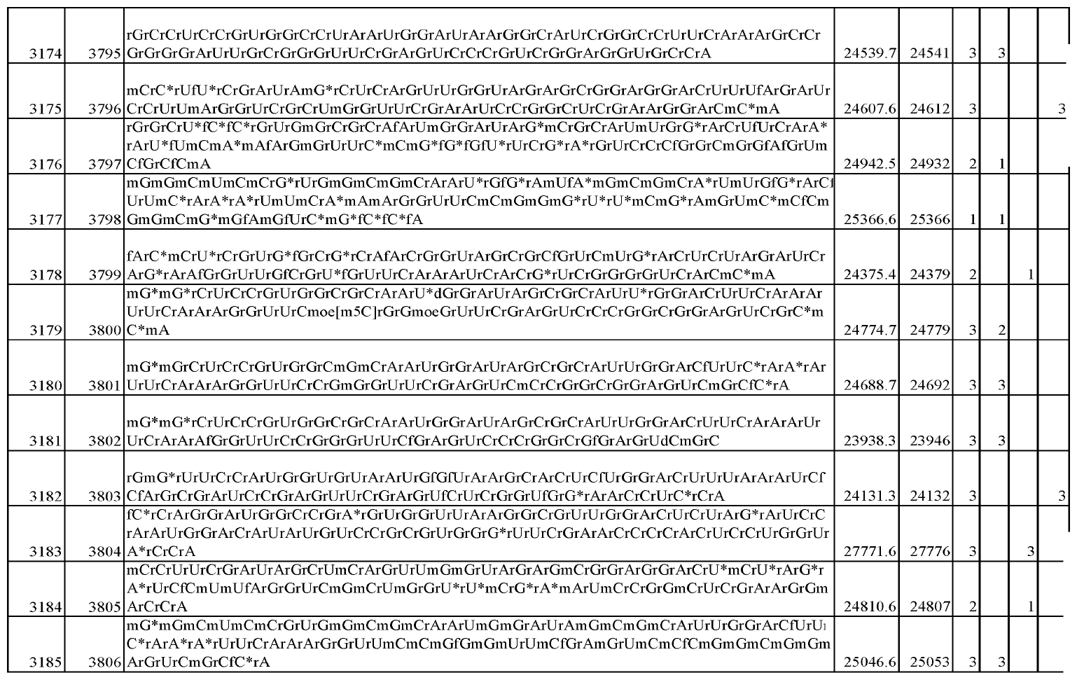

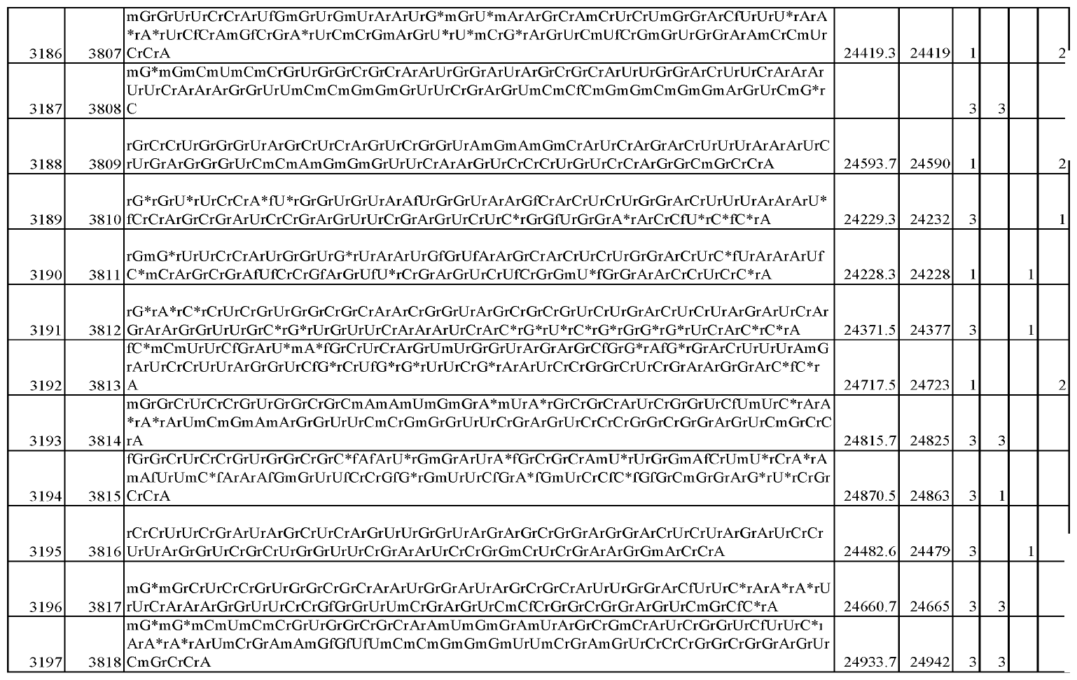

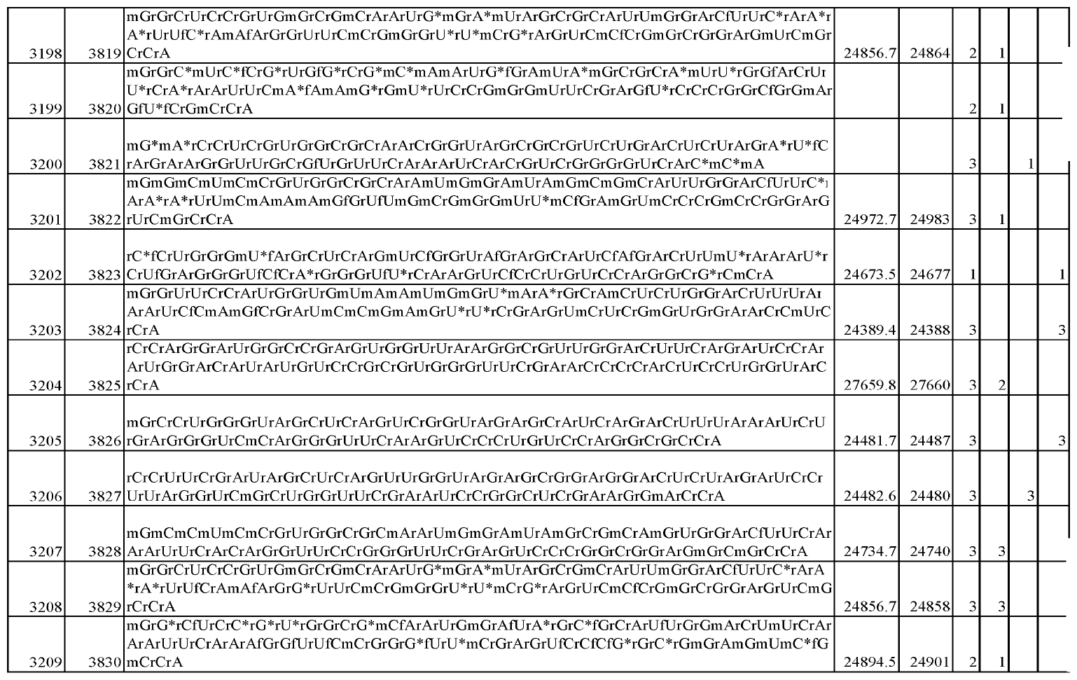

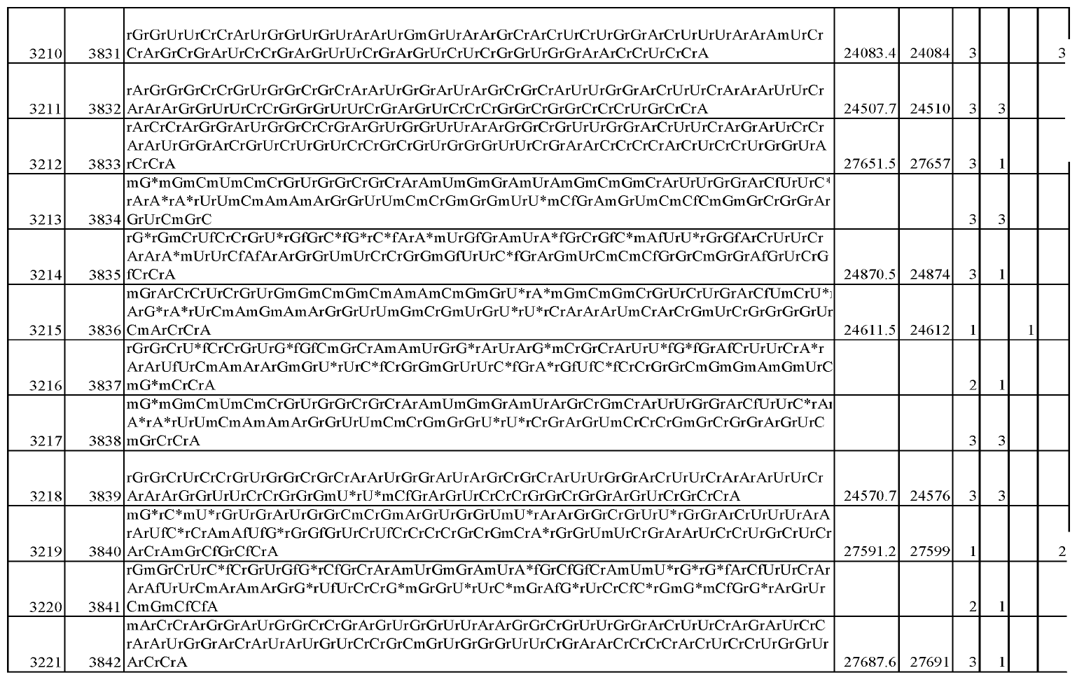

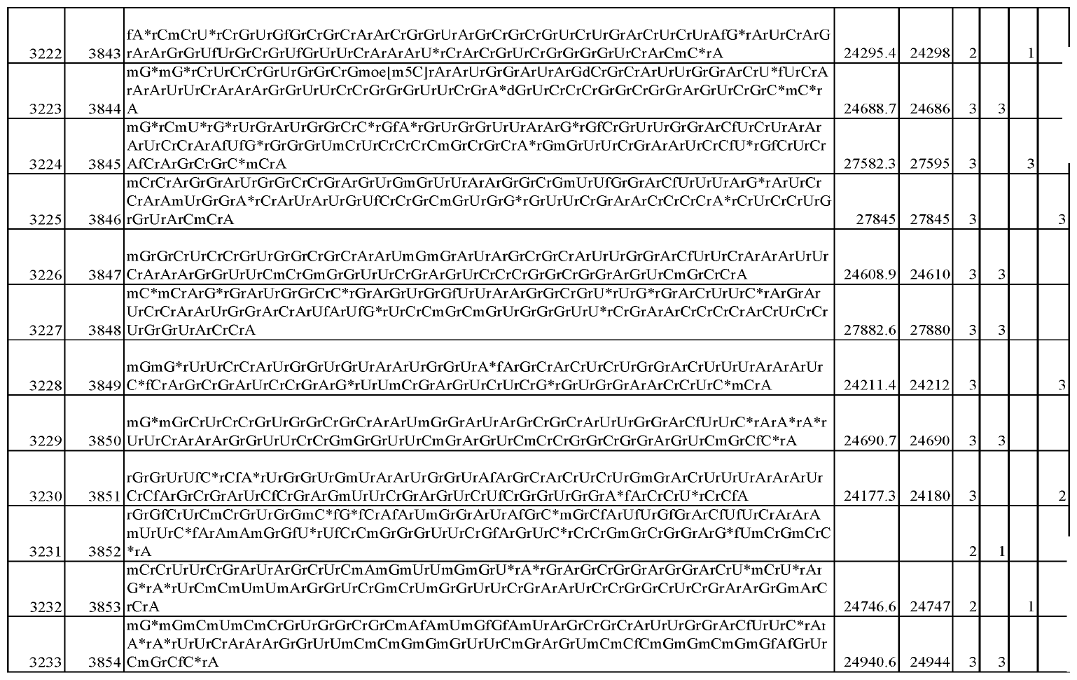

11. The TREM of any one of the preceding embodiments, wherein the TREM has a sequence selected from a sequence provided in FIG. 2.

12. The TREM of any one of the preceding embodiments, wherein the TREM is a TREM provided in FIG. 2.

13. The TREM of any one of the preceding embodiments, wherein the TREM comprises a TREM having at least 70%, 75%, 80%, 85%, 90%, 95%, or 99% sequence identity with a TREM provided in FIG. 2.

14. The TREM of any one of the preceding embodiments, wherein the TREM comprises a sequence that differs by no more than 1, 2, 3, 4, 5, 6, 7, 8, 9, or 10 nucleotides from a TREM provided in FIG. 2.

15. The TREM of any one of the preceding embodiments, wherein the TREM comprises 1, 2, 3, 4, 5, 6, 7, 8, 9, or 10 additional non-naturally occurring modifications compared with a TREM provided in FIG. 2 (e.g., 2′-ribose modifications or an internucleotide modification, e.g., 2′OMe, 2′-halo, 2′-MOE, 2′-deoxy, or phosphorothiorate modifications).

16. The TREM of any one of the preceding embodiments, wherein the TREM is selected from TREM NOs. 1-500, 501-1000, 1001-1500, 1501-2000, 2001-2500, 2501-3000, 3001-3500, 3501-4000, 4001-4500, 4501-5000, 5001-5500, 5501-6000, 6001-6500, 6501-7000, 7001-7500, 7501-8000, 8001-8500, 8501-9000, and 9001-9136 in FIG. 2.

17. The TREM of any one of the preceding embodiments, wherein the TREM has at least 70%, 75%, 80%, 85%, 90%, 95%, or 99% sequence identity to one of TREM NOs. 1-500 in FIG. 2.

18. The TREM of any one of the preceding embodiments, wherein the TREM has at least 70%, 75%, 80%, 85%, 90%, 95%, or 99% sequence identity to one of TREM NOs. 501-1000 in FIG. 2.

19. The TREM of any one of the preceding embodiments, wherein the TREM has at least 70%, 75%, 80%, 85%, 90%, 95%, or 99% sequence identity to one of TREM NOs. 1001-2000 in FIG. 2.

20. The TREM of any one of the preceding embodiments, wherein the TREM has at least 70%, 75%, 80%, 85%, 90%, 95%, or 99% sequence identity to one of TREM NOs. 2001-3000 in FIG. 2.

21. The TREM of any one of the preceding embodiments, wherein the TREM has at least 70%, 75%, 80%, 85%, 90%, 95%, or 99% sequence identity to one of TREM NOs. 3001-4000 in FIG. 2.

22. The TREM of any one of the preceding embodiments, wherein the TREM has at least 70%, 75%, 80%, 85%, 90%, 95%, or 99% sequence identity to one of TREM NOs. 4001-5000 in FIG. 2.

23. The TREM of any one of the preceding embodiments, wherein the TREM has at least 70%, 75%, 80%, 85%, 90%, 95%, or 99% sequence identity to one of TREM NOs. 5001-6000 in FIG. 2.

24. The TREM of any one of the preceding embodiments, wherein the TREM has at least 70%, 75%, 80%, 85%, 90%, 95%, or 99% sequence identity to one of TREM NOs. 6001-7000 in FIG. 2.

25. The TREM of any one of the preceding embodiments, wherein the TREM has at least 70%, 75%, 80%, 85%, 90%, 95%, or 99% sequence identity to one of TREM NOs. 7001-8000 in FIG. 2.

26. The TREM of any one of the preceding embodiments, wherein the TREM has at least 70%, 75%, 80%, 85%, 90%, 95%, or 99% sequence identity to one of TREM NOs. 8001-9000 in FIG. 2.

27. The TREM of any one of the preceding embodiments, wherein the TREM has at least 70%, 75%, 80%, 85%, 90%, 95%, or 99% sequence identity to one of TREM NOs. 9001-9136 in FIG. 2.

28. The TREM of any one of the preceding embodiments, wherein the TREM is selected from TREM NOs. 1-100, 101-200, 201-300, 301-400, 401-500, 501-600, 601-700, 701-800, 801-900, 901-1000, 1001-1100, 1101-1200, 1201-1300, 1301-1400, 1401-1500, 1501-1600, 1601-1700, 1701-1800, 1801-1900, 1901-2000, 2001-2100, 2101-2200, 2201-2300, 2301-2400, 2401-2500, 2501-2600, and 2601-2663 in FIG. 2.

29. The TREM of any one of the preceding embodiments, wherein the TREM is selected from SEQ ID NOs. 1-500, 501-1000, 1001-1500, 1501-2000, 2001-2500, 2501-3000, 3001-3500, 3501-4000, 4001-4500, 4501-5000, 5001-5500, 5501-6000, 6001-6500, 6501-7000, 7001-7500, 7501-8000, 8001-8500, 8501-9000, 9001-9500, and 9501-9757.

30. The TREM of any one of the preceding embodiments, wherein the TREM has at least 70%, 75%, 80%, 85%, 90%, 95%, or 99% sequence identity to one of SEQ ID NOs. 1-500.

31. The TREM of any one of the preceding embodiments, wherein the TREM has at least 70%, 75%, 80%, 85%, 90%, 95%, or 99% sequence identity to one of SEQ ID NOs. 501-1000.

32. The TREM of any one of the preceding embodiments, wherein the TREM has at least 70%, 75%, 80%, 85%, 90%, 95%, or 99% sequence identity to one of SEQ ID NOs. 1001-2000.

33. The TREM of any one of the preceding embodiments, wherein the TREM has at least 70%, 75%, 80%, 85%, 90%, 95%, or 99% sequence identity to one of SEQ ID NOs. 2001-3000.

34. The TREM of any one of the preceding embodiments, wherein the TREM has at least 70%, 75%, 80%, 85%, 90%, 95%, or 99% sequence identity to one of SEQ ID NOs. 3001-4000.

35. The TREM of any one of the preceding embodiments, wherein the TREM has at least 70%, 75%, 80%, 85%, 90%, 95%, or 99% sequence identity to one of SEQ ID NOs. 4001-5000.

36. The TREM of any one of the preceding embodiments, wherein the TREM has at least 70%, 75%, 80%, 85%, 90%, 95%, or 99% sequence identity to one of SEQ ID NOs. 5001-6000.

37. The TREM of any one of the preceding embodiments, wherein the TREM has at least 70%, 75%, 80%, 85%, 90%, 95%, or 99% sequence identity to one of SEQ ID NOs. 6001-7000.

38. The TREM of any one of the preceding embodiments, wherein the TREM has at least 70%, 75%, 80%, 85%, 90%, 95%, or 99% sequence identity to one of SEQ ID NOs. 7001-8000.

39. The TREM of any one of the preceding embodiments, wherein the TREM has at least 70%, 75%, 80%, 85%, 90%, 95%, or 99% sequence identity to one of SEQ ID NOs. 8001-9000.

40. The TREM of any one of the preceding embodiments, wherein the TREM has at least 70%, 75%, 80%, 85%, 90%, 95%, or 99% sequence identity to one of SEQ ID NOs. 9001-9757.

41. The TREM of any one of the preceding embodiments, wherein the TREM is selected from SEQ ID NOs. 1-100, 101-200, 201-300, 301-400, 401-500, 501-600, 601-700, 701-800, 801-900, 901-1000, 1001-1100, 1101-1200, 1201-1300, 1301-1400, 1401-1500, 1501-1600, 1601-1700, 1701-1800, 1801-1900, 1901-2000, 2001-2100, 2101-2200, 2201-2300, 2301-2400, 2401-2500, 2501-2600, 2601-2700, 2701-2800, 2801-2900, 2901-3000, 3001-3100, 3101-3200, and 3201-3284.

42. The TREM of any one of the preceding embodiments, wherein the non-naturally occurring modification is present at a nucleotide position which corresponds to one or more of the nucleotides of SEQ ID NO: 622.

43. The TREM of any one of the preceding embodiments, wherein:
(i) the non-naturally occurring modification is present at a nucleotide position which corresponds to one or more of the nucleotides of SEQ ID NO: 622; and/or
(ii) the TREM differs from the nucleotide sequence of SEQ ID NO: 622 by no more than 1, 2, 3, 4, 5, 6, 7, 8, 9, or 10 nucleotides.

44. The TREM of any one of the preceding embodiments, wherein:
(i) the non-naturally occurring modification is present at a nucleotide position which corresponds to one or more of the nucleotides of SEQ ID NO: 622; and/or
(ii) the TREM comprises the nucleotide sequence of SEQ ID NO: 622.

45. The TREM of any one of the preceding embodiments, wherein the TREM comprises SEQ ID NO: 622 and the non-naturally occurring modification is present at a nucleotide position within in the [ASt Domain1] of SEQ ID NO: 622.

46. The TREM of any one of the preceding embodiments, wherein the TREM comprises SEQ ID NO: 622, the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification, and the non-naturally occurring modification is present at a nucleotide position within in the [ASt Domain1].

47. The TREM of any one of the preceding embodiments, wherein the TREM comprises SEQ ID NO: 622, the non-naturally occurring modification is a 2′-O-methyl (2-OMe) modification, and the non-naturally occurring modification is present at a nucleotide position within in the [ASt Domain1].

48. The TREM of any one of the preceding embodiments, wherein the TREM comprises SEQ ID NO: 622 and does not contain a non-naturally occurring modification within in the [ASt Domain1].

49. The TREM of any one of the preceding embodiments, wherein the TREM comprises SEQ ID NO: 622 and the non-naturally occurring modification is present at a nucleotide position within in the [DH Domain] of SEQ ID NO: 622.

50. The TREM of any one of the preceding embodiments, wherein the TREM comprises SEQ ID NO: 622, the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification, and the non-naturally occurring modification is present at a nucleotide position within in the [DH Domain].

51. The TREM of any one of the preceding embodiments, wherein the TREM comprises SEQ ID NO: 622, the non-naturally occurring modification is a 2′-O-methyl (2-OMe) or 2′-halo (e.g., 2′F or 2′Cl) modification, and the non-naturally occurring modification is present at a nucleotide position within in the [DH Domain].

52. The TREM of any one of the preceding embodiments, wherein the TREM comprises SEQ ID NO: 622 and does not contain a non-naturally occurring modification within in the [DH Domain].

53. The TREM of any one of the preceding embodiments, wherein the TREM comprises SEQ ID NO: 622 and the non-naturally occurring modification is present at a nucleotide position within in the [ACH Domain] of SEQ ID NO: 622.

54. The TREM of any one of the preceding embodiments, wherein the TREM comprises SEQ ID NO: 622, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [ACH Domain].

55. The TREM of any one of the preceding embodiments, wherein the TREM comprises SEQ ID NO: 622, the non-naturally occurring modification is a 2′-O-methyl (2-OMe) or 2′-halo (e.g., 2′F or 2′Cl) modification, and the non-naturally occurring modification is present at a nucleotide position within in the [ACH Domain].

56. The TREM of any one of the preceding embodiments, wherein the TREM comprises SEQ ID NO: 622 and does not contain a non-naturally occurring modification within in the [ACH Domain].

57. The TREM of any one of the preceding embodiments, wherein the TREM comprises SEQ ID NO: 622 and the non-naturally occurring modification is present at a nucleotide position within in the [VL Domain] of SEQ ID NO: 622.

58. The TREM of any one of the preceding embodiments, wherein the TREM comprises SEQ ID NO: 622, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [VL Domain].

59. The TREM of any one of the preceding embodiments, wherein the TREM comprises SEQ ID NO: 622, the non-naturally occurring modification is a 2′-O-methyl (2-OMe) or 2′-halo (e.g., 2′F or 2′Cl) modification, and the non-naturally occurring modification is present at a nucleotide position within in the [VL Domain].

60. The TREM of any one of the preceding embodiments, wherein the TREM comprises SEQ ID NO: 622 and does not contain a non-naturally occurring modification within in the [VL Domain].

61. The TREM of any one of the preceding embodiments, wherein the TREM comprises SEQ ID NO: 622 and the non-naturally occurring modification is present at a nucleotide position within in the [TH Domain] of SEQ ID NO: 622.

62. The TREM of any one of the preceding embodiments, wherein the TREM comprises SEQ ID NO: 622, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [TH Domain].

63. The TREM of any one of the preceding embodiments, wherein the TREM comprises SEQ ID NO: 622, the non-naturally occurring modification is a 2′-O-methyl (2-OMe) or 2′-halo (e.g., 2′F or 2′Cl) modification, and the non-naturally occurring modification is present at a nucleotide position within in the [TH Domain].

64. The TREM of any one of the preceding embodiments, wherein the TREM comprises SEQ ID NO: 622 and does not contain a non-naturally occurring modification within in the [TH Domain].

65. The TREM of any one of the preceding embodiments, wherein the TREM comprises SEQ ID NO: 622 and the non-naturally occurring modification is present at a nucleotide position within in the [ASt Domain2] of SEQ ID NO: 622.

66. The TREM of any one of the preceding embodiments, wherein the TREM comprises SEQ ID NO: 622, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [ASt Domain2].

67. The TREM of any one of the preceding embodiments, wherein the TREM comprises SEQ ID NO: 622, the non-naturally occurring modification is a 2′-O-methyl (2-OMe) or 2′-halo (e.g., 2′F or 2′Cl) modification, and the non-naturally occurring modification is present at a nucleotide position within in the [ASt Domain2].

68. The TREM of any one of the preceding embodiments, wherein the TREM comprises SEQ ID NO: 622 and does not contain a non-naturally occurring modification within in the [ASt Domain2].

69. The TREM of any one of the preceding embodiments, wherein the non-naturally occurring modification is present at a nucleotide position which corresponds to one or more of the nucleotides of SEQ ID NO: 623.

70. The TREM of any one of the preceding embodiments, wherein:
(i) the non-naturally occurring modification is present at a nucleotide position which corresponds to one or more of the nucleotides of SEQ ID NO: 623; and/or
(ii) the TREM differs from the nucleotide sequence of SEQ ID NO: 623 by no more than 1, 2, 3, 4, 5, 6, 7, 8, 9, or 10 nucleotides.

71. The TREM of any one of the preceding embodiments, wherein:
(i) the non-naturally occurring modification is present at a nucleotide position which corresponds to one or more of the nucleotides of SEQ ID NO: 623; and/or
(ii) the TREM comprises the nucleotide sequence of SEQ ID NO: 623.

72. The TREM of any one of the preceding embodiments, wherein the TREM comprises SEQ ID NO: 623 and the non-naturally occurring modification is present at a nucleotide position within in the [ASt Domain1] of SEQ ID NO: 623.

73. The TREM of any one of the preceding embodiments, wherein the TREM comprises SEQ ID NO: 623, the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification, and the non-naturally occurring modification is present at a nucleotide position within in the [ASt Domain1].

74. The TREM of any one of the preceding embodiments, wherein the TREM comprises SEQ ID NO: 623, the non-naturally occurring modification is a 2′-O-methyl (2-OMe) modification, and the non-naturally occurring modification is present at a nucleotide position within in the [ASt Domain1].

75. The TREM of any one of the preceding embodiments, wherein the TREM comprises SEQ ID NO: 623 and does not contain a non-naturally occurring modification within in the [ASt Domain1].

76. The TREM of any one of the preceding embodiments, wherein the TREM comprises SEQ ID NO: 623 and the non-naturally occurring modification is present at a nucleotide position within in the [DH Domain] of SEQ ID NO: 623.

77. The TREM of any one of the preceding embodiments, wherein the TREM comprises SEQ ID NO: 623, the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification, and the non-naturally occurring modification is present at a nucleotide position within in the [DH Domain].

78. The TREM of any one of the preceding embodiments, wherein the TREM comprises SEQ ID NO: 623, the non-naturally occurring modification is a 2′-O-methyl (2-OMe) or 2′-halo (e.g., 2′F or 2′Cl) modification, and the non-naturally occurring modification is present at a nucleotide position within in the [DH Domain].

79. The TREM of any one of the preceding embodiments, wherein the TREM comprises SEQ ID NO: 623 and does not contain a non-naturally occurring modification within in the [DH Domain].

80. The TREM of any one of the preceding embodiments, wherein the TREM comprises SEQ ID NO: 623 and the non-naturally occurring modification is present at a nucleotide position within in the [ACH Domain] of SEQ ID NO: 623.

81. The TREM of any one of the preceding embodiments, wherein the TREM comprises SEQ ID NO: 623, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [ACH Domain].

82. The TREM of any one of the preceding embodiments, wherein the TREM comprises SEQ ID NO: 623, the non-naturally occurring modification is a 2′-O-methyl (2-OMe) or 2′-halo (e.g., 2′F or 2′Cl) modification, and the non-naturally occurring modification is present at a nucleotide position within in the [ACH Domain].

83. The TREM of any one of the preceding embodiments, wherein the TREM comprises SEQ ID NO: 623 and does not contain a non-naturally occurring modification within in the [ACH Domain].

84. The TREM of any one of the preceding embodiments, wherein the TREM comprises SEQ ID NO: 623 and the non-naturally occurring modification is present at a nucleotide position within in the [VL Domain] of SEQ ID NO: 623.

85. The TREM of any one of the preceding embodiments, wherein the TREM comprises SEQ ID NO: 623, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [VL Domain].

86. The TREM of any one of the preceding embodiments, wherein the TREM comprises SEQ ID NO: 623, the non-naturally occurring modification is a 2′-O-methyl (2-OMe) or 2′-halo (e.g., 2′F or 2′Cl) modification, and the non-naturally occurring modification is present at a nucleotide position within in the [VL Domain].

87. The TREM of any one of the preceding embodiments, wherein the TREM comprises SEQ ID NO: 623 and does not contain a non-naturally occurring modification within in the [VL Domain].

88. The TREM of any one of the preceding embodiments, wherein the TREM comprises SEQ ID NO: 623 and the non-naturally occurring modification is present at a nucleotide position within in the [TH Domain] of SEQ ID NO: 623.

89. The TREM of any one of the preceding embodiments, wherein the TREM comprises SEQ ID NO: 623, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [TH Domain].

90. The TREM of any one of the preceding embodiments, wherein the TREM comprises SEQ ID NO: 623, the non-naturally occurring modification is a 2′-O-methyl (2-OMe) or 2′-halo (e.g., 2′F or 2′Cl) modification, and the non-naturally occurring modification is present at a nucleotide position within in the [TH Domain].

91. The TREM of any one of the preceding embodiments, wherein the TREM comprises SEQ ID NO: 623 and does not contain a non-naturally occurring modification within in the [TH Domain].

92. The TREM of any one of the preceding embodiments, wherein the TREM comprises SEQ ID NO: 623 and the non-naturally occurring modification is present at a nucleotide position within in the [ASt Domain2] of SEQ ID NO: 623.

93. The TREM of any one of the preceding embodiments, wherein the TREM comprises SEQ ID NO: 623, the non-naturally occurring modification is selected from a 2′-O-methyl (2-modification, and the non-naturally occurring modification is present at a nucleotide position within in the [ASt Domain2].

94. The TREM of any one of the preceding embodiments, wherein the TREM comprises SEQ ID NO: 623, the non-naturally occurring modification is a 2′-O-methyl (2-OMe) or 2′-halo (e.g., 2′F or 2′Cl) modification, and the non-naturally occurring modification is present at a nucleotide position within in the [ASt Domain2].

95. The TREM of any one of the preceding embodiments, wherein the TREM comprises SEQ ID NO: 623 and does not contain a non-naturally occurring modification within in the [ASt Domain2].

96. The TREM of any one of the preceding embodiments, wherein the non-naturally occurring modification is present at a nucleotide position which corresponds to one or more of the nucleotides of SEQ ID NO: 624.

97. The TREM of any one of the preceding embodiments, wherein:
(i) the non-naturally occurring modification is present at a nucleotide position which corresponds to one or more of the nucleotides of SEQ ID NO: 624; and/or
(ii) the TREM differs from the nucleotide sequence of SEQ ID NO: 624 by no more than 1, 2, 3, 4, 5, 6, 7, 8, 9, or 10 nucleotides.

98. The TREM of any one of the preceding embodiments, wherein:
(i) the non-naturally occurring modification is present at a nucleotide position which corresponds to one or more of the nucleotides of SEQ ID NO: 624; and/or
(ii) the TREM comprises the nucleotide sequence of SEQ ID NO: 624.

99. The TREM of any one of the preceding embodiments, wherein the TREM comprises SEQ ID NO: 624 and the non-naturally occurring modification is present at a nucleotide position within in the [ASt Domain1] of SEQ ID NO: 624.

100. The TREM of any one of the preceding embodiments, wherein the TREM comprises SEQ ID NO: 624, the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification, and the non-naturally occurring modification is present at a nucleotide position within in the [ASt Domain1].

101. The TREM of any one of the preceding embodiments, wherein the TREM comprises SEQ ID NO: 624, the non-naturally occurring modification is a 2′-O-methyl (2-OMe) modification, and the non-naturally occurring modification is present at a nucleotide position within in the [ASt Domain1].

102. The TREM of any one of the preceding embodiments, wherein the TREM comprises SEQ ID NO: 624 and does not contain a non-naturally occurring modification within in the [ASt Domain1].

103. The TREM of any one of the preceding embodiments, wherein the TREM comprises SEQ ID NO: 624 and the non-naturally occurring modification is present at a nucleotide position within in the [DH Domain] of SEQ ID NO: 624.

104. The TREM of any one of the preceding embodiments, wherein the TREM comprises SEQ ID NO: 624, the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification, and the non-naturally occurring modification is present at a nucleotide position within in the [DH Domain].

105. The TREM of any one of the preceding embodiments, wherein the TREM comprises SEQ ID NO: 624, the non-naturally occurring modification is a 2′-O-methyl (2-OMe) or 2′-halo (e.g., 2′F or 2′Cl) modification, and the non-naturally occurring modification is present at a nucleotide position within in the [DH Domain].

106. The TREM of any one of the preceding embodiments, wherein the TREM comprises SEQ ID NO: 624 and does not contain a non-naturally occurring modification within in the [DH Domain].

107. The TREM of any one of the preceding embodiments, wherein the TREM comprises SEQ ID NO: 624 and the non-naturally occurring modification is present at a nucleotide position within in the [ACH Domain] of SEQ ID NO: 624.

108. The TREM of any one of the preceding embodiments, wherein the TREM comprises SEQ ID NO: 624, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [ACH Domain].

109. The TREM of any one of the preceding embodiments, wherein the TREM comprises SEQ ID NO: 624, the non-naturally occurring modification is a 2′-O-methyl (2-OMe) or 2′-halo (e.g., 2′F or 2′Cl) modification, and the non-naturally occurring modification is present at a nucleotide position within in the [ACH Domain].

110. The TREM of any one of the preceding embodiments, wherein the TREM comprises SEQ ID NO: 624 and does not contain a non-naturally occurring modification within in the [ACH Domain].

111. The TREM of any one of the preceding embodiments, wherein the TREM comprises SEQ ID NO: 624 and the non-naturally occurring modification is present at a nucleotide position within in the [VL Domain] of SEQ ID NO: 624.

112. The TREM of any one of the preceding embodiments, wherein the TREM comprises SEQ ID NO: 624, the non-naturally occurring modification is selected from a 2′-O-methyl (2-modification, and the non-naturally occurring modification is present at a nucleotide position within in the [VL Domain].

113. The TREM of any one of the preceding embodiments, wherein the TREM comprises SEQ ID NO: 624, the non-naturally occurring modification is a 2′-O-methyl (2-OMe) or 2′-halo (e.g., 2′F or 2′Cl) modification, and the non-naturally occurring modification is present at a nucleotide position within in the [VL Domain].

114. The TREM of any one of the preceding embodiments, wherein the TREM comprises SEQ ID NO: 624 and does not contain a non-naturally occurring modification within in the [VL Domain].

115. The TREM of any one of the preceding embodiments, wherein the TREM comprises SEQ ID NO: 624 and the non-naturally occurring modification is present at a nucleotide position within in the [TH Domain] of SEQ ID NO: 624.

116. The TREM of any one of the preceding embodiments, wherein the TREM comprises SEQ ID NO: 624, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [TH Domain].

117. The TREM of any one of the preceding embodiments, wherein the TREM comprises SEQ ID NO: 624, the non-naturally occurring modification is a 2′-O-methyl (2-OMe) or 2′-halo (e.g., 2′F or 2′Cl) modification, and the non-naturally occurring modification is present at a nucleotide position within in the [TH Domain].

118. The TREM of any one of the preceding embodiments, wherein the TREM comprises SEQ ID NO: 624 and does not contain a non-naturally occurring modification within in the [TH Domain].

119. The TREM of any one of the preceding embodiments, wherein the TREM comprises SEQ ID NO: 624 and the non-naturally occurring modification is present at a nucleotide position within in the [ASt Domain2] of SEQ ID NO: 624.

120. The TREM of any one of the preceding embodiments, wherein the TREM comprises SEQ ID NO: 624, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [ASt Domain2].

121. The TREM of any one of the preceding embodiments, wherein the TREM comprises SEQ ID NO: 624, the non-naturally occurring modification is a 2′-O-methyl (2-OMe) or 2′-halo (e.g., 2′F or 2′Cl) modification, and the non-naturally occurring modification is present at a nucleotide position within in the [ASt Domain2].

122. The TREM of any one of the preceding embodiments, wherein the TREM comprises SEQ ID NO: 624 and does not contain a non-naturally occurring modification within in the [ASt Domain2].

123. The TREM of any one of the preceding embodiments, wherein the non-naturally occurring modification is present at a nucleotide position which corresponds to one or more of the nucleotides of SEQ ID NO: 6967.

124. The TREM of any one of the preceding embodiments, wherein:
(i) the non-naturally occurring modification is present at a nucleotide position which corresponds to one or more of the nucleotides of SEQ ID NO: 6967; and/or
(ii) the TREM differs from the nucleotide sequence of SEQ ID NO: 6967 by no more than 1, 2, 3, 4, 5, 6, 7, 8, 9, or 10 nucleotides.

125. The TREM of any one of the preceding embodiments, wherein:
(i) the non-naturally occurring modification is present at a nucleotide position which corresponds to one or more of the nucleotides of SEQ ID NO: 6967; and/or
(ii) the TREM comprises the nucleotide sequence of SEQ ID NO: 6967.

126. The TREM of any one of the preceding embodiments, wherein the TREM comprises SEQ ID NO: 6967 and the non-naturally occurring modification is present at a nucleotide position within in the [ASt Domain1] of SEQ ID NO: 6967.

127. The TREM of any one of the preceding embodiments, wherein the TREM comprises SEQ ID NO: 6967, the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification, and the non-naturally occurring modification is present at a nucleotide position within in the [ASt Domain1].

128. The TREM of any one of the preceding embodiments, wherein the TREM comprises SEQ ID NO: 6967, the non-naturally occurring modification is a 2′-O-methyl (2-OMe) modification, and the non-naturally occurring modification is present at a nucleotide position within in the [ASt Domain1].

129. The TREM of any one of the preceding embodiments, wherein the TREM comprises SEQ ID NO: 6967 and does not contain a non-naturally occurring modification within in the [ASt Domain1].

130. The TREM of any one of the preceding embodiments, wherein the TREM comprises SEQ ID NO: 6967 and the non-naturally occurring modification is present at a nucleotide position within in the [DH Domain] of SEQ ID NO: 6967.

131. The TREM of any one of the preceding embodiments, wherein the TREM comprises SEQ ID NO: 6967, the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification, and the non-naturally occurring modification is present at a nucleotide position within in the [DH Domain].

132. The TREM of any one of the preceding embodiments, wherein the TREM comprises SEQ ID NO: 6967, the non-naturally occurring modification is a 2′-O-methyl (2-OMe) or 2′-halo (e.g., 2′F or 2′Cl) modification, and the non-naturally occurring modification is present at a nucleotide position within in the [DH Domain].

133. The TREM of any one of the preceding embodiments, wherein the TREM comprises SEQ ID NO: 6967 and does not contain a non-naturally occurring modification within in the [DH Domain].

134. The TREM of any one of the preceding embodiments, wherein the TREM comprises SEQ ID NO: 6967 and the non-naturally occurring modification is present at a nucleotide position within in the [ACH Domain] of SEQ ID NO: 6967.

135. The TREM of any one of the preceding embodiments, wherein the TREM comprises SEQ ID NO: 6967, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [ACH Domain].

136. The TREM of any one of the preceding embodiments, wherein the TREM comprises SEQ ID NO: 6967, the non-naturally occurring modification is a 2′-O-methyl (2-OMe) or 2′-halo (e.g., 2′F or 2′Cl) modification, and the non-naturally occurring modification is present at a nucleotide position within in the [ACH Domain].

137. The TREM of any one of the preceding embodiments, wherein the TREM comprises SEQ ID NO: 6967 and does not contain a non-naturally occurring modification within in the [ACH Domain].

138. The TREM of any one of the preceding embodiments, wherein the TREM comprises SEQ ID NO: 6967 and the non-naturally occurring modification is present at a nucleotide position within in the [VL Domain] of SEQ ID NO: 6967.

139. The TREM of any one of the preceding embodiments, wherein the TREM comprises SEQ ID NO: 6967, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [VL Domain].

140. The TREM of any one of the preceding embodiments, wherein the TREM comprises SEQ ID NO: 6967, the non-naturally occurring modification is a 2′-O-methyl (2-OMe) or 2′-halo (e.g., 2′F or 2′Cl) modification, and the non-naturally occurring modification is present at a nucleotide position within in the [VL Domain].

141. The TREM of any one of the preceding embodiments, wherein the TREM comprises SEQ ID NO: 6967 and does not contain a non-naturally occurring modification within in the [VL Domain].

142. The TREM of any one of the preceding embodiments, wherein the TREM comprises SEQ ID NO: 6967 and the non-naturally occurring modification is present at a nucleotide position within in the [TH Domain] of SEQ ID NO: 6967.

143. The TREM of any one of the preceding embodiments, wherein the TREM comprises SEQ ID NO: 6967, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [TH Domain].

144. The TREM of any one of the preceding embodiments, wherein the TREM comprises SEQ ID NO: 6967, the non-naturally occurring modification is a 2′-O-methyl (2-OMe) or 2′-halo (e.g., 2′F or 2′Cl) modification, and the non-naturally occurring modification is present at a nucleotide position within in the [TH Domain].

145. The TREM of any one of the preceding embodiments, wherein the TREM comprises SEQ ID NO: 6967 and does not contain a non-naturally occurring modification within in the [TH Domain].

146. The TREM of any one of the preceding embodiments, wherein the TREM comprises SEQ ID NO: 6967 and the non-naturally occurring modification is present at a nucleotide position within in the [ASt Domain2] of SEQ ID NO: 6967.

147. The TREM of any one of the preceding embodiments, wherein the TREM comprises SEQ ID NO: 6967, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [ASt Domain2].

148. The TREM of any one of the preceding embodiments, wherein the TREM comprises SEQ ID NO: 6967, the non-naturally occurring modification is a 2′-O-methyl (2-OMe) or 2′-halo (e.g., 2′F or 2′Cl) modification, and the non-naturally occurring modification is present at a nucleotide position within in the [ASt Domain2].

149. The TREM of any one of the preceding embodiments, wherein the TREM comprises SEQ ID NO: 6967 and does not contain a non-naturally occurring modification within in the [ASt Domain2].

150. The TREM of any one of the preceding embodiments, wherein the non-naturally occurring modification is present at a nucleotide position which corresponds to one or more of the nucleotides of SEQ ID NO: 4386.

151. The TREM of any one of the preceding embodiments, wherein:
(i) the non-naturally occurring modification is present at a nucleotide position which corresponds to one or more of the nucleotides of SEQ ID NO: 4386; and/or
(ii) the TREM differs from the nucleotide sequence of SEQ ID NO: 4386 by no more than 1, 2, 3, 4, 5, 6, 7, 8, 9, or 10 nucleotides.

152. The TREM of any one of the preceding embodiments, wherein:
(i) the non-naturally occurring modification is present at a nucleotide position which corresponds to one or more of the nucleotides of SEQ ID NO: 4386; and/or
(ii) the TREM comprises the nucleotide sequence of SEQ ID NO: 4386.

153. The TREM of any one of the preceding embodiments, wherein the TREM comprises SEQ ID NO: 4386 and the non-naturally occurring modification is present at a nucleotide position within in the [ASt Domain1] of SEQ ID NO: 4386.

154. The TREM of any one of the preceding embodiments, wherein the TREM comprises SEQ ID NO: 4386, the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification, and the non-naturally occurring modification is present at a nucleotide position within in the [ASt Domain1].

155. The TREM of any one of the preceding embodiments, wherein the TREM comprises SEQ ID NO: 4386, the non-naturally occurring modification is a 2′-O-methyl (2-OMe) modification, and the non-naturally occurring modification is present at a nucleotide position within in the [ASt Domain1].

166. The TREM of any one of the preceding embodiments, wherein the TREM comprises SEQ ID NO: 4386 and does not contain a non-naturally occurring modification within in the [ASt Domain1].

167. The TREM of any one of the preceding embodiments, wherein the TREM comprises SEQ ID NO: 4386 and the non-naturally occurring modification is present at a nucleotide position within in the [DH Domain] of SEQ ID NO: 4386.

168. The TREM of any one of the preceding embodiments, wherein the TREM comprises SEQ ID NO: 4386, the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification, and the non-naturally occurring modification is present at a nucleotide position within in the [DH Domain].

169. The TREM of any one of the preceding embodiments, wherein the TREM comprises SEQ ID NO: 4386, the non-naturally occurring modification is a 2′-O-methyl (2-OMe) or 2′-halo (e.g., 2′F or 2′Cl) modification, and the non-naturally occurring modification is present at a nucleotide position within in the [DH Domain].

170. The TREM of any one of the preceding embodiments, wherein the TREM comprises SEQ ID NO: 4386 and does not contain a non-naturally occurring modification within in the [DH Domain].

171. The TREM of any one of the preceding embodiments, wherein the TREM comprises SEQ ID NO: 4386 and the non-naturally occurring modification is present at a nucleotide position within in the [ACH Domain] of SEQ ID NO: 4386.

172. The TREM of any one of the preceding embodiments, wherein the TREM comprises SEQ ID NO: 4386, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [ACH Domain].

173. The TREM of any one of the preceding embodiments, wherein the TREM comprises SEQ ID NO: 4386, the non-naturally occurring modification is a 2′-O-methyl (2-OMe) or 2′-halo (e.g., 2′F or 2′Cl) modification, and the non-naturally occurring modification is present at a nucleotide position within in the [ACH Domain].

174. The TREM of any one of the preceding embodiments, wherein the TREM comprises SEQ ID NO: 4386 and does not contain a non-naturally occurring modification within in the [ACH Domain].

175. The TREM of any one of the preceding embodiments, wherein the TREM comprises SEQ ID NO: 4386 and the non-naturally occurring modification is present at a nucleotide position within in the [VL Domain] of SEQ ID NO: 4386.

176. The TREM of any one of the preceding embodiments, wherein the TREM comprises SEQ ID NO: 4386, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [VL Domain].

177. The TREM of any one of the preceding embodiments, wherein the TREM comprises SEQ ID NO: 4386, the non-naturally occurring modification is a 2′-O-methyl (2-OMe) or 2′-halo (e.g., 2′F or 2′Cl) modification, and the non-naturally occurring modification is present at a nucleotide position within in the [VL Domain].

178. The TREM of any one of the preceding embodiments, wherein the TREM comprises SEQ ID NO: 4386 and does not contain a non-naturally occurring modification within in the [VL Domain].

179. The TREM of any one of the preceding embodiments, wherein the TREM comprises SEQ ID NO: 4386 and the non-naturally occurring modification is present at a nucleotide position within in the [TH Domain] of SEQ ID NO: 4386.

180. The TREM of any one of the preceding embodiments, wherein the TREM comprises SEQ ID NO: 4386, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [TH Domain].

181. The TREM of any one of the preceding embodiments, wherein the TREM comprises SEQ ID NO: 4386, the non-naturally occurring modification is a 2′-O-methyl (2-OMe) or 2′-halo (e.g., 2′F or 2′Cl) modification, and the non-naturally occurring modification is present at a nucleotide position within in the [TH Domain].

182. The TREM of any one of the preceding embodiments, wherein the TREM comprises SEQ ID NO: 4386 and does not contain a non-naturally occurring modification within in the [TH Domain].

183. The TREM of any one of the preceding embodiments, wherein the TREM comprises SEQ ID NO: 4386 and the non-naturally occurring modification is present at a nucleotide position within in the [ASt Domain2] of SEQ ID NO: 4386.

184. The TREM of any one of the preceding embodiments, wherein the TREM comprises SEQ ID NO: 4386, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [ASt Domain2].

185. The TREM of any one of the preceding embodiments, wherein the TREM comprises SEQ ID NO: 4386, the non-naturally occurring modification is a 2′-O-methyl (2-OMe) or 2′-halo (e.g., 2′F or 2′Cl) modification, and the non-naturally occurring modification is present at a nucleotide position within in the [ASt Domain].

186. The TREM of any one of the preceding embodiments, wherein the TREM comprises SEQ ID NO: 4386 and does not contain a non-naturally occurring modification within in the [ASt Domain2].

187. The TREM of any one of the preceding embodiments, wherein the non-naturally occurring modification is present at a nucleotide position which corresponds to one or more of the nucleotides of SEQ ID NO: 4834.

188. The TREM of any one of the preceding embodiments, wherein:
(i) the non-naturally occurring modification is present at a nucleotide position which corresponds to one or more of the nucleotides of SEQ ID NO: 4834; and/or
(ii) the TREM differs from the nucleotide sequence of SEQ ID NO: 4834 by no more than 1, 2, 3, 4, 5, 6, 7, 8, 9, or 10 nucleotides.

189. The TREM of any one of the preceding embodiments, wherein:
(i) the non-naturally occurring modification is present at a nucleotide position which corresponds to one or more of the nucleotides of SEQ ID NO: 4386; and/or
(ii) the TREM comprises the nucleotide sequence of SEQ ID NO: 4386.

190. The TREM of any one of the preceding embodiments, wherein the TREM comprises SEQ ID NO: 4834 and the non-naturally occurring modification is present at a nucleotide position within in the [ASt Domain1] of SEQ ID NO: 4834.

191. The TREM of any one of the preceding embodiments, wherein the TREM comprises SEQ ID NO: 4834, the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification, and the non-naturally occurring modification is present at a nucleotide position within in the [ASt Domain1].

192. The TREM of any one of the preceding embodiments, wherein the TREM comprises SEQ ID NO: 4834, the non-naturally occurring modification is a 2′-O-methyl (2-OMe) modification, and the non-naturally occurring modification is present at a nucleotide position within in the [ASt Domain1].

193. The TREM of any one of the preceding embodiments, wherein the TREM comprises SEQ ID NO: 4834 and does not contain a non-naturally occurring modification within in the [ASt Domain1].

194. The TREM of any one of the preceding embodiments, wherein the TREM comprises SEQ ID NO: 4834 and the non-naturally occurring modification is present at a nucleotide position within in the [DH Domain] of SEQ ID NO: 4834.

195. The TREM of any one of the preceding embodiments, wherein the TREM comprises SEQ ID NO: 4834, the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification, and the non-naturally occurring modification is present at a nucleotide position within in the [DH Domain].

196. The TREM of any one of the preceding embodiments, wherein the TREM comprises SEQ ID NO: 4834, the non-naturally occurring modification is a 2′-O-methyl (2-OMe) or 2′-halo (e.g., 2′F or 2′Cl) modification, and the non-naturally occurring modification is present at a nucleotide position within in the [DH Domain].

197. The TREM of any one of the preceding embodiments, wherein the TREM comprises SEQ ID NO: 4834 and does not contain a non-naturally occurring modification within in the [DH Domain].

198. The TREM of any one of the preceding embodiments, wherein the TREM comprises SEQ ID NO: 4834 and the non-naturally occurring modification is present at a nucleotide position within in the [ACH Domain] of SEQ ID NO: 4834.

199. The TREM of any one of the preceding embodiments, wherein the TREM comprises SEQ ID NO: 4834, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [ACH Domain].

200. The TREM of any one of the preceding embodiments, wherein the TREM comprises SEQ ID NO: 4834, the non-naturally occurring modification is a 2′-O-methyl (2-OMe) or 2′-halo (e.g., 2′F or 2′Cl) modification, and the non-naturally occurring modification is present at a nucleotide position within in the [ACH Domain].

201. The TREM of any one of the preceding embodiments, wherein the TREM comprises SEQ ID NO: 4834 and does not contain a non-naturally occurring modification within in the [ACH Domain].

202. The TREM of any one of the preceding embodiments, wherein the TREM comprises SEQ ID NO: 4834 and the non-naturally occurring modification is present at a nucleotide position within in the [VL Domain] of SEQ ID NO: 4834.

203. The TREM of any one of the preceding embodiments, wherein the TREM comprises SEQ ID NO: 4834, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [VL Domain].

204. The TREM of any one of the preceding embodiments, wherein the TREM comprises SEQ ID NO: 4834, the non-naturally occurring modification is a 2′-O-methyl (2-OMe) or 2′-halo (e.g., 2′F or 2′Cl) modification, and the non-naturally occurring modification is present at a nucleotide position within in the [VL Domain].

205. The TREM of any one of the preceding embodiments, wherein the TREM comprises SEQ ID NO: 4834 and does not contain a non-naturally occurring modification within in the [VL Domain].

206. The TREM of any one of the preceding embodiments, wherein the TREM comprises SEQ ID NO: 4834 and the non-naturally occurring modification is present at a nucleotide position within in the [TH Domain] of SEQ ID NO: 4834.

207. The TREM of any one of the preceding embodiments, wherein the TREM comprises SEQ ID NO: 4386, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [TH Domain].

208. The TREM of any one of the preceding embodiments, wherein the TREM comprises SEQ ID NO: 4834, the non-naturally occurring modification is a 2′-O-methyl (2-OMe) or 2′-halo (e.g., 2′F or 2′Cl) modification, and the non-naturally occurring modification is present at a nucleotide position within in the [TH Domain].

209. The TREM of any one of the preceding embodiments, wherein the TREM comprises SEQ ID NO: 4834 and does not contain a non-naturally occurring modification within in the [TH Domain].

210. The TREM of any one of the preceding embodiments, wherein the TREM comprises SEQ ID NO: 4834 and the non-naturally occurring modification is present at a nucleotide position within in the [ASt Domain2] of SEQ ID NO: 4834.

211. The TREM of any one of the preceding embodiments, wherein the TREM comprises SEQ ID NO: 4834, the non-naturally occurring modification is selected from a 2′-O-methyl (2-modification, and the non-naturally occurring modification is present at a nucleotide position within in the [ASt Domain2].

212. The TREM of any one of the preceding embodiments, wherein the TREM comprises SEQ ID NO: 4834, the non-naturally occurring modification is a 2′-O-methyl (2-OMe) or 2′-halo (e.g., 2′F or 2′Cl) modification, and the non-naturally occurring modification is present at a nucleotide position within in the [ASt Domain2].

213. The TREM of any one of the preceding embodiments, wherein the TREM comprises SEQ ID NO: 4834 and does not contain a non-naturally occurring modification within in the [ASt Domain2].

214. The TREM of any one of the preceding embodiments, wherein the non-naturally occurring modification is present at a nucleotide position which corresponds to one or more of the nucleotides of SEQ ID NO: 6749.

215. The TREM of any one of the preceding embodiments, wherein:
(i) the non-naturally occurring modification is present at a nucleotide position which corresponds to one or more of the nucleotides of SEQ ID NO: 6749; and/or
(ii) the TREM differs from the nucleotide sequence of SEQ ID NO: 6749 by no more than 1, 2, 3, 4, 5, 6, 7, 8, 9, or 10 nucleotides.

216. The TREM of any one of the preceding embodiments, wherein:
(i) the non-naturally occurring modification is present at a nucleotide position which corresponds to one or more of the nucleotides of SEQ ID NO: 6749; and/or
(ii) the TREM comprises the nucleotide sequence of SEQ ID NO: 6749.

217. The TREM of any one of the preceding embodiments, wherein the TREM comprises SEQ ID NO: 6749 and the non-naturally occurring modification is present at a nucleotide position within in the [ASt Domain1] of SEQ ID NO: 6749.

218. The TREM of any one of the preceding embodiments, wherein the TREM comprises SEQ ID NO: 6749, the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification, and the non-naturally occurring modification is present at a nucleotide position within in the [ASt Domain1].

219. The TREM of any one of the preceding embodiments, wherein the TREM comprises SEQ ID NO: 6749, the non-naturally occurring modification is a 2′-O-methyl (2-OMe) modification, and the non-naturally occurring modification is present at a nucleotide position within in the [ASt Domain1].

220. The TREM of any one of the preceding embodiments, wherein the TREM comprises SEQ ID NO: 6749 and does not contain a non-naturally occurring modification within in the [ASt Domain1].

221. The TREM of any one of the preceding embodiments, wherein the TREM comprises SEQ ID NO: 6749 and the non-naturally occurring modification is present at a nucleotide position within in the [DH Domain] of SEQ ID NO: 6749.

222. The TREM of any one of the preceding embodiments, wherein the TREM comprises SEQ ID NO: 6749, the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification, and the non-naturally occurring modification is present at a nucleotide position within in the [DH Domain].

223. The TREM of any one of the preceding embodiments, wherein the TREM comprises SEQ ID NO: 6749, the non-naturally occurring modification is a 2′-O-methyl (2-OMe) or 2′-halo (e.g., 2′F or 2′Cl) modification, and the non-naturally occurring modification is present at a nucleotide position within in the [DH Domain].

224. The TREM of any one of the preceding embodiments, wherein the TREM comprises SEQ ID NO: 6749 and does not contain a non-naturally occurring modification within in the [DH Domain].

225. The TREM of any one of the preceding embodiments, wherein the TREM comprises SEQ ID NO: 6749 and the non-naturally occurring modification is present at a nucleotide position within in the [ACH Domain] of SEQ ID NO: 6749.

226. The TREM of any one of the preceding embodiments, wherein the TREM comprises SEQ ID NO: 6749, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [ACH Domain].

227. The TREM of any one of the preceding embodiments, wherein the TREM comprises SEQ ID NO: 6749, the non-naturally occurring modification is a 2′-O-methyl (2-OMe) or 2′-halo (e.g., 2′F or 2′Cl) modification, and the non-naturally occurring modification is present at a nucleotide position within in the [ACH Domain].

228. The TREM of any one of the preceding embodiments, wherein the TREM comprises SEQ ID NO: 6749 and does not contain a non-naturally occurring modification within in the [ACH Domain].

229. The TREM of any one of the preceding embodiments, wherein the TREM comprises SEQ ID NO: 6749 and the non-naturally occurring modification is present at a nucleotide position within in the [VL Domain] of SEQ ID NO: 6749.

230. The TREM of any one of the preceding embodiments, wherein the TREM comprises SEQ ID NO: 6749, the non-naturally occurring modification is selected from a 2′-O-methyl (2-modification, and the non-naturally occurring modification is present at a nucleotide position within in the [VL Domain].

231. The TREM of any one of the preceding embodiments, wherein the TREM comprises SEQ ID NO: 6749, the non-naturally occurring modification is a 2′-O-methyl (2-OMe) or 2′-halo (e.g., 2′F or 2′Cl) modification, and the non-naturally occurring modification is present at a nucleotide position within in the [VL Domain].

232. The TREM of any one of the preceding embodiments, wherein the TREM comprises SEQ ID NO: 6749 and does not contain a non-naturally occurring modification within in the [VL Domain].

233. The TREM of any one of the preceding embodiments, wherein the TREM comprises SEQ ID NO: 6749 and the non-naturally occurring modification is present at a nucleotide position within in the [TH Domain] of SEQ ID NO: 6749.

234. The TREM of any one of the preceding embodiments, wherein the TREM comprises SEQ ID NO: 6749, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [TH Domain].

235. The TREM of any one of the preceding embodiments, wherein the TREM comprises SEQ ID NO: 6749, the non-naturally occurring modification is a 2′-O-methyl (2-OMe) or 2′-halo (e.g., 2′F or 2′Cl) modification, and the non-naturally occurring modification is present at a nucleotide position within in the [TH Domain].

236. The TREM of any one of the preceding embodiments, wherein the TREM comprises SEQ ID NO: 6749 and does not contain a non-naturally occurring modification within in the [TH Domain].

237. The TREM of any one of the preceding embodiments, wherein the TREM comprises SEQ ID NO: 6749 and the non-naturally occurring modification is present at a nucleotide position within in the [ASt Domain2] of SEQ ID NO: 6749.

238. The TREM of any one of the preceding embodiments, wherein the TREM comprises SEQ ID NO: 6749, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [ASt Domain2].

239. The TREM of any one of the preceding embodiments, wherein the TREM comprises SEQ ID NO: 6749, the non-naturally occurring modification is a 2′-O-methyl (2-OMe) or 2′-halo (e.g., 2′F or 2′Cl) modification, and the non-naturally occurring modification is present at a nucleotide position within in the [ASt Domain2].

240. The TREM of any one of the preceding embodiments, wherein the TREM comprises SEQ ID NO: 6749 and does not contain a non-naturally occurring modification within in the [ASt Domain2].

241. The TREM of any one of the preceding embodiments, wherein the non-naturally occurring modification is present at a nucleotide position which corresponds to one or more of the nucleotides of SEQ ID NO: 8051.

242. The TREM of any one of the preceding embodiments, wherein:
(i) the non-naturally occurring modification is present at a nucleotide position which corresponds to one or more of the nucleotides of SEQ ID NO: 8051; and/or
(ii) the TREM differs from the nucleotide sequence of SEQ ID NO: 8051 by no more than 1, 2, 3, 4, 5, 6, 7, 8, 9, or 10 nucleotides.

243. The TREM of any one of the preceding embodiments, wherein:
(i) the non-naturally occurring modification is present at a nucleotide position which corresponds to one or more of the nucleotides of SEQ ID NO: 8051; and/or
(ii) the TREM comprises the nucleotide sequence of SEQ ID NO: 8051.

244. The TREM of any one of the preceding embodiments, wherein the TREM comprises SEQ ID NO: 8051 and the non-naturally occurring modification is present at a nucleotide position within in the [ASt Domain1] of SEQ ID NO: 8051.

245. The TREM of any one of the preceding embodiments, wherein the TREM comprises SEQ ID NO: 8051, the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification, and the non-naturally occurring modification is present at a nucleotide position within in the [ASt Domain1].

246. The TREM of any one of the preceding embodiments, wherein the TREM comprises SEQ ID NO: 8051, the non-naturally occurring modification is a 2′-O-methyl (2-OMe) modification, and the non-naturally occurring modification is present at a nucleotide position within in the [ASt Domain1].

247. The TREM of any one of the preceding embodiments, wherein the TREM comprises SEQ ID NO: 8051 and does not contain a non-naturally occurring modification within in the [ASt Domain1].

248. The TREM of any one of the preceding embodiments, wherein the TREM comprises SEQ ID NO: 6749 and the non-naturally occurring modification is present at a nucleotide position within in the [DH Domain] of SEQ ID NO: 8051.

249. The TREM of any one of the preceding embodiments, wherein the TREM comprises SEQ ID NO: 8051, the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification, and the non-naturally occurring modification is present at a nucleotide position within in the [DH Domain].

250. The TREM of any one of the preceding embodiments, wherein the TREM comprises SEQ ID NO: 8051, the non-naturally occurring modification is a 2′-O-methyl (2-OMe) or 2′-halo (e.g., 2′F or 2′Cl) modification, and the non-naturally occurring modification is present at a nucleotide position within in the [DH Domain].

251. The TREM of any one of the preceding embodiments, wherein the TREM comprises SEQ ID NO: 8051 and does not contain a non-naturally occurring modification within in the [DH Domain].

252. The TREM of any one of the preceding embodiments, wherein the TREM comprises SEQ ID NO: 8051 and the non-naturally occurring modification is present at a nucleotide position within in the [ACH Domain] of SEQ ID NO: 8051.

253. The TREM of any one of the preceding embodiments, wherein the TREM comprises SEQ ID NO: 8051, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [ACH Domain].

254. The TREM of any one of the preceding embodiments, wherein the TREM comprises SEQ ID NO: 8051, the non-naturally occurring modification is a 2′-O-methyl (2-OMe) or 2′-halo (e.g., 2′F or 2′Cl) modification, and the non-naturally occurring modification is present at a nucleotide position within in the [ACH Domain].

255. The TREM of any one of the preceding embodiments, wherein the TREM comprises SEQ ID NO: 8051 and does not contain a non-naturally occurring modification within in the [ACH Domain].

256. The TREM of any one of the preceding embodiments, wherein the TREM comprises SEQ ID NO: 8051 and the non-naturally occurring modification is present at a nucleotide position within in the [VL Domain] of SEQ ID NO: 8051.

257. The TREM of any one of the preceding embodiments, wherein the TREM comprises SEQ ID NO: 8051, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [VL Domain].

258. The TREM of any one of the preceding embodiments, wherein the TREM comprises SEQ ID NO: 8051, the non-naturally occurring modification is a 2′-O-methyl (2-OMe) or 2′-halo (e.g., 2′F or 2′Cl) modification, and the non-naturally occurring modification is present at a nucleotide position within in the [VL Domain].

259. The TREM of any one of the preceding embodiments, wherein the TREM comprises SEQ ID NO: 8051 and does not contain a non-naturally occurring modification within in the [VL Domain].

260. The TREM of any one of the preceding embodiments, wherein the TREM comprises SEQ ID NO: 8051 and the non-naturally occurring modification is present at a nucleotide position within in the [TH Domain] of SEQ ID NO: 8051.

261. The TREM of any one of the preceding embodiments, wherein the TREM comprises SEQ ID NO: 8051, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [TH Domain].

262. The TREM of any one of the preceding embodiments, wherein the TREM comprises SEQ ID NO: 8051, the non-naturally occurring modification is a 2′-O-methyl (2-OMe) or 2′-halo (e.g., 2′F or 2′Cl) modification, and the non-naturally occurring modification is present at a nucleotide position within in the [TH Domain].

263. The TREM of any one of the preceding embodiments, wherein the TREM comprises SEQ ID NO: 8051 and does not contain a non-naturally occurring modification within in the [TH Domain].

264. The TREM of any one of the preceding embodiments, wherein the TREM comprises SEQ ID NO: 8051 and the non-naturally occurring modification is present at a nucleotide position within in the [ASt Domain2] of SEQ ID NO: 8051.

265. The TREM of any one of the preceding embodiments, wherein the TREM comprises SEQ ID NO: 8051, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [ASt Domain2].

266. The TREM of any one of the preceding embodiments, wherein the TREM comprises SEQ ID NO: 8051, the non-naturally occurring modification is a 2′-O-methyl (2-OMe) or 2′-halo (e.g., 2′F or 2′Cl) modification, and the non-naturally occurring modification is present at a nucleotide position within in the [ASt Domain2].

267. The TREM of any one of the preceding embodiments, wherein the TREM comprises SEQ ID NO: 8051 and does not contain a non-naturally occurring modification within in the [ASt Domain2].

268. The TREM of any one of the preceding embodiments, wherein the non-naturally occurring modification is present at a nucleotide position which corresponds to one or more of the nucleotides of SEQ ID NO: 6707.

269. The TREM of any one of the preceding embodiments, wherein:
(i) the non-naturally occurring modification is present at a nucleotide position which corresponds to one or more of the nucleotides of SEQ ID NO: 6707; and/or
(ii) the TREM differs from the nucleotide sequence of SEQ ID NO: 6707 by no more than 1, 2, 3, 4, 5, 6, 7, 8, 9, or 10 nucleotides.

270. The TREM of any one of the preceding embodiments, wherein:
(i) the non-naturally occurring modification is present at a nucleotide position which corresponds to one or more of the nucleotides of SEQ ID NO: 6707; and/or
(ii) the TREM comprises the nucleotide sequence of SEQ ID NO: 6707.

271. The TREM of any one of the preceding embodiments, wherein the TREM comprises SEQ ID NO: 6707 and the non-naturally occurring modification is present at a nucleotide position within in the [ASt Domain1] of SEQ ID NO: 6707.

272. The TREM of any one of the preceding embodiments, wherein the TREM comprises SEQ ID NO: 6707, the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification, and the non-naturally occurring modification is present at a nucleotide position within in the [ASt Domain1].

273. The TREM of any one of the preceding embodiments, wherein the TREM comprises SEQ ID NO: 6707, the non-naturally occurring modification is a 2′-O-methyl (2-OMe) modification, and the non-naturally occurring modification is present at a nucleotide position within in the [ASt Domain1].

274. The TREM of any one of the preceding embodiments, wherein the TREM comprises SEQ ID NO: 6707 and does not contain a non-naturally occurring modification within in the [ASt Domain1].

275. The TREM of any one of the preceding embodiments, wherein the TREM comprises SEQ ID NO: 6707 and the non-naturally occurring modification is present at a nucleotide position within in the [DH Domain] of SEQ ID NO: 6707.

276. The TREM of any one of the preceding embodiments, wherein the TREM comprises SEQ ID NO: 6707, the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification, and the non-naturally occurring modification is present at a nucleotide position within in the [DH Domain].

277. The TREM of any one of the preceding embodiments, wherein the TREM comprises SEQ ID NO: 6707, the non-naturally occurring modification is a 2′-O-methyl (2-OMe) or 2′-halo (e.g., 2′F or 2′Cl) modification, and the non-naturally occurring modification is present at a nucleotide position within in the [DH Domain].

278. The TREM of any one of the preceding embodiments, wherein the TREM comprises SEQ ID NO: 6707 and does not contain a non-naturally occurring modification within in the [DH Domain].

279. The TREM of any one of the preceding embodiments, wherein the TREM comprises SEQ ID NO: 6707 and the non-naturally occurring modification is present at a nucleotide position within in the [ACH Domain] of SEQ ID NO: 6707.

280. The TREM of any one of the preceding embodiments, wherein the TREM comprises SEQ ID NO: 6707, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [ACH Domain].

281. The TREM of any one of the preceding embodiments, wherein the TREM comprises SEQ ID NO: 6707, the non-naturally occurring modification is a 2′-O-methyl (2-OMe) or 2′-halo (e.g., 2′F or 2′Cl) modification, and the non-naturally occurring modification is present at a nucleotide position within in the [ACH Domain].

282. The TREM of any one of the preceding embodiments, wherein the TREM comprises SEQ ID NO: 6707 and does not contain a non-naturally occurring modification within in the [ACH Domain].

283. The TREM of any one of the preceding embodiments, wherein the TREM comprises SEQ ID NO: 6707 and the non-naturally occurring modification is present at a nucleotide position within in the [VL Domain] of SEQ ID NO: 6707.

284. The TREM of any one of the preceding embodiments, wherein the TREM comprises SEQ ID NO: 6707, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [VL Domain].

285. The TREM of any one of the preceding embodiments, wherein the TREM comprises SEQ ID NO: 6707, the non-naturally occurring modification is a 2′-O-methyl (2-OMe) or 2′-halo (e.g., 2′F or 2′Cl) modification, and the non-naturally occurring modification is present at a nucleotide position within in the [VL Domain].

286. The TREM of any one of the preceding embodiments, wherein the TREM comprises SEQ ID NO: 6707 and does not contain a non-naturally occurring modification within in the [VL Domain].

287. The TREM of any one of the preceding embodiments, wherein the TREM comprises SEQ ID NO: 6707 and the non-naturally occurring modification is present at a nucleotide position within in the [TH Domain] of SEQ ID NO: 6707.

288. The TREM of any one of the preceding embodiments, wherein the TREM comprises SEQ ID NO: 6707, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [TH Domain].

289. The TREM of any one of the preceding embodiments, wherein the TREM comprises SEQ ID NO: 6707, the non-naturally occurring modification is a 2′-O-methyl (2-OMe) or 2′-halo (e.g., 2′F or 2′Cl) modification, and the non-naturally occurring modification is present at a nucleotide position within in the [TH Domain].

290. The TREM of any one of the preceding embodiments, wherein the TREM comprises SEQ ID NO: 6707 and does not contain a non-naturally occurring modification within in the [TH Domain].

291. The TREM of any one of the preceding embodiments, wherein the TREM comprises SEQ ID NO: 6707 and the non-naturally occurring modification is present at a nucleotide position within in the [ASt Domain2] of SEQ ID NO: 6707.

292. The TREM of any one of the preceding embodiments, wherein the TREM comprises SEQ ID NO: 6707, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [ASt Domain2].

293. The TREM of any one of the preceding embodiments, wherein the TREM comprises SEQ ID NO: 6707, the non-naturally occurring modification is a 2′-O-methyl (2-OMe) or 2′-halo (e.g., 2′F or 2′Cl) modification, and the non-naturally occurring modification is present at a nucleotide position within in the [ASt Domain2].

294. The TREM of any one of the preceding embodiments, wherein the TREM comprises SEQ ID NO: 6707 and does not contain a non-naturally occurring modification within in the [ASt Domain2].

295. The TREM of any one of the preceding embodiments, wherein the non-naturally occurring modification is present at a nucleotide position which corresponds to one or more of the nucleotides of SEQ ID NO: 5630.

296. The TREM of any one of the preceding embodiments, wherein:
(i) the non-naturally occurring modification is present at a nucleotide position which corresponds to one or more of the nucleotides of SEQ ID NO: 5630; and/or
(ii) the TREM differs from the nucleotide sequence of SEQ ID NO: 5630 by no more than 1, 2, 3, 4, 5, 6, 7, 8, 9, or 10 nucleotides.

297. The TREM of any one of the preceding embodiments, wherein:
(i) the non-naturally occurring modification is present at a nucleotide position which corresponds to one or more of the nucleotides of SEQ ID NO: 5630; and/or
(ii) the TREM comprises the nucleotide sequence of SEQ ID NO: 5630.

298. The TREM of any one of the preceding embodiments, wherein the TREM comprises SEQ ID NO: 5630 and the non-naturally occurring modification is present at a nucleotide position within in the [ASt Domain1] of SEQ ID NO: 5630.

299. The TREM of any one of the preceding embodiments, wherein the TREM comprises SEQ ID NO: 5630, the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification, and the non-naturally occurring modification is present at a nucleotide position within in the [ASt Domain1].

300. The TREM of any one of the preceding embodiments, wherein the TREM comprises SEQ ID NO: 5630, the non-naturally occurring modification is a 2′-O-methyl (2-OMe) modification, and the non-naturally occurring modification is present at a nucleotide position within in the [ASt Domain1].

301. The TREM of any one of the preceding embodiments, wherein the TREM comprises SEQ ID NO: 5630 and does not contain a non-naturally occurring modification within in the [ASt Domain1].

302. The TREM of any one of the preceding embodiments, wherein the TREM comprises SEQ ID NO: 5630 and the non-naturally occurring modification is present at a nucleotide position within in the [DH Domain] of SEQ ID NO: 5630.

303. The TREM of any one of the preceding embodiments, wherein the TREM comprises SEQ ID NO: 5630, the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification, and the non-naturally occurring modification is present at a nucleotide position within in the [DH Domain].

304. The TREM of any one of the preceding embodiments, wherein the TREM comprises SEQ ID NO: 5630, the non-naturally occurring modification is a 2′-O-methyl (2-OMe) or 2′-halo (e.g., 2′F or 2′Cl) modification, and the non-naturally occurring modification is present at a nucleotide position within in the [DH Domain].

305. The TREM of any one of the preceding embodiments, wherein the TREM comprises SEQ ID NO: 5630 and does not contain a non-naturally occurring modification within in the [DH Domain].

306. The TREM of any one of the preceding embodiments, wherein the TREM comprises SEQ ID NO: 5630 and the non-naturally occurring modification is present at a nucleotide position within in the [ACH Domain] of SEQ ID NO: 5630.

307. The TREM of any one of the preceding embodiments, wherein the TREM comprises SEQ ID NO: 5630, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [ACH Domain].

308. The TREM of any one of the preceding embodiments, wherein the TREM comprises SEQ ID NO: 5630, the non-naturally occurring modification is a 2′-O-methyl (2-OMe) or 2′-halo (e.g., 2′F or 2′Cl) modification, and the non-naturally occurring modification is present at a nucleotide position within in the [ACH Domain].

309. The TREM of any one of the preceding embodiments, wherein the TREM comprises SEQ ID NO: 5630 and does not contain a non-naturally occurring modification within in the [ACH Domain].

310. The TREM of any one of the preceding embodiments, wherein the TREM comprises SEQ ID NO: 5630 and the non-naturally occurring modification is present at a nucleotide position within in the [VL Domain] of SEQ ID NO: 5630.

311. The TREM of any one of the preceding embodiments, wherein the TREM comprises SEQ ID NO: 5630, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [VL Domain].

312. The TREM of any one of the preceding embodiments, wherein the TREM comprises SEQ ID NO: 5630, the non-naturally occurring modification is a 2′-O-methyl (2-OMe) or 2′-halo (e.g., 2′F or 2′Cl) modification, and the non-naturally occurring modification is present at a nucleotide position within in the [VL Domain].

313. The TREM of any one of the preceding embodiments, wherein the TREM comprises SEQ ID NO: 5630 and does not contain a non-naturally occurring modification within in the [VL Domain].

314. The TREM of any one of the preceding embodiments, wherein the TREM comprises SEQ ID NO: 5630 and the non-naturally occurring modification is present at a nucleotide position within in the [TH Domain] of SEQ ID NO: 5630.

315. The TREM of any one of the preceding embodiments, wherein the TREM comprises SEQ ID NO: 5630, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [TH Domain].

316. The TREM of any one of the preceding embodiments, wherein the TREM comprises SEQ ID NO: 5630, the non-naturally occurring modification is a 2′-O-methyl (2-OMe) or 2′-halo (e.g., 2′F or 2′Cl) modification, and the non-naturally occurring modification is present at a nucleotide position within in the [TH Domain].

317. The TREM of any one of the preceding embodiments, wherein the TREM comprises SEQ ID NO: 5630 and does not contain a non-naturally occurring modification within in the [TH Domain].

318. The TREM of any one of the preceding embodiments, wherein the TREM comprises SEQ ID NO: 5630 and the non-naturally occurring modification is present at a nucleotide position within in the [ASt Domain2] of SEQ ID NO: 5630.

319. The TREM of any one of the preceding embodiments, wherein the TREM comprises SEQ ID NO: 5630, the non-naturally occurring modification is selected from a 2′-O-methyl (2-modification, and the non-naturally occurring modification is present at a nucleotide position within in the [ASt Domain2].

320. The TREM of any one of the preceding embodiments, wherein the TREM comprises SEQ ID NO: 5630, the non-naturally occurring modification is a 2′-O-methyl (2-OMe) or 2′-halo (e.g., 2′F or 2′Cl) modification, and the non-naturally occurring modification is present at a nucleotide position within in the [ASt Domain2].

321. The TREM of any one of the preceding embodiments, wherein the TREM comprises SEQ ID NO: 5630 and does not contain a non-naturally occurring modification within in the [ASt Domain2].

322. The TREM of any one of the preceding embodiments, wherein the non-naturally occurring modification is present at a nucleotide position which corresponds to one or more of the nucleotides of SEQ ID NO: 4249.

323. The TREM of any one of the preceding embodiments, wherein:
(i) the non-naturally occurring modification is present at a nucleotide position which corresponds to one or more of the nucleotides of SEQ ID NO: 4249; and/or
(ii) the TREM differs from the nucleotide sequence of SEQ ID NO: 4249 by no more than 1, 2, 3, 4, 5, 6, 7, 8, 9, or 10 nucleotides.

324. The TREM of any one of the preceding embodiments, wherein:
(i) the non-naturally occurring modification is present at a nucleotide position which corresponds to one or more of the nucleotides of SEQ ID NO: 4249; and/or
(ii) the TREM comprises the nucleotide sequence of SEQ ID NO: 4249.

325. The TREM of any one of the preceding embodiments, wherein the TREM comprises SEQ ID NO: 4249 and the non-naturally occurring modification is present at a nucleotide position within in the [ASt Domain1] of SEQ ID NO: 4249.

326. The TREM of any one of the preceding embodiments, wherein the TREM comprises SEQ ID NO: 4249, the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification, and the non-naturally occurring modification is present at a nucleotide position within in the [ASt Domain1].

327. The TREM of any one of the preceding embodiments, wherein the TREM comprises SEQ ID NO: 4249, the non-naturally occurring modification is a 2′-O-methyl (2-OMe) modification, and the non-naturally occurring modification is present at a nucleotide position within in the [ASt Domain1].

328. The TREM of any one of the preceding embodiments, wherein the TREM comprises SEQ ID NO: 4249 and does not contain a non-naturally occurring modification within in the [ASt Domain1].

329. The TREM of any one of the preceding embodiments, wherein the TREM comprises SEQ ID NO: 4249 and the non-naturally occurring modification is present at a nucleotide position within in the [DH Domain] of SEQ ID NO: 4249.

330. The TREM of any one of the preceding embodiments, wherein the TREM comprises SEQ ID NO: 4249, the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification, and the non-naturally occurring modification is present at a nucleotide position within in the [DH Domain].

331. The TREM of any one of the preceding embodiments, wherein the TREM comprises SEQ ID NO: 4249, the non-naturally occurring modification is a 2′-O-methyl (2-OMe) or 2′-halo (e.g., 2′F or 2′Cl) modification, and the non-naturally occurring modification is present at a nucleotide position within in the [DH Domain].

332. The TREM of any one of the preceding embodiments, wherein the TREM comprises SEQ ID NO: 4249 and does not contain a non-naturally occurring modification within in the [DH Domain].

333. The TREM of any one of the preceding embodiments, wherein the TREM comprises SEQ ID NO: 4249 and the non-naturally occurring modification is present at a nucleotide position within in the [ACH Domain] of SEQ ID NO: 4249.

334. The TREM of any one of the preceding embodiments, wherein the TREM comprises SEQ ID NO: 4249, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [ACH Domain].

335. The TREM of any one of the preceding embodiments, wherein the TREM comprises SEQ ID NO: 4249, the non-naturally occurring modification is a 2′-O-methyl (2-OMe) or 2′-halo (e.g., 2′F or 2′Cl) modification, and the non-naturally occurring modification is present at a nucleotide position within in the [ACH Domain].

336. The TREM of any one of the preceding embodiments, wherein the TREM comprises SEQ ID NO: 4249 and does not contain a non-naturally occurring modification within in the [ACH Domain].

337. The TREM of any one of the preceding embodiments, wherein the TREM comprises SEQ ID NO: 4249 and the non-naturally occurring modification is present at a nucleotide position within in the [VL Domain] of SEQ ID NO: 4249.

338. The TREM of any one of the preceding embodiments, wherein the TREM comprises SEQ ID NO: 4249, the non-naturally occurring modification is selected from a 2′-O-methyl (2-modification, and the non-naturally occurring modification is present at a nucleotide position within in the [VL Domain].

339. The TREM of any one of the preceding embodiments, wherein the TREM comprises SEQ ID NO: 4249, the non-naturally occurring modification is a 2′-O-methyl (2-OMe) or 2′-halo (e.g., 2′F or 2′Cl) modification, and the non-naturally occurring modification is present at a nucleotide position within in the [VL Domain].

340. The TREM of any one of the preceding embodiments, wherein the TREM comprises SEQ ID NO: 4249 and does not contain a non-naturally occurring modification within in the [VL Domain].

341. The TREM of any one of the preceding embodiments, wherein the TREM comprises SEQ ID NO: 4249 and the non-naturally occurring modification is present at a nucleotide position within in the [TH Domain] of SEQ ID NO: 4249.

342. The TREM of any one of the preceding embodiments, wherein the TREM comprises SEQ ID NO: 4249, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [TH Domain].

343. The TREM of any one of the preceding embodiments, wherein the TREM comprises SEQ ID NO: 4249, the non-naturally occurring modification is a 2′-O-methyl (2-OMe) or 2′-halo (e.g., 2′F or 2′Cl) modification, and the non-naturally occurring modification is present at a nucleotide position within in the [TH Domain].

344. The TREM of any one of the preceding embodiments, wherein the TREM comprises SEQ ID NO: 4249 and does not contain a non-naturally occurring modification within in the [TH Domain].

345. The TREM of any one of the preceding embodiments, wherein the TREM comprises SEQ ID NO: 4249 and the non-naturally occurring modification is present at a nucleotide position within in the [ASt Domain2] of SEQ ID NO: 4249.

346. The TREM of any one of the preceding embodiments, wherein the TREM comprises SEQ ID NO: 4249, the non-naturally occurring modification is selected from a 2′-O-methyl (2-OMe), 2′-halo (e.g., 2′F or 2′Cl), 2′-O-methoxyethyl (2′MOE2′deoxy, or phosphorothioate modification, and the non-naturally occurring modification is present at a nucleotide position within in the [ASt Domain2].

347. The TREM of any one of the preceding embodiments, wherein the TREM comprises SEQ ID NO: 4249, the non-naturally occurring modification is a 2′-O-methyl (2-OMe) or 2′-halo (e.g., 2′F or 2′Cl) modification, and the non-naturally occurring modification is present at a nucleotide position within in the [ASt Domain2].

348. The TREM of any one of the preceding embodiments, wherein the TREM comprises SEQ ID NO: 4249 and does not contain a non-naturally occurring modification within in the [ASt Domain2].

349. The TREM of any one of the preceding embodiments, wherein the TREM comprises the sequence of Formula I_{ALA} (SEQ ID NO: 562):
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂, wherein R is a ribonucleotide residue and the consensus for Ala is: R₀=absent; R₁₄, R₅₇=are independently A or absent; R₂₆=A, C, G or absent; R₅, R₆, R₁₅, R₁₆, R₂₁, R₃₀, R₃₁, R₃₂, R₃₄, R₃₇, R₄₁, R₄₂, R₄₃, R₄₄, R₄₅, R₄₈, R₄₉, R₅₀, R₅₈, R₅₉, R₆₃, R₆₄, R₆₆, R₆₇=are independently N or absent; R₁, R₃₅, R₆₅=are independently A, C, U or absent; R₁, R₉, R₂₀, R₃₈, R₄₀, R₅₁, R₅₂, R₅₆=are independently A, G or absent; R₇, R₂₂, R₂₅, R₂₇, R₂₉, R₄₆, R₅₃, R₇₂=are independently A, G, U or absent; R₂₄, R₆₉=are independently A, U or absent; R₇₀, R₇₁=are independently C or absent; R₃, R₄=are independently C, G or absent; R₁₂, R₃₃, R₃₆, R₆₂, R₆₈=are independently C, G, U or absent; R₁₃, R₁₇, R₂₈, R₃₉, R₅₅, R₆₀, R₆₁=are independently C, U or absent; R₁₀, R₁₉, R₂₃=are independently G or absent; R₂=G, U or absent; R₈, R₁₈, R₅₄=are independently U or absent; [R₄₇]ₓ=N or absent; wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

350. The TREM of embodiment 349, wherein the TREM comprises a non-naturally occurring modification present at a nucleotide position within one of the [ASt Domain1], [DH Domain], [ACH Domain], [VL Domain], [TH Domain], or [ASt Domain2].

351. The TREM of embodiment 350, wherein the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification.

352. The TREM of any one of the preceding embodiments, wherein the TREM comprises the sequence of Formula II_{ALA} (SEQ ID NO: 563),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂ wherein R is a ribonucleotide residue and the consensus for Ala is:
R₀, R₁₈=are absent;
R₁₄, R₂₄, R₅₇=are independently A or absent;
R₁₅, R₂₆, R₆₄=are independently A, C, G or absent;
R₁₆, R₃₁, R₅₀, R₅₉=are independently N or absent;
R₁₁, R₃₂, R₃₇, R₄₁, R₄₃, R₄₅, R₄₉, R₆₅, R₆₆=are independently A, C, U or absent;
R₁, R₅, R₉, R₂₅, R₂₇, R₃₈, R₄₀, R₄₆, R₅₁, R₅₆=are independently A, G or absent;
R₇, R₂₂, R₂₉, R₄₂, R₄₄, R₅₃, R₆₃, R₇₂=are independently A, G, U or absent;
R₆, R₃₅, R₆₉=are independently A, U or absent;
R₅₅, R₆₀, R₇₀, R₇₁=are independently C or absent;
R₃=C, G or absent;
R₁₂, R₃₆, R₄₈=are independently C, G, U or absent;
R₁₃, R₁₇, R₂₈, R₃₀, R₃₄, R₃₉, R₅₅, R₆₁, R₆₂, R₆₇, R₆₈=are independently C, U or absent;
R₄, R₁₀, R₁₉, R₂₀, R₂₃, R₅₂=are independently G or absent;
R₂, R₈, R₃₃=are independently G, U or absent;
R₂₁, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

353. The TREM of embodiment 352, wherein the TREM comprises a non-naturally occurring modification present at a nucleotide position within one of the [ASt Domain1], [DH Domain], [ACH Domain], [VL Domain], [TH Domain], or [ASt Domain2].

354. The TREM of embodiment 353, wherein the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification.

355. The TREM of any one of the preceding embodiments, wherein the TREM comprises the sequence of Formula III_{ALA} (SEQ ID NO: 564),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ala is:
R₀, R₁₈=are absent;
R₁₄, R₂₄, R₅₇, R₇₂=are independently A or absent;
R₁₅, R₂₆, R₆₄=are independently A, C, G or absent;
R₁₆, R₃₁, R₅₀=are independently N or absent;
R₁₁, R₃₂, R₃₇, R₄₁, R₄₃, R₄₅, R₄₉, R₆₅, R₆₆=are independently A, C, U or absent;
R₅, R₉, R₂₅, R₂₇, R₃₈, R₄₀, R₄₆, R₅₁, R₅₆=are independently A, G or absent;
R₇, R₂₂, R₂₉, R₄₂, R₄₄, R₅₃, R₆₃=are independently A, G, U or absent;
R₆, R₃₅=are independently A, U or absent;
R₅₅, R₆₀, R₆₁, R₇₀, R₇₁=are independently C or absent;
R₁₂, R₄₈, R₅₉=are independently C, G, U or absent;
R₁₃, R₁₇, R₂₈, R₃₀, R₃₄, R₃₉, R₅₈, R₆₂, R₆₇, R₆₈=are independently C, U or absent;
R₁, R₂, R₃, R₄, R₁₀, R₁₉, R₂₀, R₂₃, R₅₂=are independently G or absent;
R₃₃, R₃₆=are independently G, U or absent;
R₈, R₂₁, R₅₄, R₆₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

356. The TREM of embodiment 355, wherein the TREM comprises a non-naturally occurring modification present at a nucleotide position within one of the [ASt Domain1], [DH Domain], [ACH Domain], [VL Domain], [TH Domain], or [ASt Domain2].

357. The TREM of embodiment 356, wherein the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification.

358. The TREM of any one of the preceding embodiments, wherein the TREM comprises the sequence of Formula I ARG (SEQ ID NO: 565),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Arg is:
R₅₇=A or absent;
R₀, R₂₇=are independently A, C, G or absent;
R₁, R₂, R₃, R₄, R₅, R₆, R₇, R₁₁, R₁₂, R₁₆, R₂₁, R₂₂, R₂₃, R₂₅, R₂₆, R₂₉, R₃₀, R₃₁, R₃₂, R₃₃, R₃₄, R₃₇, R₄₂, R₄₄, R₄₅,
R₄₆, R₄₈, R₄₉, R₅₀, R₅₁, R₅₈, R₆₂, R₆₃, R₆₄, R₆₅, R₆₆, R₆₇, R₆₈, R₆₉, R₇₀, R₇₁=are independently N or absent;
R₁₃, R₁₇, R₄₁=are independently A, C, U or absent;
R₁₉, R₂₀, R₂₄, R₄₀, R₅₆=are independently A, G or absent;
R₁₄, R₁₅, R₇₂=are independently A, G, U or absent;
R₁₈=A, U or absent;
R₃₈=C or absent;
R₃₅, R₄₃, R₆₁=are independently C, G, U or absent;
R₂₈, R₅₅, R₅₉, R₆₀=are independently C, U or absent;
R₀, R₁₀, R₅₂=are independently G or absent;
R₈, R₃₉=are independently G, U or absent;
R₃₆, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

359. The TREM of embodiment 358, wherein the TREM comprises a non-naturally occurring modification present at a nucleotide position within one of the [ASt Domain1], [DH Domain], [ACH Domain], [VL Domain], [TH Domain], or [ASt Domain2].

360. The TREM of embodiment 359, wherein the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification.

361. The TREM of any one of the preceding embodiments, wherein the TREM comprises the sequence of Formula II ARG (SEQ ID NO: 566),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Arg is:
R₁₈=absent;
R₂₄, R₅₇=are independently A or absent;
R₄₁=A, C or absent;
R₃, R₇, R₃₄, R₅₀=are independently A, C, G or absent;
R₂, R₅, R₆, R₁₂, R₂₆, R₃₂, R₃₇, R₄₄, R₅₈, R₆₆, R₆₇, R₆₈, R₇₀=are independently N or absent;
R₄₉, R₇₁=are independently A, C, U or absent;
R₁, R₁₅, R₁₉, R₂₅, R₂₇, R₄₀, R₄₅, R₄₆, R₅₆, R₇₂=are independently A, G or absent;
R₁₄, R₂₉, R₆₃=are independently A, G, U or absent;
R₁₆, R₂₁=are independently A, U or absent;
R₃₈, R₆₁=are independently C or absent;
R₃₃, R₄₈=are independently C, G or absent;
R₄, R₉, R₁₁, R₄₃, R₆₂, R₆₄, R₆₉=are independently C, G, U or absent;
R₁₃, R₂₂, R₂₈, R₃₀, R₃₁, R₃₅, R₅₅, R₆₀, R₆₅=are independently C, U or absent;
R₀, R₁₀, R₂₀, R₂₃, R₅₁, R₅₂=are independently G or absent;
R₈, R₃₉, R₄₂=are independently G, U or absent;
R₁₇, R₃₆, R₅₃, R₅₄, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

362. The TREM of embodiment 361, wherein the TREM comprises a non-naturally occurring modification present at a nucleotide position within one of the [ASt Domain1], [DH Domain], [ACH Domain], [VL Domain], [TH Domain], or [ASt Domain2].

363. The TREM of embodiment 362, wherein the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification.

364. The TREM of any one of the preceding embodiments, wherein the TREM comprises the sequence of Formula III ARG (SEQ ID NO: 567),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Arg is:
R₁₈=is absent;
R₁₅, R₂₁, R₂₄, R₄₁, R₅₇=are independently A or absent;
R₃₄, R₄₄=are independently A, C or absent;
R₃, R₅, R₅₅=are independently A, C, G or absent;
R₂, R₆, R₆₆, R₇₀=are independently N or absent;
R₃₇, R₄₉=are independently A, C, U or absent;
R₁, R₂₅, R₂₉, R₄₀, R₄₅, R₄₆, R₅₀=are independently A, G or absent;
R₁₄, R₆₃, R₆₈=are independently A, G, U or absent;
R₁₆=A, U or absent;
R₃₈, R₆₁=are independently C or absent;
R₇, R₁₁, R₁₂, R₂₆, R₄₈=are independently C, G or absent;
R₆₄, R₆₇, R₆₉=are independently C, G, U or absent;
R₄, R₁₃, R₂₂, R₂₈, R₃₀, R₃₁, R₃₅, R₄₃, R₅₅, R₆₀, R₆₂, R₆₅, R₇₁=are independently C, U or absent;
R₀, R₁₀, R₁₉, R₂₀, R₂₃, R₂₇, R₃₃, R₅₁, R₅₂, R₅₆, R₇₂=are independently G or absent;
R₈, R₉, R₃₂, R₃₉, R₄₂=are independently G, U or absent;
R₁₇, R₃₆, R₅₃, R₅₄, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

365. The TREM of embodiment 364, wherein the TREM comprises a non-naturally occurring modification present at a nucleotide position within one of the [ASt Domain1], [DH Domain], [ACH Domain], [VL Domain], [TH Domain], or [ASt Domain2].

366. The TREM of embodiment 365, wherein the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification.

367. The TREM of any one of the preceding embodiments, wherein the TREM comprises the sequence of Formula I ASN (SEQ ID NO: 568),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Asn is:
R₀, R₁₈=are absent;
R₄₁=A or absent;
R₁₄, R₄₈, R₅₆=are independently A, C, G or absent;
R₂, R₄, R₅, R₆, R₁₂, R₁₇, R₂₆, R₂₉, R₃₀, R₃₁, R₄₄, R₄₅, R₄₆, R₄₉, R₅₀, R₅₈, R₆₂, R₆₃, R₆₅, R₆₆, R₆₇, R₆₈, R₇₀, R₇₁=are independently N or absent;
R₁₁, R₁₃, R₂₂, R₄₂, R₅₅, R₅₉=are independently A, C, U or absent;
R₉, R₁₅, R₂₄, R₂₇, R₃₄, R₃₇, R₅₁, R₇₂=are independently A, G or absent;
R₁, R₇, R₂₅, R₆₉=are independently A, G, U or absent;
R₄₀, R₅₇=are independently A, U or absent;
R₆₀=C or absent;
R₃₃=C, G or absent;
R₂₁, R₃₂, R₄₃, R₆₄=are independently C, G, U or absent;
R₃, R₁₆, R₂₈, R₃₅, R₃₆, R₆₁=are independently C, U or absent;
R₁₀, R₁₉, R₂₀, R₅₂=are independently G or absent;
R₅₄=G, U or absent;
R₈, R₂₃, R₃₈, R₃₉, R₅₃=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

368. The TREM of embodiment 367, wherein the TREM comprises a non-naturally occurring modification present at a nucleotide position within one of the [ASt Domain1], [DH Domain], [ACH Domain], [VL Domain], [TH Domain], or [ASt Domain2].

369. The TREM of embodiment 368, wherein the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification.

370. The TREM of any one of the preceding embodiments, wherein the TREM comprises the sequence of Formula II ASN (SEQ ID NO: 569),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Asn is:
R₀, R₁₈=are absent
R₂₄, R₄₁, R₄₆, R₆₂=are independently A or absent;
R₅₉=A, C or absent;
R₁₄, R₅₆, R₆₆=are independently A, C, G or absent;
R₁₇, R₂₉=are independently N or absent;
R₁₁, R₂₆, R₄₂, R₅₅=are independently A, C, U or absent;
R₁, R₉, R₁₂, R₁₅, R₂₅, R₃₄, R₃₇, R₄₈, R$1, R₆₇, R₆₈, R₆₉, R₇₀, R₇₂=are independently A, G or absent;
R₄₄, R₄₅, R₅₅=are independently A, G, U or absent;
R₄₀, R₅₇=are independently A, U or absent;
R₅, R₂₈, R₆₀=are independently C or absent;
R₃₃, R₆₅=are independently C, G or absent;
R₂₁, R₄₃, R₇₁=are independently C, G, U or absent;
R₃, R₆, R₁₃, R₂₂, R₃₂, R₃₅, R₃₆, R₆₁, R₆₃, R₆₄=are independently C, U or absent;
R₇, R₁₀, R₁₉, R₂₀, R₂₇, R₄₉, R₅₂=are independently G or absent;
R₅₄=G, U or absent;
R₂, R₄, R₈, R₁₆, R₂₃, R₃₀, R₃₁, R₃₈, R₃₉, R₅₀, R₅₃=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

371. The TREM of embodiment 370, wherein the TREM comprises a non-naturally occurring modification present at a nucleotide position within one of the [ASt Domain1], [DH Domain], [ACH Domain], [VL Domain], [TH Domain], or [ASt Domain2].

372. The TREM of embodiment 371, wherein the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification.

373. The TREM of any one of the preceding embodiments, wherein the TREM comprises the sequence of Formula III ASN (SEQ ID NO: 570),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein R is a ribonucleotide residue and the consensus for Asn is:
R₀, R₁₈=are absent
R₂₄, R₄₀, R₄₁, R₄₆, R₆₂=are independently A or absent;
R₅₉=A, C or absent;
R₁₄, R₅₆, R₆₆=are independently A, C, G or absent;
R₁₁, R₂₆, R₄₂, R₅₅=are independently A, C, U or absent;
R₁, R₉, R₁₂, R₁₅, R₃₄, R₃₇, R₄₈, R₅₁, R₆₇, R₆₈, R₆₉, R₇₀=are independently A, G or absent;
R₄₄, R₄₅, R₅₅=are independently A, G, U or absent;
R₅₇=A, U or absent;
R₅, R₂₈, R₆₀=are independently C or absent;
R₃₃, R₆₅=are independently C, G or absent;
R₁₇, R₂₁, R₂₉=are independently C, G, U or absent;
R₃, R₆, R₁₃, R₂₂, R₃₂, R₃₅, R₃₆, R₄₃, R₆₁, R₆₃, R₆₄, R₇₁=are independently C, U or absent;
R₇, R₁₀, R₁₉, R₂₀, R₂₅, R₂₇, R₄₉, R₅₂, R₇₂=are independently G or absent;
R₅₄=G, U or absent;
R₂, R₄, R₈, R₁₆, R₂₃, R₃₀, R₃₁, R₃₈, R₃₉, R so, R₅₃=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

374. The TREM of embodiment 373, wherein the TREM comprises a non-naturally occurring modification present at a nucleotide position within one of the [ASt Domain1], [DH Domain], [ACH Domain], [VL Domain], [TH Domain], or [ASt Domain2].

375. The TREM of embodiment 374, wherein the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification.

376. The TREM of any one of the preceding embodiments, wherein the TREM comprises the sequence of Formula I ASP (SEQ ID NO: 571),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Asp is:
R₀=absent
R₂₄, R₇₁=are independently A, C or absent;
R₃₃, R₄₆=are independently A, C, G or absent;
R₂, R₃, R₄, R₅, R₆, R₁₂, R₁₆, R₂₂, R₂₆, R₂₉, R₃₁, R₃₂, R₄₄, R₄₈, R₄₉, R₅₈, R₆₃, R₆₄, R₆₆, R₆₇, R₆₈, R₆₉=are
independently N or absent;
R₁₃, R₂₁, R₃₄, R₄₁, R₅₇, R₆₅=are independently A, C, U or absent;
R₉, R₁₀, R₁₄, R₁₅, R₂₀, R₂₇, R₃₇, R₄₀, R$1, R₅₆, R₇₂=are independently A, G or absent;
R₇, R₂₅, R₄₂=are independently A, G, U or absent;
R₃₉=C or absent;
R₅₀, R₆₂=are independently C, G or absent;
R₃₀, R₄₃, R₄₅, R₅₅, R₇₀=are independently C, G, U or absent;
R₈, R₁₁, R₁₇, R₁₈, R₂₈, R₃₅, R₅₃, R₅₉, R₆₀, R₆₁=are independently C, U or absent;
R₁₉, R₅₂=are independently G or absent;
R₁=G, U or absent;
R₂₃, R₃₆, R₃₈, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

377. The TREM of embodiment 376, wherein the TREM comprises a non-naturally occurring modification present at a nucleotide position within one of the [ASt Domain1], [DH Domain], [ACH Domain], [VL Domain], [TH Domain], or [ASt Domain2].

378. The TREM of embodiment 377, wherein the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification.

379. The TREM of any one of the preceding embodiments, wherein the TREM comprises the sequence of Formula II ASP (SEQ ID NO: 572),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Asp is:
R₀, R₁₇, R₁₈, R₂₃=are independently absent;
R₀, R₄₀=are independently A or absent;
R₂₄, R₇₁=are independently A, C or absent;
R₆₇, R₆₈=are independently A, C, G or absent;
R₂, R₆, R₆₆=are independently N or absent;
R₅₇, R₆₃=are independently A, C, U or absent;
R₁₀, R₁₄, R₂₇, R₃₃, R₃₇, R₄₄, R₄₆, R₅₁, R₅₆, R₆₄, R₇₂=are independently A, G or absent;
R₇, R₁₂, R₂₆, R₆₅=are independently A, U or absent;
R₃₉, R₆₁, R₆₂=are independently C or absent;
R₃, R₃₁, R₄₅, R₇₀=are independently C, G or absent;
R₄, R₅, R₂₉, R₄₃, R₅₅=are independently C, G, U or absent;
R₈, R₁₁, R₁₃, R₃₀, R₃₂, R₃₄, R₃₅, R₄₁, R₄₈, R₅₃, R₅₉, R₆₀=are independently C, U or absent;
R₁₅, R₁₉, R₂₀, R₂₅, R₄₂, R₅₀, R₅₂=are independently G or absent;
R₁, R₂₂, R₄₉, R₅₈, R₆₉=are independently G, U or absent;
R₁₆, R₂₁, R₂₈, R₃₆, R₃₈, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

380. The TREM of embodiment 379, wherein the TREM comprises a non-naturally occurring modification present at a nucleotide position within one of the [ASt Domain1], [DH Domain], [ACH Domain], [VL Domain], [TH Domain], or [ASt Domain2].

381. The TREM of embodiment 380, wherein the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification.

382. The TREM of any one of the preceding embodiments, wherein the TREM comprises the sequence of Formula III ASP (SEQ ID NO: 573),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Asp is:
R₀, R₁₇, R₁₈, R₂₃=are absent
R₉, R₁₂, R₄₀, R₆₅, R₇₁=are independently A or absent;
R₂, R₂₄, R₅₇=are independently A, C or absent;
R₆, R₁₄, R₂₇, R₄₆, R₅₁, R₅₆, R₆₄, R₆₇, R₆₈=are independently A, G or absent;
R₃, R₃₁, R₃₅, R₃₉, R₆₁, R₆₂=are independently C or absent;
R₆₆=C, G or absent;
R₅, R₈, R₂₉, R₃₀, R₃₂, R₃₄, R₄₁, R₄₃, R₄₈, R₅₅, R₅₉, R₆₀, R₆₃=are independently C, U or absent;
R₁₀, R₁₅, R₁₉, R₂₀, R₂₅, R₃₃, R₃₇, R₄₂, R₄₄, R₄₅, R₄₉, R so, R $2, R₆₉, R₇₀, R₇₂=are independently G or absent;
R₂₂, R₅₅=are independently G, U or absent;
R₁, R₄, R₇, R₁₁, R₁₃, R₁₆, R₂₁, R₂₆, R₂₈, R₃₆, R₃₈, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

383. The TREM of embodiment 382, wherein the TREM comprises a non-naturally occurring modification present at a nucleotide position within one of the [ASt Domain1], [DH Domain], [ACH Domain], [VL Domain], [TH Domain], or [ASt Domain2].

384. The TREM of embodiment 383, wherein the non-naturally occurring modification is selected from a 2′-O-methyl (2-OMe), 2′-halo (e.g., 2′F or 2′Cl), 2′-O-methoxyethyl (2′MOE), 2′deoxy, or phosphorothioate modification.

385. The TREM of any one of the preceding embodiments, wherein:
(i) the TREM comprises the nucleotide sequence of SEQ ID NO: 701; and/or
(ii) the TREM differs from the nucleotide sequence of SEQ ID NO: 701 by no more than 1, 2, 3, 4, 5, 6, 7, 8, 9, or 10 nucleotides; and/or
(iii) the TREM comprises the sequence of TREM NO.: 80; and/or
(iv) the TREM differs from the sequence of TREM NO: 80 by no more than 1, 2, 3, 4, 5, 6, 7, 8, 9, or 10 non-naturally occurring modifications, e.g., a non-naturally occurring modification selected from a 2′-O-methyl (2-OMe), 2′-halo (e.g., 2′F or 2′Cl), 2′-O-methoxyethyl (2′MOE2′deoxy, or phosphorothioate modification.

386. The TREM of any one of the preceding embodiments, wherein:
(i) the non-naturally occurring modification is present at a nucleotide position which corresponds to one or more of the nucleotides of SEQ ID NO: 701; and/or
(ii) the TREM comprises the nucleotide sequence of SEQ ID NO: 701.

387. The TREM of any one of the preceding embodiment, having the sequence of SEQ ID NO: 701.

388. The TREM of any one of the preceding embodiments, wherein:
(i) the TREM comprises the nucleotide sequence of SEQ ID NO: 2951; and/or
(ii) the TREM differs from the nucleotide sequence of SEQ ID NO: 2951 by no more than 1, 2, 3, 4, 5, 6, 7, 8, 9, or 10 nucleotides; and/or
(iii) the TREM comprises the sequence of TREM NO.: 2330; and/or
(iv) the TREM differs from the sequence of TREM NO: 2330 by no more than 1, 2, 3, 4, 5, 6, 7, 8, 9, or 10 non-naturally occurring modifications, e.g., a non-naturally occurring modification selected from a 2′-O-methyl (2-OMe), 2′-halo (e.g., 2′F or 2′Cl), 2′-O-methoxyethyl (2′MOE2′deoxy, or phosphorothioate modification.

388. The TREM of any one of the preceding embodiments, wherein:
(i) the non-naturally occurring modification is present at a nucleotide position which corresponds to one or more of the nucleotides of SEQ ID NO: 2951; and/or
(ii) the TREM comprises the nucleotide sequence of SEQ ID NO: 2951.

389. The TREM of any one of the preceding embodiment, having the sequence of SEQ ID NO: 2951.

390. The TREM of any one of the preceding embodiments, wherein:
(i) the TREM comprises the nucleotide sequence of SEQ ID NO: 6047; and/or
(ii) the TREM differs from the nucleotide sequence of SEQ ID NO: 6047 by no more than 1, 2, 3, 4, 5, 6, 7, 8, 9, or 10 nucleotides; and/or
(iii) the TREM comprises the sequence of TREM NO.: 5426; and/or
(iv) the TREM differs from the sequence of TREM NO: 5426 by no more than 1, 2, 3, 4, 5, 6, 7, 8, 9, or 10 non-naturally occurring modifications, e.g., a non-naturally occurring modification selected from a 2′-O-methyl (2-OMe), 2′-halo (e.g., 2′F or 2′Cl), 2′-O-methoxyethyl (2′MOE2′deoxy, or phosphorothioate modification.

391. The TREM of any one of the preceding embodiments, wherein:
(i) the non-naturally occurring modification is present at a nucleotide position which corresponds to one or more of the nucleotides of SEQ ID NO: 6047; and/or
(ii) the TREM comprises the nucleotide sequence of SEQ ID NO: 6047.

392. The TREM of any one of the preceding embodiment, having the sequence of SEQ ID NO: 6047.

393. The TREM of any one of the preceding embodiments, wherein:
(i) the TREM comprises the nucleotide sequence of SEQ ID NO: 9364; and/or
(ii) the TREM differs from the nucleotide sequence of SEQ ID NO: 9364 by no more than 1, 2, 3, 4, 5, 6, 7, 8, 9, or 10 nucleotides; and/or
(iii) the TREM comprises the sequence of TREM NO.: 8743; and/or
(iv) the TREM differs from the sequence of TREM NO: 8743 by no more than 1, 2, 3, 4, 5, 6, 7, 8, 9, or 10 non-naturally occurring modifications, e.g., a non-naturally occurring modification selected from a 2′-O-methyl (2-OMe), 2′-halo (e.g., 2′F or 2′Cl), 2′-O-methoxyethyl (2′MOE2′deoxy, or phosphorothioate modification.

394. The TREM of any one of the preceding embodiments, wherein:
(i) the non-naturally occurring modification is present at a nucleotide position which corresponds to one or more of the nucleotides of SEQ ID NO: 9364; and/or
(ii) the TREM comprises the nucleotide sequence of SEQ ID NO: 9364.

395. The TREM of any one of the preceding embodiment, having the sequence of SEQ ID NO: 9364.

396. The TREM of any one of the preceding embodiments, wherein:
(i) the TREM comprises the nucleotide sequence of SEQ ID NO: 3795; and/or
(ii) the TREM differs from the nucleotide sequence of SEQ ID NO: 3795 by no more than 1, 2, 3, 4, 5, 6, 7, 8, 9, or 10 nucleotides; and/or
(iii) the TREM comprises the sequence of TREM NO.: 3174; and/or
(iv) the TREM differs from the sequence of TREM NO: 3174 by no more than 1, 2, 3, 4, 5, 6, 7, 8, 9, or 10 non-naturally occurring modifications, e.g., a non-naturally occurring modification selected from a 2′-O-methyl (2-OMe), 2′-halo (e.g., 2′F or 2′Cl), 2′-O-methoxyethyl (2′MOE2′deoxy, or phosphorothioate modification.

397. The TREM of any one of the preceding embodiments, wherein:
(i) the non-naturally occurring modification is present at a nucleotide position which corresponds to one or more of the nucleotides of SEQ ID NO: 3795; and/or
(ii) the TREM comprises the nucleotide sequence of SEQ ID NO: 3795.

398. The TREM of any one of the preceding embodiment, having the sequence of SEQ ID NO: 3795.

399. The TREM of any one of the preceding embodiments, wherein:
(i) the TREM comprises the nucleotide sequence of SEQ ID NO: 8524; and/or
(ii) the TREM differs from the nucleotide sequence of SEQ ID NO: 8524 by no more than 1, 2, 3, 4, 5, 6, 7, 8, 9, or 10 nucleotides; and/or
(iii) the TREM comprises the sequence of TREM NO.: 7903; and/or
(iv) the TREM differs from the sequence of TREM NO: 7903 by no more than 1, 2, 3, 4, 5, 6, 7, 8, 9, or 10 non-naturally occurring modifications, e.g., a non-naturally occurring modification selected from a 2′-O-methyl (2-OMe), 2′-halo (e.g., 2′F or 2′Cl), 2′-O-methoxyethyl (2′MOE2′deoxy, or phosphorothioate modification.

400. The TREM of any one of the preceding embodiments, wherein:
(i) the non-naturally occurring modification is present at a nucleotide position which corresponds to one or more of the nucleotides of SEQ ID NO: 8524; and/or
(ii) the TREM comprises the nucleotide sequence of SEQ ID NO: 8524.

401. The TREM of any one of the preceding embodiment, having the sequence of SEQ ID NO: 8524.

402. The TREM of any one of the preceding embodiments, wherein:
(i) the TREM comprises the nucleotide sequence of SEQ ID NO: 6725; and/or
(ii) the TREM differs from the nucleotide sequence of SEQ ID NO: 6725 by no more than 1, 2, 3, 4, 5, 6, 7, 8, 9, or 10 nucleotides; and/or
(iii) the TREM comprises the sequence of TREM NO.: 6104; and/or
(iv) the TREM differs from the sequence of TREM NO: 6104 by no more than 1, 2, 3, 4, 5, 6, 7, 8, 9, or 10 non-naturally occurring modifications, e.g., a non-naturally occurring modification selected from a 2′-O-methyl (2-OMe), 2′-halo (e.g., 2′F or 2′Cl), 2′-O-methoxyethyl (2′MOE2′deoxy, or phosphorothioate modification.

403. The TREM of any one of the preceding embodiments, wherein:
(i) the non-naturally occurring modification is present at a nucleotide position which corresponds to one or more of the nucleotides of SEQ ID NO: 6725; and/or
(ii) the TREM comprises the nucleotide sequence of SEQ ID NO: 6725.

404. The TREM of any one of the preceding embodiment, having the sequence of SEQ ID NO: 6725.

405. The TREM of any one of the preceding embodiments, wherein:
(i) the TREM comprises the nucleotide sequence of SEQ ID NO: 8712; and/or
(ii) the TREM differs from the nucleotide sequence of SEQ ID NO: 8712 by no more than 1, 2, 3, 4, 5, 6, 7, 8, 9, or 10 nucleotides; and/or
(iii) the TREM comprises the sequence of TREM NO.: 8091; and/or
(iv) the TREM differs from the sequence of TREM NO: 8091 by no more than 1, 2, 3, 4, 5, 6, 7, 8, 9, or 10 non-naturally occurring modifications, e.g., a non-naturally occurring modification selected from a 2′-O-methyl (2-OMe), 2′-halo (e.g., 2′F or 2′Cl), 2′-O-methoxyethyl (2′MOE2′deoxy, or phosphorothioate modification.

406. The TREM of any one of the preceding embodiments, wherein:
(i) the non-naturally occurring modification is present at a nucleotide position which corresponds to one or more of the nucleotides of SEQ ID NO: 8712; and/or
(ii) the TREM comprises the nucleotide sequence of SEQ ID NO: 8712.

407. The TREM of any one of the preceding embodiment, having the sequence of SEQ ID NO: 8712.

408. The TREM of any one of the preceding embodiments, wherein:
(i) the TREM comprises the nucleotide sequence of SEQ ID NO: 9488; and/or
(ii) the TREM differs from the nucleotide sequence of SEQ ID NO: 9488 by no more than 1, 2, 3, 4, 5, 6, 7, 8, 9, or 10 nucleotides; and/or
(iii) the TREM comprises the sequence of TREM NO.: 8867; and/or
(iv) the TREM differs from the sequence of TREM NO: 8867 by no more than 1, 2, 3, 4, 5, 6, 7, 8, 9, or 10 non-naturally occurring modifications, e.g., a non-naturally occurring modification selected from a 2′-O-methyl (2-OMe), 2′-halo (e.g., 2′F or 2′Cl), 2′-O-methoxyethyl (2′MOE2′deoxy, or phosphorothioate modification.

409. The TREM of any one of the preceding embodiments, wherein:
(i) the non-naturally occurring modification is present at a nucleotide position which corresponds to one or more of the nucleotides of SEQ ID NO: 9488; and/or
(ii) the TREM comprises the nucleotide sequence of SEQ ID NO: 9488.

410. 9488 TREM of any one of the preceding embodiment, having the sequence of SEQ ID NO: 8712.

411. The TREM of any one of the preceding embodiments, wherein:
(i) the TREM comprises the nucleotide sequence of SEQ ID NO: 5397; and/or
(ii) the TREM differs from the nucleotide sequence of SEQ ID NO: 5397 by no more than 1, 2, 3, 4, 5, 6, 7, 8, 9, or 10 nucleotides; and/or
(iii) the TREM comprises the sequence of TREM NO.: 4776; and/or
(iv) the TREM differs from the sequence of TREM NO: 4776 by no more than 1, 2, 3, 4, 5, 6, 7, 8, 9, or 10 non-naturally occurring modifications, e.g., a non-naturally occurring modification selected from a 2′-O-methyl (2-OMe), 2′-halo (e.g., 2′F or 2′Cl), 2′-O-methoxyethyl (2′MOE2′deoxy, or phosphorothioate modification.

412. The TREM of any one of the preceding embodiments, wherein:
(i) the non-naturally occurring modification is present at a nucleotide position which corresponds to one or more of the nucleotides of SEQ ID NO: 5397; and/or
(ii) the TREM comprises the nucleotide sequence of SEQ ID NO: 5397.

413. 9488 TREM of any one of the preceding embodiment, having the sequence of SEQ ID NO: 8712.

414. The TREM of any one of the preceding embodiments, wherein the non-naturally occurring modification is present at a nucleotide position which corresponds to one or more of nucleotides according to the CtNS.

415. A pharmaceutical composition comprising a TREM of embodiments 1-414.

416. The pharmaceutical composition of embodiment 415, comprising a pharmaceutically acceptable component, e.g., an excipient.

417. A lipid nanoparticle formulation comprising a TREM of embodiments 1-414.

418. A method of making a TREM of embodiments 1-414, comprising linking a first nucleotide to a second nucleotide to form the TREM.

419. The method of embodiment 418, wherein the TREM, TREM core fragment or TREM fragment is synthetic (e.g., non-naturally occurring).

420. The method of embodiment 418-419, wherein the synthesis is performed in vitro.

421. The method of embodiment 419, wherein the TREM, TREM core fragment or TREM fragment is made by cell-free solid phase synthesis.

422. A cell comprising a TREM of embodiments 1-414.

423. A cell comprising a TREM, TREM core fragment or TREM fragment made according to the method of embodiment 418.

424 . . . . A method of modulating a tRNA pool in a cell comprising:
providing a TREM of embodiments 1-414, and
contacting the cell with the TREM,
thereby modulating the tRNA pool in the cell.

425. A method of contacting a cell, tissue, or subject with a TREM of embodiments 1-414, comprising contacting the cell, tissue or subject with the TREM, thereby contacting the cell, tissue, or subject with the TREM.

426. A method of presenting a TREM, comprising contacting the cell, tissue or subject with a TREM of embodiments 1-414, thereby presenting the TREM to a cell, tissue, or subject.

427. A method of forming a TREM-contacted cell, tissue, or subject, comprising contacting the cell, tissue or subject with a TREM of embodiments 1-414, thereby forming a TREM-contacted cell, tissue, or subject.

428. A method of using a TREM comprising, contacting the cell, tissue or subject with a TREM of embodiments 1-414, thereby using the TREM.

429. A method of applying a TREM to a cell, tissue, or subject, comprising contacting the cell, tissue or subject with a TREM of embodiments 1-414, thereby applying a TREM to a cell, tissue, or subject.

430. A method of exposing a cell, tissue, or subject to a TREM, comprising contacting the cell, tissue or subject with a TREM of embodiments 1-414, thereby exposing a cell, tissue, or subject to a TREM.

431. A method of forming an admixture of a TREM and a cell, tissue, or subject, comprising
contacting the cell, tissue or subject with a TREM of embodiments 1-414, thereby forming an admixture of a TREM and a cell, tissue, or subject.

432. A method of delivering a TREM to a cell, tissue, or subject, comprising:
providing a cell, tissue, or subject, and contacting the cell, tissue, or subject, a TREM of embodiments 1-414.

433. A method, e.g., an ex vivo method, of modulating the metabolism, e.g., the translational capacity of an organelle, comprising:
providing a preparation of an organelle, e.g., mitochondria or chloroplasts, and contacting the organelle with a TREM of embodiments 1-414.

434. A method of treating a subject, e.g., modulating the metabolism, e.g., the translational capacity of a cell, in a subject, comprising:
providing, e.g., administering to the subject a TREM of embodiments 1-414, thereby treating the subject.

435. A method of modulating a tRNA pool in a cell comprising an endogenous open reading frame (ORF), which ORF comprises a codon having a first sequence, comprising:
optionally, acquiring knowledge of the abundance of one or both of (i) and (ii), e.g., acquiring knowledge of the relative amounts of: (i) and (ii) in the cell, wherein (i) is a tRNA moiety having an anticodon that pairs with the codon of the ORF having a first sequence (the first tRNA moiety) and (ii) is an isoacceptor tRNA moiety having an anticodon that pairs with a codon other than the codon having the first sequence (the second tRNA moiety) in the cell;
contacting the cell with a TREM of embodiments 1-414, wherein the TREM has an anticodon that pairs with: the codon having the first sequence; or the codon other than the codon having the first sequence, in an amount and/or for a time sufficient to modulate the relative amounts of the first tRNA moiety and the second tRNA moiety in the cell,
thereby modulating the tRNA pool in the cell.

436. A method of modulating a tRNA pool in a subject having an endogenous open reading frame (ORF), which ORF comprises a codon having a first sequence, comprising:
optionally, acquiring knowledge of the abundance of one or both of (i) and (ii), e.g., acquiring knowledge of the relative amounts of: (i) and (ii) in the subject, wherein (i) is a tRNA moiety having an anticodon that pairs with the codon of the ORF having a first sequence (the first tRNA moiety) and (ii) is an isoacceptor tRNA moiety having an anticodon that pairs with a codon other than the codon having the first sequence (the second tRNA moiety) in the subject;
contacting the subject with a TREM of embodiments 1-414, wherein the TREM has an anticodon that pairs with: the codon having the first sequence; or the codon other than the codon having the first sequence, in an amount and/or for a time sufficient to modulate the relative amounts of the first tRNA moiety and the second tRNA moiety in the subject,
thereby modulating the tRNA pool in the subject.

437. A method of modulating a tRNA pool in a subject having an endogenous open reading frame (ORF) comprising a codon comprising a synonymous mutation (a synonymous mutation codon or SMC), comprising:
providing a composition comprising a TREM of embodiments 1-414, wherein the TREM comprises an isoacceptor tRNA moiety comprising an anticodon sequence that pairs with the SMC (the TREM);
contacting the subject with the composition in an amount and/or for a time sufficient to modulate the tRNA pool in the subject,
thereby modulating the tRNA pool in the subject.

438. A method of modulating a tRNA pool in a cell comprising an endogenous open reading frame (ORF) comprising a codon comprising a synonymous mutation (a synonymous mutation codon or SMC), comprising:
providing a composition comprising a TREM of embodiments 1-414, wherein the TREM comprises an isoacceptor tRNA moiety comprising an anticodon sequence that pairs with the SMC (the TREM);
contacting the cell with the composition comprising a TREM in an amount and/or for a time sufficient to modulate the tRNA pool in the cell,
thereby modulating the tRNA pool in the cell.

439. A method of modulating expression of a protein in a cell, wherein the protein is encoded by a nucleic acid comprising an endogenous open reading frame (ORF), which ORF comprises a codon having a mutation, comprising:
contacting the cell with a composition comprising a TREM of embodiments 1-414 in an amount and/or for a time sufficient to modulate expression of the encoded protein,
wherein the TREM has an anticodon that pairs with the codon having the mutation,
thereby modulating expression of the protein in the cell.

440. A method of modulating expression of a protein in a subject, wherein the protein is encoded by a nucleic acid comprising an endogenous open reading frame (ORF), which ORF comprises a codon having a mutation, comprising:
contacting the subject with a composition comprising a TREM of embodiments 1-414, in an amount and/or for a time sufficient to modulate expression of the encoded protein,
wherein the TREM has an anticodon that pairs with the codon having the mutation,
thereby modulating expression of the protein in the subject.

441. The method of embodiment 439-440, wherein the mutation in the ORF is a nonsense mutation, e.g., resulting in a premature stop codon chosen from UAA, UGA or UAG.

442. The method of embodiment 439-441, wherein the TREM comprises an anticodon that pairs with a stop codon.

443. The method of any one of the preceding embodiments, wherein the TREM comprises an anticodon that pairs with a stop codon.

Other features, objects, and advantages of the invention will be apparent from the description and from the claims.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In addition, the materials, methods, and examples are illustrative only and not intended to be limiting.

### DETAILED DESCRIPTION OF CERTAIN EMBODIMENTS

The present disclosure features tRNA-based effector molecules (TREMs) comprising a non-naturally occurring modification and methods relating thereto. As disclosed herein, TREMs are complex molecules which can mediate a variety of cellular processes. Pharmaceutical TREM compositions, e.g., TREMs comprising a non-naturally occurring modification, can be administered to a cell, a tissue, or to a subject to modulate these functions. Also disclosed herein are methods of modulating expression of a protein in a subject or cell, wherein the protein is encoded by a nucleic acid comprising an endogenous open reading frame (ORF) having a first sequence, e.g., a mutation, e.g., a premature termination codon (PTC), and methods of treating a subject having an endogenous open reading frame (ORF) which comprises a premature termination codon (PTC). Further disclosed herein are TREMs comprising a non-naturally occurring modification, methods of making the same and compositions thereof.

### Definitions

“Acquire” or “acquiring” as the terms are used herein, refer to obtaining possession of a value, e.g., a numerical value, by “directly acquiring” or “indirectly acquiring” the physical entity or value. “Directly acquiring” refers to performing a process (e.g., performing an analytical method) to obtain the value. “Indirectly acquiring” refers to receiving the value from another party or source (e.g., a third party laboratory that directly acquired the or value).

A “disease or disorder associated with a PTC” as that term is used herein includes, but is not limited to, a disease or disorder in which cells express, or at one time expressed, a polypeptide encoded by an ORF comprising a PTC. In some embodiments, a disease associated with a PTC is chosen from: a proliferative disorder (e.g., a cancer), a genetic disorder, a metabolic disorder, an immune disorder, an inflammatory disorder or a neurological disorder. Exemplary diseases or disorders associated with a PTC are provided in any one of Tables 15, 16 and 17. In an embodiment, the disease associated with a PTC is a cancer. In an embodiment, the disease associated with a PTC is a monogenic disease.

An “isoacceptor,” as that term is used herein, refers to a plurality of tRNA molecule or TREMs wherein each molecule of the plurality comprises a different naturally occurring anticodon sequence and each molecule of the plurality mediates the incorporation of the same amino acid and that amino acid is the amino acid that naturally corresponds to the anticodons of the plurality.

A “modification,” as that term is used herein with reference to a nucleotide, refers to a modification of the chemical structure, e.g., a covalent modification, of the subject nucleotide. The modification can be naturally occurring or non-naturally occurring. In an embodiment, the modification is non-naturally occurring. In an embodiment, the modification is naturally occurring. In an embodiment, the modification is a synthetic modification. In an embodiment, the modification is a modification provided in Tables 5, 6, 7, 8 or 9.

A “naturally occurring nucleotide.” as that term is used herein, refers to a nucleotide that does not comprise a non-naturally occurring modification. In an embodiment, it includes a naturally occurring modification.

A “non-naturally occurring modification,” as that term is used herein with reference to a nucleotide, refers to a modification that: (a) a cell, e.g., a human cell, does not make on an endogenous tRNA; or (b) a cell, e.g., a human cell, can make on an endogenous tRNA but wherein such modification is in a location in which it does not occur on a native tRNA, e.g., the modification is in a domain, linker or arm, or on a nucleotide and/or at a position within a domain, linker or arm, which does not have such modification in nature. In either case, the modification is added synthetically, e.g., in a cell free reaction, e.g., in a solid state or liquid phase synthetic reaction. In an embodiment, the non-naturally occurring modification is a modification that is not present (in identity, location or position) if a sequence of the TREM is expressed in a mammalian cell, e.g., a HEK293 cell line. Exemplary non-naturally occurring modifications are found in Tables 5, 6, 7, 8 or 9.

A “non-naturally modified nucleotide,” as that term is used herein, refers a nucleotide comprising a non-naturally occurring modification on or of a sugar, nucleobase, or phosphate moiety.

A “nucleotide,” as that term is used herein, refers to an entity comprising a sugar, typically a pentameric sugar; a nucleobase; and a phosphate linking group. In an embodiment, a nucleotide comprises a naturally occurring, e.g., naturally occurring in a human cell, nucleotide, e.g., an adenine, thymine, guanine, cytosine, or uracil nucleotide.

A “premature termination codon” or “PTC” as those terms are used herein, refer to a stop codon that occurs in an open reading frame (ORF) of a DNA or mRNA. In an embodiment, a PTC occurs at a position upstream of a naturally occurring stop codon in an ORF. In an embodiment, a PTC that occurs upstream of a naturally occurring stop codon, e.g., in an ORF, results in modulation of a production parameter of the corresponding mRNA or polypeptide encoded by the ORF. In an embodiment, a PTC can differ (or arise) from a pre-mutation sequence by a point mutation, e.g., a nonsense mutation. In an embodiment, a PTC can differ (or arise) from a pre-mutation sequence by a genetic change, e.g., abnormality, other than a point mutation, e.g., a frameshift, a deletion, an insertion, a rearrangement, an inversion, a translocation, a duplication, or a transversion. In an embodiment, a PTC results in the production of a truncated protein which lacks a native activity or which is associated with a mutant, disease, or other unwanted phenotype. In an embodiment, the ORF comprising the PTC is an ORF from a tumor suppressor gene. In an embodiment, the mutation giving rise to the PTC is a driver mutation, e.g., a mutation that provides a growth advantage to a tumor cell.

A “production parameter,” refers to an expression parameter and/or a signaling parameter. In an embodiment a production parameter is an expression parameter. An expression parameter includes an expression parameter of a polypeptide or protein encoded by the endogenous ORF having a first sequence or PTC; or an expression parameter of an RNA, e.g., messenger RNA, encoded by the endogenous ORF having a first sequence or PTC. In an embodiment, an expression parameter can include:
(a) protein translation;
(b) expression level (e.g., of polypeptide or protein, or mRNA);
(c) post-translational modification of polypeptide or protein;
(d) folding (e.g., of polypeptide or protein, or mRNA),
(e) structure (e.g., of polypeptide or protein, or mRNA),
(f) transduction (e.g., of polypeptide or protein),
(g) compartmentalization (e.g., of polypeptide or protein, or mRNA),
(h) incorporation (e.g., of polypeptide or protein, or mRNA) into a supermolecular structure, e.g., incorporation into a membrane, proteasome, or ribosome,
(i) incorporation into a multimeric polypeptide, e.g., a homo or heterodimer, and/or
(j) stability.

In an embodiment, a production parameter is a signaling parameter. A signaling parameter can include:
(1) modulation of a signaling pathway, e.g., a cellular signaling pathway which is downstream or upstream of the protein encoded by the endogenous ORF having a first sequence or PTC;
(2) cell fate modulation;
(3) ribosome occupancy modulation;
(4) protein translation modulation;
(5) mRNA stability modulation;
(6) protein folding and structure modulation;
(7) protein transduction or compartmentalization modulation; and/or
(8) protein stability modulation.

An “ORF having a PTC” as that phrase is used herein, refers to an open reading frame (ORF) which comprises a premature termination codon (PTC). In an embodiment, the ORF having the PTC is associated with a disease or disorder associated with a PTC, e.g., as described herein, e.g., a disease or disorder listed in any one of Tables 15, 16 and 17. In an embodiment, the ORF having the PTC is not associated with a disease or disorder associated with a PTC.

A “stop codon” as that term is used herein, refers to a three nucleotide contiguous sequence within messenger RNA that specifies a termination of translation. For example, UAG, UAA, UGA (in RNA) and TAG, TAA or TGA (in DNA) are stop codons. The stop codons are also known as amber (UAG), ochre (UAA), and opal (UGA).

A “tRNA-based effector molecule” or “TREM,” as that term is used herein, refers to an RNA molecule comprising a structure or property from (a)-(v) below, and which is a recombinant TREM, a synthetic TREM, or a TREM expressed from a heterologous cell. The TREMs described in the present invention are synthetic molecules and are made, e.g., in a cell free reaction, e.g., in a solid state or liquid phase synthetic reaction. TREMs are chemically distinct, e.g., in terms of primary sequence, type or location of modifications from the endogenous tRNA molecules made in cells, e.g., in mammalian cells, e.g., in human cells. A TREM can have a plurality (e.g., 2, 3, 4, 5, 6, 7, 8, 9) of the structures and functions of (a)-(v).

In an embodiment, a TREM is non-native, as evaluated by structure or the way in which it was made.

In an embodiment, a TREM comprises one or more of the following structures or properties:
(a′) an optional linker region of a consensus sequence provided in the “Consensus Sequence” section, e.g., a Linker 1 region;
(a) an amino acid attachment domain that binds an amino acid, e.g., an acceptor stem domain (AStD), wherein an AStD comprises sufficient RNA sequence to mediate, e.g., when present in an otherwise wildtype tRNA, acceptance of an amino acid, e.g., its cognate amino acid or a non-cognate amino acid, and transfer of the amino acid (AA) in the initiation or elongation of a polypeptide chain. Typically, the AStD comprises a 3′-end adenosine (CCA) for acceptor stem charging which is part of synthetase recognition. In an embodiment the AStD has at least 75, 80, 85, 85, 90, 95, or 100% identity with a naturally occurring AStD, e.g., an AStD encoded by a nucleic acid in Table 1. In an embodiment, the TREM can comprise a fragment or analog of an AStD, e.g., an AStD encoded by a nucleic acid in Table 1, which fragment in embodiments has AStD activity and in other embodiments does not have AStD activity. (One of ordinary skill can determine the relevant corresponding sequence for any of the domains, stems, loops, or other sequence features mentioned herein from a sequence encoded by a nucleic acid in Table 1. E.g . . . one of ordinary skill can determine the sequence which corresponds to an AStD from a tRNA sequence encoded by a nucleic acid in Table 1.)

In an embodiment the AStD falls under the corresponding sequence of a consensus sequence provided in the “Consensus Sequence” section, or differs from the consensus sequence by no more than 1, 2, 5, or 10 positions;

In an embodiment, the AStD comprises residues R₁-R₂-R₃-R₄-R₅-R₆-R₇ and residues R₆₅-R₆₆-R₆₇-R₆₈-R₆₉-R₇₀-R₇₁ of Formula I zzz, wherein ZZZ indicates any of the twenty amino acids;

In an embodiment, the AStD comprises residues R₁-R₂-R₃-R₄-R₅-R₆-R₇ and residues R₆₅-R₆₆-R₆₇-R₆₈-R₆₉-R₇₀-R₇₁ of Formula II zzz, wherein ZZZ indicates any of the twenty amino acids;

In an embodiment, the AStD comprises residues R₁-R₂-R₃-R₄-R₅-R₆-R₇ and residues R₆₅-R₆₆-R₆₇-R₆₈-R₆₉-R₇₀-R₇₁ of Formula III zzz, wherein ZZZ indicates any of the twenty amino acids;
(a′-1) a linker comprising residues R₈-R₉ of a consensus sequence provided in the “Consensus Sequence” section, e.g., a Linker 2 region;
(b) a dihydrouridine hairpin domain (DHD), wherein a DHD comprises sufficient RNA sequence to mediate, e.g., when present in an otherwise wildtype tRNA, recognition of aminoacyl-tRNA synthetase, e.g., acts as a recognition site for aminoacyl-tRNA synthetase for amino acid charging of the TREM. In embodiments, a DHD mediates the stabilization of the TREM's tertiary structure. In an embodiment the DHD has at least 75, 80, 85, 85, 90, 95, or 100% identity with a naturally occurring DHD, e.g., a DHD encoded by a nucleic acid in Table 1. In an embodiment, the TREM can comprise a fragment or analog of a DHD, e.g., a DHD encoded by a nucleic acid in Table 1, which fragment in embodiments has DHD activity and in other embodiments does not have DHD activity.

In an embodiment the DHD falls under the corresponding sequence of a consensus sequence provided in the “Consensus Sequence” section, or differs from the consensus sequence by no more than 1, 2, 5, or 10 positions;

In an embodiment, the DHD comprises residues R₁₀-R₁₁-R₁₂-R₁₃-R₁₄ R₁₅-R₁₆-R₁₇-R₁₈-R₁₉-R₂₀-R₂₁-R₂₂-R₂₃-R₂₄-R₂₅-R₂₆-R₂₇-R₂₈ of Formula I zzz, wherein ZZZ indicates any of the twenty amino acids;

In an embodiment, the DHD comprises residues R₁₀-R₁₁-R₁₂-R₁₃-R₁₄ R₁₅-R₁₆-R₁₇-R₁₈-R₁₉-R₂₀-R₂₁-R₂₂-R₂₃-R₂₄-R₂₅-R₂₆-R₂₇-R₂₈ of Formula II zzz, wherein ZZZ indicates any of the twenty amino acids;

In an embodiment, the DHD comprises residues R₁₀-R₁₁-R₁₂-R₁₃-R₁₄ R₁₅-R₁₆-R₁₇-R₁₈-R₁₉-R₂₀-R₂₁-R₂₂-R₂₃-R₂₄-R₂₅-R₂₆-R₂₇-R₂₈ of Formula III zzz, wherein ZZZ indicates any of the twenty amino acids;
(b′-1) a linker comprising residue R₂₉ of a consensus sequence provided in the “Consensus Sequence” section, e.g., a Linker 3 region;
(c) an anticodon that binds a respective codon in an mRNA, e.g., an anticodon hairpin domain (ACHD), wherein an ACHD comprises sufficient sequence, e.g., an anticodon triplet, to mediate, e.g., when present in an otherwise wildtype tRNA, pairing (with or without wobble) with a codon; In an embodiment the ACHD has at least 75, 80, 85, 85, 90, 95, or 100% identity with a naturally occurring ACHD, e.g., an ACHD encoded by a nucleic acid in Table 1. In an embodiment, the TREM can comprise a fragment or analog of an ACHD, e.g., an ACHD encoded by a nucleic acid in Table 1, which fragment in embodiments has ACHD activity and in other embodiments does not have ACHD activity.

In an embodiment the ACHD falls under the corresponding sequence of a consensus sequence provided in the “Consensus Sequence” section, or differs from the consensus sequence by no more than 1, 2, 5, or 10 positions;

In an embodiment, the ACHD comprises residues-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ of Formula I zzz, wherein ZZZ indicates any of the twenty amino acids;

In an embodiment, the ACHD comprises residues-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ of Formula II zzz, wherein ZZZ indicates any of the twenty amino acids;

In an embodiment, the ACHD comprises residues-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ of Formula III zzz, wherein ZZZ indicates any of the twenty amino acids;
(d) a variable loop domain (VLD), wherein a VLD comprises sufficient RNA sequence to mediate, e.g., when present in an otherwise wildtype tRNA, recognition of aminoacyl-tRNA synthetase, e.g., acts as a recognition site for aminoacyl-tRNA synthetase for amino acid charging of the TREM. In embodiments, a VLD mediates the stabilization of the TREM's tertiary structure. In an embodiment, a VLD modulates, e.g., increases, the specificity of the TREM, e.g., for its cognate amino acid, e.g., the VLD modulates the TREM's cognate adaptor function. In an embodiment the VLD has at least 75, 80, 85, 85, 90, 95, or 100% identity with a naturally occurring VLD, e.g., a VLD encoded by a nucleic acid in Table 1. In an embodiment, the TREM can comprise a fragment or analog of a VLD, e.g., a VLD encoded by a nucleic acid in Table 1, which fragment in embodiments has VLD activity and in other embodiments does not have VLD activity.

In an embodiment the VLD falls under the corresponding sequence of a consensus sequence provided in the “Consensus Sequence” section.

In an embodiment, the VLD comprises residue-[R₄₇]ₓ of a consensus sequence provided in the “Consensus Sequence” section, wherein x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271);
(e) a thymine hairpin domain (THD), wherein a THD comprises sufficient RNA sequence, to mediate, e.g., when present in an otherwise wildtype tRNA, recognition of the ribosome, e.g., acts as a recognition site for the ribosome to form a TREM-ribosome complex during translation. In an embodiment the THD has at least 75, 80, 85, 85, 90, 95, or 100% identity with a naturally occurring THD, e.g., a THD encoded by a nucleic acid in Table 1. In an embodiment, the TREM can comprise a fragment or analog of a THD, e.g., a THD encoded by a nucleic acid in Table 1, which fragment in embodiments has THD activity and in other embodiments does not have THD activity.

In an embodiment the THD falls under the corresponding sequence of a consensus sequence provided in the “Consensus Sequence” section, or differs from the consensus sequence by no more than 1, 2, 5, or 10 positions;

In an embodiment, the THD comprises residues-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ of Formula I zzz, wherein ZZZ indicates any of the twenty amino acids;

In an embodiment, the THD comprises residues-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ of Formula II zzz, wherein ZZZ indicates any of the twenty amino acids;

In an embodiment, the THD comprises residues-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ of Formula III zzz, wherein ZZZ indicates any of the twenty amino acids;
(e′1) a linker comprising residue R₇₂ of a consensus sequence provided in the “Consensus Sequence” section, e.g., a Linker 4 region;
(f) under physiological conditions, it comprises a stem structure and one or a plurality of loop structures, e.g., 1, 2, or 3 loops. A loop can comprise a domain described herein, e.g., a domain selected from (a)-(e). A loop can comprise one or a plurality of domains. In an embodiment, a stem or loop structure has at least 75, 80, 85, 85, 90, 95, or 100% identity with a naturally occurring stem or loop structure, e.g., a stem or loop structure encoded by a nucleic acid in Table 1. In an embodiment, the TREM can comprise a fragment or analog of a stem or loop structure, e.g., a stem or loop structure encoded by a nucleic acid in Table 1, which fragment in embodiments has activity of a stem or loop structure, and in other embodiments does not have activity of a stem or loop structure;
(g) a tertiary structure, e.g., an L-shaped tertiary structure;
(h) adaptor function, i.e., the TREM mediates acceptance of an amino acid, e.g., its cognate amino acid and transfer of the AA in the initiation or elongation of a polypeptide chain;
(i) cognate adaptor function wherein the TREM mediates acceptance and incorporation of an amino acid (e.g., cognate amino acid) associated in nature with the anti-codon of the TREM to initiate or elongate a polypeptide chain;
(j) non-cognate adaptor function, wherein the TREM mediates acceptance and incorporation of an amino acid (e.g., non-cognate amino acid) other than the amino acid associated in nature with the anti-codon of the TREM in the initiation or elongation of a polypeptide chain;
(k) a regulatory function, e.g., an epigenetic function (e.g., gene silencing function or signaling pathway modulation function), cell fate modulation function, mRNA stability modulation function, protein stability modulation function, protein transduction modulation function, or protein compartmentalization function;
(l) a structure which allows for ribosome binding;
(m) a post-transcriptional modification, e.g., a naturally occurring post-trasncriptional
modification;
(n) the ability to inhibit a functional property of a tRNA, e.g., any of properties (h)-(k)
possessed by a tRNA;
(o) the ability to modulate cell fate;
(p) the ability to modulate ribosome occupancy;
(q) the ability to modulate protein translation;
(r) the ability to modulate mRNA stability;
(s) the ability to modulate protein folding and structure;
(t) the ability to modulate protein transduction or compartmentalization;
(u) the ability to modulate protein stability; or
(v) the ability to modulate a signaling pathway, e.g., a cellular signaling pathway.

In an embodiment, a TREM comprises a full-length tRNA molecule or a fragment thereof.

In an embodiment, a TREM comprises the following properties: (a)-(e).

In an embodiment, a TREM comprises the following properties: (a) and (c).

In an embodiment, a TREM comprises the following properties: (a), (c) and (h).

In an embodiment, a TREM comprises the following properties: (a), (c), (h) and (b).

In an embodiment, a TREM comprises the following properties: (a), (c), (h) and (e).

In an embodiment, a TREM comprises the following properties: (a), (c), (h), (b) and (e).

In an embodiment, a TREM comprises the following properties: (a), (c), (h), (b), (e) and (g).

In an embodiment, a TREM comprises the following properties: (a), (c), (h) and (m).

In an embodiment, a TREM comprises the following properties: (a), (c), (h), (m), and (g).

In an embodiment, a TREM comprises the following properties: (a), (c), (h), (m) and (b).

In an embodiment, a TREM comprises the following properties: (a), (c), (h), (m) and (e).

In an embodiment, a TREM comprises the following properties: (a), (c), (h), (m), (g), (b) and (e).

In an embodiment, a TREM comprises the following properties: (a), (c), (h), (m), (g), (b), (e) and (q).

In an embodiment, a TREM comprises:
(i) an amino acid attachment domain that binds an amino acid (e.g., an AStD, as described in (a) herein; and
(ii) an anticodon that binds a respective codon in an mRNA (e.g., an ACHD, as described in (c) herein).

In an embodiment the TREM comprises a flexible RNA linker which provides for covalent linkage of (i) to (ii).

In an embodiment, the TREM mediates protein translation.

In an embodiment a TREM comprises a linker, e.g., an RNA linker, e.g., a flexible RNA linker, which provides for covalent linkage between a first and a second structure or domain. In an embodiment, an RNA linker comprises at least 1, 2, 3, 4, 5, 6, 7, 8, 9, 10, 11, 12, 13, 14 or 15 ribonucleotides. A TREM can comprise one or a plurality of linkers, e.g., in embodiments a TREM comprising (a), (b), (c), (d) and (e) can have a first linker between a first and second domain, and a second linker between a third domain and another domain.

In an embodiment, the TREM comprises a sequence of Formula A: [L1]-[ASt Domain1]-[L2]-[DH Domain]-[L3]-[ACH Domain]-[VL Domain]-[TH Domain]-[L4]-[ASt Domain2].

In an embodiment, a TREM comprises an RNA sequence at least 60, 65, 70, 75, 80, 85, 90, 95, 96, 97, 98 or 99% identical with, or which differs by no more than 1, 2, 3, 4, 5, 10, 15, 20, 25, or 30 ribonucleotides from, an RNA sequence encoded by a DNA sequence listed in Table 1, or a fragment or functional fragment thereof. In an embodiment, a TREM comprises an RNA sequence encoded by a DNA sequence listed in Table 1, or a fragment or functional fragment thereof. In an embodiment, a TREM comprises an RNA sequence encoded by a DNA sequence at least 60, 65, 70, 75, 80, 85, 90, 95, 96, 97, 98 or 99% identical with a DNA sequence listed in Table 1, or a fragment or functional fragment thereof. In an embodiment, a TREM comprises a TREM domain, e.g., a domain described herein, comprising at least 60, 65, 70, 75, 80, 85, 90, 95, 96, 97, 98, or 99% identical with, or which differs by no more than 1, 2, 3, 4, 5, 10, or 15, ribonucleotides from, an RNA encoded by a DNA sequence listed in Table 1, or a fragment or a functional fragment thereof. In an embodiment, a TREM comprises a TREM domain, e.g., a domain described herein, comprising an RNA sequence encoded by DNA sequence listed in Table 1, or a fragment or functional fragment thereof. In an embodiment, a TREM comprises a TREM domain, e.g., a domain described herein, comprising an RNA sequence encoded by DNA sequence at least 60, 65, 70, 75, 80, 85, 90, 95, 96, 97, 98 or 99% identical with a DNA sequence listed in Table 1, or a fragment or functional fragment thereof.

In an embodiment, a TREM is 76-90 nucleotides in length. In embodiments, a TREM or a fragment or functional fragment thereof is between 10-90 nucleotides, between 10-80 nucleotides, between 10-70 nucleotides, between 10-60 nucleotides, between 10-50 nucleotides, between 10-40 nucleotides, between 10-30 nucleotides, between 10-20 nucleotides, between 20-90 nucleotides, between 20-80 nucleotides, 20-70 nucleotides, between 20-60 nucleotides, between 20-50 nucleotides, between 20-40 nucleotides, between 30-90 nucleotides, between 30-80 nucleotides, between 30-70 nucleotides, between 30-60 nucleotides, or between 30-50 nucleotides.

In an embodiment, a TREM is aminoacylated, e.g., charged, with an amino acid by an aminoacyl tRNA synthetase.

In an embodiment, a TREM is not charged with an amino acid, e.g., an uncharged TREM (uTREM).

In an embodiment, a TREM comprises less than a full length tRNA. In embodiments, a TREM can correspond to a naturally occurring fragment of a tRNA, or to a non-naturally occurring fragment. Exemplary fragments include: TREM halves (e.g., from a cleavage in the ACHD, e.g., in the anticodon sequence, e.g., 5′halves or 3′ halves); a 5′ fragment (e.g., a fragment comprising the 5′ end, e.g., from a cleavage in a DHD or the ACHD); a 3′ fragment (e.g., a fragment comprising the 3′ end, e.g., from a cleavage in the THD); or an internal fragment (e.g., from a cleavage in one or more of the ACHD, DHD or THD).

A “TREM core fragment,” as that term is used herein, refers to a portion of the sequence of Formula B: [L1]_{y}-[ASt Domain1]ₓ-[L2]_{y}-[DH Domain]_{y}-[L3]_{y}-[ACH Domain]ₓ-[VL Domain]_{y}-[TH Domain]_{y}-[L4]_{y}-[ASt Domain2]ₓ, wherein: x=1 and y=0 or 1.

A “TREM fragment,” as used herein, refers to a portion of a TREM, wherein the TREM comprises a sequence of Formula A: [L1]-[ASt Domain1]-[L2]-[DH Domain]-[L3]-[ACH Domain]-[VL Domain]-[TH Domain]-[L4]-[ASt Domain2].

A “cognate adaptor function TREM,” as that term is used herein, refers to a TREM which mediates initiation or elongation with the AA (the cognate AA) associated in nature with the anti-codon of the TREM.

“Decreased expression,” as that term is used herein, refers to a decrease in comparison to a reference, e.g., in the case where altered control region, or addition of an agent, results in a decreased expression of the subject product, it is decreased relative to an otherwise similar cell without the alteration or addition.

An “exogenous nucleic acid,” as that term is used herein, refers to a nucleic acid sequence that is not present in or differs by at least one nucleotide from the closest sequence in a reference cell, e.g., a cell into which the exogenous nucleic acid is introduced. In an embodiment, an exogenous nucleic acid comprises a nucleic acid that encodes a TREM.

An “exogenous TREM,” as that term is used herein, refers to a TREM that:
(a) differs by at least one nucleotide or one post transcriptional modification from the closest sequence tRNA in a reference cell, e.g., a cell into which the exogenous nucleic acid is introduced;
(b) has been introduced into a cell other than the cell in which it was transcribed;
(c) is present in a cell other than one in which it naturally occurs; or
(d) has an expression profile, e.g., level or distribution, that is non-wildtype, e.g., it is expressed at a higher level than wildtype. In an embodiment, the expression profile can be mediated by a change introduced into a nucleic acid that modulates expression or by addition of an agent that modulates expression of the RNA molecule. In an embodiment an exogenous TREM comprises 1, 2, 3 or 4 of properties (a)-(d).

A “GMP-grade composition,” as that term is used herein, refers to a composition in compliance with current good manufacturing practice (cGMP) guidelines, or other similar requirements. In an embodiment, a GMP-grade composition can be used as a pharmaceutical product.

As used herein, the terms “increasing” and “decreasing” refer to modulating that results in, respectively, greater or lesser amounts of function, expression, or activity of a particular metric relative to a reference. For example, subsequent to administration to a cell, tissue or subject of a TREM described herein, the amount of a marker of a metric (e.g., protein translation, mRNA stability, protein folding) as described herein may be increased or decreased by at least 5%, 10%, 15%, 20%, 25%, 30%, 35%, 40%, 45%, 50%, 55%, 60%, 65%, 70%, 75%, 80%, 85%, 90%, 95% or 98%, 2X, 3X, 5X. 10X or more relative to the amount of the marker prior to administration or relative to the effect of a negative control agent. The metric may be measured subsequent to administration at a time that the administration has had the recited effect, e.g., at least 12 hours, 24 hours, one week, one month, 3 months, or 6 months, after a treatment has begun.

“Increased expression,” as that term is used herein, refers to an increase in comparison to a reference, e.g., in the case where altered control region, or addition of an agent, results in an increased expression of the subject product, it is increased relative to an otherwise similar cell without the alteration or addition.

A “non-cognate adaptor function TREM,” as that term is used herein, refers to a TREM which mediates initiation or elongation with an AA (a non-cognate AA) other than the AA associated in nature with the anti-codon of the TREM. In an embodiment, a non-cognate adaptor function TREM is also referred to as a mischarged TREM (mTREM).

A “non-naturally occurring sequence,” as that term is used herein, refers to a sequence wherein an Adenine is replaced by a residue other than an analog of Adenine, a Cytosine is replaced by a residue other than an analog of Cytosine, a Guanine is replaced by a residue other than an analog of Guanine, and a Uracil is replaced by a residue other than an analog of Uracil. An analog refers to any possible derivative of the ribonucleotides, A, G, C or U. In an embodiment, a sequence having a derivative of any one of ribonucleotides A, G, C or U is a non-naturally occurring sequence.

A “pharmaceutical TREM composition,” as that term is used herein, refers to a TREM composition that is suitable for pharmaceutical use. Typically, a pharmaceutical TREM composition comprises a pharmaceutical excipient. In an embodiment the TREM will be the only active ingredient in the pharmaceutical TREM composition. In embodiments the pharmaceutical TREM composition is free, substantially free, or has less than a pharmaceutically acceptable amount, of host cell proteins, DNA, e.g., host cell DNA, endotoxins, and bacteria.

A “post-transcriptional processing.” as that term is used herein, with respect to a subject molecule, e.g., a TREM, RNA or tRNAs, refers to a covalent modification of the subject molecule. In an embodiment, the covalent modification occurs post-transcriptionally. In an embodiment, the covalent modification occurs co-transcriptionally. In an embodiment the modification is made in vivo, e.g., in a cell used to produce a TREM. In an embodiment the modification is made ex vivo, e.g., it is made on a TREM isolated or obtained from the cell which produced the TREM. In an embodiment, the post-transcriptional modification is selected from a post-transcriptional modification listed in Table 2.

A “synthetic TREM,” as that term is used herein, refers to a TREM which was synthesized other than in or by a cell having an endogenous nucleic acid encoding the TREM, e.g., a synthetic TREM is synthetized by cell-free solid phase synthesis. A synthetic TREM can have the same, or a different, sequence, or tertiary structure, as a native tRNA.

A “recombinant TREM,” as that term is used herein, refers to a TREM that was expressed in a cell modified by human intervention, having a modification that mediates the production of the TREM, e.g., the cell comprises an exogenous sequence encoding the TREM, or a modification that mediates expression, e.g., transcriptional expression or post-transcriptional modification, of the TREM. A recombinant TREM can have the same, or a different, sequence, set of post-transcriptional modifications, or tertiary structure, as a reference tRNA, e.g., a native tRNA.

A “tRNA”, as that term is used herein, refers to a naturally occurring transfer ribonucleic acid in its native state.

A “TREM composition,” as that term is used herein, refers to a composition comprising a plurality of TREMs, a plurality of TREM core fragments and/or a plurality of TREM fragments. A TREM composition can comprise one or more species of TREMs, TREM core fragments or TREM fragments. In an embodiment, the composition comprises only a single species of TREM, TREM core fragment or TREM fragment. In an embodiment, the TREM composition comprises a first TREM, TREM core fragment or TREM fragment species; and a second TREM, TREM core fragment or TREM fragment species. In an embodiment, the TREM composition comprises X TREM, TREM core fragment or TREM fragment species, wherein X=2, 3, 4, 5, 6, 7, 8, 9, or 10. In an embodiment, the TREM, TREM core fragment or TREM fragment has at least 70, 75, 80, 85, 90, or 95, or has 100%, identity with a sequence encoded by a nucleic acid in Table 1. A TREM composition can comprise one or more species of TREMs, TREM core fragments or TREM fragments. In an embodiment, the TREM composition is at least 10, 20, 30, 40, 50, 60, 70, 80, 90, 95 or 99% dry weight TREMs (for a liquid composition dry weight refers to the weight after removal of substantially all liquid, e.g., after lyophilization). In an embodiment, the composition is a liquid. In an embodiment, the composition is dry, e.g., a lyophilized material. In an embodiment, the composition is a frozen composition. In an embodiment, the composition is sterile. In an embodiment, the composition comprises at least 0.5 g. 1.0 g. 5.0 g, 10 g, 15 g, 25 g, 50 g, 100 g, 200 g, 400 g, or 500 g (e.g., as determined by dry weight) of TREM.

In an embodiment, at least X % of the TREMs in a TREM composition has a non-naturally occurring modification at a selected position, and X is 80, 90, 95, 96, 97, 98, 99, or 99.5.

In an embodiment, at least X % of the TREMs in a TREM composition has a non-naturally occurring modification at a first position and a non-naturally occurring modification at a second position, and X, independently, is 80, 90, 95, 96, 97, 98, 99, or 99.5. In embodiments, the modification at the first and second position is the same. In embodiments, the modification at the first and second position are different. In embodiments, the nucleiotide at the first and second position is the same, e.g., both are adenine. In embodiments, the nucleiotide at the first and second position are different, e.g., one is adenine and one is thymine.

In an embodiment, at least X % of the TREMs in a TREM composition has a non-naturally occurring modification at a first position and less than Y % have a non-naturally occurring modification at a second position, wherein X is 80, 90, 95, 96, 97, 98, 99, or 99.5 and Y is 20, 20, 5, 2, 1 . . . 1, or 0.01. In embodiments, the nucleotide at the first and second position is the same, e.g., both are adenine. In embodiments the nucleotide at the first and second position are different, e.g., one is adenine and one is thymine.

### TREM, TREM Core Fragment and TREM Fragment

A “tRNA-based effector molecule” or “TREM” refers to an RNA molecule comprising one or more of the properties described herein. A TREM can comprise a non-naturally occurring modification, e.g., as provided in Tables 4, 5, 6 or 7.

In an embodiment, a TREM includes a TREM comprising a sequence of Formula A; a TREM core fragment comprising a sequence of Formula B; or a TREM fragment comprising a portion of a TREM which TREM comprises a sequence of Formula A.

In an embodiment, a TREM comprises a sequence of Formula A: [L1]-[ASt Domain1]-[L2]-[DH Domain]-[L3]-[ACH Domain]-[VL Domain]-[TH Domain]-[L4]-[ASt Domain2]. In an embodiment. [VL Domain] is optional. In an embodiment, [L1] is optional.

In an embodiment, a TREM core fragment comprises a sequence of Formula B: [L1]_{y}-[ASt Domain1]ₓ-[L2]_{y}-[DH Domain]_{y}-[L3]_{y}-[ACH Domain]ₓ-[VL Domain]_{y}-[TH Domain]_{y}-[L4]_{y}-[ASt Domain2]ₓ, wherein: x=1 and y=0 or 1. In an embodiment, y=0. In an embodiment, y=1.;

In an embodiment, a TREM fragment comprises a portion of a TREM, wherein the TREM comprises a sequence of Formula A: [L1]-[ASt Domain1]-[L2]-[DH Domain]-[L3]-[ACH Domain]-[VL Domain]-[TH Domain]-[L4]-[ASt Domain2], and wherein the TREM fragment comprises: one, two, three or all or any combination of the following: a TREM half (e.g., from a cleavage in the ACH Domain, e.g., in the anticodon sequence, e.g., a 5′half or a 3′ half); a 5′ fragment (e.g., a fragment comprising the 5′ end, e.g., from a cleavage in a DH Domain or the ACH Domain); a 3′ fragment (e.g., a fragment comprising the 3′ end, e.g., from a cleavage in the TH Domain); or an internal fragment (e.g., from a cleavage in any one of the ACH Domain, DH Domain or TH Domain). Exemplary TREM fragments include TREM halves (e.g., from a cleavage in the ACHD, e.g., 5′TREM halves or 3′ TREM halves), a 5′ fragment (e.g., a fragment comprising the 5′ end, e.g., from a cleavage in a DHD or the ACHD), a 3′ fragment (e.g., a fragment comprising the 3′ end of a TREM, e.g., from a cleavage in the THD), or an internal fragment (e.g., from a cleavage in one or more of the ACHD, DHD or THD).

In an embodiment, a TREM, a TREM core fragment or a TREM fragment can be charged with an amino acid (e.g., a cognate amino acid); charged with a non-cognate amino acid (e.g., a mischarged TREM (mTREM)); or not charged with an amino acid (e.g., an uncharged TREM (uTREM)). In an embodiment, a TREM, a TREM core fragment or a TREM fragment can be charged with an amino acid selected from alanine, arginine, asparagine, aspartate, cysteine, glutamine, glutamate, glycine, histidine, isoleucine, methionine, leucine, lysine, phenylalanine, proline, serine, threonine, tryptophan, tyrosine, or valine.

In some embodiments, a non-extended anticodon is an anticodon of no more than three nucleotides. In an embodiment, a non-extended codon pairs with no more than three codon nucleotides on a nucleic acid being translated.

In an embodiment, the TREM, TREM core fragment or TREM fragment is a cognate TREM. In an embodiment, the TREM, TREM core fragment or TREM fragment is a non-cognate TREM. In an embodiment, the TREM, TREM core fragment or TREM fragment recognizes a codon provided in Table 2 or Table 3.

TABLE 2

AAA
AAC
AAG
AAU
ACA
ACC
ACG
ACU
AGA
AGC
AGG
AGU
AUA
AUC
AUG
AUU
CAA
CAC
CAG
CAU
CCA
CCC
CCG
CCU
CGA
CGC
CGG
CGU
CUA
CUC
CUG
CUU
GAA
GAC
GAG
GAU
GCA
GCC
GCG
GCU
GGA
GGC
GGG
GGU
GUA
GUC
GUG
GUU
UAA
UAC
UAG
UAU
UCA
UCC
UCG
UCU
UGA
UGC
UGG
UGU
UUA
UUC
UUG
UUU

TABLE 3
Amino acids and corresponding codons

| Amino Acid | mRNA codons |
|---|---|
| Alanine | GCU, GCC, GCA, GCG |
| Arginine | CGU, CGC, CGA, CGG, AGA, AGG |
| Asparagine | AAU, AAC |
| Aspartate | GAU, GAC |
| Cysteine | UGU, UGC |
| Glutamate | GAA, GAG |
| Glutamine | CAA, CAG |
| Glycine | GGU, GGC, GGA, GGG |
| Histidine | CAU, CAC |
| Isoleucine | AUU, AUC, AUA |
| Leucine | UUA, UUG, CUU, CUC, CUA, CUG |
| Lysine | AAA, AAG |
| Methionine | AUG |
| Phenylalanine | UUU, UUC |
| Proline | CCU, CCC, CCA, CCG |
| Serine | UCU, UCC, UCA, UCG, AGU, AGC |
| Stop | UAA, UAG, UGA |
| Threonine | ACU, ACC, ACA, ACG |
| Tryptophan | UGG |
| Tyrosine | UAU, UAC |
| Valine | GUU, GUC, GUA, GUG |

In an embodiment, a TREM comprises a ribonucleic acid (RNA) sequence encoded by a deoxyribonucleic acid (DNA) sequence disclosed in Table 1, e.g., any one of SEQ ID NOs: 1-451 disclosed in Table 1. In an embodiment, a TREM comprises an RNA sequence at least 60%, 65%, 70%, 75%, 80%, 82%, 85%, 87%, 88%, 90%, 92%, 95%, 96%, 97%, 98%, or 99% identical to an RNA sequence encoded by a DNA sequence provided in Table 1, e.g., any one of SEQ ID NOs: 1-451 disclosed in Table 1. In an embodiment, a TREM comprises an RNA sequence encoded by a DNA sequence at least 60%, 65%, 70%, 75%, 80%, 82%, 85%, 87%, 88%, 90%, 92%, 95%, 96%, 97%, 98%, or 99% identical to a DNA sequence provided in Table 1, e.g., any one of SEQ ID NOs: 1-451 disclosed in Table 1.

In an embodiment, a TREM, a TREM core fragment, or TREM fragment comprises at least 5, 10, 15, 20, 25, or 30 consecutive nucleotides of an RNA sequence encoded by a DNA sequence disclosed in Table 1, e.g., at least 5, 10, 15, 20, 25, or 30 consecutive nucleotides of an RNA sequence encoded by any one of SEQ ID NOs: 1-451 disclosed in Table 1. In an embodiment, a TREM, a TREM core fragment, or TREM fragment comprises at least 5, 10, 15, 20, 25, or 30 consecutive nucleotides of an RNA sequence at least 60%, 65%, 70%, 75%, 80%. 82%, 85%, 87%, 88%, 90%, 92%, 95%, 96%, 97%, 98%, or 99% identical to an RNA sequence encoded by a DNA sequence provided in Table 1, e.g., any one of SEQ ID NOs: 1-451 disclosed in Table 1. In an embodiment, a TREM, a TREM core fragment, or TREM fragment comprises at least 5, 10, 15, 20, 25, or 30 consecutive nucleotides of an RNA sequence encoded by a DNA sequence at least 60%, 65%, 70%, 75%, 80%, 82%, 85%, 87%, 88%, 90%, 92%, 95%, 96%, 97%, 98%, or 99% identical to a DNA sequence provided in Table 1, e.g., any one of SEQ ID NOs: 1-451 disclosed in Table 1.

In an embodiment, a TREM core fragment or a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence encoded by a DNA sequence provided in Table 1, e.g., any one of SEQ ID NOs: 1-451 disclosed in Table 1. In an embodiment, a TREM core fragment or a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence at least 80%, 85%, 90%, 95%, 96%, 97%, 98%, or 99% identical to an RNA sequence encoded by a DNA sequence provided in Table 1, e.g., any one of SEQ ID NOs: 1-451 disclosed in Table 1. In an embodiment, a TREM core fragment or a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence encoded by a DNA sequence at least 80%, 85%, 90%, 95%, 96%, 97%, 98%, or 99% identical to a DNA sequence provided in Table 1, e.g., any one of SEQ ID NOs: 1-451 disclosed in Table 1.

In an embodiment, a TREM core fragment or a TREM fragment comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence encoded by a DNA sequence disclosed in Table 1, e.g., any one of SEQ ID NOs: 1-451 disclosed in Table 1. In an embodiment, a TREM core fragment or a TREM fragment comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence which is at least 80%, 85%, 90%, 95%, 96%, 97%, 98%, 99% or 100% identical to an RNA sequence encoded by a DNA sequence provided in Table 1, e.g., any one of SEQ ID NOs: 1-451 disclosed in Table 1. In an embodiment, a TREM core fragment or a TREM fragment comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence encoded by a DNA sequence with at least 80%, 82%, 85%, 87%, 88%, 90%, 92%, 95%, 96%, 97%, 98%, 99% or 100% identity to a DNA sequence provided in Table 1, e.g., any one of SEQ ID NOs: 1-451 disclosed in Table 1.

In an embodiment, a TREM core fragment or a TREM fragment comprises a sequence of a length of between 10-90 ribonucleotides (rnt), between 10-80 rnt, between 10-70 rnt, between 10-60 rnt, between 10-50 rnt, between 10-40 rnt, between 10-30 rnt, between 10-20 rnt, between 20-90 rnt, between 20-80 rnt, 20-70 rnt, between 20-60 rnt, between 20-50 rnt, between 20-40 rnt, between 30-90 rnt, between 30-80 rnt, between 30-70 rnt, between 30-60 rnt, or between 30-50 rnt

TABLE 1
List of tRNA Sequences

| SEQ ID | | |
|---|---|---|
| NO | tRNA name | tRNA sequence |
| 1 | Ala_AGC_chr6:28763 | GGGGGTATAGCTCAGTGGTAGAGCGCGTGCT |
| | 741-28763812 (−) | TAGCATGCACGAGGTCCTGGGTTCGATCCCC |
| 2 | Ala_AGC_chr6:26687 | GGGGAATTAGCTCAAGTGGTAGAGCGCTTGC |
| | 485-26687557 (+) | TTAGCACGCAAGAGGTAGTGGGATCGATGCC |
| 3 | Ala_AGC_chr6:26572 | GGGGAATTAGCTCAAATGGTAGAGCGCTCGC |
| | 092-26572164 (−) | TTAGCATGCGAGAGGTAGCGGGATCGATGCC |
| 4 | Ala_AGC_chr6: 26682 | GGGGAATTAGCTCAAGTGGTAGAGCGCTTGC |
| | 715-26682787 (+) | TTAGCATGCAAGAGGTAGTGGGATCGATGCC |
| 5 | Ala_AGC_chr6: 26705 | GGGGAATTAGCTCAAGCGGTAGAGCGCTTGC |
| | 606-26705678 (+) | TTAGCATGCAAGAGGTAGTGGGATCGATGCC |
| 6 | Ala_AGC_chr6: 26673 | GGGGAATTAGCTCAAGTGGTAGAGCGCTTGC |
| | 590-26673662 (+) | TTAGCATGCAAGAGGTAGTGGGATCAATGCC |
| 7 | Ala_AGC_chr14: 8944 | GGGGAATTAGCTCAAGTGGTAGAGCGCTCGC |
| | 5442-89445514 (+) | TTAGCATGCGAGAGGTAGTGGGATCGATGCC |
| 8 | Ala_AGC_chr6: 58196 | GGGGAATTAGCCCAAGTGGTAGAGCGCTTGC |
| | 623-58196695 (−) | TTAGCATGCAAGAGGTAGTGGGATCGATGCC |
| 9 | Ala_AGC_chr6: 28806 | GGGGGTGTAGCTCAGTGGTAGAGCGCGTGCT |
| | 221-28806292 (−) | TAGCATGCACGAGGCCCCGGGTTCAATCCCC |
| 10 | Ala_AGC_chr6: 28574 | GGGGGTGTAGCTCAGTGGTAGAGCGCGTGCT |
| | 933-28575004 (+) | TAGCATGTACGAGGTCCCGGGTTCAATCCCC |
| 11 | Ala_AGC_chr6: 28626 | GGGGATGTAGCTCAGTGGTAGAGCGCATGCT |
| | 014-28626085 (−) | TAGCATGCATGAGGTCCCGGGTTCGATCCCC |
| 12 | Ala_AGC_chr6: 28678 | GGGGGTGTAGCTCAGTGGTAGAGCGCGTGCT |
| | 366-28678437 (+) | TAGCATGCACGAGGCCCTGGGTTCAATCCCC |
| 13 | Ala_AGC_chr6: 28779 | GGGGGTATAGCTCAGCGGTAGAGCGCGTGCT |
| | 849-28779920 (−) | TAGCATGCACGAGGTCCTGGGTTCAATCCCC |
| 14 | Ala_AGC_chr6: 28687 | GGGGGTGTAGCTCAGTGGTAGAGCGCGTGCT |
| | 481-28687552 (+) | TAGCATGCACGAGGCCCCGGGTTCAATCCCT |
| 15 | Ala_AGC_chr2: 27274 | GGGGGATTAGCTCAAATGGTAGAGCGCTCGC |
| | 082-27274154 (+) | TTAGCATGCGAGAGGTAGCGGGATCGATGCC |
| 16 | Ala_AGC_chr6: 26730 | GGGGAATTAGCTCAGGCGGTAGAGCGCTCGC |
| | 737-26730809 (+) | TTAGCATGCGAGAGGTAGCGGGATCGACGCC |
| 17 | Ala_CGC_chr6: 26553 | GGGGATGTAGCTCAGTGGTAGAGCGCATGCT |
| | 731-26553802 (+) | TCGCATGTATGAGGTCCCGGGTTCGATCCCC |
| 18 | Ala_CGC_chr6: 28641 | GGGGATGTAGCTCAGTGGTAGAGCGCATGCT |
| | 613-28641684 (−) | TCGCATGTATGAGGCCCCGGGTTCGATCCCC |
| 19 | Ala_CGC_chr2: 15725 | GGGGATGTAGCTCAGTGGTAGAGCGCGCGCT |
| | 7281-157257352 (+) | TCGCATGTGTGAGGTCCCGGGTTCAATCCCC |
| 20 | Ala_CGC_chr6: 28697 | GGGGGTGTAGCTCAGTGGTAGAGCGCGTGCT |
| | 092-28697163 (+) | TCGCATGTACGAGGCCCCGGGTTCGACCCCC |
| 21 | Ala_TGC_chr6: 28757 | GGGGGTGTAGCTCAGTGGTAGAGCGCATGCT |
| | 547-28757618 (−) | TTGCATGTATGAGGTCCCGGGTTCGATCCCC |
| 22 | Ala_TGC_chr6: 28611 | GGGGATGTAGCTCAGTGGTAGAGCGCATGCT |
| | 222-28611293 (+) | TTGCATGTATGAGGTCCCGGGTTCGATCCCC |
| 23 | Ala_TGC_chr5: 18063 | GGGGATGTAGCTCAGTGGTAGAGCGCATGCT |
| | 3868-180633939 (+) | TTGCATGTATGAGGCCCCGGGTTCGATCCCC |
| 24 | Ala_TGC_chr12: 1254 | GGGGATGTAGCTCAGTGGTAGAGCGCATGCT |
| | 24512-125424583 (+) | TTGCACGTATGAGGCCCCGGGTTCAATCCCC |
| 25 | Ala_TGC_chr6: 28785 | GGGGGTGTAGCTCAGTGGTAGAGCGCATGCT |
| | 012-28785083 (−) | TTGCATGTATGAGGCCTCGGGTTCGATCCCC |
| 26 | Ala_TGC_chr6: 28726 | GGGGGTGTAGCTCAGTGGTAGAGCACATGCT |
| | 141-28726212 (−) | TTGCATGTGTGAGGCCCCGGGTTCGATCCCC |
| 27 | Ala_TGC_chr6: 28770 | GGGGGTGTAGCTCAGTGGTAGAGCGCATGCT |
| | 577-28770647 (−) | TTGCATGTATGAGGCCTCGGTTCGATCCCCG |
| 28 | Arg_ACG_chr6: 26328 | GGGCCAGTGGCGCAATGGATAACGCGTCTGA |
| | 368-26328440 (+) | CTACGGATCAGAAGATTCCAGGTTCGACTCC |
| 29 | Arg_ACG_chr3: 45730 | GGGCCAGTGGCGCAATGGATAACGCGTCTGA |
| | 491-45730563 (−) | CTACGGATCAGAAGATTCTAGGTTCGACTCC |
| 30 | Arg_CCG_chr6: 28710 | GGCCGCGTGGCCTAATGGATAAGGCGTCTGA |
| | 729-28710801 (−) | TTCCGGATCAGAAGATTGAGGGTTCGAGTCC |
| 31 | Arg_CCG_chr17: 6601 | GACCCAGTGGCCTAATGGATAAGGCATCAGC |
| | 6013-66016085 (−) | CTCCGGAGCTGGGGATTGTGGGTTCGAGTCC |
| 32 | Arg_CCT_chr17: 7303 | GCCCCAGTGGCCTAATGGATAAGGCACTGGC |
| | 0001-73030073 (+) | CTCCTAAGCCAGGGATTGTGGGTTCGAGTCC |
| 33 | Arg_CCT_chr17: 7303 | GCCCCAGTGGCCTAATGGATAAGGCACTGGC |
| | 0526-73030598 (−) | CTCCTAAGCCAGGGATTGTGGGTTCGAGTCC |
| 34 | Arg_CCT_chr16: 3202 | GCCCCGGTGGCCTAATGGATAAGGCATTGGC |
| | 901-3202973 (+) | CTCCTAAGCCAGGGATTGTGGGTTCGAGTCC |
| 35 | Arg_CCT_chr7: 13902 | GCCCCAGTGGCCTAATGGATAAGGCATTGGC |
| | 5446-139025518 (+) | CTCCTAAGCCAGGGATTGTGGGTTCGAGTCC |
| 36 | Arg_CCT_chr16: 3243 | GCCCCAGTGGCCTGATGGATAAGGTACTGGC |
| | 918-3243990 (+) | CTCCTAAGCCAGGGATTGTGGGTTCGAGTTC |
| 37 | Arg_TCG_chr15: 8987 | GGCCGCGTGGCCTAATGGATAAGGCGTCTGA |
| | 8304-89878376 (+) | CTTCGGATCAGAAGATTGCAGGTTCGAGTCC |
| 38 | Arg_TCG_chr6: 26323 | GACCACGTGGCCTAATGGATAAGGCGTCTGA |
| | 046-26323118 (+) | CTTCGGATCAGAAGATTGAGGGTTCGAATCC |
| 39 | Arg_TCG_chr17: 7303 | GACCGCGTGGCCTAATGGATAAGGCGTCTGA |
| | 1208-73031280 (+) | CTTCGGATCAGAAGATTGAGGGTTCGAGTCC |
| 40 | Arg_TCG_chr6: 26299 | GACCACGTGGCCTAATGGATAAGGCGTCTGA |
| | 905-26299977 (+) | CTTCGGATCAGAAGATTGAGGGTTCGAATCC |
| 41 | Arg_TCG_chr6: 28510 | GACCACGTGGCCTAATGGATAAGGCGTCTGA |
| | 891-28510963 (−) | CTTCGGATCAGAAGATTGAGGGTTCGAATCC |
| 42 | Arg_TCG_chr9: 11296 | GGCCGTGTGGCCTAATGGATAAGGCGTCTGA |
| | 0803-112960875 (+) | CTTCGGATCAAAAGATTGCAGGTTTGAGTTC |
| 43 | Arg_TCT_chr1: 94313 | GGCTCCGTGGCGCAATGGATAGCGCATTGGA |
| | 129-94313213 (+) | CTTCTAGAGGCTGAAGGCATTCAAAGGTTCC |
| 44 | Arg_TCT_chr17: 8024 | GGCTCTGTGGCGCAATGGATAGCGCATTGGA |
| | 243-8024330 (+) | CTTCTAGTGACGAATAGAGCAATTCAAAGGT |
| 45 | Arg_TCT_chr9: 13110 | GGCTCTGTGGCGCAATGGATAGCGCATTGGA |
| | 2355-131102445 (−) | CTTCTAGCTGAGCCTAGTGTGGTCATTCAAA |
| 46 | Arg_TCT_chr11: 5931 | GGCTCTGTGGCGCAATGGATAGCGCATTGGA |
| | 8767-59318852 (+) | CTTCTAGATAGTTAGAGAAATTCAAAGGTTG |
| 47 | Arg_TCT_chr1: 15911 | GTCTCTGTGGCGCAATGGACGAGCGCGCTGG |
| | 1401-159111474 (−) | ACTTCTAATCCAGAGGTTCCGGGTTCGAGTC |
| 48 | Arg_TCT_chr6: 27529 | GGCTCTGTGGCGCAATGGATAGCGCATTGGA |
| | 963-27530049 (+) | CTTCTAGCCTAAATCAAGAGATTCAAAGGTT |
| 49 | Asn_GTT_chr1: 16151 | GTCTCTGTGGCGCAATCGGTTAGCGCGTTCG |
| | 0031-161510104 (+) | GCTGTTAACCGAAAGGTTGGTGGTTCGATCC |
| 50 | Asn_GTT_chr1: 14387 | GTCTCTGTGGCGCAATCGGCTAGCGCGTTTG |
| | 9832-143879905 (−) | GCTGTTAACTAAAAGGTTGGCGGTTCGAACC |
| 51 | Asn_GTT_chr1: 14430 | GTCTCTGTGGTGCAATCGGTTAGCGCGTTCCG |
| | 1611-144301684 (+) | CTGTTAACCGAAAGCTTGGTGGTTCGAGCCC |
| 52 | Asn_GTT_chr1: 14932 | GTCTCTGTGGCGCAATCGGCTAGCGCGTTTG |
| | 6272-149326345 (−) | GCTGTTAACTAAAAAGTTGGTGGTTCGAACA |
| 53 | Asn_GTT_chr1: 14824 | GTCTCTGTGGCGCAATCGGTTAGCGCGTTCG |
| | 8115-148248188 (+) | GCTGTTAACCGAAAGGTTGGTGGTTCGAGCC |
| 54 | Asn_GTT_chr1: 14859 | GTCTCTGTGGCGCAATCGGTTAGCGCATTCG |
| | 8314-148598387 (−) | GCTGTTAACCGAAAGGTTGGTGGTTCGAGCC |
| 55 | Asn_GTT_chr1: 17216 | GTCTCTGTGGCGCAATCGGTTAGCGCGTTCG |
| | 172-17216245 (+) | GCTGTTAACCGAAAGATTGGTGGTTCGAGCC |
| 56 | Asn_GTT_chr1: 16847 | GTCTCTGTGGCGCAATCGGTTAGCGCGTTCG |
| | 080-16847153 (−) | GCTGTTAACTGAAAGGTTGGTGGTTCGAGCC |
| 57 | Asn_GTT_chr1: 14923 | GTCTCTGTGGCGCAATGGGTTAGCGCGTTCG |
| | 0570-149230643 (−) | GCTGTTAACCGAAAGGTTGGTGGTTCGAGCC |
| 58 | Asn_GTT_chr1: 14800 | GTCTCTGTGGCGTAGTCGGTTAGCGCGTTCG |
| | 0805-148000878 (+) | GCTGTTAACCGAAAAGTTGGTGGTTCGAGCC |
| 59 | Asn_GTT_chr1: 14971 | GTCTCTGTGGCGCAATCGGCTAGCGCGTTTG |
| | 1798-149711871 (−) | GCTGTTAACTAAAAGGTTGGTGGTTCGAACC |
| 60 | Asn_GTT_chr1: 14597 | GTCTCTGTGGCGCAATCGGTTAGCGCGTTCG |
| | 9034-145979107 (−) | GCTGTTAACTGAAAGGTTAGTGGTTCGAGCC |
| 61 | Asp_GTC_chr12: 9889 | TCCTCGTTAGTATAGTGGTTAGTATCCCCGCC |
| | 7281-98897352 (+) | TGTCACGCGGGAGACCGGGGTTCAATTCCCC |
| 62 | Asp_GTC_chr1: 16141 | TCCTCGTTAGTATAGTGGTGAGTATCCCCGCC |
| | 0615-161410686 (−) | TGTCACGCGGGAGACCGGGGTTCGATTCCCC |
| 63 | Asp_GTC_chr6: 27551 | TCCTCGTTAGTATAGTGGTGAGTGTCCCCGTC |
| | 236-27551307 (−) | TGTCACGCGGGAGACCGGGGTTCGATTCCCC |
| 64 | Cys_GCA_chr7: 14900 | GGGGGCATAGCTCAGTGGTAGAGCATTTGAC |
| | 7281-149007352 (+) | TGCAGATCAAGAGGTCCCTGGTTCAAATCCA |
| 65 | Cys_GCA_chr7: 14907 | GGGGGTATAGCTCAGGGGTAGAGCATTTGAC |
| | 4601-149074672 (−) | TGCAGATCAAGAGGTCCCTGGTTCAAATCCA |
| 66 | Cys_GCA_chr7: 14911 | GGGGGTATAGCTTAGCGGTAGAGCATTTGAC |
| | 2229-149112300 (−) | TGCAGATCAAGAGGTCCCCGGTTCAAATCCG |
| 67 | Cys_GCA_chr7: 14934 | GGGGGTATAGCTTAGGGGTAGAGCATTTGAC |
| | 4046-149344117 (−) | TGCAGATCAAAAGGTCCCTGGTTCAAATCCA |
| 68 | Cys_GCA_chr7: 14905 | GGGGGTATAGCTCAGGGGTAGAGCATTTGAC |
| | 2766-149052837 (−) | TGCAGATCAAGAGGTCCCCAGTTCAAATCTG |
| 69 | Cys_GCA_chr17: 3701 | GGGGGTATAGCTCAGGGGTAGAGCATTTGAC |
| | 7937-37018008 (−) | TGCAGATCAAGAAGTCCCCGGTTCAAATCCG |
| 70 | Cys_GCA_chr7: 14928 | GGGGGTATAGCTCAGGGGTAGAGCATTTGAC |
| | 1816-149281887 (+) | TGCAGATCAAGAGGTCTCTGGTTCAAATCCA |
| 71 | Cys_GCA_chr7: 14924 | GGGGGTATAGCTCAGGGGTAGAGCACTTGAC |
| | 3631-149243702 (+) | TGCAGATCAAGAAGTCCTTGGTTCAAATCCA |
| 72 | Cys_GCA_chr7: 14938 | GGGGATATAGCTCAGGGGTAGAGCATTTGAC |
| | 8272-149388343 (−) | TGCAGATCAAGAGGTCCCCGGTTCAAATCCG |
| 73 | Cys_GCA_chr7: 14907 | GGGGGTATAGTTCAGGGGTAGAGCATTTGAC |
| | 2850-149072921 (−) | TGCAGATCAAGAGGTCCCTGGTTCAAATCCA |
| 74 | Cys_GCA_chr7: 14931 | GGGGGTATAGCTCAGGGGTAGAGCATTTGAC |
| | 0156-149310227 (−) | TGCAAATCAAGAGGTCCCTGATTCAAATCCA |
| 75 | Cys_GCA_chr4: 12443 | GGGGGTATAGCTCAGTGGTAGAGCATTTGAC |
| | 0005-124430076 (−) | TGCAGATCAAGAGGTCCCCGGTTCAAATCCG |
| 76 | Cys_GCA_chr7: 14929 | GGGCGTATAGCTCAGGGGTAGAGCATTTGAC |
| | 5046-149295117 (+) | TGCAGATCAAGAGGTCCCCAGTTCAAATCTG |
| 77 | Cys_GCA_chr7: 14936 | GGGGGTATAGCTCACAGGTAGAGCATTTGAC |
| | 1915-149361986 (+) | TGCAGATCAAGAGGTCCCCGGTTCAAATCTG |
| 78 | Cys_GCA_chr7: 14925 | GGGCGTATAGCTCAGGGGTAGAGCATTTGAC |
| | 3802-149253871 (+) | TGCAGATCAAGAGGTCCCCAGTTCAAATCTG |
| 79 | Cys_GCA_chr7: 14929 | GGGGGTATAGCTCACAGGTAGAGCATTTGAC |
| | 2305-149292376 (−) | TGCAGATCAAGAGGTCCCCGGTTCAAATCCG |
| 80 | Cys_GCA_chr7: 14928 | GGGGGTATAGCTCAGGGGTAGAGCACTTGAC |
| | 6164-149286235 (−) | TGCAGATCAAGAGGTCCCTGGTTCAAATCCA |
| 81 | Cys_GCA_chr17: 3702 | GGGGGTATAGCTCAGTGGTAGAGCATTTGAC |
| | 5545-37025616 (−) | TGCAGATCAAGAGGTCCCTGGTTCAAATCCG |
| 82 | Cys_GCA_chr15: 8003 | GGGGGTATAGCTCAGTGGGTAGAGCATTTGA |
| | 6997-80037069 (+) | CTGCAGATCAAGAGGTCCCCGGTTCAAATCC |
| 83 | Cys_GCA_chr3: 13194 | GGGGGTGTAGCTCAGTGGTAGAGCATTTGAC |
| | 7944-131948015 (−) | TGCAGATCAAGAGGTCCCTGGTTCAAATCCA |
| 84 | Cys_GCA_chr1: 93981 | GGGGGTATAGCTCAGGTGGTAGAGCATTTGA |
| | 834-93981906 (−) | CTGCAGATCAAGAGGTCCCCGGTTCAAATCC |
| 85 | Cys_GCA_chr14: 7342 | GGGGGTATAGCTCAGGGGTAGAGCATTTGAC |
| | 9679-73429750 (+) | TGCAGATCAAGAGGTCCCCGGTTCAAATCCG |
| 86 | Cys_GCA_chr3: 13195 | GGGGGTATAGCTCAGGGGTAGAGCATTTGAC |
| | 0642-131950713 (−) | TGCAGATCAAGAGGTCCCTGGTTCAAATCCA |
| 87 | Gln_CTG_chr6: 18836 | GGTTCCATGGTGTAATGGTTAGCACTCTGGA |
| | 402-18836473 (+) | CTCTGAATCCAGCGATCCGAGTTCAAATCTC |
| 88 | Gln_CTG_chr6: 27515 | GGTTCCATGGTGTAATGGTTAGCACTCTGGA |
| | 531-27515602 (−) | CTCTGAATCCAGCGATCCGAGTTCAAGTCTC |
| 89 | Gln_CTG_chr1: 14596 | GGTTCCATGGTGTAATGGTGAGCACTCTGGA |
| | 3304-145963375 (+) | CTCTGAATCCAGCGATCCGAGTTCGAGTCTC |
| 90 | Gln_CTG_chr1: 14773 | GGTTCCATGGTGTAATGGTAAGCACTCTGGA |
| | 7382-147737453 (−) | CTCTGAATCCAGCGATCCGAGTTCGAGTCTC |
| 91 | Gln_CTG_chr6: 27263 | GGTTCCATGGTGTAATGGTTAGCACTCTGGA |
| | 212-27263283 (+) | CTCTGAATCCGGTAATCCGAGTTCAAATCTC |
| 92 | Gln_CTG_chr6: 27759 | GGCCCCATGGTGTAATGGTCAGCACTCTGGA |
| | 135-27759206 (−) | CTCTGAATCCAGCGATCCGAGTTCAAATCTC |
| 93 | Gln_CTG_chr1: 14780 | GGTTCCATGGTGTAATGGTAAGCACTCTGGA |
| | 0937-147801008 (+) | CTCTGAATCCAGCCATCTGAGTTCGAGTCTCT |
| 94 | Gln_TTG_chr17: 4726 | GGTCCCATGGTGTAATGGTTAGCACTCTGGA |
| | 9890-47269961 (+) | CTTTGAATCCAGCGATCCGAGTTCAAATCTC |
| 95 | Gln_TTG_chr6: 28557 | GGTCCCATGGTGTAATGGTTAGCACTCTGGA |
| | 156-28557227 (+) | CTTTGAATCCAGCAATCCGAGTTCGAATCTC |
| 96 | Gln_TTG_chr6: 26311 | GGCCCCATGGTGTAATGGTTAGCACTCTGGA |
| | 424-26311495 (−) | CTTTGAATCCAGCGATCCGAGTTCAAATCTC |
| 97 | Gln_TTG_chr6: 14550 | GGTCCCATGGTGTAATGGTTAGCACTCTGGG |
| | 3859-145503930 (+) | CTTTGAATCCAGCAATCCGAGTTCGAATCTTG |
| 98 | Glu_CTC_chr1: 14539 | TCCCTGGTGGTCTAGTGGTTAGGATTCGGCG |
| | 9233-145399304 (−) | CTCTCACCGCCGCGGCCCGGGTTCGATTCCC |
| 99 | Glu_CTC_chr1: 24916 | TCCCTGGTGGTCTAGTGGTTAGGATTCGGCG |
| | 8447-249168518 (+) | CTCTCACCGCCGCGGCCCGGGTTCGATTCCC |
| 100 | Glu_TTC_chr2: 13109 | TCCCATATGGTCTAGCGGTTAGGATTCCTGGT |
| | 4701-131094772 (−) | TTTCACCCAGGTGGCCCGGGTTCGACTCCCG |
| 101 | Glu_TTC_chr13: 4549 | TCCCACATGGTCTAGCGGTTAGGATTCCTGGT |
| | 2062-45492133 (−) | TTTCACCCAGGCGGCCCGGGTTCGACTCCCG |
| 102 | Glu_TTC_chr1: 17199 | TCCCTGGTGGTCTAGTGGCTAGGATTCGGCG |
| | 078-17199149 (+) | CTTTCACCGCCGCGGCCCGGGTTCGATTCCCG |
| 103 | Glu_TTC_chr1: 16861 | TCCCTGGTGGTCTAGTGGCTAGGATTCGGCG |
| | 774-16861845 (−) | CTTTCACCGCCGCGGCCCGGGTTCGATTCCCG |
| 104 | Gly_CCC_chr1: 16872 | GCATTGGTGGTTCAGTGGTAGAATTCTCGCCT |
| | 434-16872504 (−) | CCCACGCGGGAGACCCGGGTTCAATTCCCGG |
| 105 | Gly_CCC_chr2: 70476 | GCGCCGCTGGTGTAGTGGTATCATGCAAGAT |
| | 123-70476193 (−) | TCCCATTCTTGCGACCCGGGTTCGATTCCCGG |
| 106 | Gly_CCC_chr17: 1976 | GCATTGGTGGTTCAATGGTAGAATTCTCGCCT |
| | 4175-19764245 (+) | CCCACGCAGGAGACCCAGGTTCGATTCCTGG |
| 107 | Gly_GCC_chr1: 16141 | GCATGGGTGGTTCAGTGGTAGAATTCTCGCC |
| | 3094-161413164 (+) | TGCCACGCGGGAGGCCCGGGTTCGATTCCCG |
| 108 | Gly_GCC_chr1: 16149 | GCATTGGTGGTTCAGTGGTAGAATTCTCGCCT |
| | 3637-161493707 (−) | GCCACGCGGGAGGCCCGGGTTCGATTCCCGG |
| 109 | Gly_GCC_chr16: 7081 | GCATTGGTGGTTCAGTGGTAGAATTCTCGCCT |
| | 2114-70812184 (−) | GCCACGCGGGAGGCCCGGGTTTGATTCCCGG |
| 110 | Gly_GCC_chr1: 16145 | GCATAGGTGGTTCAGTGGTAGAATTCTTGCC |
| | 0356-161450426 (+) | TGCCACGCAGGAGGCCCAGGTTTGATTCCTG |
| 111 | Gly_GCC_chr16: 7082 | GCATTGGTGGTTCAGTGGTAGAATTCTCGCCT |
| | 2597-70822667 (+) | GCCATGCGGGCGGCCGGGCTTCGATTCCTGG |
| 112 | Gly_TCC_chr19: 4724 | GCGTTGGTGGTATAGTGGTTAGCATAGCTGC |
| | 082-4724153 (+) | CTTCCAAGCAGTTGACCCGGGTTCGATTCCC |
| 113 | Gly_TCC_chr1: 14539 | GCGTTGGTGGTATAGTGGTGAGCATAGCTGC |
| | 7864-145397935 (−) | CTTCCAAGCAGTTGACCCGGGTTCGATTCCC |
| 114 | Gly_TCC_chr17: 8124 | GCGTTGGTGGTATAGTGGTAAGCATAGCTGC |
| | 866-8124937 (+) | CTTCCAAGCAGTTGACCCGGGTTCGATTCCC |
| 115 | Gly_TCC_chr1: 16140 | GCGTTGGTGGTATAGTGGTGAGCATAGTTGC |
| | 9961-161410032 (−) | CTTCCAAGCAGTTGACCCGGGCTCGATTCCC |
| 116 | His_GTG_chr1: 14539 | GCCGTGATCGTATAGTGGTTAGTACTCTGCGT |
| | 6881-145396952 (−) | TGTGGCCGCAGCAACCTCGGTTCGAATCCGA |
| 117 | His_GTG_chr1: 14915 | GCCATGATCGTATAGTGGTTAGTACTCTGCG |
| | 5828-149155899 (−) | CTGTGGCCGCAGCAACCTCGGTTCGAATCCG |
| 118 | Ile_AAT_chr6: 581492 | GGCCGGTTAGCTCAGTTGGTTAGAGCGTGGC |
| | 54-58149327 (+) | GCTAATAACGCCAAGGTCGCGGGTTCGATCC |
| 119 | Ile_AAT_chr6: 276559 | GGCCGGTTAGCTCAGTTGGTTAGAGCGTGGT |
| | 67-27656040 (+) | GCTAATAACGCCAAGGTCGCGGGTTCGATCC |
| 120 | Ile_AAT_chr6: 272429 | GGCTGGTTAGCTCAGTTGGTTAGAGCGTGGT |
| | 90-27243063 (−) | GCTAATAACGCCAAGGTCGCGGGTTCGATCC |
| 121 | Ile_AAT_chr17: 81303 | GGCCGGTTAGCTCAGTTGGTTAGAGCGTGGT |
| | 09-8130382 (−) | GCTAATAACGCCAAGGTCGCGGGTTCGAACC |
| 122 | Ile_AAT_chr6: 265543 | GGCCGGTTAGCTCAGTTGGTTAGAGCGTGGT |
| | 50-26554423 (+) | GCTAATAACGCCAAGGTCGCGGGTTCGATCC |
| 123 | Ile_AAT_chr6: 267452 | GGCCGGTTAGCTCAGTTGGTTAGAGCGTGGT |
| | 55-26745328 (−) | GCTAATAACGCTAAGGTCGCGGGTTCGATCC |
| 124 | Ile_AAT_chr6: 267212 | GGCCGGTTAGCTCAGTTGGTCAGAGCGTGGT |
| | 21-26721294 (−) | GCTAATAACGCCAAGGTCGCGGGTTCGATCC |
| 125 | Ile_AAT_chr6: 276363 | GGCCGGTTAGCTCAGTCGGCTAGAGCGTGGT |
| | 62-27636435 (+) | GCTAATAACGCCAAGGTCGCGGGTTCGATCC |
| 126 | Ile_AAT_chr6: 272417 | GGCTGGTTAGTTCAGTTGGTTAGAGCGTGGT |
| | 39-27241812 (+) | GCTAATAACGCCAAGGTCGTGGGTTCGATCC |
| 127 | Ile_GAT_chrX: 37564 | GGCCGGTTAGCTCAGTTGGTAAGAGCGTGGT |
| | 18-3756491 (−) | GCTGATAACACCAAGGTCGCGGGCTCGACTC |
| 128 | Ile_TAT_chr19: 39902 | GCTCCAGTGGCGCAATCGGTTAGCGCGCGGT |
| | 808-39902900 (−) | ACTTATATGACAGTGCGAGCGGAGCAATGCC |
| 129 | Ile_TAT_chr2: 430376 | GCTCCAGTGGCGCAATCGGTTAGCGCGCGGT |
| | 76-43037768 (+) | ACTTATACAGCAGTACATGCAGAGCAATGCC |
| 130 | Ile_TAT_chr6: 269881 | GCTCCAGTGGCGCAATCGGTTAGCGCGCGGT |
| | 25-26988218 (+) | ACTTATATGGCAGTATGTGTGCGAGTGATGC |
| 131 | Ile_TAT_chr6: 275992 | GCTCCAGTGGCGCAATCGGTTAGCGCGCGGT |
| | 00-27599293 (+) | ACTTATACAACAGTATATGTGCGGGTGATGC |
| 132 | Ile_TAT_chr6: 285053 | GCTCCAGTGGCGCAATCGGTTAGCGCGCGGT |
| | 67-28505460 (+) | ACTTATAAGACAGTGCACCTGTGAGCAATGC |
| 133 | Leu_AAG_chr5: 1805 | GGTAGCGTGGCCGAGCGGTCTAAGGCGCTGG |
| | 24474-180524555 (−) | ATTAAGGCTCCAGTCTCTTCGGAGGCGTGGG |
| 134 | Leu_AAG_chr5: 1806 | GGTAGCGTGGCCGAGCGGTCTAAGGCGCTGG |
| | 14701-180614782 (+) | ATTAAGGCTCCAGTCTCTTCGGGGGCGTGGG |
| 135 | Leu_AAG_chr6: 2895 | GGTAGCGTGGCCGAGCGGTCTAAGGCGCTGG |
| | 6779-28956860 (+) | ATTAAGGCTCCAGTCTCTTCGGGGGCGTGGG |
| 136 | Leu_AAG_chr6: 2844 | GGTAGCGTGGCCGAGTGGTCTAAGACGCTGG |
| | 6400-28446481 (−) | ATTAAGGCTCCAGTCTCTTCGGGGGCGTGGG |
| 137 | Leu_CAA_chr6: 28864 | GTCAGGATGGCCGAGTGGTCTAAGGCGCCAG |
| | 000-28864105 (−) | ACTCAAGCTAAGCTTCCTCCGCGGTGGGGAT |
| 138 | Leu_CAA_chr6: 28908 | GTCAGGATGGCCGAGTGGTCTAAGGCGCCAG |
| | 830-28908934 (+) | ACTCAAGCTTGGCTTCCTCGTGTTGAGGATTC |
| 139 | Leu_CAA_chr6: 27573 | GTCAGGATGGCCGAGTGGTCTAAGGCGCCAG |
| | 417-27573524 (−) | ACTCAAGCTTACTGCTTCCTGTGTTCGGGTCT |
| 140 | Leu_CAA_chr6: 27570 | GTCAGGATGGCCGAGTGGTCTAAGGCGCCAG |
| | 348-27570454 (−) | ACTCAAGTTGCTACTTCCCAGGTTTGGGGCTT |
| 141 | Leu_CAA_chr1: 24916 | GTCAGGATGGCCGAGTGGTCTAAGGCGCCAG |
| | 8054-249168159 (+) | ACTCAAGGTAAGCACCTTGCCTGCGGGCTTT |
| 142 | Leu_CAA_chr11: 9296 | GCCTCCTTAGTGCAGTAGGTAGCGCATCAGT |
| | 790-9296863 (+) | CTCAAAATCTGAATGGTCCTGAGTTCAAGCC |
| 143 | Leu_CAA_chr1: 16158 | GTCAGGATGGCCGAGCAGTCTTAAGGCGCTG |
| | 1736-161581819 (−) | CGTTCAAATCGCACCCTCCGCTGGAGGCGTG |
| 144 | Leu_CAG_chr1: 16141 | GTCAGGATGGCCGAGCGGTCTAAGGCGCTGC |
| | 1323-161411405 (+) | GTTCAGGTCGCAGTCTCCCCTGGAGGCGTGG |
| 145 | Leu_CAG_chr16: 5733 | GTCAGGATGGCCGAGCGGTCTAAGGCGCTGC |
| | 3863-57333945 (+) | GTTCAGGTCGCAGTCTCCCCTGGAGGCGTGG |
| 146 | Leu_TAA_chr6: 14453 | ACCAGGATGGCCGAGTGGTTAAGGCGTTGGA |
| | 7684-144537766 (+) | CTTAAGATCCAATGGACATATGTCCGCGTGG |
| 147 | Leu_TAA_chr6: 27688 | ACCGGGATGGCCGAGTGGTTAAGGCGTTGGA |
| | 898-27688980 (−) | CTTAAGATCCAATGGGCTGGTGCCCGCGTGG |
| 148 | Leu_TAA_chr11: 5931 | ACCAGAATGGCCGAGTGGTTAAGGCGTTGGA |
| | 9228-59319310 (+) | CTTAAGATCCAATGGATTCATATCCGCGTGG |
| 149 | Leu_TAA_chr6: 27198 | ACCGGGATGGCTGAGTGGTTAAGGCGTTGGA |
| | 334-27198416 (−) | CTTAAGATCCAATGGACAGGTGTCCGCGTGG |
| 150 | Leu_TAG_chr17: 8023 | GGTAGCGTGGCCGAGCGGTCTAAGGCGCTGG |
| | 632-8023713 (−) | ATTTAGGCTCCAGTCTCTTCGGAGGCGTGGG |
| 151 | Leu_TAG_chr14: 2109 | GGTAGTGTGGCCGAGCGGTCTAAGGCGCTGG |
| | 3529-21093610 (+) | ATTTAGGCTCCAGTCTCTTCGGGGGCGTGGG |
| 152 | Leu_TAG_chr16: 2220 | GGTAGCGTGGCCGAGTGGTCTAAGGCGCTGG |
| | 7032-22207113 (−) | ATTTAGGCTCCAGTCATTTCGATGGCGTGGGT |
| 153 | Lys_CTT_chr14: 5870 | GCCCGGCTAGCTCAGTCGGTAGAGCATGGGA |
| | 6613-58706685 (−) | CTCTTAATCCCAGGGTCGTGGGTTCGAGCCC |
| 154 | Lys_CTT_chr19: 3606 | GCCCAGCTAGCTCAGTCGGTAGAGCATAAGA |
| | 6750-36066822 (+) | CTCTTAATCTCAGGGTTGTGGATTCGTGCCCC |
| 155 | Lys_CTT_chr19: 5242 | GCAGCTAGCTCAGTCGGTAGAGCATGAGACT |
| | 5393-52425466 (−) | CTTAATCTCAGGGTCATGGGTTCGTGCCCCAT |
| 156 | Lys_CTT_chr1: 14539 | GCCCGGCTAGCTCAGTCGGTAGAGCATGAGA |
| | 5522-145395594 (−) | CTCTTAATCTCAGGGTCGTGGGTTCGAGCCCC |
| 157 | Lys_CTT_chr16: 3207 | GCCCGGCTAGCTCAGTCGGTAGAGCATGAGA |
| | 406-3207478 (−) | CCCTTAATCTCAGGGTCGTGGGTTCGAGCCC |
| 158 | Lys_CTT_chr16: 3241 | GCCCGGCTAGCTCAGTCGGTAGAGCATGGGA |
| | 501-3241573 (+) | CTCTTAATCTCAGGGTCGTGGGTTCGAGCCCC |
| 159 | Lys_CTT_chr16: 3230 | GCCCGGCTAGCTCAGTCGATAGAGCATGAGA |
| | 555-3230627 (−) | CTCTTAATCTCAGGGTCGTGGGTTCGAGCCG |
| 160 | Lys_CTT_chr1: 55423 | GCCCAGCTAGCTCAGTCGGTAGAGCATGAGA |
| | 542-55423614 (−) | CTCTTAATCTCAGGGTCATGGGTTTGAGCCCC |
| 161 | Lys_CTT_chr16: 3214 | GCCTGGCTAGCTCAGTCGGCAAAGCATGAGA |
| | 939-3215011 (+) | CTCTTAATCTCAGGGTCGTGGGCTCGAGCTCC |
| 162 | Lys_CTT_chr5: 26198 | GCCCGACTACCTCAGTCGGTGGAGCATGGGA |
| | 539-26198611 (−) | CTCTTCATCCCAGGGTTGTGGGTTCGAGCCCC |
| 163 | Lys_TTT_chr16: 7351 | GCCTGGATAGCTCAGTTGGTAGAGCATCAGA |
| | 2216-73512288 (−) | CTTTTAATCTGAGGGTCCAGGGTTCAAGTCCC |
| 164 | Lys_TTT_chr12: 2784 | ACCCAGATAGCTCAGTCAGTAGAGCATCAGA |
| | 3306-27843378 (+) | CTTTTAATCTGAGGGTCCAAGGTTCATGTCCC |
| 165 | Lys_TTT_chr11: 1224 | GCCTGGATAGCTCAGTTGGTAGAGCATCAGA |
| | 30655-122430727 (+) | CTTTTAATCTGAGGGTCCAGGGTTCAAGTCCC |
| 166 | Lys_TTT_chr1: 20447 | GCCCGGATAGCTCAGTCGGTAGAGCATCAGA |
| | 5655-204475727 (+) | CTTTTAATCTGAGGGTCCAGGGTTCAAGTCCC |
| 167 | Lys_TTT_chr6: 27559 | GCCTGGATAGCTCAGTCGGTAGAGCATCAGA |
| | 593-27559665 (−) | CTTTTAATCTGAGGGTCCAGGGTTCAAGTCCC |
| 168 | Lys_TTT_chr11: 5932 | GCCCGGATAGCTCAGTCGGTAGAGCATCAGA |
| | 3902-59323974 (+) | CTTTTAATCTGAGGGTCCGGGGTTCAAGTCCC |
| 169 | Lys_TTT_chr6: 27302 | GCCTGGGTAGCTCAGTCGGTAGAGCATCAGA |
| | 769-27302841 (−) | CTTTTAATCTGAGGGTCCAGGGTTCAAGTCCC |
| 170 | Lys_TTT_chr6: 28715 | GCCTGGATAGCTCAGTTGGTAGAACATCAGA |
| | 521-28715593 (+) | CTTTTAATCTGACGGTGCAGGGTTCAAGTCCC |
| 171 | Met_CAT_chr8: 12416 | GCCTCGTTAGCGCAGTAGGTAGCGCGTCAGT |
| | 9470-124169542 (−) | CTCATAATCTGAAGGTCGTGAGTTCGATCCTC |
| 172 | Met_CAT_chr16: 7146 | GCCCTCTTAGCGCAGTGGGCAGCGCGTCAGT |
| | 0396-71460468 (+) | CTCATAATCTGAAGGTCCTGAGTTCGAGCCT |
| 173 | Met_CAT_chr6: 28912 | GCCTCCTTAGCGCAGTAGGCAGCGCGTCAGT |
| | 352-28912424 (+) | CTCATAATCTGAAGGTCCTGAGTTCGAACCT |
| 174 | Met_CAT_chr6: 26735 | GCCCTCTTAGCGCAGCGGGCAGCGCGTCAGT |
| | 574-26735646 (−) | CTCATAATCTGAAGGTCCTGAGTTCGAGCCT |
| 175 | Met_CAT_chr6: 26701 | GCCCTCTTAGCGCAGCTGGCAGCGCGTCAGT |
| | 712-26701784 (+) | CTCATAATCTGAAGGTCCTGAGTTCAAGCCT |
| 176 | Met_CAT_chr16: 8741 | GCCTCGTTAGCGCAGTAGGCAGCGCGTCAGT |
| | 7628-87417700 (−) | CTCATAATCTGAAGGTCGTGAGTTCGAGCCT |
| 177 | Met_CAT_chr6: 58168 | GCCCTCTTAGTGCAGCTGGCAGCGCGTCAGT |
| | 492-58168564 (−) | TTCATAATCTGAAAGTCCTGAGTTCAAGCCTC |
| 178 | Phe_GAA_chr6: 28758 | GCCGAAATAGCTCAGTTGGGAGAGCGTTAGA |
| | 499-28758571 (−) | CTGAAGATCTAAAGGTCCCTGGTTCGATCCC |
| 179 | Phe_GAA_chr11: 5933 | GCCGAAATAGCTCAGTTGGGAGAGCGTTAGA |
| | 3853-59333925 (−) | CTGAAGATCTAAAGGTCCCTGGTTCAATCCC |
| 180 | Phe_GAA_chr6: 28775 | GCCGAGATAGCTCAGTTGGGAGAGCGTTAGA |
| | 610-28775682 (−) | CTGAAGATCTAAAGGTCCCTGGTTCAATCCC |
| 181 | Phe_GAA_chr6: 28791 | GCCGAAATAGCTCAGTTGGGAGAGCGTTAGA |
| | 093-28791166 (−) | CCGAAGATCTTAAAGGTCCCTGGTTCAATCC |
| 182 | Phe_GAA_chr6: 28731 | GCTGAAATAGCTCAGTTGGGAGAGCGTTAGA |
| | 374-28731447 (−) | CTGAAGATCTTAAAGTTCCCTGGTTCAACCCT |
| 183 | Pro_AGG_chr16: 3241 | GGCTCGTTGGTCTAGGGGTATGATTCTCGCTT |
| | 989-3242060 (+) | AGGATGCGAGAGGTCCCGGGTTCAAATCCCG |
| 184 | Pro_AGG_chr1: 16768 | GGCTCGTTGGTCTAGGGGTATGATTCTCGCTT |
| | 4725-167684796 (−) | AGGGTGCGAGAGGTCCCGGGTTCAAATCCCG |
| 185 | Pro_CGG_chr1: 16768 | GGCTCGTTGGTCTAGGGGTATGATTCTCGCTT |
| | 3962-167684033 (+) | CGGGTGCGAGAGGTCCCGGGTTCAAATCCCG |
| 186 | Pro_CGG_chr6: 27059 | GGCTCGTTGGTCTAGGGGTATGATTCTCGCTT |
| | 521-27059592 (+) | CGGGTGTGAGAGGTCCCGGGTTCAAATCCCG |
| 187 | Pro_TGG_chr14: 2110 | GGCTCGTTGGTCTAGTGGTATGATTCTCGCTT |
| | 1165-21101236 (+) | TGGGTGCGAGAGGTCCCGGGTTCAAATCCCG |
| 188 | Pro_TGG_chr11: 7594 | GGCTCGTTGGTCTAGGGGTATGATTCTCGGTT |
| | 6869-75946940 (−) | TGGGTCCGAGAGGTCCCGGGTTCAAATCCCG |
| 189 | Pro_TGG_chr5: 18061 | GGCTCGTTGGTCTAGGGGTATGATTCTCGCTT |
| | 5854-180615925 (−) | TGGGTGCGAGAGGTCCCGGGTTCAAATCCCG |
| 190 | SeC_TCA_chr19: 4598 | GCCCGGATGATCCTCAGTGGTCTGGGGTGCA |
| | 1859-45981945 (−) | GGCTTCAAACCTGTAGCTGTCTAGCGACAGA |
| 191 | SeC_TCA_chr22: 4454 | GCTCGGATGATCCTCAGTGGTCTGGGGTGCA |
| | 6537-44546620 (+) | GGCTTCAAACCTGTAGCTGTCTAGTGACAGA |
| 192 | Ser_AGA_chr6: 27509 | GTAGTCGTGGCCGAGTGGTTAAGGCGATGGA |
| | 554-27509635 (−) | CTAGAAATCCATTGGGGTTTCCCCGCGCAGG |
| 193 | Ser_AGA_chr6: 26327 | GTAGTCGTGGCCGAGTGGTTAAGGCGATGGA |
| | 817-26327898 (+) | CTAGAAATCCATTGGGGTCTCCCCGCGCAGG |
| 194 | Ser_AGA_chr6: 27499 | GTAGTCGTGGCCGAGTGGTTAAGGCGATGGA |
| | 987-27500068 (+) | CTAGAAATCCATTGGGGTTTCCCCACGCAGG |
| 195 | Ser_AGA_chr6: 27521 | GTAGTCGTGGCCGAGTGGTTAAGGTGATGGA |
| | 192-27521273 (−) | CTAGAAACCCATTGGGGTCTCCCCGCGCAGG |
| 196 | Ser_CGA_chr17: 8042 | GCTGTGATGGCCGAGTGGTTAAGGCGTTGGA |
| | 199-8042280 (−) | CTCGAAATCCAATGGGGTCTCCCCGCGCAGG |
| 197 | Ser_CGA_chr6: 27177 | GCTGTGATGGCCGAGTGGTTAAGGCGTTGGA |
| | 628-27177709 (+) | CTCGAAATCCAATGGGGTCTCCCCGCGCAGG |
| 198 | Ser_CGA_chr6: 27640 | GCTGTGATGGCCGAGTGGTTAAGGTGTTGGA |
| | 229-27640310 (−) | CTCGAAATCCAATGGGGGTTCCCCGCGCAGG |
| 199 | Ser_CGA_chr12: 5658 | GTCACGGTGGCCGAGTGGTTAAGGCGTTGGA |
| | 4148-56584229 (+) | CTCGAAATCCAATGGGGTTTCCCCGCACAGG |
| 200 | Ser_GCT_chr6: 27065 | GACGAGGTGGCCGAGTGGTTAAGGCGATGG |
| | 085-27065166 (+) | ACTGCTAATCCATTGTGCTCTGCACGCGTGG |
| 201 | Ser_GCT_chr6: 27265 | GACGAGGTGGCCGAGTGGTTAAGGCGATGG |
| | 775-27265856 (+) | ACTGCTAATCCATTGTGCTCTGCACGCGTGG |
| 202 | Ser_GCT_chr11: 6611 | GACGAGGTGGCCGAGTGGTTAAGGCGATGG |
| | 5591-66115672 (+) | ACTGCTAATCCATTGTGCTTTGCACGCGTGGG |
| 203 | Ser_GCT_chr6: 28565 | GACGAGGTGGCCGAGTGGTTAAGGCGATGG |
| | 117-28565198 (−) | ACTGCTAATCCATTGTGCTCTGCACGCGTGG |
| 204 | Ser_GCT_chr6: 28180 | GACGAGGTGGCCGAGTGGTTAAGGCGATGG |
| | 815-28180896 (+) | ACTGCTAATCCATTGTGCTCTGCACACGTGG |
| 205 | Ser_GCT_chr6: 26305 | GGAGAGGCCTGGCCGAGTGGTTAAGGCGATG |
| | 718-26305801 (−) | GACTGCTAATCCATTGTGCTCTGCACGCGTG |
| 206 | Ser_TGA_chr10: 6952 | GCAGCGATGGCCGAGTGGTTAAGGCGTTGGA |
| | 4261-69524342 (+) | CTTGAAATCCAATGGGGTCTCCCCGCGCAGG |
| 207 | Ser_TGA_chr6: 27513 | GTAGTCGTGGCCGAGTGGTTAAGGCGATGGA |
| | 468-27513549 (+) | CTTGAAATCCATTGGGGTTTCCCCGCGCAGG |
| 208 | Ser_TGA_chr6: 26312 | GTAGTCGTGGCCGAGTGGTTAAGGCGATGGA |
| | 824-26312905 (−) | CTTGAAATCCATTGGGGTCTCCCCGCGCAGG |
| 209 | Ser_TGA_chr6: 27473 | GTAGTCGTGGCCGAGTGGTTAAGGCGATGGA |
| | 607-27473688 (−) | CTTGAAATCCATTGGGGTTTCCCCGCGCAGG |
| 210 | Thr_AGT_chr17: 8090 | GGCGCCGTGGCTTAGTTGGTTAAAGCGCCTG |
| | 478-8090551 (+) | TCTAGTAAACAGGAGATCCTGGGTTCGAATC |
| 211 | Thr_AGT_chr6: 26533 | GGCTCCGTGGCTTAGCTGGTTAAAGCGCCTG |
| | 145-26533218 (−) | TCTAGTAAACAGGAGATCCTGGGTTCGAATC |
| 212 | Thr_AGT_chr6: 28693 | GGCTCCGTAGCTTAGTTGGTTAAAGCGCCTG |
| | 795-28693868 (+) | TCTAGTAAACAGGAGATCCTGGGTTCGACTC |
| 213 | Thr_AGT_chr6: 27694 | GGCTTCGTGGCTTAGCTGGTTAAAGCGCCTG |
| | 473-27694546 (+) | TCTAGTAAACAGGAGATCCTGGGTTCGAATC |
| 214 | Thr_AGT_chr17: 8042 | GGCGCCGTGGCTTAGCTGGTTAAAGCGCCTG |
| | 770-8042843 (−) | TCTAGTAAACAGGAGATCCTGGGTTCGAATC |
| 215 | Thr_AGT_chr6: 27130 | GGCCCTGTGGCTTAGCTGGTCAAAGCGCCTG |
| | 050-27130123 (+) | TCTAGTAAACAGGAGATCCTGGGTTCGAATC |
| 216 | Thr_CGT_chr6: 28456 | GGCTCTATGGCTTAGTTGGTTAAAGCGCCTGT |
| | 770-28456843 (−) | CTCGTAAACAGGAGATCCTGGGTTCGACTCC |
| 217 | Thr_CGT_chr16: 1437 | GGCGCGGTGGCCAAGTGGTAAGGCGTCGGTC |
| | 9750-14379821 (+) | TCGTAAACCGAAGATCACGGGTTCGAACCCC |
| 218 | Thr_CGT_chr6: 28615 | GGCTCTGTGGCTTAGTTGGCTAAAGCGCCTG |
| | 984-28616057 (−) | TCTCGTAAACAGGAGATCCTGGGTTCGAATC |
| 219 | Thr_CGT_chr17: 2987 | GGCGCGGTGGCCAAGTGGTAAGGCGTCGGTC |
| | 7093-29877164 (+) | TCGTAAACCGAAGATCGCGGGTTCGAACCCC |
| 220 | Thr_CGT_chr6: 27586 | GGCCCTGTAGCTCAGCGGTTGGAGCGCTGGT |
| | 135-27586208 (+) | CTCGTAAACCTAGGGGTCGTGAGTTCAAATC |
| 221 | Thr_TGT_chr6: 28442 | GGCTCTATGGCTTAGTTGGTTAAAGCGCCTGT |
| | 329-28442402 (−) | CTTGTAAACAGGAGATCCTGGGTTCGAATCC |
| 222 | Thr_TGT_chr1: 22263 | GGCTCCATAGCTCAGTGGTTAGAGCACTGGT |
| | 8347-222638419 (+) | CTTGTAAACCAGGGGTCGCGAGTTCGATCCT |
| 223 | Thr_TGT_chr14: 2108 | GGCTCCATAGCTCAGGGGTTAGAGCGCTGGT |
| | 1949-21082021 (−) | CTTGTAAACCAGGGGTCGCGAGTTCAATTCT |
| 224 | Thr_TGT_chr14: 2109 | GGCTCCATAGCTCAGGGGTTAGAGCACTGGT |
| | 9319-21099391 (−) | CTTGTAAACCAGGGGTCGCGAGTTCAAATCT |
| 225 | Thr_TGT_chr14: 2114 | GGCCCTATAGCTCAGGGGTTAGAGCACTGGT |
| | 9849-21149921 (+) | CTTGTAAACCAGGGGTCGCGAGTTCAAATCT |
| 226 | Thr_TGT_chr5: 18061 | GGCTCCATAGCTCAGGGGTTAGAGCACTGGT |
| | 8687-180618758 (−) | CTTGTAAACCAGGGTCGCGAGTTCAAATCTC |
| 227 | Trp_CCA_chr17: 8124 | GGCCTCGTGGCGCAACGGTAGCGCGTCTGAC |
| | 187-8124258 (−) | TCCAGATCAGAAGGTTGCGTGTTCAAATCAC |
| 228 | Trp_CCA_chr17: 1941 | GACCTCGTGGCGCAATGGTAGCGCGTCTGAC |
| | 1494-19411565 (+) | TCCAGATCAGAAGGTTGCGTGTTCAAGTCAC |
| 229 | Trp_CCA_chr6: 26319 | GACCTCGTGGCGCAACGGTAGCGCGTCTGAC |
| | 330-26319401 (−) | TCCAGATCAGAAGGTTGCGTGTTCAAATCAC |
| 230 | Trp_CCA_chr12: 9889 | GACCTCGTGGCGCAACGGTAGCGCGTCTGAC |
| | 8030-98898101 (+) | TCCAGATCAGAAGGCTGCGTGTTCGAATCAC |
| 231 | Trp_CCA_chr7: 99067 | GACCTCGTGGCGCAACGGCAGCGCGTCTGAC |
| | 307-99067378 (+) | TCCAGATCAGAAGGTTGCGTGTTCAAATCAC |
| 232 | Tyr_ATA_chr2: 21911 | CCTTCAATAGTTCAGCTGGTAGAGCAGAGGA |
| | 0549-219110641 (+) | CTATAGCTACTTCCTCAGTAGGAGACGTCCTT |
| 233 | Tyr_GTA_chr6: 26569 | CCTTCGATAGCTCAGTTGGTAGAGCGGAGGA |
| | 086-26569176 (+) | CTGTAGTTGGCTGTGTCCTTAGACATCCTTAG |
| 234 | Tyr_GTA_chr2: 27273 | CCTTCGATAGCTCAGTTGGTAGAGCGGAGGA |
| | 650-27273738 (+) | CTGTAGTGGATAGGGCGTGGCAATCCTTAGG |
| 235 | Tyr_GTA_chr6: 26577 | CCTTCGATAGCTCAGTTGGTAGAGCGGAGGA |
| | 332-26577420 (+) | CTGTAGGCTCATTAAGCAAGGTATCCTTAGG |
| 236 | Tyr_GTA_chr14: 2112 | CCTTCGATAGCTCAGCTGGTAGAGCGGAGGA |
| | 5623-21125716 (−) | CTGTAGATTGTATAGACATTTGCGGACATCCT |
| 237 | Tyr_GTA_chr8: 67025 | CCTTCGATAGCTCAGCTGGTAGAGCGGAGGA |
| | 602-67025694 (+) | CTGTAGCTACTTCCTCAGCAGGAGACATCCTT |
| 238 | Tyr_GTA_chr8: 67026 | CCTTCGATAGCTCAGCTGGTAGAGCGGAGGA |
| | 223-67026311 (+) | CTGTAGGCGCGCGCCCGTGGCCATCCTTAGG |
| 239 | Tyr_GTA_chr14: 2112 | CCTTCGATAGCTCAGCTGGTAGAGCGGAGGA |
| | 1258-21121351 (−) | CTGTAGCCTGTAGAAACATTTGTGGACATCC |
| 240 | Tyr_GTA_chr14: 2113 | CCTTCGATAGCTCAGCTGGTAGAGCGGAGGA |
| | 1351-21131444 (−) | CTGTAGATTGTACAGACATTTGCGGACATCC |
| 241 | Tyr_GTA_chr14: 2115 | CCTTCGATAGCTCAGCTGGTAGAGCGGAGGA |
| | 1432-21151520 (+) | CTGTAGTACTTAATGTGTGGTCATCCTTAGGT |
| 242 | Tyr_GTA_chr6: 26595 | CCTTCGATAGCTCAGCTGGTAGAGCGGAGGA |
| | 102-26595190 (+) | CTGTAGGGGTTTGAATGTGGTCATCCTTAGGT |
| 243 | Tyr_GTA_chr14: 2112 | CCTTCGATAGCTCAGCTGGTAGAGCGGAGGA |
| | 8117-21128210 (−) | CTGTAGACTGCGGAAACGTTTGTGGACATCC |
| 244 | Tyr_GTA_chr6: 26575 | CTTTCGATAGCTCAGTTGGTAGAGCGGAGGA |
| | 798-26575887 (+) | CTGTAGGTTCATTAAACTAAGGCATCCTTAG |
| 245 | Tyr_GTA_chr8: 66609 | TCTTCAATAGCTCAGCTGGTAGAGCGGAGGA |
| | 532-66609619 (−) | CTGTAGGTGCACGCCCGTGGCCATTCTTAGG |
| 246 | Val_AAC_chr3: 16949 | GTTTCCGTAGTGTAGTGGTTATCACGTTCGCC |
| | 0018-169490090 (+) | TAACACGCGAAAGGTCCCCGGTTCGAAACCG |
| 247 | Val_AAC_chr5: 18061 | GTTTCCGTAGTGTAGTGGTCATCACGTTCGCC |
| | 5416-180615488 (−) | TAACACGCGAAAGGTCCCCGGTTCGAAACCG |
| 248 | Val_AAC_chr6: 27618 | GTTTCCGTAGTGTAGTGGTTATCACGTTCGCC |
| | 707-27618779 (−) | TAACACGCGAAAGGTCCCTGGATCAAAACCA |
| 249 | Val_AAC_chr6: 27648 | GTTTCCGTAGTGTAGTGGTTATCACGTTCGCC |
| | 885-27648957 (−) | TAACACGCGAAAGGTCCGCGGTTCGAAACCG |
| 250 | Val_AAC_chr6: 27203 | GTTTCCGTAGTGTAGTGGTTATCACGTTTGCC |
| | 288-27203360 (+) | TAACACGCGAAAGGTCCCCGGTTCGAAACCG |
| 251 | Val_AAC_chr6: 28703 | GGGGGTGTAGCTCAGTGGTAGAGCGTATGCT |
| | 206-28703277 (−) | TAACATTCATGAGGCTCTGGGTTCGATCCCC |
| 252 | Val_CAC_chr1: 16136 | GTTTCCGTAGTGTAGTGGTTATCACGTTCGCC |
| | 9490-161369562 (−) | TCACACGCGAAAGGTCCCCGGTTCGAAACCG |
| 253 | Val_CAC_chr6: 27248 | GCTTCTGTAGTGTAGTGGTTATCACGTTCGCC |
| | 049-27248121 (−) | TCACACGCGAAAGGTCCCCGGTTCGAAACCG |
| 254 | Val_CAC_chr19: 4724 | GTTTCCGTAGTGTAGCGGTTATCACATTCGCC |
| | 647-4724719 (−) | TCACACGCGAAAGGTCCCCGGTTCGATCCCG |
| 255 | Val_CAC_chr1: 14929 | GTTTCCGTAGTGTAGTGGTTATCACGTTCGCC |
| | 8555-149298627 (−) | TCACACGCGAAAGGTCCCCGGTTCGAAACTG |
| 256 | Val_CAC_chr1: 14968 | GTTTCCGTAGTGTAGTGGTTATCACGTTCGCC |
| | 4088-149684161 (−) | TCACACGCGTAAAGGTCCCCGGTTCGAAACC |
| 257 | Val_CAC_chr6: 27173 | GTTTCCGTAGTGGAGTGGTTATCACGTTCGCC |
| | 867-27173939 (−) | TCACACGCGAAAGGTCCCCGGTTTGAAACCA |
| 258 | Val_TAC_chr11: 5931 | GGTTCCATAGTGTAGTGGTTATCACGTCTGCT |
| | 8102-59318174 (−) | TTACACGCAGAAGGTCCTGGGTTCGAGCCCC |
| 259 | Val_TAC_chr11: 5931 | GGTTCCATAGTGTAGCGGTTATCACGTCTGCT |
| | 8460-59318532 (−) | TTACACGCAGAAGGTCCTGGGTTCGAGCCCC |
| 260 | Val_TAC_chr10: 5895 | GGTTCCATAGTGTAGTGGTTATCACATCTGCT |
| | 674-5895746 (−) | TTACACGCAGAAGGTCCTGGGTTCAAGCCCC |
| 261 | Val_TAC_chr6: 27258 | GTTTCCGTGGTGTAGTGGTTATCACATTCGCC |
| | 405-27258477 (+) | TTACACGCGAAAGGTCCTCGGGTCGAAACCG |
| 262 | iMet_CAT_chr1: 1536 | AGCAGAGTGGCGCAGCGGAAGCGTGCTGGG |
| | 43726-153643797 (+) | CCCATAACCCAGAGGTCGATGGATCGAAACC |
| 263 | iMet_CAT_chr6: 2774 | AGCAGAGTGGCGCAGCGGAAGCGTGCTGGG |
| | 5664-27745735 (+) | CCCATAACCCAGAGGTCGATGGATCTAAACC |
| 264 | Glu_TTC_chr1: 16861 | TCCCTGGTGGTCTAGTGGCTAGGATTCGGCG |
| | 773-16861845 (−) | CTTTCACCGCCGCGGCCCGGGTTCGATTCCCG |
| 265 | Gly_CCC_chr1: 17004 | GCGTTGGTGGTTTAGTGGTAGAATTCTCGCCT |
| | 765-17004836 (−) | CCCATGCGGGAGACCCGGGTTCAATTCCCGG |
| 266 | Gly_CCC_chr1: 17053 | GGCCTTGGTGGTGCAGTGGTAGAATTCTCGC |
| | 779-17053850 (+) | CTCCCACGTGGGAGACCCGGGTTCAATTCCC |
| 267 | Glu_TTC_chr1: 17199 | GTCCCTGGTGGTCTAGTGGCTAGGATTCGGC |
| | 077-17199149 (+) | GCTTTCACCGCCGCGGCCCGGGTTCGATTCCC |
| 268 | Asn_GTT_chr1: 17216 | TGTCTCTGTGGCGCAATCGGTTAGCGCGTTCG |
| | 171-17216245 (+) | GCTGTTAACCGAAAGATTGGTGGTTCGAGCC |
| 269 | Arg_TCT_chr1: 94313 | TGGCTCCGTGGCGCAATGGATAGCGCATTGG |
| | 128-94313213 (+) | ACTTCTAGAGGCTGAAGGCATTCAAAGGTTC |
| 270 | Lys_CTT_chr1: 14539 | GCCCGGCTAGCTCAGTCGGTAGAGCATGAGA |
| | 5521-145395594 (−) | CTCTTAATCTCAGGGTCGTGGGTTCGAGCCCC |
| 271 | His_GTG_chr1: 14539 | GCCGTGATCGTATAGTGGTTAGTACTCTGCGT |
| | 6880-145396952 (−) | TGTGGCCGCAGCAACCTCGGTTCGAATCCGA |
| 272 | Gly_TCC_chr1: 14539 | GCGTTGGTGGTATAGTGGTGAGCATAGCTGC |
| | 7863-145397935 (−) | CTTCCAAGCAGTTGACCCGGGTTCGATTCCC |
| 273 | Glu_CTC_chr1: 14539 | TCCCTGGTGGTCTAGTGGTTAGGATTCGGCG |
| | 9232-145399304 (−) | CTCTCACCGCCGCGGCCCGGGTTCGATTCCC |
| 274 | Gln_CTG_chr1: 14596 | AGGTTCCATGGTGTAATGGTGAGCACTCTGG |
| | 3303-145963375 (+) | ACTCTGAATCCAGCGATCCGAGTTCGAGTCT |
| 275 | Asn_GTT_chr1: 14800 | TGTCTCTGTGGCGTAGTCGGTTAGCGCGTTCG |
| | 0804-148000878 (+) | GCTGTTAACCGAAAAGTTGGTGGTTCGAGCC |
| 276 | Asn_GTT_chr1: 14824 | TGTCTCTGTGGCGCAATCGGTTAGCGCGTTCG |
| | 8114-148248188 (+) | GCTGTTAACCGAAAGGTTGGTGGTTCGAGCC |
| 277 | Asn_GTT_chr1: 14859 | GTCTCTGTGGCGCAATCGGTTAGCGCATTCG |
| | 8313-148598387 (−) | GCTGTTAACCGAAAGGTTGGTGGTTCGAGCC |
| 278 | Asn_GTT_chr1: 14923 | GTCTCTGTGGCGCAATGGGTTAGCGCGTTCG |
| | 0569-149230643 (−) | GCTGTTAACCGAAAGGTTGGTGGTTCGAGCC |
| 279 | Val_CAC_chr1: 14929 | GCACTGGTGGTTCAGTGGTAGAATTCTCGCC |
| | 4665-149294736 (−) | TCACACGCGGGACACCCGGGTTCAATTCCCG |
| 280 | Val_CAC_chr1: 14929 | GTTTCCGTAGTGTAGTGGTTATCACGTTCGCC |
| | 8554-149298627 (−) | TCACACGCGAAAGGTCCCCGGTTCGAAACTG |
| 281 | Gly_CCC_chr1: 14968 | GCACTGGTGGTTCAGTGGTAGAATTCTCGCC |
| | 0209-149680280 (−) | TCCCACGCGGGAGACCCGGGTTTAATTCCCG |
| 282 | Val_CAC_chr1: 14968 | GTTTCCGTAGTGTAGTGGTTATCACGTTCGCC |
| | 4087-149684161 (−) | TCACACGCGTAAAGGTCCCCGGTTCGAAACC |
| 283 | Met_CAT_chr1: 15364 | TAGCAGAGTGGCGCAGCGGAAGCGTGCTGG |
| | 3725-153643797 (+) | GCCCATAACCCAGAGGTCGATGGATCGAAAC |
| 284 | Val_CAC_chr1: 16136 | GTTTCCGTAGTGTAGTGGTTATCACGTTCGCC |
| | 9489-161369562 (−) | TCACACGCGAAAGGTCCCCGGTTCGAAACCG |
| 285 | Asp_GTC_chr1: 16141 | TCCTCGTTAGTATAGTGGTGAGTATCCCCGCC |
| | 0614-161410686 (−) | TGTCACGCGGGAGACCGGGGTTCGATTCCCC |
| 286 | Gly_GCC_chr1: 16141 | TGCATGGGTGGTTCAGTGGTAGAATTCTCGC |
| | 3093-161413164 (+) | CTGCCACGCGGGAGGCCCGGGTTCGATTCCC |
| 287 | Glu_CTC_chr1: 16141 | TCCCTGGTGGTCTAGTGGTTAGGATTCGGCG |
| | 7017-161417089 (−) | CTCTCACCGCCGCGGCCCGGGTTCGATTCCC |
| 288 | Asp_GTC_chr1: 16149 | ATCCTTGTTACTATAGTGGTGAGTATCTCTGC |
| | 2934-161493006 (+) | CTGTCATGCGTGAGAGAGGGGGTCGATTCCC |
| 289 | Gly_GCC_chr1: 16149 | GCATTGGTGGTTCAGTGGTAGAATTCTCGCCT |
| | 3636-161493707 (−) | GCCACGCGGGAGGCCCGGGTTCGATTCCCGG |
| 290 | Leu_CAG_chr1: 16150 | GTCAGGATGGCCGAGCGGTCTAAGGCGCTGC |
| | 0131-161500214 (−) | GTTCAGGTCGCAGTCTCCCCTGGAGGCGTGG |
| 291 | Gly_TCC_chr1: 16150 | CGCGTTGGTGGTATAGTGGTGAGCATAGCTG |
| | 0902-161500974 (+) | CCTTCCAAGCAGTTGACCCGGGTTCGATTCCC |
| 292 | Asn_GTT_chr1: 16151 | CGTCTCTGTGGCGCAATCGGTTAGCGCGTTC |
| | 0030-161510104 (+) | GGCTGTTAACCGAAAGGTTGGTGGTTCGATC |
| 293 | Glu_TTC_chr1: 16158 | CGCGTTGGTGGTGTAGTGGTGAGCACAGCTG |
| | 2507-161582579 (+) | CCTTTCAAGCAGTTAACGCGGGTTCGATTCCC |
| 294 | Pro_CGG_chr1: 16768 | CGGCTCGTTGGTCTAGGGGTATGATTCTCGCT |
| | 3961-167684033 (+) | TCGGGTGCGAGAGGTCCCGGGTTCAAATCCC |
| 295 | Pro_AGG_chr1: 16768 | GGCTCGTTGGTCTAGGGGTATGATTCTCGCTT |
| | 4724-167684796 (−) | AGGGTGCGAGAGGTCCCGGGTTCAAATCCCG |
| 296 | Lys_TTT_chr1: 20447 | CGCCCGGATAGCTCAGTCGGTAGAGCATCAG |
| | 5654-204475727 (+) | ACTTTTAATCTGAGGGTCCAGGGTTCAAGTC |
| 297 | Lys_TTT_chr1: 20447 | GCCCGGATAGCTCAGTCGGTAGAGCATCAGA |
| | 6157-204476230 (−) | CTTTTAATCTGAGGGTCCAGGGTTCAAGTCCC |
| 298 | Leu_CAA_chr1: 24916 | TGTCAGGATGGCCGAGTGGTCTAAGGCGCCA |
| | 8053-249168159 (+) | GACTCAAGGTAAGCACCTTGCCTGCGGGCTT |
| 299 | Glu_CTC_chr1: 24916 | TTCCCTGGTGGTCTAGTGGTTAGGATTCGGCG |
| | 8446-249168518 (+) | CTCTCACCGCCGCGGCCCGGGTTCGATTCCC |
| 300 | Tyr_GTA_chr2: 27273 | GCCTTCGATAGCTCAGTTGGTAGAGCGGAGG |
| | 649-27273738 (+) | ACTGTAGTGGATAGGGCGTGGCAATCCTTAG |
| 301 | Ala_AGC_chr2: 27274 | CGGGGGATTAGCTCAAATGGTAGAGCGCTCG |
| | 081-27274154 (+) | CTTAGCATGCGAGAGGTAGCGGGATCGATGC |
| 302 | Ile_TAT_chr2: 430376 | AGCTCCAGTGGCGCAATCGGTTAGCGCGCGG |
| | 75-43037768 (+) | TACTTATACAGCAGTACATGCAGAGCAATGC |
| 303 | Gly_CCC_chr2: 70476 | GCGCCGCTGGTGTAGTGGTATCATGCAAGAT |
| | 122-70476193 (−) | TCCCATTCTTGCGACCCGGGTTCGATTCCCGG |
| 304 | Glu_TTC_chr2: 13109 | TCCCATATGGTCTAGCGGTTAGGATTCCTGGT |
| | 4700-131094772 (−) | TTTCACCCAGGTGGCCCGGGTTCGACTCCCG |
| 305 | Ala_CGC_chr2: 15725 | GGGGGATGTAGCTCAGTGGTAGAGCGCGCGC |
| | 7280-157257352 (+) | TTCGCATGTGTGAGGTCCCGGGTTCAATCCCC |
| 306 | Gly_GCC_chr2: 15725 | GCATTGGTGGTTCAGTGGTAGAATTCTCGCCT |
| | 7658-157257729 (−) | GCCACGCGGGAGGCCCGGGTTCGATTCCCGG |
| 307 | Arg_ACG_chr3: 45730 | GGGCCAGTGGCGCAATGGATAACGCGTCTGA |
| | 490-45730563 (−) | CTACGGATCAGAAGATTCTAGGTTCGACTCC |
| 308 | Val_AAC_chr3: 16949 | GGTTTCCGTAGTGTAGTGGTTATCACGTTCGC |
| | 0017-169490090 (+) | CTAACACGCGAAAGGTCCCCGGTTCGAAACC |
| 309 | Val_AAC_chr5: 18059 | AGTTTCCGTAGTGTAGTGGTTATCACGTTCGC |
| | 6609-180596682 (+) | CTAACACGCGAAAGGTCCCCGGTTCGAAACC |
| 310 | Leu_AAG_chr5: 1806 | AGGTAGCGTGGCCGAGCGGTCTAAGGCGCTG |
| | 14700-180614782 (+) | GATTAAGGCTCCAGTCTCTTCGGGGGCGTGG |
| 311 | Val_AAC_chr5: 18061 | GTTTCCGTAGTGTAGTGGTCATCACGTTCGCC |
| | 5415-180615488 (−) | TAACACGCGAAAGGTCCCCGGTTCGAAACCG |
| 312 | Pro_TGG_chr5: 18061 | GGCTCGTTGGTCTAGGGGTATGATTCTCGCTT |
| | 5853-180615925 (−) | TGGGTGCGAGAGGTCCCGGGTTCAAATCCCG |
| 313 | Thr_TGT_chr5: 18061 | GGCTCCATAGCTCAGGGGTTAGAGCACTGGT |
| | 8686-180618758 (−) | CTTGTAAACCAGGGTCGCGAGTTCAAATCTC |
| 314 | Ala_TGC_chr5: 18063 | TGGGGATGTAGCTCAGTGGTAGAGCGCATGC |
| | 3867-180633939 (+) | TTTGCATGTATGAGGCCCCGGGTTCGATCCCC |
| 315 | Lys_CTT_chr5: 18063 | CGCCCGGCTAGCTCAGTCGGTAGAGCATGAG |
| | 4754-180634827 (+) | ACTCTTAATCTCAGGGTCGTGGGTTCGAGCC |
| 316 | Val_AAC_chr5: 18064 | GTTTCCGTAGTGTAGTGGTTATCACGTTCGCC |
| | 5269-180645342 (−) | TAACACGCGAAAGGTCCCCGGTTCGAAACCG |
| 317 | Lys_CTT_chr5: 18064 | GCCCGGCTAGCTCAGTCGGTAGAGCATGAGA |
| | 8978-180649051 (−) | CTCTTAATCTCAGGGTCGTGGGTTCGAGCCCC |
| 318 | Val_CAC_chr5: 18064 | GTTTCCGTAGTGTAGTGGTTATCACGTTCGCC |
| | 9394-180649467 (−) | TCACACGCGAAAGGTCCCCGGTTCGAAACCG |
| 319 | Met_CAT_chr6: 26286 | CAGCAGAGTGGCGCAGCGGAAGCGTGCTGG |
| | 753-26286825 (+) | GCCCATAACCCAGAGGTCGATGGATCGAAAC |
| 320 | Ser_GCT_chr6: 26305 | GGAGAGGCCTGGCCGAGTGGTTAAGGCGATG |
| | 717-26305801 (−) | GACTGCTAATCCATTGTGCTCTGCACGCGTG |
| 321 | Gln_TTG_chr6: 26311 | GGCCCCATGGTGTAATGGTTAGCACTCTGGA |
| | 423-26311495 (−) | CTTTGAATCCAGCGATCCGAGTTCAAATCTC |
| 322 | Gln_TTG_chr6: 26311 | GGCCCCATGGTGTAATGGTTAGCACTCTGGA |
| | 974-26312046 (−) | CTTTGAATCCAGCGATCCGAGTTCAAATCTC |
| 323 | Ser_TGA_chr6: 26312 | GTAGTCGTGGCCGAGTGGTTAAGGCGATGGA |
| | 823-26312905 (−) | CTTGAAATCCATTGGGGTCTCCCCGCGCAGG |
| 324 | Met_CAT_chr6: 26313 | AGCAGAGTGGCGCAGCGGAAGCGTGCTGGG |
| | 351-26313423 (−) | CCCATAACCCAGAGGTCGATGGATCGAAACC |
| 325 | Arg_TCG_chr6: 26323 | GGACCACGTGGCCTAATGGATAAGGCGTCTG |
| | 045-26323118 (+) | ACTTCGGATCAGAAGATTGAGGGTTCGAATC |
| 326 | Ser_AGA_chr6: 26327 | TGTAGTCGTGGCCGAGTGGTTAAGGCGATGG |
| | 816-26327898 (+) | ACTAGAAATCCATTGGGGTCTCCCCGCGCAG |
| 327 | Met_CAT_chr6: 26330 | AGCAGAGTGGCGCAGCGGAAGCGTGCTGGG |
| | 528-26330600 (−) | CCCATAACCCAGAGGTCGATGGATCGAAACC |
| 328 | Leu_CAG_chr6: 26521 | CGTCAGGATGGCCGAGCGGTCTAAGGCGCTG |
| | 435-26521518 (+) | CGTTCAGGTCGCAGTCTCCCCTGGAGGCGTG |
| 329 | Thr_AGT_chr6: 26533 | GGCTCCGTGGCTTAGCTGGTTAAAGCGCCTG |
| | 144-26533218 (−) | TCTAGTAAACAGGAGATCCTGGGTTCGAATC |
| 330 | Arg_ACG_chr6: 26537 | AGGGCCAGTGGCGCAATGGATAACGCGTCTG |
| | 725-26537798 (+) | ACTACGGATCAGAAGATTCCAGGTTCGACTC |
| 331 | Val_CAC_chr6: 26538 | GGTTTCCGTAGTGTAGTGGTTATCACGTTCGC |
| | 281-26538354 (+) | CTCACACGCGAAAGGTCCCCGGTTCGAAACC |
| 332 | Ala_CGC_chr6: 26553 | AGGGGATGTAGCTCAGTGGTAGAGCGCATGC |
| | 730-26553802 (+) | TTCGCATGTATGAGGTCCCGGGTTCGATCCCC |
| 333 | Ile_AAT_chr6: 265543 | TGGCCGGTTAGCTCAGTTGGTTAGAGCGTGG |
| | 49-26554423 (+) | TGCTAATAACGCCAAGGTCGCGGGTTCGATC |
| 334 | Pro_AGG_chr6: 26555 | CGGCTCGTTGGTCTAGGGGTATGATTCTCGCT |
| | 497-26555569 (+) | TAGGGTGCGAGAGGTCCCGGGTTCAAATCCC |
| 335 | Lys_CTT_chr6: 26556 | AGCCCGGCTAGCTCAGTCGGTAGAGCATGAG |
| | 773-26556846 (+) | ACTCTTAATCTCAGGGTCGTGGGTTCGAGCC |
| 336 | Tyr_GTA_chr6: 26569 | TCCTTCGATAGCTCAGTTGGTAGAGCGGAGG |
| | 085-26569176 (+) | ACTGTAGTTGGCTGTGTCCTTAGACATCCTTA |
| 337 | Ala_AGC_chr6: 26572 | GGGGAATTAGCTCAAATGGTAGAGCGCTCGC |
| | 091-26572164 (−) | TTAGCATGCGAGAGGTAGCGGGATCGATGCC |
| 338 | Met_CAT_chr6: 26766 | CGCCCTCTTAGCGCAGCGGGCAGCGCGTCAG |
| | 443-26766516 (+) | TCTCATAATCTGAAGGTCCTGAGTTCGAGCCT |
| 339 | Ile_TAT_chr6: 269881 | TGCTCCAGTGGCGCAATCGGTTAGCGCGCGG |
| | 24-26988218 (+) | TACTTATATGGCAGTATGTGTGCGAGTGATG |
| 340 | His_GTG_chr6: 27125 | TGCCGTGATCGTATAGTGGTTAGTACTCTGCG |
| | 905-27125977 (+) | TTGTGGCCGCAGCAACCTCGGTTCGAATCCG |
| 341 | Ile_AAT_chr6: 271449 | GGCCGGTTAGCTCAGTTGGTTAGAGCGTGGT |
| | 93-27145067 (−) | GCTAATAACGCCAAGGTCGCGGGTTCGATCC |
| 342 | Val_AAC_chr6: 27203 | AGTTTCCGTAGTGTAGTGGTTATCACGTTTGC |
| | 287-27203360 (+) | CTAACACGCGAAAGGTCCCCGGTTCGAAACC |
| 343 | Val_CAC_chr6: 27248 | GCTTCTGTAGTGTAGTGGTTATCACGTTCGCC |
| | 048-27248121 (−) | TCACACGCGAAAGGTCCCCGGTTCGAAACCG |
| 344 | Asp_GTC_chr6: 27447 | TTCCTCGTTAGTATAGTGGTGAGTATCCCCGC |
| | 452-27447524 (+) | CTGTCACGCGGGAGACCGGGGTTCGATTCCC |
| 345 | Ser_TGA_chr6: 27473 | GTAGTCGTGGCCGAGTGGTTAAGGCGATGGA |
| | 606-27473688 (−) | CTTGAAATCCATTGGGGTTTCCCCGCGCAGG |
| 346 | Gln_CTG_chr6: 27487 | AGGTTCCATGGTGTAATGGTTAGCACTCTGG |
| | 307-27487379 (+) | ACTCTGAATCCAGCGATCCGAGTTCAAATCT |
| 347 | Asp_GTC_chr6: 27551 | TCCTCGTTAGTATAGTGGTGAGTGTCCCCGTC |
| | 235-27551307 (−) | TGTCACGCGGGAGACCGGGGTTCGATTCCCC |
| 348 | Val_AAC_chr6: 27618 | GTTTCCGTAGTGTAGTGGTTATCACGTTCGCC |
| | 706-27618779 (−) | TAACACGCGAAAGGTCCCTGGATCAAAACCA |
| 349 | Ile_AAT_chr6: 276559 | CGGCCGGTTAGCTCAGTTGGTTAGAGCGTGG |
| | 66-27656040 (+) | TGCTAATAACGCCAAGGTCGCGGGTTCGATC |
| 350 | Gln_CTG_chr6: 27759 | GGCCCCATGGTGTAATGGTCAGCACTCTGGA |
| | 134-27759206 (−) | CTCTGAATCCAGCGATCCGAGTTCAAATCTC |
| 351 | Gln_TTG_chr6: 27763 | GGCCCCATGGTGTAATGGTTAGCACTCTGGA |
| | 639-27763711 (−) | CTTTGAATCCAGCGATCCGAGTTCAAATCTC |
| 352 | Ala_AGC_chr6: 28574 | TGGGGGTGTAGCTCAGTGGTAGAGCGCGTGC |
| | 932-28575004 (+) | TTAGCATGTACGAGGTCCCGGGTTCAATCCC |
| 353 | Ala_AGC_chr6: 28626 | GGGGATGTAGCTCAGTGGTAGAGCGCATGCT |
| | 013-28626085 (−) | TAGCATGCATGAGGTCCCGGGTTCGATCCCC |
| 354 | Ala_CGC_chr6: 28697 | AGGGGGTGTAGCTCAGTGGTAGAGCGCGTGC |
| | 091-28697163 (+) | TTCGCATGTACGAGGCCCCGGGTTCGACCCC |
| 355 | Ala_AGC_chr6: 28806 | GGGGGTGTAGCTCAGTGGTAGAGCGCGTGCT |
| | 220-28806292 (−) | TAGCATGCACGAGGCCCCGGGTTCAATCCCC |
| 356 | Ala_AGC_chr6: 28831 | GGGGGTGTAGCTCAGTGGTAGAGCGCGTGCT |
| | 461-28831533 (−) | TAGCATGCACGAGGCCCCGGGTTCAATCCCC |
| 357 | Leu_CAA_chr6: 28863 | GTCAGGATGGCCGAGTGGTCTAAGGCGCCAG |
| | 999-28864105 (−) | ACTCAAGCTAAGCTTCCTCCGCGGTGGGGAT |
| 358 | Leu_CAA_chr6: 28908 | TGTCAGGATGGCCGAGTGGTCTAAGGCGCCA |
| | 829-28908934 (+) | GACTCAAGCTTGGCTTCCTCGTGTTGAGGATT |
| 359 | Gln_CTG_chr6: 28909 | GGTTCCATGGTGTAATGGTTAGCACTCTGGA |
| | 377-28909449 (−) | CTCTGAATCCAGCGATCCGAGTTCAAATCTC |
| 360 | Leu_AAG_chr6: 2891 | GGTAGCGTGGCCGAGCGGTCTAAGGCGCTGG |
| | 1398-28911480 (−) | ATTAAGGCTCCAGTCTCTTCGGGGGCGTGGG |
| 361 | Met_CAT_chr6: 28912 | TGCCTCCTTAGCGCAGTAGGCAGCGCGTCAG |
| | 351-28912424 (+) | TCTCATAATCTGAAGGTCCTGAGTTCGAACCT |
| 362 | Lys_TTT_chr6: 28918 | AGCCCGGATAGCTCAGTCGGTAGAGCATCAG |
| | 805-28918878 (+) | ACTTTTAATCTGAGGGTCCAGGGTTCAAGTC |
| 363 | Met_CAT_chr6: 28921 | GCCTCCTTAGCGCAGTAGGCAGCGCGTCAGT |
| | 041-28921114 (−) | CTCATAATCTGAAGGTCCTGAGTTCGAACCT |
| 364 | Glu_CTC_chr6: 28949 | TTCCCTGGTGGTCTAGTGGTTAGGATTCGGCG |
| | 975-28950047 (+) | CTCTCACCGCCGCGGCCCGGGTTCGATTCCC |
| 365 | Leu_TAA_chr6: 14453 | CACCAGGATGGCCGAGTGGTTAAGGCGTTGG |
| | 7683-144537766 (+) | ACTTAAGATCCAATGGACATATGTCCGCGTG |
| 366 | Pro_AGG_chr7: 12842 | TGGCTCGTTGGTCTAGGGGTATGATTCTCGCT |
| | 3503-128423575 (+) | TAGGGTGCGAGAGGTCCCGGGTTCAAATCCC |
| 367 | Arg_CCT_chr7: 13902 | AGCCCCAGTGGCCTAATGGATAAGGCATTGG |
| | 5445-139025518 (+) | CCTCCTAAGCCAGGGATTGTGGGTTCGAGTC |
| 368 | Cys_GCA_chr7: 14938 | GGGGATATAGCTCAGGGGTAGAGCATTTGAC |
| | 8271-149388343 (−) | TGCAGATCAAGAGGTCCCCGGTTCAAATCCG |
| 369 | Tyr_GTA_chr8: 67025 | CCCTTCGATAGCTCAGCTGGTAGAGCGGAGG |
| | 601-67025694 (+) | ACTGTAGCTACTTCCTCAGCAGGAGACATCC |
| 370 | Tyr_GTA_chr8: 67026 | CCCTTCGATAGCTCAGCTGGTAGAGCGGAGG |
| | 222-67026311 (+) | ACTGTAGGCGCGCGCCCGTGGCCATCCTTAG |
| 371 | Ala_AGC_chr8: 67026 | TGGGGGATTAGCTCAAATGGTAGAGCGCTCG |
| | 423-67026496 (+) | CTTAGCATGCGAGAGGTAGCGGGATCGATGC |
| 372 | Ser_AGA_chr8: 96281 | GTAGTCGTGGCCGAGTGGTTAAGGCGATGGA |
| | 884-96281966 (−) | CTAGAAATCCATTGGGGTCTCCCCGCGCAGG |
| 373 | Met_CAT_chr8: 12416 | GCCTCGTTAGCGCAGTAGGTAGCGCGTCAGT |
| | 9469-124169542 (−) | CTCATAATCTGAAGGTCGTGAGTTCGATCCTC |
| 374 | Arg_TCT_chr9: 13110 | GGCTCTGTGGCGCAATGGATAGCGCATTGGA |
| | 2354-131102445 (−) | CTTCTAGCTGAGCCTAGTGTGGTCATTCAAA |
| 375 | Asn_GTT_chr10: 2251 | GTCTCTGTGGCGCAATCGGTTAGCGCGTTCG |
| | 8437-22518511 (−) | GCTGTTAACCGAAAGGTTGGTGGTTCGAGCC |
| 376 | Ser_TGA_chr10: 6952 | GGCAGCGATGGCCGAGTGGTTAAGGCGTTGG |
| | 4260-69524342 (+) | ACTTGAAATCCAATGGGGTCTCCCCGCGCAG |
| 377 | Val_TAC_chr11: 5931 | GGTTCCATAGTGTAGTGGTTATCACGTCTGCT |
| | 8101-59318174 (−) | TTACACGCAGAAGGTCCTGGGTTCGAGCCCC |
| 378 | Val_TAC_chr11: 5931 | GGTTCCATAGTGTAGCGGTTATCACGTCTGCT |
| | 8459-59318532 (−) | TTACACGCAGAAGGTCCTGGGTTCGAGCCCC |
| 379 | Arg_TCT_chr11: 5931 | TGGCTCTGTGGCGCAATGGATAGCGCATTGG |
| | 8766-59318852 (+) | ACTTCTAGATAGTTAGAGAAATTCAAAGGTT |
| 380 | Leu_TAA_chr11: 5931 | TACCAGAATGGCCGAGTGGTTAAGGCGTTGG |
| | 9227-59319310 (+) | ACTTAAGATCCAATGGATTCATATCCGCGTG |
| 381 | Lys_TTT_chr11: 5932 | GGCCCGGATAGCTCAGTCGGTAGAGCATCAG |
| | 3901-59323974 (+) | ACTTTTAATCTGAGGGTCCGGGGTTCAAGTC |
| 382 | Phe_GAA_chr11: 5932 | GCCGAAATAGCTCAGTTGGGAGAGCGTTAGA |
| | 4969-59325042 (−) | CTGAAGATCTAAAGGTCCCTGGTTCGATCCC |
| 383 | Lys_TTT_chr11: 5932 | GCCCGGATAGCTCAGTCGGTAGAGCATCAGA |
| | 7807-59327880 (−) | CTTTTAATCTGAGGGTCCAGGGTTCAAGTCCC |
| 384 | Phe_GAA_chr11: 5933 | GCCGAAATAGCTCAGTTGGGAGAGCGTTAGA |
| | 3852-59333925 (−) | CTGAAGATCTAAAGGTCCCTGGTTCAATCCC |
| 385 | Ser_GCT_chr11: 6611 | GGACGAGGTGGCCGAGTGGTTAAGGCGATG |
| | 5590-66115672 (+) | GACTGCTAATCCATTGTGCTTTGCACGCGTGG |
| 386 | Pro_TGG_chr11: 7594 | GGCTCGTTGGTCTAGGGGTATGATTCTCGGTT |
| | 6868-75946940 (−) | TGGGTCCGAGAGGTCCCGGGTTCAAATCCCG |
| 387 | Ser_CGA_chr12: 5658 | AGTCACGGTGGCCGAGTGGTTAAGGCGTTGG |
| | 4147-56584229 (+) | ACTCGAAATCCAATGGGGTTTCCCCGCACAG |
| 388 | Asp_GTC_chr12: 9889 | CTCCTCGTTAGTATAGTGGTTAGTATCCCCGC |
| | 7280-98897352 (+) | CTGTCACGCGGGAGACCGGGGTTCAATTCCC |
| 389 | Trp_CCA_chr12: 9889 | GGACCTCGTGGCGCAACGGTAGCGCGTCTGA |
| | 8029-98898101 (+) | CTCCAGATCAGAAGGCTGCGTGTTCGAATCA |
| 390 | Ala_TGC_chr12: 1254 | GGGGATGTAGCTCAGTGGTAGAGCGCATGCT |
| | 06300-125406372 (−) | TTGCATGTATGAGGCCCCGGGTTCGATCCCC |
| 391 | Phe_GAA_chr12: 1254 | GCCGAAATAGCTCAGTTGGGAGAGCGTTAGA |
| | 12388-125412461 (−) | CTGAAGATCTAAAGGTCCCTGGTTCGATCCC |
| 392 | Ala_TGC_chr12: 1254 | AGGGGATGTAGCTCAGTGGTAGAGCGCATGC |
| | 24511-125424583 (+) | TTTGCACGTATGAGGCCCCGGGTTCAATCCC |
| 393 | Asn_GTT_chr13: 3124 | GTCTCTGTGGCGCAATCGGTTAGCGCGTTCG |
| | 8100-31248174 (−) | GCTGTTAACCGAAAGGTTGGTGGTTCGAGCC |
| 394 | Glu_TTC_chr13: 4549 | TCCCACATGGTCTAGCGGTTAGGATTCCTGGT |
| | 2061-45492133 (−) | TTTCACCCAGGCGGCCCGGGTTCGACTCCCG |
| 395 | Thr_TGT_chr14: 2108 | GGCTCCATAGCTCAGGGGTTAGAGCGCTGGT |
| | 1948-21082021 (−) | CTTGTAAACCAGGGGTCGCGAGTTCAATTCT |
| 396 | Leu_TAG_chr14: 2109 | TGGTAGTGTGGCCGAGCGGTCTAAGGCGCTG |
| | 3528-21093610 (+) | GATTTAGGCTCCAGTCTCTTCGGGGGCGTGG |
| 397 | Thr_TGT_chr14: 2109 | GGCTCCATAGCTCAGGGGTTAGAGCACTGGT |
| | 9318-21099391 (−) | CTTGTAAACCAGGGGTCGCGAGTTCAAATCT |
| 398 | Pro_TGG_chr14: 2110 | TGGCTCGTTGGTCTAGTGGTATGATTCTCGCT |
| | 1164-21101236 (+) | TTGGGTGCGAGAGGTCCCGGGTTCAAATCCC |
| 399 | Tyr_GTA_chr14: 2113 | CCTTCGATAGCTCAGCTGGTAGAGCGGAGGA |
| | 1350-21131444 (−) | CTGTAGATTGTACAGACATTTGCGGACATCC |
| 400 | Thr_TGT_chr14: 2114 | AGGCCCTATAGCTCAGGGGTTAGAGCACTGG |
| | 9848-21149921 (+) | TCTTGTAAACCAGGGGTCGCGAGTTCAAATC |
| 401 | Tyr_GTA_chr14: 2115 | TCCTTCGATAGCTCAGCTGGTAGAGCGGAGG |
| | 1431-21151520 (+) | ACTGTAGTACTTAATGTGTGGTCATCCTTAGG |
| 402 | Pro_TGG_chr14: 2115 | TGGCTCGTTGGTCTAGGGGTATGATTCTCGCT |
| | 2174-21152246 (+) | TTGGGTGCGAGAGGTCCCGGGTTCAAATCCC |
| 403 | Lys_CTT_chr14: 5870 | GCCCGGCTAGCTCAGTCGGTAGAGCATGGGA |
| | 6612-58706685 (−) | CTCTTAATCCCAGGGTCGTGGGTTCGAGCCC |
| 404 | Ile_AAT_chr14: 10278 | CGGCCGGTTAGCTCAGTTGGTTAGAGCGTGG |
| | 3428-102783502 (+) | TGCTAATAACGCCAAGGTCGCGGGTTCGATC |
| 405 | Glu_TTC_chr15: 2632 | TCCCACATGGTCTAGCGGTTAGGATTCCTGGT |
| | 7380-26327452 (−) | TTTCACCCAGGCGGCCCGGGTTCGACTCCCG |
| 406 | Ser_GCT_chr15: 4088 | GACGAGGTGGCCGAGTGGTTAAGGCGATGG |
| | 6022-40886104 (−) | ACTGCTAATCCATTGTGCTCTGCACGCGTGG |
| 407 | His_GTG_chr15: 4549 | GCCGTGATCGTATAGTGGTTAGTACTCTGCGT |
| | 0803-45490875 (−) | TGTGGCCGCAGCAACCTCGGTTCGAATCCGA |
| 408 | His_GTG_chr15: 4549 | CGCCGTGATCGTATAGTGGTTAGTACTCTGC |
| | 3348-45493420 (+) | GTTGTGGCCGCAGCAACCTCGGTTCGAATCC |
| 409 | Gln_CTG_chr15: 6616 | GGTTCCATGGTGTAATGGTTAGCACTCTGGA |
| | 1399-66161471 (−) | CTCTGAATCCAGCGATCCGAGTTCAAATCTC |
| 410 | Lys_CTT_chr15: 7915 | TGCCCGGCTAGCTCAGTCGGTAGAGCATGGG |
| | 2903-79152976 (+) | ACTCTTAATCCCAGGGTCGTGGGTTCGAGCC |
| 411 | Arg_TCG_chr15: 8987 | GGGCCGCGTGGCCTAATGGATAAGGCGTCTG |
| | 8303-89878376 (+) | ACTTCGGATCAGAAGATTGCAGGTTCGAGTC |
| 412 | Gly_CCC_chr16: 6867 | GCGCCGCTGGTGTAGTGGTATCATGCAAGAT |
| | 35-686806 (−) | TCCCATTCTTGCGACCCGGGTTCGATTCCCGG |
| 413 | Arg_CCG_chr16: 3200 | GGGCCGCGTGGCCTAATGGATAAGGCGTCTG |
| | 674-3200747 (+) | ATTCCGGATCAGAAGATTGAGGGTTCGAGTC |
| 414 | Arg_CCT_chr16: 3202 | CGCCCCGGTGGCCTAATGGATAAGGCATTGG |
| | 900-3202973 (+) | CCTCCTAAGCCAGGGATTGTGGGTTCGAGTC |
| 415 | Lys_CTT_chr16: 3207 | GCCCGGCTAGCTCAGTCGGTAGAGCATGAGA |
| | 405-3207478 (−) | CCCTTAATCTCAGGGTCGTGGGTTCGAGCCC |
| 416 | Thr_CGT_chr16: 1437 | AGGCGCGGTGGCCAAGTGGTAAGGCGTCGGT |
| | 9749-14379821 (+) | CTCGTAAACCGAAGATCACGGGTTCGAACCC |
| 417 | Leu_TAG_chr16: 2220 | GGTAGCGTGGCCGAGTGGTCTAAGGCGCTGG |
| | 7031-22207113 (−) | ATTTAGGCTCCAGTCATTTCGATGGCGTGGGT |
| 418 | Leu_AAG_chr16: 223 | GGGTAGCGTGGCCGAGCGGTCTAAGGCGCTG |
| | 08460-22308542 (+) | GATTAAGGCTCCAGTCTCTTCGGGGGCGTGG |
| 419 | Leu_CAG_chr16: 5733 | AGTCAGGATGGCCGAGCGGTCTAAGGCGCTG |
| | 3862-57333945 (+) | CGTTCAGGTCGCAGTCTCCCCTGGAGGCGTG |
| 420 | Leu_CAG_chr16: 5733 | GTCAGGATGGCCGAGCGGTCTAAGGCGCTGC |
| | 4391-57334474 (−) | GTTCAGGTCGCAGTCTCCCCTGGAGGCGTGG |
| 421 | Met_CAT_chr16: 8741 | GCCTCGTTAGCGCAGTAGGCAGCGCGTCAGT |
| | 7627-87417700 (−) | CTCATAATCTGAAGGTCGTGAGTTCGAGCCT |
| 422 | Leu_TAG_chr17: 8023 | GGTAGCGTGGCCGAGCGGTCTAAGGCGCTGG |
| | 631-8023713 (−) | ATTTAGGCTCCAGTCTCTTCGGAGGCGTGGG |
| 423 | Arg_TCT_chr17: 8024 | TGGCTCTGTGGCGCAATGGATAGCGCATTGG |
| | 242-8024330 (+) | ACTTCTAGTGACGAATAGAGCAATTCAAAGG |
| 424 | Gly_GCC_chr17: 8029 | CGCATTGGTGGTTCAGTGGTAGAATTCTCGC |
| | 063-8029134 (+) | CTGCCACGCGGGAGGCCCGGGTTCGATTCCC |
| 425 | Ser_CGA_chr17: 8042 | GCTGTGATGGCCGAGTGGTTAAGGCGTTGGA |
| | 198-8042280 (−) | CTCGAAATCCAATGGGGTCTCCCCGCGCAGG |
| 426 | Thr_AGT_chr17: 8042 | GGCGCCGTGGCTTAGCTGGTTAAAGCGCCTG |
| | 769-8042843 (−) | TCTAGTAAACAGGAGATCCTGGGTTCGAATC |
| 427 | Trp_CCA_chr17: 8089 | CGACCTCGTGGCGCAACGGTAGCGCGTCTGA |
| | 675-8089747 (+) | CTCCAGATCAGAAGGTTGCGTGTTCAAATCA |
| 428 | Ser_GCT_chr17: 8090 | AGACGAGGTGGCCGAGTGGTTAAGGCGATG |
| | 183-8090265 (+) | GACTGCTAATCCATTGTGCTCTGCACGCGTG |
| 429 | Thr_AGT_chr17: 8090 | CGGCGCCGTGGCTTAGTTGGTTAAAGCGCCT |
| | 477-8090551 (+) | GTCTAGTAAACAGGAGATCCTGGGTTCGAAT |
| 430 | Trp_CCA_chr17: 8124 | GGCCTCGTGGCGCAACGGTAGCGCGTCTGAC |
| | 186-8124258 (−) | TCCAGATCAGAAGGTTGCGTGTTCAAATCAC |
| 431 | Gly_TCC_chr17: 8124 | AGCGTTGGTGGTATAGTGGTAAGCATAGCTG |
| | 865-8124937 (+) | CCTTCCAAGCAGTTGACCCGGGTTCGATTCCC |
| 432 | Asp_GTC_chr17: 8125 | TCCTCGTTAGTATAGTGGTGAGTATCCCCGCC |
| | 555-8125627 (−) | TGTCACGCGGGAGACCGGGGTTCGATTCCCC |
| 433 | Pro_CGG_chr17: 8126 | GGCTCGTTGGTCTAGGGGTATGATTCTCGCTT |
| | 150-8126222 (−) | CGGGTGCGAGAGGTCCCGGGTTCAAATCCCG |
| 434 | Thr_AGT_chr17: 8129 | GGCGCCGTGGCTTAGTTGGTTAAAGCGCCTG |
| | 552-8129626 (−) | TCTAGTAAACAGGAGATCCTGGGTTCGAATC |
| 435 | Ser_AGA_chr17: 8129 | GTAGTCGTGGCCGAGTGGTTAAGGCGATGGA |
| | 927-8130009 (−) | CTAGAAATCCATTGGGGTCTCCCCGCGCAGG |
| 436 | Trp_CCA_chr17: 1941 | TGACCTCGTGGCGCAATGGTAGCGCGTCTGA |
| | 1493-19411565 (+) | CTCCAGATCAGAAGGTTGCGTGTTCAAGTCA |
| 437 | Thr_CGT_chr17: 2987 | AGGCGCGGTGGCCAAGTGGTAAGGCGTCGGT |
| | 7092-29877164 (+) | CTCGTAAACCGAAGATCGCGGGTTCGAACCC |
| 438 | Cys_GCA_chr17: 3702 | AGGGGGTATAGCTCAGTGGTAGAGCATTTGA |
| | 3897-37023969 (+) | CTGCAGATCAAGAGGTCCCCGGTTCAAATCC |
| 439 | Cys_GCA_chr17: 3702 | GGGGGTATAGCTCAGTGGTAGAGCATTTGAC |
| | 5544-37025616 (−) | TGCAGATCAAGAGGTCCCTGGTTCAAATCCG |
| 440 | Cys_GCA_chr17: 3730 | GGGGGTATAGCTCAGTGGTAGAGCATTTGAC |
| | 9986-37310058 (−) | TGCAGATCAAGAGGTCCCCGGTTCAAATCCG |
| 441 | Gln_TTG_chr17: 4726 | AGGTCCCATGGTGTAATGGTTAGCACTCTGG |
| | 9889-47269961 (+) | ACTTTGAATCCAGCGATCCGAGTTCAAATCT |
| 442 | Arg_CCG_chr17: 6601 | GACCCAGTGGCCTAATGGATAAGGCATCAGC |
| | 6012-66016085 (−) | CTCCGGAGCTGGGGATTGTGGGTTCGAGTCC |
| 443 | Arg_CCT_chr17: 7303 | AGCCCCAGTGGCCTAATGGATAAGGCACTGG |
| | 0000-73030073 (+) | CCTCCTAAGCCAGGGATTGTGGGTTCGAGTC |
| 444 | Arg_CCT_chr17: 7303 | GCCCCAGTGGCCTAATGGATAAGGCACTGGC |
| | 0525-73030598 (−) | CTCCTAAGCCAGGGATTGTGGGTTCGAGTCC |
| 445 | Arg_TCG_chr17: 7303 | AGACCGCGTGGCCTAATGGATAAGGCGTCTG |
| | 1207-73031280 (+) | ACTTCGGATCAGAAGATTGAGGGTTCGAGTC |
| 446 | Asn_GTT_chr19: 1383 | CGTCTCTGTGGCGCAATCGGTTAGCGCGTTC |
| | 561-1383635 (+) | GGCTGTTAACCGAAAGGTTGGTGGTTCGAGC |
| 447 | Gly_TCC_chr19: 4724 | GGCGTTGGTGGTATAGTGGTTAGCATAGCTG |
| | 081-4724153 (+) | CCTTCCAAGCAGTTGACCCGGGTTCGATTCCC |
| 448 | Val_CAC_chr19: 4724 | GTTTCCGTAGTGTAGCGGTTATCACATTCGCC |
| | 646-4724719 (−) | TCACACGCGAAAGGTCCCCGGTTCGATCCCG |
| 449 | Thr_AGT_chr19: 3366 | TGGCGCCGTGGCTTAGTTGGTTAAAGCGCCT |
| | 7962-33668036 (+) | GTCTAGTAAACAGGAGATCCTGGGTTCGAAT |
| 450 | Ile_TAT_chr19: 39902 | GCTCCAGTGGCGCAATCGGTTAGCGCGCGGT |
| | 807-39902900 (−) | ACTTATATGACAGTGCGAGCGGAGCAATGCC |
| 451 | Gly_GCC_chr21: 1882 | GCATGGGTGGTTCAGTGGTAGAATTCTCGCC |
| | 7106-18827177 (−) | TGCCACGCGGGAGGCCCGGGTTCGATTCCCG |

### Non-Naturally Occurring Modification

A TREM, a TREM core fragment or a TREM fragment described herein comprises a non-naturally occurring modification, e.g., a modification described in any one of Tables 5-9. A non-naturally occurring modification can be made according to methods known in the art. Exemplary methods of making non-naturally occurring modifications are provided in Examples 4-7.

In an embodiment, a non-naturally occurring modification is a modification that a cell, e.g., a human cell, does not make on an endogenous tRNA.

In an embodiment, a non-naturally occurring modification is a modification that a cell, e.g., a human cell, can make on an endogenous tRNA, but wherein such modification is in a location in which it does not occur on a native tRNA. In an embodiment, the non-naturally occurring modification is in a domain, linker or arm which does not have such modification in nature. In an embodiment, the non-naturally occurring modification is at a position within a domain, linker or arm, which does not have such modification in nature. In an embodiment, the non-naturally occurring modification is on a nucleotide which does not have such modification in nature. In an embodiment, the non-naturally occurring modification is on a nucleotide at a position within a domain, linker or arm, which does not have such modification in nature.

In an embodiment, a TREM, a TREM core fragment or a TREM fragment described herein comprises a non-naturally occurring modification provided in Table 5, or a combination thereof.

TABLE 5
Exemplary non-naturally occurring modifications
Modification

7-deaza-adenosine
N1-methyl-adenosine
N6, N6 (dimethyl)adenine
N6-cis-hydroxy-isopentenyl-adenosine
thio-adenosine
2-(amino)adenine
2-(aminopropyl)adenine
2-(methylthio) N6 (isopentenyl)adenine
2-(alkyl)adenine
2-(aminoalkyl)adenine
2-(aminopropyl)adenine
2-(halo)adenine
2-(propyl)adenine
2′-azido-2′-deoxy-adenosine
2′-Deoxy-2′-alpha-aminoadenosine
2′-Deoxy-2′-alpha-azidoadenosine
6-(alkyl)adenine
6-(methyl)adenine
6-(alkyl)adenine
6-(methyl)adenine
7-(deaza)adenine
8-(alkenyl)adenine
8-(alkynyl)adenine
8-(amino)adenine
8-(thioalkyl)adenine
8-(alkenyl)adenine
8-(alkyl)adenine
8-(alkynyl)adenine
8-(amino)adenine
8-(halo)adenine
8-(hydroxyl)adenine
8-(thioalkyl)adenine
8-(thiol)adenine
8-azido-adenosine
azaadenine
deazaadenine
N6-(methyl)adenine
N6-(isopentyl)adenine
7-deaza-8-aza-adenosine
7-methyladenine
1-deazaadenosine
2′-Fluoro-N6-Bz-deoxyadenosine
2′-OMe-2-Amino-adenosine
2′O-methyl-N6-Bz-deoxyadenosine
2′alpha-ethynyladenosine
2-aminoadenine
2-Aminoadenosine
2-Amino-adenosine
2′-alpha-Trifluoromethyladenosine
2-Azidoadenosine
2′-beta-Ethynyladenosine
2-Bromoadenosine
2′-beta-Trifluoromethyladenosine
2-Chloroadenosine
2′-Deoxy-2′,2′-difluoroadenosine
2′-Deoxy-2′-alpha-mercaptoadenosine
2′-Deoxy-2′-alpha-thiomethoxyadenosine
2′-Deoxy-2′-beta-aminoadenosine
2′-Deoxy-2′-beta-azidoadenosine
2′-Deoxy-2′-beta-bromoadenosine
2′-Deoxy-2′-beta-chloroadenosine
2′-Deoxy-2′-beta-fluoroadenosine
2′-Deoxy-2′-beta-iodoadenosine
2′-Deoxy-2′-beta-mercaptoadenosine
2′-Deoxy-2′-beta-thiomethoxyadenosine
2-Fluoroadenosine
2-Iodoadenosine
2-Mercaptoadenosine
2-methoxy-adenine
2-methylthio-adenine
2-Trifluoromethyladenosine
3-Deaza-3-bromoadenosine
3-Deaza-3-chloroadenosine
3-Deaza-3-fluoroadenosine
3-Deaza-3-iodoadenosine
3-Deazaadenosine
4′-Azidoadenosine
4′-Carbocyclic adenosine
4′-Ethynyladenosine
5′-Homo-adenosine
8-Aza-adenosine
8-bromo-adenosine
8-Trifluoromethyladenosine
9-Deazaadenosine
2-aminopurine
7-deaza-2,6-diaminopurine
7-deaza-8-aza-2,6-diaminopurine
7-deaza-8-aza-2-aminopurine
2,6-diaminopurine
7-deaza-8-aza-adenine, 7-deaza-2-aminopurine
4-methylcytidine
5-aza-cytidine
Pseudo-iso-cytidine
pyrrolo-cytidine
alpha-thio-cytidine
2-(thio)cytosine
2′-Amino-2′-deoxy-cytosine
2′-Azido-2′-deoxy-cytosine
2′-Deoxy-2′-alpha-aminocytidine
2′-Deoxy-2′-alpha-azidocytidine
3 (deaza) 5 (aza)cytosine
3 (methyl)cytosine
3-(alkyl)cytosine
3-(deaza) 5 (aza)cytosine
3-(methyl)cytidine
4,2′-O-dimethylcytidine
5 (halo)cytosine
5 (methyl)cytosine
5 (propynyl)cytosine
5 (trifluoromethyl)cytosine
5-(alkyl)cytosine
5-(alkynyl)cytosine
5-(halo)cytosine
5-(propynyl)cytosine
5-(trifluoromethyl)cytosine
5-bromo-cytidine
5-iodo-cytidine
5-propynyl cytosine
6-(azo)cytosine
6-aza-cytidine
aza cytosine
deaza cytosine
N4 (acetyl)cytosine
1-methyl-1-deaza-pseudoisocytidine
1-methyl-pseudoisocytidine
2-methoxy-5-methyl-cytidine
2-methoxy-cytidine
2-thio-5-methyl-cytidine
4-methoxy-1-methyl-pseudoisocytidine
4-methoxy-pseudoisocytidine
4-thio-1-methyl-1-deaza-pseudoisocytidine
4-thio-1-methyl-pseudoisocytidine
4-thio-pseudoisocytidine
5-aza-zebularine
5-methyl-zebularine
pyrrolo-pseudoisocytidine
zebularine
(E)-5-(2-Bromo-vinyl)cytidine
2,2′-anhydro-cytidine
2′-Fluor-N4-Bz-cytidine
2′-Fluoro-N4-Acetyl-cytidine
2′-O-Methyl-N4-Acetyl-cytidine
2′-O-methyl-N4-Bz-cytidine
2′-a-Ethynylcytidine
2′-a-Trifluoromethylcytidine
2′-b-Ethynylcytidine
2′-b-Trifluoromethylcytidine
2′-Deoxy-2′,2′-difluorocytidine
2′-Deoxy-2′-alpha-mercaptocytidine
2′-Deoxy-2′-alpha-thiomethoxycytidine
2′-Deoxy-2′-betab-aminocytidine
2′-Deoxy-2′-beta-azidocytidine
2′-Deoxy-2′-beta-bromocytidine
2′-Deoxy-2′-beta-chlorocytidine
2′-Deoxy-2′-beta-fluorocytidine
2′-Deoxy-2′-beta-iodocytidine
2′-Deoxy-2′-beta-mercaptocytidine
2′-Deoxy-2′-beta-thiomethoxycytidine TP
2′-O-Methyl-5-(1-propynyl)cytidine
3′-Ethynylcytidine
4′-Azidocytidine
4′-Carbocyclic cytidine
4′-Ethynylcytidine
5-(1-Propynyl)ara-cytidine
5-(2-Chloro-phenyl)-2-thiocytidine
5-(4-Amino-phenyl)-2-thiocytidine
5-Aminoallyl-cytosine
5-Cyanocytidine
5-Ethynylara-cytidine
5-Ethynylcytidine
5′-Homo-cytidine
5-Methoxycytidine
5-Trifluoromethyl-Cytidine
N4-Amino-cytidine
N4-Benzoyl-cytidine
pseudoisocytidine
6-thio-guanosine
7-deaza-guanosine
8-oxo-guanosine
N1-methyl-guanosine
alpha-thio-guanosine
2-(propyl)guanine
2-(alkyl)guanine
2′-Amino-2′-deoxy-guanosine
2′-Azido-2′-deoxy-guanosine
2′-Deoxy-2′-alpha-aminoguanosine
2′-Deoxy-2′-alpha-azidoguanosine
6-(methyl)guanine
6-(alkyl)guanine
6-(methyl)guanine
6-methyl-guanosine
7-(alkyl)guanine
7-(deaza)guanine
7-(methyl)guanine
7-(alkyl)guanine
7-(deaza)guanine
7-(methyl)guanine
8-(alkyl)guanine
8-(alkynyl)guanine
8-(halo)guanine
8-(thioalkyl)guanine
8-(alkenyl)guanine
8-(alkyl)guanine
8-(alkynyl)guanine
8-(amino)guanine
8-(halo)guanine
8-(hydroxyl)guanine
8-(thioalkyl)guanine
8-(thiol)guanine
azaguanine
deaza guanine
N (methyl)guanine
N-(methyl)guanine
1-methyl-6-thio-guanosine
6-methoxy-guanosine
6-thio-7-deaza-8-aza-guanosine
6-thio-7-deaza-guanosine
6-thio-7-methyl-guanosine
7-deaza-8-aza-guanosine
7-methyl-8-oxo-guanosine
N2,N2-dimethyl-6-thio-guanosine
N2-methyl-6-thio-guanosine
1-Me-guanosine
2′Fluoro-N2-isobutyl-guanosine
2′O-methyl-N2-isobutyl-guanosine
2′-alpha-Ethynylguanosine
2′-alpha-Trifluoromethylguanosine
2′-beta-Ethynylguanosine
2′-beta-Trifluoromethylguanosine
2′-Deoxy-2′,2′-difluoroguanosine
2′-Deoxy-2′-alpha-mercaptoguanosine
2′-Deoxy-2′-alpha-thiomethoxyguanosine
2′-Deoxy-2′-beta-aminoguanosine
8-(alkyl)guanine
2′-Deoxy-2′-beta-azidoguanosine
2′-Deoxy-2′-beta-bromoguanosine
2′-Deoxy-2′-beta-chloroguanosine
2′-Deoxy-2′-beta-fluoroguanosine
2′-Deoxy-2′-beta-iodoguanosine
2′-Deoxy-2′-beta-mercaptoguanosine
2′-Deoxy-2′-beta-thiomethoxyguanosine
4′-Azidoguanosine
4′-Carbocyclic guanosine
4′-Ethynylguanosine
5′-Homo-guanosine
8-bromo-guanosine
9-Deazaguanosine
N2-isobutyl-guanosine
7-methylinosine
allyamino-thymidine
aza thymidine
deaza thymidine
deoxy-thymidine
5-propynyl uracil
alpha-thio-uridine
1-(aminoalkylamino-carbonylethylenyl)-2(thio)-pseudouracil
1-(aminoalkylaminocarbonylethylenyl)-2,4-(dithio)psuedouracil
1-(aminoalkylaminocarbonylethylenyl)-4(thio)pseudouracil
1-(aminoalkylaminocarbonylethylenyl)-pseudouracil
1-( aminocarbonylethylenyl)-2(thio)-pseudouracil
1-( aminocarbonylethylenyl)-2,4-( dithio)pseudouracil
1-(aminocarbonylethylenyl)-4(thio)pseudouracil
1-(aminocarbonylethylenyl)-pseudouracil
1-substituted 2-(thio)-pseudouracil
1-substituted 2,4-(dithio)pseudouracil
1-substituted 4 (thio)pseudouracil
1-substituted pseudouracil
1-(aminoalkylamino-carbonylethylenyl)-2(thio)-pseudouracil
1-Methyl-3-(3-amino-3-carboxypropyl)pseudouridine
1-Methyl-3-(3-amino-3-carboxyproovl)pseudo-Uradine
1-Methyl-pseudo-UTP
2 (thio)pseudouracil
2′ deoxy uridine
2′ fluorouridine
2-(thio)uracil
2,4-(dithio)pseudouracil
2′-methyl, 2′-amino, 2′azido, 2′fluro-guanosine
2′-Amino-2′-deoxy-uridine
2′-Azido-2′-deoxy-uridine
2′-Azido-deoxyuridine
2′-O-methylpseudouridine
2′ deoxyuridine
2′ fluorouridine
2′-Deoxy-2′-alpha-aminouridine TP
2′-Deoxy-2′-alpha-azidouridine TP
2-methylpseudouridine
3-(3 amino-3-carboxypropyl)uracil
4-(thio)pseudouracil
4-(thio )pseudouracil
4-(thio)uracil
4-thiouracil
5-(1,3-diazole-1-alkyl)uracil
5-(2-aminopropyl)uracil
5-(aminoalkyl)uracil
5-(dimethylaminoalkyl)uracil
5-(guanidiniumalkyl)uracil
5-(methoxycarbonylmethyl)-2-(thio)uracil
5-(methoxycarbonyl-methyl)uracil
5-(methyl)-2-(thio)uracil
5-(methyl)-2,4-(dithio)uracil
5 (methyl) 4 (thio)uracil
5 (methylaminomethyl)-2 (thio)uracil
5 (methylaminomethyl)-2,4 (dithio)uracil
5 (methylaminomethyl)-4 (thio)uracil
5 (propynyl)uracil
5 (trifluoromethyl)uracil
5-(2-aminopropyl)uracil
5-(alkyl)-2-(thio)pseudouracil
5-(alkyl)-2,4 (dithio)pseudouracil
5-(alkyl)-4 (thio)pseudouracil
5-(alkyl)pseudouracil
5-(alkyl)uracil
5-(alkynyl)uracil
5-(allylamino)uracil
5-(cyanoalkyl)uracil
5-(dialkylaminoalkyl)uracil
5-(dimethylaminoalkyl)uracil
5-(guanidiniumalkyl)uracil
5-(halo)uracil
5-(1,3-diazole-1-alkyl)uracil
5-(methoxy)uracil
5-(methoxycarbonylmethyl)-2-(thio)uracil
5-(methoxycarbonyl-methyl)uracil
5-(methyl) 2(thio)uracil
5-(methyl) 2,4 (dithio )uracil
5-(methyl) 4 (thio)uracil
5-(methyl)-2-(thio)pseudouracil
5-(methyl)-2,4 (dithio)pseudouracil
5-(methyl)-4 (thio)pseudouracil
5-(methyl)pseudouracil
5-(methylaminomethyl)-2 (thio)uracil
5-(methylaminomethyl)-2,4(dithio )uracil
5-(methylaminomethyl)-4-(thio)uracil
5-(propynyl)uracil
5-(trifluoromethyl)uracil
5-aminoallyl-uridine
5-bromo-uridine
5-iodo-uridine
5-uracil
6 (azo)uracil
6-(azo)uracil
6-aza-uridine
allyamino-uracil
aza uracil
deaza uracil
N3 (methyl)uracil
Pseudo-uridine-1-2-ethanoic acid
pseudouracil
4-Thio-pseudouridine
1-carboxymethyl-pseudouridine
1-methyl-1-deaza-pseudouridine
1-propynyl-uridine
1-taurinomethyl-1-methyl-uridine
1-taurinomethyl-4-thio-uridine
1-taurinomethyl-pseudouridine
2-methoxy-4-thio-pseudouridine
2-thio-1-methyl-1-deaza-pseudouridine
2-thio-1-methyl-pseudouridine
2-thio-5-aza-uridine
2-thio-dihydropseudouridine
2-thio-dihydrouridine
2-thio-pseudouridine
4-methoxy-2-thio-pseudouridine
4-methoxy-pseudouridine
4-thio-1-methyl-pseudouridine
4-thio-pseudouridine
5-aza-uridine
dihydropseudouridine
(±)1-(2-Hydroxypropyl)pseudouridine
(2R)-1-(2-Hydroxypropyl)pseudouridine
(2S)-1-(2-Hydroxypropyl)pseudouridine
(E)-5-(2-Bromo-vinyl)ara-uridine
(E)-5-(2-Bromo-vinyl)uridine
(Z)-5-(2-Bromo-vinyl)ara-uridine
(Z)-5-(2-Bromo-vinyl)uridine
1-(2,2,2-Trifluoroethyl)-pseudouridine
1-(2,2,3,3,3-Pentafluoropropyl)pseudouridine
1-(2,2-Diethoxyethyl)pseudouridine
1-(2,4,6-Trimethylbenzyl)pseudouridine
1-(2,4,6-Trimethyl-benzyl)pseudo-uridineuridine
1-(2,4,6-Trimethyl-phenyl)pseudo-uridine
1-(2-Amino-2-carboxyethyl)pseudo-uridine
1-(2-Amino-ethyl)pseudouridine
1-(2-Hydroxyethyl)pseudouridine
1-(2-Methoxyethyl)pseudouridine
1-(3,4-Bis-trifluoromethoxybenzyl)pseudouridine
1-(3,4-Dimethoxybenzyl)pseudouridine
1-(3-Amino-3-carboxypropyl)pseudo-uridine
1-(3-Amino-propyl)pseudouridine
1-(3-Cyclopropyl-prop-2-ynyl)pseudouridine TP
1-(4-Amino-4-carboxybutyl)pseudouridine
1-(4-Amino-benzyl)pseudouridine
1-(4-Amino-butyl)pseudouridine
1-(4-Amino-phenyl)pseudouridine
1-(4-Azidobenzyl)pseudouridine
1-(4-Bromobenzyl)pseudouridine
1-(4-Chlorobenzyl)pseudouridine
1-(4-Fluorobenzyl)pseudouridin
1-(4-Iodobenzyl)pseudouridine
1-(4-Methanesulfonylbenzyl)pseudouridine
1-(4-Methoxybenzyl)pseudouridine
1-(4-Methoxy-benzyl)pseudouridine
1-(4-Methoxy-phenyl)pseudouridine
1-(4-Methylbenzyl)pseudouridine
1-(4-Methyl-benzyl)pseudouridine
1-(4-Nitrobenzyl)pseudouridine
1-(4-Nitro-benzyl)pseudouridine
1( 4-Nitro-phenyl)pseudouridine
1-(4-Thiomethoxybenzyl)pseudouridine
1-(4-Trifluoromethoxybenzyl)pseudouridine
1-(4-Trifluoromethylbenzyl)pseudouridine
1-(5-Amino-pentyl)pseudouridine
1-(6-Amino-hexyl)pseudouridine
1,6-Dimethyl-pseudouridine
1-[3-(2-{2-[2-(2-Aminoethoxy)-ethoxy]-ethoxy }-ethoxy)-
propionyl } pseudouridine
1-{3-[2-(2-Aminoethoxy)-ethoxy]-propionyl } pseudouridine
1-Acetylpseudouridine
1-Alkyl-6-(1-propynyl)-pseudo-uridine
1-Alkyl-6-(2-propynyl)-pseudo-uridine
1-Alkyl-6-allyl-pseudo-uridine
1-Alkyl-6-ethynyl-pseudo-uridine
1-Alkyl-6-homoallyl-pseudo-uridine
1-Alkyl-6-vinyl-pseudo-uridine
1-Allylpseudouridine
1-Aminomethyl-pseudo-uridine
1-Benzoylpseudouridine
1-Benzyloxymethylpseudouridine
1-Benzyl-pseudo-uridine
1-Biotinyl-PEG2-pseudouridine
1-Biotinylpseudouridine
1-Butyl-pseudo-uridine
1-Cyanomethylpseudouridine
1-Cyclobutylmethyl-pseudo-uridine
1-Cyclobutyl-pseudo-uridine
1-Cycloheptylmethyl-pseudo-uridine
1-Cycloheptyl-pseudo-uridine
1-Cyclohexylmethyl-pseudo-uridine
1-Cyclohexyl-pseudo-uridine
1-Cyclooctylmethyl-pseudo-uridine
1-Cyclooctyl-pseudo-uridine
1-Cyclopentylmethyl-pseudo-uridine
1-Cyclopentyl-pseudo-uridine
1-Cyclopropylmethyl-pseudo-uridine
1-Cyclopropyl-pseudo-uridine
1-Ethyl-pseudo-uridine
1-Hexyl-pseudo-uridine
1-Homoallylpseudouridine
1-Hydroxymethylpseudouridine
1-iso-propyl-pseudo-uridine
1-Me-2-thio-pseudo-uridine
1-Me-4-thio-pseudo-uridine
1-Me-alpha-thio-pseudo-uridine
1-Methanesulfonylmethylpseudouridine
1-Methoxymethylpseudouridine uridine
1-Methyl-6-(2,2,2-Trifluoroethyl)pseudo-uridine
1-Methyl-6-(4-morpholino )-pseudo-uridine
1-Methyl-6-(4-thiomorpholino)-pseudo-uridine
1-Methyl-6-(substituted phenyl)pseudo-uridine
1-Methyl-6-amino-pseudo-uridine
1-Methyl-6-azido-pseudo-uridine
1-Methyl-6-bromo-pseudo-uridine
1-Methyl-6-butyl-pseudo-uridine
1-Methyl-6-chloro-pseudo-uridine
1-Methyl-6-cyano-pseudo-uridine
1-Methyl-6-dimethylamino-pseudo-uridine
1-Methyl-6-ethoxy-pseudo-uridine
1-Methyl-6-ethylcarboxylate-pseudo-uridine
1-Methyl-6-ethyl-pseudo-uridine
1-Methyl-6-fluoro-pseudo-uridine
1-Methyl-6-formyl-pseudo-uridine
1-Methyl-6-hydroxyamino-pseudo-uridine
1-Methyl-6-hydroxy-pseudo-uridine
1-Methyl-6-iodo-pseudo-uridine
1-Methyl-6-iso-propyl-pseudo-uridine
1-Methyl-6-methoxy-pseudo-uridine
1-Methyl-6-methylamino-pseudo-uridine
1-Methyl-6-phenyl-pseudo-uridine
1-Methyl-6-propyl-pseudo-uridine
1-Methyl-6-tert-butyl-pseudo-uridine
1-Methyl-6-trifluoromethoxy-pseudo-uridine
1-Methyl-6-trifluoromethyl-pseudo-uridine
1-Morpholinomethylpseudouridine
1-Pentyl-pseudo-uridineuridine
1-Phenyl-pseudo-uridine
1-Pivaloylpseudouridine
1-Propargylpseudouridine
1-Propyl-pseudo-uridine
1-propynyl-pseudouridine
1-p-tolyl-pseudo-uridine
1-tert-Butyl-pseudo-uridine
1-Thiomethoxymethylpseudouridine
1-Thiomorpholinomethylpseudouridine
1-Trifluoroacetylpseudouridine
1-Trifluoromethyl-pseudouridine
1-Vinylpseudouridine
2,2′-anhydro-uridine
2′-bromo-deoxyuridine
2′-F-5-Methyl-2′-deoxy-uridine
2′-OMe-5-Me-uridine
2′-OMe-pseudouridine
2′-alpha-Ethynyluridine
2′-alpha-Trifluoromethyluridine
2′-beta-Ethynyluridine
2′-beta-Trifluoromethyluridiner
2′-Deoxy-2′,2′-difluorouridine
2′-Deoxy-2′-a-mercaptouridin
2′-Deoxy-2′-alpha-thiomethoxyuridine
2′-Deoxy-2′-beta-aminouridine
2′-Deoxy-2′-beta-azidouridine
2′-Deoxy-2′-beta-bromouridine
2′-Deoxy-2′-beta-chlorouridine
2′-Deoxy-2′-beta-fluorouridine
2′-Deoxy-2′-beta-iodouridine
2′-Deoxy-2′-beta-mercaptouridine
2′-Deoxy-2′-beta-thiomethoxyuridine
2-methoxy-4-thio-uridine
2-methoxyuridine
2′-O-Methyl-5-(1-propynyl)uridine
3-Alkyl-pseudo-uridine
4′-Azidouridine
4′-Carbocyclic uridine
4′-Ethynyluridine
5-(1-Propynyl)ara-uridine
5-(2-Furanyl)uridine
5-Cyanouridine
5-Dimethylaminouridine
5′-Homo-uridine
5-iodo-2′-fluoro-deoxyuridine
5-Phenylethynyluridine
5-Trideuteromethyl-6-deuterouridine
5-Trifluoromethyl-Uridine
5-Vinylarauridine
6-(2,2,2-Trifluoroethyl)-pseudo-uridine
6-(4-Morpholino)-pseudo-uridine
6-(4-Thiomorpholino)-pseudo-uridine
6-(Substituted-Phenyl)-pseudo-uridine
6-Amino-pseudo-uridine
6-Azido-pseudo-uridine
6-Bromo-pseudo-uridine
6-Butyl-pseudo-uridine
6-Chloro-pseudo-uridine
6-Cyano-pseudo-uridine
6-Dimethylamino-pseudo-uridine
6-Ethoxy-pseudo-uridine
6-Ethylcarboxylate-pseudo-uridine
6-Ethyl-pseudo-uridine
6-Fluoro-pseudo-uridine
6-Formyl-pseudo-uridine
6-Hydroxyamino-pseudo-uridine
6-Hydroxy-pseudo-uridine
6-Iodo-pseudo-uridine
6-iso-Propyl-pseudo-uridine
6-Methoxy-pseudo-uridine
6-Methylamino-pseudo-uridine
6-Methyl-pseudo-uridine
6-Phenyl-pseudo-uridine
6-Phenyl-pseudo-uridine
6-Propyl-pseudo-uridine
6-tert-Butyl-pseudo-uridine
6-Trifluoromethoxy-pseudo-uridine
6-Trifluoromethyl-pseudo-uridine
alpha-thio-pseudo-uridine
Pseudouridine 1-(4-methylbenzenesulfonic acid)
Pseudouridine 1-(4-methylbenzoic acid)TP
Pseudouridine 1-[3-(2-ethoxy)]propionic acid
Pseudouridine 1-[3-{2-(2-[2-(2-ethoxy)-ethoxy]-ethoxy )-
ethoxy}]propionic acid
Pseudouridine 1-[3-{2-(2-[2-{2(2-etho)-ethoxy]-ethoxy )-
ethoxy}]propionic acid
Pseudouridine 1-[3-{2-(2-[2-ethoxy ]-ethoxy)-ethoxy}]propionic acid
Pseudouridine 1-[3-{2-(2-ethoxy)-ethoxy}] propionic acid
Pseudouridine 1-methylphosphonic acid
Pseudouridine TP 1-methylphosphonic acid diethyl ester
Pseudo-uridine-N1-3-butanoic acid
Pseudo-uridine-N1-4-butanoic acid
Pseudo-uridine-N 1-5-pentanoic acid
Pseudo-uridine-N1-6-hexanoic acid
Pseudo-uridine-N1-7-heptanoic acid
Pseudo-uridine-N1-methyl-p-benzoic acid
Pseudo-uridine-N1-p-benzoic acid

In an embodiment, a TREM, a TREM core fragment or a TREM fragment described herein comprises a modification provided in Table 6, or a combination thereof. The modifications provided in Table 6 occur naturally in RNAs, and are used herein on a synthetic TREM, a TREM core fragment or a TREM fragment at a position that does not occur in nature.

TABLE 6
Additional exemplary modifications

| Modification | Modification |
|---|---|
| 2-methylthio-N6-(cis- | 2-thiocytidine |
| hvdroxvisopentenvl)adenosine | 3-methylcytidine |
| 2-methylthio-N6-methyladenosine | 5-formylcytidine |
| 2-methylthio-N6-threonyl | 5-hydroxymethylcytidine |
| carbamoyladenosine | 5-methylcytidine |
| N6-glycinylcarbamoyladenosine | N4-acetylcytidine |
| N6-isopentenyladenosine | 2′-O-methylcytidine |
| N6-methyladenosine | 2′-O-methylcytidine |
| N6-threonylcarbamoyladenosine | 5,2′-O-dimethylcytidine |
| 1,2′-O-dimethyladenosine | 5-formyl-2′-O-methylcytidine |
| 1-methyladenosine | lysidine |
| 2′-O-methyladenosine | N4,2′-O-dimethy lcytidine |
| 2′-O-ribosyladenosine (phosphate) | N4-acetyl-2′-O-methylcytidine |
| 2-methyladenosine | N4-methylcytidine |
| 2-methylthio-N6 isopentenyladenosine | N4,N4-Dimethyl-2′-OMe-Cytidine |
| 2-methylthio-N6-hydroxynorvalyl | 7-methylguanosine |
| carbamoyladenosine | N2,2′-O-dimethylguanosine |
| 2′-O-methyladenosine | N2-methylguanosine |
| 2′-O-ribosyladenosine (phosphate) | wyosme |
| isopenteny ladenosine | 1,2′-O-dimethylguanosine |
| N6-(cis-hydroxyisopentenyl)adenosine | 1-methylguanosine |
| N6,2′-O-dimethyladenosine | 2′-O-methylguanosine |
| N6,2′-O-dimethyladenosine | 2′-O-ribosylguanosine (phosphate) |
| N6,N6,2′-O-trimethyladenosine | 2′-O-methylguanosine |
| N6,N6-dimethyladenosine | 2′-O-ribosylguanosine (phosphate) |
| N6-acetyladenosine | 1-methyl-pseudouridine |
| N6-hydroxynorvalylcarbamoyladenosine | 2′-O-methyluridine |
| N6-methyl-N6- | 2′-O-methylpseudouridine |
| threonylcarbamoyladenosine | 2′-O-methyluridine |
| 2-methyladenosine | 2-thio-2′-O-methyluridine |
| 2-methylthio-N⁶-isopentenyladenosine | 3-(3-amino-3-carboxypropyl)uridine |
| 7-aminomethyl-7-deazaguanosine | 3,2′-0-dimethyluridine |
| 7-cyano-7-deazaguanosine | 3-Methyl-pseudo-Uridine |
| archaeosine | 4-thiouridine |
| methylwyosine | 5-(carboxyhydroxymethyl)uridine |
| N2,7-dimethylguanosine | 5-(carboxyhydroxymethyl)uridine methyl |
| N2,N2,2′-O-trimethylguanosine | ester |
| N2,N2,7-trimethylguanosine | 5,2′-O-dimethyluridine |
| N2,N2-dimethylguanosine | 5,6-dihydro-uridine |
| N2, 7,2′-O-trimethylguanosine | 5-aminomethy1-2-thiouridine |
| 1-methylinosine | 5-carbamoylmethyl-2′-0-methyluridine |
| mosme | 5-carbamoylmethyluridine |
| 1,2′-O-dimethylinosine | 5-carboxyhydroxymethyluridine |
| 2′-O-methylinosine | 5-carboxyhydroxymethyluridine methyl |
| 2′-O-methylinosine | ester |
| epoxyqueuosine | 5-carboxymethylaminomethyl-2′-O- |
| galactosyl-queuosine | methyluridine |
| mannosyl-queuosine | 5-carboxymethylaminomethyl-2- |
| 2′-O-methyluridine | thiouridine |
| 2-thiouridine | 5-carboxymethylaminomethyl-2- |
| 3-methyluridine | thiouridine |
| 5-carboxymethyluridine | 5-carboxymethylaminomethyluridine |
| 5-hydroxyuridine | 5-carboxymethylaminomethyluridine |
| 5-methyluridine | 5-Carbamoylmethyluridine |
| 5-taurinomethyl-2-thiouridine | 5-methoxycarbonylmethyl-2′-O- |
| 5-taurinomethyluridine | methyluridine |
| dihydrouridine | 5-methoxycarbonylmethy 1-2-thiouridine |
| pseudouridine | 5-methoxycarbonylmethyluridine |
| (3-(3-amino-3-carboxypropyl)uridine | 5-methoxyuridine |
| 1-methyl-3-(3-amino-5- | 5-(iso-Pentenylaminomethyl)-2- |
| carboxypropyl)pseudouridine | thiouridine |
| 1-methylpseduouridine | 5-(iso-Pentenylaminomethyl)-2′-O- |
| 5-methyl-2-thiouridine | methyluridine |
| 5-methylaminomethyl-2-selenouridine | 5-(iso-Pentenylaminomethyl)uridine |
| 5-methylaminomethyl-2-thiouridine | wybutosine |
| 5-methylaminomethyluridine | hydroxywybutosine |
| 5-Methyldihydrouridine | isowyosme |
| 5-Oxyacetic acid- Uridine | peroxywybutosine |
| 5-Oxyacetic acid-methyl ester-Uridin | undermodified hydroxywybutosine |
| Nl-methyl-pseudo-uridine | 4-demethylwyosine |
| uridine 5-oxyacetic acid | altriol |
| uridine 5-oxyacetic acid methyl ester | |
| 3-(3-Amino-3-carboxypropyl)-Uridine | |

In an embodiment, a TREM, a TREM core fragment or a TREM fragment described herein comprises a non-naturally occurring modification provided in Table 7, or a combination thereof.

TABLE 7
Additional exemplary non-naturally occurring modifications

| Modification | Modification |
|---|---|
| 2,6-(diamino)purine | 2′-fluoro-modified bases |
| 1-(aza)-2-(thio)-3-(aza)-phenoxazin-1-yl | 2′-O-methyl-ribose |
| 1,3-(diaza)-2-(oxo)-phenthiazin-1-yl | 2-oxo-7-aminopyridopyrimidin-3-yl |
| 1,3-(diaza)-2-(oxo)-phenoxazin-1-yl | 2-oxo-pyridopyrimidine-3-yl |
| 1,3,5-(triaza)-2,6-(dioxa)-naphthalene | 2-pyridinone |
| 2 (amino)purine | 3 nitropyrrole |
| 2,4,5-(trimethyl)phenyl | 3-(methyl)-7-(propynyl)isocarbostyrilyl |
| 2′methyl, 2′amino, 2′azido, 2′fluro- | 3-(methyl)isocarbostyrilyl |
| cytidine | 4-(fluoro)-6-(methyl)benzimidazole |
| 2′methyl, 2′amino, 2′azido, 2′fluro- | 4-(methyl)benzimidazole |
| adenine | 4-(methyl)indolyl |
| 2′methyl, 2′amino, 2′azido, 2′fluro- | 4,6-(dimethyl)indolyl |
| uridine | 5 nitroindole |
| 2′-amino-2′-deoxyribose | 5 substituted pyrimidines |
| 2-amino-6-Chloro-purine | 5-(methyl)isocarbostyrilyl |
| 2-aza-inosinyl | 5-nitroindole |
| 2′-azido-2′-deoxyribose | 6-(aza)pyrimidine |
| 2′fluoro-2′-deoxyribose | bis-ortho-substituted-6-phenyl-pyrrolo- |
| 6-(azo)thymine | pyrimidin-2-on-3-yl |
| 6-(methyl)-7-(aza)indolyl | difluorotolyl |
| 6-chloro-purine | hypoxanthine |
| 6-phenyl-pyrrolo-pyrimidin-2-on-3-yl | imidizopyridinyl |
| 7-(aminoalkylhydroxy)-1-(aza)-2-(thio)- | inosinyl |
| 3-(aza)-phenthiazin-1-yl | isocarbostyrilyl |
| 7-(aminoalkylhydroxy)-1-(aza)-2-(thio)- | isoguanosine |
| 3-(aza)-phenoxazin-1-yl | N2-substituted purines |
| 7-(aminoalkylhydroxy)-1,3-(diaza)-2- | N6-methyl-2-amino-purine |
| (oxo)-phenoxazin-1-yl | N6-substituted purines |
| 7-(aminoalkylhydroxy)-1,3-(diaza)-2- | N-alkylated derivative |
| (oxo)-phenthiazin-1-yl | napthalenyl |
| 7-(aminoalkylhydroxy)-1,3-(diaza)-2- | nitrobenzimidazolyl |
| (oxo)-phenoxazin-1-yl | nitroimidazolyl |
| 7-(aza)indolyl | nitroindazolyl |
| 7-(guanidiniumalkylhydroxy)-1-(aza)-2- | nitropyrazolyl |
| (thio)-3-(aza)-phenoxazinl-yl | nubularine |
| 7-(guanidiniumalkylhydroxy)-1-(aza)-2- | O6-substituted purines |
| (thio)-3-(aza)-phenthiazin-1-yl | O-alkylated derivative |
| 7-(guanidiniumalkylhydroxy)-1-(aza)-2- | ortho-(aminoalkylhydroxy)-6-phenyl- |
| (thio)-3-(aza)-phenoxazin-1-yl | pyrrolo-pyrimidin-2-on-3-yl |
| 7-(guanidiniumalkylhydroxy)-1,3- | ortho-substituted-6-phenyl-pyrrolo- |
| (diaza)-2-(oxo)-phenoxazin-1-yl | pyrimidin-2-on-3-yl |
| 7-(guanidiniumalkyl-hydroxy)-1,3- | Oxoformycin TP |
| (diaza)-2-(oxo)-phenoxazin-1-yl | para-(aminoalkylhydroxy)-6-phenyl- |
| 7-(guanidiniumalkylhydroxy)-1,3- | pyrrolo-pyrimidin-2-on-3-yl |
| (diaza)-2-(oxo)-phenoxazin-1-yl | para-substituted-6-phenyl-pyrrolo- |
| 7-(propynyl)isocarbostyrilyl | pyrimidin-2-on-3-yl |
| 7-(propynyl)isocarbostyrilyl, propynyl- | pentacenyl |
| 7-(aza)indolyl | phenanthracenyl |
| 7-deaza-inosinyl | phenyl |
| 7-substituted 1-(aza)-2-(thio)-3-(aza)- | propynyl-7-(aza)indolyl |
| phenthiazin-1-yl | pyrenyl |
| 7-substituted 1,3-(diaza)-2-(oxo)- | pyridopyrimidin-3-yl |
| phenoxazin-1-yl | pyridopyrimidin-3-yl, 2-oxo-7-amino- |
| 9-(methyl)-imidizopyridinyl | pyridopyrimidin-3-yl |
| aminoindolyl | pyrrolo-pyrimidin-2-on-3-yl |
| anthracenyl | pyrrolopyrimidinyl |
| bis-ortho-(aminoalkylhydroxy)-6- | pyrrolopyrizinyl |
| phenyl-pyrrolo-nvrimidin-2-on-3-yl | Formycin A |
| stilbenzyl | Formycin B |
| substituted 1,2,4-triazoles | Pyrrolosine |
| tetracenyl | 2′-OH-ara-adenosine |
| tubercidine | 2′-OH-ara-cytidine |
| xanthine | 2′-OH-ara-uridine |
| Xanthosine | 2′-OH-ara-guanosine |
| 2-thio-zebularine | 5-(2-carbomethoxyvinyl)uridine |
| 5-aza-2-thio-zebularine | N6-(19-Amino- |
| 7-deaza-2-amino-purine | pentaoxanonadecyl)adenosine |
| pyridin-4-one ribonucleoside | |
| 2-Amino-riboside | |

In an embodiment, a TREM, a TREM core fragment or a TREM fragment described herein comprises a non-naturally occurring modification provided in Table 8, or a combination thereof.

TABLE 8
Exemplary backbone modifications

| Modification | Modification |
|---|---|
| 3′-alkylene phosphonates | oligonucleosides with heteroatom |
| 3′-amino phosphoramidate | intenucleoside linkage |
| alkene containing backbones | phosphinates |
| aminoalkylphosphotriesters | phosphoramidates |
| aminoalkylphosphoramidates | phosphorothioates |
| boranophosphates | phosphorothioate intenucleoside |
| —CH2-0-N(CH3)—CH2— | linkages |
| —CH2—N(CH3)—N(CH3)—CH2— | phosphorodithioates |
| —CH2—NH—CH2— | phosphotriesters |
| chiral phosphonates | PNA |
| chiral phosphorothioates | siloxane backbones |
| formacetyl and thioformacetyl | sulfamate backbones |
| backbones | sulfide sulfoxide and sulfone |
| methylene (methylimino) | backbones |
| methylene formacetyl and | sulfonate and sulfonamide backbones |
| thioformacetyl backbones | thionoalkylphosphonates |
| methyleneimino and | thionoalkylphosphotriesters |
| methylenehydrazino backbones | thionophosphoramidates |
| morpholino linkages | methylphosphonates |
| —N(CH3)—CH2—CH2— | phosphonoacetates |
| Constrained nucleic acid (CNA) | Phosphorothioate |
| 2′-O-methyl | Constrained nucleic acid (CNA) |
| 2′-O-methoxyethyl (MOE) | 2′-O-methyl |
| 2′ Fluoro | 2′-O-methoxyethyl (MOE) |
| Locked nucleic acid (LNA) | 2′ Fluoro |
| (S)-constrained ethyl (cEt) | Locked nucleic acid (LNA) |
| Fluoro hexitol nucleic acid (FHNA) | (S)-constrained ethyl (cEt) |
| 5′-phosphorothioate | Fluoro hexitol nucleic acid (FHNA) |
| Phosphorodiamidate Morpholino Oligomer | 5′-phosphorothioate |
| (PMO) | Phosphorodiamidate Morpholino Oligomer |
| Tricyclo-DNA (tcDNA) | (PMO) |
| (S) 5′-C-methyl | Tricyclo-DNA (tcDNA) |
| (E)-vinylphosphonate | (S) 5′-C-methyl |
| Methyl phosphonate | (E)-vinylphosphonate |
| (S) 5′-C-methyl with phosphate | Methyl phosphonate |
| (R) 5′-C-methyl with phosphate | (S) 5′-C-methyl with phosphate |
| DNA | (R) 5′-C-methyl with phosphate |
| (R) 5′-C-methyl | DNA |
| GNA (glycol nucleic acid) | (R) 5′-C-methyl |
| alkyl phosphonates | GNA (glycol nucleic acid) |
| Phosphorothioate | alkyl phosphonates |

In an embodiment, a TREM, a TREM core fragment or a TREM fragment described herein comprises a non-naturally occurring modification provided in Table 9, or a combination thereof.

TABLE 9
Exemplary non-naturally occurring backbone modificiations
Name of synthetic backbone modifications

Phosphorothioate
Constrained nucleic acid (CNA)
2′ O′methylation
2′-O-methoxyethylribose (MOE)
2′ Fluoro
Locked nucleic acid (LNA)
(S)-constrained ethyl (cEt)
Fluoro hexitol nucleic acid (FHNA)
5′phosphorothioate
Phosphorodiamidate Morpholino Oligomer (PMO)
Tricyclo-DNA (tcDNA)
(S) 5′-C-methyl
(E)-vinylphosphonate
Methyl phosphonate
(S) 5′-C-methyl with phosphate

### Design Guidance

The present disclosure further describes representative design principles for installing a non-naturally occurring modification on a TREM, TREM core fragment, or TREM fragment. Without being bound by theory, these design principles may provide guidance for modulating a parameter of a TREM, TREM core fragment, or TREM fragment described herein. These design principles are also referred to herein as “Design Guidances”. For example, a TREM comprising a non-naturally occurring modification pattern according to a Design Guidance may exhibit improved stability, e.g., in vitro or in a cell. Representative Design Guidances are described in greater detail below.

### Design Guidance 1

In an embodiment, a TREM comprises a sequence of Formula (I): [L1]-[ASt Domain1]-[L2]-[DH Domain]-[L3]-[ACH Domain]-[VL Domain]-[TH Domain]-[L4]-[ASt Domain2], wherein independently, [L1] and [VL Domain], are optional; and one of [L1], [ASt Domain1], [L4], and [ASt Domain2] comprises a nucleotide having a non-naturally occurring modification.

(a) In an embodiment, the nucleotide having a non-naturally occurring modification is any one of nucleotide positions 1-6 or 66-76., e.g., (i) wherein the nucleotide position corresponds to 1-6 or 66-76 of SEQ ID NO: 622 or (ii) wherein the nucleotide position corresponds to 1-6 or 66-76 according to the CtNS.

(b) In an embodiment, one of [L1], [ASt Domain1], [L4], and [ASt Domain2] comprises a nucleotide having a 2′-O-methoxy (2′O-Me) modification. In an embodiment, the nucleotide having the 2′OMe modification is any one of nucleotide positions 1-6, 65-70, and 74-76, e.g., (i) wherein the nucleotide position corresponds to 1-6, 65-70, and 74-76 of SEQ ID NO: 622 or (ii) wherein the nucleotide position corresponds to 1-6, 65-70, and 74-76 according to the CtNS.

(c) In an embodiment, one of [L1], [ASt Domain1], [L4], and [ASt Domain2] comprises a nucleotide having a phosphorothiorate (PS) modification. In an embodiment, the PS modiciation is present on any one of nucleotide positions 1-6 or 65-76, e.g., (i) wherein the nucleotide position corresponds to 1-6 or 65-76 of SEQ ID NO: 622 or (ii) wherein the nucleotide position corresponds to 1-6 or 65-76 according to the CtNS.

(d) In an embodiment, one of [L1], [ASt Domain1], [L4], and [ASt Domain2] comprises a nucleotide having a 2′-O-methoxy (2′O-Me) modification and a nucleotide having a phosphorothiorate (PS) modification. In an embodiment, both the 2′-OMe and the PS modiciation are independently present on any one of nucleotide positions 1-6 or 65-76, e.g., (i) wherein the nucleotide position corresponds to 1-6 or 65-76 of SEQ ID NO: 622 or (ii) wherein the nucleotide position corresponds to 1-6 or 65-76 according to the CtNS.

(e) In an embodiment, one of [L1]. [ASt Domain1]. [L4], and [ASt Domain2] comprises a nucleotide having a 2′-O-methoxy (2′O-Me) modification and a nucleotide having a phosphorothiorate (PS) modification, provided that none of nucleotides corresponding to positions 71-73 (e.g., corresponding to SEQ ID NO: 622 or the CtNS) comprise a 2′-O-methoxy (2′O-Me) modification.

(f) In an embodiment, one of [L1], [ASt Domain1], [L4], and [ASt Domain2] comprises a nucleotide having a phosphorothiorate (PS) modification on one of positions 1-3 or 74-76 (e.g., corresponding to SEQ ID NO: 622 or the CtNS).

### Design Guidance 2

In an embodiment, a TREM comprises a sequence of Formula (I): [L1]-[ASt Domain1]-[L2]-[DH Domain]-[L3]-[ACH Domain]-[VL Domain]-[TH Domain]-[L4]-[ASt Domain2], wherein independently. [L1] and [VL Domain], are optional; and one of [DH Domain]-[L3] comprises a nucleotide having a non-naturally occurring modification.

(a) In an embodiment, the nucleotide having a non-naturally occurring modification is any one of nucleotide positions 10-13 or 22-25., e.g., (i) wherein the nucleotide position corresponds to 10-13 or 22-25 of SEQ ID NO: 622 or (ii) wherein the nucleotide position corresponds to 10-13 or 22-25 according to the CtNS.

(b) In an embodiment, one of [DH Domain]-[L3] comprises a nucleotide having a 2′-O-methoxy (2′O-Me) modification. In an embodiment, the nucleotide having the 2′OMe modification is any one of nucleotide positions 10-13 or 22-25., e.g., (i) wherein the nucleotide position corresponds to 10-13 or 22-25 of SEQ ID NO: 622 or (ii) wherein the nucleotide position corresponds to 10-13 or 22-25 according to the CtNS.

(c) In an embodiment, one of [DH Domain]-[L3] comprises a nucleotide that does not have a phosphorothioate (PS) modification. In an embodiment, the nucleotide that does not comprise the PS modification is any one of nucleotide positions 10-13 or 22-25., e.g., (i) wherein the nucleotide position corresponds to 10-13 or 22-25 of SEQ ID NO: 622 or (ii) wherein the nucleotide position corresponds to 10-13 or 22-25 according to the CtNS.

### Design Guidance 3

In an embodiment, a TREM comprises a sequence of Formula (I): [L1]-[ASt Domain1]-[L2]-[DH Domain]-[L3]-[ACH Domain]-[VL Domain]-[TH Domain]-[L4]-[ASt Domain2], wherein independently, [L1] and [VL Domain], are optional; and the [ACH Domain] does not comprise a 2′-nucleotide sugar modification (e.g., 2′-ribose modification).

(a) In an embodiment, the [ACH Domain] comprises a nucleotide having an internucleotide modification. In an embodiment, the nucleotide having the internucleotide modification is present on any one of nucleotide positions 32-28, e.g., (i) wherein the nucleotide position corresponds to any one of 32-38 of SEQ ID NO: 622 or (ii) wherein the nucleotide position corresponds to any one of 32-38 according to the CtNS.

(b) In an embodiment, the [ACH Domain] comprises a nucleotide having a phosphorothioate (PS) modification. In an embodiment, the nucleotide having the PS modification is present on any one of nucleotide positions 32-28, e.g., (i) wherein the nucleotide position corresponds to any one of 32-38 of SEQ ID NO: 622 or (ii) wherein the nucleotide position corresponds to any one of 32-38 according to the CtNS.

(c) In an embodiment, the [ACH Domain] comprises a nucleotide that does not have a 2′-nucleotide sugar modification. In an embodiment, the [ACH Domain] comprises a nucleotide that does not have a 2′-O-methoxy (2′O-Me) modification.

(d) In an embodiment, the [ACH Domain] comprises a nucleotide having an internucleotide modification (e.g., a PS modification) and a nucleotide that does not have a 2′-nucleotide sugar modification (e.g., a 2′O-Me modification).

(e) In an embodiment, the [ACH Domain] comprises a nucleotide having a 2′-fluoro (2′F) modification. In an embodiment, the nucleotide having the 2′F modification is nucleotide position 33, e.g., (i) wherein the nucleotide position corresponds to 33 of SEQ ID NO: 622 or (ii) wherein the nucleotide position corresponds to 33 according to the CtNS.

(f) In an embodiment, the [ACH Domain] comprises a nucleotide having an (i) internucleotide modification (e.g., a PS modification); (ii) a nucleotide having a 2′F modification; and (iii) a nucleotide that does not have a 2′OMe modification. In an embodiment, the nucleotide having the 2′F modification is nucleotide position 33, e.g., (i) wherein the nucleotide position corresponds to 33 of SEQ ID NO: 622 or (ii) wherein the nucleotide position corresponds to 33 according to the CtNS.

(g) In an embodiment, the [ACH Domain] comprises a nucleotide having a phosphorothioate (PS) modification on the anticodon, e.g., at any one of positions 34-36, (i) wherein the nucleotide position corresponds to any one of 34-36 of SEQ ID NO: 622 or (ii) wherein the nucleotide position corresponds to any one of 34-36 according to the CtNS.

### Design Guidance 4

In an embodiment, a TREM comprises a sequence of Formula (I): [L1]-[ASt Domain1]-[L2]-[DH Domain]-[L3]-[ACH Domain]-[VL Domain]-[TH Domain]-[L4]-[ASt Domain2], wherein independently, [L1] and [VL Domain], are optional; and the [VL Domain] comprises a nucleotide having a non-naturally occurring modification.

(a) In an embodiment, the [VL Domain] comprises a nucleotide having a 2′-nucleotide sugar modification., e.g., (i) wherein the nucleotide having the 2′-nucleotide sugar modification corresponds to any one of 44-48 of SEQ ID NO: 622 or (ii) wherein the nucleotide having the 2′-nucleotide sugar modification corresponds to any one of V1-V27 and 46-48 according to the CtNS.

(b) In an embodiment, the [VL Domain] comprises a nucleotide having a 2′-OMe modification. In an embodiment, the 2′OMe modification is present on any one of nucleotide positions within the [VL Domain]., e.g., (i) wherein the nucleotide position corresponds to any one of 44-48 of SEQ ID NO: 622 or (ii) wherein the nucleotide position corresponds to any one of V1-V27 and 46-48 according to the CtNS.

(b) In an embodiment, the [VL Domain] comprises a nucleotide having a 2′-F modification. In an embodiment, the F modification is present on any one of nucleotide positions within the [VL Domain]., e.g., (i) wherein the nucleotide position corresponds to any one of 44-48 of SEQ ID NO: 622 or (ii) wherein the nucleotide position corresponds to any one of V1-V27 and 46-48 according to the CtNS.

(d) In an embodiment, the [VL Domain] comprises a nucleotide that does not have an internucleotide modification. In an embodiment, the [VL Domain] comprises a nucleotide that does not have a PS modification, e.g., (i) wherein the nucleotide position corresponds to any one of 44-48 of SEQ ID NO: 622 or (ii) wherein the nucleotide position corresponds to any one of V1-V27 and 46-48 according to the CtNS.

(e) In an embodiment, the [VL Domain] comprises (i) a nucleotide having a 2′-OMe modification; (ii) a nucleotide having a 2′-F modification; and (iii) a nucleotide that does not have an internucleotide modification (e.g., a PS modification). In an embodiment, the nucleotide having the 2′OMe modification, the nucleotide having the 2′F modification, and the nucleotide not having the internucleotide modification (e.g., the PS modification) are present on any one of nucleotide positions within the [VL Domain]., e.g., (i) wherein the nucleotide positions correspond to any one of 44-48 of SEQ ID NO: 622 or (ii) wherein the nucleotide positions correspond to any one of V1-V27 and 46-48 according to the CtNS.

### Design Guidance 5

In an embodiment, a TREM comprises a sequence of Formula (I): [L1]-[ASt Domain1]-[L2]-[DH Domain]-[L3]-[ACH Domain]-[VL Domain]-[TH Domain]-[L4]-[ASt Domain2], wherein independently. [L1] and [VL Domain], are optional; and one of [TH Domain] comprises a nucleotide having a non-naturally occurring modification. In an embodiment, the nucleotide having a non-naturally occurring modification is present on any one of nucleotide positions 49-65., e.g., (i) wherein the nucleotide position corresponds to 49-65 of SEQ ID NO: 622 or (ii) wherein the nucleotide position corresponds to 49-65 according to the CtNS. In an embodiment, the [TH Domain] comprises a nucleotide having a 2′-nucleotide sugar modification. In an embodiment, the nucleotide having the 2′-nucleotide sugar modification is any one of nucleotide positions 49-65, e.g., (i) wherein the nucleotide position corresponds to 49-65 of SEQ ID NO: 622 or (ii) wherein the nucleotide position corresponds to 49-65 according to the CtNS.

(a) In an embodiment, the nucleotide having a non-naturally occurring modification is any one of nucleotide positions 49-53 and 61-65, e.g., (i) wherein the nucleotide position corresponds to 49-53 and 61-65 of SEQ ID NO: 622 or (ii) wherein the nucleotide position corresponds to 49-53 and 61-65 according to the CtNS.

(b) In an embodiment, the [TH Domain] comprises a nucleotide having a 2′-OMe modification. In an embodiment, the nucleotide having the 2′-OMe modification is any one of nucleotide positions 49-53, 61-62, and 64-65, e.g., (i) wherein the nucleotide position corresponds to 49-53, 61-62, and 64-65 of SEQ ID NO: 622 or (ii) wherein the nucleotide position corresponds to 49-53, 61-62, and 64-65 according to the CtNS.

(c) In an embodiment, the [TH Domain] comprises a nucleotide having a 2′-F modification. In an embodiment, the nucleotide having the 2′-F modification is nucleotide position 63, e.g., (i) wherein the nucleotide position corresponds to 63 of SEQ ID NO: 622 or (ii) wherein the nucleotide position corresponds to 63 according to the CtNS.

(d) In an embodiment, the [TH Domain] comprises a nucleotide that does not have an internucleotide modification (e.g., a PS modification). In an embodiment, the nucleotide not having an internucleotide modification (e.g., a PS modification) is any one of nucleotide positions 49-53 and 61-65, e.g., (i) wherein the nucleotide position corresponds to 49-53 and 61-65 of SEQ ID NO: 622 or (ii) wherein the nucleotide position corresponds to 49-53 and 61-65 according to the CtNS.

### Design Guidance 6

In an embodiment, a TREM comprises a sequence of Formula (I): [L1]-[ASt Domain1]-[L2]-[DH Domain]-[L3]-[ACH Domain]-[VL Domain]-[TH Domain]-[L4]-[ASt Domain2], wherein independently, [L1] and [VL Domain], are optional; and each of [DH Domain] and the [TH Domain] comprises a nucleotide having a non-naturally occurring modification.

(a) In an embodiment, the [DH Domain] comprises a nucleotide having a non-naturally occurring modification at any one of nucleotide positions 14-21 (e.g., 14-20, e.g., 16-18), e.g., (i) wherein the nucleotide position corresponds to 14-21 (e.g., 14-20, e.g., 16-18) of SEQ ID NO: 622 or (ii) wherein the nucleotide position corresponds to 14-21 (e.g., 14-20, e.g., 16-18) according to the CtNS. In an embodiment, the non-naturally occurring modification is a 2′-nucleotide sugar modification (e.g., 2′-OMe).

(b) In an embodiment, the [TH Domain] comprises a nucleotide having a non-naturally occurring modification at any one of nucleotide positions 54-60, e.g., (i) wherein the nucleotide position corresponds to 54-60 of SEQ ID NO: 622 or (ii) wherein the nucleotide position corresponds to 54-60 according to the CtNS. In an embodiment, the non-naturally occurring modification is a 2′-nucleotide sugar modification (e.g., 2′-F).

(c) In an embodiment, the [TH Domain] comprises a nucleotide having a 2′OMe at any one of nucleotide positions 54-60 (e.g., 54, 56, 57, or 59), e.g., (i) wherein the nucleotide position corresponds to 54-60 (e.g., 54, 56, 57, or 59), of SEQ ID NO: 622 or (ii) wherein the nucleotide position corresponds to 54-60 (e.g., 54, 56, 57, or 59), according to the CtNS.

(d) In an embodiment, the [TH Domain] comprises a nucleotide having a 2′-F at any one of nucleotide positions 54-60 (e.g., 57, 58, or 60), e.g., (i) wherein the nucleotide position corresponds to 54-60 (e.g., 57, 58, or 60) of SEQ ID NO: 622 or (ii) wherein the nucleotide position corresponds to 54-60 (e.g., 57, 58, or 60) according to the CtNS.

(e) In an embodiment, the TREM comprises a non-naturally occurring modification in both of the [DH Domain] and the [TH Domain], wherein (i) the [DH Domain] comprises a nucleotide having a non-naturally occurring modification at any one of nucleotide positions 14-21 (e.g., 14-20, e.g., 16-18), e.g., (i) wherein the nucleotide position corresponds to 14-21 (e.g., 14-20, e.g., 16-18) of SEQ ID NO: 622 or (ii) wherein the nucleotide position corresponds to 14-21 (e.g., 14-20, e.g., 16-18) according to the CtNS (ii) the [TH Domain] comprises a nucleotide having a non-naturally occurring modification at any one of nucleotide positions 54-60, e.g., (i) wherein the nucleotide position corresponds to 54-60 of SEQ ID NO: 622 or (ii) wherein the nucleotide position corresponds to 54-60 according to the CtNS. In some embodiments, the non-naturally occurring modification is selected from a 2′OMe or 2′F modification.

Table 21 below summarizes a set of representative TREMs described herein correlated to the Design Guidances outlined above, e.g., Design Guidances 1-6.

TABLE 21
Exemplary TREMs described herein correlated
to Design Guidances described herein

| TREM | SEQ ID | Design Guidance | | | | | |
|---|---|---|---|---|---|---|---|
| NO. | NO. | 1 | 2 | 3 | 4 | 5 | 6 |
| 1 | 622 | | | | | | |
| 2 | 623 | | | | | | |
| 3 | 624 | | | | | | |
| 5 | 626 | | c | c | d | | |
| 6 | 627 | | c | c | d | | |
| 7 | 629 | | c | c | d | | |
| 8 | 630 | | c | c | d | | |
| 10 | 631 | | c | c | d | | |
| 11 | 632 | | c | c | d | | |
| 12 | 633 | | c | c | d | | a |
| 13 | 634 | | c | c | d | | |
| 14 | 635 | | c | c | d | | b |
| 15 | 636 | abe | abc | c | d | | |
| 16 | 637 | abe | c | c | d | | a |
| 17 | 638 | abe | c | c | d | | a |
| 18 | 639 | abe | c | c | d | | a |
| 19 | 640 | abe | c | c | d | | |
| 20 | 641 | abe | c | c | abde | ab | |
| 21 | 642 | abe | c | c | d | ab | |
| 22 | 643 | abe | c | c | d | ab | |
| 23 | 644 | abe | c | c | d | ab | |
| 24 | 645 | abe | c | c | d | | |
| 25 | 646 | | abc | c | d | | a |
| 26 | 647 | | abc | c | d | | a |
| 27 | 648 | | abc | c | d | | a |
| 28 | 649 | | abc | c | d | | |
| 29 | 650 | abe | c | c | d | ab | |
| 30 | 651 | | abc | c | abde | ab | |
| 31 | 652 | | abc | c | d | ab | |
| 32 | 653 | | abc | c | d | ab | |
| 33 | 654 | | abc | c | d | ab | |
| 34 | 655 | | abc | c | d | | |
| 35 | 656 | | c | c | d | | a |
| 36 | 657 | | c | c | d | | a |
| 37 | 658 | | c | c | d | | a |
| 38 | 659 | | c | c | abde | ab | a |
| 39 | 660 | | c | c | d | ab | a |
| 40 | 661 | | c | c | d | ab | a |
| 41 | 662 | | c | c | d | ab | a |
| 42 | 663 | | c | c | d | | a |
| 43 | 664 | | c | c | d | | a |
| 44 | 665 | | c | c | d | | a |
| 45 | 666 | | c | c | abde | ab | a |
| 46 | 667 | | c | c | d | ab | a |
| 47 | 668 | abe | c | c | d | ab | |
| 48 | 669 | | c | c | d | ab | a |
| 49 | 670 | | c | c | d | ab | a |
| 50 | 671 | | c | c | d | | a |
| 51 | 672 | | c | c | d | | a |
| 52 | 673 | | c | c | abde | ab | a |
| 53 | 674 | | c | c | d | ab | a |
| 54 | 675 | | c | c | d | ab | a |
| 55 | 676 | | c | c | d | ab | a |
| 56 | 677 | | c | c | d | | a |
| 57 | 678 | | c | c | abde | ab | |
| 58 | 679 | | c | c | d | ab | |
| 59 | 680 | | c | c | d | ab | |
| 60 | 681 | | c | c | d | ab | |
| 61 | 682 | abe | c | c | d | ab | a |
| 63 | 684 | | c | c | d | | |
| 64 | 685 | | c | c | abde | ab | |
| 65 | 686 | | c | c | abde | ab | |
| 66 | 687 | | c | c | abde | ab | |
| 67 | 688 | | c | c | abde | ab | |
| 68 | 689 | | c | c | d | ab | |
| 69 | 690 | | c | c | d | ab | |
| 70 | 691 | | c | c | d | ab | |
| 71 | 692 | | c | c | d | ab | |
| 72 | 693 | | c | c | d | ab | |
| 73 | 694 | | c | c | d | ab | |
| 74 | 695 | | c | c | d | | a |
| 75 | 696 | | c | c | d | | a |
| 76 | 697 | | abc | c | d | | |
| 77 | 698 | | c | c | d | ab | |
| 78 | 699 | abe | c | c | d | ab | a |
| 81 | 702 | | c | c | d | | a |
| 82 | 703 | | c | c | d | | a |
| 83 | 704 | | abc | c | d | | |
| 84 | 705 | | c | c | d | ab | |
| 85 | 706 | | c | c | d | | abce |
| 86 | 707 | | c | c | d | | abce |
| 87 | 708 | | abc | c | d | | bc |
| 88 | 709 | | c | c | d | ab | bc |
| 89 | 710 | abe | c | c | d | | |
| 90 | 711 | abe | c | c | d | | |
| 91 | 712 | abe | c | c | d | | |
| 92 | 713 | | c | c | d | | |
| 93 | 714 | | c | c | d | ab | |
| 94 | 715 | | c | c | d | ab | |
| 95 | 716 | | c | c | d | ab | |
| 96 | 717 | | c | c | d | ab | |

### TREM, TREM Core Fragment and TREM Fragment Fusions

In an embodiment, a TREM, a TREM core fragment or a TREM fragment disclosed herein comprises an additional moiety, e.g., a fusion moiety. In an embodiment, the fusion moiety can be used for purification, to alter folding of the TREM, TREM core fragment or TREM fragment, or as a targeting moiety. In an embodiment, the fusion moiety can comprise a tag, a linker, can be cleavable or can include a binding site for an enzyme. In an embodiment, the fusion moiety can be disposed at the N terminal of the TREM or at the C terminal of the TREM, TREM core fragment or TREM fragment. In an embodiment, the fusion moiety can be encoded by the same or different nucleic acid molecule that encodes the TREM, TREM core fragment or TREM fragment.

### TREM Consensus Sequence

In an embodiment, a TREM disclosed herein comprises a consensus sequence provided herein.

In an embodiment, a TREM disclosed herein comprises a consensus sequence of Formula I zzz, wherein zzz indicates any of the twenty amino acids and Formula I corresponds to all species.

In an embodiment, a TREM disclosed herein comprises a consensus sequence of Formula

II zzz, wherein zzz indicates any of the twenty amino acids and Formula II corresponds to mammals.

In an embodiment, a TREM disclosed herein comprises a consensus sequence of Formula III zzz, wherein zzz indicates any of the twenty amino acids and Formula III corresponds to humans.

In an embodiment, zzz indicates any of the twenty amino acids: alanine, arginine, asparagine, aspartate, cysteine, glutamine, glutamate, glycine, histidine, isoleucine, methionine, leucine, lysine, phenylalanine, proline, serine, threonine, tryptophan, tyrosine, or valine.

In an embodiment, a TREM disclosed herein comprises a property selected from the following:
a) under physiological conditions residue R₀ forms a linker region, e.g., a Linker 1 region;
b) under physiological conditions residues R₁-R₂-R₃-R₄-R₅-R₆-R₇ and residues R₆₅-R₆₆-R₆₇-R₆₈-R₆₉-R₇₀-R₇₁ form a stem region, e.g., an AStD stem region;
c) under physiological conditions residues R₈-R₉ forms a linker region, e.g., a Linker 2 region;
d) under physiological conditions residues-R₁₀-R₁₁-R₁₂-R₁₃-R₁₄ R₁₅-R₁₆-R₁₇-R₁₈-R₁₉-R₂₀-R₂₁-R₂₂-R₂₃-R₂₄-R₂₅-R₂₆-R₂₇-R₂₈ form a stem-loop region, e.g., a D arm Region;
e) under physiological conditions residue-R₂₉ forms a linker region, e.g., a Linker 3 Region;
f) under physiological conditions residues-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ form a stem-loop region, e.g., an AC arm region;
g) under physiological conditions residue-[R₄₇]ₓ comprises a variable region, e.g., as described herein;
h) under physiological conditions residues-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ form a stem-loop region, e.g., a T arm Region; or
i) under physiological conditions residue R₇₂ forms a linker region, e.g., a Linker 4 region.

### Alanine TREM Consensus Sequence

In an embodiment, a TREM disclosed herein comprises the sequence of Formula I_{ALA} (SEQ ID NO: 562),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂, wherein R is a ribonucleotide residue and the consensus for Ala is: R₀=absent; R₁₄, R₅₇=are independently A or absent; R₂₆=A, C, G or absent; R₅, R₆, R₁₅, R₁₆, R₂₁, R₃₀, R₃₁, R₃₂, R₃₄, R₃₇, R₄₁, R₄₂, R₄₃, R₄₄, R₄₅, R₄₈, R₄₉, R₅₀, R₅₈, R₅₉, R₆₃, R₆₄, R₆₆, R₆₇=are independently N or absent; R₁, R₃₅, R₆₅=are independently A, C, U or absent; R₁, R₉, R₂₀, R₃₈, R₄₀, R₅₁, R₅₂, R₅₆=are independently A, G or absent; R₇, R₂₂, R₂₅, R₂₇, R₂₉, R₄₆, R₅₃, R₇₂=are independently A, G, U or absent; R₂₄, R₆₉=are independently A, U or absent; R₇₀, R₇₁=are independently C or absent; R₃, R₄=are independently C, G or absent; R₁₂, R₃₃, R₃₆, R₆₂, R₆₈=are independently C, G, U or absent; R₁₃, R₁₇, R₂₈, R₃₉, R₅₅, R₀, R₆₁=are independently C, U or absent; R₁₀, R₁₉, R₂₃=are independently G or absent; R₂=G, U or absent; R₈, R₁₈, R₅₄=are independently U or absent; [R₄₇]ₓ=N or absent; wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{ALA} (SEQ ID NO: 563),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ala is:
R₀, R₁₈=are absent;
R₁₄, R₂₄, R₅₇=are independently A or absent;
R₁₅, R₂₆, R₆₄=are independently A, C, G or absent;
R₁₆, R₃₁, R₅₀, R₅₉=are independently N or absent;
R₁₁, R₃₂, R₃₇, R₄₁, R₄₃, R₄₅, R₄₉, R₆₅, R₆₆=are independently A, C, U or absent;
R₁, R₅, R₉, R₂₅, R₂₇, R₃₈, R₄₀, R₄₆, R₅₁, R₅₆=are independently A, G or absent;
R₇, R₂₂, R₂₉, R₄₂, R₄₄, R₅₃, R₆₃, R₇₂=are independently A, G, U or absent;
R₆, R₃₅, R₆₉=are independently A, U or absent;
R₅₅, R₆₀, R₀, R₇₁=are independently C or absent;
R₃=C, G or absent;
R₁₂, R₃₆, R₄₈=are independently C, G, U or absent;
R₁₃, R₁₇, R₂₈, R₃₀, R₃₄, R₃₉, R₅₅, R₆₁, R₆₂, R₆₇, R₆₈=are independently C, U or absent;
R₄, R₁₀, R₁₉, R₂₀, R₂₃, R₅₂=are independently G or absent;
R₂, R₈, R₃₃=are independently G, U or absent;
R₂₁, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{ALA} (SEQ ID NO: 564),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ala is:
R₀, R₁₈=are absent;
R₁₄, R₂₄, R₅₇, R₇₂=are independently A or absent;
R₁₅, R₂₆, R₆₄=are independently A, C, G or absent;
R₁₆, R₃₁, R₅₀=are independently N or absent;
R₁₁, R₃₂, R₃₇, R₄₁, R₄₃, R₄₅, R₄₉, R₆₅, R₆₆=are independently A, C, U or absent;
R₅, R₉, R₂₅, R₂₇, R₃₈, R₄₀, R₄₆, R₅₁, R₅₆=are independently A, G or absent;
R₇, R₂₂, R₂₉, R₄₂, R₄₄, R₅₃, R₆₃=are independently A, G, U or absent;
R₆, R₃₅=are independently A, U or absent;
R₅₅, R₆₀, R₆₁, R₇₀, R₇₁=are independently C or absent;
R₁₂, R₄₈, R₅₉=are independently C, G, U or absent;
R₁₃, R₁₇, R₂₈, R₃₀, R₃₄, R₃₉, R₅₈, R₆₂, R₆₇, R₆₈=are independently C, U or absent;
R₁, R₂, R₃, R₄, R₁₀, R₁₉, R₂₀, R₂₃, R₅₂=are independently G or absent;
R₃₃, R₃₆=are independently G, U or absent;
R₈, R₂₁, R₅₄, R₆₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Arginine TREM Consensus Sequence

In an embodiment, a TREM disclosed herein comprises the sequence of Formula I ARG (SEQ ID NO: 565),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
   wherein R is a ribonucleotide residue and the consensus for Arg is:

R₅₇=A or absent;

R₉, R₂₇=are independently A, C, G or absent;
R₁, R₂, R₃, R₄, R₅, R₆, R₇, R₁₁, R₁₂, R₁₆, R₂₁, R₂₂, R₂₃, R₂₅, R₂₆, R₂₉, R₃₀, R₃₁, R₃₂, R₃₃, R₃₄, R₃₇, R₄₂, R₄₄, R₄₅,
R₄₆, R₄₈, R₄₉, R₅₀, R₅₁, R₅₈, R₆₂, R₆₃, R₆₄, R₆₅, R₆₆, R₆₇, R₆₈, R₆₉, R₇₀, R₇₁=are independently N or
absent;
R₁₃, R₁₇, R₄₁=are independently A, C, U or absent;
R₁₉, R₂₀, R₂₄, R₄₀, R₅₆=are independently A, G or absent;
R₁₄, R₁₅, R₇₂=are independently A, G, U or absent;
R₁₈=A, U or absent;
R₃₈=C or absent;
R₃₅, R₄₃, R₆₁=are independently C, G, U or absent;
R₂₈, R₅₅, R₅₉, R₆₀=are independently C, U or absent;
R₀, R₁₀, R₅₂=are independently G or absent;
R₈, R₃₉=are independently G, U or absent;
R₃₆, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II ARG (SEQ ID NO: 566),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Arg is:
R₁₈=absent;
R₂₄, R₅₇=are independently A or absent;
R₄₁=A, C or absent;
R₃, R₇, R₃₄, R₅₀=are independently A, C, G or absent;
R₂, R₅, R₆, R₁₂, R₂₆, R₃₂, R₃₇, R₄₄, R₅₈, R₆₆, R₆₇, R₆₈, R₇₀=are independently N or absent;
R₄₉, R₇₁=are independently A, C, U or absent;
R₁, R₁₅, R₁₉, R₂₅, R₂₇, R₄₀, R₄₅, R₄₆, R₅₆, R₇₂=are independently A, G or absent;
R₁₄, R₂₉, R₆₃=are independently A, G, U or absent;
R₁₆, R₂₁=are independently A, U or absent;
R₃₈, R₆₁=are independently C or absent;
R₃₃, R₄₈=are independently C, G or absent;
R₄, R₉, R₁₁, R₄₃, R₆₂, R₆₄, R₆₉=are independently C, G, U or absent;
R₁₃, R₂₂, R₂₈, R₃₀, R₃₁, R₃₅, R₅₅, R₆₀, R₆₅=are independently C, U or absent;
R₀, R₁₀, R₂₀, R₂₃, R₅₁, R₅₂=are independently G or absent;
R₈, R₃₉, R₄₂=are independently G, U or absent;
R₁₇, R₃₆, R₅₃, R₅₄, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III ARG (SEQ ID NO: 567),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Arg is:
R₁₈=is absent;
R₁₅, R₂₁, R₂₄, R₄₁, R₅₇=are independently A or absent;
R₃₄, R₄₄=are independently A, C or absent;
R₃, R₅, R₅₅=are independently A, C, G or absent;
R₂, R₆, R₆₆, R₇₀=are independently N or absent;
R₃₇, R₄₉=are independently A, C, U or absent;
R₁, R₂₅, R₂₉, R₄₀, R₄₅, R₄₆, R₅₀=are independently A, G or absent;
R₁₄, R₆₃, R₆₈=are independently A, G, U or absent;
R₁₆=A, U or absent;
R₃₈, R₆₁=are independently C or absent;
R₇, R₁₁, R₁₂, R₂₆, R₄₈=are independently C, G or absent;
R₆₄, R₆₇, R₆₉=are independently C, G, U or absent;
R₄, R₁₃, R₂₂, R₂₈, R₃₀, R₃₁, R₃₅, R₄₃, R₅₅, R₆₀, R₆₂, R₆₅, R₇₁=are independently C, U or absent;
R₀, R₁₀, R₁₉, R₂₀, R₂₃, R₂₇, R₃₃, R₅₁, R$2, R₅₆, R₇₂=are independently G or absent;
R₈, R₉, R₃₂, R₃₉, R₄₂=are independently G, U or absent;
R₁₇, R₃₆, R₅₃, R₅₄, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Asparagine TREM Consensus Sequence

In an embodiment, a TREM disclosed herein comprises the sequence of Formula I ASN (SEQ ID NO: 568),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Asn is:
R₀, R₁₈=are absent;
R₄₁=A or absent;
R₁₄, R₄₈, R₅₆=are independently A, C, G or absent;
R₂, R₄, R₅, R₆, R₁₂, R₁₇, R₂₆, R₂₉, R₃₀, R₃₁, R₄₄, R₄₅, R₄₆, R₄₉, R so, R₅₈, R₆₂, R₆₃, R₆₅, R₆₆, R₆₇, R₆₈, R₇₀, R₇₁=are independently N or absent;
R₁, R₁₃, R₂₂, R₄₂, R₅₅, R₅₉=are independently A, C, U or absent;
R₉, R₁₅, R₂₄, R₂₇, R₃₄, R₃₇, R₅₁, R₇₂=are independently A, G or absent;
R₁, R₇, R₂₅, R₆₉=are independently A, G, U or absent;
R₄₀, R₅₇=are independently A, U or absent;
R₆₀=C or absent;
R₃₃=C, G or absent;
R₂₁, R₃₂, R₄₃, R₆₄=are independently C, G, U or absent;
R₃, R₁₆, R₂₈, R₃₅, R₃₆, R₆₁=are independently C, U or absent;
R₁₀, R₁₉, R₂₀, R₅₂=are independently G or absent;
R₅₄=G, U or absent;
R₈, R₂₃, R₃₈, R₃₉, R₅₃=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II ASN (SEQ ID NO: 569),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Asn is:
R₀, R₁₈=are absent
R₂₄, R₄₁, R₄₆, R₆₂=are independently A or absent;
R₅₉=A, C or absent;
R₁₄, R₅₆, R₆₆=are independently A, C, G or absent;
R₁₇, R₂₉=are independently N or absent;
R₁₁, R₂₆, R₄₂, R₅₅=are independently A, C, U or absent;
R₁, R₉, R₁₂, R₁₅, R₂₅, R₃₄, R₃₇, R₄₈, R$1, R₆₇, R₆₈, R₆₉, R₇₀, R₇₂=are independently A, G or absent;
R₄₄, R₄₅, R₅₅=are independently A, G, U or absent;
R₄₀, R₅₇=are independently A, U or absent;
R₅, R₂₈, R₆₀=are independently C or absent;
R₃₃, R₆₅=are independently C, G or absent;
R₂₁, R₄₃, R₇₁=are independently C, G, U or absent;
R₃, R₆, R₁₃, R₂₂, R₃₂, R₃₅, R₃₆, R₆₁, R₆₃, R₆₄=are independently C, U or absent;
R₇, R₁₀, R₁₉, R₂₀, R₂₇, R₄₉, R₅₂=are independently G or absent;
R₅₄=G, U or absent;
R₂, R₄, R₈, R₁₆, R₂₃, R₃₀, R₃₁, R₃₈, R₃₉, R₅₀, R₅₃=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III ASN (SEQ ID NO: 570),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Asn is:
R₀, R₁₈=are absent
R₂₄, R₄₀, R₄₁, R₄₆, R₆₂=are independently A or absent;
R₅₉=A, C or absent;
R₁₄, R₅₆, R₆₆=are independently A, C, G or absent;
R₁₁, R₂₆, R₄₂, R₅₅=are independently A, C, U or absent;
R₁, R₉, R₁₂, R₁₅, R₃₄, R₃₇, R₄₈, R₅₁, R₆₇, R₆₈, R₆₉, R₇₀=are independently A, G or absent;
R₄₄, R₄₅, R₅₅=are independently A, G, U or absent;
R₅₇=A, U or absent;
R₅, R₂₈, R₆₀=are independently C or absent;
R₃₃, R₆₅=are independently C, G or absent;
R₁₇, R₂₁, R₂₉=are independently C, G, U or absent;
R₃, R₆, R₁₃, R₂₂, R₃₂, R₃₅, R₃₆, R₄₃, R₆₁, R₆₃, R₆₄, R₇₁=are independently C, U or absent;
R₇, R₁₀, R₁₉, R₂₀, R₂₅, R₂₇, R₄₉, R $2, R₇₂=are independently G or absent;
R₅₄=G, U or absent;
R₂, R₄, R&, R₁₆, R₂₃, R₃₀, R₃₁, R₃₈, R₃₉, R₅₀, R₅₃=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Aspartate TREM Consensus Sequence

In an embodiment, a TREM disclosed herein comprises the sequence of Formula I ASP (SEQ ID NO: 571),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂ wherein R is a ribonucleotide residue and the consensus for Asp is:
R₀=absent
R₂₄, R₇₁=are independently A, C or absent;
R₃₃, R₄₆=are independently A, C, G or absent;
R₂, R₃, R₄, R₅, R₆, R₁₂, R₁₆, R₂₂, R₂₆, R₂₉, R₃₁, R₃₂, R₄₄, R₄₈, R₄₉, R₅₈, R₆₃, R₆₄, R₆₆, R₆₇, R₆₈, R₆₉=are
independently N or absent;
R₁₃, R₂₁, R₃₄, R₄₁, R₅₇, R₆₅=are independently A, C, U or absent;
R₉, R₁₀, R₁₄, R₁₅, R₂₀, R₂₇, R₃₇, R₄₀, R₅₁, R₅₆, R₇₂=are independently A, G or absent;
R₇, R₂₅, R₄₂=are independently A, G, U or absent;
R₃₉=C or absent;
R₅₀, R₆₂=are independently C, G or absent;
R₃₀, R₄₃, R₄₅, R₅₅, R₇₀=are independently C, G, U or absent;
R&, R₁₁, R₁₇, R₁₈, R₂₈, R₃₅, R₅₃, R₅₉, R₆₆, R₆₁=are independently C, U or absent;
R₁₉, R₅₂=are independently G or absent;
R₁=G, U or absent;
R₂₃, R₃₆, R₃₈, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II ASP (SEQ ID NO: 572),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Asp is:
R₀, R₁₇, R₁₈, R₂₃=are independently absent;
R₀, R₄₀=are independently A or absent;
R₂₄, R₇₁=are independently A, C or absent;
R₆₇, R₆₈=are independently A, C, G or absent;
R₂, R₆, R₆₆=are independently N or absent;
R₅₇, R₆₃=are independently A, C, U or absent;
R₁₀, R₁₄, R₂₇, R₃₃, R₃₇, R₄₄, R₄₆, R₅₁, R₅₆, R₆₄, R₇₂=are independently A, G or absent;
R₇, R₁₂, R₂₆, R₆₅=are independently A, U or absent;
R₃₉, R₆₁, R₆₂=are independently C or absent;
R₃, R₃₁, R₄₅, R₇₀=are independently C, G or absent;
R₄, R₅, R₂₉, R₄₃, R₅₅=are independently C, G, U or absent;
R₈, R₁₁, R₁₃, R₃₀, R₃₂, R₃₄, R₃₅, R₄₁, R₄₈, R₅₃, R₅₉, R₆₀=are independently C, U or absent;
R₁₅, R₁₉, R₂₀, R₂₅, R₄₂, R so, R₅₂=are independently G or absent;
R₁, R₂₂, R₄₉, R₅₈, R₆₉=are independently G, U or absent;
R₁₆, R₂₁, R₂₈, R₃₆, R₃₈, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III ASP (SEQ ID NO: 573),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Asp is:
R₀, R₁₇, R₁₈, R₂₃=are absent
R₉, R₁₂, R₄₀, R₆₅, R₇₁=are independently A or absent;
R₂, R₂₄, R₅₇=are independently A, C or absent;
R₆, R₁₄, R₂₇, R₄₆, R₅₁, R₅₆, R₆₄, R₆₇, R₆₈=are independently A, G or absent;
R₃, R₃₁, R₃₅, R₃₉, R₆₁, R₆₂=are independently C or absent;
R₆₆=C, G or absent;
R₅, R₈, R₂₉, R₃₀, R₃₂, R₃₄, R₄₁, R₄₃, R₄₈, R₅₅, R₅₉, R₆₀, R₆₃=are independently C, U or absent;
R₁₀, R₁₅, R₁₉, R₂₀, R₂₅, R₃₃, R₃₇, R₄₂, R₄₄, R₄₅, R₄₉, R₅₀, R₅₂, R₆₉, R₇₀, R₇₂=are independently G or absent;
R₂₂, R₅₅=are independently G, U or absent;
R₁, R₄, R₇, R₁₁, R₁₃, R₁₆, R₂₁, R₂₆, R₂₈, R₃₆, R₃₈, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Cysteine TREM Consensus Sequence

In an embodiment, a TREM disclosed herein comprises the sequence of Formula I cYs (SEQ ID NO: 574),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Cys is:
R₀=absent
R₁₄, R₃₉, R₅₇=are independently A or absent;
R₄₁=A, C or absent;
R₁₀, R₁₅, R₂₇, R₃₃, R₆₂=are independently A, C, G or absent;
R₃, R₄, R₅, R₆, R₁₂, R₁₃, R₁₆, R₂₄, R₂₆, R₂₉, R₃₀, R₃₁, R₃₂, R₃₄, R₄₂, R₄₄, R₄₅, R₄₆, R₄₈, R₄₉, R₅₈, R₆₃, R₆₄, R₆₆,
R₆₇, R₆₈, R₆₉, R₇₀=are independently N or absent;
R₆₅=A, C, U or absent;
R₉, R₂₅, R₃₇, R₄₀, R₅₂, R₅₆=are independently A, G or absent;
R₇, R₂₀, R₅₁=are independently A, G, U or absent;
R₁₈, R₃₈, R₅₅=are independently C or absent;
R₂=C, G or absent;
R₂₁, R₂₈, R₄₃, R₅₀=are independently C, G, U or absent;
R₁₁, R₂₂, R₂₃, R₃₅, R₃₆, R₅₉, R₆₀, R₆₁, R₇₁, R₇₂=are independently C, U or absent;
R₁, R₁₉=are independently G or absent;
R₁₇=G, U or absent;
R₅, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II CYs (SEQ ID NO: 575),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Cys is:
R₀, R₁₈, R₂₃=are absent;
R₁₄, R₂₄, R₂₆, R₂₉, R₃₉, R₄₁, R₄₅, R₅₇=are independently A or absent;
R₄₄=A, C or absent;
R₂₇, R₆₂=are independently A, C, G or absent;
R₁₆=A, C, G, U or absent;
R₃₀, R₇₀=are independently A, C, U or absent;
R₅, R₇, R₉, R₂₅, R₃₄, R₃₇, R₄₀, R₄₆, R₅₂, R₅₆, R₅₈, R₆₆=are independently A, G or absent;
R₂₀, R₅₁=are independently A, G, U or absent;
R₃₅, R₃₈, R₄₃, R₅₅, R₆₉=are independently C or absent;
R₂, R₄, R₁₅=are independently C, G or absent;
R₁₃=C, G, U or absent;
R₆, R₁₁, R₂₈, R₃₆, R₄₈, R₄₉, R so, R₆₀, R₆₁, R₆₇, R₆₈, R₇₁, R₇₂=are independently C, U or absent;
R₁, R₃, R₁₀, R₁₉, R₃₃, R₆₃=are independently G or absent;
R₈, R₁₇, R₂₁, R₆₄=are independently G, U or absent;
R₁₂, R₂₂, R₃₁, R₃₂, R₄₂, R₅₃, R₅₄, R₆₅=are independently U or absent;
R₅₉=U, or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III CYS (SEQ ID NO: 576),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Cys is:
R₀, R₁₈, R₂₃=are absent
R₁₄, R₂₄, R₂₆, R₂₉, R₃₄, R₃₉, R₄₁, R₄₅, R₅₇, R₅₅=are independently A or absent;
R₄₄, R₇₀=are independently A, C or absent;
R₆₂=A, C, G or absent;
R₁₆=N or absent;
R₅, R₇, R₉, R₂₀, R₄₀, R₄₆, R₅₁, R$2, R₅₆, R₆₆=are independently A, G or absent;
R₂₈, R₃₅, R₃₈, R₄₃, R₅₅, R₆₇, R₆₉=are independently C or absent;
R₄, R₁₅=are independently C, G or absent;
R₆, R₁₁, R₁₃, R₃₀, R₄₈, R₄₉, R₅₀, R₆₀, R₆₁, R₆₈, R₇₁, R₇₂=are independently C, U or absent;
R₁, R₂, R₃, R₁₀, R₁₉, R₂₅, R₂₇, R₃₃, R₃₇, R₆₃=are independently G or absent;
R₈, R₂₁, R₆₄=are independently G, U or absent;
R₁₂, R₁₇, R₂₂, R₃₁, R₃₂, R₃₆, R₄₂, R₅₃, R₅₄, R₅₉, R₆₅=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Glutamine TREM Consensus Sequence

In an embodiment, a TREM disclosed herein comprises the sequence of Formula I GLN (SEQ ID NO: 577),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Gln is:
R₀, R₁₈=are absent;
R₁₄, R₂₄, R₅₇=are independently A or absent;
R₉, R₂₆, R₂₇, R₃₃, R₅₆=are independently A, C, G or absent;
R₂, R₄, R₅, R₆, R₁₂, R₁₃, R₁₆, R₂₁, R₂₂, R₂₅, R₂₉, R₃₀, R₃₁, R₃₂, R₃₄, R₄₁, R₄₂, R₄₄, R₄₅, R₄₆, R₄₈, R₄₉, R₅₀, R₅₈, R₆₂, R₆₃, R₆₆, R₆₇, R₆₈, R₆₉, R₇₀=are independently N or absent;
R₁₇, R₂₃, R₄₃, R₆₅, R₇₁=are independently A, C, U or absent;
R₁₅, R₄₀, R₅₁, R₅₂=are independently A, G or absent;
R₁, R₇, R₇₂=are independently A, G, U or absent;
R₃, R₁₁, R₃₇, R₆₀, R₆₄=are independently C, G, U or absent;
R₂₈, R₃₅, R₅₅, R₅₉, R₆₁=are independently C, U or absent;
R₁₀, R₁₉, R₂₀=are independently G or absent;
R₃₉=G, U or absent;
R₈, R₃₆, R₃₈, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II GLN (SEQ ID NO: 578),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Gln is:
R₀, R₁₈, R₂₃=are absent
R₁₄, R₂₄, R₅₇=are independently A or absent;
R₁₇, R₇₁=are independently A, C or absent;
R₂₅, R₂₆, R₃₃, R₄₄, R₄₆, R₅₆, R₆₉=are independently A, C, G or absent;
R₄, R₅, R₁₂, R₂₂, R₂₉, R₃₀, R₄₈, R₄₉, R₆₃, R₆₇, R₆₈=are independently N or absent;
R₃₁, R₄₃, R₆₂, R₆₅, R₇₀=are independently A, C, U or absent;
R₁₅, R₂₇, R₃₄, R₄₀, R₄₁, R₅₁, R₅₂=are independently A, G or absent;
R₂, R₇, R₂₁, R₄₅, R₅₀, R₅₅, R₆₆, R₇₂=are independently A, G, U or absent;
R₃, R₁₃, R₃₂, R₃₇, R₄₂, R₆₀, R₆₄=are independently C, G, U or absent;
R₆, R₁₁, R₂₈, R₃₅, R₅₅, R₅₉, R₆₁=are independently C, U or absent;
R₉, R₁₀, R₁₉, R₂₀=are independently G or absent;
R₁, R₁₆, R₃₉=are independently G, U or absent;
R&, R₃₆, R₃₈, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III GLN (SEQ ID NO: 579),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Gln is:
R₀, R₁₈, R₂₃=are absent
R₁₄, R₂₄, R₄₁, R₅₇=are independently A or absent;
R₁₇, R₇₁=are independently A, C or absent;
R₅, R₂₅, R₂₆, R₄₆, R₅₆, R₆₉=are independently A, C, G or absent;
R₄, R₂₂, R₂₉, R₃₀, R₄₈, R₄₉, R₆₃, R₆₈=are independently N or absent;
R₄₃, R₆₂, R₆₅, R₇₀=are independently A, C, U or absent;
R₁₅, R₂₇, R₃₃, R₃₄, R₄₀, R₅₁, R₅₂=are independently A, G or absent;
R₂, R₇, R₁₂, R₄₅, R₅₀, R₅₅, R₆₆=are independently A, G, U or absent;
R₃₁=A, U or absent;
R₃₂, R₄₄, R₆₀=are independently C, G or absent;
R₃, R₁₃, R₃₇, R₄₂, R₆₄, R₆₇=are independently C, G, U or absent;
R₆, R₁₁, R₂₈, R₃₅, R₅₅, R₅₉, R₆₁=are independently C, U or absent;
R₉, R₁₀, R₁₉, R₂₀=are independently G or absent;
R₁, R₂₁, R₃₉, R₇₂=are independently G, U or absent;
R₈, R₁₆, R₃₆, R₃₈, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Glutamate TREM Consensus Sequence

In an embodiment, a TREM disclosed herein comprises the sequence of Formula I GLU (SEQ ID NO: 580),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Glu is:
R₀=absent;
R₃₄, R₄₃, R₆₈, R₆₉=are independently A, C, G or absent;
R₁, R₂, R₅, R₆, R₉, R₁₂, R₁₆, R₂₀, R₂₁, R₂₆, R₂₇, R₂₉, R₃₀, R₃₁, R₃₂, R₃₃, R₄₁, R₄₄, R₄₅, R₄₆, R₄₈, R so, R₅₁, R₅₈, R₆₃, R₆₄, R₆₅, R₆₆, R₇₀, R₇₁=are independently N or absent;
R₁₃, R₁₇, R₂₃, R₆₁=are independently A, C, U or absent;
R₁₀, R₁₄, R₂₄, R₄₀, R₅₂, R₅₆=are independently A, G or absent;
R₇, R₁₅, R₂₅, R₆₇, R₇₂=are independently A, G, U or absent;
R₁₁, R₅₇=are independently A, U or absent;
R₃₉=C, G or absent;
R₃, R₄, R₂₂, R₄₂, R₄₉, R₅₅, R₆₂=are independently C, G, U or absent;
R₁₈, R₂₈, R₃₅, R₃₇, R₅₃, R₅₉, R₆₀=are independently C, U or absent;
R₁₉=G or absent;
R₈, R₃₆, R₃₈, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II GLU (SEQ ID NO: 581),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Glu is:
R₀, R₁₈, R₂₃=are absent
R₁₇, R₄₀=are independently A or absent;
R₂₆, R₂₇, R₃₄, R₄₃, R₆₈, R₆₉, R₇₁=are independently A, C, G or absent;
R₁, R₂, R₅, R₁₂, R₂₁, R₃₁, R₃₃, R₄₁, R₄₅, R₄₈, R₅₁, R₅₈, R₆₆, R₇₀=are independently N or absent;
R₄₄, R₆₁=are independently A, C, U or absent;
R₉, R₁₄, R₂₄, R₂₅, R₅₂, R₅₆, R₆₃=are independently A, G or absent;
R₇, R₁₅, R₄₆, R₅₀, R₆₇, R₇₂=are independently A, G, U or absent;
R₂₉, R₅₇=are independently A, U or absent;
R₆₀=C or absent;
R₃₉=C, G or absent;
R₃, R₆, R₂₀, R₃₀, R₃₂, R₄₂, R₅₅, R₆₂, R₆₅=are independently C, G, U or absent;
R₄, R₈, R₁₆, R₂₈, R₃₅, R₃₇, R₄₉, R₅₃, R₅₉=are independently C, U or absent;
R₁₀, R₁₉=are independently G or absent;
R₂₂, R₆₄=are independently G, U or absent;
R₁₁, R₁₃, R₃₆, R₃₈, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III GLU (SEQ ID NO: 582),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Glu is:
R₀, R₁₇, R₁₈, R₂₃=are absent
R₁₄, R₂₇, R₄₀, R₇₁=are independently A or absent;
R₄₄=A, C or absent;
R₄₃=A, C, G or absent;
R₁, R₃₁, R₃₃, R₄₅, R₅₁, R₆₆=are independently N or absent;
R₂₁, R₄₁=are independently A, C, U or absent;
R₇, R₂₄, R₂₅, R₅₀, R₅₂, R₅₆, R₆₃, R₆₈, R₇₀=are independently A, G or absent;
R₅, R₄₆=are independently A, G, U or absent;
R₂₉, R₅₇, R₆₇, R₇₂=are independently A, U or absent;
R₂, R₃₉, R₆₀=are independently C or absent;
R₃, R₁₂, R₂₀, R₂₆, R₃₄, R₆₉=are independently C, G or absent;
R₆, R₃₀, R₄₂, R₄₈, R₆₅=are independently C, G, U or absent;
R₄, R₁₆, R₂₈, R₃₅, R₃₇, R₄₉, R₅₃, R₅₅, R₅₅, R₆₁, R₆₂=are independently C, U or absent;
R₀, R₁₀, R₁₉, R₆₄=are independently G or absent;
R₁₅, R₂₂, R₃₂=are independently G, U or absent;
R₈, R₁₁, R₁₃, R₃₆, R₃₈, R₅₄, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Glycine TREM Consensus Sequence

In an embodiment, a TREM disclosed herein comprises the sequence of Formula I GLY (SEQ ID NO: 583),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Gly is:
R₀=absent;
R₂₄=A or absent;
R₃, R₉, R₄₀, R₅₀, R₅₁=are independently A, C, G or absent;
R₄, R₅, R₆, R₇, R₁₂, R₁₆, R₂₁, R₂₂, R₂₆, R₂₉, R₃₀, R₃₁, R₃₂, R₃₃, R₃₄, R₄₁, R₄₂, R₄₃, R₄₄, R₄₅, R₄₆, R₄₈, R₄₉, R₅₈, R
63, R₆₄, R₆₅, R₆₆, R₆₇, R₆₈=are independently N or absent;
R₅₉=A, C, U or absent;
R₁, R₁₀, R₁₄, R₁₅, R₂₇, R₅₆=are independently A, G or absent;
R₂₀, R₂₅=are independently A, G, U or absent;
R₅₇, R₇₂=are independently A, U or absent;
R₃₈, R₃₉, R₆₀=are independently C or absent;
R₅₂=C, G or absent;
R₂, R₁₉, R₃₇, R₅₄, R₅₅, R₆₁, R₆₂, R₆₉, R₇₀=are independently C, G, U or absent;
R₁₁, R₁₃, R₁₇, R₂₈, R₃₅, R₃₆, R₇₁=are independently C, U or absent;
R₈, R₁₈, R₂₃, R₅₃=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II GLY (SEQ ID NO: 584),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Gly is:
R₀, R₁₈, R₂₃=are absent
R₂₄, R₂₇, R₄₀, R₇₂=are independently A or absent;
R₂₆=A, C or absent;
R₃, R₇, R₆₈=are independently A, C, G or absent;
R₅, R₃₀, R₄₁, R₄₂, R₄₄, R₄₉, R₆₇=are independently A, C, G, U or absent;
R₃₁, R₃₂, R₃₄=are independently A, C, U or absent;
R₀, R₁₀, R₁₄, R₁₅, R₃₃, R₅₀, R₅₆=are independently A, G or absent;
R₁₂, R₁₆, R₂₂, R₂₅, R₂₉, R₄₆=are independently A, G, U or absent;
R₅₇=A, U or absent;
R₁₇, R₃₈, R₃₉, R₆₀, R₆₁, R₇₁=are independently C or absent;
R₆, R₅₂, R₆₄, R₆₆=are independently C, G or absent;
R₂, R₄, R₃₇, R₄₈, R₅₅, R₆₅=are independently C, G, U or absent;
R₁₃, R₃₅, R₄₃, R₆₂, R₆₉=are independently C, U or absent;
R₁, R₁₉, R₂₀, R₅₁, R₇₀=are independently G or absent;
R₂₁, R₄₅, R₆₃=are independently G, U or absent;
R₈, R₁₁, R₂₈, R₃₆, R₅₃, R₅₄, R₅₈, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III GLY (SEQ ID NO: 585),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Gly is:
R₀, R₁₈, R₂₃=are absent
R₂₄, R₂₇, R₄₀, R₇₂=are independently A or absent;
R₂₆=A, C or absent;
R₃, R₇, R₄₉, R₆₈=are independently A, C, G or absent;
R₅, R₃₀, R₄₁, R₄₄, R₆₇=are independently N or absent;
R₃₁, R₃₂, R₃₄=are independently A, C, U or absent;
R₉, R₁₀, R₁₄, R₁₅, R₃₃, R₅₀, R₅₆=are independently A, G or absent;
R₁₂, R₂₅, R₂₉, R₄₂, R₄₆=are independently A, G, U or absent;
R₁₆, R₅₇=are independently A, U or absent;
R₁₇, R₃₈, R₃₉, R₆₀, R₆₁, R₇₁=are independently C or absent;
R₆, R₅₂, R₆₄, R₆₆=are independently C, G or absent;
R₃₇, R₄₈, R₆₅=are independently C, G, U or absent;
R₂, R₄, R₁₃, R₃₅, R₄₃, R₅₅, R₆₂, R₆₉=are independently C, U or absent;
R₁, R₁₉, R₂₀, R₅₁, R₇₀=are independently G or absent;
R₂₁, R₂₂, R₄₅, R₆₃=are independently G, U or absent;
R₈, R₁₁, R₂₈, R₃₆, R₅₃, R₅₄, R₅₈, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Histidine TREM Consensus Sequence

In an embodiment, a TREM disclosed herein comprises the sequence of Formula I HIS (SEQ ID NO: 586),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for His is:
R₂₃=absent;
R₁₄, R₂₄, R₅₇=are independently A or absent;
R₇₂=A, C or absent;
R₉, R₂₇, R₄₃, R₄₈, R₆₉=are independently A, C, G or absent;
R₃, R₄, R₅, R₆, R₁₂, R₂₅, R₂₆, R₂₉, R₃₀, R₃₁, R₃₄, R₄₂, R₄₅, R₄₆, R₄₉, R so, R₅₈, R₆₂, R₆₃, R₆₆, R₆₇, R₆₈=are independently N or absent;
R₁₃, R₂₁, R₄₁, R₄₄, R₆₅=are independently A, C, U or absent;
R₄₀, R₅₁, R₅₆, R₇₀=are independently A, G or absent;
R₇, R₃₂=are independently A, G, U or absent;
R₅₅, R₆₀=are independently C or absent;
R₁₁, R₁₆, R₃₃, R₆₄=are independently C, G, U or absent;
R₂, R₁₇, R₂₂, R₂₈, R₃₅, R₅₃, R₅₉, R₆₁, R₇₁=are independently C, U or absent;
R₁, R₁₀, R₁₅, R₁₉, R₂₀, R₃₇, R₃₉, R₅₂=are independently G or absent;
R₀=G, U or absent;
R₈, R₁₈, R₃₆, R₃₈, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II HIS (SEQ ID NO: 587),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for His is:
R₀, R₁₇, R₁₈, R₂₃=are absent;
R₇, R₁₂, R₁₄, R₂₄, R₂₇, R₄₅, R₅₇, R₅₈, R₆₃, R₆₇, R₇₂=are independently A or absent;
R₃=A, C, U or absent;
R₄, R₄₃, R₅₆, R₇₀=are independently A, G or absent;
R₄₉=A, U or absent;
R₂, R₂₈, R₃₀, R₄₁, R₄₂, R₄₄, R₄₈, R₅₅, R₆₀, R₆₆, R₇₁=are independently C or absent;
R₂₅=C, G or absent;
R₉=C, G, U or absent;
R₈, R₁₃, R₂₆, R₃₃, R₃₅, R₅₀, R₅₃, R₆₁, R₆₈=are independently C, U or absent;
R₁, R₆, R₁₀, R₁₅, R₁₉, R₂₀, R₃₂, R₃₄, R₃₇, R₃₉, R₄₀, R₄₆, R₅₁, R $2, R₆₂, R₆₄, R₆₉=are independently G or
absent;
R₁₆=G, U or absent;
R₅, R₁₁, R₂₁, R₂₂, R₂₉, R₃₁, R₃₆, R₃₈, R₅₄, R₅₉, R₆₅=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III HIS (SEQ ID NO: 588),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for His is:
R₀, R₁₇, R₁₈, R₂₃=are absent
R₇, R₁₂, R₁₄, R₂₄, R₂₇, R₄₅, R₅₇, R₅₈, R₆₃, R₆₇, R₇₂=are independently A or absent;
R₃=A, C or absent;
R₄, R₄₃, R₅₆, R₇₀=are independently A, G or absent;
R₄₉=A, U or absent;
R₂, R₂₈, R₃₀, R₄₁, R₄₂, R₄₄, R₄₈, R₅₅, R₆₀, R₆₆, R₇₁=are independently C or absent;
R₈, R₉, R₂₆, R₃₃, R₃₅, R₅₀, R₆₁, R₆₈=are independently C, U or absent;
R₁, R₆, R₁₀, R₁₅, R₁₉, R₂₀, R₂₅, R₃₂, R₃₄, R₃₇, R₃₉, R₄₀, R₄₆, R$1, R₅₂, R₆₂, R₆₄, R₆₉=are independently G
or absent;
R₅, R₁₁, R₁₃, R₁₆, R₂₁, R₂₂, R₂₉, R₃₁, R₃₆, R₃₈, R₅₃, R₅₄, R₅₉, R₆₅=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Isoleucine TREM Consensus Sequence

In an embodiment, a TREM disclosed herein comprises the sequence of Formula I ILE (SEQ ID NO: 589),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ile is:
R₂₃=absent;
R₃₈, R₄₁, R₅₇, R₇₂=are independently A or absent;
R₁, R₂₆=are independently A, C, G or absent;
R₀, R₃, R₄, R₆, R₁₆, R₃₁, R₃₂, R₃₄, R₃₇, R₄₂, R₄₃, R₄₄, R₄₅, R₄₆, R₄₈, R₄₉, Rso, R₅₈, R₅₉, R₆₂, R₆₃, R₆₄, R₆₆, R₆₇, R₆₈, R₆₉=are independently N or absent;
R₂₂, R₆₁, R₆₅=are independently A, C, U or absent;
R₉, R₁₄, R₁₅, R₂₄, R₂₇, R₄₀=are independently A, G or absent;
R₇, R₂₅, R₂₉, R₅₁, R₅₆=are independently A, G, U or absent;
R₁₈, R₅₄=are independently A, U or absent;
R₆₀=C or absent;
R₂, R₅₂, R₇₀=are independently C, G or absent;
R₅, R₁₂, R₂₁, R₃₀, R₃₃, R₇₁=are independently C, G, U or absent;
R₁₁, R₁₃, R₁₇, R₂₈, R₃₅, R₅₃, R₅₅=are independently C, U or absent;
R₁₀, R₁₉, R₂₀=are independently G or absent;
R₈, R₃₆, R₃₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II ILE (SEQ ID NO: 590),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ile is:
R₀, R₁₈, R₂₃=are absent
R₂₄, R₃₈, R₄₀, R₄₁, R₅₇, R₇₂=are independently A or absent;
R₂₆, R₆₅=are independently A, C or absent;
R₅₈, R₅₉, R₆₇=are independently N or absent;
R₂₂=A, C, U or absent;
R₆, R₉, R₁₄, R₁₅, R₂₉, R₃₄, R₄₃, R₄₆, R₄₈, R₅₀, R₅₁, R₆₃, R₆₉=are independently A, G or absent;
R₃₇, R₅₆=are independently A, G, U or absent;
R₅₄=A, U or absent;
R₂₈, R₃₅, R₆₀, R₆₂, R₇₁=are independently C or absent;
R₂, R₅₂, R₇₀=are independently C, G or absent;
R₅=C, G, U or absent;
R₃, R₄, R₁₁, R₁₃, R₁₇, R₂₁, R₃₀, R₄₂, R₄₄, R₄₅, R₄₉, R₅₃, R₅₅, R₆₁, R₆₄, R₆₆=are independently C, U or
absent;
R₁, R₁₀, R₁₉, R₂₀, R₂₅, R₂₇, R₃₁, R₆₈=are independently G or absent;
R₇, R₁₂, R₃₂=are independently G, U or absent;
R₈, R₁₆, R₃₃, R₃₆, R₃₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III ILE (SEQ ID NO: 591),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ile is:
R₀, R₁₈, R₂₃=are absent
R₁₄, R₂₄, R₃₈, R₄₀, R₄₁, R₅₇, R₇₂=are independently A or absent;
R₂₆, R₆₅=are independently A, C or absent;
R₂₂, R₅₉=are independently A, C, U or absent;
R₆, R₉, R₁₅, R₃₄, R₄₃, R₄₆, R₅₁, R₅₆, R₆₃, R₆₉=are independently A, G or absent;
R₃₇=A, G, U or absent;
R₁₃, R₂₈, R₃₅, R₄₄, R₅₅, R₆₀, R₆₂, R₇₁=are independently C or absent;
R₂, R₅, R₇₀=are independently C, G or absent;
R₅₅, R₆₇=are independently C, G, U or absent;
R₃, R₄, R₁₁, R₁₇, R₂₁, R₃₀, R₄₂, R₄₅, R₄₉, R₅₃, R₆₁, R₆₄, R₆₆=are independently C, U or absent;
R₁, R₁₀, R₁₉, R₂₀, R₂₅, R₂₇, R₂₉, R₃₁, R₃₂, R₄₈, R₅₀, R₅₂, R₆₈=are independently G or absent;
R₇, R₁₂=are independently G, U or absent;
R₅, R₁₆, R₃₃, R₃₆, R₃₉, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Methionine TREM Consensus Sequence

In an embodiment, a TREM disclosed herein comprises the sequence of Formula I MET (SEQ ID NO: 592),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Met is:
R₀, R₂₃=are absent;
R₁₄, R₃₈, R₄₀, R₅₇=are independently A or absent;
R₆₀=A, C or absent;
R₃₃, R₄₈, R₇₀=are independently A, C, G or absent;
R₁, R₃, R₄, R₅, R₆, R₁₁, R₁₂, R₁₆, R₁₇, R₂₁, R₂₂, R₂₆, R₂₇, R₂₉, R₃₀, R₃₁, R₃₂, R₄₂, R₄₄, R₄₅, R₄₆, R₄₉, R₅₀, R₅₈, R₆₂, R₆₃, R₆₆, R₆₇, R₆₈, R₆₉, R₇₁=are independently N or absent;
R₁₈, R₃₅, R₄₁, R₅₉, R₆₅=are independently A, C, U or absent;
R₉, R₁₅, R₅₁=are independently A, G or absent;
R₇, R₂₄, R₂₅, R₃₄, R₅₃, R₅₆=are independently A, G, U or absent;
R₇₂=A, U or absent;
R₃₇=C or absent;
R₁₀, R₅₅=are independently C, G or absent;
R₂, R₁₃, R₂₈, R₄₃, R₆₄=are independently C, G, U or absent;
R₃₆, R₆₁=are independently C, U or absent;
R₁₉, R₂₀, R₅₂=are independently G or absent;
R₈, R₃₉, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II MET (SEQ ID NO: 593),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Met is:
R₀, R₁₈, R₂₂, R₂₃=are absent
R₁₄, R₂₄, R₃₈, R₄₀, R₄₁, R₅₇, R₇₂=are independently A or absent;
R₅₉, R₆₀, R₆₂, R₆₅=are independently A, C or absent;
R₆, R₄₅, R₆₇=are independently A, C, G or absent;
R₄=N or absent;
R₂₁, R₄₂=are independently A, C, U or absent;
R₁, R₉, R₂₇, R₂₉, R₃₂, R₄₆, R₅₁=are independently A, G or absent;
R₁₇, R₄₉, R₅₃, R₅₆, R₅₅=are independently A, G, U or absent;
R₆₃=A, U or absent;
R₃, R₁₃, R₃₇=are independently C or absent;
R₄₈, R₅₅, R₆₄, R₇₀=are independently C, G or absent;
R₂, R₅, R₆₆, R₆₈=are independently C, G, U or absent;
R₁₁, R₁₆, R₂₆, R₂₈, R₃₀, R₃₁, R₃₅, R₃₆, R₄₃, R₄₄, R₆₁, R₇₁=are independently C, U or absent;
R₁₀, R₁₂, R₁₅, R₁₉, R₂₀, R₂₅, R₃₃, R₅₂, R₆₉=are independently G or absent;
R₇, R₃₄, R₅₀=are independently G, U or absent;
R₈, R₃₉, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III MET (SEQ ID NO: 594),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Met is:
R₀, R₁₈, R₂₂, R₂₃=are absent
R₁₄, R₂₄, R₃₈, R₄₀, R₄₁, R₅₇, R₇₂=are independently A or absent;
R₅₉, R₆₂, R₆₅=are independently A, C or absent;
R₆, R₆₇=are independently A, C, G or absent;
R₄, R₂₁=are independently A, C, U or absent;
R₁, R₉, R₂₇, R₂₉, R₃₂, R₄₅, R₄₆, R₅₁=are independently A, G or absent;
R₁₇, R₅₆, R₅₅=are independently A, G, U or absent;
R₄₉, R₅₃, R₆₃=are independently A, U or absent;
R₃, R₁₃, R₂₆, R₃₇, R₄₃, R₆₀=are independently C or absent;
R₂, R₄₈, R₅₅, R₆₄, R₇₀=are independently C, G or absent;
R₅, R₆₆=are independently C, G, U or absent;
R₁₁, R₁₆, R₂₈, R₃₀, R₃₁, R₃₅, R₃₆, R₄₂, R₄₄, R₆₁, R₇₁=are independently C, U or absent;
R₁₀, R₁₂, R₁₅, R₁₉, R₂₀, R₂₅, R₃₃, R₅₂, R₆₉=are independently G or absent;
R₇, R₃₄, R₅₀, R₆₈=are independently G, U or absent;
R₈, R₃₉, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Leucine TREM Consensus Sequence

In an embodiment, a TREM disclosed herein comprises the sequence of Formula I LEU (SEQ ID NO: 595),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Leu is:
R₀=absent;
R₃₈, R₅₇=are independently A or absent;
R₆₀=A, C or absent;
R₁, R₁₃, R₂₇, R₄₈, R₅₁, R₅₆=are independently A, C, G or absent;
R₂, R₃, R₄, R₅, R₆, R₇, R₀, R₁₀, R₁₁, R₁₂, R₁₆, R₂₃, R₂₆, R₂₈, R₂₉, R₃₀, R₃₁, R₃₂, R₃₃, R₃₄, R₃₇, R₄₁, R₄₂, R₄₃, R₄₄, R₄₅, R₄₆, R₄₉, R₅₀, R₅₈, R₆₂, R₆₃, R₆₅, R₆₆, R₆₇, R₆₈, R₆₉, R₇₀=are independently N or absent;
R₁₇, R₁₈, R₂₁, R₂₂, R₂₅, R₃₅, R₅₅=are independently A, C, U or absent;
R₁₄, R₁₅, R₃₉, R₇₂=are independently A, G or absent;
R₂₄, R₄₀=are independently A, G, U or absent;
R₅₂, R₆₁, R₆₄, R₇₁=are independently C, G, U or absent;
R₃₆, R₅₃, R₅₉=are independently C, U or absent;
R₁₉=G or absent;
R₂₀=G, U or absent;
R₅, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II LEU (SEQ ID NO: 596),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Leu is:
R₀=absent
R₃₈, R₅₇, R₇₂=are independently A or absent;
R₆₀=A, C or absent;
R₄, R₅, R₄₈, R so, R₅₆, R₆₉=are independently A, C, G or absent;
R₆, R₃₃, R₄₁, R₄₃, R₄₆, R₄₉, R₅₈, R₆₃, R₆₆, R₇₀=are independently N or absent;
R₁₁, R₁₂, R₁₇, R₂₁, R₂₂, R₂₈, R₃₁, R₃₇, R₄₄, R₅₅=are independently A, C, U or absent;
R₁, R₉, R₁₄, R₁₅, R₂₄, R₂₇, R₃₄, R₃₉=are independently A, G or absent;
R₇, R₂₉, R₃₂, R₄₀, R₄₅=are independently A, G, U or absent;
R₂₅=A, U or absent;
R₁₃=C, G or absent;
R₂, R₃, R₁₆, R₂₆, R₃₀, R₅₂, R₆₂, R₆₄, R₆₅, R₆₇, R₆₈=are independently C, G, U or absent;
R₁₈, R₃₅, R₄₂, R₅₃, R₅₉, R₆₁, R₇₁=are independently C, U or absent;
R₁₉, R₅₁=are independently G or absent;
R₁₀, R₂₀=are independently G, U or absent;
R₈, R₂₃, R₃₆, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III LEU (SEQ ID NO: 597),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Leu is:
R₀=absent
R₃₈, R₅₇, R₇₂=are independently A or absent;
R₆₀=A, C or absent;
R₄, R₅, R₄₈, R so, R₅₆, R₅₈, R₆₉=are independently A, C, G or absent;
R₆, R₃₃, R₄₃, R₄₆, R₄₉, R₆₃, R₆₆, R₇₀=are independently N or absent;
R₁₁, R₁₂, R₁₇, R₂₁, R₂₂, R₂₈, R₃₁, R₃₇, R₄₁, R₄₄, R₅₅=are independently A, C, U or absent;
R₁, R₉, R₁₄, R₁₅, R₂₄, R₂₇, R₃₄, R₃₉=are independently A, G or absent;
R₇, R₂₉, R₃₂, R₄₀, R₄₅=are independently A, G, U or absent;
R₂₅=A, U or absent;
R₁₃=C, G or absent;
R₂, R₃, R₁₆, R₃₀, R₅₂, R₆₂, R₆₄, R₆₇, R₆₈=are independently C, G, U or absent;
R₁₈, R₃₅, R₄₂, R₅₃, R₅₉, R₆₁, R₆₅, R₇₁=are independently C, U or absent;
R₁₉, R₅₁=are independently G or absent;
R₁₀, R₂₀, R₂₆=are independently G, U or absent;
R₈, R₂₃, R₃₆, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Lysine TREM Consensus Sequence

In an embodiment, a TREM disclosed herein comprises the sequence of Formula I LYS (SEQ ID NO: 598),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Lys is:
R₀=absent
R₁₄=A or absent;
R₄₀, R₄₁=are independently A, C or absent;
R₃₄, R₄₃, R₅₁=are independently A, C, G or absent;
R₁, R₂, R₃, R₄, R₅, R₆, R₇, R₁₁, R₁₂, R₁₆, R₂₁, R₂₆, R₃₀, R₃₁, R₃₂, R₄₄, R₄₅, R₄₆, R₄₈, R₄₉, R so, R₅₈, R₆₂, R₆₃, R₆₅, R₆₆, R₆₇, R₆₈, R₆₉, R₇₀=are independently N or absent;
R₁₃, R₁₇, R₅₉, R₇₁=are independently A, C, U or absent;
R₉, R₁₅, R₁₉, R₂₀, R₂₅, R₂₇, R₅₂, R₅₆=are independently A, G or absent;
R₂₄, R₂₉, R₇₂=are independently A, G, U or absent;
R₁₈, R₅₇=are independently A, U or absent;
R₁₀, R₃₃=are independently C, G or absent;
R₄₂, R₆₁, R₆₄=are independently C, G, U or absent;
R₂₈, R₃₅, R₃₆, R₃₇, R₅₃, R₅₅, R₆₀=are independently C, U or absent;
R₈, R₂₂, R₂₃, R₃₈, R₃₉, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II LYS (SEQ ID NO: 599),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Lys is:
R₀, R₁₈, R₂₃=are absent
R₁₄=A or absent;
R₄₀, R₄₁, R₄₃=are independently A, C or absent;
R₃, R₇=are independently A, C, G or absent;
R₁, R₆, R₁₁, R₃₁, R₄₅, R₄₈, R₄₉, R₆₃, R₆₅, R₆₆, R₆₈=are independently N or absent;
R₂, R₁₂, R₁₃, R₁₇, R₄₄, R₆₇, R₇₁=are independently A, C, U or absent;
R₉, R₁₅, R₁₉, R₂₀, R₂₅, R₂₇, R₃₄, R₅₀, R₅₂, R₅₆, R₇₀, R₇₂=are independently A, G or absent;
R₅, R₂₄, R₂₆, R₂₉, R₃₂, R₄₆, R₆₉=are independently A, G, U or absent;
R₅₇=A, U or absent;
R₁₀, R₆₁=are independently C, G or absent;
R₄, R₁₆, R₂₁, R₃₀, R₅₈, R₆₄=are independently C, G, U or absent;
R₂₈, R₃₅, R₃₆, R₃₇, R₄₂, R₅₃, R₅₅, R₅₉, R₆₀, R₆₂=are independently C, U or absent;
R₃₃, R₅₁=are independently G or absent;
R₈=G, U or absent;
R₂₂, R₃₈, R₃₉, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III LYS (SEQ ID NO: 600),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Lys is:
R₀, R₁₈, R₂₃=absent
R₉, R₁₄, R₃₄, R₄₁=are independently A or absent;
R₄₀=A, C or absent;
R₁, R₃, R₇, R₃₁=are independently A, C, G or absent;
R₄₈, R₆₅, R₆₈=are independently N or absent;
R₂, R₁₃, R₁₇, R₄₄, R₆₃, R₆₆=are independently A, C, U or absent;
R₅, R₁₅, R₁₉, R₂₀, R₂₅, R₂₇, R₂₉, R so, R $2, R₅₆, R₇₀, R₇₂=are independently A, G or absent;
R₆, R₂₄, R₃₂, R₄₉=are independently A, G, U or absent;
R₁₂, R₂₆, R₄₆, R₅₇=are independently A, U or absent;
R₁₁, R₂₈, R₃₅, R₄₃=are independently C or absent;
R₁₀, R₄₅, R₆₁=are independently C, G or absent;
R₄, R₂₁, R₆₄=are independently C, G, U or absent;
R₃₇, R₅₃, R₅₅, R₅₉, R₆₀, R₆₂, R₆₇, R₇₁=are independently C, U or absent;
R₃₃, R₅₁=are independently G or absent;
R₈, R₃₀, R₅₅, R₆₉=are independently G, U or absent;
R₁₆, R₂₂, R₃₆, R₃₈, R₃₉, R₄₂, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Phenylalanine TREM Consensus Sequence

In an embodiment, a TREM disclosed herein comprises the sequence of Formula I PHE (SEQ ID NO: 601),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Phe is:
R₀, R₂₃=are absent
R₉, R₁₄, R₃₈, R₃₉, R₅₇, R₇₂=are independently A or absent;
R₇₁=A, C or absent;
R₄₁, R₇₀=are independently A, C, G or absent;
R₄, R₅, R₆, R₃₀, R₃₁, R₃₂, R₃₄, R₄₂, R₄₄, R₄₅, R₄₆, R₄₈, R₄₉, R₅₈, R₆₂, R₆₃, R₆₆, R₆₇, R₆₈, R₆₉=are
independently N or absent;
R₁₆, R₆₁, R₆₅=are independently A, C, U or absent;
R₁₅, R₂₆, R₂₇, R₂₉, R₄₀, R₅₆=are independently A, G or absent;
R₇, R₅₁=are independently A, G, U or absent;
R₂₂, R₂₄=are independently A, U or absent;
R₅₅, R₆₀=are independently C or absent;
R₂, R₃, R₂₁, R₃₃, R₄₃, R₅₀, R₆₄=are independently C, G, U or absent;
R₁₁, R₁₂, R₁₃, R₁₇, R₂₈, R₃₅, R₃₆, R₅₉=are independently C, U or absent;
R₁₀, R₁₉, R₂₀, R₂₅, R₃₇, R₅₂=are independently G or absent;
R₁=G, U or absent;
R₈, R₁₈, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II PHE (SEQ ID NO: 602),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Phe is:
R₀, R₁₈, R₂₃=absent
R₁₄, R₂₄, R₃₈, R₃₉, R₅₇, R₇₂=are independently A or absent;
R₄₆, R₇₁=are independently A, C or absent;
R₄, R₇₀=are independently A, C, G or absent;
R₄₅=A, C, U or absent;
R₆, R₇, R₁₅, R₂₆, R₂₇, R₃₂, R₃₄, R₄₀, R₄₁, R₅₆, R₆₉=are independently A, G or absent;
R₂₉=A, G, U or absent;
R₅, R₉, R₆₇=are independently A, U or absent;
R₃₅, R₄₉, R₅₅, R₆₀=are independently C or absent;
R₂₁, R₄₃, R₆₂=are independently C, G or absent;
R₂, R₃₃, R₆₈=are independently C, G, U or absent;
R₃, R₁₁, R₁₂, R₁₃, R₂₈, R₃₀, R₃₆, R₄₂, R₄₄, R₄₈, R₅₈, R₅₉, R₆₁, R₆₆=are independently C, U or absent;
R₁₀, R₁₉, R₂₀, R₂₅, R₃₇, R₅₁, R₅₂, R₆₃, R₆₄=are independently G or absent;
R₁, R₃₁, R₅₀=are independently G, U or absent;
R₈, R₁₆, R₁₇, R₂₂, R₅₃, R₅₄, R₆₅=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III PHE (SEQ ID NO: 603),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Phe is:
R₀, R₁₈, R₂₂, R₂₃=absent
R₅, R₇, R₁₄, R₂₄, R₂₆, R₃₂, R₃₄, R₃₈, R₃₉, R₄₁, R₅₇, R₇₂=are independently A or absent;
R₄₆=A, C or absent;
R₇₀=A, C, G or absent;
R₄, R₆, R₁₅, R₅₆, R₆₉=are independently A, G or absent;
R₀, R₄₅=are independently A, U or absent;
R₂, R₁₁, R₁₃, R₃₅, R₄₃, R₄₉, R₅₅, R₆₀, R₆₈, R₇₁=are independently C or absent;
R₃₃=C, G or absent;
R₃, R₂₈, R₃₆, R₄₈, R₅₈, R₅₉, R₆₁=are independently C, U or absent;
R₁, R₁₀, R₁₉, R₂₀, R₂₁, R₂₅, R₂₇, R₂₉, R₃₇, R₄₀, R$1, R₅₂, R₆₂, R₆₃, R₆₄=are independently G or absent;
R₈, R₁₂, R₁₆, R₁₇, R₃₀, R₃₁, R₄₂, R₄₄, R₅₀, R₅₃, R₅₄, R₆₅, R₆₆, R₆₇=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Proline TREM Consensus Sequence

In an embodiment, a TREM disclosed herein comprises the sequence of Formula I PRO (SEQ ID NO: 604),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Pro is:
R₀=absent
R₁₄, R₅₇=are independently A or absent;
R₇₀, R₇₂=are independently A, C or absent;
R₉, R₂₆, R₂₇=are independently A, C, G or absent;
R₄, R₅, R₆, R₁₆, R₂₁, R₂₉, R₃₀, R₃₁, R₃₂, R₃₃, R₃₄, R₃₇, R₄₁, R₄₂, R₄₃, R₄₄, R₄₅, R₄₆, R₄₈, R₄₉, R so, R₅₈, R₆₁, R₆₂,
R₆₃, R₆₄, R₆₆, R₆₇, R₆₈=are independently N or absent;
R₃₅, R₆₅=are independently A, C, U or absent;
R₂₄, R₄₀, R₅₆=are independently A, G or absent;
R₇, R₂₅, R₅₁=are independently A, G, U or absent;
R₅₅, R₆₀=are independently C or absent;
R₁, R₃, R₇₁=are independently C, G or absent;
R₁₁, R₁₂, R₂₀, R₆₉=are independently C, G, U or absent;
R₁₃, R₁₇, R₁₈, R₂₂, R₂₃, R₂₈, R₅₉=are independently C, U or absent;
R₁₀, R₁₅, R₁₉, R₃₈, R₃₉, R₅₂=are independently G or absent;
R₂=are independently G, U or absent;
R₈, R₃₆, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II PRO (SEQ ID NO: 605),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Pro is:
R₀, R₁₇, R₁₈, R₂₂, R₂₃-absent;
R₁₄, R₄₅, R₅₆, R₅₇, R₅₅, Ros, R₆₈=are independently A or absent;
R₆₁=A, C, G or absent;
R₄₃=N or absent;
R₃₇=A, C, U or absent;
R₂₄, R₂₇, R₃₃, R₄₀, R₄₄, R₆₃=are independently A, G or absent;
R₃, R₁₂, R₃₀, R₃₂, R₄₈, R₅₅, R₆₀, R₇₀, R₇₁, R₇₂=are independently C or absent;
R₅, R₃₄, R₄₂, R₆₆=are independently C, G or absent;
R₂₀=C, G, U or absent;
R₃₅, R₄₁, R₄₉, R₆₂=are independently C, U or absent;
R₁, R₂, R₆, R₀, R₁₀, R₁₅, R₁₉, R₂₆, R₃₈, R₃₉, R₄₆, R so, R₅₁, R $2, R₆₄, R₆₇, R₆₉=are independently G or absent;
R₁₁, R₁₆=are independently G, U or absent;
R₄, R₇, R₈, R₁₃, R₂₁, R₂₅, R₂₈, R₂₉, R₃₁, R₃₆, R₅₃, R₅₄, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III PRO (SEQ ID NO: 606),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Pro is:
R₀, R₁₇, R₁₈, R₂₂, R₂₃=absent
R₁₄, R₄₅, R₅₆, R₅₇, R₅₈, R₆₅, R₆₈=are independently A or absent;
R₃₇=A, C, U or absent;
R₂₄, R₂₇, R₄₀=are independently A, G or absent;
R₃, R₅, R₁₂, R₃₀, R₃₂, R₄₈, R₄₉, R₅₅, R₆₀, R₆₁, R₆₂, R₆₆, R₇₀, R₇₁, R₇₂=are independently C or absent;
R₃₄, R₄₂=are independently C, G or absent;
R₄₃=C, G, U or absent;
R₄₁=C, U or absent;
R₁, R₂, R₆, R₉, R₁₀, R₁₅, R₁₉, R₂₀, R₂₆, R₃₃, R₃₈, R₃₉, R₄₄, R₄₆, R₅₀, R₅₁, R₅₂, R₆₃, R₆₄, R₆₇, R₆₉=are independently G or absent;
R₁₆=G, U or absent;
R₄, R₇, R₈, R₁₁, R₁₃, R₂₁, R₂₅, R₂₈, R₂₉, R₃₁, R₃₅, R₃₆, R₅₃, R₅₄, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Serine TREM Consensus Sequence

In an embodiment, a TREM disclosed herein comprises the sequence of Formula I SER (SEQ ID NO: 607),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ser is:
R₀=absent;
R₁₄, R₂₄, R₅₇=are independently A or absent;
R₄₁=A, C or absent;
R₂, R₃, R₄, R₅, R₆, R₇, R₀, R₁₀, R₁₁, R₁₂, R₁₃, R₁₆, R₂₁, R₂₅, R₂₆, R₂₇, R₂₈, R₃₀, R₃₁, R₃₂, R₃₃, R₃₄, R₃₇, R₄₂, R₄₃, R₄₄, R₄₅, R₄₆, R₄₈, R₄₉, R₅₀, R₆₂, R₆₃, R₆₄, R₆₅, R₆₆, R₆₇, R₆₈, R₆₉, R₇₀=are independently N or absent;
R₁₈=A, C, U or absent;
R₁₅, R₄₀, R₅₁, R₅₆=are independently A, G or absent;
R₁, R₂₉, R₅₅, R₇₂=are independently A, G, U or absent;
R₃₉=A, U or absent;
R₆₀=C or absent;
R₃₈=C, G or absent;
R₁₇, R₂₂, R₂₃, R₇₁=are independently C, G, U or absent;
R₈, R₃₅, R₃₆, R₅₅, R₅₉, R₆₁=are independently C, U or absent;
R₁₉, R₂₀=are independently G or absent;
R₅₂=G, U or absent;
R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II SER (SEQ ID NO: 608),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ser is:
R₀, R₂₃=absent
R₁₄, R₂₄, R₄₁, R₅₇=are independently A or absent;
R₄₄=A, C or absent;
R₂₅, R₄₅, R₄₈=are independently A, C, G or absent;
R₂, R₃, R₄, R₅, R₃₇, R₅₀, R₆₂, R₆₆, R₆₇, R₆₉, R₇₀=are independently N or absent;
R₁₂, R₂₈, R₆₅=are independently A, C, U or absent;
R₉, R₁₅, R₂₉, R₃₄, R₄₀, R₅₆, R₆₃=are independently A, G or absent;
R₇, R₂₆, R₃₀, R₃₃, R₄₆, R₅₅, R₇₂=are independently A, G, U or absent;
R₃₉=A, U or absent;
R₁₁, R₃₅, R₆₀, R₆₁=are independently C or absent;
R₁₃, R₃₈=are independently C, G or absent;
R₆, R₁₇, R₃₁, R₄₃, R₆₄, R₆₈=are independently C, G, U or absent;
R₃₆, R₄₂, R₄₉, R₅₅, R₅₉, R₇₁=are independently C, U or absent;
R₁₀, R₁₉, R₂₀, R₂₇, R₅₁=are independently G or absent;
R₁, R₁₆, R₃₂, R₅₂=are independently G, U or absent;
R₈, R₁₈, R₂₁, R₂₂, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III SER (SEQ ID NO: 609),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ser is:
R₀, R₂₃=absent
R₁₄, R₂₄, R₄₁, R₅₇, R₅₅=are independently A or absent;
R₄₄=A, C or absent;
R₂₅, R₄₈=are independently A, C, G or absent;
R₂, R₃, R₅, R₃₇, R₆₆, R₆₇, R₆₉, R₇₀=are independently N or absent;
R₁₂, R₂₈, R₆₂=are independently A, C, U or absent;
R₇, R₉, R₁₅, R₂₉, R₃₃, R₃₄, R₄₀, R₄₅, R₅₆, R₆₃=are independently A, G or absent;
R₄, R₂₆, R₄₆, R₅₀=are independently A, G, U or absent;
R₃₀, R₃₉=are independently A, U or absent;
R₁₁, R₁₇, R₃₅, R₆₀, R₆₁=are independently C or absent;
R₁₃, R₃₈=are independently C, G or absent;
R₆, R₆₄=are independently C, G, U or absent;
R₃₁, R₄₂, R₄₃, R₄₉, R₅₅, R₅₉, R₆₅, R₆₈, R₇₁=are independently C, U or absent;
R₁₀, R₁₉, R₂₀, R₂₇, R₅₁, R₅₂=are independently G or absent;
R₁, R₁₆, R₃₂, R₇₂=are independently G, U or absent;
R₈, R₁₈, R₂₁, R₂₂, R₃₆, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Threonine TREM Consensus Sequence

In an embodiment, a TREM disclosed herein comprises the sequence of Formula I_{THR} (SEQ ID NO: 610),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Thr is:
R₀, R₂₃=absent
R₁₄, R₄₁, R₅₇=are independently A or absent;
R₅₆, R₇₀=are independently A, C, G or absent;
R₄, R₅, R₆, R₇, R₁₂, R₁₆, R₂₆, R₃₀, R₃₁, R₃₂, R₃₄, R₃₇, R₄₂, R₄₄, R₄₅, R₄₆, R₄₈, R₄₉, R₅₀, R₅₈, R₆₂, R₆₃, R₆₄, R₆₅, R₆₆, R₆₇, R₆₈, R₇₂=are independently N or absent;
R₁₃, R₁₇, R₂₁, R₃₅, R₆₁=are independently A, C, U or absent;
R₁, R₉, R₂₄, R₂₇, R₂₉, R₆₉=are independently A, G or absent;
R₁₅, R₂₅, R₅₁=are independently A, G, U or absent;
R₄₀, R₅₃=are independently A, U or absent;
R₃₃, R₄₃=are independently C, G or absent;
R₂, R₃, R₅₉=are independently C, G, U or absent;
R₁₁, R₁₈, R₂₂, R₂₈, R₃₆, R₅₄, R₅₅, R₆₀, R₇₁=are independently C, U or absent;
R₁₀, R₂₀, R₃₈, R₅₂=are independently G or absent;
R₁₉=G, U or absent;
R₈, R₃₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II THR (SEQ ID NO: 611),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Thr is:
R₀, R₁₈, R₂₃=absent
R₁₄, R₄₁, R₅₇=are independently A or absent;
R₉, R₄₂, R₄₄, R₄₈, R₅₆, R₇₀=are independently A, C, G or absent;
R₄, R₆, R₁₂, R₂₆, R₄₉, R₅₈, R₆₃, R₆₄, R₆₆, R₆₈=are independently N or absent;
R₁₃, R₂₁, R₃₁, R₃₇, R₆₂=are independently A, C, U or absent;
R₁, R₁₅, R₂₄, R₂₇, R₂₉, R₄₆, R₅₁, R₆₉=are independently A, G or absent;
R₇, R₂₅, R₄₅, R₅₀, R₆₇=are independently A, G, U or absent;
R₄₀, R₅₃=are independently A, U or absent;
R₃₅=C or absent;
R₃₃, R₄₃=are independently C, G or absent;
R₂, R₃, R₅, R₁₆, R₃₂, R₃₄, R₅₉, R₆₅, R₇₂=are independently C, G, U or absent;
R₁₁, R₁₇, R₂₂, R₂₈, R₃₀, R₃₆, R₅₅, R₆₀, R₆₁, R₇₁=are independently C, U or absent;
R₁₀, R₁₉, R₂₀, R₃₈, R₅₂=are independently G or absent;
R₈, R₃₉, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III THR (SEQ ID NO: 612),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Thr is:
R₀, R₁₈, R₂₃=absent
R₁₄, R₄₀, R₄₁, R₅₇=are independently A or absent;
R₄₄=A, C or absent;
R₉, R₄₂, R₄₈, R₅₆=are independently A, C, G or absent;
R₄, R₆, R₁₂, R₂₆, R₅₅, R₆₄, R₆₆, R₆₈=are independently N or absent;
R₁₃, R₂₁, R₃₁, R₃₇, R₄₉, R₆₂=are independently A, C, U or absent;
R₁, R₁₅, R₂₄, R₂₇, R₂₉, R₄₆, R₅₁, R₆₉=are independently A, G or absent;
R₇, R₂₅, R₄₅, R₅₀, R₆₃, R₆₇=are independently A, G, U or absent;
R₅₃=A, U or absent;
R₃₅=C or absent;
R₂, R₃₃, R₄₃, R₇₀=are independently C, G or absent;
R₅, R₁₆, R₃₄, R₅₉, R₆₅=are independently C, G, U or absent;
R₃, R₁₁, R₂₂, R₂₈, R₃₀, R₃₆, R₅₅, R₆₀, R₆₁, R₇₁=are independently C, U or absent;
R₁₀, R₁₉, R₂₀, R₃₈, R₅₂=are independently G or absent;
R₃₂=G, U or absent;
R₈, R₁₇, R₃₉, R₅₄, R₇₂=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Tryptophan TREM Consensus Sequence

In an embodiment, a TREM disclosed herein comprises the sequence of Formula I TRP (SEQ ID NO: 613),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Trp is:
R₀=absent;
R₂₄, R₃₉, R₄₁, R₅₇=are independently A or absent;
R₂, R₃, R₂₆, R₂₇, R₄₀, R₄₈=are independently A, C, G or absent;
R₄, R₅, R₆, R₂₉, R₃₀, R₃₁, R₃₂, R₃₄, R₄₂, R₄₄, R₄₅, R₄₆, R₄₉, R₅₁, R₅₈, R₆₃, R₆₆, R₆₇, R₆₈=are independently N or absent;
R₁₃, R₁₄, R₁₆, R₁₈, R₂₁, R₆₁, R₆₅, R₇₁=are independently A, C, U or absent;
R₁, R₉, R₁₀, R₁₅, R₃₃, R₅₀, R₅₆=are independently A, G or absent;
R₇, R₂₅, R₇₂=are independently A, G, U or absent;
R₃₇, R₃₈, R₅₅, R₆₀=are independently C or absent;
R₁₂, R₃₅, R₄₃, R₆₄, R₆₉, R₇₀=are independently C, G, U or absent;
R₁₁, R₁₇, R₂₂, R₂₈, R₅₉, R₆₂=are independently C, U or absent;
R₁₉, R₂₀, R₅₂=are independently G or absent;
R₈, R₂₃, R₃₆, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II TRP (SEQ ID NO: 614),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Trp is:
R₀, R₁₈, R₂₂, R₂₃=absent
R₁₄, R₂₄, R₃₉, R₄₁, R₅₇, R₇₂=are independently A or absent;
R₃, R₄, R₁₃, R₆₁, R₇₁=are independently A, C or absent;
R₆, R₄₄=are independently A, C, G or absent;
R₂₁=A, C, U or absent;
R₂, R₇, R₁₅, R₂₅, R₃₃, R₃₄, R₄₅, R₅₆, R₆₃=are independently A, G or absent;
R₅₅=A, G, U or absent;
R₄₆=A, U or absent;
R₃₇, R₃₈, R₅₅, R₆₀, R₆₂=are independently C or absent;
R₁₂, R₂₆, R₂₇, R₃₅, R₄₀, R₄₈, R₆₇=are independently C, G or absent;
R₃₂, R₄₃, R₆₈=are independently C, G, U or absent;
R₁₁, R₁₆, R₂₈, R₃₁, R₄₉, R₅₉, R₆₅, R₇₀=are independently C, U or absent;
R₁, R₉, R₁₀, R₁₉, R₂₀, R₅₀, R₅₂, R₆₉=are independently G or absent;
R₅, R₈, R₂₉, R₃₀, R₄₂, R₅₁, R₆₄, R₆₆=are independently G, U or absent;
R₁₇, R₃₆, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III TRP (SEQ ID NO: 615),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Trp is:
R₀, R₁₈, R₂₂, R₂₃=absent
R₁₄, R₂₄, R₃₉, R₄₁, R₅₇, R₇₂=are independently A or absent;
R₃, R₄, R₁₃, R₆₁, R₇₁=are independently A, C or absent;
R₆, R₄₄=are independently A, C, G or absent;
R₂₁=A, C, U or absent;
R₂, R₇, R₁₅, R₂₅, R₃₃, R₃₄, R₄₅, R₅₆, R₆₃=are independently A, G or absent;
R₅₅=A, G, U or absent;
R₄₆=A, U or absent;
R₃₇, R₃₈, R₅₅, R₆₀, R₆₂=are independently C or absent;
R₁₂, R₂₆, R₂₇, R₃₅, R₄₀, R₄₈, R₆₇=are independently C, G or absent;
R₃₂, R₄₃, R₆₈=are independently C, G, U or absent;
R₁₁, R₁₆, R₂₈, R₃₁, R₄₉, R₅₉, R₆₅, R₇₀=are independently C, U or absent;
R₁, R₉, R₁₀, R₁₉, R₂₀, R₅₀, R₅₂, R₆₉=are independently G or absent;
R₅, R₈, R₂₉, R₃₀, R₄₂, R₅₁, R₆₄, R₆₆=are independently G, U or absent;
R₁₇, R₃₆, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Tyrosine TREM Consensus Sequence

In an embodiment, a TREM disclosed herein comprises the sequence of Formula I TYR (SEQ ID NO: 616),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Tyr is:
R₀=absent
R₁₄, R₃₉, R₅₇=are independently A or absent;
R₄₁, R₄₈, R₅₁, R₇₁=are independently A, C, G or absent;
R₃, R₄, R₅, R₆, R₉, R₁₀, R₁₂, R₁₃, R₁₆, R₂₅, R₂₆, R₃₀, R₃₁, R₃₂, R₄₂, R₄₄, R₄₅, R₄₆, R₄₉, R₅₀, R₅₈, R₆₂, R₆₃, R₆₆, R₆₇, R₆₈, R₆₉, R₇₀=are independently N or absent;
R₂₂, R₆₅=are independently A, C, U or absent;
R₁₅, R₂₄, R₂₇, R₃₃, R₃₇, R₄₀, R₅₆=are independently A, G or absent;
R₇, R₂₉, R₃₄, R₇₂=are independently A, G, U or absent;
R₂₃, R₅₃=are independently A, U or absent;
R₃₅, R₆₀=are independently C or absent;
R₂₀=C, G or absent;
R₁, R₂, R₂₈, R₆₁, R₆₄=are independently C, G, U or absent;
R₁₁, R₁₇, R₂₁, R₄₃, R₅₅=are independently C, U or absent;
R₁₉, R₅₂=are independently G or absent;
R₈, R₁₈, R₃₆, R₃₈, R₅₄, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II TYR (SEQ ID NO: 617),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Tyr is:
R₀, R₁₈, R₂₃=absent
R₇, R₉, R₁₄, R₂₄, R₂₆, R₃₄, R₃₉, R₅₇=are independently A or absent;
R₄₄, R₆₉=are independently A, C or absent;
R₇₁=A, C, G or absent;
R₆₈=N or absent;
R₅₈=A, C, U or absent;
R₃₃, R₃₇, R₄₁, R₅₆, R₆₂, R₆₃=are independently A, G or absent;
R₆, R₂₉, R₇₂=are independently A, G, U or absent;
R₃₁, R₄₅, R₅₃=are independently A, U or absent;
R₁₃, R₃₅, R₄₉, R₆₀=are independently C or absent;
R₂₀, R₄₈, R₆₄, R₆₇, R₇₀=are independently C, G or absent;
R₁, R₂, R₅, R₁₆, R₆₆=are independently C, G, U or absent;
R₁₁, R₂₁, R₂₈, R₄₃, R₅₅, R₆₁=are independently C, U or absent;
R₁₀, R₁₅, R₁₉, R₂₅, R₂₇, R₄₀, R₅₁, R₅₂=are independently G or absent;
R₃, R₄, R₃₀, R₃₂, R₄₂, R₄₆=are independently G, U or absent;
R₈, R₁₂, R₁₇, R₂₂, R₃₆, R₃₈, R₅₀, R₅₄, R₅₉, R₆₅=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III TYR (SEQ ID NO: 618),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Tyr is:
R₀, R₁₈, R₂₃=absent
R₇, R₉, R₁₄, R₂₄, R₂₆, R₃₄, R₃₉, R₅₇, R₇₂=are independently A or absent;
R₄₄, R₆₉=are independently A, C or absent;
R₇₁=A, C, G or absent;
R₃₇, R₄₁, R₅₆, R₆₂, R₆₃=are independently A, G or absent;
R₆, R₂₉, R₆₈=are independently A, G, U or absent;
R₃₁, R₄₅, R₅₅=are independently A, U or absent;
R₁₃, R₂₈, R₃₅, R₄₉, R₆₀, R₆₁=are independently C or absent;
R₅, R₄₈, R₆₄, R₆₇, R₇₀=are independently C, G or absent;
R₁, R₂=are independently C, G, U or absent;
R₁₁, R₁₆, R₂₁, R₄₃, R₅₅, R₆₆=are independently C, U or absent;
R₁₀, R₁₅, R₁₉, R₂₀, R₂₅, R₂₇, R₃₃, R₄₀, R₅₁, R₅₂=are independently G or absent;
R₃, R₄, R₃₀, R₃₂, R₄₂, R₄₆=are independently G, U or absent;
R₈, R₁₂, R₁₇, R₂₂, R₃₆, R₃₈, R₅₀, R₅₃, R₅₄, R₅₉, R₆₅=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Valine TREM Consensus Sequence

In an embodiment, a TREM disclosed herein comprises the sequence of Formula I VAL (SEQ ID NO: 619),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Val is:
R₀, R₂₃=absent;
R₂₄, R₃₈, R₅₇=are independently A or absent;
R₀, R₇₂=are independently A, C, G or absent;
R₂, R₄, R₅, R₆, R₇, R₁₂, R₁₅, R₁₆, R₂₁, R₂₅, R₂₆, R₂₉, R₃₁, R₃₂, R₃₃, R₃₄, R₃₇, R₄₁, R₄₂, R₄₃, R₄₄, R₄₅, R₄₆, R₄₈, R₄₉, R₅₀, R₅₈, R₆₁, R₆₂, R₆₃, R₆₄, R₆₅, R₆₆, R₆₇, R₆₈, R₆₉, R₇₀=are independently N or absent;
R₁₇, R₃₅, R₅₉=are independently A, C, U or absent;
R₁₀, R₁₄, R₂₇, R₄₀, R₅₂, R₅₆=are independently A, G or absent;
R₁, R₃, R₅₁, R₅₃=are independently A, G, U or absent;
R₃₉=C or absent;
R₁₃, R₃₀, R₅₅=are independently C, G, U or absent;
R₁₁, R₂₂, R₂₈, R₆₀, R₇₁=are independently C, U or absent;
R₁₉=G or absent;
R₂₀=G, U or absent;
R₈, R₁₈, R₃₆, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II VAL (SEQ ID NO: 620),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Val is:
R₀, R₁₈, R₂₃=absent;
R₂₄, R₃₈, R₅₇=are independently A or absent;
R₆₄, R₇₀, R₇₂=are independently A, C, G or absent;
R₁₅, R₁₆, R₂₆, R₂₉, R₃₁, R₃₂, R₄₃, R₄₄, R₄₅, R₄₉, R so, R₅₈, R₆₂, R₆₅=are independently N or absent;
R₆, R₁₇, R₃₄, R₃₇, R₄₁, R₅₉=are independently A, C, U or absent;
R₀, R₁₀, R₁₄, R₂₇, R₄₀, R₄₆, R$1, R₅₂, R₅₆=are independently A, G or absent;
R₇, R₁₂, R₂₅, R₃₃, R₅₃, R₆₃, R₆₆, R₆₈=are independently A, G, U or absent;
R₆₉=A, U or absent;
R₃₉=C or absent;
R₅, R₆₇=are independently C, G or absent;
R₂, R₄, R₁₃, R₄₈, R₅₅, R₆₁=are independently C, G, U or absent;
R₁₁, R₂₂, R₂₈, R₃₀, R₃₅, R₆₀, R₇₁=are independently C, U or absent;
R₁₉=G or absent;
R₁, R₃, R₂₀, R₄₂=are independently G, U or absent;
R₈, R₂₁, R₃₆, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III VAL (SEQ ID NO: 621),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Val is:
R₀, R₁₈, R₂₃=absent
R₂₄, R₃₈, R₄₀, R₅₇, R₇₂=are independently A or absent;
R₂₉, R₆₄, R₇₀=are independently A, C, G or absent;
R₄₉, R₅₀, R₆₂=are independently N or absent;
R₁₆, R₂₆, R₃₁, R₃₂, R₃₇, R₄₁, R₄₃, R₅₉, R₆₅=are independently A, C, U or absent;
R₉, R₁₄, R₂₇, R₄₆, R₅₂, R₅₆, R₆₆=are independently A, G or absent;
R₇, R₁₂, R₂₅, R₃₃, R₄₄, R₄₅, R₅₃, R₅₈, R₆₃, R₆₈=are independently A, G, U or absent;
R₆₉=A, U or absent;
R₃₉=C or absent;
R₅, R₆₇=are independently C, G or absent;
R₂, R₄, R₁₃, R₁₅, R₄₈, R₅₅=are independently C, G, U or absent;
R₆, R₁₁, R₂₂, R₂₈, R₃₀, R₃₄, R₃₅, R₆₀, R₆₁, R₇₁=are independently C, U or absent;
R₁₀, R₁₉, R₅₁=are independently G or absent;
R₁, R₃, R₂₀, R₄₂=are independently G, U or absent;
R₈, R₁₇, R₂₁, R₃₆, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Variable Region Consensus Sequence

In an embodiment, a TREM disclosed herein comprises a variable region at position R₄₇. In an embodiment, the variable region is 1-271 ribonucleotides in length (e.g. 1-250, 1-225, 1-200, 1-175, 1-150, 1-125, 1-100, 1-75, 1-50, 1-40, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, 1-10, 10-271, 20-271, 30-271, 40-271, 50-271, 60-271, 70-271, 80-271, 100-271, 125-271, 150-271, 175-271, 200-271, 225-271, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 110, 125, 150, 175, 200, 225, 250, or 271 ribonucleotides). In an embodiment, the variable region comprises any one, all or a combination of Adenine, Cytosine, Guanine or Uracil.

In an embodiment, the variable region comprises a ribonucleic acid (RNA) sequence encoded by a deoxyribonucleic acid (DNA) sequence disclosed in Table 4, e.g., any one of SEQ ID NOs: 452-561 disclosed in Table 4.

TABLE 4
Exemplary variable region sequences.

| | SEQ ID NO | SEQUENCE |
|---|---|---|
| 1 | 452 | AAAATATAAATATATTTC |
| 2 | 453 | AAGCT |
| 3 | 454 | AAGTT |
| 4 | 455 | AATTCTTCGGAATGT |
| 5 | 456 | AGA |
| 6 | 457 | AGTCC |
| 7 | 458 | CAACC |
| 8 | 459 | CAATC |
| 9 | 460 | CAGC |
| 10 | 461 | CAGGCGGGTTCTGCCCGCGC |
| 11 | 462 | CATACCTGCAAGGGTATC |
| 12 | 463 | CGACCGCAAGGTTGT |
| 13 | 464 | CGACCTTGCGGTCAT |
| 14 | 465 | CGATGCTAATCACATCGT |
| 15 | 466 | CGATGGTGACATCAT |
| 16 | 467 | CGATGGTTTACATCGT |
| 17 | 468 | CGCCGTAAGGTGT |
| 18 | 469 | CGCCTTAGGTGT |
| 19 | 470 | CGCCTTTCGACGCGT |
| 20 | 471 | CGCTTCACGGCGT |
| 21 | 472 | CGGCAGCAATGCTGT |
| 22 | 473 | CGGCTCCGCCTTC |
| 23 | 474 | CGGGTATCACAGGGTC |
| 24 | 475 | CGGTGCGCAAGCGCTGT |
| 25 | 476 | CGTACGGGTGACCGTACC |
| 26 | 477 | CGTCAAAGACTTC |
| 27 | 478 | CGTCGTAAGACTT |
| 28 | 479 | CGTTGAATAAACGT |
| 29 | 480 | CTGTC |
| 30 | 481 | GGCC |
| 31 | 482 | GGGGATT |
| 32 | 483 | GGTC |
| 33 | 484 | GGTTT |
| 34 | 485 | GTAG |
| 35 | 486 | TAACTAGATACTTTCAGAT |
| 36 | 487 | TACTCGTATGGGTGC |
| 37 | 488 | TACTTTGCGGTGT |
| 38 | 489 | TAGGCGAGTAACATCGTGC |
| 39 | 490 | TAGGCGTGAATAGCGCCTC |
| 40 | 491 | TAGGTCGCGAGAGCGGCGC |
| 41 | 492 | TAGGTCGCGTAAGCGGCGC |
| 42 | 493 | TAGGTGGTTATCCACGC |
| 43 | 494 | TAGTC |
| 44 | 495 | TAGTT |
| 45 | 496 | TATACGTGAAAGCGTATC |
| 46 | 497 | TATAGGGTCAAAAACTCTATC |
| 47 | 498 | TATGCAGAAATACCTGCATC |
| 48 | 499 | TCCCCATACGGGGGC |
| 49 | 500 | TCCCGAAGGGGTTC |
| 50 | 50 | TCTACGTATGTGGGC |
| 51 | 502 | TCTCATAGGAGTTC |
| 52 | 503 | TCTCCTCTGGAGGC |
| 53 | 504 | TCTTAGCAATAAGGT |
| 54 | 505 | TCTTGTAGGAGTTC |
| 55 | 506 | TGAACGTAAGTTCGC |
| 56 | 507 | TGAACTGCGAGGTTCC |
| 57 | 508 | TGAC |
| 58 | 509 | TGACCGAAAGGTCGT |
| 59 | 510 | TGACCGCAAGGTCGT |
| 60 | 511 | TGAGCTCTGCTCTC |
| 61 | 512 | TGAGGCCTCACGGCCTAC |
| 62 | 513 | TGAGGGCAACTTCGT |
| 63 | 514 | TGAGGGTCATACCTCC |
| 64 | 515 | TGAGGGTGCAAATCCTCC |
| 65 | 516 | TGCCGAAAGGCGT |
| 66 | 517 | TGCCGTAAGGCGT |
| 67 | 518 | TGCGGTCTCCGCGC |
| 68 | 519 | TGCTAGAGCAT |
| 69 | 520 | TGCTCGTATAGAGCTC |
| 70 | 521 | TGGACAATTGTCTGC |
| 71 | 522 | TGGACAGATGTCCGT |
| 72 | 523 | TGGACAGGTGTCCGC |
| 73 | 524 | TGGACGGTTGTCCGC |
| 74 | 525 | TGGACTTGTGGTC |
| 75 | 526 | TGGAGATTCTCTCCGC |
| 76 | 527 | TGGCATAGGCCTGC |
| 77 | 528 | TGGCTTATGTCTAC |
| 78 | 529 | TGGGAGTTAATCCCGT |
| 79 | 530 | TGGGATCTTCCCGC |
| 80 | 531 | TGGGCAGAAATGTCTC |
| 81 | 532 | TGGGCGTTCGCCCGC |
| 82 | 533 | TGGGCTTCGCCCGC |
| 83 | 534 | TGGGGGATAACCCCGT |
| 84 | 535 | TGGGGGTTTCCCCGT |
| 85 | 536 | TGGT |
| 86 | 537 | TGGTGGCAACACCGT |
| 87 | 538 | TGGTTTATAGCCGT |
| 88 | 539 | TGTACGGTAATACCGTACC |
| 89 | 540 | TGTCCGCAAGGACGT |
| 90 | 541 | TGTCCTAACGGACGT |
| 91 | 542 | TGTCCTATTAACGGACGT |
| 92 | 543 | TGTCCTTCACGGGCGT |
| 93 | 544 | TGTCTTAGGACGT |
| 94 | 545 | TGTGCGTTAACGCGTACC |
| 95 | 546 | TGTGTCGCAAGGCACC |
| 96 | 547 | TGTTCGTAAGGACTT |
| 97 | 548 | TTCACAGAAATGTGTC |
| 98 | 549 | TTCCCTCGTGGAGT |
| 99 | 550 | TTCCCTCTGGGAGC |
| 100 | 551 | TTCCCTTGTGGATC |
| 101 | 552 | TTCCTTCGGGAGC |
| 102 | 553 | TTCTAGCAATAGAGT |
| 103 | 554 | TTCTCCACTGGGGAGC |
| 104 | 555 | TTCTCGAGAGGGAGC |
| 105 | 556 | TTCTCGTATGAGAGC |
| 106 | 557 | TTTAAGGTTTTCCCTTAAC |
| 107 | 558 | TTTCATTGTGGAGT |
| 108 | 559 | TTTCGAAGGAATCC |
| 109 | 560 | TTTCTTCGGAAGC |
| 110 | 561 | TTTGGGGCAACTCAAC |

### Corresponding Nucleotide Positions

To determine if a selected nucleotide position in a candidate sequence corresponds to a selected position in a reference sequence (e.g., SEQ ID NO: 622, SEQ ID NO: 623, SEQ ID NO: 624), one or more of the following Evaluations is performed.

### Evaluation A:

1. The candidate sequence is aligned with each of the consensus sequences in Tables 9 and 10. The consensus sequence(s) having the most positions aligned (and which has at least 60% of the positions of the candidate sequence aligned) is selected.

The alignment is performed as is follows. The candidate sequence and an isodecoder consensus sequence from Tables 10A-10B are aligned based on a global pairwise alignment calculated with the Needleman-Wunsch algorithm when run with match scores from Table 11, a mismatch penalty of −1, a gap opening penalty of −1, and a gap extension penalty of −0.5, and no penalty for end gaps. The alignment with the highest overall alignment score is then used to determine the percent similarity between the candidate and the consensus sequence by counting the number of matched positions in the alignment, dividing it by the larger of the number of non-N bases in the candidate sequence or the consensus sequence, and multiplying the result by 100. In cases where multiple alignments (of the candidate and a single consensus sequence) tie for the same score, the percent similarity is the largest percent similarity calculated from the tied alignments. This process is repeated for the candidate sequence with each of the remaining isodecoder consensus sequences in Tables 10A-10B, and the alignment resulting in the greatest percent similarity is selected. If this alignment has a percent similarity equal to or greater than 60%, it is considered a valid alignment and used to relate positions in the candidate sequence to those in the consensus sequence, otherwise the candidate sequence is considered to have not aligned to any of the isodecoder consensus sequences. If there is a tie at this point, all tied consensus sequences are taken forward to step 2 in the analysis.

2. Using the selected consensus sequence(s) from step 1, one determines the consensus sequence position number that aligns with the selected position (e.g., a modified position) in the candidate sequence. One then assigns the position number of the aligned position in the consensus sequence to the selected position in the candidate sequence, in other words, the selected position in the candidate sequence is numbered according to the numbering of the consensus sequence. If there were tied consensus sequences from step one, and they give different position numbers in this step 2, then all such position numbers are taken forward to step 5.

3. The reference sequence is aligned with the consensus sequence chosen in step 1. The alignment is performed as described in step 1.

4. From the alignment in step 3, one determines the consensus sequence position number that aligns with the selected position (e.g., a modified position) in the reference sequence. One then assigns the position number of the aligned position in the consensus sequence to the selected position in the reference sequence, in other words, the selected position in the reference sequence is numbered according to the numbering of the consensus sequence. If there is a tie at this point, all tied consensus sequences are taken forward to step 5 in the analysis.

5. If a value for a position number determined for the reference sequence in step 2 is the same as the value for the position number determined for the candidate sequence in step 4, the positions are defined as corresponding.

### Evaluation B:

The reference sequence (e.g., a TREM sequence described herein) and the candidate sequence are aligned with one another. The alignment is performed as follows.

The reference sequence and the candidate sequence are aligned based on a global pairwise alignment calculated with the Needleman-Wunsch algorithm when run with match scores from Table 11, a mismatch penalty of −1, a gap opening penalty of −1, and a gap extension penalty of −0.5, and no penalty for end gaps. The alignment with the highest overall alignment score is then used to determine the percent similarity between the candidate and reference sequence by counting the number of matched based in the alignment, dividing it by the larger of the number of non-N bases in the candidate or reference sequence, and multiplying the result by 100. In cases where multiple alignments tie for the same score, the percent similarity is the largest percent similarity calculated from the tied alignments. If this alignment has a percent similarity equal to or greater than 60%, it is considered a valid alignment and used to relate positions in the candidate sequence to those in the reference sequence, otherwise the candidate sequence is considered to have not aligned to the reference sequence.

If the selected nucleotide position in the reference sequence (e.g., a modified position) is paired with a selected nucleotide position (e.g., a modified position) in the candidate sequence, the positions are defined as corresponding.

### Evaluation C:

The candidate sequence is assigned a nucleotide position number according to the comprehensive tRNA numbering system (CtNS), also referred to as the tRNAviz method (e.g., as described in Lin et al., Nucleic Acids Research, 47: W1, pages W542-W547, 2 Jul. 2019), which serves as a global numbering system for tRNA molecules. The alignment is performed as follows.

1. The candidate sequence is assigned a nucleotide position according to the tRNAviz method. For a novel sequence not present in the tRNAviz database, the numbering for the closest sequence in the database is obtained. For example, if a TREM differs at any given nucleotide position from a sequence in the database, the numbering for the tRNA having the wildtype sequence at said given nucleotide position is used.

2. The reference sequence is assigned a nucleotide position according to the method described in 1.

3. If a value for a position number determined for the reference sequence in step 1 is the same as the value for the position number determined for the candidate sequence in step 2, the positions are defined as corresponding.

If the selected position in the reference sequence and the candidate sequence are found to be corresponding in at least one of Evaluations A, B, and C, the positions correspond. For example, if two positions are found to be corresponding under Evaluation A, but do not correspond under Evaluation B or Evaluation C, the positions are defined as corresponding. Similarly, if two positions are found to be corresponding under Evaluation B, but do not correspond under Evaluation A or Evaluation C, the positions are defined as corresponding. In addition, if two positions are found to be corresponding under Evaluation C, but do not correspond under Evaluation A or Evaluation B, the positions are defined as corresponding

The numbering given above is used for ease of presentation and does not imply a required sequence. If more than one Evaluation is performed, they can be performed in any order.

TABLE 10A
Consensus sequence computationally generated for each isodecoder
by aligning members of the isodecoder family

| SEQ ID | Amino | | |
|---|---|---|---|
| NO. | Acid | Anticodon | Consensus sequence |
| 1200 | Ala | AGC | GGGGAATTAGCTCAAGTGGTAGAGCGCTTG |
| | | | CTTAGCATGCAAGAGGTAGTGGGATCGATG |
| | | | CCCACATTCTCCA |
| 1201 | Ala | CGC | GGGGATGTAGCTCAGTGGTAGAGCGCATGC |
| | | | TTCGCATGTATGAGGTCCCGGGTTCGATCCC |
| | | | CGGCATCTCCA |
| 1202 | Ala | TGC | GGGGGTGTAGCTCAGTGGTAGAGCGCATGC |
| | | | TTTGCATGTATGAGGCCCCGGGTTCGATCCC |
| | | | CGGCACCTCCA |
| 1203 | Arg | ACG | GGGCCAGTGGCGCAATGGATAACGCGTCTG |
| | | | ACTACGGATCAGAAGATTCCAGGTTCGACTC |
| | | | CTGGCTGGCTCG |
| 1204 | Arg | CCG | GGCCGCGTGGCCTAATGGATAAGGCGTCTG |
| | | | ATTCCGGATCAGAAGATTGAGGGTTCGAGTC |
| | | | CCTTCGTGGTCG |
| 1205 | Arg | CCT | GCCCCAGTGGCCTAATGGATAAGGCACTGG |
| | | | CCTCCTAAGCCAGGGATTGTGGGTTCGAGTC |
| | | | CCACCTGGGGTA |
| 1206 | Arg | TCG | GACCGCGTGGCCTAATGGATAAGGCGTCTG |
| | | | ACTTCGGATCAGAAGATTGAGGGTTCGAGTC |
| | | | CCTCCGTGGTCG |
| 1207 | Arg | TCT | GGCTCTGTGGCGCAATGGATNAGCGCATTG |
| | | | GACTTCTAATTCAAAGGTTGCGGGTTCGAGT |
| | | | CCCNCCAGAGTCG |
| 1208 | Asn | GTT | GTCTCTGTGGCGCAATCGGTTAGCGCGTTCG |
| | | | GCTGTTAACCGNAAAGGTTGGTGGTTCGAGC |
| | | | CCACCCAGGGACG |
| 1209 | Asp | GTC | TCCTCGTTAGTATAGTGGTGAGTATCCCCGC |
| | | | CTGTCACGCGGGAGACCGGGGTTCGATTCCC |
| | | | CGACGGGGAG |
| 1210 | Cys | GCA | GGGGGTATAGCTCAGNGGGTAGAGCATTTG |
| | | | ACTGCAGATCAAGAGGTCCCCGGTTCAAATC |
| | | | CGGGTGCCCCCT |
| 1211 | Gln | CTG | GGTTCCATGGTGTAATGGTNAGCACTCTGGA |
| | | | CTCTGAATCCAGCGATCCGAGTTCAAGTCTC |
| | | | GGTGGAACCT |
| 1212 | Gln | TTG | GGTCCCATGGTGTAATGGTTAGCACTCTGGA |
| | | | CTTTGAATCCAGCGATCCGAGTTCAAATCTC |
| | | | GGTGGGACCT |
| 1213 | Glu | CTC | TCCCTGGTGGTCTAGTGGTTAGGATTCGGCG |
| | | | CTCTCACCGCCGCGGCCCGGGTTCGATTCCC |
| | | | GGTCAGGGAA |
| 1214 | Glu | TTC | TCCCTGGTGGTCTAGTGGCTAGGATTCGGCG |
| | | | CTTTCACCGCNGCGGCCCGGGTTCGATTCCC |
| | | | GGTCAGGGAA |
| 1215 | Gly | CCC | GCATTGGTGGTTCAGTGGTAGAATTCTCGCC |
| | | | TCCCACGCNGGAGACCCGGGTTCGATTCCCG |
| | | | GCCAATGCA |
| 1216 | Gly | GCC | GCATTGGTGGTTCAGTGGTAGAATTCTCGCC |
| | | | TGCCACGCGGGAGGCCCGGGTTCGATTCCCG |
| | | | GCCAATGCA |
| 1217 | Gly | TCC | GCGTTGGTGGTATAGTGGTGAGCATAGCTGC |
| | | | CTTCCAAGCAGTTGACCCGGGTTCGATTCCC |
| | | | GGCCAACGCA |
| 1218 | Ile | AAT | GGCCGGTTAGCTCAGTTGGTTAGAGCGTGGT |
| | | | GCTAATAACGCCAAGGTCGCGGGTTCGATCC |
| | | | CCGTACGGGCCA |
| 1219 | Ile | TAT | GCTCCAGTGGCGCAATCGGTTAGCGCGCGGT |
| | | | ACTTATAATGCCGAGGTTGTGAGTTCGAGCC |
| | | | TCACCTGGAGCA |
| 1220 | Leu | AAG | GGTAGCGTGGCCGAGCGGTCTAAGGCGCTG |
| | | | GATTAAGGCTCCAGTCTCTTCGGGGGCGTGG |
| | | | GTTCGAATCCCACCGCTGCCA |
| 1221 | Leu | CAA | GTCAGGATGGCCGAGTGGTCNTAAGGCGCC |
| | | | AGACTCAAGTTCTGGTCTCCGNATGGAGGCG |
| | | | TGGGTTCGAATCCCACTTCTGACA |
| 1222 | Leu | CAG | GTCAGGATGGCCGAGCGGTCTAAGGCGCTG |
| | | | CGTTCAGGTCGCAGTCTCCCCTGGAGGCGTG |
| | | | GGTTCGAATCCCACTCCTGACA |
| 1223 | Leu | TAA | ACCAGGATGGCCGAGTGGTTAAGGCGTTGG |
| | | | ACTTAAGATCCAATGGACAGATGTCCGCGTG |
| | | | GGTTCGAACCCCACTCCTGGTA |
| 1224 | Leu | TAG | GGTAGCGTGGCCGAGCGGTCTAAGGCGCTG |
| | | | GATTTAGGCTCCAGTCTCTTCGGNGGCGTGG |
| | | | GTTCGAATCCCACCGCTGCCA |
| 1225 | Lys | CTT | GCCCGGCTAGCTCAGTCGGTAGAGCATGAG |
| | | | ACTCTTAATCTCAGGGTCGTGGGTTCGAGCC |
| | | | CCACGTTGGGCGNNN |
| 1226 | Lys | TTT | GCCTGGATAGCTCAGTCGGTAGAGCATCAG |
| | | | ACTTTTAATCTGAGGGTCCAGGGTTCAAGTC |
| | | | CCTGTTCAGGCG |
| 1227 | Met | CAT | GCCCTCTTAGCGCAGTNGGCAGCGCGTCAGT |
| | | | CTCATAATCTGAAGGTCCTGAGTTCGAGCCT |
| | | | CAGAGAGGGCA |
| 1228 | Phe | GAA | GCCGAAATAGCTCAGTTGGGAGAGCGTTAG |
| | | | ACTGAAGATCNTAAAGGTCCCTGGTTCAATC |
| | | | CCGGGTTTCGGCA |
| 1229 | Pro | AGG | GGCTCGTTGGTCTAGGGGTATGATTCTCGCT |
| | | | TAGGATGCGAGAGGTCCCGGGTTCAAATCC |
| | | | CGGACGAGCCC |
| 1230 | Pro | CGG | GGCTCGTTGGTCTAGGGGTATGATTCTCGCT |
| | | | TCGGGTGCGAGAGGTCCCGGGTTCAAATCCC |
| | | | GGACGAGCCC |
| 1231 | Pro | TGG | GGCTCGTTGGTCTAGGGGTATGATTCTCGCT |
| | | | TTGGGTGCGAGAGGTCCCGGGTTCAAATCCC |
| | | | GGACGAGCCC |
| 1232 | Ser | AGA | GTAGTCGTGGCCGAGTGGTTAAGGCGATGG |
| | | | ACTAGAAATCCATTGGGGTTTCCCCGCGCAG |
| | | | GTTCGAATCCTGCCGACTACG |
| 1233 | Ser | CGA | GCTGTGATGGCCGAGTGGTTAAGGCGTTGG |
| | | | ACTCGAAATCCAATGGGGTCTCCCCGCGCAG |
| | | | GTTCGAATCCTGCTCACAGCG |
| 1234 | Ser | GCT | GACGAGGNNTGGCCGAGTGGTTAAGGCGAT |
| | | | GGACTGCTAATCCATTGTGCTCTGCACGCGT |
| | | | GGGTTCGAATCCCATCCTCGTCG |
| 1235 | Ser | TGA | GTAGTCGTGGCCGAGTGGTTAAGGCGATGG |
| | | | ACTTGAAATCCATTGGGGTCTCCCCGCGCAG |
| | | | GTTCGAATCCTGCCGGCTACG |
| 1236 | Thr | AGT | GGCTCCGTGGCTTAGCTGGTTAAAGCGCCTG |
| | | | TCTAGTAAACAGGAGATCCTGGGTTCGAATC |
| | | | CCAGCGGGGCCT |
| 1237 | Thr | CGT | GGCNCTGTGGCTNAGTNGGNTAAAGCGCCG |
| | | | GTCTCGTAAACCNGGAGATCNTGGGTTCGA |
| | | | ATCCCANCNGGGCCT |
| 1238 | Thr | TGT | GGCTCCATAGCTCAGNGGGTTAGAGCACTG |
| | | | GTCTTGTAAACCAGGGGTCGCGAGTTCAAAT |
| | | | CTCGCTGGGGCCT |
| 1239 | Trp | CCA | GACCTCGTGGCGCAACGGTAGCGCGTCTGA |
| | | | CTCCAGATCAGAAGGTTGCGTGTTCAAATCA |
| | | | CGTCGGGGTCA |
| 1240 | Tyr | GTA | CCTTCGATAGCTCAGCTGGTAGAGCGGAGG |
| | | | ACTGTAGATCCTTAGGTCGCTGGTTCGATTC |
| | | | CGGCTCGAAGGA |
| 1241 | Val | AAC | GTTTCCGTAGTGTAGTGGTTATCACGTTCGC |
| | | | CTAACACGCGAAAGGTCCCCGGTTCGAAAC |
| | | | CGGGCGGAAACA |
| 1242 | Val | CAC | GTTTCCGTAGTGTAGTGGTTATCACGTTCGC |
| | | | CTCACACGCGAAAGGTCCCCGGTTCGAAAC |
| | | | CGGGCGGAAACA |
| 1243 | Val | TAC | GGTTCCATAGTGTAGTGGTTATCACGTCTGC |
| | | | TTTACACGCAGAAGGTCCTGGGTTCGAGCCC |
| | | | CAGTGGAACCA |
| 1244 | iMet | CAT | AGCAGAGTGGCGCAGCGGAAGCGTGCTGGG |
| | | | CCCATAACCCAGAGGTCGATGGATCGAAAC |
| | | | CATCCTCTGCTA |

TABLE 10B
Consensus sequence computationally generated for each isodecoder
by aligning members of the isodecoder family

| SEQ ID | Amino | | |
|---|---|---|---|
| NO | Acid | Anticodon | Consensus sequence |
| 1245 | Ala | AGC | GGGGAATTAGCTCAAGTGGTAGAGCGCTTGC |
| | | | TTAGCATGCAAGAGGTAGTGGGATCGATGCC |
| | | | CACATTCTCCANNN |
| 1246 | Ala | CGC | GGGGATGTAGCTCAGTGGTAGAGCGCATGCT |
| | | | TCGCATGTATGAGGTCCCGGGTTCGATCCCC |
| | | | GGCATCTCCANNN |
| 1247 | Ala | TGC | GGGGGTGTAGCTCAGTGGTAGAGCGCATGCT |
| | | | TTGCATGTATGAGGCCCCGGGTTCGATCCCC |
| | | | GGCACCTCCANNN |
| 1248 | Arg | ACG | GGGCCAGTGGCGCAATGGATAACGCGTCTGA |
| | | | CTACGGATCAGAAGATTCCAGGTTCGACTCC |
| | | | TGGCTGGCTCGNNN |
| 1249 | Arg | CCG | GGCCGCGTGGCCTAATGGATAAGGCGTCTGA |
| | | | TTCCGGATCAGAAGATTGAGGGTTCGAGTCC |
| | | | CTTCGTGGTCGNNN |
| 1250 | Arg | CCT | GCCCCAGTGGCCTAATGGATAAGGCACTGGC |
| | | | CTCCTAAGCCAGGGATTGTGGGTTCGAGTCC |
| | | | CACCTGGGGTANNN |
| 1251 | Arg | TCG | GACCGCGTGGCCTAATGGATAAGGCGTCTGA |
| | | | CTTCGGATCAGAAGATTGAGGGTTCGAGTCC |
| | | | CTCCGTGGTCGNNN |
| 1252 | Arg | TCT | GGCTCTGTGGCGCAATGGATNAGCGCATTGG |
| | | | ACTTCTAATTCAAAGGTTGCGGGTTCGAGTC |
| | | | CCNCCAGAGTCGNNN |
| 1253 | Asn | GTT | GTCTCTGTGGCGCAATCGGTTAGCGCGTTCG |
| | | | GCTGTTAACCGNAAAGGTTGGTGGTTCGAGC |
| | | | CCACCCAGGGACGNNN |
| 1254 | Asp | GTC | TCCTCGTTAGTATAGTGGTGAGTATCCCCGCC |
| | | | TGTCACGCGGGAGACCGGGGTTCGATTCCCC |
| | | | GACGGGGAGNNN |
| 1255 | Cys | GCA | GGGGGTATAGCTCAGNGGGTAGAGCATTTGA |
| | | | CTGCAGATCAAGAGGTCCCCGGTTCAAATCC |
| | | | GGGTGCCCCCTNNN |
| 1256 | Gln | CTG | GGTTCCATGGTGTAATGGTNAGCACTCTGGA |
| | | | CTCTGAATCCAGCGATCCGAGTTCAAGTCTC |
| | | | GGTGGAACCTNNN |
| 1257 | Gln | TTG | GGTCCCATGGTGTAATGGTTAGCACTCTGGA |
| | | | CTTTGAATCCAGCGATCCGAGTTCAAATCTC |
| | | | GGTGGGACCTNNN |
| 1258 | Glu | CTC | TCCCTGGTGGTCTAGTGGTTAGGATTCGGCG |
| | | | CTCTCACCGCCGCGGCCCGGGTTCGATTCCC |
| | | | GGTCAGGGAANNN |
| 1259 | Glu | TTC | TCCCTGGTGGTCTAGTGGCTAGGATTCGGCG |
| | | | CTTTCACCGCNGCGGCCCGGGTTCGATTCCC |
| | | | GGTCAGGGAANNN |
| 1260 | Gly | CCC | GCATTGGTGGTTCAGTGGTAGAATTCTCGCCT |
| | | | CCCACGCNGGAGACCCGGGTTCGATTCCCGG |
| | | | CCAATGCANNN |
| 1261 | Gly | GCC | GCATTGGTGGTTCAGTGGTAGAATTCTCGCCT |
| | | | GCCACGCGGGAGGCCCGGGTTCGATTCCCGG |
| | | | CCAATGCANNN |
| 1262 | Gly | TCC | GCGTTGGTGGTATAGTGGTGAGCATAGCTGC |
| | | | CTTCCAAGCAGTTGACCCGGGTTCGATTCCC |
| | | | GGCCAACGCANNN |
| 1263 | Ile | AAT | GGCCGGTTAGCTCAGTTGGTTAGAGCGTGGT |
| | | | GCTAATAACGCCAAGGTCGCGGGTTCGATCC |
| | | | CCGTACGGGCCANNN |
| 1264 | Ile | TAT | GCTCCAGTGGCGCAATCGGTTAGCGCGCGGT |
| | | | ACTTATAATGCCGAGGTTGTGAGTTCGAGCC |
| | | | TCACCTGGAGCANNN |
| 1265 | Leu | AAG | GGTAGCGTGGCCGAGCGGTCTAAGGCGCTGG |
| | | | ATTAAGGCTCCAGTCTCTTCGGGGGCGTGGG |
| | | | TTCGAATCCCACCGCTGCCANNN |
| 1266 | Leu | CAA | GTCAGGATGGCCGAGTGGTCNTAAGGCGCCA |
| | | | GACTCAAGTTCTGGTCTCCGNATGGAGGCGT |
| | | | GGGTTCGAATCCCACTTCTGACANNN |
| 1267 | Leu | CAG | GTCAGGATGGCCGAGCGGTCTAAGGCGCTGC |
| | | | GTTCAGGTCGCAGTCTCCCCTGGAGGCGTGG |
| | | | GTTCGAATCCCACTCCTGACANNN |
| 1268 | Leu | TAA | ACCAGGATGGCCGAGTGGTTAAGGCGTTGGA |
| | | | CTTAAGATCCAATGGACAGATGTCCGCGTGG |
| | | | GTTCGAACCCCACTCCTGGTANNN |
| 1269 | Leu | TAG | GGTAGCGTGGCCGAGCGGTCTAAGGCGCTGG |
| | | | ATTTAGGCTCCAGTCTCTTCGGNGGCGTGGG |
| | | | TTCGAATCCCACCGCTGCCANNN |
| 1270 | Lys | CTT | GCCCGGCTAGCTCAGTCGGTAGAGCATGAGA |
| | | | CTCTTAATCTCAGGGTCGTGGGTTCGAGCCCC |
| | | | ACGTTGGGCGNNNNNN |
| 1271 | Lys | TTT | GCCTGGATAGCTCAGTCGGTAGAGCATCAGA |
| | | | CTTTTAATCTGAGGGTCCAGGGTTCAAGTCCC |
| | | | TGTTCAGGCGNNN |
| 1272 | Met | CAT | GCCCTCTTAGCGCAGTNGGCAGCGCGTCAGT |
| | | | CTCATAATCTGAAGGTCCTGAGTTCGAGCCT |
| | | | CAGAGAGGGCANNN |
| 1273 | Phe | GAA | GCCGAAATAGCTCAGTTGGGAGAGCGTTAGA |
| | | | CTGAAGATCNTAAAGGTCCCTGGTTCAATCC |
| | | | CGGGTTTCGGCANNN |
| 1274 | Pro | AGG | GGCTCGTTGGTCTAGGGGTATGATTCTCGCTT |
| | | | AGGATGCGAGAGGTCCCGGGTTCAAATCCCG |
| | | | GACGAGCCCNNN |
| 1275 | Pro | CGG | GGCTCGTTGGTCTAGGGGTATGATTCTCGCTT |
| | | | CGGGTGCGAGAGGTCCCGGGTTCAAATCCCG |
| | | | GACGAGCCCNNN |
| 1276 | Pro | TGG | GGCTCGTTGGTCTAGGGGTATGATTCTCGCTT |
| | | | TGGGTGCGAGAGGTCCCGGGTTCAAATCCCG |
| | | | GACGAGCCCNNN |
| 1277 | Ser | AGA | GTAGTCGTGGCCGAGTGGTTAAGGCGATGGA |
| | | | CTAGAAATCCATTGGGGTTTCCCCGCGCAGG |
| | | | TTCGAATCCTGCCGACTACGNNN |
| 1278 | Ser | CGA | GCTGTGATGGCCGAGTGGTTAAGGCGTTGGA |
| | | | CTCGAAATCCAATGGGGTCTCCCCGCGCAGG |
| | | | TTCGAATCCTGCTCACAGCGNNN |
| 1279 | Ser | GCT | GACGAGGNNTGGCCGAGTGGTTAAGGCGAT |
| | | | GGACTGCTAATCCATTGTGCTCTGCACGCGT |
| | | | GGGTTCGAATCCCATCCTCGTCGNNN |
| 1280 | Ser | TGA | GTAGTCGTGGCCGAGTGGTTAAGGCGATGGA |
| | | | CTTGAAATCCATTGGGGTCTCCCCGCGCAGG |
| | | | TTCGAATCCTGCCGGCTACGNNN |
| 1281 | Thr | AGT | GGCTCCGTGGCTTAGCTGGTTAAAGCGCCTG |
| | | | TCTAGTAAACAGGAGATCCTGGGTTCGAATC |
| | | | CCAGCGGGGCCTNNN |
| 1282 | Thr | CGT | GGCNCTGTGGCTNAGTNGGNTAAAGCGCCGG |
| | | | TCTCGTAAACCNGGAGATCNTGGGTTCGAAT |
| | | | CCCANCNGGGCCTNNN |
| 1283 | Thr | TGT | GGCTCCATAGCTCAGNGGGTTAGAGCACTGG |
| | | | TCTTGTAAACCAGGGGTCGCGAGTTCAAATC |
| | | | TCGCTGGGGCCTNNN |
| 1284 | Trp | CCA | GACCTCGTGGCGCAACGGTAGCGCGTCTGAC |
| | | | TCCAGATCAGAAGGTTGCGTGTTCAAATCAC |
| | | | GTCGGGGTCANNN |
| 1285 | Tyr | GTA | CCTTCGATAGCTCAGCTGGTAGAGCGGAGGA |
| | | | CTGTAGATCCTTAGGTCGCTGGTTCGATTCCG |
| | | | GCTCGAAGGANNN |
| 1286 | Val | AAC | GTTTCCGTAGTGTAGTGGTTATCACGTTCGCC |
| | | | TAACACGCGAAAGGTCCCCGGTTCGAAACCG |
| | | | GGCGGAAACANNN |
| 1287 | Val | CAC | GTTTCCGTAGTGTAGTGGTTATCACGTTCGCC |
| | | | TCACACGCGAAAGGTCCCCGGTTCGAAACCG |
| | | | GGCGGAAACANNN |
| 1288 | Val | TAC | GGTTCCATAGTGTAGTGGTTATCACGTCTGCT |
| | | | TTACACGCAGAAGGTCCTGGGTTCGAGCCCC |
| | | | AGTGGAACCANNN |
| 1289 | iMet | CAT | AGCAGAGTGGCGCAGCGGAAGCGTGCTGGG |
| | | | CCCATAACCCAGAGGTCGATGGATCGAAACC |
| | | | ATCCTCTGCTANNN |

TABLE 11
Score values alignment

| | Candidate | Reference | Match |
|---|---|---|---|
| Row | nucleotide | nucleotide | score |
| 1 | A | A | 1 |
| 2 | T | T | 1 |
| 3 | U | T | 1 |
| 4 | C | C | 1 |
| 5 | G | G | 1 |
| 6 | A | N | 0 |
| 7 | T | N | 0 |
| 8 | C | N | 0 |
| 9 | G | N | 0 |
| 10 | N | A | 0 |
| 11 | N | T | 0 |
| 12 | N | C | 0 |
| 13 | N | G | 0 |
| 14 | N | N | 0 |

### Premature Termination Codons (PTC) and ORFs Comprising PTCs

Mutations underlie many diseases. For example, a point mutation in the open reading frame (ORF) of a gene which creates a premature stop codon (PTC) can result in altered expression and/or activity of a polypeptide encoded by the gene. Table 15 provides single mutations in codons encoding amino acids which can result in a stop codon. In an embodiment, a PTC disclosed herein comprises a mutation disclosed in Table 15.

In an embodiment, the codon having the first sequence or the PTC comprises a mutation disclosed in Table 15. In an embodiment, the non-mutated, e.g., wildtype, codon sequence of the codon having the first sequence or the PTC is an original codon sequence provided in Table 15 and the amino acid corresponding to the non-mutated codon is an original AA provided in Table 15.

In an embodiment, the TREM, TREM core fragment or TREM fragment recognizes a stop codon and mediates incorporation of the original AA provided in Table 15 at the position of the stop codon. In an embodiment, the TREM, TREM core fragment or TREM fragment recognizes a stop codon and mediates incorporation of an amino acid belonging to the same group as the original AA, e.g., as provided in Table 16. Other genetic abnormalities, such as insertions and/or deletions can also result in a PTC in an ORF.

TABLE 15
Select amino acids and stop codons

| Original | Original | One mutation |
|---|---|---|
| AA | codon | to stop codon |
| TRP | UGG | UGA |
| TYR | UAU | UAA |
| | UAC | UAG |
| CYS | UGU | UGA |
| | UGC | UGA |
| GLU | GAA | UAA |
| | GAG | UAG |
| LYS | AAA | UAA |
| | AAG | UAG |
| GLN | CAA | UAA |
| | CAG | UAG |
| SER | UCA | UGA |
| | UCG | UAG |
| LEU | UUA | UAA OR UGA |
| | UUG | UAG |
| ARG | CGA | UGA |
| GLY | GGA | UGA |

TABLE 16
Amino acids and amino acid groupings

| Group | Amino acid |
|---|---|
| Nonpolar, aliphatic R group | leucine |
| | methionine |
| | isoleucine |
| | glycine |
| | alanine |
| | valine |
| Polar, uncharged R group | serine |
| | threonine |
| | cysteine |
| | proline |
| | asparagine |
| | glutamine |
| positively charged r group | lysine |
| | arginine |
| | histidine |
| Negatively charged R group | aspartate |
| | glutamate |
| Nonpolar, aromatic R group | phenylalanine |
| | tyrosine |
| | tryptophan |

Disclosed herein, inter alia, are endogenous ORFs comprising a codon having a first sequence. e.g., a mutation. e.g., a PTC. An ORF having a PTC, e.g., as described herein, can be present, or part of in any gene. As an example, the ORF can be present or be part of any gene in the human genome.

In an embodiment, a PTC disclosed herein is present in a gene disclosed in any one of Tables 17, 18, or 20. Exemplary genes having ORFs comprising a PTC are provided in Table 3.

TABLE 17
Exemplary genes with ORFs having a PTC

| | | | | |
|---|---|---|---|---|
| A2ML1 | ARFGEF1 | CACNA1G | CNOT1 | DLG4 |
| AARS1 | ARFGEF2 | CACNA1S | COG1 | DLL1 |
| AARS2 | ARHGAP21 | CACNA2D1 | COL11A2 | DNA2 |
| ABCA13 | ARHGEF9 | CACNA2D2 | COL13A1 | DNM1L |
| ABCB11 | ARMC4 | CACNB2, NSUN6 | COL4A1 | DNMT1 |
| ABCG5 | ARV1 | CAD | COL4A2 | DNMT3A |
| ABHD5 | ARX | CAMTA1 | COL4A4 | DNMT3B |
| ACAD8 | ASCC3 | CARS2 | COL9A1 | DPH1 |
| ACADL | ASH1L | CCDC140 | COQ4 | DPYD-AS1 |
| ACSF3 | ASPH | CCDC8 | COQ6 | DSEL |
| ACTA2 | ASXL2 | CCM2 | COX14 | DSPP |
| ACTC1, | ATAD3A | CD40LG | CPE | DUOXA1 |
| ACTN2 | ATP2A2 | CDAN1 | CPEB1-AS1 | DUOXA2 |
| ACVR1 | ATP6V1B1 | CDH15 | CREBBP | DVL1 |
| ADAR | ATP8A2 | CDK11A | CRELD1 | EARS2 |
| ADAT3 | AUH | CEBPA | CSNK2A1 | EBF3 |
| ADCY5 | AUTS2 | CELF5 | CSNK2B | EBP |
| ADIPOQ | AVPR2 | CELSR2 | CSTA | EDAR |
| ADIPOQ-AS1 | B3GLCT | CEP135 | CTNND2 | EFHC1 |
| ADIPOR1 | B4GAT1 | CEP164 | CTSA | EFNB1 |
| AFF2 | BCAP31 | CEP83 | CTSC | EFTUD2 |
| ALG11 | BCL11A | CETP | CUL3 | EIF2B5 |
| ALG14 | BCL11B | CFI | CYLD | ELANE |
| ALG6 | BCORL1 | CHAMP1 | CYP11A1 | EMC1 |
| ALOXE3 | BEND2 | CHAT | DAG1 | EMC1-AS1 |
| AMER1 | BGN | CHD1 | DARS2 | ENO3 |
| AMH | BMP4 | CHD4 | DCX | ENTPD5 |
| AMMECR1 | BRD4 | CHD8 | DDHD2 | EP300 |
| AMN | BRPF1 | CHRM2 | DDR2 | EPM2A |
| ANK2 | BRSK2 | CHRNB1 | DEAF1 | ERLIN2 |
| ANK3 | BUB1B | CIC | DENND5A | EVC |
| ANKS6 | BUB1B-PAK6 | CLCN7 | DGAT1 | EZH2 |
| ANOS1 | C8G | CLTC | DHFR | FAM111A |
| AP1S1 | CACNA1E | CNGA3 | DIAPH3 | FAM126A |
| AP3B2 | CACNA1F | CNKSR2 | DISP1 | FAN1 |
| FANCD2 | GJB6 | ISLR2 | LOC110673972 | MTR |
| FANCE | GJC2 | ITGA3 | LOC112997540 | MYCBP2 |
| FASTKD2 | GK | ITGA8 | LOC113788297 | MYCNOS |
| FAT1 | GLI2 | ITGB6 | LOC349160 | MYH7B |
| FBN2 | GNAI1 | JMJD1C | LONP1 | MYL3 |
| FBP1 | GNB1L | KANK1 | LORICRIN | MYOM1 |
| FDXR | GNE | KANSL1 | LPIN1 | MYPN |
| FGA | GNRH1 | KBTBD13 | LPIN2 | MYT1L |
| FGD1 | GNS | KCNA2 | LRP2 | NAA15 |
| FGF10 | GPAA1 | KCNB1 | LRRTM4 | NAGA |
| FGFR1 | GPD1L | KCND2 | MAB21L2 | NARS2 |
| FGFR2 | GRIN2A | KCNE3 | MAF | NAXE |
| FIBP | GTPBP3 | KCNMA1 | MARS1 | NCAPH2 |
| FLAD1 | HACE1 | KCNQ5 | MARS2 | NCF2 |
| FLG-AS1 | HADH | KCTD7 | MASP1 | NDP |
| FLVCR1 | HADHB | KIDINS220 | MBOAT7 | NDP-AS1 |
| FLVCR2 | HDAC4 | KIF21B | MCM3AP | NDRG1 |
| FMN2 | HERC1 | KIF6 | MCM3AP-AS1 | NDUFA2, TMCO6 |
| FOXA2 | HESX1 | KIT | MED13 | NDUFAF1 |
| FOXC1 | HIBCH | KLHL40 | MED17 | NDUFAF5 |
| FOXC2 | HNF4A | KLHL41 | MEGF10 | NDUFAF6 |
| FOXC2-AS1 | HNRNPH2 | KNL1 | MET | NDUFAF7 |
| FOXP2 | HPRT1 | KRAS | MGAT2 | NDUFS1 |
| FREM1 | HRG | LAMB1 | MIB1 | NDUFS3 |
| FRYL | HUWE1 | LAMB2 | MICU1 | NEFH |
| FTL | IARS1 | LAMC3 | MIR302CHG | NEK8 |
| FUS | IBA57 | LARP7 | MIR5004 | NEXMIF |
| GABRG2 | IDH2 | LARS1 | MIR6501 | NFIA |
| GAN | IFNAR1 | LCT | MITD1 | NFKB1 |
| GATA2 | IFT122 | LEMD3 | MMP13 | NHEJ1 |
| GATA4 | IFT80 | LGI4 | MMP21 | NICN1 |
| GATAD1 | IGF2 | LIAS | MNX1 | NID1 |
| GDF5 | ILDR1 | LINS1 | MNX1-AS2 | NKX2-1 |
| GDF5-AS1 | ILK, TAF10 | LIPC | MPDU1 | NLRP1 |
| GFM1 | INF2 | LIPT1 | MRPS22 | NLRP3 |
| GH-LCR | INS-IGF2 | LOC101448202 | MSL3 | NOD2 |
| GHRHR | INSR | LOC106804612, HBA2 | MSRB3 | NONO |
| GHSR | IRAK1BP1 | LOC107303338 | MT-ND2 | NOTCH3 |
| GJA1 | IRAK3 | RARS2 | SETD1B | SPTLC1 |
| NPHP4 | PLEKHG5 | RAX | SETD2 | SRD5A3 |
| NPR2 | PLEKHM2 | RBM10 | SETX | SRPX2 |
| NR2F2 | PLK4 | RELN | SFTA3 | SSBP2 |
| NR5A1 | PLPBP | RERE | SHANK2 | ST3GAL3 |
| NRL, PCK2 | PNKD | RFT1 | SHH | ST3GAL5 |
| NRXN1 | PNPLA1 | RMND1 | SIN3A | STAMBP |
| NT5DC1 | POC1A | RNASEH1 | SIX3 | STAT3 |
| NTRK2 | POLG2, MILR1 | RNF17 | SKI | STIL |
| NUBPL | POMGNT2 | ROR2 | SLC13A5 | STX11 |
| NUS1 | PPM1D | RP2 | SLC16A1 | STX1B |
| OCRL | PPP3CA | RPL11 | SLC16A2 | SUCLA2 |
| OPTN | PRDM1 | RPS19 | SLC17A8 | SYN1 |
| P4HA1 | PRDM12 | RRM2B | SLC18A3 | SYN2 |
| PAK6 | PREPL | RS1 | SLC20A2 | SYNJ1 |
| PBX1 | PRICKLE1 | RUNX2 | SLC25A4 | TAB2 |
| PCARE | PRKAG2 | RXYLT1 | SLC25A46 | TACR3 |
| PCDH12 | PROS1 | S100PBP | SLC2A1 | TBCD |
| PDCD10 | PRPF31 | SALL4 | SLC39A8 | TBL1XR1 |
| PDE11A-AS1 | PRPF8 | SAMD9 | SLC52A2 | TBX1 |
| PDE4D | PRPS1 | SAR1B | SLC6A3 | TBX18 |
| PDE6A | PRSS1, TRB | SASH3 | SLC6A8 | TCIRG1 |
| PDHX | PSAT1 | SBF2 | SLC6A9 | TELO2 |
| PDLIM3 | PSMD12 | SBF2-AS1 | SLC7A9 | TFAP2A |
| PDP1 | PSTPIP1 | SCAMP4 | SMAD2 | TFG |
| PDSS1 | PTCHD1 | SCLT1 | SMAD9 | TGIF1 |
| PEX5 | PTF1A | SCN10A | SMARCA2 | THAP1 |
| PHF21A | PTPN23 | SCN11A | SMC3 | TINF2 |
| PHF8 | PTPRQ | SCN1B | SMOC2 | TLK2 |
| PHKA2 | PTRH2 | SCN3A | SNTA1 | TMEM43 |
| PHKB | PUS1 | SCN4A | SNX14 | TMIE |
| PIEZO1 | PYCR2 | SCN4B | SNX22 | TMPO |
| PIGG | QARS1 | SCN8A | SOCS1 | TMPRSS15 |
| PIGL | RAB3GAP1, ZRANB3 | SCO2 | SOX11 | TMTC3 |
| PIGM | RAB3GAP2 | SCYL1 | SOX17 | TNFAIP3 |
| PIGP | RAC1 | SEMA4A | SPATA5 | TNFRSF11A |
| PIK3CA | RAF1 | SEPTIN9 | SPATA7 | TOE1 |
| PIN4, ERCC6L | RAG1 | SET | SPRED1 | TOR1AIP1 |
| PLAT | RARB | SETD1A | SPTBN2 | TPK1 |
| TPM1 | UTP14C | ZEB1 | | |
| TRAPPC9 | VPS53 | ZFHX4 | | |
| TRIM37 | WDR19 | ZFPM2 | | |
| TRIM59-IFT80 | WDR26 | ZFPM2-AS1 | | |
| TRIO | WDR62 | ZIC1 | | |
| TRIP12 | WDR81 | ZIC2 | | |
| TRIP4 | WNT1 | ZMYND11 | | |
| TRPM1 | WNT10A | ZNF335 | | |
| TSEN54 | WRAP53 | ZNF423 | | |
| TUBA4A | WWOX | ZNF469 | | |
| TUBGCP4 | YARS1 | | | |
| TUBGCP6 | YARS2 | | | |
| TWNK | YY1 | | | |
| TXNRD2 | ZAP70 | | | |
| UBA2 | ZBTB20 | | | |
| UBA5 | ZBTB24 | | | |
| UMOD | ZC4H2 | | | |
| UNC5B | ZDHHC9 | | | |

Additional exemplary genes containing a PTC include FVIII, FIX, CFTR, MeCP2, NAGLU, DMD, GAA, RP1, RP2, ABCA4, PCDH15, REP1, GLA, MUT, TP53, and ATM. In an embodiment, the PTC is present within the FVIII gene and comprises an R mutation, e.g., an R2228X mutation. In an embodiment, the PTC is present within the FIX gene and comprises an R mutation, e.g., an R29X mutation, an R116X mutation, an R248X mutation, an R252X mutation, an R333X mutation, and/or an R338X mutation. In an embodiment, the PTC is present within the CFTR gene and comprises an R mutation, e.g., an R553X mutation. In an embodiment, the PTC is present within the MeCP2 gene and comprises an R mutation, e.g., an R168X mutation. In an embodiment, the PTC is present within the NAGLU gene and comprises an R mutation, e.g., an R626X mutation. In an embodiment, t the PTC is present within the DMD gene and comprises an R mutation, e.g., an R3881X mutation. In an embodiment, the PTC is present within the GAA gene and comprises an R mutation, e.g., an R854X mutation. In an embodiment, the PTC is present within the RP1 gene and comprises an R mutation, e.g., an R667X mutation. In an embodiment, the PTC is present within the RP₂ gene and comprises an R mutation, e.g., an R120X mutation. In an embodiment, the PTC is present within the ABCA₄ gene and comprises an R mutation, e.g., an R2030X mutation. In an embodiment, the PTC is present within the PCD gene and comprises an R mutation, e.g., an R245X mutation. In an embodiment, the PTC is present within the REP1 gene and comprises an R mutation, e.g., an R270X mutation. In an embodiment, the PTC is a mutation in the GLA gene, e.g., an R220X mutation and/or an R227X mutation. In an embodiment, the PTC is present within the MUT gene and comprises an R mutation, e.g., an R228X mutation, an R403X mutation, an R467X mutation, and/or an R727X mutation. In an embodiment, the PTC is present within the TP53 gene and comprises an R mutation, e.g., an R578X mutation. In an embodiment the PTC is present within the ATM gene and comprises an R mutation, e.g., an R35X mutation.

Diseases or Disorders Associated with a PTC

A TREM composition disclosed herein can be used treat a disorder or disease associated with a PTC, e.g., as described herein. Exemplary diseases or disorders associated with a PTC are listed in Tables 18, 19, and 20.

In an embodiment, the subject has a disease or disorder provided in any one of Tables 4-6. In an embodiment, the cell is associated with, e.g., is obtained from a subject who has, a disorder or disease listed in any one of Tables 18-20.

For example, the disorder or disease can be chosen from the left column of Table 18. As another example, the disorder or disease is chosen from the left column of Table 18 and, in embodiments the PTC is in a gene chosen from the right column of Table 18, e.g., any one of the genes provided in the right column of Table 18. In some embodiments, the PTC is in a gene corresponding to the disorder or disease provided in the left column of Table 18. As a further non-limiting example, the PTC can be at a position provided in Table 18.

As another example, the disorder or symptom is chosen from a disorder or disease provided in Table 19.

As yet another exmaple, the disorder or symptom is chosen from a disorder or disease provided in Table 20. In an embodiment, the disorder or symptom is chosen from a disorder or disease provided in Table 20 and, in embodiments, the PTC is in any gene provided in Table 20. In an embodiment, the disorder or symptom is chosen from a disorder or disease provided in Table 20 and the PTC is in a corresponding gene provided in Table 20, e.g., a gene corresponding to the disease or disorder. In an embodiment, the disorder or symptom is chosen from a disorder or disease provided in Table 20 and the PTC is not in a gene provided in Table 20.

In an embodiment of any of the methods disclosed herein, the PTC is at any position within the ORF of the gene. e.g., upstream of the naturally occurring stop codon.

TABLE 18
Exemplary diseases or disorders

| | |
|---|---|
| Disease/disorder or protein | Exemplary Point Mutation |

G to A point mutations

| | |
|---|---|
| Dihydropyrimidine dehydrogenase | NM 000110.3(DPYD): c.1905 + 1G > A |
| deficiency | |
| Noonan syndrome | NM 005633.3(SOS1): c.2536G > A |
| | (p.Glu846Lys) |
| Lynch syndrome | NM 000251.2(MSH2): c.212 − 1G > A |
| Breast-ovarian cancer, familial 1 | NM 007294.3(BRCA1): c.963G > A |
| | (p.Trp321Ter) |
| Cystic fibrosis | NM 000492.3(CFTR): c.57G > A (p.Trpl9Ter) |
| Anemia, due to G6PD deficiency | NM 000402.4(G6PD): c.292G > A |
| | (p.Val98Met) |
| AVPR2 | NM 000054.4(AVPR2): c.878G > A |
| Nephrogenic diabetes insipidus, X-linked | (p.Trp293Ter) |
| FANCC | NM 000054.4(AVPR2): c.878G > A |
| Fanconi anemia, complementation group C | (p.Trp293Ter) |
| FANCC | NM 000136.2(FANCC): c.1517G > A |
| Fanconi anemia, complementation group C | (p.Trp506Ter) |
| IL2RG | NM 000206.2(IL2RG): c.710G > A |
| X-linked severe combined | (p.Trp237Ter) |
| immunodeficiency | |
| F8 Hereditary factor VIII deficiency | NM 000132.3(F8): c.3144G > A |
| disease | (p.Trpl048Ter) |
| LDLR | NM 000527.4(LDLR): c.1449G > A |
| Familial hypercholesterolemia | (p.Trp483Ter) |
| CBS | NM 000071.2(CBS): c.162G > A |
| Homocystinuria due to CBS deficiency | (p.Trp54Ter) |
| HBB | NM 000518.4(HBB): c. 114G > A |
| betaThalassemia | (p.Trp38Ter) |
| ALDOB | NM 000035.3(ALDOB): c.888G > A |
| Hereditary fmctosuria | (p.Trp296Ter) |
| DMD | NM 004006.2(DMD): c.3747G > A |
| Duchenne muscular dystrophy | (p.Trpl249Ter) |
| SMAD4 | NM 005359.5(SMAD4): c.906G > A |
| Juvenile polyposis syndrome | (p.Trp302Ter) |
| BRCA2 | NM 000059.3(BRCA2): c.582G > A |
| Familial cancer ofbreastlBreast-ovarian | (p.Trpl94Ter) |
| cancer, familial 2 | |
| GRIN2A | NM 000833.4(GRIN2A): c.3813G > A |
| Epilepsy, focal, with speech disorder and | (p.Trpl271Ter) |
| with or without mental retardation | |
| SCN9A | NM 002977.3(SCN9A): c.2691G > A |
| Indifference to pain, congenital, | (p.Trp897Ter) |
| autosomal recessive | |
| TARDBP | NM 007375.3(TARDBP): c.943G > A |
| Amyotrophic lateral sclerosis type 10 | (p.Ala315Thr) |
| CFTR | NM 000492.3(CFTR): c.3846G > A |
| Cystic fibrosislHereditary | (p.Trpl282Ter) |
| pancreatitislnot providedlataluren | |
| response - Efficacy | |
| UBE3A | NM 130838. l(UBE3A): c.2304G > A |
| Angelman syndrome | (p.Trp768Ter) |
| SMPD1 | NM 000543.4(SMPD1): c.168G > A |
| Niemann-Pick disease, type A | (p.Trp56Ter) |
| USH2A | NM 206933.2(USH2A): c.9390G > A |
| Usher syndrome, type 2A | (p.Trp3130Ter) |
| MENl | NM 130799.2(MEN1): c.1269G > A |
| Hereditary cancer-predisposing syndrome | (p.Trp423Ter) |
| C8orf37 | NM 177965.3(C8orf37): c.555G > A |
| Retinitis pigmentosa 64 | (p.Trpl85Ter) |
| MLHl | NM 000249.3(MLH1): c.1998G > A |
| Lynch syndrome | (p.Trp666Ter) |
| TSC2 | NM 000548.4(TSC2): c.2108G > A |
| Tuberous sclerosis 21Tuberous | (p.Trp703Ter) |
| sclerosis syndrome 46 | |
| NFl | NM 000267.3(NF1): c.7044G > A |
| Neurofibromatosis, type 1 | (p.Trp2348Ter) |
| MSH6 | NM 000179.2(MSH6): c.3020G > A |
| Lynch syndrome | (p.Trpl007Ter) |
| SMNl | NM 000344.3(SMN1): c.305G > A |
| Spinal muscular atrophy, type III | (p.Trpl02Ter) |
| Kugelberg- Welander disease | |
| SH3TC2 | NM 024577.3(SH3TC2): c.920G > A |
| Charcot-Marie-Tooth disease, type 4C | (p.Trp307Ter) |
| DNAH5 | NM 001369.2(DNAH5): c.8465G > A |
| Primary ciliary dyskinesia | (p.Trp2822Ter) |
| MECP2 | NM 004992.3(MECP2): c.311G > A |
| Rett syndrome | (p.Trpl04Ter) |
| ADGRVl | NM 032119.3(ADGRV1): c.7406G > A |
| Usher syndrome, type 2C | (p.Trp2469Ter) |
| AHil | NM 017651.4(AHI1): c.2174G > A |
| Joubert syndrome 3 | (p.Trp725Ter) |
| PRKN | NM 004562.2(PRKN): c.1358G > A |
| Parkinson disease 2 | (p.Trp453Ter) |
| COL3Al | NM 000090.3(COL3Al): c.3833G > A |
| Ehlers-Danlos syndrome, type 4 | (p.Trpl278Ter) |
| BRCAl | NM 007294.3(BRCA1): c.5511G > A |
| Familial cancer ofbreastlBreast-ovarian | (p.Trpl837Ter) |
| cancer, familial 1 | |
| MYBPC3 | NM 000256.3(MYBPC3): c.3293G > A |
| Primary familial hypertrophic | (p.Trpl098Ter) |
| cardiomyopathy | |
| APC | NM 000038.5(APC): c.1262G > A |
| Familial adenomatous polyposis 1 | (p.Trp421Ter) |
| BMPR2 | NM 001204.6(BMPR2): c.893G > A |
| Primary pulmonary hypertension | (p.W298*) |

T to C point mutations

| | |
|---|---|
| Wilson disease | NM_000053.3(ATP7B): c.3443T > C |
| | (p.Ile l l 48Thr) |
| Leukodystrophy, hypomyelinating, 2 | NM_020435.3(GJC2): c.857T > C |
| | (p.Met286Thr) |
| Alport syndrome, X-linked recessive | NM_000495.4(COL4A5): c.438 + 2T > C |
| Leigh disease | NC 012920.l: m.9478T > C |
| Gaucher disease, type 1 | NM_001005741.2(GBA): c.751T > C |
| | (p.Tyr251His) |
| Renal dysplasia, retinal pigmentary | NM_0l4714.3(IFT140): c.4078T > C |
| dystrophy, cerebellar ataxia and skeletal | (p.Cysl360Arg) |
| dysplasia | |
| Marfan syndrome | NM_000138.4(FBN1): c.3793T > C |
| | (p.Cysl265Arg) |
| Deficiency of UDPglucose-hexose-1- | NM_000155.3(GALT): c.482T > C |
| phosphate uridylyltransferase | (p.Leul61Pro) |
| Familial hypercholesterolemia | NM_000527.4(LDLR): c.694 + 2T > C |
| Episodic pain syndrome, familial, 3 | NM_001287223.1(SCN11A): c.1142T > C |
| | (p.Ile381Thr) |
| Navajo neurohepatopathy | NM_002437.4(MPV17): c.186 + 2T > C |
| Congenital muscular dystrophy, LMNA- | NM_l 70707.3(LMNA): c.l139T > C |
| related | (p.Leu380Ser) |
| Hereditary factor VIII deficiency disease | NM_000132.3(F8): c.5372T > C (p.Metl |
| | 791Thr) |
| Insulin-dependent diabetes mellitus | NM_0l4009.3(FOXP3): c.970T > C |
| secretory diarrhea syndrome | (p.Phe324Leu) |
| Hereditary factor IX deficiency disease | NM_000133.3(F9): c.1328T > C (p.Ile443Thr) |
| Familial cancer of breast, Breast-ovarian | NM_000059.3(BRCA2): c.316 + 2T > C |
| cancer, familial 2, Hereditary cancer | |
| predisposing syndrome | |
| Cardiac arrhythmia | NM_000238.3(KCNH2): c.1945 + 6T > C |
| Tangier disease | NM_005502.3(ABCA1): c.4429T > C |
| | (p.Cysl477Arg) |
| Dilated cardiomyopathy lAA | NM_001103.3(ACTN2): c.683T > C |
| | (p.Met228Thr) |
| Mental retardation 3, X-linked | NM_005334.2(HCFC1): c.−970T > C |
| Limb-girdle muscular dystrophy, type 2B | NM_003494.3(DYSF): c.1284 + 2T > C |
| Macular dystrophy, vitelliform, 5 | NM_0l6247.3(IMPG2): c.370T > C |
| | (p.Phel24Leu) |
| Retinitis pigmentosa | NM_000322.4(PRPH2): c.736T > C |
| | (p.Trp246Arg) |

TABLE 19
Additional exemplary disorders

| | |
|---|---|
| 5q-syndrome | Adams-Oliver syndrome 1 |
| Adams-Oliver syndrome 3 | Adams-Oliver syndrome 5 |
| Adams-Oliver syndrome 6 | Alagille syndrome 1 |
| Autoimmune lymphoproliferative syndrome | Autoimmune lymphoproliferative syndrome |
| type IA | type V |
| Autosomal dominant deafness-2A | Brain malformations with or without urinary |
| | tract defects (BRMUTD) |
| Carney complex type 1 | CHARGE syndrome |
| Cleidocranial dysplasia | Currarino syndrome |
| Denys-Drash syndrome/Frasier syndrome | Developmental delay |
| intellectual disability | obesity |
| and dysmorphic features (DIDOD) | DiGeorge syndrome (TBXl-associated) |
| Dravet syndrome | Duane-radial ray syndrome |
| Ehlers-Danlos syndrome (classic-like) | Ehlers-Danlos syndrome (vascular type) |
| Feingold syndrome 1 | Frontotemporal lobar degeneration with |
| | TDP43 inclusions (FTFD-TDP) |
| GRN-related | GFUT1 deficiency syndrome |
| Greig cephalopolysyndactyly syndrome | Hereditary hemorrhagic telangiectasia type 1 |
| Holoprosencephaly 3 | Holoprosencephaly 4 |
| Holoprosencephaly 5 | Holt-Oram syndrome |
| Hypoparathyroidism | sensorineural deafness |
| and renal disease (HDR) | Kleefstra syndrome 1 |
| Klippel-Trenaunay syndrome (AAGF-related) | Feri-Weill dyschondrosteosis |
| Marfan syndrome | Mental retardation and distinctive facial |
| | features with or without cardiac defects |
| | (MRFACD) |
| Mental retardation | autosomal dominant 1 |
| Mental retardation | autosomal dominant 19 |
| Mental retardation | autosomal dominant 29 |
| Nail-patella syndrome (NPS) | Phelan-McDermid syndrome |
| Pitt-Hopkins syndrome | Primary pulmonary hypertension 1 |
| Rett syndrome (congenital variant) | Smith-Magenis syndrome (RAI1-associated) |
| Sotos syndrome 1 | Sotos syndrome 2 |
| Stickler syndrome type I | Supravalvular aortic stenosis |
| SYNGAP1 -related intellectual disability | Treacher Collins syndrome |
| Trichorhinophalangeal syndrome type I | Ulnar-mammary syndrome |
| van der Woude syndrome 1 | Waardenburg syndrome type 1 |
| Waardenburg syndrome type 2A | Waardenburg syndrome type 4C. |

TABLE 20
Exemplary genes with ORFs comprising a PTC and exemplary disorders

| Gene | Disease/Disorder |
|---|---|
| AAAS | Glucocorticoid deficiency with achalasia |
| AAGAB | Keratosis palmoplantaris papulosa |
| AASS | Hyperlysinemia |
| ABCA1 | Tangier disease |
| ABCA12, | Autosomal recessive congenital ichthyosis 4B |
| SNHG31 | |
| ABCA3 | 3, Surfactant metabolism dysfunction, pulmonary |
| ABCA4 | Bietti crystalline corneoretinal dystrophy, Cone-rod degeneration, Cone-rod |
| | dystrophy 3, Macular dystrophy, Retinal dystrophy, Retinitis pigmentosa, Retinitis |
| | pigmentosa 19, Stargardt disease, Stargardt disease 1 |
| ABCB4 | Cholestasis, Progressive familial intrahepatic cholestasis 3, intrahepatic, of |
| | pregnancy 3 |
| ABCC2 | Dubin-Johnson syndrome |
| ABCC6 | Cutis laxa, Generalized arterial calcification of infancy 2, Papule, Pseudoxanthoma |
| | elasticum, forme firuste |
| ABCC8 | 1, Familial hyperinsulinism, Hyperinsulinemic hypoglycemia, familial |
| ABCC9 | Arrhythmogenic right ventricular cardiomyopathy, Cardiomyopathy, |
| | Cardiovascular phenotype, Dilated cardiomyopathy 1O, Primary dilated |
| | cardiomyopathy |
| ABCD1 | Adrenoleukodystrophy, Spastic gait, Spastic paraplegia |
| ABHD12 | Polyneuropathy, and cataract, ataxia, hearing loss, retinitis pigmentosa |
| ABRAXAS1 | Hereditary breast and ovarian cancer syndrome |
| ACAD9 | Acyl-CoA dehydrogenase family, deficiency of, member 9 |
| ACADM | Medium-chain acyl-coenzyme A dehydrogenase deficiency |
| ACADS | Deficiency of butyryl-CoA dehydrogenase |
| ACADVL | Very long chain acyl-CoA dehydrogenase deficiency |
| ACAN | Osteochondritis dissecans, Spondyloepiphyseal dysplasia, kimberley type |
| ACAT1 | Deficiency of acetyl-CoA acetyltransferase |
| ACBD5 | RETINAL DYSTROPHY WITH LEUKODYSTROPHY |
| ACBD6, LHX4, | Short stature-pituitary and cerebellar defects-small sella turcica syndrome |
| LHX4-AS1 | |
| ACE | Renal dysplasia |
| ACOX1 | Peroxisomal acyl-CoA oxidase deficiency |
| ACP5 | Spondyloenchondrodysplasia with immune dysregulation |
| ACP5, ZNF627 | Spondyloenchondrodysplasia with immune dysregulation |
| ACTA1 | Congenital myopathy with excess of thin filaments |
| ACTB | Baraitser-Winter syndrome |
| ACVRL1 | Hereditary hemorrhagic telangiectasia type 1, Primary pulmonary hypertension, |
| | Pulmonary arterial hypertension related to hereditary hemorrhagic telangiectasia, |
| | Telangiectasia, hereditary hemorrhagic, type 2 |
| ACY1 | Neurological conditions associated with aminoacylase 1 deficiency |
| ADA | Severe combined immunodeficiency disease, Severe combined immunodeficiency |
| | due to ADA deficiency |
| ADAM10 | Reticulate acropigmentation of Kitamura |
| ADAMTS17 | Weill-Marchesani syndrome 4 |
| ADAMTS2 | Ehlers-Danlos syndrome dermatosparaxis type |
| ADAMTSL4 | Ectopia lentis et pupillae |
| ADAMTSL4 | Ectopia lentis, Ectopia lentis 2, Ectopia lentis et pupillae, autosomal recessive, |
| | isolated |
| ADCY3 | BODY MASS INDEX QUANTITATIVE TRAIT LOCUS 19 |
| ADCY3, CENPO | BODY MASS INDEX QUANTITATIVE TRAIT LOCUS 19 |
| ADGRG1 | Polymicrogyria, bilateral frontoparietal |
| ADGRG2 | Congenital bilateral aplasia of vas deferens from CFTR mutation, Vas deferens, X- |
| | linked, congenital bilateral aplasia of |
| ADGRG6 | Arthrogryposis multiplex congenita, Lethal congenital contracture syndrome 9 |
| ADGRV1 | 4, Febrile seizures, Rare genetic deafness, Retinal dystrophy, Usher syndrome, |
| | familial, type 2C |
| ADNP | Helsmoortel-Van der Aa Syndrome, History of neurodevelopmental disorder, |
| | Inborn genetic diseases |
| AEBP1 | 2, CLASSIC-LIKE, EHLERS-DANLOS SYNDROME |
| AGA | Aspartylglucosaminuria |
| AGK | Sengers syndrome |
| AGK, DENND11 | Cataract, Sengers syndrome, autosomal recessive congenital 5 |
| AGL | Glycogen storage disease, Glycogen storage disease IIIa, Glycogen storage disease |
| | IIIb, Glycogen storage disease type III |
| AGPAT2 | Congenital generalized lipodystrophy type 1 |
| AGRN | Congenital myasthenic syndrome |
| AGT | Renal dysplasia |
| AGTR1 | Renal dysplasia |
| AGXT | Primary hyperoxaluria, type I |
| AHDC1 | Delayed speech and language development, Global developmental delay, |
| | Intellectual disability, Muscular hypotonia, Neonatal hypotonia, Sleep apnea, Xia- |
| | Gibbs syndrome |
| AHI1 | Joubert syndrome, Joubert syndrome 3, Retinal dystrophy, Retinitis pigmentosa |
| AHR | Retinitis pigmentosa 85 |
| AIRE | Autoimmune polyglandular syndrome type 1, Polyglandular autoimmune |
| | syndrome, type 1, with reversible metaphyseal dysplasia |
| ALB | Analbuminemia |
| ALDH18A1 | Cutis laxa-corneal clouding-oligophrenia syndrome |
| ALDH3A2 | SjÃ¶gren-Larsson syndrome |
| ALDH5A1 | Succinate-semialdehyde dehydrogenase deficiency |
| ALDH7A1 | Pyridoxine-dependent epilepsy, Seizures |
| ALDOB | Hereditary fructosuria |
| ALG1 | ALG1-CDG, Congenital disorder of glycosylation |
| ALG3 | ALG3-CDG |
| ALMS1 | Alstrom syndrome |
| ALOX12B | Autosomal recessive congenital ichthyosis 2 |
| ALPK3 | CARDIOMYOPATHY, FAMILIAL HYPERTROPHIC 27, Hypertrophic |
| | cardiomyopathy |
| ALPL | Hypophosphatasia, Infantile hypophosphatasia |
| ALS2 | Amyotrophic lateral sclerosis type 2, Infantile-onset ascending hereditary spastic |
| | paralysis, Juvenile primary lateral sclerosis |
| ALX4 | Parietal foramina 2 |
| AMPD2 | Pontocerebellar hypoplasia, type 9 |
| AMT | Non-ketotic hyperglycinemia |
| ANAPC1 | Rothmund-Thomson syndrome type 1 |
| ANGPTL3, | 2, Hypobetalipoproteinemia, familial |
| DOCK7 | |
| ANKRD1 | ANKRD1-related dilated cardiomyopathy, Cardiovascular phenotype, Primary |
| | dilated cardiomyopathy |
| ANKRD11 | Abnormal facial shape, Clinodactyly of the 5th finger, Conductive hearing |
| | impairment, Delayed speech and language development, Global developmental |
| | delay, Inborn genetic diseases, Intellectual disability, KBG syndrome, Ptosis, |
| | Seizures, Short foot, Short palm, Unilateral cryptorchidism |
| ANO10 | Autosomal recessive cerebellar ataxia, Spinocerebellar ataxia, autosomal recessive |
| | 10 |
| ANO5 | ANO5-Related Disorders, Achilles tendon contracture, Elevated serum creatine |
| | phosphokinase, Gnathodiaphyseal dysplasia, Limb-girdle muscular dystrophy, |
| | Lower limb amyotrophy, Lower limb muscle weakness, Miyoshi muscular |
| | dystrophy 3, Muscular Diseases, Polycystic kidney dysplasia, type 2L |
| ANTXR1 | Odontotrichomelic syndrome |
| AP1B1 | Autosomal recessive keratitis-ichthyosis-deafhess syndrome |
| AP3B1 | Hermansky-Pudlak syndrome 2 |
| AP4B1, AP4B1- | Inborn genetic diseases, Spastic paraplegia 47, autosomal recessive |
| AS1 | |
| AP4M1 | Spastic paraplegia 50, autosomal recessive |
| AP5Z1 | Spastic paraplegia 48, autosomal recessive |
| APC | Adenomatous colonic polyposis, Adenomatous polyposis coli with congenital |
| | cholesteatoma, Brain tumor-polyposis syndrome 2, Carcinoma of colon, Colon |
| | adenocarcinoma, Colorectal cancer, Craniopharyngioma, Desmoid disease, |
| | Desmoid tumors, Duodenal polyposis, Familial adenomatous polyposis, Familial |
| | adenomatous polyposis 1, Familial multiple polyposis syndrome, Gardner |
| | syndrome, Gastric polyposis, Hepatocellular carcinoma, Hereditary cancer- |
| | predisposing syndrome, Hyperplastic colonic polyposis, Intestinal polyp, |
| | Malignant Colorectal Neoplasm, Neoplasm of stomach, Neoplasm of the large |
| | intestine, Periampullary adenoma, hereditary, susceptibility to |
| APOA1, APOA1- | Familial hypoalphalipoproteinemia |
| AS | |
| APOB | 1, Familial hypobetalipoproteinemia, Hypobetalipoproteinemia, familial, |
| | normotriglyceridemic |
| APOC2 | APOLIPOPROTEIN C-II (NIJMEGEN), Apolipoprotein C2 deficiency |
| APOC2, APOC4- | APOLIPOPROTEIN C-II (PADOVA), Apolipoprotein C2 deficiency |
| APOC2 | |
| APTX | Ataxia-oculomotor apraxia type 1 |
| AR | Androgen resistance syndrome, Bulbo-spinal atrophy X-linked, Partial androgen |
| | insensitivity syndrome |
| ARCN1 | Short stature, and developmental delay, micrognathia, rhizomelic, with |
| | microcephaly |
| ARG1, MED23 | Arginase deficiency |
| ARHGEF18 | Retinitis pigmentosa 78 |
| ARID1A | Mental retardation, autosomal dominant 14 |
| ARID1B | Absent speech, Blepharophimosis, Coffin-Siris syndrome 1, Constipation, |
| | Decreased body weight, Failure to thrive, Inborn genetic diseases, Intellectual |
| | disability, Long eyelashes, Microcephaly, Recurrent respiratory infections, |
| | Seizures, Short stature, Thick lower lip vermilion, Thin upper lip vermilion, |
| | moderate |
| ARID2 | COFFIN-SIRIS SYNDROME 6 |
| ARL2BP | Retinitis pigmentosa 82 with or without situs inversus |
| ARMC2 | Male infertility with teratozoospermia due to single gene mutation, |
| | SPERMATOGENIC FAILURE 38, Sperm tail anomaly |
| ARMC2, | Male infertility with teratozoospermia due to single gene mutation, |
| ARMC2-AS1 | SPERMATOGENIC FAILURE 38 |
| ARMC5 | Acth-independent macronodular adrenal hyperplasia 2 |
| ARSA | Metachromatic leukodystrophy, Pseudoarylsulfatase A deficiency, late infantile |
| ARSB | Metachromatic leukodystrophy, Mucopolysaccharidosis type 6 |
| ART4 | Blood group, Dombrock system |
| ASAH1 | Farber disease, Spinal muscular atrophy-progressive myoclonic epilepsy syndrome |
| ASL | Argininosuccinate lyase deficiency |
| ASPA, SPATA22 | Canavan Disease, Familial Form, Spongy degeneration of central nervous system |
| ASPM | Microcephaly, Primary autosomal recessive microcephaly, Primary autosomal |
| | recessive microcephaly 1, Primary autosomal recessive microcephaly 5 |
| ASS1 | Citrullinemia type I |
| ASXL1 | Bohring-Opitz syndrome, Inborn genetic diseases |
| ASXL3 | Bainbridge-Ropers syndrome |
| ATF6 | Achromatopsia, Achromatopsia 7 |
| ATL1 | Hereditary spastic paraplegia 3A |
| ATM | Ataxia-telangiectasia syndrome, Familial cancer of breast, Hereditary breast and |
| | ovarian cancer syndrome, Hereditary cancer-predisposing syndrome, Ovarian |
| | Neoplasms |
| ATM, C11orf65, | Ataxia-telangiectasia syndrome, Ataxia-telangiectasia without immunodeficiency, |
| ATP13A2 | Breast cancer, Familial cancer of breast, Hereditary breast and ovarian cancer |
| | syndrome, Hereditary cancer-predisposing syndrome, Neoplasm of the breast, |
| | susceptibility to Kufor-Rakeb syndrome |
| ATP1A2 | Abnormality of neuronal migration, Arthrogryposis multiplex congenita, Epilepsy, |
| | Hydrops fetalis |
| ATP2A1 | Brody myopathy |
| ATP2C1 | Familial benign pemphigus |
| ATP6V0A2 | ALG9 congenital disorder of glycosylation, Cutis laxa with osteodystrophy |
| ATP6V0A4 | Renal tubular acidosis, autosomal recessive, distal |
| ATP7A | Cutis laxa, Menkes kinky-hair syndrome, X-linked |
| ATP7B | Inborn genetic diseases, Wilson disease |
| ATRX | 1, Alpha thalassemia-X-linked intellectual disability syndrome, Intellectual |
| | disability, Mental retardation-hypotonic facies syndrome, Mental retardation- |
| | hypotonic facies syndrome X-linked, X-linked |
| AXIN2 | Oligodontia-colorectal cancer syndrome |
| B3GALNT1 | p phenotype |
| B3GALNT2 | 11, Muscular dystrophy-dystroglycanopathy (congenital with brain and eye |
| | anomalies), type a |
| B3GALT6 | Spondylo-epi-(meta)-physeal dysplasia |
| B4GALNT1 | Hereditary spastic paraplegia 26, Inborn genetic diseases |
| B4GALT7 | Ehlers-Danlos syndrome progeroid type |
| B9D1 | Joubert syndrome, Meckel syndrome, Meckel-Gruber syndrome, type 9 |
| B9D2 | Joubert syndrome |
| BAG3 | BAG3-related, Cardiovascular phenotype, Dilated cardiomyopathy 1HH, Inborn |
| | genetic diseases, Myofibrillar myopathy, Primary dilated cardiomyopathy |
| BAP1 | Hereditary cancer-predisposing syndrome, Tumor susceptibility linked to germline |
| | BAP1 mutations |
| BARD1 | Breast cancer, Familial cancer of breast, Hereditary breast and ovarian cancer |
| | syndrome, Hereditary cancer-predisposing syndrome, Triple-Negative Breast |
| | Cancer Finding, susceptibility to |
| BBS1 | Bardet-Biedl syndrome |
| BBS1, ZDHHC24 | Bardet-Biedl syndrome, Bardet-Biedl syndrome 1 |
| BBS10 | Bardet-Biedl syndrome, Bardet-Biedl syndrome 1, Bardet-Biedl syndrome 10, |
| | Bardet-biedl syndrome 6/10, Inborn genetic diseases, Retinal dystrophy, Retinitis |
| | pigmentosa, digenic |
| BBS2 | Bardet-Biedl syndrome, Bardet-Biedl syndrome 2, Bardet-biedl syndrome 1/2, |
| | Bardet-biedl syndrome 2/6, Retinal dystrophy, Retinitis pigmentosa, Retinitis |
| | pigmentosa 74, digenic |
| BBS5 | Bardet-Biedl syndrome 5 |
| BBS9 | Bardet-Biedl syndrome |
| BCKDHA | Maple syrup urine disease, Maple syrup urine disease type 1A |
| BCKDHB | CLASSIC, MAPLE SYRUP URINE DISEASE, Maple syrup urine disease, Maple |
| | syrup urine disease type 1B, TYPE IB |
| BCOR | Oculofaciocardiodental syndrome |
| BCS1L | BCS1L-Related Disorders, GRACILE syndrome, Leigh syndrome, Mitochondrial |
| | complex III deficiency, Pili torti-deafness syndrome, nuclear type 1 |
| BEST1 | Bestrophinopathy, Retinal dystrophy, Vitelliform macular dystrophy type 2, |
| | autosomal recessive |
| BET1 | Progressive muscle weakness, Seizures |
| BFSP1 | Cataract 33, multiple types |
| BLM | Bloom syndrome, Hereditary breast and ovarian cancer syndrome, Hereditary |
| | cancer-predisposing syndrome |
| BMP1 | Osteogenesis imperfecta, type xiii |
| BMP2 | AND SKELETAL ANOMALIES WITH OR WITHOUT CARDIAC |
| | ANOMALIES, FACIAL DYSMORPHISM, SHORT STATURE |
| BMPR1A | Hereditary cancer-predisposing syndrome, Juvenile polyposis syndrome |
| BMPR2 | Primary pulmonary hypertension |
| BNC1 | PREMATURE OVARIAN FAILURE 16 |
| BOLA3 | Multiple mitochondrial dysfunctions syndrome 2 |
| BPNT2 | Chondrodysplasia with joint dislocations, GPAPP type |
| BPTF | NEURODEVELOPMENTAL DISORDER WITH DYSMORPHIC FACIES AND |
| | DISTAL LIMB ANOMALIES |
| BRAT1 | Inborn genetic diseases, NEURODEVELOPMENTAL DISORDER WITH |
| | CEREBELLAR ATROPHY AND WITH OR WITHOUT SEIZURES, Rigidity |
| | and multifocal seizure syndrome, lethal neonatal |
| BRCA1 | Breast and/or ovarian cancer, Breast carcinoma, Breast-ovarian cancer, |
| | COMPLEMENTATION GROUP S, Dysgerminoma, FANCONI ANEMIA, |
| | Familial cancer of breast, Hereditary breast and ovarian cancer syndrome, |
| | Hereditary cancer-predisposing syndrome, Infiltrating duct carcinoma of breast, |
| | Neoplasm of ovary, Neoplasm of the breast, Ovarian Neoplasms, Ovarian Serous |
| | Surface Papillary Adenocarcinoma, Ovarian cancer, Pancreatic cancer, Pancreatic |
| | cancer 4, Porokeratosis punctata palmaris et plantaris, Rhabdomyosarcoma |
| | (disease), bilateral breast cancer, breast cancer, familial 1, susceptibility to |
| BRCA2 | Asthma, BRCA2-Related Disorders, Breast and/or ovarian cancer, Breast |
| | carcinoma, Breast-ovarian cancer, Cancer of the pancreas, Colorectal cancer, |
| | Diffuse intrinsic pontine glioma, Ectopic ossification, Familial cancer of breast, |
| | Fanconi anemia, Focal seizures, Genetic non-acquired premature ovarian failure, |
| | Glioma susceptibility 3, Headache, Hereditary Cancer Syndrome, Hereditary |
| | breast and ovarian cancer syndrome, Hereditary cancer-predisposing syndrome, |
| | Inborn genetic diseases, Malignant tumor of prostate, Medulloblastoma, Migraine, |
| | Muscle weakness, Neoplasm of the breast, Nephrolithiasis, Obesity, Ovarian |
| | Neoplasms, Ovarian cancer, Pancreatic cancer 2, Polydactyly, Short attention span, |
| | Striae distensae, Tracheoesophageal fistula, Tumor susceptibility linked to |
| | germline BAP1 mutations, Wilms tumor 1, complementation group D1, familial 1, |
| | familial 2 |
| BRIP1 | BRIP1-Related Disorders, Breast cancer, Carcinoma of colon, Familial cancer of |
| | breast, Fanconi anemia, Hereditary breast and ovarian cancer syndrome, |
| | Hereditary cancer-predisposing syndrome, Neoplasm of ovary, Neoplasm of the |
| | breast, Ovarian Cancers, Ovarian Neoplasms, Tracheoesophageal fistula, |
| | complementation group J, early-onset |
| BRWD3 | Mental retardation, X-linked 93 |
| BSND | Bartter disease type 4a |
| BTD | Biotinidase deficiency |
| BTK | Agammaglobulinemia, X-linked agammaglobulinemia, X-linked |
| | agammaglobulinemia with growth hormone deficiency, non-Bruton type |
| C11orf65, ATM | Ataxia-telangiectasia syndrome, Hereditary breast and ovarian cancer syndrome, |
| | Hereditary cancer-predisposing syndrome |
| C12orf4 | AUTOSOMAL RECESSIVE 66, Attention deficit hyperactivity disorder, |
| | Intellectual disability, MENTAL RETARDATION, Muscular hypotonia |
| C12orf65 | Combined oxidative phosphorylation deficiency 7, Spastic paraplegia |
| C19orf12 | Neurodegeneration with brain iron accumulation 4, Spastic paraplegia 43, |
| | autosomal recessive |
| C1QB | C1q deficiency |
| C1S | Complement component c1s deficiency |
| C2 | Complement component 2 deficiency |
| C2CD3 | Orofaciodigital syndrome xiv |
| C5 | Leiner disease |
| C6 | Complement component 6 deficiency, Immunodeficiency due to a late component |
| | of complement deficiency |
| C7 | Complement component 7 deficiency |
| C8B | Complement component 6 deficiency, Type II complement component 8 |
| | deficiency |
| C8orf37 | Cone-rod dystrophy 16 |
| C8orf37 | Retinitis pigmentosa 64 |
| CA2 | Osteopetrosis with renal tubular acidosis |
| CABP4 | Congenital stationary night blindness, type 2B |
| CACNA1A | 42, Bulbar palsy, Epileptic encephalopathy, Episodic ataxia, Episodic ataxia type |
| | 2, Recurrent respiratory infections, and epilepsy, early infantile, type 2 |
| CACNA1C | Long QT syndrome |
| CACNA2D4 | Abnormality of the eye, Retinal cone dystrophy 4 |
| CAPN1 | Spastic paraplegia 76, autosomal recessive |
| CAPN3 | Absent Achilles reflex, Absent muscle fiber calpain-3, Arrhythmia, Calf muscle |
| | hypertrophy, Congenital muscular dystrophy, Contractures of the joints of the |
| | lower limbs, Difficulty walking, EMG: myopathic abnormalities, EMG: |
| | neuropathic changes, Elbow flexion contracture, Elevated serum creatine |
| | phosphokinase, Limb-Girdle Muscular Dystrophy, Limb-girdle muscle weakness, |
| | Limb-girdle muscular dystrophy, Migraine, Muscle weakness, Muscular Diseases, |
| | Muscular dystrophy, Myositis, Paresthesia, Positive Romberg sign, Progressive |
| | spinal muscular atrophy, Recessive, Shoulder girdle muscle weakness, |
| | eosinophilic, type 2A |
| CASK | Mental retardation and microcephaly with pontine and cerebellar hypoplasia |
| CASP14 | Ichthyosis, autosomal recessive 12, congenital |
| CASQ2 | 2, Ventricular tachycardia, catecholaminergic polymorphic |
| CASR | Hypocalciuric hypercalcemia, Inborn genetic diseases, familial, type 1 |
| CAST | Peeling skin with leukonychia, acral punctate keratoses, and knuckle pads, cheilitis |
| CAST, ERAP1 | Peeling skin with leukonychia, acral punctate keratoses, and knuckle pads, cheilitis |
| CAT | Acatalasemia, Acatalasia, Japanese type |
| CATSPER1 | Spermatogenic failure 7 |
| CAV1 | Lipoqdystrophy, congenital generalized, type 3 |
| CAV3, SSUH2 | Long QT syndrome |
| CBL | Noonan syndrome-like disorder with or without juvenile myelomonocytic |
| | leukemia |
| CBS | CYSTATHIONINE BETA-SYNTHETASE POLYMORPHISM, Classic |
| | homocystinuria, Homocystinuria |
| CC2D1A | Mental Retardation, Mental retardation, Psychosocial, autosomal recessive 3 |
| CC2D2A | Joubert syndrome, Joubert syndrome 9, Meckel syndrome type 6, Meckel-Gruber |
| | syndrome |
| CCBE1 | Hennekam lymphangiectasia-lymphedema syndrome 1 |
| CCDC103 | Primary ciliary dyskinesia |
| CCDC28B | Bardet-Biedl syndrome, Bardet-Biedl syndrome 1, modifier of |
| CCDC39 | 14, Ciliary dyskinesia, Primary ciliary dyskinesia, primary |
| CCDC40 | 15, Ciliary dyskinesia, Primary ciliary dyskinesia, primary |
| CCDC47 | Global developmental delay with dysmorphic features, |
| | Trichohepatoneurodevelopmental syndrome, and woolly hair, liver dysfunction, |
| | pruritus |
| CCDC65 | 27, Ciliary dyskinesia, Kartagener syndrome, Primary ciliary dyskinesia, primary |
| CCDC78 | 4, Myopathy, centronuclear |
| CCDC88C | Congenital hydrocephalus 1 |
| CCN6 | Progressive pseudorheumatoid dysplasia |
| CCNH, RASA1 | Capillary malformation-arteriovenous malformation |
| CCNO | 29, Ciliary dyskinesia, Kartagener syndrome, Primary ciliary dyskinesia, primary |
| CCNQ | Syndactyly-telecanthus-anogenital and renal malformations syndrome |
| CD19 | Common variable immunodeficiency 3 |
| CD247 | Immunodeficiency due to defect in cd3-zeta |
| CD36 | Malaria, Platelet glycoprotein IV deficiency, cerebral, susceptibility to |
| CD46 | Atypical hemolytic-uremic syndrome 2 |
| CD55 | CROMER BLOOD GROUP SYSTEM, Dr(a-) PHENOTYPE, Protein-losing |
| | enteropathy (disease) |
| CDC14A | Deafness, Rare genetic deafness, autosomal recessive 32 |
| CDC73 | Parathyroid adenoma, Parathyroid carcinoma |
| CDH1 | Blepharocheilodontic syndrome 1, Breast cancer, Endometrial carcinoma, Familial |
| | cancer of breast, Hereditary cancer-predisposing syndrome, Hereditary diffuse |
| | gastric cancer, Malignant tumor of prostate, Neoplasm of ovary, lobular |
| CDH11 | Brachioskeletogenital syndrome |
| CDH23 | Deafness, Inborn genetic diseases, MULTIPLE TYPES, PITUITARY |
| | ADENOMA 5, Rare genetic deafness, Usher syndrome type 1D, autosomal |
| | recessive 12 |
| CDH23, | Rare genetic deafness |
| C10orf105 | |
| CDH23, CDH23- | DIGENIC, TYPE ID/F, USHER SYNDROME, Usher syndrome type 1, Usher |
| AS1 | syndrome type 1D |
| CDH3 | Congenital hypotrichosis with juvenile macular dystrophy, EEM syndrome, |
| | Hypotrichosis with juvenile macular dystrophy, Macular dystrophy |
| CDHR1 | Cone-rod dystrophy 15, Leber congenital amaurosis, Retinal dystrophy, Retinitis |
| | pigmentosa 65 |
| CDK10 | AL KAISSI SYNDROME |
| CDK13 | Congenital heart defects, and intellectual developmental disorder, dysmorphic |
| | facial features |
| CDK5RAP2 | Primary autosomal recessive microcephaly 3 |
| CDKL5 | Angelman syndrome-like, Atypical Rett syndrome, Early infantile epileptic |
| | encephalopathy 2, Epileptic encephalopathy, Inborn genetic diseases |
| CDKN2A | Hereditary cancer-predisposing syndrome, Hereditary cutaneous melanoma, |
| | Melanoma-pancreatic cancer syndrome, Neoplasm |
| CDSN, | Peeling skin syndrome 1 |
| PSORS1C1 | |
| CEL | Maturity-onset diabetes of the young type 8 |
| CELA2A | Coronary artery disease, Diabetes, Familial partial lipodystrophy 6, Hypertensive |
| | disorder, Hypertriglyceridemia |
| CENPF | Stromme syndrome |
| CENPJ | Congenital microcephaly, Intellectual disability, Perisylvian polymicrogyria, |
| | Primary autosomal recessive microcephaly, Primary autosomal recessive |
| | microcephaly 1, Primary autosomal recessive microcephaly 6, Seckel syndrome 4, |
| | Type III lissencephaly, moderate |
| CEP120 | JOUBERT SYNDROME 31 |
| CEP152 | Seckel syndrome |
| CEP290 | Abnormality of the kidney, Bardet-Biedl syndrome 14, Blindness, CEP290- |
| | Related Disorders, Cerebellar cyst, Cerebellar vermis hypoplasia, Global |
| | developmental delay, Hyperechogenic kidneys, Joubert syndrome, Joubert |
| | syndrome 5, Leber congenital amaurosis 10, Meckel syndrome, Meckel-Gruber |
| | syndrome, Nephronophthisis, Polycystic kidney dysplasia, Retinal dystrophy, |
| | Senior-Loken syndrome 6, type 4 |
| CEP290, | Bardet-Biedl syndrome 14, Joubert syndrome, Joubert syndrome 5, Meckel-Gruber |
| C12orf29 | syndrome, Nephronophthisis |
| CEP41 | Joubert syndrome 15 |
| CEP78 | Cone-rod degeneration, Cone-rod dystrophy and hearing loss 1, Sensorineural |
| | hearing loss |
| CFAP251 | Male infertility with teratozoospermia due to single gene mutation, Non-syndromic |
| | male infertility due to sperm motility disorder, SPERMATOGENIC FAILURE 18, |
| | SPERMATOGENIC FAILURE 33, asthenozoospermia, dysplasia of the |
| | mitochondrial sheath, multiple morphologic abnormalities of the sperm flagellum |
| CFAP410 | Axial spondylometaphyseal dysplasia, RETINAL DYSTROPHY WITH OR |
| | WITHOUT MACULAR STAPHYLOMA |
| CFAP43 | SPERMATOGENIC FAILURE 19 |
| CFAP44 | SPERMATOGENIC FAILURE 20 |
| CFHR5 | CFHR5 deficiency |
| CFTR | Bronchiectasis with or without elevated sweat chloride 1, CFTR-related disorders, |
| | Congenital bilateral aplasia of vas deferens from CFTR mutation, Cystic fibrosis, |
| | Hereditary pancreatitis, Inborn genetic diseases, ataluren response - Efficacy |
| CFTR, CFTR- | CFTR-related disorders, Congenital bilateral aplasia of vas deferens from CFTR |
| AS1 | mutation, Cystic fibrosis |
| CFTR, | Bronchiectasis with or without elevated sweat chloride 1, CFTR-related disorders, |
| LOC111674472 | Congenital bilateral aplasia of vas deferens from CFTR mutation, Cystic fibrosis, |
| | Hereditary pancreatitis |
| CFTR, | Bronchiectasis with or without elevated sweat chloride 1, CFTR-related disorders, |
| LOC111674475 | Congenital bilateral aplasia of vas deferens from CFTR mutation, Cystic fibrosis, |
| | Hereditary pancreatitis, Inborn genetic diseases, ataluren response - Efficacy |
| CFTR, | Cystic fibrosis |
| LOC111674477 | |
| CFTR, | Bronchiectasis with or without elevated sweat chloride 1, CFTR-related disorders, |
| LOC113633877 | Congenital bilateral aplasia of vas deferens from CFTR mutation, Cystic fibrosis, |
| | Hereditary pancreatitis |
| CFTR, | Bronchiectasis with or without elevated sweat chloride 1, Congenital bilateral |
| LOC113664106 | aplasia of vas deferens from CFTR mutation, Cystic fibrosis, Hereditary |
| | pancreatitis |
| CHD2 | CHD2-Related Disorder, Epileptic encephalopathy, History of neurodevelopmental |
| | disorder, childhood-onset |
| CHD7 | CHARGE association, Hypogonadism with anosmia, Hypogonadotropic |
| | hypogonadism 5 with or without anosmia |
| CHEK2 | 3, Astrocytoma, B Lymphoblastic Leukemia/Lymphoma, Breast and colorectal |
| | cancer, Breast cancer, CHEK2-Related Cancer Susceptibility, Colitis, Congenital |
| | heart defects, Diffuse intrinsic pontine glioma, Familial cancer of breast, |
| | Hematochezia, Hereditary breast and ovarian cancer syndrome, Hereditary cancer, |
| | Hereditary cancer-predisposing syndrome, Inflammation of the large intestine, |
| | Leiomyosarcoma, Li-Fraumeni syndrome, Li-Fraumeni syndrome 2, Malignant |
| | tumor of prostate, Neoplasm of the breast, Not Otherwise Specified, |
| | Osteosarcoma, Ovarian Neoplasms, Prostate cancer, Thrombocytopenia, multiple |
| | types, somatic, susceptibility to |
| CHM | Retinal dystrophy |
| CHRDL1 | Megalocornea |
| CHRNA1 | Congenital myasthenic syndrome |
| CHRNA2 | Autosomal dominant nocturnal frontal lobe epilepsy |
| CHRNA3 | CHRNA3-related condition |
| CHRND | Lethal multiple pterygium syndrome |
| CHRNE | 4a, Congenital myasthenic syndrome, Congenital myasthenic syndrome 4C, |
| | Myasthenic syndrome, congenital, slow-channel |
| CHRNE, | 4a, 4b, Congenital myasthenic syndrome, Congenital myasthenic syndrome 4C, |
| C17orf107 | Myasthenic syndrome, congenital, fast-channel, slow-channel |
| CHRNG | Autosomal recessive multiple pterygium syndrome, CHRNG-Related Disorders, |
| | Inborn genetic diseases, Lethal multiple pterygium syndrome |
| CHST14 | Ehlers-Danlos syndrome, musculocontractural type |
| CHST3 | Spondyloepiphyseal dysplasia with congenital joint dislocations |
| CHSY1 | Temtamy preaxial brachydactyly syndrome |
| CIB1 | 3, EPIDERMODYSPLASIA VERRUCIFORMIS, SUSCEPTIBILITY TO |
| CIITA | Bare lymphocyte syndrome 2 |
| CKAP2L | Filippi syndrome |
| CLCN1 | Autosomal dominant intermediate Charcot-Marie-Tooth disease, Congenital |
| | myotonia, EMG: myopathic abnormalities, Muscular Diseases, Myotonia |
| | congenita, autosomal dominant form, autosomal recessive form |
| CLCN2 | Epilepsy, Leukoencephalopathy with ataxia, juvenile myoclonic 8 |
| CLCN5 | Nephrolithiasis, X-linked recessive, X-linked recessive nephrolithiasis with renal |
| | failure |
| CLDN1, CLDN16 | Neonatal ichthyosis-sclerosing cholangitis syndrome |
| CLIC5 | Deafness, autosomal recessive |
| CLN3 | Juvenile neuronal ceroid lipofuscinosis, Neuronal ceroid lipofuscinosis |
| CLN5, FBXL3 | Neuronal ceroid lipofuscinosis, Neuronal ceroid lipofuscinosis 5 |
| CLRN1 | Rare genetic deafness, Retinal dystrophy, Retinitis pigmentosa, Usher syndrome, |
| | type 3A |
| CNGA1, | Retinal dystrophy, Retinitis pigmentosa 49 |
| LOC101927157 | |
| CNGB1 | Retinal dystrophy, Retinitis pigmentosa, Retinitis pigmentosa 45 |
| CNGB3 | Abnormality of the eye, Achromatopsia, Achromatopsia 3, CNGB3-Related |
| | Disorders, Cone-rod dystrophy, Leber congenital amaurosis, Recessive, Retinal |
| | dystrophy, Retinitis pigmentosa, Stargardt Disease |
| CNNM2 | Hypomagnesemia 6, renal |
| CNNM4 | Jalili syndrome |
| CNTNAP1 | Lethal congenital contracture syndrome 7 |
| CNTNAP2 | Pitt-Hopkins-like syndrome 1 |
| COASY | Neurodegeneration with brain iron accumulation 6 |
| COG4 | Congenital disorder of glycosylation type 2J |
| COG5 | Congenital disorder of glycosylation type 2i |
| COG5, DUS4L, | Congenital disorder of glycosylation type 2i |
| DUS4L-BCAP29 | |
| COL10A1 | Metaphyseal chondrodysplasia, Schmid type |
| COL11A1 | Fibrochondrogenesis 1 |
| COL12A1 | Ullrich congenital muscular dystrophy 2 |
| COL17A1 | Epidermolysis bullosa, Junctional epidermolysis bullosa, junctional, localisata |
| | variant, non-Herlitz type |
| COL18A1 | GLAUCOMA, Knobloch syndrome 1, PRIMARY CLOSED-ANGLE |
| COL18A1, | Knobloch syndrome 1, Macular dystrophy, Retinal dystrophy, Retinitis pigmentosa |
| SLC19A1 | |
| COL1A1 | Ehlers-Danlos syndrome, Infantile cortical hyperostosis, Osteogenesis imperfecta, |
| | Osteogenesis imperfecta type I, Osteogenesis imperfecta type III, Osteogenesis |
| | imperfecta with normal sclerae, Postmenopausal osteoporosis, dominant form, |
| | procollagen proteinase deficient, recessive perinatal lethal |
| COL1A2 | COL1A2-Related Disorder, Ehlers-Danlos syndrome, Inborn genetic diseases, |
| | Osteogenesis imperfecta type I, autosomal recessive, cardiac valvular form, classic |
| | type |
| COL2A1 | Spondyloperipheral dysplasia-short ulna syndrome, Stickler syndrome type 1 |
| COL3A1 | Ehlers-Danlos syndrome, type 4 |
| COL4A3, MFF- | Alport syndrome, autosomal recessive |
| DT | |
| COL4A5 | Alport syndrome 1, X-linked recessive |
| COL5A1 | Ehlers-Danlos syndrome, classic type |
| COL5A2 | Ehlers-Danlos syndrome, Ehlers-Danlos syndrome classic type 2, classic type |
| COL6A1 | Bethlem myopathy 1 |
| COL6A2 | Bethlem myopathy 1, Ullrich congenital muscular dystrophy 1 |
| COL6A3 | Bethlem myopathy 1 |
| COL7A1 | Dystrophic epidermolysis bullosa, Epidermolysis bullosa pruriginosa, Recessive |
| | dystrophic epidermolysis bullosa, Transient bullous dermolysis of the newborn, |
| | autosomal dominant |
| COL9A2 | Stickler syndrome, type 5 |
| COLEC10 | 3MC syndrome 3 |
| COLEC10, | 3MC syndrome 3 |
| LOC101927513 | |
| COLQ | Congenital myasthenic syndrome, Endplate acetylcholinesterase deficiency |
| COQ2 | Coenzyme Q10 deficiency, primary, primary 1 |
| COQ8A | 4, ADCK3-Related Disorders, Coenzyme Q10 deficiency, primary |
| COQ9 | 5, Coenzyme Q10 deficiency, primary |
| COX15 | Cardioencephalomyopathy, Leigh syndrome, Leigh syndrome due to |
| | mitochondrial complex IV deficiency, due to cytochrome c oxidase deficiency 2, |
| | fatal infantile |
| CP | Ceruloplasmin belfast, Deficiency of ferroxidase, Hemosiderosis, due to |
| | aceruloplasminemia, systemic |
| CPAMD8 | Anterior segment dysgenesis 8 |
| CPLANE1 | Global developmental delay, Jaundice, Joubert syndrome, Joubert syndrome 1, |
| | Joubert syndrome 17, Orofaciodigital syndrome type 6, Typical Joubert syndrome |
| | MRI findings |
| CPOX | Coproporphyria |
| CPS1 | Congenital hyperammonemia, type I |
| CPSF1 | MYOPIA 27 |
| CPT2 | Carnitine palmitoyltransferase II deficiency, infantile, lethal neonatal, myopathic, |
| | stress-induced |
| CRB1 | Leber congenital amaurosis 8 |
| CRB2 | Focal segmental glomerulosclerosis 9, Steroid-resistant nephrotic syndrome |
| CRIPT | Ateleiotic dwarfism, Short stature with microcephaly and distinctive facies |
| CRPPA | 7, Congenital muscular dystrophy-dystroglycanopathy with brain and eye |
| | anomalies, Muscular dystrophy-dystroglycanopathy (limb-girdle), type A7, type c |
| CRTAP | Osteogenesis imperfecta type 7 |
| CRX | Leber congenital amaurosis 7 |
| CRYAB | Alpha-B crystallinopathy, Dilated cardiomyopathy 1II |
| CRYBA4, | Cataract, autosomal recessive 3, congenital nuclear |
| CRYBB1 | |
| CRYBB2 | Cataract 3, Congenital cataract, multiple types |
| CSGALNACT1 | MILD, SKELETAL DYSPLASIA, WITH JOINT LAXITY AND ADVANCED |
| | BONE AGE |
| CSPP1 | Joubert syndrome 21, Meckel-Gruber syndrome |
| CSRP3 | Cardiovascular phenotype |
| CSTB | Inborn genetic diseases, Progressive myoclonic epilepsy, Unverricht-Lundborg |
| | syndrome |
| CTC1 | Cerebroretinal microangiopathy with calcifications and cysts, Cerebroretinal |
| | microangiopathy with calcifications and cysts 1, Dyskeratosis congenita |
| CTCF | Mental retardation, autosomal dominant 21 |
| CTNNB1 | EXUDATIVE VITREORETINOPATHY 7, Exudative vitreoretinopathy 1, |
| | Hepatocellular carcinoma, Inborn genetic diseases, Mental retardation, autosomal |
| | dominant 19 |
| CTNND1, TMX2- | Blepharocheilodontic syndrome 2 |
| CTNND1 | |
| CTNS | Cystinosis, Juvenile nephropathic cystinosis, Nephropathic cystinosis, Ocular |
| | cystinosis |
| CTSD | Neuronal ceroid lipofuscinosis 10 |
| CTSH | Variant of unknown significance |
| CTU2 | AND AMBIGUOUS GENITALIA SYNDROME, FACIAL DYSMORPHISM, |
| | MICROCEPHALY, RENAL AGENESIS |
| CUBN | Megaloblastic anemia due to inborn errors of metabolism |
| CUL4B | Cabezas type, Syndromic X-linked mental retardation |
| CUL7 | Three M syndrome 1 |
| CWC27 | Retinitis pigmentosa with or without skeletal anomalies |
| CWF19L1 | Spinocerebellar ataxia, autosomal recessive 17 |
| CYB5R3 | Methemoglobinemia type 2 |
| CYBB | Chronic granulomatous disease, X-linked |
| CYP11B1, | Deficiency of steroid 11-beta-monooxygenase |
| LOC106799833 | |
| CYP17A1 | 20-lyase deficiency, Combined partial 17-alpha-hydroxylase/17, Complete |
| | combined 17-alpha-hydroxylase/17, Deficiency of steroid 17-alpha- |
| | monooxygenase |
| CYP1B1 | A, Anterior segment dysgenesis 6, CYP1B1-Related Disorders, Congenital |
| | glaucoma, Congenital ocular coloboma, Glaucoma, Glaucoma 3, Irido-corneo- |
| | trabecular dysgenesis, b, congenital, primary congenital, primary infantile |
| CYP21A2, | Classic congenital adrenal hyperplasia due to 21-hydroxylase deficiency |
| LOC106780800 | |
| CYP21A2, | Classic congenital adrenal hyperplasia due to 21-hydroxylase deficiency |
| TNXB, | |
| LOC106780800 | |
| CYP24A1 | 1, Hypercalcemia, infantile |
| CYP26C1 | Optic nerve hypoplasia |
| CYP27A1 | Cholestanol storage disease |
| CYP27B1 | Vitamin D-dependent rickets, type 1 |
| CYP2C19 | CYP2C19: no function, Clopidogrel response, Mephenytoin, Proguanil, |
| | Toxicity/ADR, amitriptyline response - Efficacy, citalopram response - Efficacy, |
| | clomipramine response - Efficacy, clopidogrel response - Efficacy, poor |
| | metabolism of |
| CYP2D6 | Debrisoquine, Deutetrabenazine response, Tamoxifen response, Toxicity/ADR, |
| | Tramadol response, amitriptyline response - Dosage, antidepressants response - |
| | Dosage, clomipramine response - Dosage, desipramine response - Dosage, doxepin |
| | response - Dosage, imipramine response - Dosage, nortriptyline response - Dosage, |
| | poor metabolism of, tamoxifen response - Efficacy, trimipramine response - |
| | Dosage |
| CYP2U1 | Spastic paraplegia 56, autosomal recessive |
| CYP4F22 | Autosomal recessive congenital ichthyosis 5 |
| CZ1P-ASNS, | Asparagine synthetase deficiency |
| ASNS | |
| DBH | Orthostatic hypotension 1 |
| DBT | Maple syrup urine disease, Maple syrup urine disease type 2 |
| DCAF17 | Hypogonadism, alopecia, diabetes mellitus, mental retardation and |
| | electrocardiographic abnormalities |
| DCLRE1C | Severe combined immunodeficiency, Severe combined immunodeficiency due to |
| | DCLRE1C deficiency, partial |
| DCN | Congenital Stromal Corneal Dystrophy |
| DDHD1 | Spastic paraplegia 28, autosomal recessive |
| DDRGK1 | Shohat type, Spondyloepimetaphyseal dysplasia |
| DDX3X | Delayed speech and language development, Global developmental delay, History |
| | of neurodevelopmental disorder, Mental retardation, Microcephaly, X-linked 102 |
| DDX41 | Acute myeloid leukemia, Myeloproliferative/lymphoproliferative neoplasms, |
| | familial (multiple types), susceptibility to |
| DEPDC5 | DEPDC5-Related Disorder, Familial focal epilepsy with variable foci |
| DES | Muscular dystrophy, Myofibrillar myopathy 1, Neuromuscular disease, Primary |
| | dilated cardiomyopathy, limb-girdle, type 2R |
| DGKE | Nephrotic syndrome, type 7 |
| DGUOK | Mitochondrial DNA depletion syndrome, Mitochondrial DNA-depletion syndrome |
| | 3, Progressive external ophthalmoplegia with mitochondrial DNA deletions, |
| | autosomal recessive 4, hepatocerebral, hepatocerebral form due to DGUOK |
| | deficiency |
| DHCR7 | 2-3 toe syndactyly, Congenital microcephaly, Elevated 7-dehydrocholesterol, |
| | History of neurodevelopmental disorder, Inborn genetic diseases, Small for |
| | gestational age, Smith-Lemli-Opitz syndrome |
| DHH | 46, XY sex reversal, type 7 |
| DHTKD1 | 2-aminoadipic 2-oxoadipic aciduria |
| DIAPH1 | Seizures, and microcephaly syndrome, cortical blindness |
| DICER1 | DICER1-related pleuropulmonary blastoma cancer predisposition syndrome, |
| | Hereditary cancer-predisposing syndrome |
| DIPK1A, RPL5 | Diamond-Blackfan anemia 6 |
| DLD | Maple syrup urine disease, type 3 |
| DLG3 | X-Linked mental retardation 90 |
| DLL3, PLEKHG2 | Leukodystrophy and acquired microcephaly with or without dystonia, |
| | Spondylocostal dysostosis 1, autosomal recessive |
| DLX3 | Amelogenesis imperfecta, Tricho-dento-osseous syndrome, type IV |
| DLX4 | Orofacial cleft 15 |
| DMD | Becker muscular dystrophy, Duchenne muscular dystrophy |
| DMP1 | Autosomal recessive hypophosphatemic vitamin D refractory rickets |
| DNAAF2 | Primary ciliary dyskinesia |
| DNAAF4, | Primary ciliary dyskinesia |
| DNAAF4-CCPG1 | |
| DNAH1 | Non-syndromic male infertility due to sperm motility disorder, |
| | SPERMATOGENIC FAILURE 18 |
| DNAH11 | 7, Ciliary dyskinesia, Primary ciliary dyskinesia, primary |
| DNAH17 | SPERMATOGENIC FAILURE 39 |
| DNAH5 | 3, Ciliary dyskinesia, Primary ciliary dyskinesia, primary |
| DNAI1 | Kartagener syndrome, Primary ciliary dyskinesia |
| DNAI2 | 9, Ciliary dyskinesia, Primary ciliary dyskinesia, primary |
| DNAJB2 | 5, Charcot-Marie-Tooth disease, Spinal muscular atrophy, autosomal recessive, |
| | distal |
| DNAJC12 | Hyperphenylalaninemia, mild, non-bh4-deficient |
| DNAL1 | 16, Ciliary dyskinesia, Primary ciliary dyskinesia, primary |
| DNM2 | Charcot-Marie-Tooth disease, dominant intermediate B |
| DNMBP | CATARACT 48 |
| DOCK6 | Adams-Oliver syndrome 2 |
| DOCK6, | Adams-Oliver syndrome, Adams-Oliver syndrome 2 |
| LOC105372273 | |
| DOCK8 | Hyperimmunoglobulin E recurrent infection syndrome, Inborn genetic diseases, |
| | autosomal recessive |
| DOK7 | Congenital myasthenic syndrome, Inborn genetic diseases, Myasthenia, Pena- |
| | Shokeir syndrome type I, familial, limb-girdle |
| DOLK | Congenital disorder of glycosylation type 1M |
| DONSON | AND LIMB ABNORMALITIES, MICROCEPHALY, Microcephaly-micromelia |
| | syndrome, SHORT STATURE |
| DPY19L2 | Spermatogenic failure 9 |
| DPYD | Dihydropyrimidine dehydrogenase deficiency, fluorouracil response - Other |
| DRAM2 | Cone-rod dystrophy 21, Retinal dystrophy |
| DRC1 | 21, Ciliary dyskinesia, Kartagener syndrome, Primary ciliary dyskinesia, primary |
| DSC2 | 11, Arrhythmogenic right ventricular cardiomyopathy, Arrhythmogenic right |
| | ventricular dysplasia, familial, type 11, with mild palmoplantar keratoderma and |
| | woolly hair |
| DSC2, DSCAS | Arrhythmogenic right ventricular cardiomyopathy, type 11 |
| DSG1 | Palmoplantar keratoderma i, focal, or diffuse, striate |
| DSG1, DSG1- | Erythroderma, and hyper-ige, congenital, hypotrichosis, with palmoplantar |
| AS1 | keratoderma |
| DSG2 | Arrhythmogenic right ventricular cardiomyopathy, Cardiac arrest, |
| | Cardiomyopathy, Cardiovascular phenotype, Dilated Cardiomyopathy, Dominant, |
| | Hypertrophic cardiomyopathy, type 10 |
| DSG2, DSG2- | Dilated cardiomyopathy 1BB |
| AS1 | |
| DSG4, DSG1- | Hypotrichosis 6 |
| AS1 | |
| DSP | Arrhythmogenic right ventricular cardiomyopathy, Arrhythmogenic right |
| | ventricular dysplasia/cardiomyopathy, Cardiac arrest, Cardiomyopathy, |
| | Cardiovascular phenotype, DSP-Related Disorders, Dilated cardiomyopathy with |
| | woolly hair and keratoderma, Keratosis palmoplantaris striata II, Left ventricular |
| | noncompaction cardiomyopathy, Lethal acantholytic epidermolysis bullosa, Long |
| | QT syndrome 1, Primary dilated cardiomyopathy, Skin fragility-woolly hair- |
| | palmoplantar keratoderma syndrome, Ventricular tachycardia, and tooth agenesis, |
| | dilated, keratoderma, type 8, with woolly hair |
| DST | Epidermolysis bullosa simplex, Neuropathy, autosomal recessive 2, hereditary |
| | sensory and autonomic, type VI |
| DUOX2 | Congenital hypothyroidism, Familial thyroid dyshormonogenesis, Inborn genetic |
| | diseases, Nongoitrous Euthyroid Hyperthyrotropinemia, Thyroid |
| | dyshormonogenesis 6 |
| DVL3 | Robinow syndrome, autosomal dominant 1, autosomal dominant 3 |
| DYNC2H1 | Jeune thoracic dystrophy, Short Rib Polydactyly Syndrome, Short-rib polydactyly |
| | syndrome type III, Short-rib thoracic dysplasia 3 with or without polydactyly |
| DYNC2I1 | Short-rib thoracic dysplasia 8 with or without polydactyly |
| DYNC2I2 | Jeune thoracic dystrophy, Short-rib thoracic dysplasia 11 with or without |
| | polydactyly |
| DYNC2LI1 | Short-rib thoracic dysplasia 15 with polydactyly |
| DYRK1A | Mental retardation, autosomal dominant 7 |
| DYSF | Autosomal recessive limb-girdle muscular dystrophy type 2B, Miyoshi muscular |
| | dystrophy 1, Myopathy, Qualitative or quantitative defects of dysferlin, distal, with |
| | anterior tibial onset |
| ECEL1 | Distal arthrogryposis type 5D, Inborn genetic diseases |
| ECHS1 | Inborn genetic diseases, Mitochondrial short-chain enoyl-coa hydratase 1 |
| | deficiency |
| ECM1 | Lipid proteinosis |
| EDA | Hypohidrotic X-linked ectodermal dysplasia |
| EDARADD | Ectodermal dysplasia 11b, autosomal recessive, hypohidrotic/hair/tooth type |
| EDN3 | Congenital central hypoventilation, Dominant, Hirschsprung Disease, |
| | Hirschsprung disease, Waardenburg syndrome, Waardenburg syndrome type 4B |
| EDNRB, | Rare genetic deafness |
| EDNRB-AS1 | |
| EFEMP2 | Autosomal recessive cutis laxa type 1B, Autosomal recessive cutis laxa type IA |
| EHMT1 | Kleefstra syndrome 1 |
| EIF2AK3 | Wolcott-Rallison dysplasia |
| EIF2AK4 | Pulmonary venoocclusive disease 2, autosomal recessive |
| EIF2B2 | Leukoencephalopathy with vanishing white matter, Ovarioleukodystrophy |
| EIF2S3 | MEHMO syndrome |
| ELN | Inborn genetic diseases, Supravalvar aortic stenosis |
| ELOVL4 | Retinal dystrophy, Stargardt Disease 3 |
| ELP1 | Familial dysautonomia |
| ELP2 | ELP2-Related Disorders, Mental retardation, autosomal recessive 58 |
| EMD | Cardiovascular phenotype, Emery-Dreifuss muscular dystrophy 1, Neuromuscular |
| | disease, X-linked |
| ENAM | Amelogenesis imperfecta, Amelogenesis imperfecta - hypoplastic autosomal |
| | dominant - local, type IC |
| ENG | Hereditary hemorrhagic telangiectasia, Hereditary hemorrhagic telangiectasia type |
| | 1 |
| ENG, | Hereditary hemorrhagic telangiectasia type 1 |
| LOC102723566 | |
| EOGT | Adams-Oliver syndrome, Adams-Oliver syndrome 4 |
| EPB42 | Spherocytosis type 5 |
| EPCAM | Diarrhea 5, congenital, with tufting enteropathy |
| EPG5 | Vici syndrome |
| EPHB4 | Capillary malformation-arteriovenous malformation 2 |
| EPHB4, | Capillary malformation-arteriovenous malformation 2 |
| SLC12A9 | |
| EPOR | Primary familial polycythemia due to EPO receptor mutation |
| ERCC2 | Metachromatic leukodystrophy variant, Trichothiodystrophy 1, Xeroderma |
| | pigmentosum, group D, photosensitive |
| ERCC3 | Xeroderma pigmentosum, complementation group b |
| ERCC4 | Cockayne syndrome, Fanconi anemia, Hutchinson-Gilford syndrome, Pre-B-cell |
| | acute lymphoblastic leukemia, XFE progeroid syndrome, Xeroderma |
| | pigmentosum, complementation group Q, group F |
| ERCC5, BIVM- | Xeroderma pigmentosum, group G |
| ERCC5 | |
| ERCC6 | Cerebrooculofacioskeletal syndrome 1, Cockayne syndrome B, DE SANCTIS- |
| | CACCHIONE SYNDROME |
| ERCC8 | Cockayne syndrome type A |
| ERCC8, ERCC8- | Cockayne syndrome type A |
| AS1 | |
| ERCC8, | Cockayne syndrome type A, MITOCHONDRIAL COMPLEX I DEFICIENCY, |
| NDUFAF2 | NUCLEAR TYPE 10 |
| ERF | Craniosynostosis 1, Craniosynostosis 4 |
| ERI1 | Abnormality of finger, Coarse facial features, Global developmental delay, |
| | Unilateral renal agenesis |
| ESCO2 | Roberts-SC phocomelia syndrome |
| ESRP1 | AUTOSOMAL RECESSIVE 109, DEAFNESS |
| ESRRB | Rare genetic deafness |
| ETFDH | Multiple acyl-CoA dehydrogenase deficiency |
| ETHE1 | Ethylmalonic encephalopathy |
| EVC2 | Curry-Hall syndrome, Ellis-van Creveld syndrome |
| EXOSC3 | Pontocerebellar hypoplasia, type 1b |
| EXPH5 | Epidermolysis bullosa, autosomal recessive, nonspecific |
| EXT1 | Chondrosarcoma, Multiple congenital exostosis, Multiple exostoses type 1, |
| | sporadic |
| EXT2 | Multiple exostoses type 2 |
| EYA1 | Branchiootic syndrome, Melnick-Fraser syndrome, Rare genetic deafness |
| EYA4 | Deafness, Dilated cardiomyopathy 1J, Rare genetic deafness, autosomal dominant |
| | 10 |
| EYA4, TARID | EYA4-Related Disorders |
| EYS | Retinal dystrophy, Retinitis pigmentosa, Retinitis pigmentosa 25 |
| F13A1 | Factor XIII subunit A deficiency |
| F13B | Factor XIII, b subunit, deficiency of |
| F2 | Prothrombin deficiency, congenital |
| F5 | Factor V deficiency |
| F8 | Hereditary factor VIII deficiency disease |
| F9 | Hereditary factor IX deficiency disease, Thrombophilia, X-linked, due to factor IX |
| | defect |
| FA2H | Spastic paraplegia 35 |
| FAH | Tyrosinemia type I |
| FAM161A | Retinal dystrophy, Retinitis pigmentosa, Retinitis pigmentosa 28 |
| FAM20A | Amelogenesis imperfecta type 1G |
| FANCA | Fanconi anemia, complementation group A |
| FANCB | Fanconi anemia, complementation group B |
| FANCC | Fanconi anemia, Hereditary cancer-predisposing syndrome, complementation |
| | group C |
| FANCC, AOPEP | Fanconi anemia, Hereditary cancer-predisposing syndrome, Tracheoesophageal |
| | fistula, complementation group C |
| FANCF | Fanconi anemia, complementation group F |
| FANCM | Fanconi anemia, Malignant germ cell tumor of ovary, SPERMATOGENIC |
| | FAILURE 28 |
| FARS2 | Combined oxidative phosphorylation deficiency 14 |
| FARSB | Interstitial lung and liver disease, Rajab interstitial lung disease with brain |
| | calcifications |
| FAS | Autoimmune lymphoproliferative syndrome |
| FAT4 | Van Maldergem syndrome |
| FBN1 | Acromicric dysplasia, Acute aortic dissection, Cardiovascular phenotype, Ectopia |
| | lentis, Familial thoracic aortic aneurysm, Familial thoracic aortic aneurysm and |
| | aortic dissection, Geleophysic dysplasia 2, Inborn genetic diseases, MASS |
| | syndrome, Marfan Syndrome/Loeys-Dietz Syndrome/Familial Thoracic Aortic |
| | Aneurysms and Dissections, Marfan lipodystrophy syndrome, Marfan syndrome, |
| | Stiff skin syndrome, Weill-Marchesani syndrome 2, autosomal dominant, isolated |
| FBN1, | Marfan Syndrome/Loeys-Dietz Syndrome/Familial Thoracic Aortic Aneurysms |
| LOC113939944 | and Dissections, Marfan syndrome |
| FBXL4 | Inborn genetic diseases, Mitochondrial DNA depletion syndrome, Mitochondrial |
| | DNA depletion syndrome 13 (encephalomyopathic type) |
| FERMT1 | Kindlers syndrome |
| FEZF1-AS1, | Hypogonadotropic hypogonadism 22 with anosmia |
| FEZF1 | |
| FGD4 | Charcot-Marie-Tooth disease, Charcot-Marie-Tooth disease type 4 |
| FGF16 | Metacarpal 4-5 fusion |
| FGF3 | Deafness with labyrinthine aplasia microtia and microdontia (LAMM) |
| FGG | Afibrinogenemia, Hypofibrinogenemia, congenital |
| FH | Fumarase deficiency, Hereditary cancer-predisposing syndrome, Hereditary |
| | leiomyomatosis and renal cell cancer |
| FIG4 | Charcot-Marie-Tooth disease, Charcot-Marie-Tooth disease type 4, Yunis-Varon |
| | syndrome, type 4J |
| FKBP10 | Bruck syndrome 1, Osteogenesis imperfecta type 12 |
| FKBP14, | Congenital muscular dystrophy, Ehlers-Danlos syndrome with progressive |
| FKBP14-AS1 | kyphoscoliosis, Inborn genetic diseases, Joint hypermobility, Muscular hypotonia, |
| | Pes valgus, Thoracolumbar scoliosis, and hearing loss, myopathy |
| FKRP | Limb-girdle muscular dystrophy-dystroglycanopathy, type C5 |
| FKTN | Congenital muscular dystrophy-dystroglycanopathy with brain and eye anomalies, |
| | Congenital muscular dystrophy-dystroglycanopathy without mental retardation, |
| | FKTN-Related Disorders, Fukuyama congenital muscular dystrophy, Limb-girdle |
| | muscular dystrophy-dystroglycanopathy, Walker-Warburg congenital muscular |
| | dystrophy, type A4, type B4, type C4 |
| FLCN | Hereditary cancer-predisposing syndrome, Multiple fibrofolliculomas, |
| | Pneumothorax, primary spontaneous |
| FLG | 2, Dermatitis, FLG-Related Disorder, Ichthyosis vulgaris, atopic, susceptibility to |
| FLNA | Periventricular nodular heterotopia 1 |
| FLNB | Spondylocarpotarsal synostosis syndrome |
| FLNC | 26, 4, Cardiomyopathy, Dilated Cardiomyopathy, Dominant, Myofibrillar |
| | myopathy, Myopathy, distal, familial hypertrophic, filamin C-related |
| FLNC, FLNC- | 26, 4, Cardiomyopathy, Dilated Cardiomyopathy, Dominant, Myofibrillar |
| AS1 | myopathy, Myopathy, distal, familial hypertrophic, filamin C-related |
| FLT4 | 7, CONGENITAL HEART DEFECTS, MULTIPLE TYPES |
| FMR1 | Intellectual disability |
| FOXF1 | Persistent fetal circulation syndrome |
| FOXG1 | History of neurodevelopmental disorder, Rett syndrome, congenital variant |
| FOXL2 | Blepharophimosis, and epicanthus inversus, and epicanthus inversus syndrome |
| | type 1, ptosis |
| FOXN1 | AUTOSOMAL DOMINANT, INFANTILE, T-CELL LYMPHOPENIA, T-cell |
| | immunodeficiency, WITH OR WITHOUT NAIL DYSTROPHY, and nail |
| | dystrophy, congenital alopecia |
| FOXP1 | Mental retardation with language impairment and with or without autistic features |
| FOXRED1 | Leigh syndrome, Mitochondrial complex I deficiency, nuclear type 1 |
| FRAS1 | Fraser syndrome 1 |
| FREM2 | Cryptophthalmos, FRASER SYNDROME 2, Fraser syndrome 1, isolated, |
| | unilateral or bilateral |
| FSHB | Hypogonadotropic hypogonadism 24 without anosmia |
| FSIP2 | SPERMATOGENIC FAILURE 34 |
| FSIP2, FSIP2- | SPERMATOGENIC FAILURE 34 |
| AS1 | |
| FTCD | GLUTAMATE FORMIMINOTRANSFERASE DEFICIENCY |
| FTSJ1 | Mental retardation 9, X-linked |
| FUCA1 | Fucosidosis |
| FYCO1 | Cataract 18 |
| FZD4, PRSS23 | Exudative retinopathy, Familial exudative vitreoretinopathy |
| G6PC | Glycogen storage disease, Glycogen storage disease due to glucose-6-phosphatase |
| | deficiency type IA |
| GAA | Glycogen storage disease, type II |
| GABRA1 | 19, Epilepsy, Epileptic encephalopathy, early infantile, juvenile myoclonic 5 |
| GABRA6 | GABRA6-Related Disorder |
| GALC | Galactosylceramide beta-galactosidase deficiency |
| GALM | GALACTOSEMIA IV |
| GALNS | MPS-IV-A, Morquio syndrome, Mucopolysaccharidosis |
| GALT | Deficiency of UDPglucose-hexose-1-phosphate uridylyltransferase |
| GAMT | Cerebral creatine deficiency syndrome, Deficiency of guanidinoacetate |
| | methyltransferase |
| GAREM2, | Long-chain 3-hydroxyacyl-CoA dehydrogenase deficiency, Mitochondrial |
| HADHA | trifunctional protein deficiency |
| GATA1 | Acute megakaryoblastic leukemia |
| GATA3 | Hypoparathyroidism-deafness-renal disease syndrome |
| GATA6 | Abnormality of cardiovascular system morphology, Congenital diaphragmatic |
| | hernia, Pancreatic agenesis and congenital heart disease, Persistent truncus |
| | arteriosus |
| GATAD1, PEX1 | Deafness enamel hypoplasia nail defects, Peroxisome biogenesis disorder 1A |
| | (Zellweger) |
| GATAD2B | GATAD2B-Related Disorder, Mental retardation, autosomal dominant 18 |
| GBA | Acute neuronopathic Gauchers disease, Gaucher disease, Gaucher disease type |
| | 3C, Gauchers disease, Subacute neuronopathic Gauchers disease, type 1 |
| GBA, | Gaucher disease, Gauchers disease, perinatal lethal, type 1 |
| LOC106627981 | |
| GBE1 | Glycogen storage disease, Glycogen storage disease IV, classic hepatic, fatal |
| | perinatal neuromuscular, type IV |
| GCDH | Glutaric aciduria, type 1 |
| GCH1 | Dystonia 5 |
| GCK | Maturity onset diabetes mellitus in young, Maturity-onset diabetes of the young, |
| | type 2 |
| GDAP1 | Charcot-Marie-Tooth disease, recessive intermediate A, type 4A |
| GDF1, CERS1 | Heterotaxia |
| GDF9 | PREMATURE OVARIAN FAILURE 14 |
| GFER | Mitochondrial diseases |
| GHR | Laron syndrome with elevated serum GH-binding protein, Laron-type isolated |
| | somatotropin defect |
| GJB1 | Charcot-Marie-Tooth Neuropathy X, Charcot-Marie-Tooth disease |
| GJB2 | Bilateral conductive hearing impairment, Bilateral sensorineural hearing |
| | impairment, Deafness, Dominant, GJB2-Related Disorders, GJB2/GJB3, |
| | GJB2/GJB6, Hearing impairment, Hearing loss, Hystrix-like ichthyosis with |
| | deafness, Inborn genetic diseases, Keratitis ichthyosis and deafness syndrome, |
| | Keratitis-ichthyosis-deafness syndrome, Knuckle pads, Mutilating keratoderma, |
| | Nonsyndromic Hearing Loss, Nonsyndromic hearing loss and deafness, |
| | Palmoplantar keratoderma-deafness syndrome, Rare genetic deafness, Recessive, |
| | Severe sensorineural hearing impairment, X-linked 2, autosomal dominant, |
| | autosomal dominant 3a, autosomal recessive 1A, autosomal recessive 1b, deafness |
| | AND leukonychia syndrome, digenic |
| GJB3 | Deafness, autosomal dominant 2b |
| GLA, RPL36A- | Fabry disease |
| HNRNPH2 | |
| GLB1 | GLB1-Related Disorders, GM1 gangliosidosis, GM1 gangliosidosis type 2, GM1 |
| | gangliosidosis type 3, GM1-gangliosidosis, Infantile GM1 gangliosidosis, MPS- |
| | IV-B, Mucopolysaccharidosis, type I, with cardiac involvement |
| GLDC | Non-ketotic hyperglycinemia |
| GLDN | Lethal congenital contracture syndrome 11 |
| GLI3 | Greig cephalopolysyndactyly syndrome, Pallister-Hall syndrome, Postaxial |
| | polydactyly, Preaxial polydactyly 4, type A1/B |
| GLIS3 | Diabetes mellitus, neonatal, with congenital hypothyroidism |
| GLMN | Glomuvenous malformations |
| GLRA1 | Hyperekplexia 1 |
| GNAS | Progressive osseous heteroplasia, Pseudohypoparathyroidism, |
| | Pseudopseudohypoparathyroidism |
| GNAT2 | Achromatopsia 4 |
| GNB5 | Intellectual developmental disorder with cardiac arrhythmia, Language delay and |
| | attention deficit-hyperactivity disorder/cognitive impairment with or without |
| | cardiac arrhythmia |
| GNPAT | Rhizomelic chondrodysplasia punctata type 2 |
| GNPTAB | GNPTAB-Related Disorders, Inborn genetic diseases, MUCOLIPIDOSIS III |
| | ALPHA/BETA, Mucolipidosis, Mucolipidosis type II, Pseudo-Hurler |
| | polydystrophy |
| GNPTG | Mucolipidosis, Mucolipidosis type III gamma |
| GORAB | Geroderma osteodysplastica |
| GOSR2, | Progressive myoclonic epilepsy |
| LRRC37A2 | |
| GPC3 | Simpson-Golabi-Behmel syndrome, Wilms tumor 1 |
| GPC4 | Keipert syndrome |
| GPC6 | Autosomal recessive omodysplasia |
| GPC6, GPC6-AS2 | Autosomal recessive omodysplasia |
| GPI | Hemolytic anemia, due to glucose phosphate isomerase deficiency, nonspherocytic |
| GPNMB | 3, AMYLOIDOSIS, PRIMARY LOCALIZED CUTANEOUS |
| GPR143 | Ocular albinism, type I |
| GPR179 | Congenital stationary night blindness, Retinal dystrophy, type 1E |
| GPSM2 | Chudley-McCullough syndrome, GPSM2-Related Disorders, Rare genetic |
| | deafness |
| GRHL2 | Deafness, autosomal dominant 28 |
| GRHL3 | Van der Woude syndrome 2 |
| GRHPR | Nephrocalcinosis, Nephrolithiasis, Primary hyperoxaluria, type II |
| GRIN2B | Mental retardation, autosomal dominant 6 |
| GRIP1 | FRASER SYNDROME 3 |
| GRN | Frontotemporal dementia |
| GRXCR1 | Deafness, Rare genetic deafness, autosomal recessive 25 |
| GSDME | Deafness, autosomal dominant 5 |
| GUCY2C, | Meconium ileus |
| C12orf60 | |
| GUSB | Mucopolysaccharidosis type 6, Mucopolysaccharidosis type 7 |
| GYG1 | Glycogen storage disease XV, Polyglucosan body myopathy 2 |
| GYS1 | Glycogen storage disease 0, muscle |
| GYS2 | Glycogen storage disease, Glycogen storage disease due to hepatic glycogen |
| | synthase deficiency |
| GZF1 | AND MYOPIA, JOINT LAXITY, SHORT STATURE |
| H1-4 | Inborn genetic diseases, RAHMAN SYNDROME |
| H6PD | Cortisone reductase deficiency 1 |
| HADHA | HADHA-Related Disorders, Long-chain 3-hydroxyacyl-CoA dehydrogenase |
| | deficiency, Mitochondrial trifunctional protein deficiency |
| HADHA, | HADHA-Related Disorders, Inborn genetic diseases, LCHAD Deficiency, Lchad |
| GAREM2 | deficiency with maternal acute fatty liver of pregnancy, Long-chain 3- |
| | hydroxyacyl-CoA dehydrogenase deficiency, Mitochondrial trifunctional protein |
| | deficiency |
| HAX1 | Severe congenital neutropenia 3, autosomal recessive |
| HBA2, | Alpha plus thalassemia |
| LOC106804612 | |
| HBB, | Anemia, Beta thalassemia major, Beta-plus-thalassemia, Beta-thalassemia, |
| LOC106099062, | Erythrocytosis 6, Fetal hemoglobin quantitative trait locus 1, HBB-Related |
| LOC107133510 | Disorders, Hb SS disease, Heinz body anemia, Hemoglobin E, Hemoglobin E |
| | disease, Hemoglobin E/beta thalassemia disease, Hemoglobin M disease, |
| | Hemoglobinopathy, Malaria, Susceptibility to malaria, alpha Thalassemia, beta |
| | Thalassemia, beta\hat{ }0\hat{ } Thalassemia, dominant inclusion body type, familial, |
| | resistance to |
| HBB, | beta Thalassemia |
| LOC107133510, | |
| LOC110006319 | |
| HCN4 | Brugada syndrome 8, Sick sinus syndrome 2, autosomal dominant |
| HEXA | Inborn genetic diseases, Tay-Sachs disease |
| HEXB | Sandhoff disease, infantile |
| HFM1 | Premature ovarian failure 9 |
| HGD | Alkaptonuria |
| HGSNAT | MPS-III-C, Mucopolysaccharidosis, Retinitis pigmentosa 73, Sanfilippo syndrome |
| HIVEP2 | Angelman syndrome-like, Mental retardation, autosomal dominant 43 |
| HJV | Hemochromatosis type 2A |
| HLCS | Holocarboxylase synthetase deficiency |
| HMCN1 | Age-related macular degeneration 1 |
| HMGB3 | Microphthalmia, syndromic 13 |
| HMGCL | Deficiency of hydroxymethylglutaryl-CoA lyase |
| HNF1A | 20, Clear cell carcinoma of kidney, Diabetes mellitus, Diabetes mellitus type 1, |
| | Hepatic adenomas, Maturity onset diabetes mellitus in young, Maturity-onset |
| | diabetes of the young, familial, insulin-dependent, type 3 |
| HNF1B | Familial hypoplastic, Renal cysts and diabetes syndrome, glomerulocystic kidney |
| HNRNPK | AU-KLINE SYNDROME |
| HNRNPU | Epileptic encephalopathy |
| HOXA1 | Athabaskan brainstem dysgenesis syndrome, Bosley-Salih-Alorainy syndrome |
| HOXA11 | Radioulnar synostosis with amegakaryocytic thrombocytopenia 1 |
| HOXD13 | Synpolydactyly 1 |
| HPGD | 1, HPGD-Related Disorders, Hypertrophic osteoarthropathy, autosomal recessive, |
| | primary |
| HPS1 | Hermansky-Pudlak syndrome, Hermansky-Pudlak syndrome 1 |
| HPS5 | Hermansky-Pudlak syndrome, Hermansky-Pudlak syndrome 5 |
| HPS6 | Hermansky-Pudlak syndrome, Hermansky-Pudlak syndrome 6 |
| HPSE2 | Urofacial syndrome 1 |
| HR | Atrichia with papular lesions |
| HSD17B10 | HSD10 disease |
| HSD17B4 | Bifunctional peroxisomal enzyme deficiency, Perrault syndrome |
| HSPA9 | 4, Anemia, Even-plus syndrome, sideroblastic |
| HSPB1 | Charcot-Marie-Tooth disease, Charcot-Marie-Tooth disease axonal type 2F, Distal |
| | hereditary motor neuronopathy type 2B |
| HSPG2 | Lethal Kniest-like syndrome, Schwartz-Jampel syndrome |
| HYAL1 | Deficiency of hyaluronoglucosaminidase |
| HYDIN | 5, Ciliary dyskinesia, primary |
| ICAM4 | Landsteiner-Wiener phenotype |
| IDS | MPS-II, Mucopolysaccharidosis |
| IDS, | MPS-II, Mucopolysaccharidosis |
| LOC106050102 | |
| IDUA | Hurler syndrome, MPS-I-H/S, MPS-I-S, Mucopolysaccharidosis, |
| | Mucopolysaccharidosis type 1 |
| IDUA, SLC26A1 | Hurler syndrome, MPS-I-H/S, MPS-I-S, Mucopolysaccharidosis, |
| | Mucopolysaccharidosis type 1 |
| IFIH1 | Aicardi-Goutieres syndrome 7, Singleton-Merten syndrome 1 |
| IFNGR1 | Disseminated atypical mycobacterial infection, IFN-gamma receptor 1 deficiency, |
| | Immunodeficiency 27b, Inherited Immunodeficiency Diseases |
| IFNGR2 | Immunodeficiency 28 |
| IFT140 | Retinitis pigmentosa 80 |
| IFT140, | Jeune thoracic dystrophy, Joubert syndrome with Jeune asphyxiating thoracic |
| LOC105371046 | dystrophy, Renal dysplasia, cerebellar ataxia and skeletal dysplasia, retinal |
| | pigmentary dystrophy |
| IFT172 | Short-rib thoracic dysplasia 10 with or without polydactyly |
| IFT52 | Short Rib Polydactyly Syndrome, Short-rib thoracic dysplasia 16 with or without |
| | polydactyly |
| IGF1 | Growth delay due to insulin-like growth factor type 1 deficiency |
| IGF1R | Inborn genetic diseases |
| IGFALS | Acid-labile subunit deficiency |
| IGHM | Agammaglobulinemia, non-Bruton type |
| IGHMBP2 | 1, Autosomal dominant distal hereditary motor neuropathy, Charcot-Marie-Tooth |
| | disease, Distal spinal muscular atrophy, Inborn genetic diseases, Spinal muscular |
| | atrophy, autosomal recessive, axonal, distal, type 2S |
| IGLL1 | Agammaglobulinemia 2, autosomal recessive |
| IGSF1 | Hypothyroidism, and testicular enlargement, central |
| IGSF3 | Lacrimal duct defect |
| IKBKG | Ectodermal dysplasia and immunodeficiency 1, Immunodeficiency without |
| | anhidrotic ectodermal dysplasia, Incontinentia pigmenti, atypical |
| IL12B | Immunodeficiency 29 |
| IL12RB1 | Immunodeficiency 30 |
| IL2RB | Ichthyosis (disease) |
| IL2RG | Combined immunodeficiency, X-linked, X-linked severe combined |
| | immunodeficiency |
| IL36RN | Pustular psoriasis, generalized |
| IL7R | B cell-positive, NK cell-positive, Severe combined immunodeficiency, T cell- |
| | negative, autosomal recessive |
| INPP5E | Retinal dystrophy |
| INPPL1 | Opsismodysplasia |
| INTU | Mohr syndrome, Orofaciodigital syndrome 17 |
| IQCB1 | Renal dysplasia and retinal aplasia |
| IQCE | POLYDACTYLY, POSTAXIAL, TYPE A7 |
| IQSEC2 | Mental retardation, Severe intellectual deficiency, X-linked 1 |
| IRAK4 | Immunodeficiency due to interleukin-1 receptor-associated kinase-4 deficiency |
| IRF2BPL | ABNORMAL MOVEMENTS, AND SEIZURES, LOSS OF SPEECH, |
| | NEURODEVELOPMENTAL DISORDER WITH REGRESSION, |
| | Neurodevelopmental disorder |
| IRF6 | Van der Woude syndrome |
| IRS4 | 9, CONGENITAL, HYPOTHYROIDISM, NONGOITROUS |
| ISCA2 | Multiple mitochondrial dysfunctions syndrome 4 |
| ISG15 | Immunodeficiency 38 with basal ganglia calcification |
| ITGA7 | Muscular dystrophy, congenital, due to integrin alpha-7 deficiency |
| ITGB2 | Leukocyte adhesion deficiency |
| ITGB4 | Epidermolysis bullosa junctionalis with pyloric atresia |
| ITPA | 35, Epileptic encephalopathy, Inosine triphosphatase deficiency, early infantile |
| ITPR1 | Gillespie syndrome |
| IVD | Isovaleric acidemia, Isovaleryl-CoA dehydrogenase deficiency, type III |
| JAG1 | Alagille syndrome 1, Arteriohepatic dysplasia, Heart, malformation of |
| JAK3 | B cell-positive, NK cell-negative, Severe combined immunodeficiency, Severe |
| | combined immunodeficiency disease, T cell-negative, autosomal recessive |
| KAT6A | History of neurodevelopmental disorder, Mental retardation, autosomal dominant |
| | 32 |
| KAT6B | Blepharophimosis - intellectual disability syndrome, SBBYS type |
| KAT6B, DUPD1 | Blepharophimosis - intellectual disability syndrome, Genitopatellar syndrome, |
| | Inborn genetic diseases, SBBYS type |
| KATNIP | Joubert syndrome 26 |
| KCNA1 | Episodic ataxia type 1 |
| KCNA5 | 7, Atrial fibrillation, familial |
| KCNC1 | Epilepsy, progressive myoclonic 7 |
| KCNE1 | Long QT syndrome |
| KCNH2 | Cardiac arrhythmia, Cardiovascular phenotype, Congenital long QT syndrome, |
| | Long QT syndrome, Long QT syndrome 1/2, Long QT syndrome 2, digenic |
| KCNK18 | Migraine, with or without aura 13 |
| KCNQ1 | Cardiac arrhythmia, Cardiovascular phenotype, Congenital long QT syndrome, |
| | Jervell and Lange-Nielsen syndrome, Jervell and Lange-Nielsen syndrome 1, |
| | KCNQ1-Related Disorders, LQT1 subtype, Long QT syndrome, Long QT |
| | syndrome 1, Rare genetic deafness, Romano-Ward syndrome, recessive |
| KCNQ1-AS1, | Jervell and Lange-Nielsen syndrome 1 |
| KCNQ1 | |
| KCNQ1, KCNQ1- | Cardiovascular phenotype, Long QT syndrome, Long QT syndrome 1 |
| AS1 | |
| KCNQ1, | Congenital long QT syndrome, LQT1 subtype, Long QT syndrome |
| KCNQ1OT1 | |
| KCNQ2 | Benign familial neonatal seizures 1, Early infantile epileptic encephalopathy, Early |
| | infantile epileptic encephalopathy 7, Epileptic encephalopathy, Inborn genetic |
| | diseases, Seizures |
| KCNQ3 | Intellectual disability, Seizures |
| KCNQ4 | Autosomal dominant nonsyndromic deafness 2A |
| KCNT1 | 5, Early infantile epileptic encephalopathy 14, Epilepsy, nocturnal frontal lobe |
| KCNV2 | Cone dystrophy with supernormal rod response, Progressive cone dystrophy |
| | (without rod involvement), Retinal dystrophy, Stargardt disease |
| KDM5B | Intellectual disability, autosomal recessive 65 |
| KDM5C | Claes-Jensen type, Mental retardation, X-linked, syndromic |
| KDM6A | Kabuki syndrome 2 |
| KERA | Cornea plana 2 |
| KHDC3L | 2, Hydatidiform mole, recurrent |
| KIAA0586 | Congenital cerebellar hypoplasia, Intellectual disability, Joubert syndrome, Joubert |
| | syndrome 23, Retinal dystrophy, Rod-cone dystrophy, Short-rib thoracic dysplasia |
| | 14 with polydactyly |
| KIAA0753 | Orofaciodigital syndrome XV |
| KIAA0825 | POLYDACTYLY, POSTAXIAL, Postaxial polydactyly type A1, TYPE A10 |
| KIΛA1549 | RETINITIS PIGMENTOSA 86 |
| KIF11 | Microcephaly with or without chorioretinopathy, lymphedema, or mental |
| | retardation |
| KIF7 | Acrocallosal syndrome, Joubert syndrome 12 |
| KIFBP | Goldberg-Shprintzen megacolon syndrome |
| KISS1R | Hypogonadotropic hypogonadism 8 without anosmia |
| KIZ | Retinitis pigmentosa 69 |
| KMT2A | Wiedemann-Steiner syndrome |
| KMT2B | Dystonia 28, childhood-onset |
| KMT2C | Kleefstra syndrome due to a point mutation |
| KMT2D | CHARGE association, Kabuki syndrome, Kabuki syndrome 1 |
| KMT2E | Epilepsy, Leukoencephalopathy, Macrocephalus, O''''DONNELL-LURIA-RODAN |
| | SYNDROME, See cases, intellectual deficiency |
| KPTN | Mental retardation, autosomal recessive 41 |
| KRIT1 | Cavernous malformations of CNS and retina, Cerebral cavernous malformation, |
| | Cerebral cavernous malformations 1 |
| KRT1 | Ichthyosis histrix, curth-macklin type |
| KRT10 | Bullous ichthyosiform erythroderma |
| KRT10, TMEM99 | Bullous ichthyosiform erythroderma |
| KRT14 | Epidermolysis bullosa simplex, autosomal recessive |
| KRT5 | Dowling-Degos disease 1 |
| KRT6A | Pachyonychia congenita 3 |
| KRT85 | ‘pure’ hair-nail type, Ectodermal dysplasia |
| KYNU | AND LIMB DEFECTS SYNDROME 2, CARDIAC, Congenital NAD deficiency |
| | disorder, RENAL, VERTEBRAL |
| L1CAM | MASA syndrome, Spastic paraplegia |
| L2HGDH | L-2-hydroxyglutaric aciduria |
| LACC1 | JUVENILE ARTHRITIS |
| LAMA2 | Inborn genetic diseases, Laminin alpha 2-related dystrophy, Merosin deficient |
| | congenital muscular dystrophy |
| LAMA3 | Junctional epidermolysis bullosa gravis of Herlitz |
| LAMA4 | Dilated cardiomyopathy 1JJ |
| LAMB3 | Amelogenesis imperfecta, Junctional epidermolysis bullosa, Junctional |
| | epidermolysis bullosa gravis of Herlitz, non-Herlitz type, type IA |
| LAMC2 | Junctional epidermolysis bullosa, Junctional epidermolysis bullosa gravis of |
| | Herlitz, non-Herlitz type |
| LAMP2 | Cardiomyopathy, Danon disease, Hypertrophic cardiomyopathy, Primary dilated |
| | cardiomyopathy |
| LARGE1 | Congenital muscular dystrophy-dystroglycanopathy with mental retardation, type |
| | B6 |
| LBR | Disproportionate short stature, Femoral bowing, Pelger-HuÃ«t anomaly, |
| | Regressive spondylometaphyseal dysplasia, Retrognathia, Rhizomelic arm |
| | shortening, Rhizomelic leg shortening, Short long bone |
| LDB3 | Cardiomyopathy, Myofibrillar myopathy, ZASP-related |
| LDLR | Familial hypercholesterolemia, Familial hypercholesterolemia 1, Homozygous |
| | familial hypercholesterolemia |
| LDLRAP1 | Familial hypercholesterolemia 4 |
| LEP | Leptin deficiency or dysfunction |
| LFNG | Spondylocostal dysostosis 3, autosomal recessive |
| LGI1 | Familial temporal lobe epilepsy 1 |
| LHFPL5 | Rare genetic deafness |
| LHX3 | Non-acquired combined pituitary hormone deficiency with spine abnormalities |
| LIFR | StÃ¼ve-Wiedemann syndrome |
| LIG4 | LIG4-Related Disorders, Lig4 syndrome |
| LIPA | Lysosomal acid lipase deficiency |
| LIPE | Familial partial lipodystrophy 6 |
| LIPE, LIPE-AS1, | Familial partial lipodystrophy 6 |
| LOC101930071 | |
| LIPH | Hypotrichosis 7, Woolly hair, autosomal recessive 2, with or without hypotrichosis |
| LIPN | Autosomal recessive congenital ichthyosis 8 |
| LMBR1 | Acheiropodia |
| LMBRD1 | Inborn genetic diseases, Methylmalonic aciduria and homocystinuria type cblF |
| LMNA | Cardiovascular phenotype, Charcot-Marie-Tooth disease, Primary dilated |
| | cardiomyopathy, type 2 |
| LMOD3 | Nemaline myopathy 10 |
| LMX1B | Nail-patella syndrome |
| LOC100507346, | Gorlin syndrome, Medulloblastoma |
| PTCH1 | |
| LOC101927055, | Dilated cardiomyopathy 1G, Limb-girdle muscular dystrophy, Primary dilated |
| TTN | cardiomyopathy, type 2J |
| LOC101927157, | Retinitis pigmentosa, Retinitis pigmentosa 49 |
| CNGA1 | |
| LOC101927188, | Poretti-Boltshauser syndrome |
| LAMA1 | |
| LOC102723566, | Hereditary hemorrhagic telangiectasia type 1 |
| ENG | |
| LOC106694316, | Myeloperoxidase deficiency |
| MPO | |
| LOC110006319, | beta Thalassemia |
| HBB, | |
| LOC107133510 | |
| LOXHD1 | Deafness, Rare genetic deafness, autosomal recessive 77 |
| LPL | Hyperlipoproteinemia, Lpl-arita, type I |
| LRAT | EARLY-ONSET SEVERE, JUVENILE, LRAT-RELATED, Leber congenital |
| | amaurosis, Leber congenital amaurosis 14, RETINAL DYSTROPHY, RETINITIS |
| | PIGMENTOSA |
| LRBA | Common variable immunodeficiency 8, with autoimmunity |
| LRIT3 | Congenital stationary night blindness, type 1F |
| LRP4 | Cenani-Lenz syndactyly syndrome |
| LRP5 | Exudative vitreoretinopathy 4, Familial exudative vitreoretinopathy, autosomal |
| | dominant |
| LRP6 | 7, Tooth agenesis, selective |
| LRPAP1 | Myopia 23, Rare isolated myopia, autosomal recessive |
| LRPPRC | Congenital lactic acidosis, Saguenay-Lac-Saint-Jean type |
| LRSAM1 | Charcot-Marie-Tooth disease type 2P |
| LRTOMT | Deafness, Rare genetic deafness, autosomal recessive 63 |
| LTBP2 | Congenital glaucoma, Microspherophakia |
| LTBP3 | Dental anomalies and short stature |
| LTBP4 | Cutis laxa with severe pulmonary, and urinary abnormalities, gastrointestinal |
| LYRM7 | Mitochondrial complex III deficiency, nuclear type 8 |
| LZTFL1 | Bardet-Biedl syndrome 17 |
| LZTR1 | Noonan syndrome 2, Schwannomatosis 2 |
| MAB21L1, | AND GENITAL SYNDROME, CEREBELLAR, CRANIOFACIAL, OCULAR |
| NBEA | |
| MAFB | Duane retraction syndrome 2, Duane retraction syndrome 3 with or without |
| | deafness, Duane syndrome type 1, Duane syndrome type 3 |
| MAGED2 | Barrier syndrome, antenatal, transient, type 5 |
| MAGEL2 | Inborn genetic diseases, Schaaf-Yang syndrome |
| MAGT1 | Epstein-Barr virus infection, Immunodeficiency, X-Linked, and neoplasia, with |
| | magnesium defect |
| MAK | Retinal dystrophy |
| MAN2B1 | Deficiency of alpha-mannosidase |
| MANBA | Beta-D-mannosidosis |
| MAP2K2 | Rasopathy |
| MAPRE2 | 2, Skin creases, congenital symmetric circumferential |
| MARVELD2 | Deafness, Rare genetic deafness, autosomal recessive 49, neurosensory |
| MAX | Hereditary cancer-predisposing syndrome |
| MBD5 | Mental retardation, autosomal dominant 1 |
| MC2R | ACTH resistance |
| MC4R | Monogenic diabetes, Obesity, Schizophrenia |
| MCCC1 | 3 Methylcrotonyl-CoA carboxylase 1 deficiency |
| MCCC2 | 3-methylcrotonyl CoA carboxylase 2 deficiency |
| MCM5 | MEIER-GORLIN SYNDROME 8 |
| MCM8 | Premature ovarian failure 10 |
| MCOLN1 | Mucolipidosis type IV |
| MCPH1 | Abnormality of brain morphology, Primary autosomal recessive microcephaly 1 |
| MECP2 | Angelman syndrome, Atypical Rett syndrome, Autism, Delayed gross motor |
| | development, Delayed speech and language development, Developmental |
| | regression, Encephalopathy, Global developmental delay, History of |
| | neurodevelopmental disorder, Inborn genetic diseases, Intellectual disability, Loss |
| | of ability to walk, Mental retardation, Rett syndrome, Severe neonatal-onset |
| | encephalopathy with microcephaly, Smith-Magenis Syndrome-like, Syndromic X- |
| | linked intellectual disability Lubs type, X-linked, X-linked 3, neonatal |
| | severeMental retardation, susceptibility to, syndromic 13, syndromic 13Rett |
| | syndrome |
| MED12 | Cardiovascular phenotype, FG syndrome 1, History of neurodevelopmental |
| | disorder |
| MED13L | Mental retardation and distinctive facial features with or without cardiac defects |
| MED25 | Broad-based gait, Charcot-Marie-Tooth disease, Decreased body weight, Failure to |
| | thrive, Generalized hypotonia, Impaired distal proprioception, Sensory ataxia, |
| | Sensory ataxic neuropathy, Sensory neuropathy, type 2 |
| MEF2C | MEF2C-Related Disorder, Mental retardation, and/or cerebral malformations, |
| | epilepsy, stereotypic movements |
| MEFV | Familial Mediterranean fever |
| MEN1 | Hereditary cancer-predisposing syndrome, Lipoma, Multiple endocrine neoplasia, |
| | somatic, type 1 |
| MERTK | Retinitis pigmentosa 38 |
| MESD | OSTEOGENESIS IMPERFECTA, TYPE XX |
| METTL23 | Inborn genetic diseases, Mental retardation, autosomal recessive 44 |
| MFN2 | Charcot-Marie-Tooth disease, type 2 |
| MFRP, C1QTNF5 | Microphthalmia, Nanophthalmos 2, isolated 5 |
| MFSD8 | Neuronal ceroid lipofuscinosis 7 |
| MIP | Cataract 15, multiple types |
| MIR6886, LDLR | Familial hypercholesterolemia, Familial hypercholesterolemia 1, Homozygous |
| | familial hypercholesterolemia |
| MITF | Coloboma, Rare genetic deafness, Waardenburg syndrome type 2A, albinism, and |
| | deafness, macrocephaly, microphthalmia, osteopetrosis |
| MKRN3 | 2, Precocious puberty, central |
| MKS1 | Joubert syndrome, Joubert syndrome 28, Meckel syndrome type 1, Meckel-Gruber |
| | syndrome |
| MLC1 | Megalencephalic leukoencephalopathy with subcortical cysts 1 |
| MLH1 | Carcinoma of colon, Colon cancer, Hereditary cancer-predisposing syndrome, |
| | Hereditary nonpolyposis colon cancer, Lynch syndrome, Lynch syndrome I, Lynch |
| | syndrome II, Muir-TorrÃ © syndrome, Turcot syndrome |
| MLH3 | Hereditary nonpolyposis colorectal cancer type 7 |
| MLYCD | Deficiency of malonyl-CoA decarboxylase |
| MMAA | Methylmalonic acidemia, Vitamin B12-responsive methylmalonic acidemia type |
| | cblA |
| MMAB | Methylmalonic acidemia, Vitamin B12-responsive methylmalonic acidemia type |
| | cblB |
| MMACHC | DIGENIC, Disorders of Intracellular Cobalamin Metabolism, |
| | METHYLMALONIC ACIDURIA AND HOMOCYSTINURIA, Methylmalonic |
| | acidemia with homocystinuria, Methylmalonic aciduria due to methylmalonyl- |
| | CoA mutase deficiency, cblC TYPE |
| MME | Charcot-Marie-Tooth disease, Congenital membranous nephropathy due to |
| | fetomatemal anti-neutral endopeptidase alloimmunization, axonal, type 2T |
| MMUT | Methylmalonic acidemia, Methylmalonic aciduria due to methylmalonyl-CoA |
| | mutase deficiency |
| MOCS2 | Molybdenum cofactor deficiency, complementation group B |
| MPDZ | 2, Congenital hydrocephalus, Hydrocephalus, congenital, with or without brain or |
| | eye anomalies |
| MPL | Congenital amegakaryocytic thrombocytopenia, essential thrombocytemia |
| MPLKIP | Trichothiodystrophy, nonphotosensitive 1 |
| MPO | Myeloperoxidase deficiency |
| MPV17 | Navajo neurohepatopathy |
| MPZ | Charcot-Marie-Tooth disease |
| MPZL2 | AUTOSOMAL RECESSIVE 111, DEAFNESS |
| MRE11 | Hereditary cancer-predisposing syndrome |
| MSH2 | Carcinoma of colon, Colon cancer, Glioblastoma, Hereditary cancer-predisposing |
| | syndrome, Hereditary nonpolyposis colon cancer, Lynch syndrome, Lynch |
| | syndrome I, Malignant tumor of ascending colon, Malignant tumor of sigmoid |
| | colon, Muir-TorrÃ © syndrome, Ovarian Neoplasms, Turcot syndrome |
| MSH6 | Endometrial carcinoma, Hereditary cancer-predisposing syndrome, Hereditary |
| | nonpolyposis colon cancer, Hereditary nonpolyposis colorectal cancer type 5, |
| | Hereditary nonpolyposis colorectal carcinoma, Lynch syndrome, Lynch syndrome |
| | I, Turcot syndrome |
| MSTO1 | Mitochondrial myopathy-cerebellar ataxia-pigmentary retinopathy syndrome |
| MSX2 | Parietal foramina 1 |
| MTFMT | Abnormal facial shape, Combined oxidative phosphorylation deficiency 15, |
| | Cytochrome C oxidase-negative muscle fibers, Decreased activity of mitochondrial |
| | complex I, Inability to walk by childhood/adolescence, Leigh syndrome, |
| | MITOCHONDRIAL COMPLEX I DEFICIENCY, Mitochondrial oxidative |
| | phosphorylation disorder, NUCLEAR TYPE 27, Poor speech, Short stature |
| MTHFD1 | COMBINED IMMUNODEFICIENCY AND MEGALOBLASTIC ANEMIA |
| | WITH OR WITHOUT HYPERHOMOCYSTEINEMIA |
| MTM1 | Severe X-linked myotubular myopathy |
| MTRR | Disorders of Intracellular Cobalamin Metabolism, Homocystinuria without |
| | methylmalonic aciduria, Homocystinuria-Megaloblastic anemia due to defect in |
| | cobalamin metabolism, cblE complementation type |
| MTTP | Abetalipoproteinaemia |
| MUTYH | Carcinoma of colon, Colon cancer, Familial colorectal cancer, Hereditary cancer- |
| | predisposing syndrome, MUTYH-associated polyposis, MYH-associated |
| | polyposis, Neoplasm of stomach, Pilomatrixoma |
| MVK | Hyperimmunoglobulin D with periodic fever, Mevalonic aciduria, Porokeratosis 3, |
| | disseminated superficial actinic type |
| MYBPC3 | Asymmetric septal hypertrophy, Cardiomyopathy, Cardiovascular phenotype, |
| | Dyspnea, Familial dilated cardiomyopathy, Familial hypertrophic cardiomyopathy |
| | 1, Familial hypertrophic cardiomyopathy 4, Heart block, Hypertrophic |
| | cardiomyopathy, Inborn genetic diseases, Left ventricular hypertrophy, Left |
| | ventricular noncompaction, Left ventricular noncompaction 10, Long QT |
| | syndrome, MYBPC3-Related Disorders, Noncompaction cardiomyopathy, Primary |
| | dilated cardiomyopathy, Primary familial hypertrophic cardiomyopathy, |
| | Tachycardia, Ventricular extrasystoles |
| MYCN | Inborn genetic diseases |
| MYEF2, | Albinism, oculocutaneous, type VI |
| SLC24A5 | |
| MYF5 | Abnormality of the ribs, EXTERNAL, External ophthalmoplegia, |
| | OPHTHALMOPLEGIA, Scoliosis, WITH RIB AND VERTEBRAL |
| | ANOMALIES |
| MYH11, NDE1 | Familial aortopathy |
| MYH2, MYHAS | Myopathy, and ophthalmoplegia, proximal |
| MYH3 | Contractures, Spondylocarpotarsal synostosis syndrome, and variable skeletal |
| | fusions syndrome 1A, pterygia |
| MYH6 | Familial hypertrophic cardiomyopathy 1 |
| MYH7 | Hypertrophic cardiomyopathy, Primary dilated cardiomyopathy |
| MYH7, MHRT | Cardiomyopathy, Cardiovascular phenotype, Hypertrophic cardiomyopathy, |
| | MYH7-Related Disorders |
| MYL2, | Familial hypertrophic cardiomyopathy 10 |
| LOC114827850 | |
| MYLK | Visceral myopathy |
| MYO15A | Congenital sensorineural hearing impairment, Deafness, Nonsyndromic hearing |
| | loss and deafness, Rare genetic deafness, autosomal recessive 3 |
| MYO3A | Deafness, autosomal recessive 30 |
| MYO5B | Congenital microvillous atrophy |
| MYO6 | Deafness, Nonsyndromic hearing loss and deafness, Rare genetic deafness, |
| | autosomal dominant 22 |
| MYO7A | Deafness, MYO7A-Related Disorders, Rare genetic deafness, Retinal dystrophy, |
| | Retinitis pigmentosa, Usher syndrome, Usher syndrome type 1, autosomal |
| | dominant 11, autosomal recessive 2, type 1B |
| MYOCD | CONGENITAL, MEGABLADDER, Prune belly syndrome |
| MYRF | CARDIAC-UROGENITAL SYNDROME |
| NADSYN1 | AND LIMB DEFECTS SYNDROME 3, CARDIAC, Congenital NAD deficiency |
| | disorder, RENAL, VERTEBRAL |
| NAGLU | Charcot-Marie-Tooth disease, MPS-III-B, Mucopolysaccharidosis, Sanfilippo |
| | syndrome, axonal type 2V |
| NALCN | Hypotonia, infantile, with psychomotor retardation and characteristic facies 1 |
| NBAS | Fever-associated acute infantile liver failure syndrome, Infantile liver failure |
| | syndrome 2 |
| NBN | Acute lymphoid leukemia, Aplastic anemia, Breast-ovarian cancer, Familial cancer |
| | of breast, Hereditary breast and ovarian cancer syndrome, Hereditary cancer- |
| | predisposing syndrome, Lissencephaly, Microcephaly, Ovarian Neoplasms, |
| | familial 1, normal intelligence and immunodeficiency |
| NCF1, | Chronic granulomatous disease, Chronic granulomatous disease due to deficiency |
| LOC106029312 | of NCF-1, Granulomatous disease, autosomal recessive, autosomal recessive |
| | cytochrome b-positive, chronic, cytochrome b-positive, type 1, type III |
| NCR1, NLRP7 | 1, Hydatidiform mole, recurrent |
| NCSTN | Familial acne inversa 1 |
| NDE1 | Lissencephaly 4 |
| NDNF | HYPOGONADOTROPIC HYPOGONADISM 25 WITH ANOSMIA |
| NDUFA12 | Leigh syndrome |
| NDUFAF2 | Inborn genetic diseases, Leigh syndrome, MITOCHONDRIAL COMPLEX I |
| | DEFICIENCY, Mitochondrial complex I deficiency, NDUFAF2-Related |
| | Disorders, NUCLEAR TYPE 10, nuclear type 1 |
| NDUFAF3 | Mitochondrial complex I deficiency |
| NDUFB11 | Linear skin defects with multiple congenital anomalies 3 |
| NDUFS4 | Leigh syndrome, Mitochondrial complex I deficiency, nuclear type 1 |
| NDUFS6 | MITOCHONDRIAL COMPLEX I DEFICIENCY, NUCLEAR TYPE 9 |
| NDUFV1 | MITOCHONDRIAL COMPLEX I DEFICIENCY, Mitochondrial complex I |
| | deficiency, NUCLEAR TYPE 4, nuclear type 1 |
| NEB | Inborn genetic diseases, Nemaline myopathy, Nemaline myopathy 2, Non-immune |
| | hydrops fetalis |
| NEB, RIF1 | Nemaline myopathy, Nemaline myopathy 2 |
| NEBL | Hypertrophic cardiomyopathy, Long QT syndrome, Primary dilated |
| | cardiomyopathy, Primary familial hypertrophic cardiomyopathy, Sudden |
| | unexplained death |
| NEFL | Charcot-Marie-Tooth disease type 2E |
| NEK1 | 24, AMYOTROPHIC LATERAL SCLEROSIS, Majewski type, |
| | SUSCEPTIBILITY TO, Short rib-polydactyly syndrome, Short-rib thoracic |
| | dysplasia 3 with or without polydactyly |
| NEUROD1 | Maturity-onset diabetes of the young type 6 |
| NEXN | Dilated cardiomyopathy 1CC, Familial hypertrophic cardiomyopathy 20 |
| NF1 | Axillary freckling, CafÃ ©-au-lait macules with pulmonary stenosis, Focal T2 |
| | hyperintense basal ganglia lesion, Ganglioglioma, Hereditary cancer-predisposing |
| | syndrome, Inborn genetic diseases, Juvenile myelomonocytic leukemia, Multiple |
| | cafe-au-lait spots, Neurofibroma, Neurofibromas, Neurofibromatosis, |
| | Neurofibromatosis-Noonan syndrome, Optic nerve glioma, Pilocytic astrocytoma, |
| | Tibial pseudoarthrosis, familial spinal, type 1 |
| NF1, | Hereditary cancer-predisposing syndrome, Neurofibromatosis, type 1 |
| LOC111811965 | |
| NF2 | Meningioma, Neurofibromatosis, type 2 |
| NFIB | ACQUIRED, Intellectual disability, MACROCEPHALY, Macrocephalus, WITH |
| | IMPAIRED INTELLECTUAL DEVELOPMENT |
| NFIX | Marshall-Smith syndrome |
| NGLY1 | Congenital disorder of deglycosylation, Intellectual disability, Neuromotor delay, |
| | Peripheral neuropathy |
| NHLRC1 | Epilepsy, Lafora disease, progressive myoclonic 2b |
| NHLRC2 | AND CEREBRAL ANGIOMATOSIS, FIBROSIS, NEURODEGENERATION |
| NHS | Nance-Horan syndrome |
| NIPAL4 | Autosomal recessive congenital ichthyosis 6 |
| NIPBL | Cornelia de Lange syndrome 1 |
| NKX2-5 | Abnormality of cardiovascular system morphology, Atrial septal defect 7 with or |
| | without atrioventricular conduction defects |
| NKX3-2 | Spondylo-megaepiphyseal-metaphyseal dysplasia |
| NKX6-2 | AUTOSOMAL RECESSIVE, SPASTIC ATAXIA 8, WITH |
| | HYPOMYELINATING LEUKODYSTROPHY |
| NLGN4X | Autism, Non-syndromic X-linked intellectual disability, X-linked 2, susceptibility |
| | to |
| NLRP7 | 1, Hydatidiform mole, recurrent |
| NOTCH1 | Adams-Oliver syndrome 5, Aortic valve disorder, congenital heart defect |
| NPC1 | Niemann-Pick disease, Niemann-Pick disease type C1, type C |
| NPHP1 | Nephronophthisis, Nephronophthisis 1 |
| NPHP3, NPHP3- | Meckel syndrome type 7 |
| ACAD11 | |
| NPHS1 | Finnish congenital nephrotic syndrome |
| NPHS2 | Idiopathic nephrotic syndrome, Nephrotic syndrome, idiopathic, steroid-resistant |
| NPHS2, | Idiopathic nephrotic syndrome, Nephrotic range proteinuria, Nephrotic syndrome, |
| AXDND1 | idiopathic, steroid-resistant |
| NPRL3, HBA- | Epilepsy, familial focal, with variable foci 3 |
| LCR | |
| NR0B1 | Congenital adrenal hypoplasia, X-linked |
| NR2E3 | Abnormality of color vision, Cone-rod dystrophy, Enhanced s-cone syndrome, |
| | Horizontal nystagmus, NR2E3-Related Disorders, Retinal dystrophy, Retinitis |
| | pigmentosa, Retinitis pigmentosa 37, Visual impairment |
| NR3C2 | Autosomal dominant pseudohypoaldosteronism type 1 |
| NSD1 | Beckwith-Wiedemann syndrome, Inborn genetic diseases, Sotos syndrome 1 |
| NSD2 | 4p partial monosomy syndrome, Wolf-Hirschhom like syndrome |
| NSMCE2 | Seckel syndrome 10 |
| NSMF | Hypogonadotropic hypogonadism 9 with or without anosmia |
| NSUN2 | Mental retardation, autosomal recessive 5 |
| NT5E | Calcification of joints and arteries |
| NTHL1 | Familial adenomatous polyposis 3, Hereditary cancer-predisposing syndrome |
| NTRK1 | Hereditary insensitivity to pain with anhidrosis |
| OAT | Ornithine aminotransferase deficiency |
| OBSL1 | Three M syndrome 2 |
| OCA2 | 1, Skin/hair/eye pigmentation, Tyrosinase-positive oculocutaneous albinism, |
| | variation in |
| OCLN | Pseudo-TORCH syndrome 1 |
| OFD1 | Joubert syndrome, Orofaciodigital syndrome I, Simpson-Golabi-Behmel |
| | syndrome, type 2 |
| OPA1 | Abortive cerebellar ataxia, Dominant hereditary optic atrophy, Inborn genetic |
| | diseases, Mitochondrial diseases, Retinal dystrophy |
| OPHN1 | Mental retardation X-linked with cerebellar hypoplasia and distinctive facial |
| | appearance |
| OPN1LW | Cone monochromatism |
| ORC6 | Meier-Gorlin syndrome 3 |
| OSGIN2, NBN | Hereditary cancer-predisposing syndrome, Microcephaly, normal intelligence and |
| | immunodeficiency |
| OTC | Abnormality of ornithine metabolism, Hyperammonemia, Ornithine |
| | carbamoyltransferase deficiency, Protein avoidance |
| OTOA | Deafness, Rare genetic deafness, autosomal recessive 22 |
| OTOF | Deafness, Rare genetic deafness, autosomal recessive 9 |
| OTOG | Deafness, Intellectual disability, Rare genetic deafness, Seizures, autosomal |
| | recessive 18b |
| OTOGL | Rare genetic deafness |
| OTUD6B | Dysmorphic features, Epilepsy, Intellectual developmental disorder with |
| | dysmorphic facies, Intellectual disability, and distal limb anomalies, seizures |
| OTX2 | Syndromic microphthalmia type 5 |
| P2RY12, | Platelet-type bleeding disorder 8 |
| MED12L | |
| P3H1 | Osteogenesis imperfecta type 8 |
| P3H2 | Myopia, high, with cataract and vitreoretinal degeneration |
| P4HA2 | Myopia 25, autosomal dominant |
| PAFAH1B1 | Inborn genetic diseases, Lissencephaly due to LIS1 mutation |
| PAH | Phenylketonuria |
| PALB2 | Basal cell carcinoma, Breast cancer, Cancer of the pancreas, Familial cancer of |
| | breast, Fanconi anemia, Generalized hypopigmentation, Hereditary breast and |
| | ovarian cancer syndrome, Hereditary cancer, Hereditary cancer-predisposing |
| | syndrome, Neoplasm of the breast, Ovarian Neoplasms, PALB2-Related |
| | Disorders, Pancreatic cancer 3, Pre-B-cell acute lymphoblastic leukemia, |
| | Tracheoesophageal fistula, Tumor susceptibility linked to germline BAP1 |
| | mutations, complementation group N, susceptibility to |
| PANK2 | Pigmentary pallidal degeneration |
| PAPSS2 | Spondyloepimetaphyseal dysplasia, Pakistani type |
| PARN | Dyskeratosis congenita, autosomal recessive 6 |
| PATL2 | OOCYTE MATURATION DEFECT 4 |
| PAX2 | Focal segmental glomerulosclerosis 7, Renal coloboma syndrome |
| PAX3 | Rare genetic deafness, Waardenburg syndrome, Waardenburg syndrome type 1 |
| PAX6 | Aniridia 1, Keratitis, autosomal dominant |
| PAX9 | 3, Tooth agenesis, selective |
| PC | Pyruvate carboxylase deficiency |
| PCCA | Propionic acidemia |
| PCCB | Propionic acidemia |
| PCDH15 | DIGENIC, Deafness, Nonsyndromic Deafness, Rare genetic deafness, Retinal |
| | dystrophy, TYPE ID/F, USHER SYNDROME, Usher syndrome, Usher syndrome |
| | type 1, Usher syndrome type 1D, Usher syndrome type 1F, autosomal recessive |
| | 23, type 1G |
| PCDH19 | Absence seizures, Delayed speech and language development, Early infantile |
| | epileptic encephalopathy 9, Frontal cortical atrophy, Generalized seizures, |
| | Generalized tonic-clonic seizures, Global developmental delay, Hand tremor, Long |
| | palpebral fissure, Prominent fingertip pads, Strabismus, Temporal cortical atrophy |
| PCLO | Pontocerebellar hypoplasia type 3 |
| PCNT | Microcephalic osteodysplastic primordial dwarfism type II |
| PCSK1, | Proprotein convertase 1/3 deficiency |
| LOC101929710 | |
| PCSK9 | Familial hypercholesterolemia, Familial hypercholesterolemia 1, Low density |
| | lipoprotein cholesterol level quantitative trait locus 1 |
| PCYT1A | Spondylometaphyseal dysplasia-cone-rod dystrophy syndrome |
| PDE11A | 2, Pigmented nodular adrenocortical disease, primary |
| PDE6B | Retinal dystrophy, Retinitis pigmentosa, Retinitis pigmentosa 40 |
| PDE6C | Achromatopsia 5 |
| PDE8B | Striatal degeneration, autosomal dominant 1 |
| PDHA1 | Inborn genetic diseases, Pyruvate dehydrogenase E1-alpha deficiency |
| PDX1 | DIABETES MELLITUS, Maturity-onset diabetes of the young type 4, |
| | PERMANENT NEONATAL 1, Pancreatic agenesis 1 |
| PDZD7 | AUTOSOMAL RECESSIVE 57, DEAFNESS, Rare genetic deafness, Usher |
| | syndrome, type 2A |
| PEPD | Prolidase deficiency |
| PEX1 | Deafness enamel hypoplasia nail defects, Peroxisome biogenesis disorder 1A |
| | (Zellweger), Peroxisome biogenesis disorder 1B, Peroxisome biogenesis disorders, |
| | Retinal dystrophy, Zellweger syndrome spectrum |
| PEX1, GATAD1 | Deafness enamel hypoplasia nail defects, Peroxisome biogenesis disorder 1A |
| | (Zellweger), Peroxisome biogenesis disorder 1B, Peroxisome biogenesis disorders, |
| | Zellweger syndrome spectrum |
| PEX10 | Peroxisome biogenesis disorder, Peroxisome biogenesis disorder 6A, Peroxisome |
| | biogenesis disorder 6B, Peroxisome biogenesis disorders, Zellweger syndrome |
| | spectrum, complementation group 7 |
| PEX10, PLCH2 | Peroxisome biogenesis disorder 6B |
| PEX12 | Infantile Refsums disease, Peroxisome biogenesis disorder 3A, Peroxisome |
| | biogenesis disorders, Zellweger syndrome spectrum |
| PEX2 | Peroxisome biogenesis disorder 5B, Peroxisome biogenesis disorder 5a |
| | (zellweger), Peroxisome biogenesis disorders, Zellweger syndrome spectrum |
| PEX26 | Peroxisome biogenesis disorder 7A, Peroxisome biogenesis disorder 7B, |
| | Peroxisome biogenesis disorders, Zellweger syndrome spectrum |
| PEX6 | Heimler syndrome 2, Peroxisome biogenesis disorder 4B, Peroxisome biogenesis |
| | disorder 4a (zellweger), Peroxisome biogenesis disorders, Retinal dystrophy, |
| | Zellweger syndrome spectrum |
| PEX7 | PEX7-Related Disorders, Peroxisome biogenesis disorder 9B, Phytanic acid |
| | storage disease, Rhizomelic chondrodysplasia punctata type 1 |
| PGAM2, DBNL | Glycogen storage disease type X |
| PGAP1 | Mental retardation, autosomal recessive 42 |
| PGAP3 | Hyperphosphatasia with mental retardation syndrome 4 |
| PGM3, DOP1A | Immunodeficiency 23 |
| PHEX | Familial X-linked hypophosphatemic vitamin D refractory rickets |
| PHEX, PTCHD1- | Familial X-linked hypophosphatemic vitamin D refractory rickets |
| AS | |
| PHF3, EYS | Retinal dystrophy, Retinitis pigmentosa 25 |
| PHF6 | Borjeson-Forssman-Lehmann syndrome |
| PHGDH | Phosphoglycerate dehydrogenase deficiency |
| PHIP | Developmental delay, and dysmorphic features, intellectual disability, obesity |
| PHYH | 1, Phytanic acid storage disease, Refsum disease, adult |
| PI4KA | Polymicrogyria, perisylvian, with cerebellar hypoplasia and arthrogryposis |
| PIGA | Paroxysmal nocturnal hemoglobinuria 1 |
| PIGN | Multiple congenital anomalies-hypotonia-seizures syndrome 1 |
| PIGO | Hyperphosphatasia with mental retardation syndrome 2, Hyperphosphatasia- |
| | intellectual disability syndrome |
| PIGT | Multiple congenital anomalies-hypotonia-seizures syndrome 3, PIGT-related |
| | disorder |
| PIK3R1 | SHORT syndrome |
| PINK1 | Parkinson disease 6, autosomal recessive early-onset |
| PIRC66, | Aromatase deficiency |
| MIR4713HG, | |
| CYP19A1 | |
| PITX3 | Anterior segment mesenchymal dysgenesis, Cataract 11 |
| PJVK | Deafness, Rare genetic deafness, autosomal recessive 59 |
| PKD1 | Autosomal recessive polycystic kidney disease, Polycystic kidney disease, adult |
| | type |
| PKD1, | Polycystic kidney disease, adult type |
| LOC105371049 | |
| PKHD1 | Autosomal recessive polycystic kidney disease, Polycystic kidney dysplasia, |
| | Polycystic liver disease |
| PKP1 | Epidermolysis bullosa simplex due to plakophilin deficiency |
| PKP2 | Arrhythmogenic right ventricular cardiomyopathy, Arrhythmogenic right |
| | ventricular dysplasia/cardiomyopathy, Arrhythmogenic ventricular |
| | cardiomyopathy, Cardiac arrhythmia, Cardiomyopathy, Cardiovascular phenotype, |
| | Sudden unexplained death, type 9 |
| PLA2G5 | Fleck retina, familial benign |
| PLA2G6 | Infantile neuroaxonal dystrophy, Iron accumulation in brain, Neurodegeneration |
| | with brain iron accumulation 2b, PLA2G6-associated neurodegeneration |
| PLCB1 | Early infantile epileptic encephalopathy 12 |
| PLCB4 | Auriculocondylar syndrome 2 |
| PLCD1 | Leukonychia totalis |
| PLD1 | Cardiac valvular defect, developmental |
| PLD3, PRX | Charcot-Marie-Tooth disease, SPINOCEREBELLAR ATAXIA 46 |
| PLEC | Epidermolysis bullosa simplex with muscular dystrophy |
| PLN, CEP85L | Cardiac arrest, Cardiomyopathy, Cardiovascular phenotype, Dilated |
| | cardiomyopathy 1P, Familial hypertrophic cardiomyopathy 18, Hypertrophic |
| | cardiomyopathy, Primary dilated cardiomyopathy, Sudden cardiac death |
| PLOD1 | Cardiovascular phenotype, Ehlers-Danlos syndrome, hydroxylysine-deficient |
| PLOD2 | Bruck syndrome 2 |
| PLP1, RAB9B | Hereditary spastic paraplegia 2 |
| PLS3 | Bone mineral density quantitative trait locus 18 |
| PMFBP1 | SPERMATOGENIC FAILURE 31 |
| PMM2 | Congenital disorder of glycosylation, type Ia |
| PMP22 | Charcot-Marie-Tooth disease |
| PMS2 | Acute lymphoid leukemia, Burkitt lymphoma, Colorectal cancer, Glioblastoma, |
| | Hereditary cancer, Hereditary cancer-predisposing syndrome, Hereditary |
| | nonpolyposis colon cancer, Hereditary nonpolyposis colorectal cancer type 4, |
| | Lymphoma, Lynch syndrome, Lynch syndrome I, Pulmonary arterial hypertension, |
| | Pulmonary insufficiency, Respiratory insufficiency, Tumor susceptibility linked to |
| | germline BAP1 mutations, Turcot syndrome, non-polyposis |
| PNKD, CATIP- | Paroxysmal nonkinesigenic dyskinesia 1 |
| AS2 | |
| PNKP | Ataxia-oculomotor apraxia 4, Early infantile epileptic encephalopathy 10, Early |
| | infantile epileptic encephalopathy 12, History of neurodevelopmental disorder |
| PNPLA2 | Neutral lipid storage myopathy |
| PNPLA6 | Hereditary spastic paraplegia 39, Laurence-Moon syndrome, PNPLA6-related |
| | disorders, Trichomegaly-retina pigmentary degeneration-dwarfism syndrome |
| PNPLA8 | Mitochondrial myopathy-lactic acidosis-deafness syndrome |
| PNPO | Pyridoxal phosphate-responsive seizures |
| POC5 | Retinitis pigmentosa, Syndromic retinitis pigmentosa |
| POGLUT1 | Dowling-degos disease 4 |
| POGZ | Global developmental delay, Speech apraxia, White-sutton syndrome, dysmorphy, |
| | intellectual deficiency |
| POLA1 | VAN ESCH-O''''DRISCOLL SYNDROME, Van Esch type, X-linked intellectual |
| | disability |
| POLD1 | Colorectal cancer 10, Hereditary cancer-predisposing syndrome, Mandibular |
| | hypoplasia, and lipodystrophy syndrome, deafness, progeroid features |
| POLE | 12, ADRENAL HYPOPLASIA CONGENITA, AND IMMUNODEFICIENCY, |
| | Colorectal cancer, GENITAL ANOMALIES, Hereditary cancer-predisposing |
| | syndrome, INTRAUTERINE GROWTH RETARDATION, METAPHYSEAL |
| | DYSPLASIA, susceptibility to |
| POLG | Generalized epilepsy, Global developmental delay, Obesity, Progressive sclerosing |
| | poliodystrophy, Seizures |
| POLH | Xeroderma pigmentosum variant type |
| POLR1A | Acrofacial dysostosis, Cincinnati type |
| POLR1C | Treacher Collins syndrome 3 |
| POLR1D | Treacher Collins syndrome 2 |
| POLR2F, SOX10 | Rare genetic deafness, Waardenburg syndrome type 4C |
| POLR3A | Hypomyelinating leukodystrophy 7, Neonatal pseudo-hydrocephalic progeroid |
| | syndrome |
| POLR3B | Cerebellar hypoplasia with endosteal sclerosis, Hypogonadotropic hypogonadism |
| | 7 with or without anosmia, Hypomyelinating leukodystrophy 7, Hypomyelinating |
| | leukodystrophy 8, with or without oligodontia and/or hypogonadotropic |
| | hypogonadism |
| POMK | 12, Muscular dystrophy-dystroglycanopathy (limb-girdle), type c |
| POMT1 | 1, Congenital muscular dystrophy-dystroglycanopathy with mental retardation, |
| | Limb-girdle muscular dystrophy-dystroglycanopathy, Muscular dystrophy- |
| | dystroglycanopathy (congenital with brain and eye anomalies), POMT1-Related |
| | Disorders, Walker-Warburg congenital muscular dystrophy, type A, type B1, type |
| | C1 |
| POMT2 | Congenital muscular dystrophy-dystroglycanopathy with brain and eye anomalies, |
| | Limb-girdle muscular dystrophy-dystroglycanopathy, type A2, type C2 |
| POP1 | Anauxetic dysplasia 2 |
| POR | Antley-Bixler syndrome with genital anomalies and disordered steroidogenesis, |
| | Disordered steroidogenesis due to cytochrome p450 oxidoreductase deficiency |
| PORCN | Focal dermal hypoplasia |
| POT1 | 10, Hereditary cancer-predisposing syndrome, Melanoma, cutaneous malignant, |
| | susceptibility to |
| POU3F4 | Deafness, Rare genetic deafness, X-linked 2 |
| POU4F3 | Rare genetic deafness |
| PPARG | Diabetes Mellitus, Diabetes mellitus, Noninsulin-Dependent, digenic, type II, with |
| | Acanthosis Nigricans and Hypertension |
| PPIB | Osteogenesis imperfecta type 9 |
| PPOX | Variegate porphyria |
| PPP1R12A | GENITOURINARY AND/OR BRAIN MALFORMATION SYNDROME |
| PPT1 | History of neurodevelopmental disorder, Neuronal Ceroid-Lipofuscinosis, |
| | Neuronal ceroid lipofuscinosis, Neuronal ceroid lipofuscinosis 1, Recessive |
| PQBP1 | Delayed speech and language development, Hyperactivity, Inborn genetic diseases, |
| | Intellectual disability, Microcephaly, Renpenning syndrome 1 |
| PRB3 | PRB3M(NULL) |
| PRDM16 | Left ventricular noncompaction 8 |
| PRDM5 | Brittle cornea syndrome 2 |
| PRDX1, | DIGENIC, METHYLMALONIC ACIDURIA AND HOMOCYSTINURIA, cblC |
| MMACHC | TYPE |
| PRF1 | Familial hemophagocytic lymphohistiocytosis, Familial hemophagocytic |
| | lymphohistiocytosis 2 |
| PRKAR1A | Carney complex, type 1 |
| PRKAR1A, | Amelogenesis imperfecta type 1G |
| FAM20A | |
| PRKAR1B, | 18, Ciliary dyskinesia, primary |
| DNAAF5 | |
| PRKCSH | Polycystic liver disease 1 |
| PRKN | Parkinson disease 2 |
| PRMT7 | Short stature, and seizures, brachydactyly, intellectual developmental disability |
| PROK2 | Hypogonadotropic hypogonadism 4 with or without anosmia |
| PROKR2 | Inborn genetic diseases, Kallmann syndrome 3 |
| PROM1 | Cone-rod dystrophy 12, PROM1-Related Disorders, Retinal dystrophy, Retinitis |
| | pigmentosa, Retinitis pigmentosa 41 |
| PROP1 | Pituitary hormone deficiency, combined, combined 2 |
| PRPH2 | Macular dystrophy, Retinal dystrophy, Retinitis pigmentosa 7, Retinitis punctata |
| | albescens, adult-onset, autosomal dominant, vitelliform |
| PRRT2 | 2, Episodic kinesigenic dyskinesia 1, History of neurodevelopmental disorder, |
| | Infantile convulsions and choreoathetosis, Paroxysmal kinesigenic dyskinesia, |
| | Paroxysmal nonkinesigenic dyskinesia 1, Seizures, benign familial infantile |
| PRSS12 | Mental retardation, autosomal recessive 1 |
| PRSS56 | Microphthalmia, isolated 6 |
| PRX | Charcot-Marie-Tooth disease, demyelinating, type 4F |
| PSAP | Combined saposin deficiency |
| PSEN1 | 3, Acne inversa, familial |
| PSENEN | 2, Acne inversa, familial |
| PTCH1 | Gorlin syndrome, Hereditary cancer-predisposing syndrome |
| PTCH2 | Gorlin syndrome, Medulloblastoma |
| PTEN | Cowden syndrome, Cowden syndrome 1, Glioblastoma, Glioma susceptibility 2, |
| | Hemangioma, Hereditary cancer-predisposing syndrome, Inborn genetic diseases, |
| | Macrocephaly/autism syndrome, Malignant tumor of prostate, Meningioma, |
| | Neoplasm of brain, Neoplasm of the breast, Neoplasm of the large intestine, Non- |
| | small cell lung cancer, Ovarian Neoplasms, PTEN hamartoma tumor syndrome, |
| | PTEN-related disorder, Proteus-like syndrome, VACTERL association with |
| | hydrocephalus, familial |
| PTH1R | Chondrodysplasia Blomstrand type |
| PTPN11 | Metachondromatosis |
| PTPRF | 2, Breasts and/or nipples, aplasia or hypoplasia of |
| PTPRO | Nephrotic syndrome, type 6 |
| PTS | BH4-deficient hyperphenylalaninemia A, Hyperphenylalaninemia, a, bh4- |
| | deficient, due to partial pts deficiency |
| PUF60 | Verheij syndrome |
| PURA | Apnea, Generalized hypotonia, Intellectual disability, Limb dystonia, Mental |
| | retardation, PURA Syndrome, PURA-related severe neonatal hypotonia-seizures- |
| | encephalopathy syndrome due to a point mutation, autosomal dominant 31 |
| PUS7 | AND SHORT STATURE, INTELLECTUAL DEVELOPMENTAL DISORDER |
| | WITH ABNORMAL BEHAVIOR, MICROCEPHALY |
| PXDN | Anterior segment dysgenesis 7 |
| PYCR1 | Autosomal recessive cutis laxa type 2B |
| PYGL | Glycogen storage disease, type VI |
| PYGM | Glycogen storage disease, type V |
| RAB23 | Carpenter syndrome, Carpenter syndrome 1 |
| RAB27A | Griscelli syndrome, Griscelli syndrome type 2 |
| RAB33B | Smith-McCort dysplasia 2 |
| RAB3GAP1 | Warburg micro syndrome 1 |
| RABL3 | 5, PANCREATIC CANCER, SUSCEPTIBILITY TO |
| RAD50 | Hereditary cancer-predisposing syndrome, Nijmegen breakage syndrome-like |
| | disorder |
| RAD51C | Breast-ovarian cancer, Fanconi anemia, Hereditary breast and ovarian cancer |
| | syndrome, Hereditary cancer-predisposing syndrome, Ovarian Neoplasms, |
| | RAD51C-Related Disorders, complementation group O, familial 3 |
| RAD51D, | Breast-ovarian cancer, Hereditary breast and ovarian cancer syndrome, Hereditary |
| RAD51L3-RFFL | cancer-predisposing syndrome, Ovarian Neoplasms, familial 4 |
| RAD51L3-RFFL, | Breast-ovarian cancer, Hereditary cancer-predisposing syndrome, familial 4 |
| RAD51D | |
| RAI1 | Smith-Magenis syndrome |
| RAPSN | 11, Myasthenic syndrome, associated with acetylcholine receptor deficiency, |
| | congenital |
| RARS1 | 9, Leukodystrophy, hypomyelinating |
| RASA1 | Capillary malformation-arteriovenous malformation, Capillary malformation- |
| | arteriovenous malformation 1 |
| RB1 | Hereditary cancer-predisposing syndrome, Neoplasm, Osteosarcoma, |
| | Retinoblastoma, Small cell lung cancer, Urinary bladder cancer, trilateral |
| RBBP8 | Microcephaly with mental retardation and digital anomalies |
| RBM20 | Cardiovascular phenotype, Dilated cardiomyopathy 1DD, Primary dilated |
| | cardiomyopathy |
| RBP3 | Retinitis pigmentosa 66 |
| RD3 | Leber congenital amaurosis 12 |
| RDH12 | Retinitis pigmentosa 53 |
| RDH5, | Fundus albipunctatus, Pigmentary retinal dystrophy, autosomal recessive |
| BLOC1S1-RDH5 | |
| RECQL | Hereditary cancer-predisposing syndrome |
| RECQL, | Hereditary cancer-predisposing syndrome |
| PYROXD1 | |
| RECQL4 | B lymphoblastic leukemia lymphoma with t(12; 21)(p13; q22); TEL-AML1 (ETV6- |
| | RUNX1), Baller-Gerold syndrome, High Grade Surface Osteosarcoma, Rapadilino |
| | syndrome, Rothmund-Thomson syndrome, Rothmund-Thomson syndrome type 2 |
| REEP6 | Retinitis pigmentosa 77 |
| RELT | AMELOGENESIS IMPERFECTA, TYPE IIIC |
| REN | Hyperproreninemia, familial |
| RET | Hirschsprung disease 1, Sensorineural hearing loss |
| RFX5 | Bare lymphocyte syndrome, complementation group c, type II |
| RFXANK | Bare lymphocyte syndrome, complementation group B, type II |
| RFXAP | Bare Lymphocyte Syndrome, Bare lymphocyte syndrome 2, Complementation |
| | Group D, Type II |
| RHAG | Rh-null, regulator type |
| RHCE | AMORPH TYPE, RH-NULL |
| RHO | Autosomal dominant retinitis pigmentosa |
| RIF1, NEB | Nemaline myopathy, Nemaline myopathy 2 |
| RIN2 | Macrocephaly, alopecia, and scoliosis, cutis laxa |
| RIPK1 | IMMUNODEFICIENCY 57 WITH AUTOINFLAMMATION |
| RIPK4 | Bartsocas-Papas syndrome |
| RNASEH2A | Aicardi Goutieres syndrome 4 |
| RNASEH2B | Aicardi Goutieres syndrome 2 |
| RNF113A | Trichothiodystrophy 5, nonphotosensitive |
| RNF216 | Gordon Holmes syndrome |
| ROBO3 | Gaze palsy, familial horizontal, with progressive scoliosis 1 |
| RORA, RORA- | INTELLECTUAL DEVELOPMENTAL DISORDER WITH OR WITHOUT |
| AS1 | EPILEPSY OR CEREBELLAR ATAXIA |
| RP1 | Retinal dystrophy, Retinitis pigmentosa, Retinitis pigmentosa 1 |
| RP1L1 | RETINITIS PIGMENTOSA 88 |
| RPE65 | Leber congenital amaurosis 2, RETINITIS PIGMENTOSA 87 WITH |
| | CHOROIDAL INVOLVEMENT, RPE65-Related Disorders, Retinal dystrophy, |
| | Retinitis pigmentosa 20 |
| RPGR | Inborn genetic diseases, Retinal dystrophy, Retinitis pigmentosa, Retinitis |
| | pigmentosa 15, X-linked, and sinorespiratory infections, with deafness |
| RPGRIP1 | Leber congenital amaurosis 6 |
| RPGRIP1L | Joubert syndrome, Joubert syndrome 7 |
| RPL36A- | Fabry disease |
| HNRNPH2, GLA | |
| RPL5, DIPK1A | Diamond-Blackfan anemia, Diamond-Blackfan anemia 1 |
| RPS10, RPS10- | Diamond-Blackfan anemia 9 |
| NUDT3 | |
| RPS27 | Diamond-Blackfan anemia 17 |
| RPS6KA3 | Coffin-Lowry syndrome, Mental retardation, X-linked 19 |
| RSPH1 | Kartagener syndrome, Primary ciliary dyskinesia, Primary ciliary dyskinesia 24 |
| RSPH4A | 11, Ciliary dyskinesia, Kartagener syndrome, Primary ciliary dyskinesia, primary |
| RSPO2 | TETRAAMELIA SYNDROME 2 |
| RTEL1, RTEL1- | 3, 4, 5, Dyskeratosis congenita, Idiopathic fibrosing alveolitis, Pulmonary fibrosis |
| TNFRSF6B | and/or bone marrow failure, autosomal dominant, autosomal recessive, chronic |
| | form, telomere-related |
| RTN2 | Hereditary spastic paraplegia 12 |
| RTTN | Congenital microcephaly, Microcephaly, and polymicrogyria with or without |
| | seizures, short stature |
| RUNX1 | Acute myeloid leukemia, Familial platelet disorder with associated myeloid |
| | malignancy |
| RYR1 | 1, Central core myopathy, Malignant hyperthermia, Minicore myopathy, Multi- |
| | minicore disease and atypical periodic paralysis, Neuromuscular disease, RYR1 - |
| | Related Disorders, susceptibility to |
| SACS | Autosomal recessive spastic ataxia, Charlevoix-Saguenay spastic ataxia, Spastic |
| | paraplegia |
| SAG | Oguchi s disease, Retinitis pigmentosa 47, SAG-Related Disorders |
| SALL1 | Townes syndrome |
| SAMD9L | Ataxia-pancytopenia syndrome |
| SAMHD1 | Aicardi Goutieres syndrome 5 |
| SASH1 | Dyschromatosis universalis hereditaria 1 |
| SATB2 | SATB2-Related Disorder |
| SBDS | Inborn genetic diseases, Shwachman-Diamond syndrome 1 |
| SBF1 | Charcot-Marie-Tooth disease type 4 |
| SCAPER | Attention deficit hyperactivity disorder, INTELLECTUAL DEVELOPMENTAL |
| | DISORDER AND RETINITIS PIGMENTOSA, Intellectual disability, Rod-cone |
| | dystrophy, moderate |
| SCARB2 | Epilepsy, progressive myoclonic 4, with or without renal failure |
| SCARF2 | Van den Ende-Gupta syndrome |
| SCN1A | Autosomal dominant epilepsy, Early infantile epileptic encephalopathy, Familial |
| | hemiplegic migraine type 3, Generalized epilepsy with febrile seizures plus, |
| | History of neurodevelopmental disorder, Severe myoclonic epilepsy in infancy, |
| | type 2, Dravet |
| SCN1A, | Autosomal dominant epilepsy, Early infantile epileptic encephalopathy, Epileptic |
| LOC102724058 | encephalopathy, Generalized epilepsy with febrile seizures plus, Seizures, Severe |
| | myoclonic epilepsy in infancy, type 2 |
| SCN2A | SCN2A-related disorder |
| SCN5A | Brugada syndrome, Brugada syndrome (shorter-than-normal QT interval), Brugada |
| | syndrome 1, Cardiovascular phenotype, Dilated cardiomyopathy 1E, Heart block, |
| | Long QT syndrome 1, nonprogressive |
| SCN5A, | Brugada syndrome, Brugada syndrome (shorter-than-normal QT interval) |
| LOC110121269 | |
| SCN9A, SCN1A- | Generalized epilepsy with febrile seizures plus, Hereditary sensory and autonomic |
| AS1 | neuropathy type IIA, Indifference to pain, autosomal recessive, congenital, type 7 |
| SCNN1A | Autosomal recessive pseudohypoaldosteronism type 1, Idiopathic bronchiectasis |
| SCNN1B | Liddle syndrome 1 |
| SCNN1G | Autosomal recessive pseudohypoaldosteronism type 1, LIDDLE SYNDROME 2 |
| SCO1 | Mitochondrial complex IV deficiency |
| SCP2 | Leukoencephalopathy with dystonia and motor neuropathy |
| SDCCAG8 | Bardet-Biedl syndrome, Bardet-Biedl syndrome 16, Senior-Loken syndrome 7 |
| SDHA | Carney triad, Dilated cardiomyopathy 1GG, Hereditary cancer-predisposing |
| | syndrome, Leigh syndrome, Mitochondrial complex II deficiency, Paragangliomas |
| | 5, Pilocytic astrocytoma |
| SDHAF2 | Hereditary Paraganglioma-Pheochromocytoma Syndromes |
| SDHB | Carney-Stratakis syndrome, Gastrointestinal stromal tumor, Hereditary |
| | Paraganglioma-Pheochromocytoma Syndromes, Hereditary cancer-predisposing |
| | syndrome, Paragangliomas 4, Pheochromocytoma |
| SDHC | Gastrointestinal stromal tumor, Hereditary Paraganglioma-Pheochromocytoma |
| | Syndromes, Hereditary cancer-predisposing syndrome, Paragangliomas 3 |
| SDHD | Carney-Stratakis syndrome, Cowden syndrome 3, Hereditary Paraganglioma- |
| | Pheochromocytoma Syndromes, Hereditary cancer-predisposing syndrome, |
| | Paragangliomas 1, Paragangliomas 1 with sensorineural hearing loss, |
| | Pheochromocytoma |
| SDR9C7 | AUTOSOMAL RECESSIVE 13, CONGENITAL, ICHTHYOSIS |
| SEC23B | Congenital dyserythropoietic anemia |
| SEC24D | Cole-Carpenter syndrome 2 |
| SECISBP2 | Thyroid hormone metabolism, abnormal |
| SELENBP1 | EXTRAORAL HALITOSIS DUE TO METHANETHIOL OXIDASE |
| | DEFICIENCY, Extra oral halitosis |
| SELENON | Eichsfeld type congenital muscular dystrophy |
| SEMA3A | Hypogonadotropic hypogonadism 16 with or without anosmia |
| SEPSECS | Pontocerebellar hypoplasia type 2D |
| SEPTIN12 | Spermatogenic failure 10 |
| SERAC1 | 3-methylglutaconic aciduria with deafness, Mitochondrial oxidative |
| | phosphorylation disorder, and Leigh-like syndrome, encephalopathy |
| SERPINA6 | Corticosteroid-binding globulin deficiency |
| SERPINA7 | Thyroxine-binding globulin quantitative trait locus |
| SERPINB6 | Rare genetic deafness |
| SERPINB7 | Palmoplantar keratoderma, nagashima type |
| SERPINC1 | Antithrombin III deficiency |
| SERPINF1 | Osteogenesis imperfecta, type VI |
| SERPING1 | Hereditary angioedema type 1 |
| SERPINH1 | Osteogenesis imperfecta type 10 |
| SETBP1 | SETBP1-Related Disorder |
| SETD5 | Inborn genetic diseases, Mental retardation, autosomal dominant 23 |
| SF3B4 | Hereditary hearing loss and deafness, Inborn genetic diseases, Nager syndrome |
| SFRP4 | Pyle metaphyseal dysplasia |
| SFTPA1 | Respiratory distress associated with prematurity |
| SFTPB | 1, Surfactant metabolism dysfunction, pulmonary |
| SGCA | Autosomal recessive limb-girdle muscular dystrophy type 2D |
| SGCD | Neuromuscular disease |
| SGCE, CASD1 | Myoclonic dystonia |
| SGCG | Severe autosomal recessive muscular dystrophy of childhood - North African type |
| SGSH | Developmental regression, Diarrhea, Gastrointestinal dysmotility, Global |
| | developmental delay, MPS-III-A, Mucopolysaccharidosis, Nystagmus, Retinal |
| | dystrophy, Sanfilippo syndrome, Severe visual impairment |
| SH2D1A | Lymphoproliferative syndrome 1, X-Linked Lymphoproliferative Syndrome, X- |
| | linked |
| SH3PXD2B | Frank-Ter Haar syndrome |
| SH3TC2 | Charcot-Marie-Tooth disease, Charcot-Marie-Tooth disease type 4, Inborn genetic |
| | diseases, Mononeuropathy of the median nerve, SH3TC2-Related Disorders, mild, |
| | type 4C |
| SHANK3 | 22q13.3 deletion syndrome, Autism spectrum disorder, History of |
| | neurodevelopmental disorder, Inborn genetic diseases, SHANK3-Related Disorder |
| SHOX | Leri-Weill dyschondrosteosis |
| SI | Sucrase-isomaltase deficiency |
| SIX6 | Colobomatous optic disc-macular atrophy-chorioretinopathy syndrome |
| SKIV2L | Trichohepatoenteric syndrome 2 |
| SLC10A7 | AMELOGENESIS IMPERFECTA, AND SKELETAL DYSPLASIA WITH |
| | SCOLIOSIS, SHORT STATURE |
| SLC12A1 | Barrier syndrome, antenatal, type 1 |
| SLC12A3 | Familial hypokalemia-hypomagnesemia |
| SLC12A6 | Agenesis of the corpus callosum with peripheral neuropathy, Charcot-Marie-Tooth |
| | disease |
| SLC17A5 | Salla disease, Sialic acid storage disease, severe infantile type |
| SLC19A1, | Knobloch syndrome 1 |
| COL18A1 | |
| SLC19A2 | Megaloblastic anemia, thiamine-responsive, with diabetes mellitus and |
| | sensorineural deafness |
| SLC19A3 | Biotin-responsive basal ganglia disease |
| SLC22A5 | Renal carnitine transport defect |
| SLC25A20 | Carnitine acylcarnitine translocase deficiency |
| SLC26A2 | 3MC syndrome 2, Achondrogenesis, Atelosteogenesis type II, Diastrophic |
| | dysplasia, Multiple epiphyseal dysplasia type 4, Osteochondrodysplasia, |
| | SLC26A2-Related Disorders, type IB |
| SLC26A3 | Congenital secretory diarrhea, chloride type |
| SLC26A4 | Enlarged vestibular aqueduct, Pendred syndrome, Rare genetic deafness |
| SLC2A10 | Arterial tortuosity syndrome, Cardiovascular phenotype |
| SLC2A2 | Fanconi-Bickel syndrome |
| SLC30A8 | Diabetes mellitus type 2 |
| SLC33A1 | Spastic paraplegia, Spastic paraplegia 42, autosomal dominant |
| SLC34A3 | Autosomal recessive hypophosphatemic bone disease |
| SLC35A2 | SLC35A2-CDG |
| SLC35D1 | Schneckenbecken dysplasia |
| SLC37A4 | Glucose-6-phosphate transport defect, Glycogen storage disease, Inborn genetic |
| | diseases, Phosphate transport defect |
| SLC38A8 | FOVEAL HYPOPLASIA 2 WITH OPTIC NERVE MISROUTING AND |
| | ANTERIOR SEGMENT DYSGENESIS |
| SLC39A4 | Hereditary acrodermatitis enteropathica |
| SLC45A2 | Oculocutaneous albinism type 4 |
| SLC4A1 | Autosomal dominant distal renal tubular acidosis |
| SLC4A11 | 4, Corneal dystrophy, Corneal endothelial dystrophy, Fuchs endothelial |
| SLC52A3 | Brown-Vialetto-Van Laere syndrome 1 |
| SLC6A1 | Myoclonic-atonic epilepsy, SLC6A1-Related Disorder |
| SLC9A3 | Diarrhea 8, congenital, secretory sodium |
| SLC9A3, | Diarrhea 8, congenital, secretory sodium |
| SLC9A3-AS1 | |
| SLC9A6 | Gastrostomy tube feeding in infancy, Global developmental delay, Recurrent |
| | respiratory infections, Scoliosis, Seizures, Sleep disturbance |
| SLCO2A1 | Primary hypertrophic osteoarthropathy, autosomal recessive 2 |
| SLITRK1 | Tourette Syndrome, Trichotillomania |
| SLURP1 | Acroerythrokeratoderma |
| SMAD3 | Familial thoracic aortic aneurysm and aortic dissection |
| SMAD4 | Carcinoma of pancreas, Hereditary cancer-predisposing syndrome, Juvenile |
| | polyposis syndrome, Juvenile polyposis/hereditary hemorrhagic telangiectasia |
| | syndrome, Myhre syndrome |
| SMAD6 | Aortic valve disease 2, Aortic valve disorder, CRANIOSYNOSTOSIS 7, |
| | SUSCEPTIBILITY TO |
| SMARCA4 | Neuroblastoma |
| SMARCAL1 | Schimke immuno-osseous dysplasia |
| SMARCB1 | Teratoid tumor, atypical |
| SMARCE1 | Meningioma, familial |
| SMC1A | 85, Congenital muscular hypertrophy-cerebral syndrome, EARLY INFANTILE, |
| | EPILEPTIC ENCEPHALOPATHY, WITH OR WITHOUT MIDLINE BRAIN |
| | DEFECTS |
| SMN1 | Werdnig-Hoffmann disease |
| SMPD1 | Niemann-Pick disease, Sphingomyelin/cholesterol lipidosis, type A, type B |
| SNAP29 | 2, CEDNIK syndrome, Leukodystrophy, hypomyelinating |
| SNRPB | Cerebro-costo-mandibular syndrome |
| SOHLH1 | Nonsyndromic hypergonadotropic hypogonadism, OVARIAN DYSGENESIS 5 |
| SON | Inborn genetic diseases, ZTTK syndrome |
| SOS1 | Gingival fibromatosis 1 |
| SOX2, SOX2-OT | Anophthalmia/microphthalmia-esophageal atresia syndrome |
| SOX9 | Campomelic dysplasia with autosomal sex reversal, Camptomelic dysplasia |
| SOX9, | Campomelic dysplasia with autosomal sex reversal |
| LOC108021846 | |
| SP110, SP140 | Hepatic veno-occlusive disease-immunodeficiency syndrome |
| SP7 | Osteogenesis imperfecta type 12 |
| SPART | Troyer syndrome |
| SPAST | Spastic paraplegia 4, autosomal dominant |
| SPEF2 | Primary ciliary dyskinesia, SPERMATOGENIC FAILURE 43 |
| SPEG | 5, Myopathy, centronuclear |
| SPEG, ASIC4- | 5, Myopathy, centronuclear |
| AS1 | |
| SPG11 | Amyotrophic lateral sclerosis type 5, Hereditary spastic paraplegia, Spastic |
| | paraplegia 11, autosomal recessive |
| SPG7 | Hereditary spastic paraplegia, Hereditary spastic paraplegia 7, Mitochondrial |
| | diseases |
| SPINK2 | Spermatogenic failure 29 |
| SPINK5 | Netherton syndrome |
| SPNS2 | AUTOSOMAL RECESSIVE 115, DEAFNESS, Inborn genetic diseases |
| SPRTN | Ruijs-Aalfs syndrome |
| SPTA1 | Elliptocytosis 2, Hereditary pyropoikilocytosis |
| SPTB | Hereditary spherocytosis, Spherocytosis type 2 |
| SQSTM1 | Amyotrophic lateral sclerosis and/or frontotemporal dementia 1, Paget disease of |
| | bone 2, SQSTM1-related disorder, early-onset |
| SRCAP | Floating-Harbor syndrome |
| SRPK2, KMT2E | See cases |
| SRY | 46, XY sex reversal, type 1 |
| ST14 | Ichthyosis, autosomal recessive 11, congenital |
| STAG1 | AUTOSOMAL DOMINANT 47, MENTAL RETARDATION |
| STAG3 | Abnormality of the ovary, Female infertility, Premature ovarian failure 8, |
| | Premature ovarian insufficiency |
| STAT1 | Mycobacterial and viral infections, autosomal recessive, susceptibility to |
| STIM1 | 1, Combined immunodeficiency due to STIM1 deficiency, Myopathy, Stormorken |
| | syndrome, tubular aggregate |
| STK11 | Hereditary cancer-predisposing syndrome, Peutz-Jeghers syndrome |
| STRA6 | Microphthalmia syndromic 9 |
| STRC | Deafness, Rare genetic deafness, autosomal recessive 16 |
| STXBP1 | Early infantile epileptic encephalopathy, Early infantile epileptic encephalopathy |
| | 4, Epileptic encephalopathy |
| STXBP2 | 5, Hemophagocytic lymphohistiocytosis, familial |
| SUCLG1 | Mitochondrial DNA depletion syndrome 9 (encephalomyopathic with |
| | methylmalonic aciduria) |
| SUFU | Gorlin syndrome, Medulloblastoma, Medulloblastoma with extensive nodularity, |
| | desmoplastic |
| SULT2B1 | AUTOSOMAL RECESSIVE 14, Autosomal recessive congenital ichthyosis 2, |
| | CONGENITAL, ICHTHYOSIS |
| SUMF1 | Multiple sulfatase deficiency |
| SUN5 | Spermatogenic failure 16 |
| SURF1 | Abnormal pyramidal signs, Cerebellar ataxia, Charcot-Marie-Tooth disease, |
| | Dysarthria, Inborn genetic diseases, Leigh syndrome, Leigh syndrome due to COX |
| | IV deficiency, Leigh syndrome due to mitochondrial complex IV deficiency, |
| | Mitochondrial complex IV deficiency, Muscle weakness, type 4k |
| SUZ12 | IMAGAWA-MATSUMOTO SYNDROME |
| SYCP2 | Cryptozoospermia, Early spermatogenesis maturation arrest, Oligosynaptic |
| | infertility |
| SYCP3 | Spermatogenic failure 4 |
| SYNE1 | ARTHROGRYPOSIS MULTIPLEX CONGENITA, Cerebellar ataxia, Emery- |
| | Dreifuss muscular dystrophy 4, MYOGENIC TYPE, Spinocerebellar ataxia, |
| | autosomal dominant, autosomal recessive 8 |
| SYNE4 | Rare genetic deafness |
| SYNGAP1 | Inborn genetic diseases, Mental retardation, autosomal dominant 5 |
| SZT2 | Early infantile epileptic encephalopathy 18 |
| TAC3 | Hypogonadotropic hypogonadism 10 with or without anosmia |
| TACO1 | Mitochondrial complex IV deficiency |
| TALDO1 | Deficiency of transaldolase |
| TANGO2 | AND NEURODEGENERATION, Acute rhabdomyolysis, CARDIAC |
| | ARRHYTHMIAS, Cardiac arrhythmia, Episodic flaccid weakness, Intellectual |
| | functioning disability, METABOLIC CRISES, RECURRENT, Seizures, WITH |
| | RHABDOMYOLYSIS |
| TAP1 | Bare lymphocyte syndrome type 1 |
| TAP2 | Bare lymphocyte syndrome type 1, PEPTIDE TRANSPORTER PSF2 |
| | POLYMORPHISM |
| TAZ | 3-Methylglutaconic aciduria type 2 |
| TBC1D20 | Warburg micro syndrome 4 |
| TBC1D24 | 1, Caused by mutation in the TBC1 domain family, DOORS syndrome, Deafness, |
| | Epileptic encephalopathy, Inborn genetic diseases, autosomal dominant 65, early |
| | infantile, member 24 |
| TBCK | Hypotonia, Inborn genetic diseases, Syndromic Infantile Encephalopathy, infantile, |
| | with psychomotor retardation and characteristic facies 3 |
| TBR1 | Autism 5, Autistic behavior, Intellectual disability, Moderate global developmental |
| | delay, Neurodevelopmental disorder, Severe global developmental delay |
| TBX19 | Adrenocorticotropic hormone deficiency |
| TBX22 | Cleft palate with ankyloglossia |
| TBX3 | Ulnar-mammary syndrome |
| TBX4 | Coxopodopatellar syndrome |
| TBX5 | Congenital heart disease (variable), Holt-Oram syndrome |
| TBXAS1 | Ghosal hematodiaphyseal dysplasia, Thromboxane synthetase deficiency |
| TCAP | Autosomal recessive limb-girdle muscular dystrophy type 2G, Dilated |
| | cardiomyopathy 1N, Primary familial hypertrophic cardiomyopathy |
| TCF12 | Craniosynostosis 3 |
| TCF20 | Neurodevelopmental abnormality |
| TCF4 | Intellectual disability, Pitt-Hopkins syndrome |
| TCN2 | Inborn genetic diseases, Transcobalamin II deficiency |
| TCOF1 | Treacher Collins syndrome 1 |
| TCTEX1D2 | Short-rib thoracic dysplasia 17 with or without polydactyly |
| TCTEX1D2, | Short-rib thoracic dysplasia 17 with or without polydactyly |
| TM4SF19- | |
| TCTEX1D2 | |
| TCTN2 | Joubert syndrome, Meckel syndrome type 8 |
| TCTN3 | Orofacial-digital syndrome IV |
| TDO2 | Hypertryptophanemia, familial |
| TDRD7 | Cataract, autosomal recessive congenital 4 |
| TDRD9 | SPERMATOGENIC FAILURE 30 |
| TECPR2 | Spastic paraplegia 49, autosomal recessive |
| TECTA | Deafness, Nonsyndromic hearing loss and deafness, Rare genetic deafness, |
| | autosomal dominant 12, autosomal recessive 21, neurosensory autosomal recessive |
| | 21 |
| TENM3 | MICROPHTHALMIA, SYNDROMIC 15 |
| TENT5A | Osteogenesis imperfecta, type 18 |
| TEX14 | SPERMATOGENIC FAILURE 23 |
| TEX15 | SPERMATOGENIC FAILURE 25 |
| TFAP2B | Patent ductus arteriosus 2 |
| TFR2 | Hemochromatosis type 3 |
| TG | Iodotyrosyl coupling defect |
| TGFB2 | Cardiovascular phenotype, Holt-Oram syndrome, Loeys-Dietz syndrome 4 |
| TGFB3 | Cardiovascular phenotype, Loeys-Dietz syndrome 5 |
| TGFBR1 | Familial thoracic aortic aneurysm and aortic dissection |
| TGFBR2 | Familial thoracic aortic aneurysm and aortic dissection, Hereditary nonpolyposis |
| | colorectal cancer type 6, Loeys-Dietz syndrome, Loeys-Dietz syndrome 2, |
| | Malignant tumor of esophagus |
| TGM1 | Autosomal recessive congenital ichthyosis 1, Ichthyosis (disease) |
| TGM5 | Peeling skin syndrome 2 |
| TH | Segawa syndrome, autosomal recessive |
| THRB | Thyroid hormone resistance, autosomal dominant, generalized |
| TICAM1 | 4, Herpes simplex encephalitis, susceptibility to |
| TIMM8A | Deafness dystonia syndrome |
| TIMMDC1 | Leigh syndrome |
| TJP2 | Progressive familial intrahepatic cholestasis 4 |
| TK2 | Mitochondrial DNA depletion syndrome 2 |
| TLR5 | 1, Legionellosis, Melioidosis, Systemic lupus erythematosus, resistance to |
| TM4SF20 | Specific language impairment 5 |
| TMC1 | Deafness, Dominant, Nonsyndromic Hearing Loss, Rare genetic deafness, |
| | autosomal recessive 7 |
| TMCO1 | Craniofacial dysmorphism, and mental retardation syndrome, skeletal anomalies |
| TMCO6, | Cystic Leukoencephalopathy |
| NDUFA2 | |
| TMEM127 | Hereditary Paraganglioma-Pheochromocytoma Syndromes, Hereditary cancer- |
| | predisposing syndrome, Pheochromocytoma |
| TMEM216 | Joubert syndrome, Joubert syndrome 2, Meckel syndrome, TMEM216-Related |
| | Disorders, type 2 |
| TMEM237 | Joubert syndrome |
| TMEM260 | Structural heart defects and renal anomalies syndrome |
| TMEM67 | Cerebellar vermis hypoplasia, Generalized hypotonia, Iris coloboma, Joubert |
| | syndrome, Joubert syndrome 6, Meckel syndrome, Meckel-Gruber syndrome, |
| | Nystagmus, TMEM67-Related Disorders, type 3 |
| TMEM70 | Mitochondrial proton-transporting ATP synthase complex deficiency, Nuclearly- |
| | encoded mitochondrial complex V (ATP synthase) deficiency 2 |
| TMEM94 | Intellectual developmental disorder with cardiac defects and dysmorphic facies |
| TMEM99, KRT10 | Bullous ichthyosiform erythroderma |
| TMPRSS3 | Deafness, Inborn genetic diseases, Rare genetic deafness, autosomal recessive 8 |
| TNFRSF10B | Squamous cell carcinoma of the head and neck |
| TNFRSF11B | Hyperphosphatasemia with bone disease |
| TNFRSF13B | Absent epiphyses, Chronic lung disease, Cleft palate, Clubfoot, Coat hanger sign |
| | of ribs, Common Variable Immune Deficiency, Common variable |
| | immunodeficiency 2, Dominant, Hemivertebrae, Immunoglobulin A deficiency 2, |
| | Interstitial pulmonary abnormality, Micrognathia, Patent ductus arteriosus, |
| | Preaxial foot polydactyly, Pseudoarthrosis, Respiratory failure, Short femur, |
| | Skeletal dysplasia, Vertebral hypoplasia, Vertebral segmentation defect |
| TNFRSF1A | 5, Familial Periodic Fever, Multiple sclerosis, susceptibility to |
| TNFSF11 | Autosomal recessive osteopetrosis 2 |
| TNNI3 | Cardiovascular phenotype |
| TNNI3K, FPGT- | Cardiac conduction disease with or without dilated cardiomyopathy |
| TNNI3K | |
| TNNT2 | Cardiomyopathy, Cardiovascular phenotype, Familial hypertrophic |
| | cardiomyopathy 2, Familial restrictive cardiomyopathy 3, Hypertrophic |
| | cardiomyopathy, Left ventricular noncompaction 6, Primary familial hypertrophic |
| | cardiomyopathy |
| TNPO3 | Limb-girdle muscular dystrophy, type 1F |
| TNXB | 1, Ehlers-Danlos syndrome, Ehlers-Danlos syndrome due to tenascin-X deficiency, |
| | classic-like |
| TONSL | Sponastrime dysplasia |
| TONSL, TONSL- | Sponastrime dysplasia |
| AS1 | |
| TOP3A | AND INCREASED SISTER CHROMATID EXCHANGE 2, GROWTH |
| | RESTRICTION, MICROCEPHALY |
| TOPORS | Retinal dystrophy, Retinitis pigmentosa |
| TP53 | Head and Neck Neoplasms, Hereditary cancer-predisposing syndrome, Li- |
| | Fraumeni syndrome, Li-Fraumeni syndrome 1, Li-Fraumeni-like syndrome, |
| | Multiple myeloma, Neoplasm of the large intestine, Ovarian Neoplasms |
| TP63 | Ectrodactyly, Orofacial cleft 8, and cleft lip/palate syndrome 3, ectodermal |
| | dysplasia |
| TPI1 | Triosephosphate isomerase deficiency |
| TPM2 | ARTHROGRYPOSIS, DISTAL, TYPE 2B4 |
| TPO | Deficiency of iodide peroxidase |
| TPP1 | Ceroid lipofuscinosis neuronal 2, Childhood-onset autosomal recessive slowly |
| | progressive spinocerebellar ataxia, Inborn genetic diseases, Neuronal ceroid |
| | lipofuscinosis |
| TPRN | Deafness, autosomal recessive 79 |
| TRAPPC11 | Limb-girdle muscular dystrophy, type 2S |
| TRAPPC2 | Spondyloepiphyseal dysplasia tarda |
| TRDN | 5, Catecholaminergic polymorphic ventricular tachycardia, Ventricular |
| | tachycardia, catecholaminergic polymorphic, with or without muscle weakness |
| TREX1, ATRIP, | Aicardi Goutieres syndrome 1, Chilblain Lupus, Retinal vasculopathy with |
| ATRIP-TREX1 | cerebral leukoencephalopathy and systemic manifestations, TREX1-Related |
| | Disorders |
| TRIM14, NANS | Genevieve type, Spondyloepimetaphyseal dysplasia |
| TRIM32, ASTN2 | Limb-girdle muscular dystrophy |
| TRIOBP | Nonsyndromic hearing loss and deafness |
| TRIP11 | Achondrogenesis, Goldblatt hypertension, Osteochondrodysplasia, type IA |
| TRMU | Acute infantile liver failure due to synthesis defect of mtDNA-encoded proteins |
| TRNT1 | Retinitis pigmentosa and erythrocytic microcytosis, Sideroblastic anemia with B- |
| | cell immunodeficiency, and developmental delay, periodic fevers |
| TRPM4 | Cardiomyopathy, Progressive familial heart block type IB, TRPM4-Related |
| | Disorders |
| TRPS1 | Trichorhinophalangeal dysplasia type I |
| TRPV4 | Charcot-Marie-Tooth disease axonal type 2C |
| TRPV6 | HYPERPARATHYROIDISM, TRANSIENT NEONATAL |
| TSC1 | Cortical dysplasia, Cortical tubers, Focal cortical dysplasia type II, Hereditary |
| | cancer-predisposing syndrome, Lymphangiomyomatosis, Multiple renal cysts, |
| | Renal cortical cysts, Renal insufficiency, Seizures, Tuberous sclerosis 1, Tuberous |
| | sclerosis syndrome, Urinary bladder cancer |
| TSC2 | Focal cortical dysplasia type II, Lymphangiomyomatosis, Tuberous sclerosis 2, |
| | Tuberous sclerosis syndrome |
| TSFM | Combined oxidative phosphorylation deficiency 3, Primary dilated |
| | cardiomyopathy |
| TSHB | Secondary hypothyroidism |
| TSHR | 1, Hypothyroidism, congenital, nongoitrous |
| TSHZ1 | Aural atresia, congenital |
| TSPAN1, | Congenital muscular alpha-dystroglycanopathy with brain and eye anomalies, |
| POMGNT1 | Congenital muscular dystrophy-dystroglycanopathy with mental retardation, Limb- |
| | girdle muscular dystrophy-dystroglycanopathy, Muscle eye brain disease, |
| | POMGNT1-Related Disorders, Retinitis pigmentosa 76, type B3, type C3 |
| TSPAN12 | Exudative vitreoretinopathy 5 |
| TSPAN7 | Mental retardation 58, X-linked |
| TSPEAR | ECTODERMAL DYSPLASIA 14, HAIR/TOOTH TYPE WITH |
| | HYPOHIDROSIS |
| TSPEAR-AS1, | Deafness, ECTODERMAL DYSPLASIA 14, HAIR/TOOTH TYPE WITH |
| TSPEAR | HYPOHIDROSIS, autosomal recessive 98 |
| TTC19 | Mitochondrial complex III deficiency, nuclear type 2 |
| TTC21A | SPERMATOGENIC FAILURE 37 |
| TTC21B, | SHORT-RIB THORACIC DYSPLASIA 4 WITH POLYDACTYLY |
| TTC21B-AS1 | |
| TTC29 | SPERMATOGENIC FAILURE 42 |
| TTC37 | Trichohepatoenteric syndrome, Trichohepatoenteric syndrome 1 |
| TTC7A | Multiple gastrointestinal atresias |
| TTLL5 | Cone-rod dystrophy 19 |
| TTN | Cardiomyopathy, Cardiovascular phenotype, Dilated cardiomyopathy 1G, Limb- |
| | girdle muscular dystrophy, Myotubular myopathy, Primary dilated |
| | cardiomyopathy, Tibial muscular dystrophy, type 2J |
| TTN-AS1, TTN | Cardiovascular phenotype, Dilated cardiomyopathy 1G, Limb-girdle muscular |
| | dystrophy, Primary dilated cardiomyopathy, TTN-Related Disorders, type 2J |
| TTN, | Primary dilated cardiomyopathy |
| LOC101927055 | |
| TTN, TTN-AS1 | 9, Broad-based gait, Cardiomyopathy, Cardiovascular phenotype, Congenital |
| | muscular dystrophy, Decreased patellar reflex, Delayed gross motor development, |
| | Dilated cardiomyopathy 1G, Dilated cardiomyopathy 1S, Distal muscle weakness, |
| | Familial dilated cardiomyopathy, Familial hypertrophic cardiomyopathy 9, Gowers |
| | sign, Heart murmur, Limb-girdle muscular dystrophy, Muscular dystrophy, |
| | Myopathy, Primary dilated cardiomyopathy, Proximal lower limb amyotrophy, |
| | Scoliosis, Severe muscular hypotonia, TTN-Related disorder, Tibial muscular |
| | dystrophy, Waddling gait, early-onset, myofibrillar, type 2J, with early respiratory |
| | failure, with fatal cardiomyopathy |
| TTPA | Ataxia, Familial isolated deficiency of vitamin E, Friedreich-like, with isolated |
| | vitamin E deficiency |
| TUB, RIC3 | Retinal dystrophy and obesity |
| TUBA3D, | KERATOCONUS 9 |
| MZT2A | |
| TUBB8 | Oocyte maturation defect 2 |
| TULP1 | Leber congenital amaurosis, Retinitis pigmentosa |
| TWIST1 | Craniosynostosis 1, Robinow-Sorauf syndrome, Saethre-Chotzen syndrome |
| TXNL4A | Burn-McKeown syndrome |
| TYK2 | Tyrosine kinase 2 deficiency |
| TYR | 3, Albinism, Inborn genetic diseases, Myopia (disease), Nonsyndromic |
| | Oculocutaneous Albinism, Nystagmus, Oculocutaneous albinism, Oculocutaneous |
| | albinism type 1B, Skin/hair/eye pigmentation, Tyrosinase-negative oculocutaneous |
| | albinism, ocular, variation in, with sensorineural deafness |
| TYRP1, | Oculocutaneous albinism type 3 |
| LURAP1L-AS1 | |
| UBAP1 | AUTOSOMAL DOMINANT, SPASTIC PARAPLEGIA 80 |
| UBE3A, SNHG14 | Angelman syndrome, History of neurodevelopmental disorder, Inborn genetic |
| | diseases |
| UBE3B | Kaufman oculocerebrofacial syndrome |
| UBR1 | Johanson-Blizzard syndrome |
| UCP3 | Obesity, and type II diabetes, severe |
| UGT1A, | Crigler-Najjar syndrome, Crigler-Najjar syndrome type 1, type II |
| UGT1A10, | |
| UGT1A8, | |
| UGT1A7, | |
| UGT1A6, | |
| UGT1A5, | |
| UGT1A9, | |
| UGT1A4, | |
| UGT1A1, | |
| UGT1A3 | |
| UNC13D | Familial hemophagocytic lymphohistiocytosis 3 |
| UNC80 | Hypotonia, Hypotonia-speech impairment-severe cognitive delay syndrome, |
| | infantile, with psychomotor retardation and characteristic facies 2 |
| UNG | Hyper-IgM syndrome type 5 |
| UPF3B | Mental retardation, X-linked, syndromic 14 |
| USH1C | Deafness, Rare genetic deafness, Retinal dystrophy, Retinitis pigmentosa, Usher |
| | syndrome, Usher syndrome type 1, autosomal recessive 18, type 1C |
| USH2A | Abnormality of the upper limb, Abnormality of upper limb bone, Abnormality of |
| | upper limb joint, Anxiety, Brisk reflexes, Chronic pain, Cognitive impairment, |
| | Cone-rod dystrophy, Congenital sensorineural hearing impairment, Congenital |
| | stationary night blindness, Dislocated radial head, Distal arthrogryposis, |
| | Dysautonomia, Hearing impairment, High palate, Inborn genetic diseases, Macular |
| | dystrophy, Multiple joint contractures, Rare genetic deafness, Retinal dystrophy, |
| | Retinitis pigmentosa, Retinitis pigmentosa 39, Short stature, USH2A-Related |
| | Disorders, Usher syndrome, Usher syndrome type 2, type 2A |
| USH2A, USH2A- | Rare genetic deafness, Retinal dystrophy, Retinitis pigmentosa 39, USH2A- |
| AS1 | Related Disorders, Usher syndrome, type 2A |
| USH2A, USH2A- | Rare genetic deafness, Retinitis pigmentosa 39, Usher syndrome, type 2A |
| AS2 | |
| USP18 | Pseudo-TORCH syndrome 2 |
| USP27X | Mental retardation, X-linked 105 |
| USP9X | Mental retardation, USP9X related disorders, X-linked 99, female-restricted, |
| | syndromic |
| VCL | Dilated cardiomyopathy 1W, Familial hypertrophic cardiomyopathy 15, Primary |
| | dilated cardiomyopathy |
| VHL | 2, Erythrocytosis, Hereditary cancer-predisposing syndrome, Von Hippel-Lindau |
| | syndrome, familial |
| VHL, | 1, 2, Erythrocytosis, Hereditary cancer-predisposing syndrome, Renal cell |
| LOC107303340 | carcinoma, Von Hippel-Lindau syndrome, familial, papillary |
| VIM, VIM-AS1 | Cataract 30, Congenital cataract |
| VIPAS39 | Arthrogryposis, and cholestasis 2, renal dysfunction |
| VPS13A | Choreoacanthocytosis |
| VPS13B | Abnormality of the eye, Cohen syndrome, Inborn genetic diseases, Intellectual |
| | disability, Microcephaly, Neutropenia, Progressive visual loss, Recurrent aphthous |
| | stomatitis, Retinal dystrophy, Short foot, Short stature, Small hand |
| VPS33B | Arthrogryposis, Inborn genetic diseases, and cholestasis 1, renal dysfunction |
| VRK2, FANCL | Fanconi anemia, complementation group A, complementation group L |
| VWF | von Willebrand disorder |
| WAC | Desanto-shinawi syndrome |
| WAS | Wiskott-Aldrich syndrome, X-linked severe congenital neutropenia, X-linked |
| | thrombocytopenia with normal platelets |
| WDR35 | Cranioectodermal dysplasia, Cranioectodermal dysplasia 2, Jeune thoracic |
| | dystrophy, SHORT-RIB THORACIC DYSPLASIA 7 WITHOUT |
| | POLYDACTYLY, Short Rib Polydactyly Syndrome, Short rib polydactyly |
| | syndrome 5, Short-rib thoracic dysplasia 7/20 with polydactyly, WDR35-Related |
| | Disorders, digenic |
| WDR45 | Neurodegeneration with brain iron accumulation, Neurodegeneration with brain |
| | iron accumulation 5 |
| WDR72 | Amelogenesis imperfecta |
| WDR73 | Galloway-Mowat syndrome 1 |
| WEE2-AS1, | OOCYTE MATURATION DEFECT 5 |
| WEE2 | |
| WFS1 | Autosomal dominant nonsyndromic deafness 6, Diabetes mellitus AND insipidus |
| | with optic atrophy AND deafness, WFS1-Related Spectrum Disorders, Wolfram- |
| | like syndrome, autosomal dominant |
| WHRN | Deafness, Rare genetic deafness, Usher syndrome, autosomal recessive 31, type |
| | 2D |
| WRN | Medulloblastoma, Werner syndrome |
| WT1 | Drash syndrome, Frasier syndrome, Wilms tumor, Wilms tumor 1, and mental |
| | retardation syndrome, aniridia, genitourinary anomalies |
| WT1, | Drash syndrome, Frasier syndrome, Pre-B-cell acute lymphoblastic leukemia, |
| LOC107982234 | Wilms tumor, Wilms tumor 1, and mental retardation syndrome, aniridia, |
| | genitourinary anomalies |
| XDH | Deficiency of xanthine oxidase |
| XIAP | Lymphoproliferative syndrome 2, X-linked |
| XK | McLeod neuroacanthocytosis syndrome |
| XPA | Xeroderma pigmentosum, Xeroderma pigmentosum group A |
| XPC | Xeroderma pigmentosum, group C |
| XRCC2 | Fanconi anemia, Hereditary Cancer Syndrome, Hereditary breast and ovarian |
| | cancer syndrome, Hereditary cancer-predisposing syndrome, Ovarian Neoplasms, |
| | complementation group U |
| XRCC4 | Short stature, and endocrine dysfunction, microcephaly |
| XYLT1 | Desbuquois dysplasia 2 |
| XYLT1, | Desbuquois dysplasia 2 |
| LOC102723692 | |
| XYLT2 | Inborn genetic diseases, Spondyloocular syndrome, autosomal recessive |
| YY1AP1 | Grange syndrome |
| ZBTB18 | Mental retardation, autosomal dominant 22 |
| ZDBF2 | Nasopalpebral lipoma-coloboma syndrome |
| ZEB2 | Mowat-Wilson syndrome |
| ZFYVE26 | Hereditary spastic paraplegia 15, Spastic paraplegia |
| ZFYVE26, | Abnormality of the eye, Leber congenital amaurosis 13, RDH12-Related |
| RDH12 | Disorders, Retinal dystrophy, Retinitis pigmentosa |
| ZMPSTE24 | Lethal tight skin contracture syndrome, Mandibuloacral dysplasia with type B |
| | lipodystrophy, ZMPSTE24-Related Disorders |
| ZNF408 | Retinitis pigmentosa 72 |
| ZNF462 | Craniosynostosis, Mental retardation, WEISS-KRUSZKA SYNDROME, |
| | autosomal dominant |
| ZNF711 | ZNF711-Related X-linked Mental Retardation |
| ZP1 | Oocyte maturation defect 1 |
| ZP2 | OOCYTE MATURATION DEFECT 6 |

In an embodiment, the disease or disorder associated with a PTC is a lysosomal storage disease (e.g., Fabry disease, Gaucher disease, or Niemann-Pick disease). In some embodiments, the disease or disorder associated with a PTC is Fabry disease. In an embodiment, upon administration of a TREM (e.g., a TREM described herein) to a cell or subject, the level of a GLA protein in the cell or subject is modulated, e.g., increased, by about 0.1%, 0.5%, 1%, 2%, 3%, 4% 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., compared with a reference value (e.g., level of a GLA protein in a healthy, non-Fabry disease fibroblast).

In some embodiments, the disease or disorder associated with a PTC is a blood clotting disorder, e.g., Hemophilia B. In an embodiment, upon administration of a TREM (e.g., a TREM described herein) to a cell or subject, the level of a Factor IX (FIX) protein in the cell or subject is modulated, e.g., increased, by about 0.1%, 0.5%, 1%, 2%, 3%, 4% 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., compared with a reference value (e.g., level of a FIX protein in a healthy, non-disease cell).

In some embodiments, the disease or disorder associated with a PTC is an autosomal recessive disorder, such as neuronal ceroid lipofuscinosis type 2 (CNL2). In an embodiment, upon administration of a TREM (e.g., a TREM described herein) to a cell or subject, the level of a tripeptidyl peptidase 1 (TPP1) protein in the cell or subject is modulated, e.g., increased, by about 0.1%, 0.5%, 1%, 2%, 3%, 4% 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., compared with a reference value (e.g., level of a TPP1 protein in a healthy, non-disease cell).

In some embodiments, the disease or disorder associated with a PTC is a disease or disorder associated with hearing loss, such as Usher syndrome (e.g., Usher syndrome type 1F). In an embodiment, upon administration of a TREM (e.g., a TREM described herein) to a cell or subject, the level of a protocadherin 15 precursor (PCDH15) protein in the cell or subject is modulated, e.g., increased, by about 0.1%, 0.5%, 1%, 2%, 3%, 4% 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., compared with a reference value (e.g., level of a PCDH15 protein in a healthy, non-disease cell).

In an embodiment, the disease or disorder associated with a PTC is a proliferative disease, such as a benign neoplasm or a cancer. In an embodiment, the proliferative disease is associated with a benign neoplasm. For example, a benign neoplasm may include adenoma, fibroma, hemangioma, tuberous sclerosis, and lipoma. All types of benign neoplasms disclosed herein or known in the art are contemplated as being within the scope of the disclosure.

In an embodiment, the proliferative disease is a cancer. As used herein, the term “cancer” refers to a malignant neoplasm (Stedman's Medical Dictionary, 25th ed.; Hensyl ed.; Williams & Wilkins: Philadelphia, 1990). All types of cancers disclosed herein or known in the art are contemplated as being within the scope of the disclosure. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer), e.g., adenoid cystic carcinoma (ACC)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypercosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; parancoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva). In some embodiments, the cancer is a solid tumor, such as a sarcoma or a carcinoma (e.g., lung cancer, brain cancer, breast cancer, bladder cancer, prostate cancer, colon cancer, rectal cancer).

In another aspect, the present disclosure features methods of treating a disease or disorder in a cell or subject by administration of a TREM (e.g., a TREM described herein) to the cell or subject. Exemplary diseases or disorders include hemophilias, aminoacidopathies, metal storage disorders, peroxisome biogenesis disorder, progressive rare lung disease, diseases related to lipid metabolism, diseases related to galactose metabolism, systemic organic acidemias, urea cycle disorders, cholestastis disorders, bilirubin metabolism disorders, lysososomal storage disorders, glycogen storage diseases, and oxalate metabolism disorders. In an embodiment, the disease or disorder is a hemophilia, e.g., hemophilia A or hemophilia B. In an embodiment, the disease or disorder is an aminoacidopathy, e.g., tyrosinemia type 1, tyrosinemia type 2, tyrosinemia type 3, maple syrup urine disease, alkaptonuria, or phenylketonuria. In an embodiment, the disease or disorder is a systemic organic acidemia, e.g., methylmalonic acidemia (MMUT), methylmalonic acidemia (non-MMUT), propionic acidemia type A, propionic acidemia type B, or isovaleric acidemia. In an embodiment, the disease or disorder is a urea cycle disorder, e.g. argininosuccinate lyase deficiency, argininosuccinate lyase deficiency-D, citrullinemia type 1, citrullinemia type 2, carbamoyl phosphate synthetase-D, ornithine transcarbamylase, arginemia, or hyperornithinemia-hyperammonemia-homocitrullinuria (HHH) syndrome. In an embodiment, the disease or disorder is lysosomal storage disorder, e.g., mucopolysaccharidosis 1, mucopolysaccharidosis 2. Fabry disease, lysosomal acid lipas deficiency, Pompe disease, Gaucher disease, Niemann Pick A, or Niemann Pick B. In an embodiment, the disease or disorder is a bilirubin metabolism disorder, e.g., Crigler-Najjar syndrome. In an embodiment, the disease or disorder is a cholestastis disorder, e.g., progressive familial intrahepatic cholestasis (PFIC) type 1, PFIC type 2, or PFIC type 3. In an embodiment, the disease or disorder is a disease related to lipid metabolism, e.g., sitosterolemia (ABCG5) or sitosterolemia (ABCG8). In an embodiment, the disease or disorder is a glycogen storage disease, e.g., glycogen storage disease 1a, glycogen storage disease 1b, or glycogen storage disease 3a. In an embodiment, the disease or disorder is a metal storage disorder, e.g., Wilson disease or hereditary hemochromatosis. In an embodiment, the disease or disorder is a progressive rare lung disease, e.g., alpha-1 antitrypsin deficiency. In an embodiment, the disease or disorder is a peroxisome biogenesis disorder, e.g., PBD RCDP1. In an embodiment, the disease or disorder is an oxalate metabolism disorder, e.g. primary hyperoxaluria type 1, primary hyperoxaluria type 2, or primary hyperoxaluria type 3. In an embodiment, the disease or disorder is a congenital disorder related to Notch signaling, e.g., Alagille syndrome. In an embodiment, the disease or disorder is an amyloidosis, e.g., familial amyloid polyneuropathy.

In one aspect, the present disclosure features a method of treating a disease or disorder in a subject, the method comprising administering to the subject a TREM comprising the nucleotide sequence of any one of the TREMS listed in FIG. 2. In an embodiment, the disease or disorder is selected from a hemophilia, aminoacidopathy, metal storage disorder, peroxisome biogenesis disorder, progressive rare lung disease, disease related to lipid metabolism, disease related to galactose metabolism, systemic organic acidemia, urea cycle disorder, cholestastis disorder, bilirubin metabolism disorder, lysososomal storage disorder, glycogen storage disease, and oxalate metabolism disorder. In an embodiment, the TREM comprises the sequence of any one of SEQ ID NO: 622, 623, 624, 4249, 4386, 4834, 5630, 6707, 6749, 6947, or 8051, or a fragment or variant thereof. [WILL EXPAND HERE A BIT]

### Method of Making TREMs, TREM Core Fragments, and TREM Fragments

In vitro methods for synthesizing oligonucleotides are known in the art and can be used to make a TREM, a TREM core fragment or a TREM fragment disclosed herein. For example, a TREM, TREM core fragment or TREM fragment can be synthesized using solid state synthesis or liquid phase synthesis.

In an embodiment, a TREM, a TREM core fragment or a TREM fragment made according to an in vitro synthesis method disclosed herein has a different modification profile compared to a TREM expressed and isolated from a cell, or compared to a naturally occurring tRNA.

An exemplary method for making a modified TREM is provided in Example 1. The method provided in Example 1 can also be used to make a synthetic TREM core fragment or synthetic TREM fragment. Additional synthetic methods are disclosed in Hartsel SA et al., (2005) Oligonucleotide Synthesis, 033-050, the entire contents of which are hereby incorporated by reference.

### TREM Composition

In an embodiment, a TREM composition, e.g., a TREM pharmaceutical composition, comprises a pharmaceutically acceptable excipient. Exemplary excipients include those provided in the FDA Inactive Ingredient Database (https://www.accessdata.fda.gov/scripts/cder/iig/index. Cfm).

In an embodiment, a TREM composition, e.g., a TREM pharmaceutical composition, comprises at least 1, 2, 3, 4, 5, 6, 7, 8, 9, 10, 15, 20, 30, 40, 50, 60, 70, 80, 90, 100 or 150 grams of TREM, TREM core fragment or TREM fragment. In an embodiment, a TREM composition, e.g., a TREM pharmaceutical composition, comprises at least 1, 2, 3, 4, 5, 6, 7, 8, 9, 10, 15, 20, 30, 40, 50 or 100 milligrams of TREM, TREM core fragment or TREM fragment.

In an embodiment, a TREM composition, e.g., a TREM pharmaceutical composition, is at least 10, 20, 30, 40, 50, 60, 70, 80, 90, 95 or 99% dry weight TREMs, TREM core fragments or TREM fragments.

In an embodiment, a TREM composition comprises at least 1×10⁶ TREM molecules, at least 1×10⁷ TREM molecules, at least 1×10⁸ TREM molecules or at least 1×10⁹ TREM molecules.

In an embodiment, a TREM composition comprises at least 1×10⁶ TREM core fragment molecules, at least 1×10⁷ TREM core fragment molecules, at least 1×10⁸ TREM core fragment molecules or at least 1×10⁹ TREM core fragment molecules.

In an embodiment, a TREM composition comprises at least 1×10⁶ TREM fragment molecules, at least 1×10⁷ TREM fragment molecules, at least 1×10⁸ TREM fragment molecules or at least 1×10⁹ TREM fragment molecules.

In an embodiment, a TREM composition produced by any of the methods of making disclosed herein can be charged with an amino acid using an in vitro charging reaction as known in the art.

In an embodiment, a TREM composition comprise one or more species of TREMs, TREM core fragments, or TREM fragments. In an embodiment, a TREM composition comprises a single species of TREM. TREM core fragment, or TREM fragment. In an embodiment, a TREM composition comprises a first TREM, TREM core fragment, or TREM fragment species and a second TREM, TREM core fragment, or TREM fragment species. In an embodiment, the TREM composition comprises X TREM, TREM core fragment, or TREM fragment species, wherein X=2, 3, 4, 5, 6, 7, 8, 9, or 10.

In an embodiment, the TREM, TREM core fragment, or TREM fragment has at least 70, 75, 80, 85, 90, or 95, or has 100%, identity with a sequence encoded by a nucleic acid in Table 1.

In an embodiment, the TREM comprises a consensus sequence provided herein.

A TREM composition can be formulated as a liquid composition, as a lyophilized composition or as a frozen composition.

In some embodiments, a TREM composition can be formulated to be suitable for pharmaceutical use, e.g., a pharmaceutical TREM composition. In an embodiment, a pharmaceutical TREM composition is substantially free of materials and/or reagents used to separate and/or purify a TREM, TREM core fragment, or TREM fragment.

In some embodiments, a TREM composition can be formulated with water for injection. In some embodiments, a TREM composition formulated with water for injection is suitable for pharmaceutical use, e.g., comprises a pharmaceutical TREM composition.

### TREM Characterization

A TREM, TREM core fragment, or TREM fragment, or a TREM composition, e.g., a pharmaceutical TREM composition, produced by any of the methods disclosed herein can be assessed for a characteristic associated with the TREM, TREM core fragment, or TREM fragment or the TREM composition, such as purity, sterility, concentration, structure, or functional activity of the TREM, TREM core fragment, or TREM fragment. Any of the above-mentioned characteristics can be evaluated by providing a value for the characteristic, e.g., by evaluating or testing the TREM, TREM core fragment, or TREM fragment, or the TREM composition, or an intermediate in the production of the TREM composition. The value can also be compared with a standard or a reference value. Responsive to the evaluation, the TREM composition can be classified, e.g., as ready for release, meets production standard for human trials, complies with ISO standards, complies with cGMP standards, or complies with other pharmaceutical standards. Responsive to the evaluation, the TREM composition can be subjected to further processing, e.g., it can be divided into aliquots, e.g., into single or multi-dosage amounts, disposed in a container, e.g., an end-use vial, packaged, shipped, or put into commerce. In embodiments, in response to the evaluation, one or more of the characteristics can be modulated, processed or re-processed to optimize the TREM composition. For example, the TREM composition can be modulated, processed or re-processed to (i) increase the purity of the TREM composition; (ii) decrease the amount of fragments in the composition; (iii) decrease the amount of endotoxins in the composition; (iv) increase the in vitro translation activity of the composition; (v) increase the TREM concentration of the composition; or (vi) inactivate or remove any viral contaminants present in the composition, e.g., by reducing the pH of the composition or by filtration.

In an embodiment, the TREM, TREM core fragment, or TREM fragment (e.g., TREM composition or an intermediate in the production of the TREM composition) has a purity of at least 30%, 40%, 50%, 60%, 70%, 80%, 85%, 90%, 91%, 92%, 93%, 94%, 95%, 96%, 97%, 98%, or 99%, i.e., by mass.

In an embodiment, the TREM (e.g., TREM composition or an intermediate in the production of the TREM composition) has less than 0.1%, 0.5%, 1%, 2%, 3%, 4%, 5%, 6%, 7%, 8%, 9%, 10%, 15%, 20%, 25% TREM fragments relative to full length TREMs.

In an embodiment, the TREM, TREM core fragment, or TREM fragment (e.g., TREM composition or an intermediate in the production of the TREM composition) has low levels or absence of endotoxins, e.g., a negative result as measured by the Limulus amebocyte lysate (LAL) test.

In an embodiment, the TREM. TREM core fragment, or TREM fragment (e.g., TREM composition or an intermediate in the production of the TREM composition) has in-vitro translation activity, e.g., as measured by an assay described in Examples 12-13.

In an embodiment, the TREM, TREM core fragment, or TREM fragment (e.g., TREM composition or an intermediate in the production of the TREM composition) has a TREM concentration of at least 0.1 ng/mL, 0.5 ng/ml, 1 ng/ml, 5 ng/ml, 10 ng/ml, 50 ng/ml, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL. 100 ug/mL, 200 ug/mL, 300 ug/mL, 500 ug/mL, 1000 ug/mL, 5000 ug/mL, 10,000 ug/mL, or 100,000 ug/mL.

In an embodiment, the TREM, TREM core fragment, or TREM fragment (e.g., TREM composition or an intermediate in the production of the TREM composition) is sterile, e.g., the composition or preparation supports the growth of fewer than 100 viable microorganisms as tested under aseptic conditions, the composition or preparation meets the standard of USP <71>, and/or the composition or preparation meets the standard of USP <85>.

In an embodiment, the TREM, TREM core fragment, or TREM fragment (e.g., TREM composition or an intermediate in the production of the TREM composition) has an undetectable level of viral contaminants, e.g., no viral contaminants. In an embodiment, any viral contaminant, e.g., residual virus, present in the composition is inactivated or removed. In an embodiment, any viral contaminant, e.g., residual virus, is inactivated, e.g., by reducing the pH of the composition. In an embodiment, any viral contaminant, e.g., residual virus, is removed, e.g., by filtration or other methods known in the field.

### TREM Administration

Any TREM composition or pharmaceutical composition described herein can be administered to a cell, tissue or subject, e.g., by direct administration to a cell, tissue and/or an organ in vitro, ex-vivo or in vivo. In-vivo administration may be via, e.g., by local, systemic and/or parenteral routes, for example intravenous, subcutaneous, intraperitoneal, intrathecal, intramuscular, ocular, nasal, urogenital, intradermal, dermal, enteral, intravitreal, intracerebral, intrathecal, or epidural.

### Vectors and Carriers

In some embodiments the TREM, TREM core fragment, or TREM fragment or TREM composition described herein, is delivered to cells, e.g. mammalian cells or human cells, using a vector. The vector may be, e.g., a plasmid or a virus. In some embodiments, delivery is in vivo, in vitro, ex vivo, or in situ. In some embodiments, the virus is an adeno associated virus (AAV), a lentivirus, or an adenovirus. In some embodiments, the system or components of the system are delivered to cells with a viral-like particle or a virosome. In some embodiments, the delivery uses more than one virus, viral-like particle or virosome.

### Carriers

A TREM, a TREM composition or a pharmaceutical TREM composition described herein may comprise, may be formulated with, or may be delivered in, a carrier.

### Viral Vectors

The carrier may be a viral vector (e.g., a viral vector comprising a sequence encoding a TREM, a TREM core fragment or a TREM fragment). The viral vector may be administered to a cell or to a subject (e.g., a human subject or animal model) to deliver a TREM, a TREM core fragment or a TREM fragment, a TREM composition or a pharmaceutical TREM composition.

A viral vector may be systemically or locally administered (e.g., injected). Viral genomes provide a rich source of vectors that can be used for the efficient delivery of exogenous genes into a mammalian cell. Viral genomes are known in the art as useful vectors for delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus, replication deficient herpes virus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology (Third Edition) Lippincott-Raven, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the teachings of which are incorporated herein by reference. In some embodiments the system or components of the system are delivered to cells with a viral-like particle or a virosome.

### Cell and Vesicle-Based Carriers

A TREM, a TREM core fragment or a TREM fragment, a TREM composition or a pharmaceutical TREM composition described herein can be administered to a cell in a vesicle or other membrane-based carrier.

In embodiments, a TREM, a TREM core fragment or a TREM fragment, or TREM composition, or pharmaceutical TREM composition described herein is administered in or via a cell, vesicle or other membrane-based carrier. In one embodiment, the TREM, TREM core fragment, TREM fragment, or TREM composition or pharmaceutical TREM composition can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi: 10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi: 10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Lipid nanoparticles are another example of a carrier that provides a biocompatible and biodegradable delivery system for the TREM, TREM core fragment, TREM fragment, or TREM composition or pharmaceutical TREM composition described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi: 10.3390/nano7060122.

Exemplary lipid nanoparticles are disclosed in International Application PCT/US2014/053907, the entire contents of which are hereby incorporated by reference. For example, an LNP described in paragraphs [403-406] or [410-413] of PCT/US2014/053907 can be used as a carrier for the TREM, TREM core fragment, TREM fragment, or TREM composition or pharmaceutical TREM composition described herein.

Additional exemplary lipid nanoparticles are disclosed in U.S. Pat. No. 10,562,849 the entire contents of which are hereby incorporated by reference. For example, an LNP of formula (I) as described in columns 1-3 of U.S. Pat. No. 10,562,849 can be used as a carrier for the TREM, TREM core fragment, TREM fragment, or TREM composition or pharmaceutical TREM composition described herein.

Lipids that can be used in nanoparticle formations (e.g., lipid nanoparticles) include, for example those described in Table 4 of WO2019217941, which is incorporated by reference, e.g., a lipid-containing nanoparticle can comprise one or more of the lipids in Table 4 of WO2019217941. Lipid nanoparticles can include additional elements, such as polymers, such as the polymers described in Table 5 of WO2019217941, incorporated by reference.

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di (tetradecanoyloxy) propyl-1-O-(w-methoxy (polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), and combinations of the foregoing.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al (2020), incorporated herein by reference.

In some embodiments, the lipid particle comprises an ionizable lipid, a non-cationic lipid, a conjugated lipid that inhibits aggregation of particles, and a sterol. The amounts of these components can be varied independently and to achieve desired properties. For example, in some embodiments, the lipid nanoparticle comprises an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid can be varied as desired. For example, the total lipid to nucleic acid (mass or weight) ratio can be from about 10:1 to about 30:1.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Some non-limiting example of lipid compounds that may be used (e.g., in combination with other lipid components) to form lipid nanoparticles for the delivery of compositions described herein, e.g., nucleic acid (e.g., RNA) described herein includes,

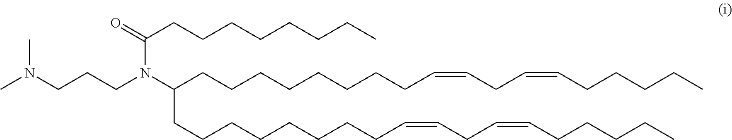 [SMILES: CCCCC/C=C\C/C=C\CCCCCCCCC(CCCCCCCC/C=C\C/C=C\CCCCC)N(CCCN(C)C)C(=O)CCCCCCCC]

In some embodiments an LNP comprising Formula (i) is used to deliver a TREM composition described herein to the liver and/or hepatocyte cells.

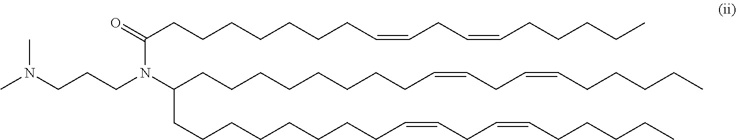 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC]

In some embodiments an LNP comprising Formula (ii) is used to deliver a TREM composition described herein to the liver and/or hepatocyte cells.

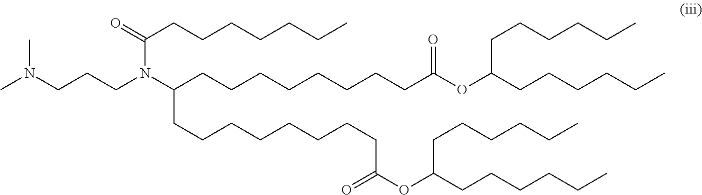 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

In some embodiments an LNP comprising Formula (iii) is used to deliver a TREM composition described herein to the liver and/or hepatocyte cells.

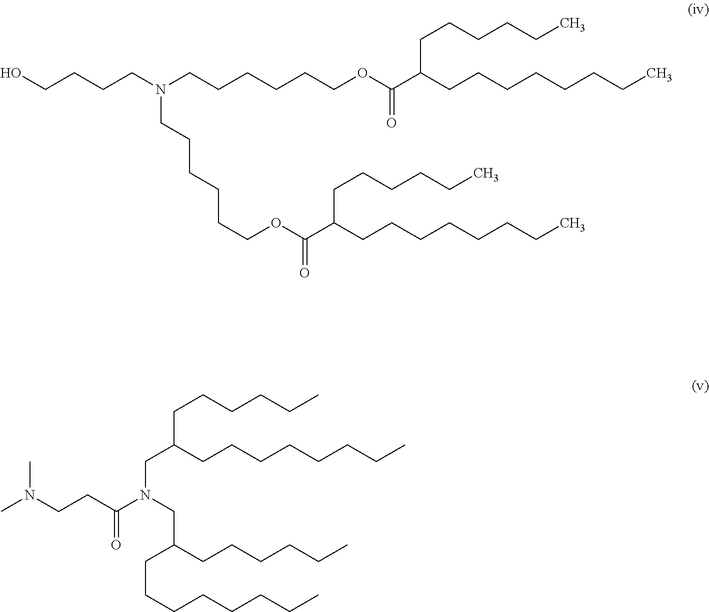

In some embodiments an LNP comprising Formula (v) is used to deliver a TREM composition described herein to the liver and/or hepatocyte cells.

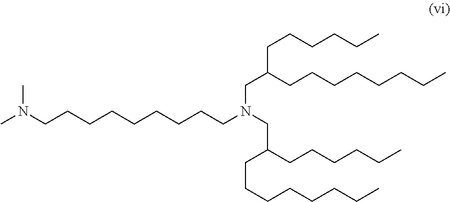 [SMILES: CCCCCCCCC(CCCCCC)CN(CCCCCCCCCN(C)C)CC(CCCCCC)CCCCCCCC]

In some embodiments an LNP comprising Formula (vi) is used to deliver a TREM composition described herein to the liver and/or hepatocyte cells.

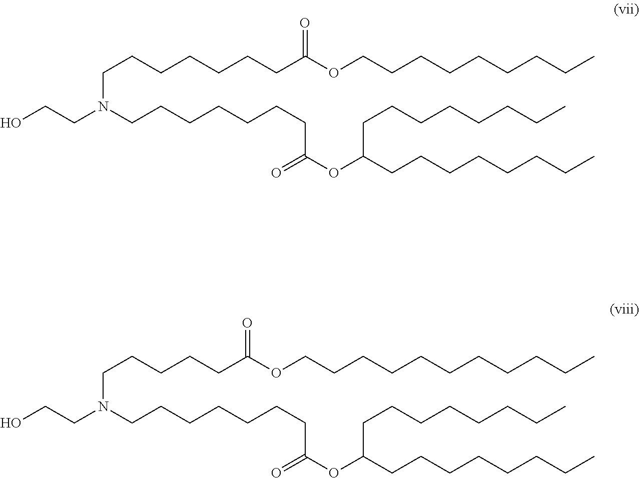 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC]

In some embodiments an LNP comprising Formula (viii) is used to deliver a TREM composition described herein to the liver and/or hepatocyte cells.

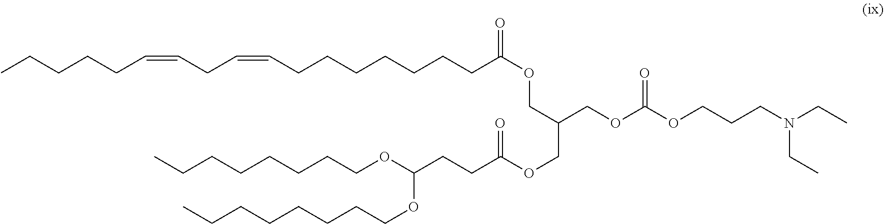 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments an LNP comprising Formula (ix) is used to deliver a TREM composition described herein to the liver and/or hepatocyte cells.

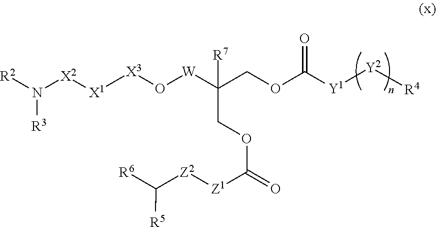

wherein X¹ is O, NR¹, or a direct bond, X² is C2-5 alkylene, X³ is C(═O) or a direct bond, R¹ is H or Me, R³ is C1-3 alkyl, R² is C1-3 alkyl, or R² taken together with the nitrogen atom to which it is attached and 1-3 carbon atoms of X² form a 4-, 5-, or 6-membered ring, or X¹ is NR¹, R¹ and R² taken together with the nitrogen atoms to which they are attached form a 5- or 6-membered ring, or R² taken together with R³ and the nitrogen atom to which they are attached form a 5-, 6-, or 7-membered ring, Y¹ is C2-12 alkylene, Y² is selected from

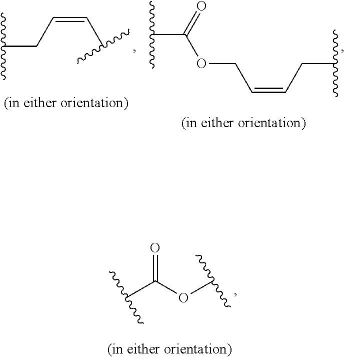 [SMILES: */C=C\C* ; *C/C=C\COC(*)=O ; *OC(*)=O]

n is 0 to 3, R⁴ is C1-15 alkyl, Z¹ is C1-6 alkylene or a direct bond, Z² is

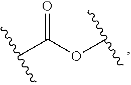 [SMILES: *OC(*)=O]

(in either orientation) or absent, provided that if Z′ is a direct bond, Z² is absent; R⁵ is C5-9 alkyl or C6-10 alkoxy, R⁶ is C5-9 alkyl or C6-10 alkoxy, W is methylene or a direct bond, and R⁷ is H or Me, or a salt thereof, provided that if R³ and R² are C2 alkyls, X¹ is O, X² is linear C3 alkylene, X³ is C(=0), Y¹ is linear Ce alkylene, (Y²) n-R⁴ is:

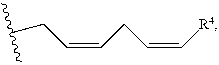

R⁴ is linear C5 alkyl, Z¹ is C2 alkylene, Z² is absent, W is methylene, and R⁷ is H, then R⁵ and R⁶ are not Cx alkoxy.

In some embodiments an LNP comprising Formula (xii) is used to deliver a TREM composition described herein to the liver and/or hepatocyte cells.

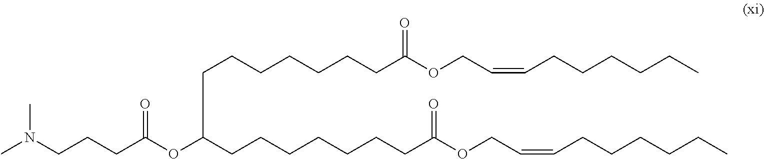 [SMILES: CCCCCC/C=C\COC(=O)CCCCCCCC(CCCCCCCC(=O)OC/C=C\CCCCCC)OC(=O)CCCN(C)C]

In some embodiments an LNP comprising Formula (xi) is used to deliver a TREM composition described herein to the liver and/or hepatocyte cells.

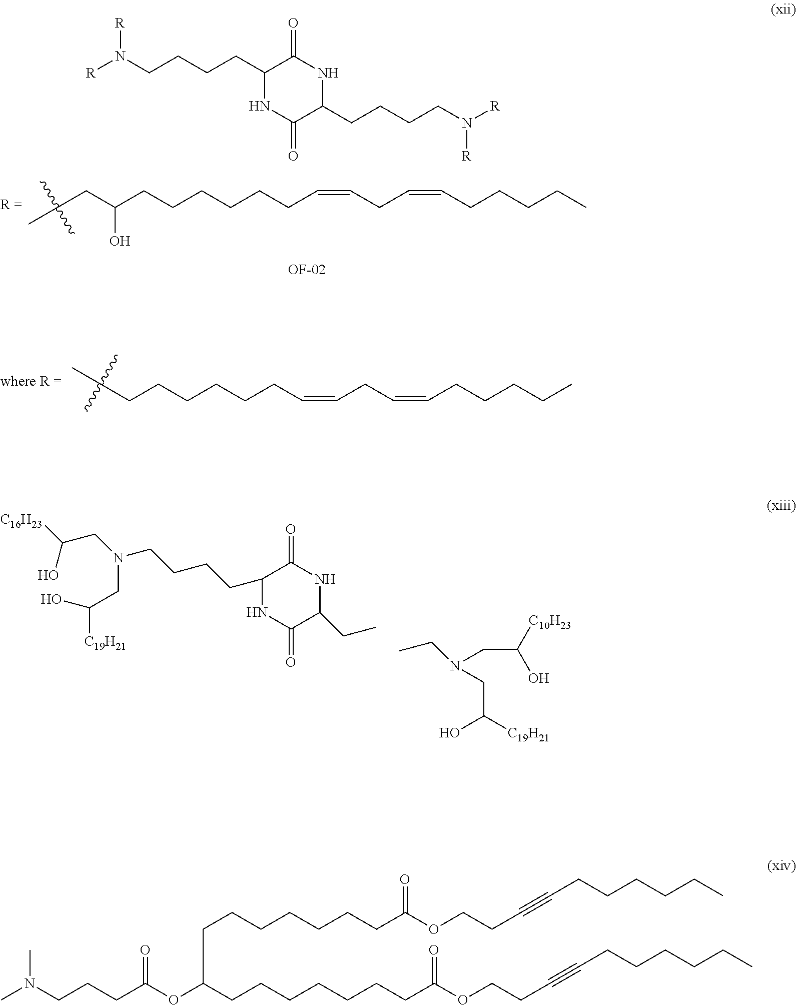

In some embodiments an LNP comprises a compound of Formula (xiii) and a compound of Formula (xiv).

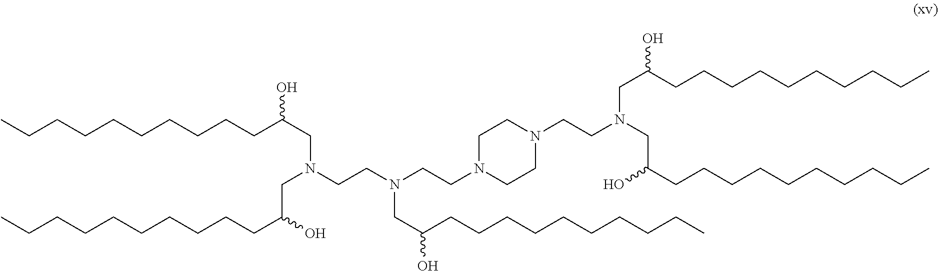 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments, an LNP comprising Formula (xv) is used to deliver a TREM composition described herein to the liver and/or hepatocyte cells.

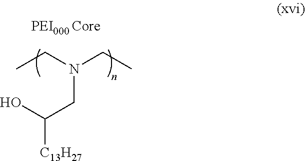

In some embodiments an LNP comprising a formulation of Formula (xvi) is used to deliver a TREM composition described herein to the lung endothelial cells.

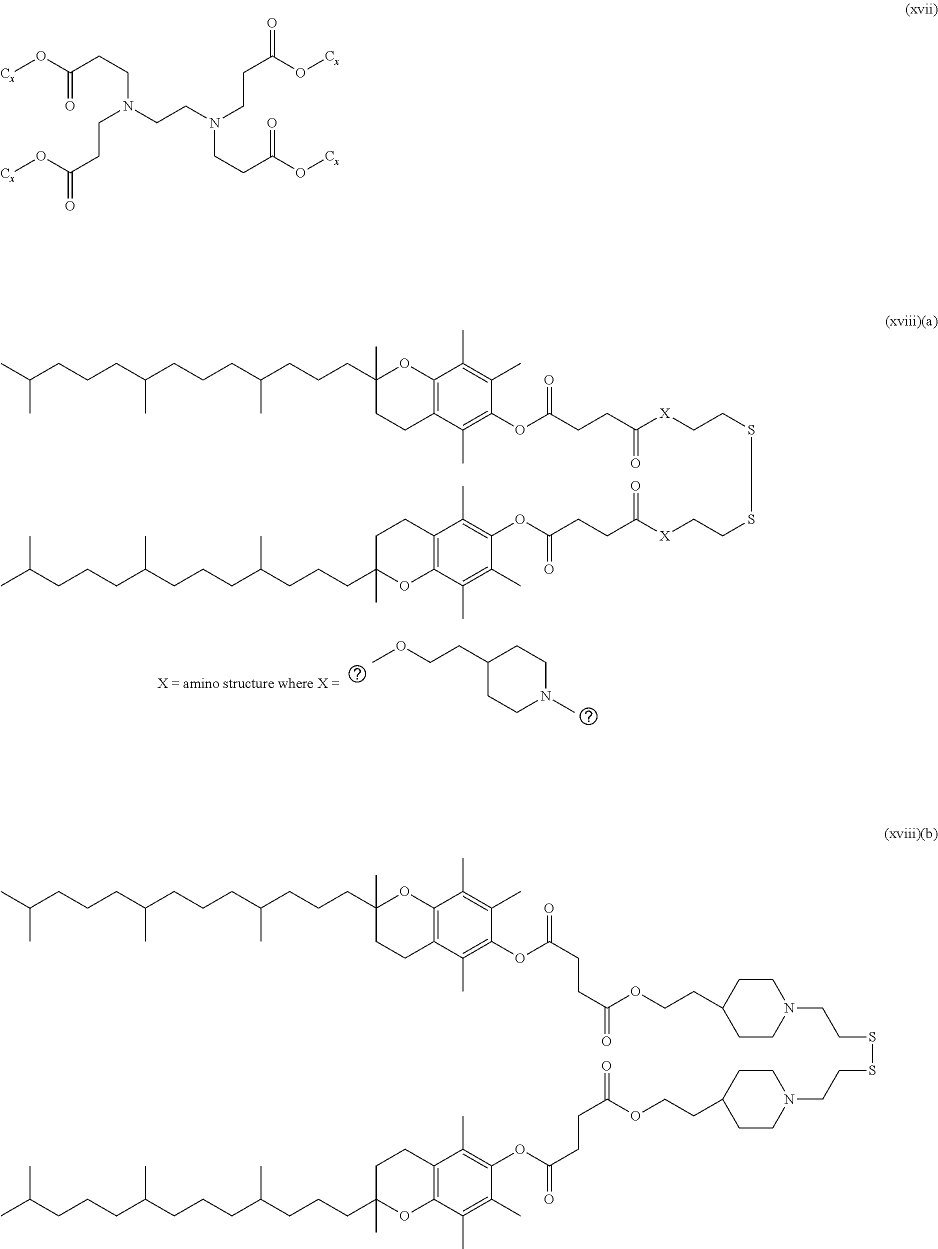

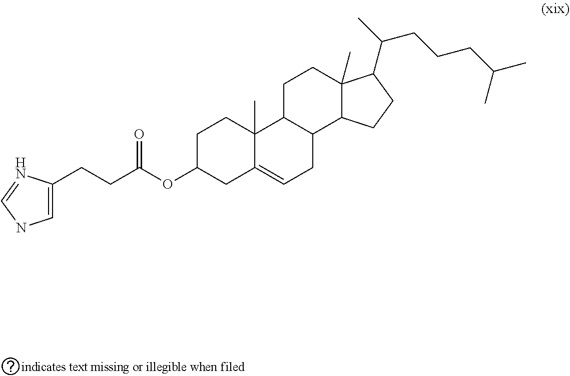

In some embodiments, a lipid compound used to form lipid nanoparticles for the delivery of compositions described herein, e.g., a TREM described herein is made by one of the following reactions:

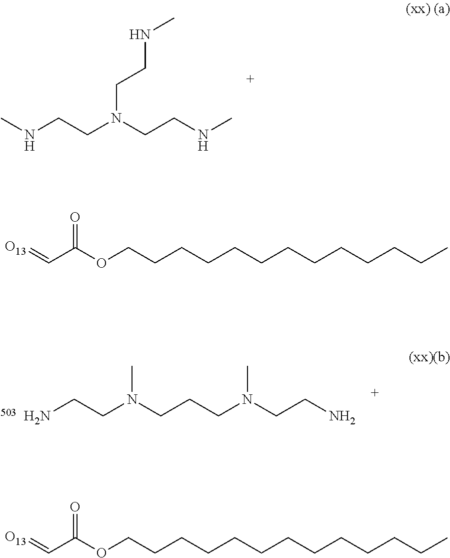

In some embodiments, a composition described herein (e.g., TREM composition) is provided in an LNP that comprises an ionizable lipid. In some embodiments, the ionizable lipid is heptadecan-9-yl 8-((2-hydroxyethyl) (6-oxo-6-(undecyloxy) hexyl)amino) octanoate (SM-102); e.g., as described in Example 1 of U.S. Pat. No. 9,867,888 (incorporated by reference herein in its entirety). In some embodiments, the ionizable lipid is 9Z,12Z)-3-((4,4-bis (octyloxy) butanoyl)oxy)-2-((((3-(diethylamino) propoxy) carbonyl)oxy)methyl) propyl octadeca-9,12-dienoate (LP01), e.g., as synthesized in Example 13 of WO2015/095340 (incorporated by reference herein in its entirety). In some embodiments, the ionizable lipid is Di ((Z)-non-2-en-1-yl) 9-((4-dimethylamino)-butanoyl)oxy) heptadecanedioate (L319), e.g. as synthesized in Example 7, 8, or 9 of US2012/0027803 (incorporated by reference herein in its entirety). In some embodiments, the ionizable lipid is 1,1′-((2-(4-(2-((2-(Bis (2-hydroxydodecyl)amino)ethyl) (2-hydroxydodecyl) amino)ethyl) piperazin-1-yl)ethyl) azanediyl)bis (dodecan-2-ol) (C12-200), e.g., as synthesized in Examples 14 and 16 of WO2010/053572 (incorporated by reference herein in its entirety). In some embodiments, the ionizable lipid is Imidazole cholesterol ester (ICE) lipid (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-IH-cyclopenta[a] phenanthren-3-yl 3-(1H-imidazol-4-yl) propanoate, e.g., Structure (I) from WO2020/106946 (incorporated by reference herein in its entirety).

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle comprises a cationic lipid in formulation with one or more of neutral lipids, ionizable amine-containing lipids, biodegradable alkyne lipids, steroids, phospholipids including polyunsaturated lipids, structural lipids (e.g., sterols), PEG, cholesterol and polymer conjugated lipids. In some embodiments, the cationic lipid may be an ionizable cationic lipid. An exemplary cationic lipid as disclosed herein may have an effective pKa over 6.0. In embodiments, a lipid nanoparticle may comprise a second cationic lipid having a different effective pKa (e.g., greater than the first effective pKa), than the first cationic lipid. A lipid nanoparticle may comprise between 40 and 60 mol percent of a cationic lipid, a neutral lipid, a steroid, a polymer conjugated lipid, and a therapeutic agent, e.g., a TREM described herein, encapsulated within or associated with the lipid nanoparticle. In some embodiments, the TREM is co-formulated with the cationic lipid. The TREM may be adsorbed to the surface of an LNP, e.g., an LNP comprising a cationic lipid. In some embodiments, the TREM may be encapsulated in an LNP, e.g., an LNP comprising a cationic lipid. In some embodiments, the lipid nanoparticle may comprise a targeting moiety, e.g., coated with a targeting agent. In embodiments, the LNP formulation is biodegradable. In some embodiments, a lipid nanoparticle comprising one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of a TREM.

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or III of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; I, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; I, II, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; III-3 of WO2018/081480; I-5 or I-8 of WO2020/081938; 18 or 25 of U.S. Pat. No. 9,867,888; A of US2019/0136231; II of WO2020/219876; 1 of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-013 or 503-013 of Whitehead et al; TS-P4C2 of U.S. Pat. No. 9,708,628; I of WO2020/106946; I of WO2020/106946.

In some embodiments, the ionizable lipid is MC3 (6Z,9Z,28Z.3 IZ)-heptatriaconta-6,9,28,3 1-tetraen-19-yl-4-(dimethylamino) butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (incorporated by reference herein in its entirety). In some embodiments, the ionizable lipid is (13Z,16Z)-A,A-dimethyl-3-nonyldocosa-13, 16-dien-1-amine (Compound 32), e.g., as described in Example 11 of WO2019051289A9 (incorporated by reference herein in its entirety). In some embodiments, the ionizable lipid is Compound 6 or Compound 22, e.g., as described in Example 12 of WO2019051289A9 (incorporated by reference herein in its entirety).

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), diolcoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, paimitoyl, stearoyl, or olcoyl. Additional exemplary lipids, in certain embodiments, include, without limitation, those described in Kim et al. (2020) dx.doi.org/10.1021/acs.nanolett.0c01386, incorporated herein by reference. Such lipids include, in some embodiments, plant lipids found to improve liver transfection with mRNA (e.g., DGTS).

Other examples of non-cationic lipids suitable for use in the lipid nanoparticles include, without limitation, nonphosphorous lipids such as, e.g., stearylamine, dodecylamine, hexadecylamine, acetyl palmitate, glycerol ricinoleate, hexadecyl stercate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the contents of which is incorporated herein by reference in their entirety.

In some embodiments, the non-cationic lipid is oleic acid or a compound of Formula I, II, or IV of US2018/0028664, incorporated herein by reference in its entirety. The non-cationic lipid can comprise, for example, 0-30% (mol) of the total lipid present in the lipid nanoparticle. In some embodiments, the non-cationic lipid content is 5-20% (mol) or 10-15% (mol) of the total lipid present in the lipid nanoparticle. In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not comprise any phospholipids.

In some aspects, the lipid nanoparticle can further comprise a component, such as a sterol, to provide membrane integrity. One exemplary sterol that can be used in the lipid nanoparticle is cholesterol and derivatives thereof. Non-limiting examples of cholesterol derivatives include polar analogues such as 5a-choiestanol, 53-coprostanol, choiesteryl-(2′-hydroxy)-ethyl ether, choiesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., choiesteryl-(4 '-hydroxy)-butyl ether. Exemplary cholesterol derivatives are described in PCT publication WO2009/127060 and US patent publication US2010/0130588, each of which is incorporated herein by reference in its entirety.

In some embodiments, the component providing membrane integrity, such as a sterol, can comprise 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di (tetradecanoyloxy) propyl-1-O-(w-methoxy (polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, and US/099823, the contents of all of which are incorporated herein by reference in their entirety. In some embodiments, a PEG-lipid is a compound of Formula III, III-a-I, III-a-2, III-b-1, III-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3 [beta]-oxy) carboxamido-3′,6′-dioxaoctanyl] carbamoyl-[omega]-methyl-poly (ethylene glycol), PEG-DMB (3,4-Ditetradecoxylbenzyl-[omega]-methyl-poly (ethylene glycol) ether), and 1,2-dimyristoyl-sn-glycero-3-phosphoethanolamine-N-[methoxy (polyethylene glycol)-2000]. In some embodiments, the PEG-lipid comprises PEG-DMG, 1,2-dimyristoyl-sn-glycero-3-phosphoethanolamine-N-[methoxy (polyethylene glycol)-2000]. In some embodiments, the PEG-lipid comprises a structure selected from:

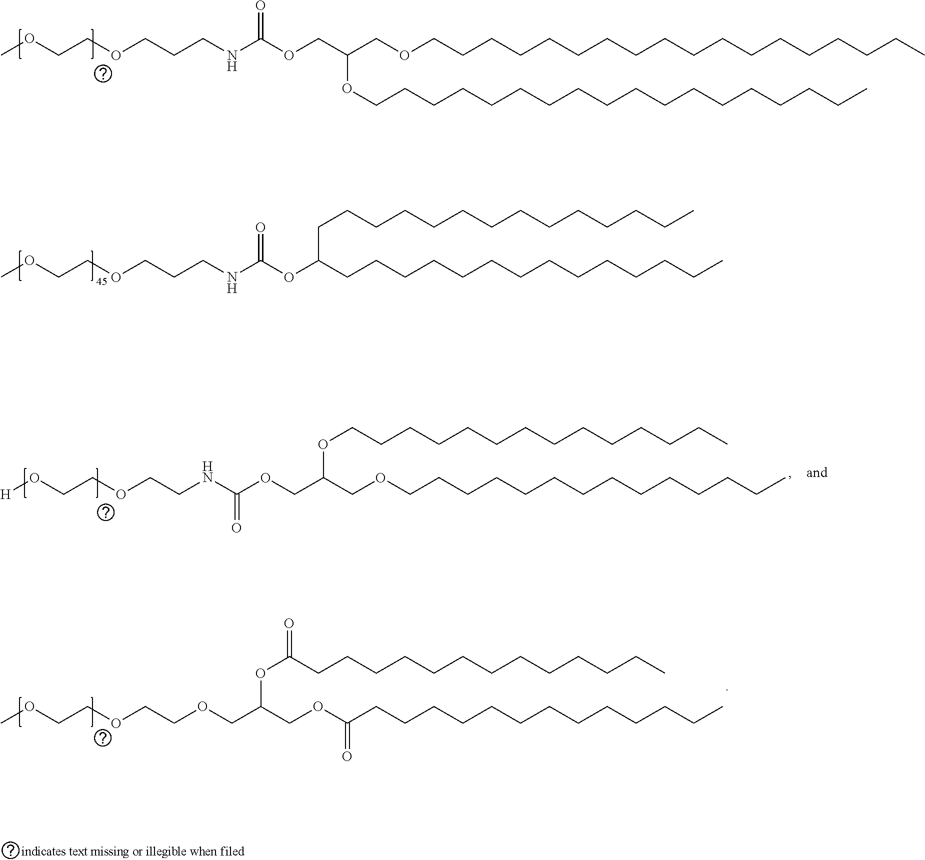

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and LIS patent applications listed in Table 2 of WO2019051289A9, the contents of all of which are incorporated herein by reference in their entirety.

In some embodiments, the PEG or the conjugated lipid can comprise 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can comprise 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition comprises 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example comprising 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation comprises ionizable lipid, phospholipid, cholesterol and a PEG-ylated lipid in a molar ratio of 50:10:38.5:1. 5. In some other embodiments, the lipid particle formulation comprises ionizable lipid, cholesterol and a PEG-ylated lipid in a molar ratio of 60:38.5:1.5.

In some embodiments, the lipid particle comprises ionizable lipid, non-cationic lipid (e.g. phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle comprises ionizable lipid/non-cationic-lipid/sterol/conjugated lipid at a molar ratio of 50:10:38.5:1.5.

In an aspect, the disclosure provides a lipid nanoparticle formulation comprising phospholipids, lecithin, phosphatidylcholine and phosphatidylethanolamine.

In some embodiments, one or more additional compounds can also be included. Those compounds can be administered separately, or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

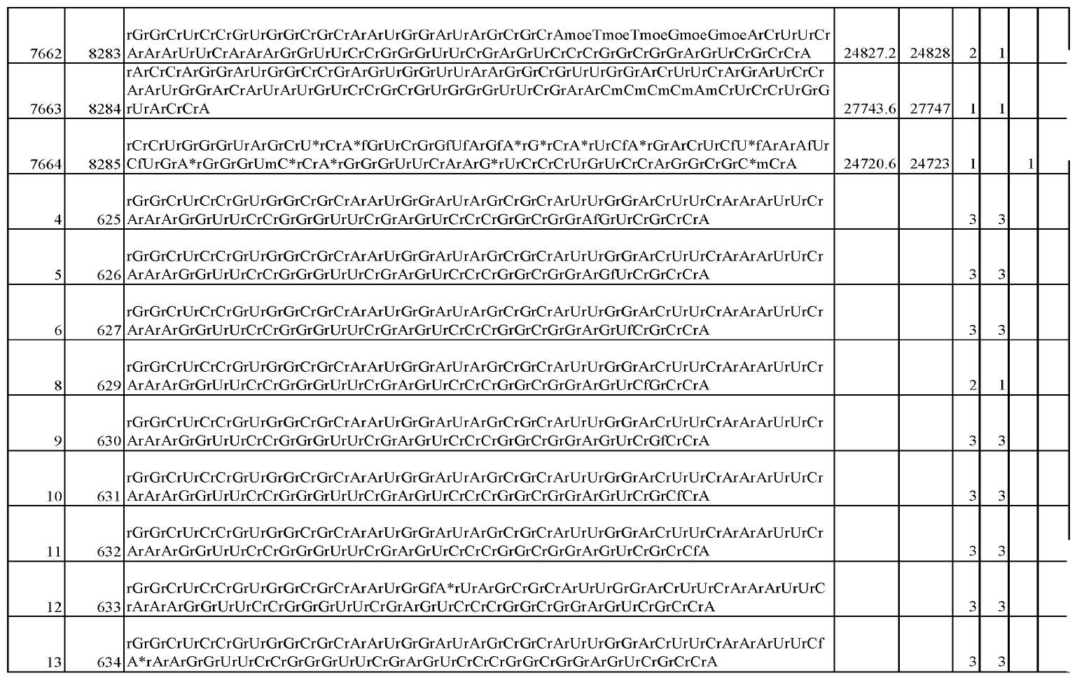

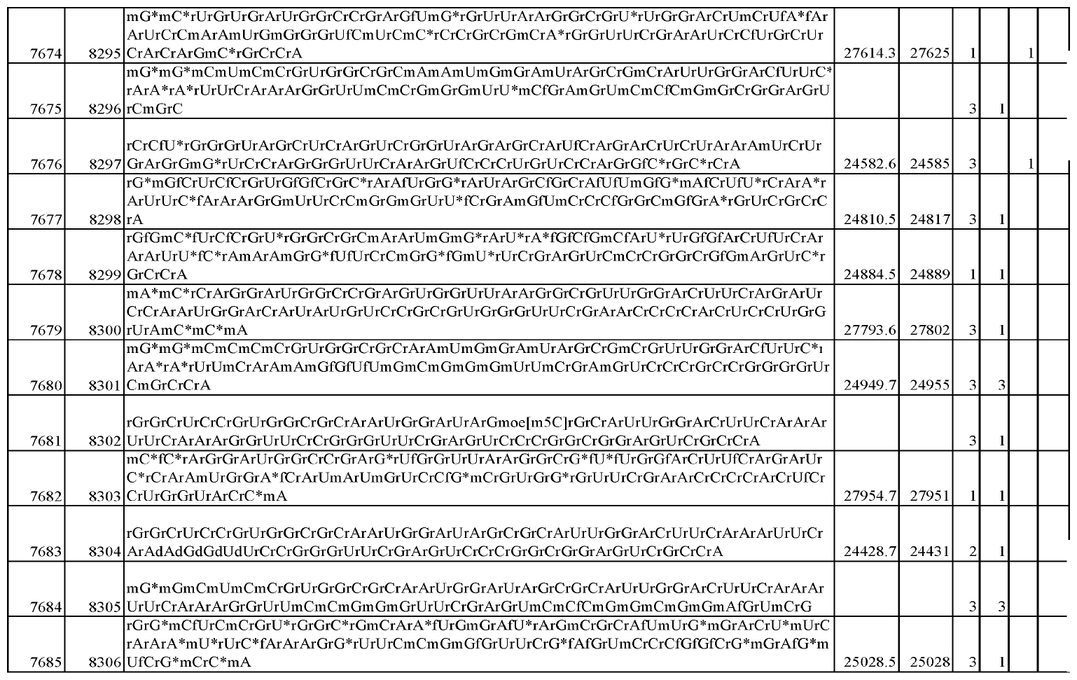

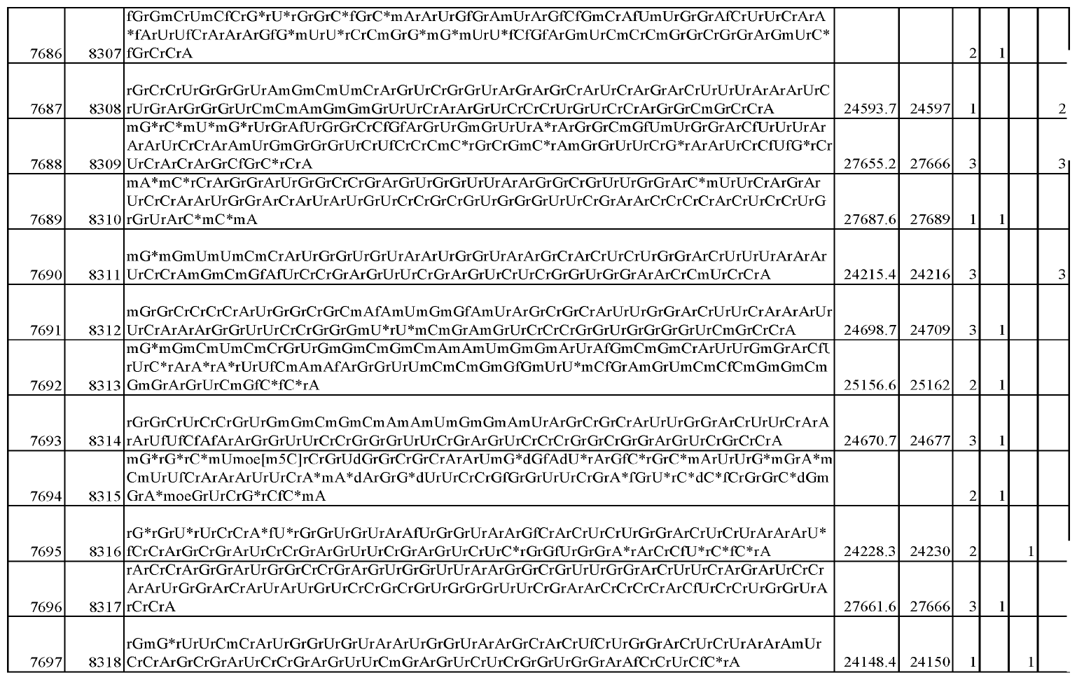

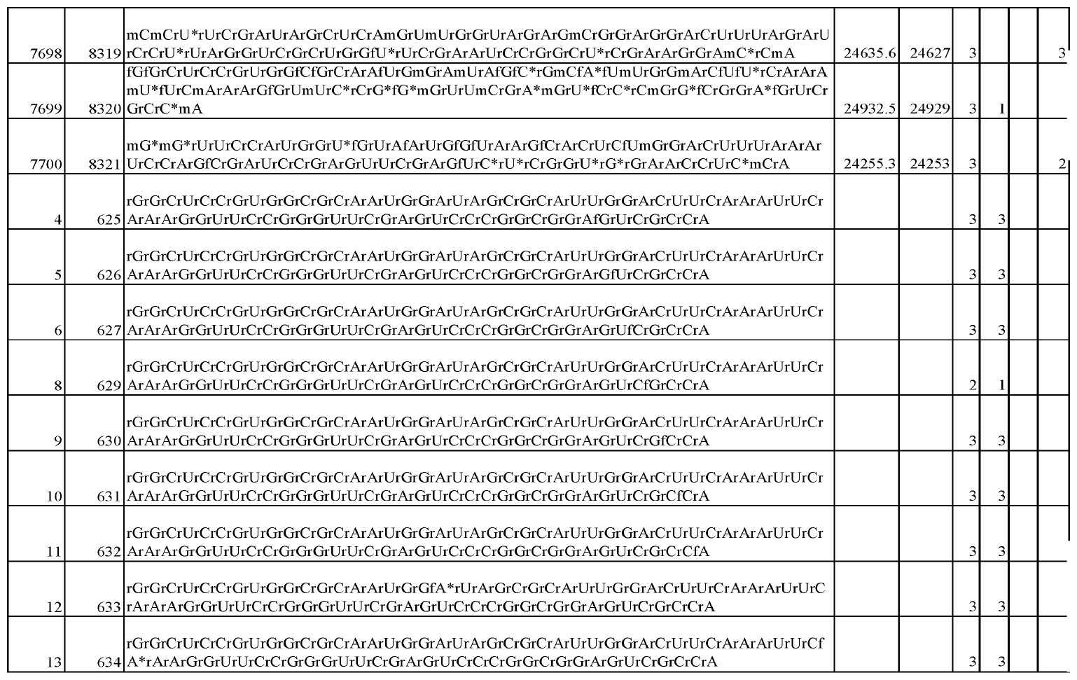

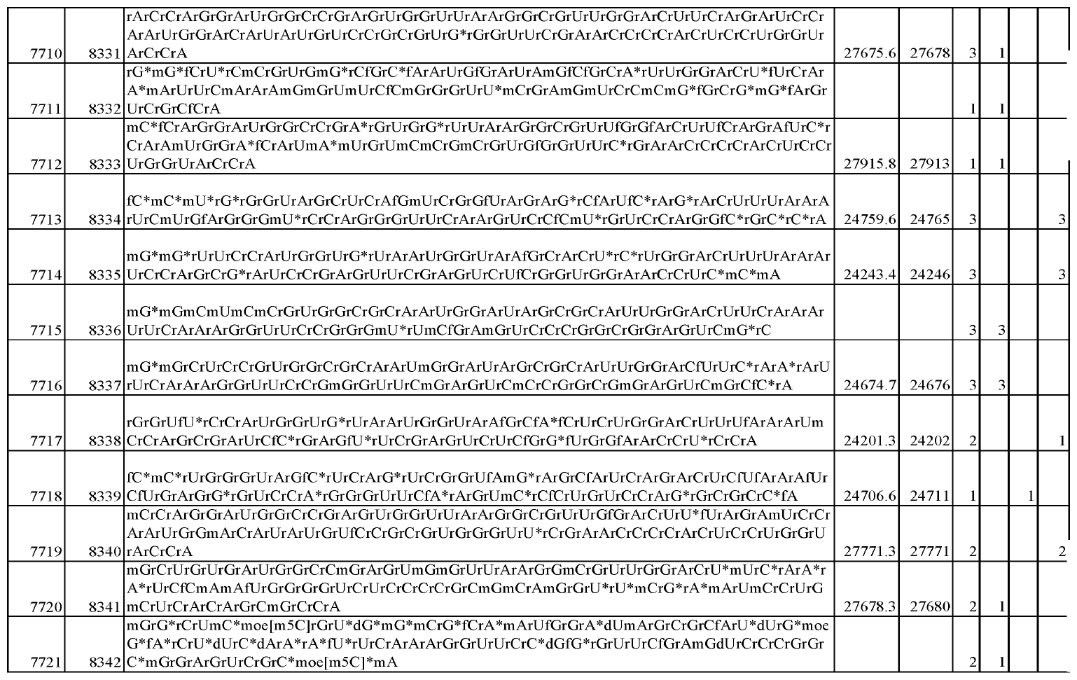

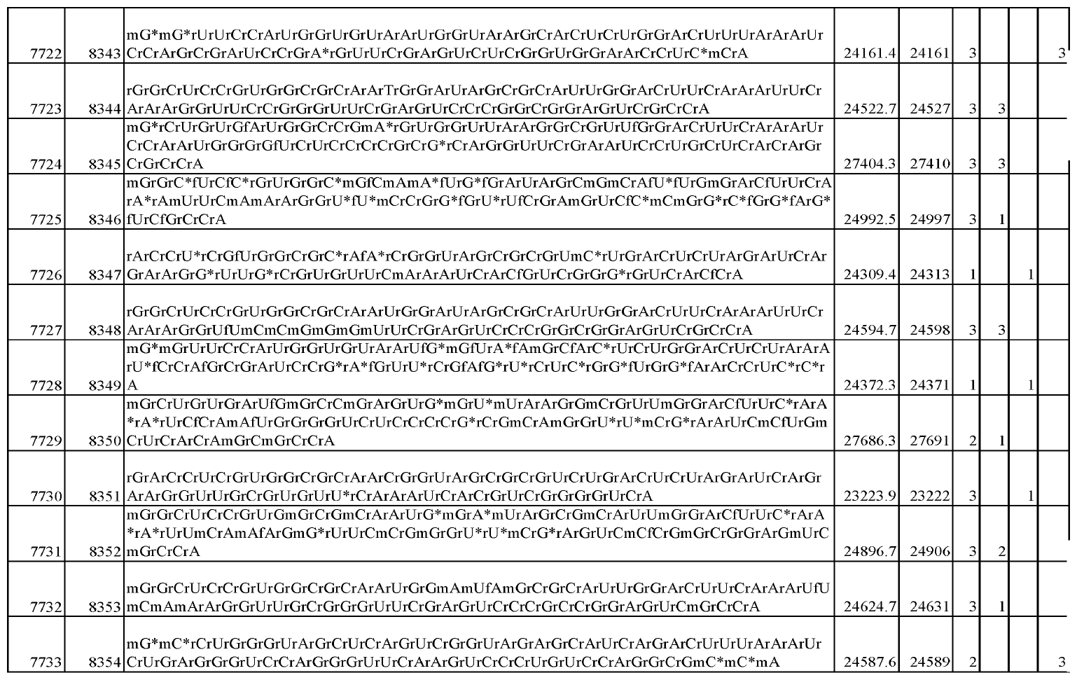

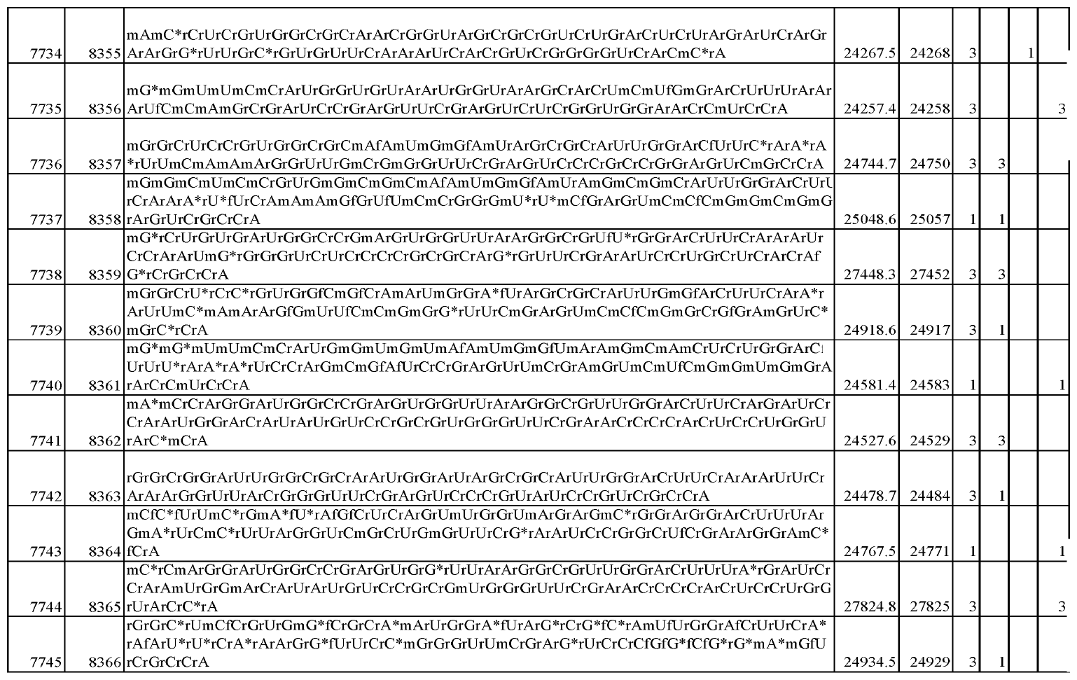

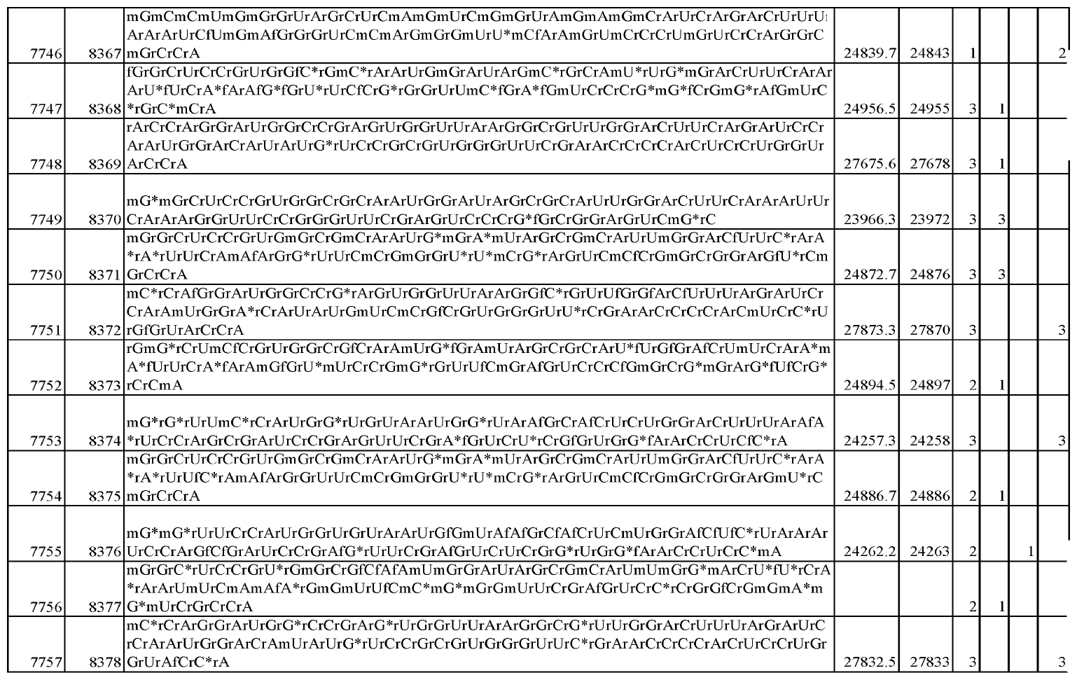

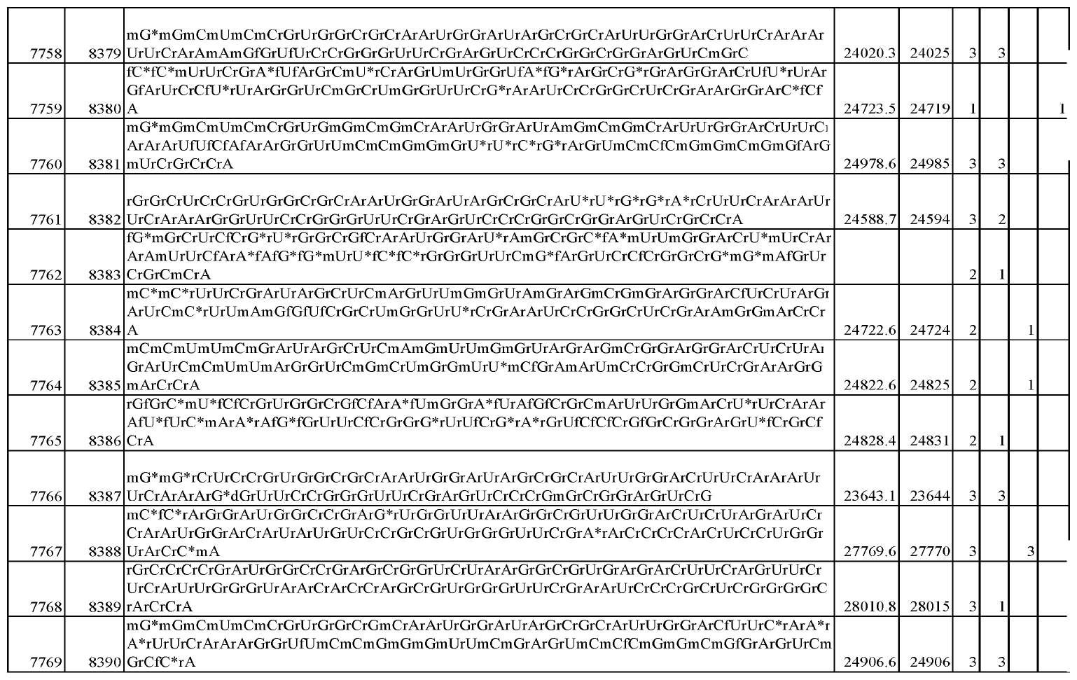

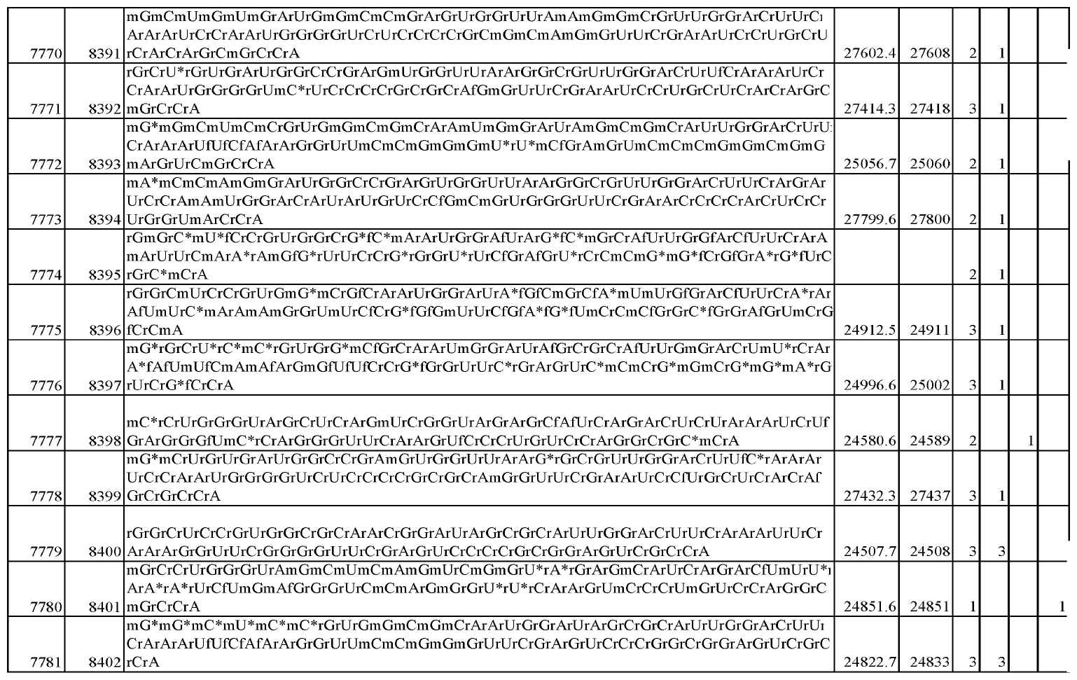

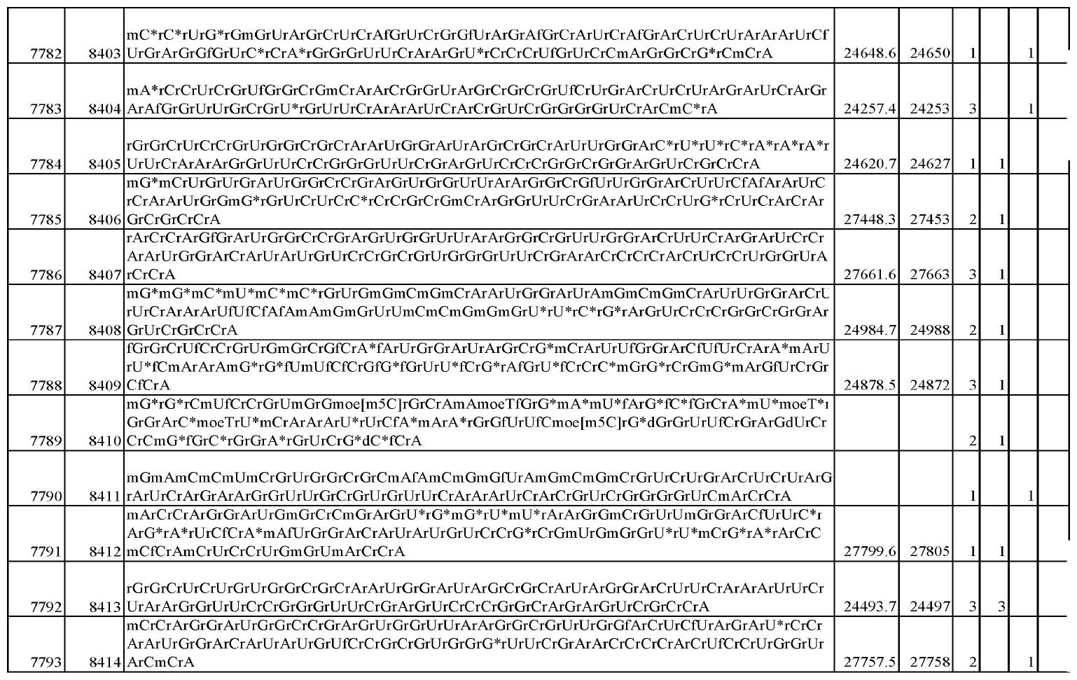

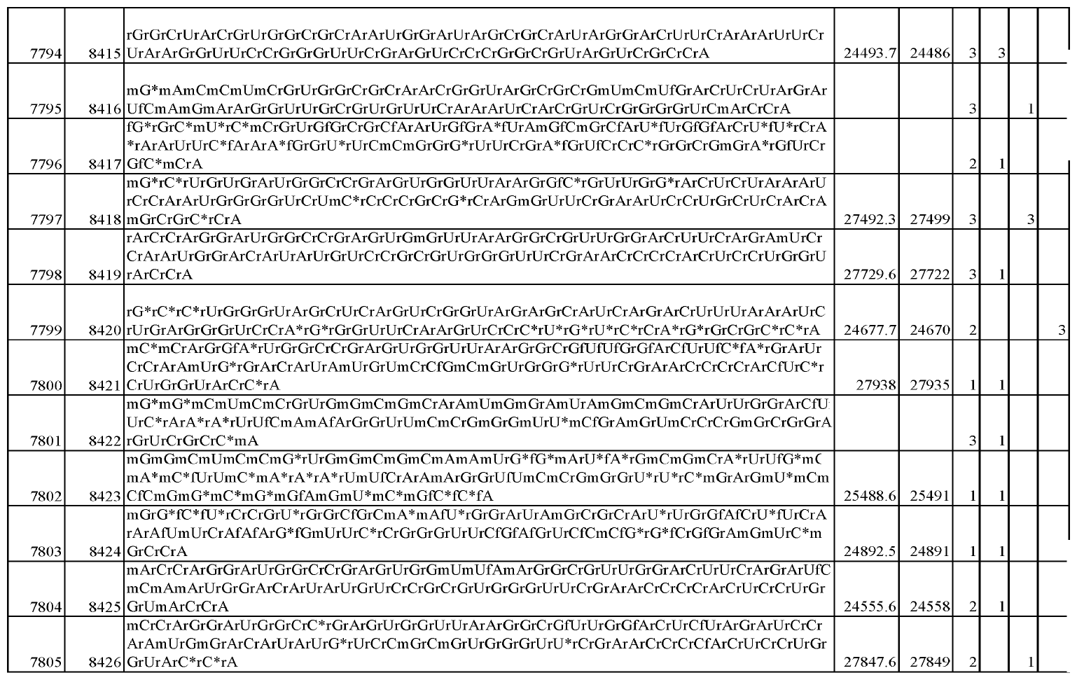

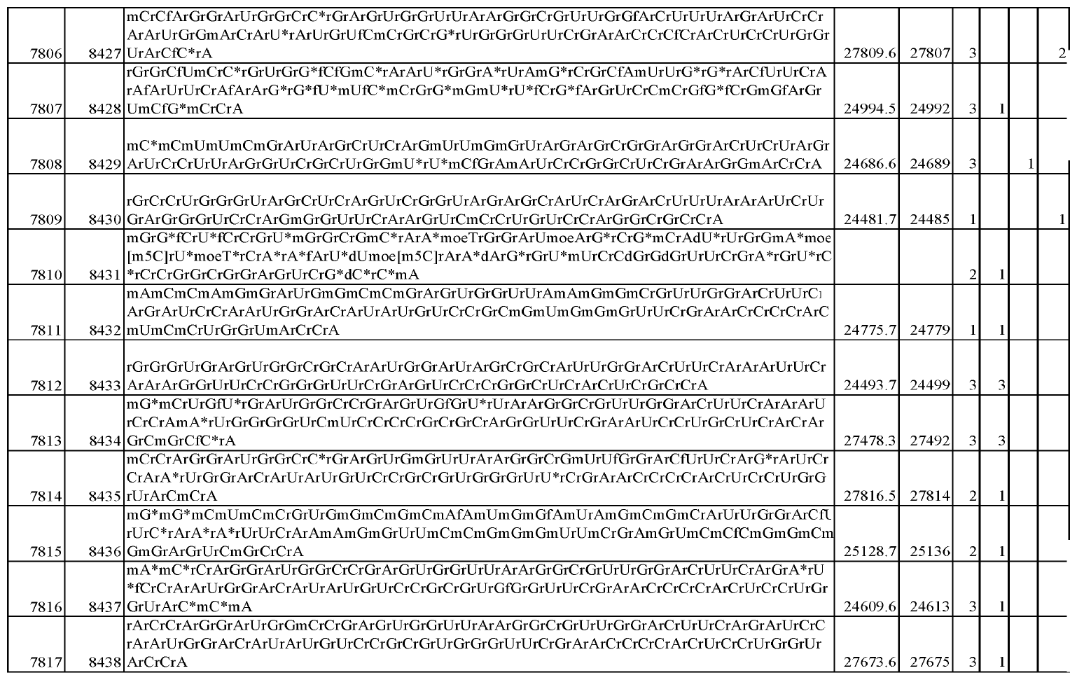

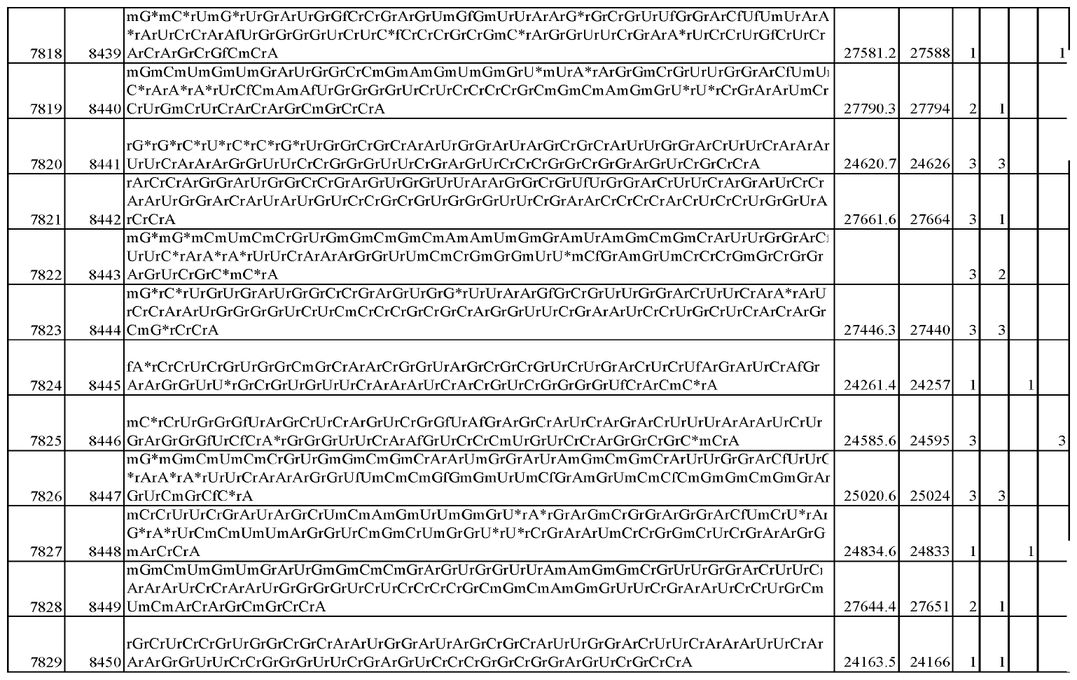

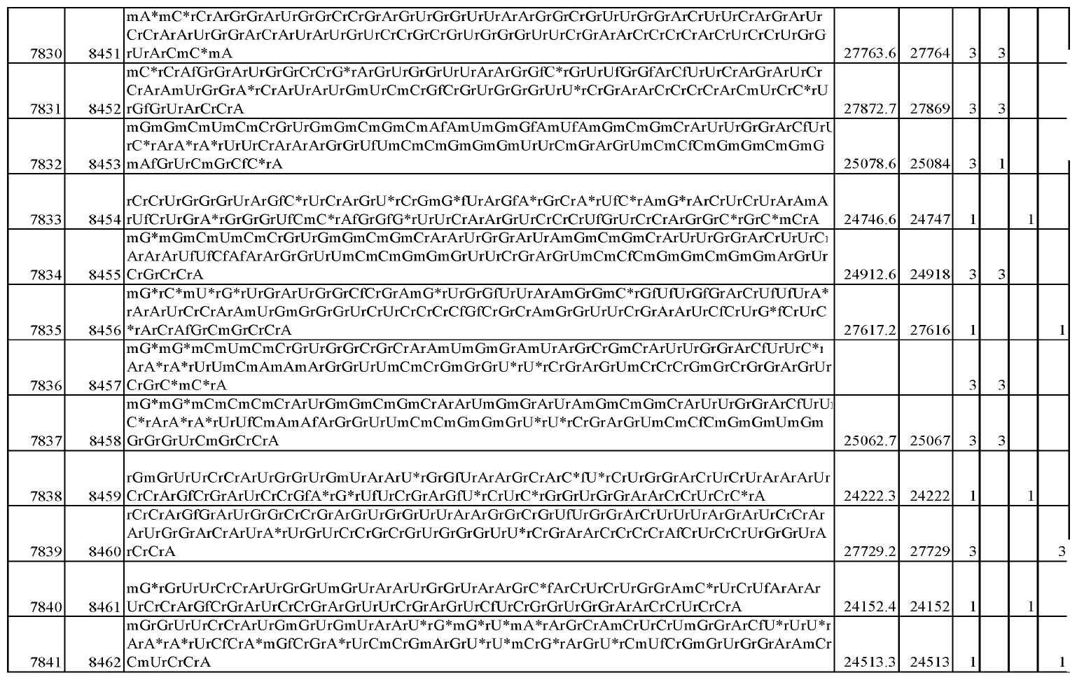

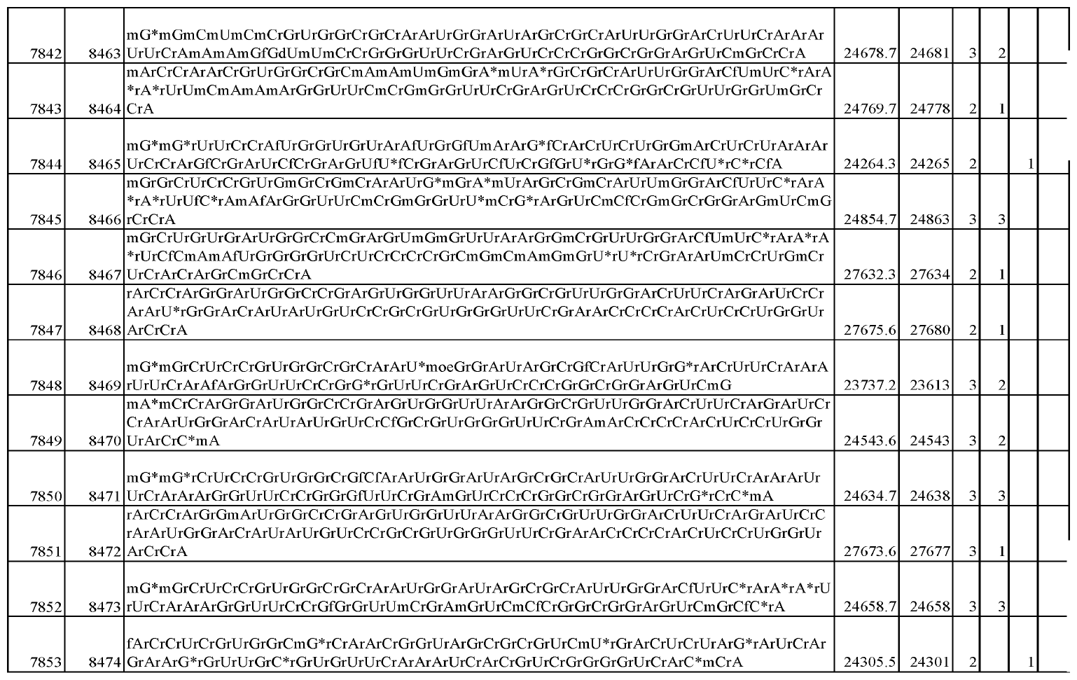

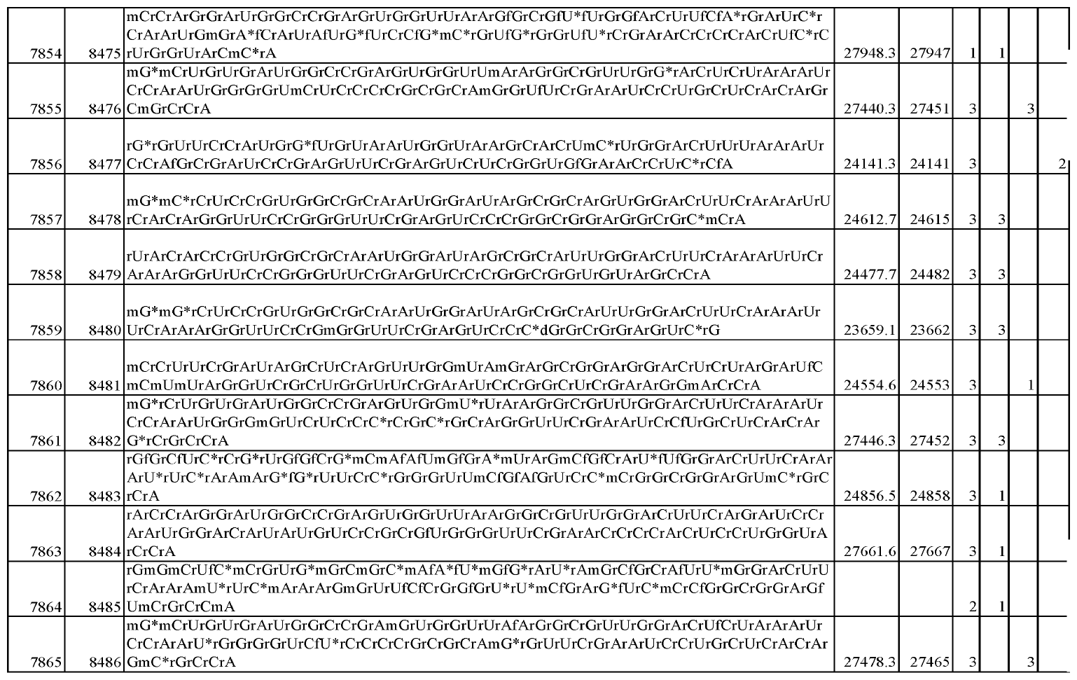

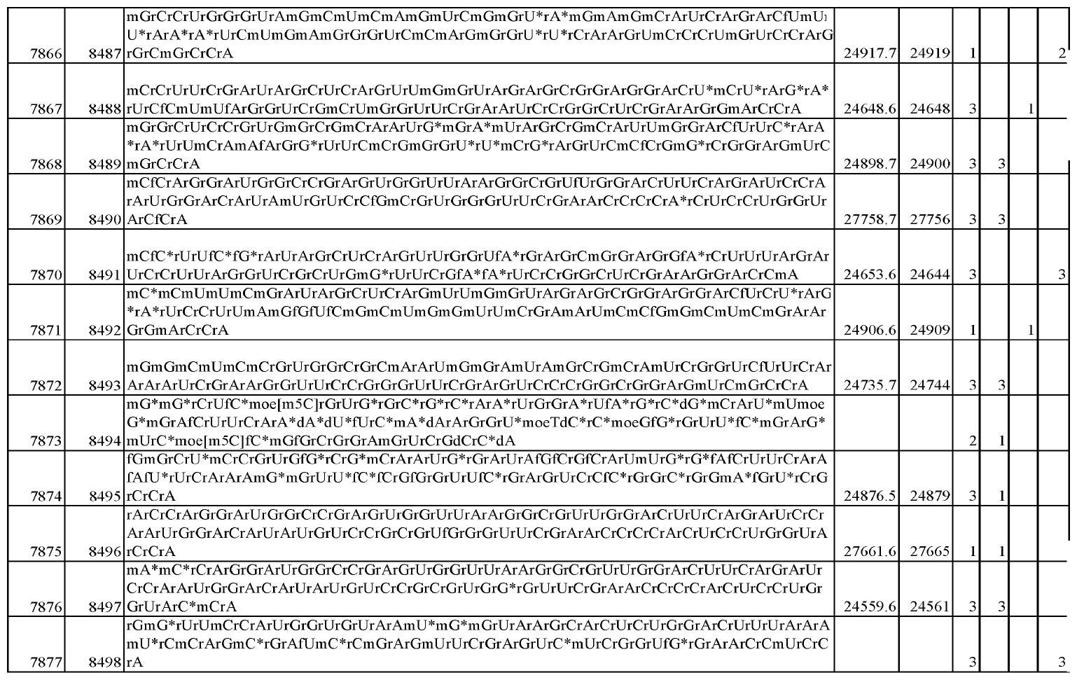

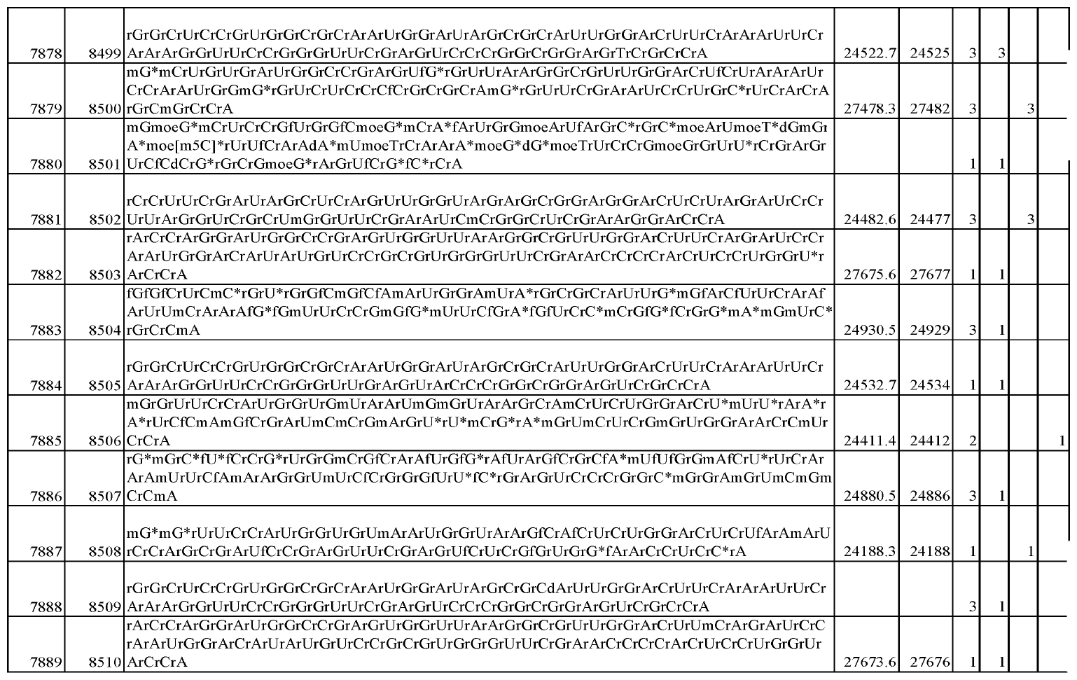

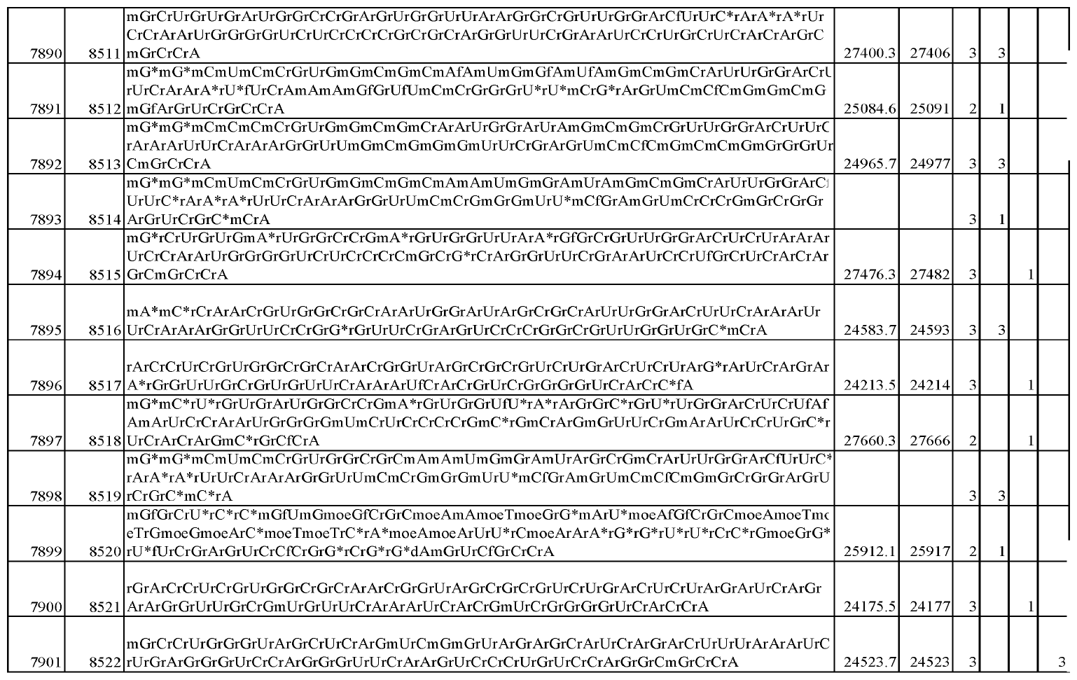

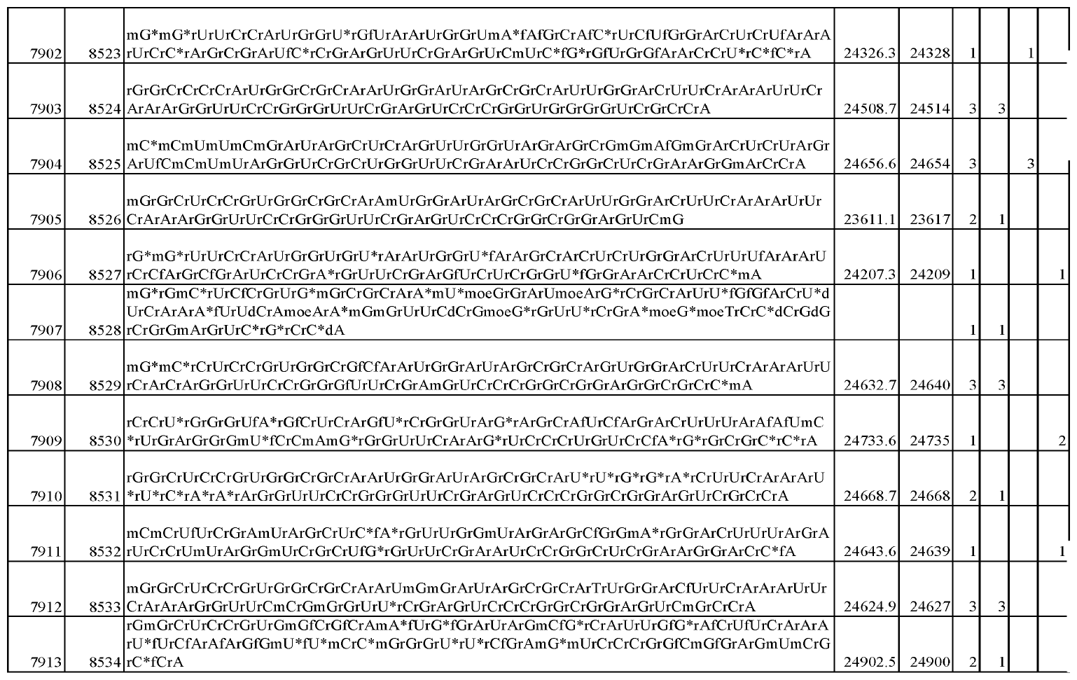

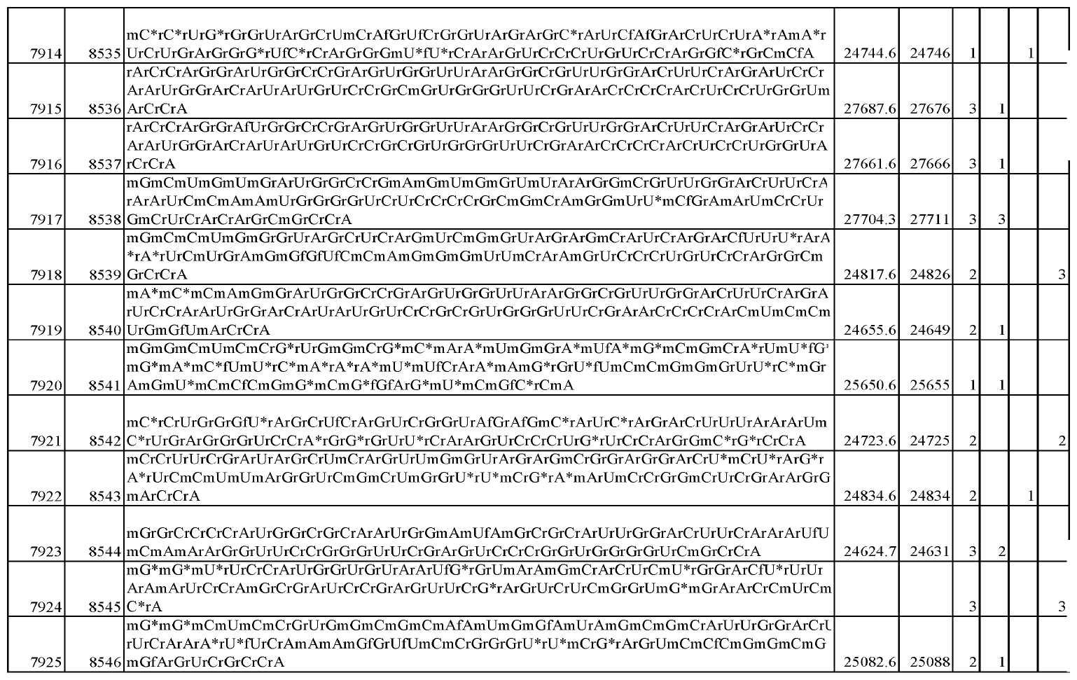

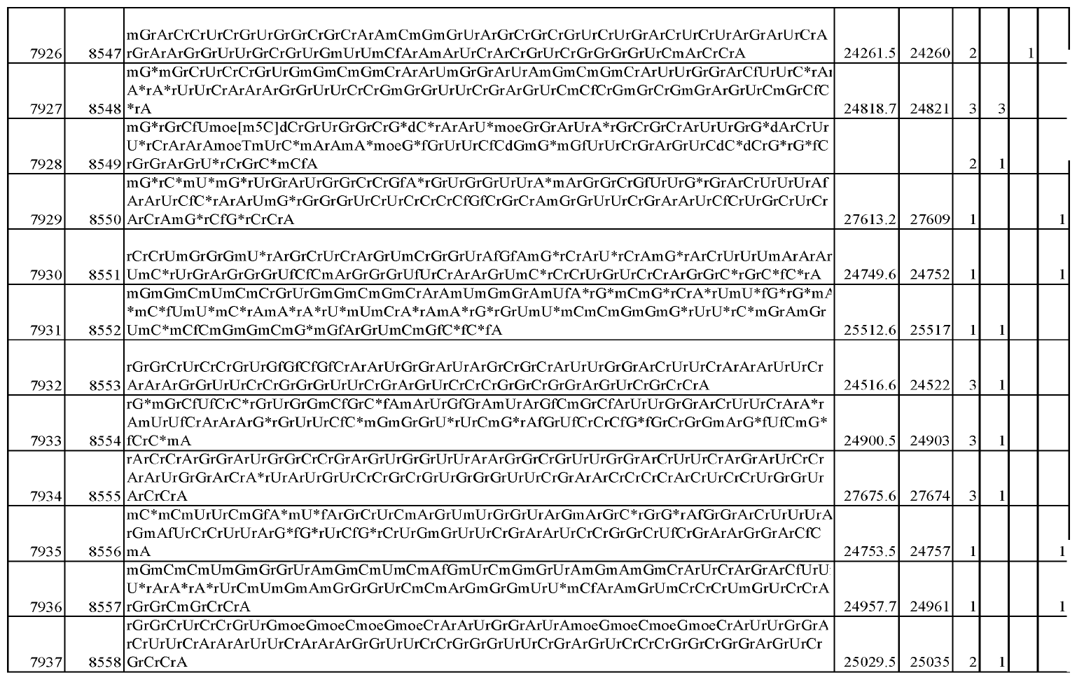

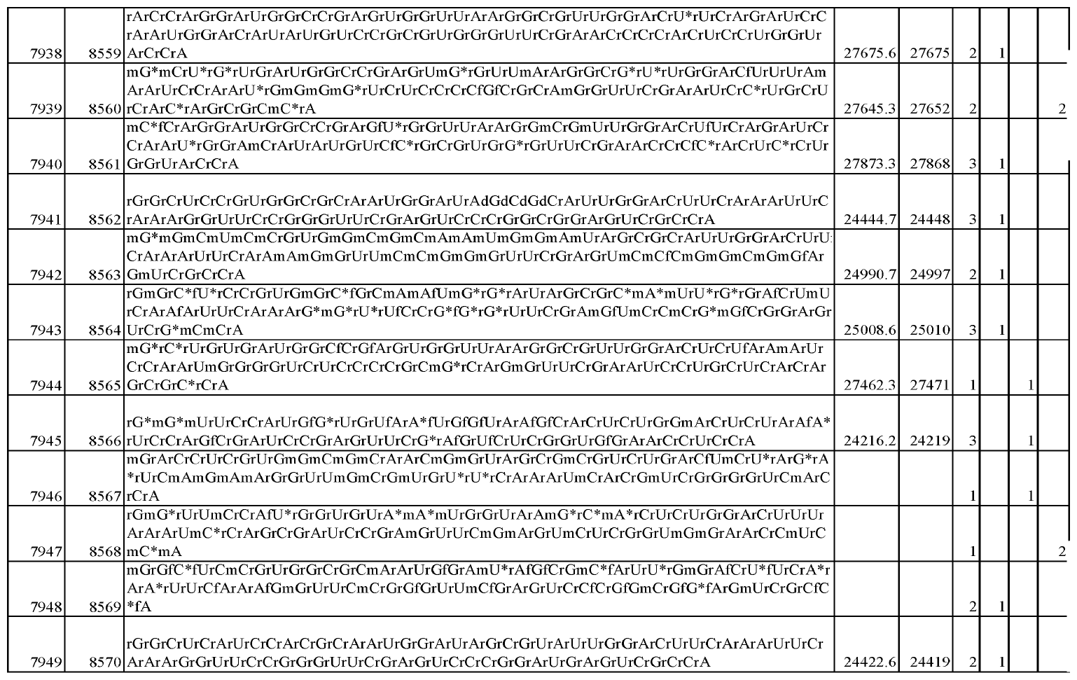

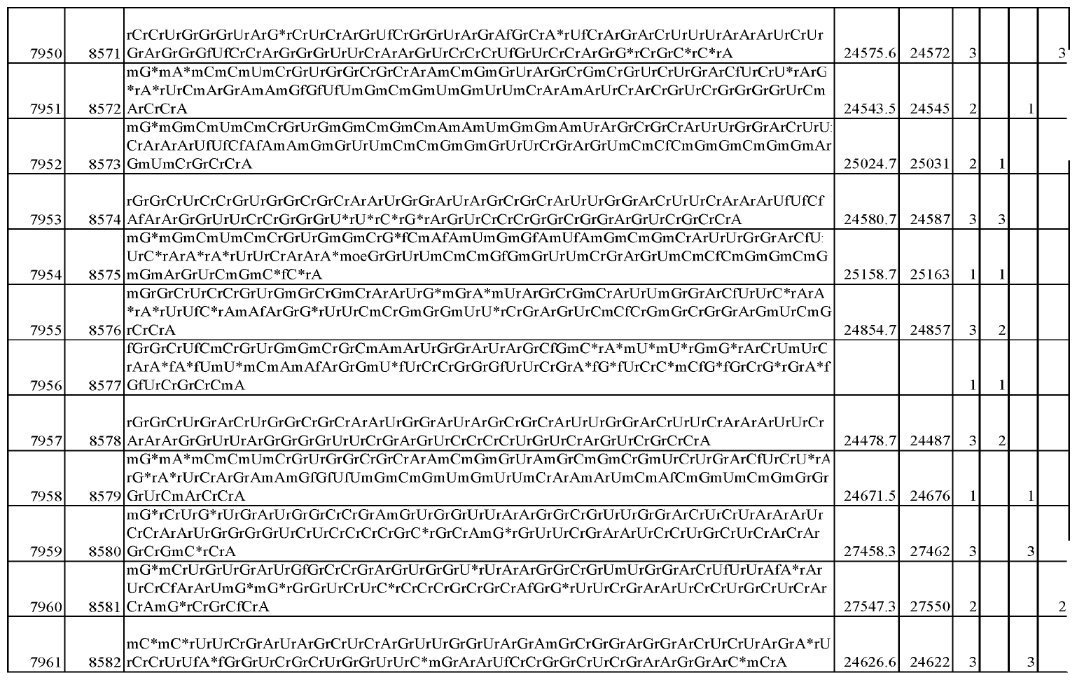

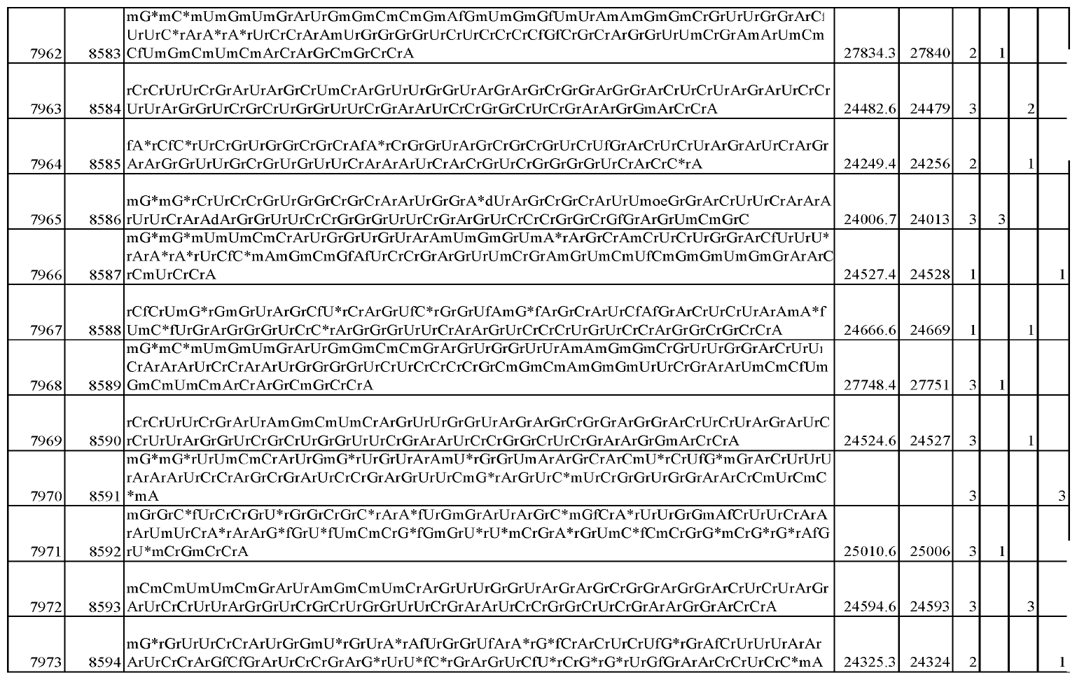

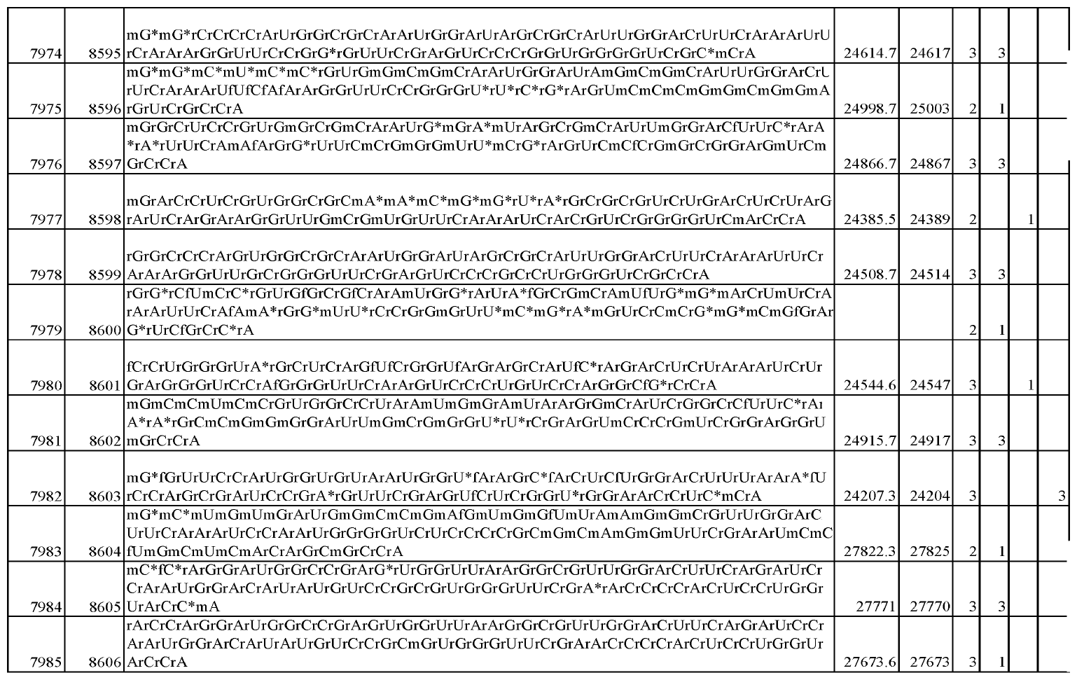

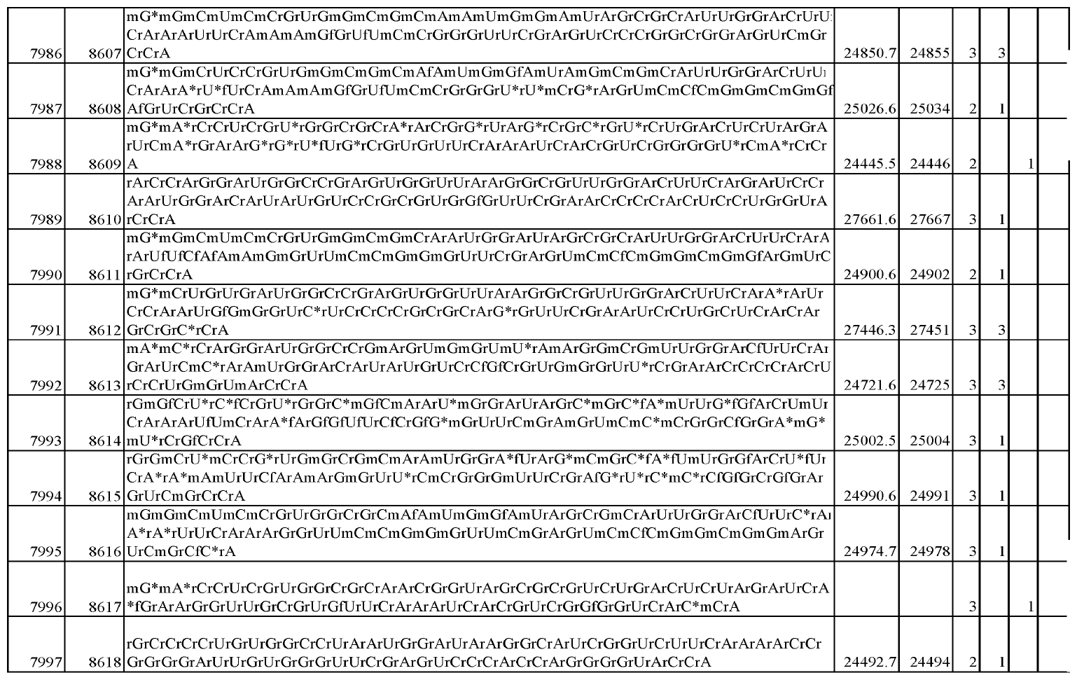

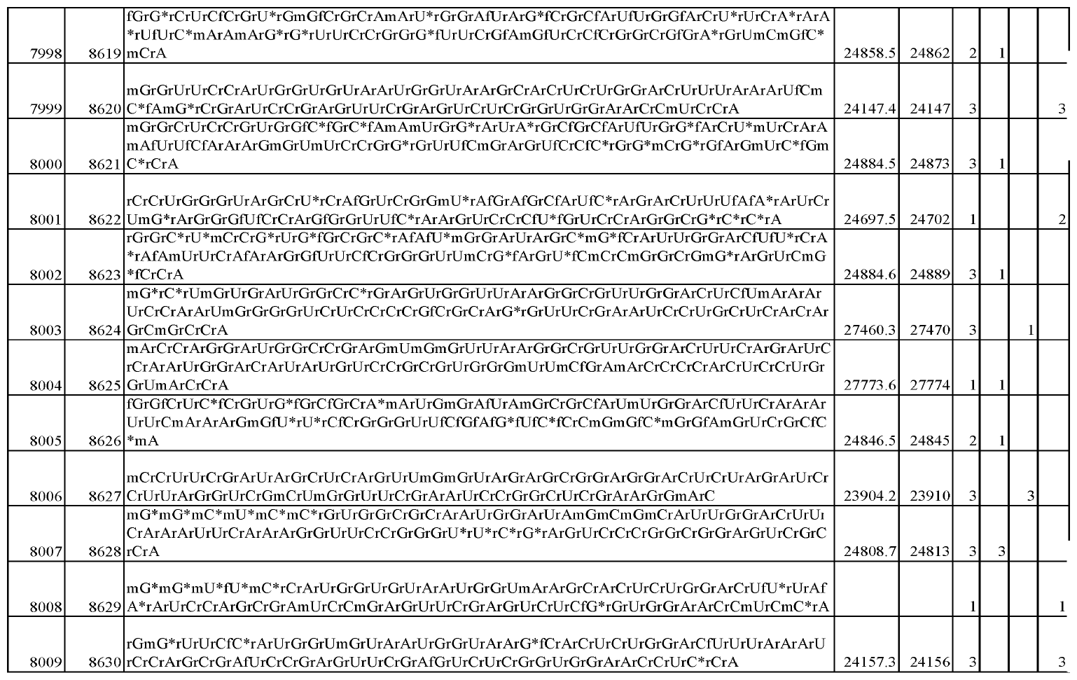

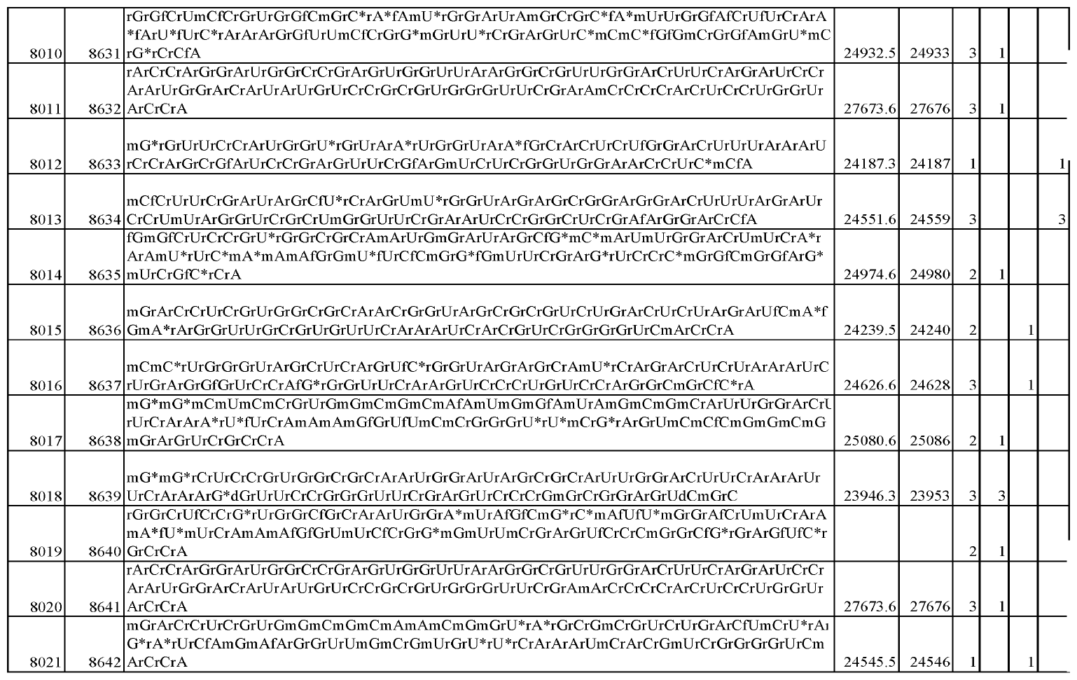

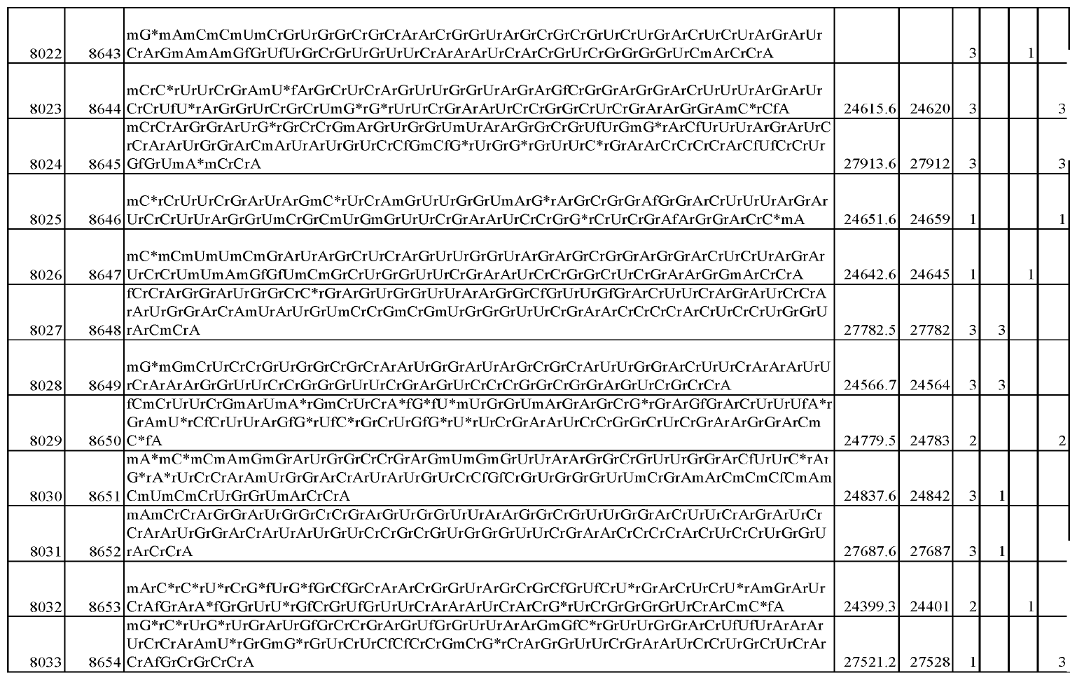

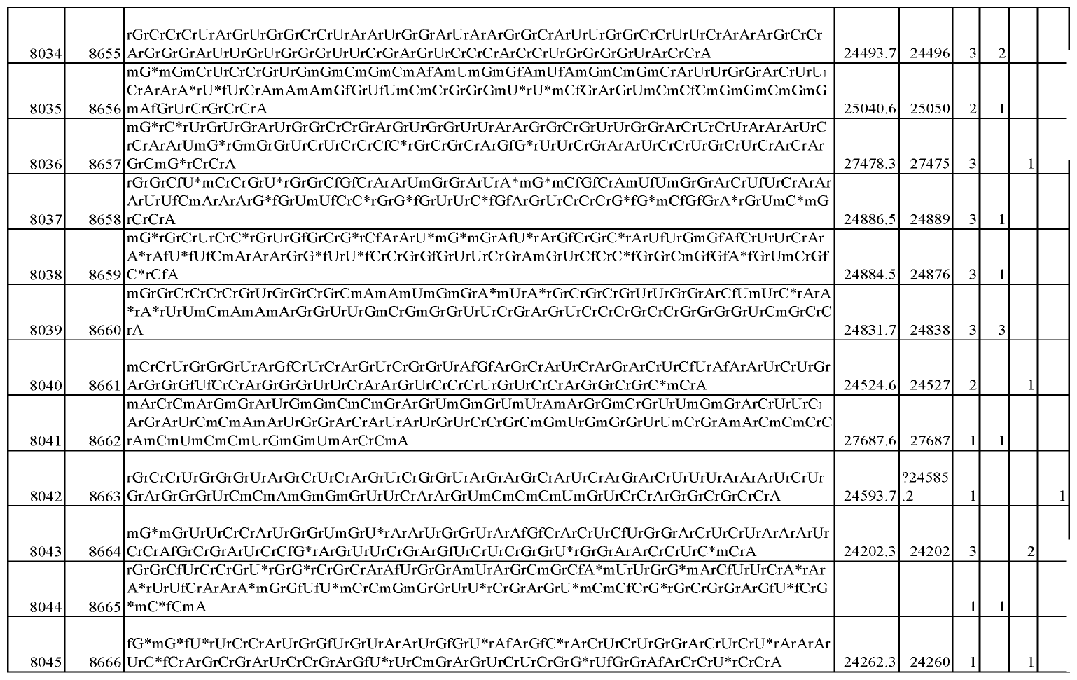

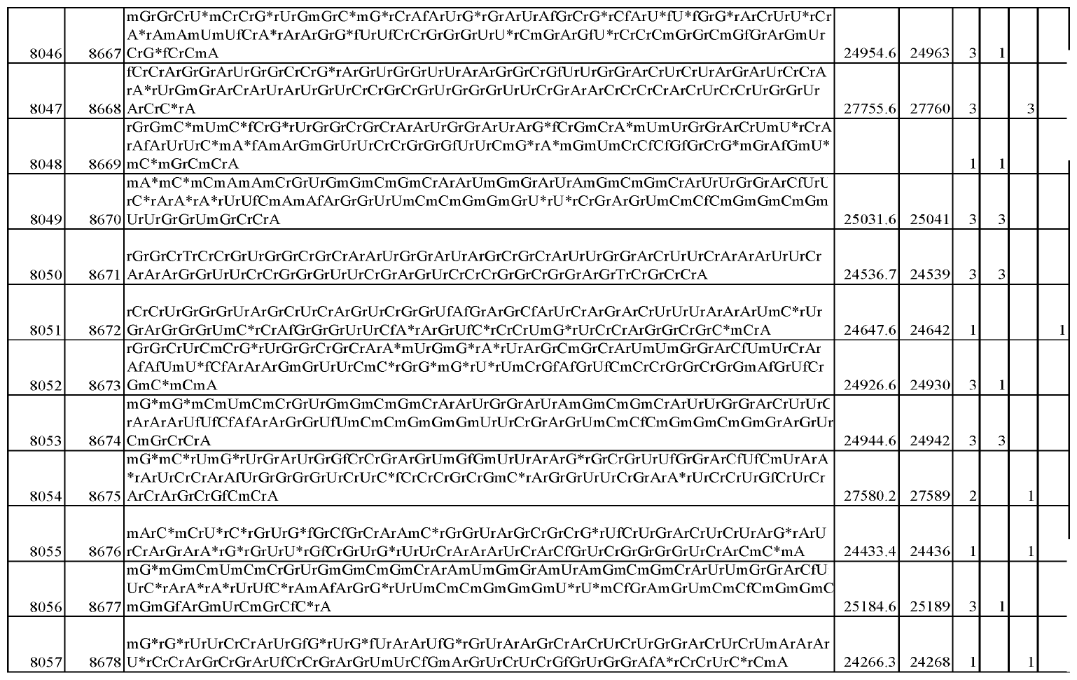

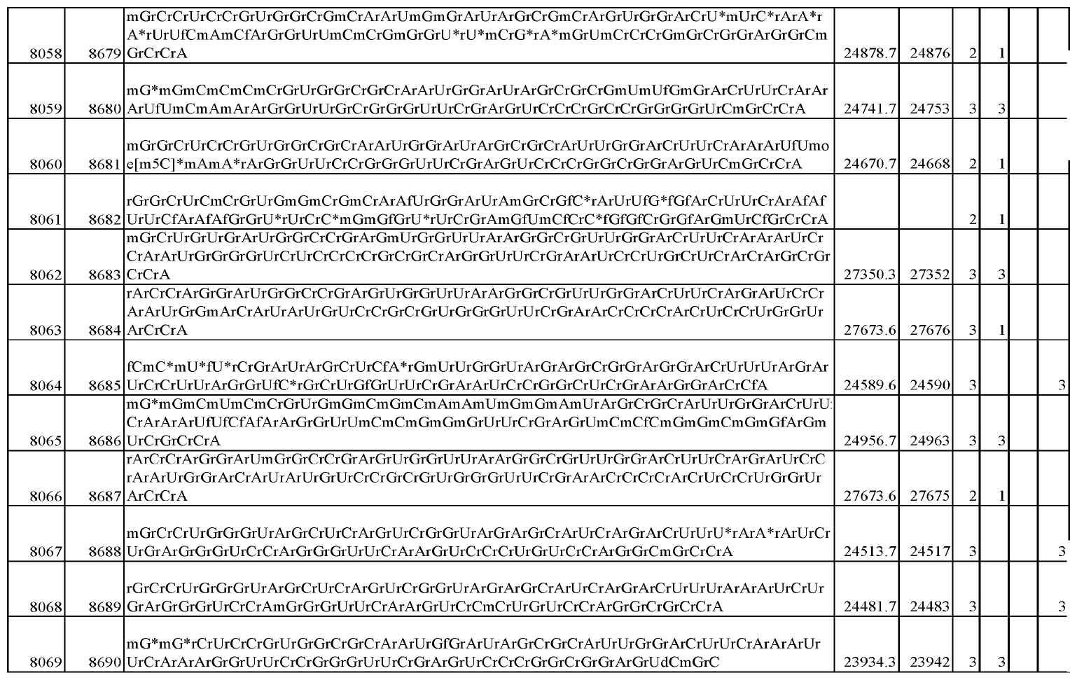

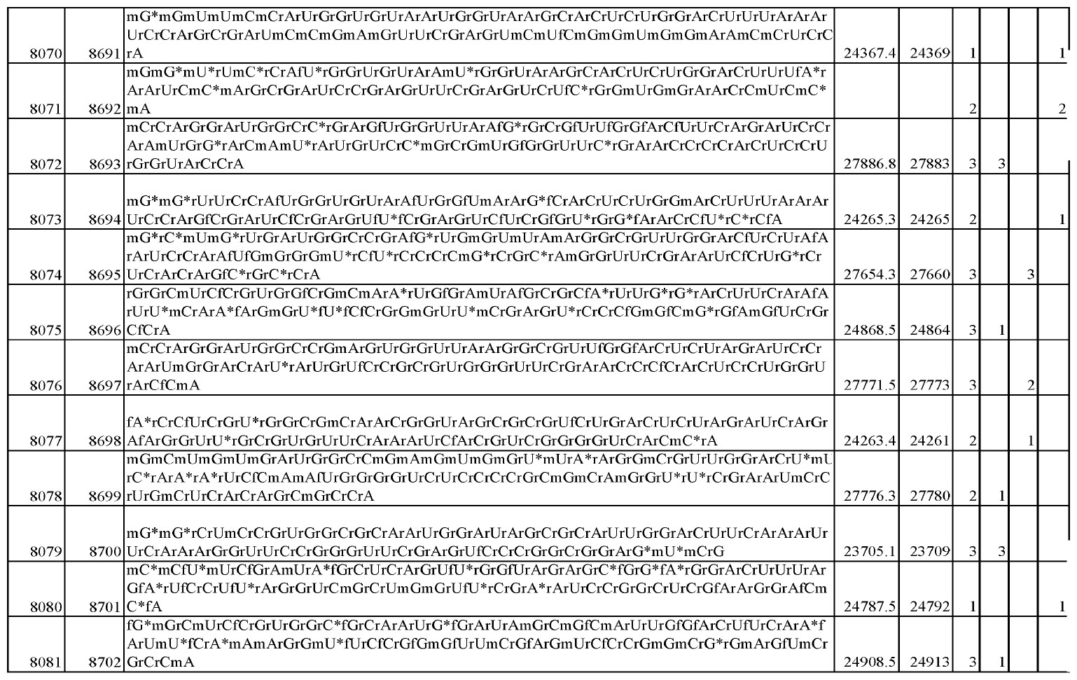

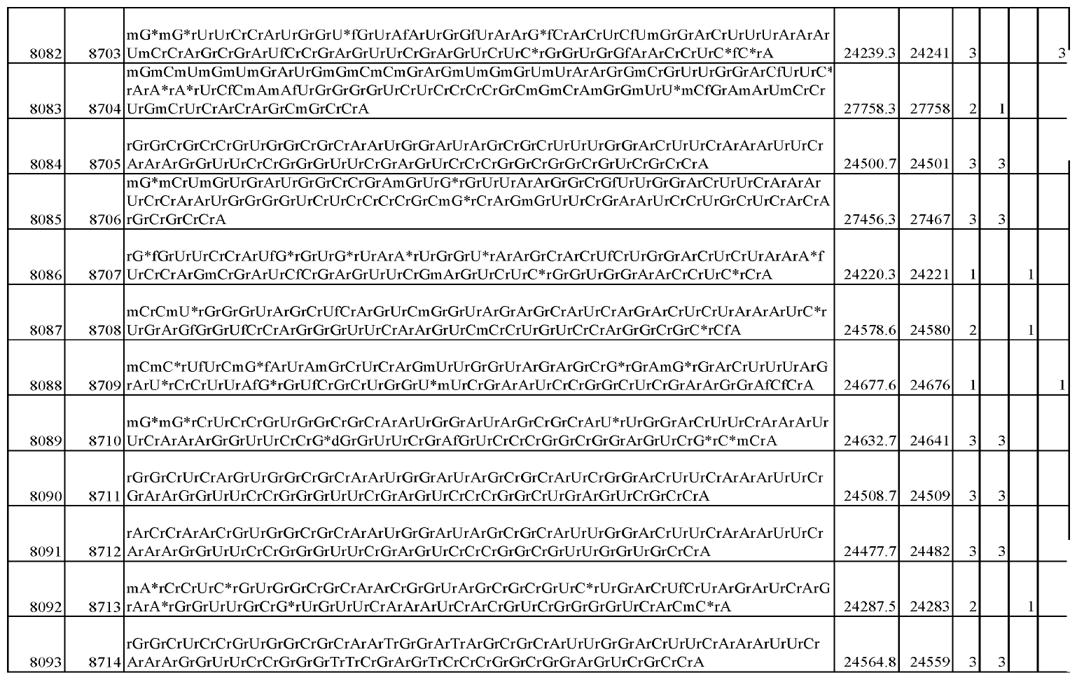

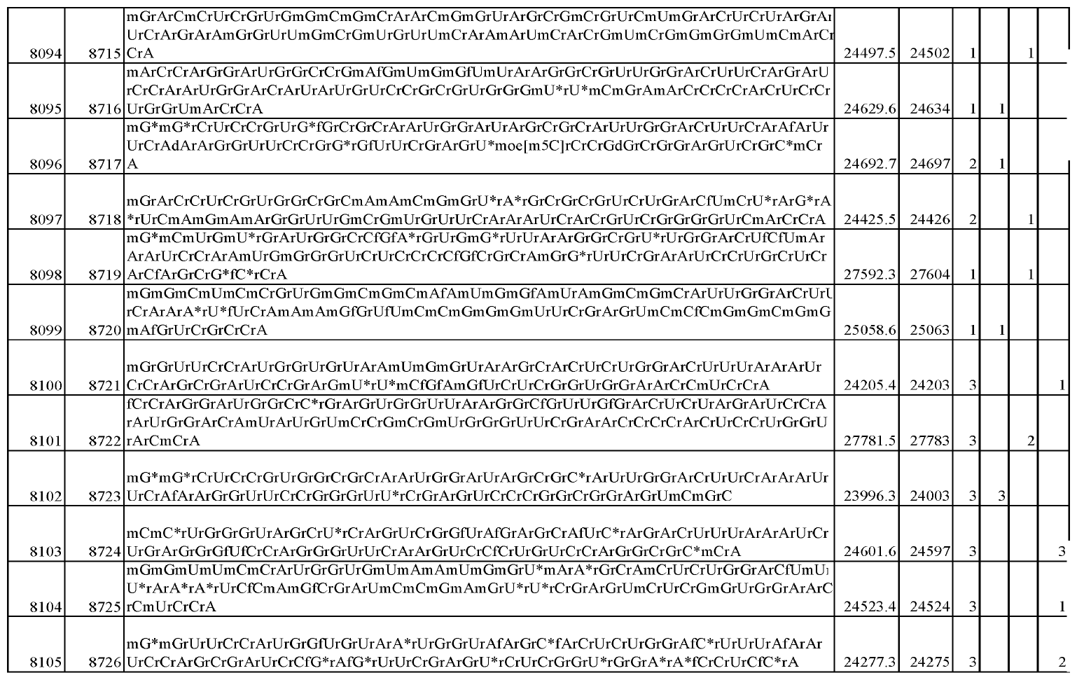

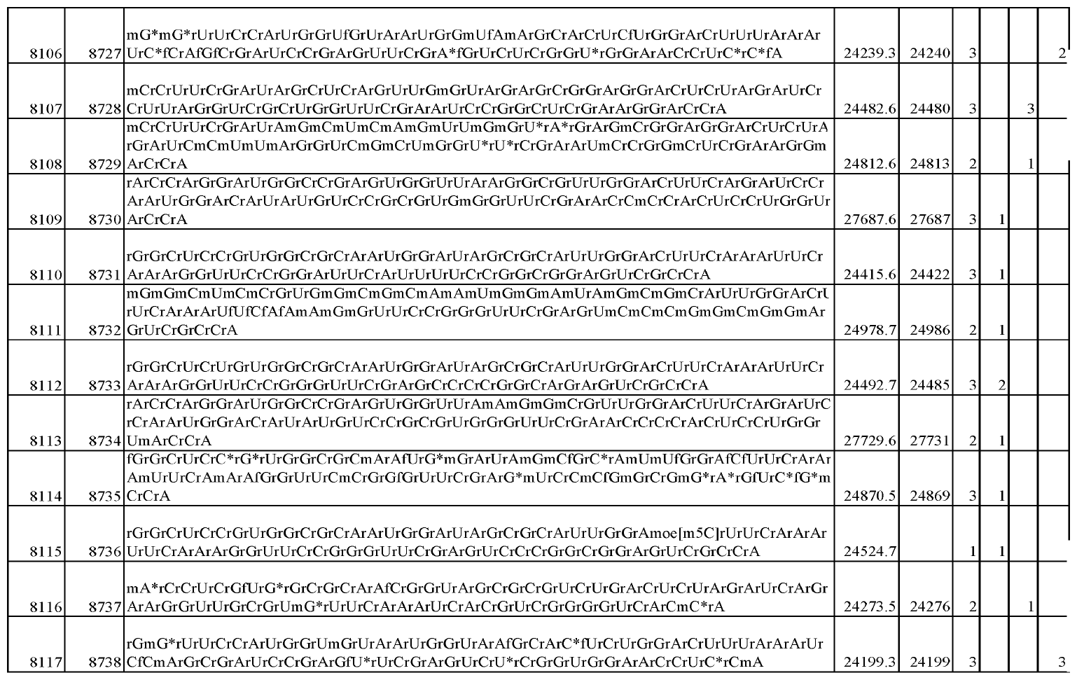

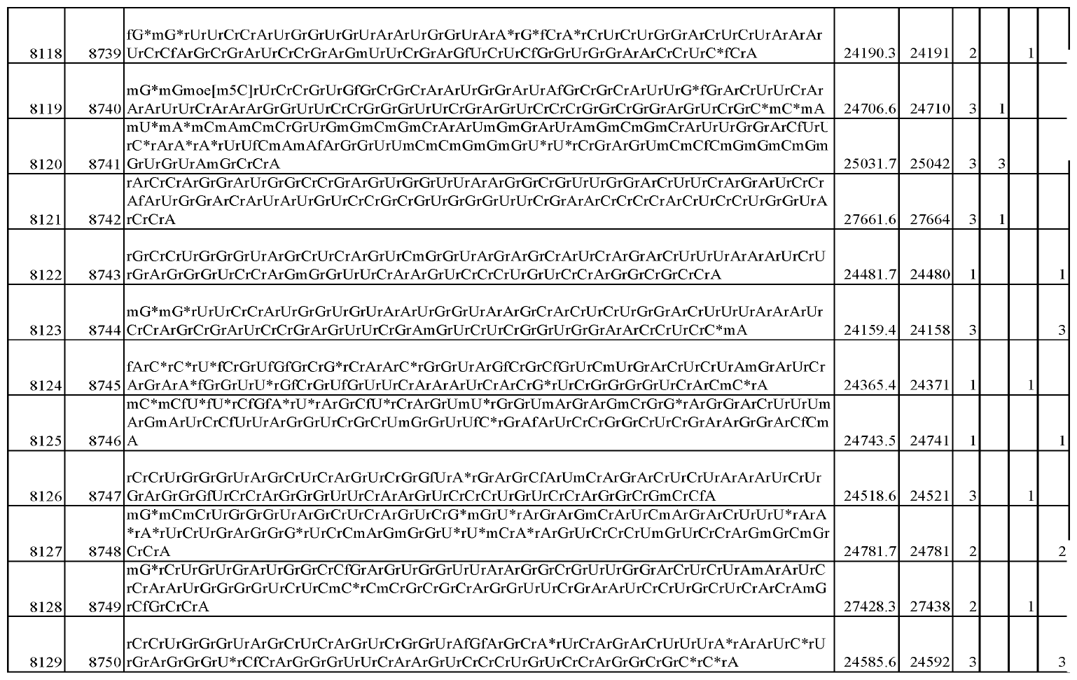

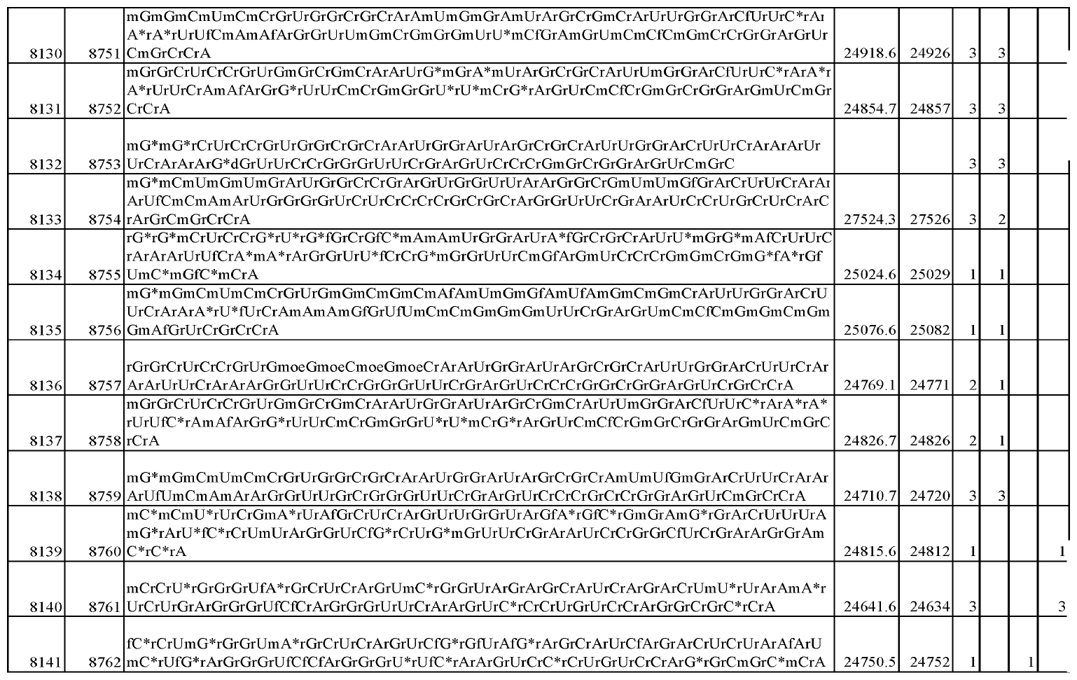

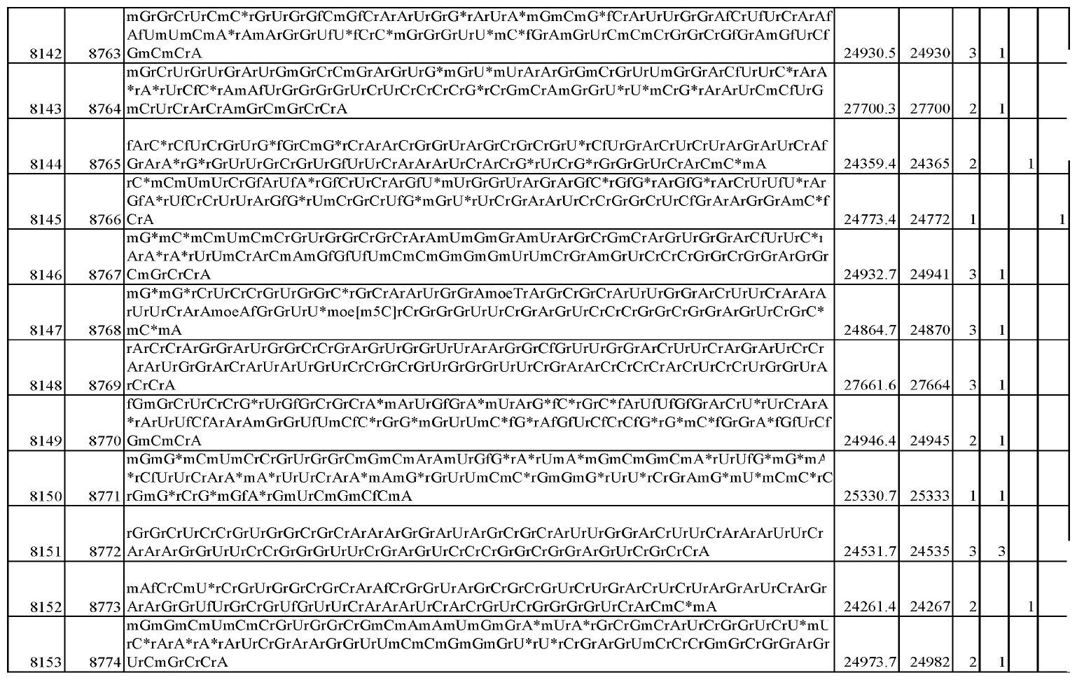

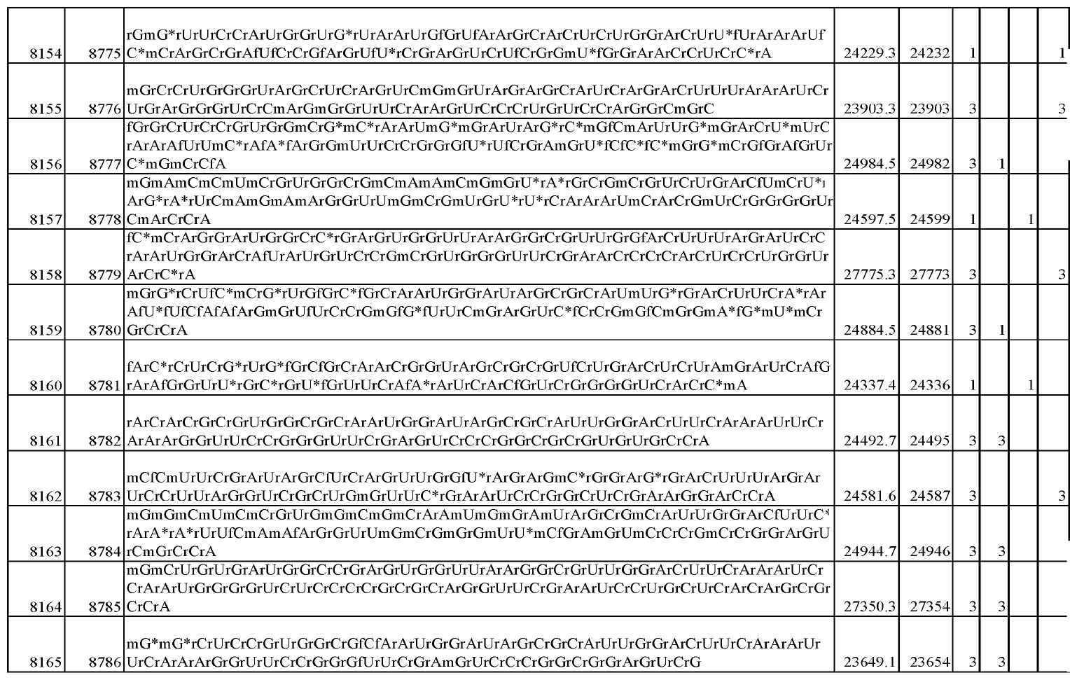

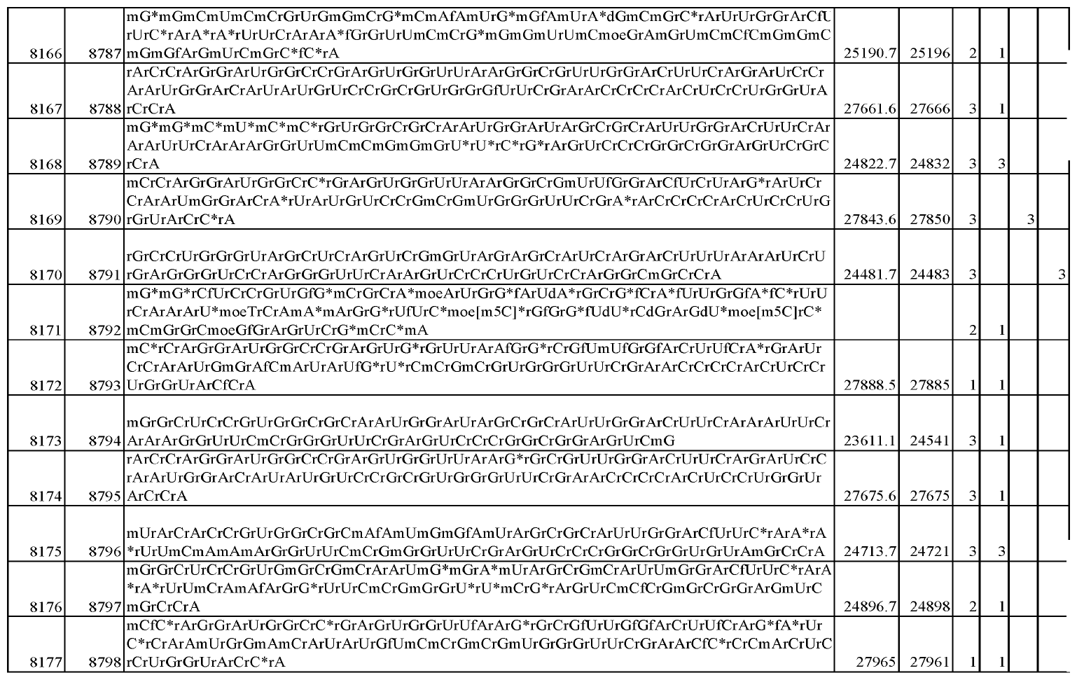

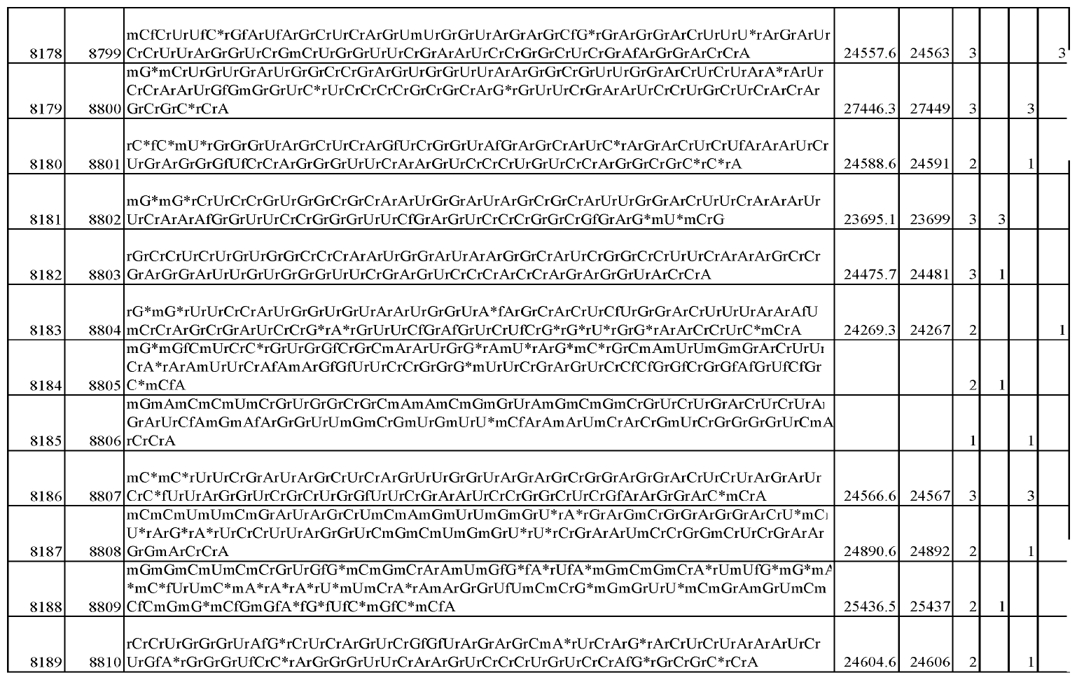

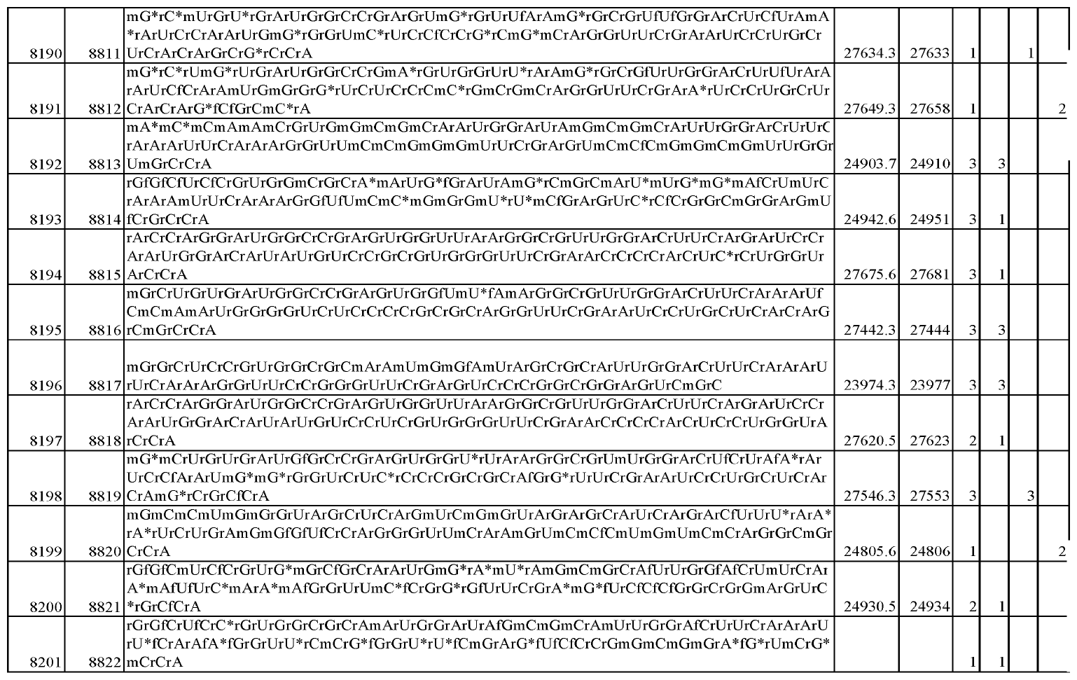

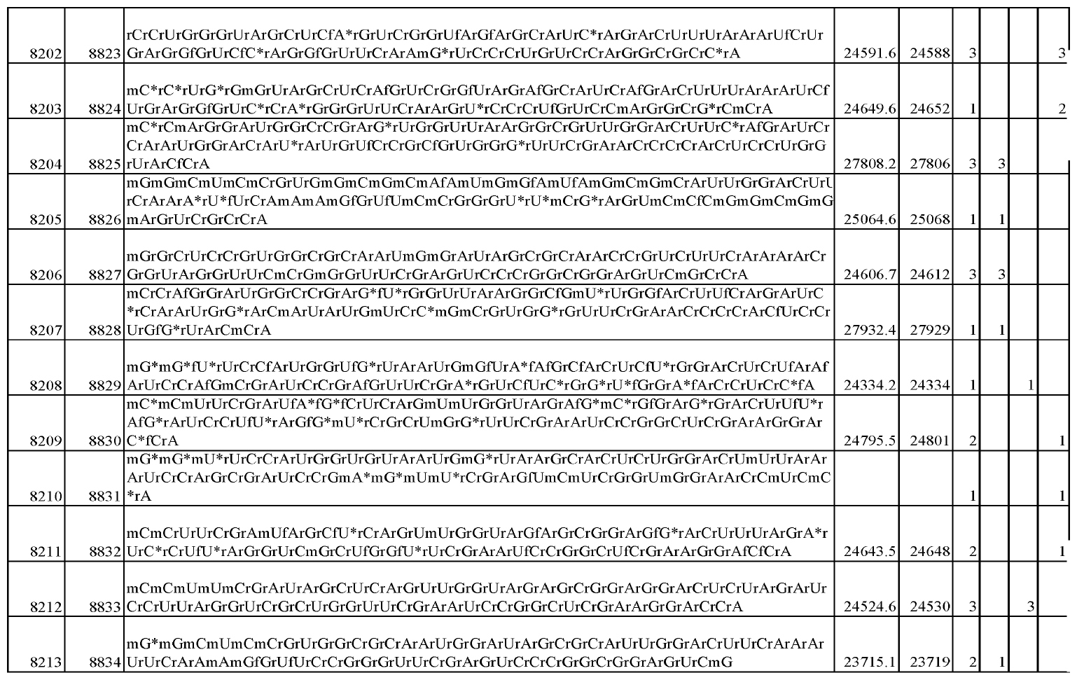

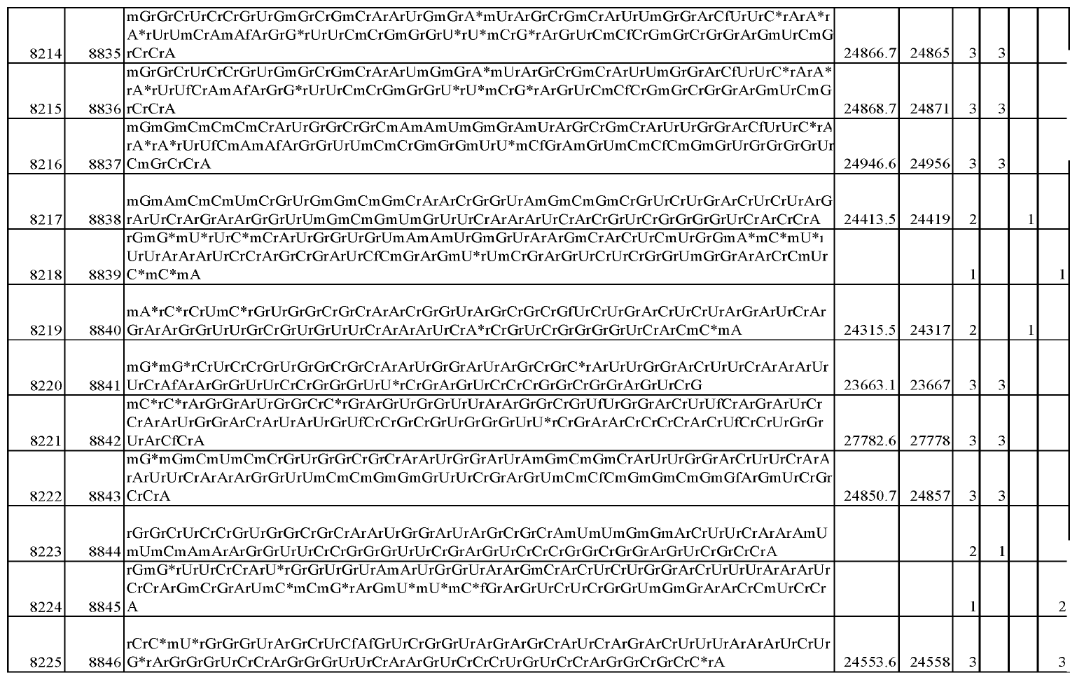

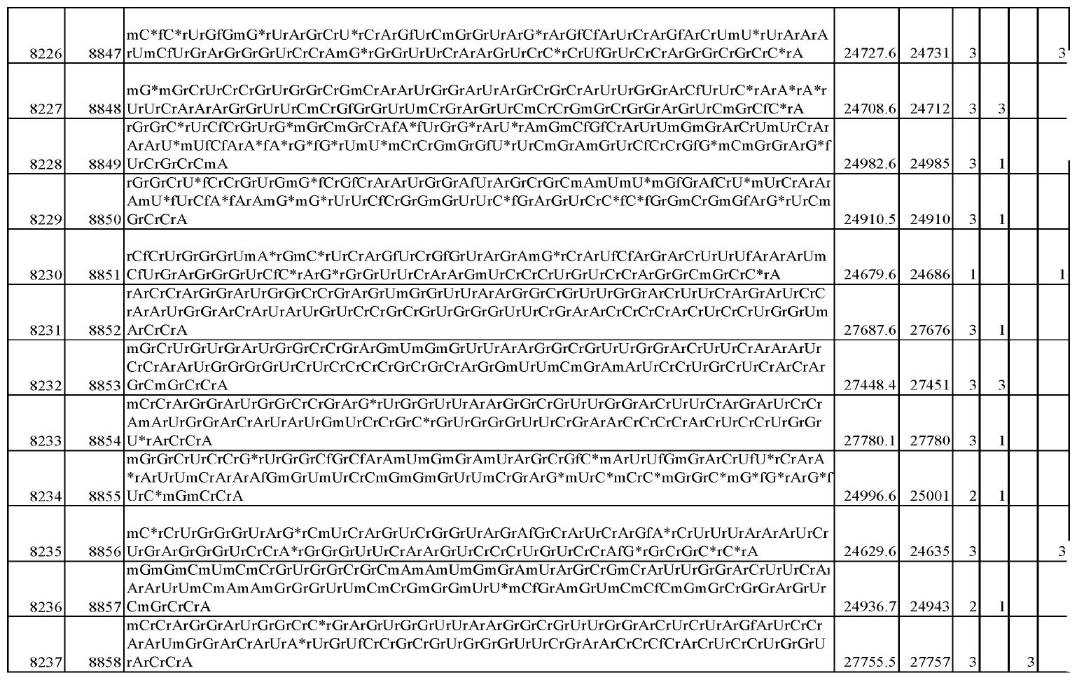

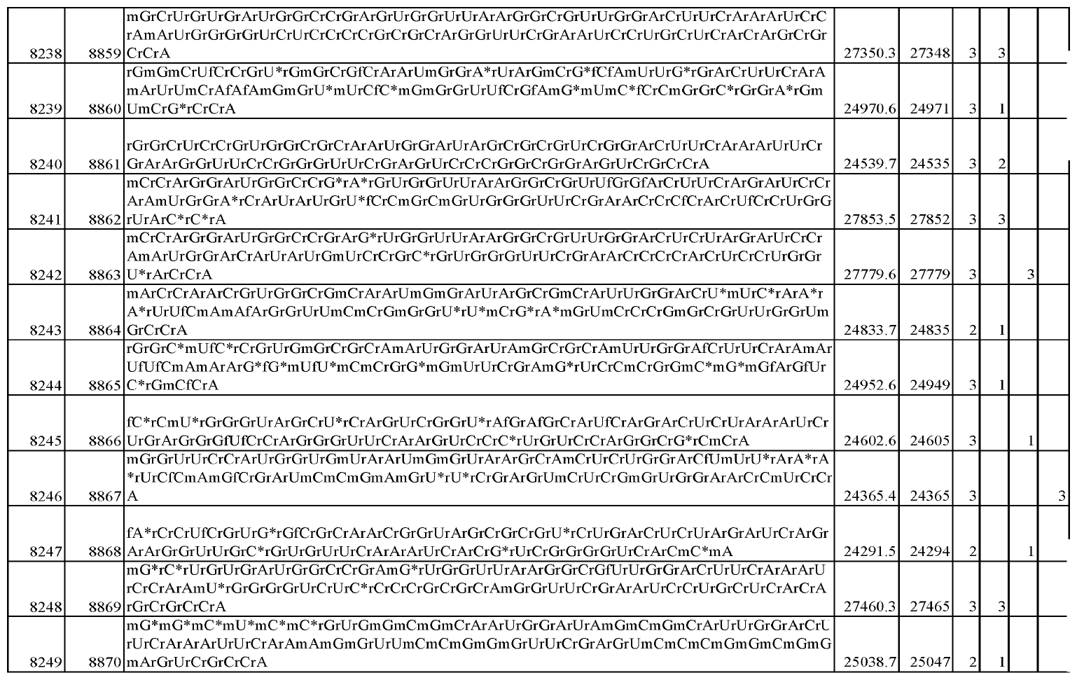

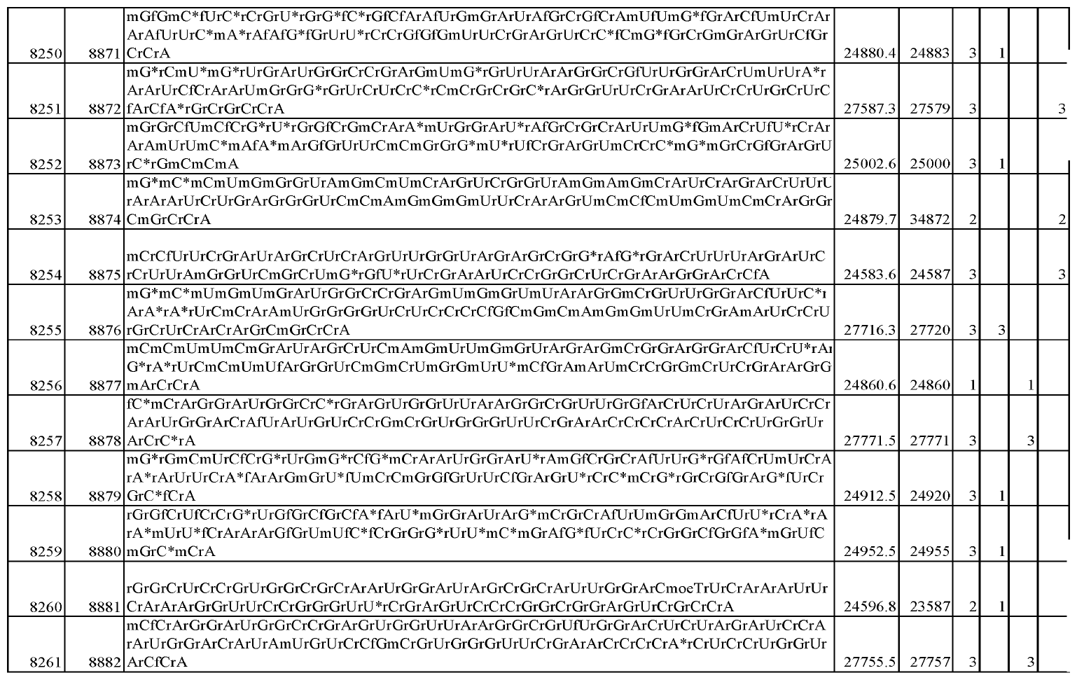

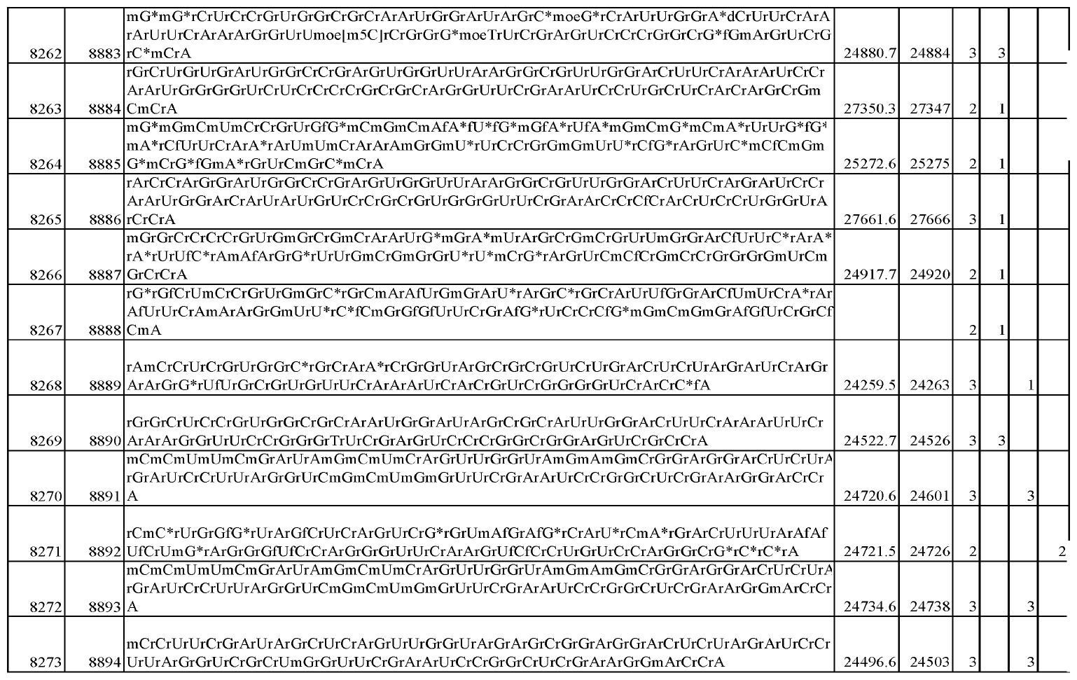

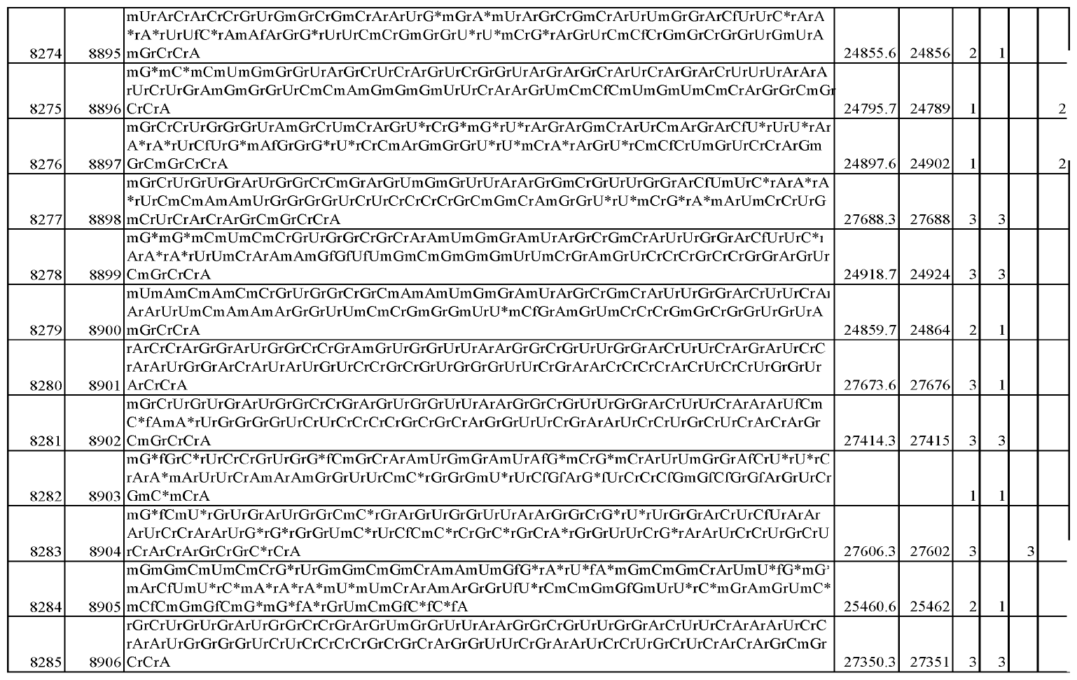

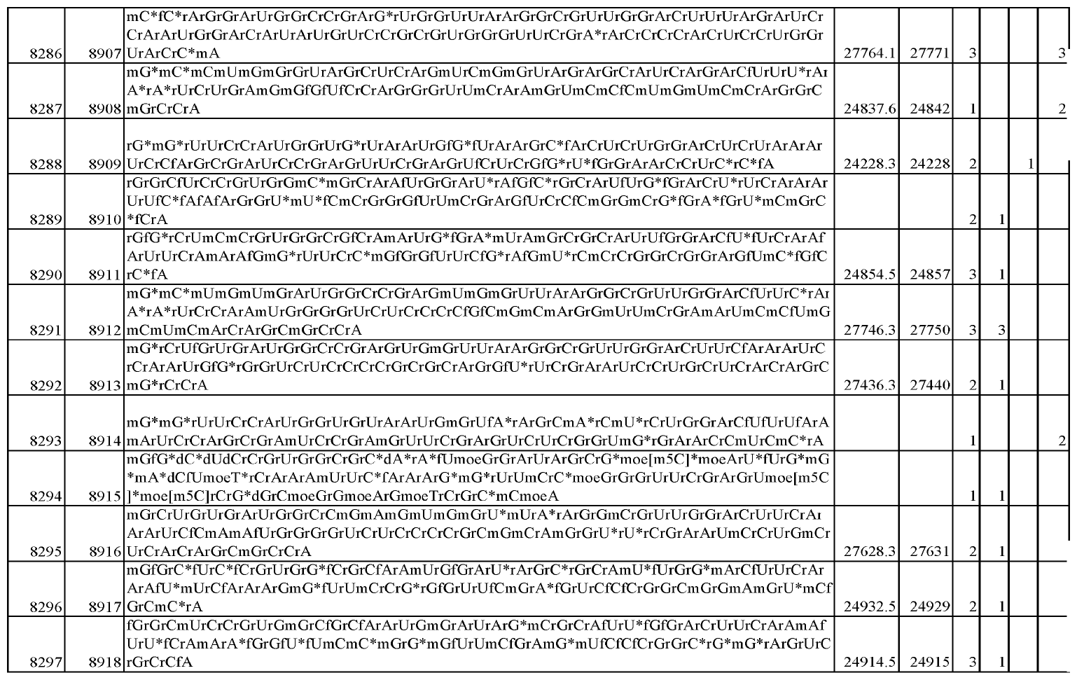

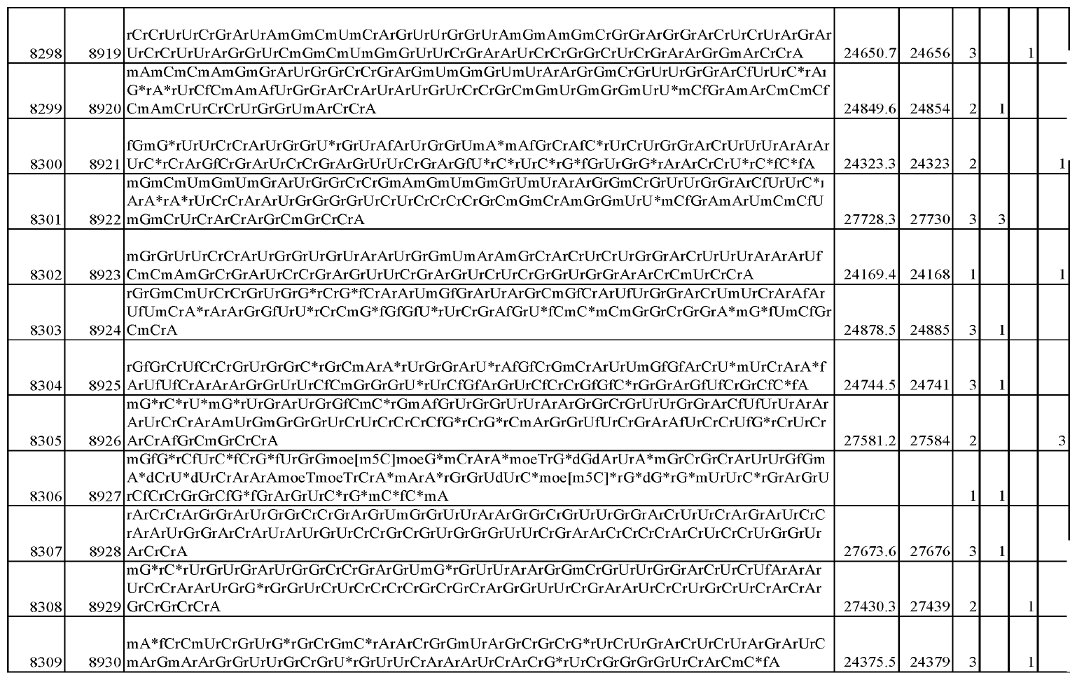

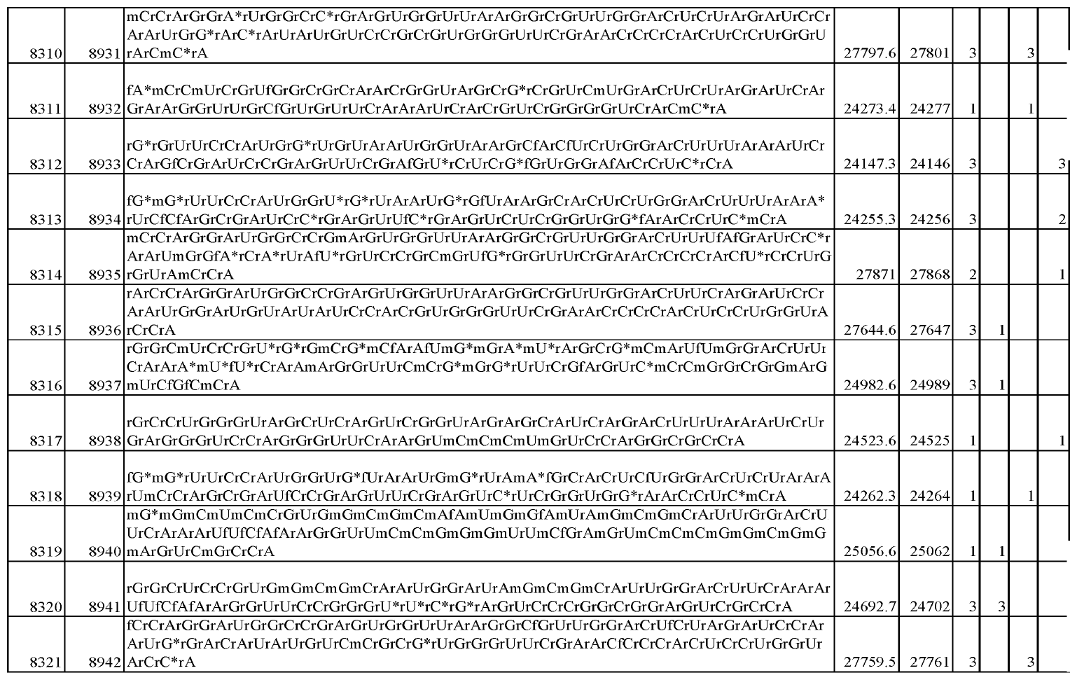

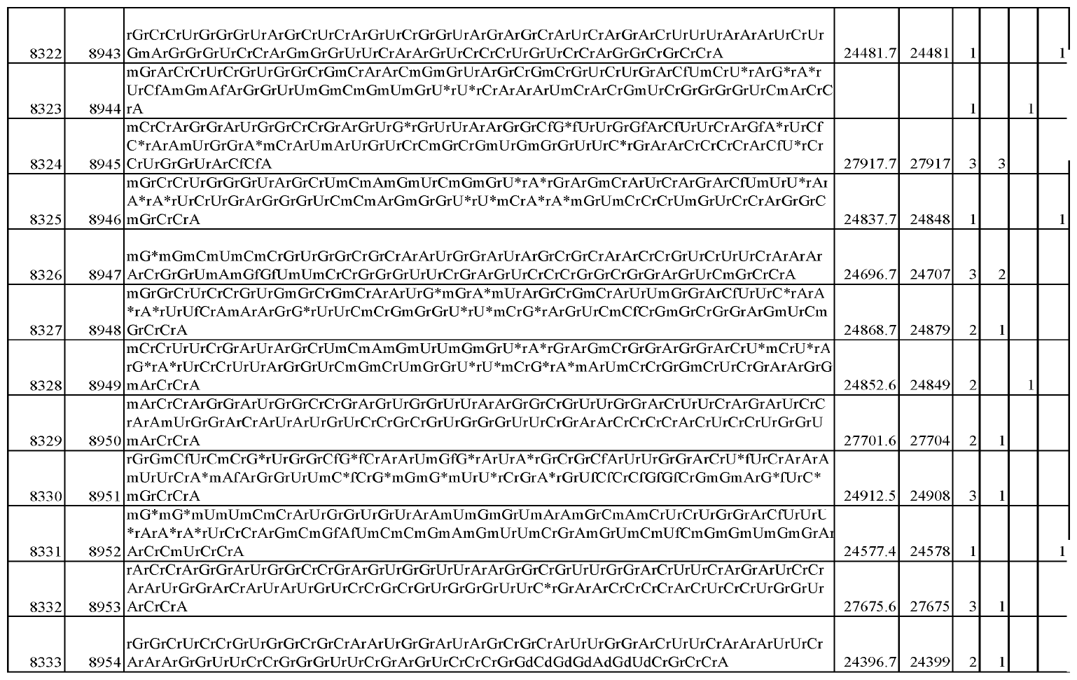

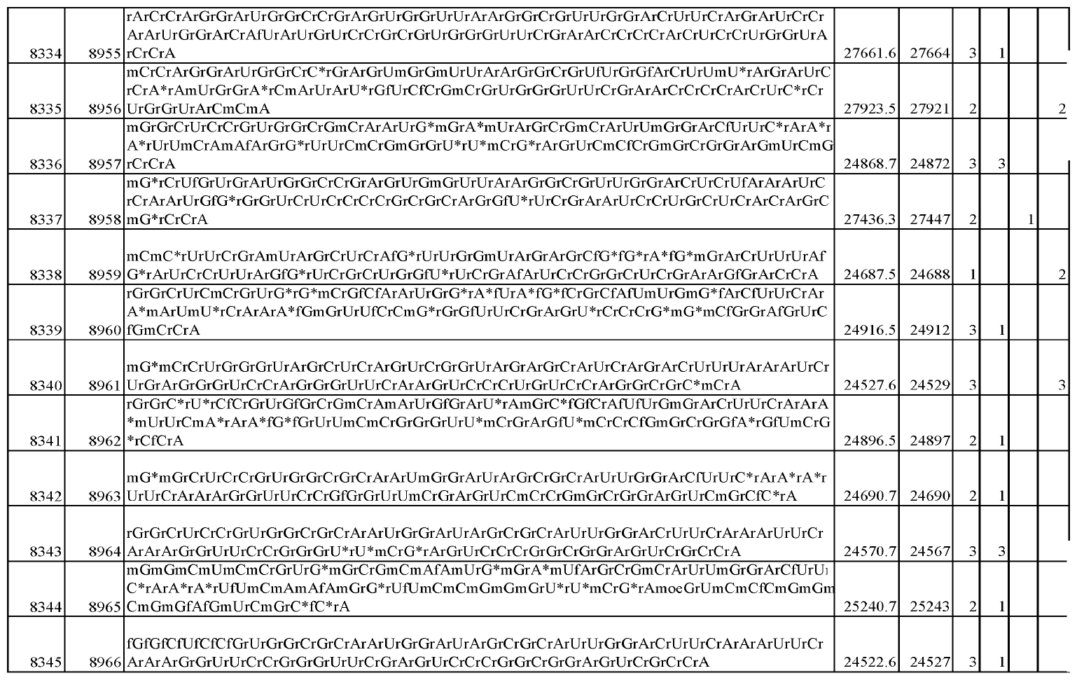

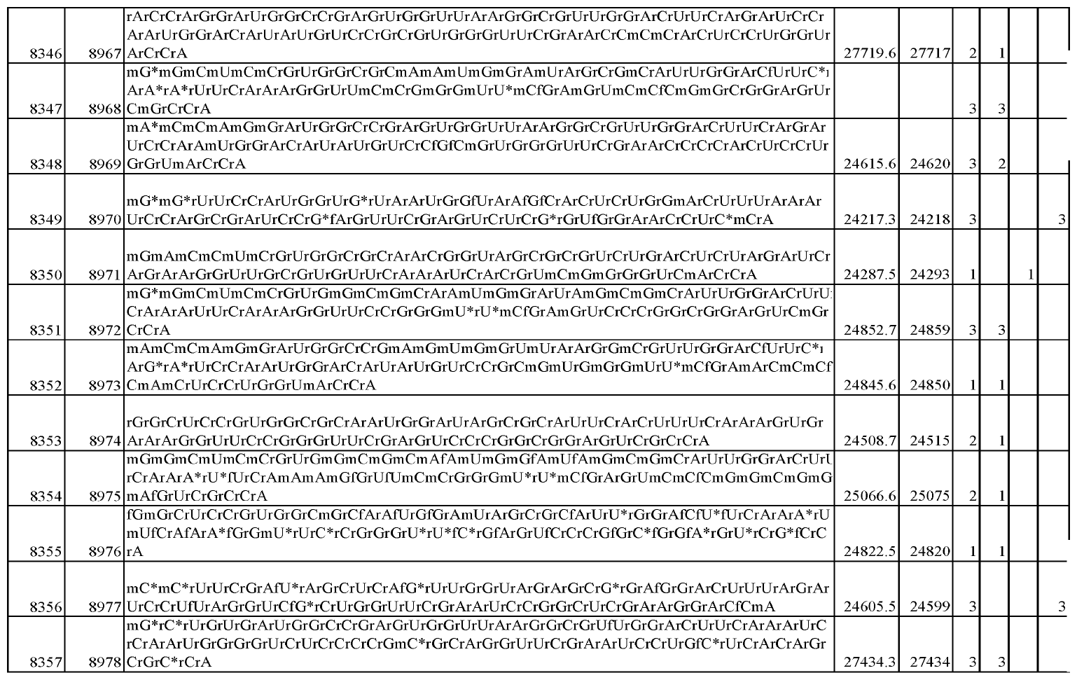

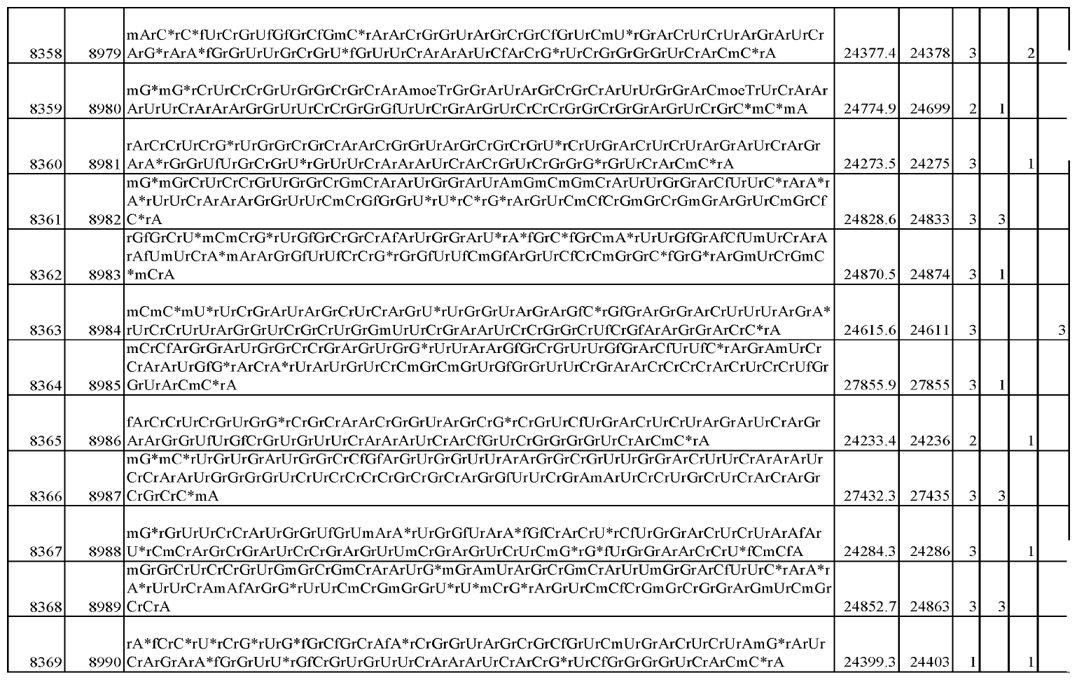

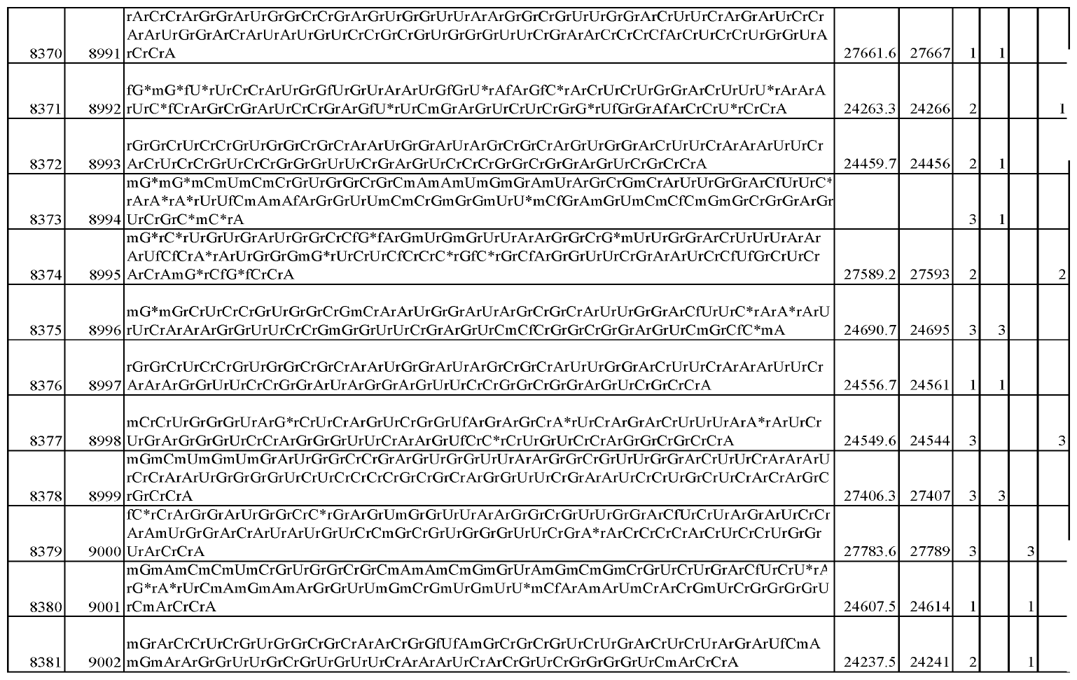

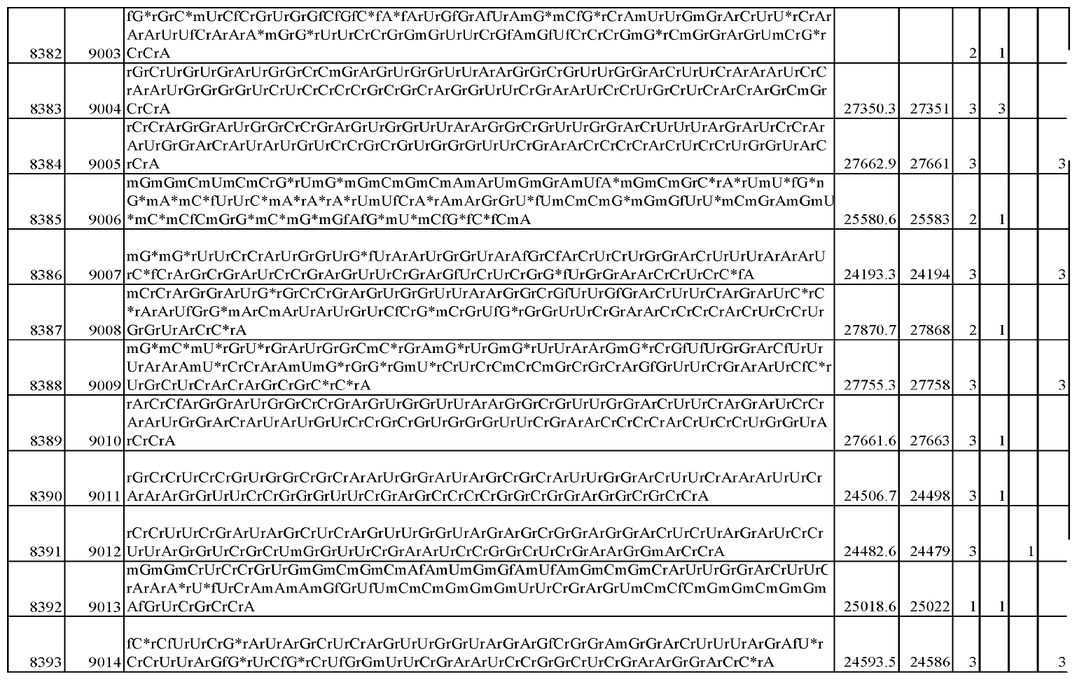

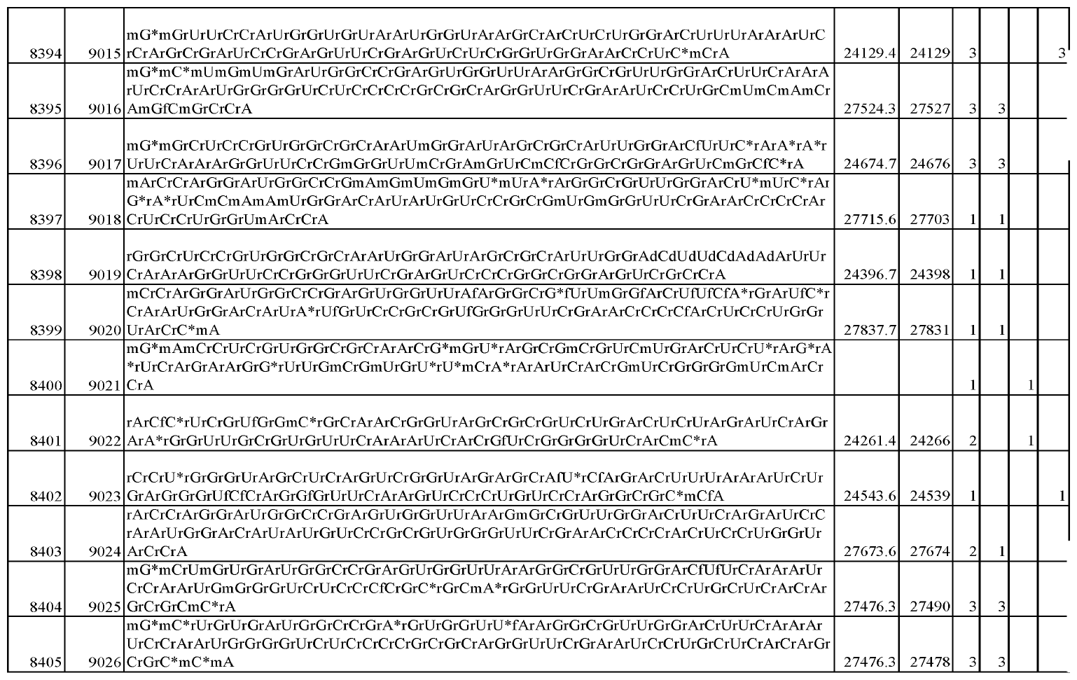

In some embodiments, LNPs are directed to specific tissues by the addition of targeting domains. For example, biological ligands may be displayed on the surface of LNPs to enhance interaction with cells displaying cognate receptors, thus driving association with and cargo delivery to tissues wherein cells express the receptor. In some embodiments, the biological ligand may be a ligand that drives delivery to the liver, e.g., LNPs that display GalNAc result in delivery of nucleic acid cargo to hepatocytes that display asialoglycoprotein receptor (ASGPR). The work of Akinc et al. Mol Ther 18 (7): 1357-1364 (2010) teaches the conjugation of a trivalent GalNAc ligand to a PEG-lipid (GalNAc-PEG-DSG) to yield LNPs dependent on ASGPR for observable LNP cargo effect (see, e.g., FIG. 6 of Akinc et al. 2010, supra). Other ligand-displaying LNP formulations, e.g., incorporating folate, transferrin, or antibodies, are discussed in WO2017223135, which is incorporated herein by reference in its entirety, in addition to the references used therein, namely Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; and Peer and Lieberman, Gene Ther. 2011 18:1127-1133.

In some embodiments, LNPs are selected for tissue-specific activity by the addition of a Selective ORgan Targeting (SORT) molecule to a formulation comprising traditional components, such as ionizable cationic lipids, amphipathic phospholipids, cholesterol and poly (cthylene glycol) (PEG) lipids. The teachings of Cheng et al. Nat Nanotechnol 15 (4): 313-320 (2020) demonstrate that the addition of a supplemental “SORT” component precisely alters the in vivo RNA delivery profile and mediates tissue-specific (e.g., lungs, liver, spleen) gene delivery and editing as a function of the percentage and biophysical property of the SORT molecule.

In some embodiments, the LNPs comprise biodegradable, ionizable lipids. In some embodiments, the LNPs comprise (9Z.12Z)-3-((4,4-bis (octyloxy) butanoyl)oxy)-2-((((3-(diethylamino) propoxy) carbonyl)oxy)methyl) propyl octadeca-9,12-dienoate, also called 3-((4,4-bis (octyloxy) butanoyl)oxy)-2-((((3-(diethylamino) propoxy) carbonyl)oxy)methyl) propyl (9Z,12Z)-octadeca-9,12-dienoate) or another ionizable lipid. Sec, e.g. lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, the average LNP diameter of the LNP formulation may be between 10s of nm and 100s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

A LNP may, in some instances, be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. In some embodiments, the zeta potential may describe the surface charge of an LNP. Lipid nanoparticles with relatively low charges, positive or negative, are generally desirable, as more highly charged species may interact undesirably with cells, tissues, and other elements in the body. In some embodiments, the zeta potential of a LNP may be from about-10 mV to about +20 mV, from about-10 mV to about +15 mV, from about-10 mV to about +10 mV, from about-10 mV to about +5 mV, from about-10 mV to about 0 mV, from about-10 mV to about-5 mV, from about-5 mV to about +20 mV, from about-5 mV to about +15 mV, from about-5 mV to about +10 mV, from about-5 mV to about +5 mV, from about-5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a TREM describes the amount of TREM that is encapsulated or otherwise associated with a LNP after preparation, relative to the initial amount provided. The encapsulation efficiency is desirably high (e.g., close to 100%). The encapsulation efficiency may be measured, for example, by comparing the amount of TREM in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free TREM in a solution. For the lipid nanoparticles described herein, the encapsulation efficiency of a TREM may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

A LNP may optionally comprise one or more coatings. In some embodiments, a LNP may be formulated in a capsule, film, or table having a coating. A capsule, film, or tablet including a composition described herein may have any useful size, tensile strength, hardness or density.

Additional exemplary lipids, formulations, methods, and characterization of LNPs are taught by WO2020061457, which is incorporated herein by reference in its entirety.

In some embodiments, in vitro or ex vivo cell lipofections are performed using Lipofectamine MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51 (34): 8529-8533 (2012), incorporated herein by reference in its entirety.

LNP formulations optimized for the delivery of CRISPR-Cas systems, e.g., Cas9-gRNA RNP, gRNA, Cas9 mRNA, are described in WO2019067992 and WO2019067910, both incorporated by reference.

Additional specific LNP formulations useful for delivery of nucleic acids are described in U.S. Pat. Nos. 8,158,601 and 8,168,775, both incorporated by reference, which include formulations used in patisiran, sold under the name ONPATTRO.

Exosomes can also be used as drug delivery vehicles for the TREM, TREM core fragment, TREM fragment, or TREM compositions or pharmaceutical TREM composition described herein. For a review, see Ha et al. July 2016. Acta Pharmaceutica Sinica B. Volume 6, Issue 4, Pages 287-296; https://doi.org/10.1016/j.apsb.2016.02.001.

Ex vivo differentiated red blood cells can also be used as a carrier for a TREM, TREM core fragment, TREM fragment, or TREM composition, or pharmaceutical TREM composition described herein. See, e.g., WO2015073587; WO2017123646; WO2017123644; WO2018102740; wO2016183482; WO2015153102; WO2018151829; WO2018009838; Shi et al. 2014. Proc Natl Acad Sci USA. 111 (28): 10131-10136; U.S. Pat. No. 9,644,180; Huang et al. 2017. Nature Communications 8:423; Shi et al. 2014. Proc Natl Acad Sci USA. 111 (28): 10131-10136.

Fusosome compositions, e.g., as described in WO2018208728, can also be used as carriers to deliver the TREM, TREM core fragment, TREM fragment, or TREM composition, or pharmaceutical TREM composition described herein.

Virosomes and virus-like particles (VLPs) can also be used as carriers to deliver a TREM, TREM core fragment, TREM fragment, or TREM composition, or pharmaceutical TREM composition described herein to targeted cells.

Plant nanovesicles, e.g., as described in WO2011097480A1, WO2013070324A1, or WO2017004526A1 can also be used as carriers to deliver the TREM, TREM core fragment, TREM fragment, or TREM composition, or pharmaceutical TREM composition described herein.

Delivery without a Carrier

A TREM, a TREM core fragment or a TREM fragment, a TREM composition or a pharmaceutical TREM composition described herein can be administered to a cell without a carrier, e.g., via naked delivery of the TREM, a TREM core fragment or a TREM fragment, a TREM composition or a pharmaceutical TREM composition.

In some embodiments, naked delivery as used herein refers to delivery without a carrier. In some embodiments, delivery without a carrier, e.g., naked delivery, comprises delivery with a moiety, e.g., a targeting peptide.

In some embodiments, a TREM, a TREM core fragment or a TREM fragment, or TREM composition, or pharmaceutical TREM composition described herein is delivered to a cell without a carrier, e.g., via naked delivery. In some embodiments, the delivery without a carrier, e.g., naked delivery, comprises delivery with a moiety, e.g., a targeting peptide.

### Use of TREMs

A TREM composition (e.g., a pharmaceutical TREM composition described herein) can modulate a function in a cell, tissue or subject. In embodiments, a TREM composition (e.g., a pharmaceutical TREM composition) described herein is contacted with a cell or tissue, or administered to a subject in need thereof, in an amount and for a time sufficient to modulate (increase or decrease) one or more of the following parameters: adaptor function (e.g., cognate or non-cognate adaptor function), e.g., the rate, efficiency, robustness, and/or specificity of initiation or elongation of a polypeptide chain; ribosome binding and/or occupancy; regulatory function (e.g., gene silencing or signaling); cell fate; mRNA stability; protein stability; protein transduction; protein compartmentalization. A parameter may be modulated, e.g., by at least 5% (e.g., at least 10%, 15%, 20%, 25%, 30%, 40%, 50%, 60%, 70%, 80%, 90%, 100%, 150%, 200% or more) compared to a reference tissue, cell or subject (e.g., a healthy, wild-type or control cell, tissue or subject).

All references and publications cited herein are hereby incorporated by reference.

The following examples are provided to further illustrate some embodiments of the present invention, but are not intended to limit the scope of the invention; it will be understood by their exemplary nature that other procedures, methodologies, or techniques known to those skilled in the art may alternatively be used.

### EXAMPLES

TABLE of
Contents for Examples

| | |
|---|---|
| Example 1 | Synthesis of modified TREMs |
| Example 2 | HPLC and MS analysis of modified TREMs |
| Example 3 | Analysis of modified TREMs via anion-exchange HPLC |
| Example 4 | Analysis of TREMs via PAGE Purification and Analysis |
| Example 5 | Deprotection of synthesized TREM |
| Example 6 | Characterization of chemically modified TREMs for |
| | readthrough of a premature termination codon (PTC) in a |
| | reporter protein |
| Example 7 | Characterization of chemically modified TREMs for |
| | readthrough of a premature termination codon (PTC) in a |
| | disease reporter cell line |
| Example 8 | Correction of a missense mutation in an ORF with |
| | administration of a TREM |
| Example 9 | Evaluation of protein expression levels of SMC-containing |
| | ORF with administration of a TREM |
| Example 10 | Modulation of translation rate of SMC-containing ORF |
| | with TREM administration |
| Example 11 | Rescue of full-length GLA expression and activity in Fabry |
| | patient fibroblasts upon administration of an exemplary |
| | TREM |
| Example 12 | In Vivo PTC Readthrough and Target Engagement of |
| | TREM by Hydrodynamic Gene Delivery |
| Example 13 | In Vivo Administration of a Lipid Nanoparticle TREM |
| | Formulation |

### Example 1: Synthesis of Modified TREMs

Generally, TREM molecules (e.g., modified TREMs) may be chemically synthesized and purified by HPLC according to standard solid phase synthesis methods using phosphoramidite chemistry. (see, e.g., Scaringe S. et al. (2004) Curr Protoc Nucleic Acid Chem, 2.10.1-2.10.16; Usman N. et al. (1987) J. Am. Chem. Soc, 109, 7845-7854). Individually modified TREM molecules containing one or more 2′-methoxy (2′OMe), 2′fluoro (2′F), 2′-methoxyethyl (2′-MOE), or phosphorothioate (PS) modifications were prepared using either TREM-Arg-TGA, TREM-Ser-TAG, or TREM-Gln-TAA sequences as a framework according to phosphoramidite technology on solid phase used in oligonucleotide synthesis. For clarity, the arginine non-cognate TREM molecule named TREM-Arg-TGA contains the sequence of ARG-UCU-TREM body but with the anticodon sequence corresponding to UCA instead of UCU (i.e., SEQ ID NO: 622). Simlarly, a serine non-cognate TREM molecule named TREM-Ser-TAG contains the sequence of SER-GCU-TREM body but with the anticodon sequence corresponding to CUA instead of GCU (i.e., SEQ ID NO: 623). A glutamine non-cognate TREM molecule named TREM-Gln-TAA contains the sequence of GLN-CUG-TREM body but with the anticodon sequence corresponding to UUA instead of CUG (i.e., SEQ ID NO: 624).

To make the 2′OMe modified TREMs, the following 2′-O-methyl phosphoramidites were used: (5′-O-dimethoxytrityl-N6-(benzoyl)-2′-O-methyl-adenosine-3′-O-(2-cyanocthyl-N,N-diisopropy-lamino) phosphoramidite, 5′-O-dimethoxy-trityl-N4-(acetyl)-2′-O-methyl-cytidine-3′-O-(2-cyanocthyl-N,N-diisopropyl-amino)phosphoramidite, (5′-O-dimethoxytrityl-N2-(isobutyryl)-2′-O-methyl-guanosine-3′-O-(2-cyano-ethyl-N,N-diisopropylamino)-phosphoramidite, and 5′-O-dimethoxy-trityl-2′-O-methyluridine-3′-O-(2-cyanoethyl-N,N-diisopropylamino) phosphoramidite. To make the 2′-deoxy and 2′-F modified TREMs, analogous 2′-deoxy and 2′-fluoro-phosphoramidites with the same protecting groups as the 2′-O-methyl RNA amidites were used. To make the 2′-MOE modified TREMs, the following 2′-MOE-phosphoramidites were used: 5′-O-(4,4′-Dimethoxytrityl)-2′-O-methoxyethyl-N6-benzoyl-adenosine-3′-O-[(2-cyanocthyl)-(N,N-diisopropyl)]-phosphoramidite, 5′-O-(4,4′-Dimethoxytrityl)-2′-O-methoxyethyl-5-methyl-N4-benzoyl-cytidine-3′-O-[(2-cyanocthyl)-(N,N-diisopropyl)]-phosphoramidite, 5′-O-(4,4′-Dimethoxytrityl)-2′-O-methoxyethyl-N2-isobutyryl-guanosine-3′-O-[(2-cyanocthyl)-(N,N-diisopropyl)]-phosphoramidite, 5′-O-(4,4′-Dimethoxytrityl)-2′-O-methoxyethyl-5-methyl-uridine-3′-O-[(2-cyanoethyl)-(N,N-diisopropyl)]-phosphoramidite.

During the oligonucleotide synthesis via this phosphoramidites approach, the phosphorothioate was introduced by oxidizing the phosphite triester using a sulfur transfer reagent, such as tetraethylthiuram disulfide (TETD), bis (O,O-diisopropoxy phosphinothioyl) disulfide (Stec's reagent), 3H-1,2-benzodithiol-3-one-1,1,-dioxide (Beaucage reagent), phenylacetyl disulfide (PADS), 3-ethoxy-1,2,4-dithiazoline-5-one (EDITH), 1,2-dithiazole-5-thione (xanthane hydride or ADTT), 3-((dimethylamino-methylidene)amino)-3H-1,2,4-dithiazole-3-thione (DDTT), dimethylthiuram disulfide (DTD), 3-phenyl-1,2,4-dithiazoline-5-one (PolyOrg Sulfa or POS).

FIG. 2 describes a series of singly and multiply modified TREMs synthesized according to this procedure. The sequences of each of these TREMs are provided in the table, wherein r: ribonucleotide; m: 2′-OMe; *: PS linkage; f: 2′-fluoro; moe: 2′-moc; d: deoxyribonucleotide; 5MeC: 5-methylcytosine. Thus, for example, mA represents 2′-O-methyl adenosine, moe5MeC represents 2′-MOE nucleotide with 5-methylcytosine nucleobase, and dA represents an adenosine deoxyribonucleotide.

### Example 2: HPLC and MS Analysis of Modified TREMs

Chemically modified TREM molecules may be analyzed by HPLC, for example, to evaluate the purity and homogeneity of the compositions. A Waters Aquity UPLC system using a Waters BEH C18 column (2.1 mm×50 mm×1.7 μm) may be used for this analysis. Samples may be prepared by dissolving 0.5 nmol of the TREM in 75 μL of water and injecting 2 μL of the solution. The buffers used may be 50 mM dimethylhexylammonium acetate with 10% CH₃CN (acetonitrile) as buffer A and 50 mM dimethylhexylammonium acetate with 75% CH₃CN as buffer B (gradient 25-75% buffer B over 5 mins), with a flow rate of 0.5 mL/min at 60° C. ESI-LCMS data for the chemically modified TREMs may be acquired on a Thermo Ultimate 3000-LTQ-XL mass spectrometer.

FIG. 2 describe a series of singly and multiply modified TREMs synthesized according to the protocol outlined in Example 1. The calculated and detected molecular weights for each sequence were determined as outlined herein.

### Example 3: Analysis of modified TREMs via anion-exchange HPLC

This example describes the quality control of a synthesized TREM via anion-exchange HPLC. Using the Dionex DNA-Pac-PA-100 column, a gradient is employed using HPLC buffer A and HPLC buffer B. 0.5 ODUs of a sample that has been dissolved in H20 or Tris buffer, pH 7.5 is injected onto the gradient. The gradient employed is based on oligonucleotide length and can be applied according to Table 13. The parameters provided in Table 14 can be used to program a linear gradient on the HPLC analyzer.

TABLE 13
Oligonucleotide length and gradient percentages

| Length | Gradient |
|---|---|
| (bases) | (% B) |
| 0-5 | 0-30 |
| 6-10 | 10-40 |
| 11-16 | 20-50 |
| 17-32 | 30-60 |
| 33-50 | 40-70 |
| >50 | 50-80 |

TABLE 14
Parameters for a linear gradient on HPLC analyzer

| Time | Flow | % Buffer | % Buffer |
|---|---|---|---|
| (min) | (mL/min) | A | B |
| 0 | 1.5 | 100 | 0 |
| 1 | 1.5 | 100 | 0 |
| 3 | 1.5 | 70a | 30a |
| 15 | 1.5 | 40a | 60a |
| 15.5 | 2.5 | 0 | 100 |
| 17 | 2.5 | 0 | 100 |
| 17.25 | 2.5 | 100 | 0 |
| 23 | 2.5 | 100 | 0 |
| s23.1 | 1.5 | 100 | 0 |
| 24 | 1.5 | 100 | 0 |
| 25 | 0.1 | 100 | 0 |

### Example 4: Analysis of TREMs via PAGE Purification and Analysis

This example describes the quality control of a synthesized TREM via PAGE Purification and Analysis. Gel purification and analysis of 2′-ACE protected RNA follows standard protocols for denaturing PAGE (Ellington and Pollard (1998) In Current Protocols in Molecular Biology, Chanda, V). Briefly, the 2′-ACE protected oligo is resuspended in 200 mL of gel loading buffer. Invitrogen™ NuPAGE™ 4-12% Bis-Tris Gels or similar gel is prepared in gel apparatus. Samples are loaded and gel ran at 50-120 W, maintaining the apparatus at 40° C. When complete, the gel is exposed to ultraviolet (UV) light at 254 nm to visualize the purity of the RNA using UV shadowing. If necessary, the desired gel band is excised with a clean razor blade. The gel slice is crushed and 0.3M NaOAc elution buffer is added to the gel particles, and soaked overnight. The mixture is decanted and filtered through a Sephadex column such as Nap-10 or Nap-25.

### Example 5: Deprotection of Synthesized TREM

This example describes the deprotection of a TREM made according to an in vitro synthesis method. The 2′-protecting groups are removed using 100 mM acetic acid, pH 3.8. The formic acid and ethylene glycol byproducts are removed by incubating at 60° C. for 30 min followed by lyophilization or SpeedVac-ing to dryness. After this final deprotection step, the oligonucleotides are ready for use.

### Example 6. Characterization of Chemically Modified TREMs for Readthrough of a Premature Termination Codon (PTC) in a Reporter Protein

This example describes an assay to test the ability of a non-cognate chemically modified TREM to readthrough a PTC in a cell line expressing a reporter protein having a PTC. This protocol describes analysis of chemically modified arginine, serine, and glutamine non-cognate TREM (i.e., Arg-TGA, Ser-TAG, Gln-TAA, Lys-TAA, Leu-TGA, Trp-TAG, Gln-TAG, Ser-TAA, Ser-TGA, Tyr-TAA, Tyr-TAG), though a non-cognate TREM specifying any one of the other amino acids can also be used.

A cell line engineered to stably express the NanoLuc reporter construct containing a premature termination codon (PTC) may be generated using the FlpIn and/or JumpIN cell system (ThermoFisher Scientific, USA) according to the manufacturer's instructions. The NanoLuc reporter can also be modified with a destabilizing PEST domain at its C-terminus to reduce the protein half-life. Delivery of the chemically modified TREMs into the NanoLuc reporter cells is carried out via a reverse transfection reaction using lipofectamine RNAiMAX (ThermoFisher Scientific, USA) according to manufacturer instructions. Briefly, 5 μL of a 2.5 uM solution of chemically modified TREM sample are diluted in a 20 uL RNAiMAX/OptiMEM mixture. After 30 min gentle mixing at room temperature, the 25 uL TREM/transfection mixture is added to a 96-well plate and kept still for 20-30 min before adding the cells stably expressing the NanoLuc reporter construct containing a PTC. The NanoLuc reporter cells are harvested and diluted to 4x 10⁵ cells/mL in complete growth medium, and 100 μL of the diluted cell suspension is added and mixed to the plate containing the TREM.

To monitor the efficacy of the chemically modified TREM to read through the PTC in the reporter construct at 24 or 48 hours after TREM delivery into cells, a NanoGlo bioluminescent assay (Promega, USA) may be performed according to manufacturer instruction. Briefly, for cells that are to be assayed after 48 hours, 100 uL complete growth medium is added to the 96-well plate 24 hours post-transfection to dilute the transfection reagent for cell health. At the time of harvest, either 24- or 48-hours post-transfection, cell media is replaced and allowed to equilibrate to room temperature. NanoGlo reagent is prepared by mixing the buffer with substrate in a 50:1 ratio. 50 μL of mixed NanoGlo reagent is added to the 96-well plate and mixed on the shaker at 600 rpm for 10 min. Then the plate is centrifuged at 1000g for 2 min, followed by a 5 min incubation step at room temperature before measuring sample bioluminescence. As a positive control, a host cell expressing the NanoLuc reporter construct without a PTC is used. As a negative control, a host cell expressing the NanoLuc reporter construct with a PTC is used, but no TREM is transfected. The efficacyof the chemically modified TREMs is measured as a ratio of the NanoLuc luminescence in the experimental sample to the NanoLuc luminescence of the positive control or as a ratio of the NanoLuc luminescence in the experimental sample to the NanoLuc luminescence of the negative control. It is expected that if the sample TREM is functional, it may be able to read-through the stop mutation in the NanoLuc reporter and produce a luminescent reading higher than the luminescent reading measured in the negative control. If the sample TREM is not functional, the stop mutation is not rescued, and luminescence less or equal to the negative control is detected.

The impacts of chemical modifications were evaluated in singly and multiply modified TREM sequences and are summarized in FIG. 2. In this figure, the TREMs are annotated as follows: r: ribonucleotide; m: 2′-OMe; *: PS linkage; f: 2′-fluoro; moc: 2′-moc; d: deoxyribonucleotide; 5MeC: 5-methylcytosine. Thus, for example, mA represents 2′-O-methyl adenosine, moc5MeC represents 2′-MOE nucleotide with 5-methylcytosine nucleobase, and dA represents an adenosine deoxyribonucleotide.

FIG. 2 also summarizes the results of the activity screen in column “A” for measurements made using NanoLuc reporter cells at 48 hours post-transfection, which reported as log 2 fold changes compared with the appropriate unmodified TREM, wherein “1” indicates less than a 1 log 2 fold change; “2” indicates greater than or equal to 1 and less than 3.32 log 2 fold change; and “3” indicates greater than or equal to 3.32 log 2 fold change. The results show that certain modifications were tolerated at many positions, but particular sites were sensitive to modification or exhibited improved activity when modified

### Example 7: Characterization of Chemically Modified TREMs for Readthrough of a Premature Termination Codon (PTC) in a Disease Reporter Cell Line

This example describes an assay to test the ability of an exemplary TREM to readthrough a PTC in a cell line expressing a disease reporter protein bearing the PTC.

### Host Cell Modification

A cell line engineered to stably express a HaloTag and HiBiT-tagged disease reporter construct containing a premature termination codon (PTC), such as Factor IX at position 298 (FIXR298X), Factor IX at position 29 (FIXR29X), Factor IX at position 44 (FIXQ++X), Tripeptidyl-peptidase 1 at position 208 (TPP1^{R208X}) , Protocadherin Related 15 at position 245 (PCDH15^{R245X}), or Rhodopsin at position 334 (Rho^{S334X}) was generated using the Jump-In system according to manufacturer's instructions. Briefly, Jump-In GripTite HEK293 (Thermo Scientific A14150) cells were co-transfected with an expression vector containing the disease reporter, such as pJTI-R⁴-DEST-CMV—FIX-R298X-HaloTag-HiBiT-pA for FIX^{R298X} to make the Factor IX disease reporter expressing cell line, and a pJTI-R4-Int PhiC31 integrase expression vector using Lipofectamine2000 according to manufacturer's instructions. After 24 hours, the media was replaced with fresh media. The next day, the cells were re-seeded at 50% confluency and selected with 10 ug/mL Blasticidin and 600 ug/mL G418 for 7 days with media change every 2 days. The remaining cells were expanded and tested for reporter construct expression.

### Translation Suppression Assay

Exemplary TREMs were synthesized and characterized as described herein, then transfected into cells. Forty-eight hours after TREM delivery into cells, conditioned media was collected, fresh media was added to the cells and allowed to equilibrate to room temperature. To measure the efficacy of arginine TREMs in PTC readthrough, full-length HiBiT-tagged disease reporter protein was assayed in both cells, and 48-hour conditioned media. Briefly, reconstituted Nano-Glo® HiBiT Lytic Reagent was added to both cells containing fresh media and 48-hour conditioned media at a 1:1 v/v ratio, mixed on an orbital shaker at 500 rpm for 10 minutes, and incubated at room temperature for 10 minutes. The HiBiT-tagged disease reporter activity is measured by reading the luminescence in a plate reader. The results of this experiment in the three HiBiT-tagged disease reporter constructs is shown below in Table 21. In this table, the results for each TREM tested are reported as log 2 fold changes compared with the appropriate unmodified TREM (TREM NO. 2309), wherein “1” indicates less than a −0.05 log 2 fold change; “2” indicates greater than or equal to −0.05 and less than 0.55 log 2 fold change; and “3” indicates greater than or equal to 0.55 log 2 fold change.

TABLE 21

| | | TPP1 | PCDH15 | FIX |
|---|---|---|---|---|
| TREM | SEQ ID | (R208X) | (R245X) | (R298X) |
| NO. | NO. | 100 nM | 100 nM | 100 nM |
| 4 | 625 | 3 | 3 | 3 |
| 7 | 628 | 3 | 3 | 3 |
| 24 | 645 | 3 | 3 | 3 |
| 29 | 650 | 3 | 3 | 3 |
| 47 | 668 | 3 | 3 | 3 |
| 61 | 682 | 3 | 3 | 3 |
| 77 | 698 | 3 | 3 | 3 |
| 78 | 699 | 3 | 3 | 3 |
| 84 | 705 | 3 | 3 | 3 |
| 96 | 717 | 3 | 3 | 3 |
| 103 | 724 | 3 | 3 | 3 |
| 124 | 745 | 3 | 3 | 3 |
| 125 | 746 | 3 | 3 | 3 |
| 127 | 748 | 3 | 3 | 3 |
| 138 | 759 | 3 | 3 | 3 |
| 139 | 760 | 3 | 3 | 3 |
| 143 | 764 | 3 | 3 | 3 |
| 146 | 767 | 3 | 3 | 3 |
| 148 | 769 | 3 | 3 | 3 |
| 149 | 770 | 3 | 3 | 3 |
| 168 | 789 | 3 | 3 | 3 |
| 169 | 790 | 3 | 3 | 3 |
| 173 | 794 | 3 | 3 | 3 |
| 174 | 795 | 3 | 3 | 3 |
| 175 | 796 | 3 | 3 | 3 |
| 176 | 797 | 3 | 3 | 3 |
| 177 | 798 | 2 | 2 | 2 |
| 180 | 801 | 3 | 3 | 3 |
| 186 | 807 | 3 | 3 | 3 |
| 187 | 808 | 2 | 2 | 2 |
| 188 | 809 | 2 | 3 | 3 |
| 203 | 824 | 3 | 3 | 3 |
| 204 | 825 | 3 | 3 | 3 |
| 225 | 846 | 3 | 3 | 3 |
| 234 | 855 | 3 | 3 | 3 |
| 256 | 877 | 3 | 3 | 3 |
| 312 | 933 | 2 | 2 | 2 |
| 313 | 934 | 3 | 3 | 3 |
| 314 | 935 | 3 | 3 | 3 |
| 315 | 936 | 3 | 3 | 3 |
| 316 | 937 | 3 | 3 | 3 |
| 317 | 938 | 3 | 3 | 3 |
| 341 | 962 | 3 | 3 | 3 |
| 345 | 966 | 3 | 3 | 3 |
| 346 | 967 | 3 | 3 | 3 |
| 349 | 970 | 3 | 3 | 3 |
| 350 | 971 | 3 | 3 | 3 |
| 356 | 977 | 3 | 3 | 3 |
| 2309 | 2930 | 3 | 3 | 3 |
| 2355 | 2976 | 2 | 2 | 2 |
| 2359 | 2980 | 3 | 3 | 3 |
| 2368 | 2989 | 2 | 2 | 2 |
| 2370 | 2991 | 2 | 2 | 2 |
| 2377 | 2998 | 3 | 3 | 3 |
| 2381 | 3002 | 3 | 3 | 3 |
| 2393 | 3014 | 3 | 3 | 3 |
| 2396 | 3017 | 2 | 2 | 2 |
| 2397 | 3018 | 3 | 3 | 3 |
| 2407 | 3028 | 3 | 3 | 3 |
| 2410 | 3031 | 3 | 3 | 3 |
| 2411 | 3032 | 3 | 3 | 3 |
| 2412 | 3033 | 3 | 3 | 3 |
| 2413 | 3034 | 2 | 2 | 2 |
| 2417 | 3038 | 2 | 2 | 3 |
| 2419 | 3040 | 3 | 3 | 3 |
| 2421 | 3042 | 3 | 3 | 3 |
| 2429 | 3050 | 2 | 3 | 3 |
| 2442 | 3063 | 3 | 3 | 3 |
| 2422 | 3043 | 3 | 3 | 3 |
| 2476 | 3097 | 3 | 3 | 3 |
| 2524 | 3145 | 3 | 3 | 3 |
| 2536 | 3157 | 3 | 3 | 3 |
| 2538 | 3159 | 3 | 3 | 3 |
| 2540 | 3161 | 3 | 3 | 3 |
| 2541 | 3162 | 3 | 3 | 3 |
| 2565 | 3186 | 3 | 3 | 3 |
| 2566 | 3187 | 3 | 3 | 3 |
| 2585 | 3206 | 3 | 3 | 3 |
| 2592 | 3213 | 2 | 2 | 2 |
| 2594 | 3215 | 2 | 3 | 3 |
| 2596 | 3217 | 3 | 3 | 3 |
| 2621 | 3242 | 3 | 3 | 3 |
| 2630 | 3251 | 3 | 3 | 3 |
| 2632 | 3253 | 3 | 3 | 3 |
| 2637 | 3258 | 3 | 3 | 3 |
| 2642 | 3263 | 2 | 2 | 2 |
| 2650 | 3271 | 3 | 3 | 3 |
| 2657 | 3278 | 3 | 3 | 3 |
| 2664 | 3285 | 2 | 2 | 2 |

Additional testing was carried on a larger panel of TREMs with the results summarized in FIG. 2. Each TREM, TREM core fragment, and TREM fragment was screened against three disease reporter constructions, each of which contained a premature termination codon (PTC): Factor IX-R²⁹TGA (B), Factor IX-Q44TAG (C), and RHO-S334TAA (D). The results from these screens are reported to the same scheme described above for Table 21, except only reporting results from the cell line corresponding to the type of stop codon designed to be recognized by the TREM.

### Example 8: Correction of a Missense Mutation in an ORF with Administration of a TREM

This example describes the administration of a TREM to correct a missense mutation. In this example, a TREM translates a reporter with a missense mutation into a wild type (WT) protein by incorporation of the WT amino acid (at the missense position) in the protein.

### Host Cell Modification

A cell line stably expressing a GFP reporter construct containing a missense mutation, for example T2031 or E222G, which prevent GFP excitation at the 470 nm and 390 nm wavelengths, is generated using the FlpIn system according to manufacturer's instructions. Briefly, HEK293T (293T ATCC® CRL-3216) cells are co-transfected with an expression vector containing a GFP reporter with a missense mutation, such as pcDNA5/FRT-NanoLuc-TAA and a pOG44 Flp-Recombinase expression vector using Lipofectamine2000 according to manufacturer's instructions. After 24 hours, the media is replaced with fresh media. The next day, the cells are split 1:2 and selected with 100 ug/mL Hygromycin for 5 days. The remaining cells are expanded and tested for reporter construct expression.

### Synthesis and Preparation of TREM

The TREM is synthesized as described in Example 1 and quality control methods as described in Examples 2-5 are performed. To ensure proper folding, the TREM is heated at 85° C. for 2 minutes and then snap cooled at 4° C. for 5 minutes.

Transfection of Non-Cognate TREM into Host Cells

To deliver the TREM to mammalian cells, 100 nM of TREM is transfected into cells expressing the ORF having a missense mutation using lipofectamine 2000 reagents according to the manufacturer's instructions. After 6-18 hours, the transfection media is removed and replaced with fresh complete media.

### Missense Mutation Correction Assay

To monitor the efficacy of the TREM to correct the missense mutation in the reporter construct, 24-48 hours after TREM transfection, cell media is replaced, and cell fluorescence is measured. As a negative control, no TREM is transfected in the cells and as a positive control, cells expressing WT GFP are used for this assay. If the TREM is functional, it is expected that the GFP protein produced fluoresces when illuminated with a 390 nm excitation wavelength using a fluorimeter, as observed in the positive control. If the TREM is not functional, the GFP protein produced fluoresces only when excited with a 470 nm wavelength, as is observed in the negative control.

### Example 9: Evaluation of Protein Expression Levels of SMC-Containing ORF with Administration of a TREM

This example describes administration of a TREM to alter expression levels of an SMC-containing ORF.

To create a system in which to study the effects of TREM administration on protein expression levels of an SMC-containing protein, in this example, from the PNPL3A gene coding for adiponutrin, a plasmid containing the PNPL3A rs738408 ORF sequence is transfected in the normal human hepatocyte cell line THLE-3, edited by CRISPR/Cas to contain a frameshift mutation in a coding exon of PNPLA3 to knock out endogenous PNPLA3 (THLE-3_PNPLA3KO cells). As a control, an aliquot of THLE-3_PNPLA3KO cells are transfected with a plasmid containing the wildtype PNPL3A ORF sequence.

### Synthesis and Preparation of TREM

An arginine TREM is synthesized as described in Example 1 and quality control methods as described in Examples 2-5 are performed. To ensure proper folding, the TREM is heated at 85° C. for 2 minutes and then snap cooled at 4° C. for 5 minutes.

### Evaluation of Protein Level of SMC-Containing ORF

A TREM is delivered to the THLE-3_PNPLA3KO cells containing the rs738408 ORF sequence as well as to the THLE-3_PNPLA3KO cells containing the wildtype PNPLA3 ORF sequence. In this example, the TREM contains a proline isoacceptor containing an AGG anticodon, that base pairs to the CCT codon, i.e. with the sequence GGCUCGUUGGUCUAGGGGUAUGAUUCUCGCUUAGGGUGCGAGAGGUCCCGGGUU

CAAAUCCCGGACGAGCCC. A time course is performed ranging from 30 minutes to 6 hours with hour-long interval time points. At each time point, cells are trypsinized, washed and lysed. Cell lysates are analyzed by Western blotting and blots are probed with antibodies against the adiponutrin protein. A total protein loading control, such as GAPDH, actin or tubulin, is also probed as a loading control.

The methods described in this example can be adopted for use to evaluate the expression levels of the adiponutrin protein in rs738408 ORF containing cells.

### Example 10: Modulation of Protein Translation Rate of SMC-Containing ORF with TREM Administration

This example describes administration of a TREM to alter the rate of protein translation of an SMC-containing ORF.

To monitor the effects of TREM addition on translation elongation rates, an in vitro translation system, in this example the RRL system from Promega, is used in which the fluorescence change over time of a reporter gene, in this example GFP, is a surrogate for translation rates.

### Synthesis and Preparation of TREM

An arginine TREM is synthesized as described in Example 1 and quality control methods as described in Examples 2-5 are performed. To ensure proper folding, the TREM is heated at 85° C. for 2 minutes and then snap cooled at 4° C. for 5 minutes.

### Evaluation of Protein Translation Rate of SMC-Containing ORF

First, a rabbit reticulocyte lysate that is depleted of the endogenous tRNA using an antisense oligonucleotide targeting the sequence between the anticodon and variable loop is generated (see, e.g., Cui et al. 2018. Nucleic Acids Res. 46 (12): 6387-6400). In this example, a TREM comprising an alanine isoacceptor containing an UGC anticodon, that base pairs to the GCA codon, i.e. with the sequence GGGGAUGUAGCUCAGUGGUAGAGCGCAUGCUUUGCAUGUAUGAGGUCCCGGGUU CGAUCCCCGGCAUCUCCA is added to the in vitro translation assay lysate in addition to 0.1-0.5 ug/uL of mRNA coding for the wildtype TERT ORF fused to the GFP ORF by a linker or an mRNA coding for the rs2736098 TERT ORF fused to the GFP ORF by a linker. The progress of GFP mRNA translation is monitored by fluorescence increase on a microplate reader at 37° C. using Mex485/hem528 with data points collected every 30 seconds over a period of 1 hour. The amount of fluorescence change over time is plotted to determine the rate of translation elongation of the wildtype ORF compared to the rs2736098 ORF with and without TREM addition. The methods described in this example can be adopted for use to evaluate the translation rate of the rs2736098 ORF and the wildtype ORF in the presence or absence of TREM.

### Example 11: Rescue of Full-Length GLA Expression and Activity in Fabry Patient Fibroblasts Upon Administration of an Exemplary TREM

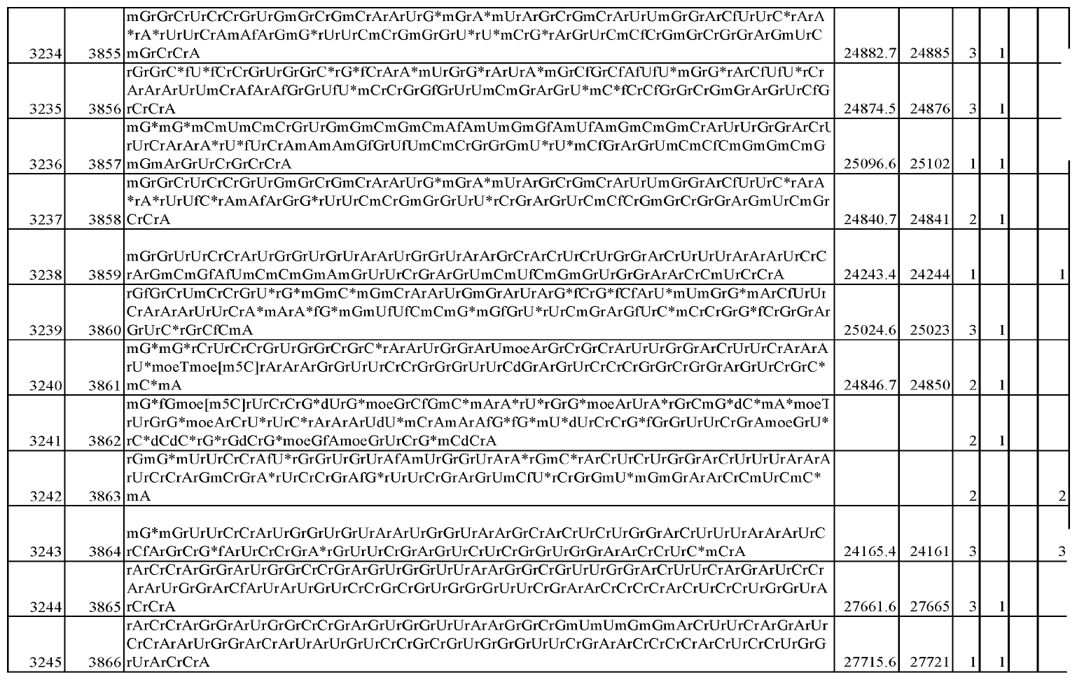

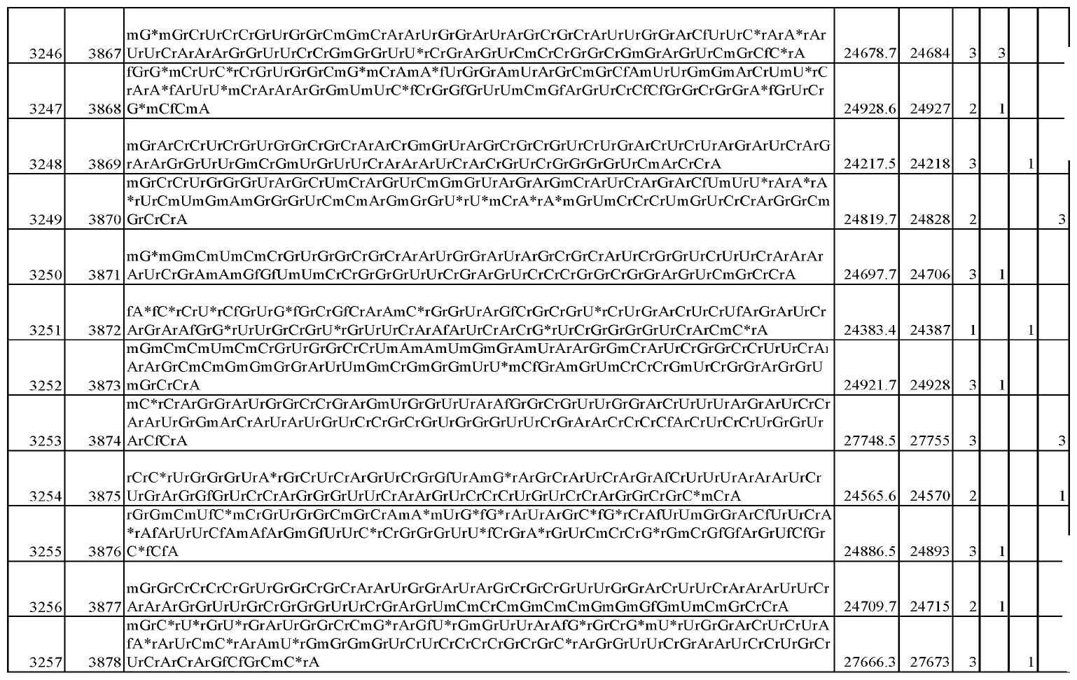

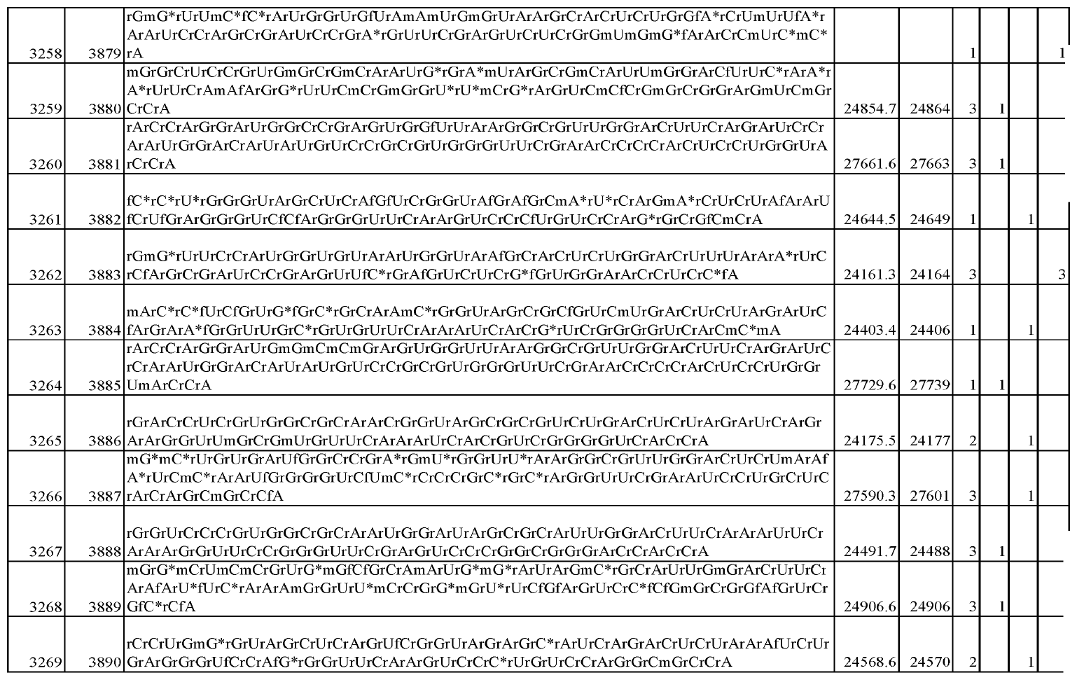

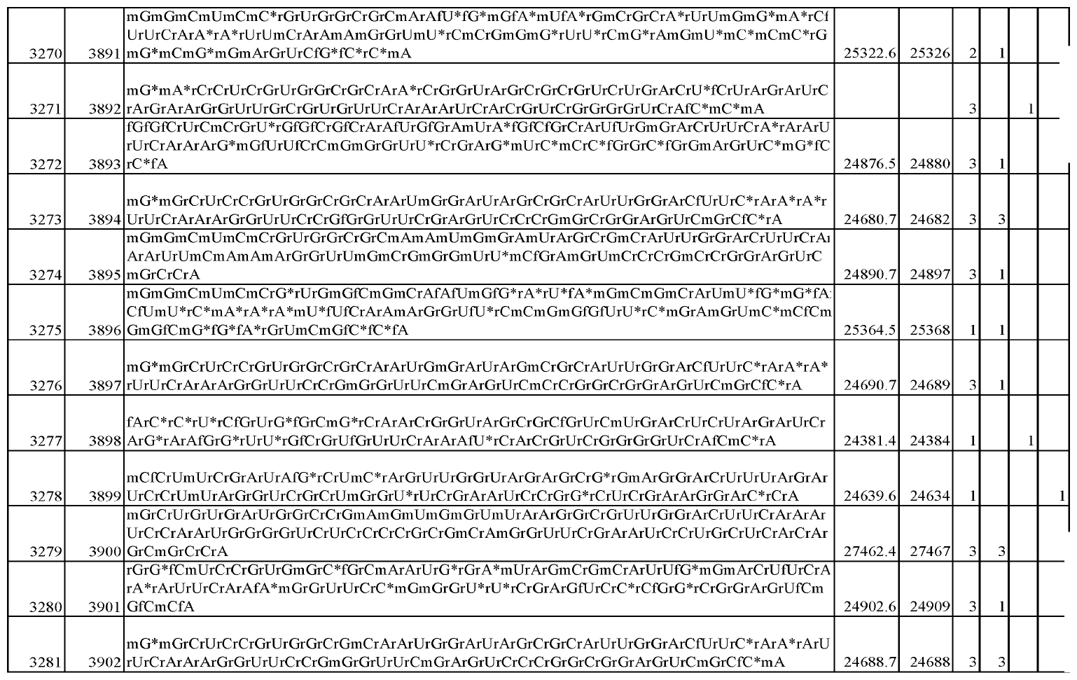

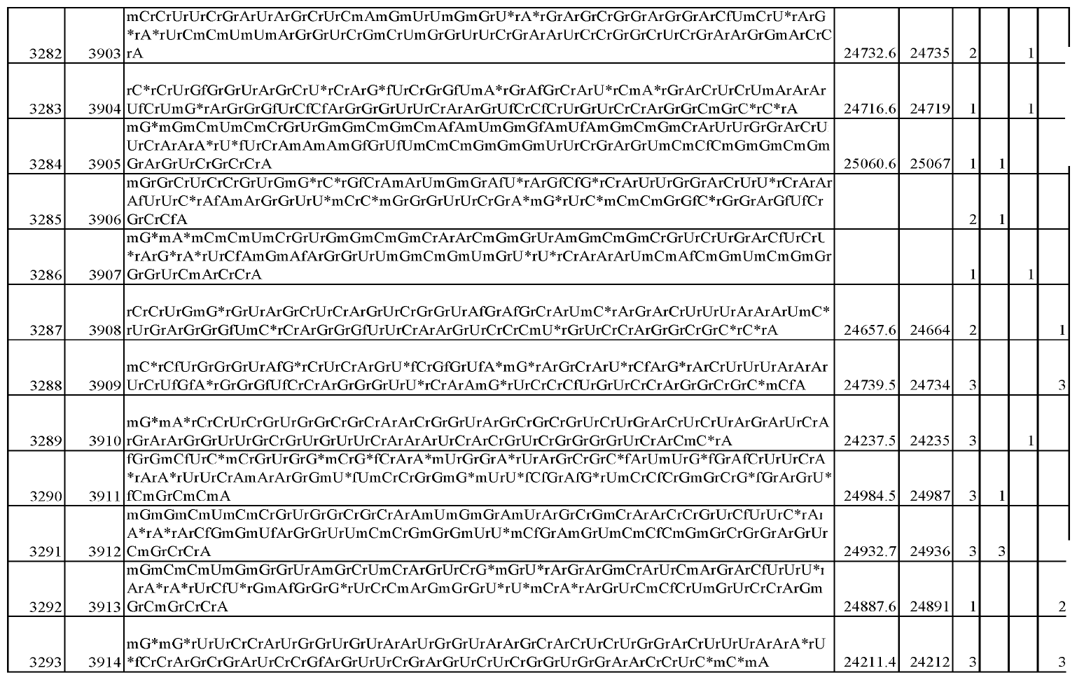

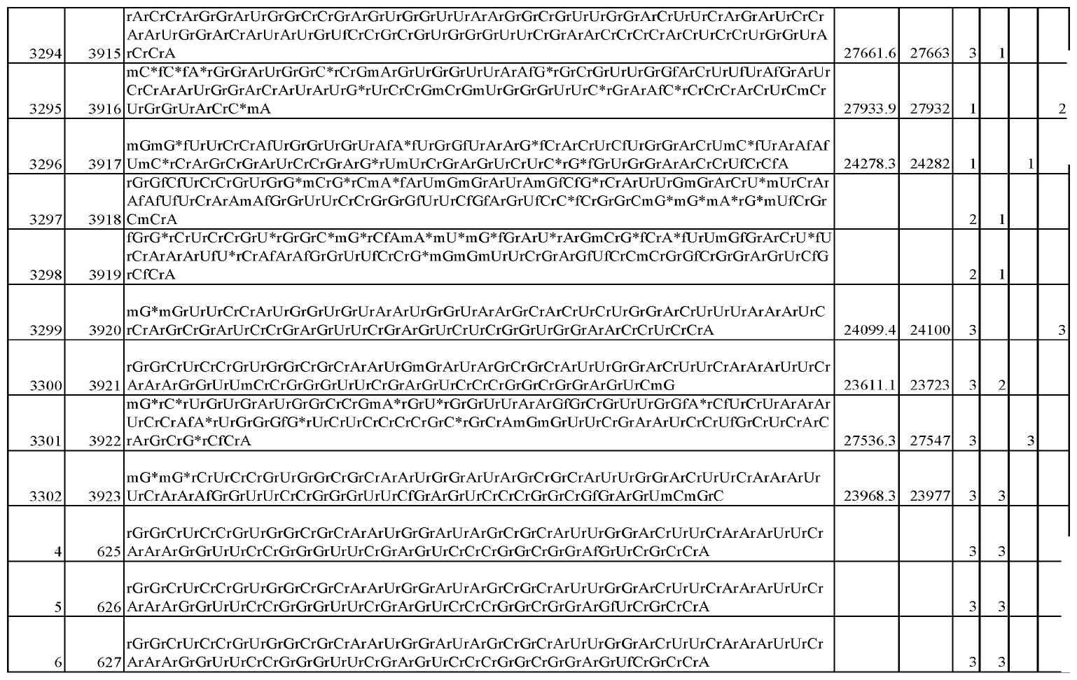

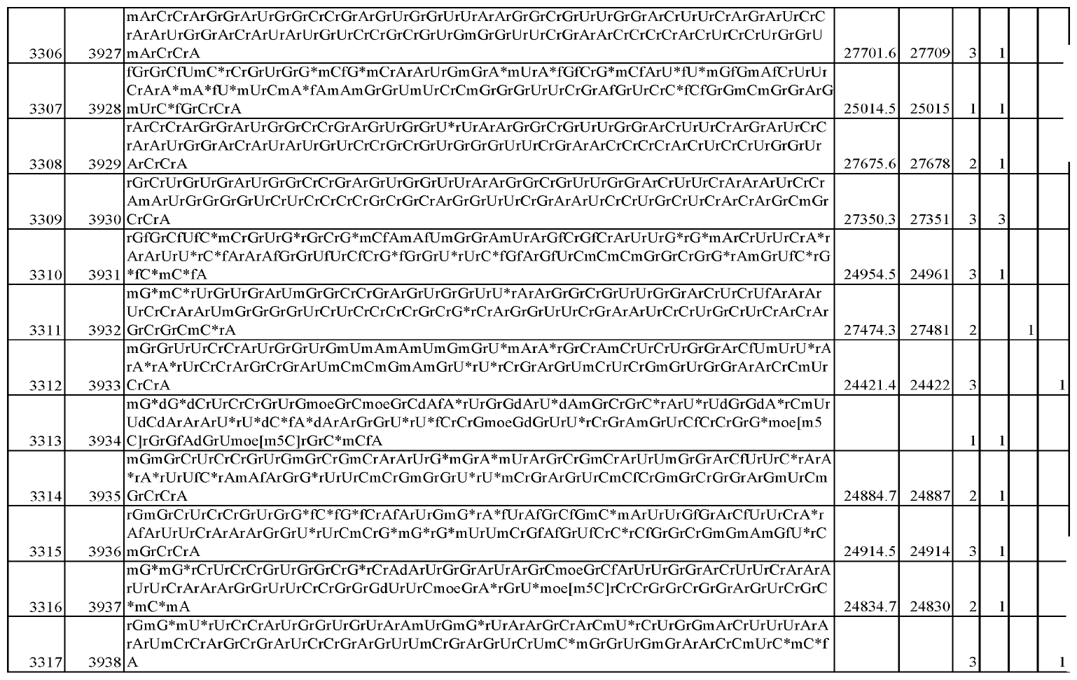

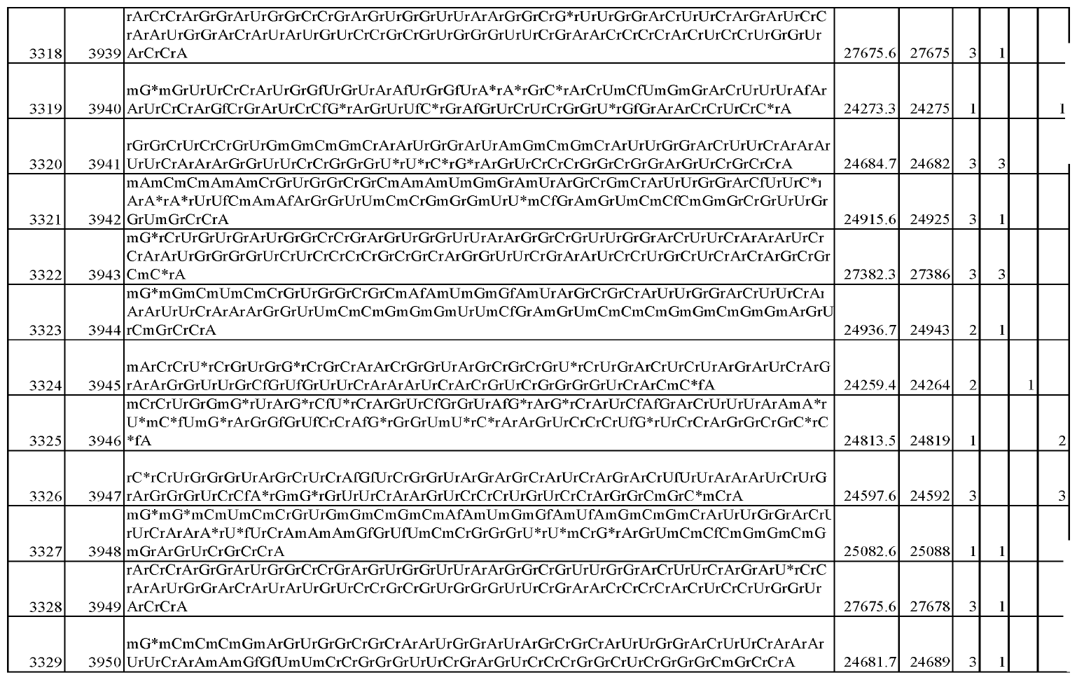

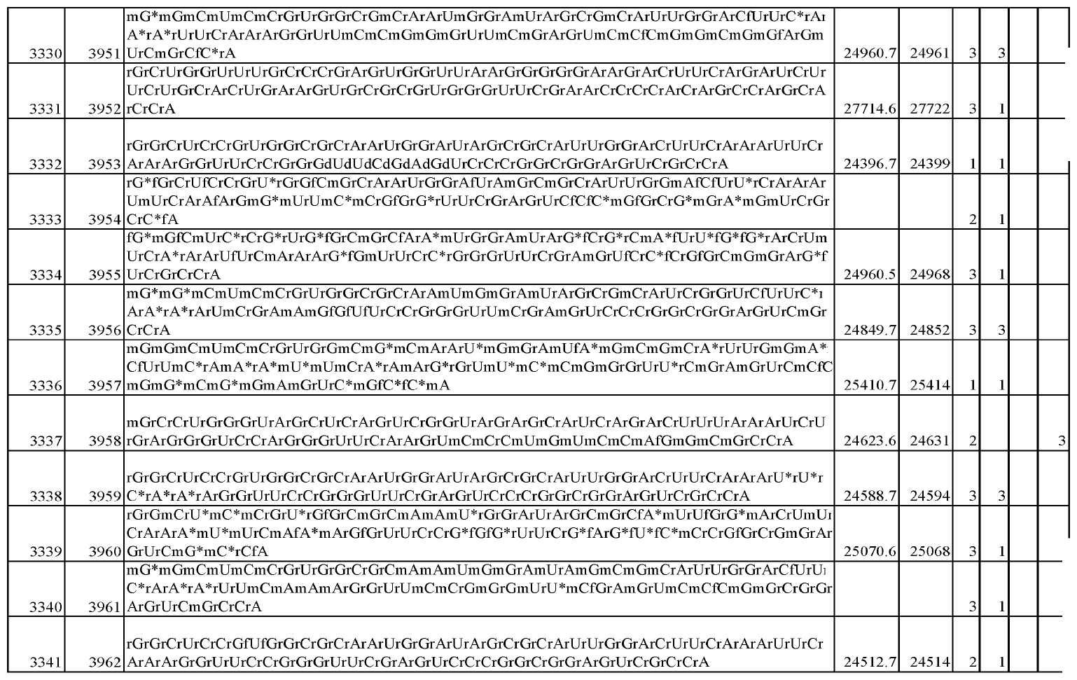

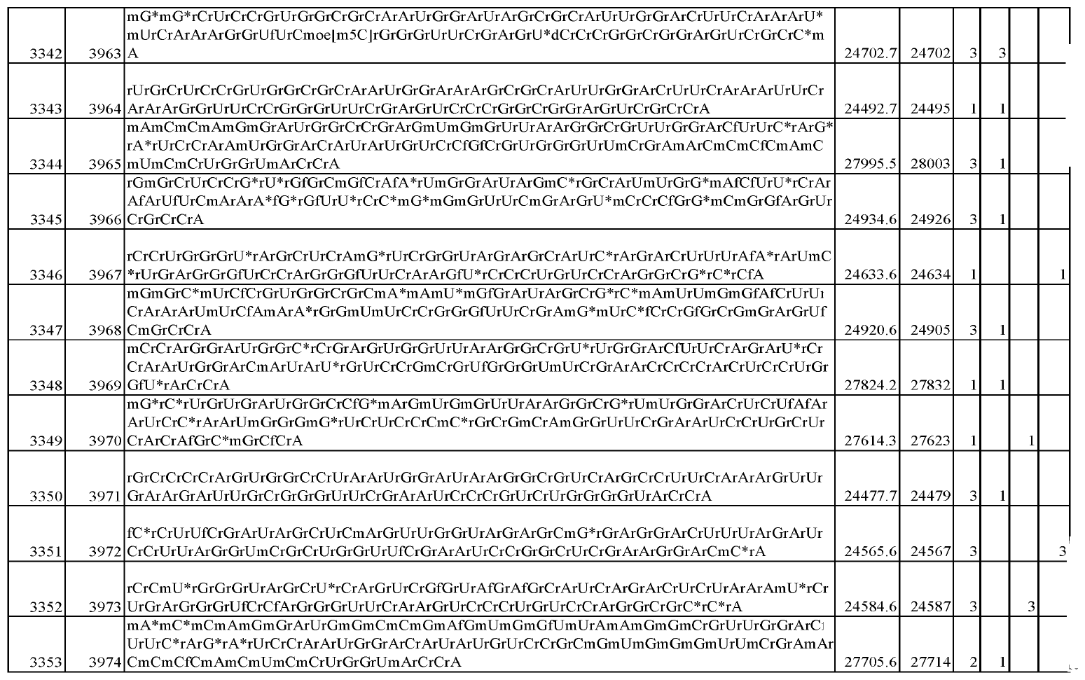

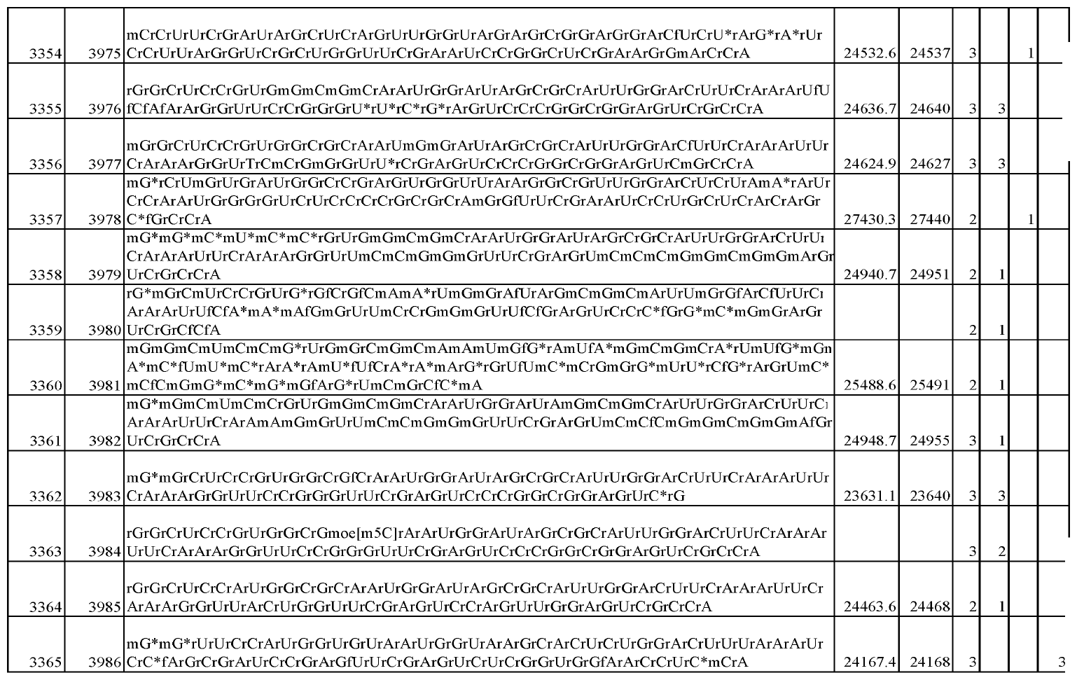

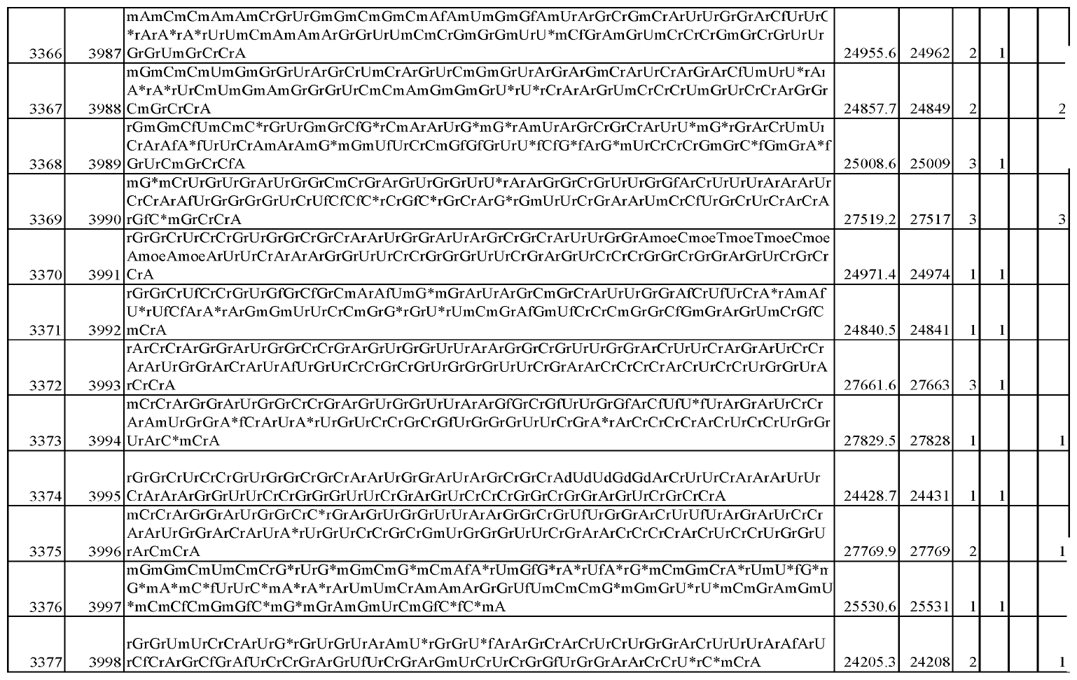

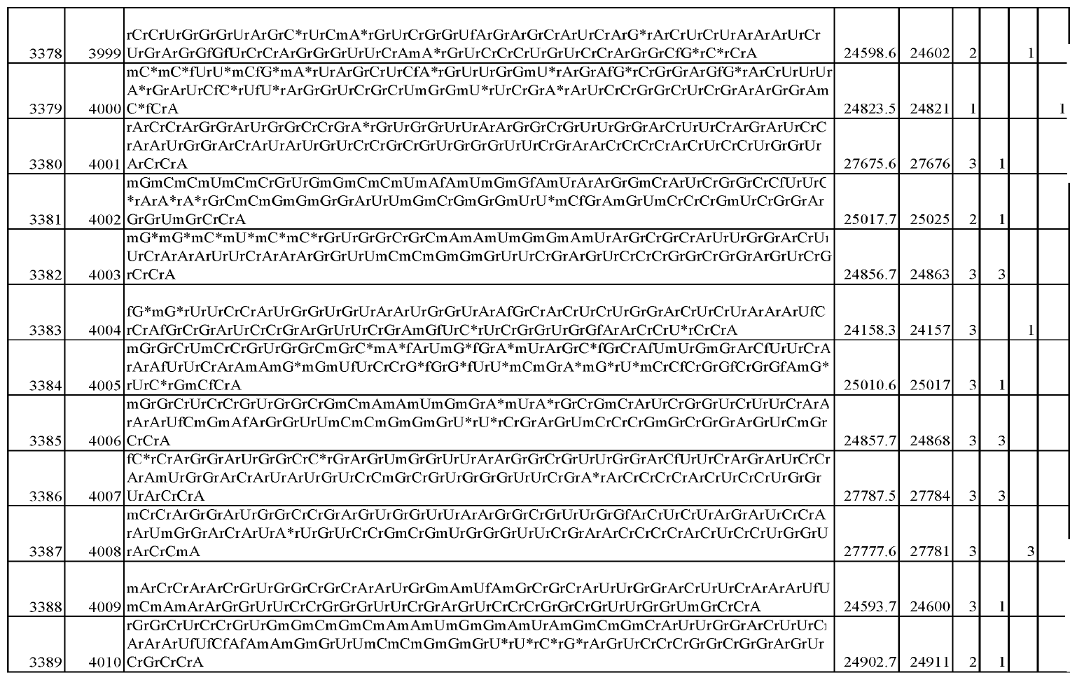

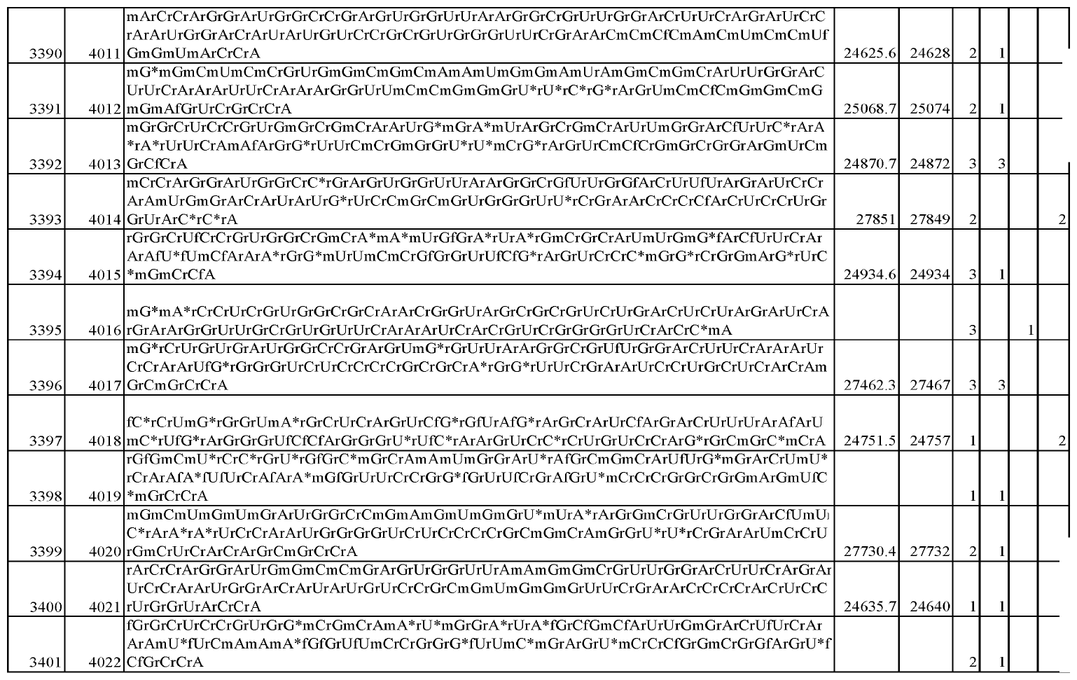

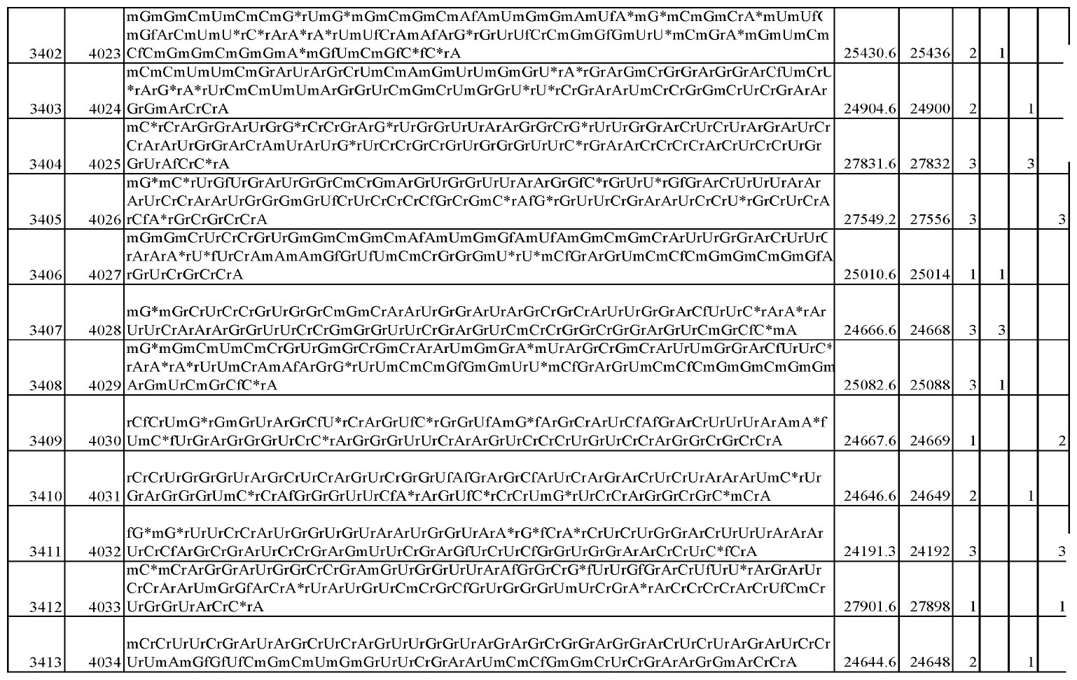

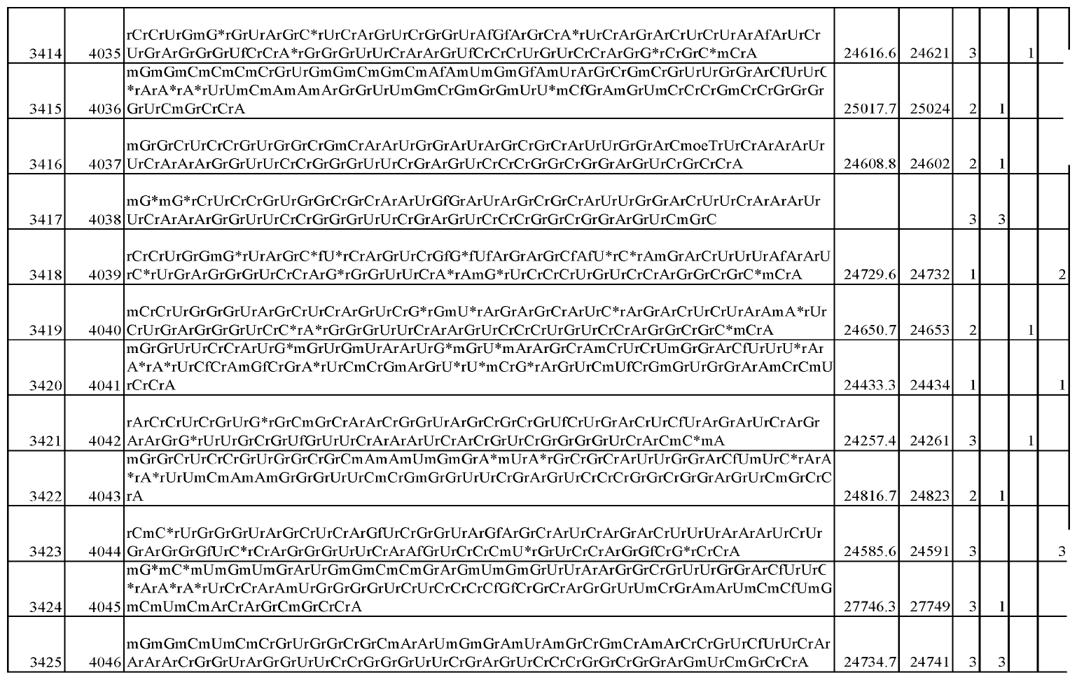

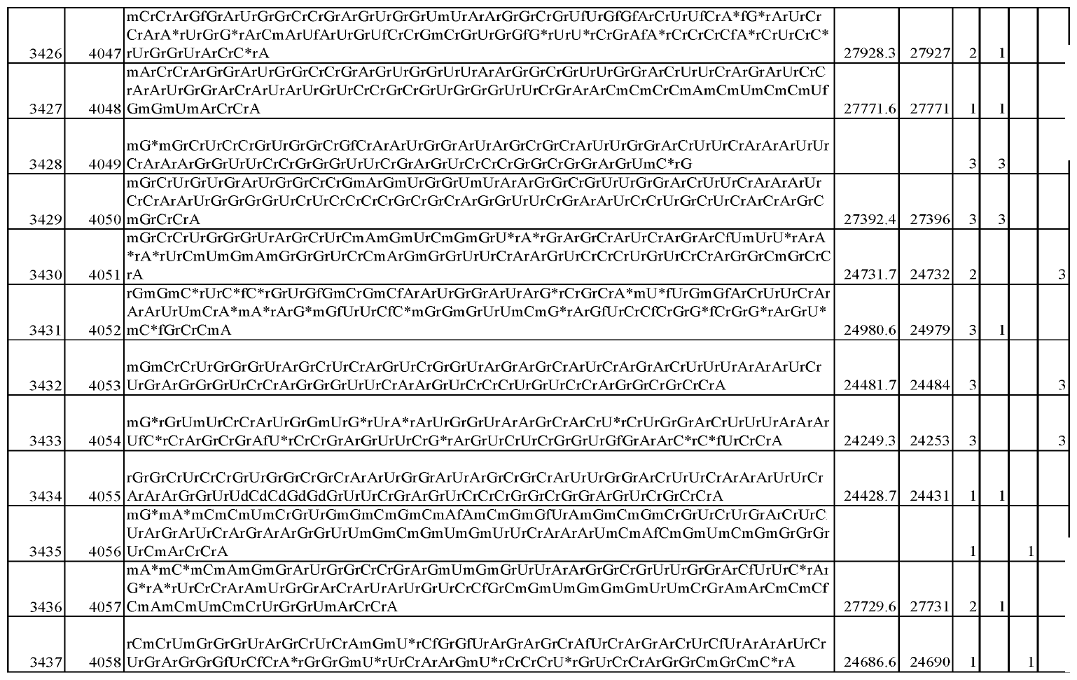

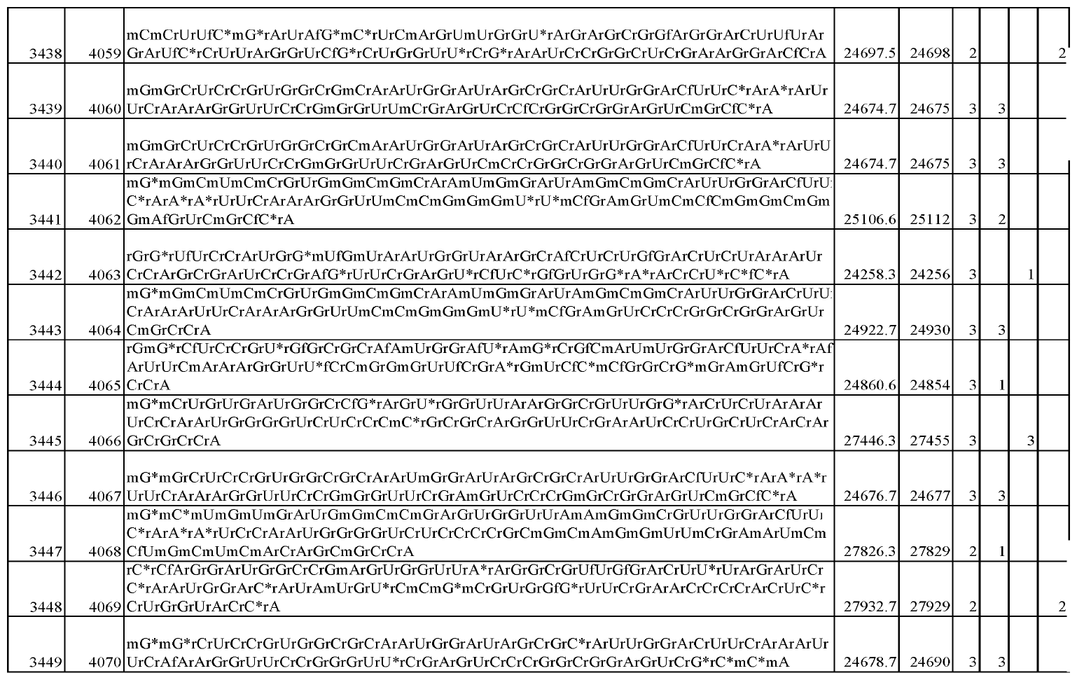

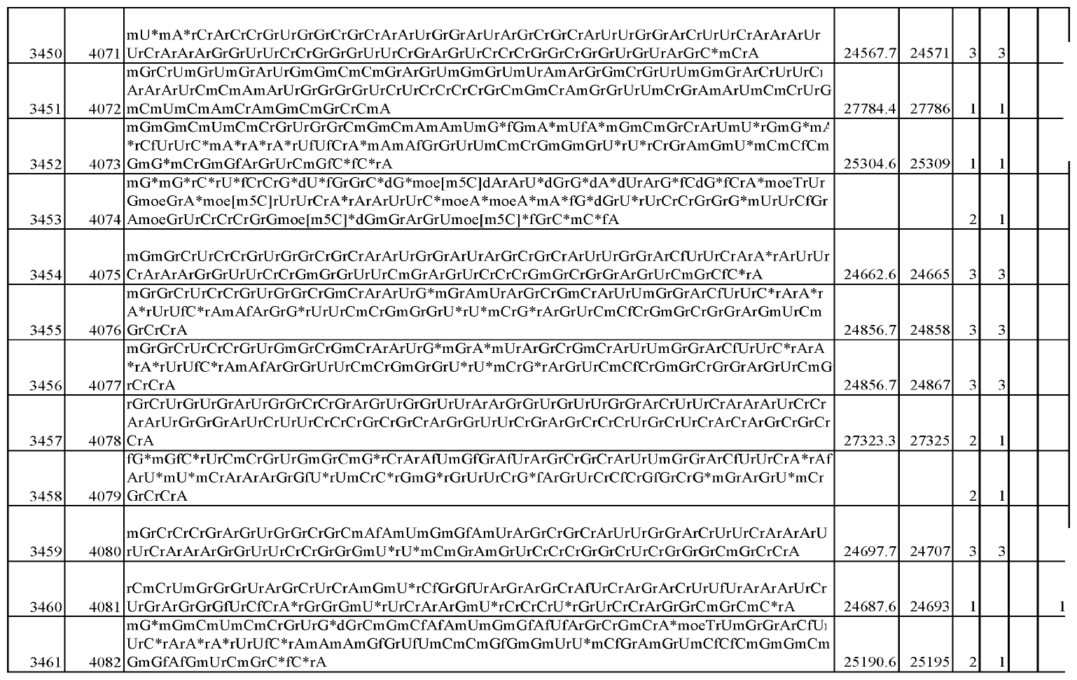

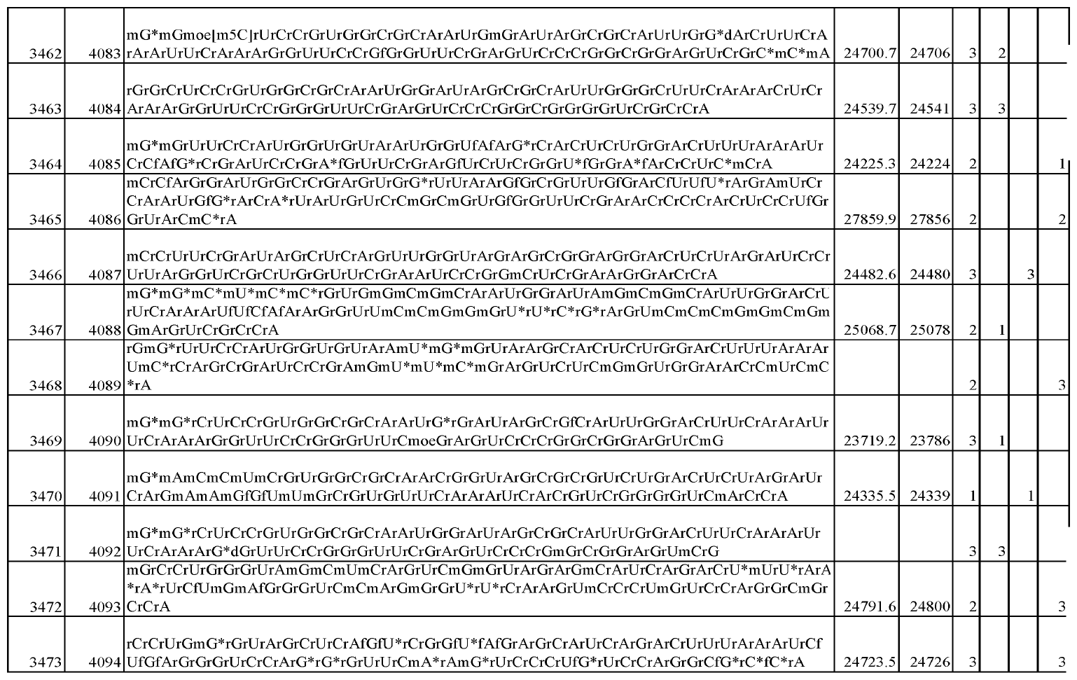

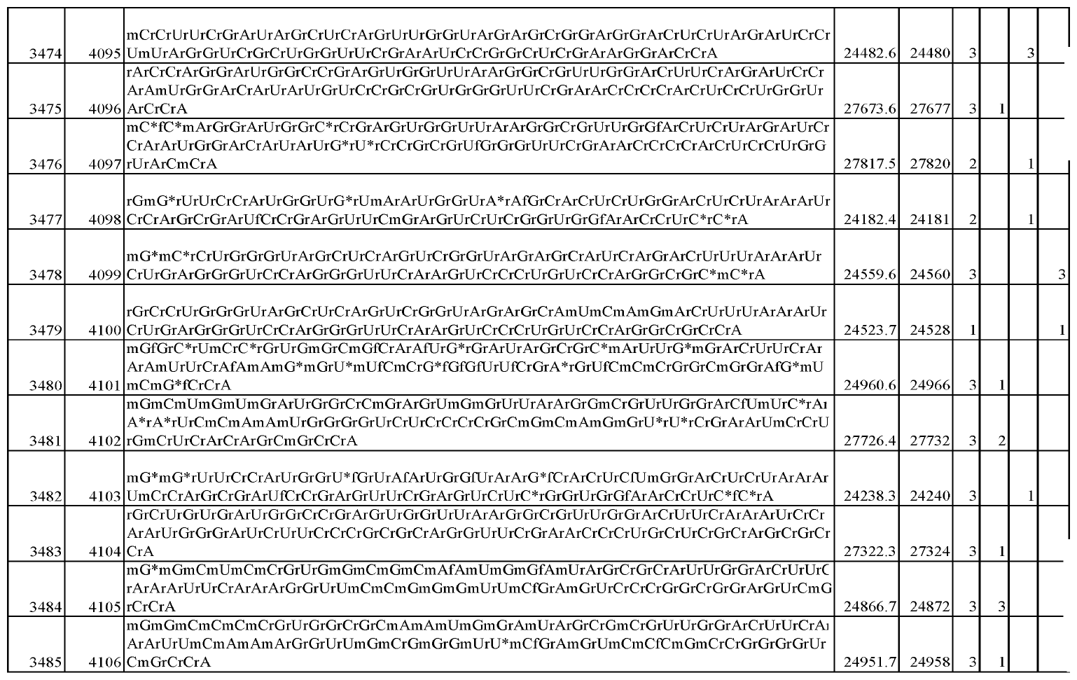

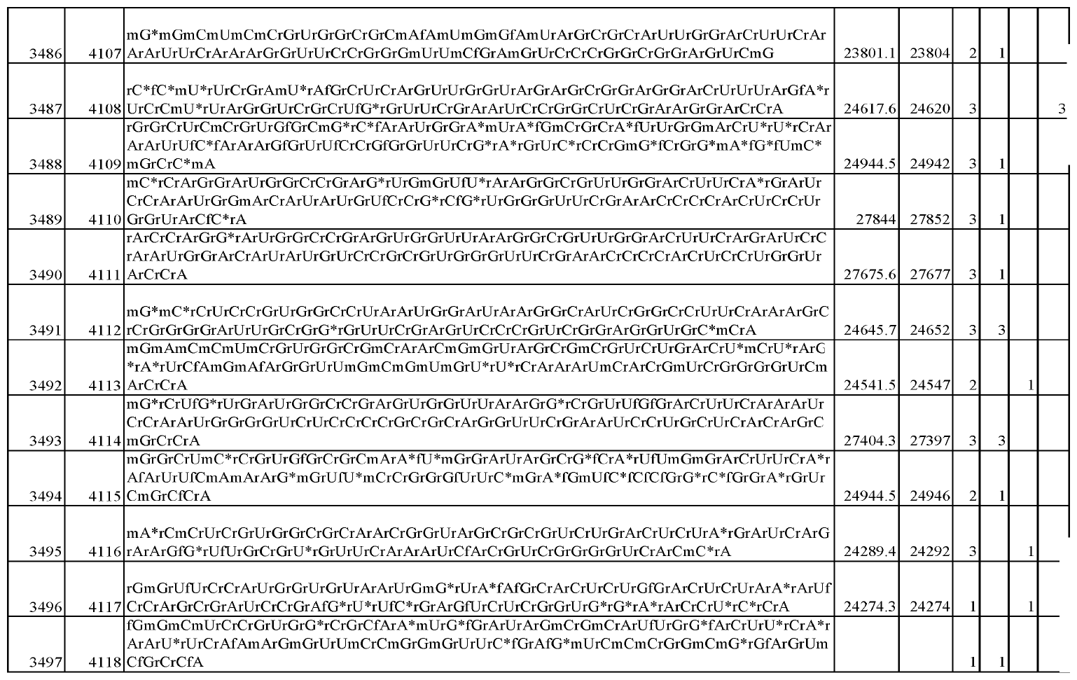

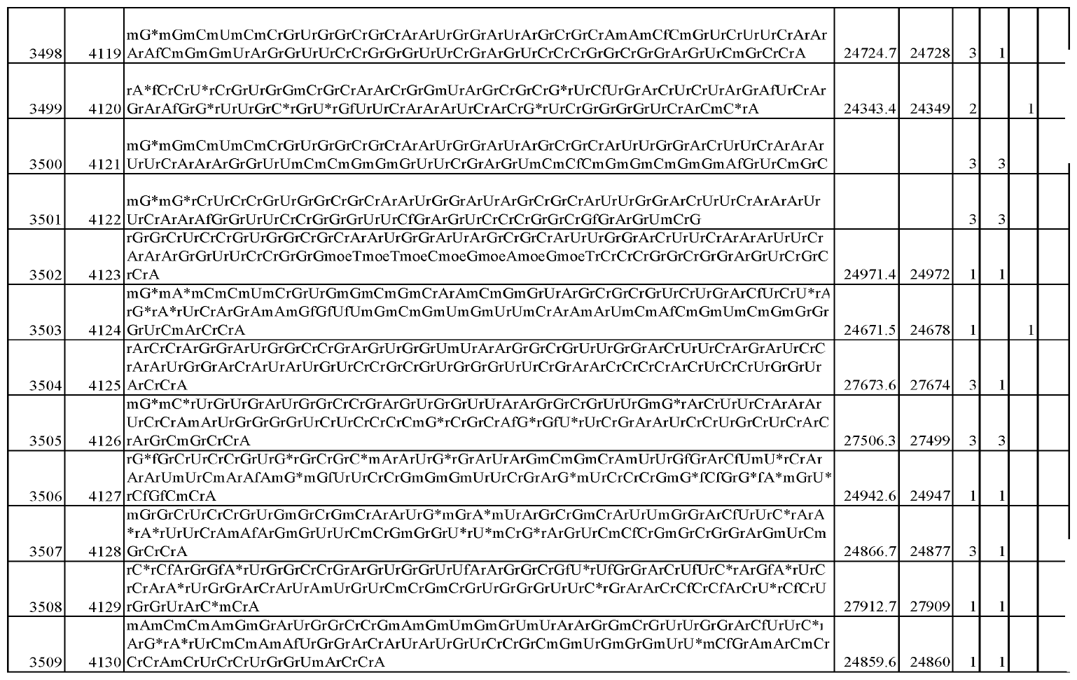

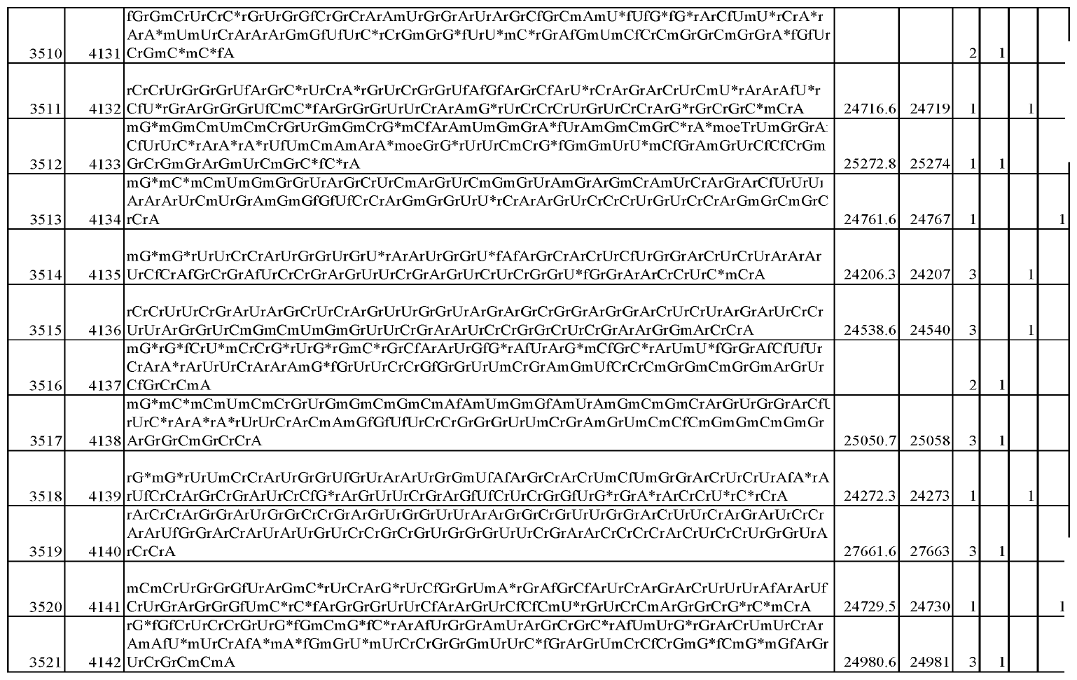

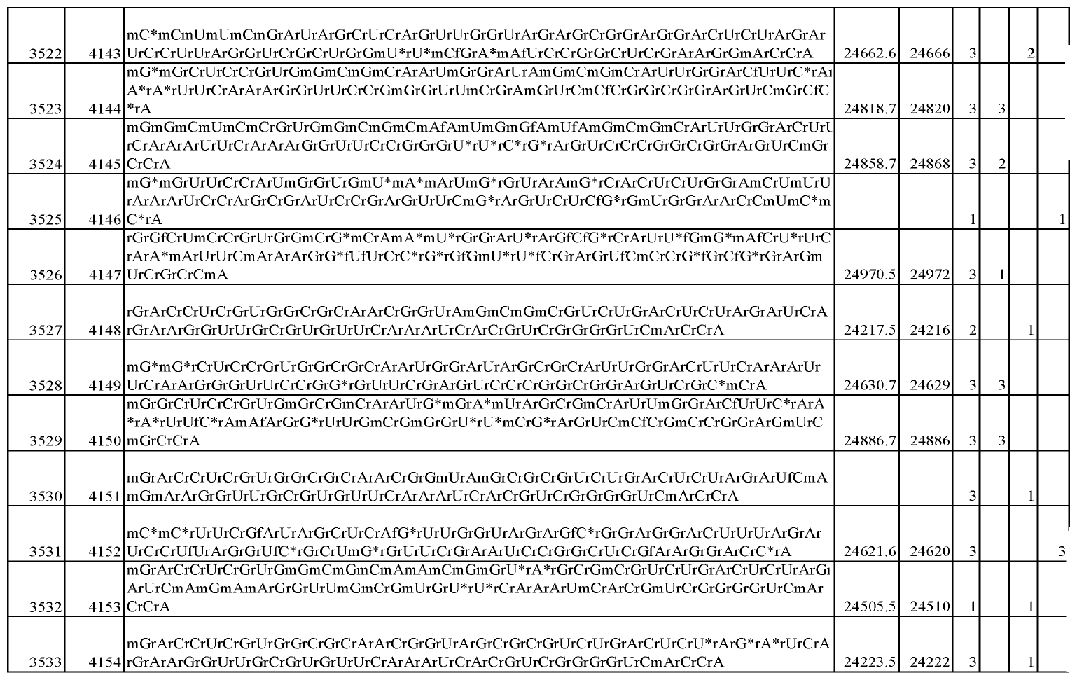

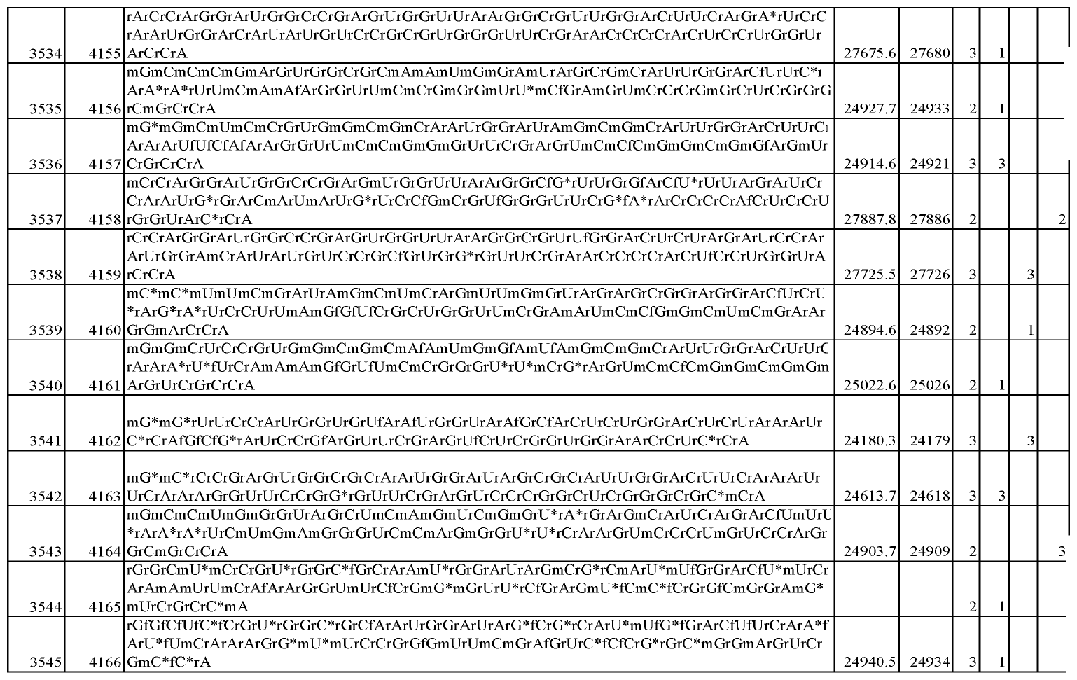

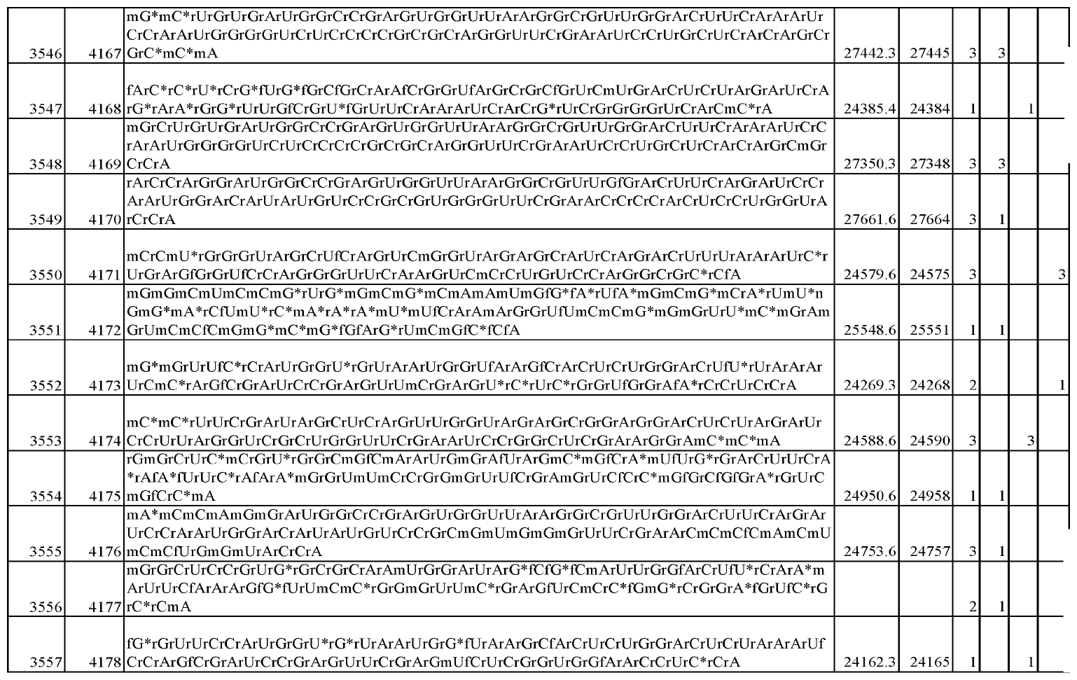

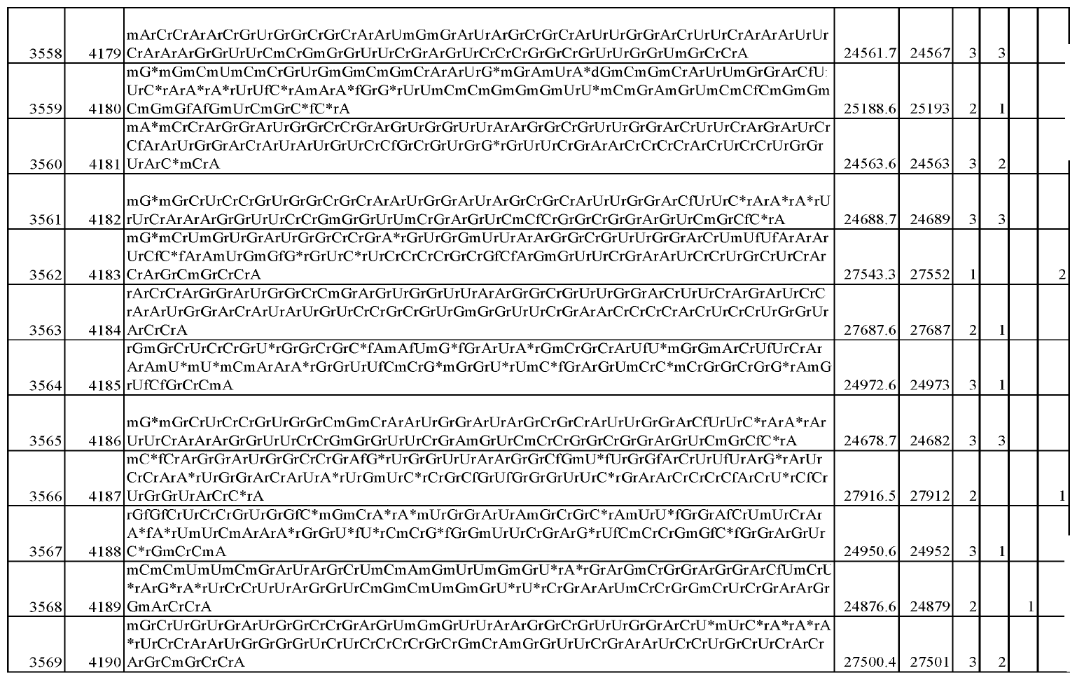

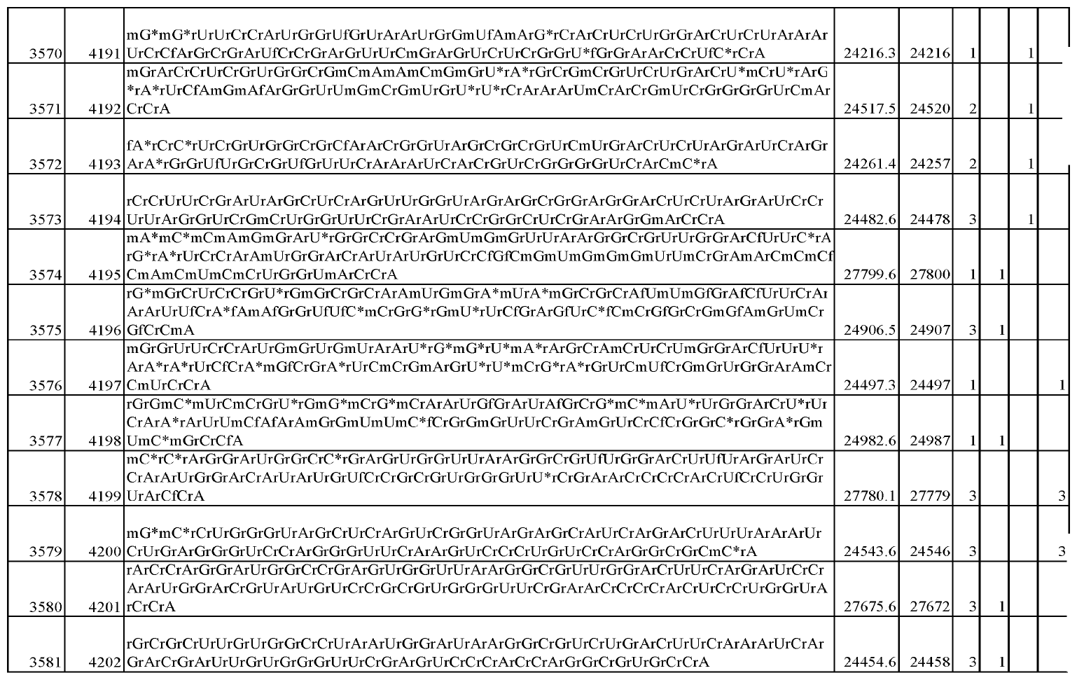

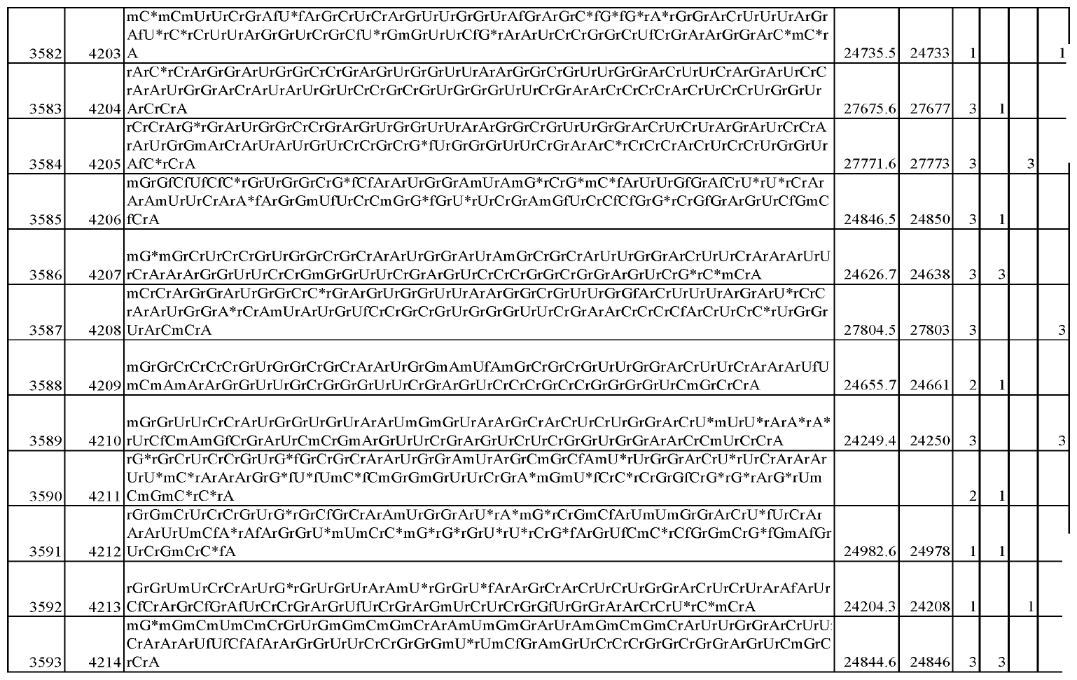

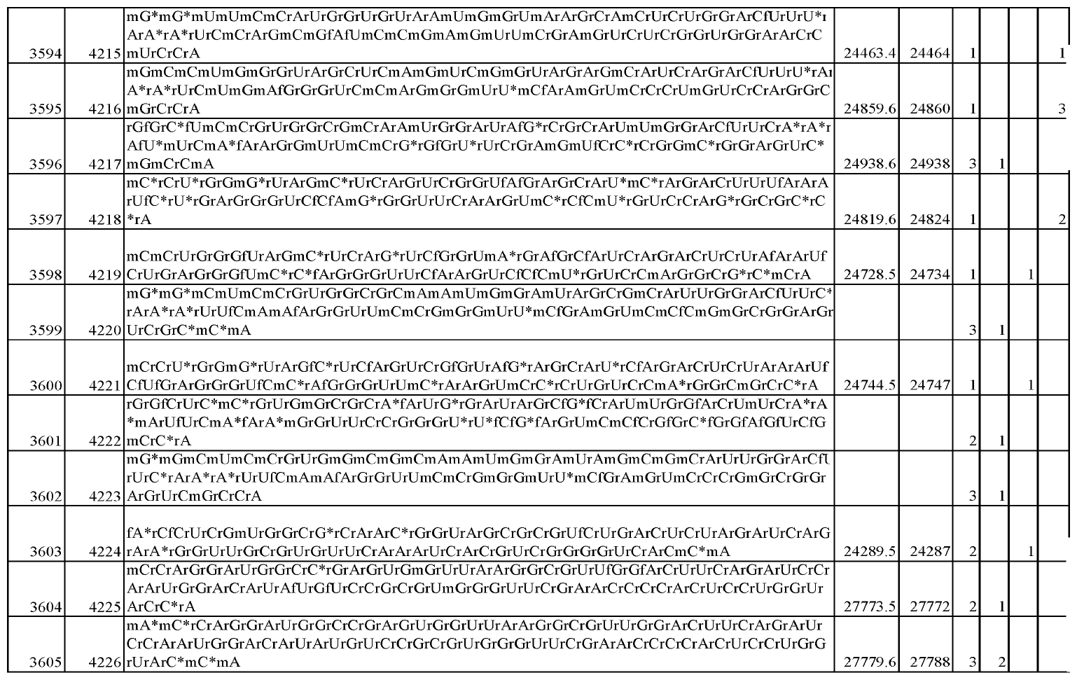

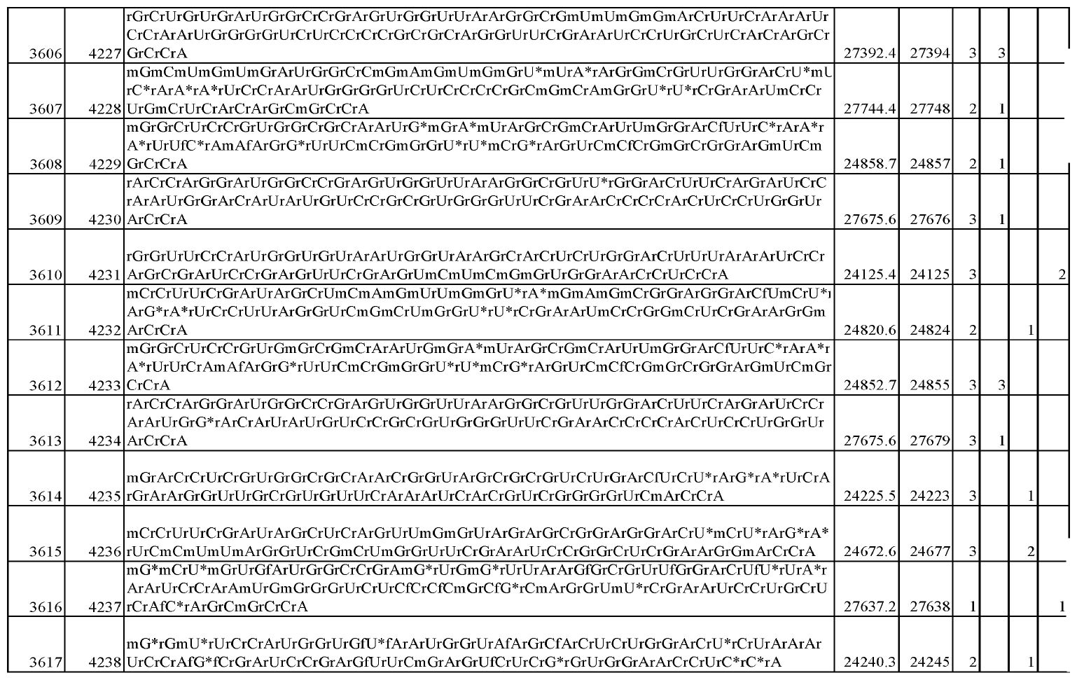

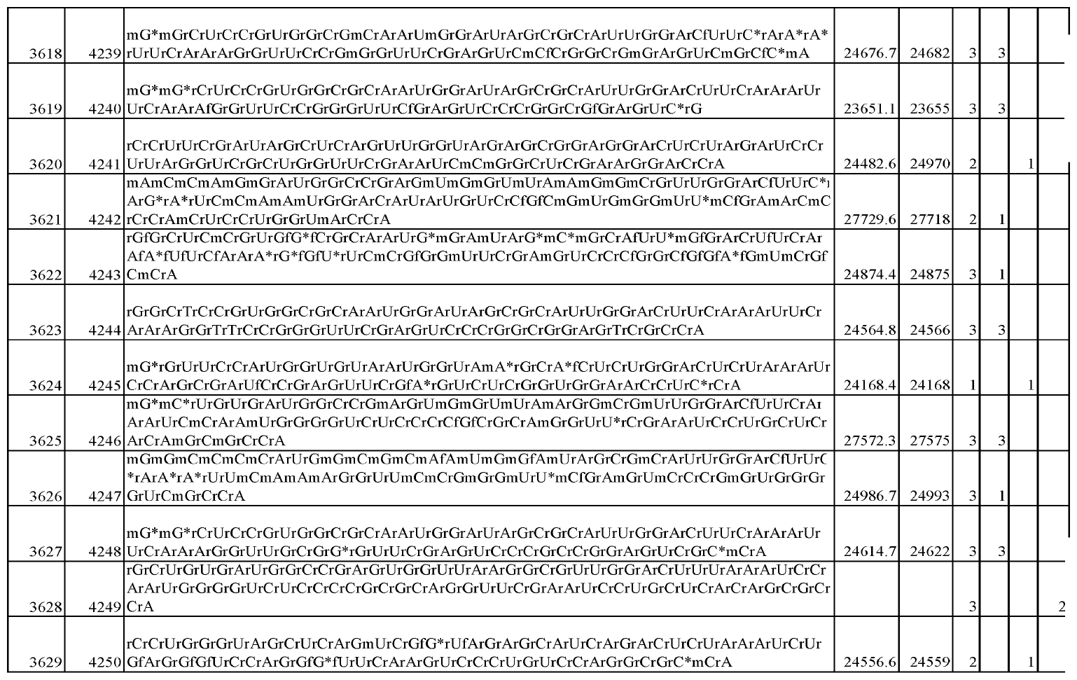

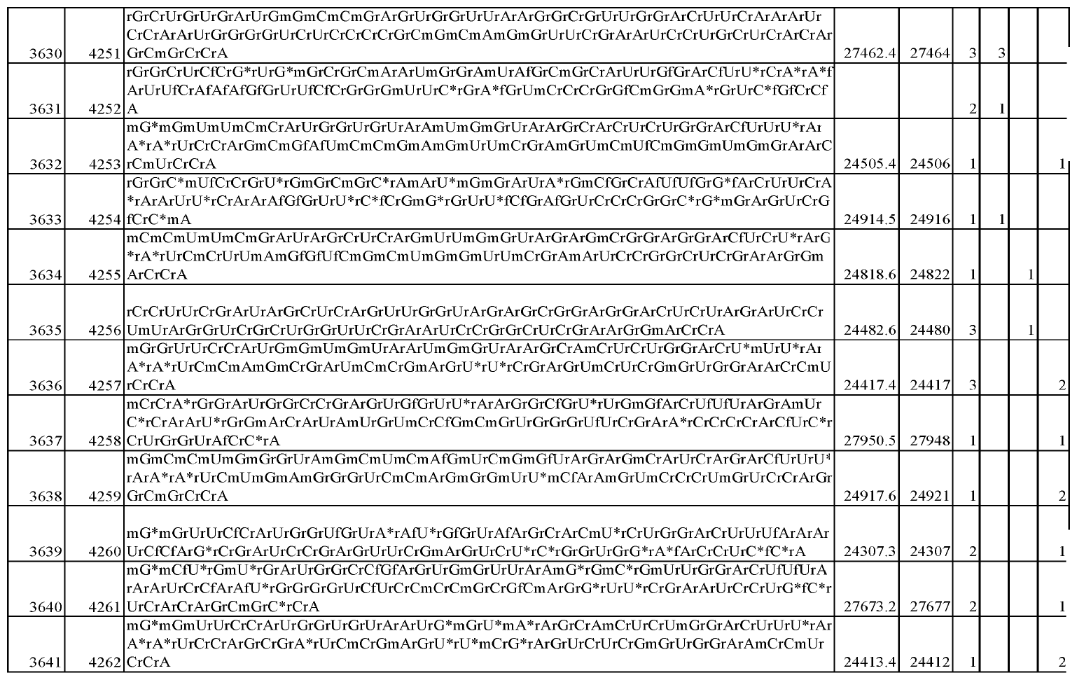

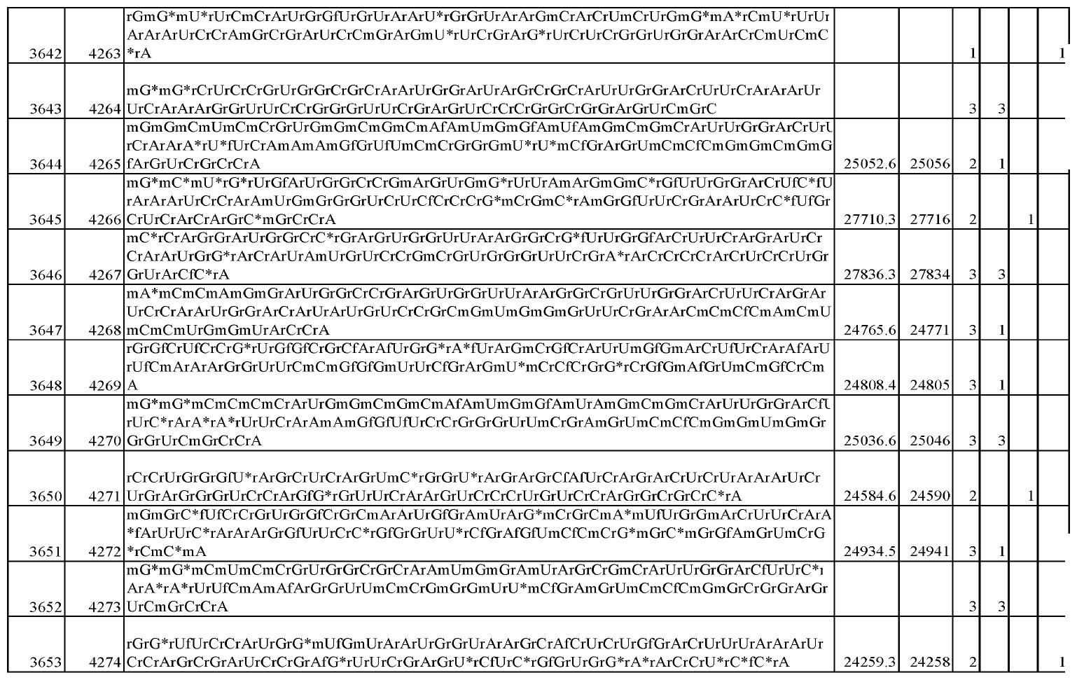

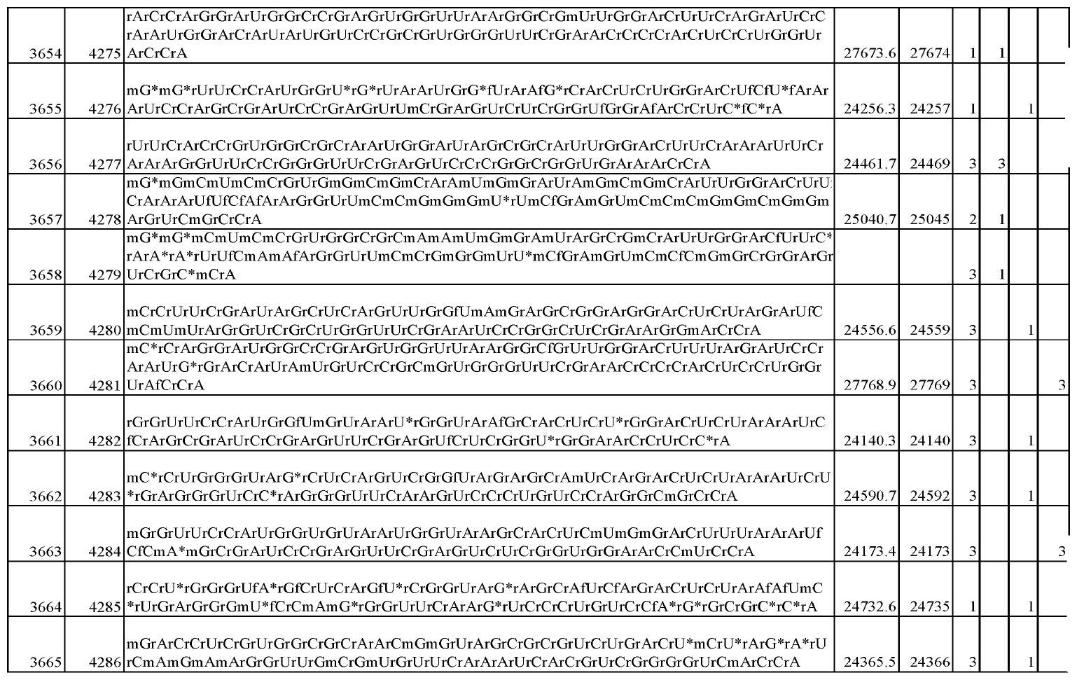

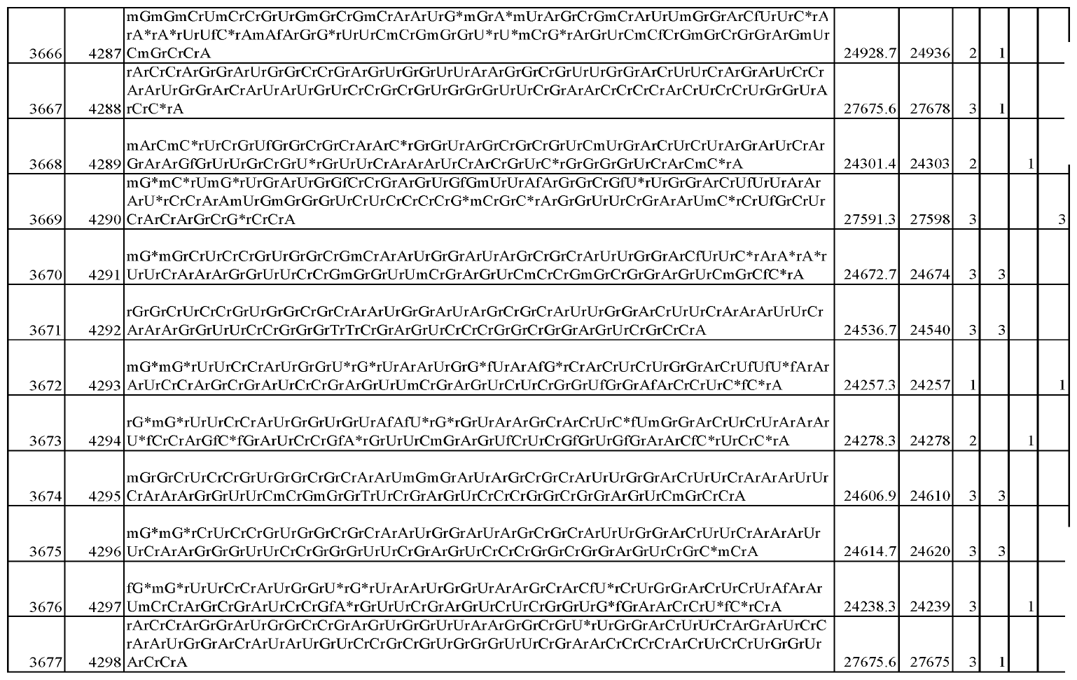

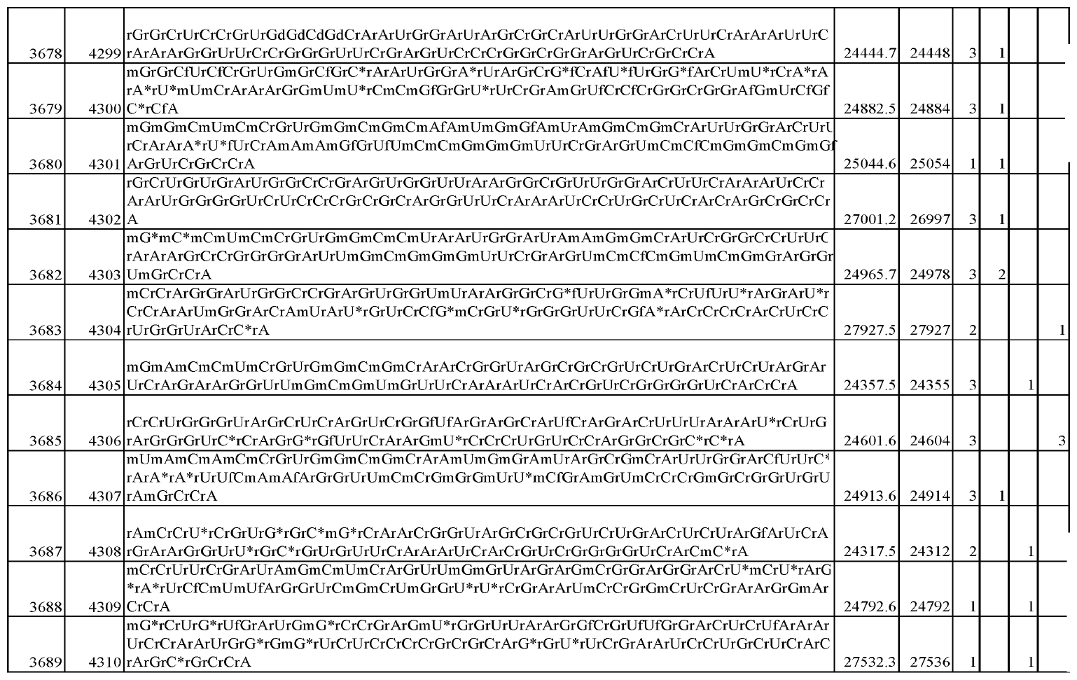

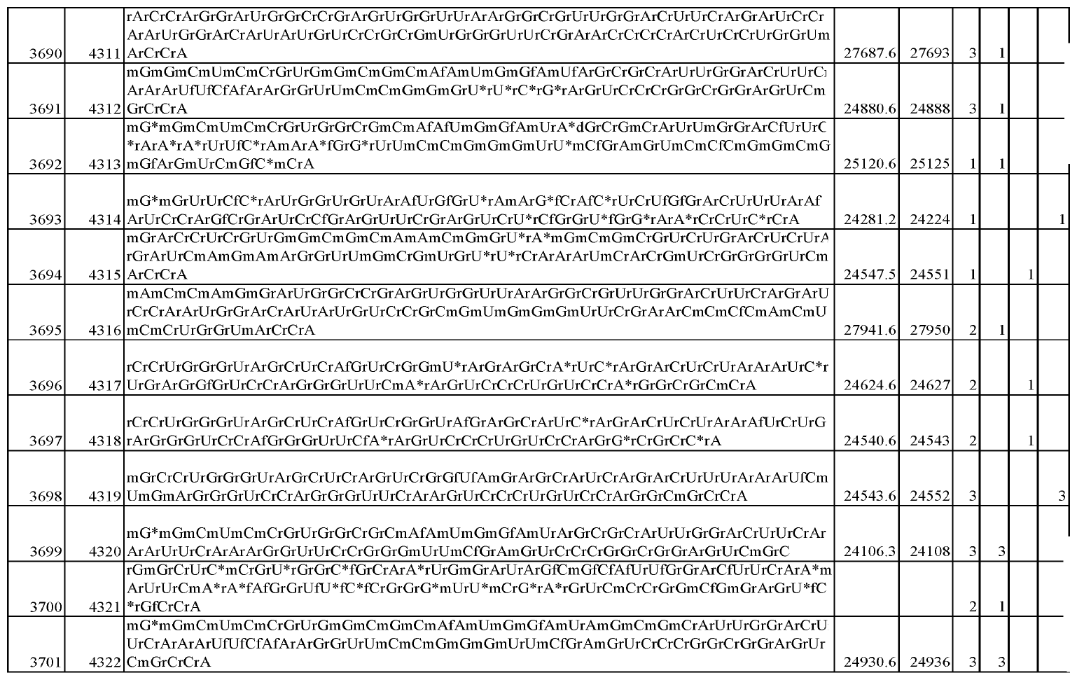

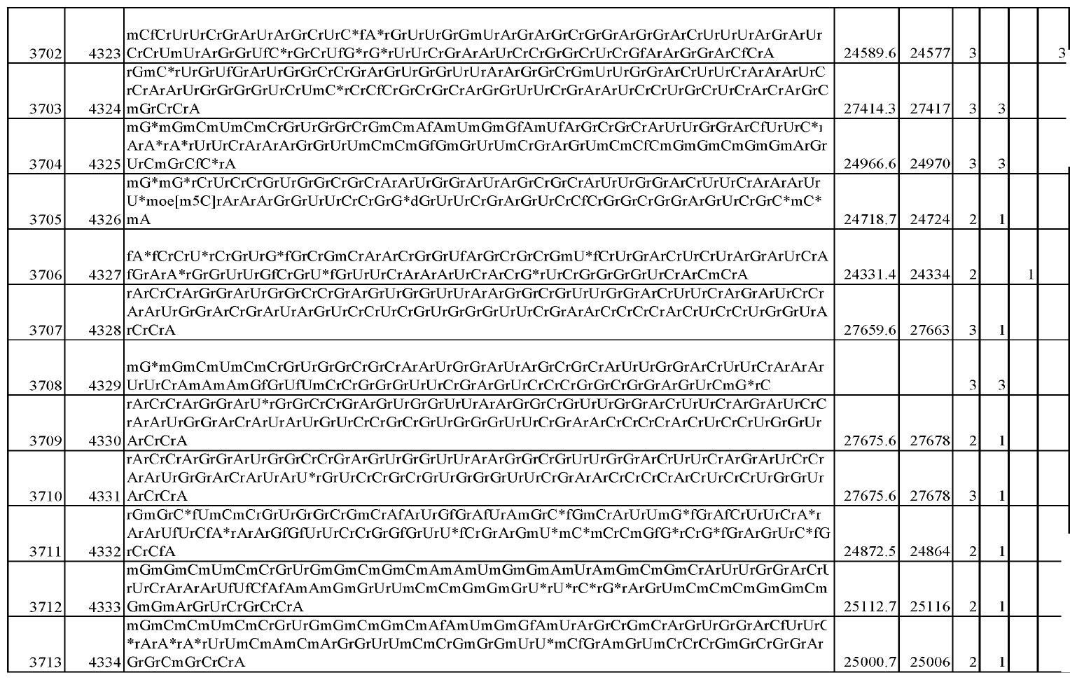

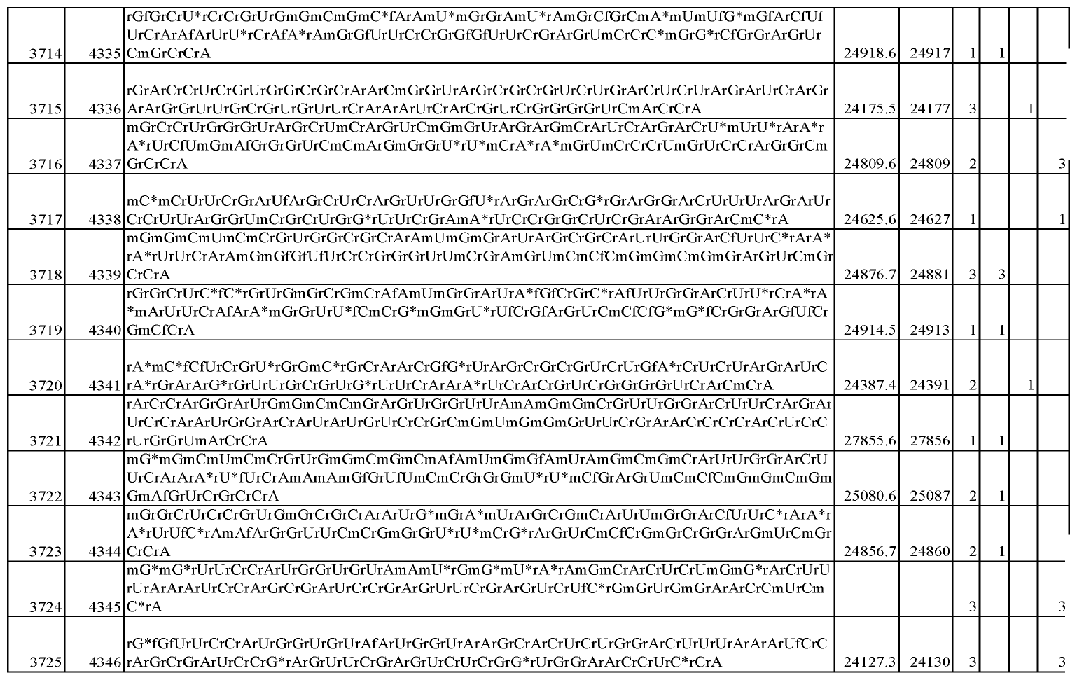

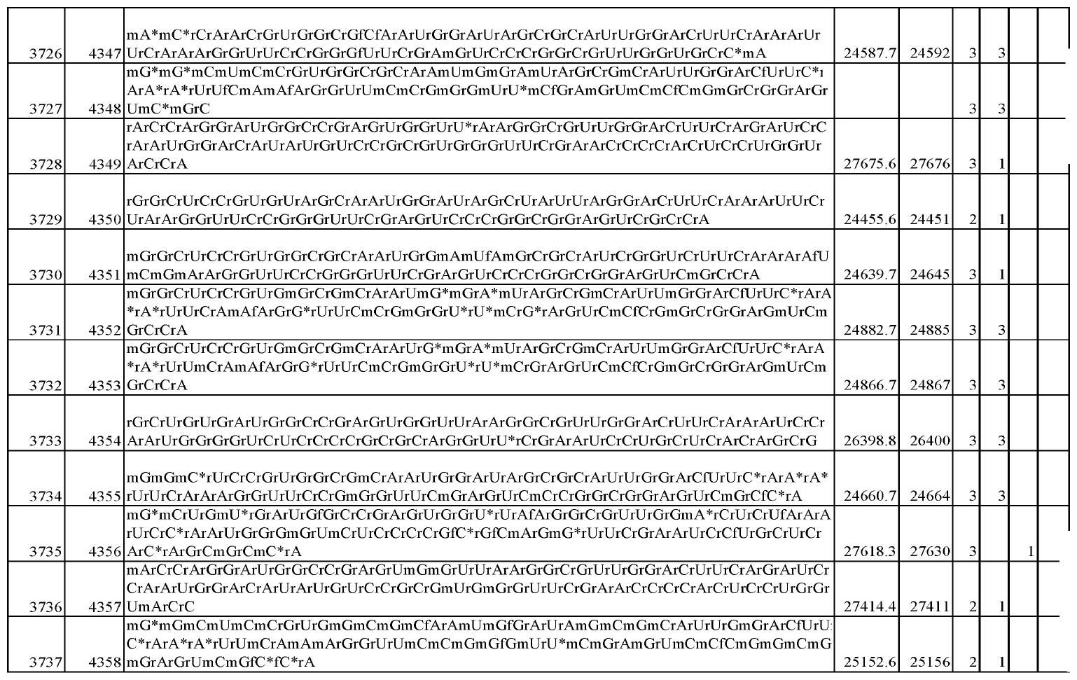

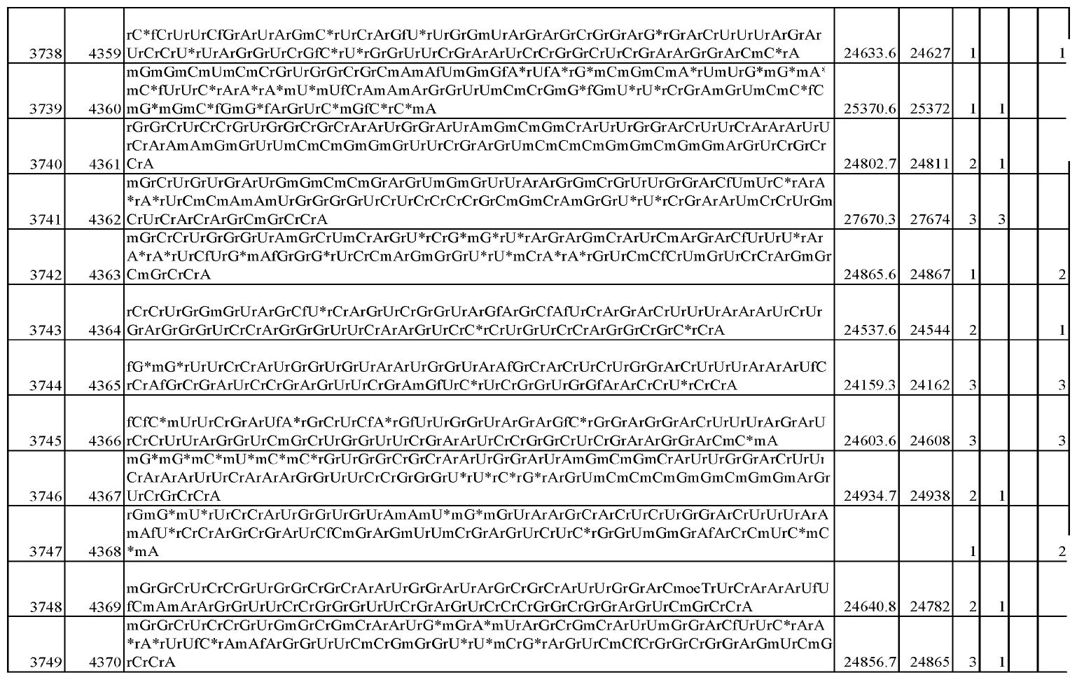

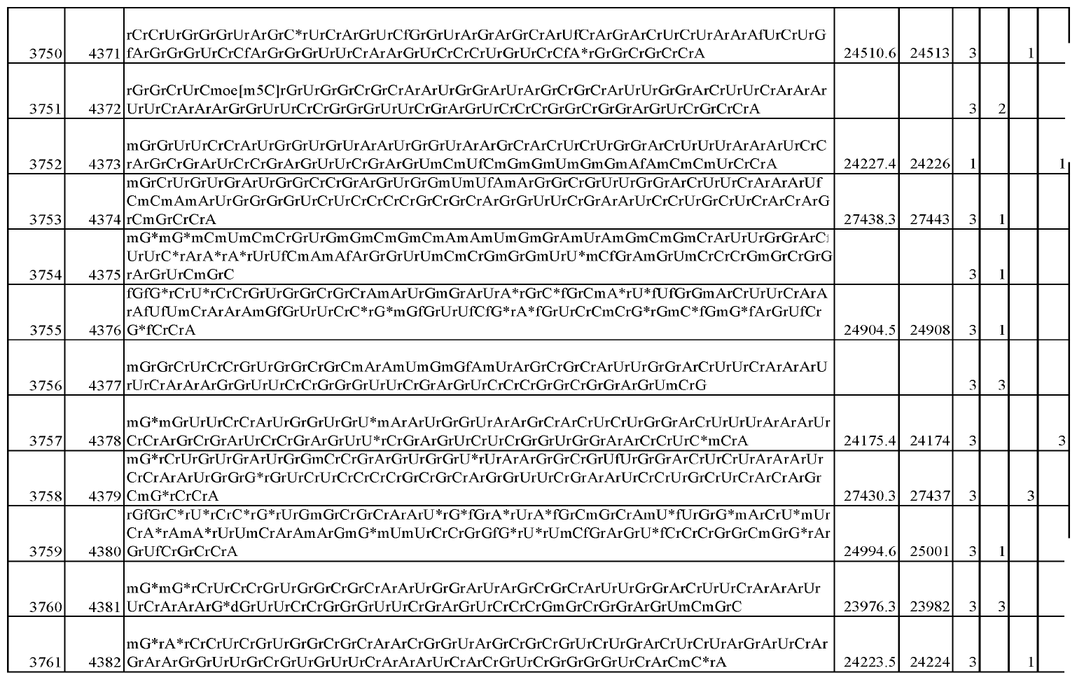

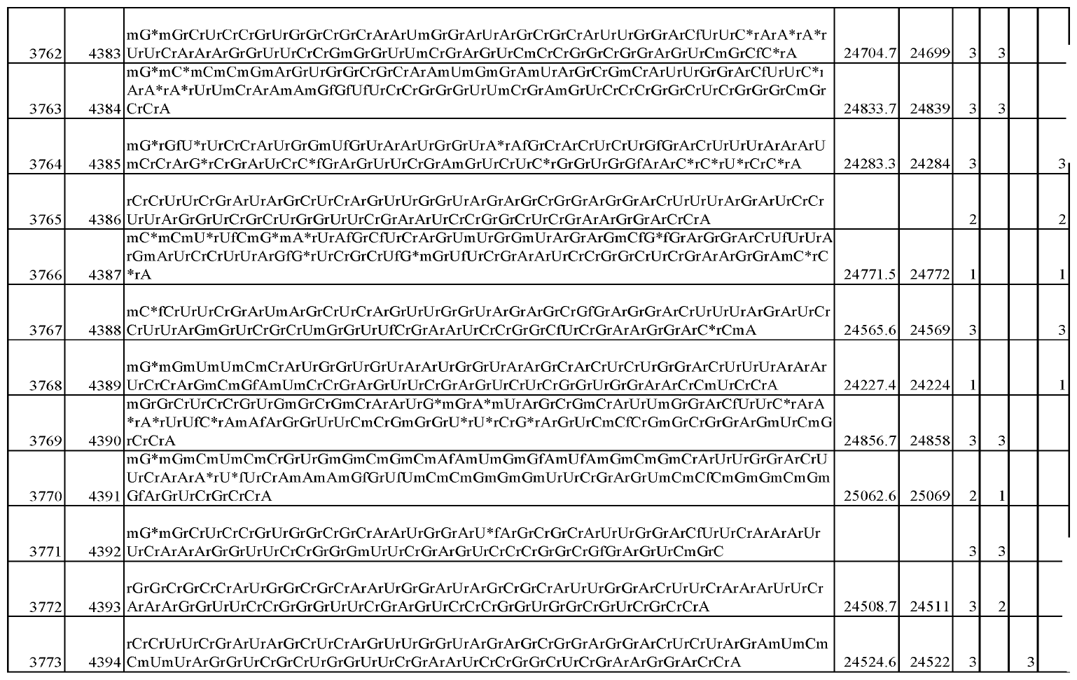

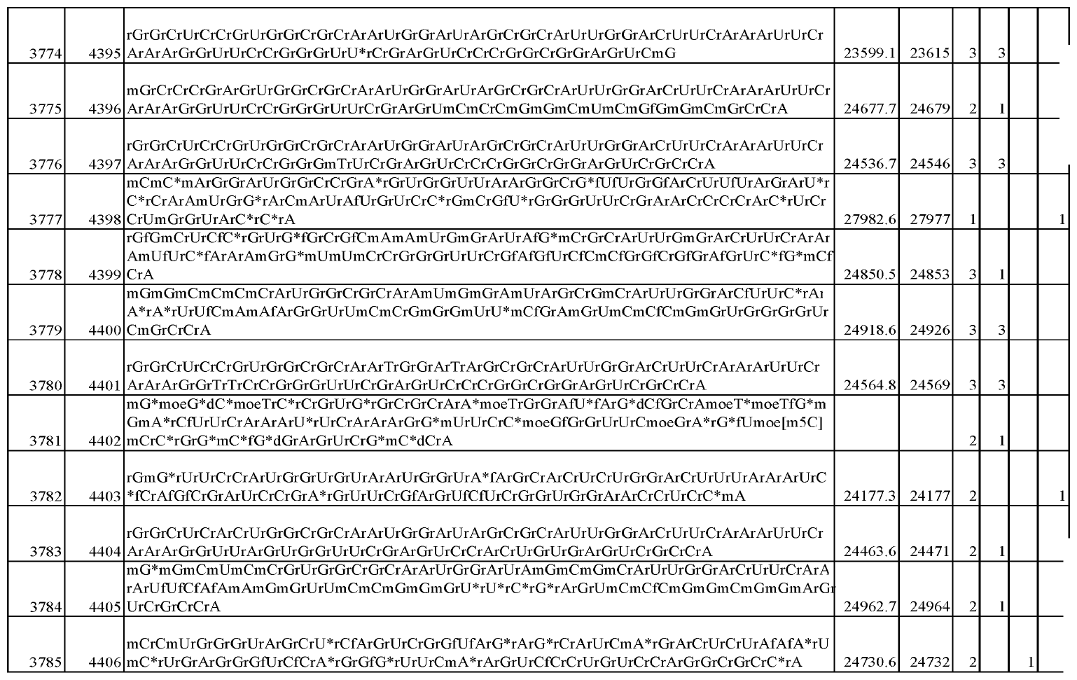

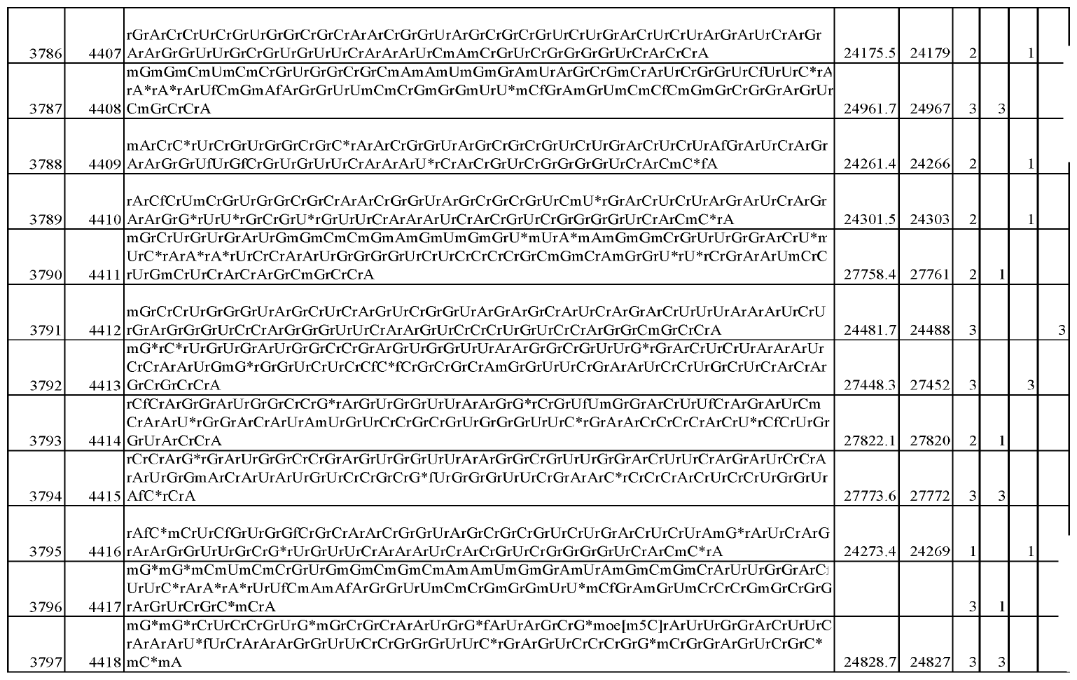

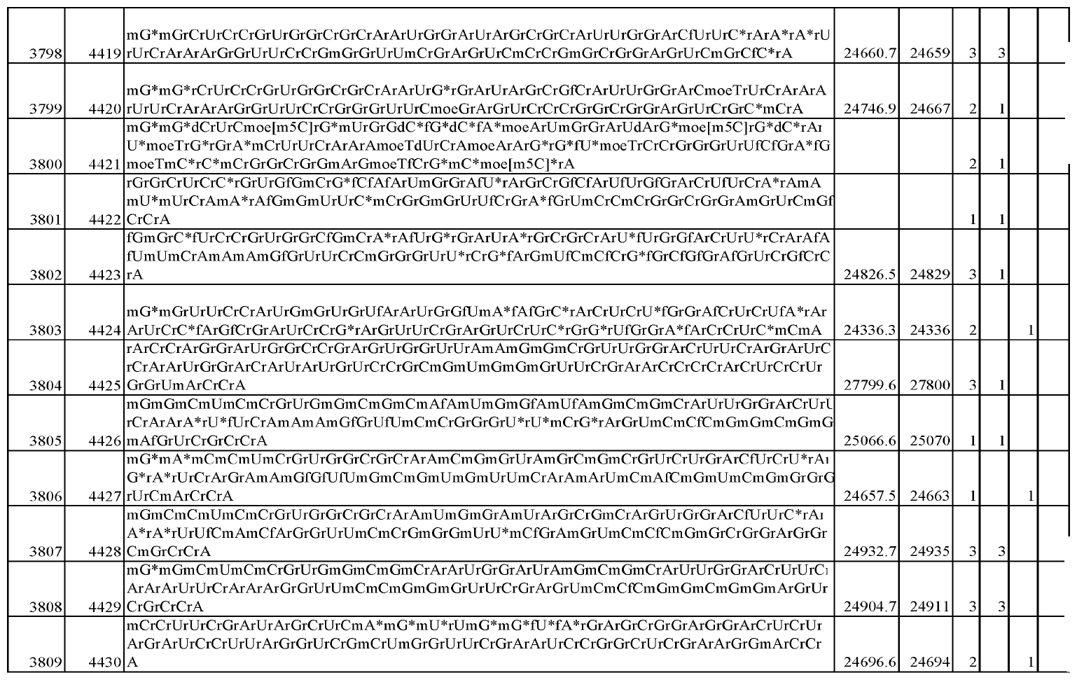

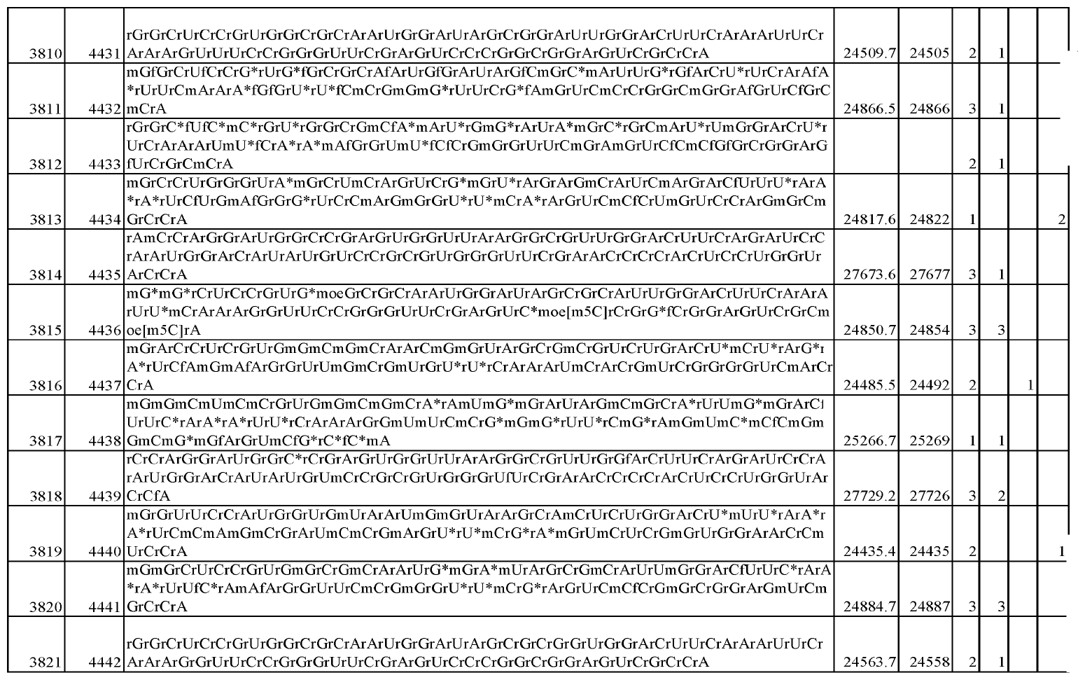

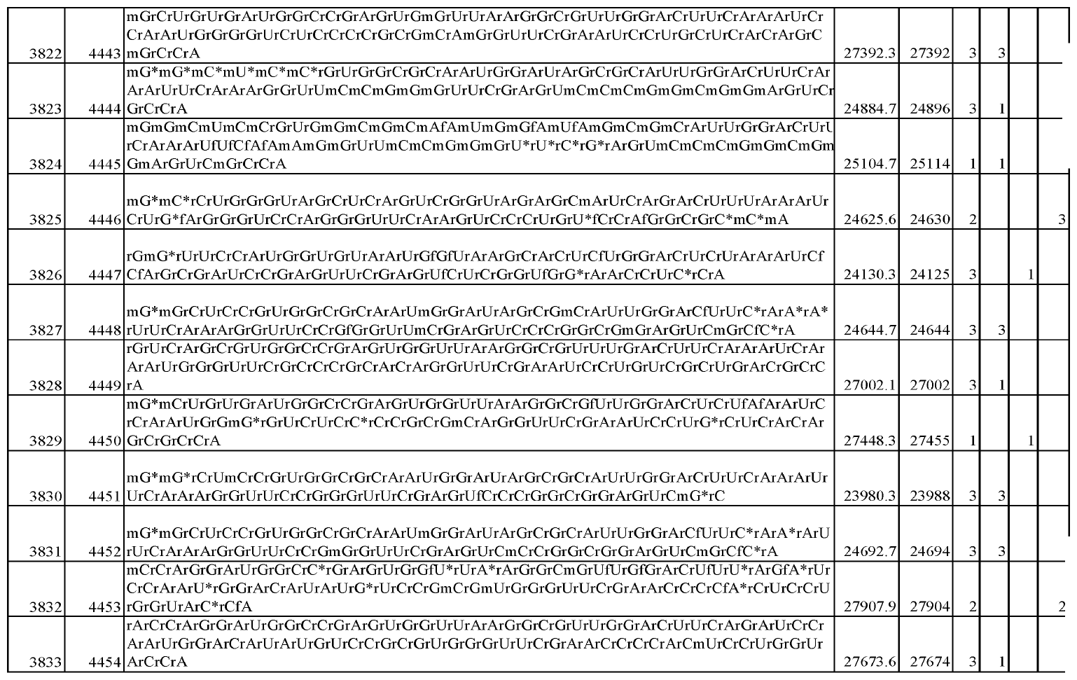

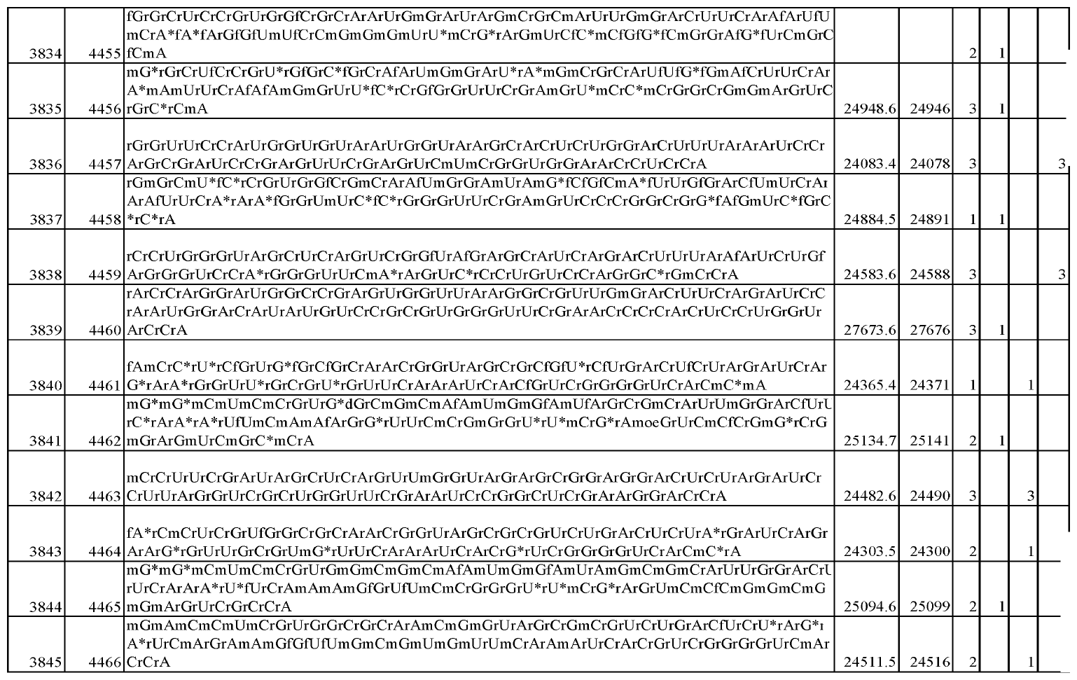

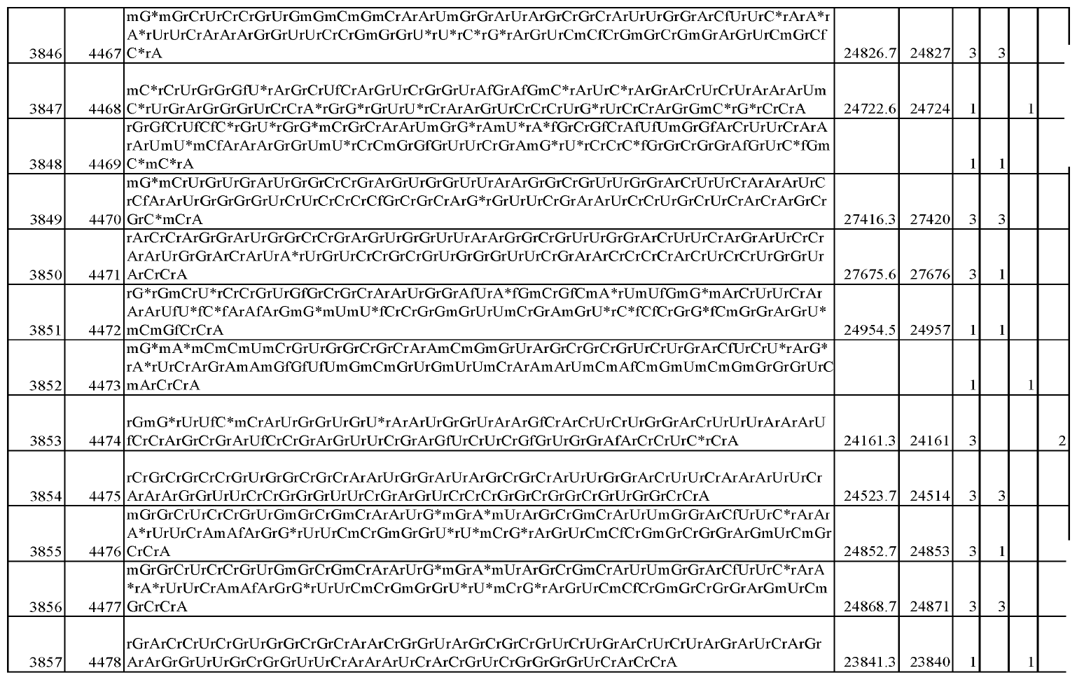

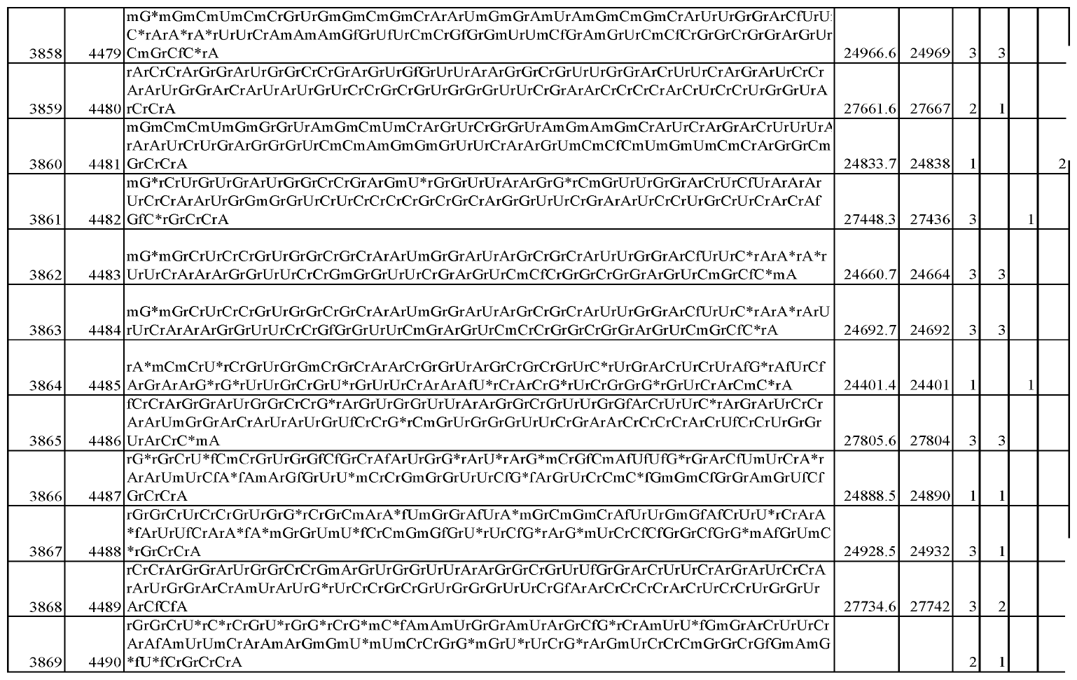

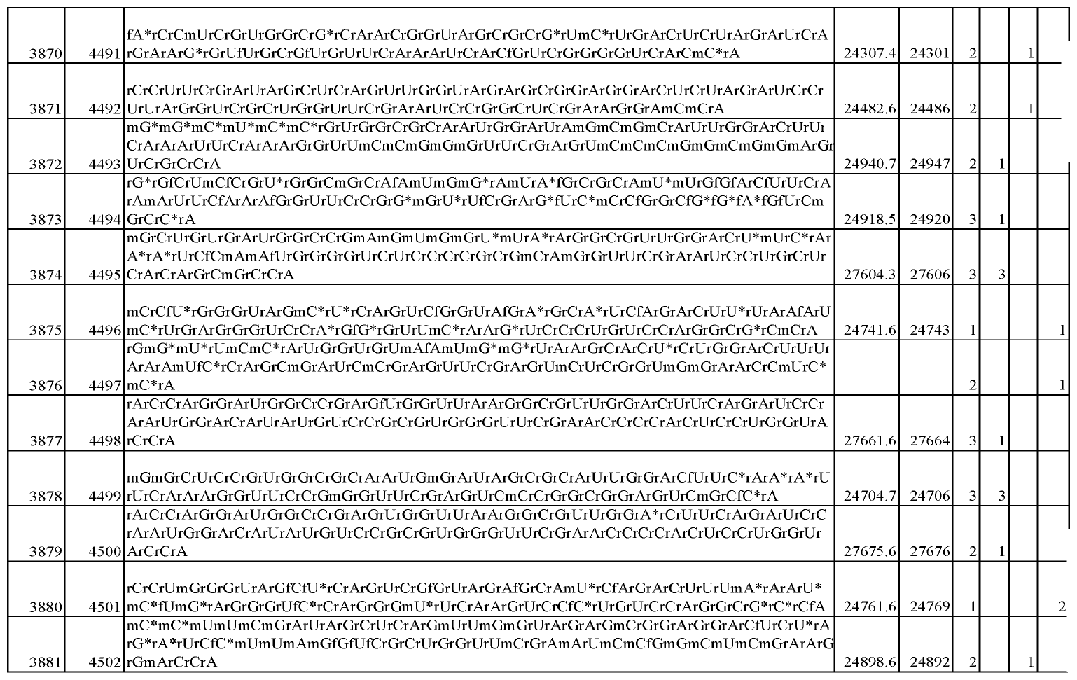

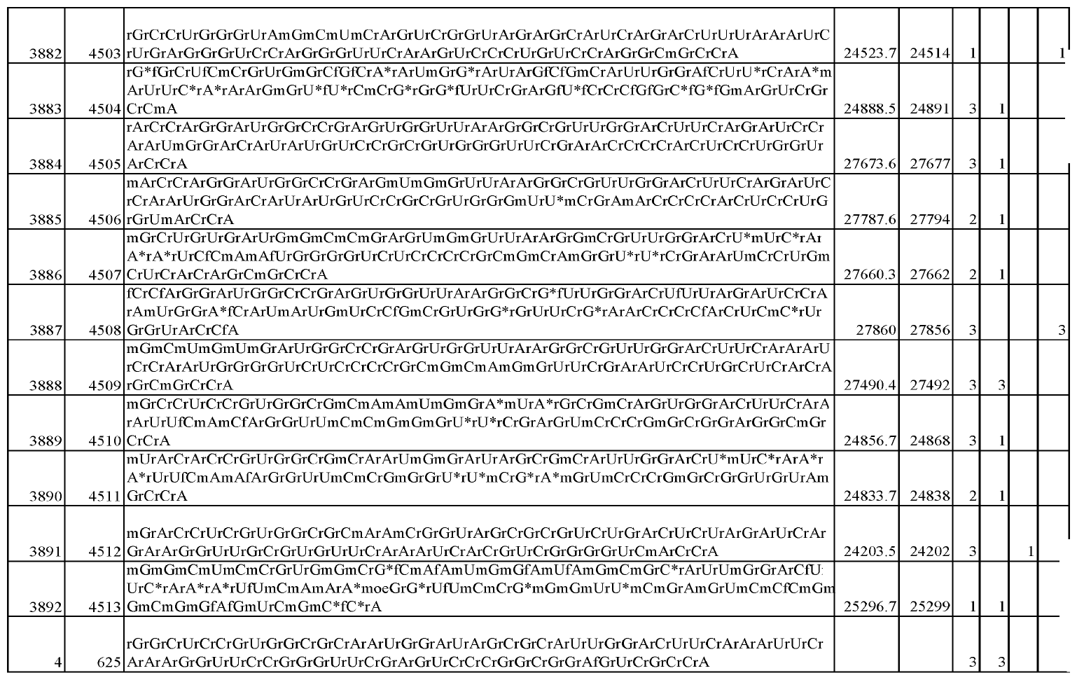

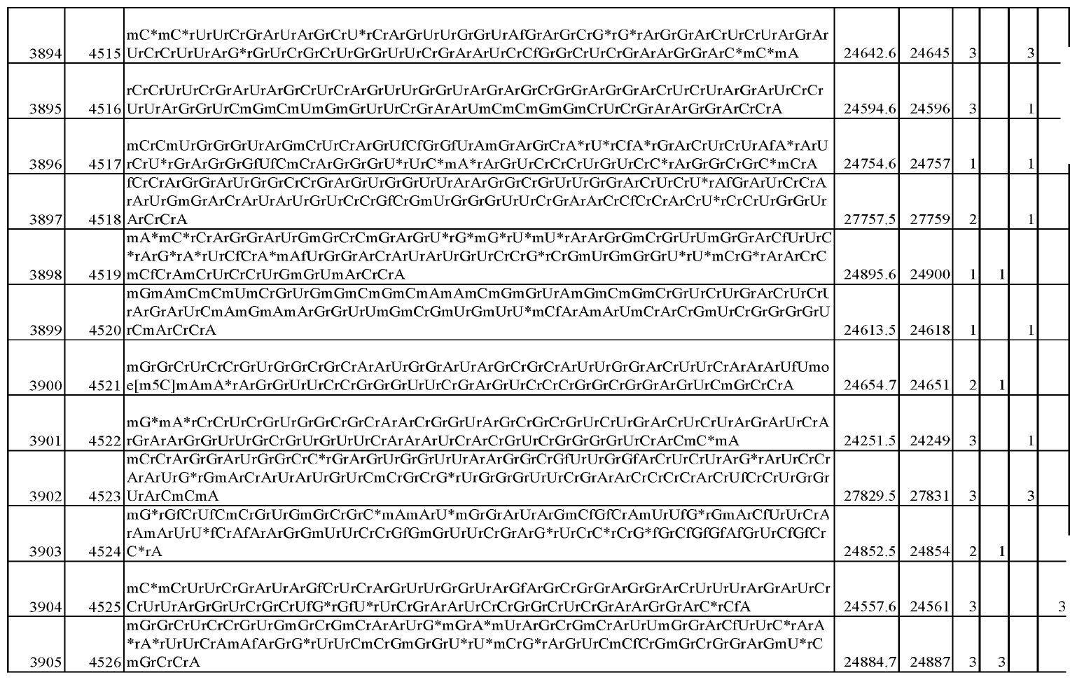

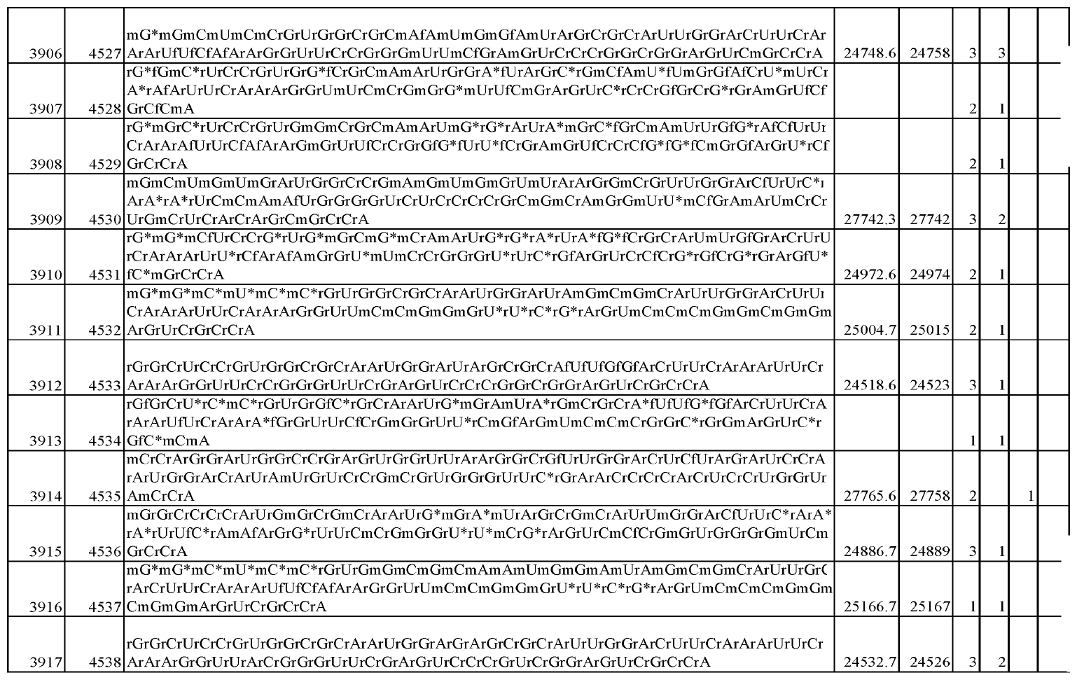

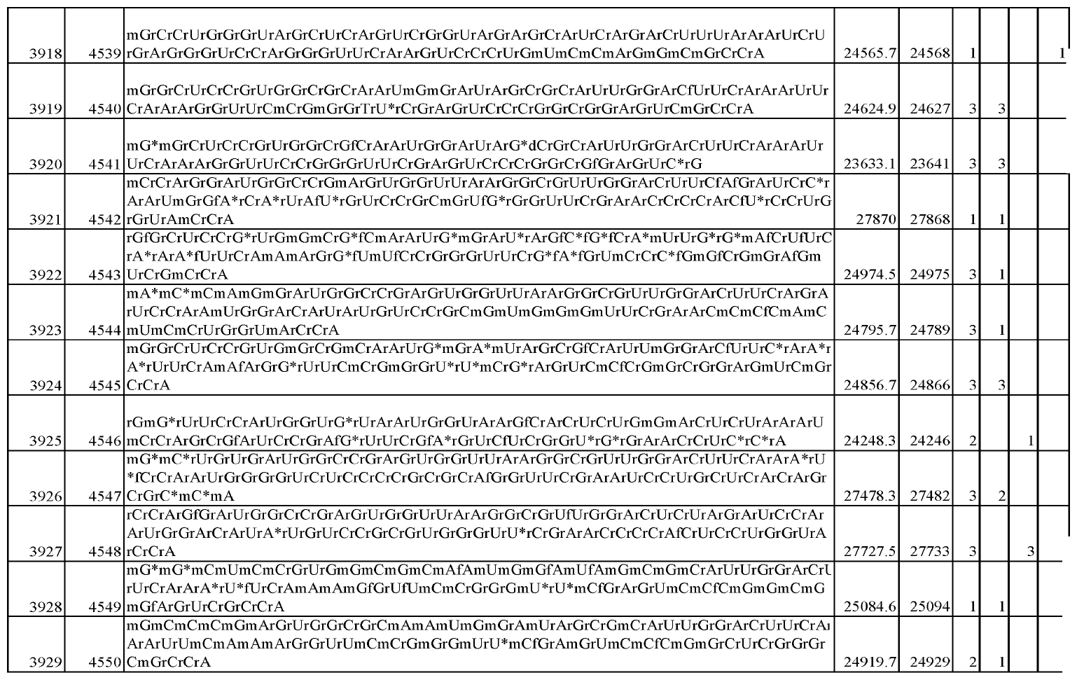

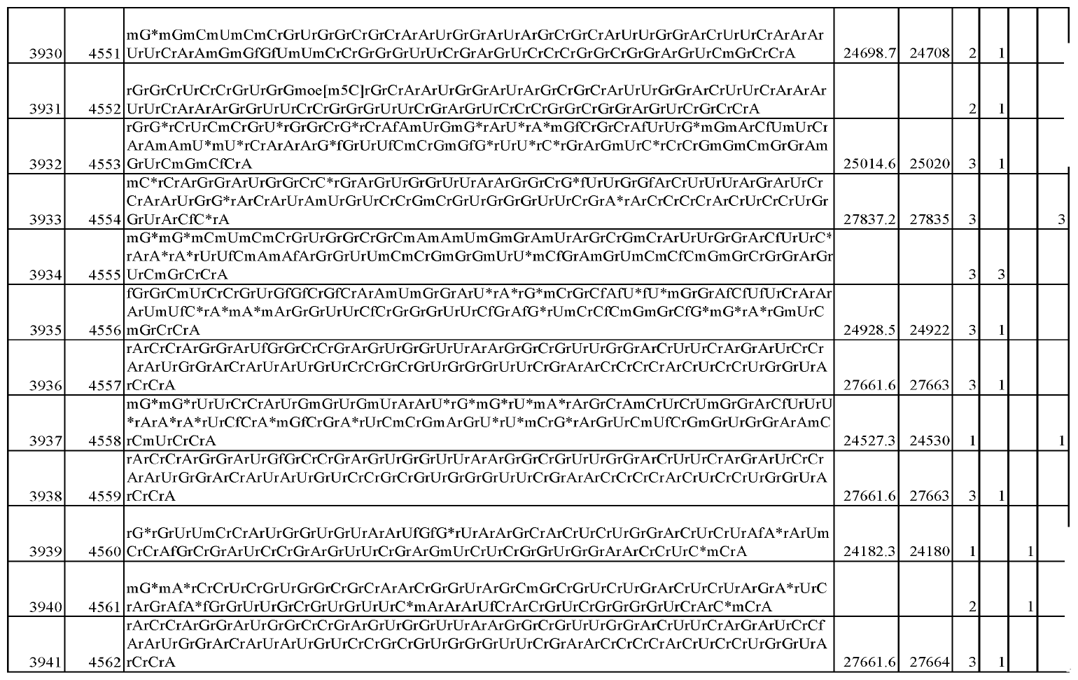

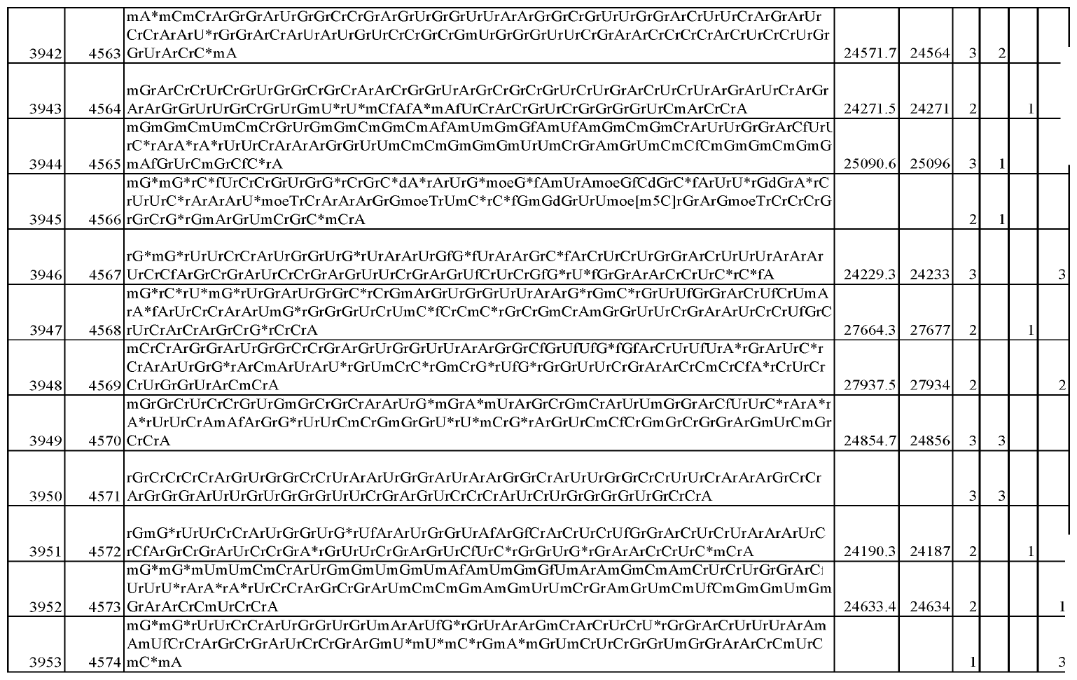

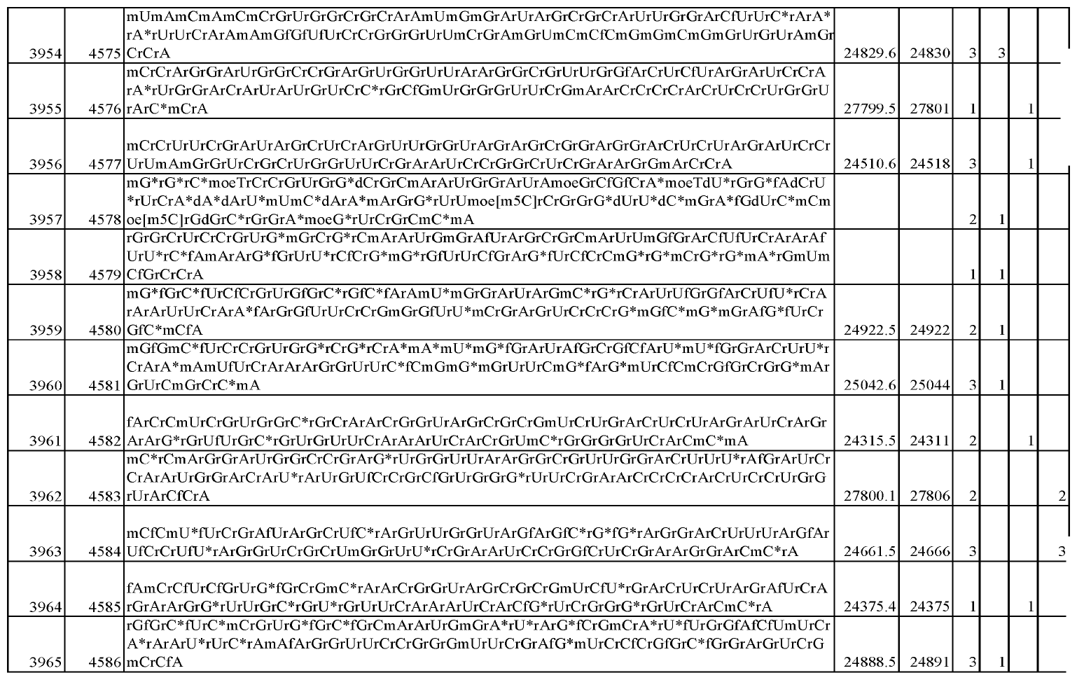

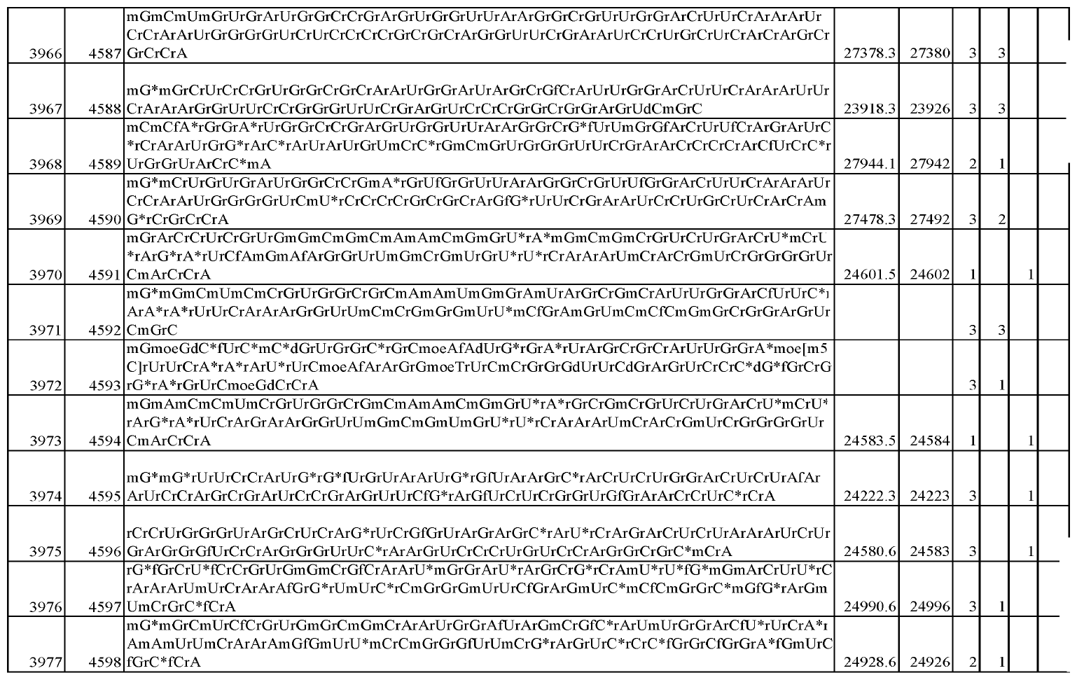

This example describes administration of exemplary TREMs to Fabry-patient derived fibroblasts (GLA R220X) to assess rescue of full-length GLA protein expression and activity. Fabry patient fibroblasts (Coriell, GM00881; GLAR220X) and normal healthy fibroblasts (Coriell, GM03377) were transfected with 40 nM of either a chemically unmodified TREM (TREM NO. 2309) or an exemplary TREM comprising non-naturally occurring modifications (TREM NO. 78). GLA protein rescue was assessed by Western blotting 48 hours post-transfection in both cell lines. As shown in FIG. 4, administration of both TREMs induced rescue of full-length GLA protein, with the chemically modified TREM NO. 78 affording a considerable increase in the total GLA produced over the chemically unmodified TREM NO. 2309.

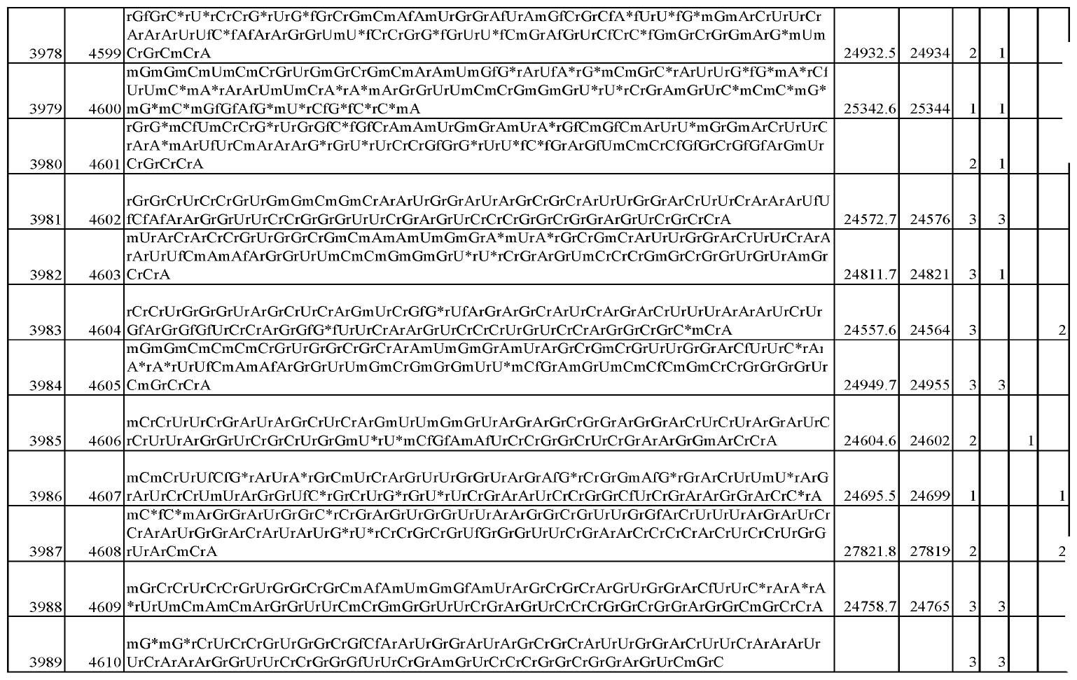

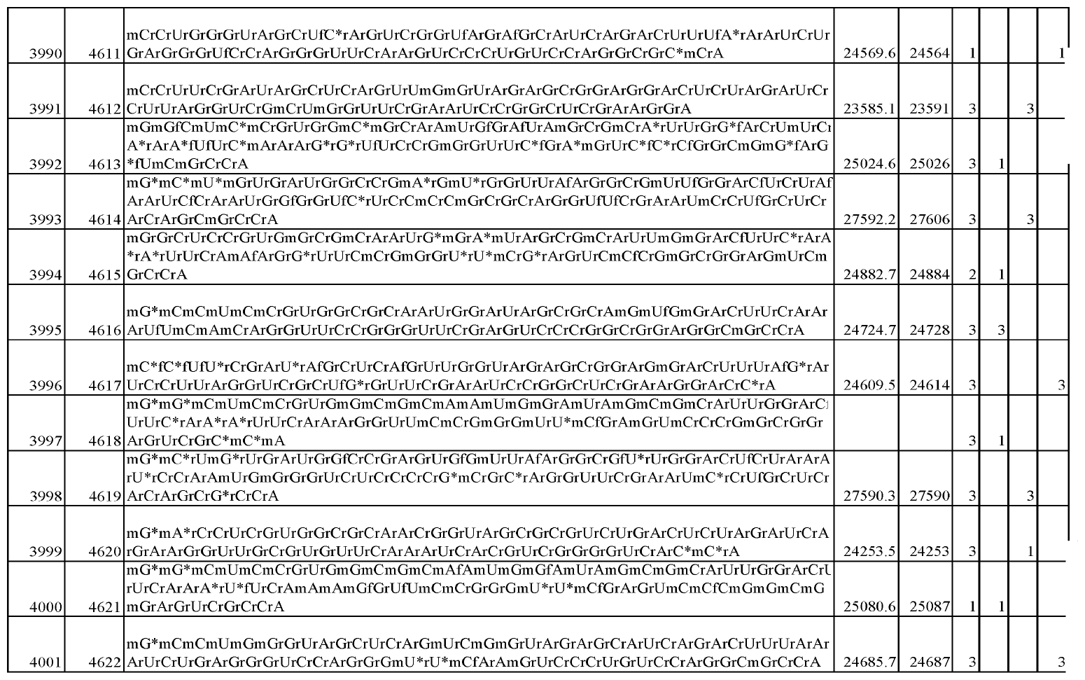

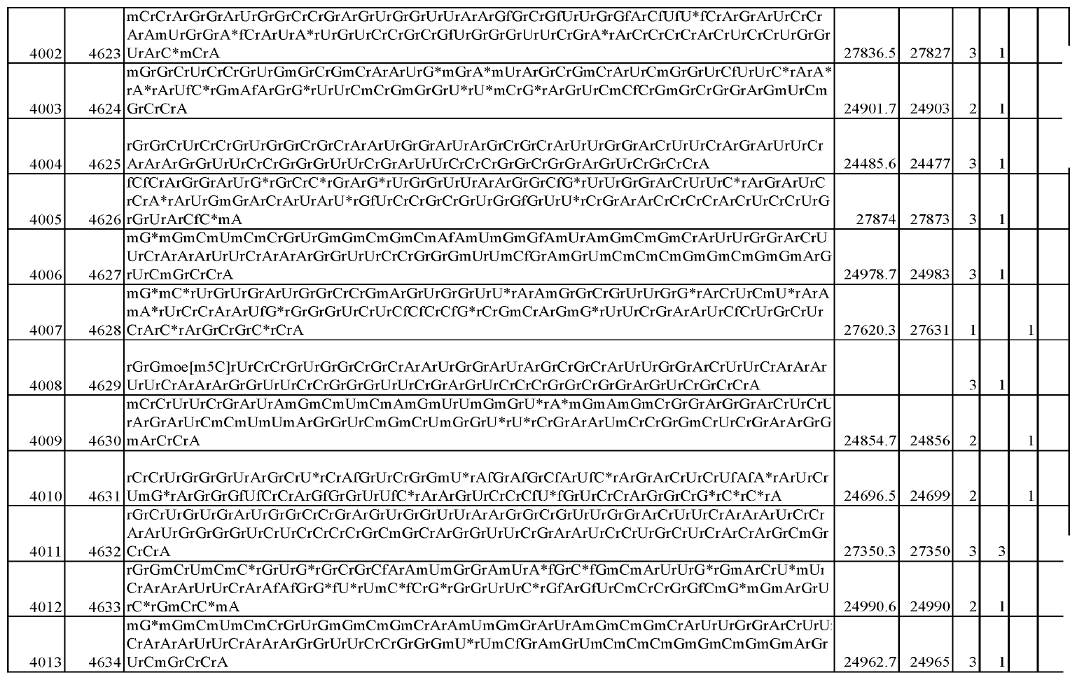

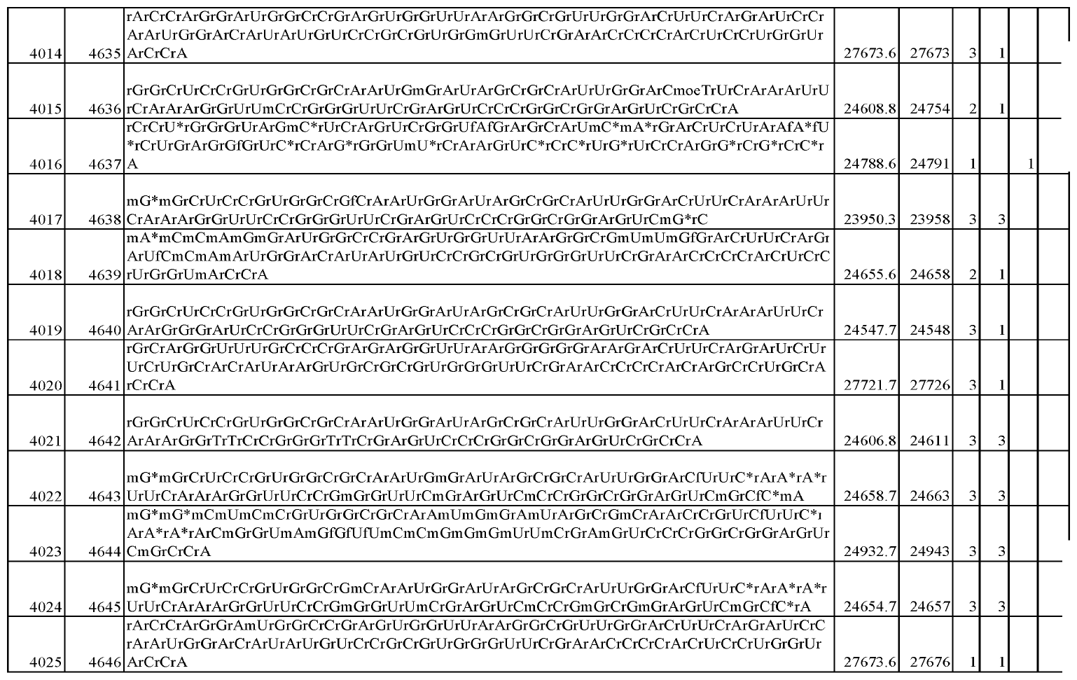

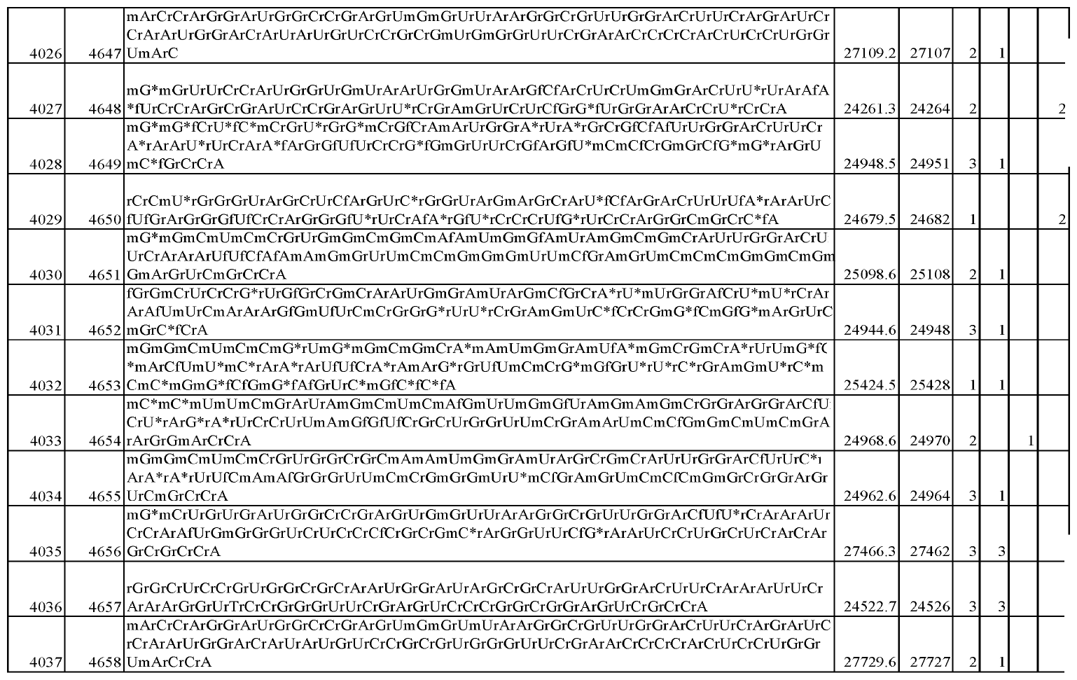

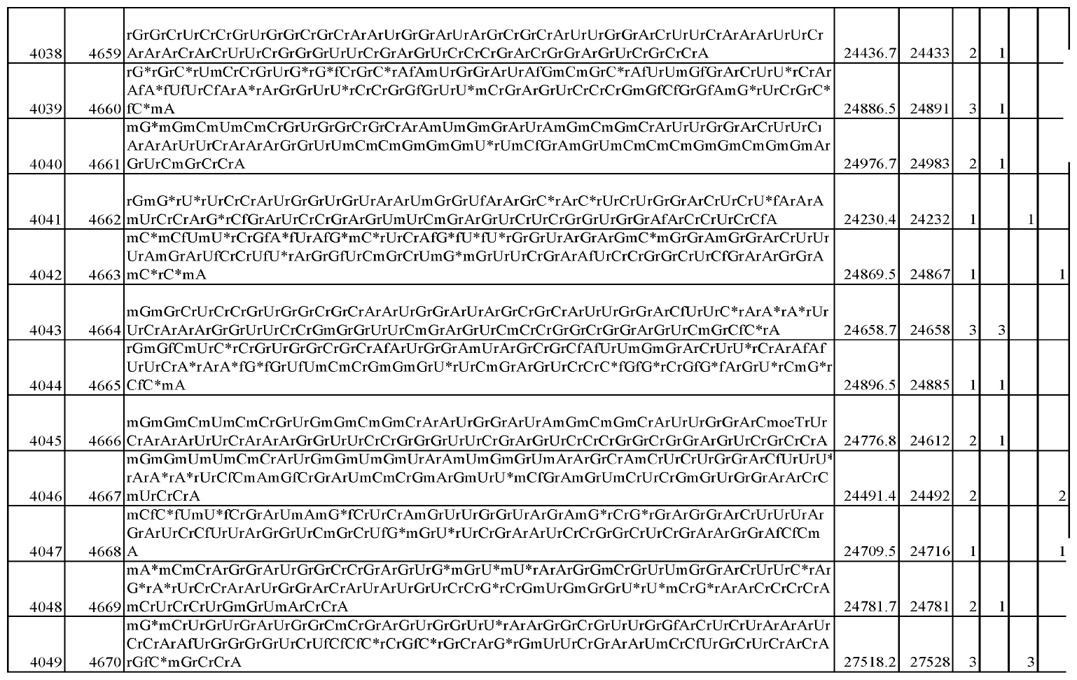

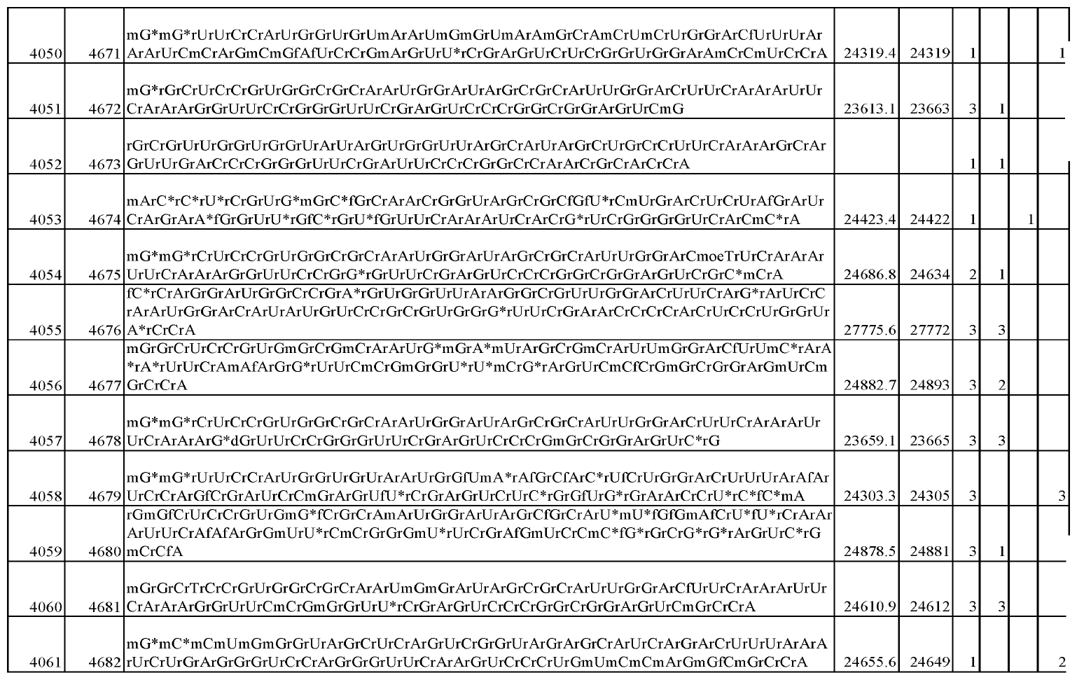

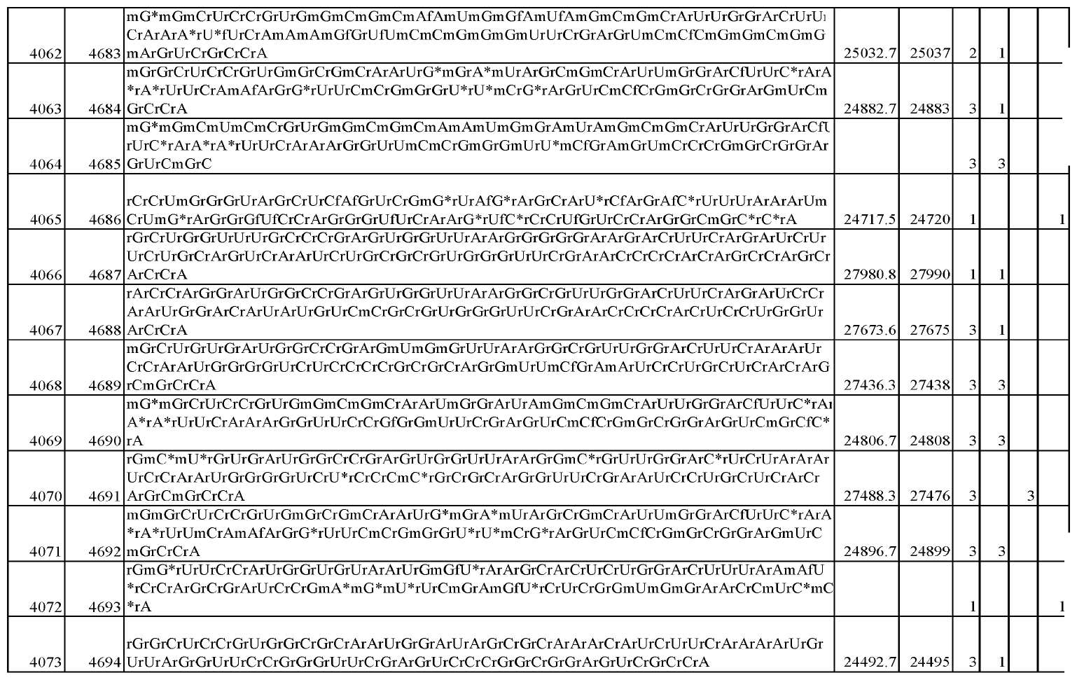

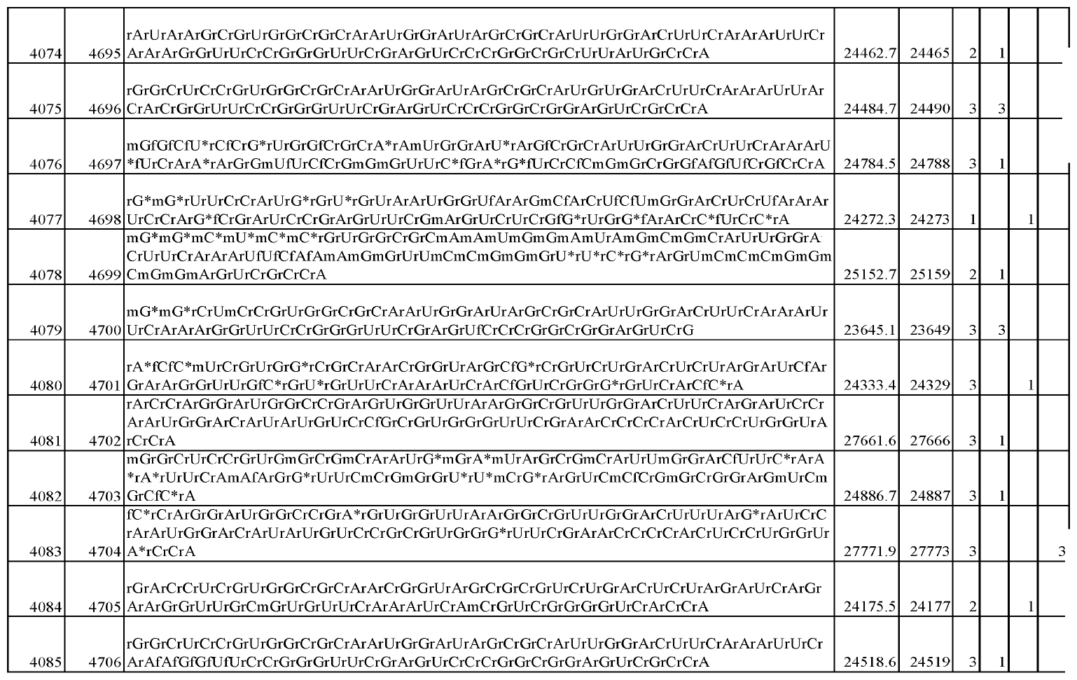

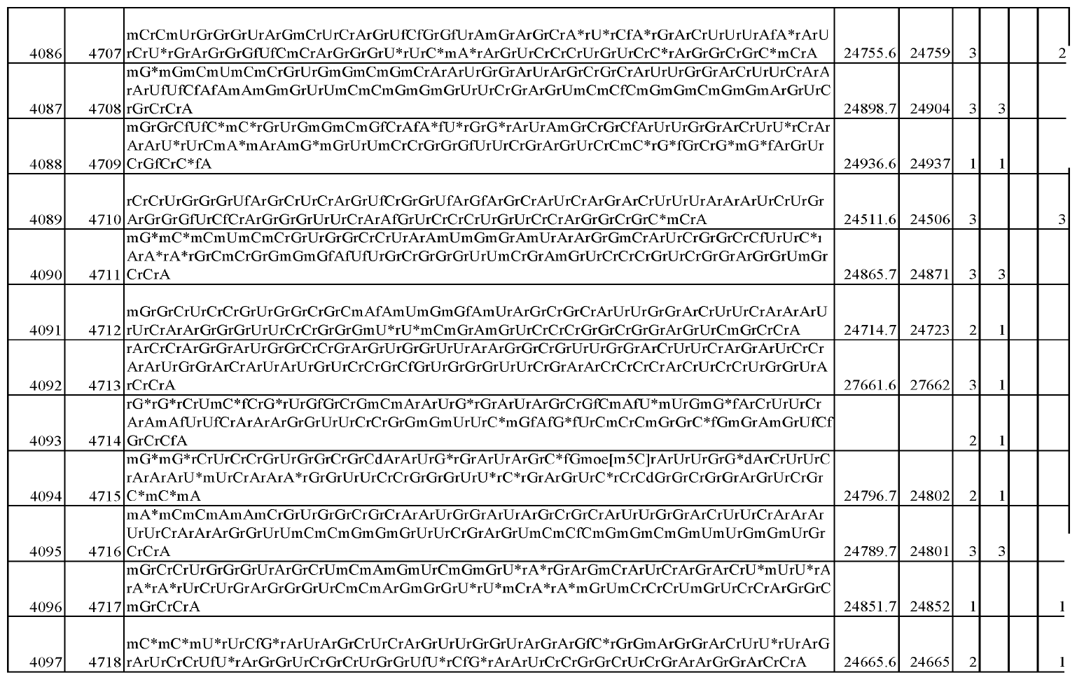

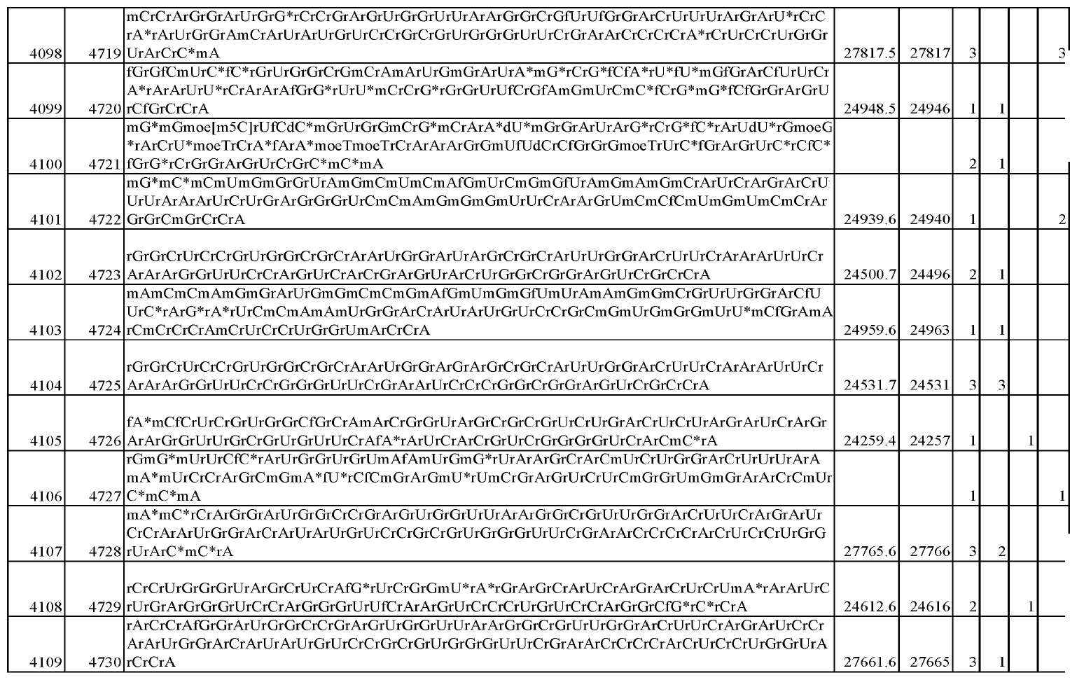

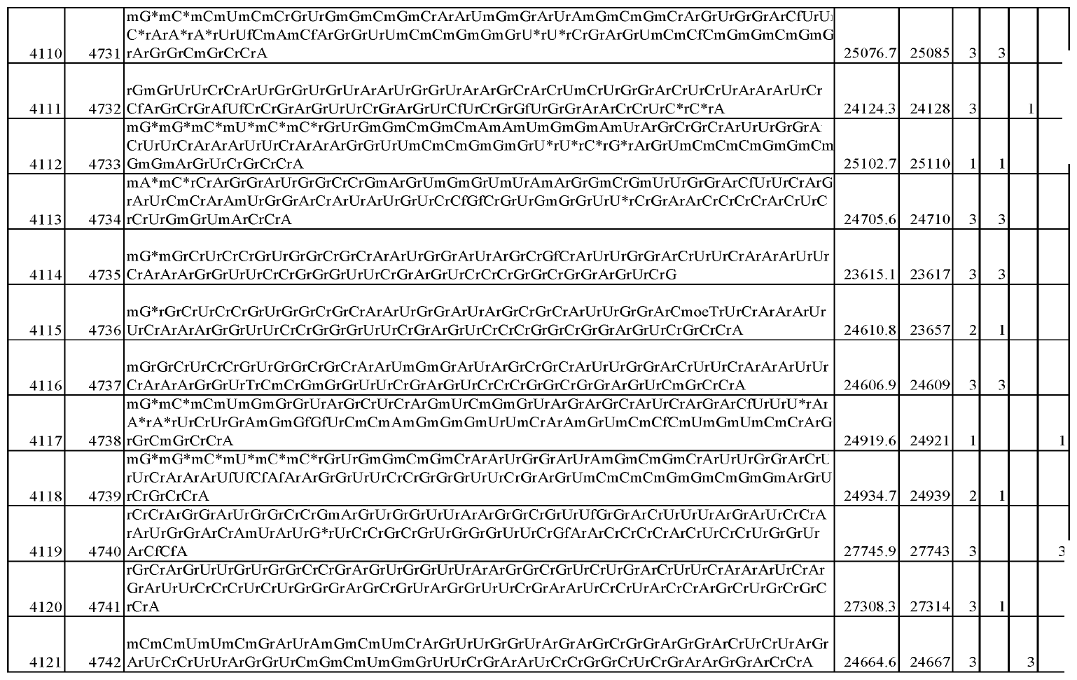

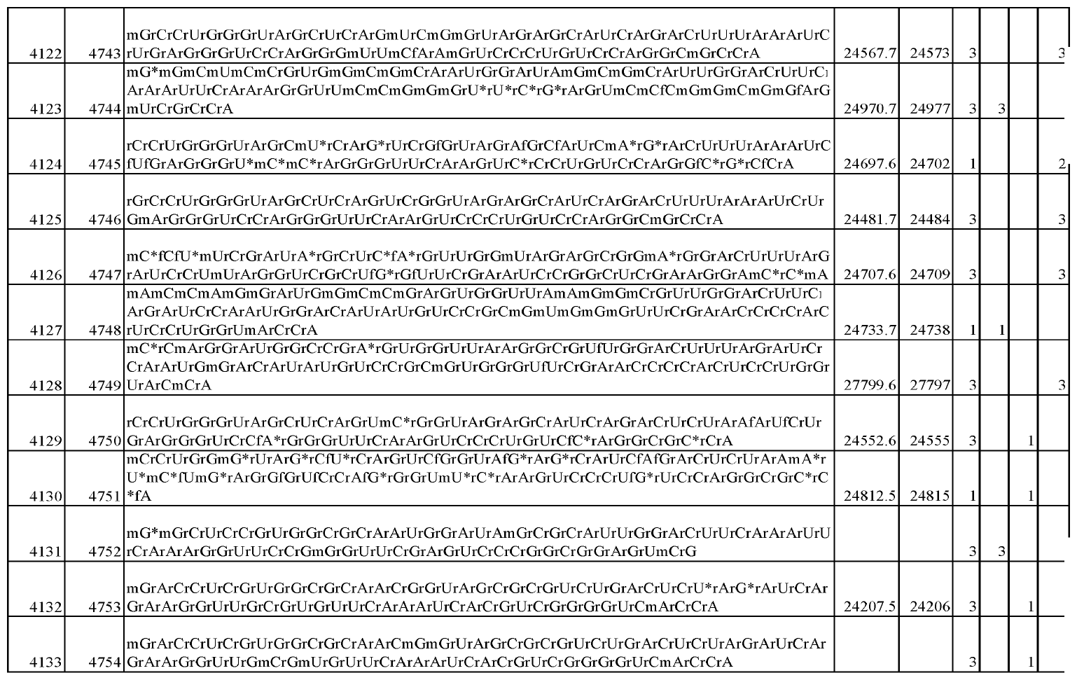

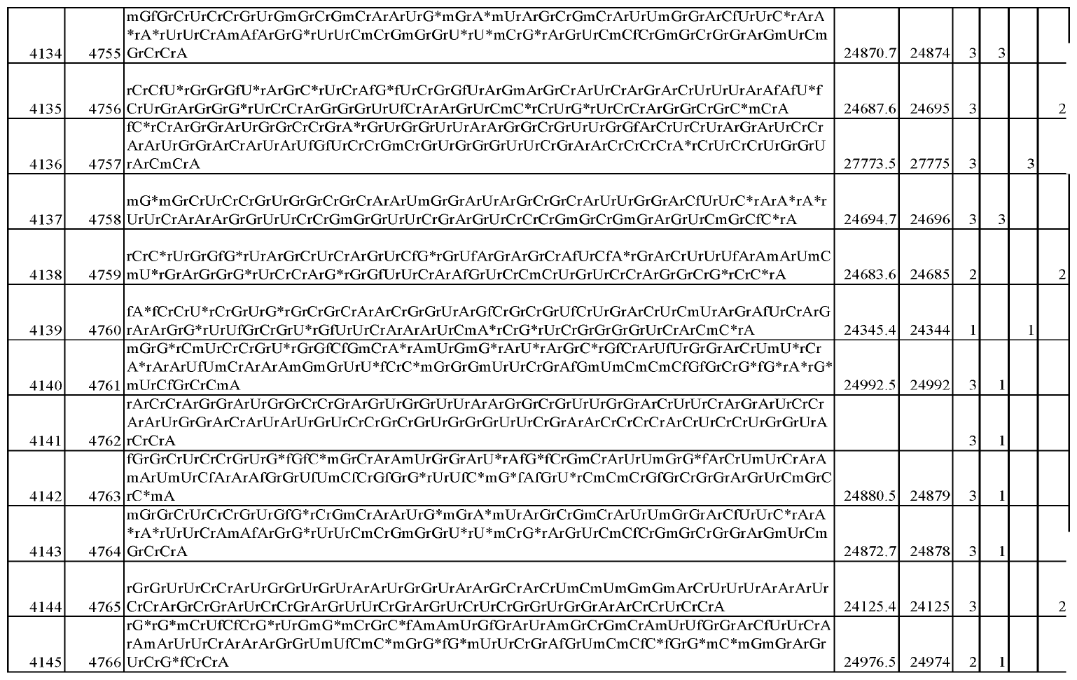

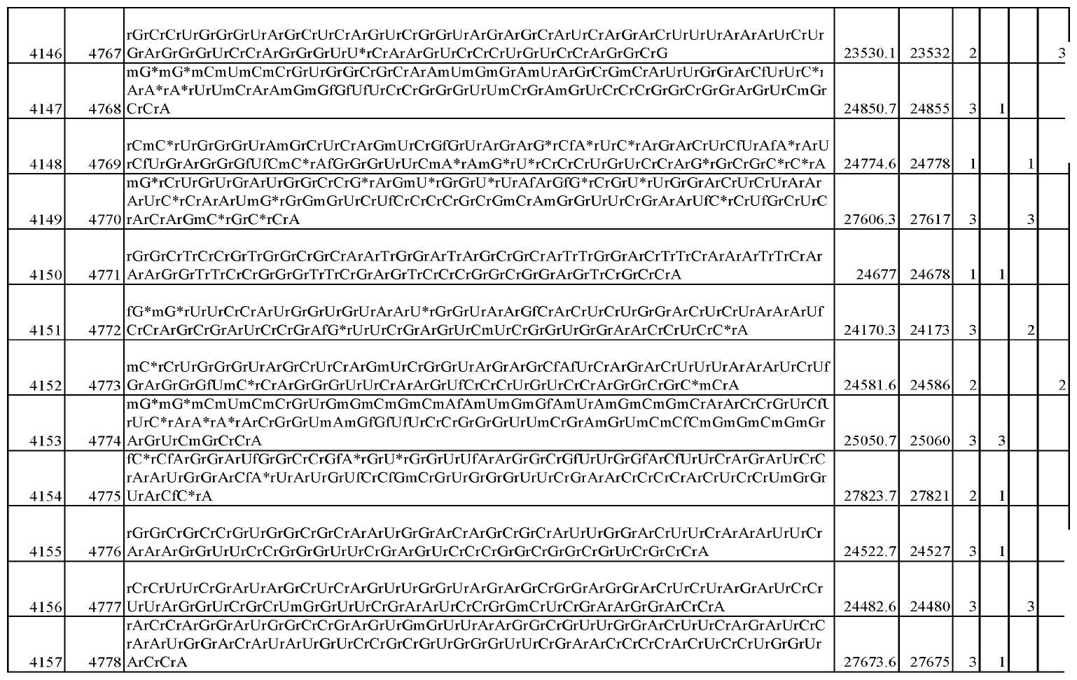

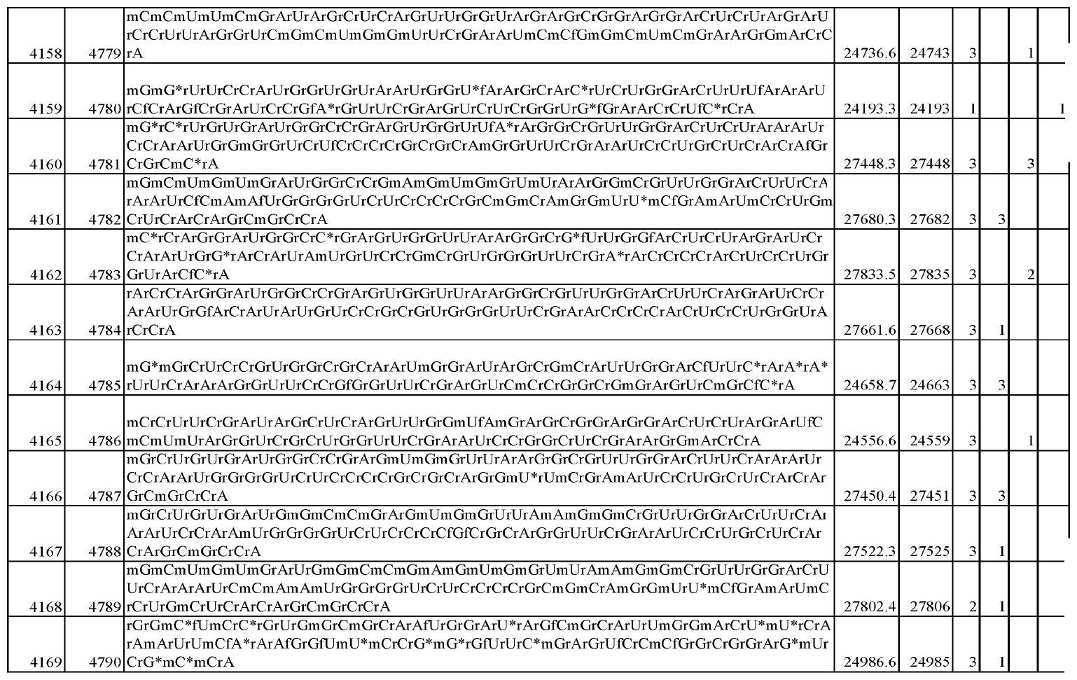

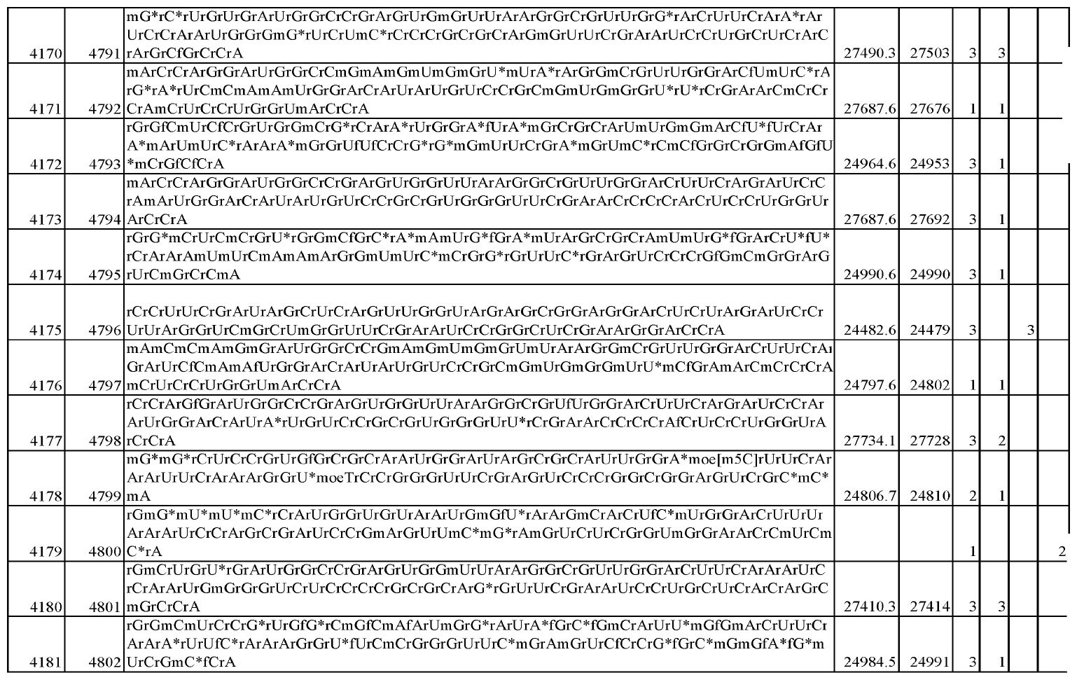

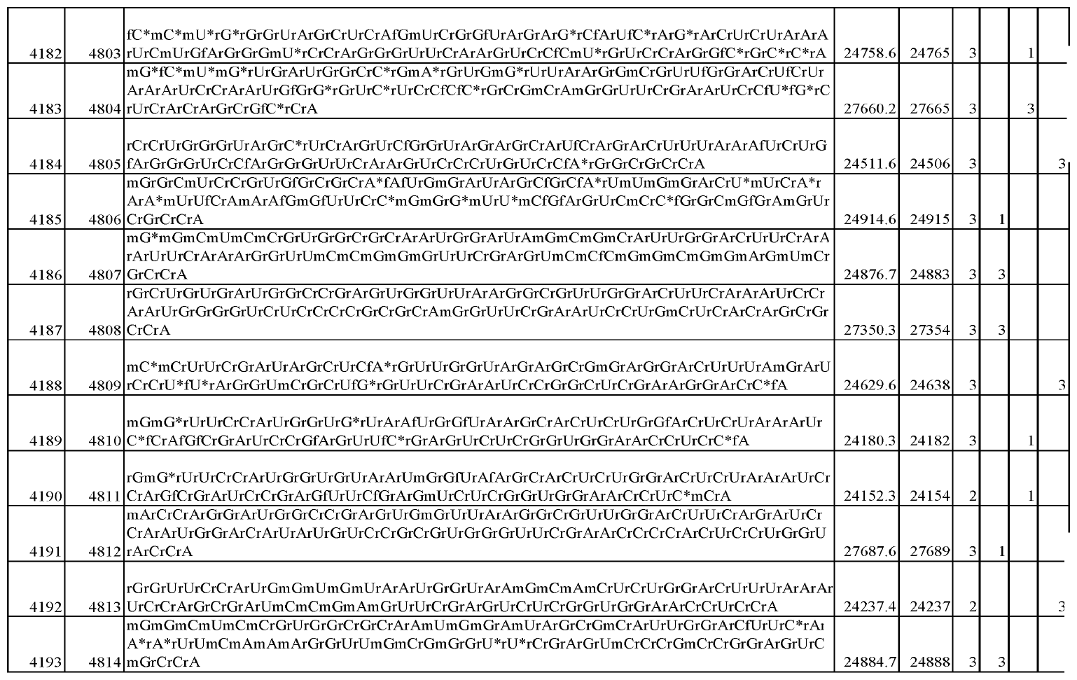

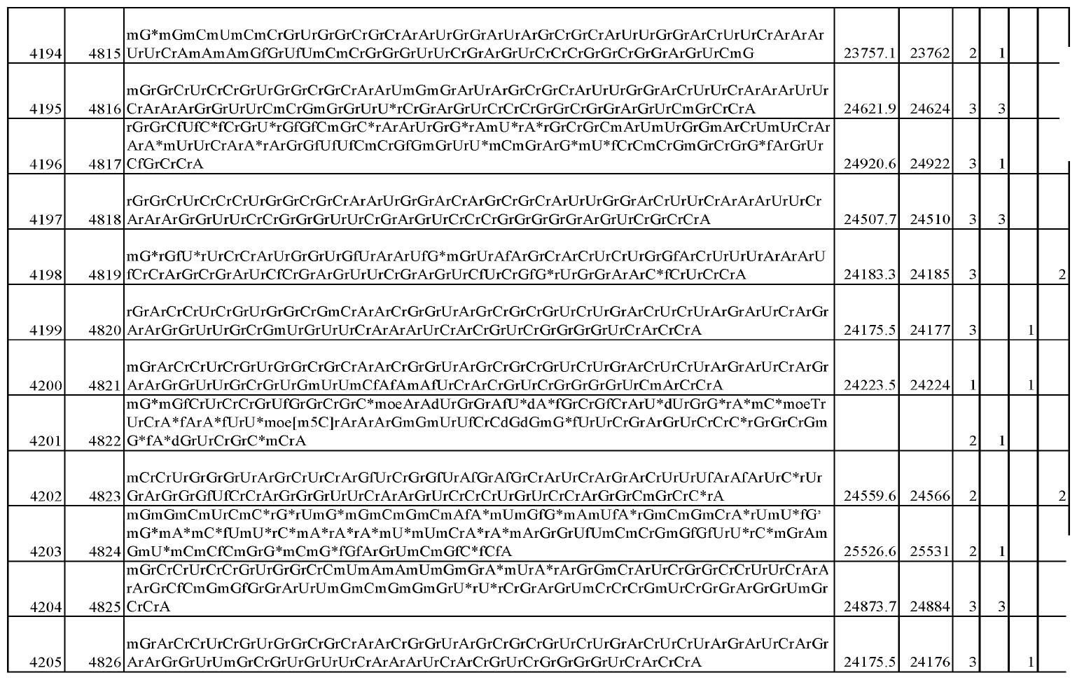

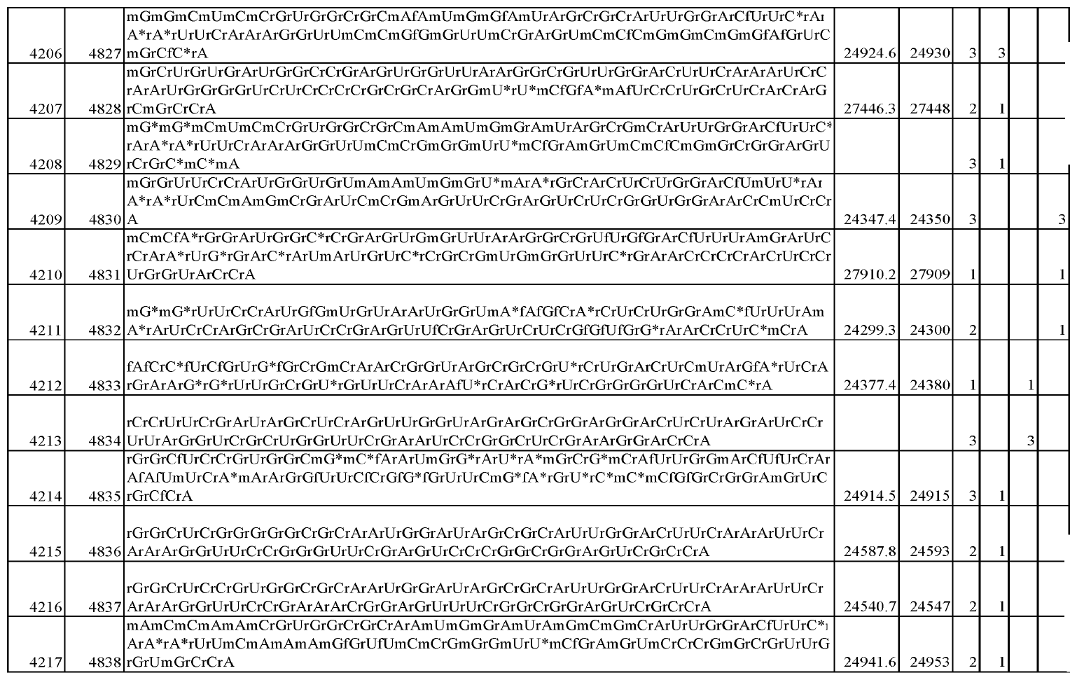

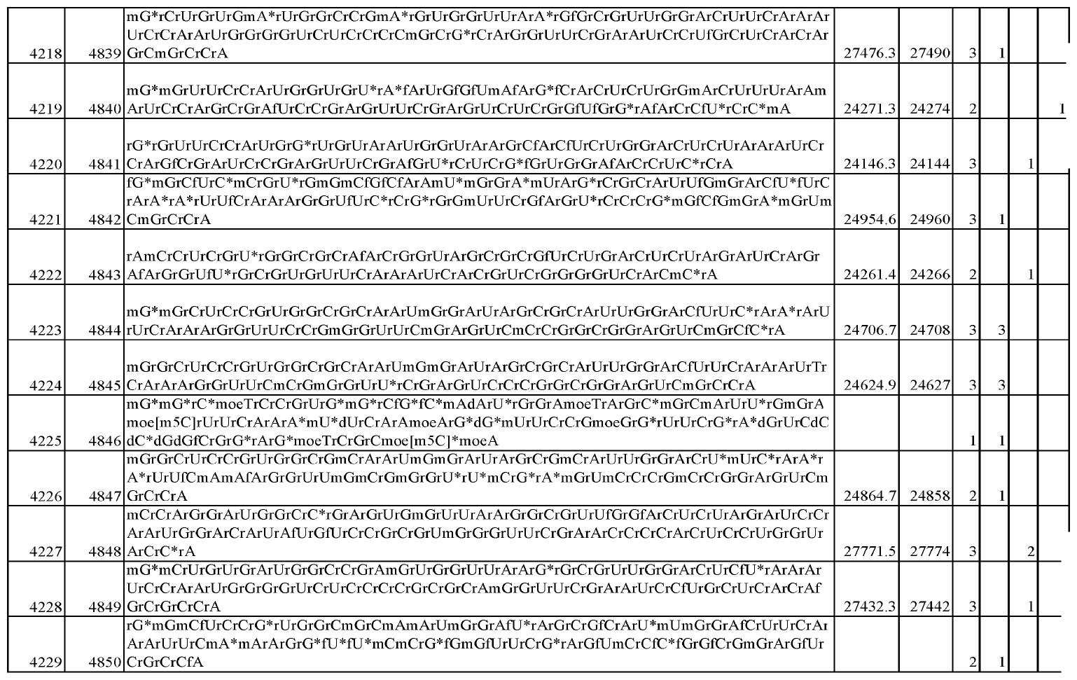

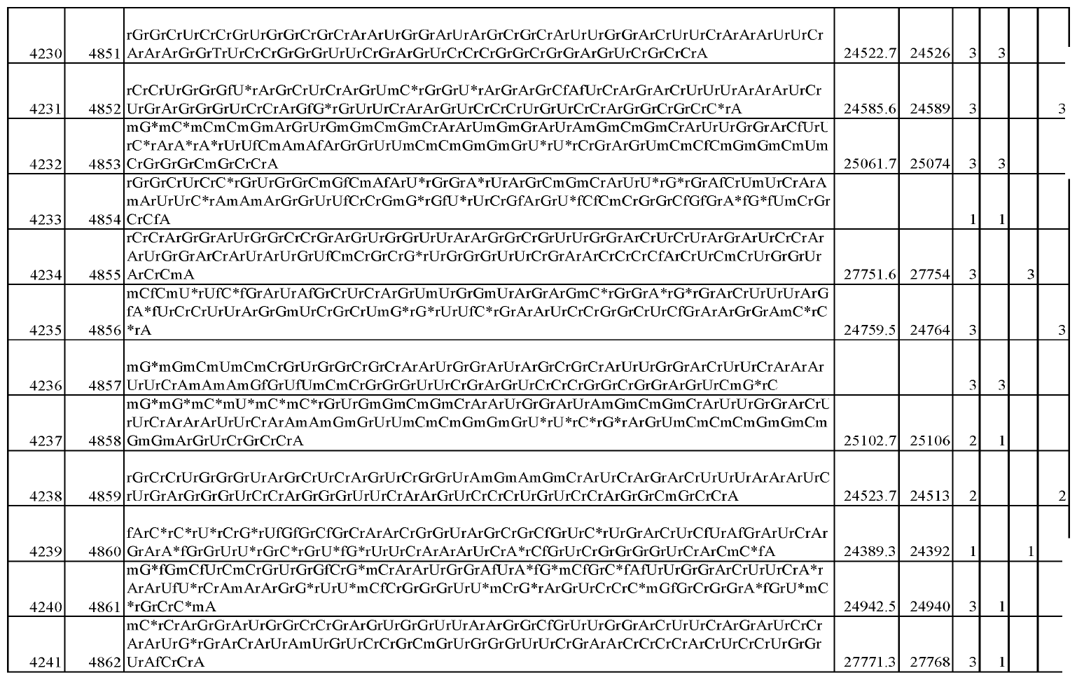

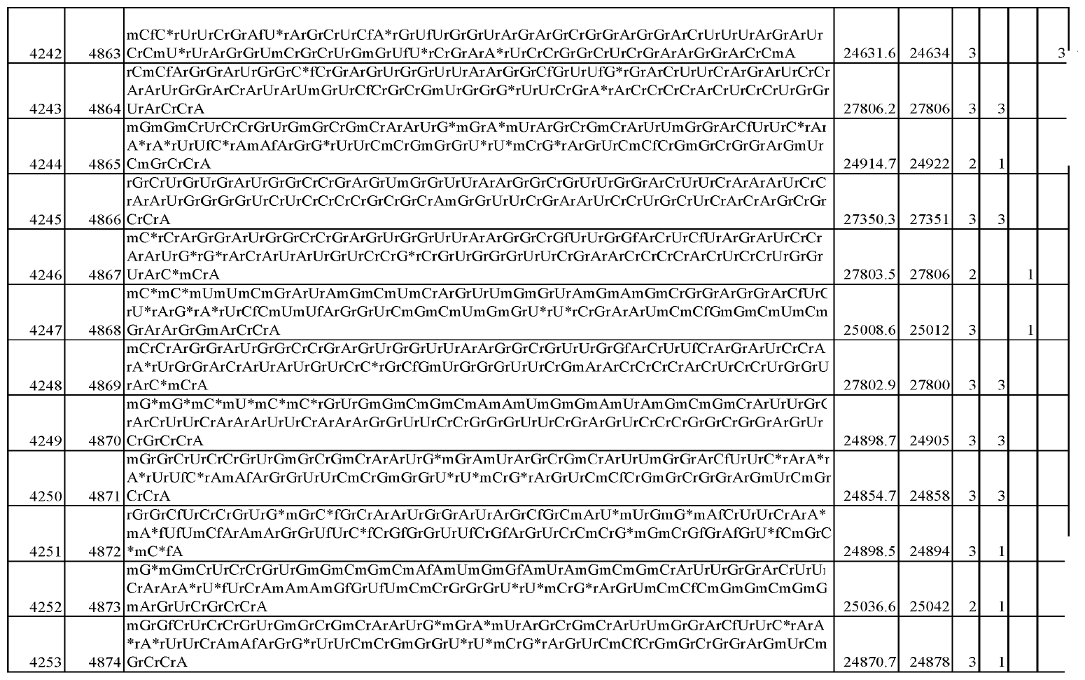

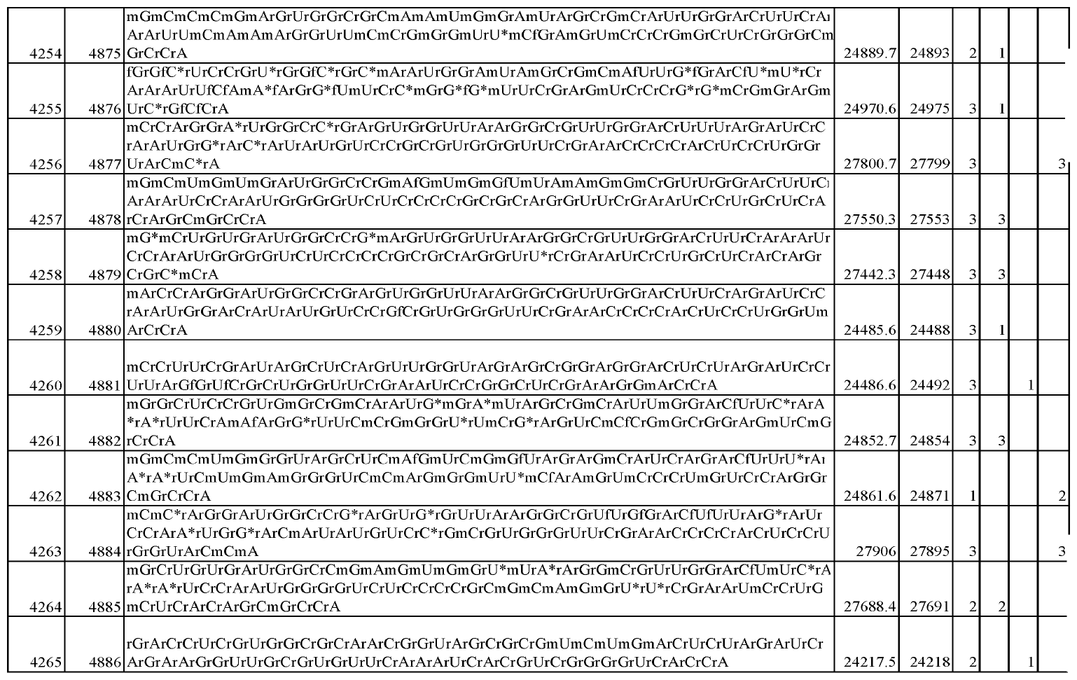

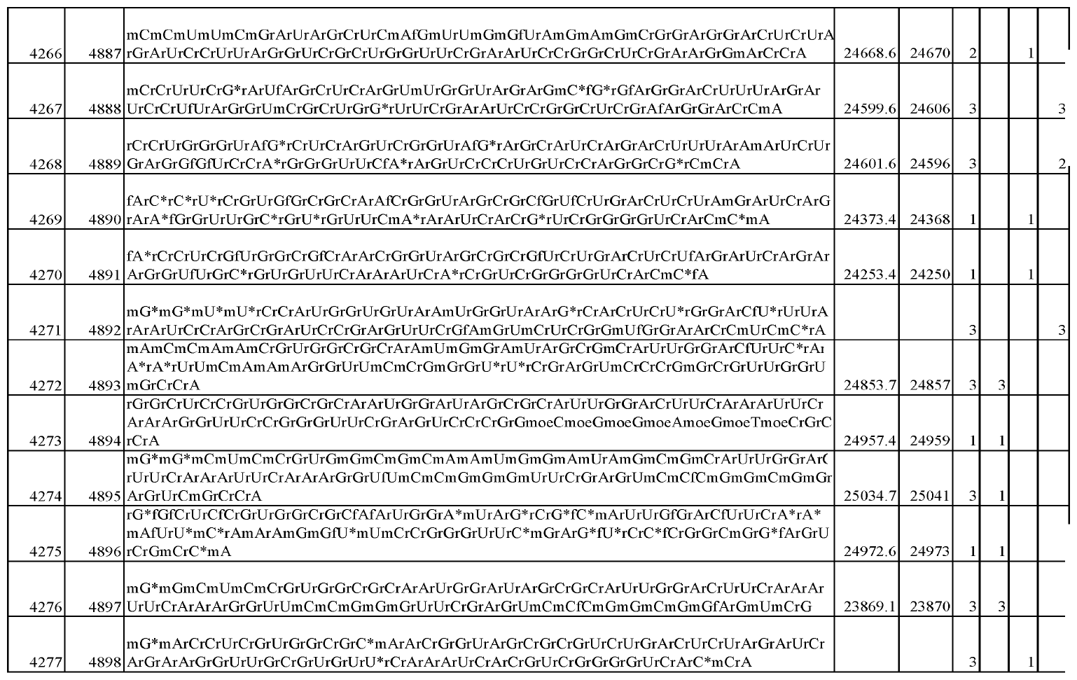

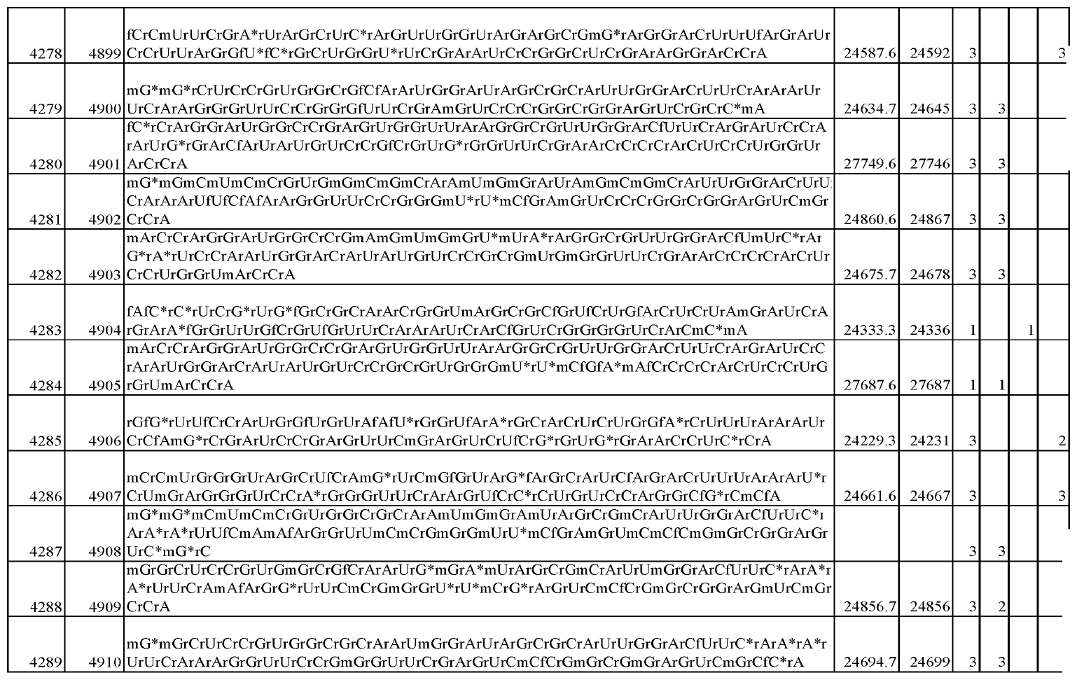

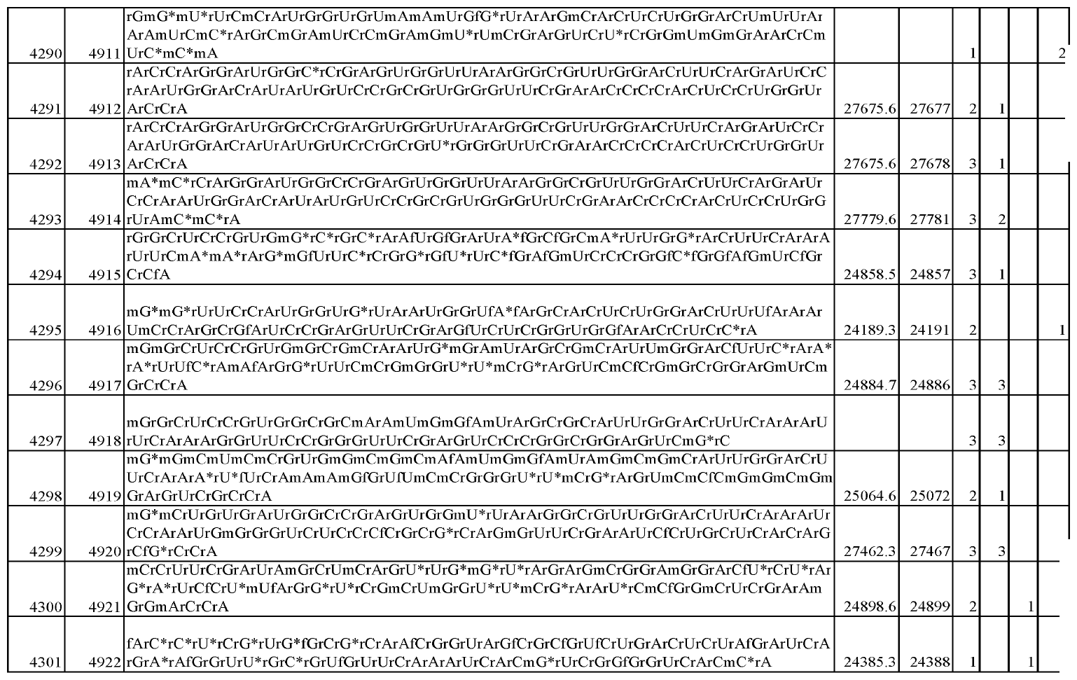

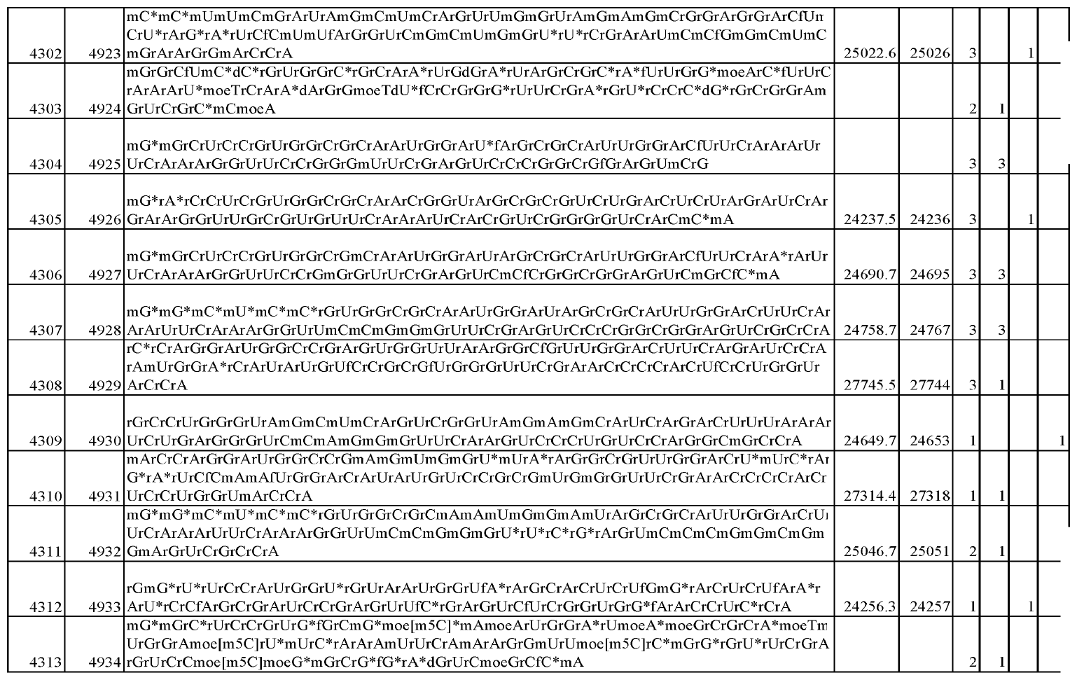

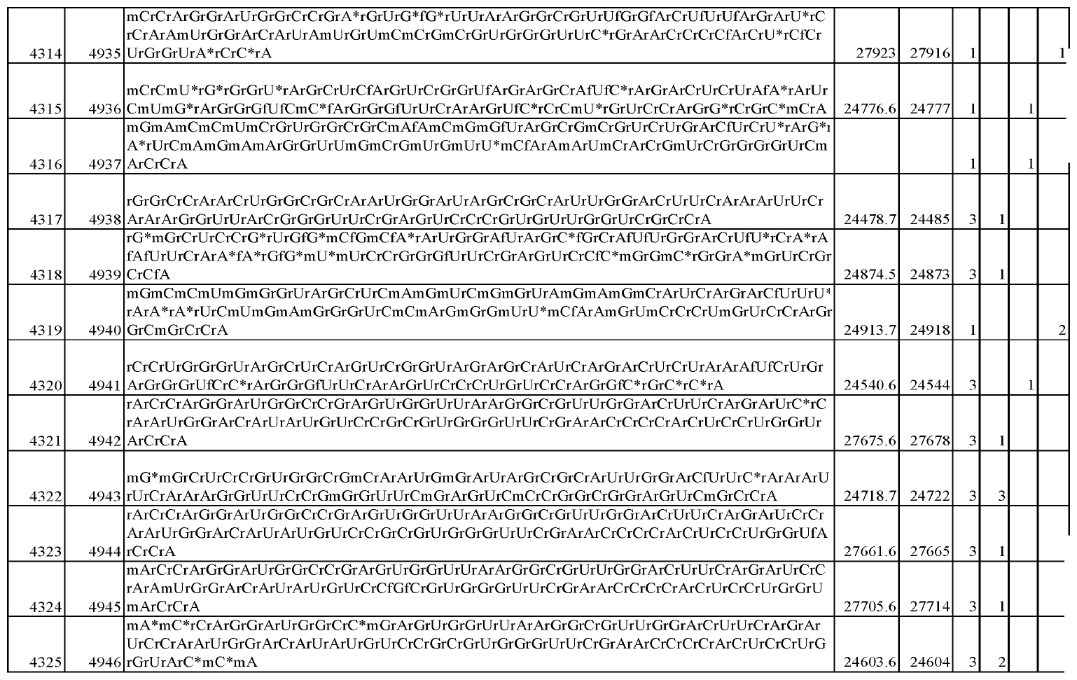

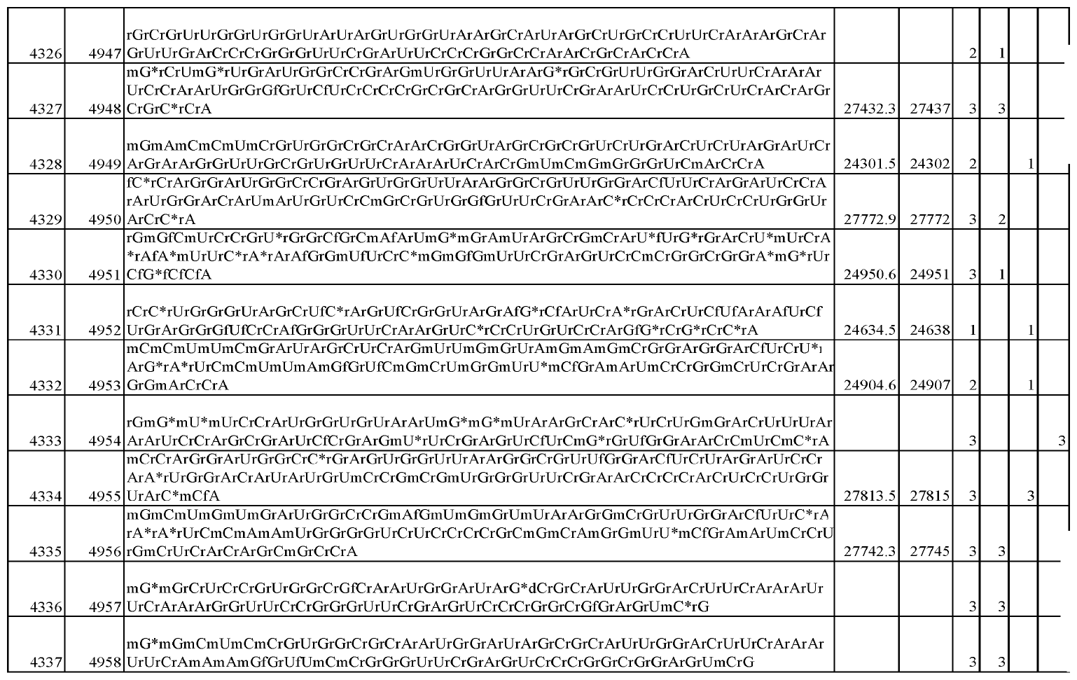

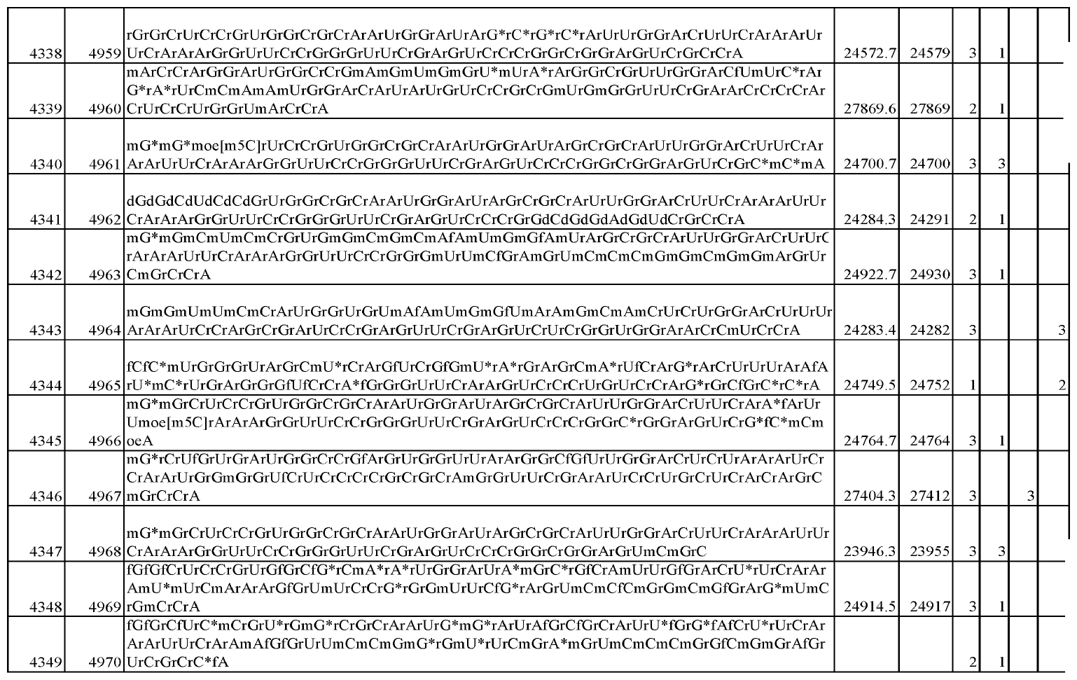

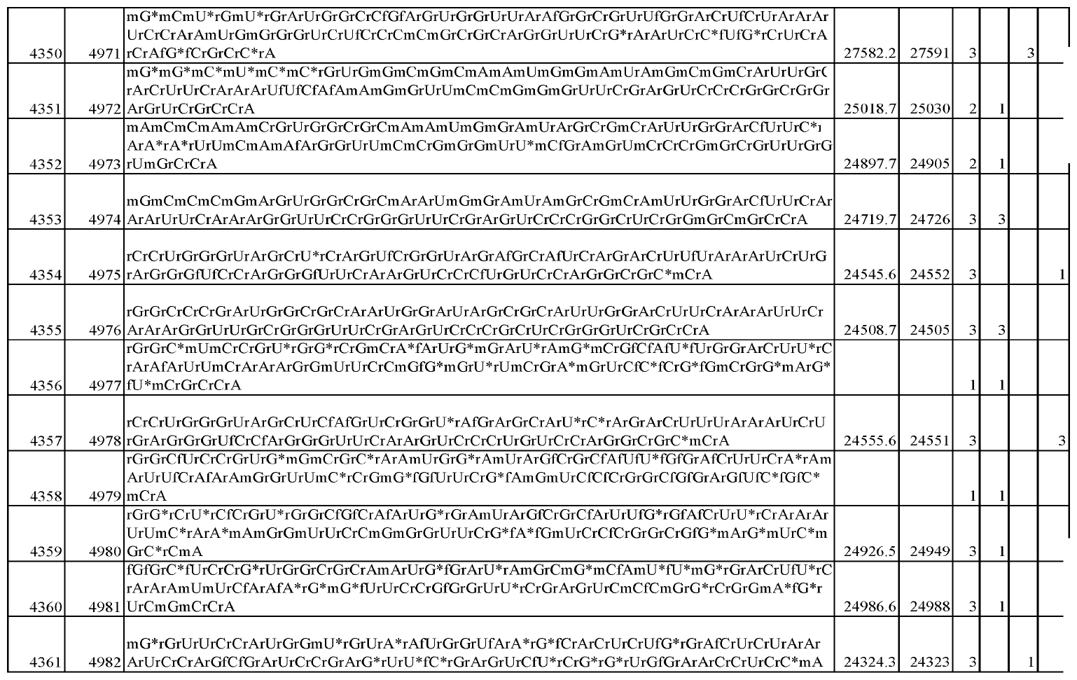

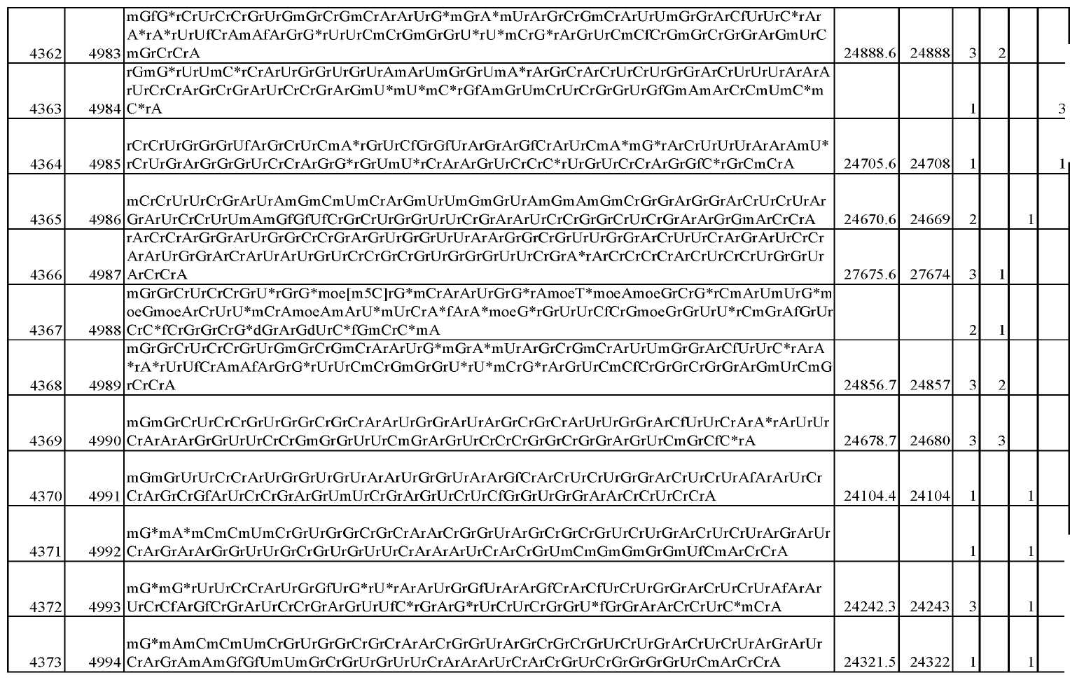

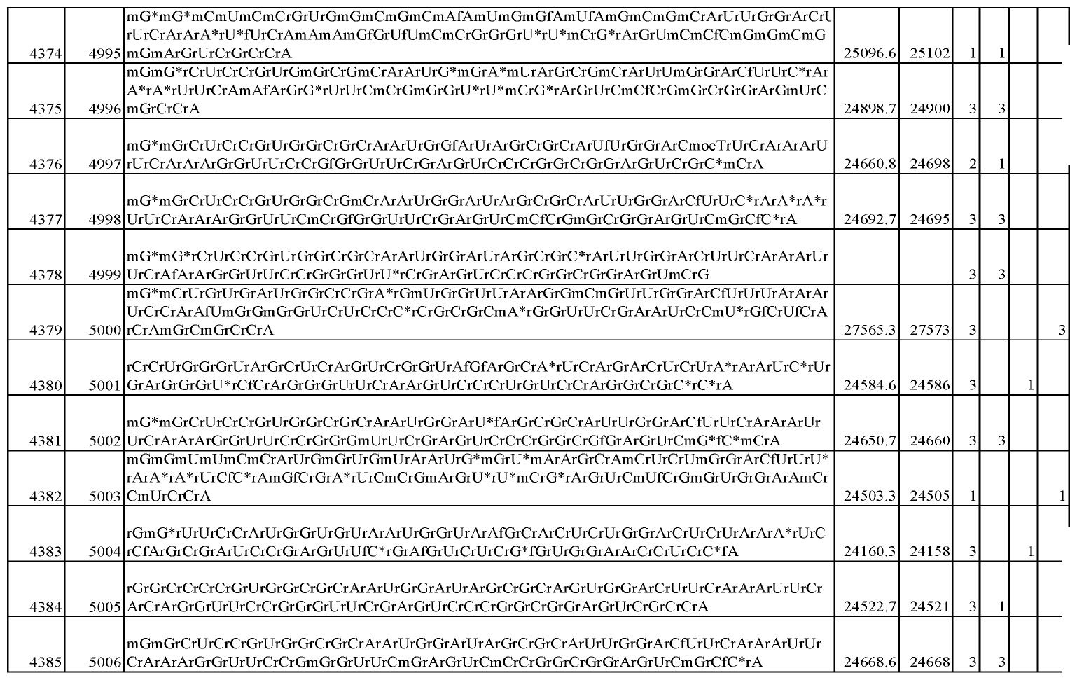

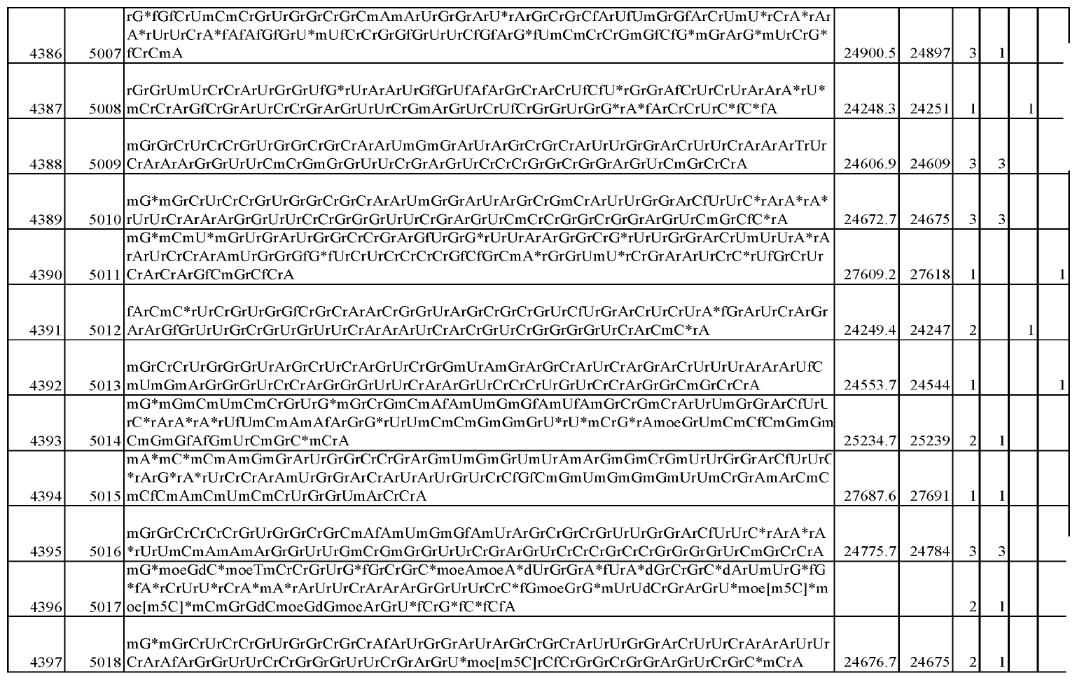

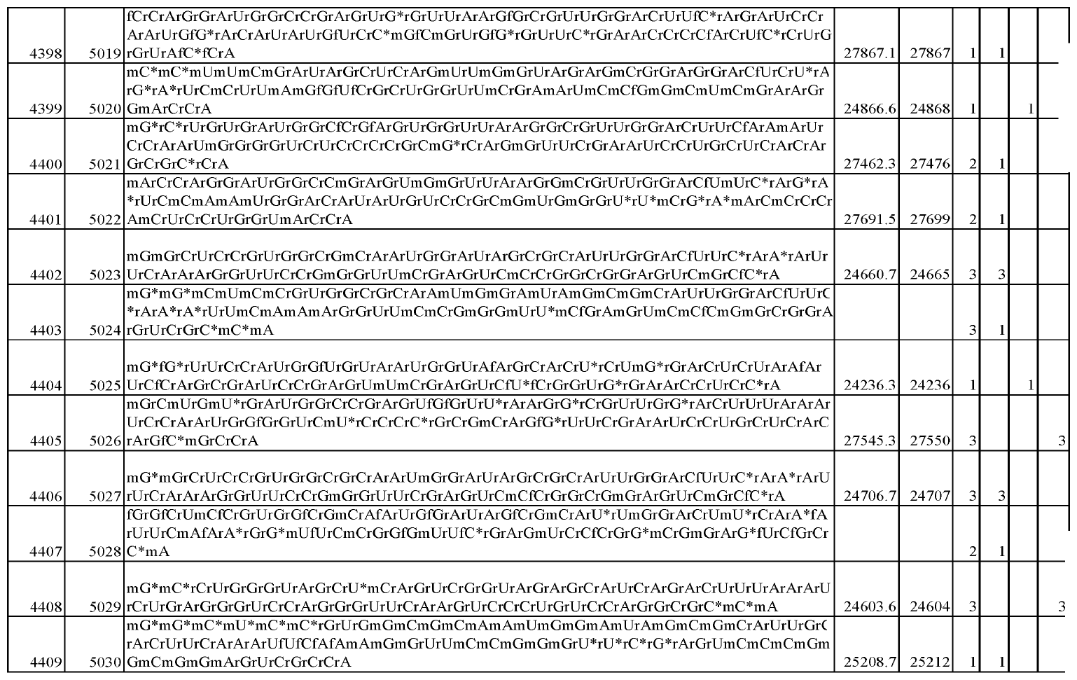

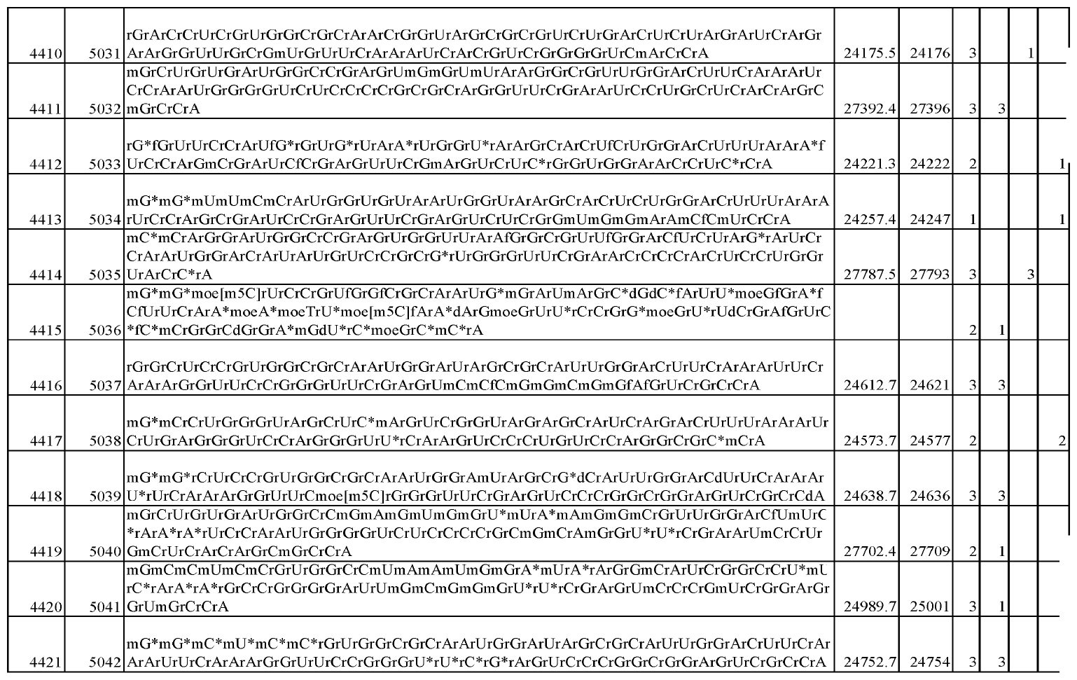

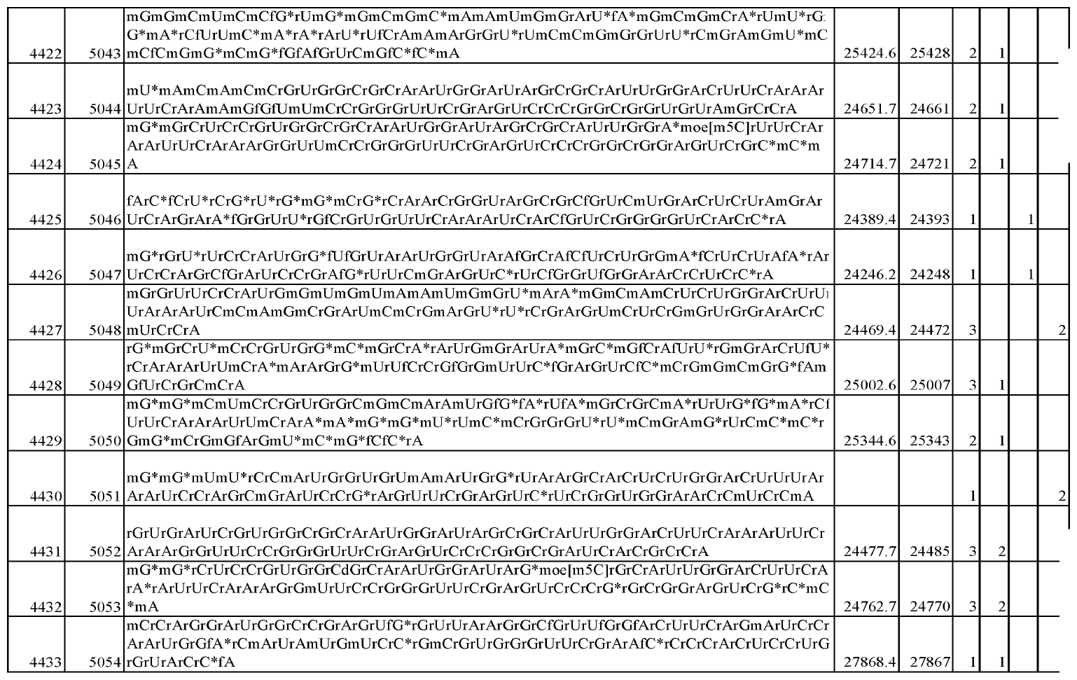

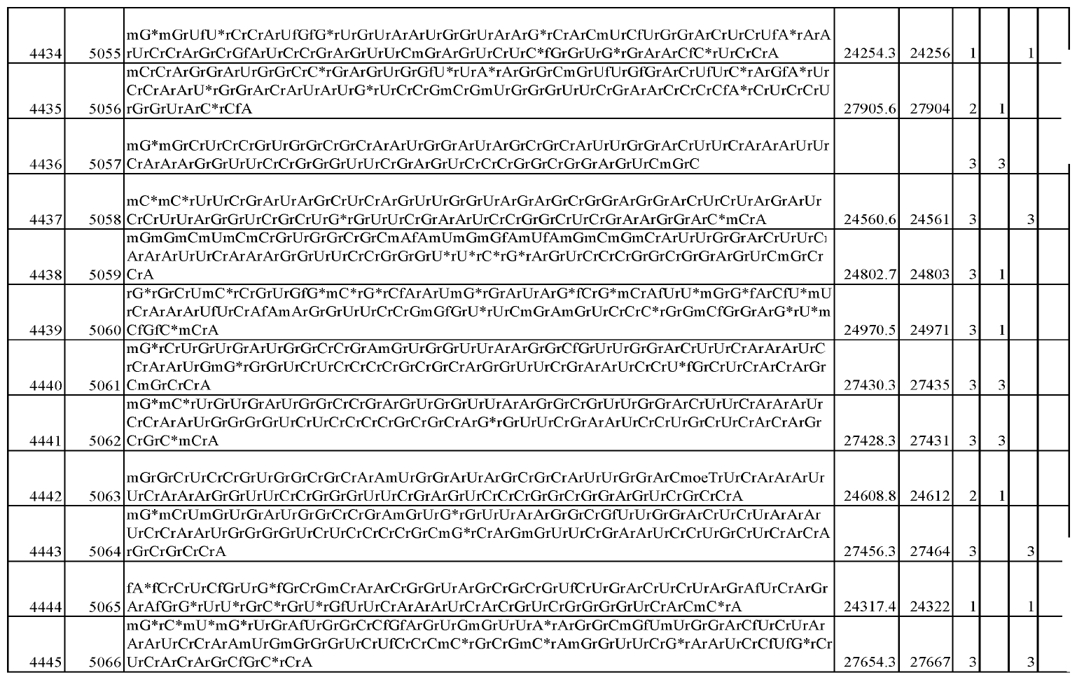

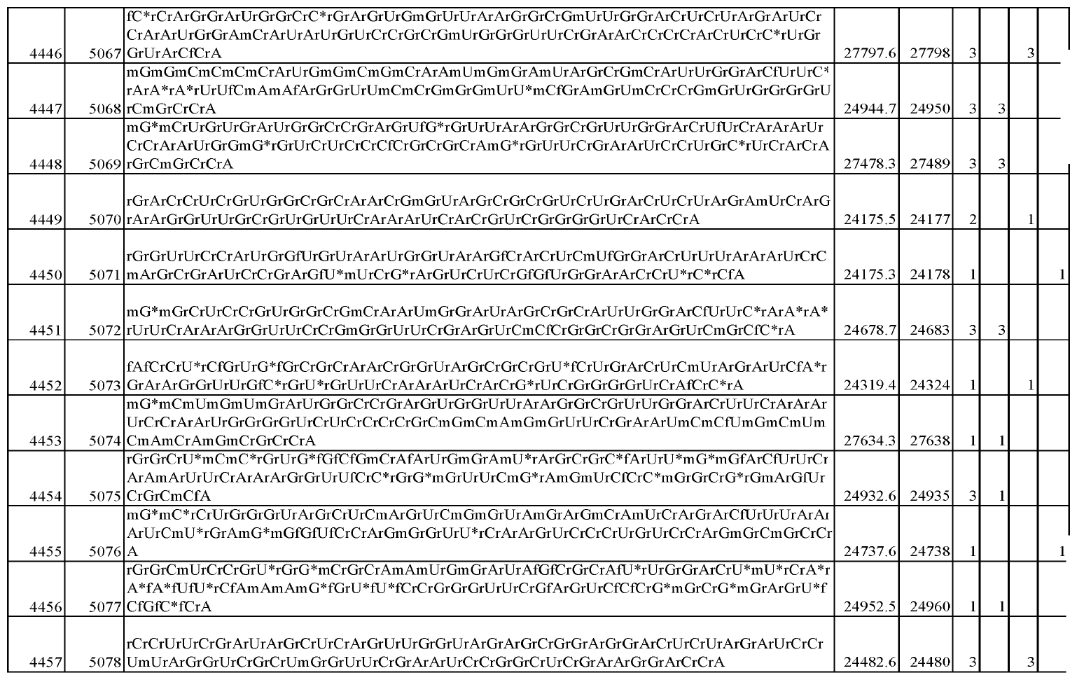

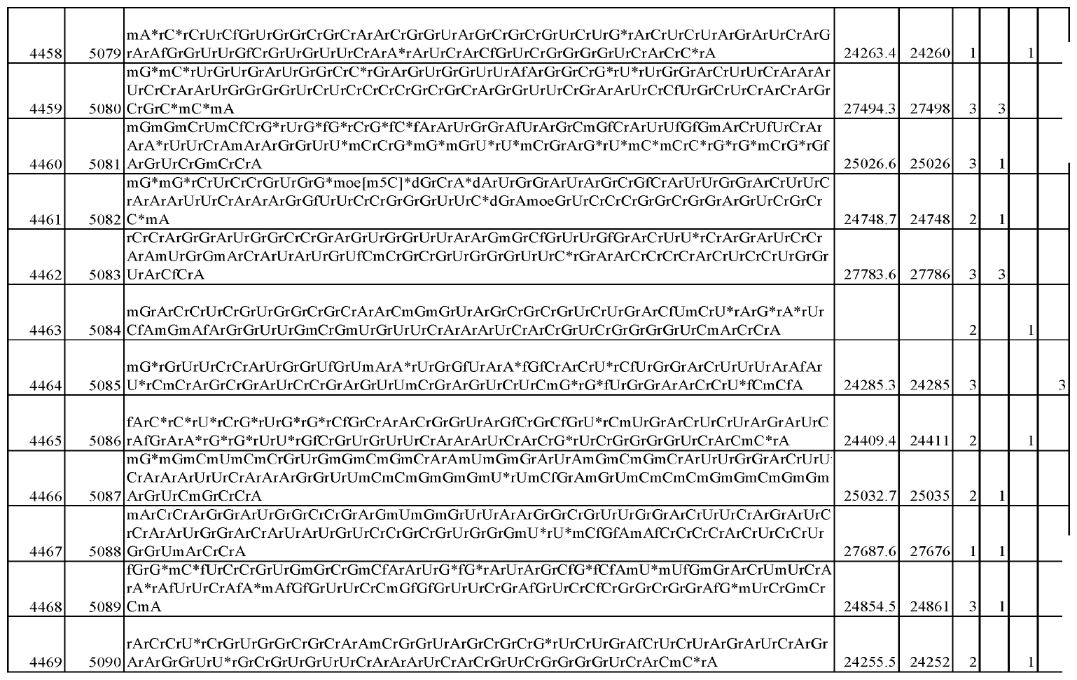

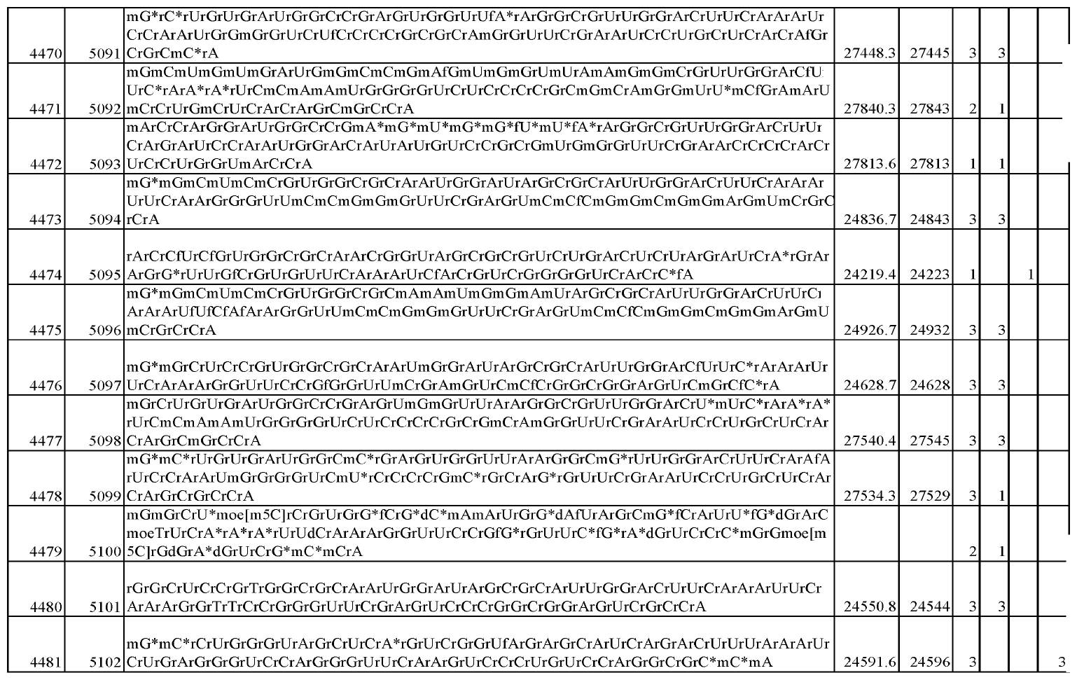

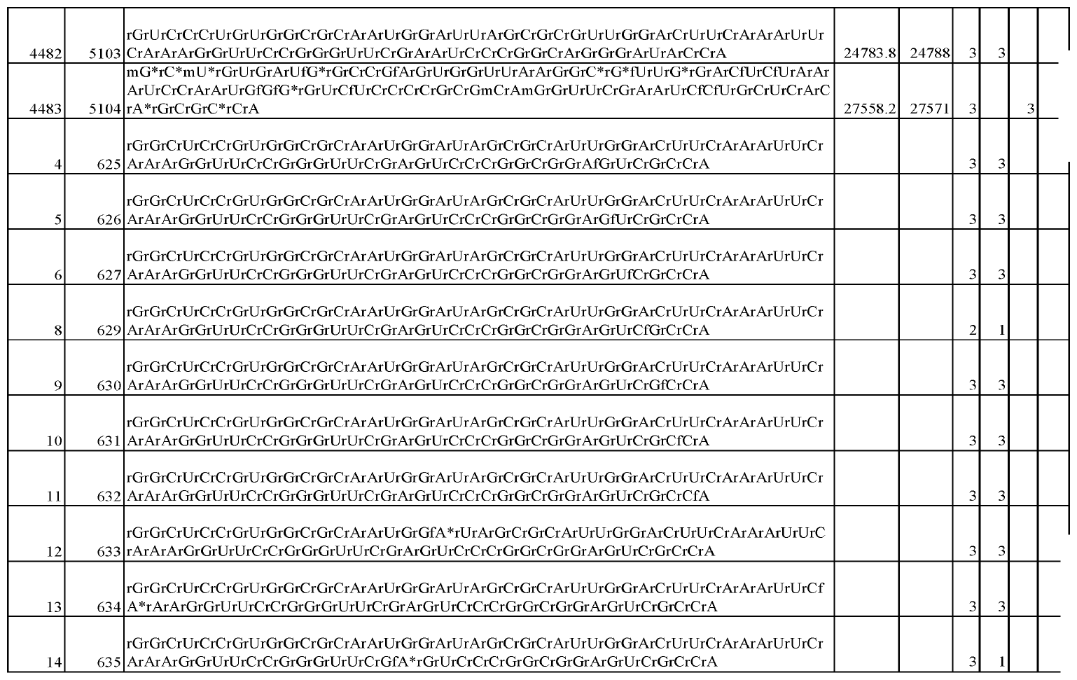

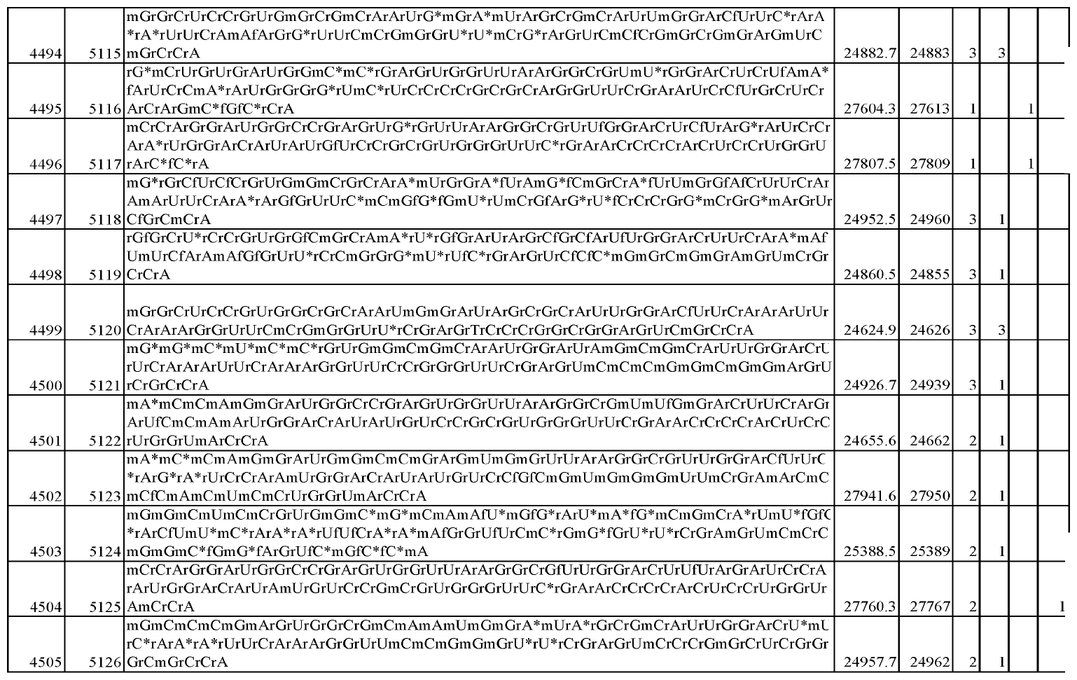

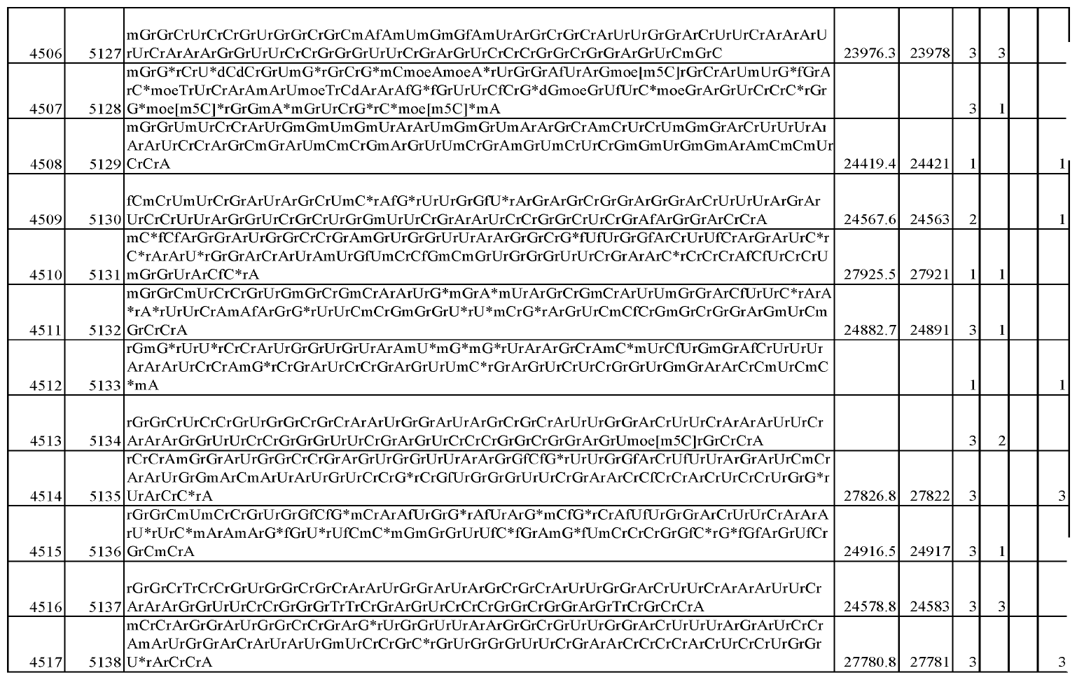

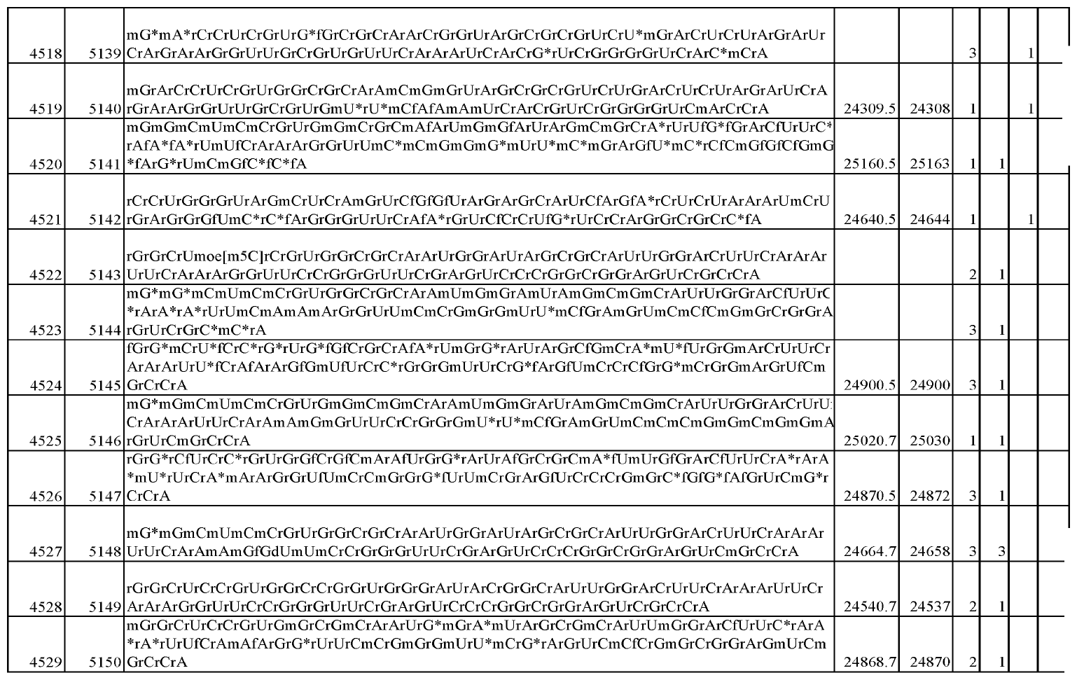

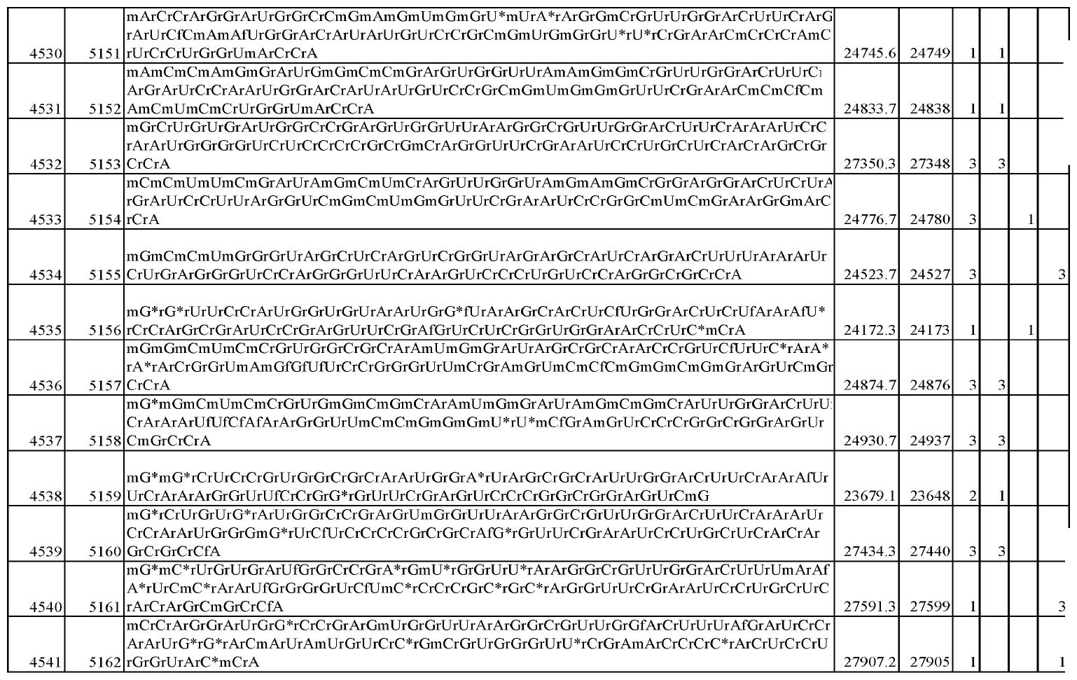

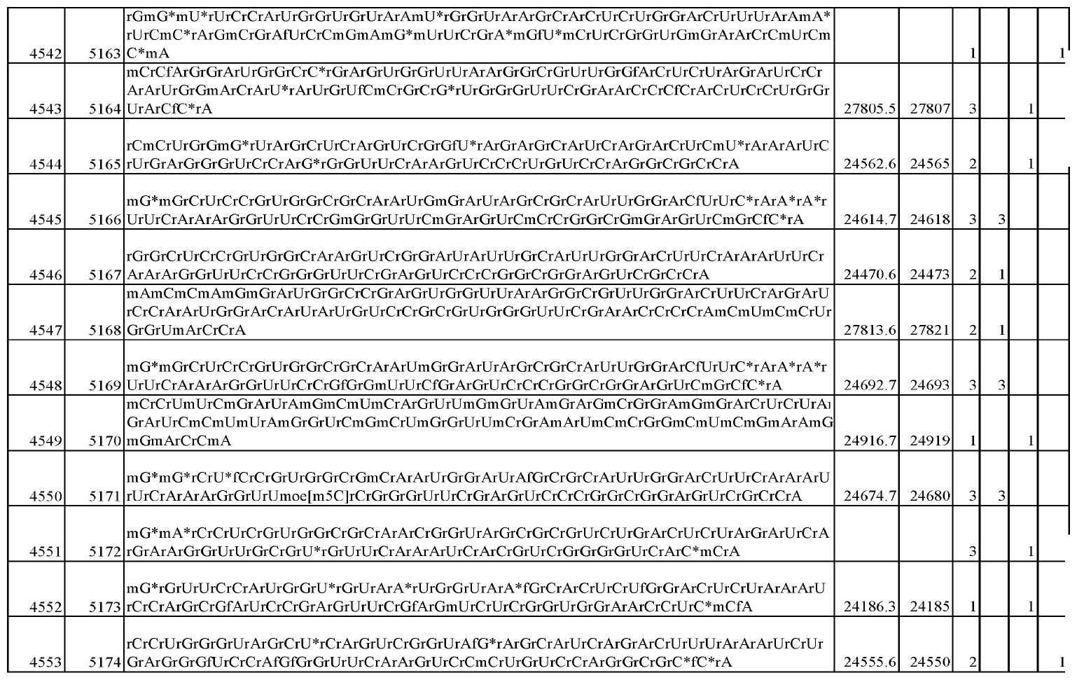

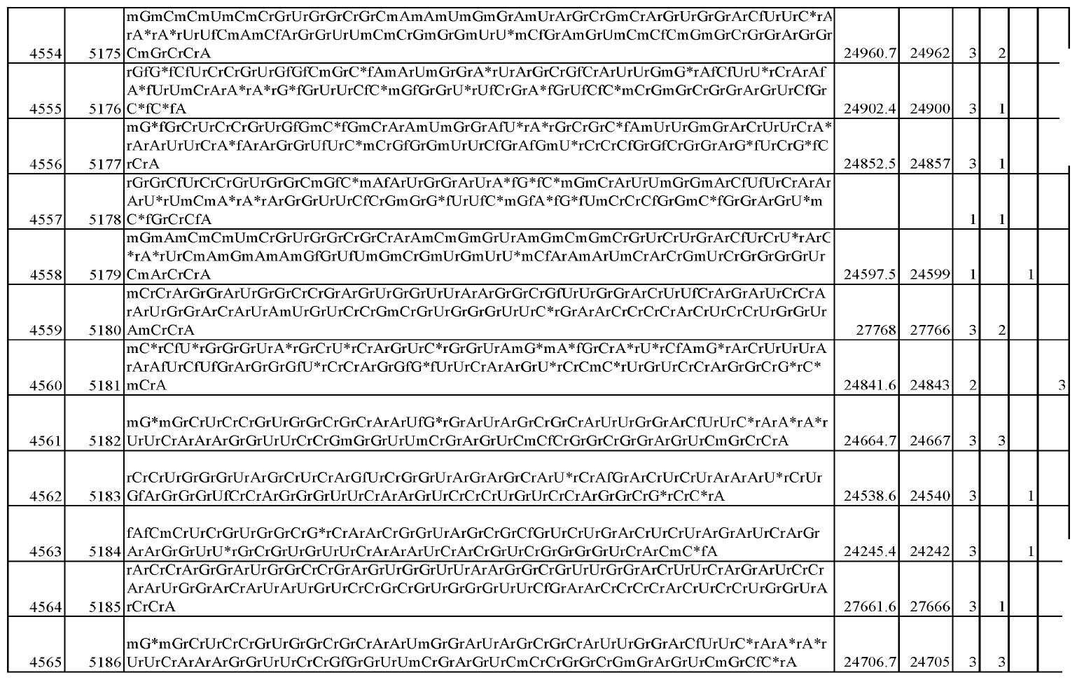

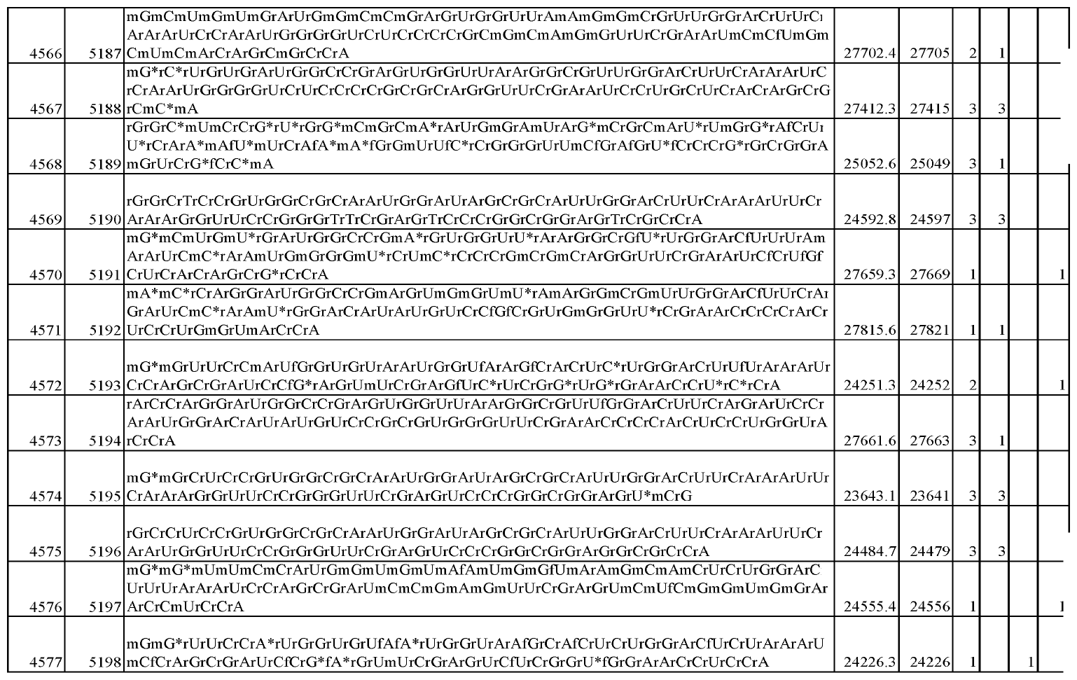

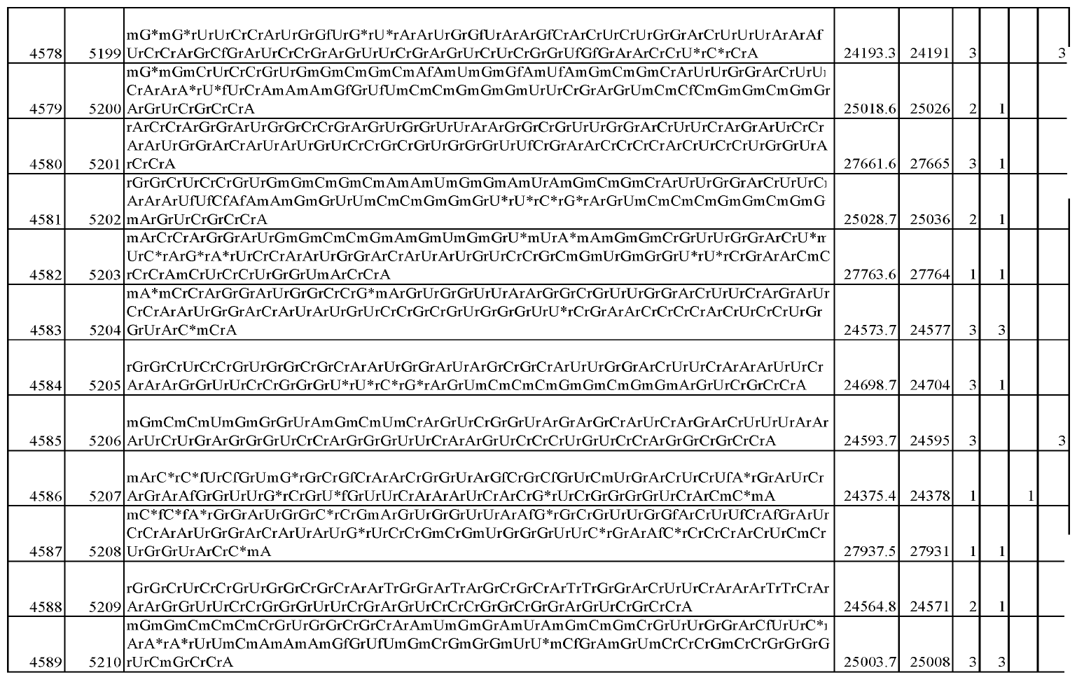

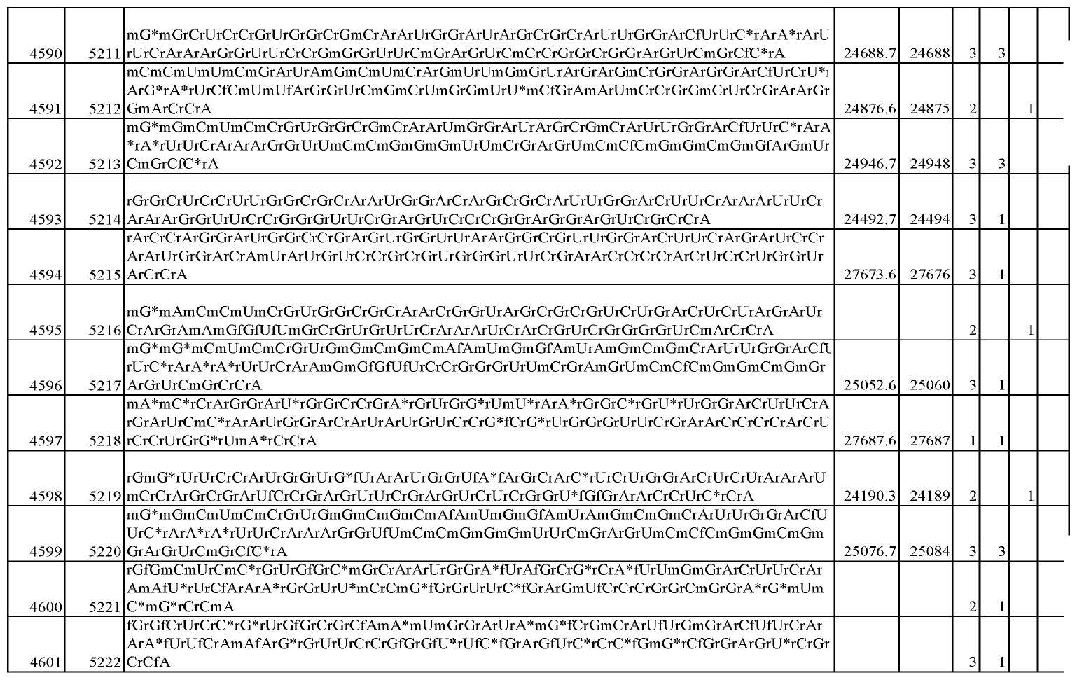

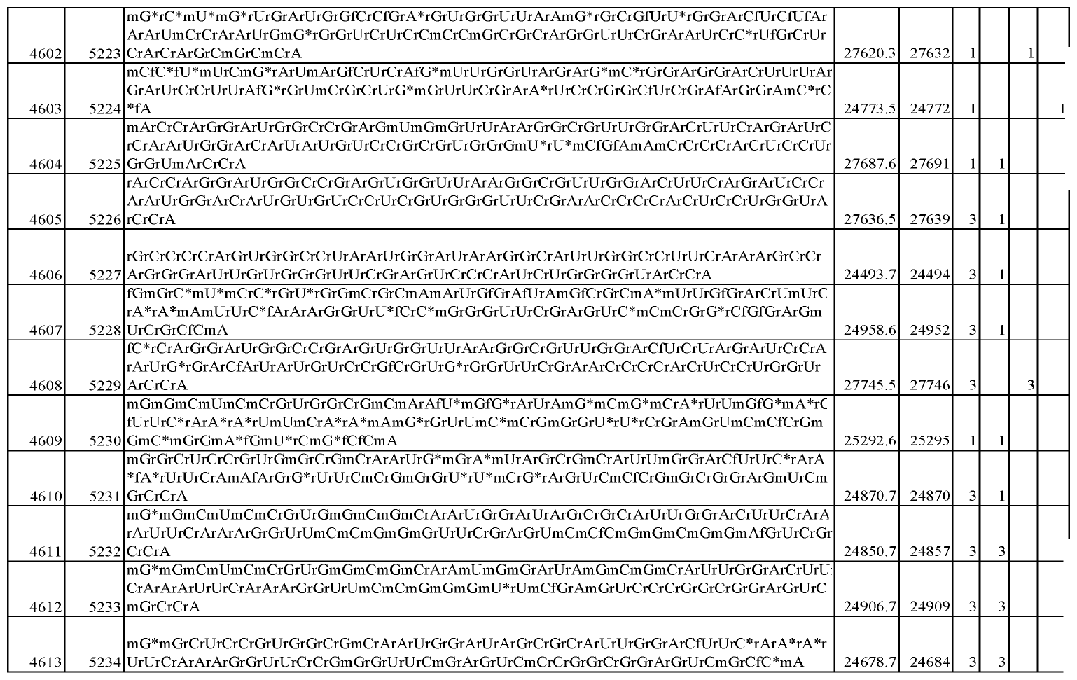

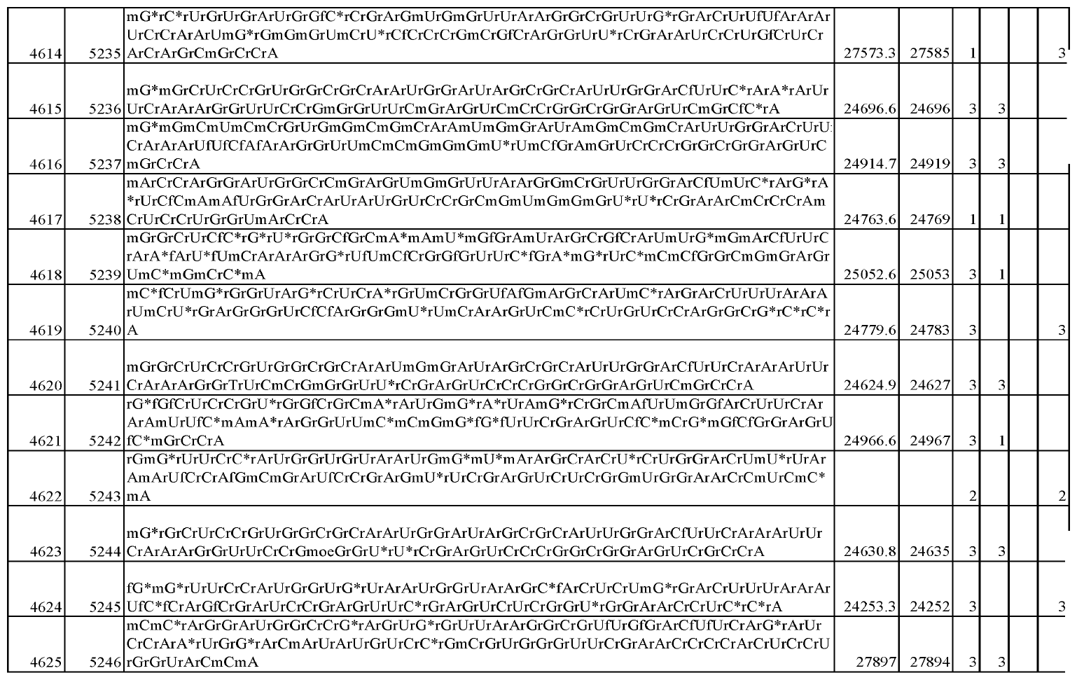

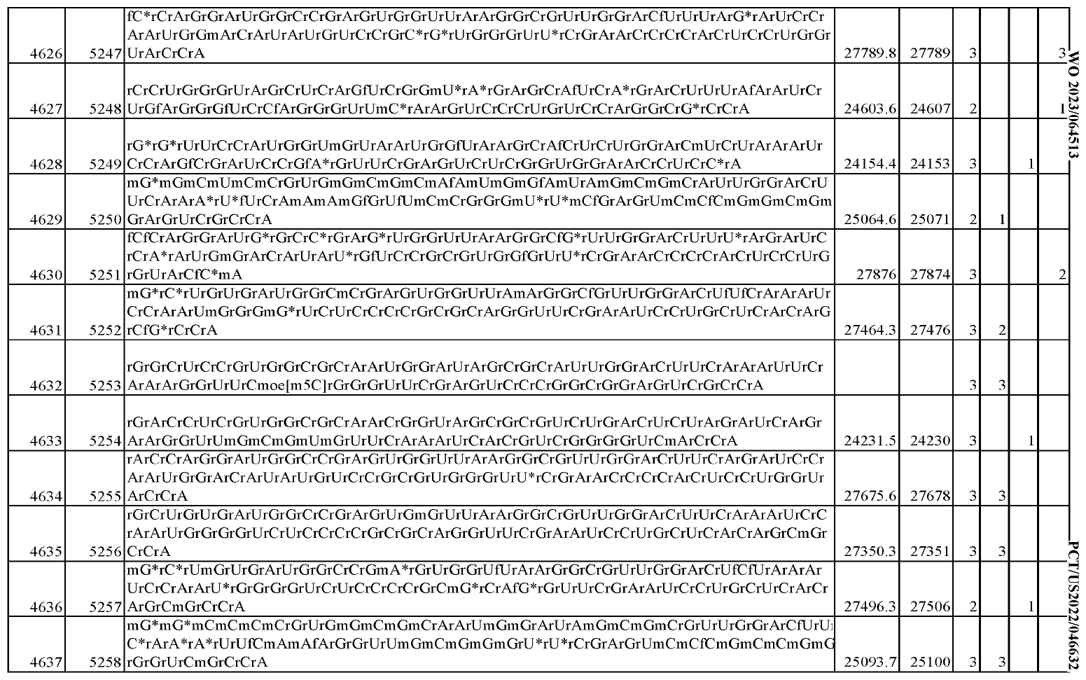

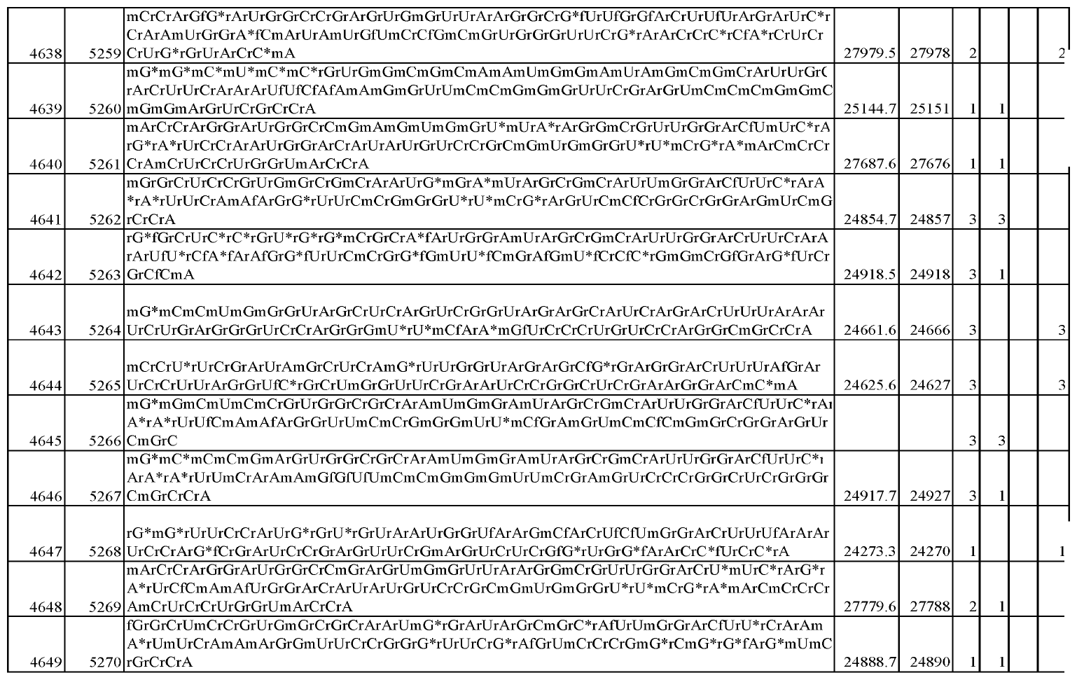

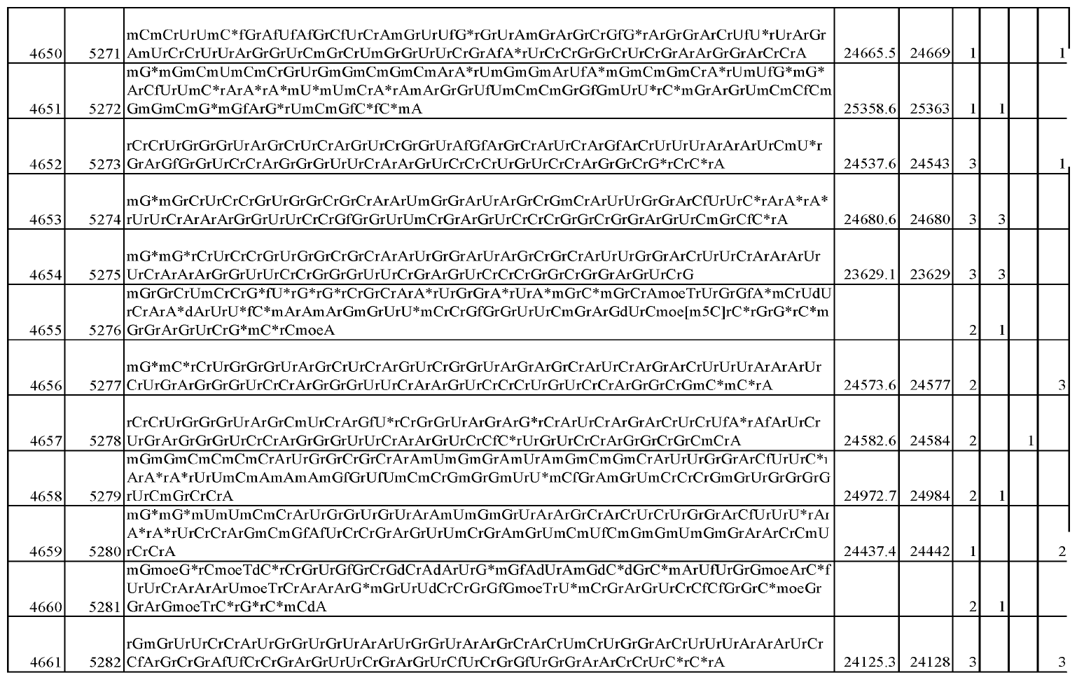

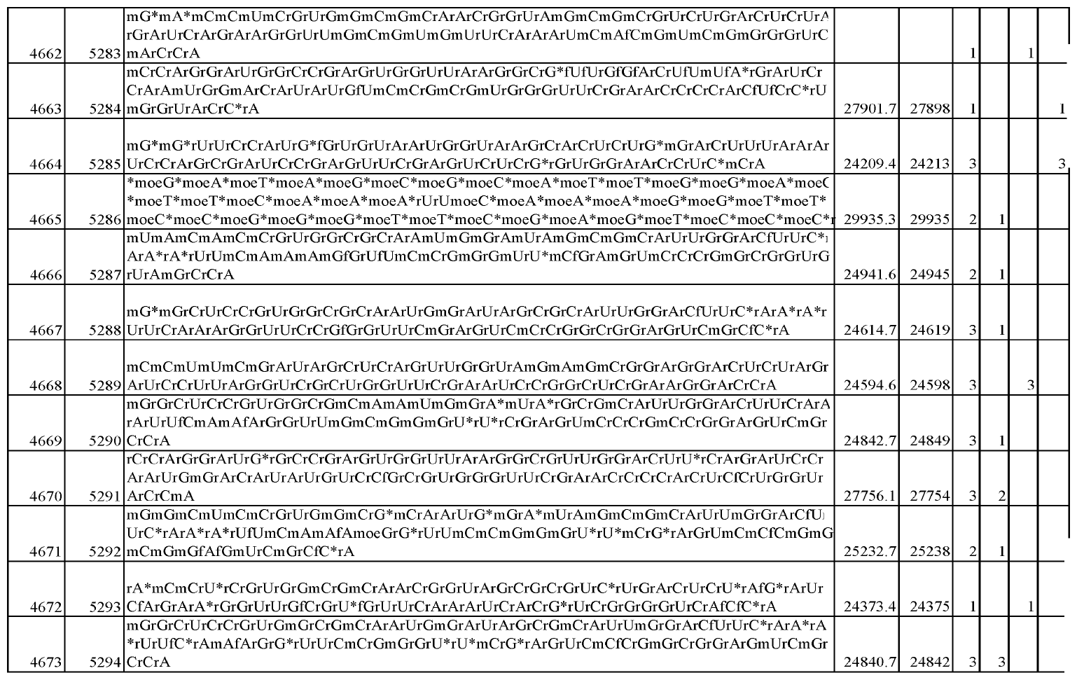

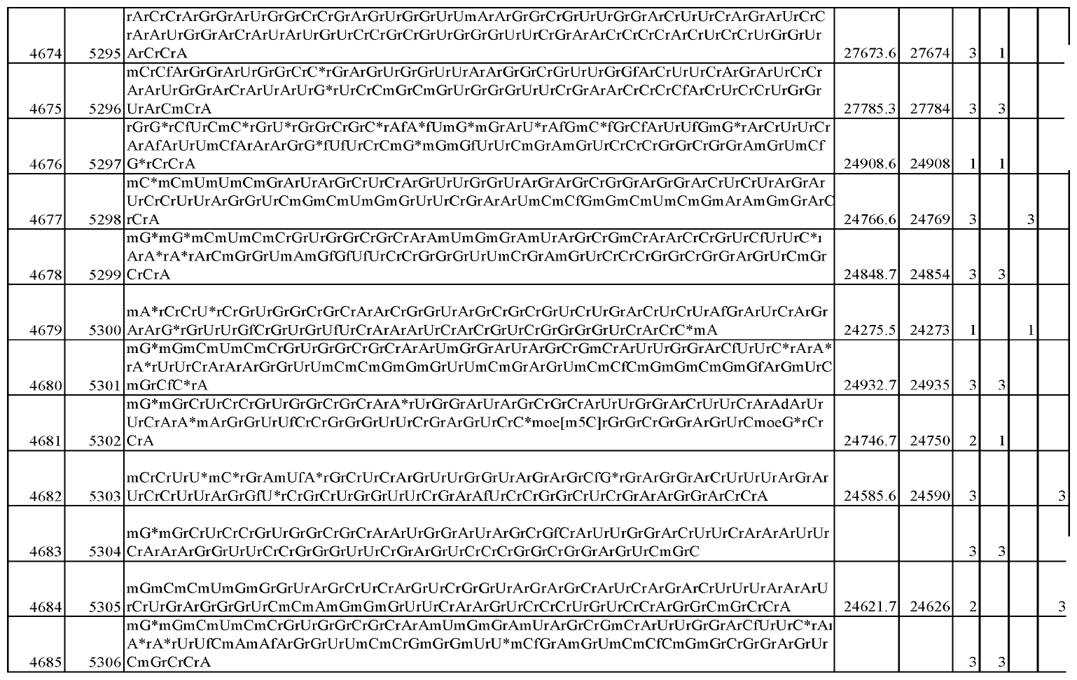

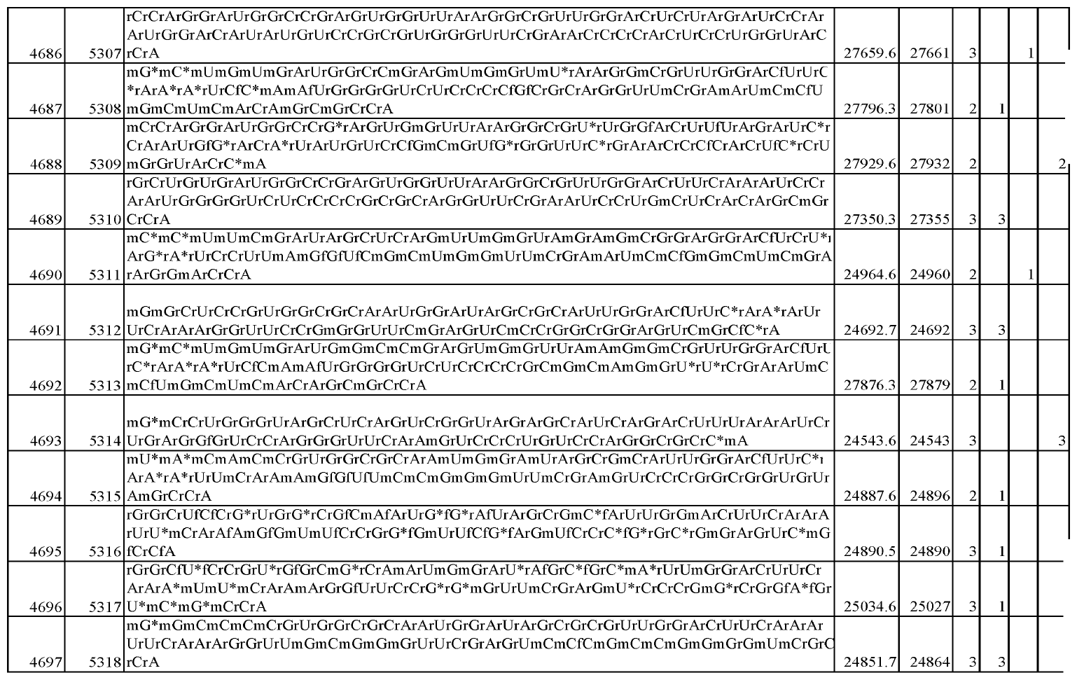

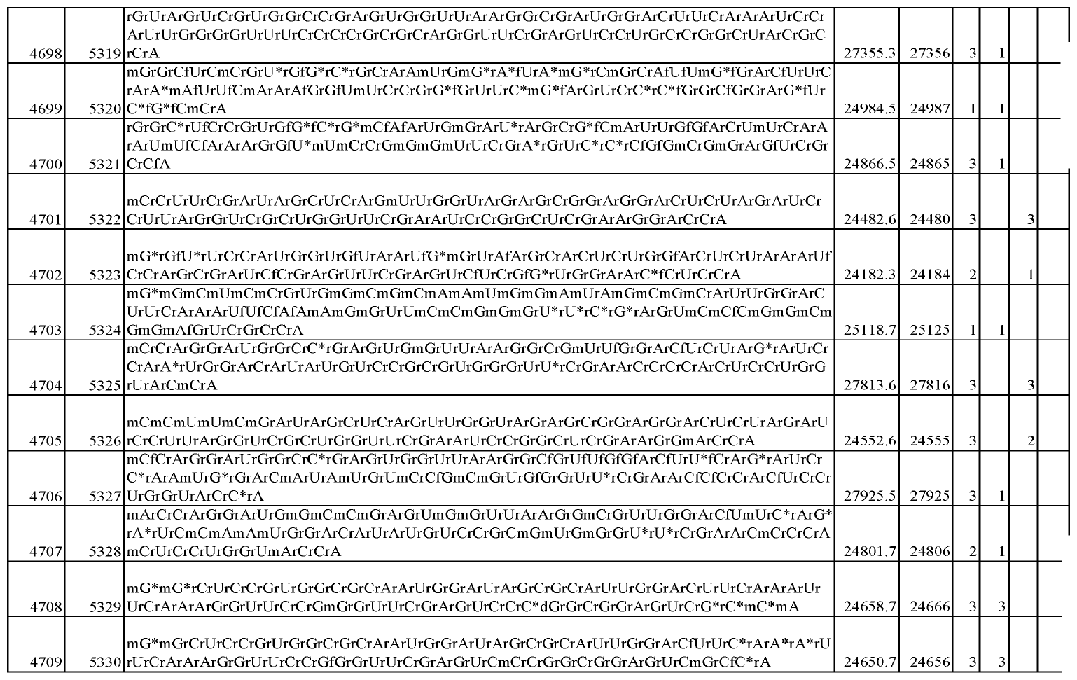

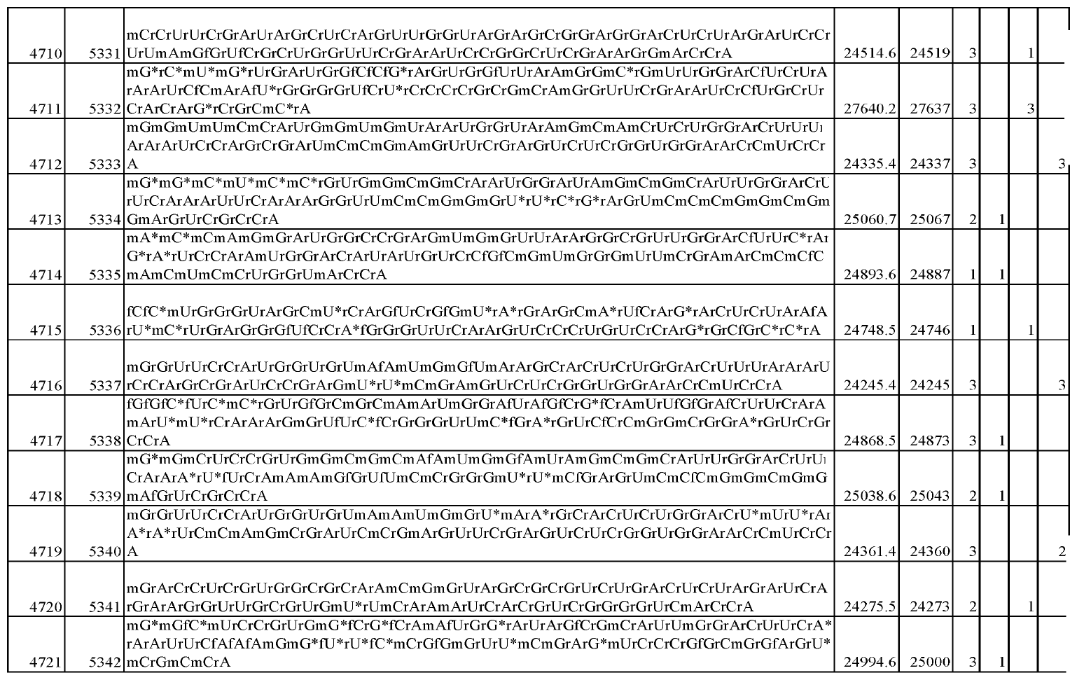

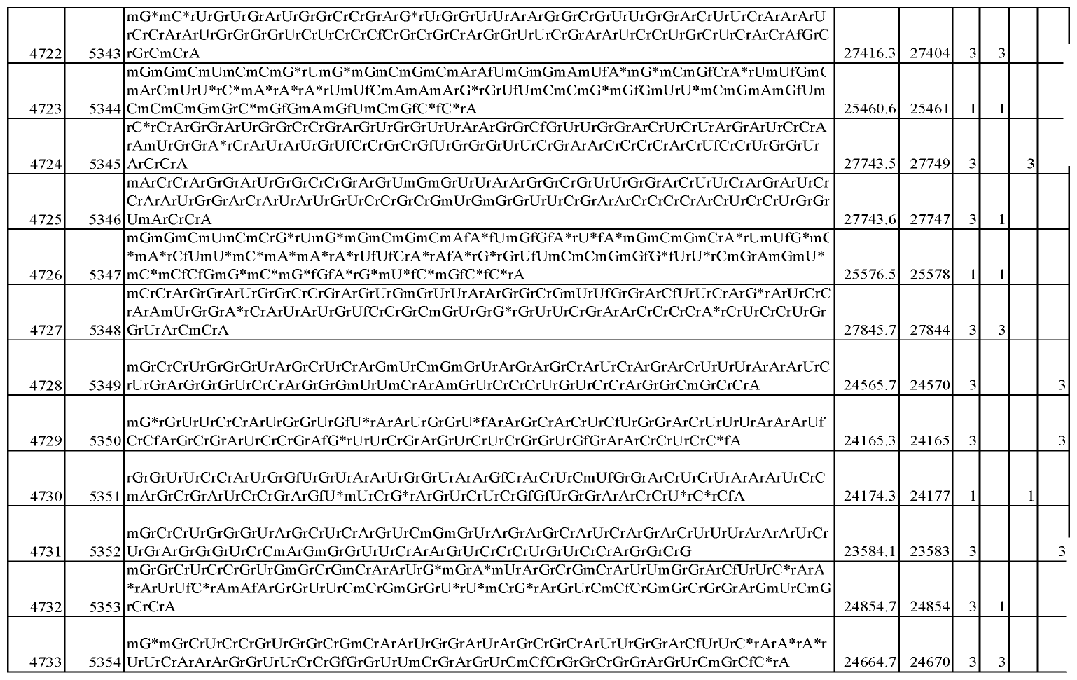

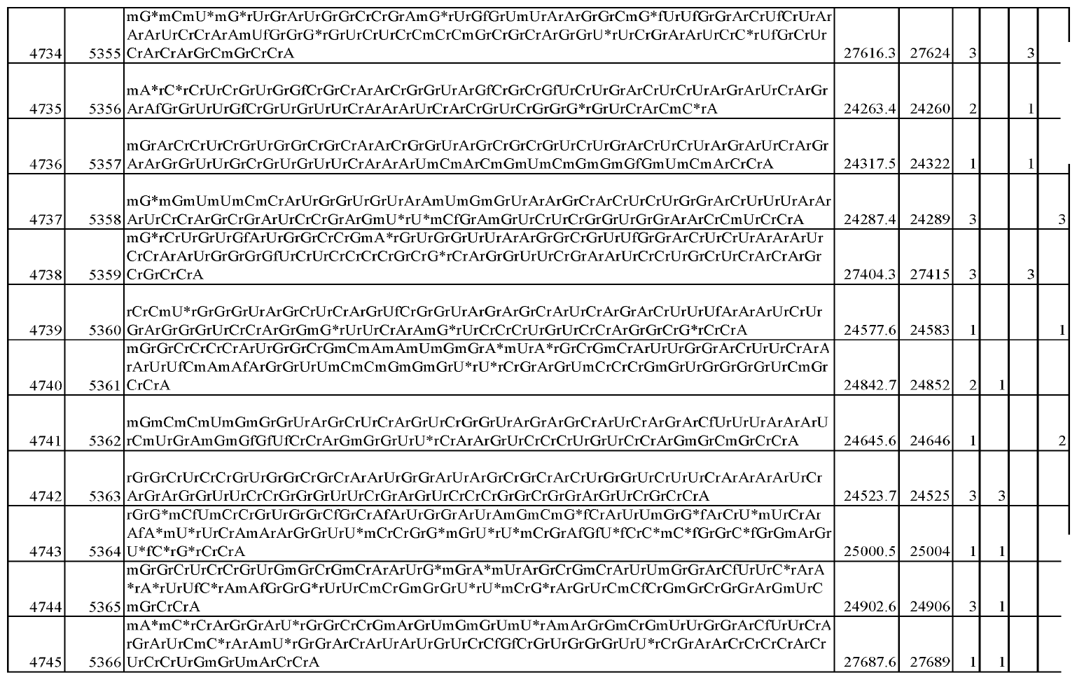

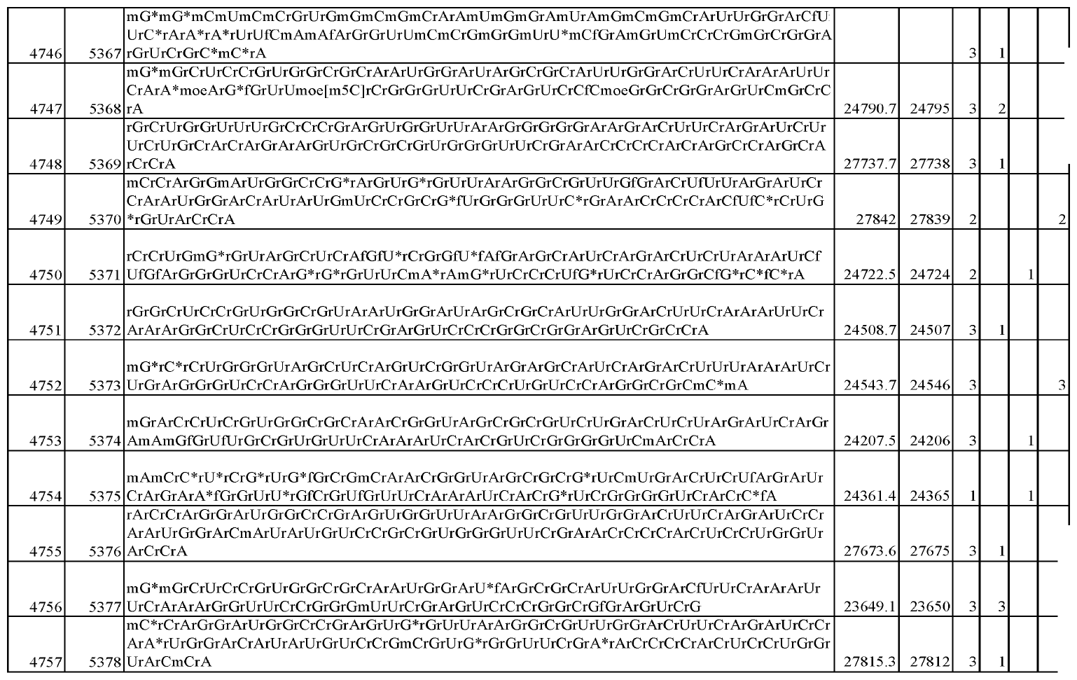

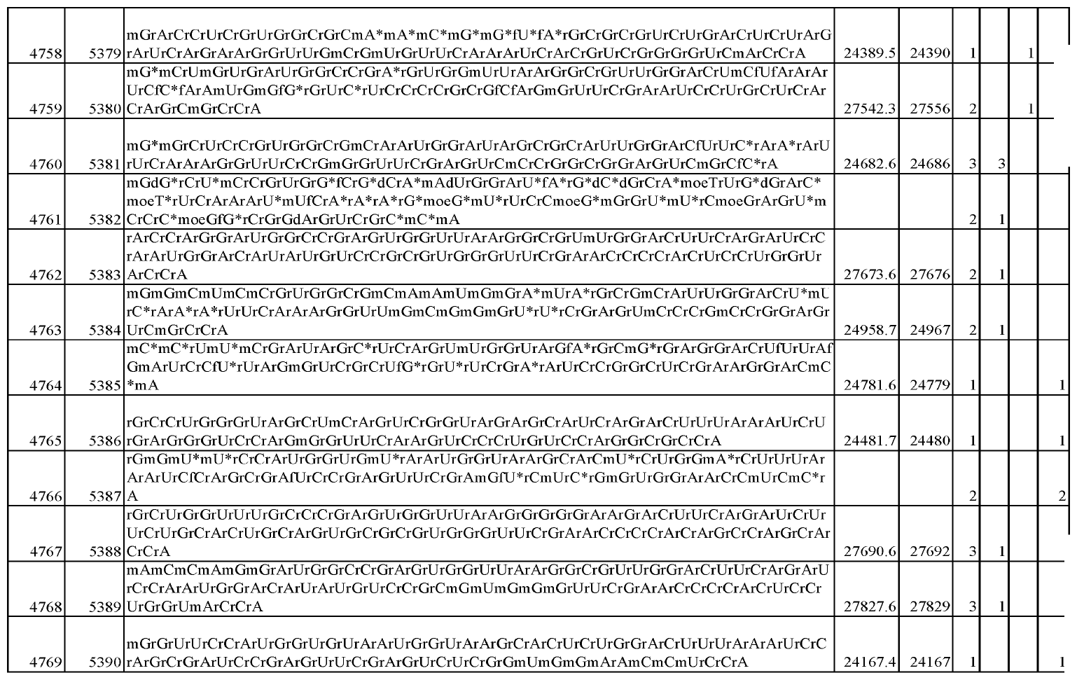

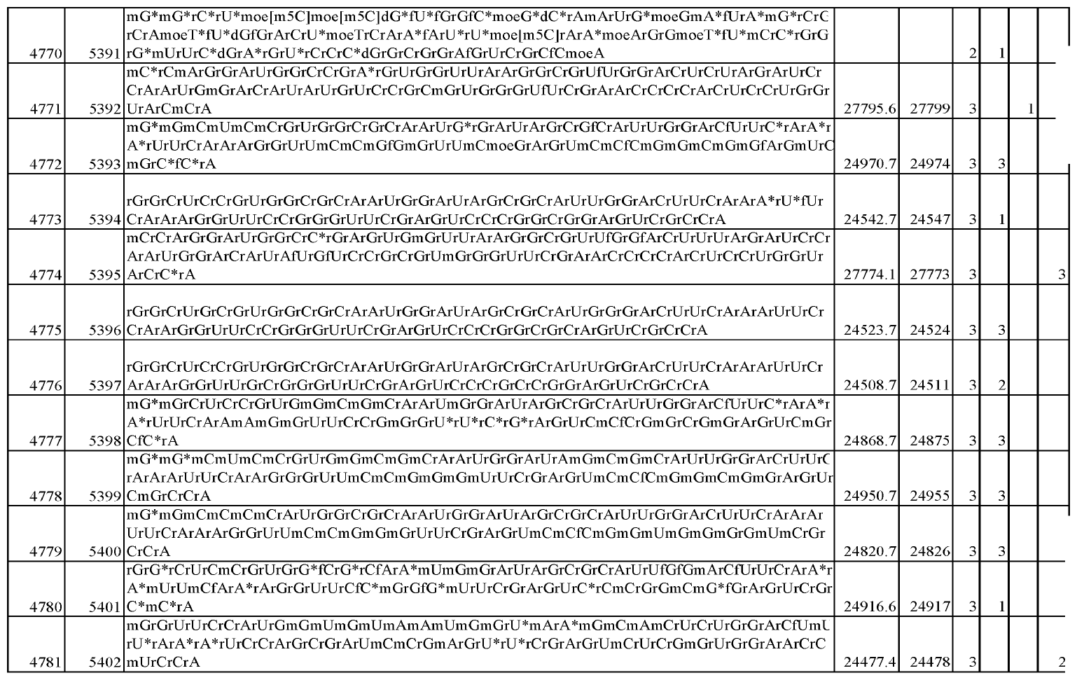

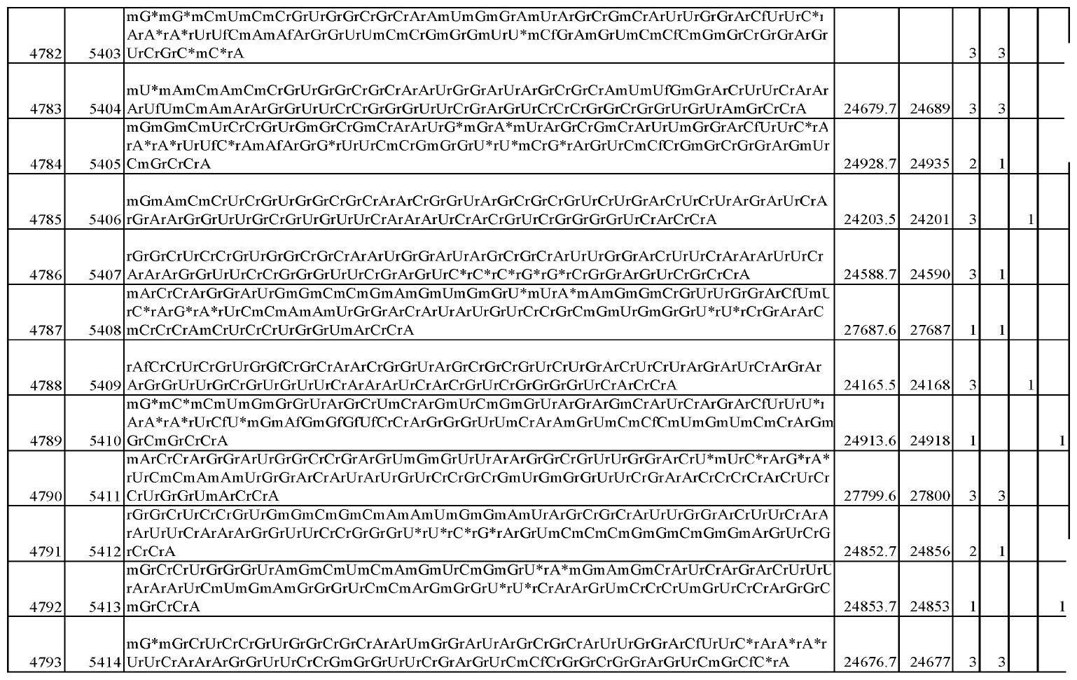

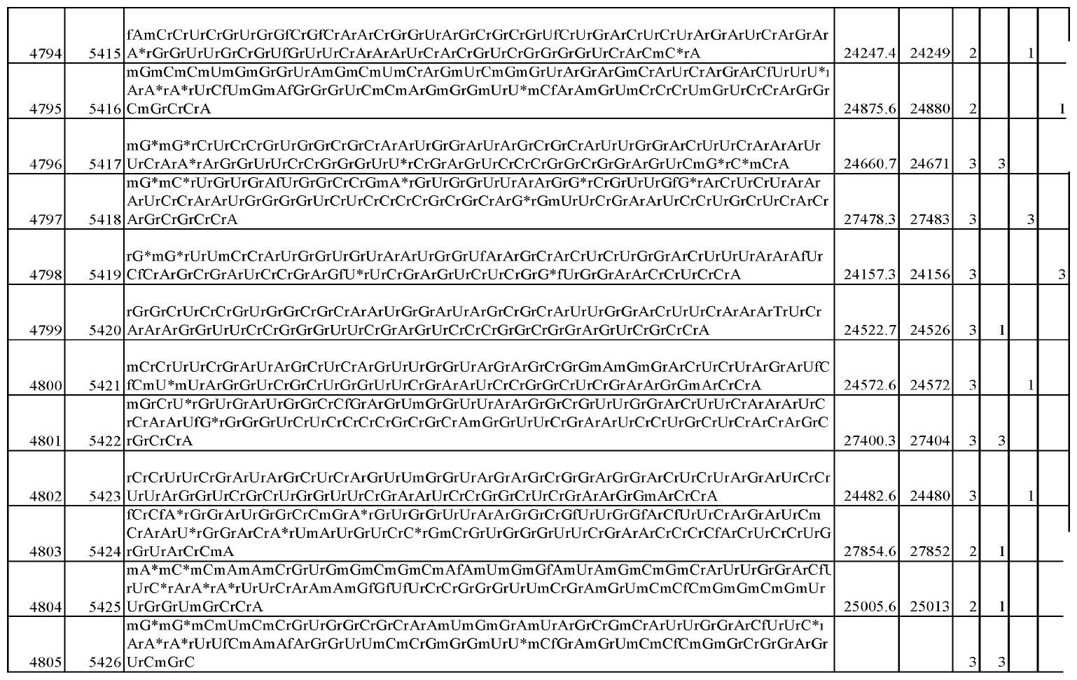

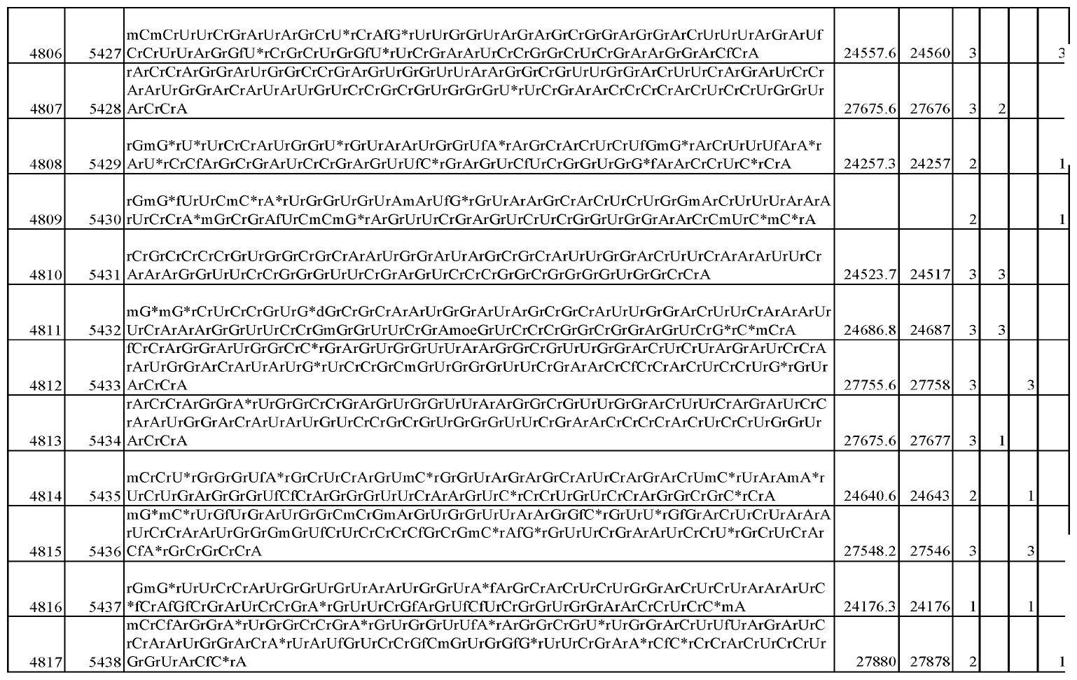

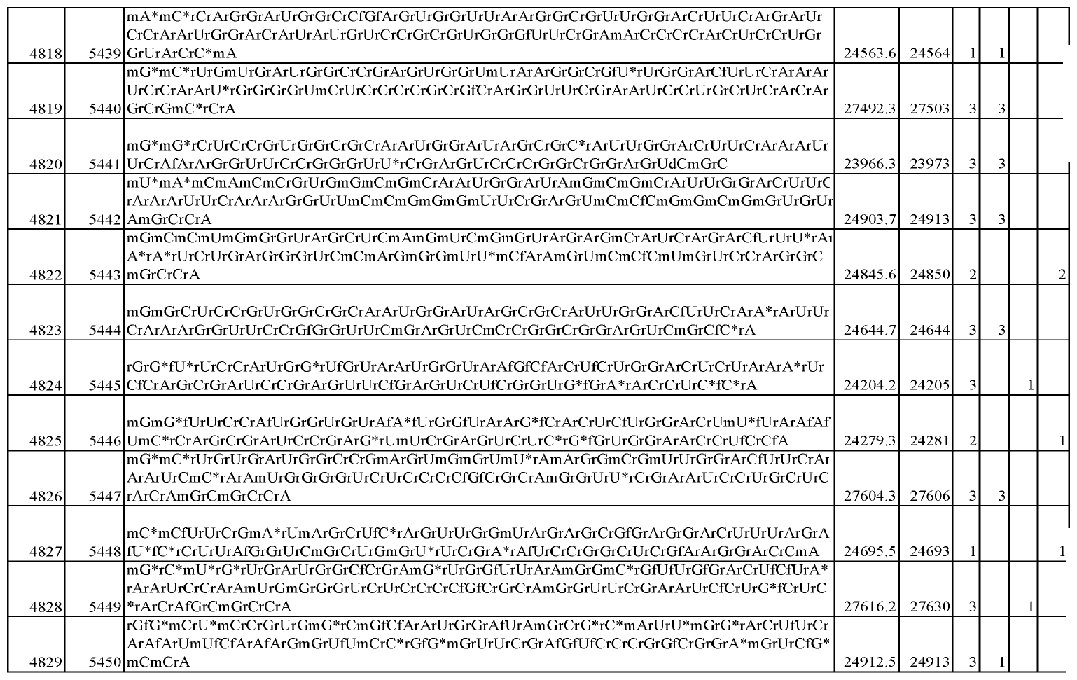

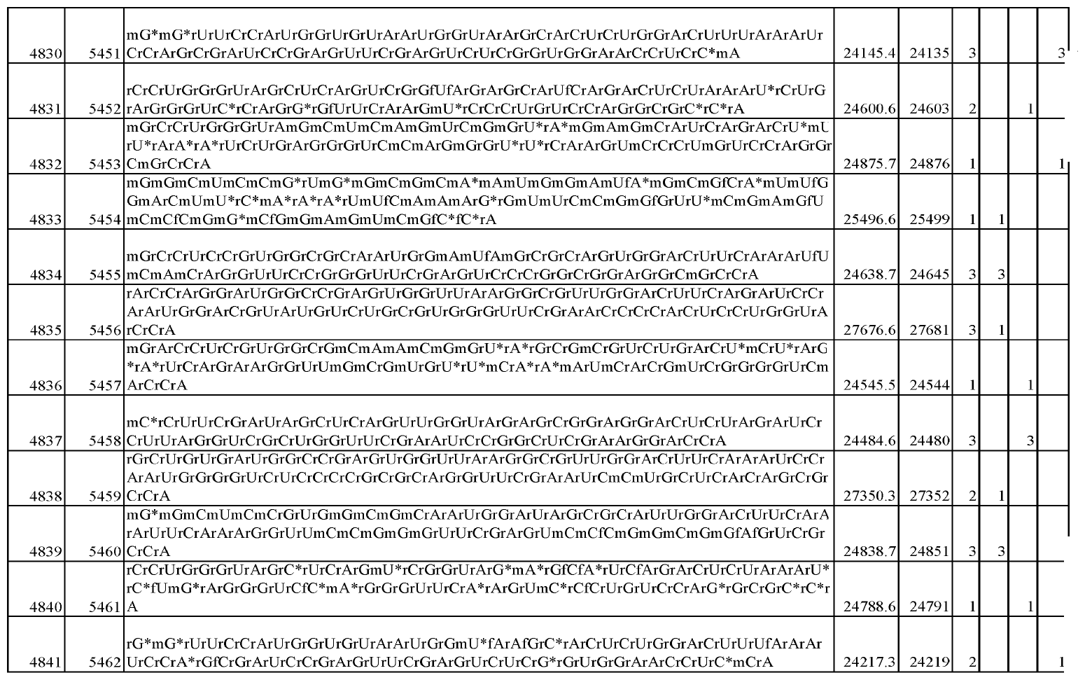

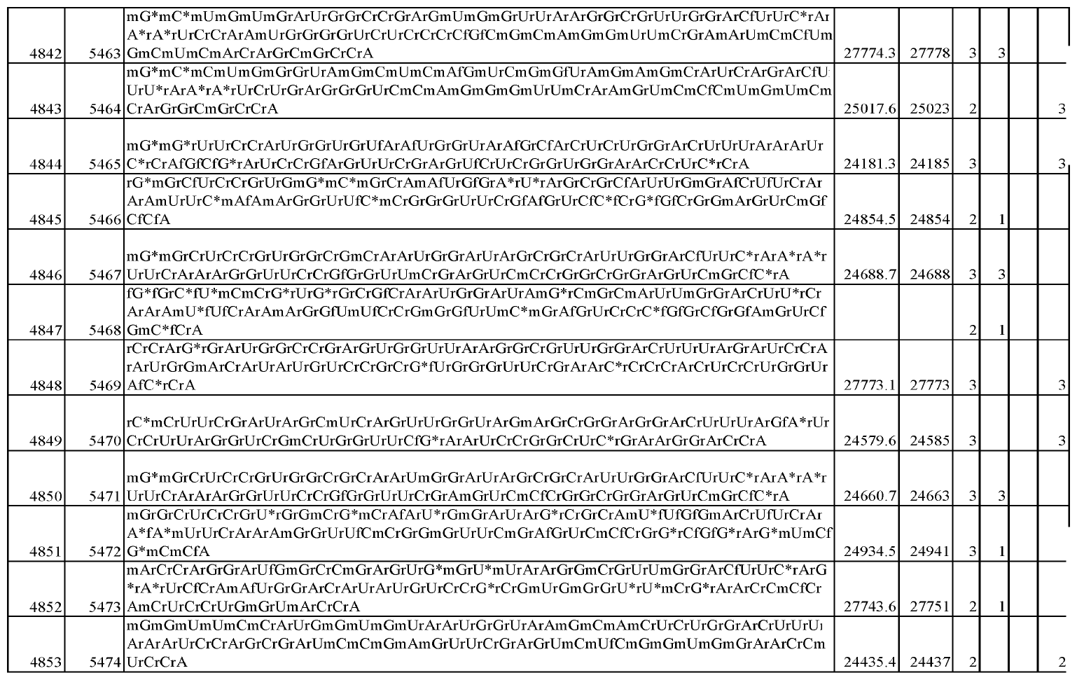

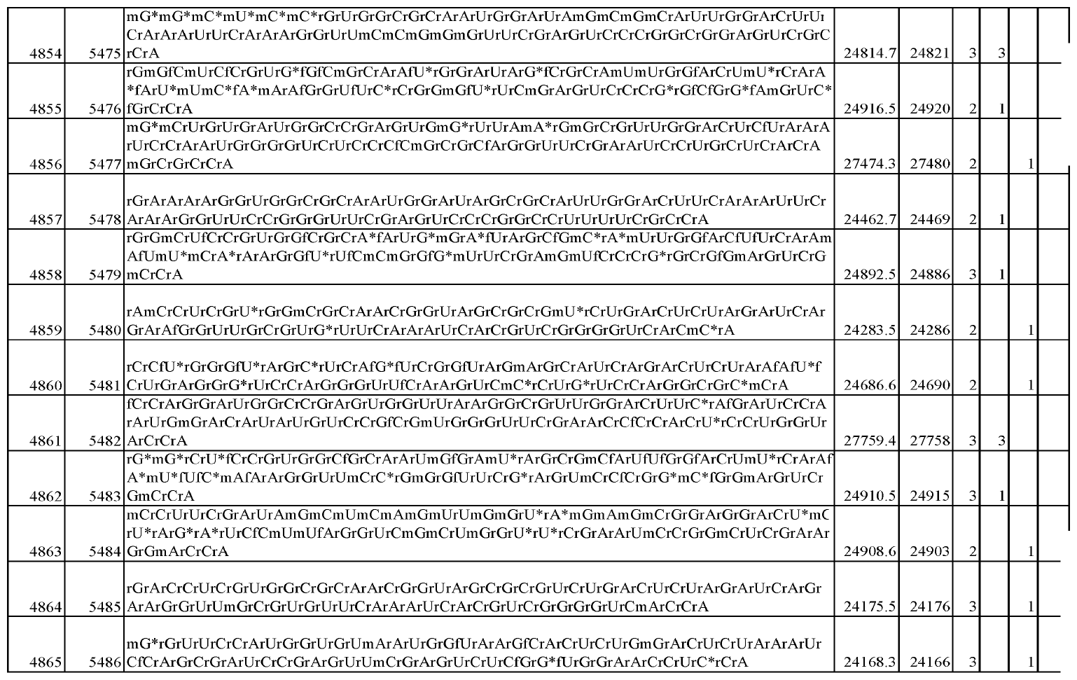

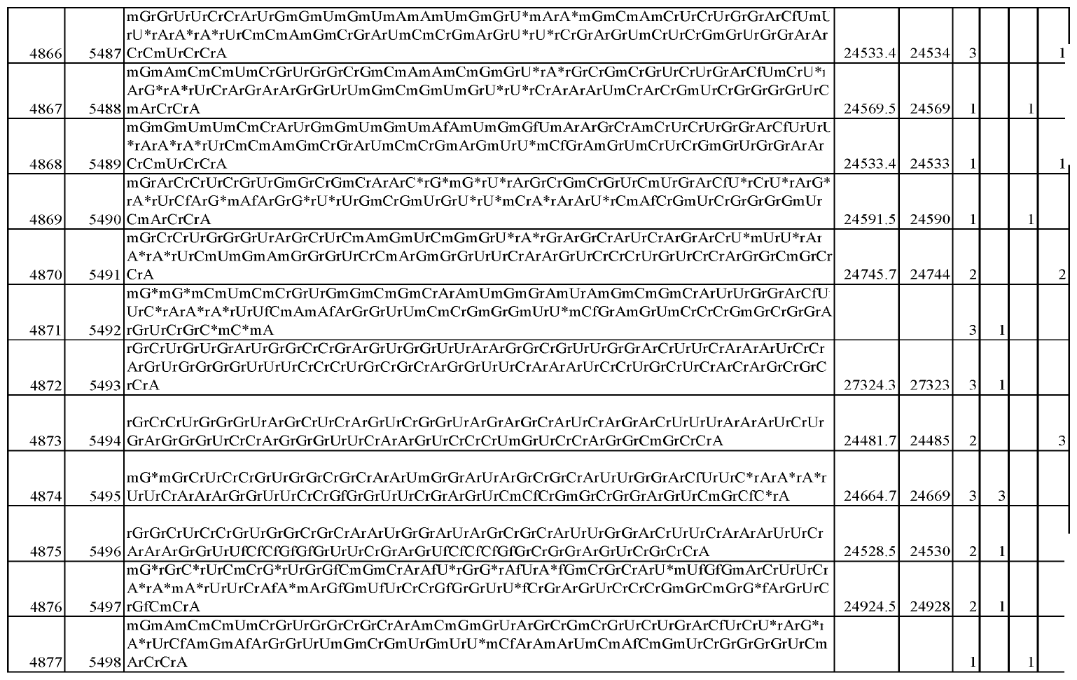

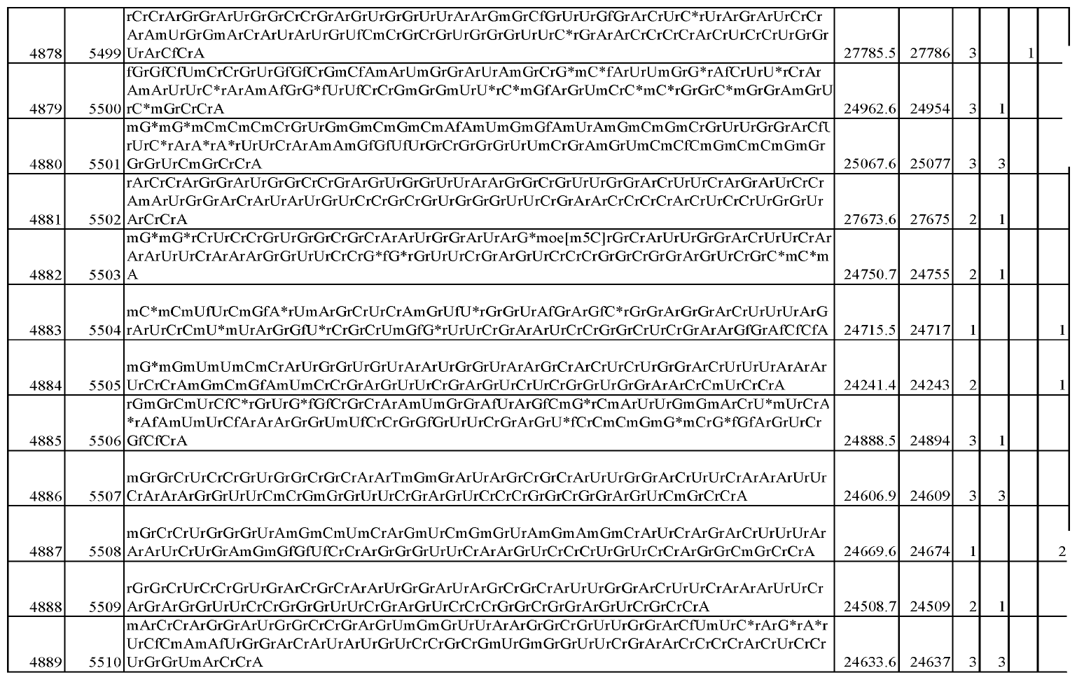

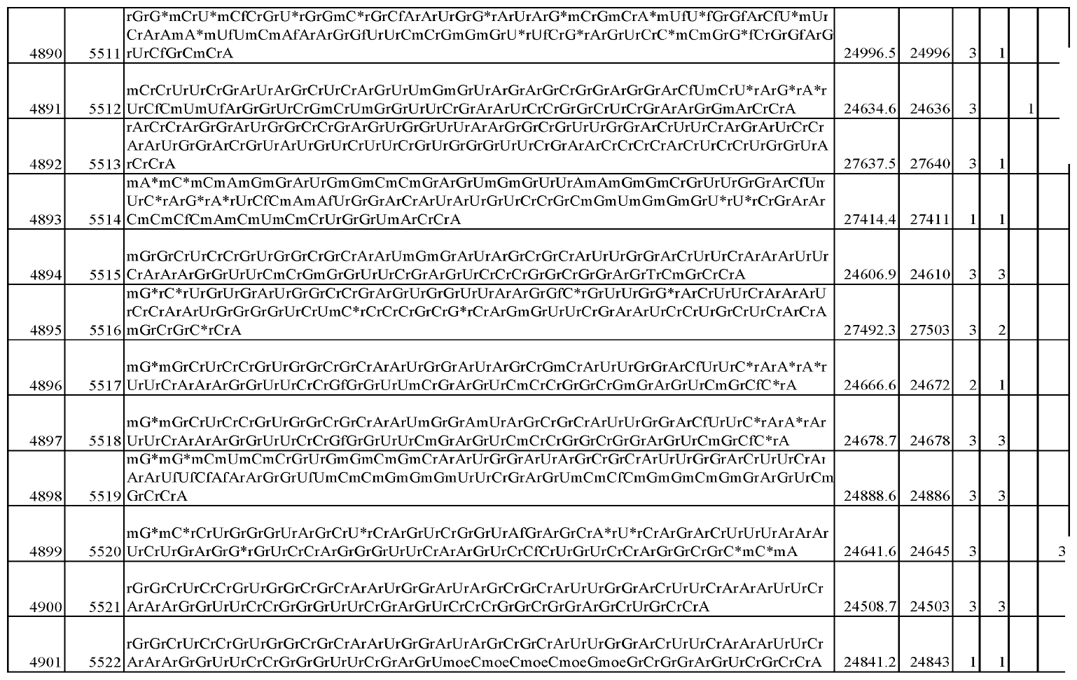

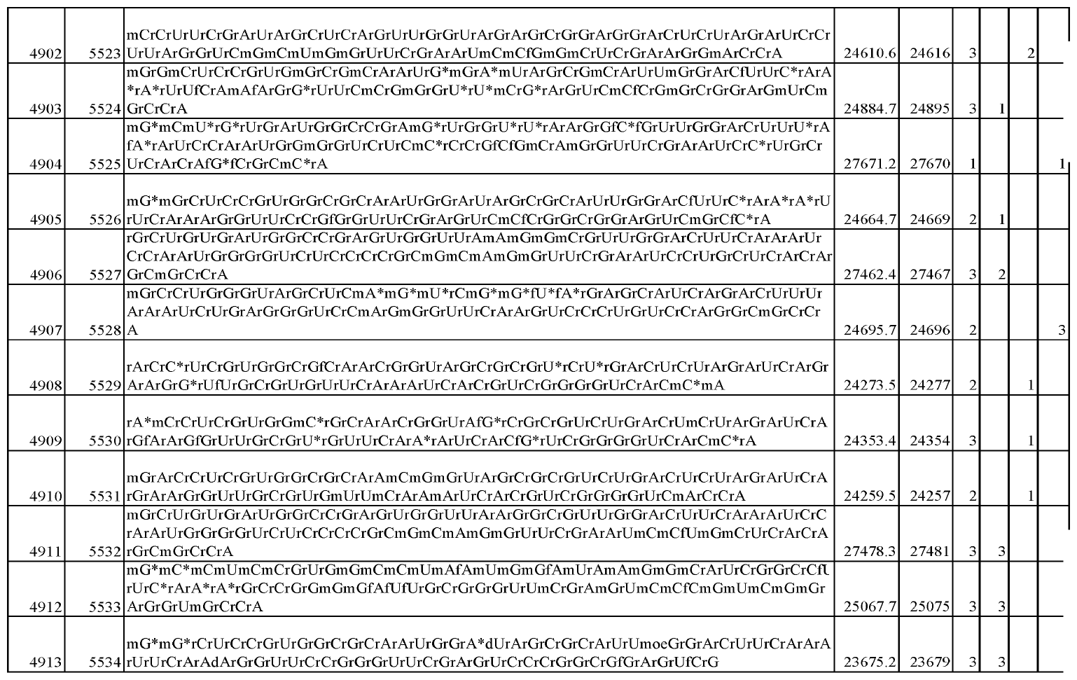

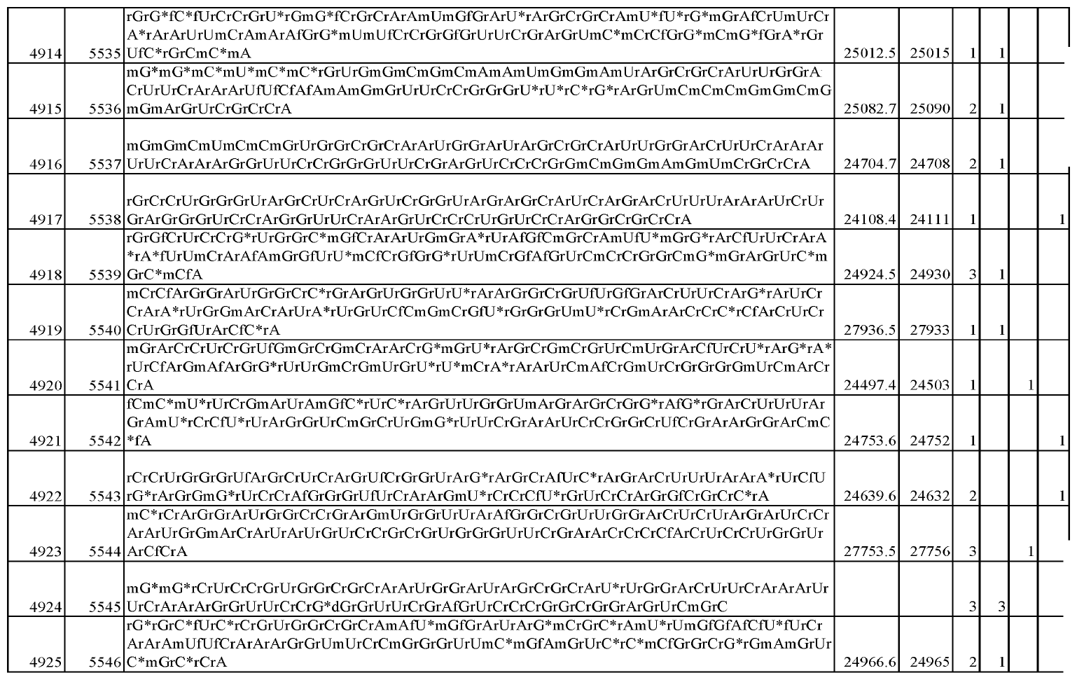

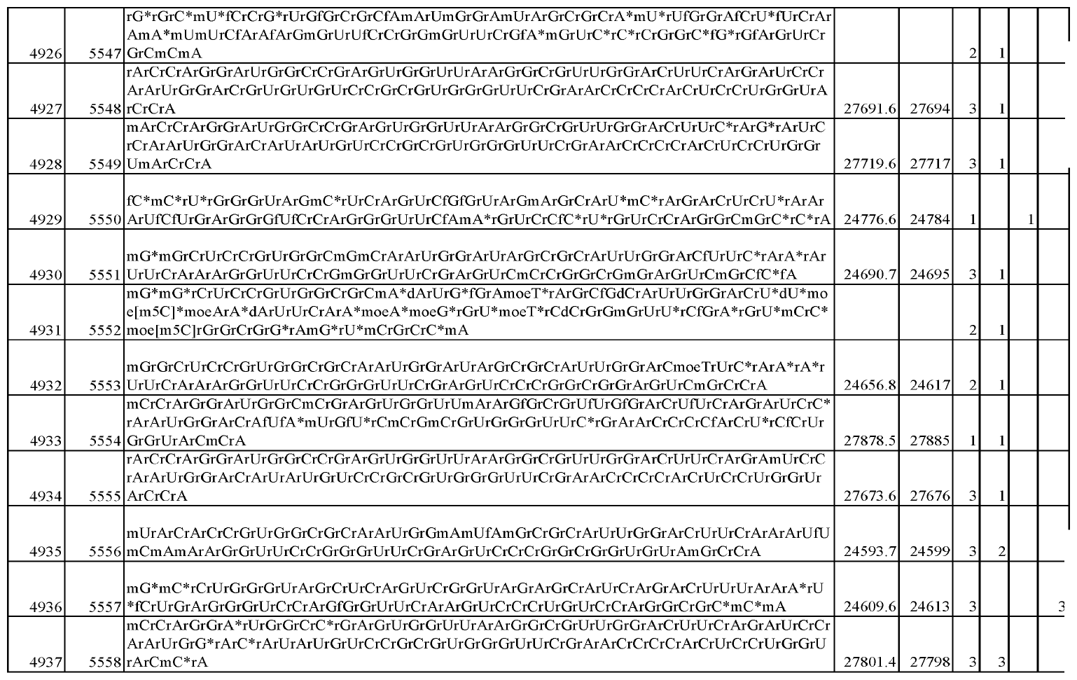

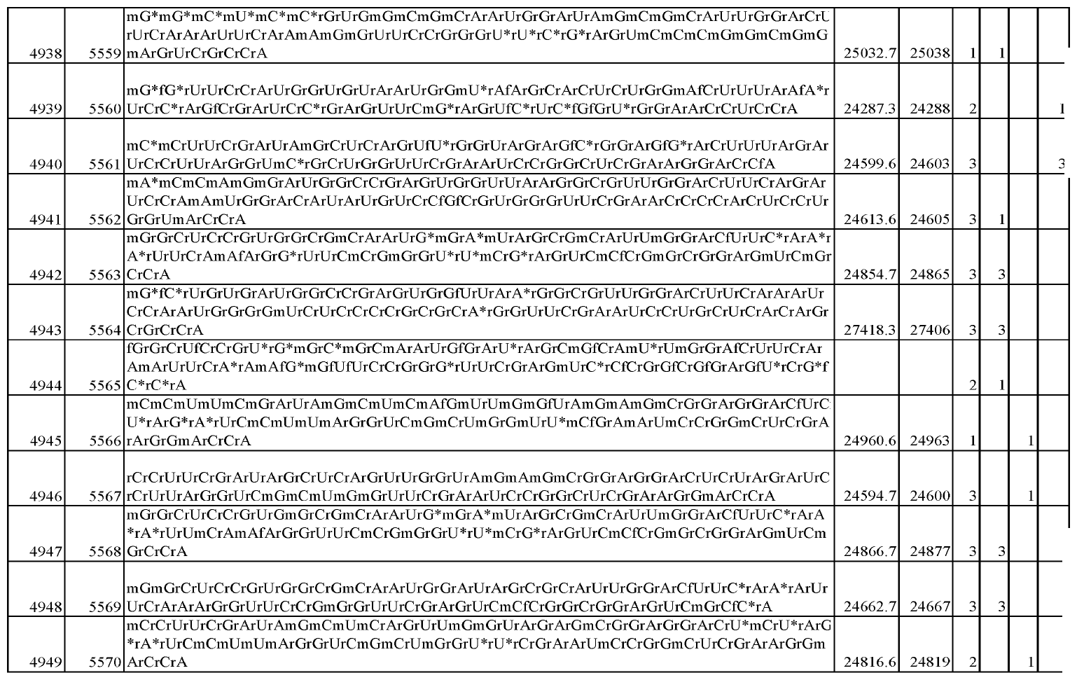

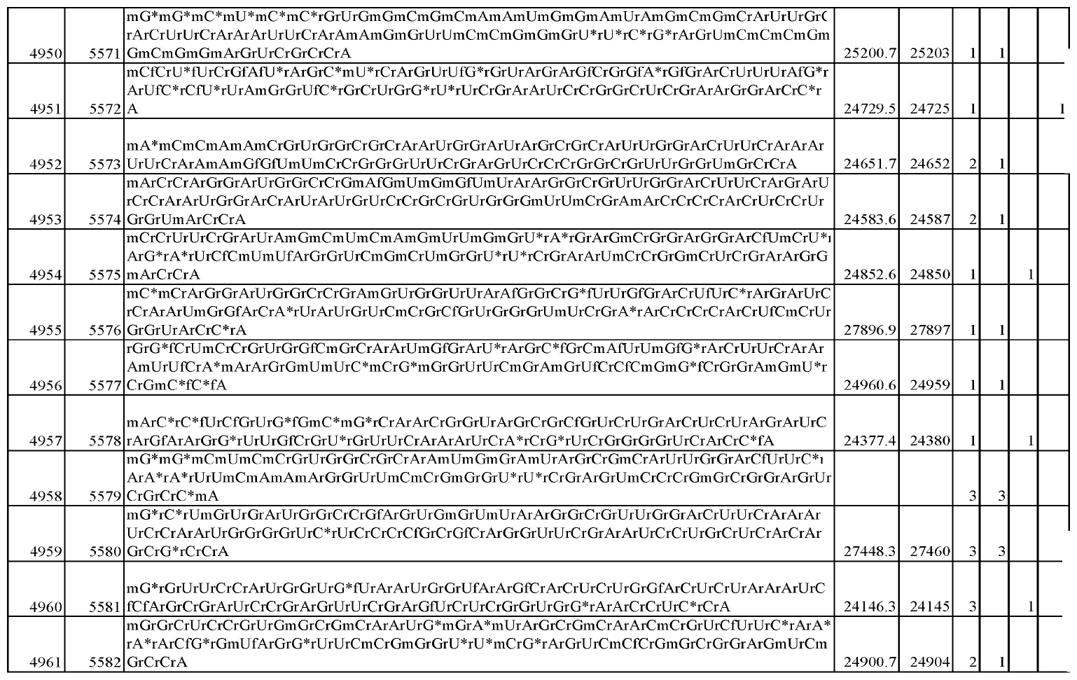

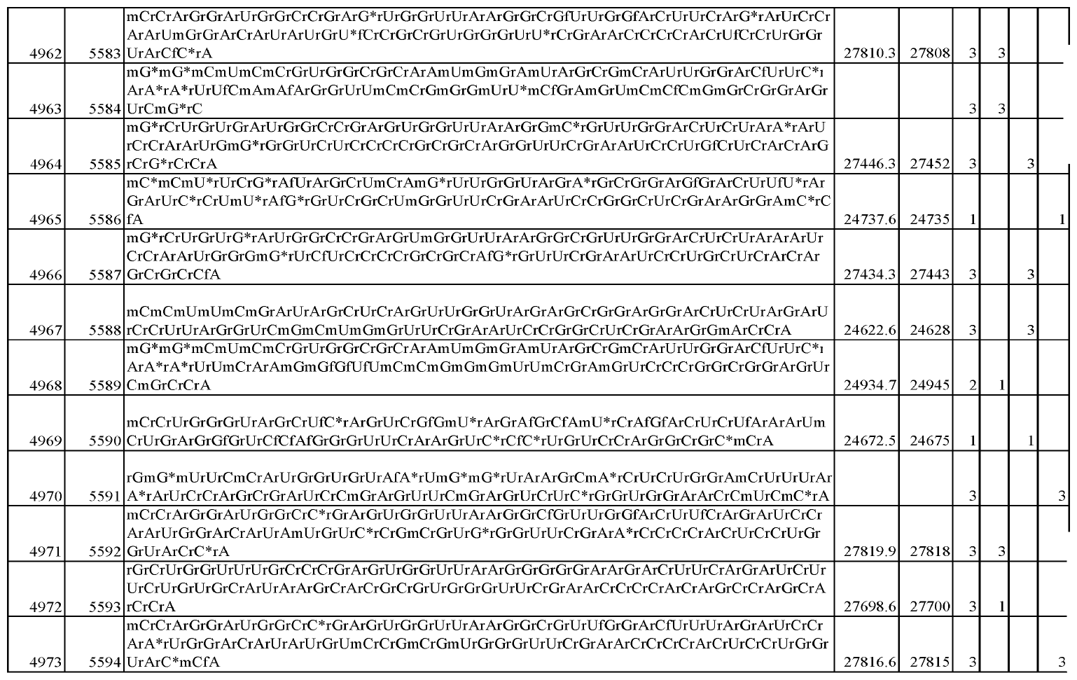

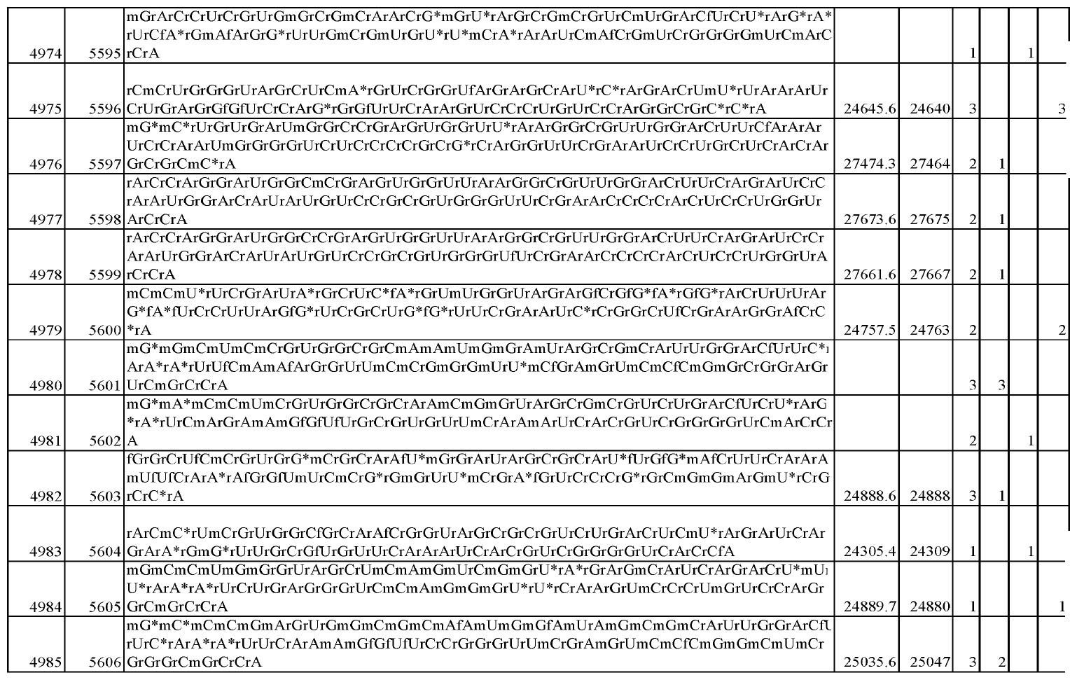

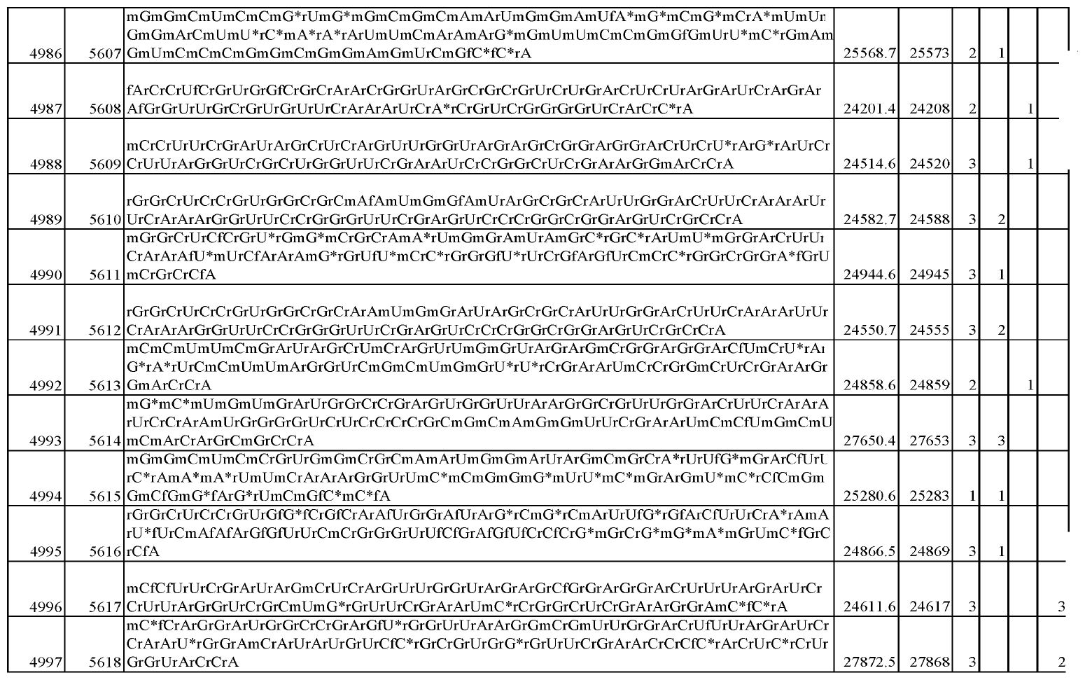

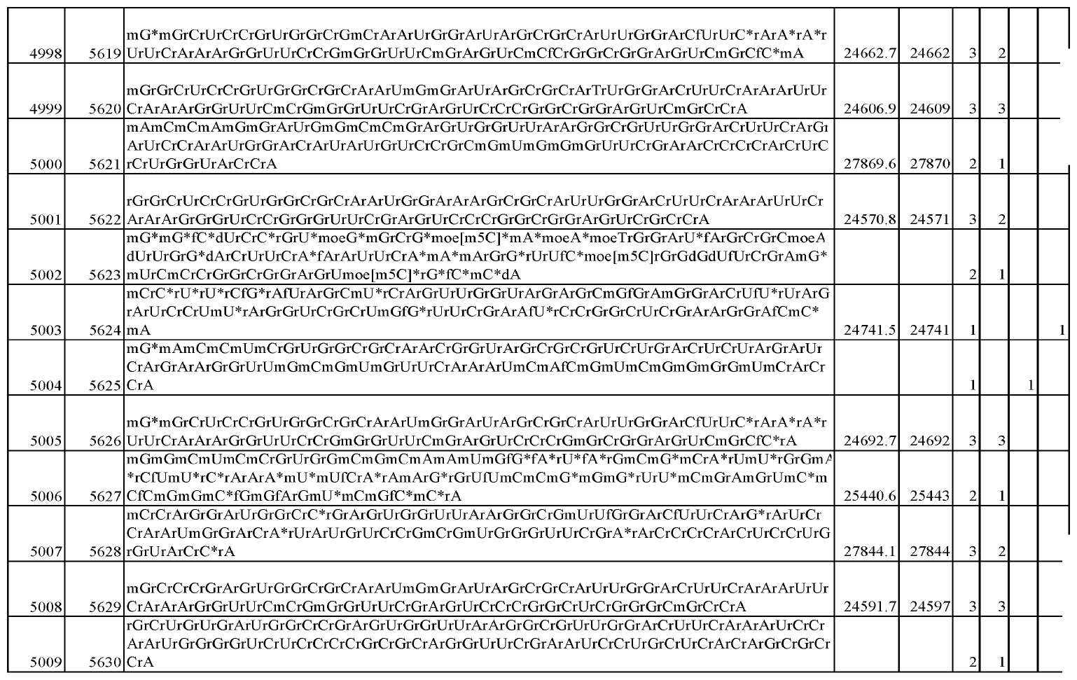

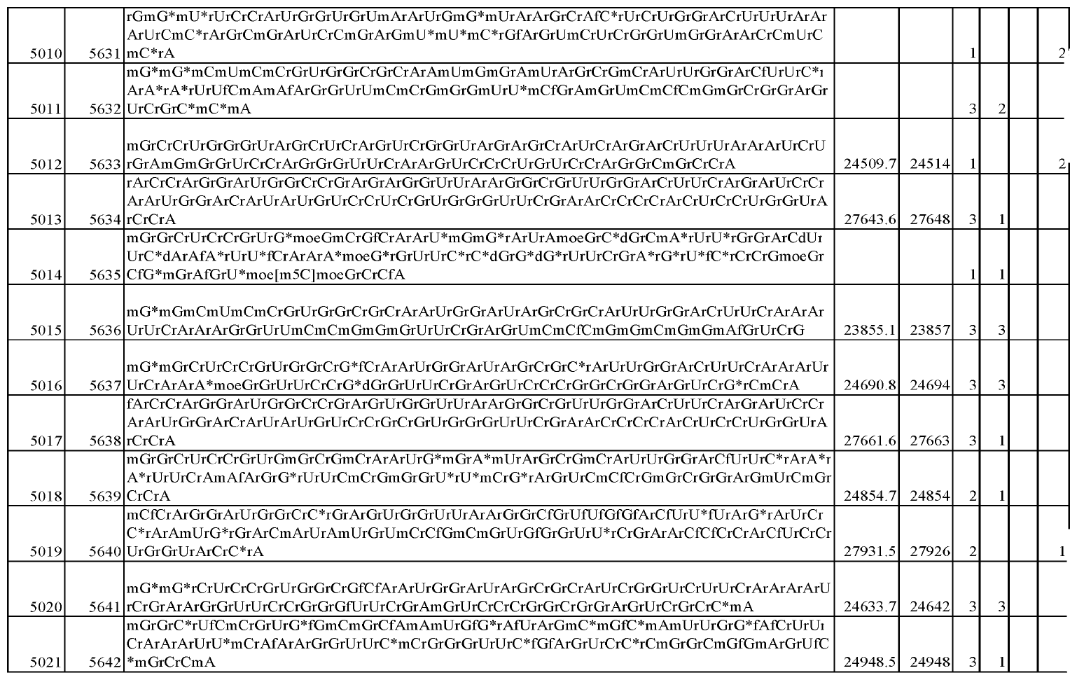

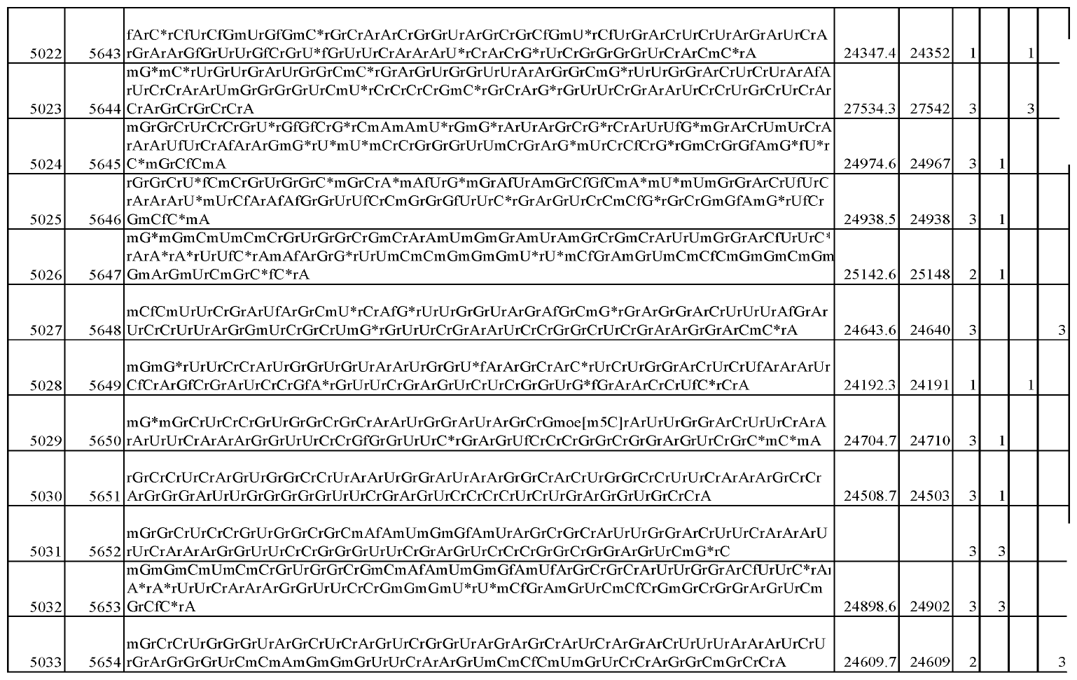

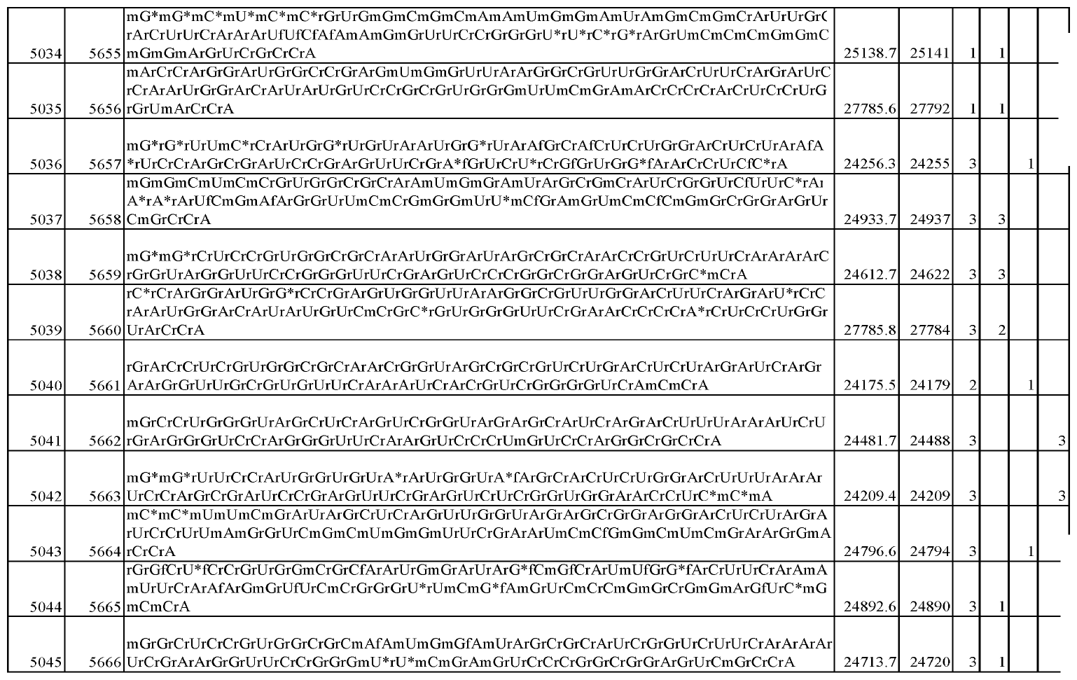

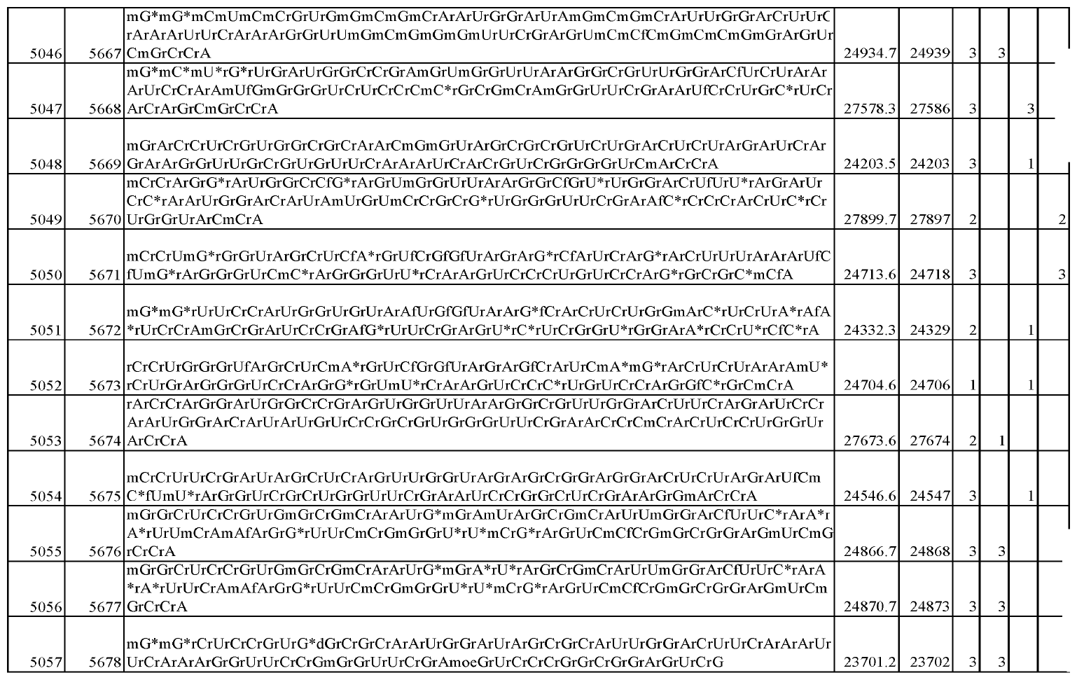

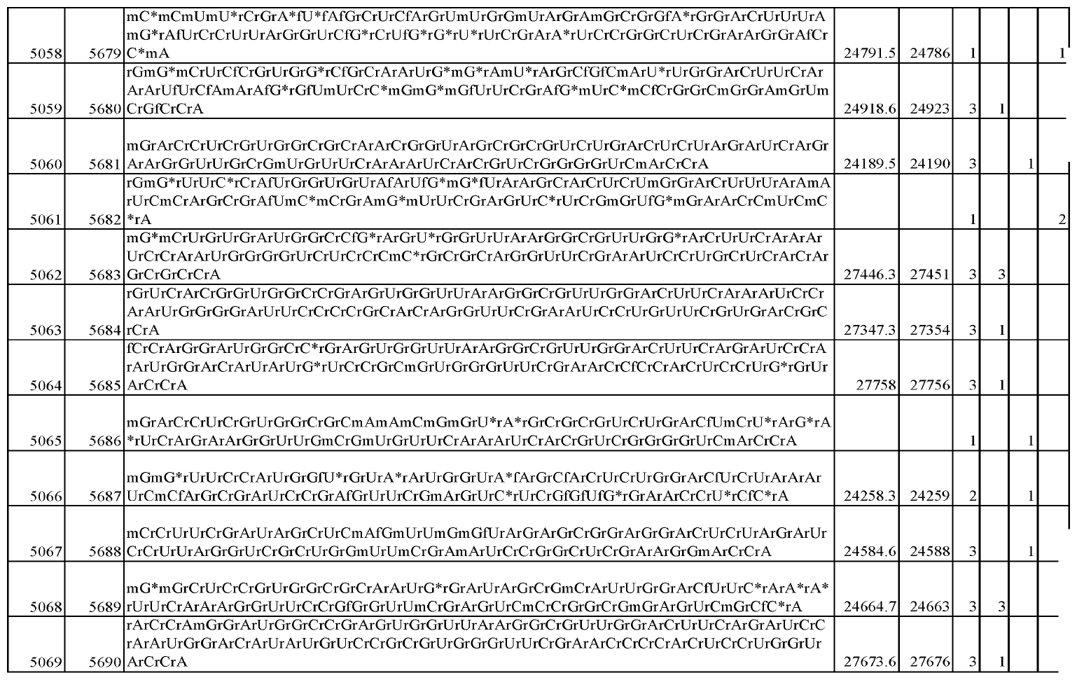

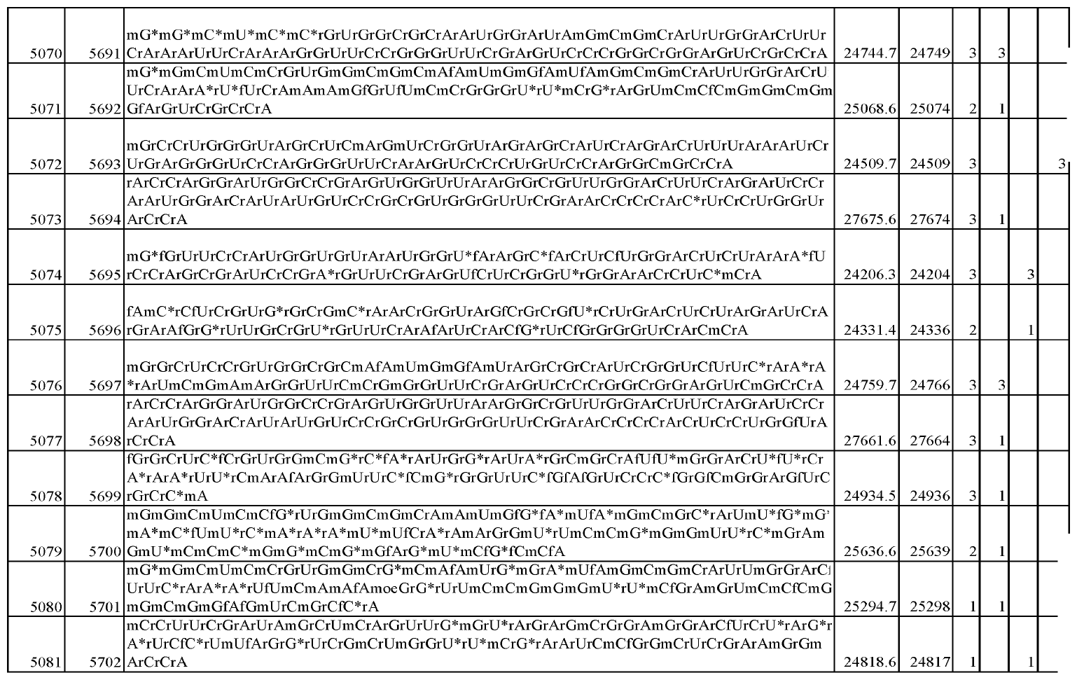

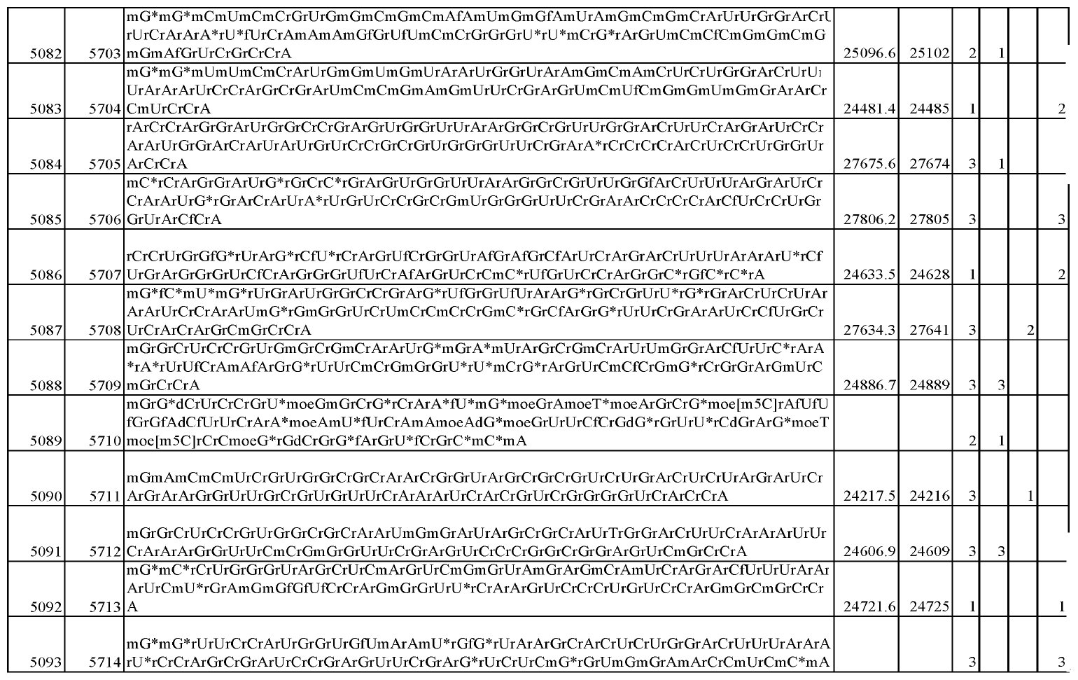

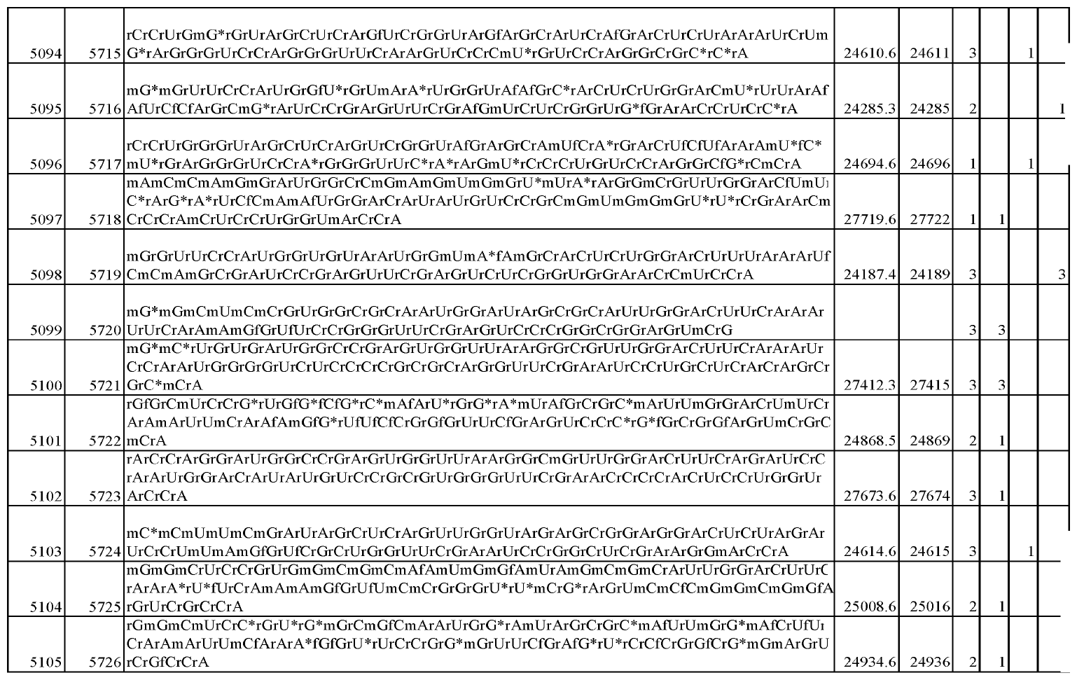

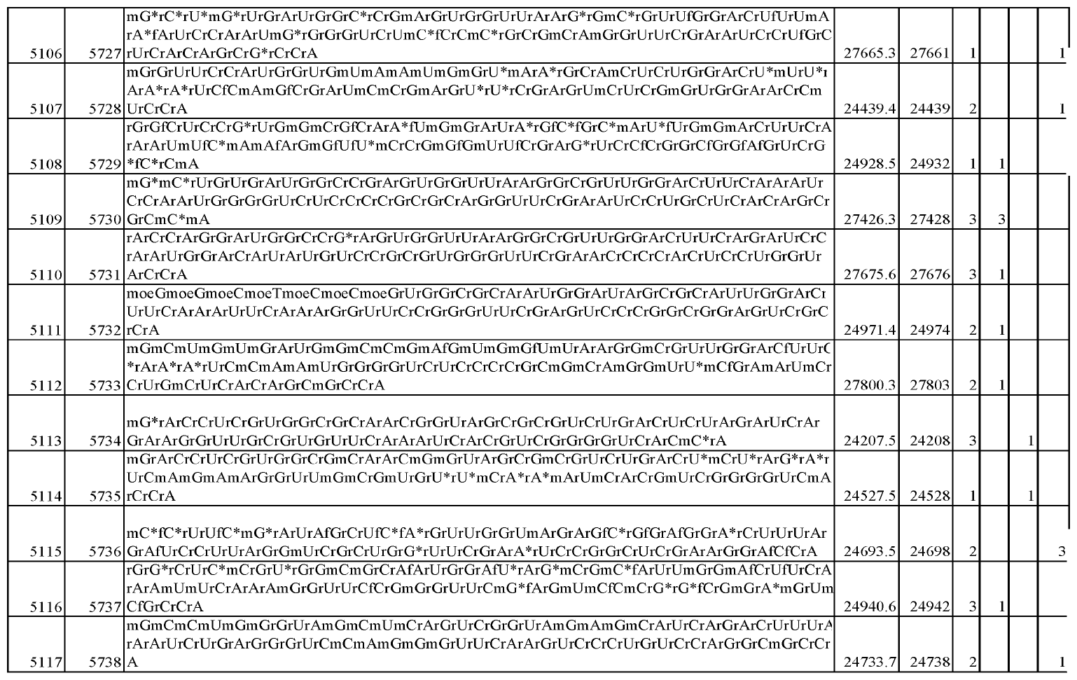

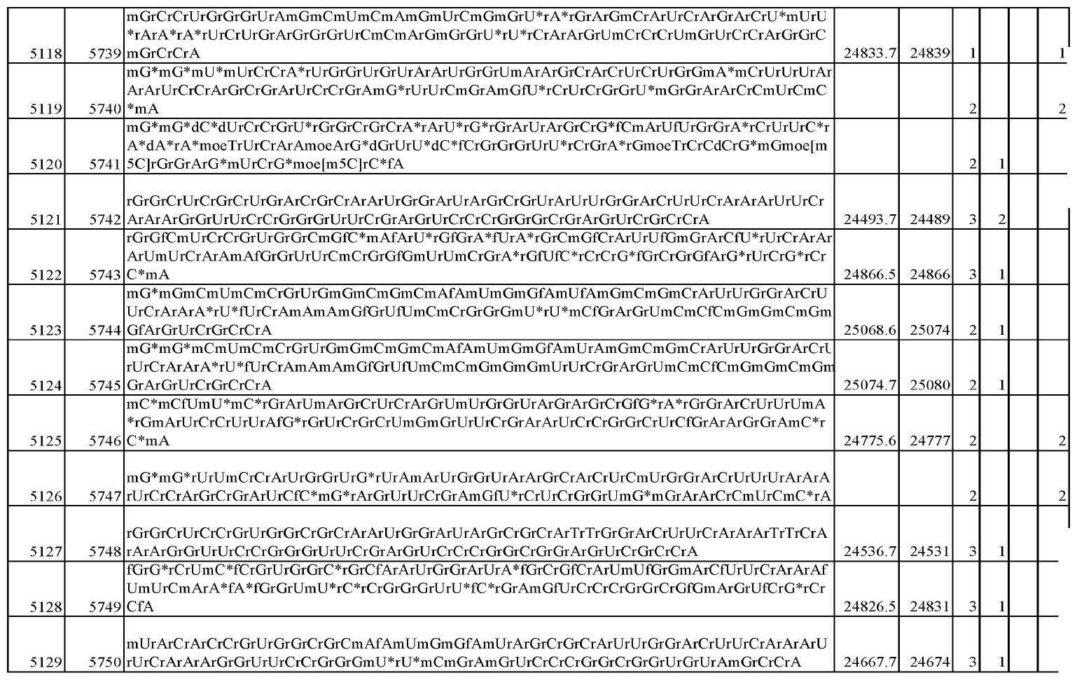

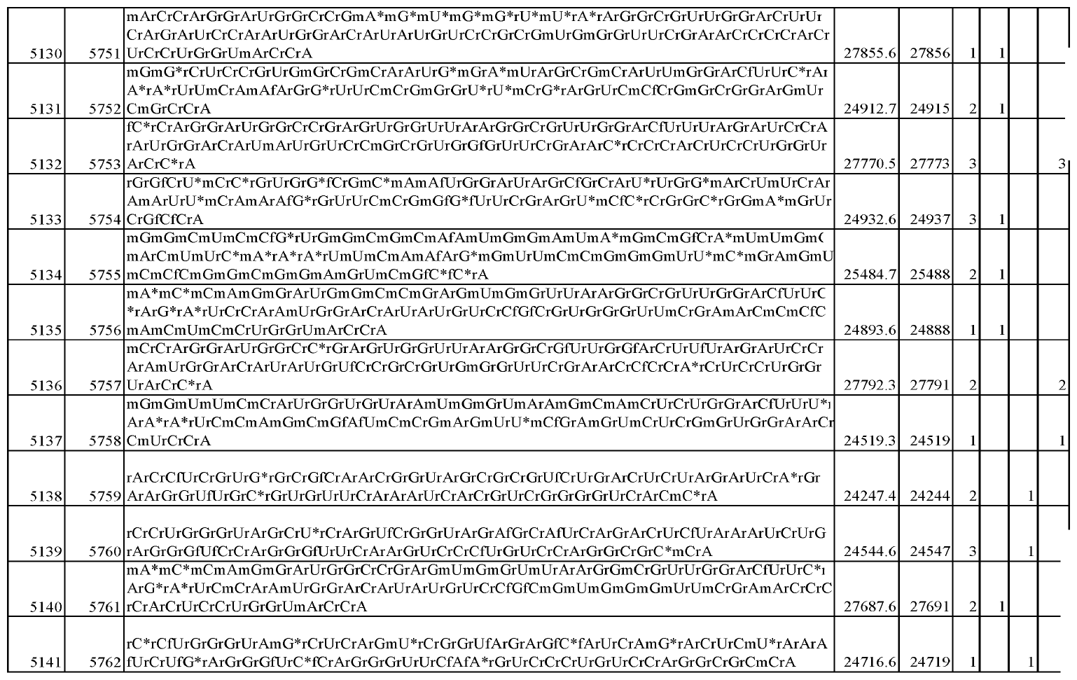

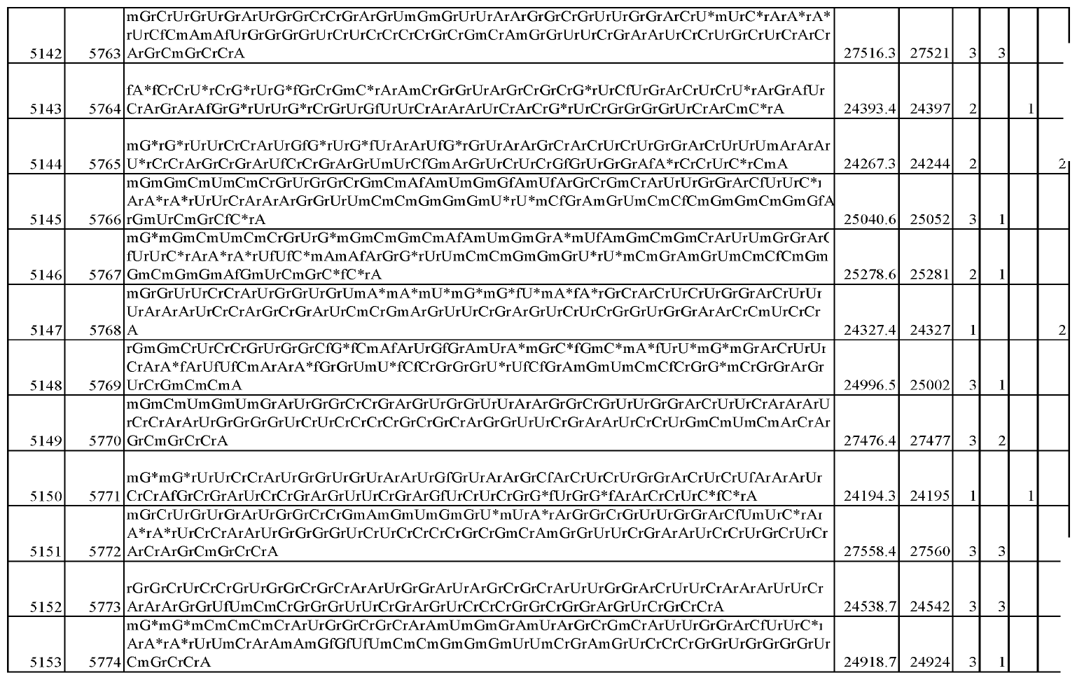

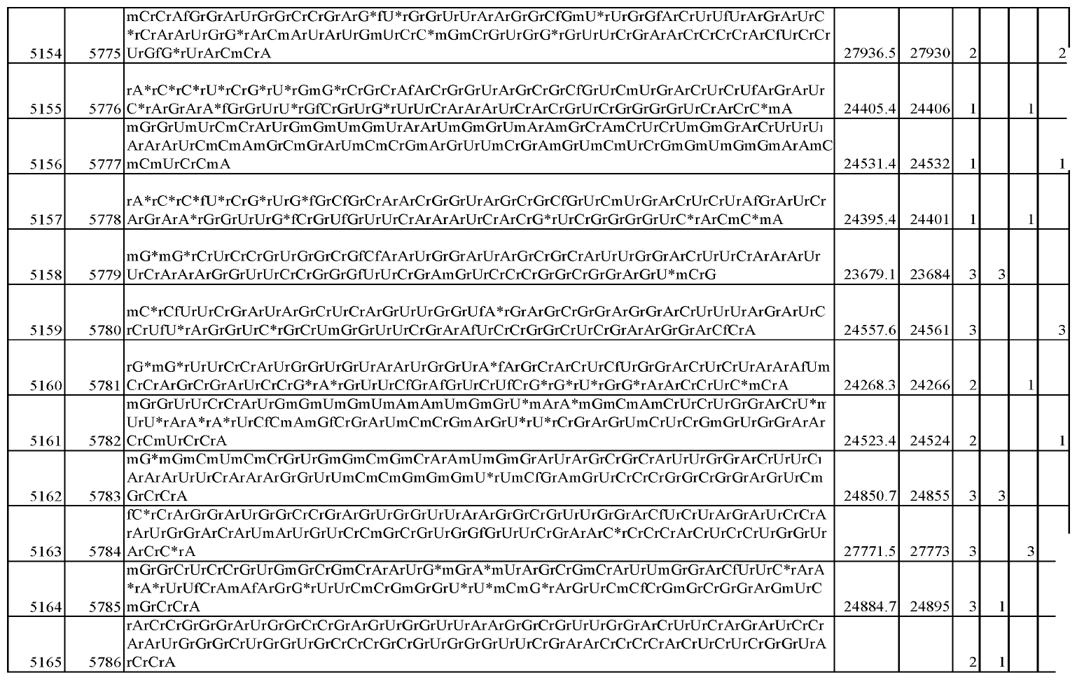

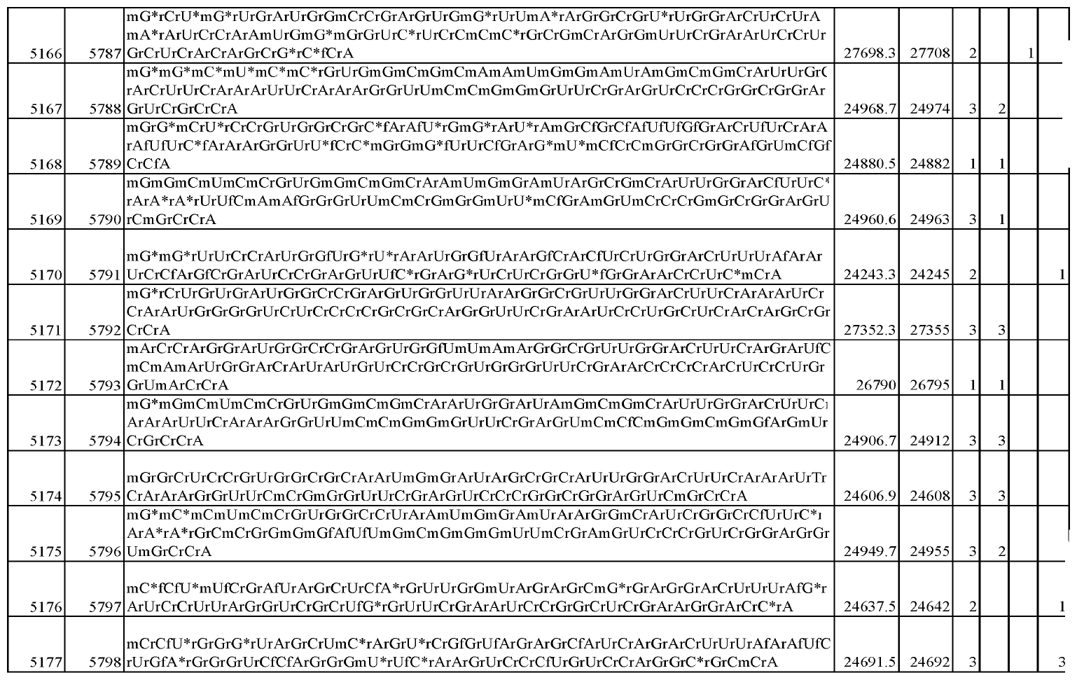

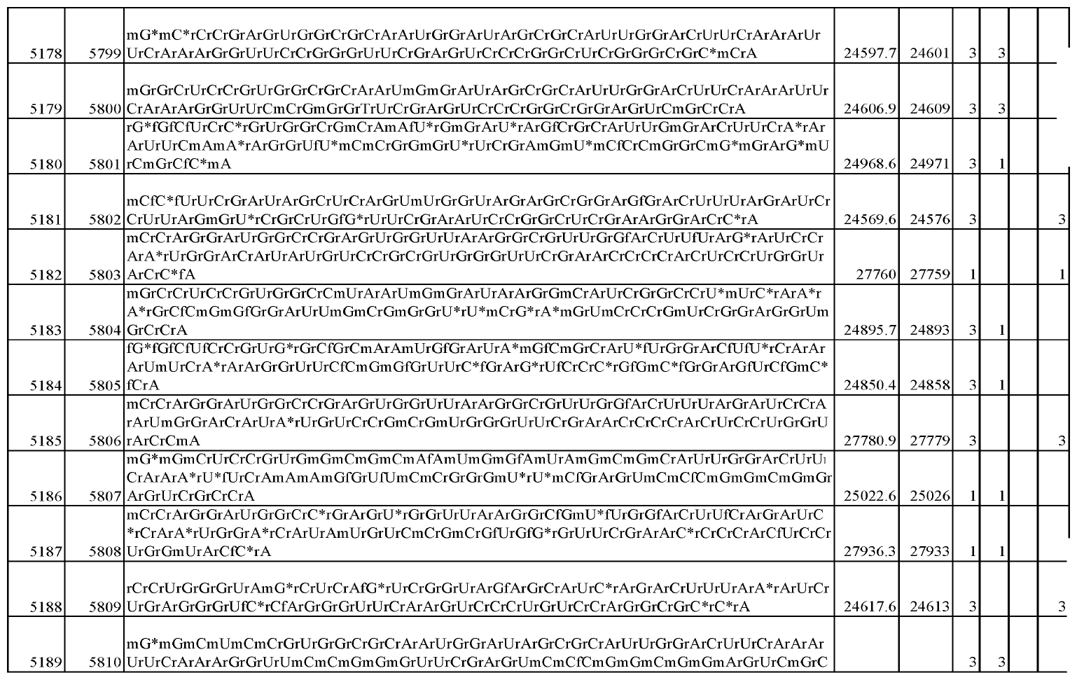

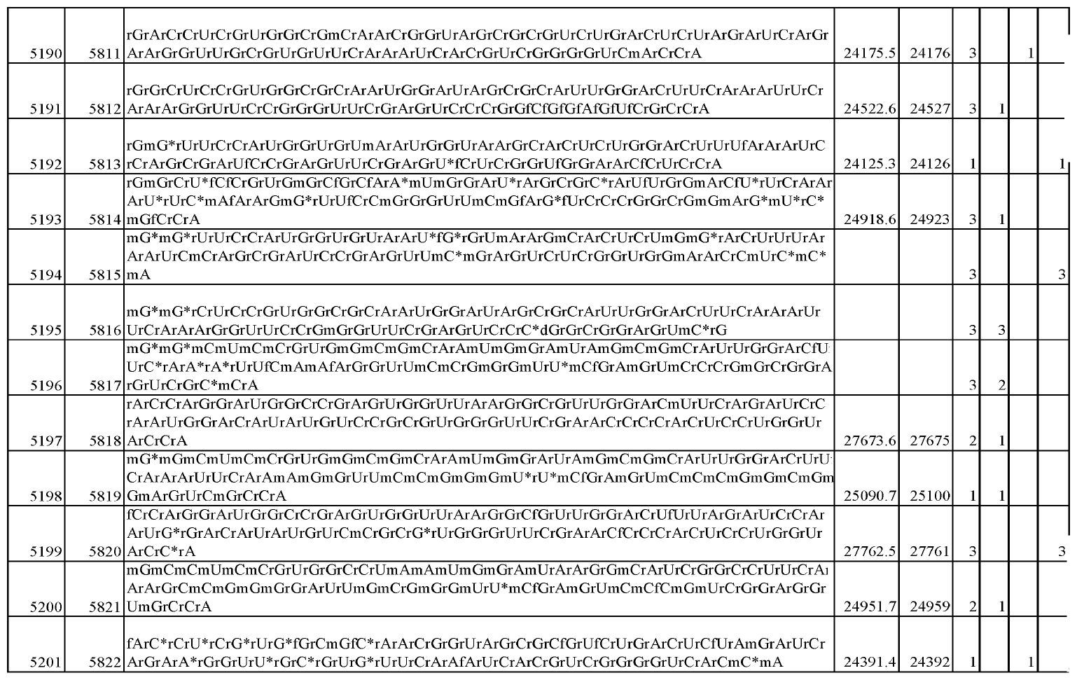

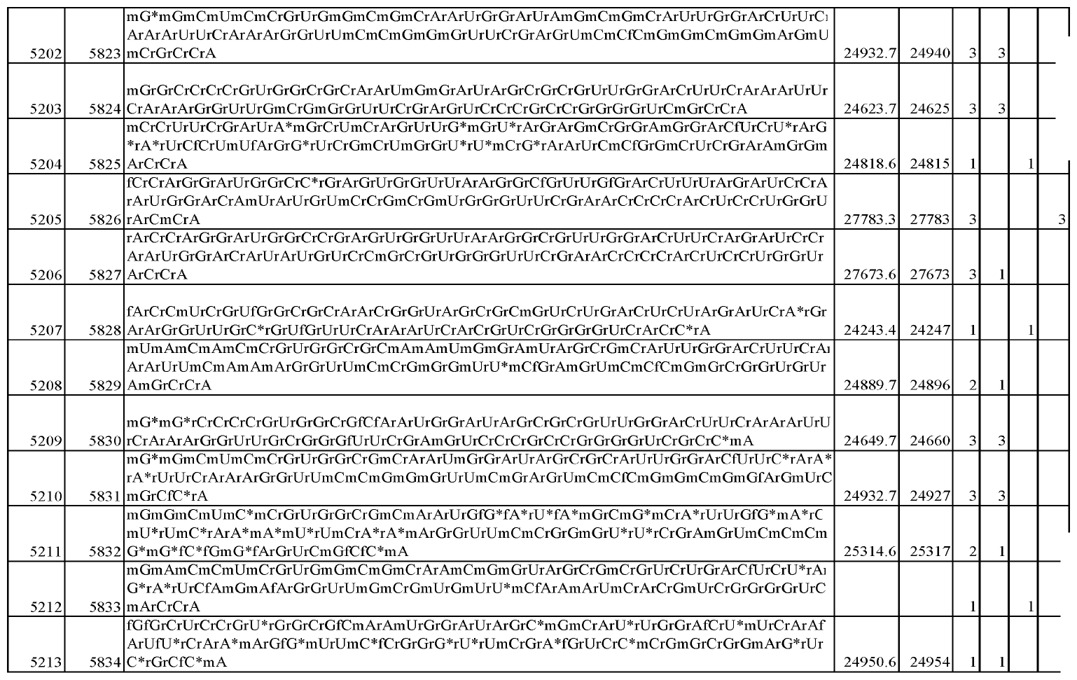

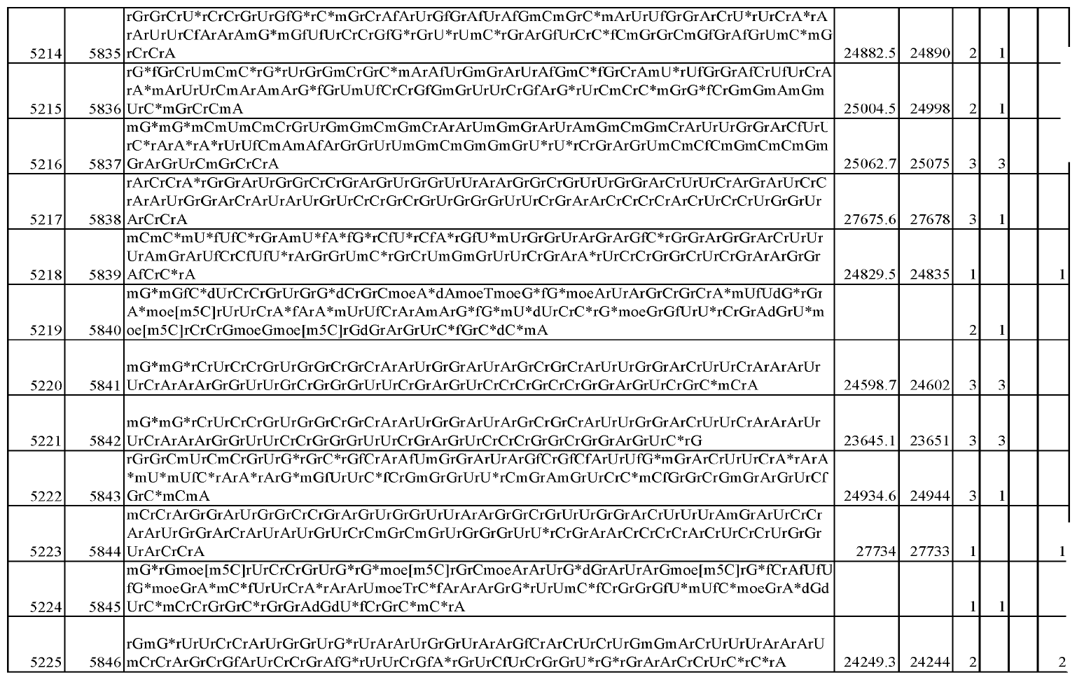

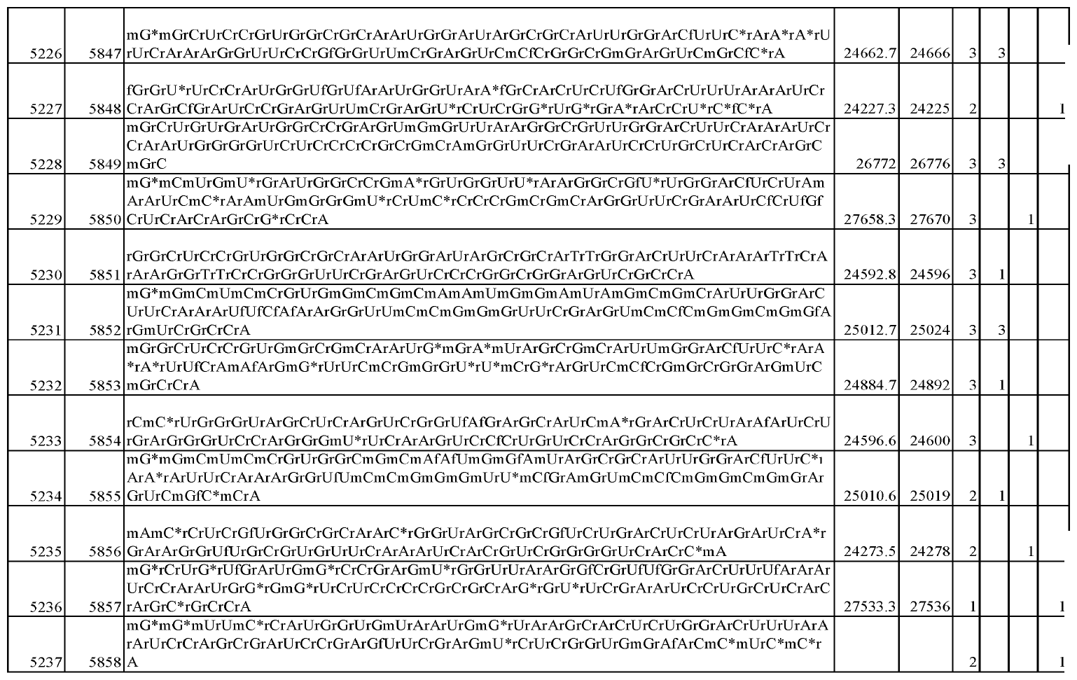

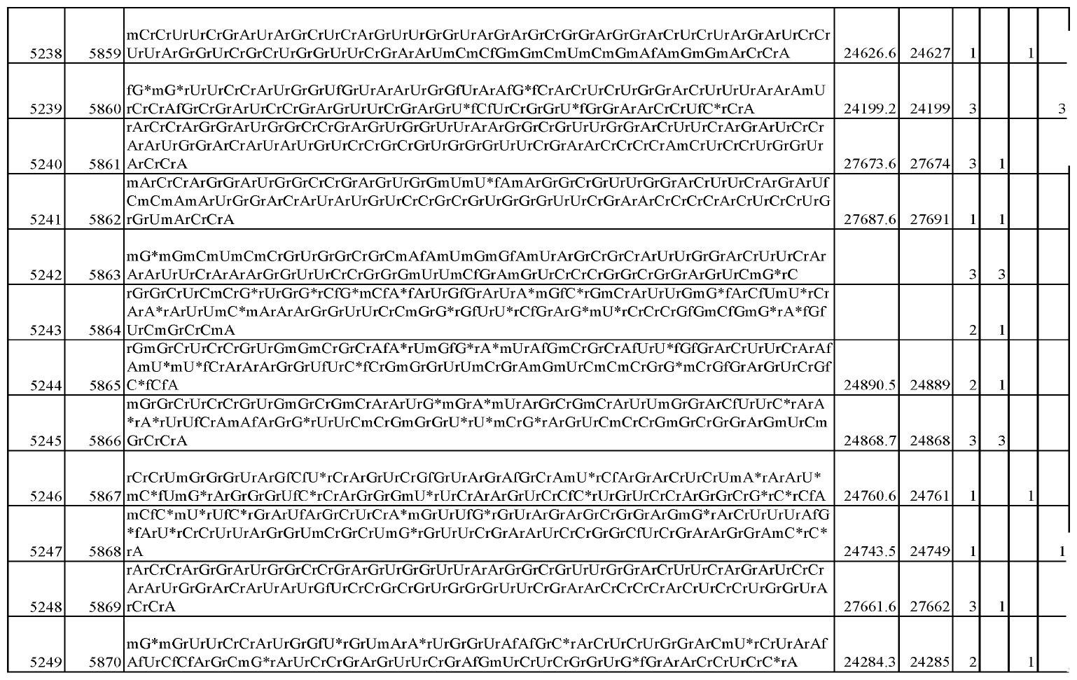

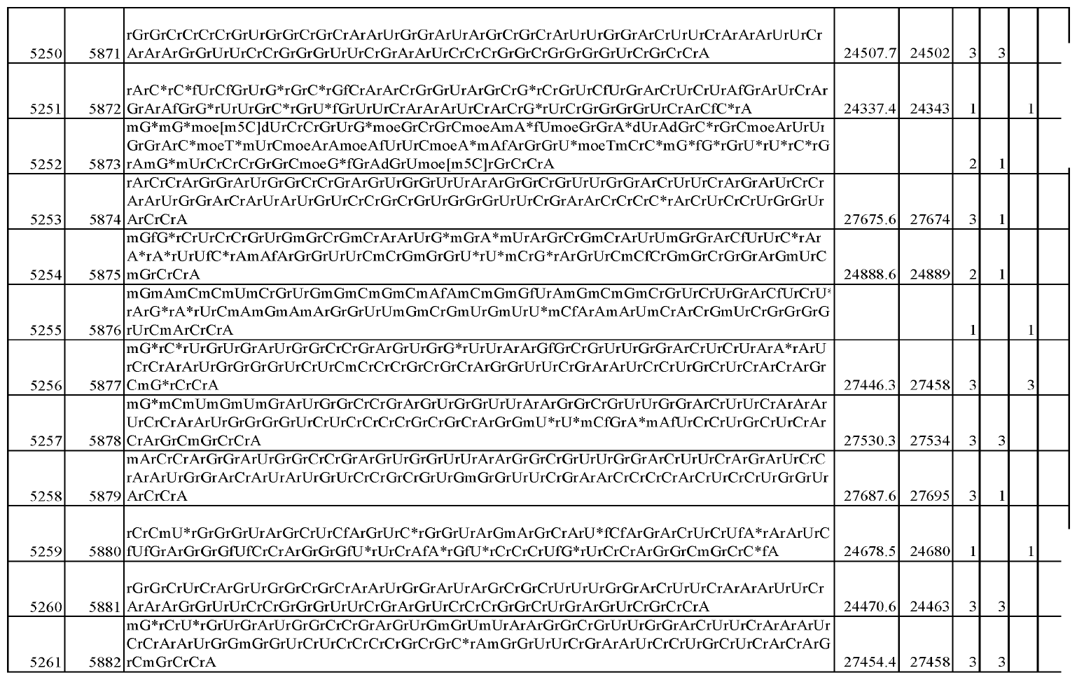

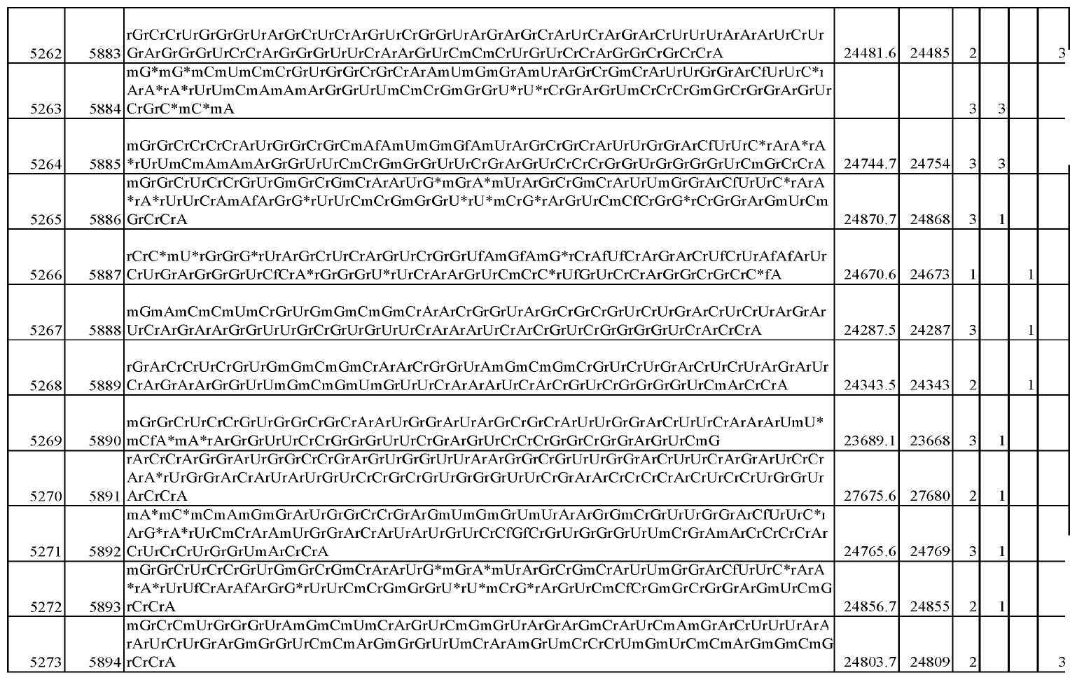

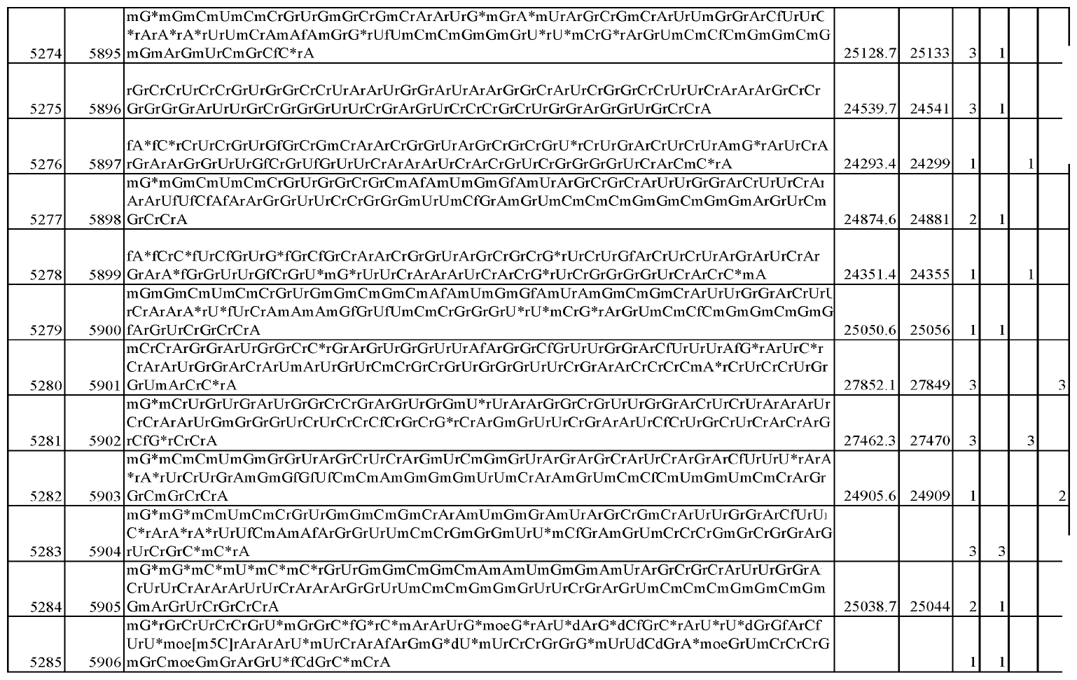

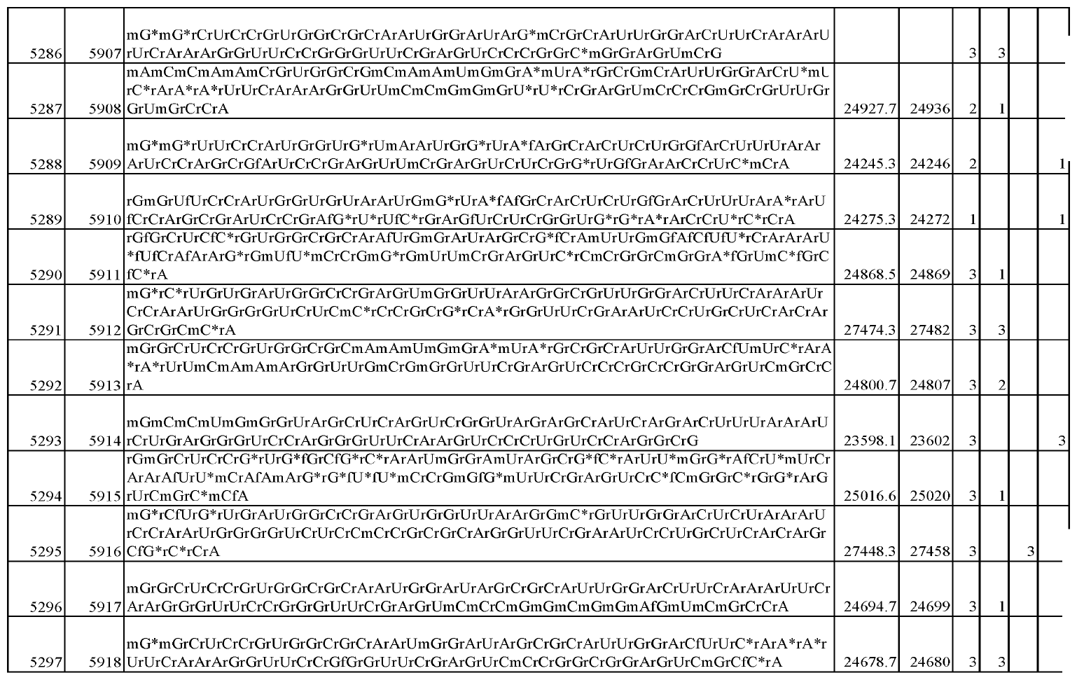

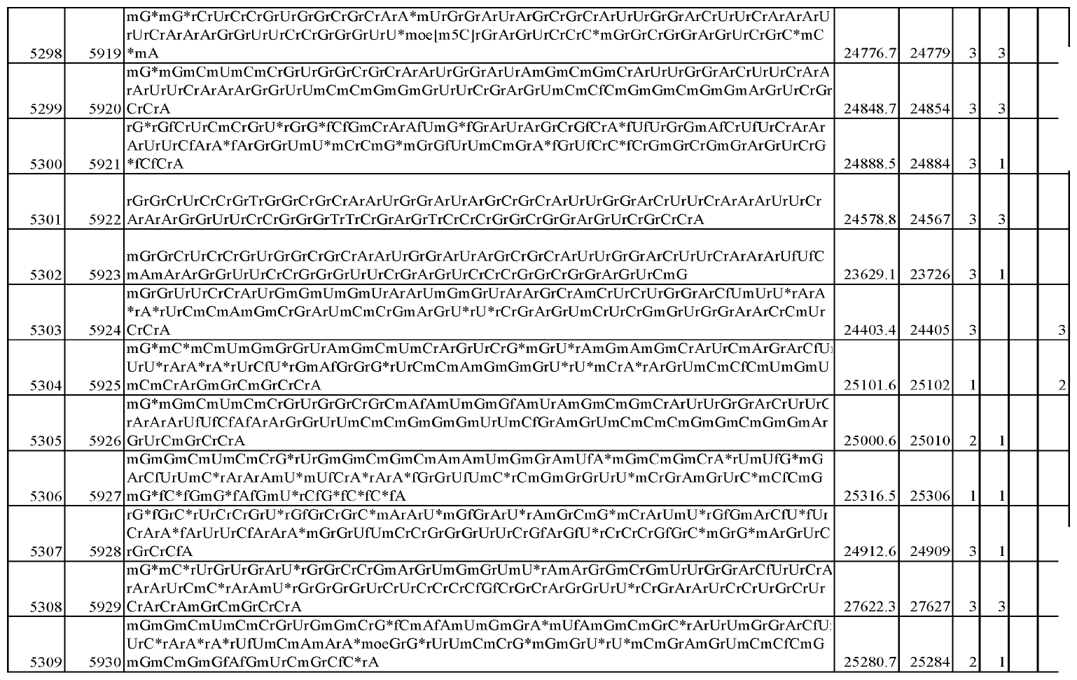

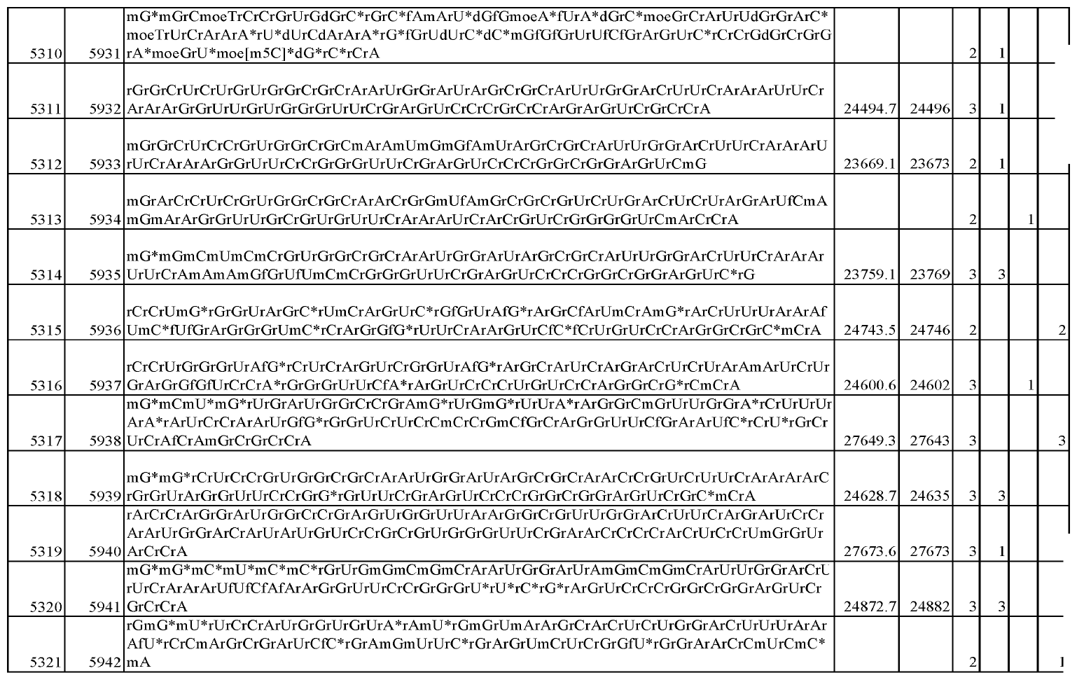

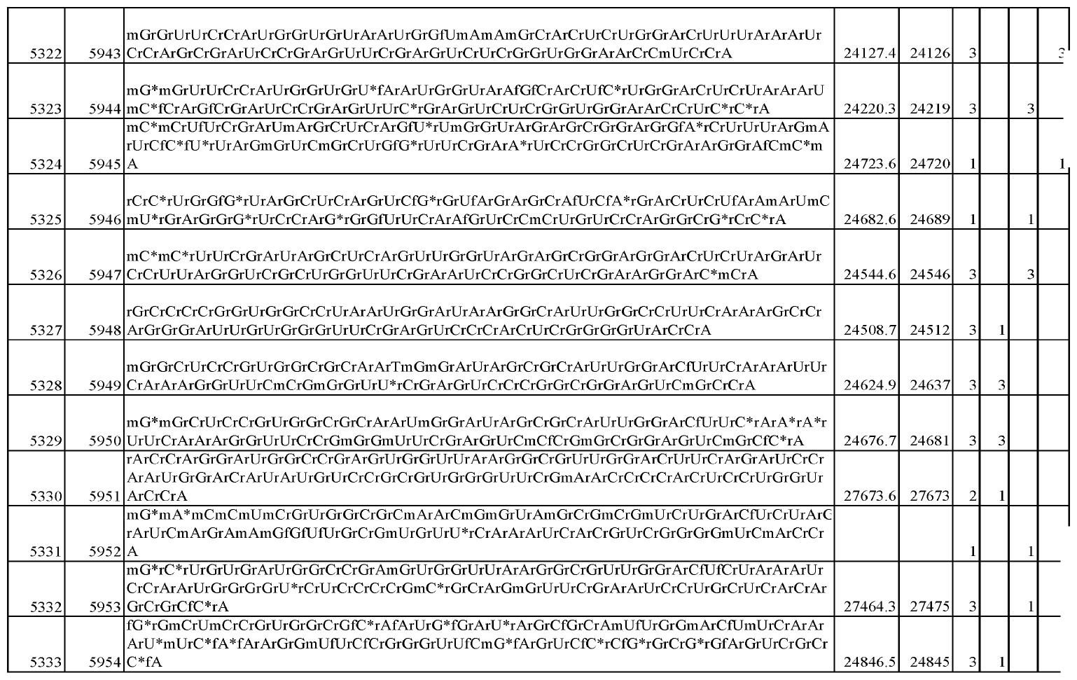

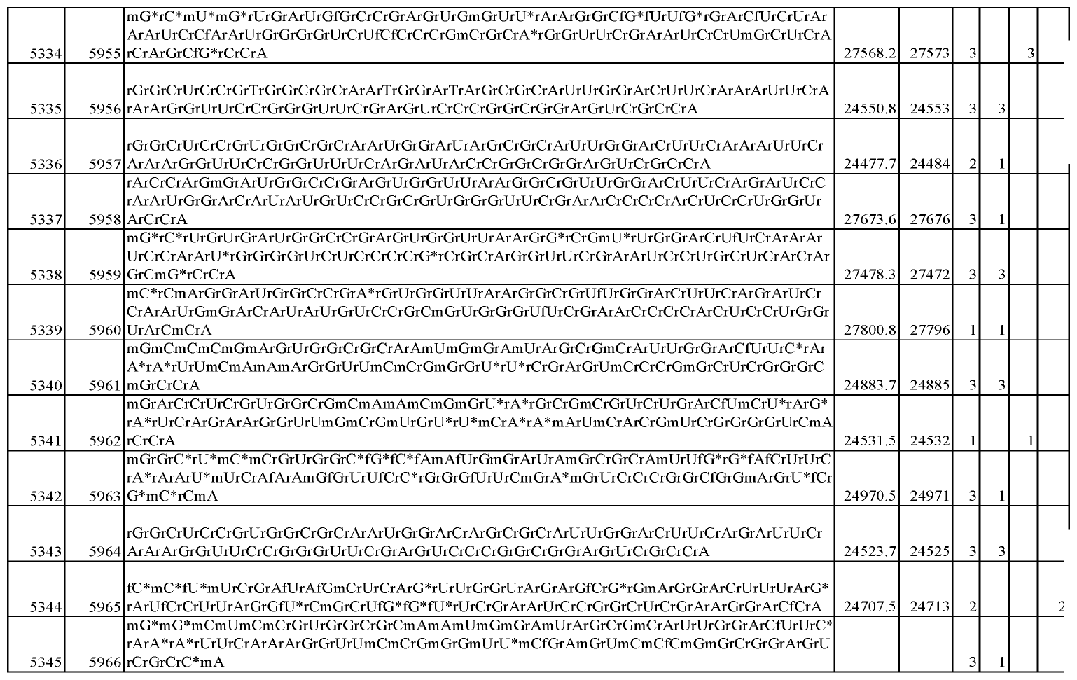

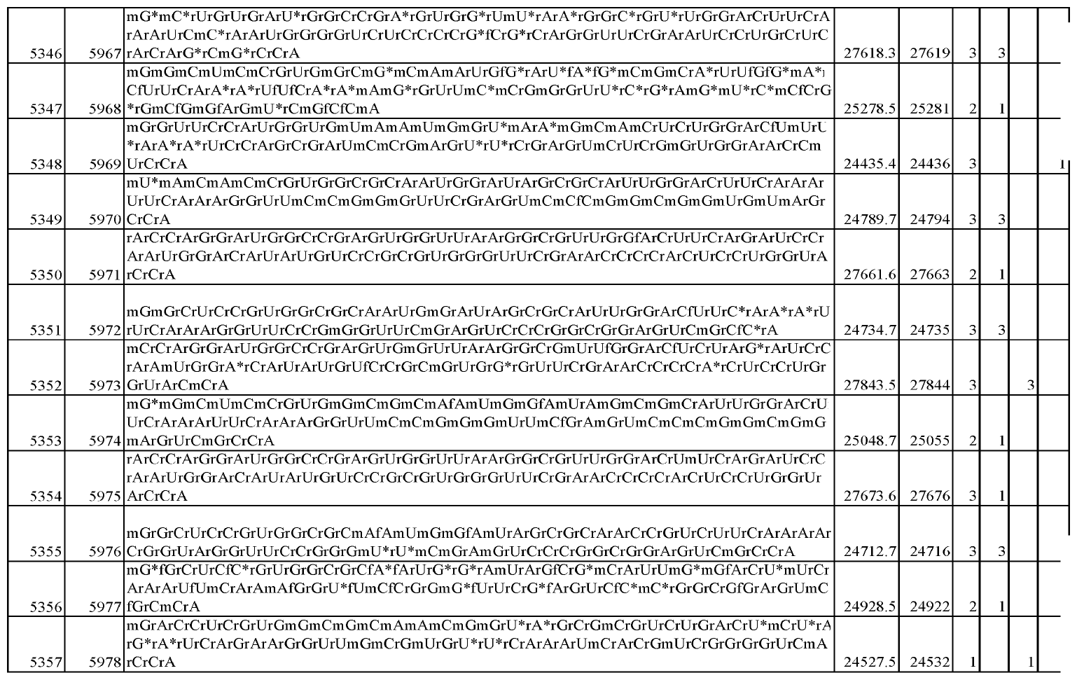

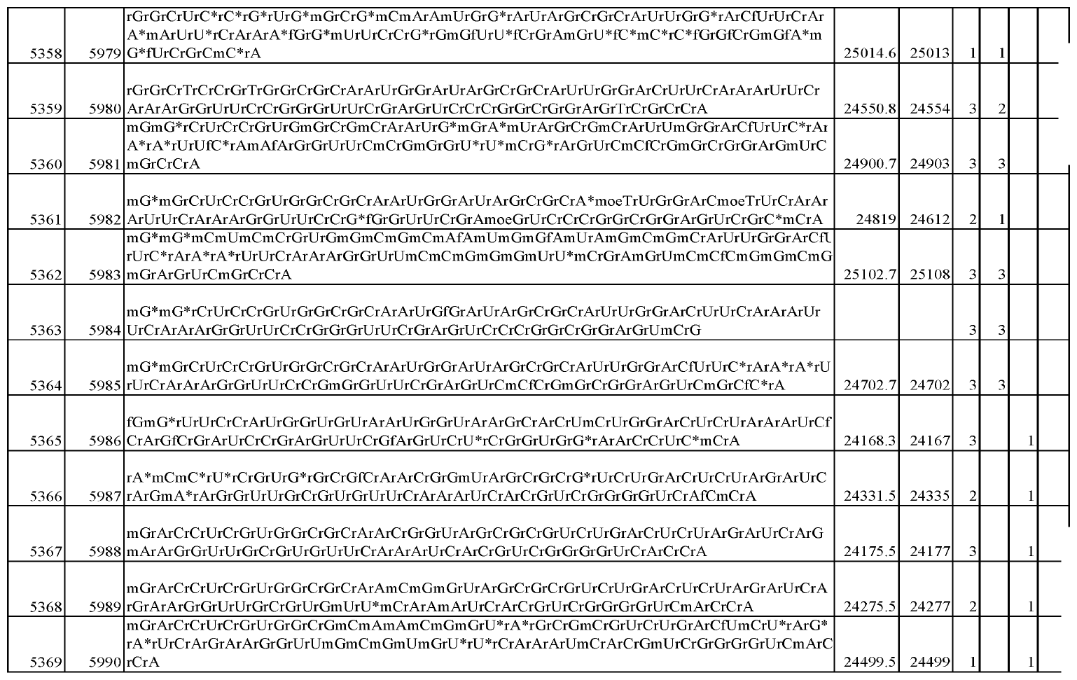

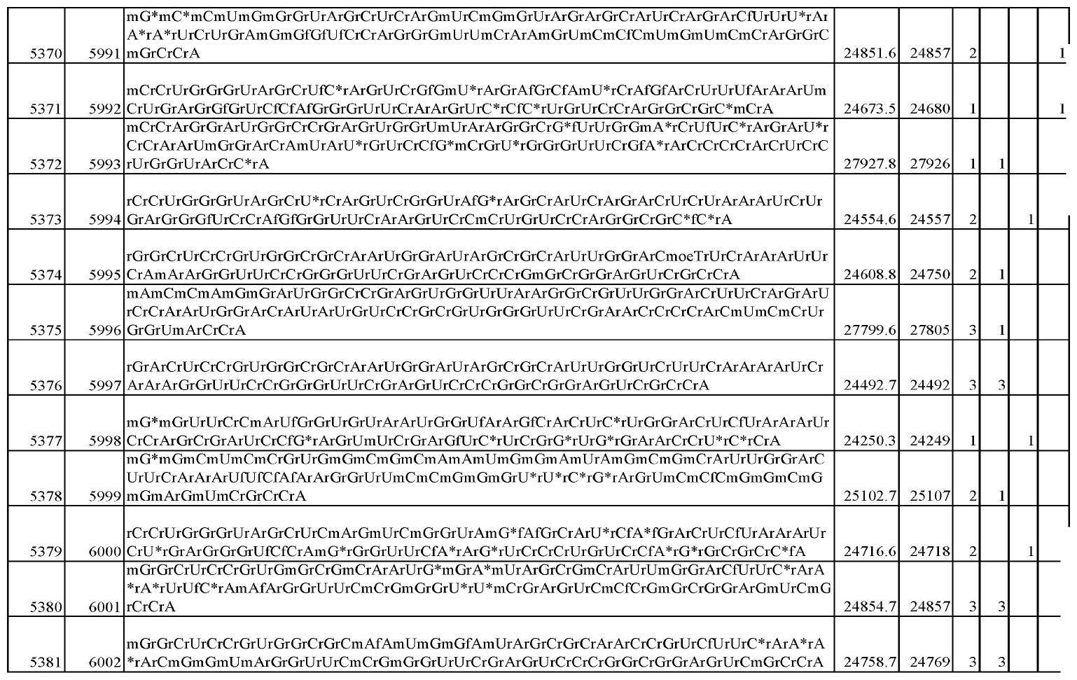

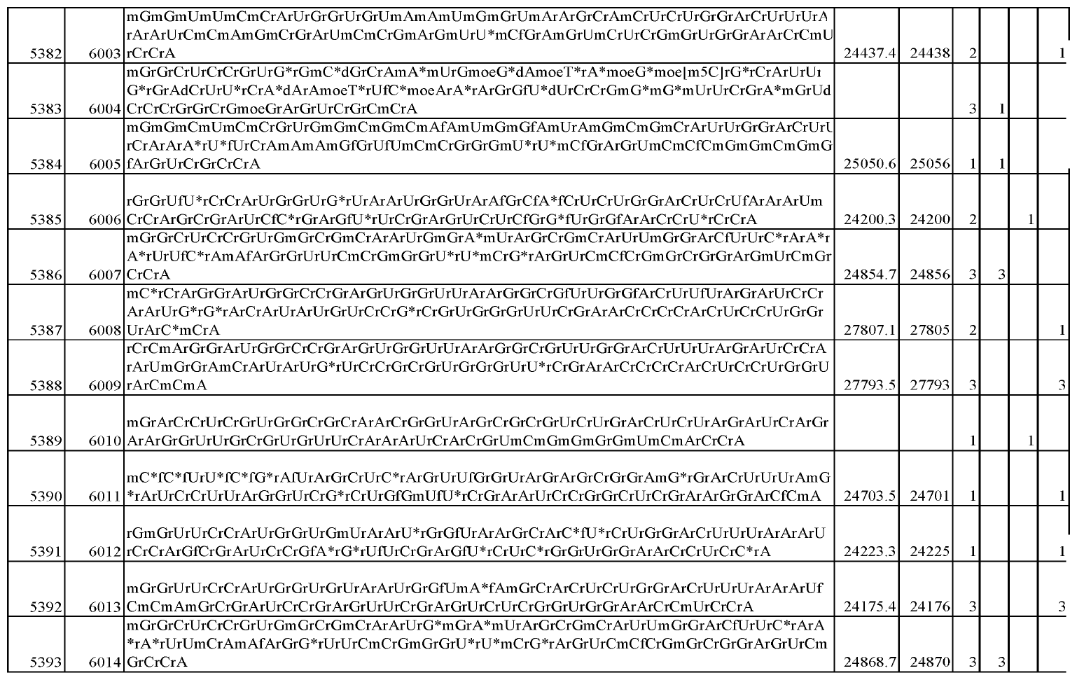

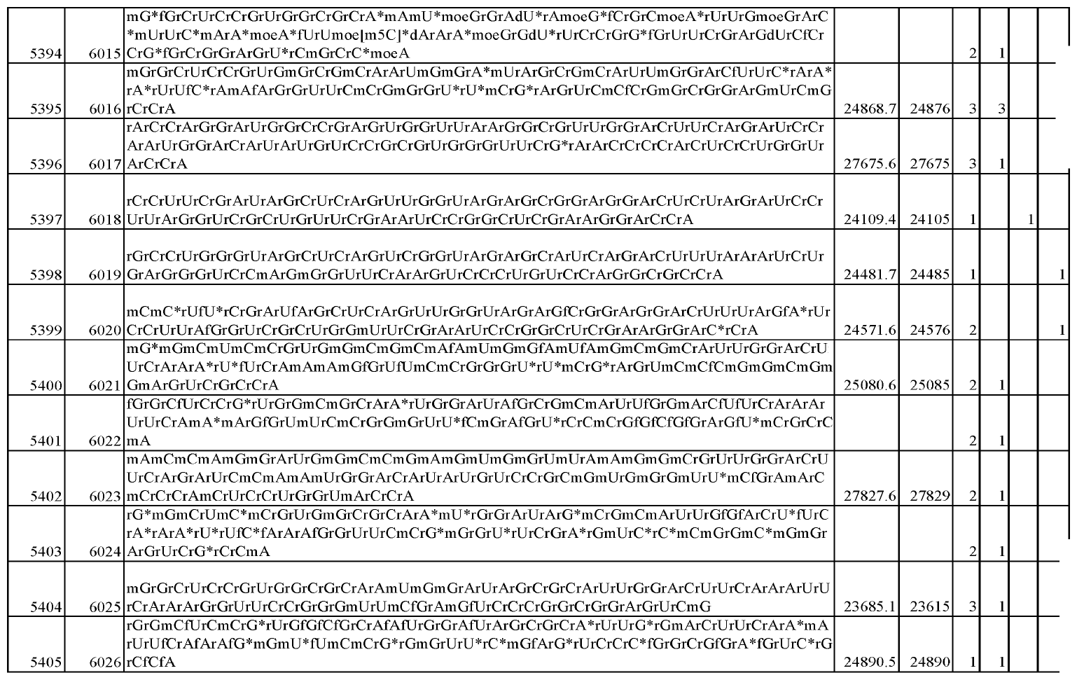

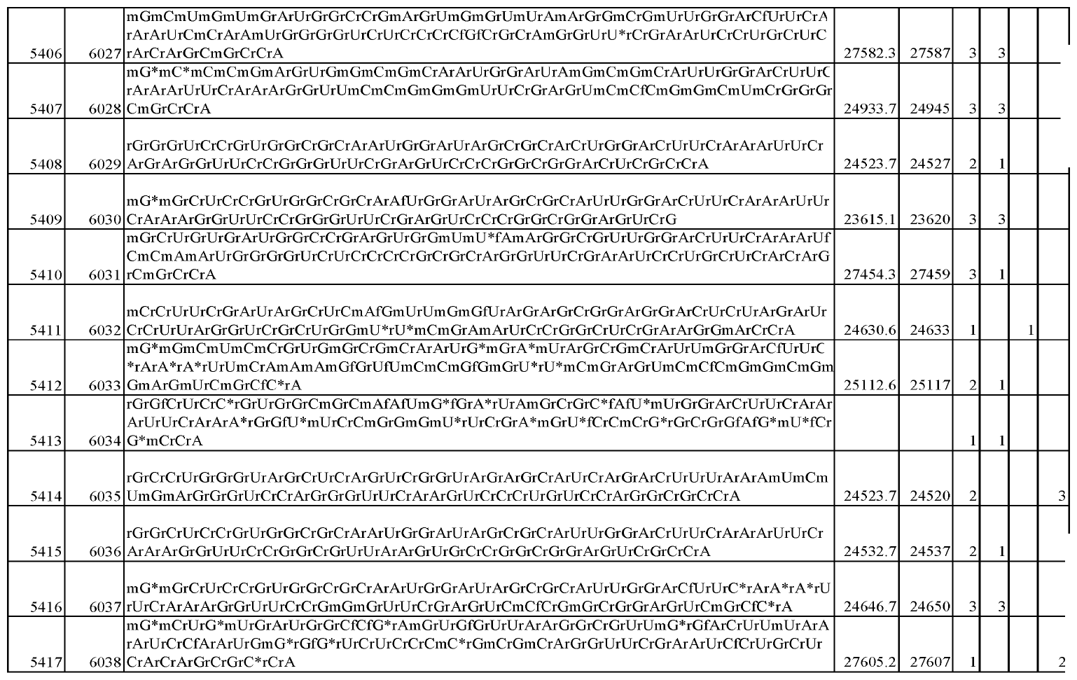

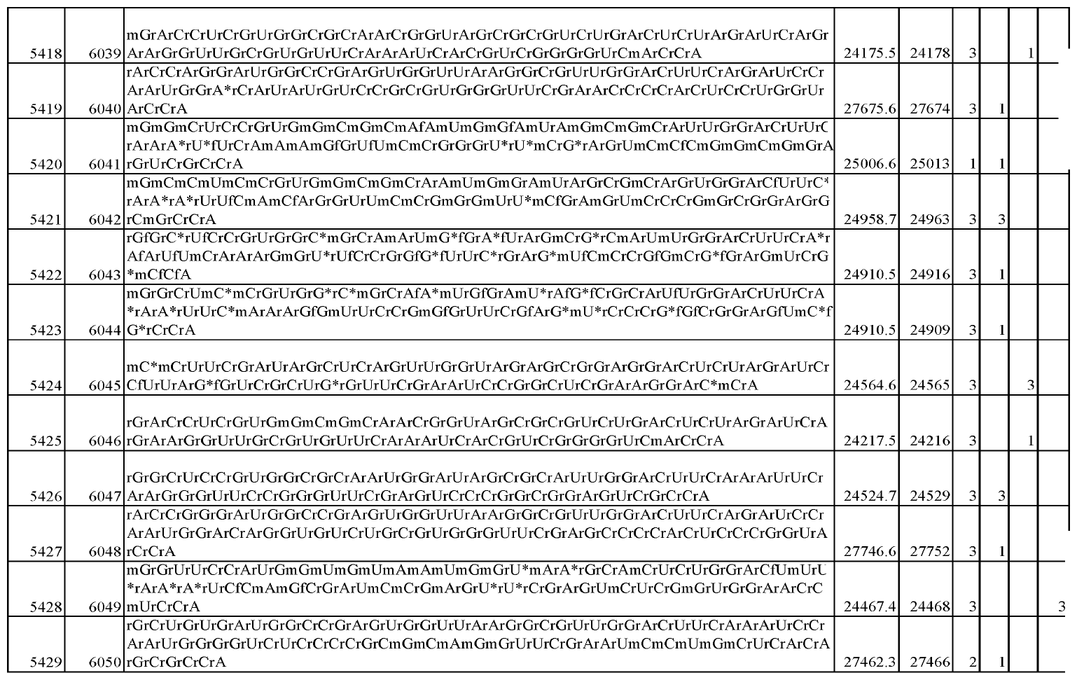

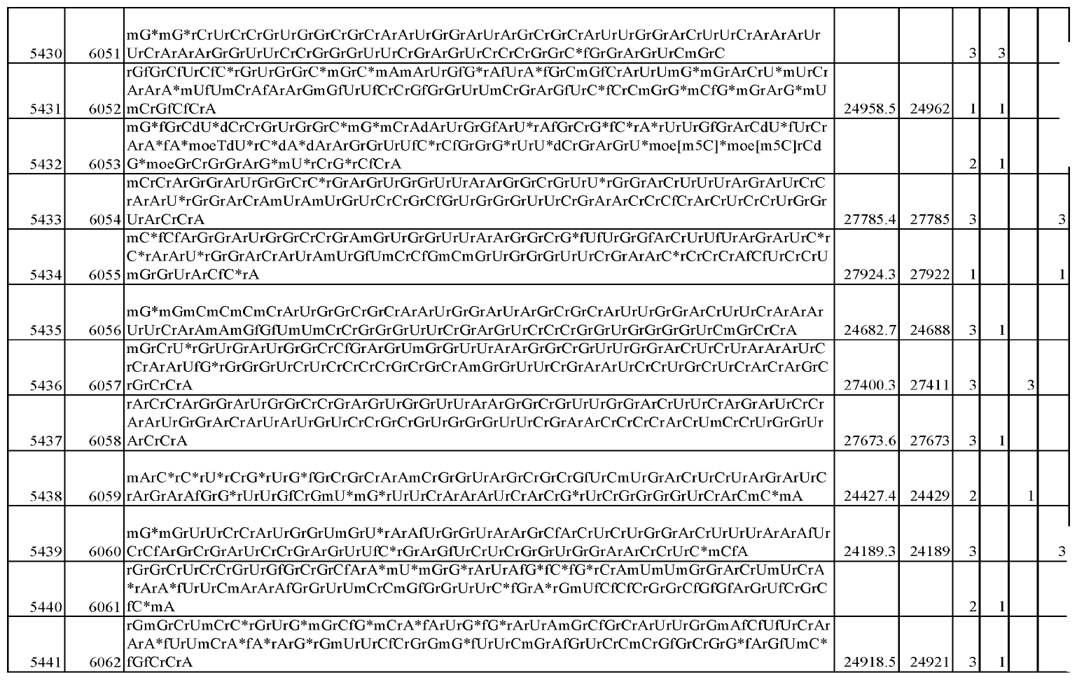

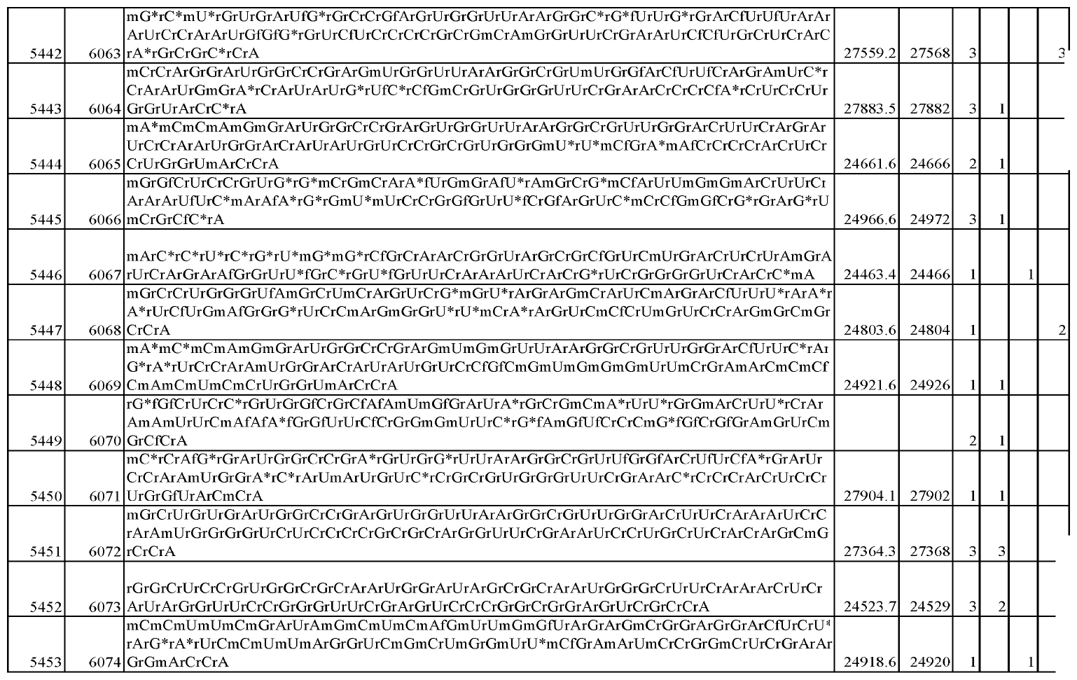

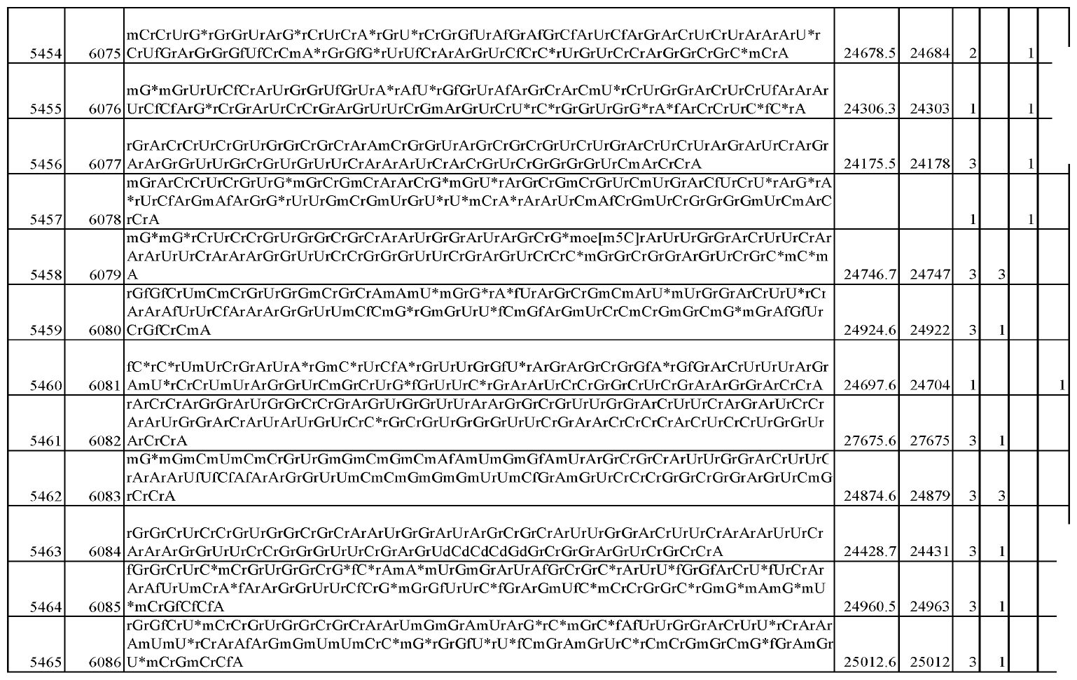

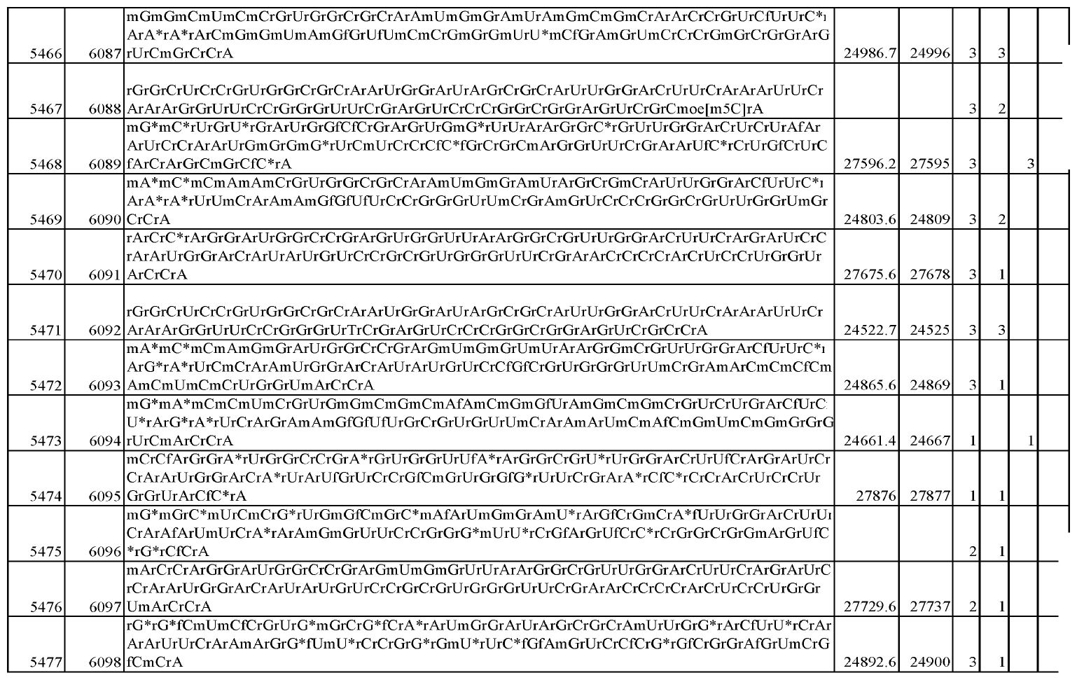

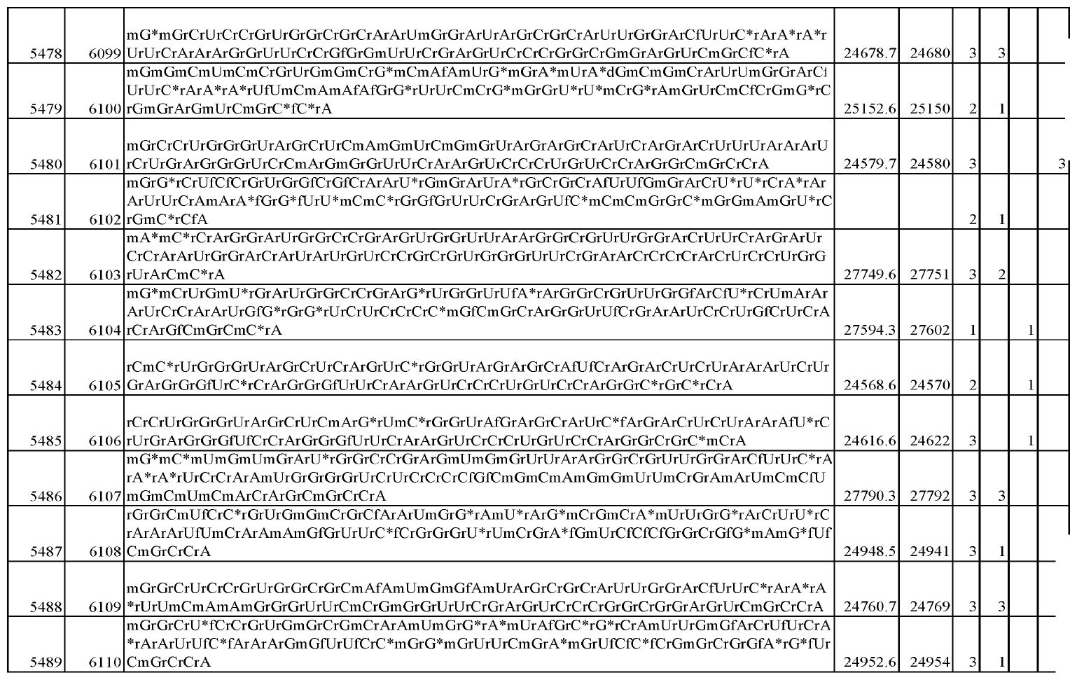

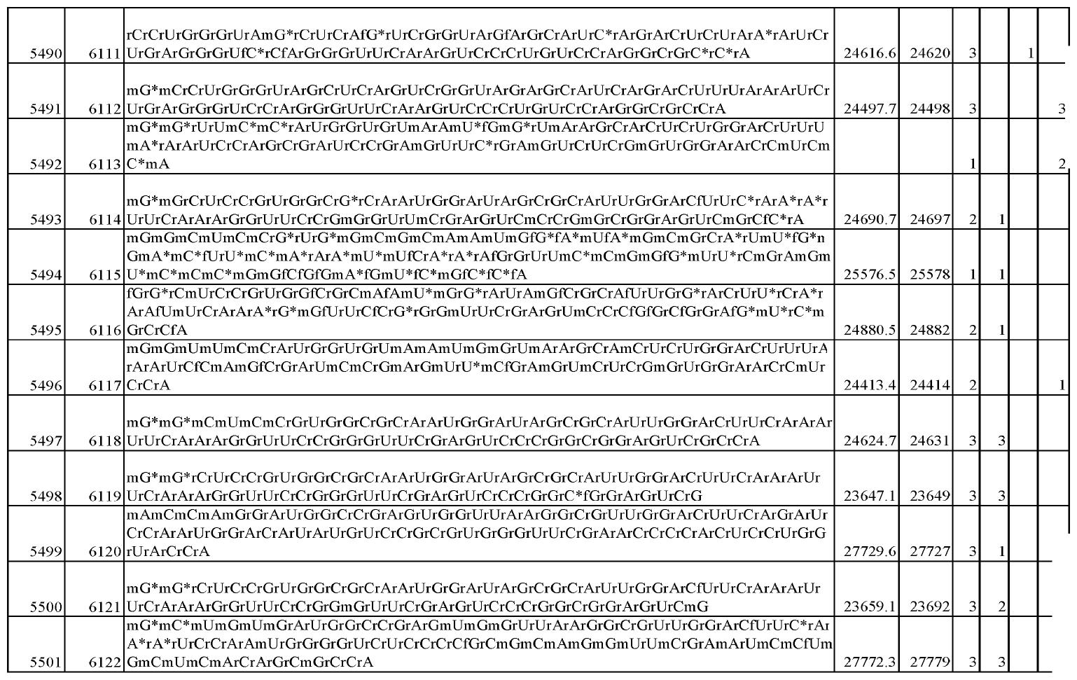

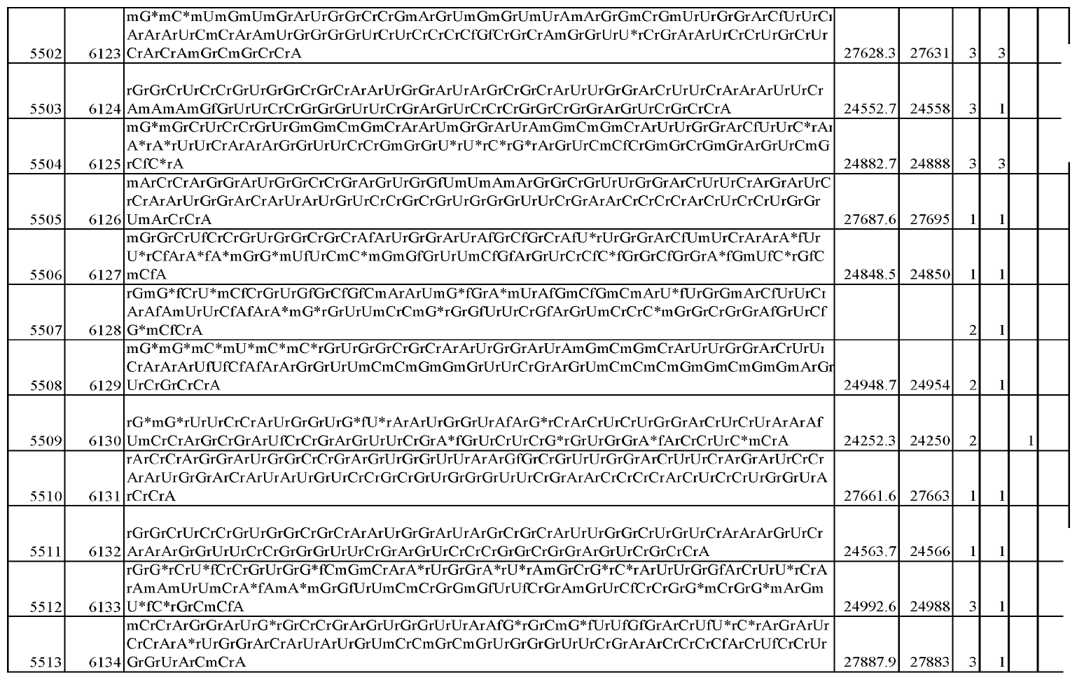

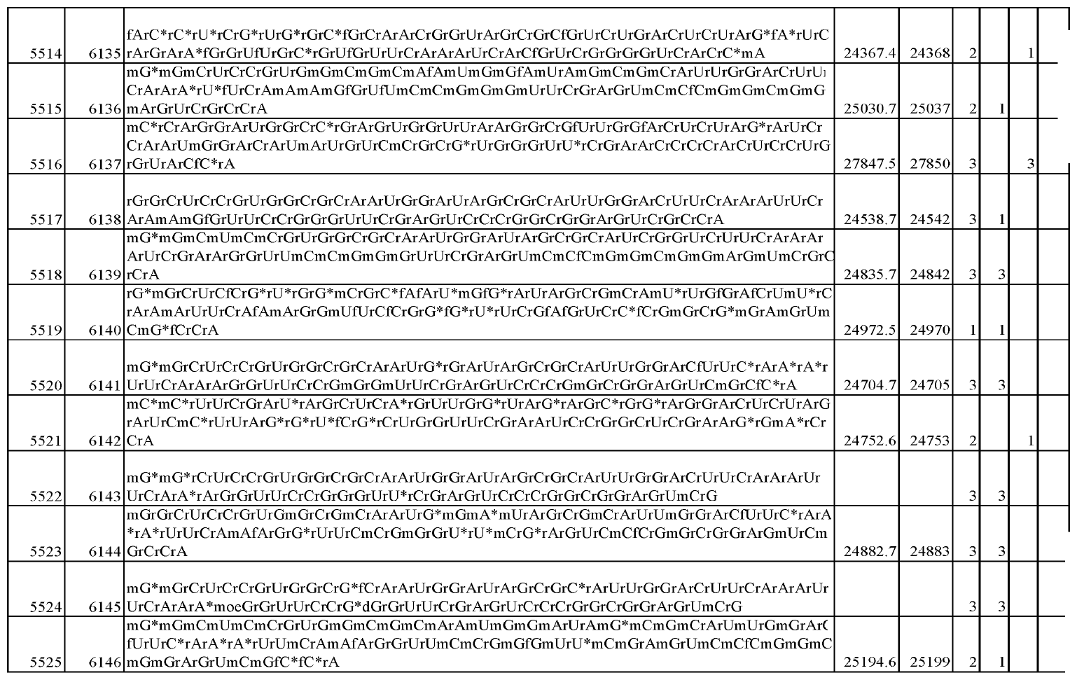

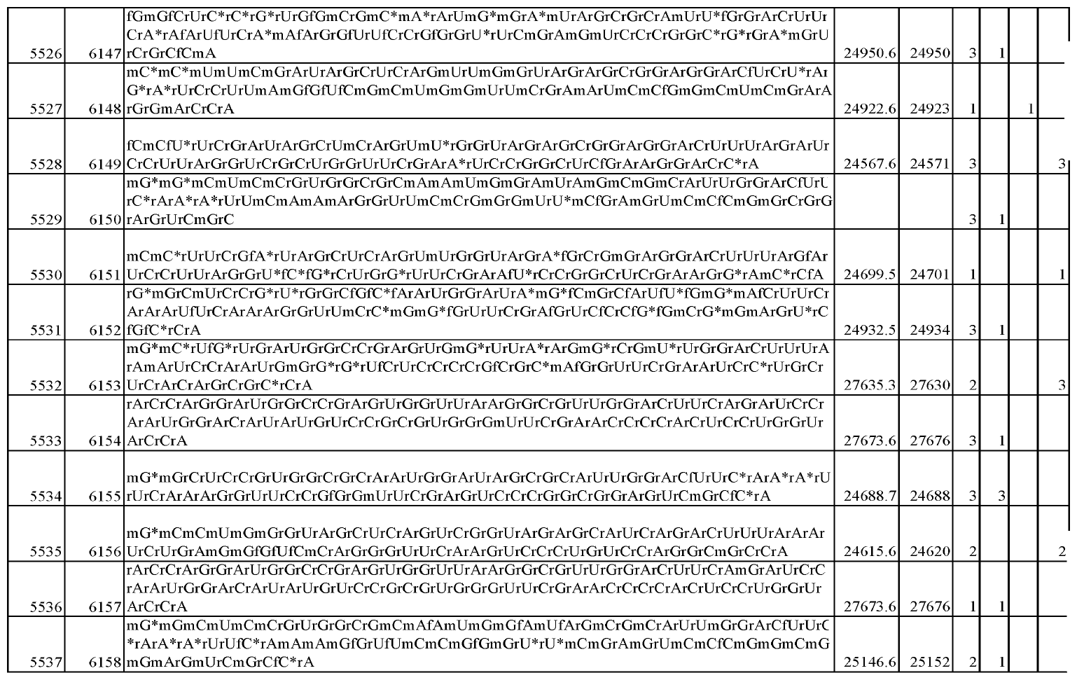

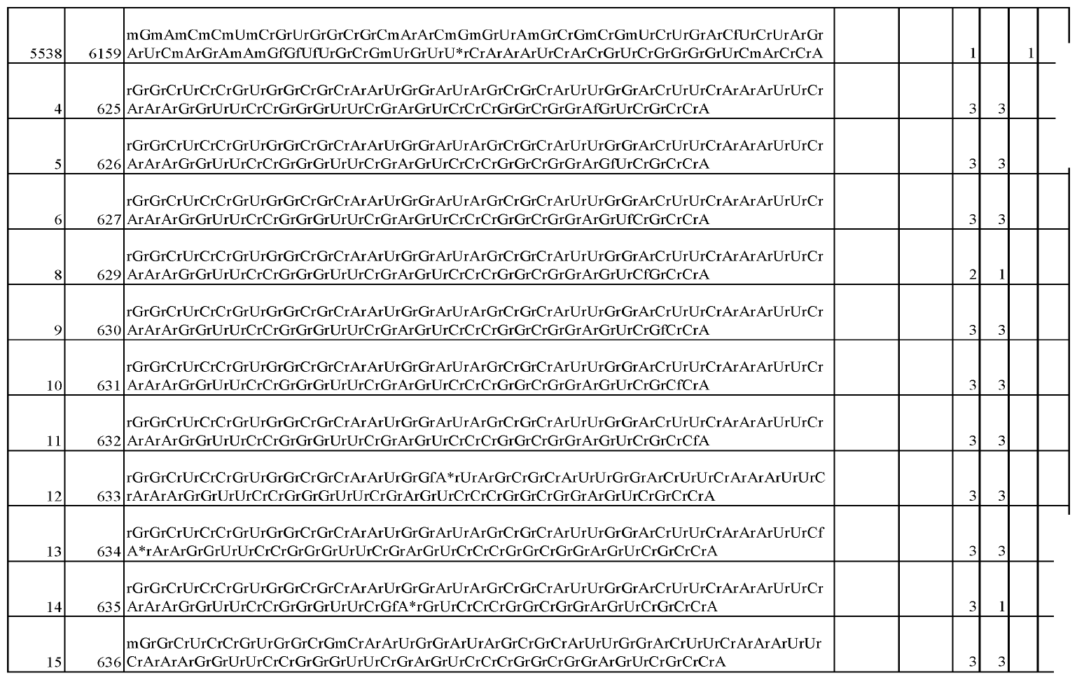

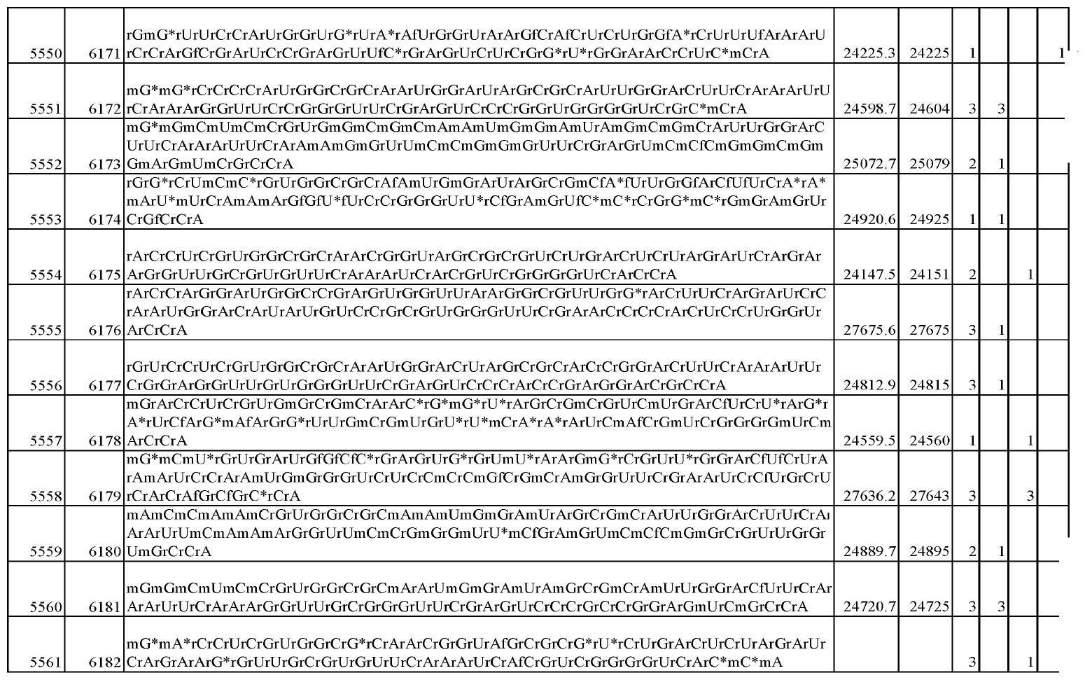

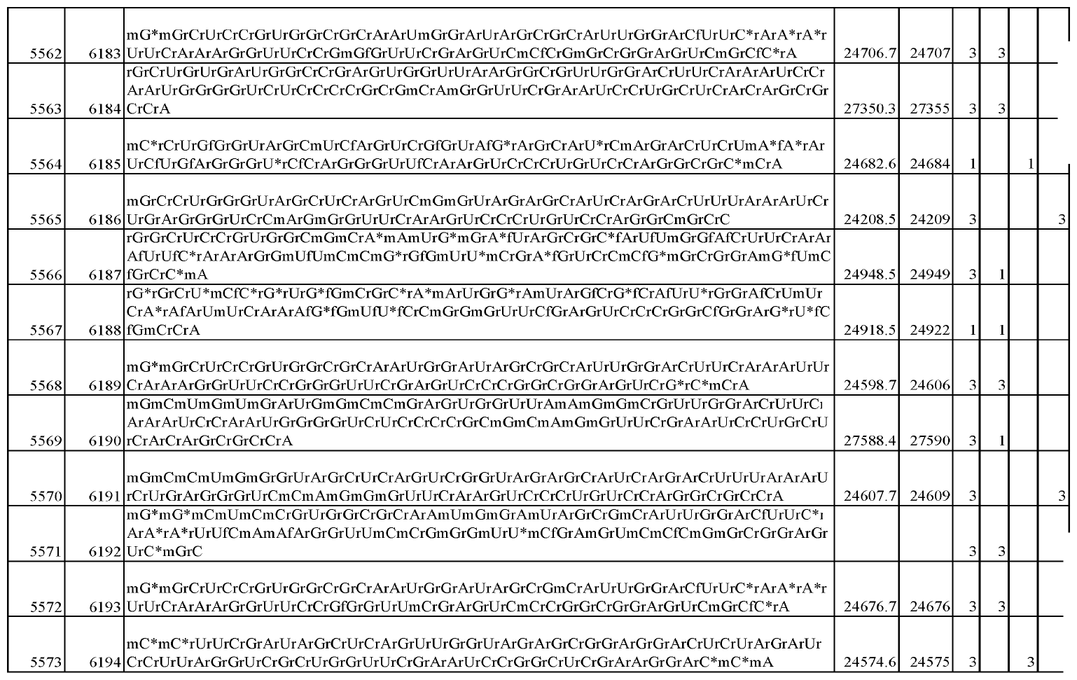

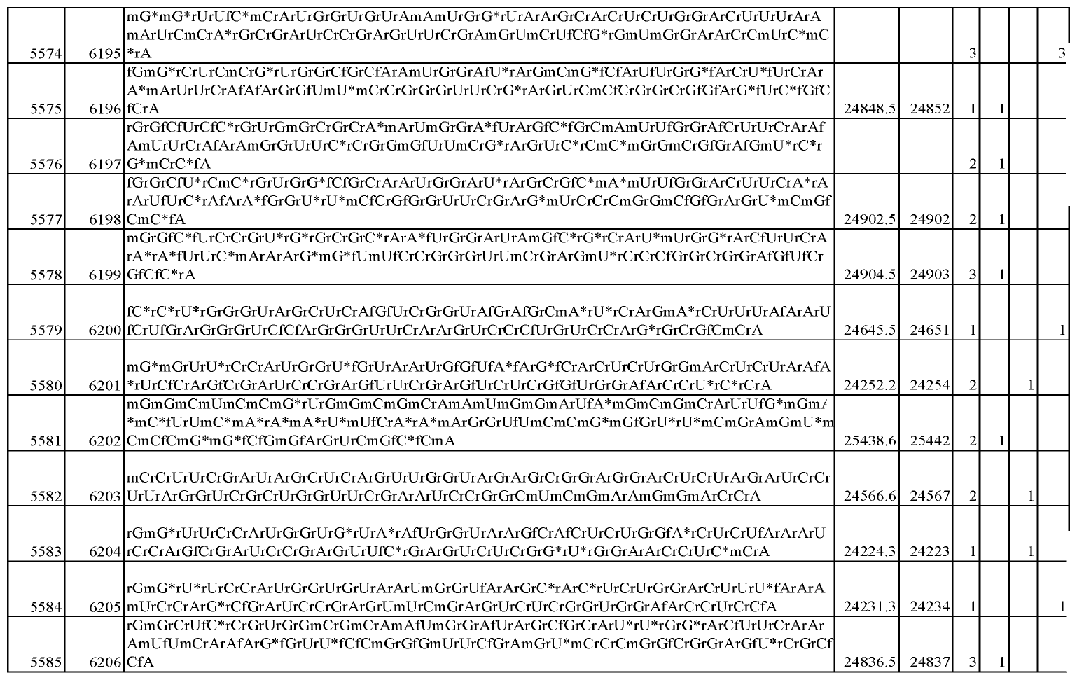

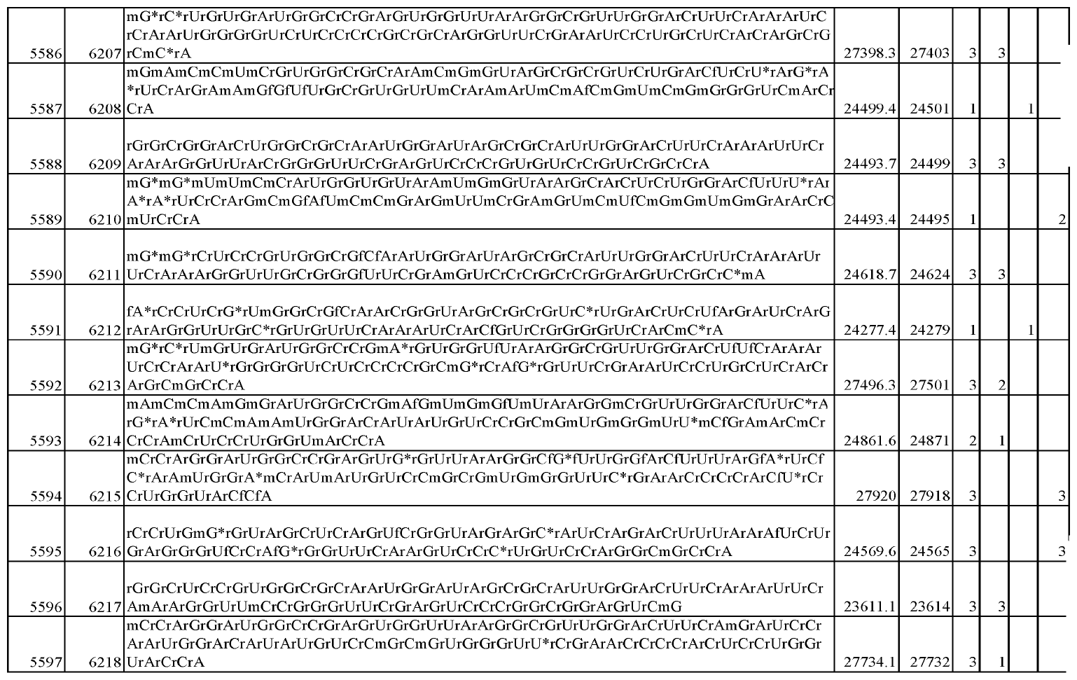

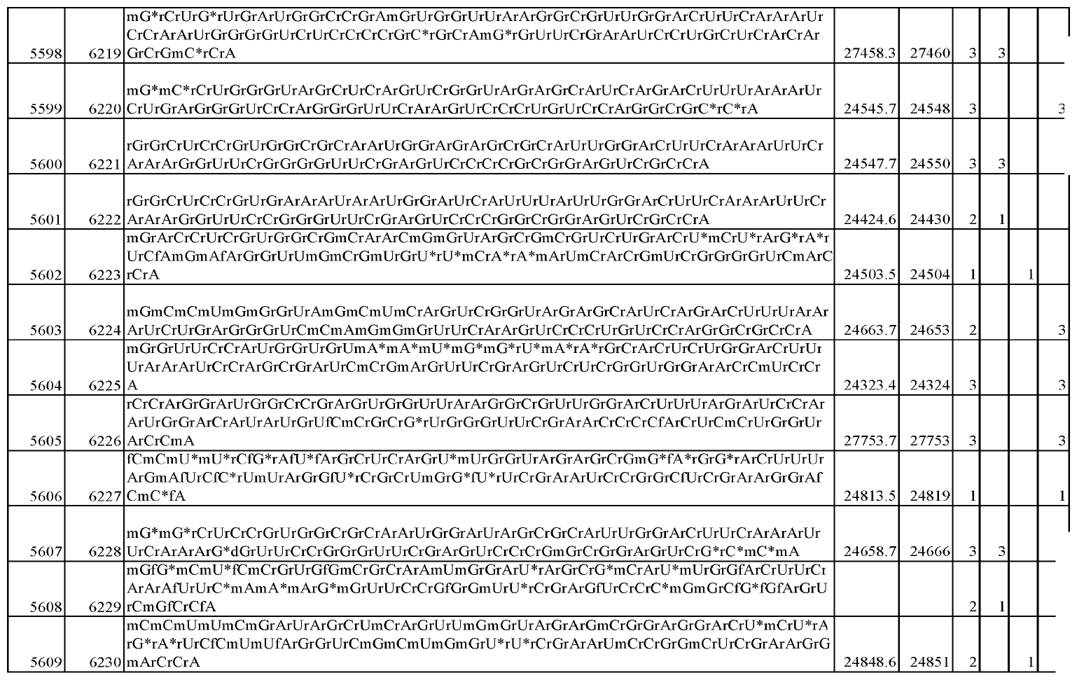

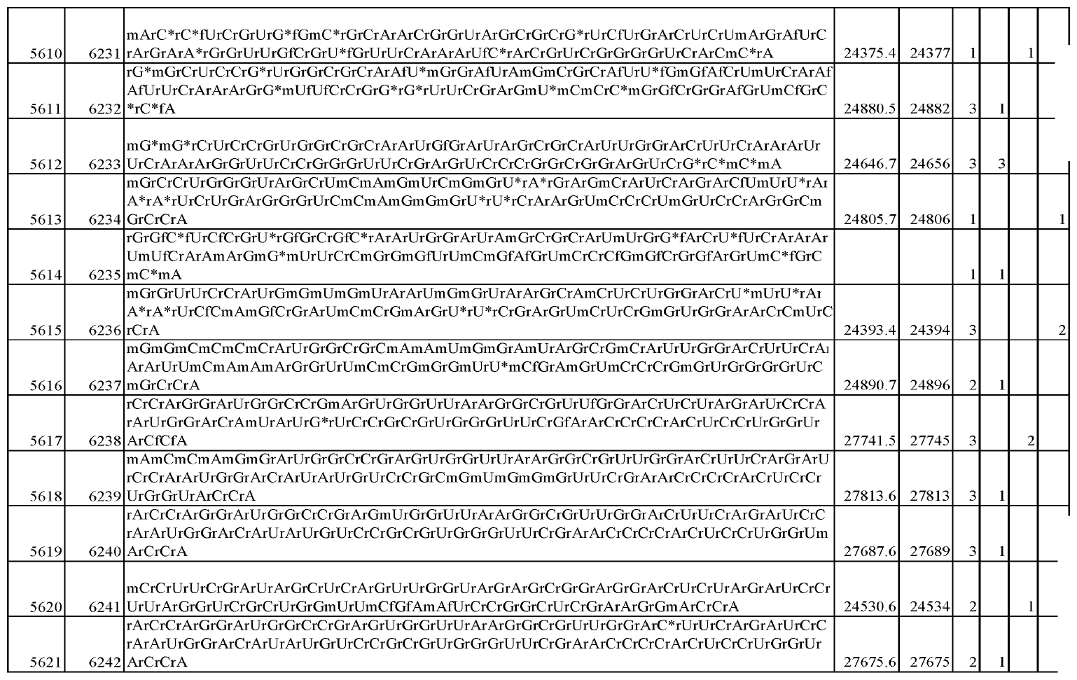

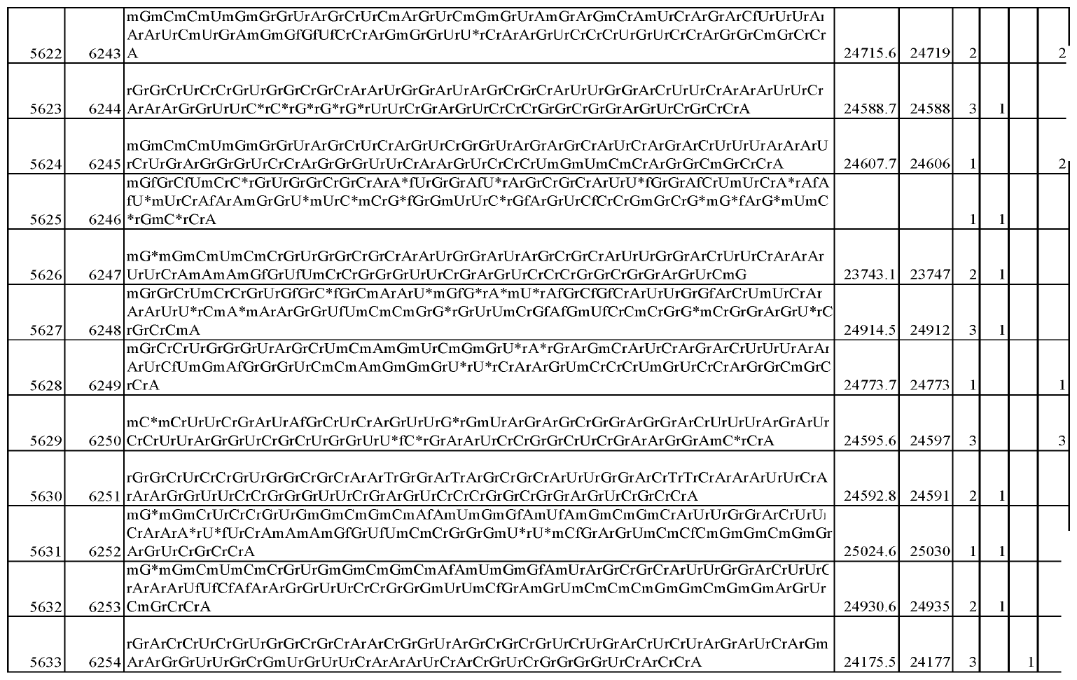

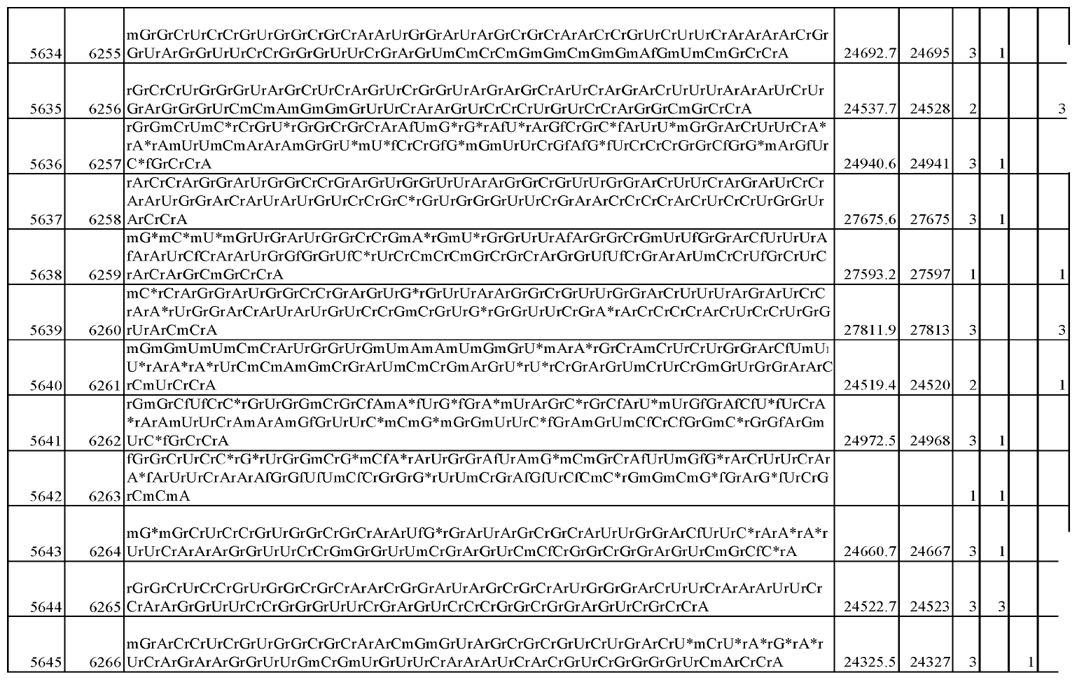

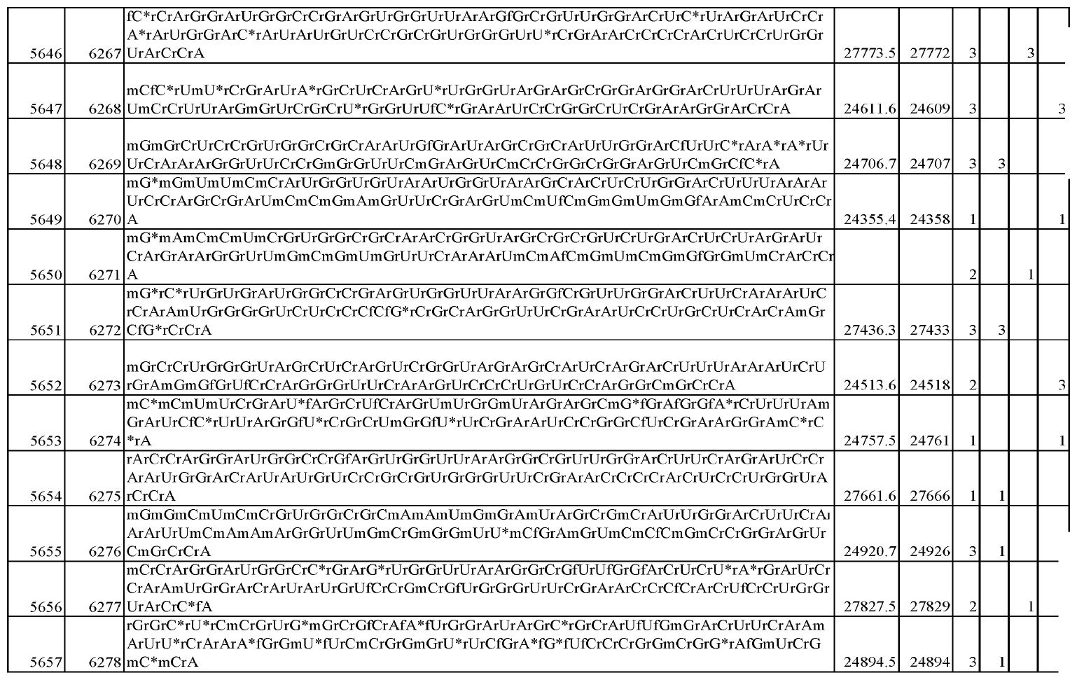

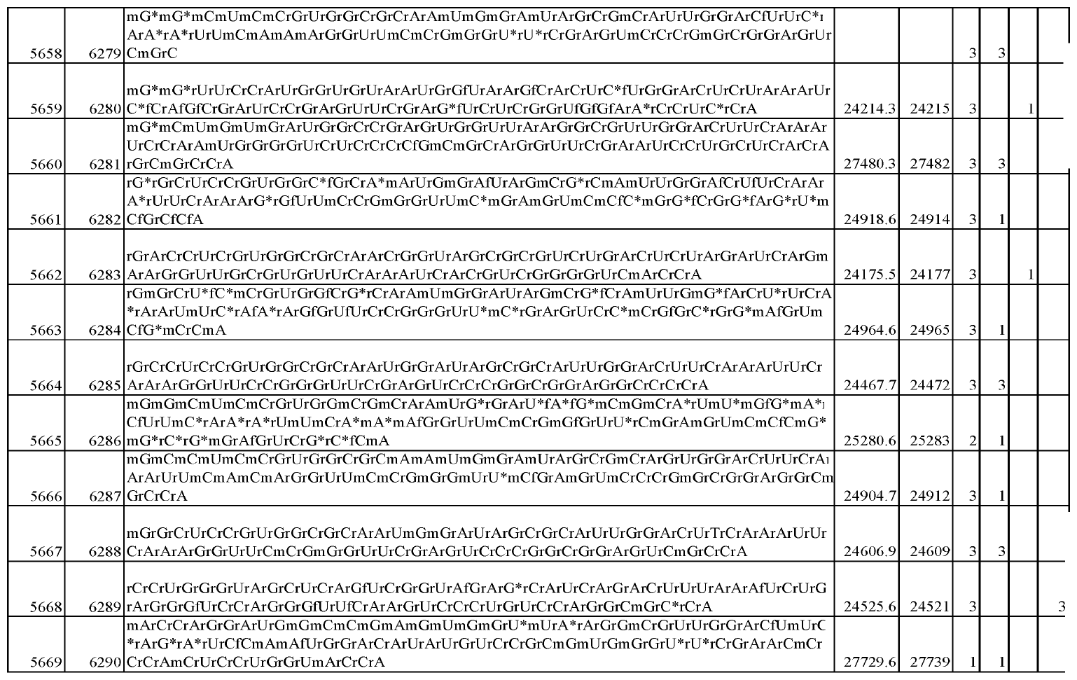

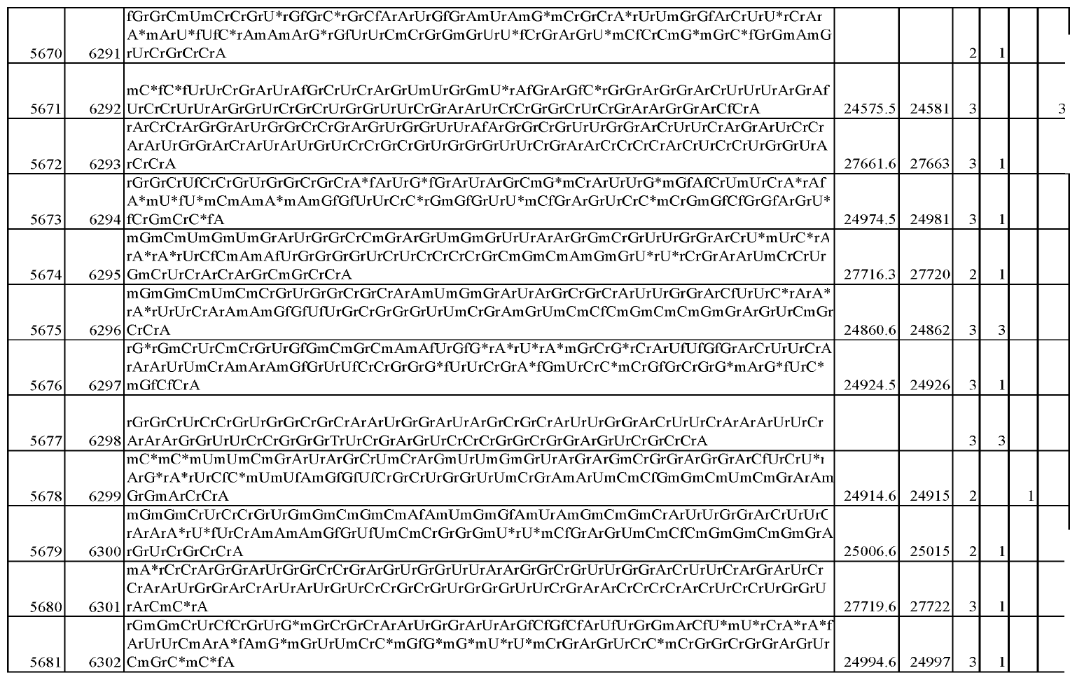

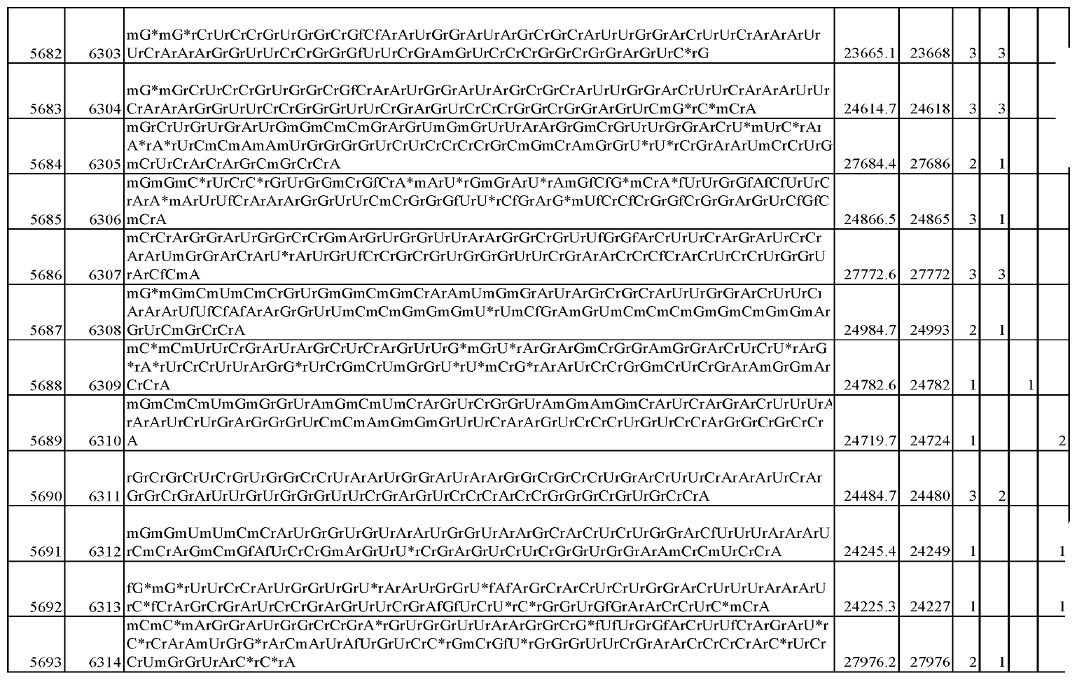

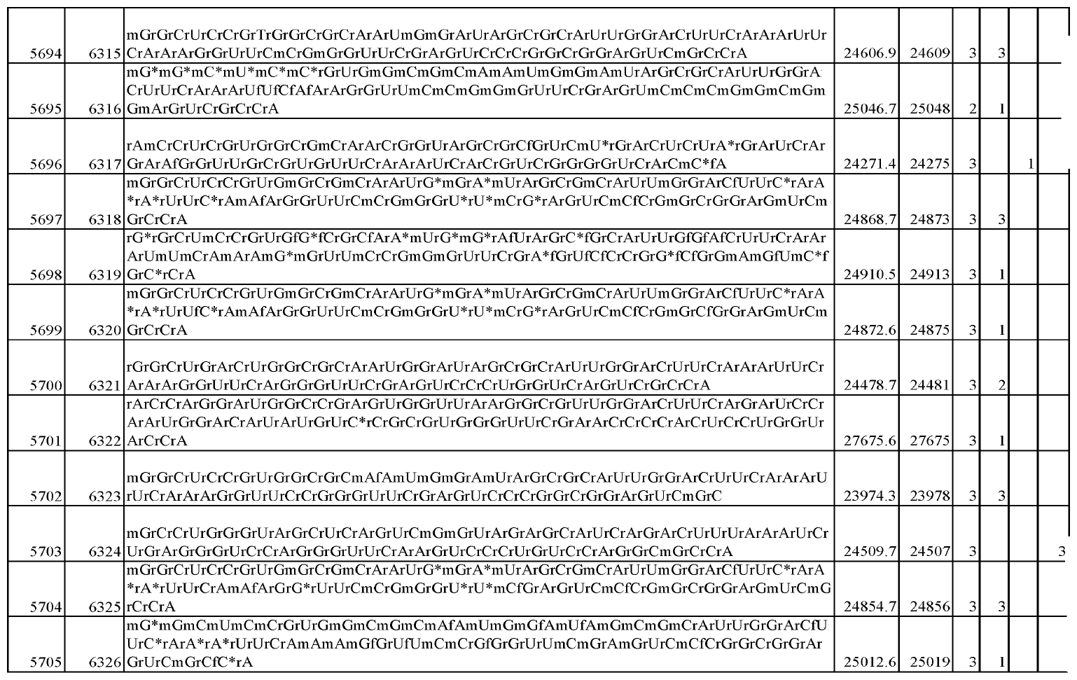

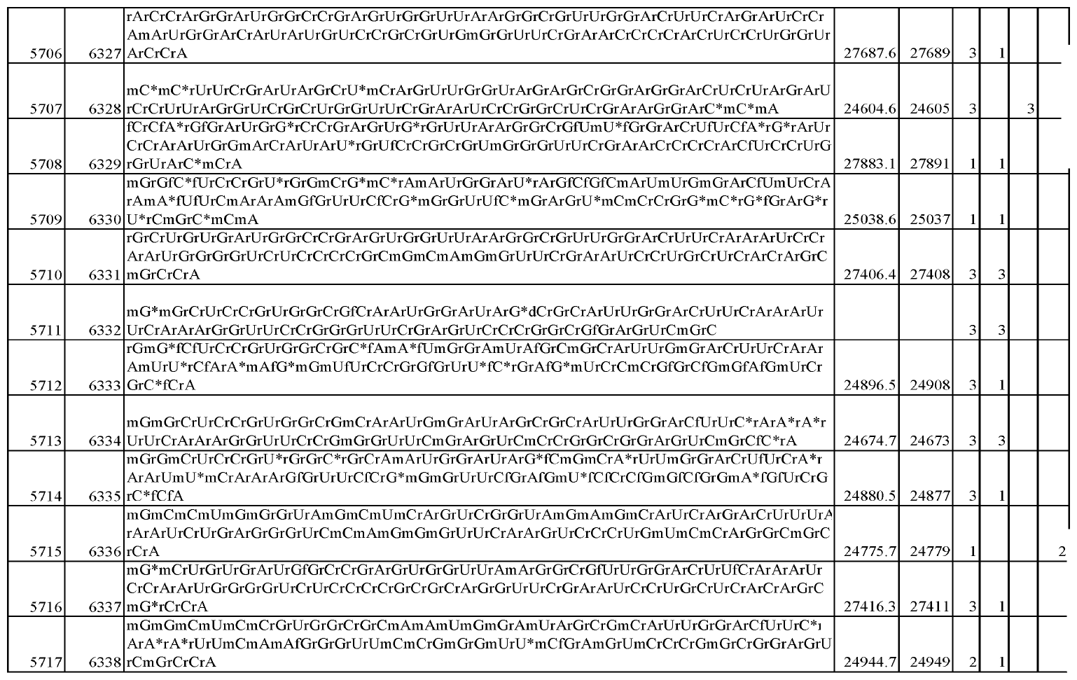

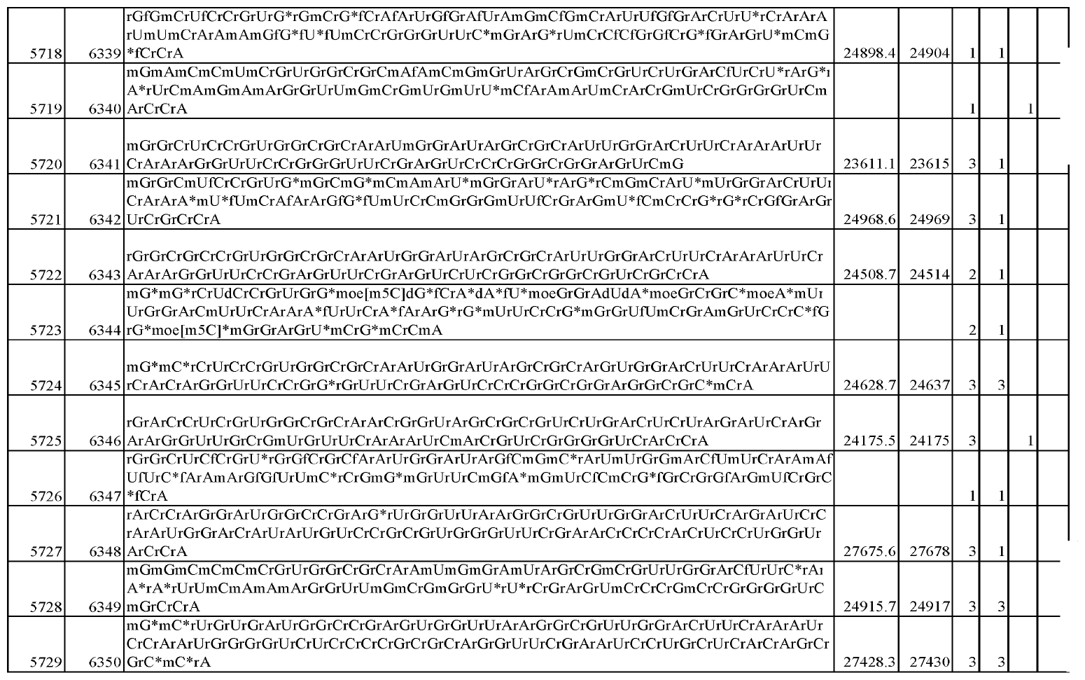

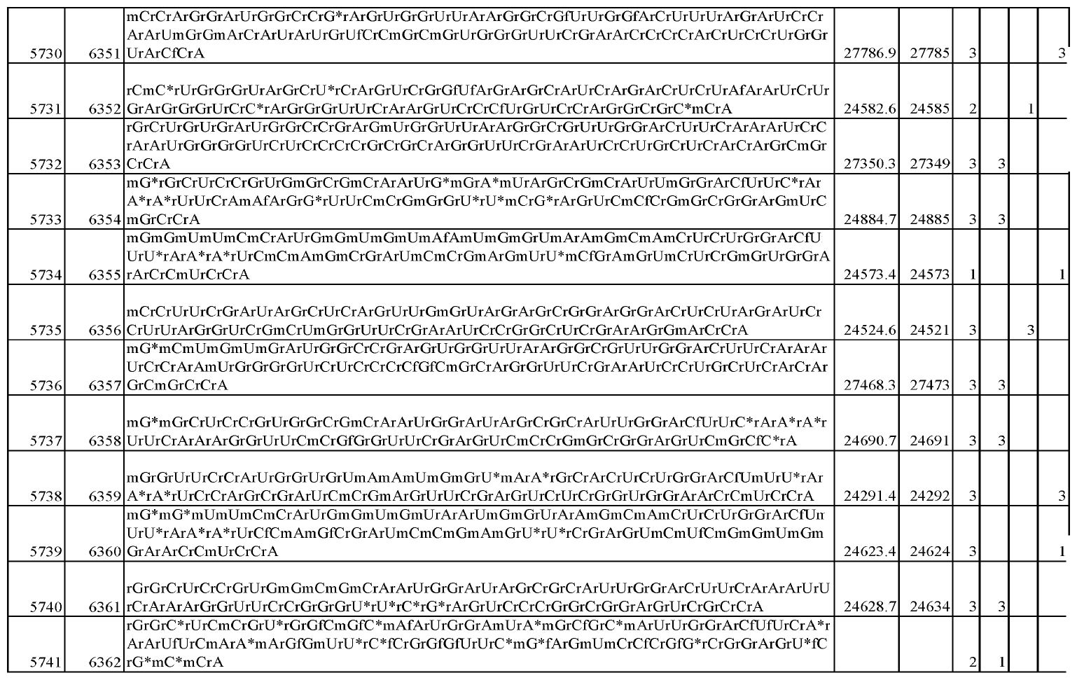

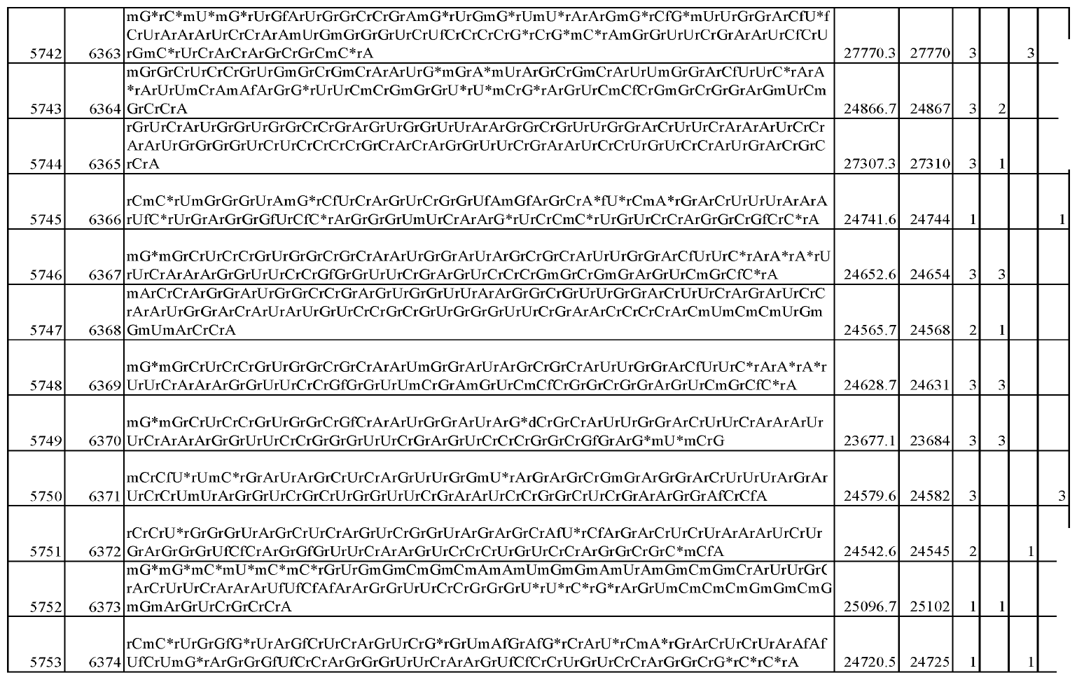

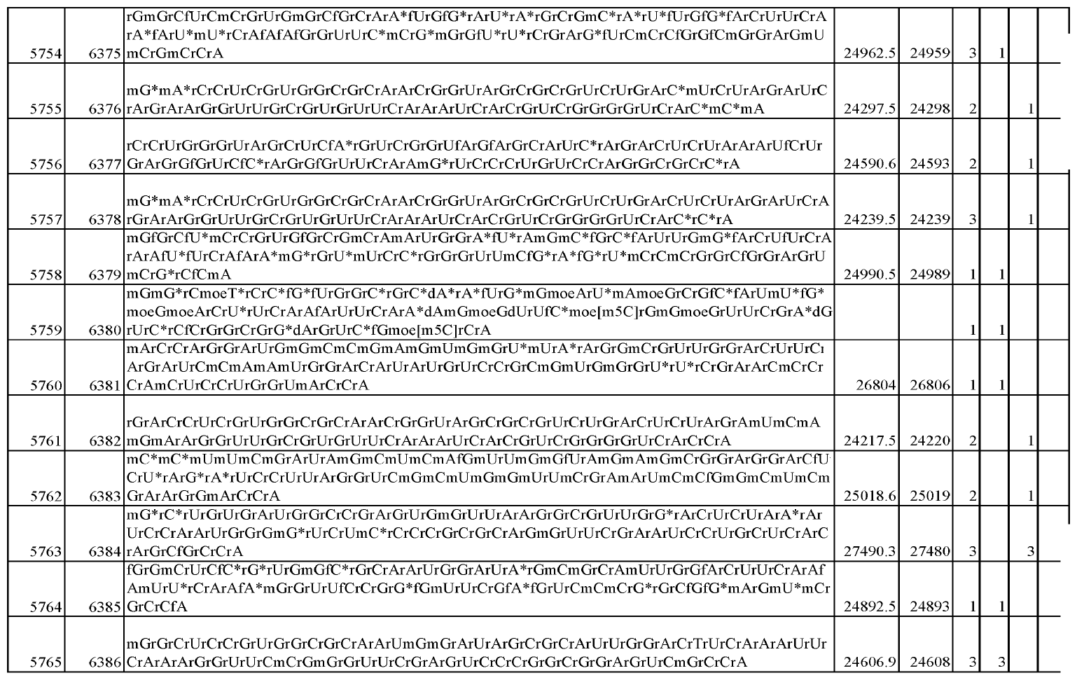

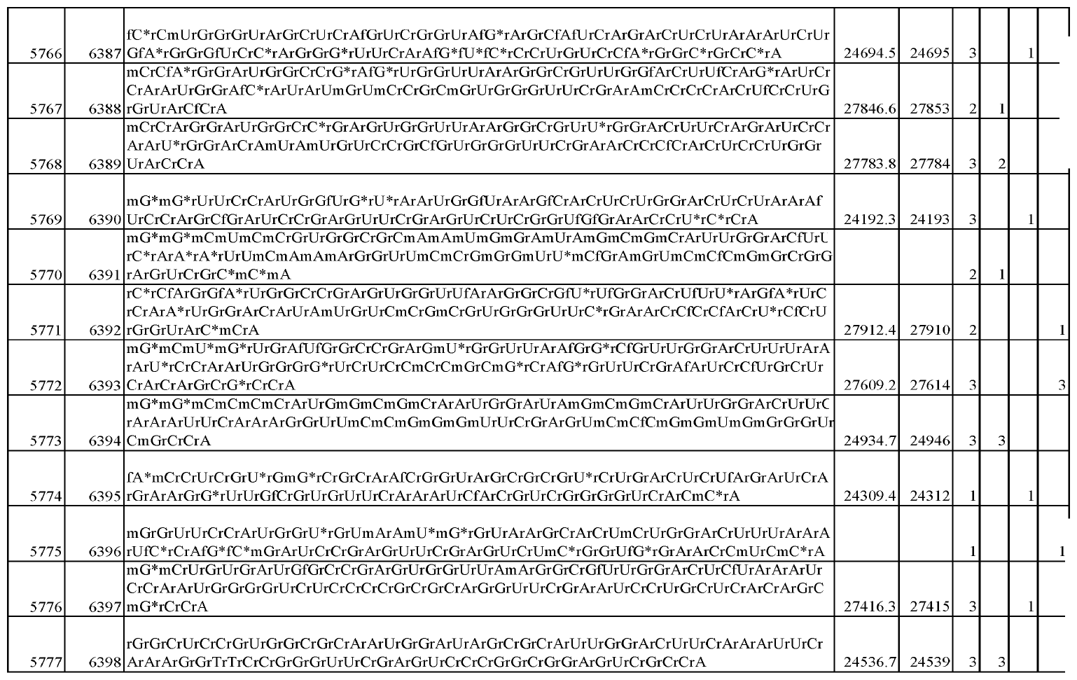

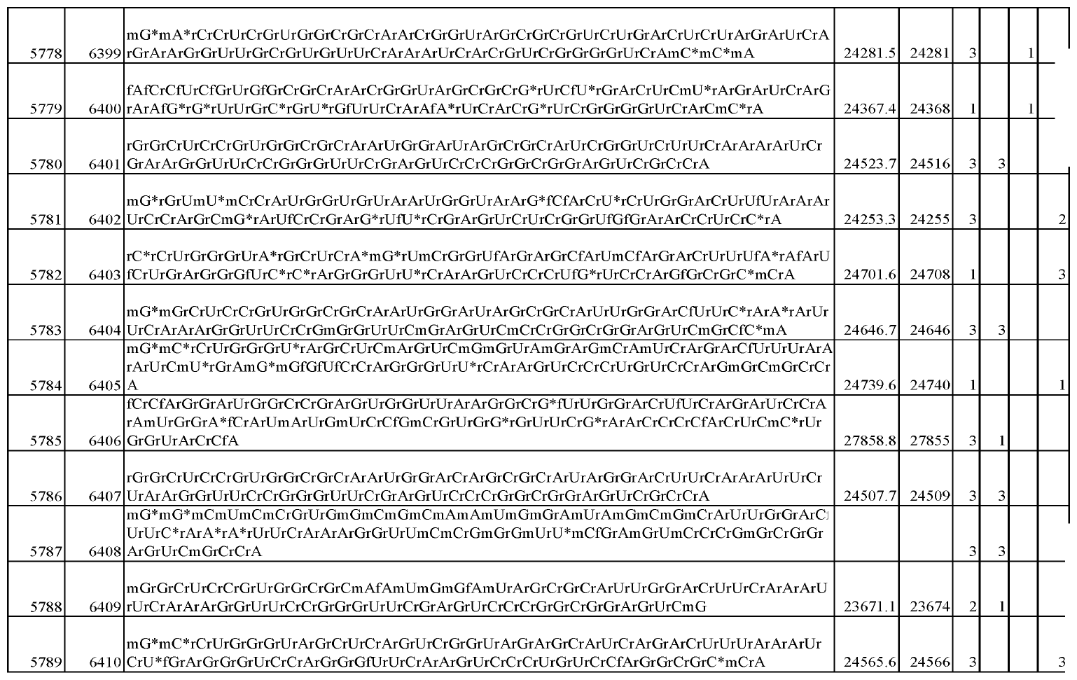

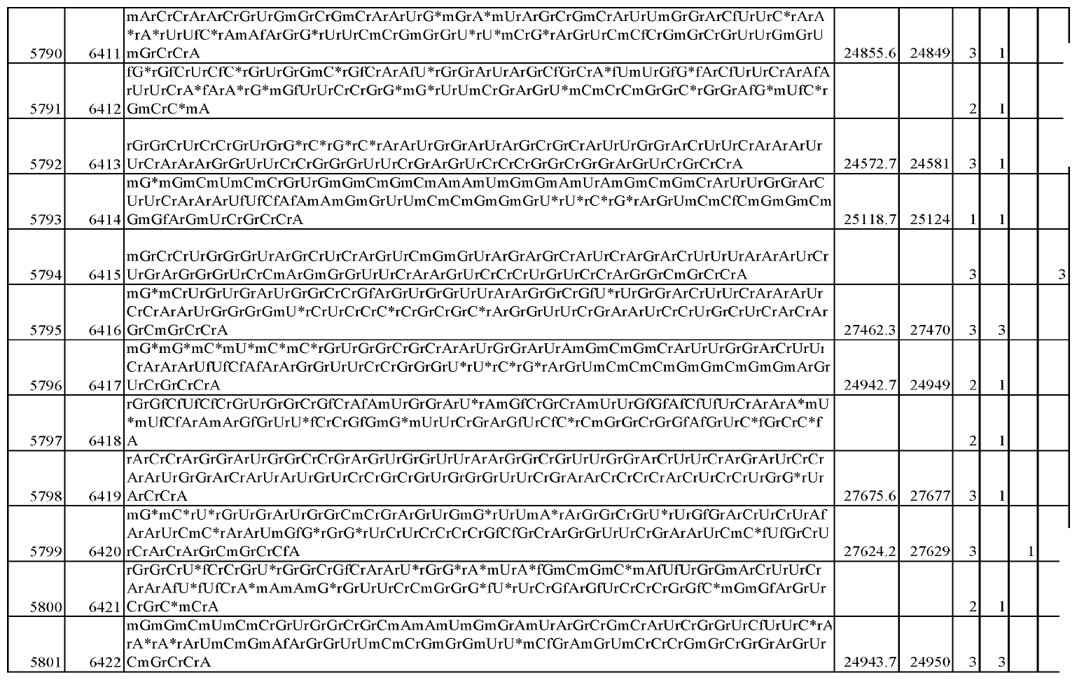

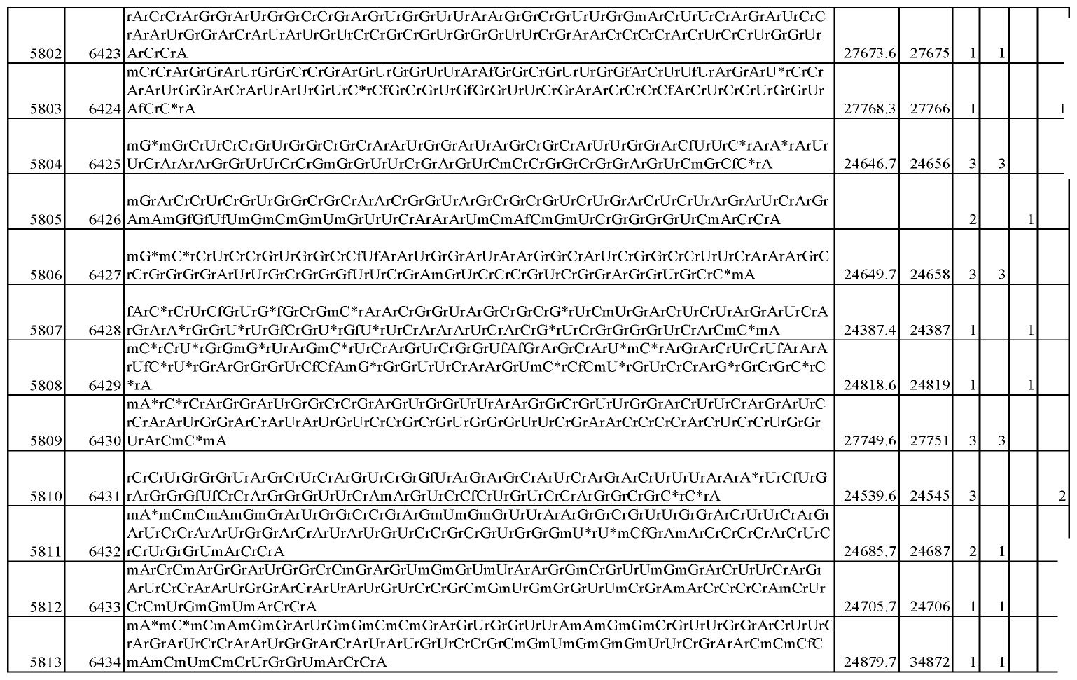

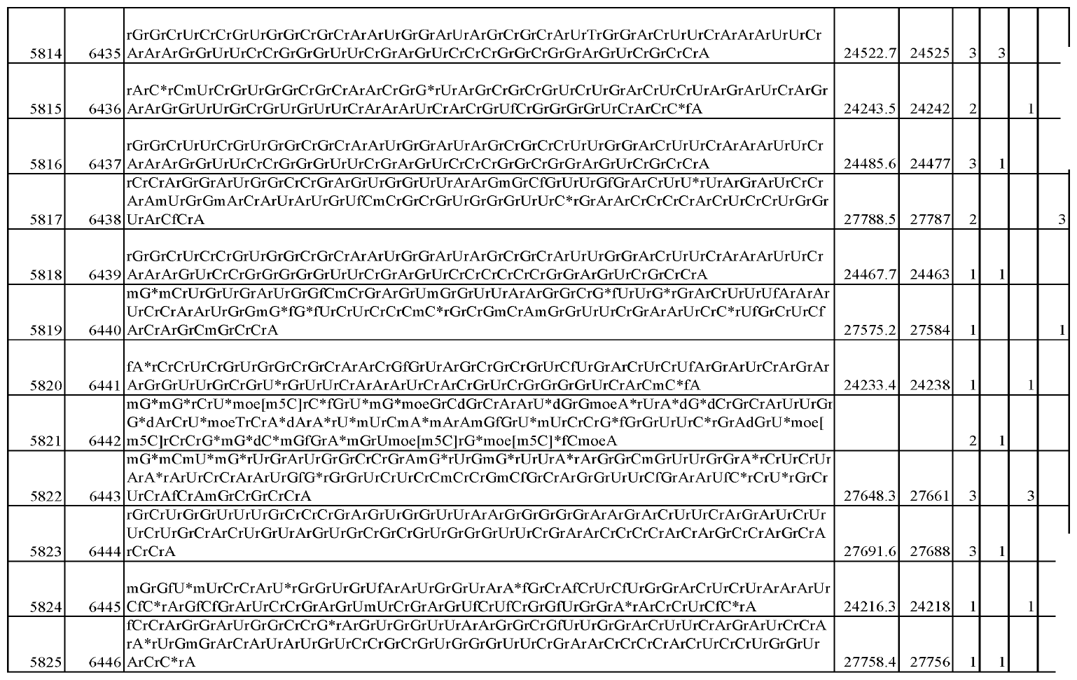

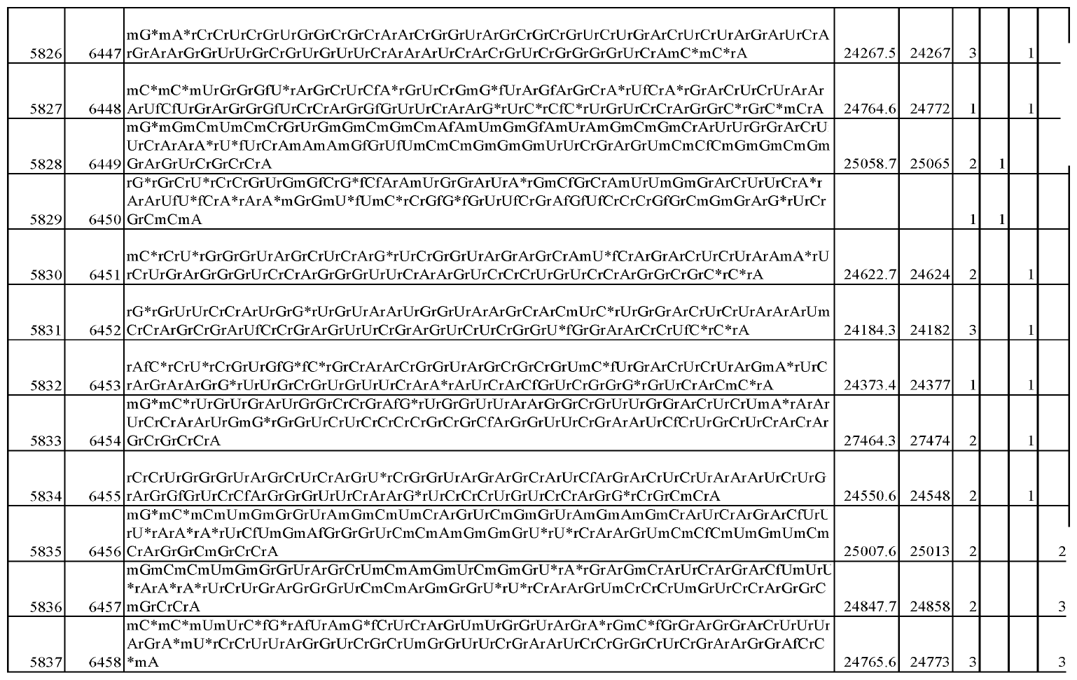

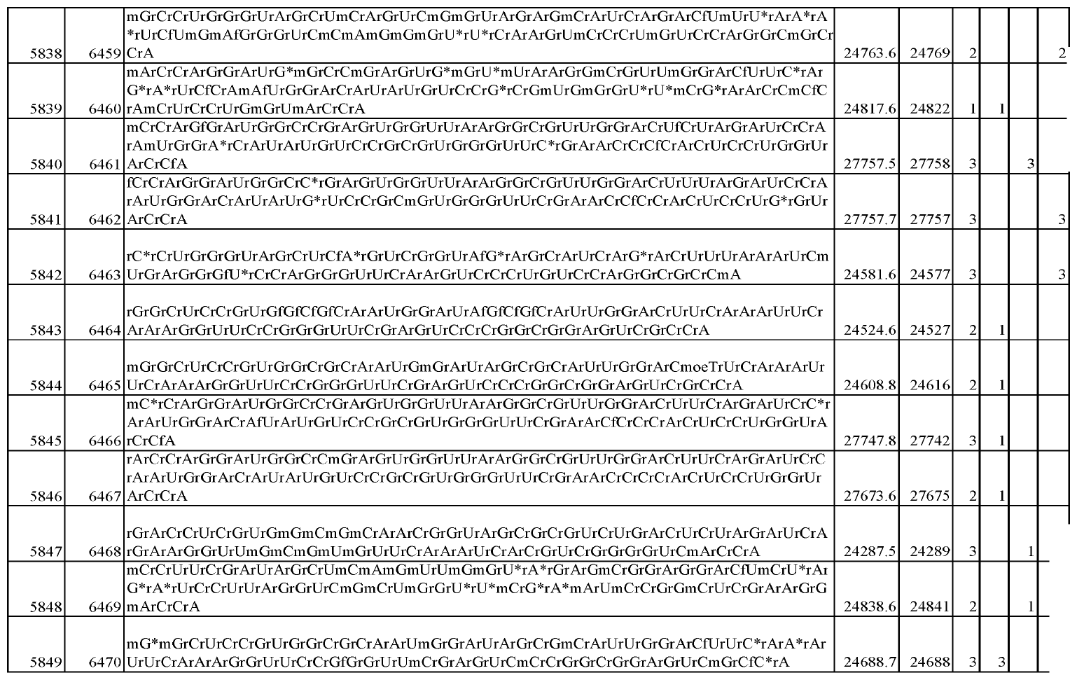

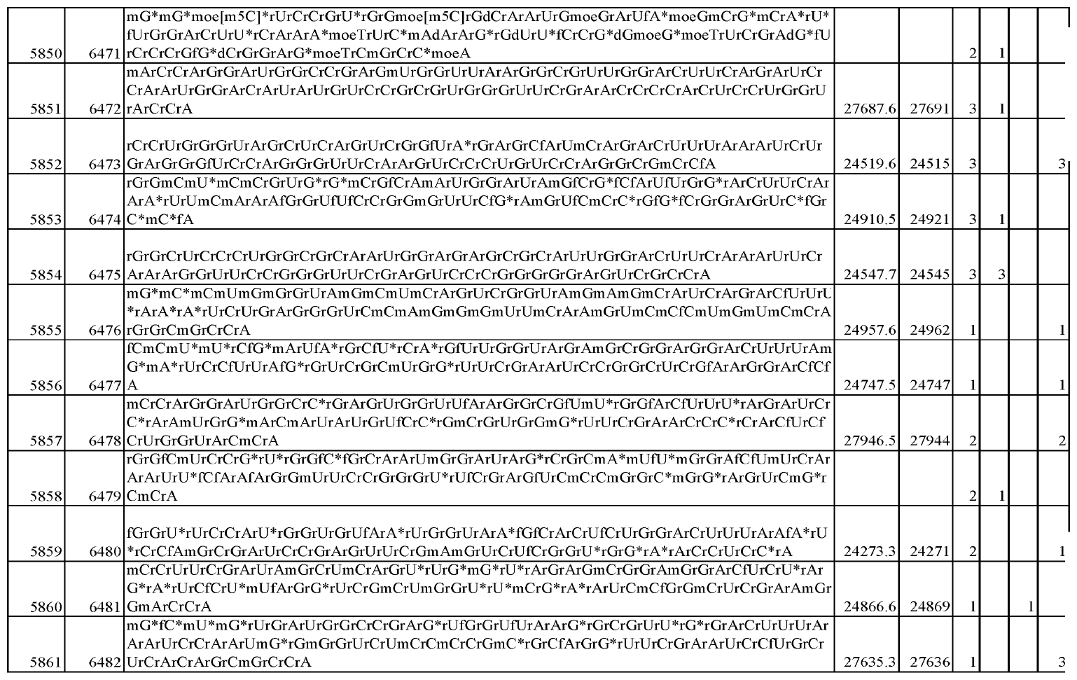

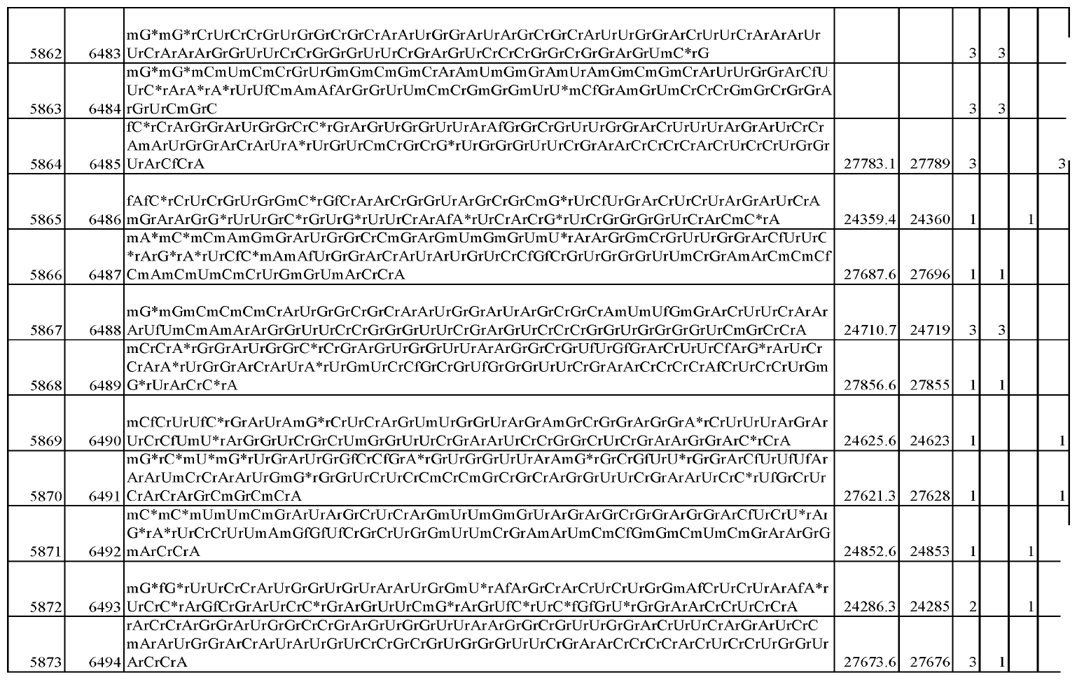

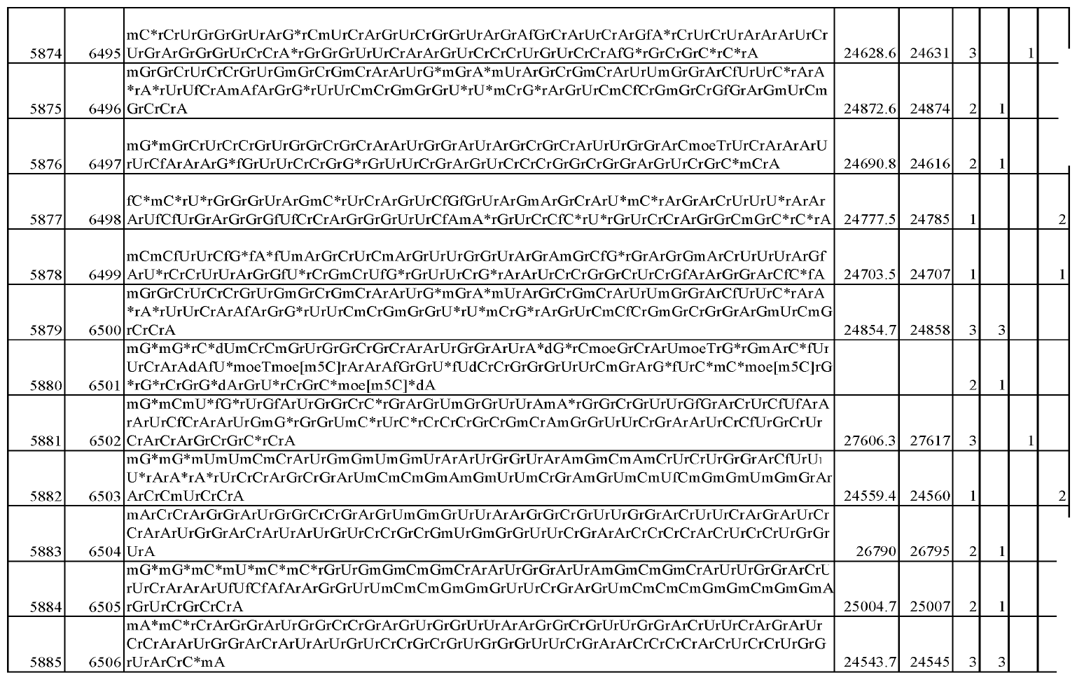

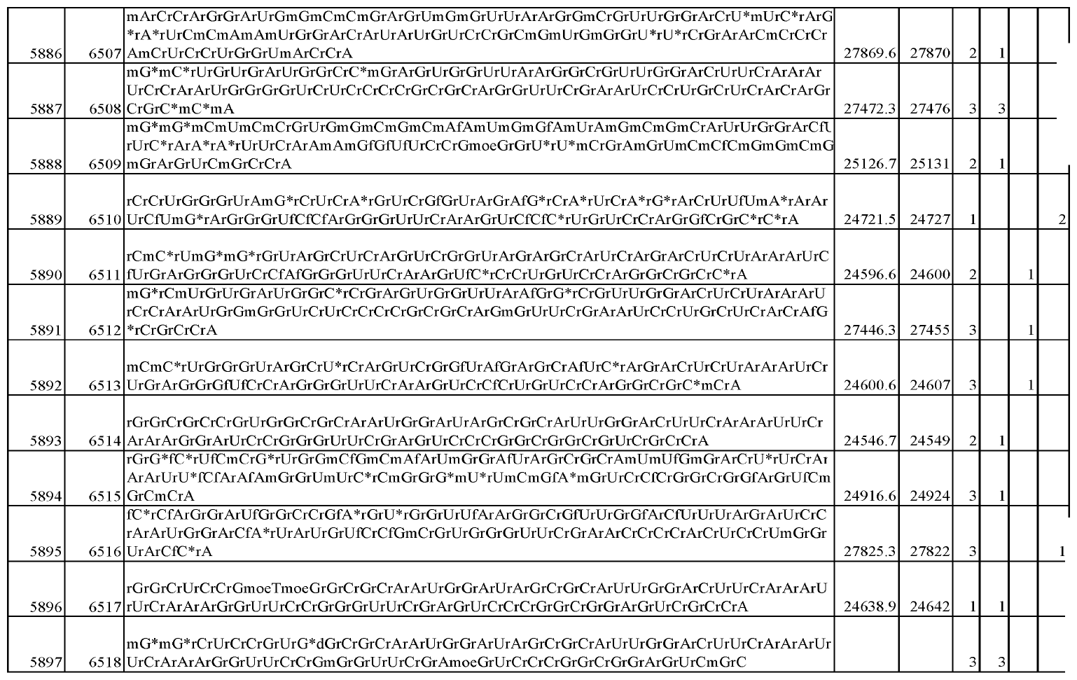

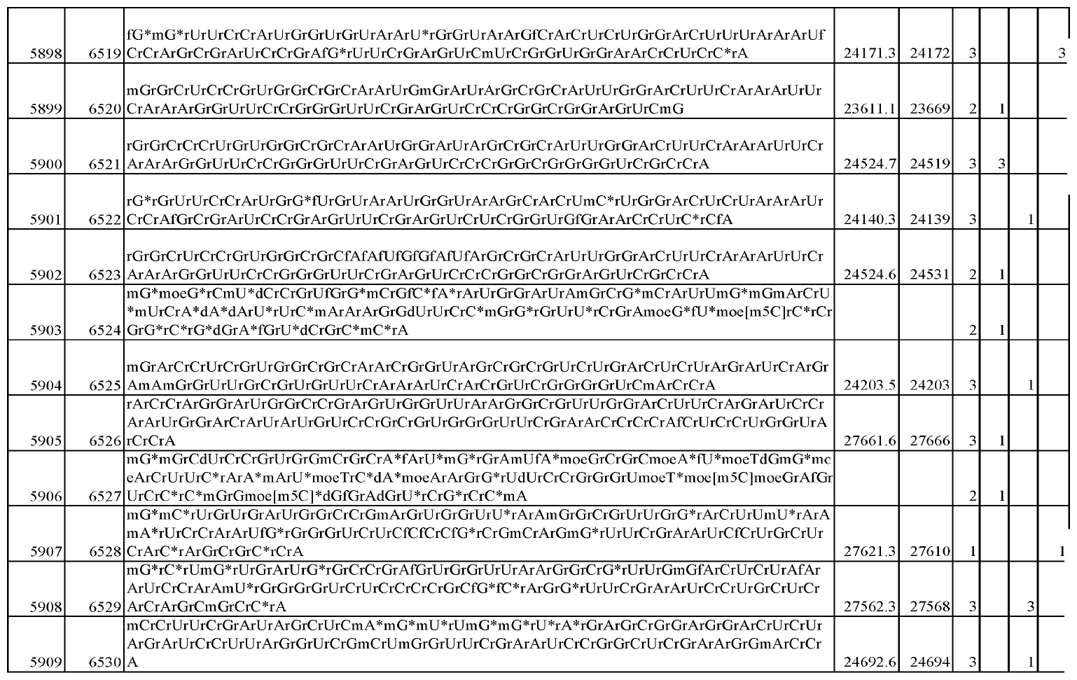

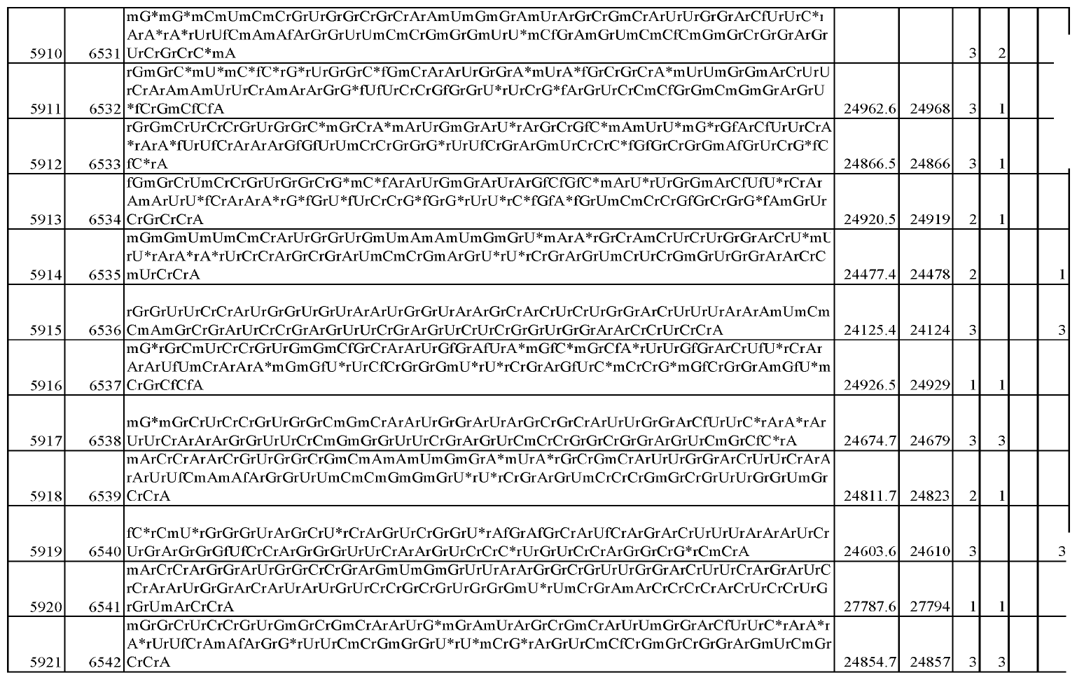

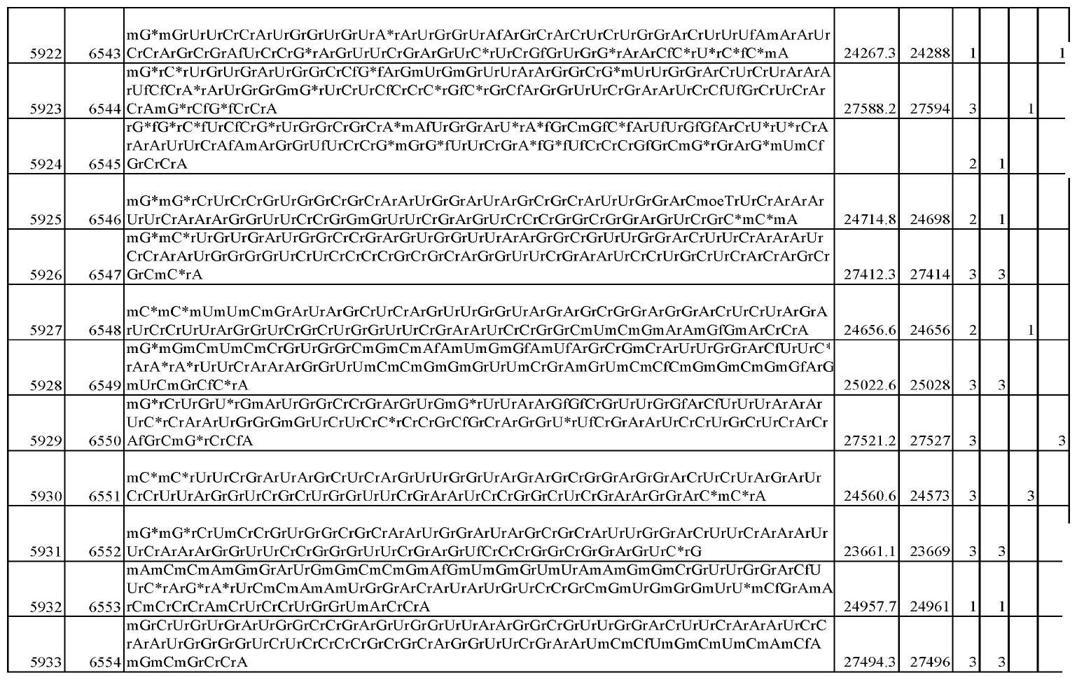

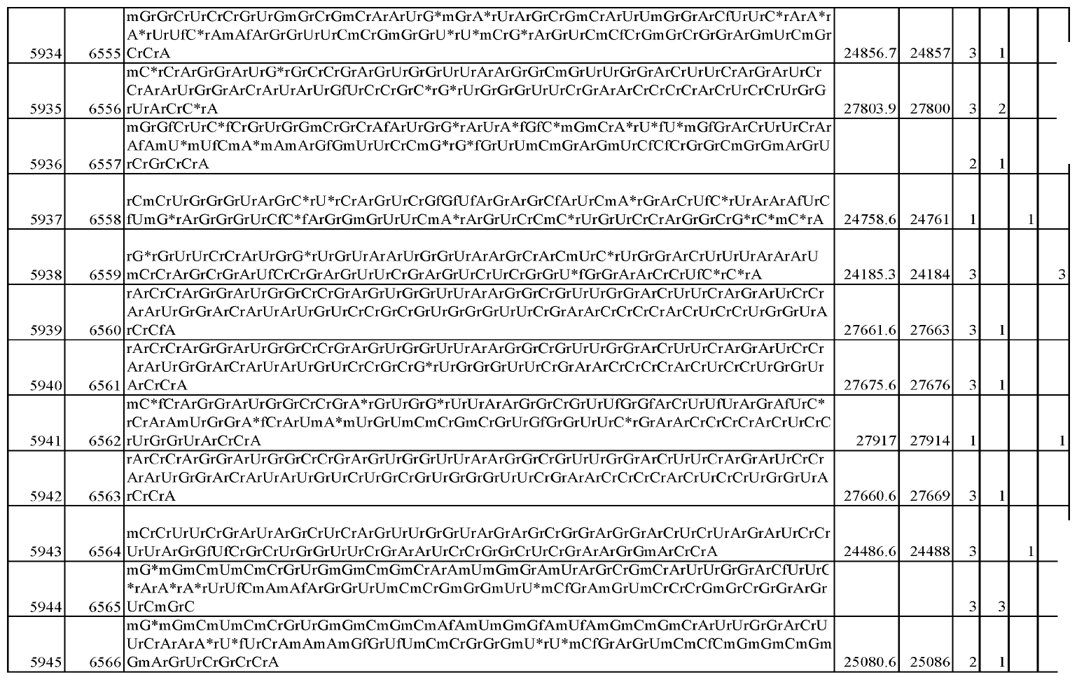

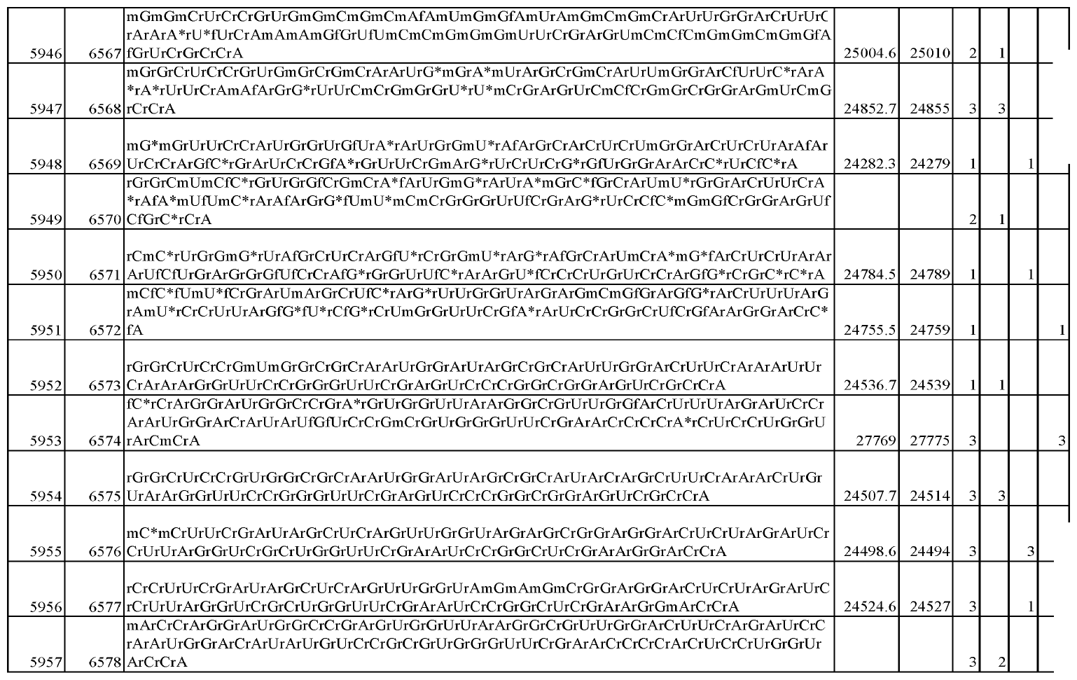

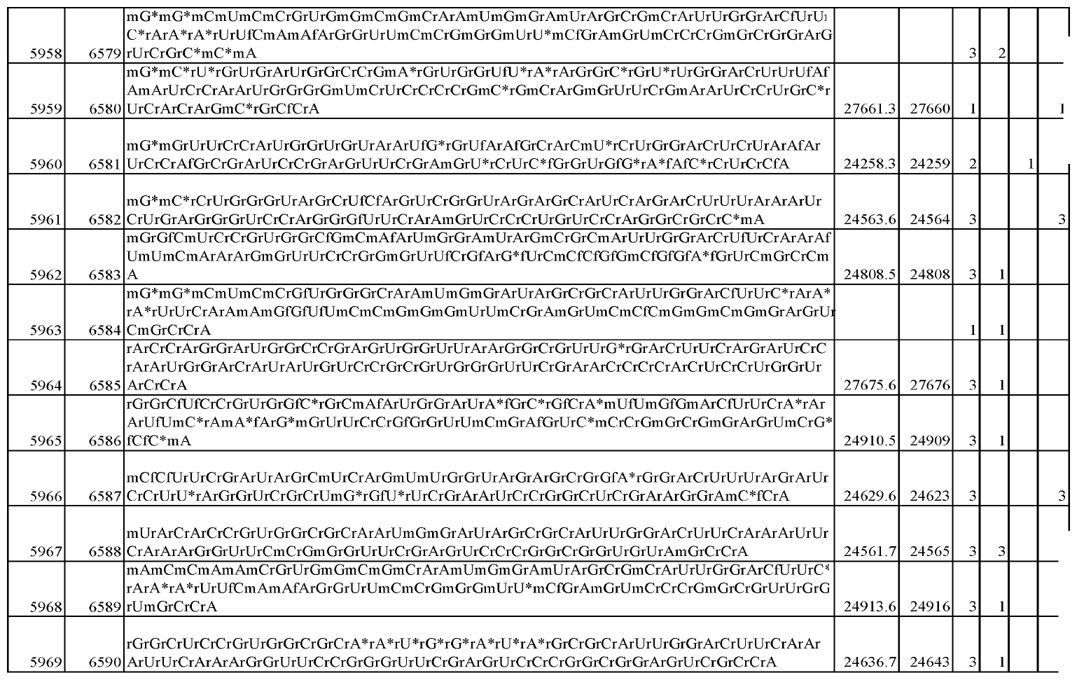

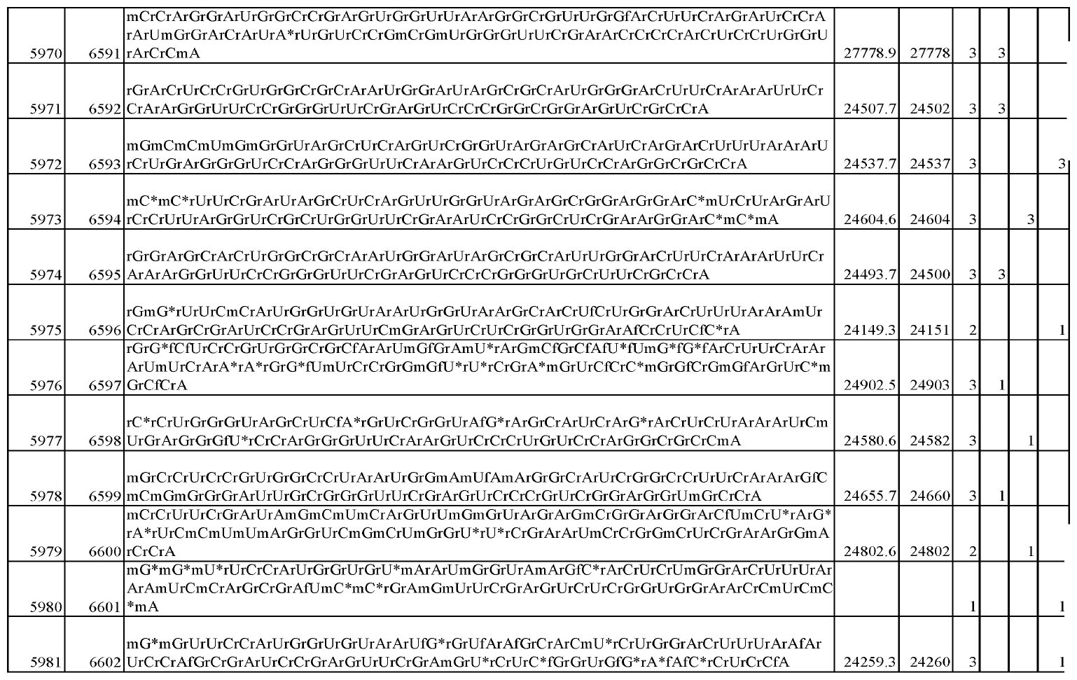

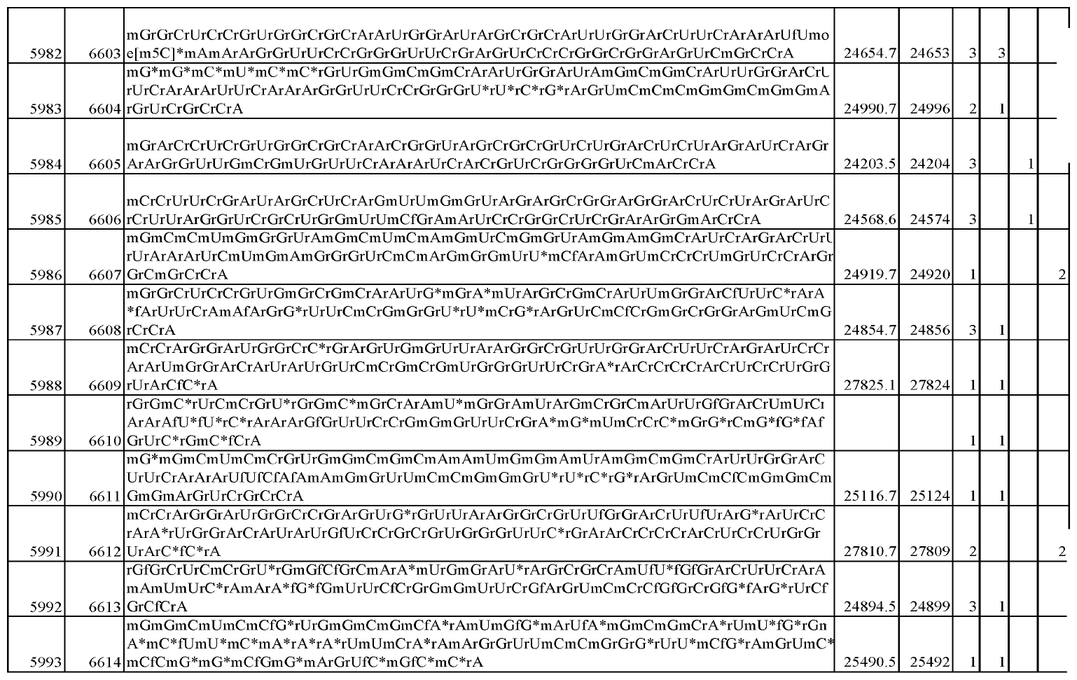

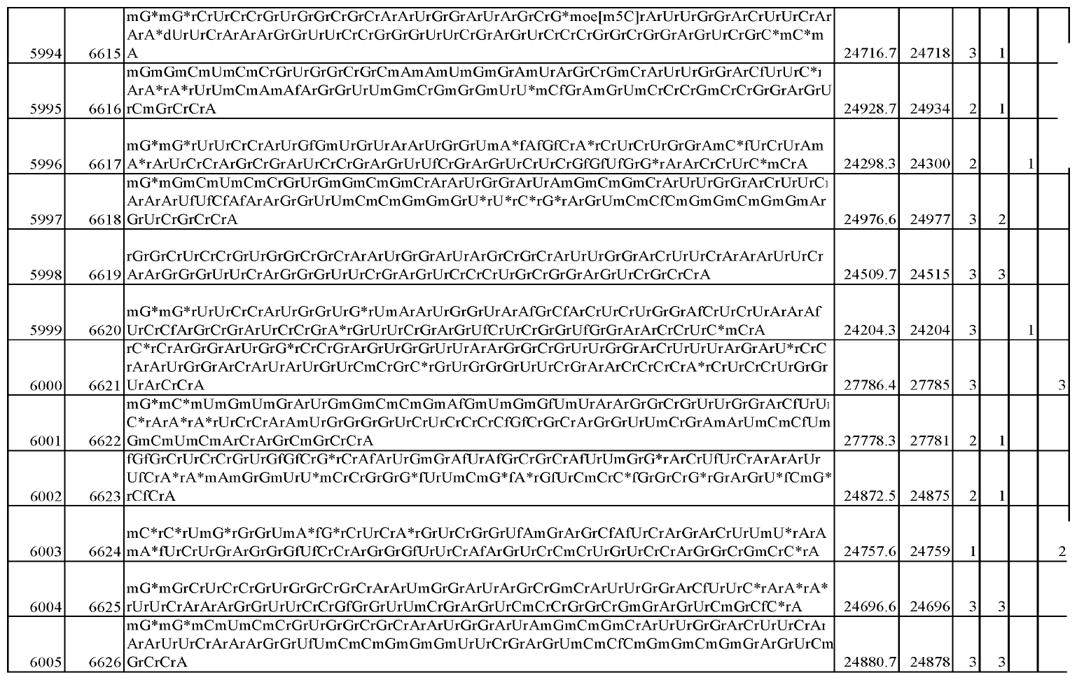

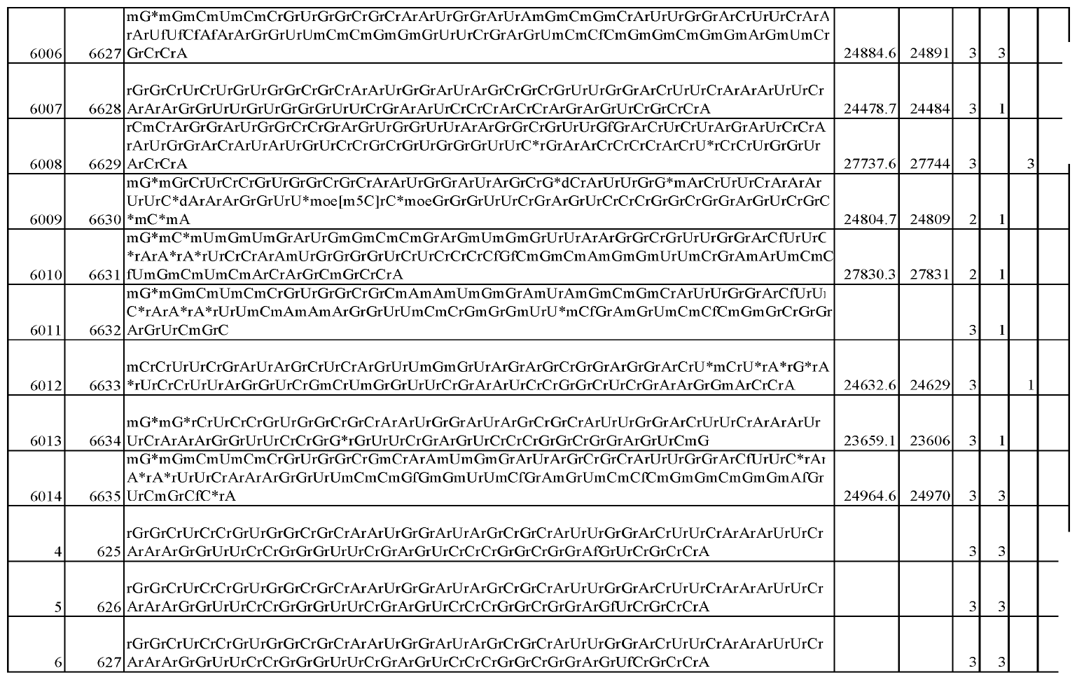

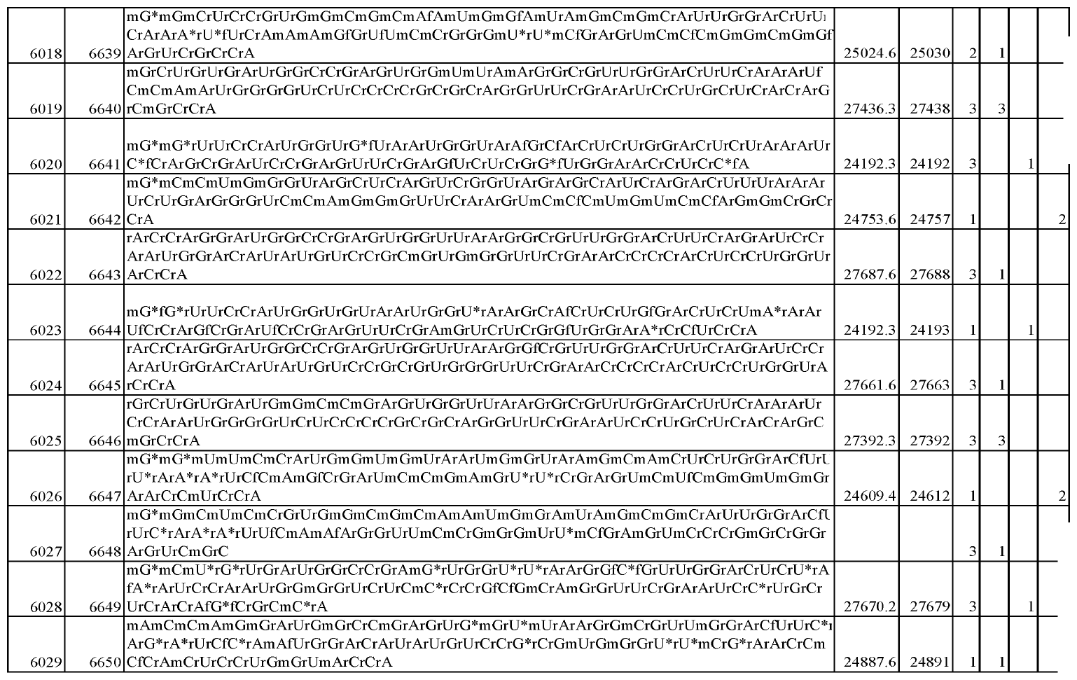

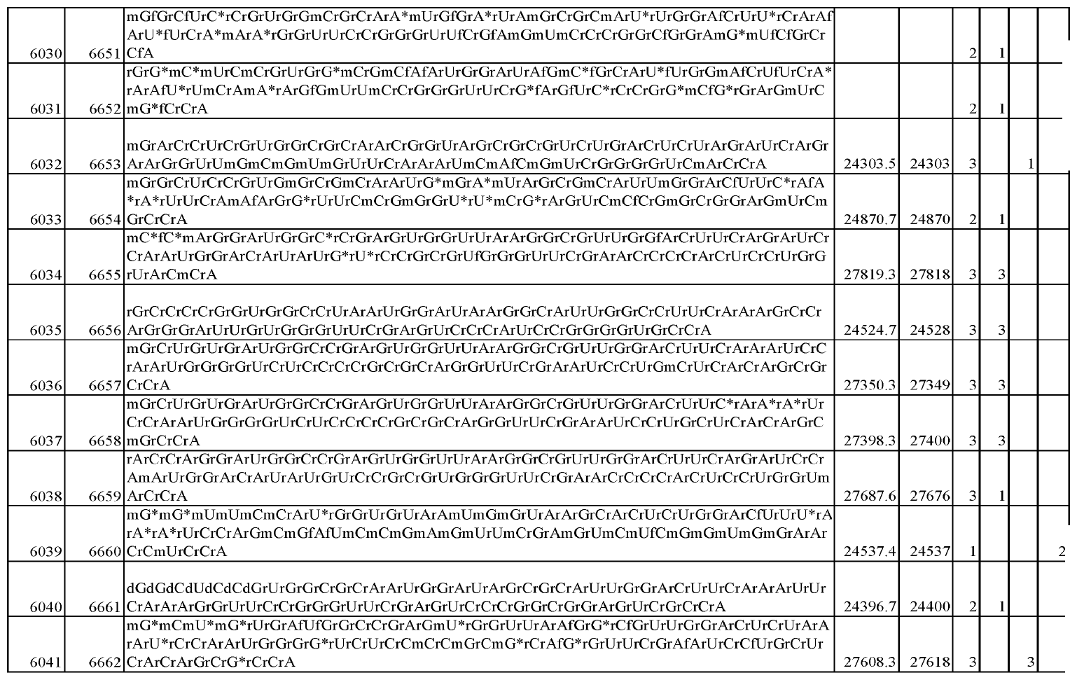

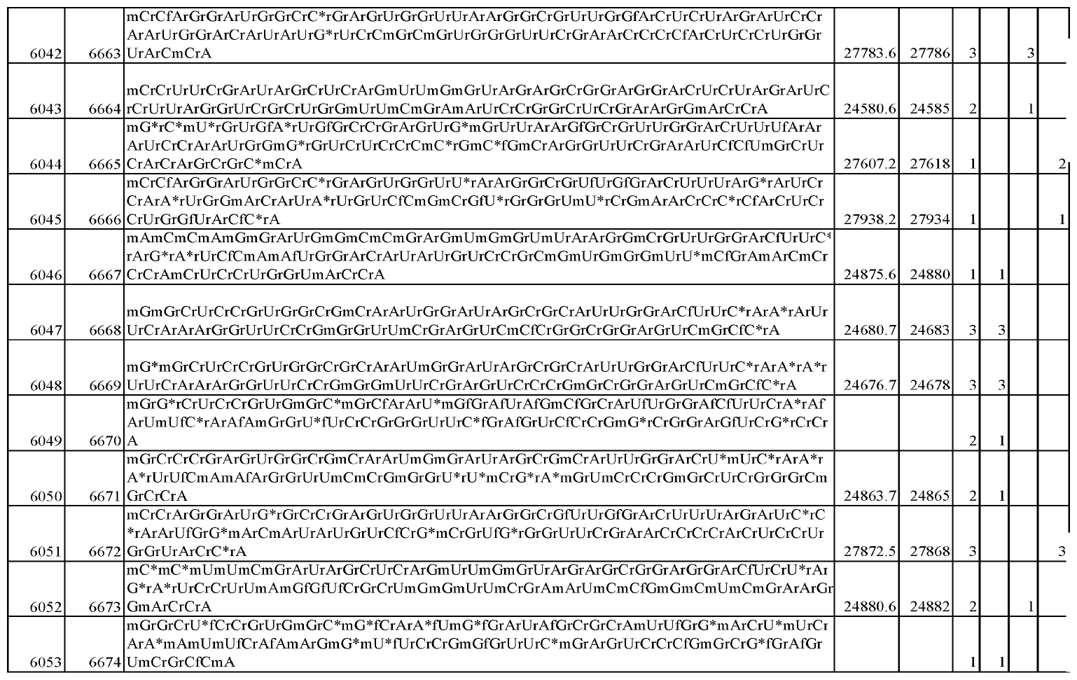

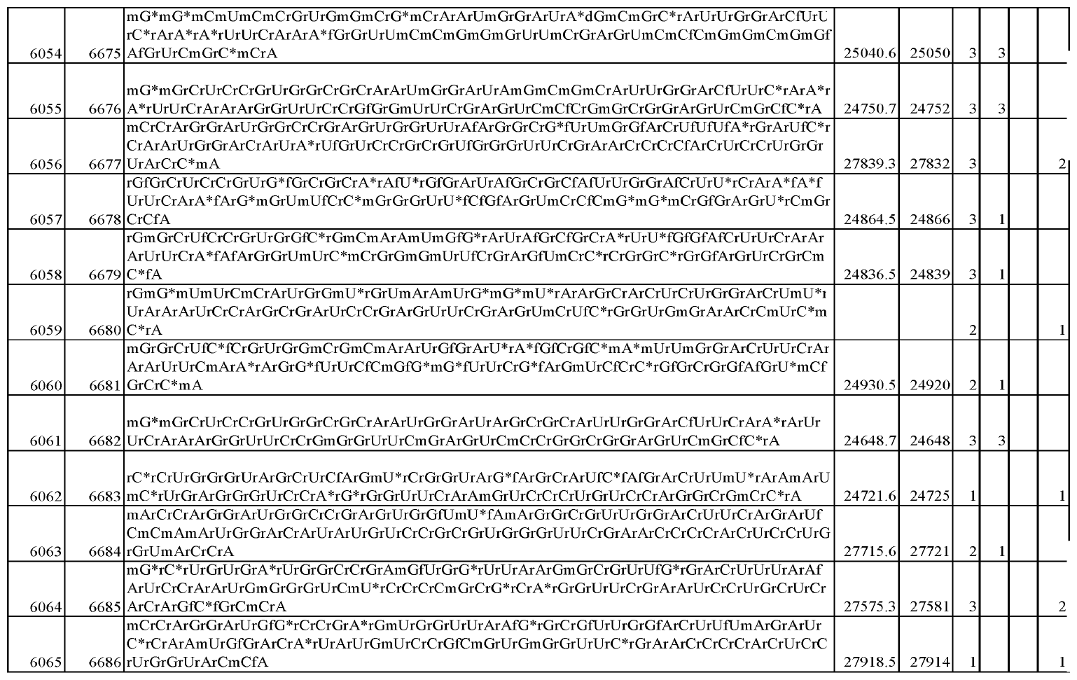

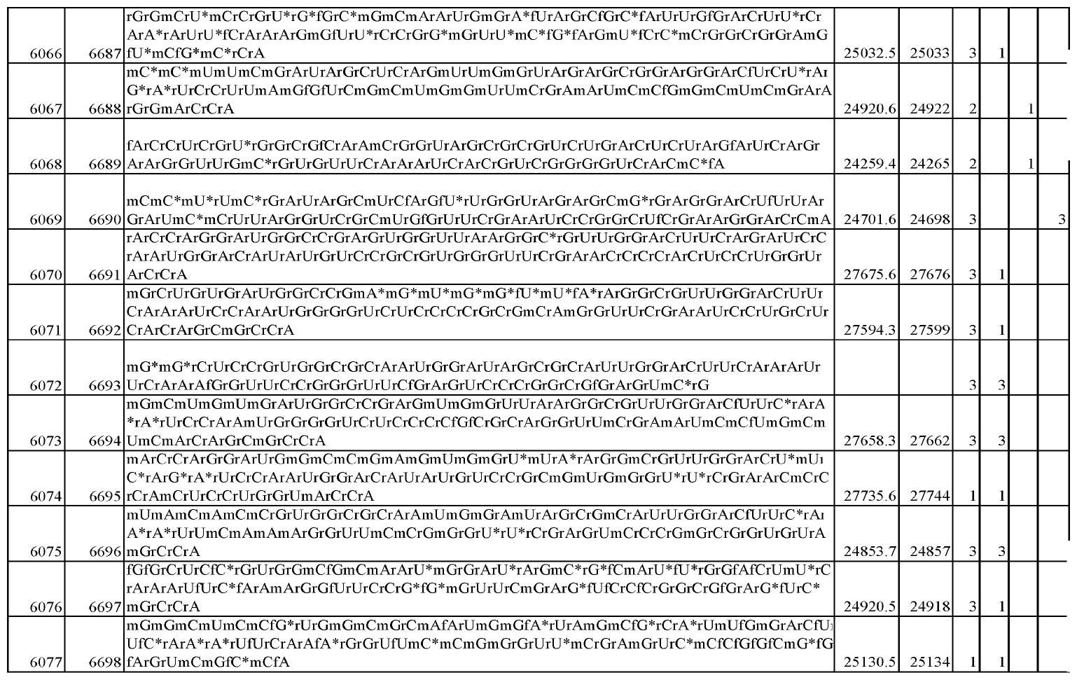

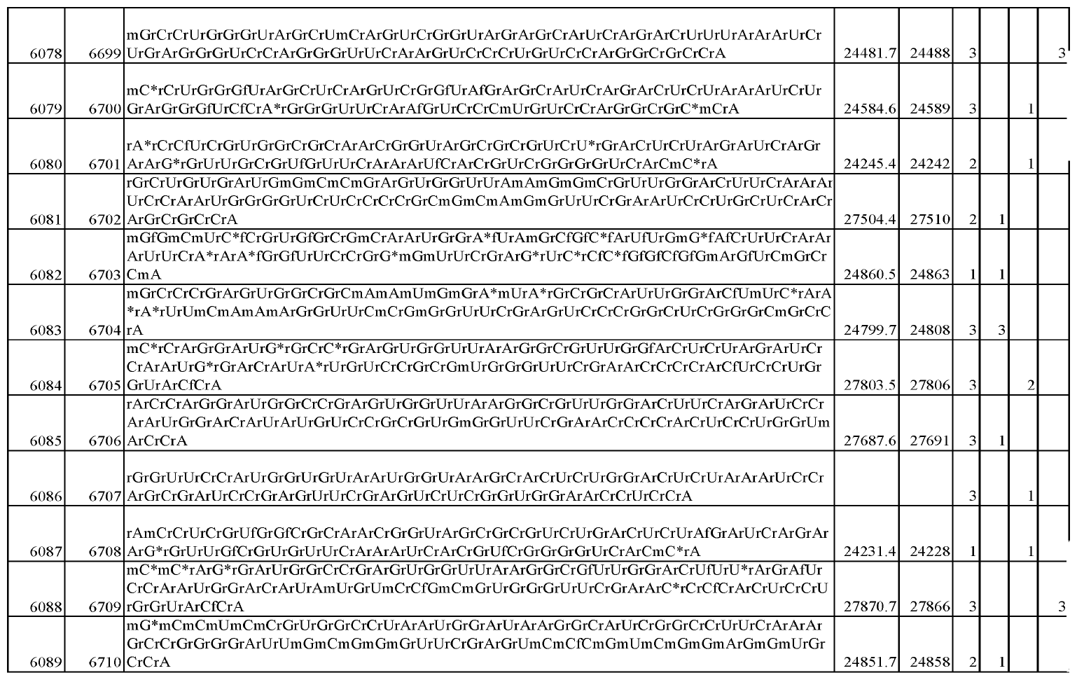

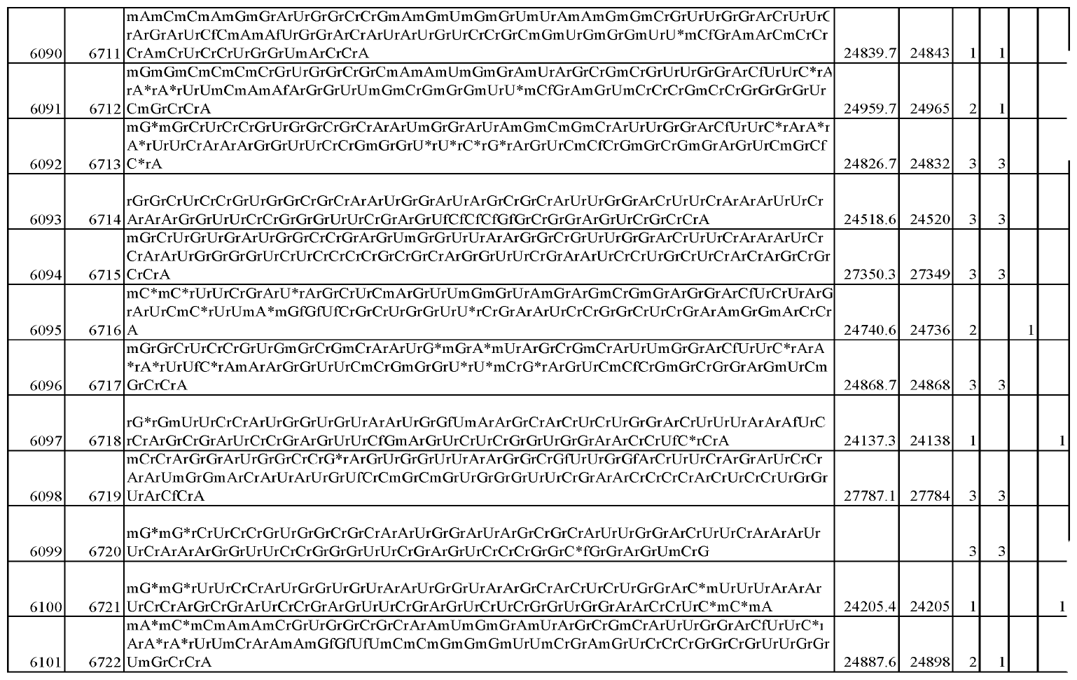

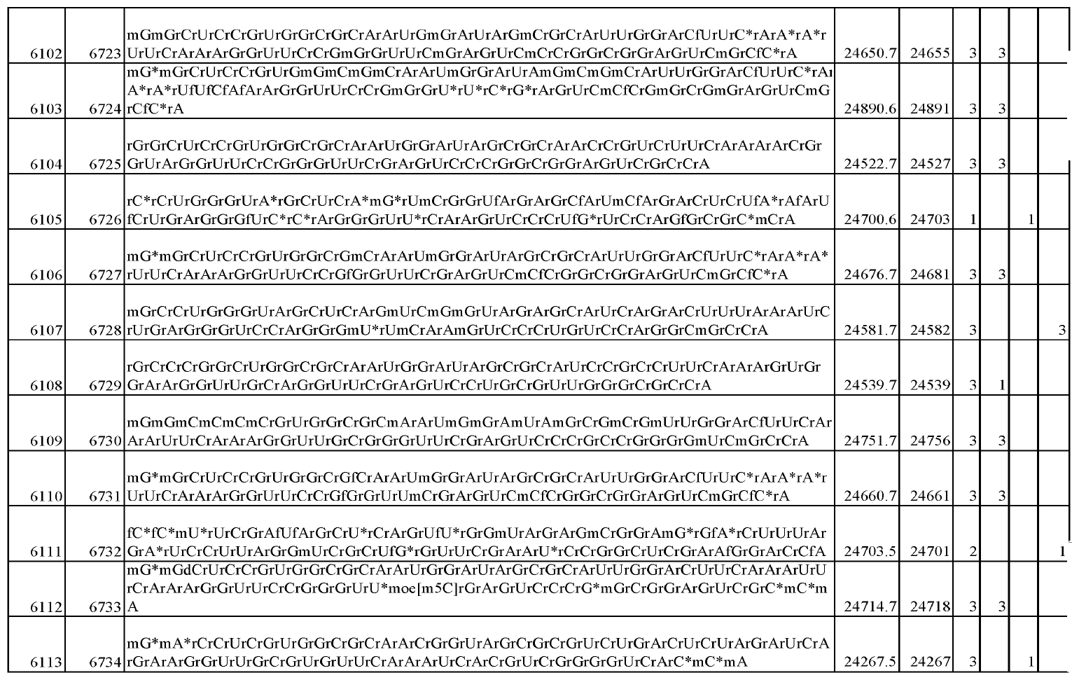

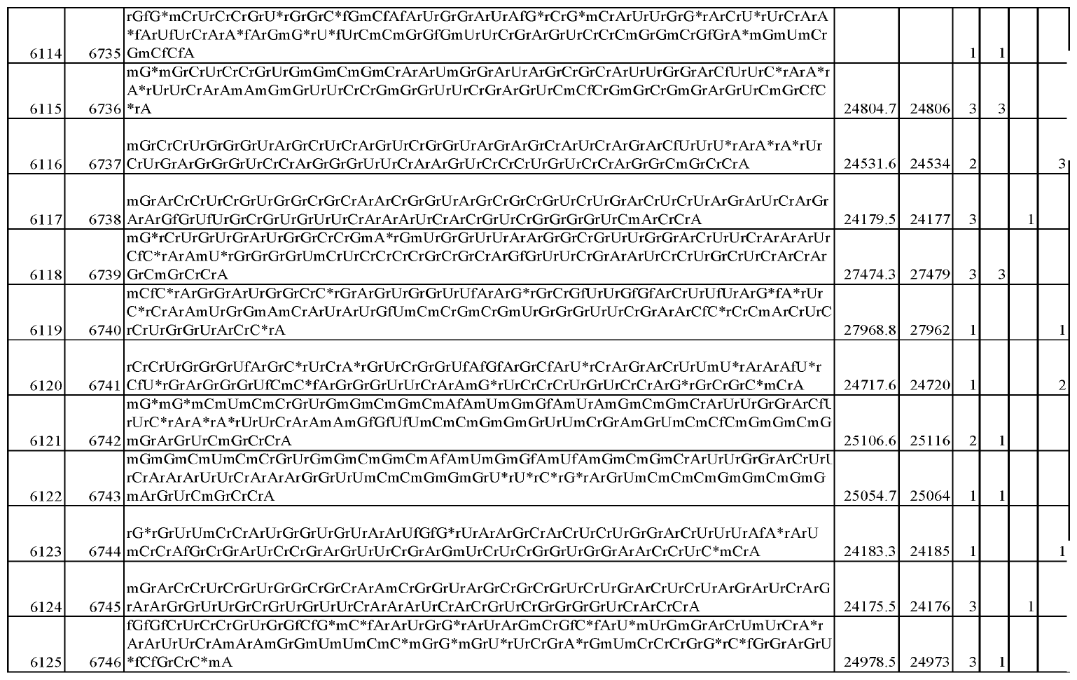

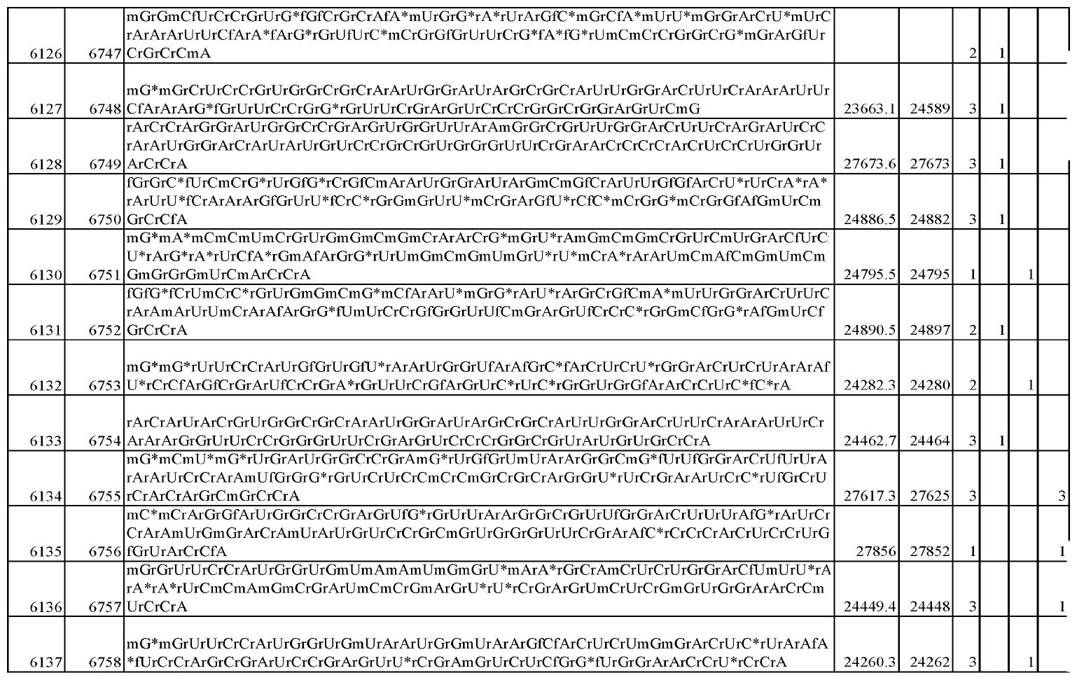

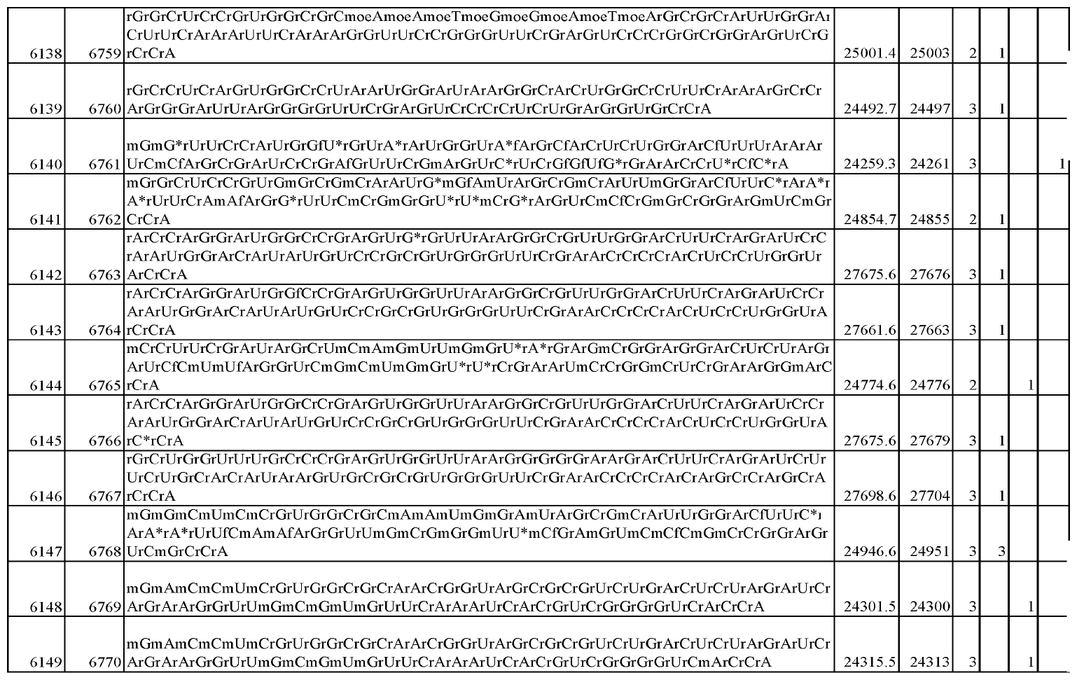

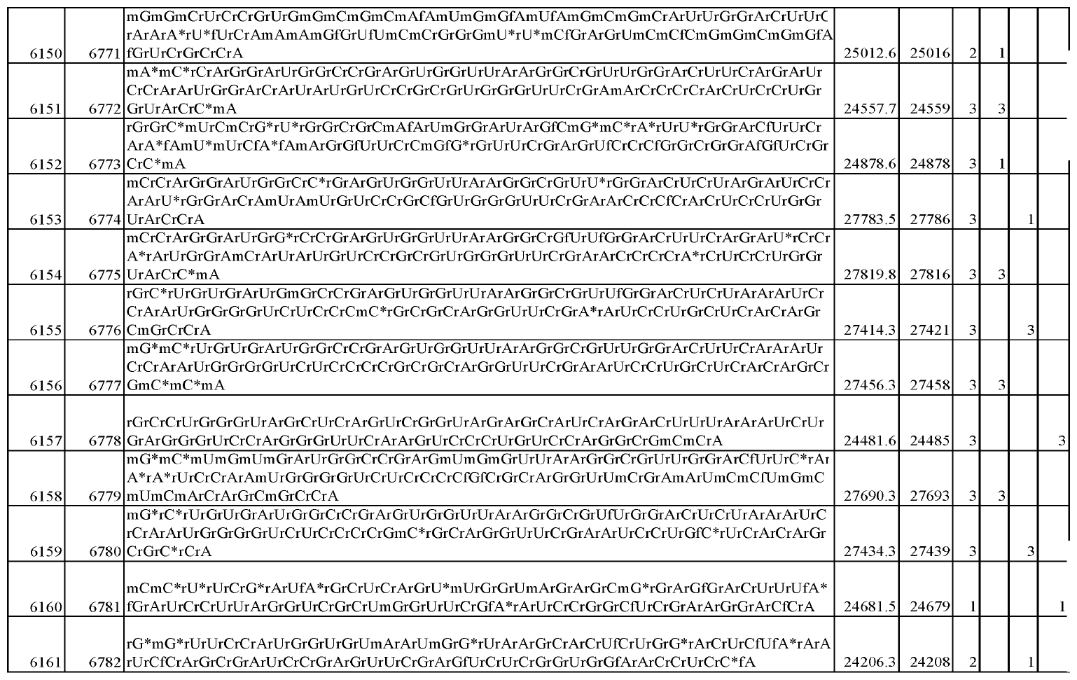

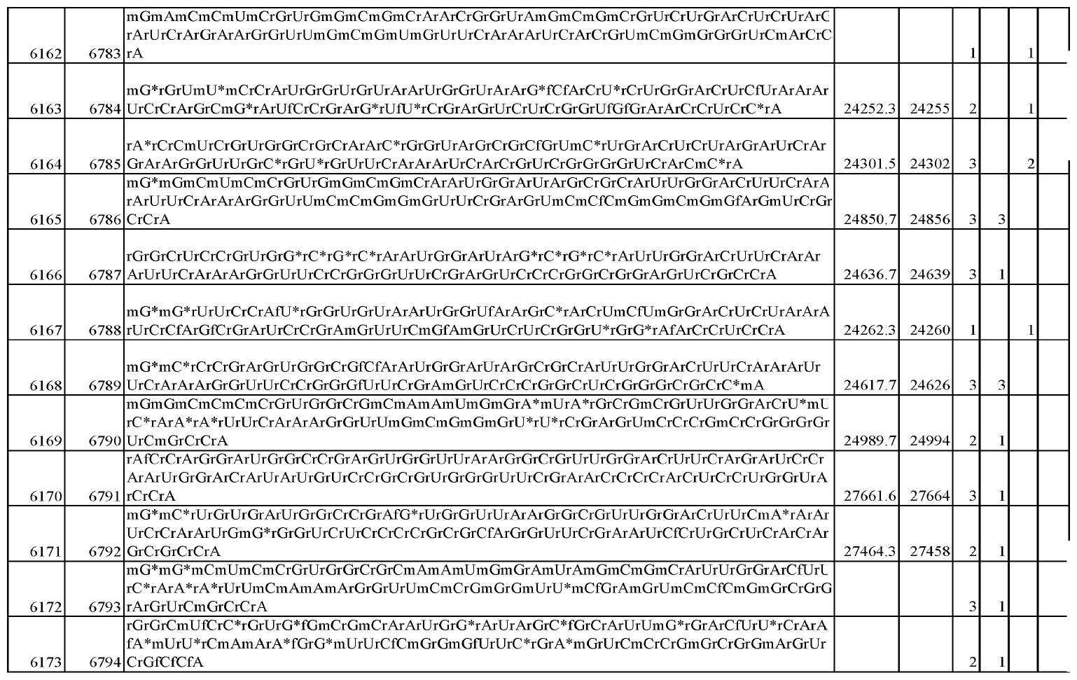

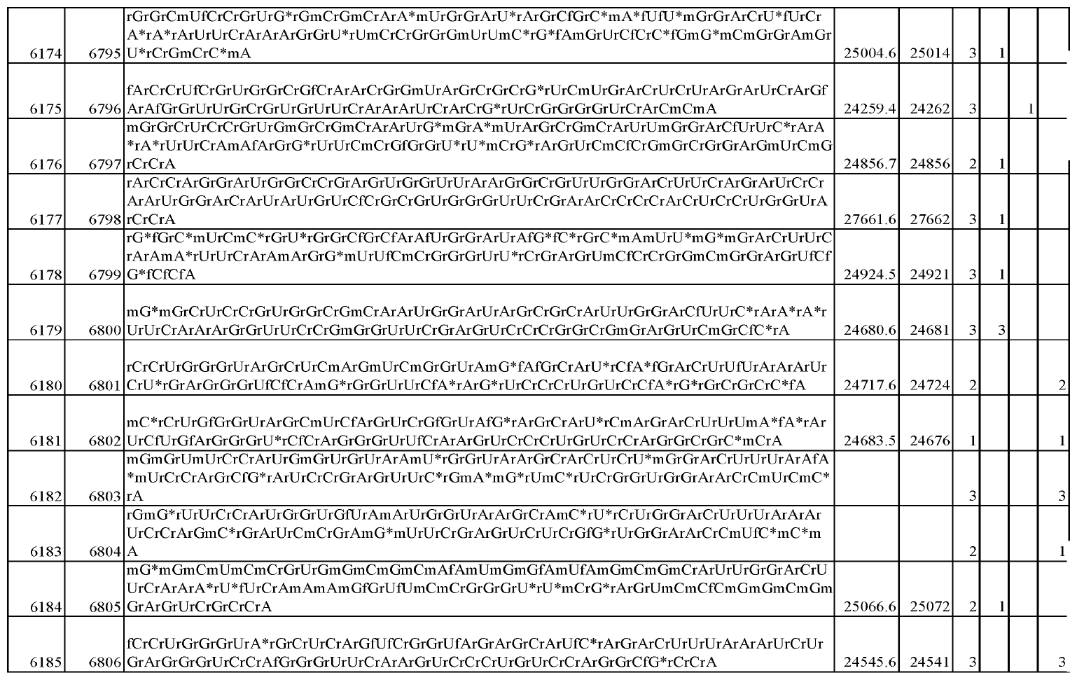

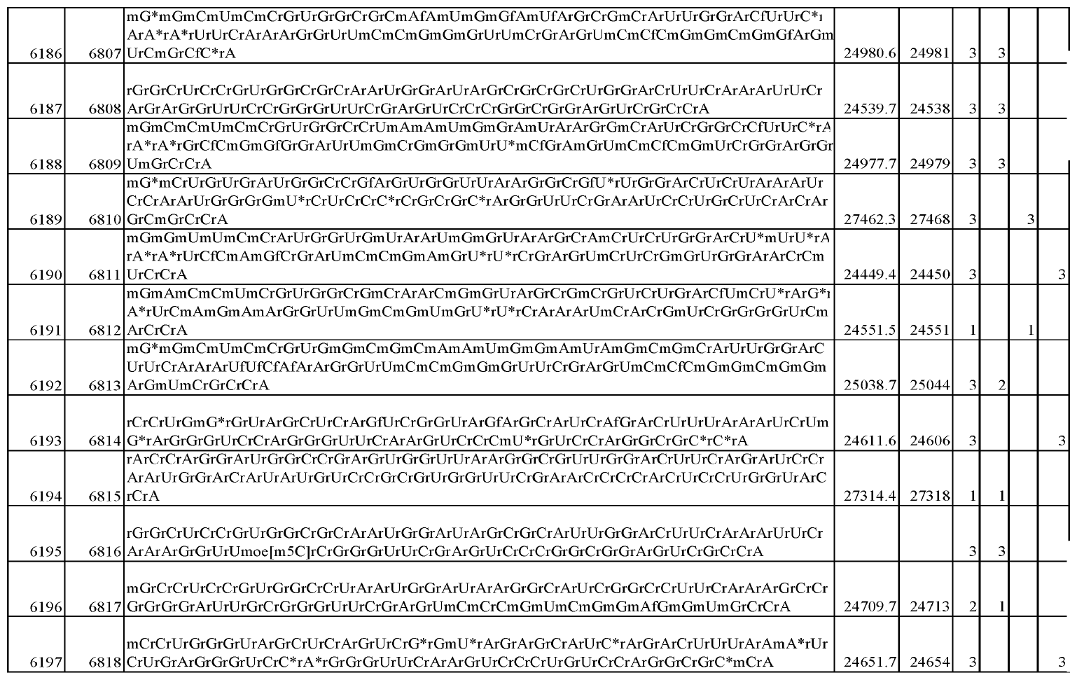

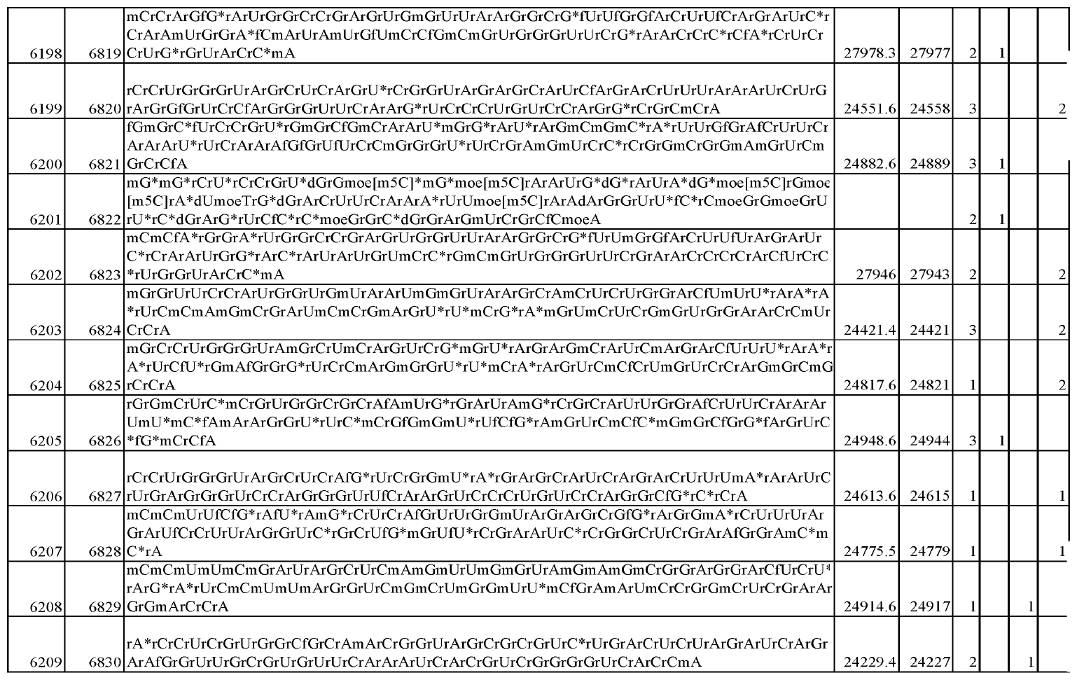

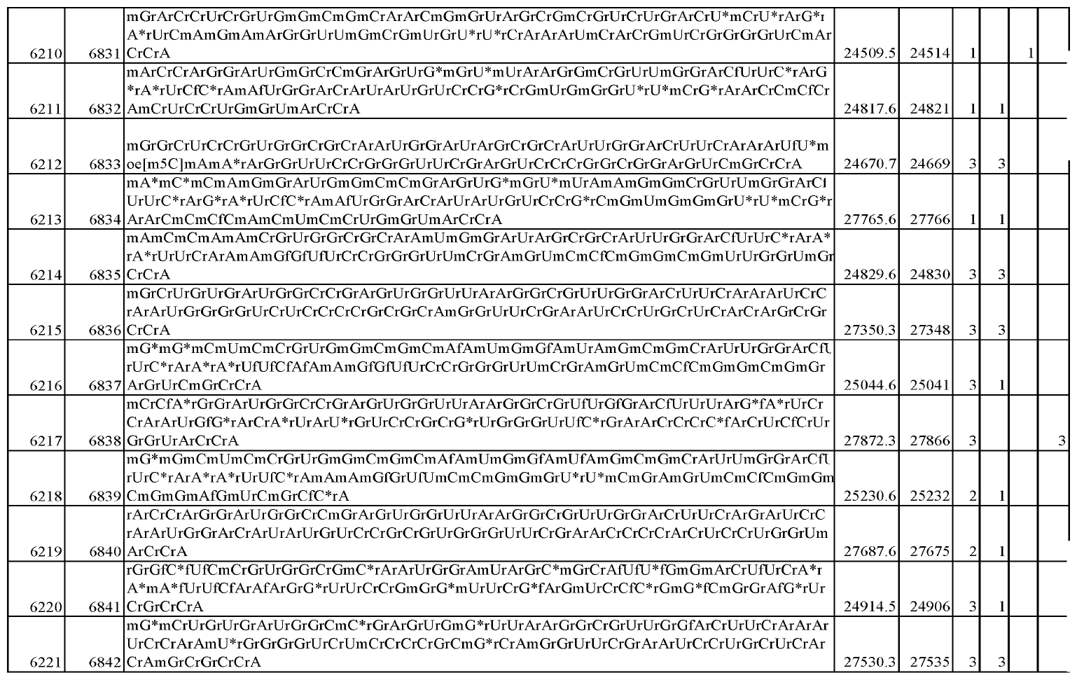

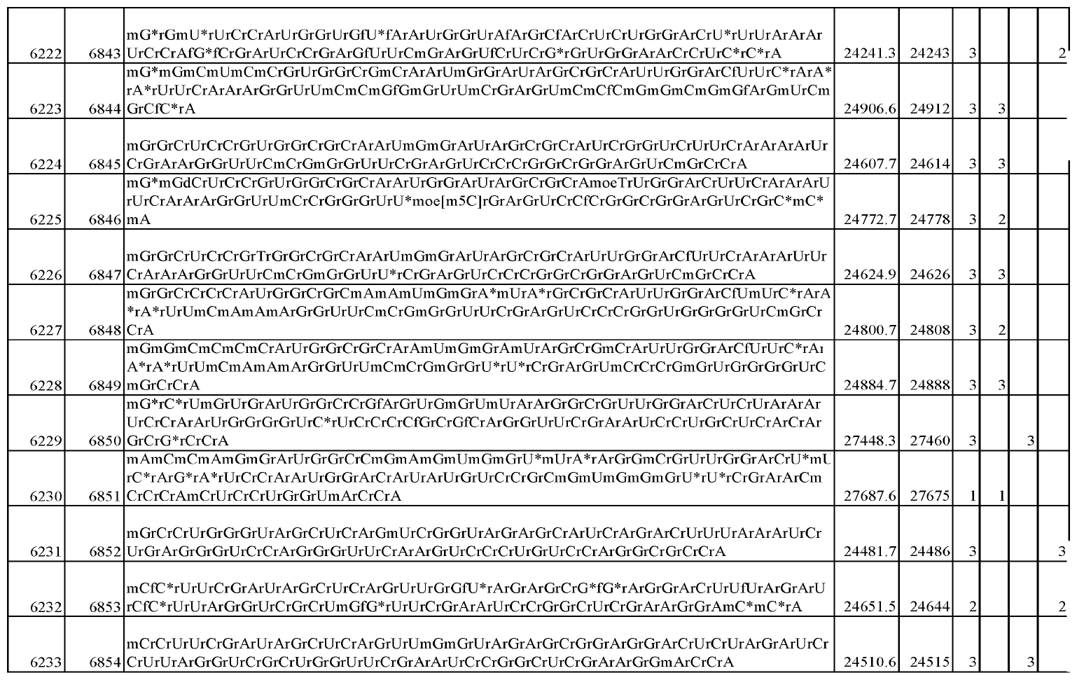

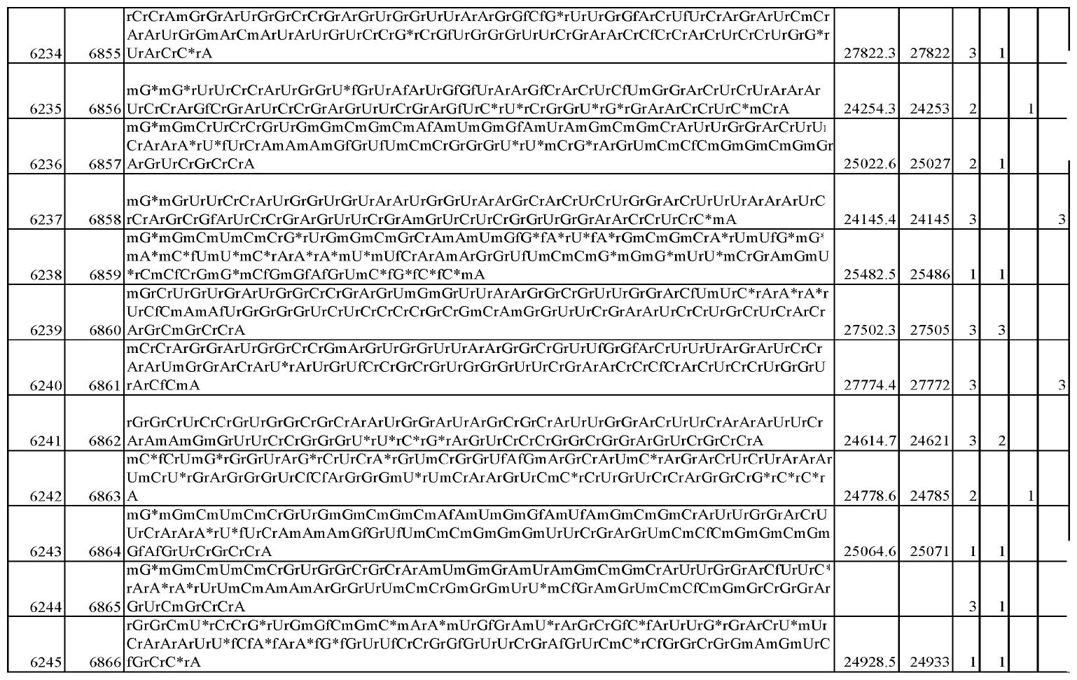

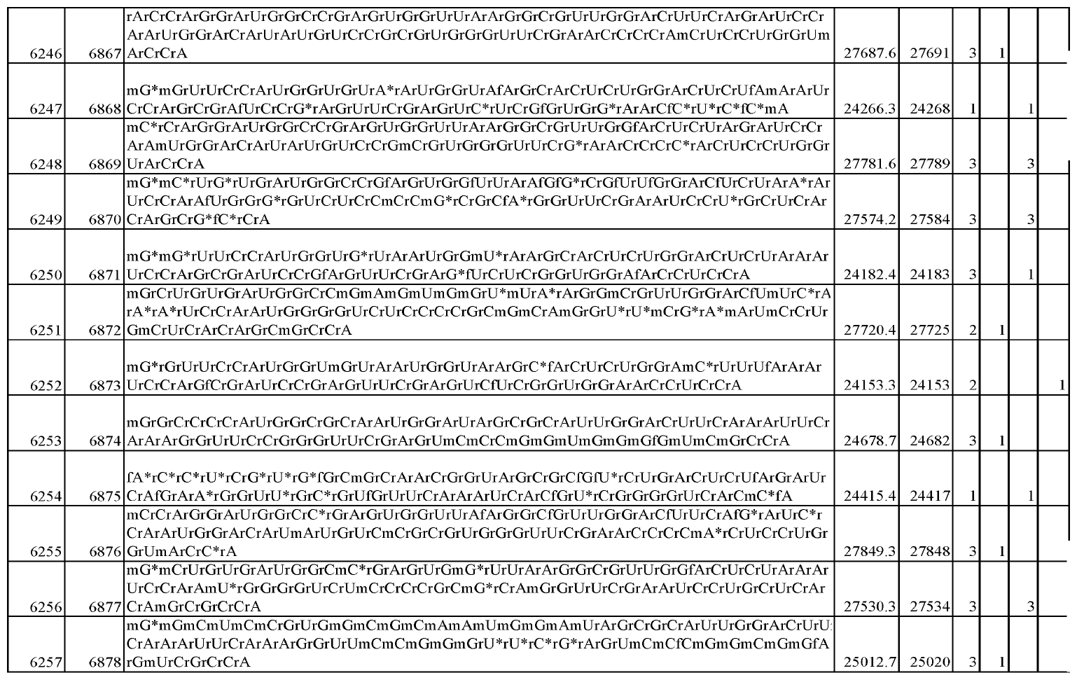

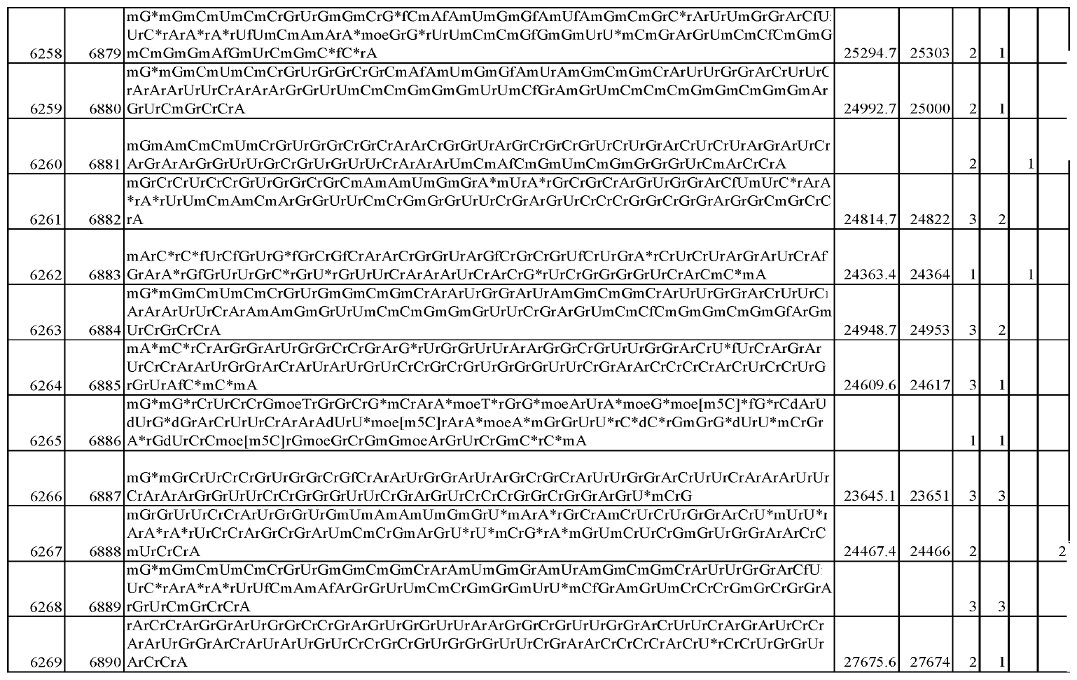

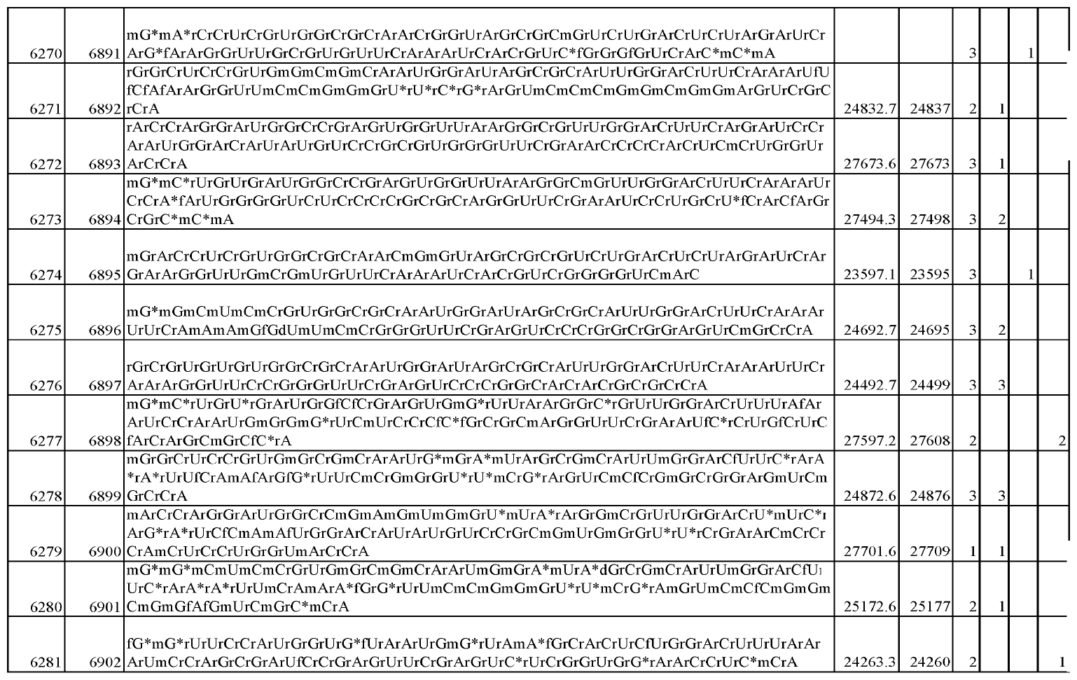

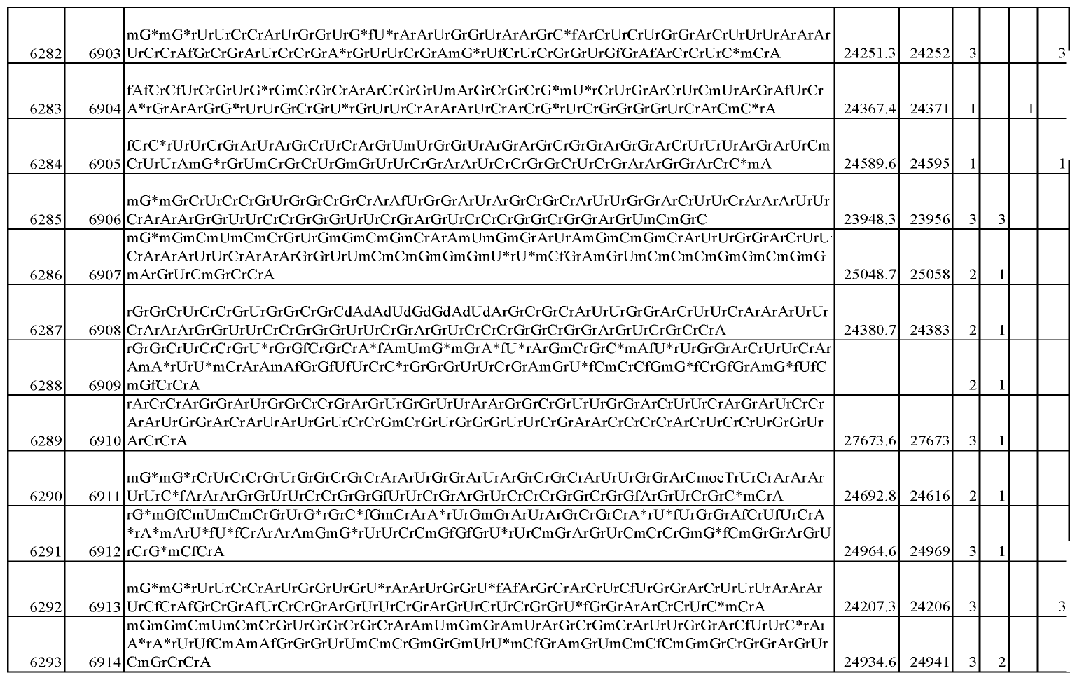

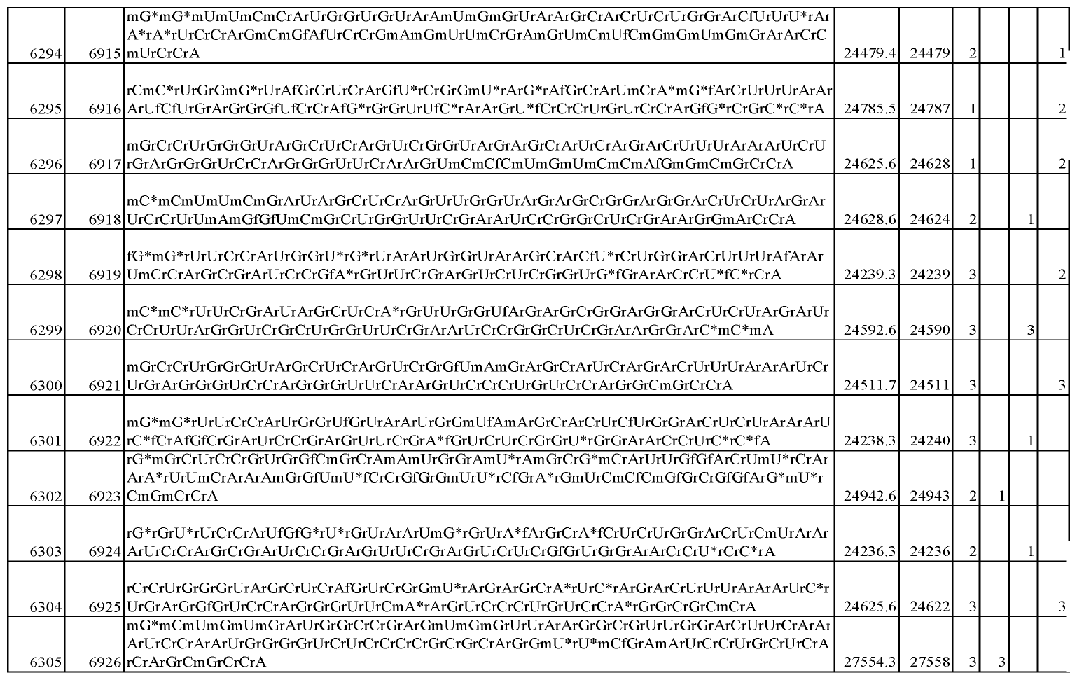

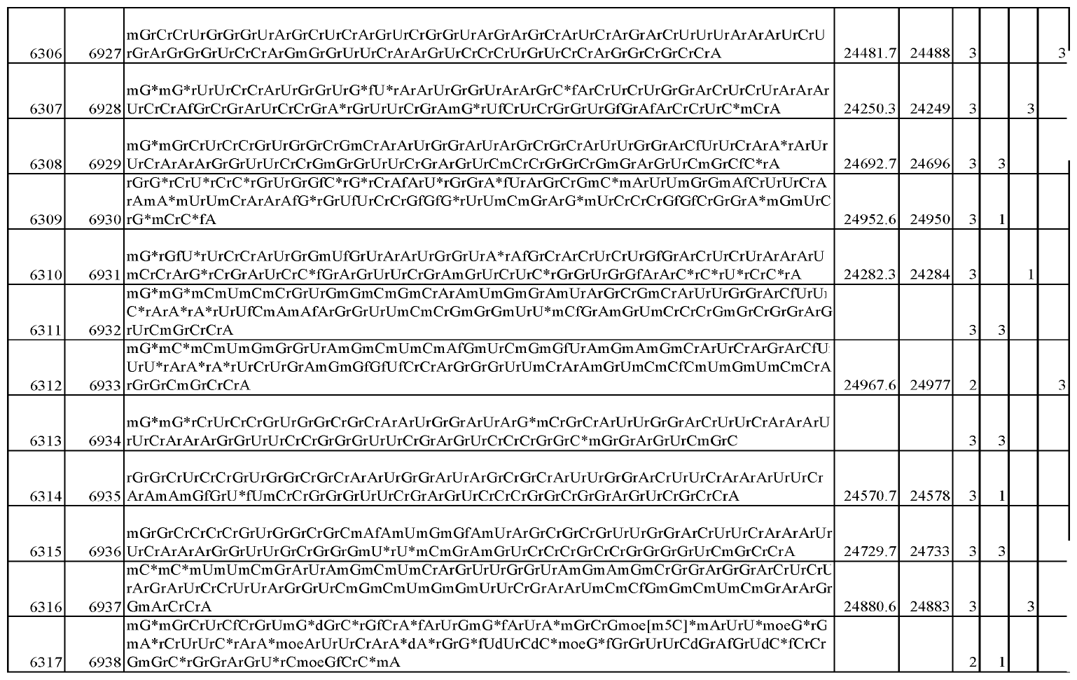

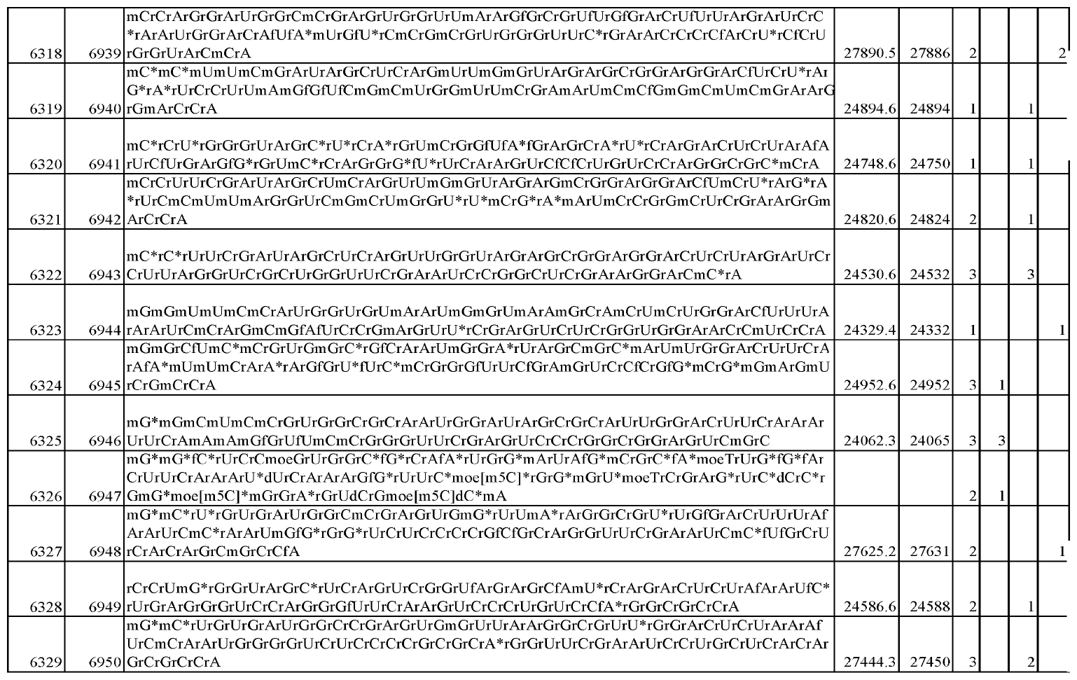

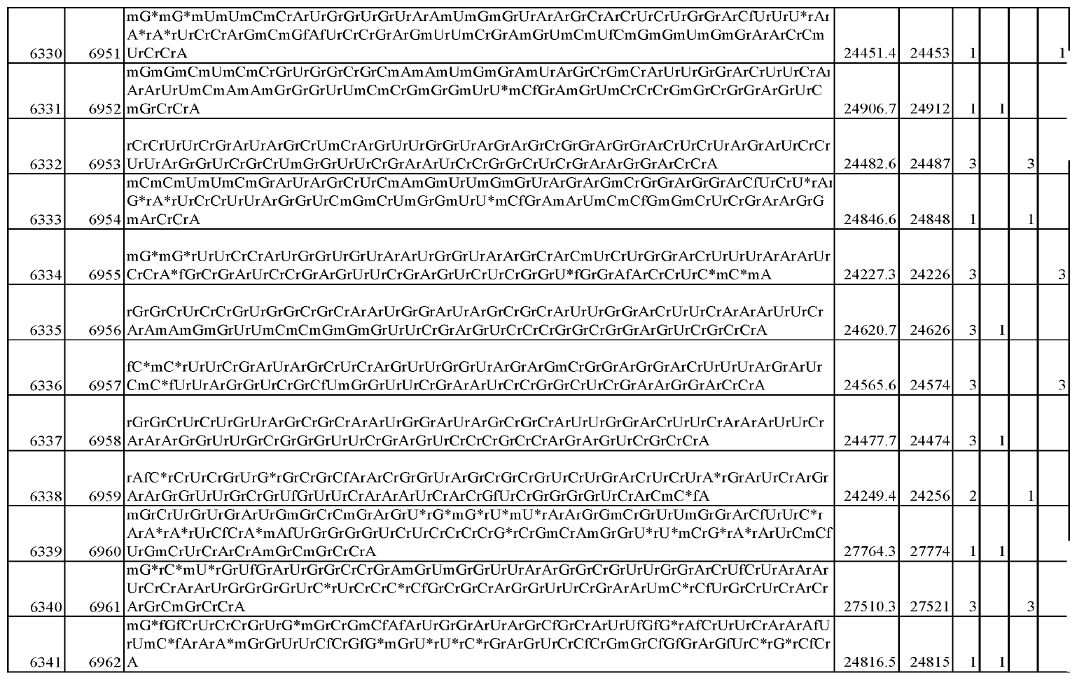

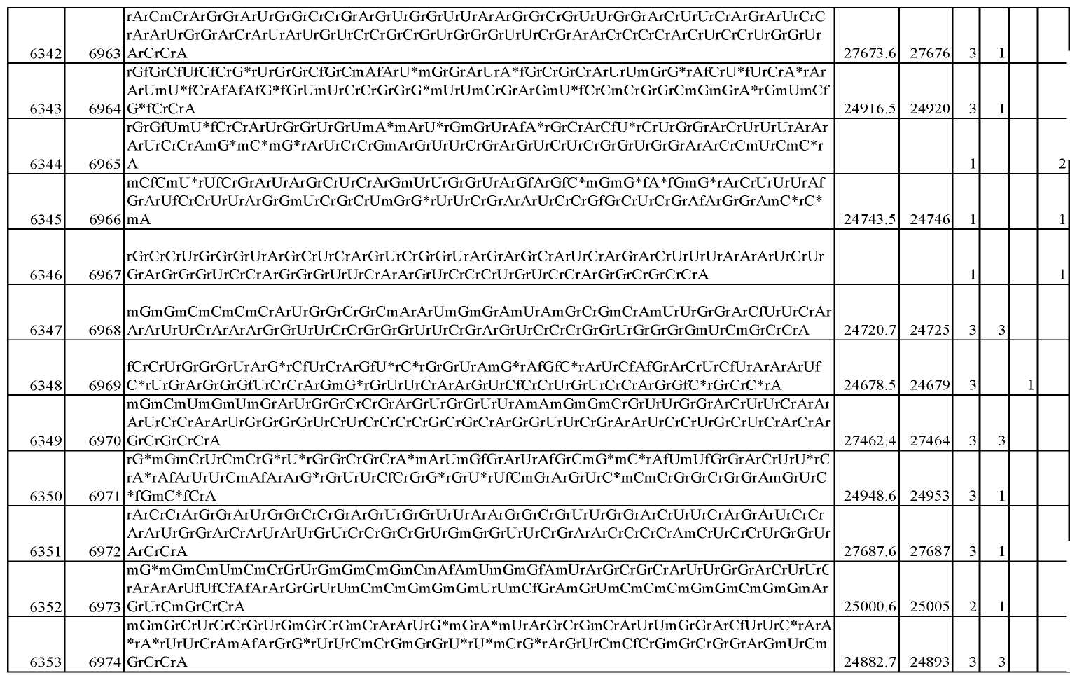

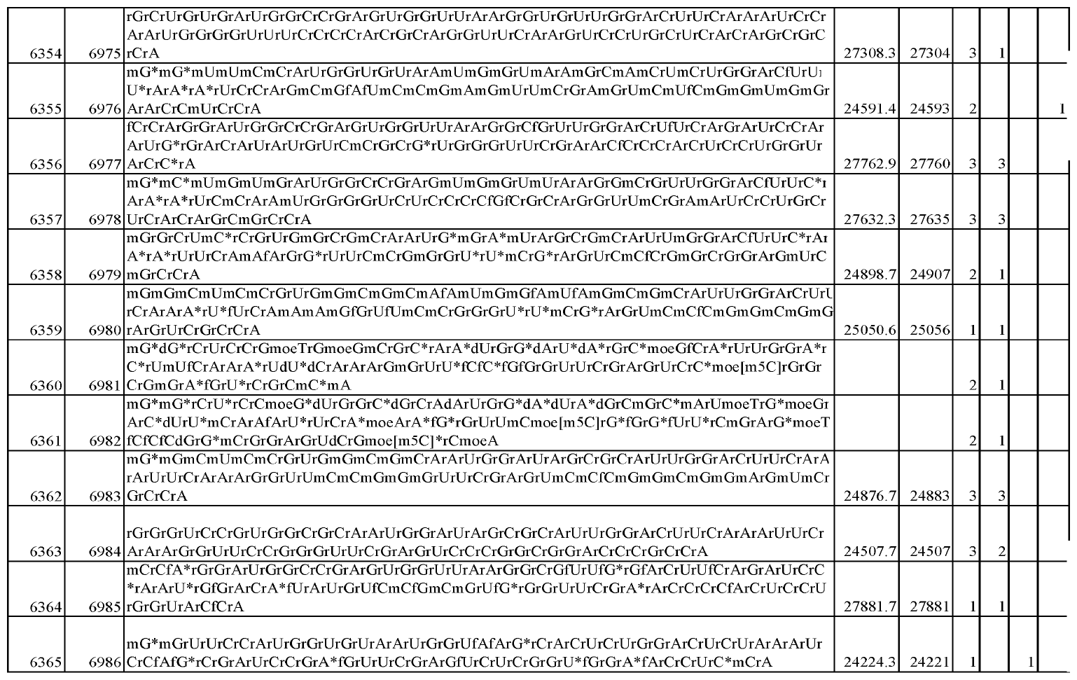

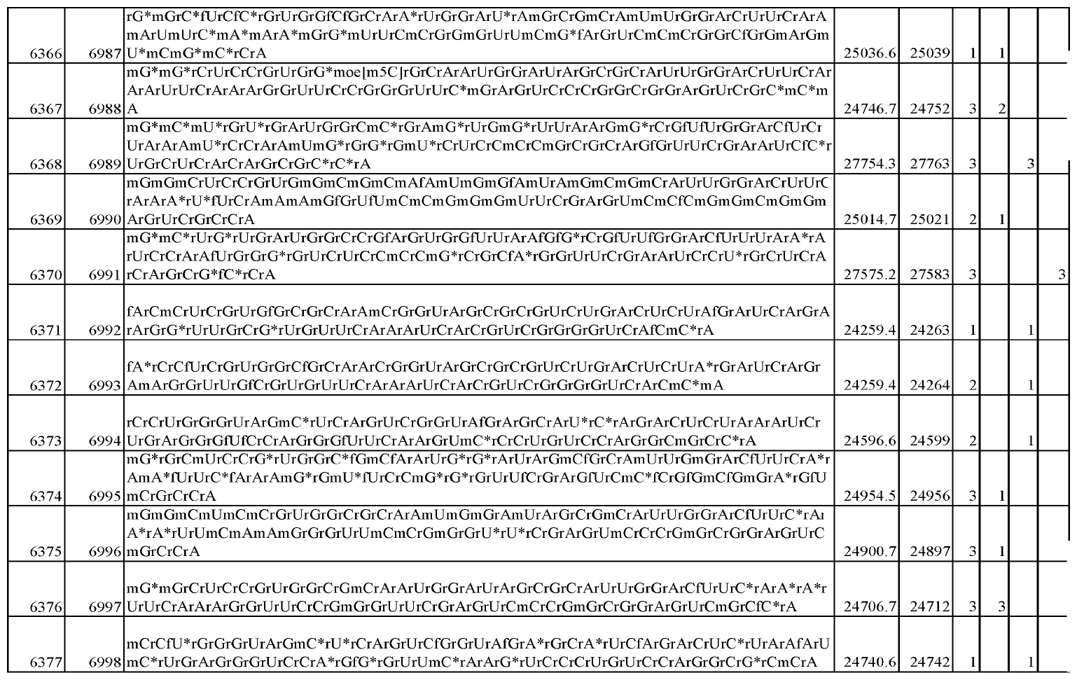

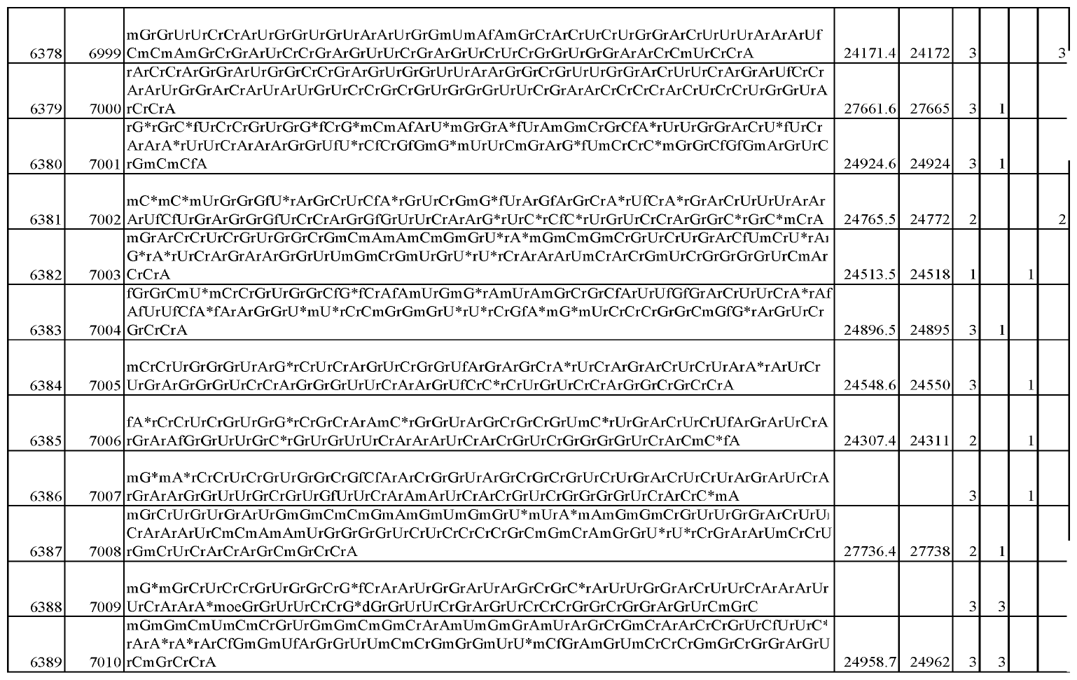

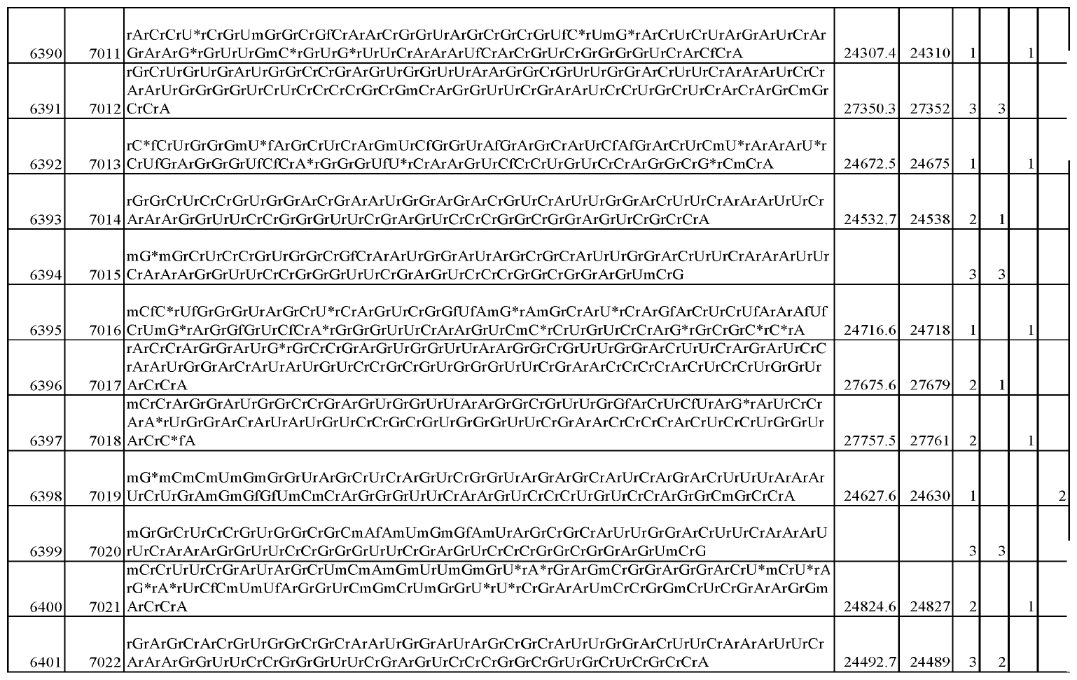

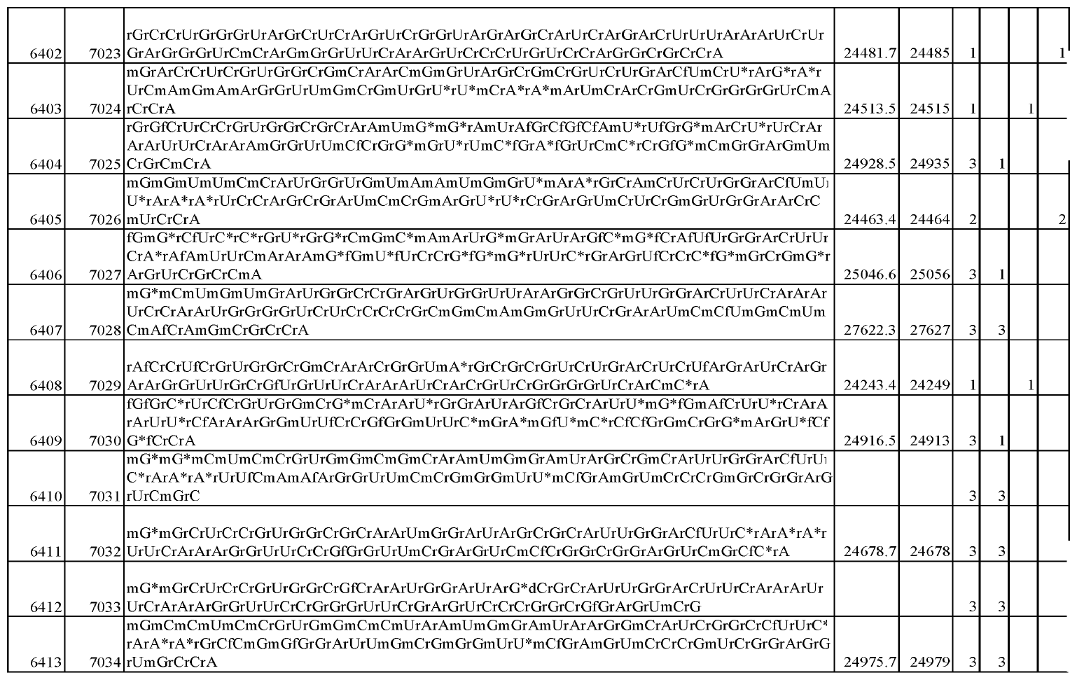

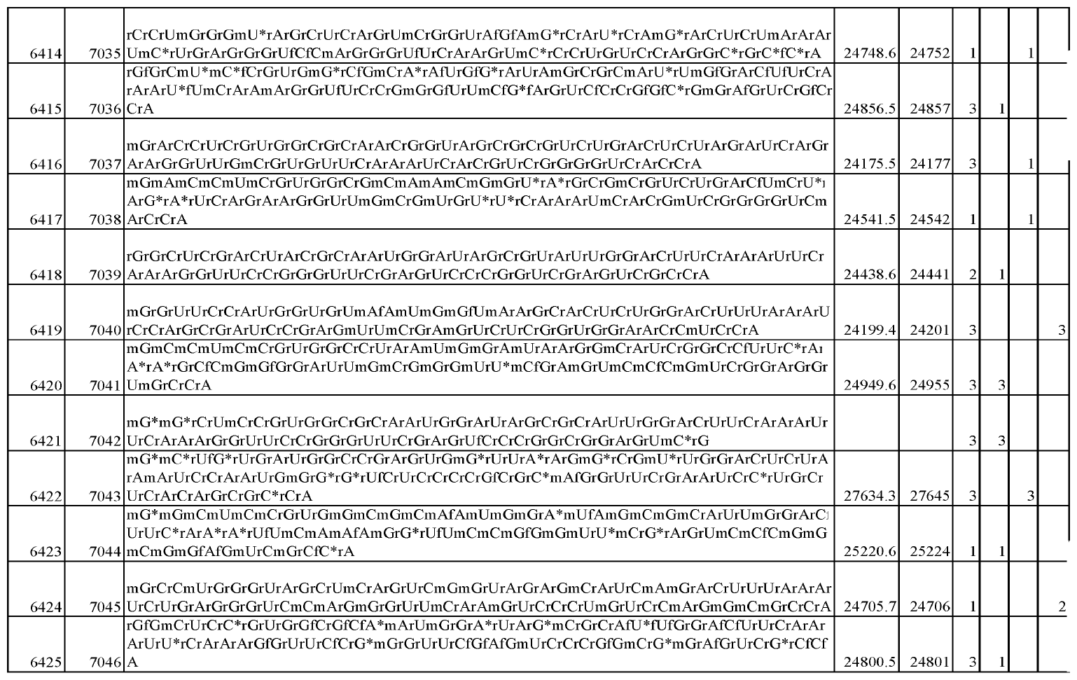

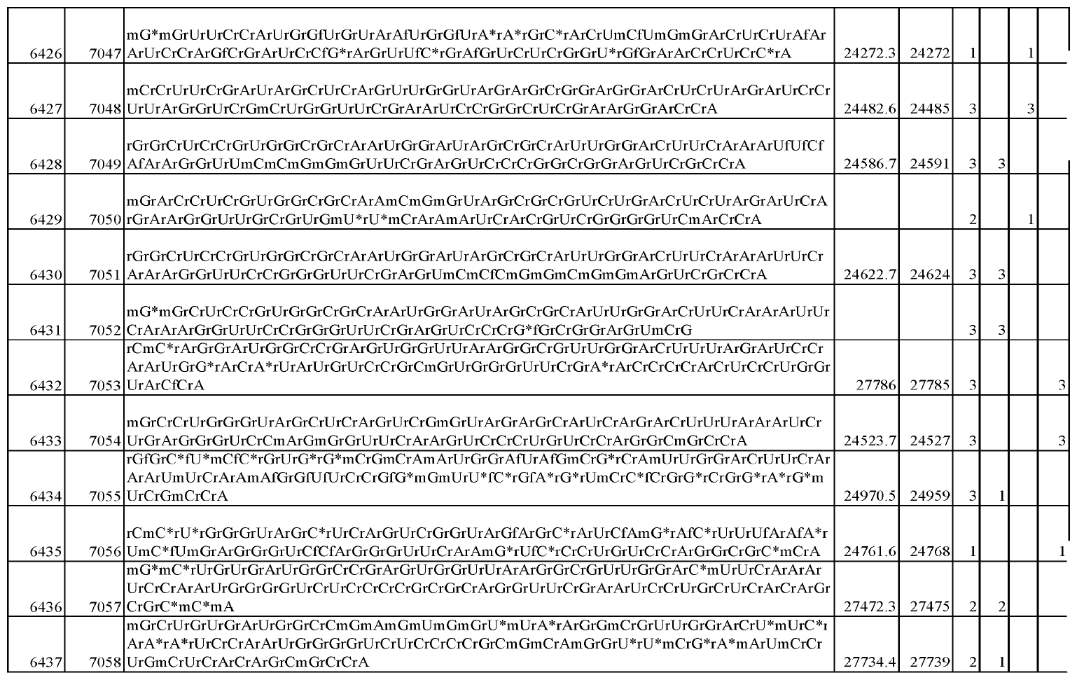

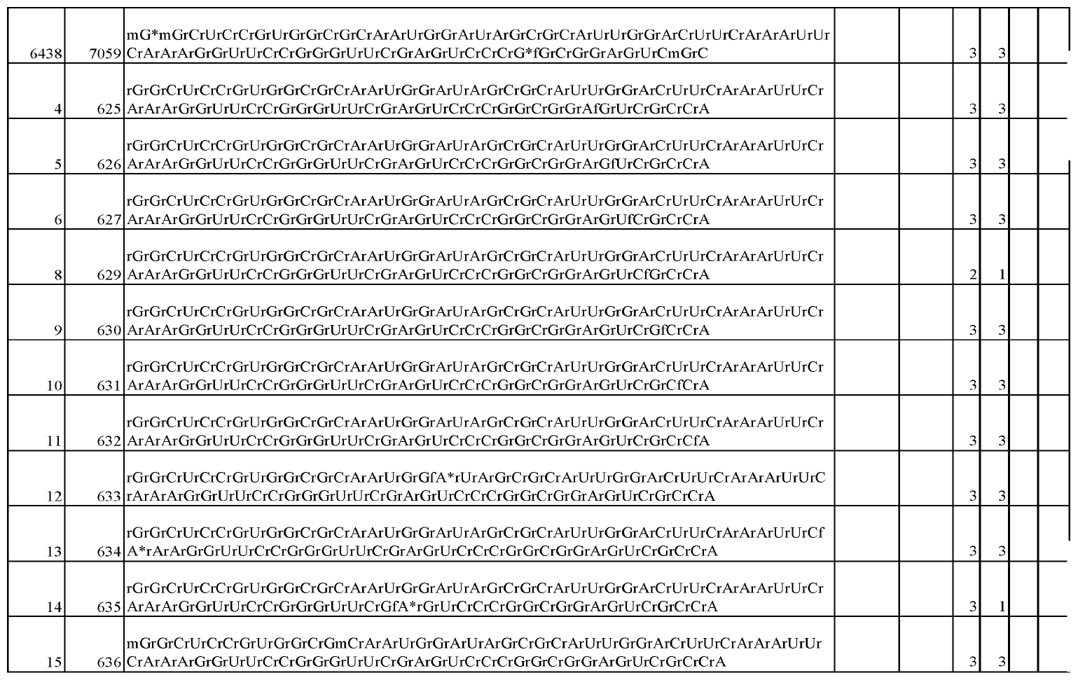

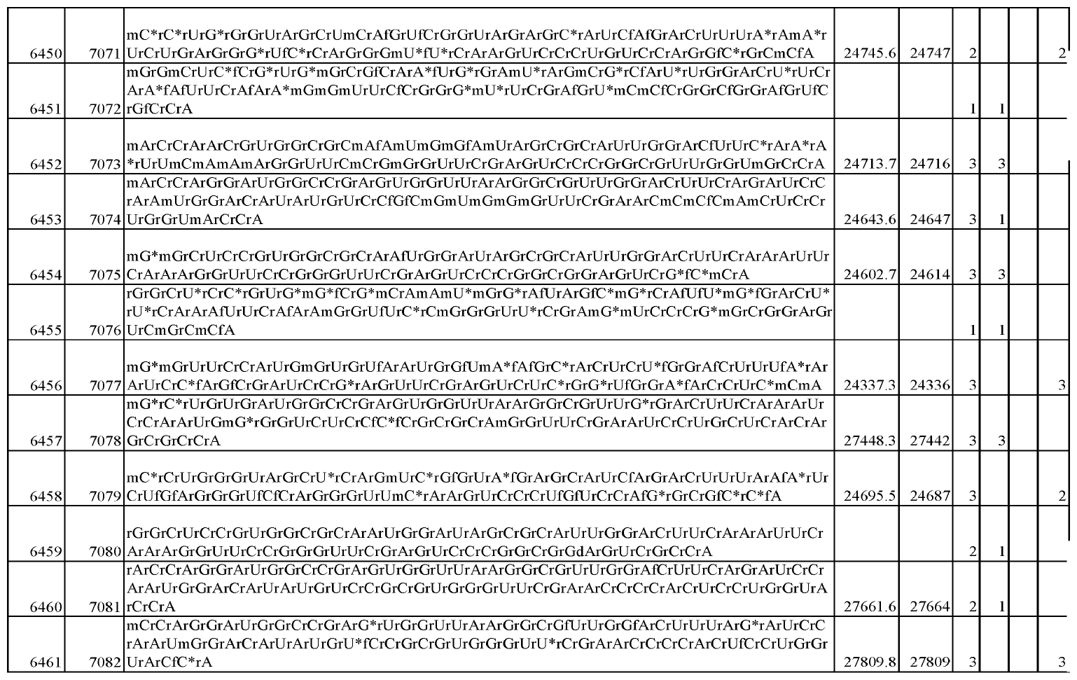

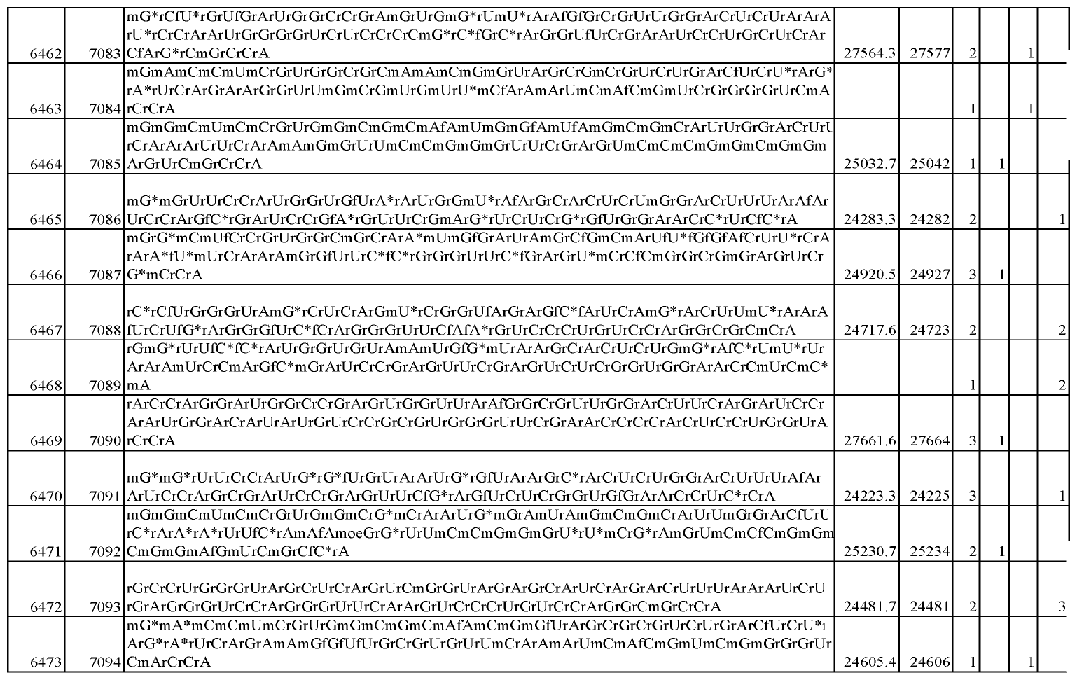

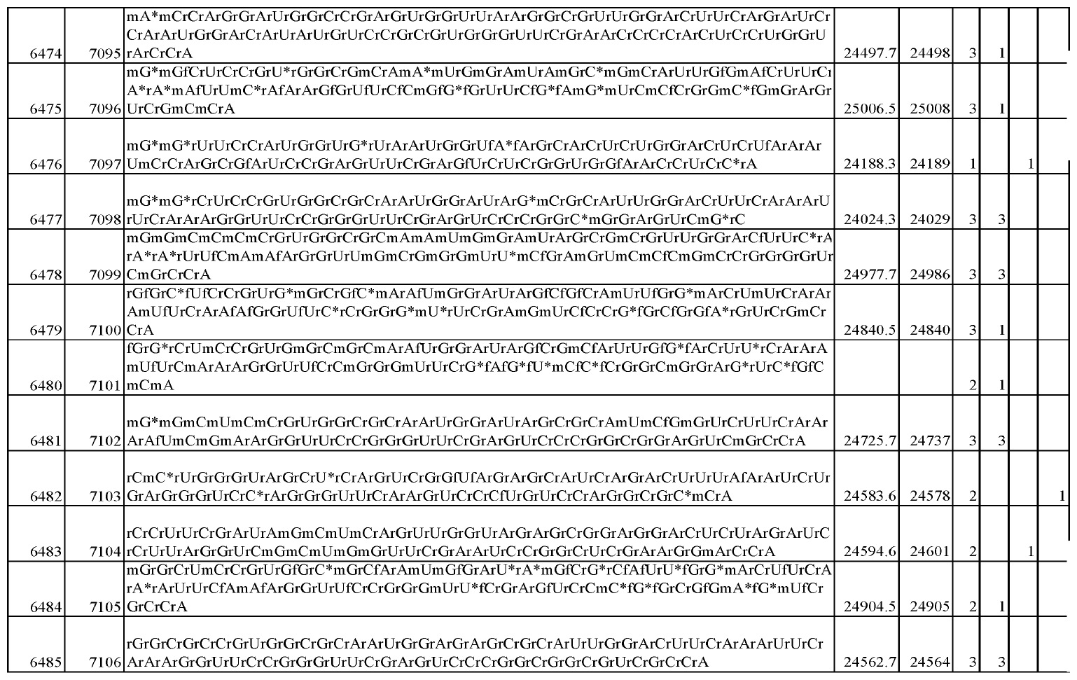

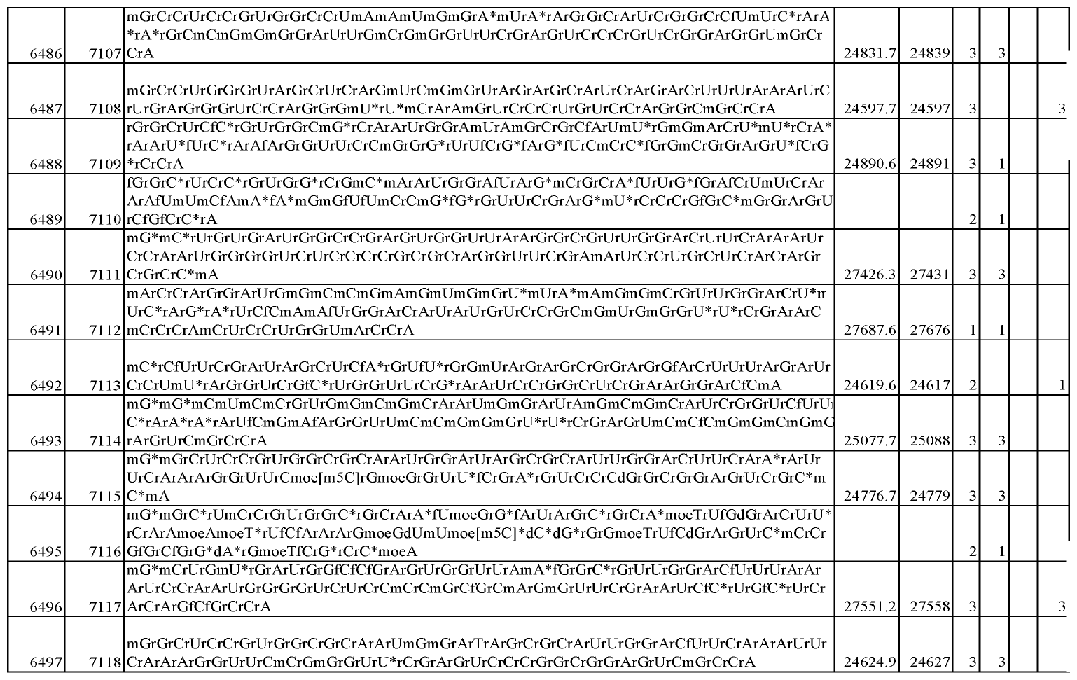

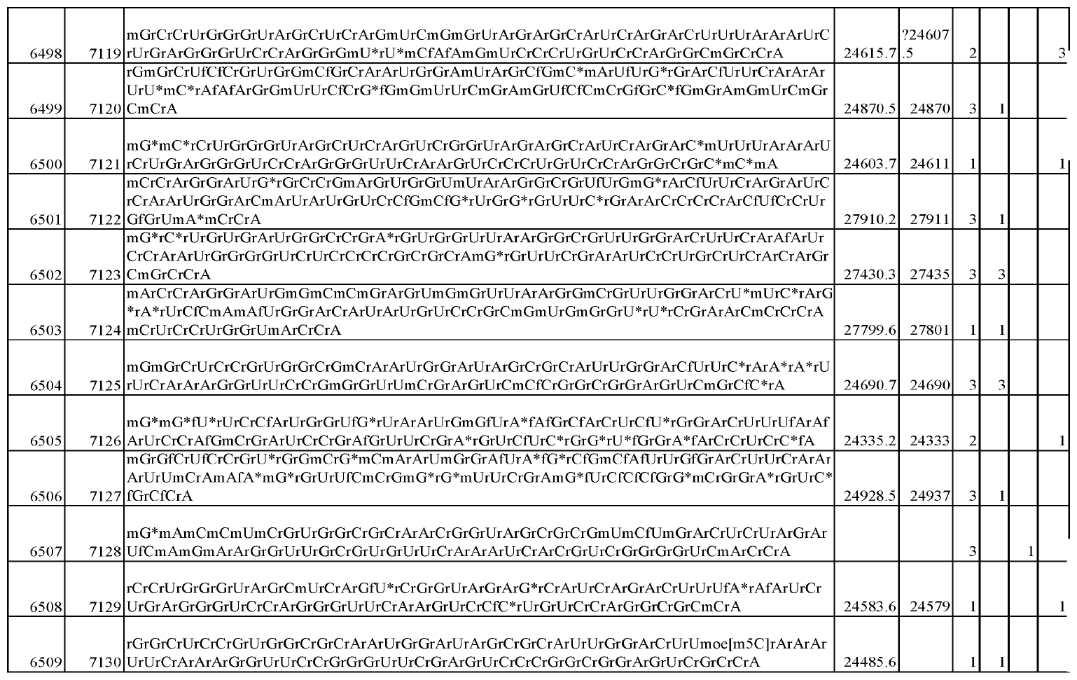

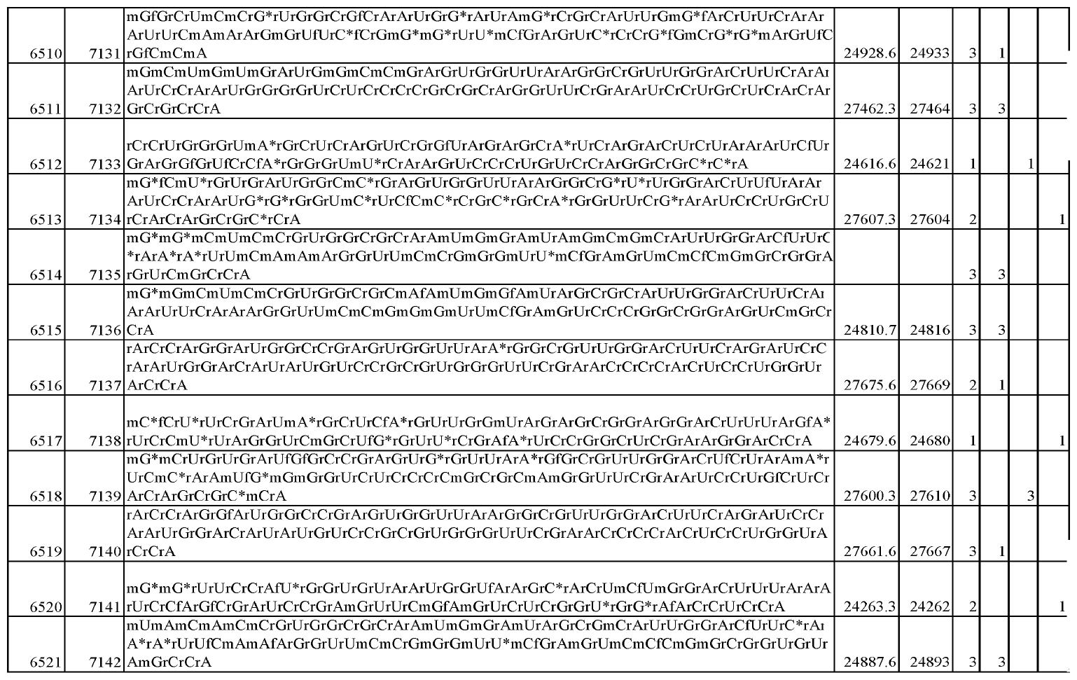

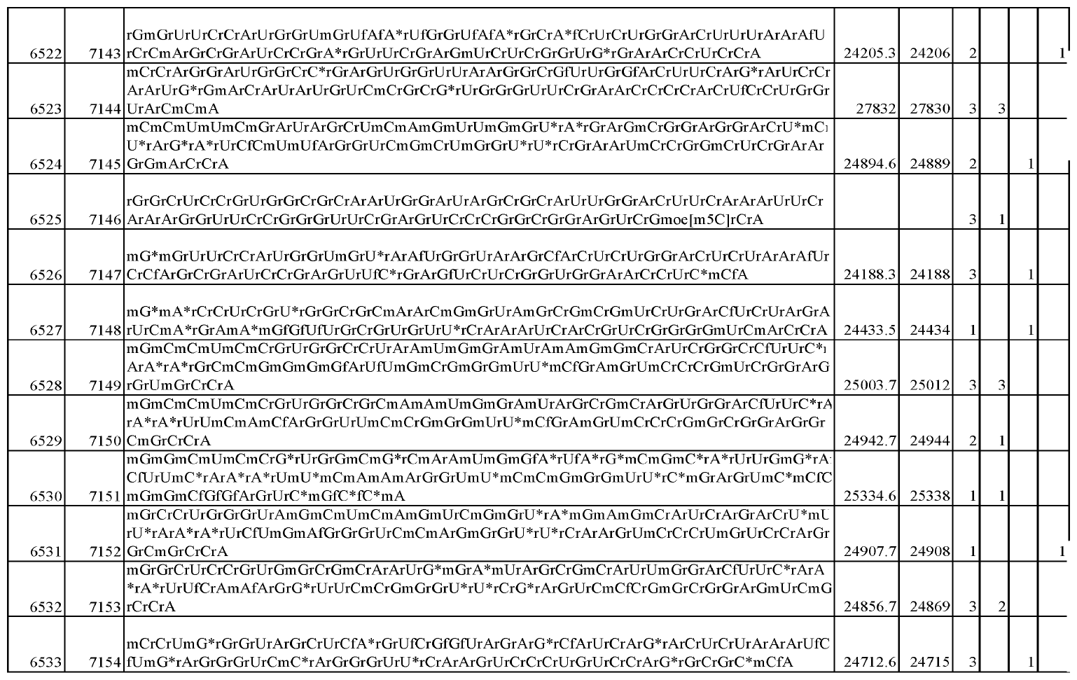

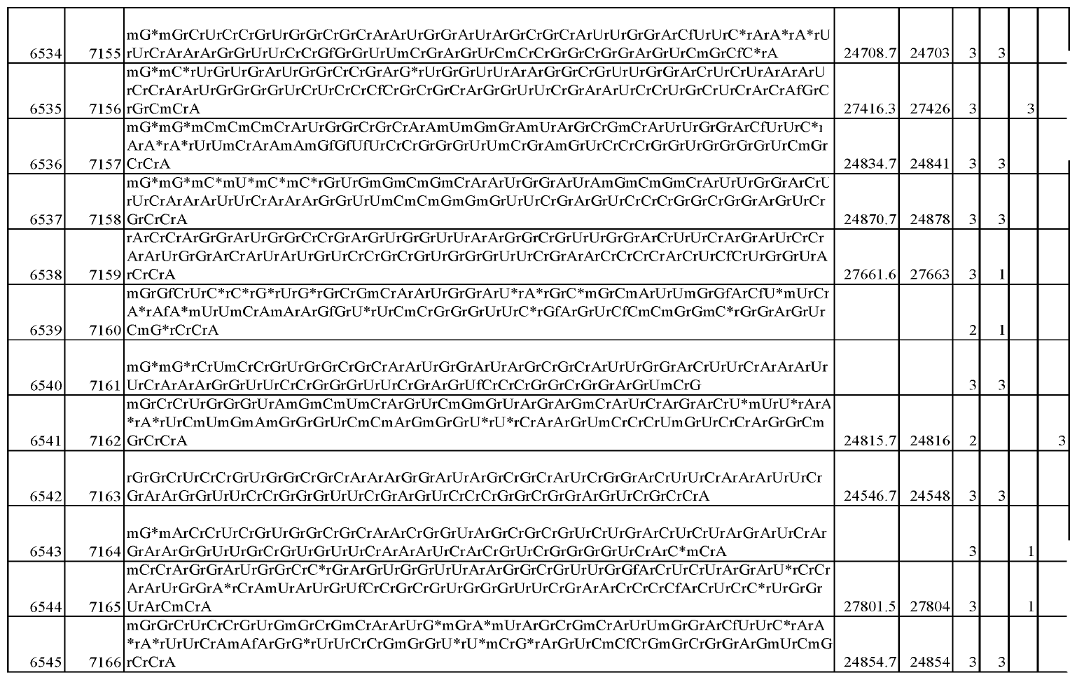

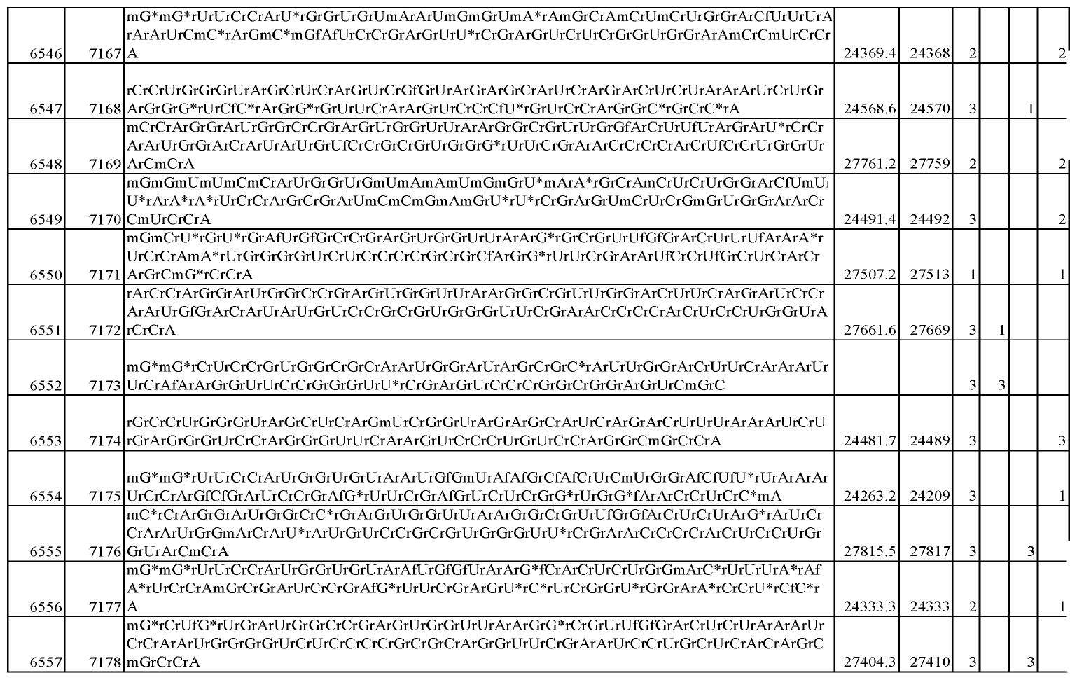

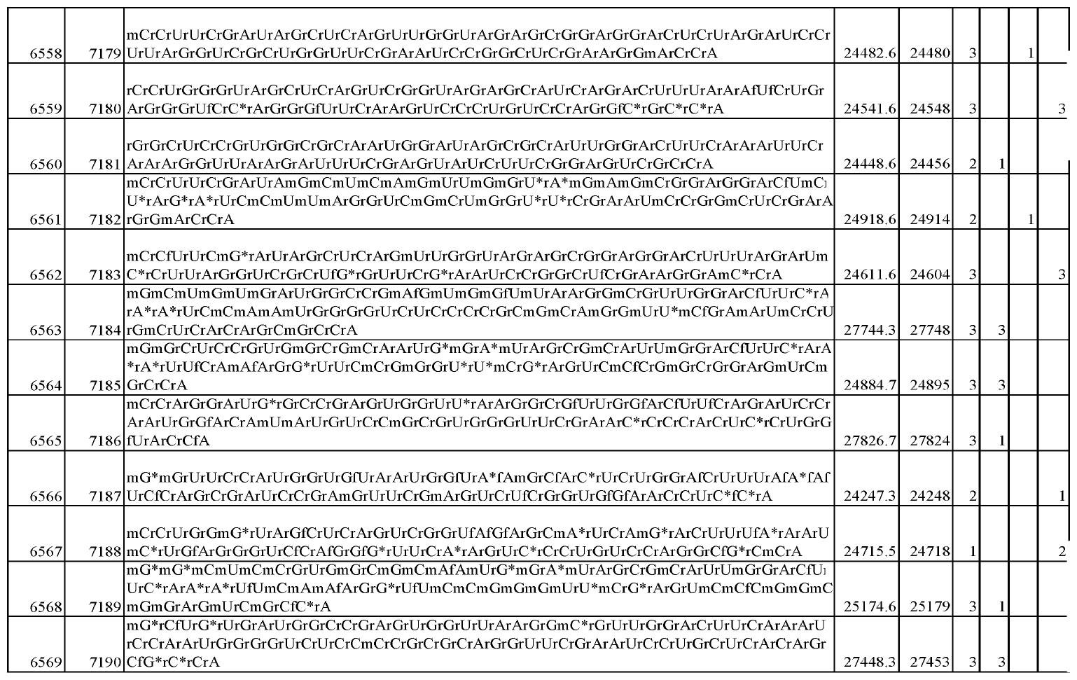

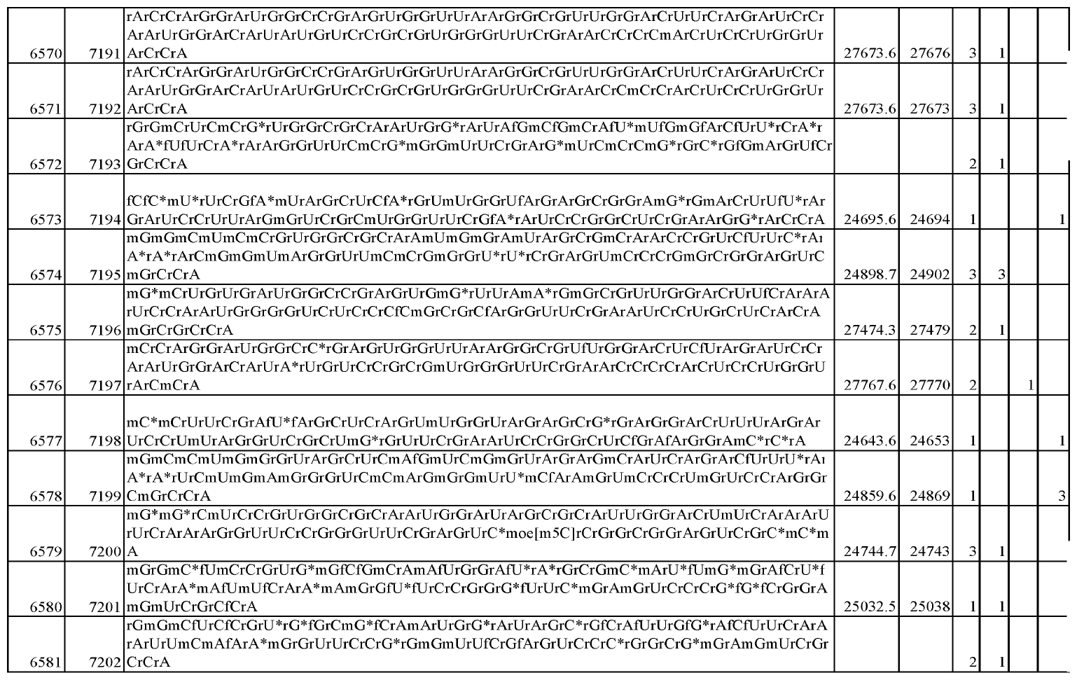

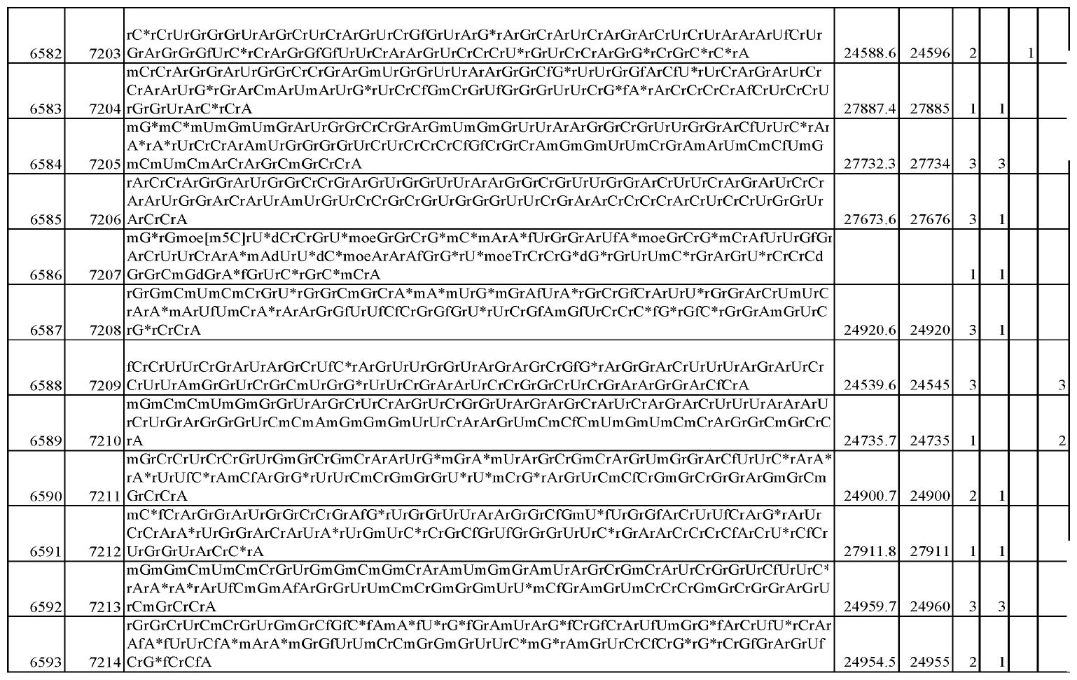

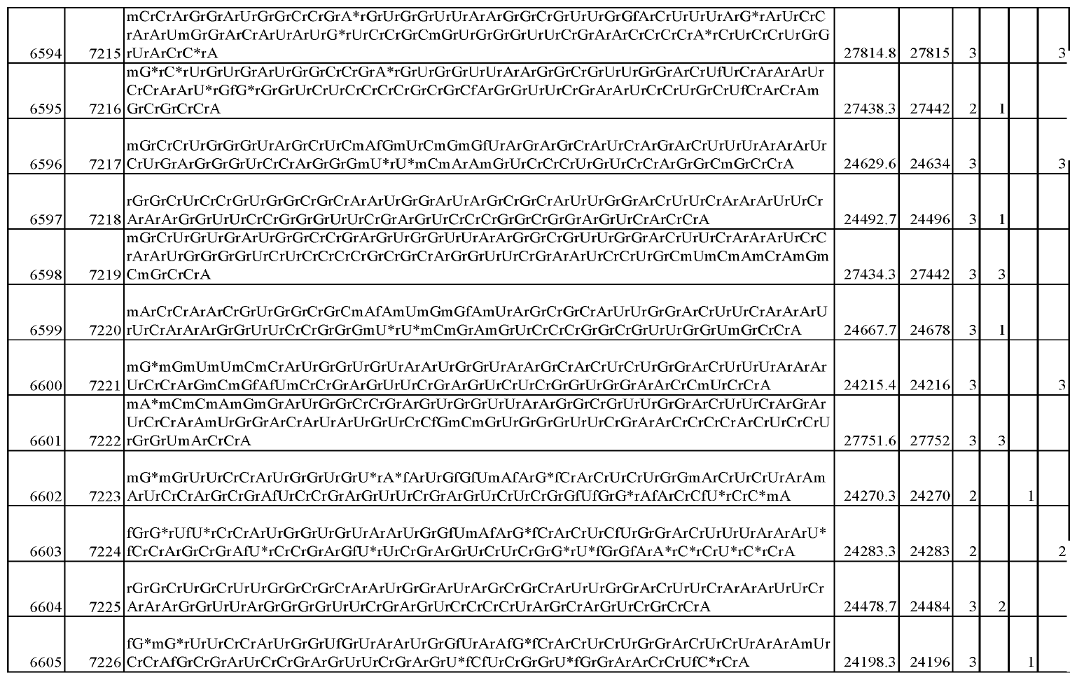

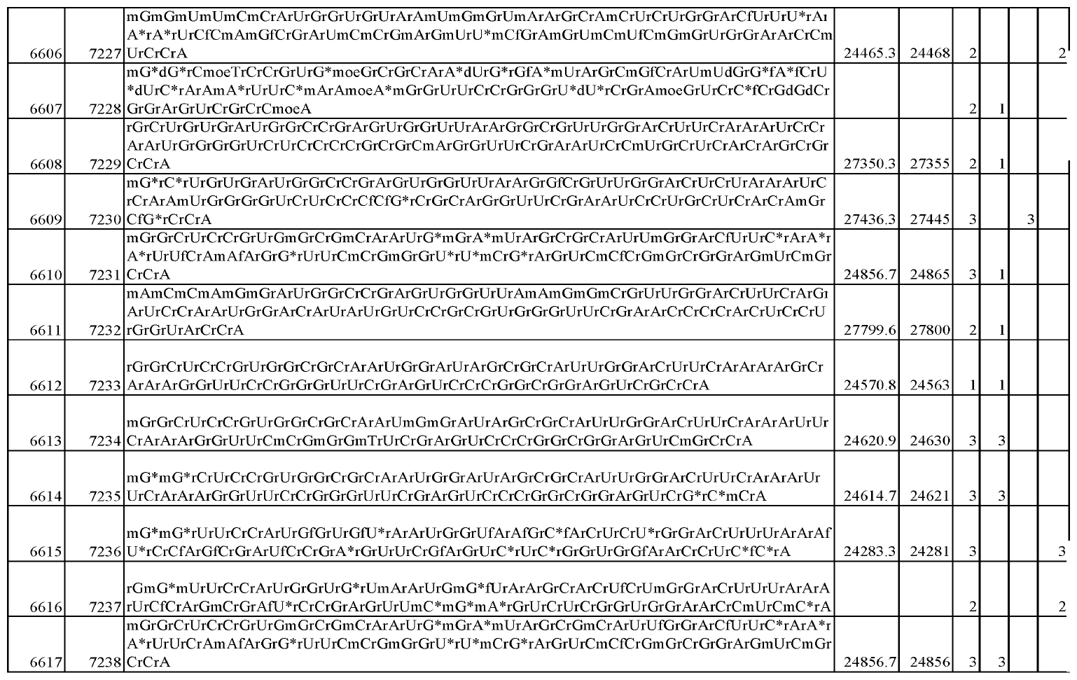

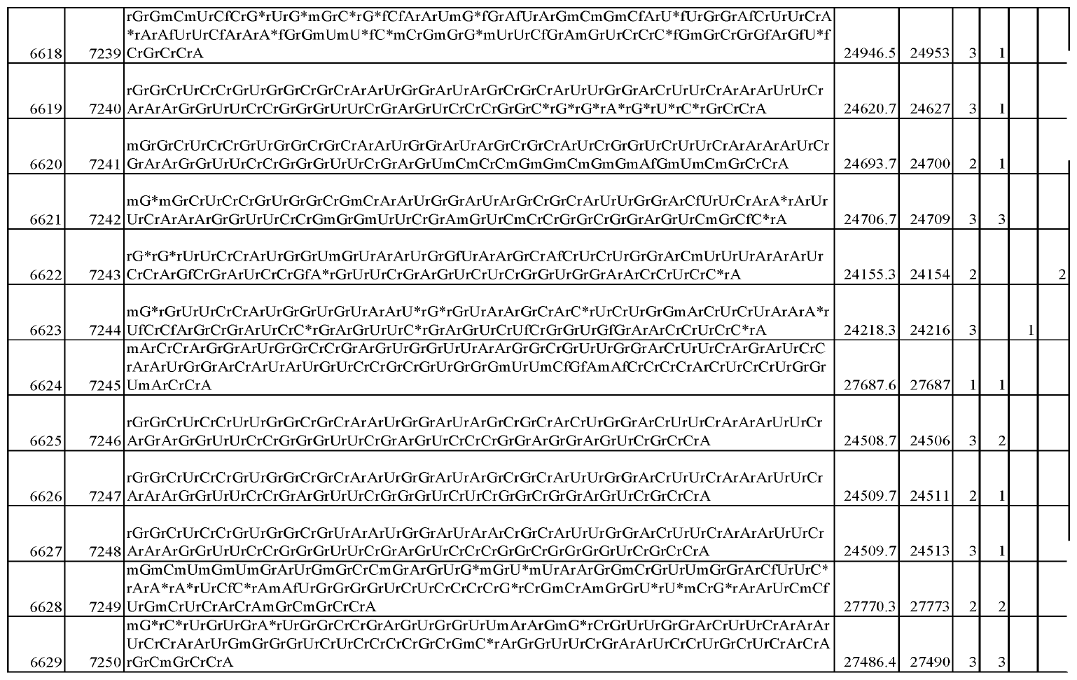

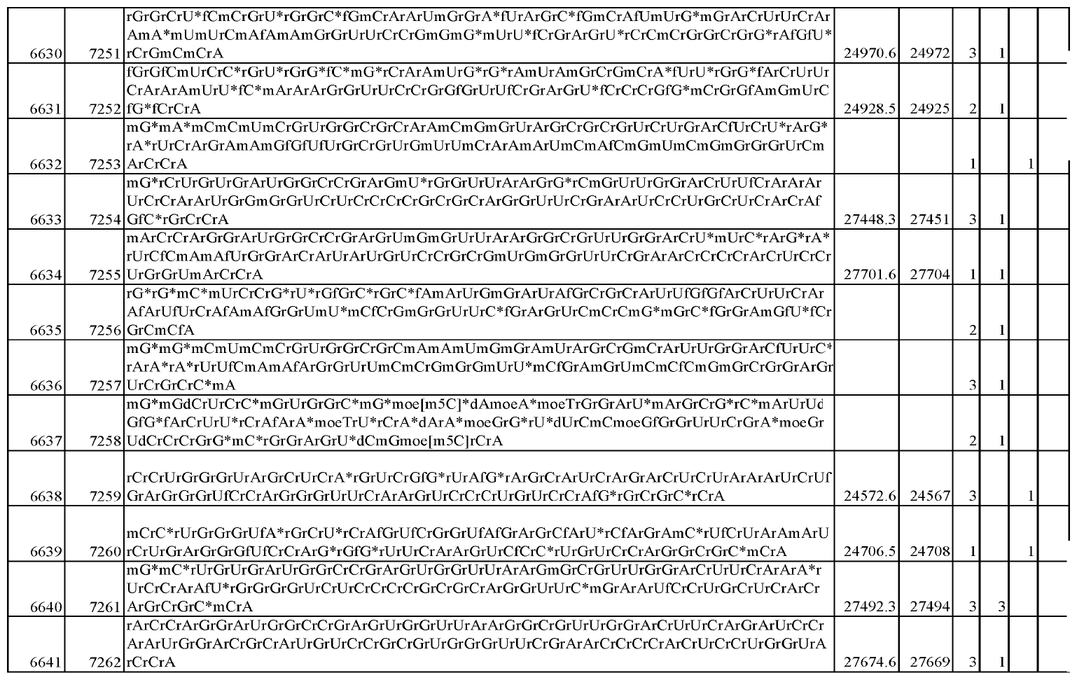

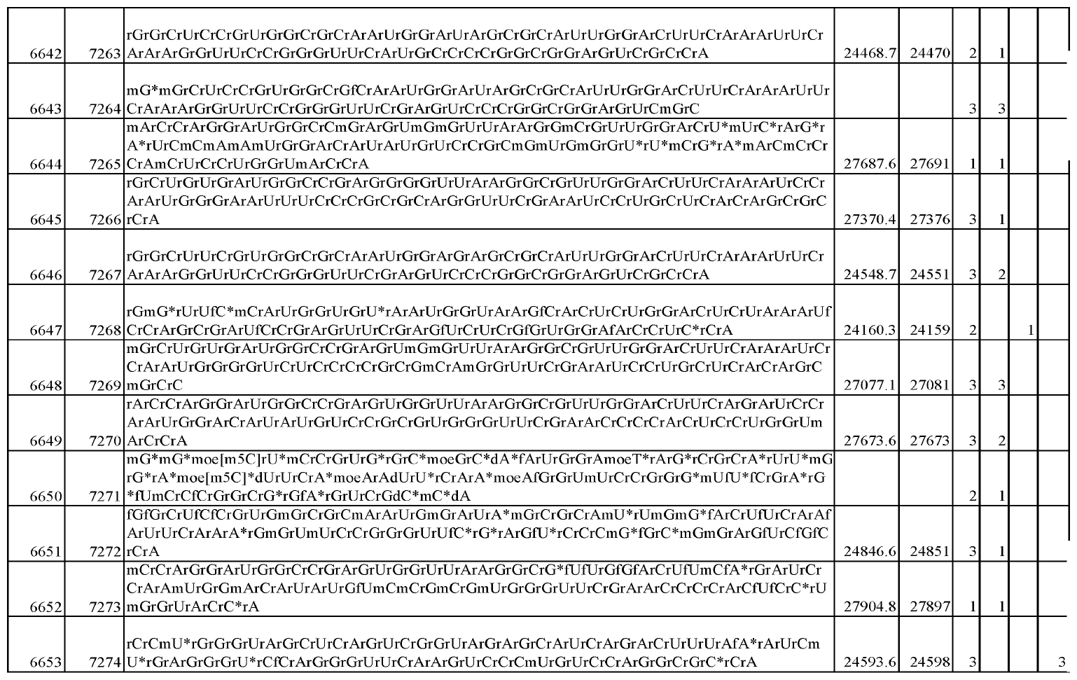

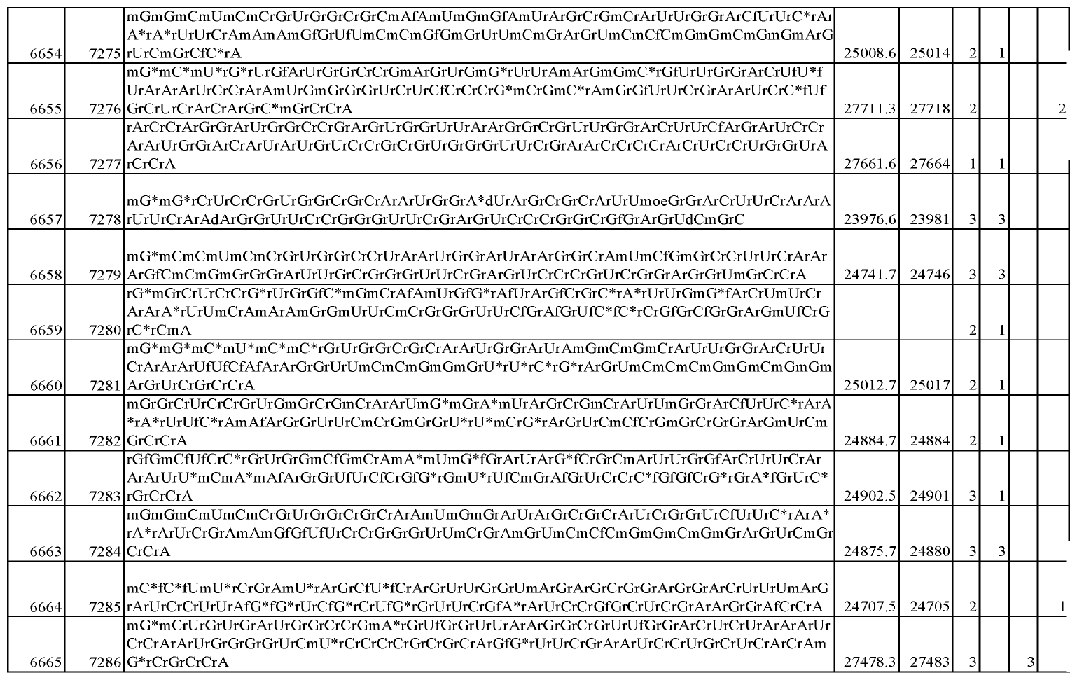

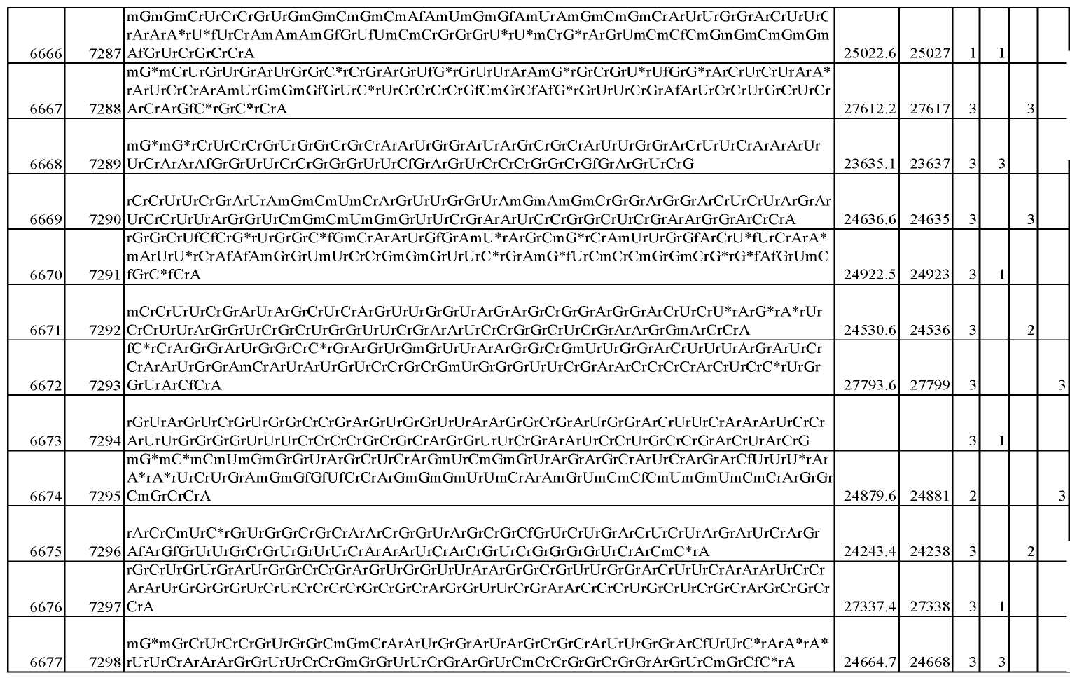

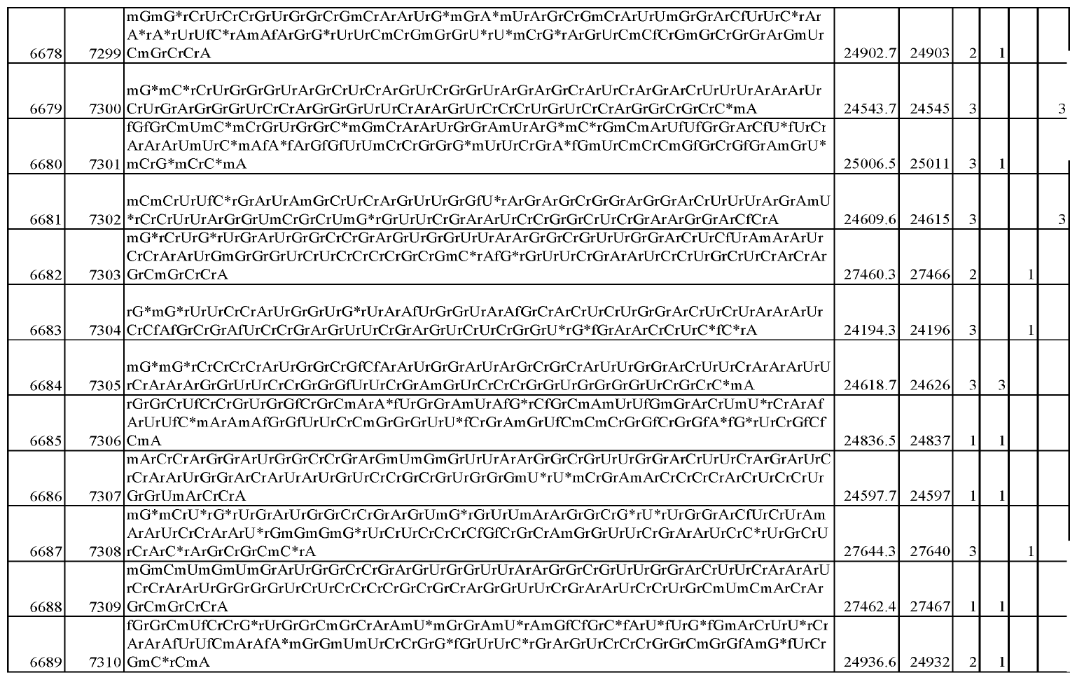

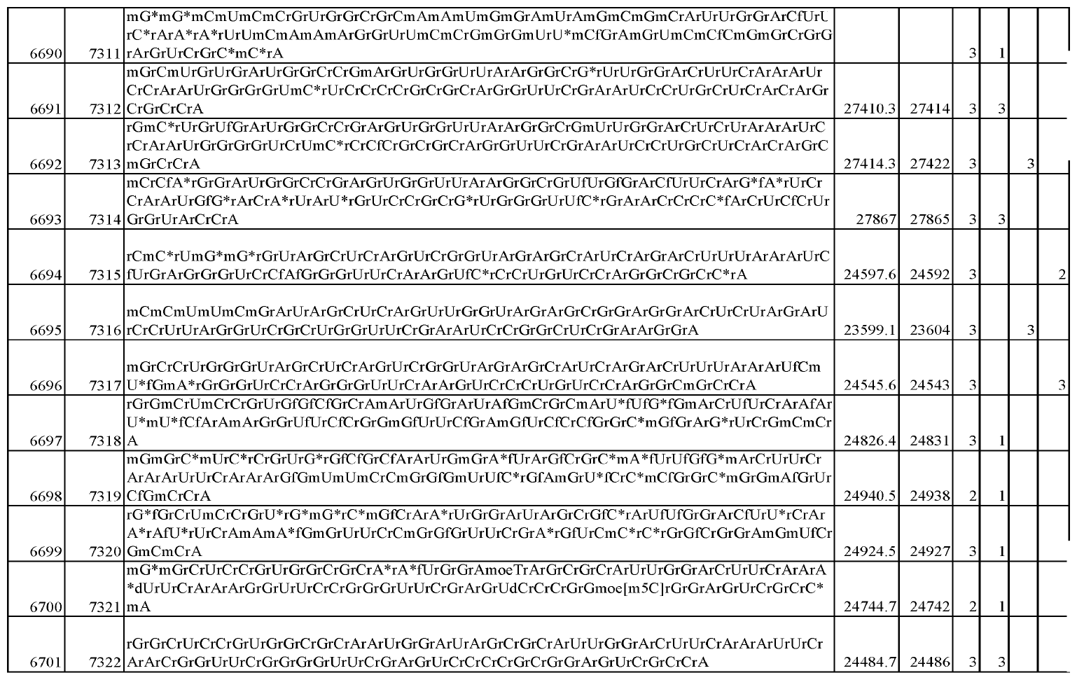

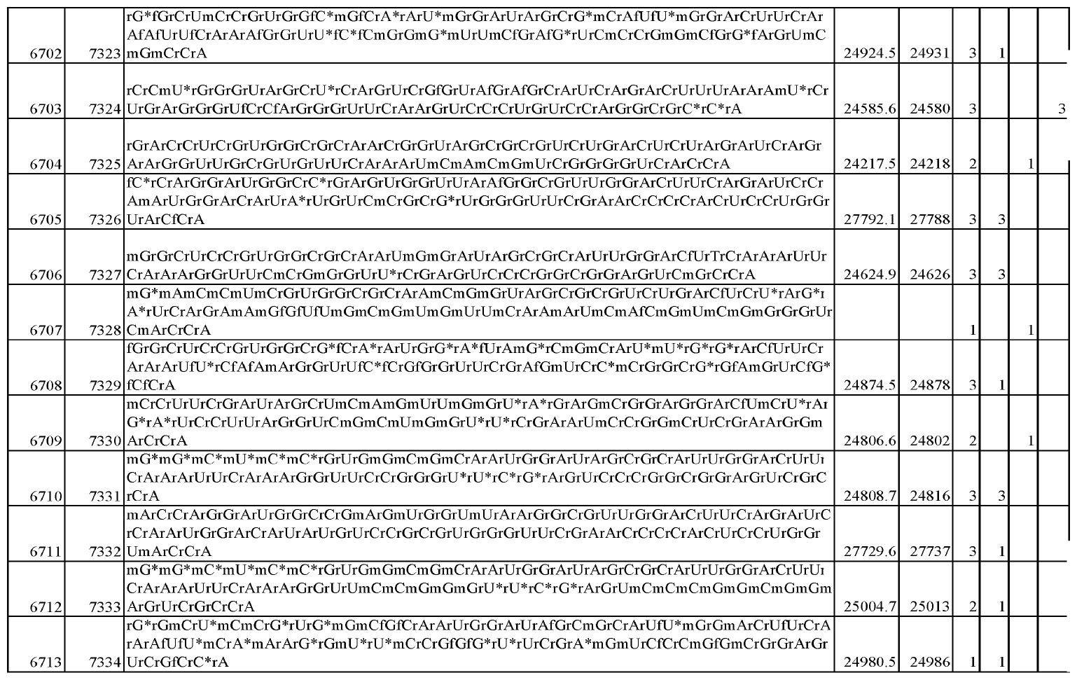

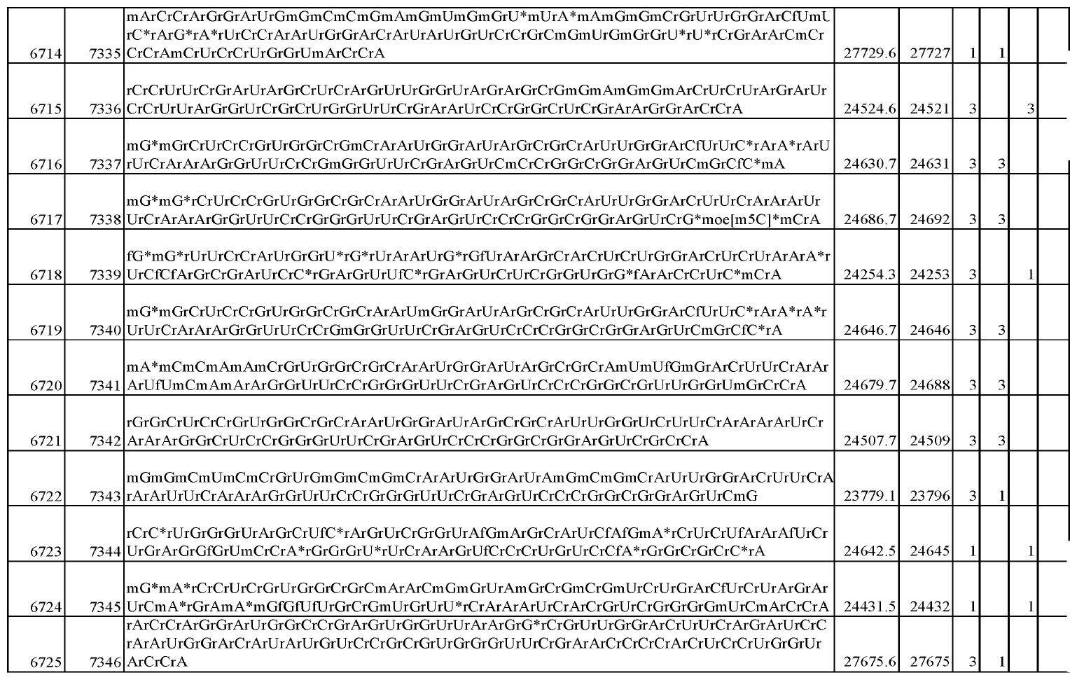

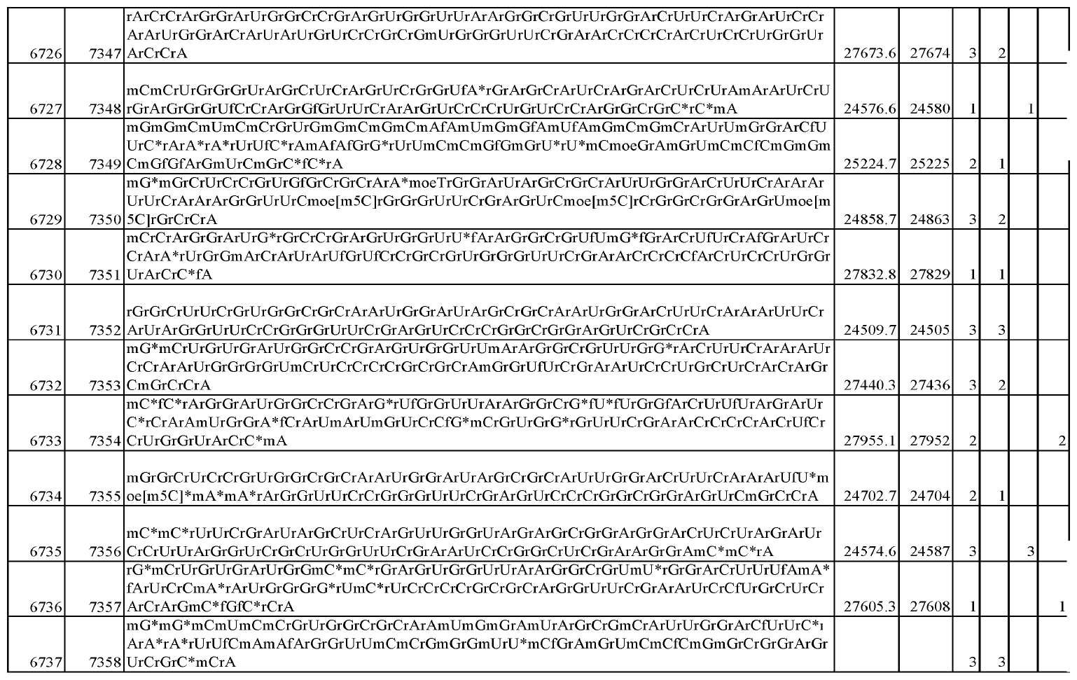

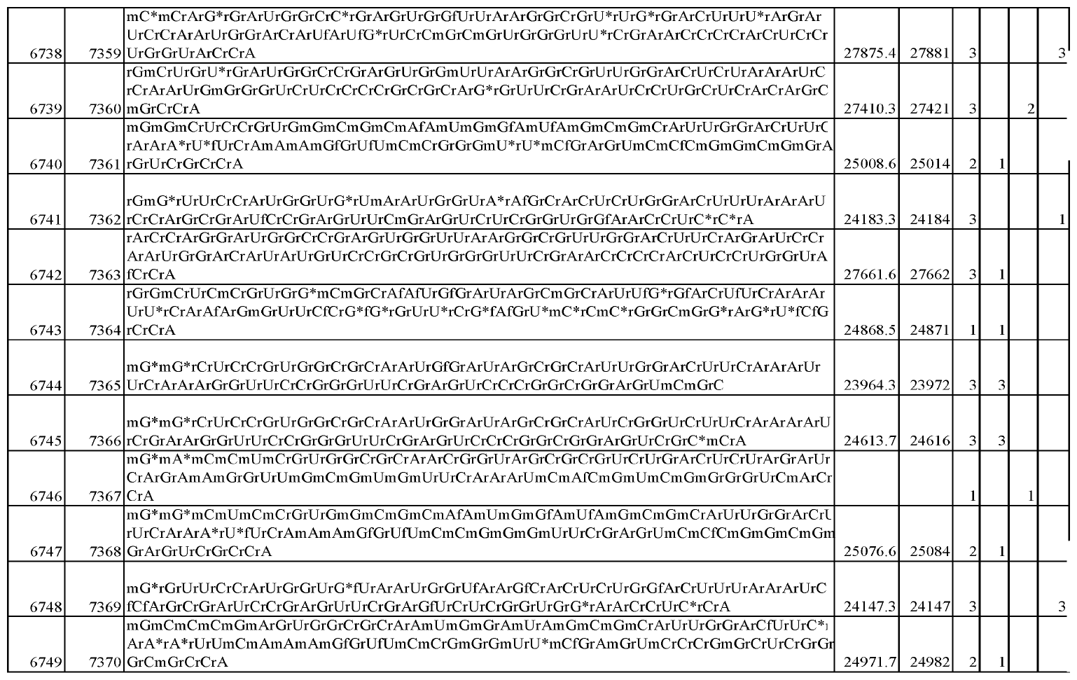

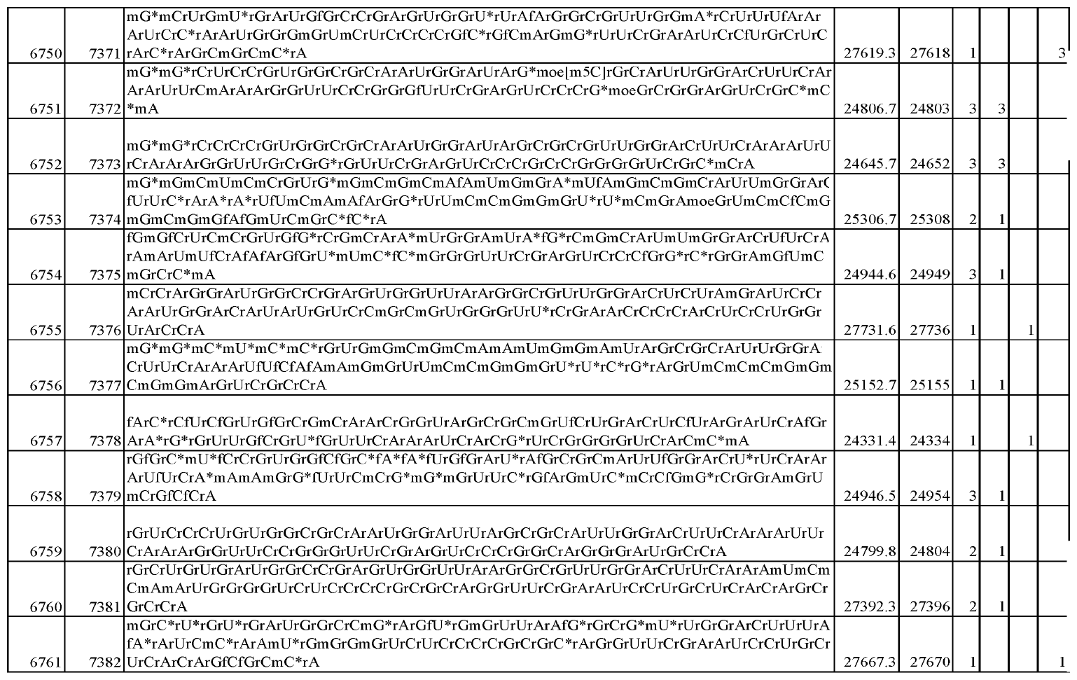

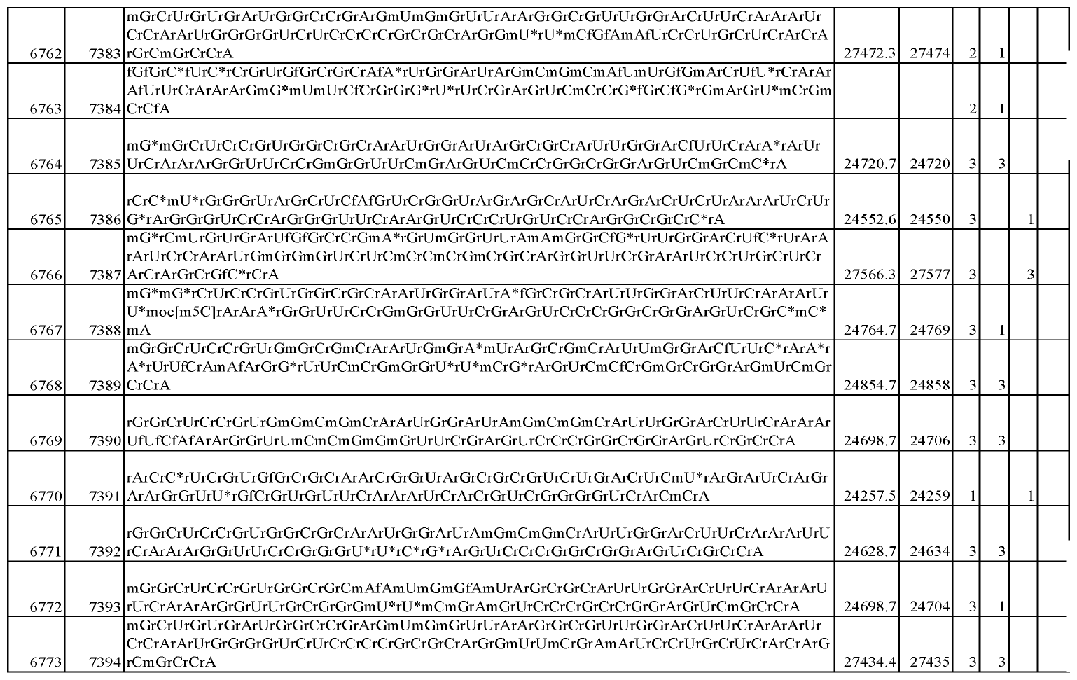

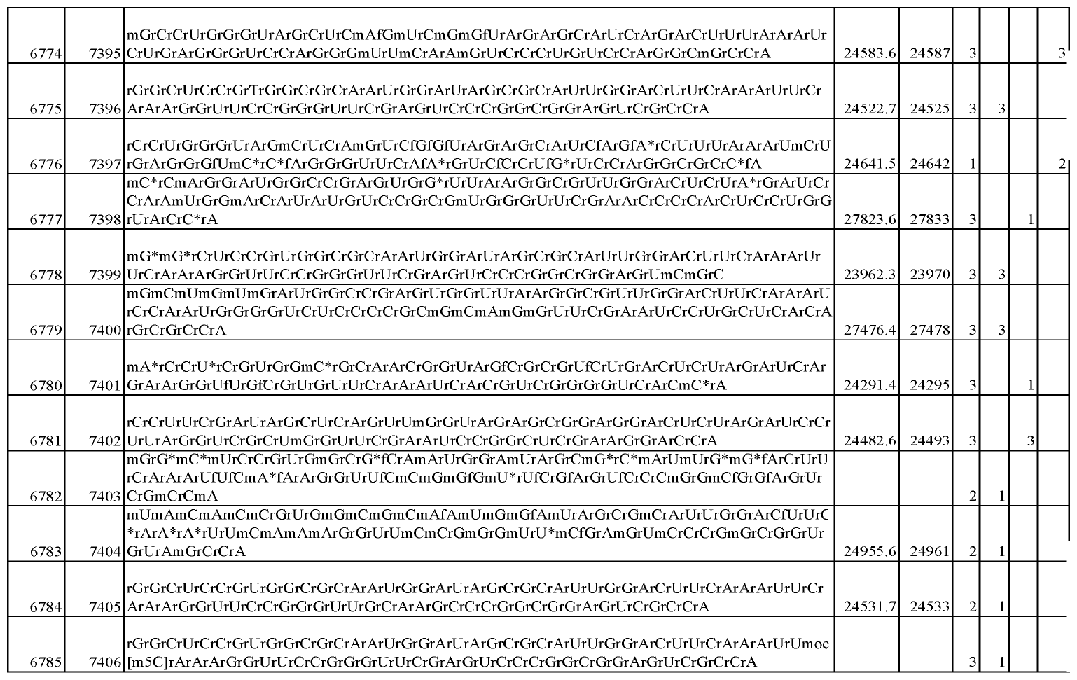

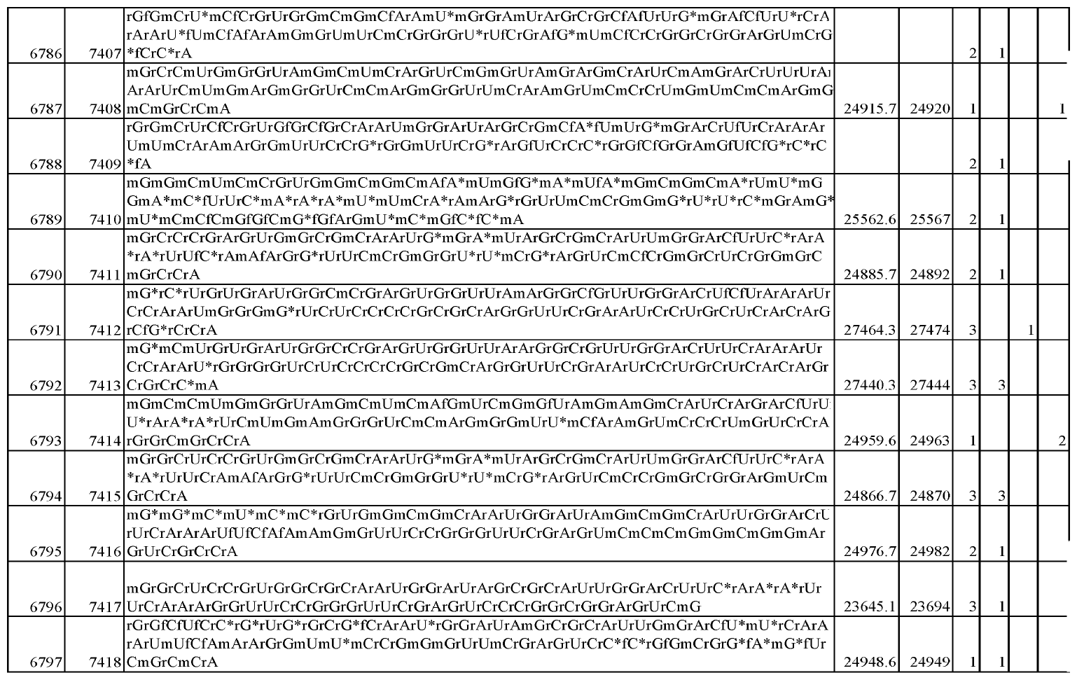

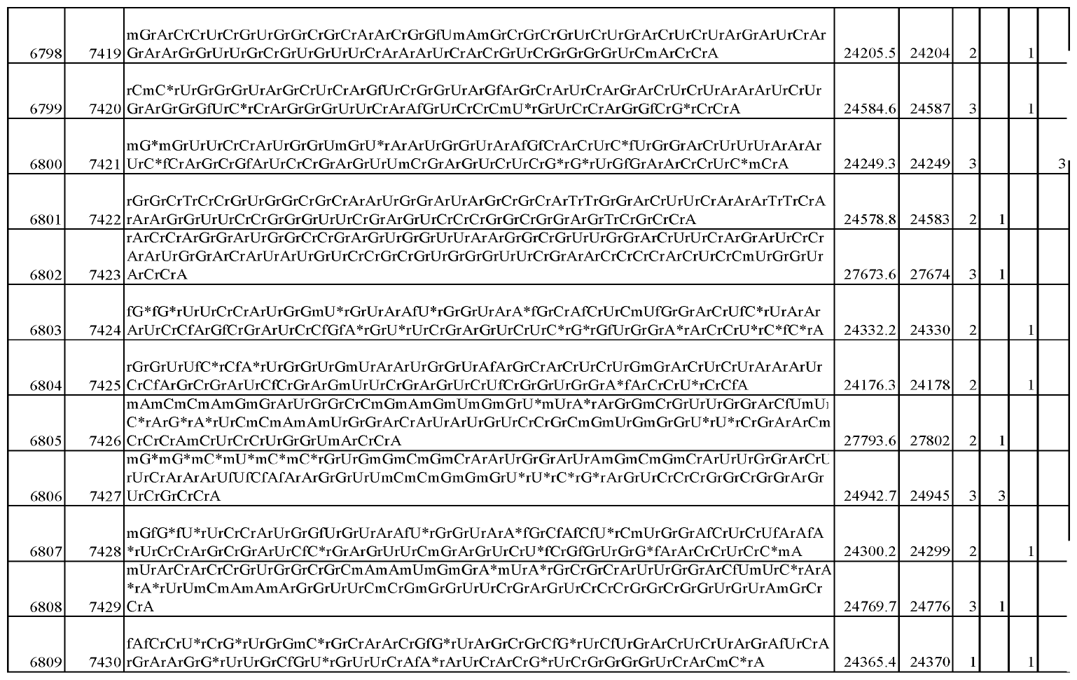

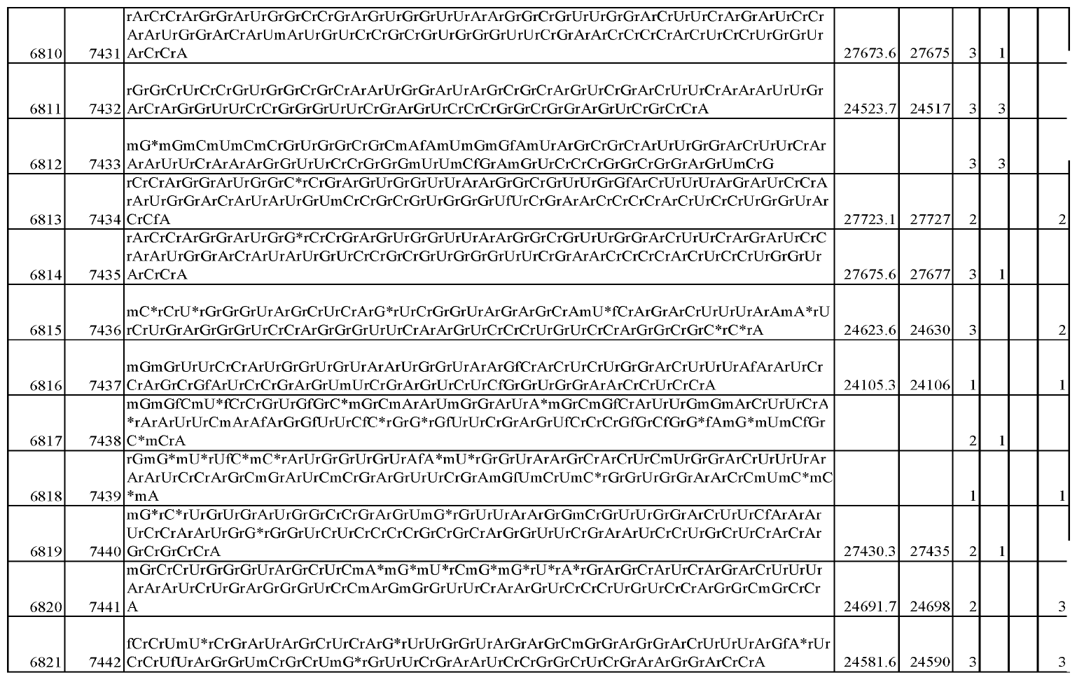

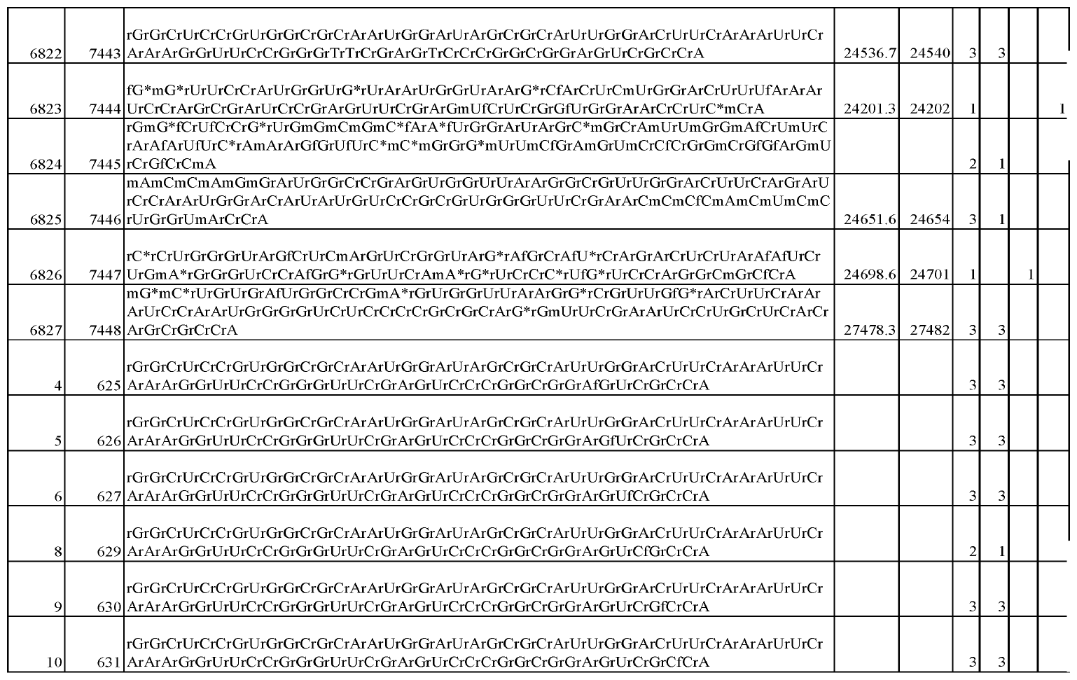

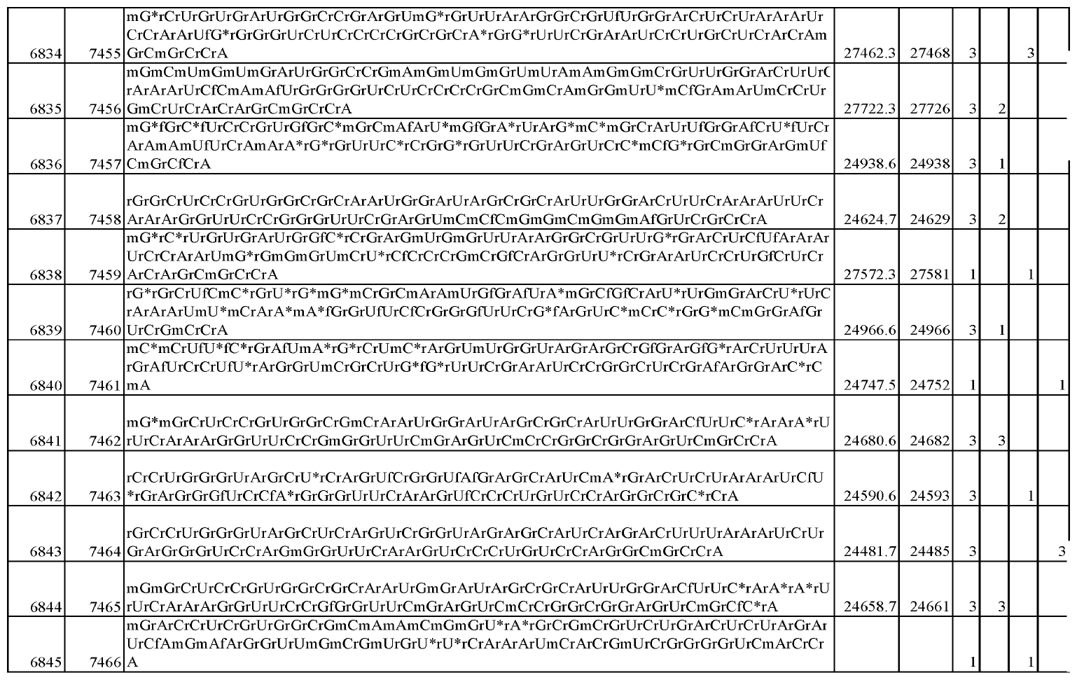

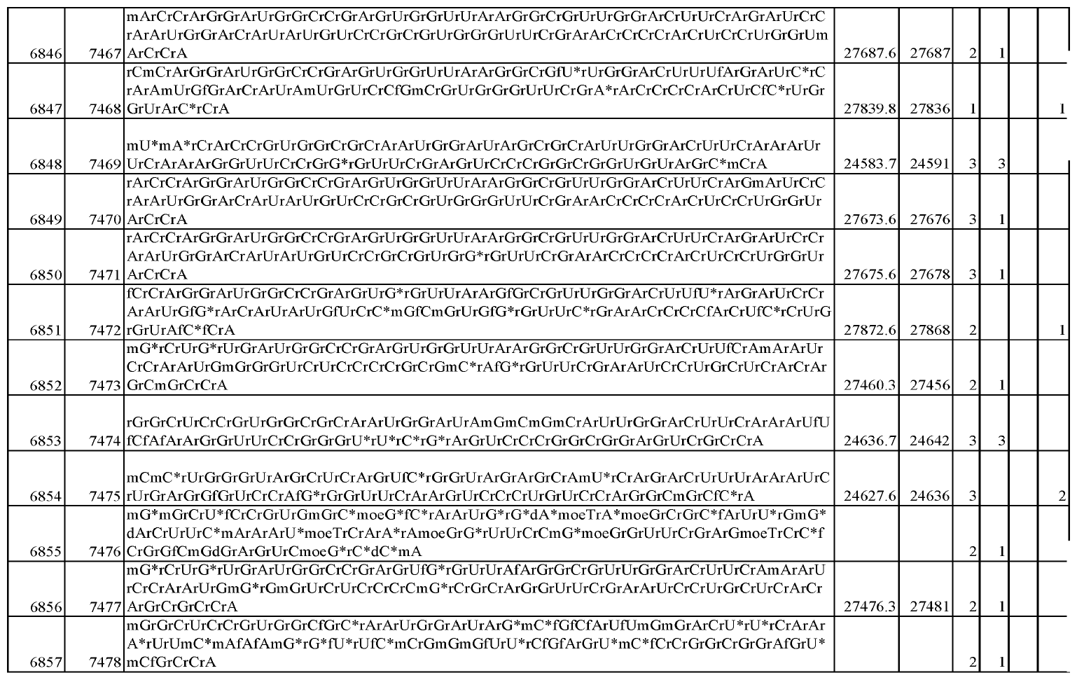

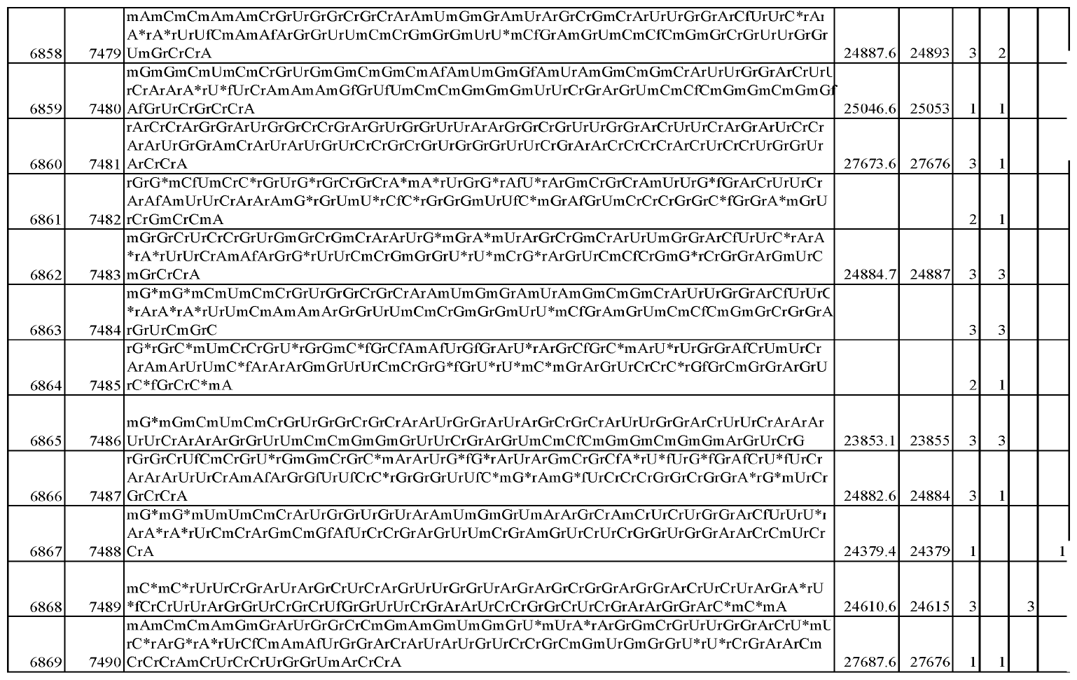

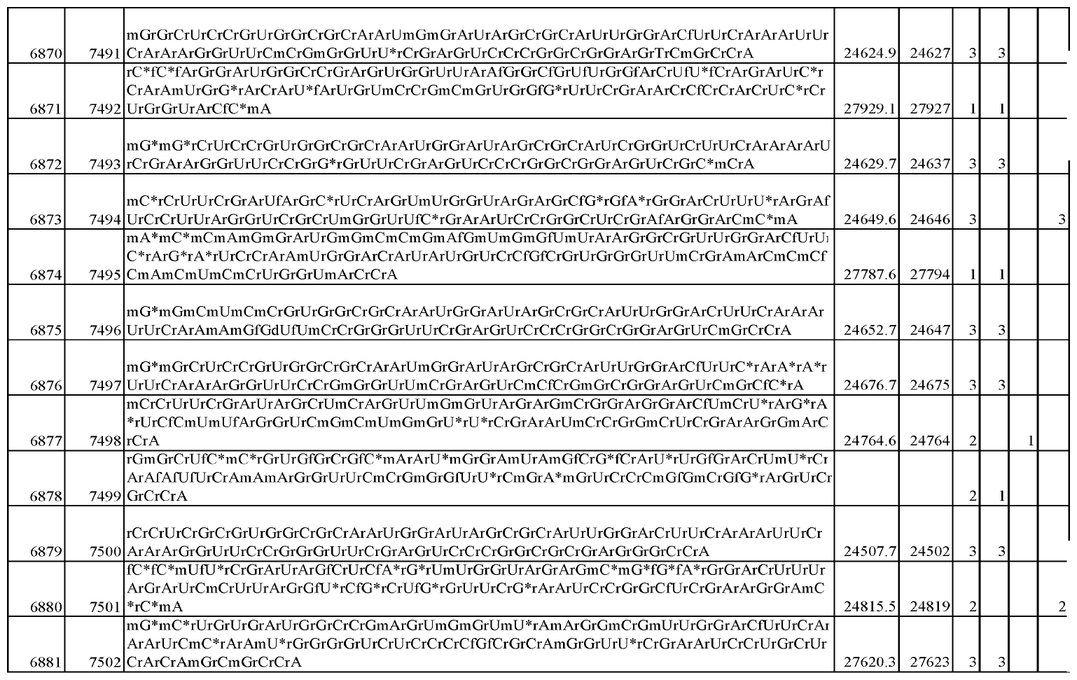

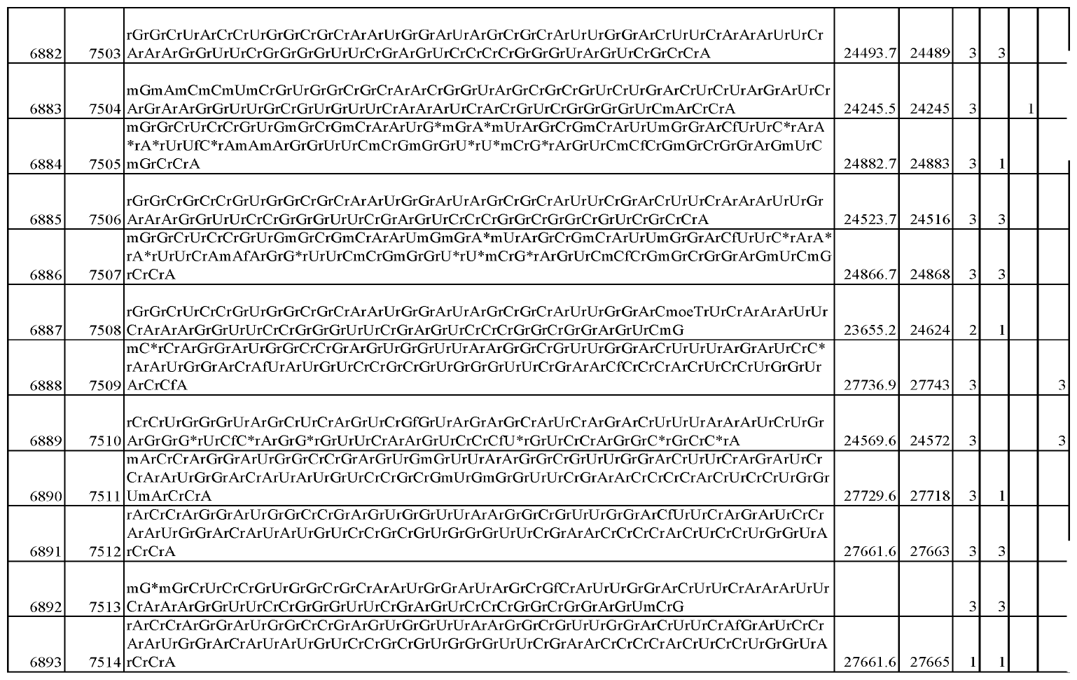

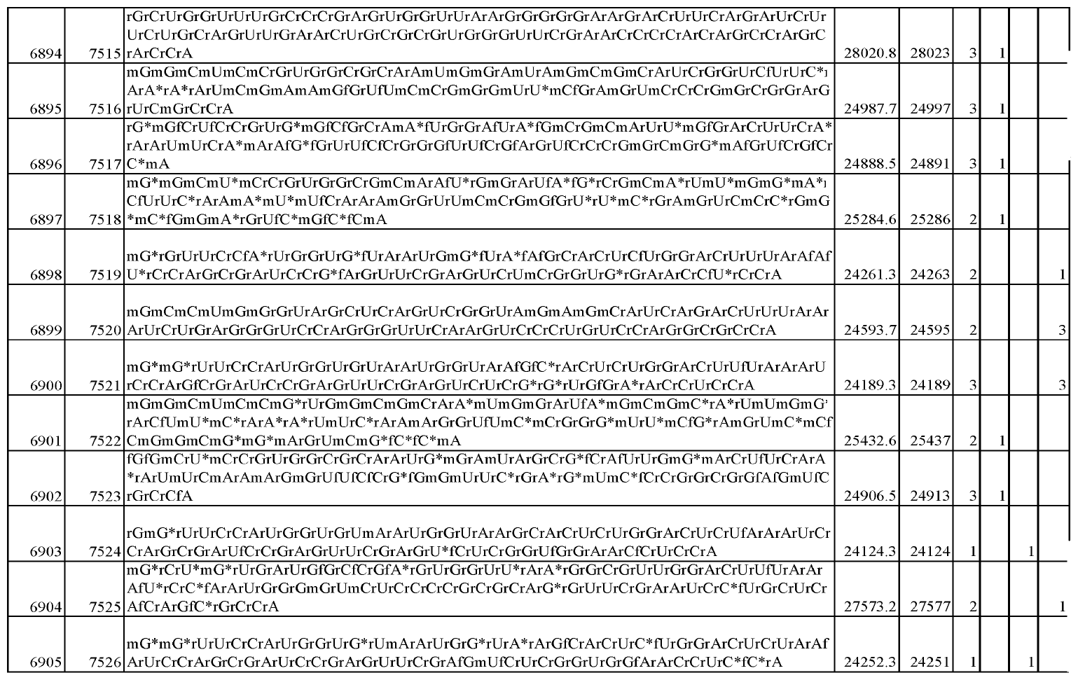

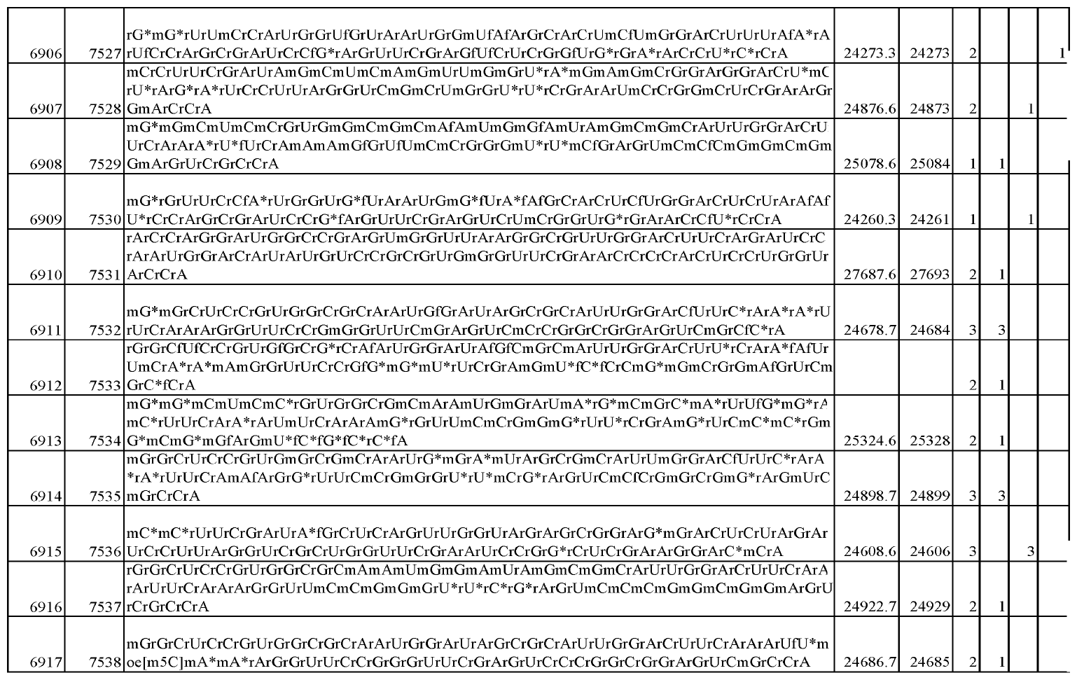

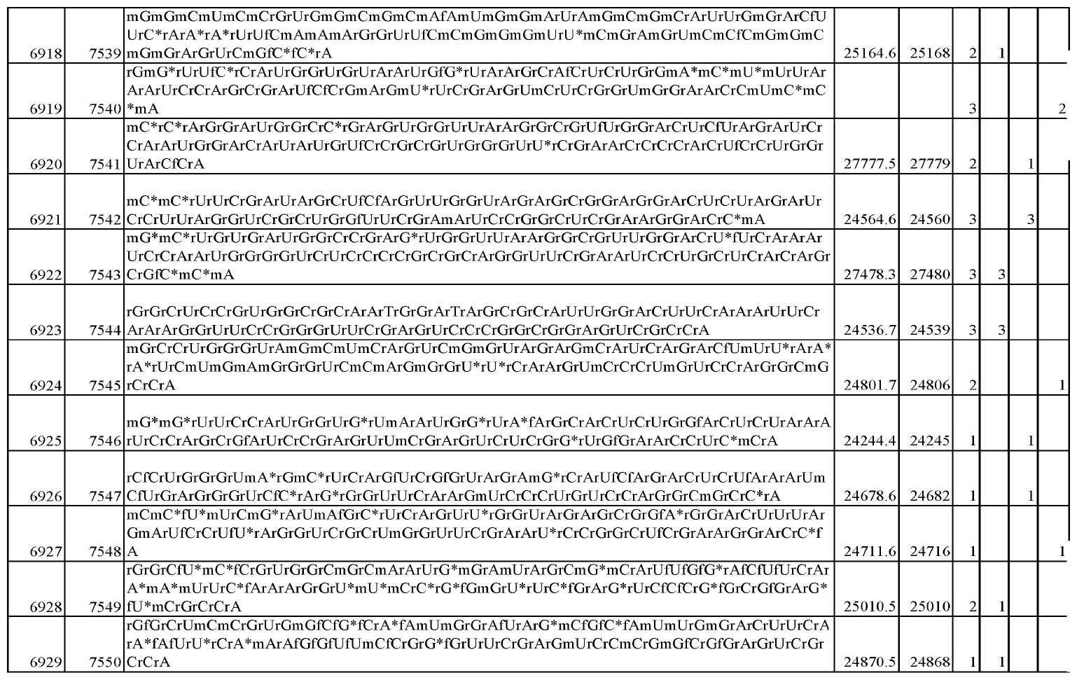

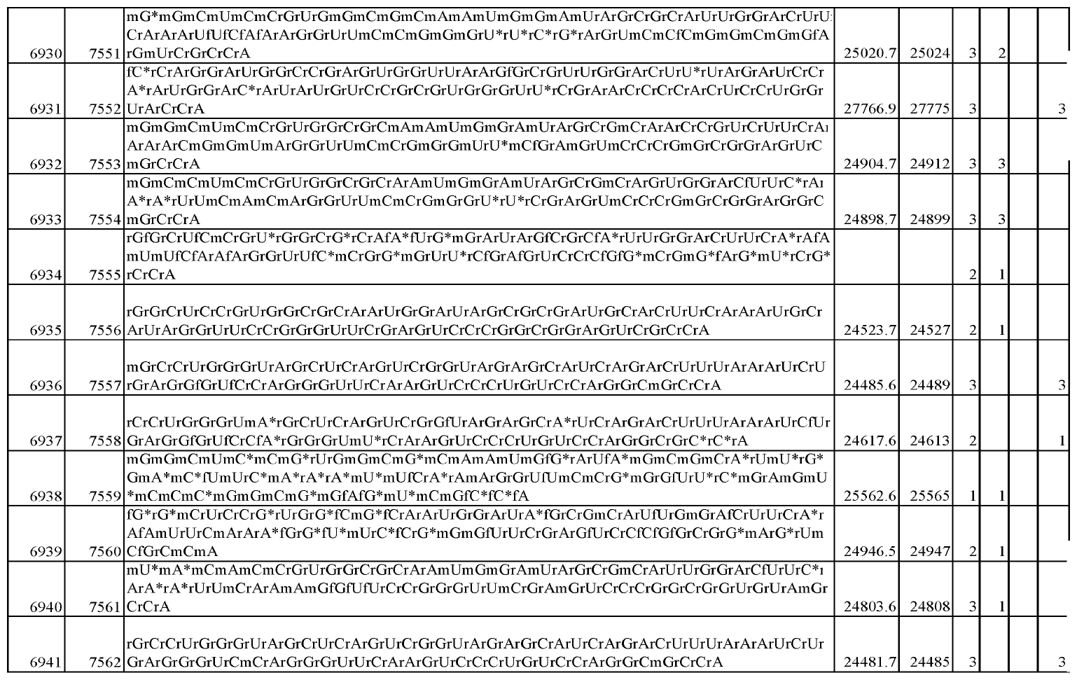

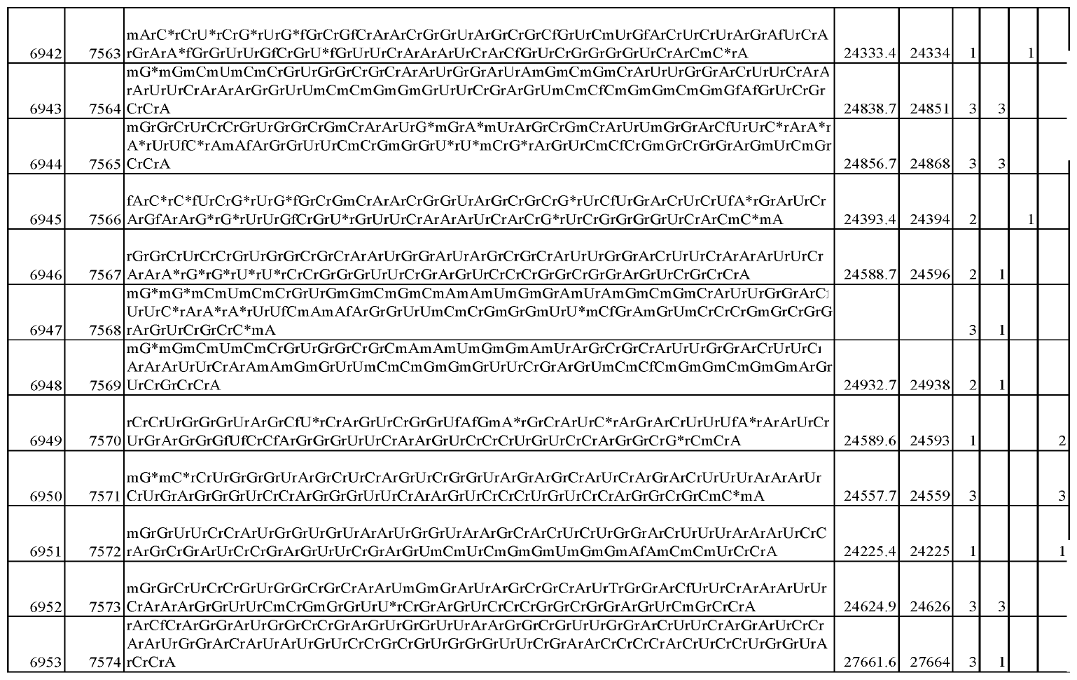

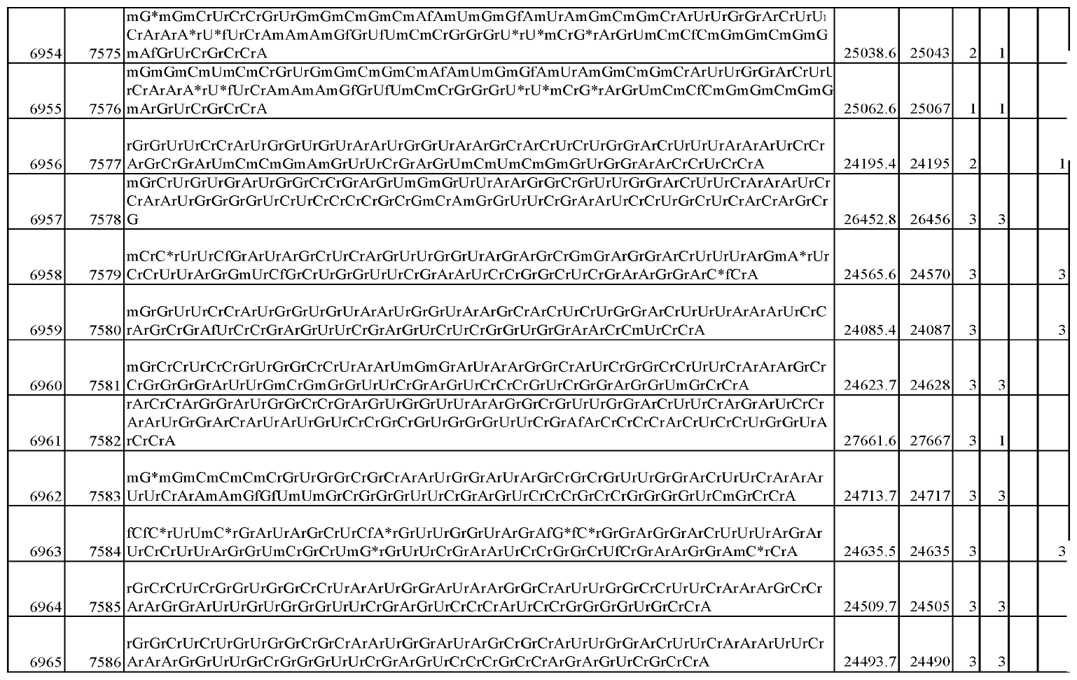

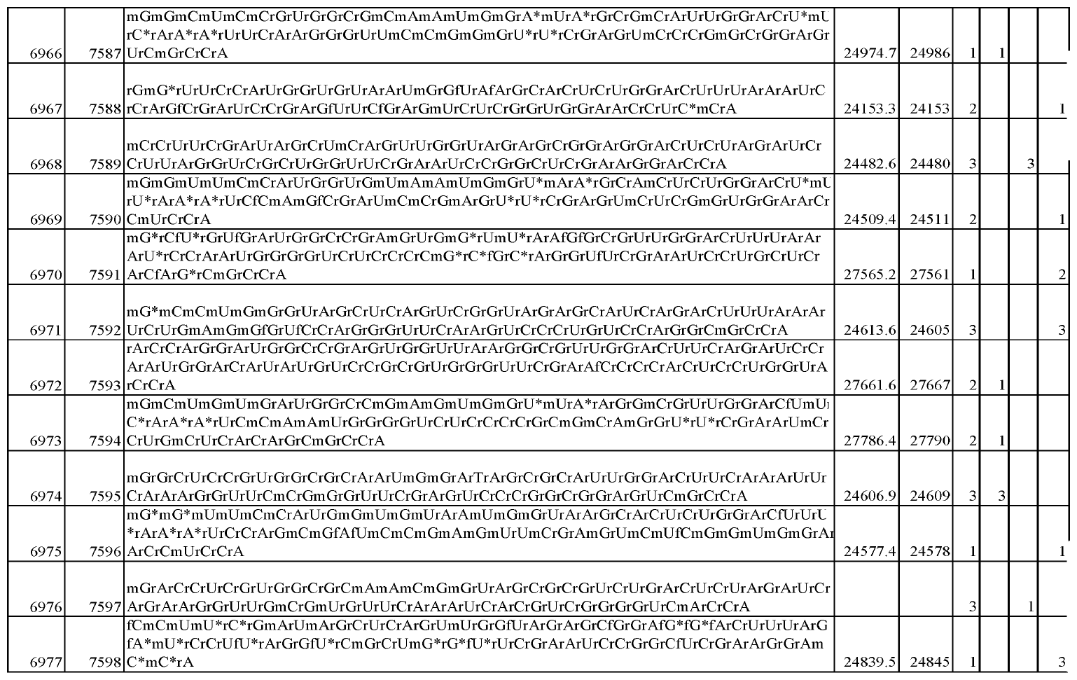

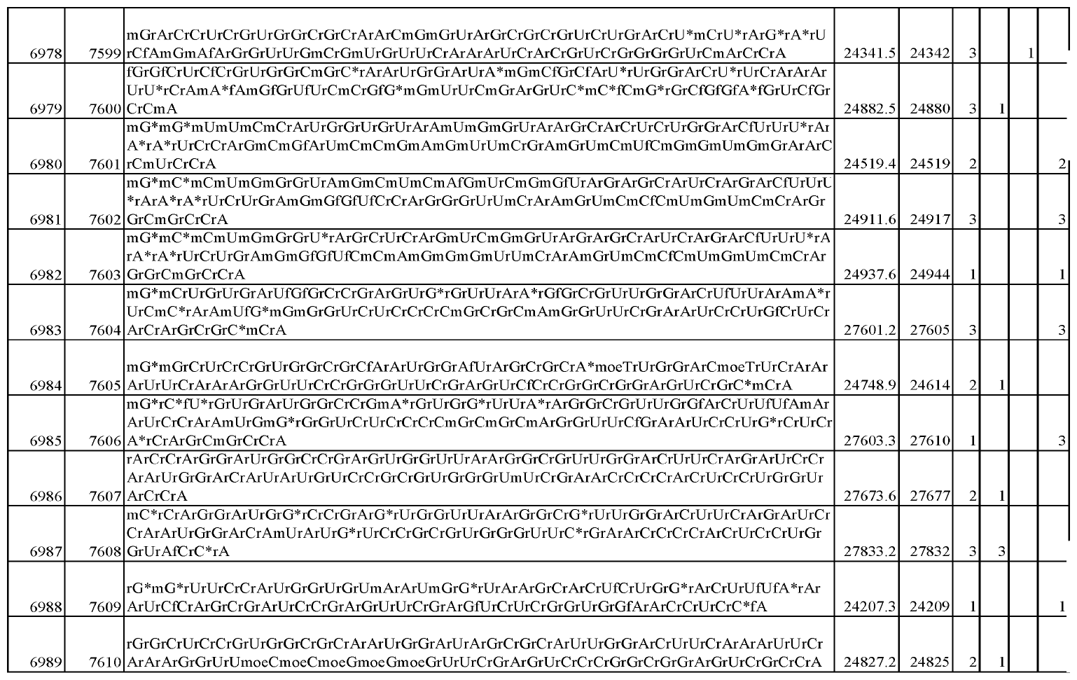

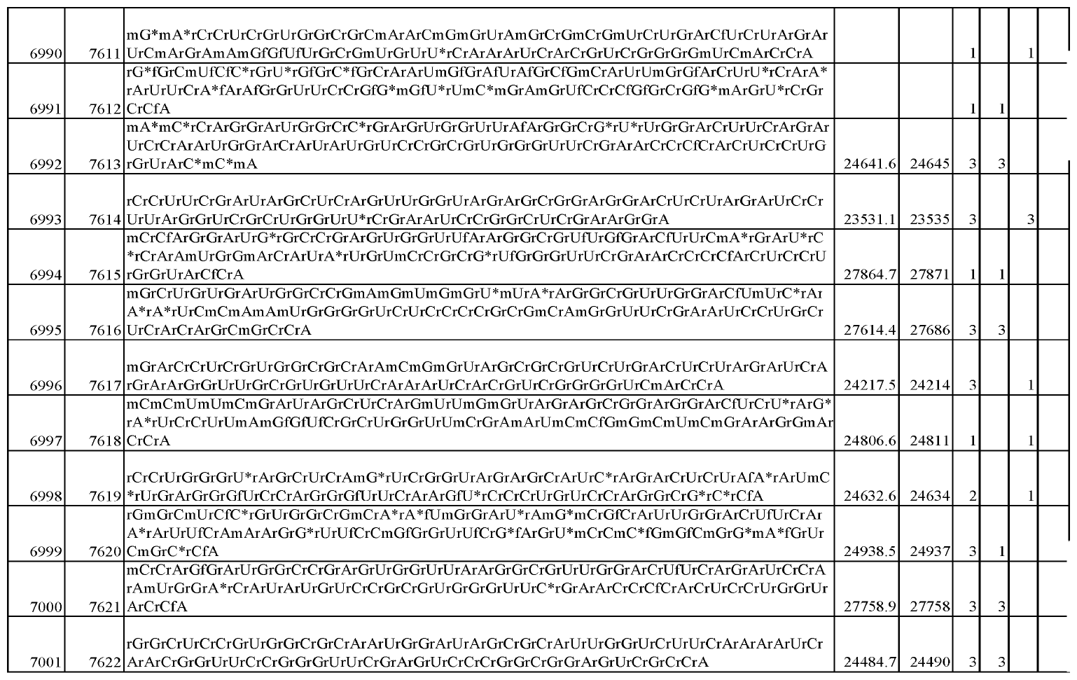

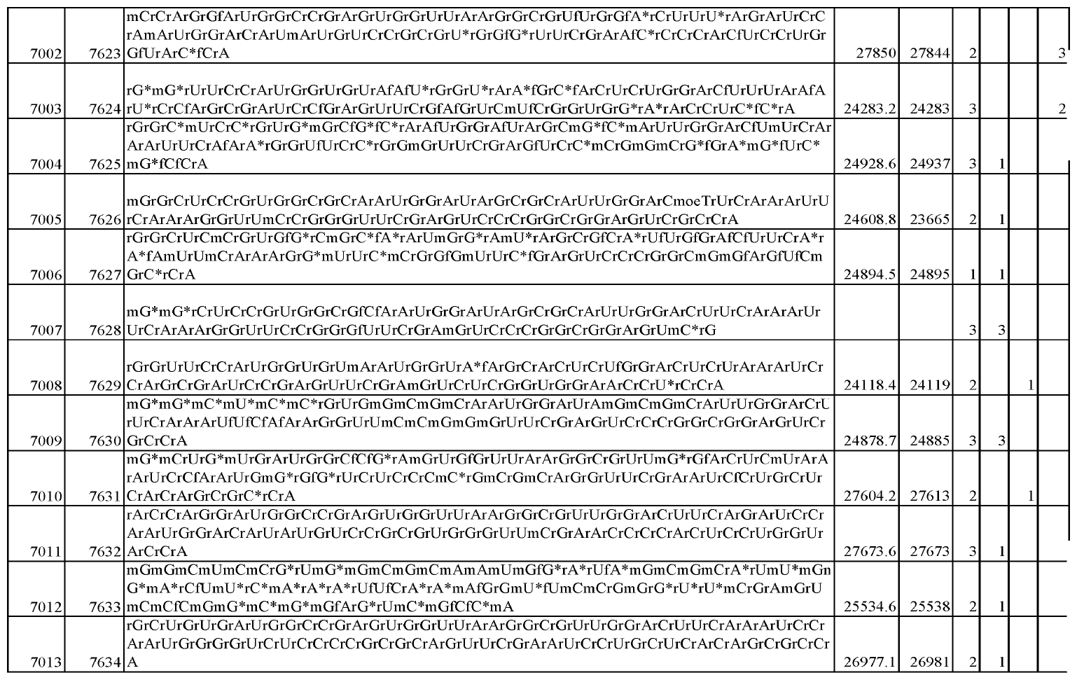

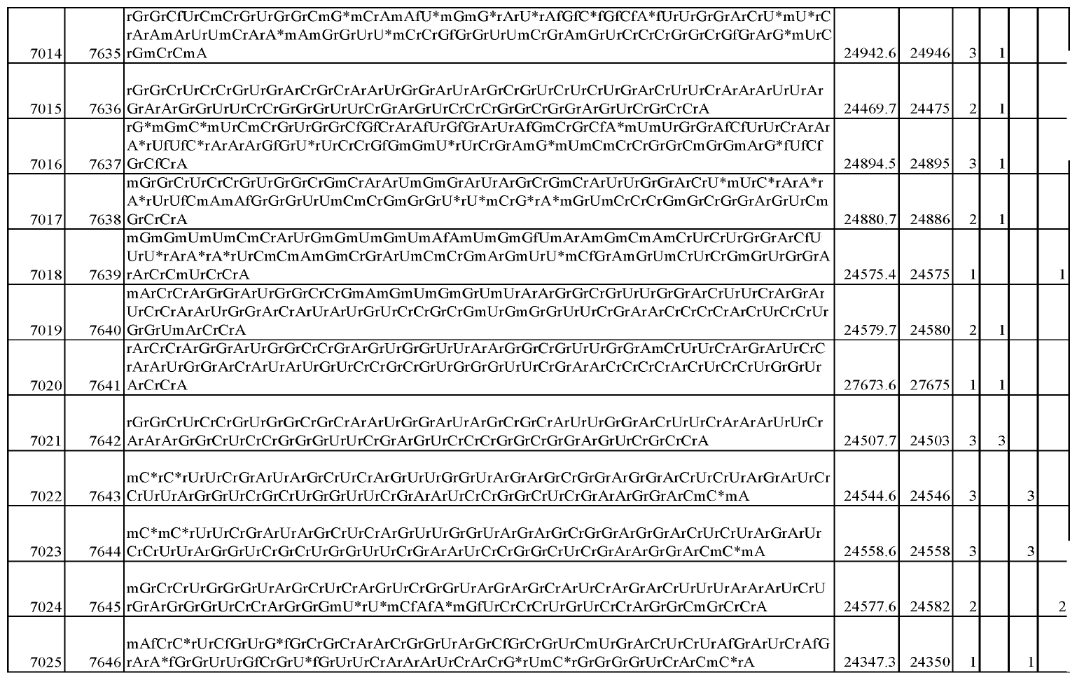

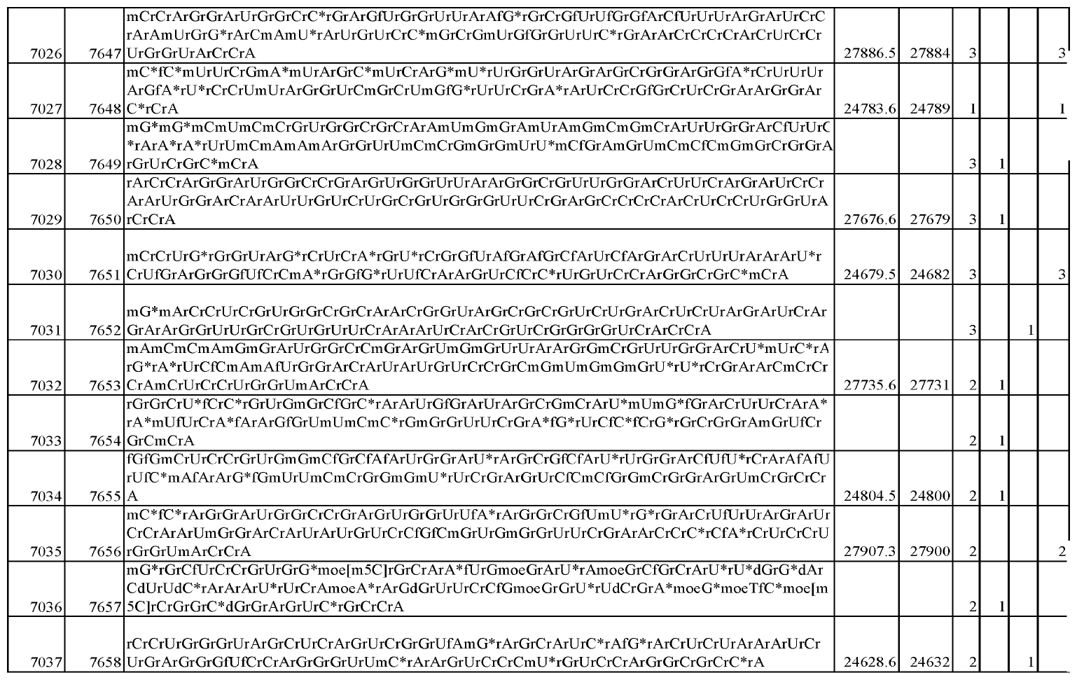

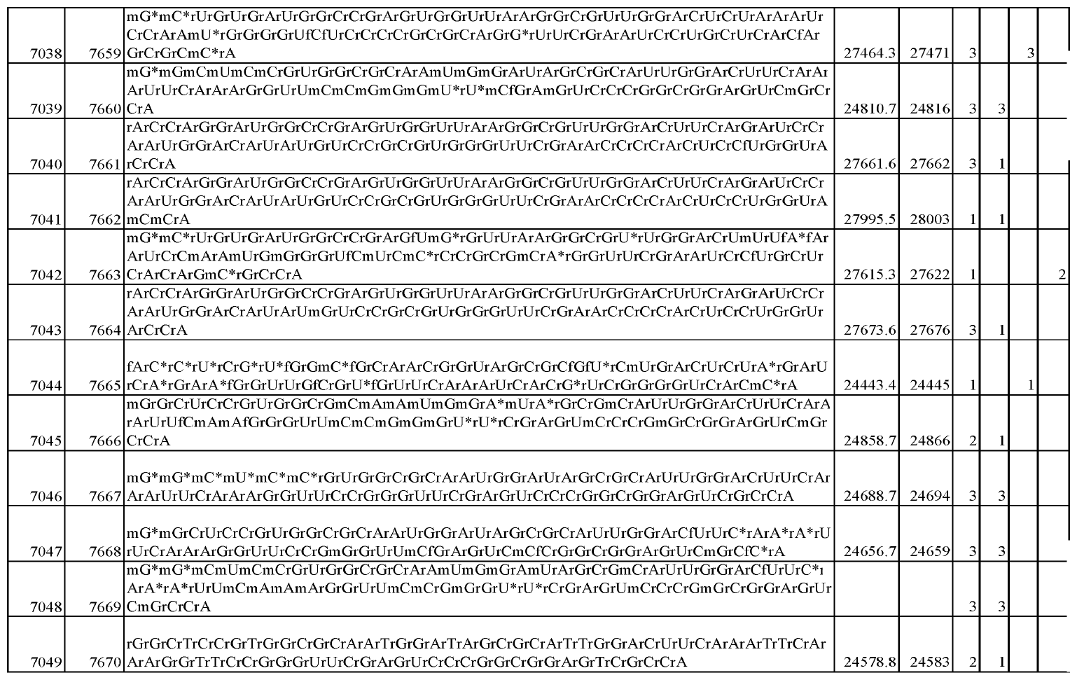

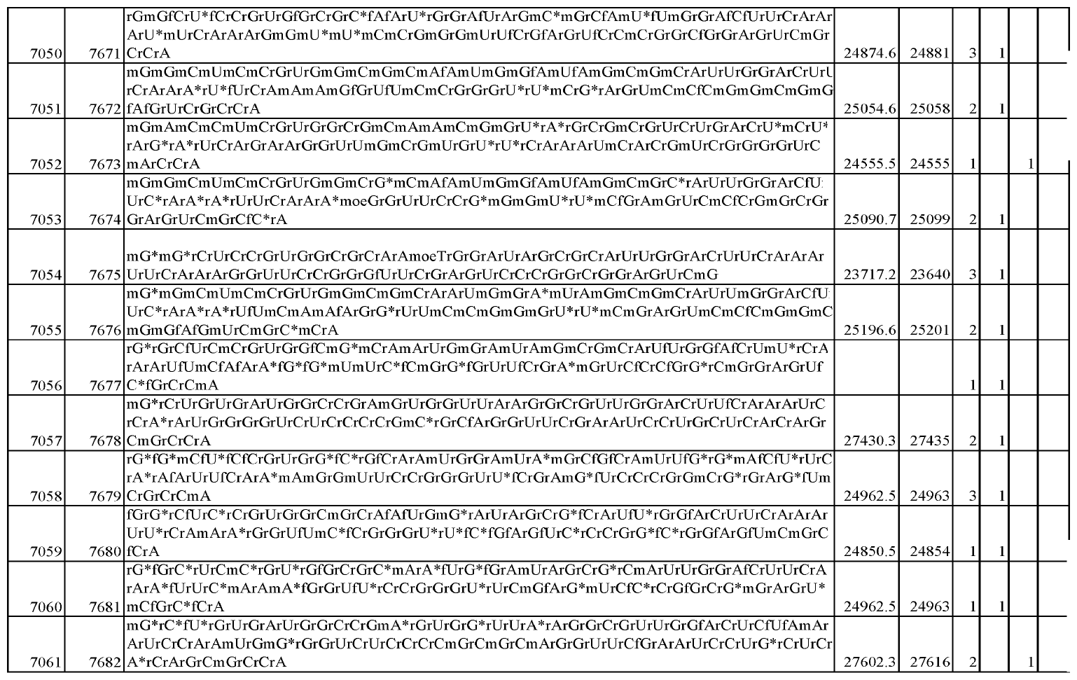

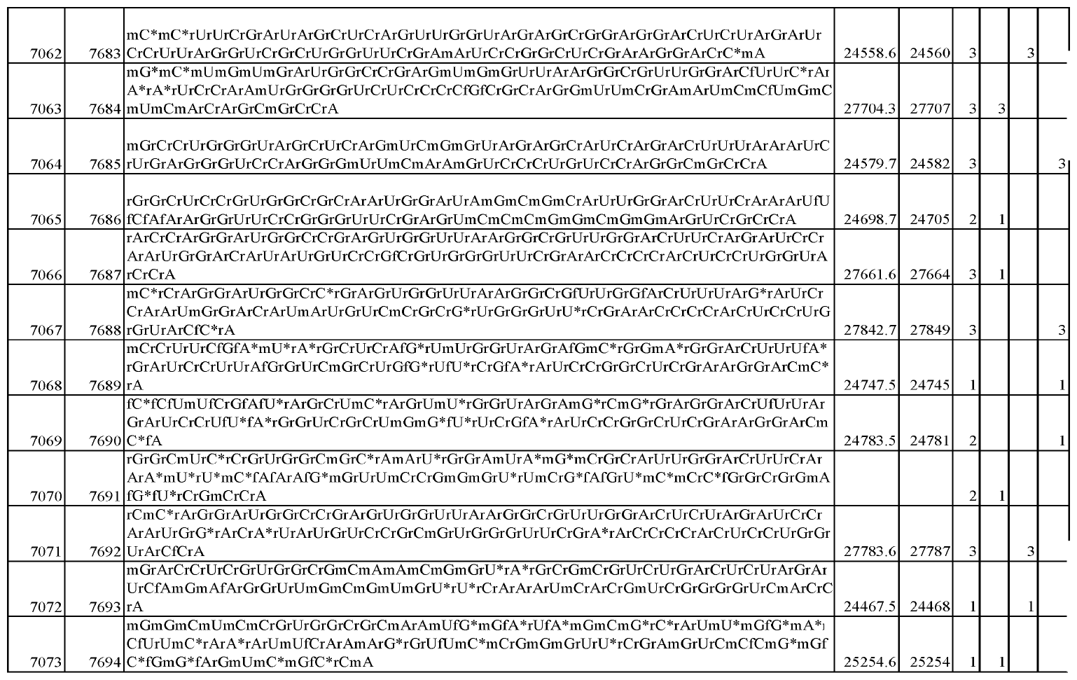

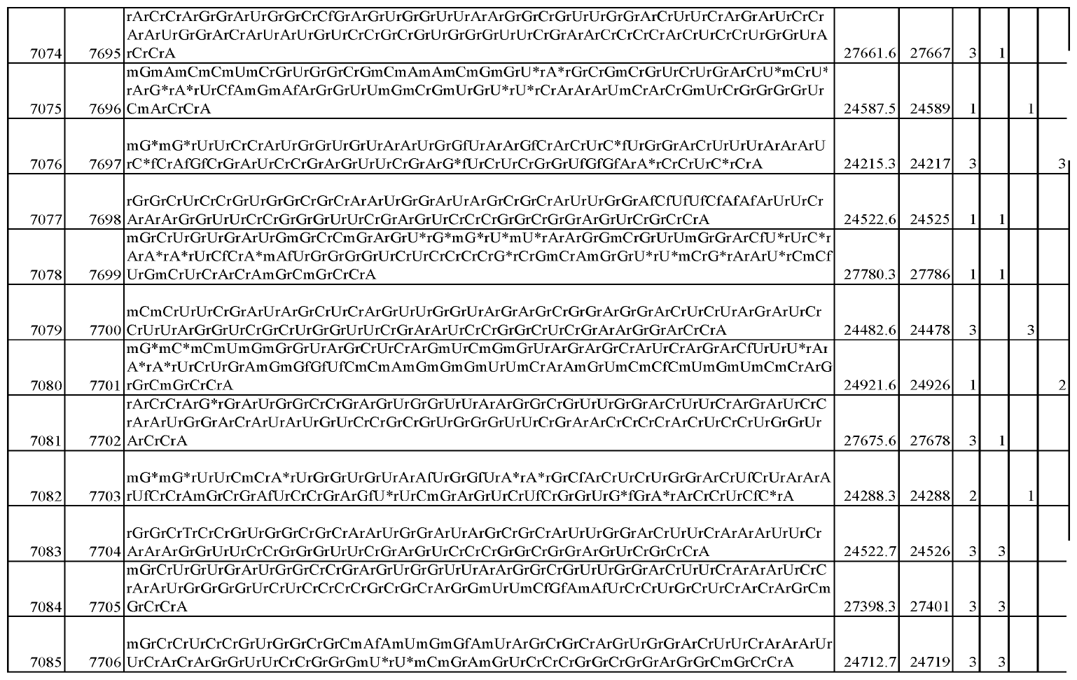

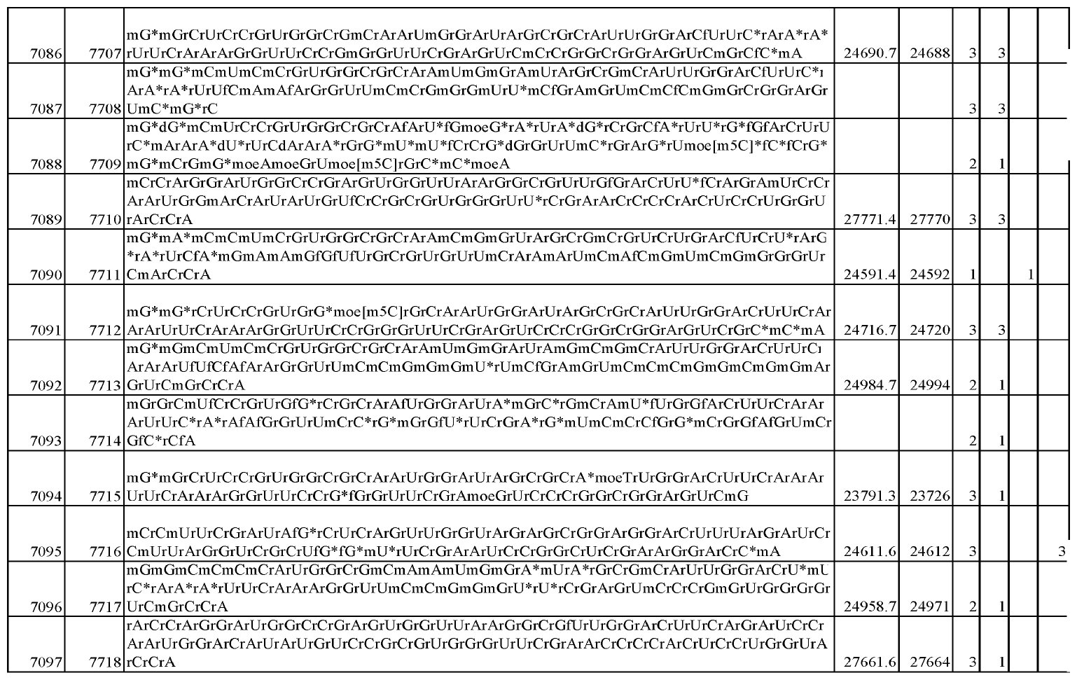

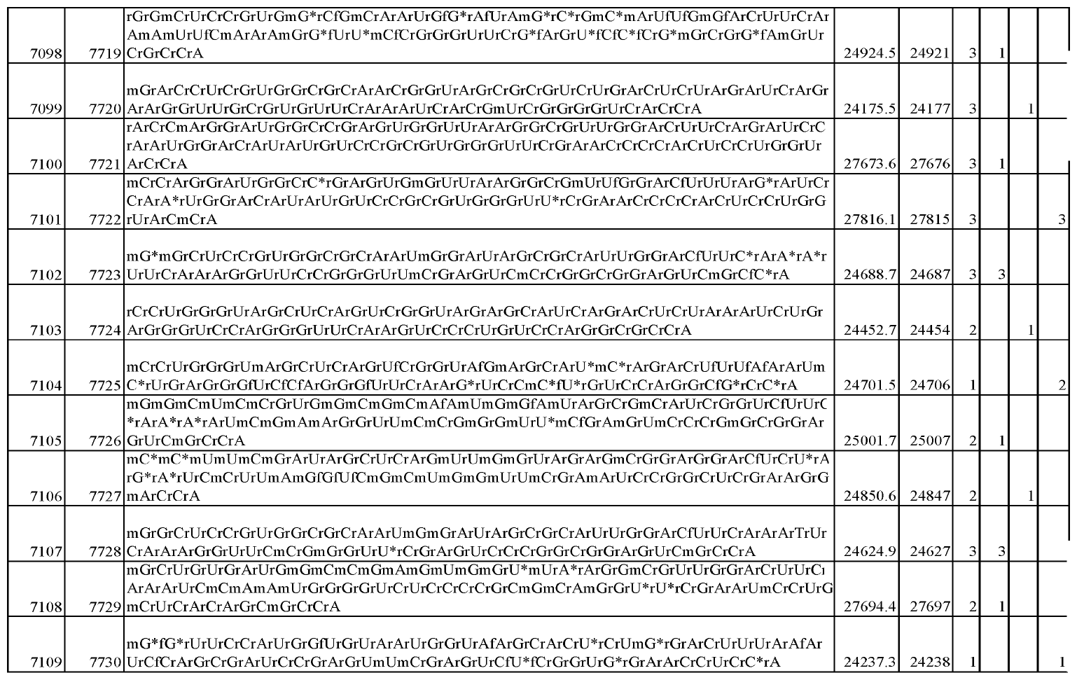

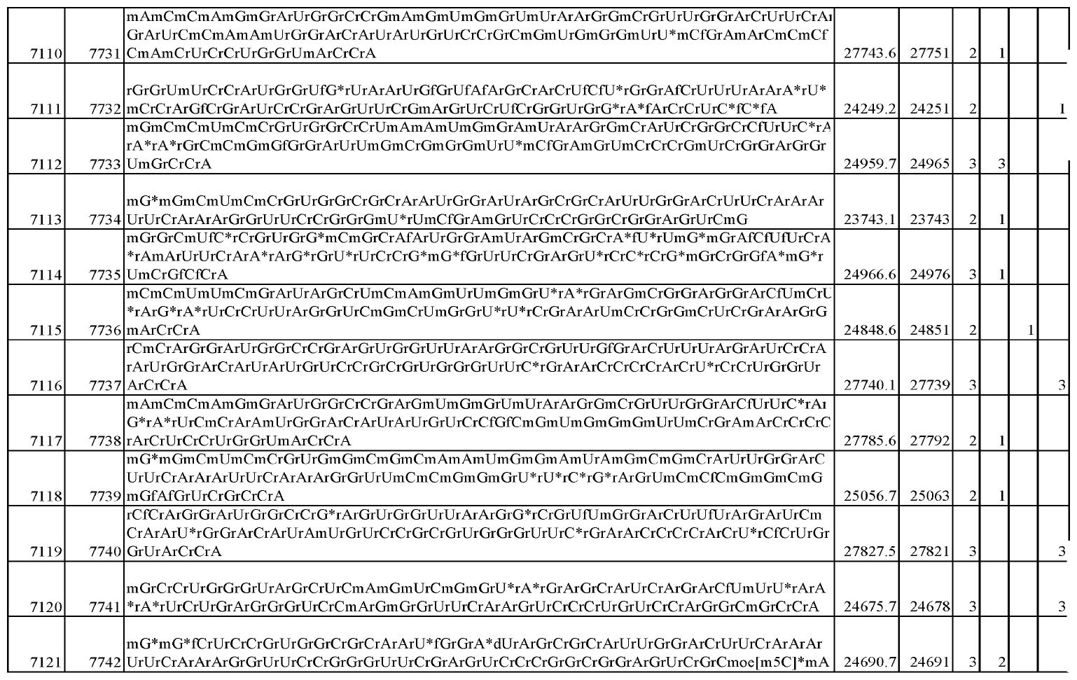

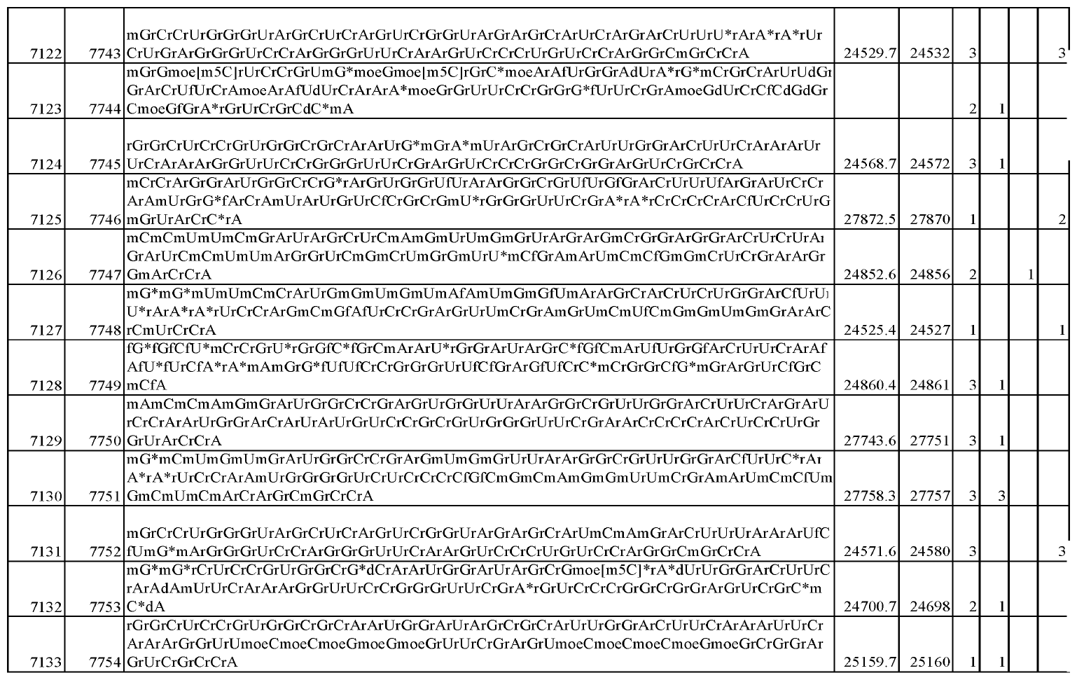

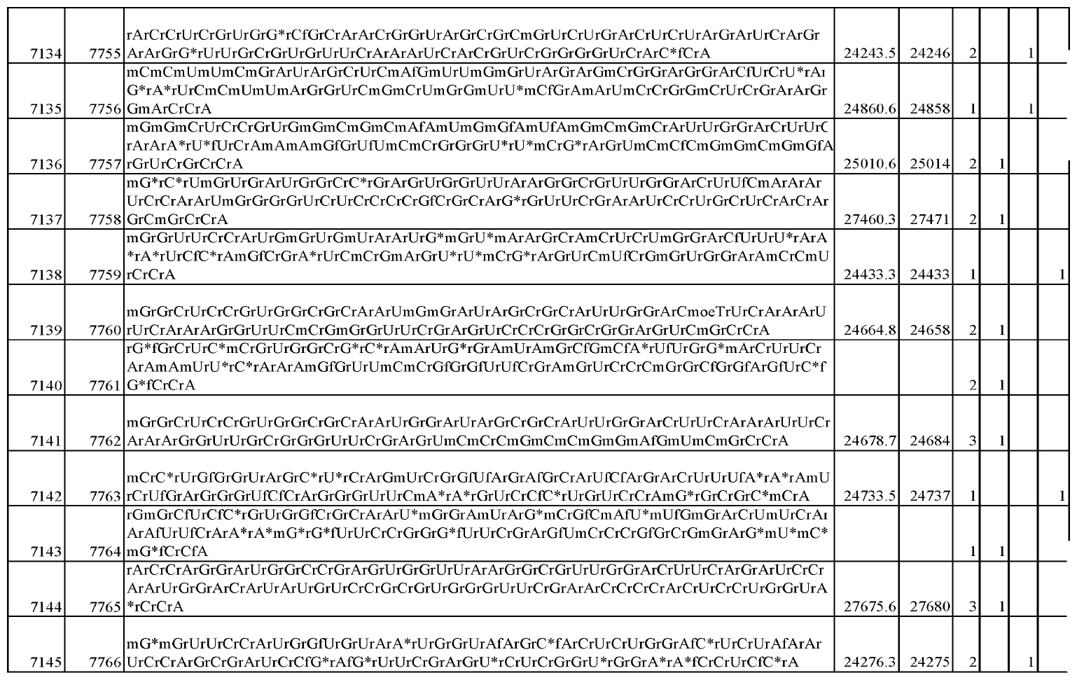

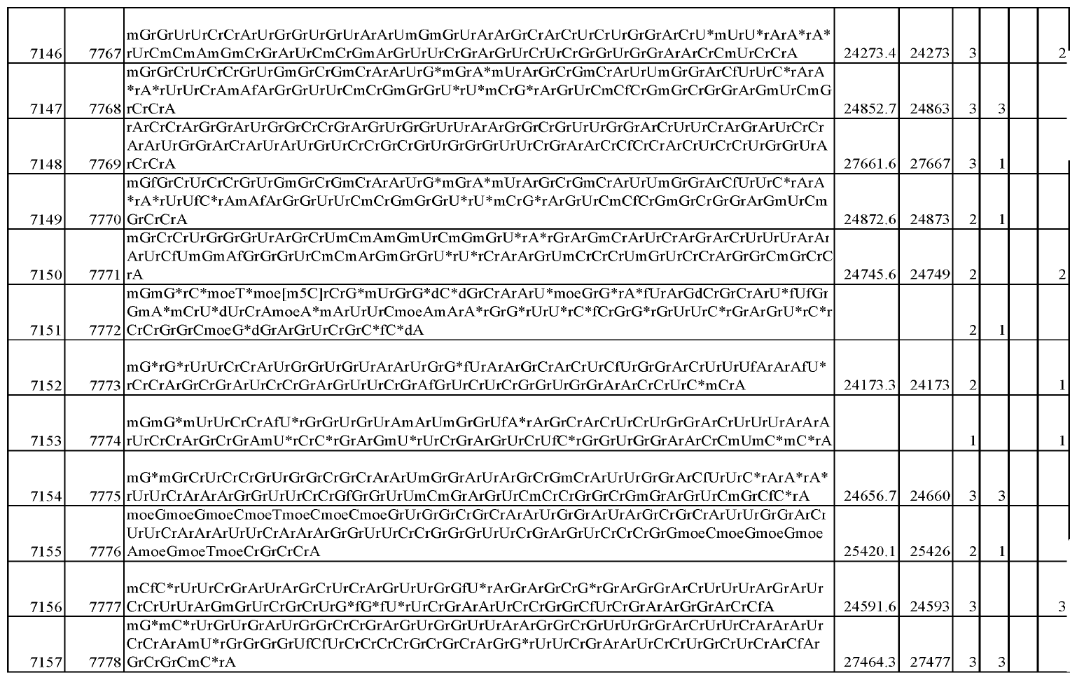

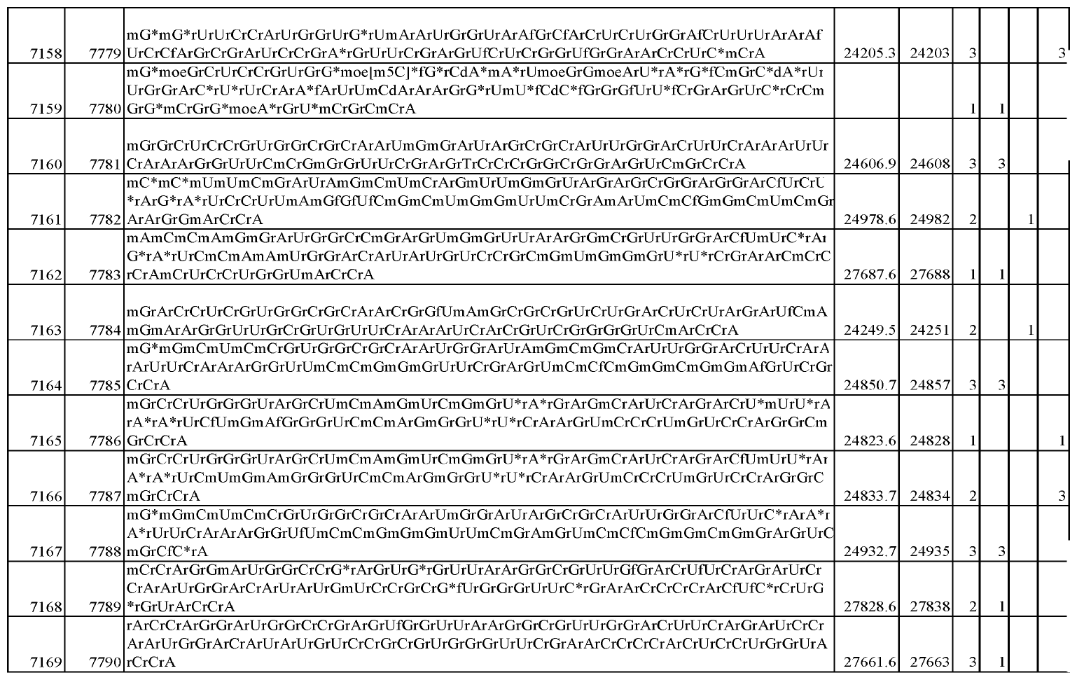

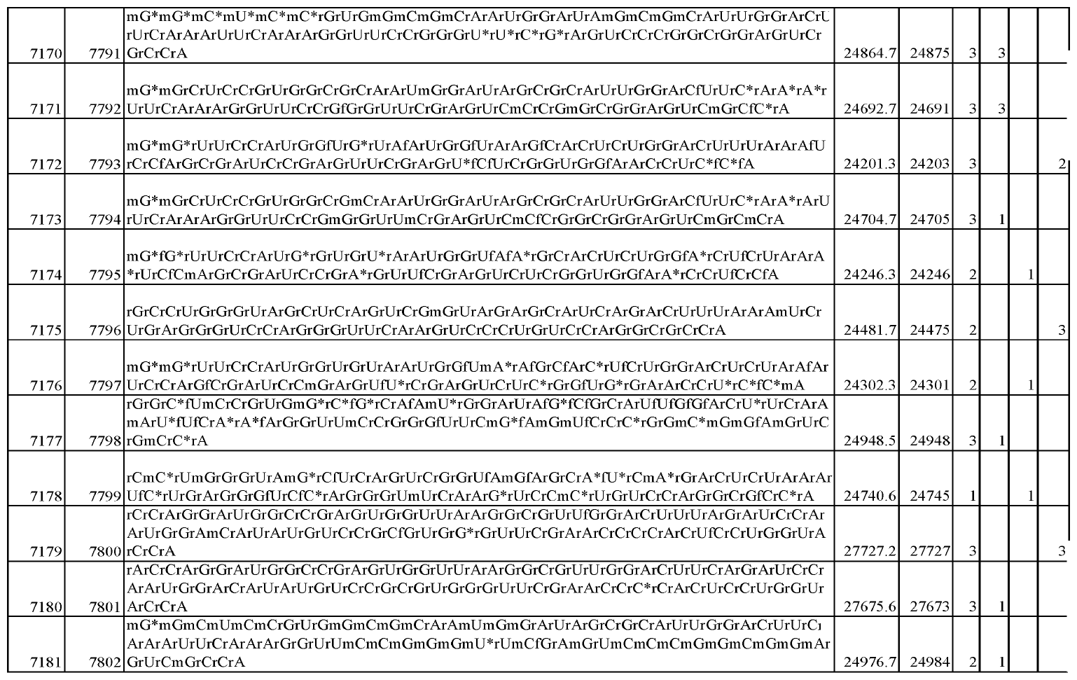

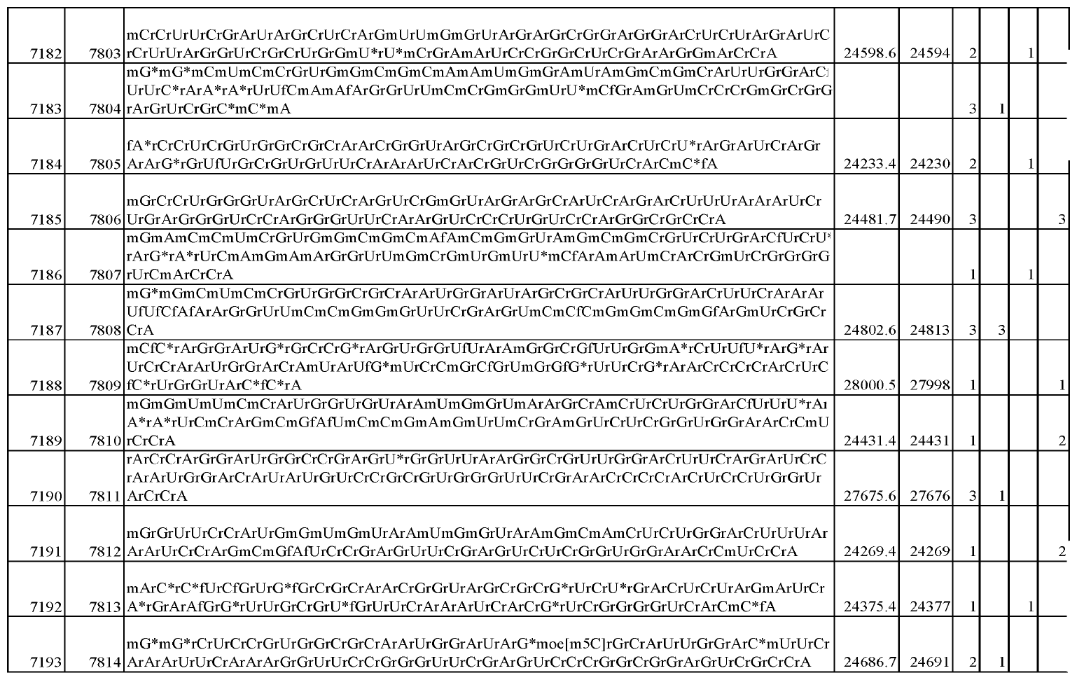

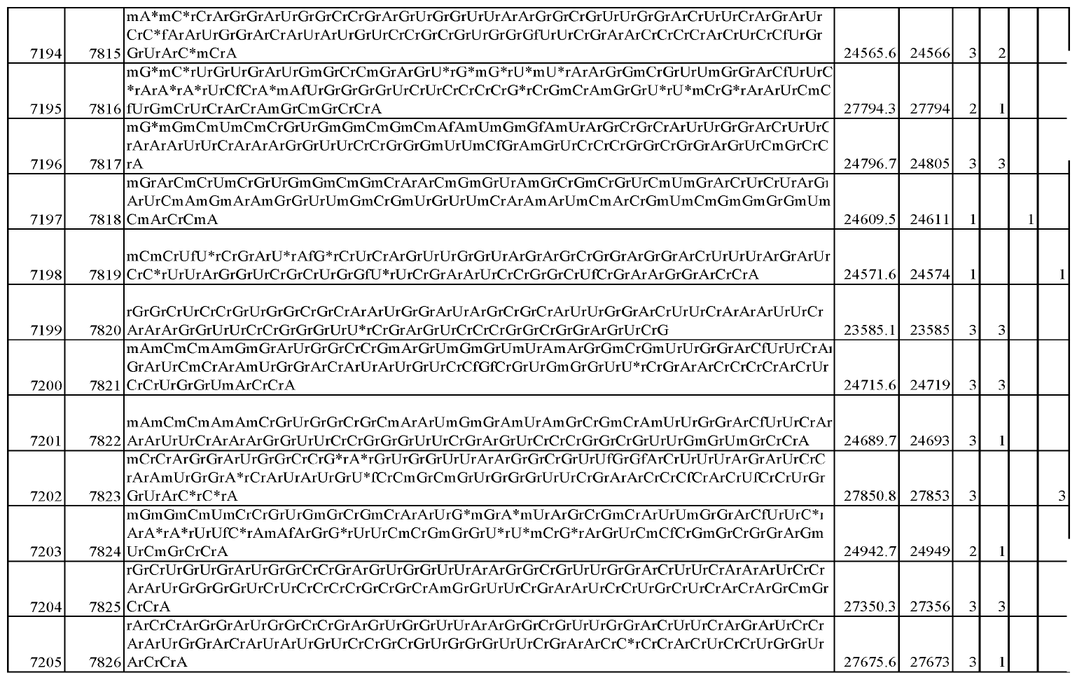

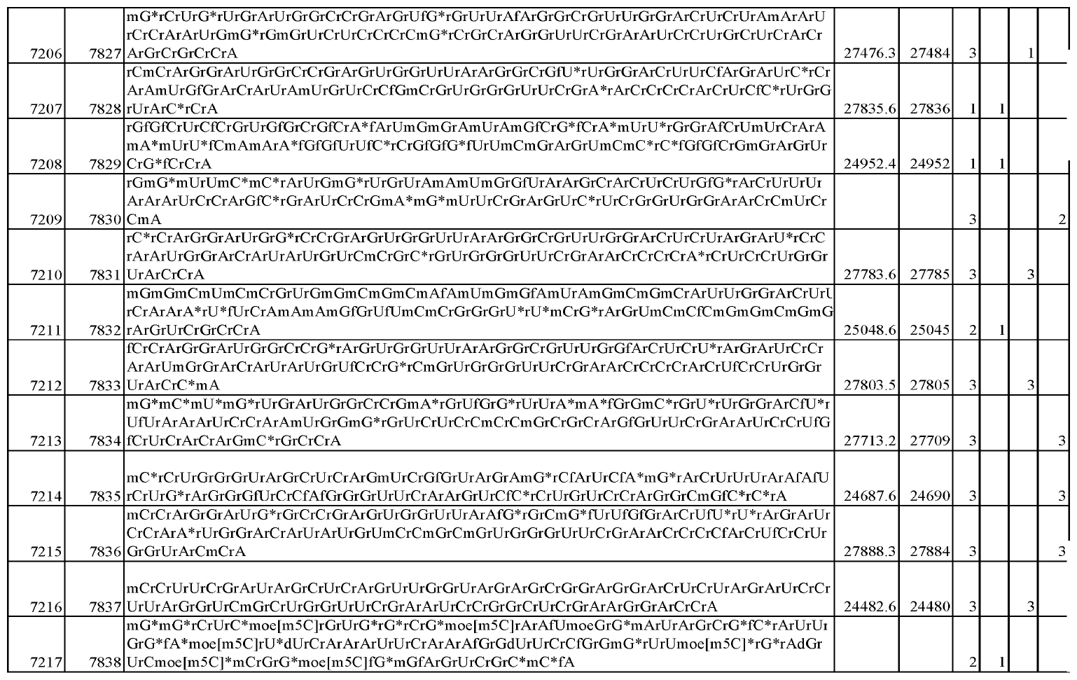

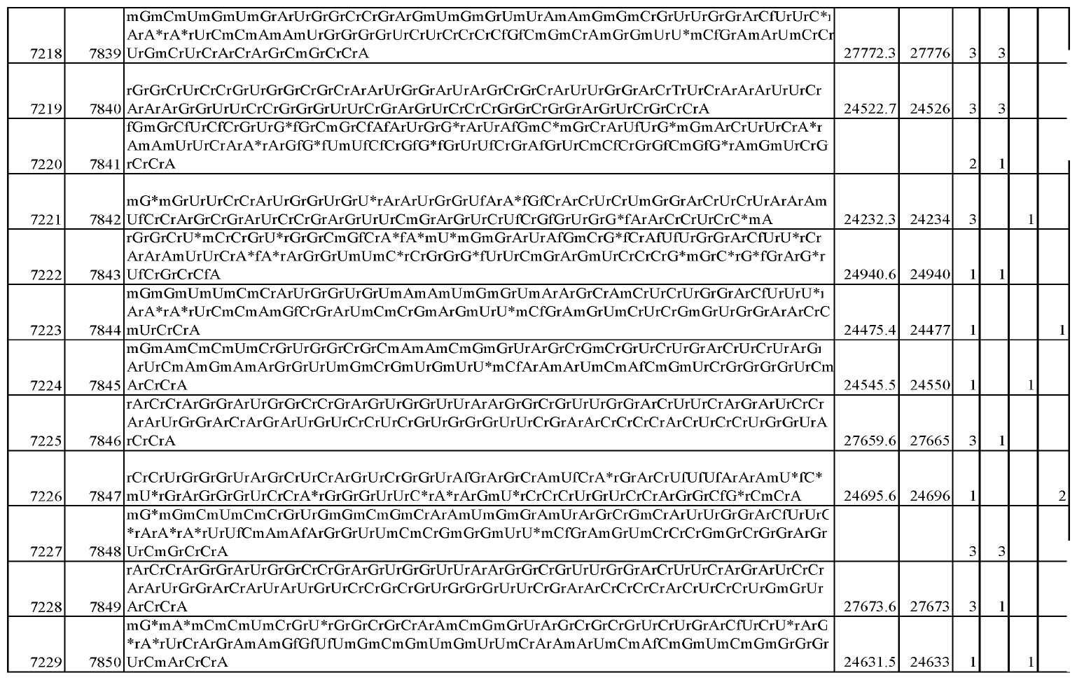

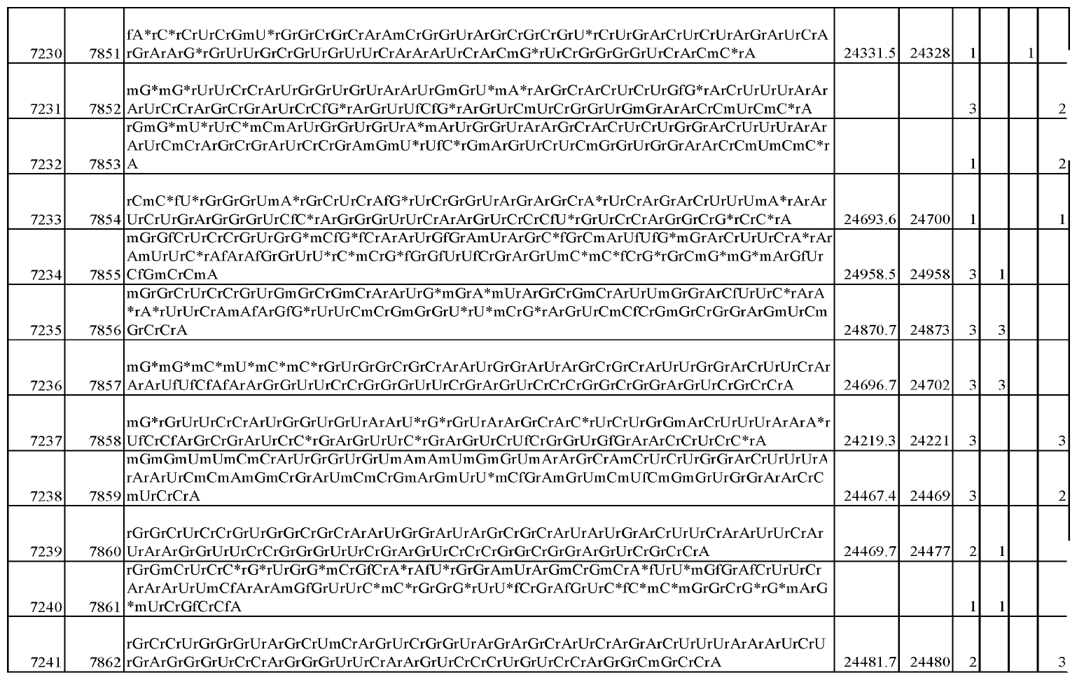

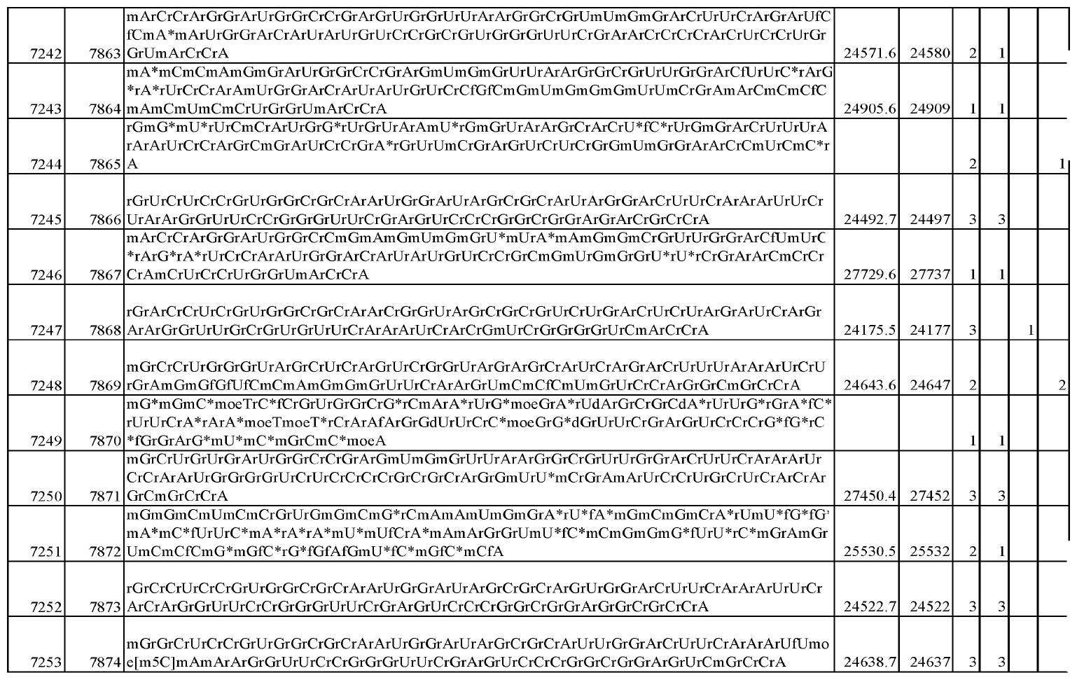

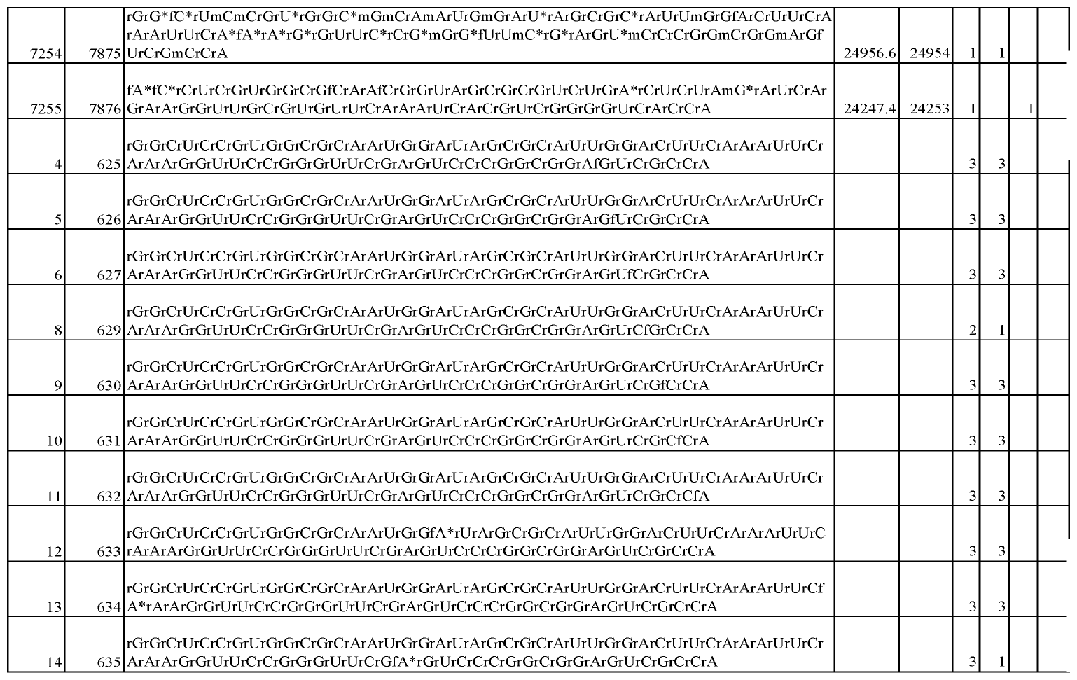

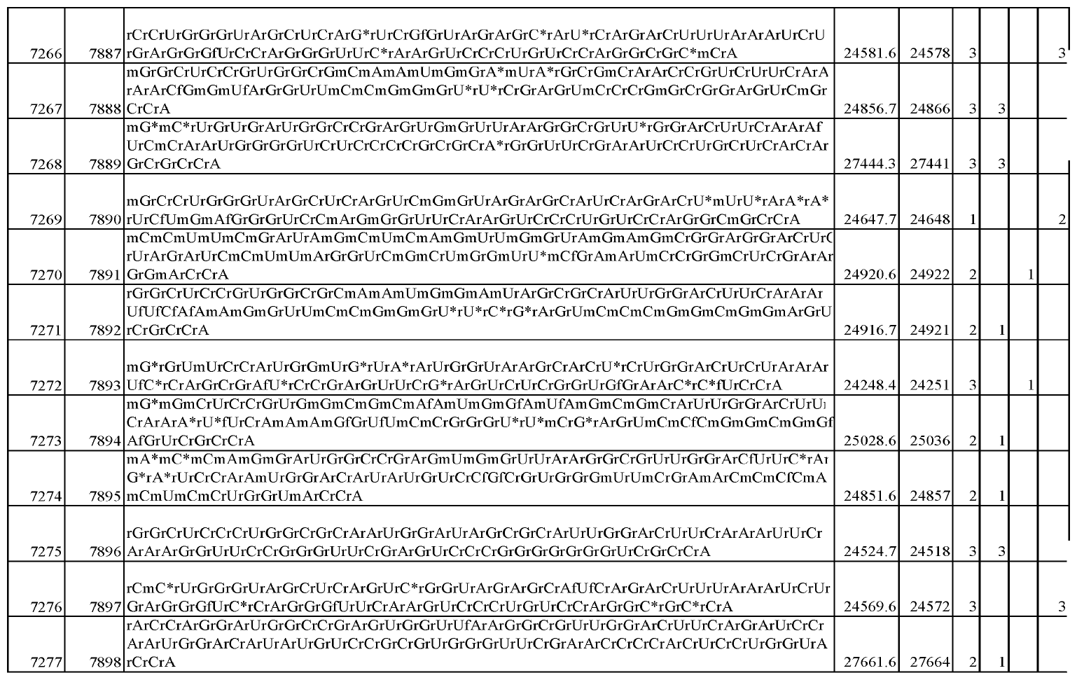

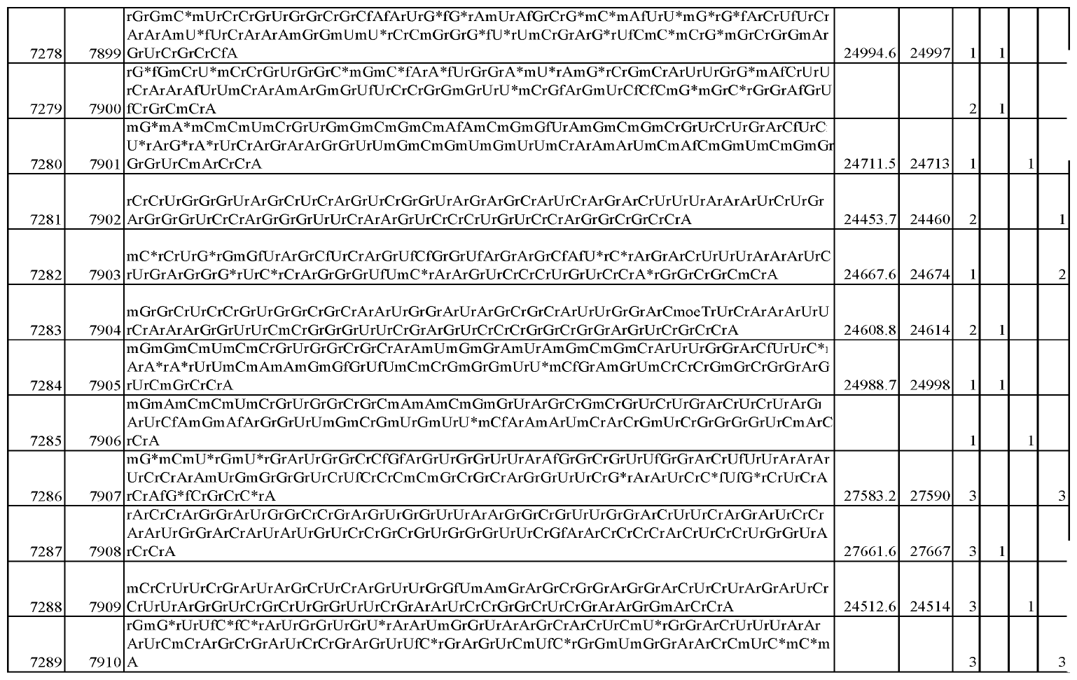

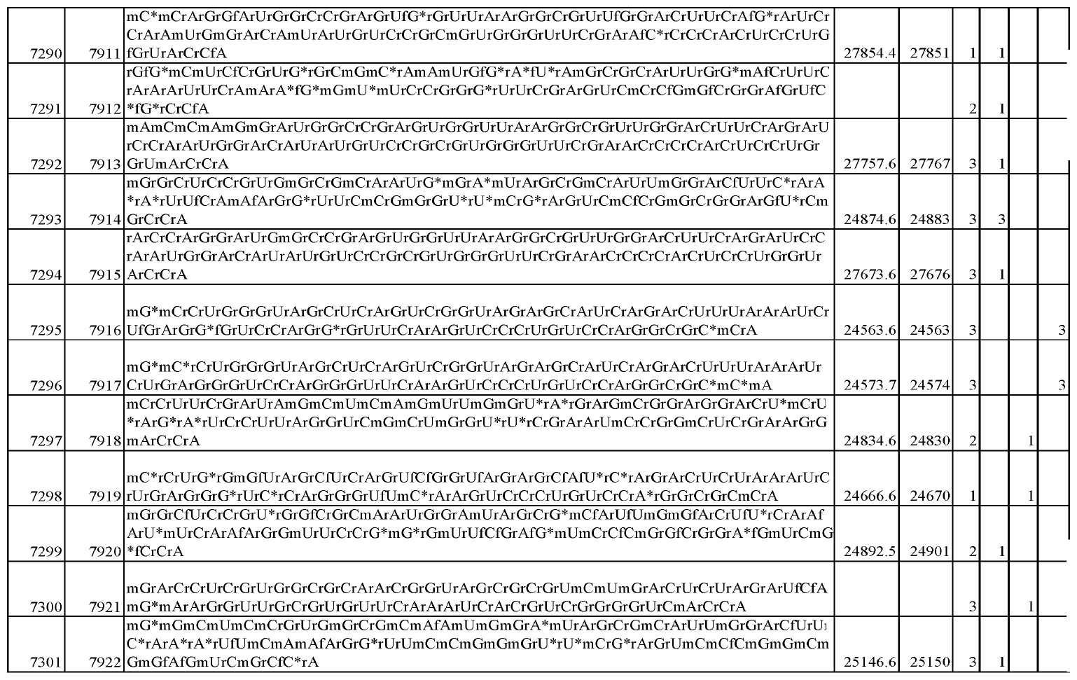

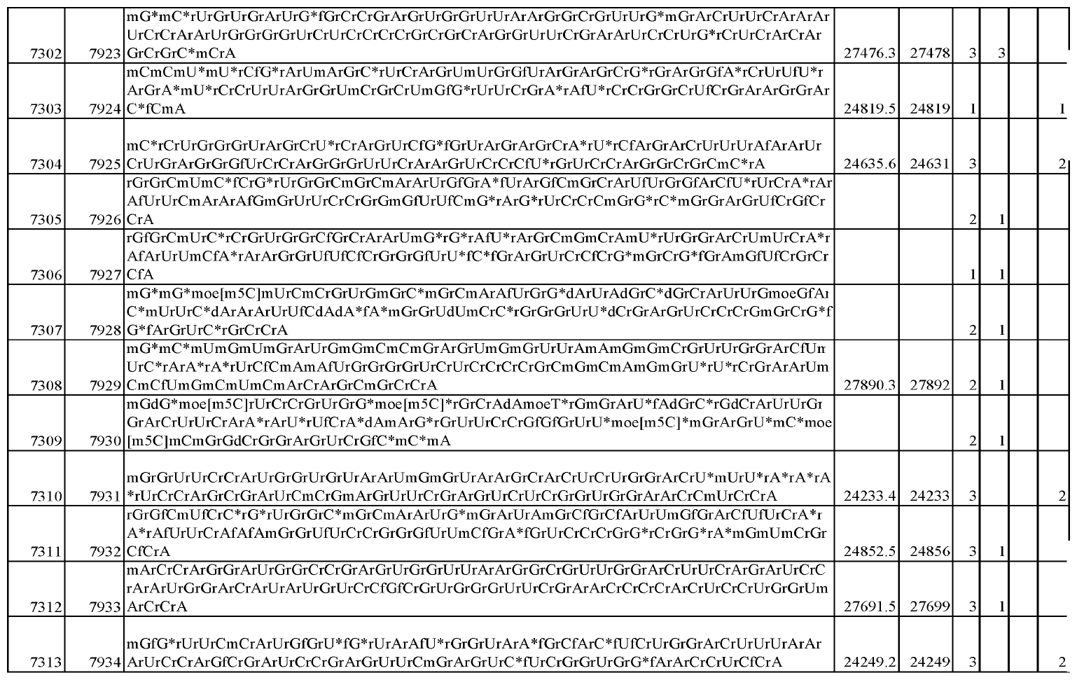

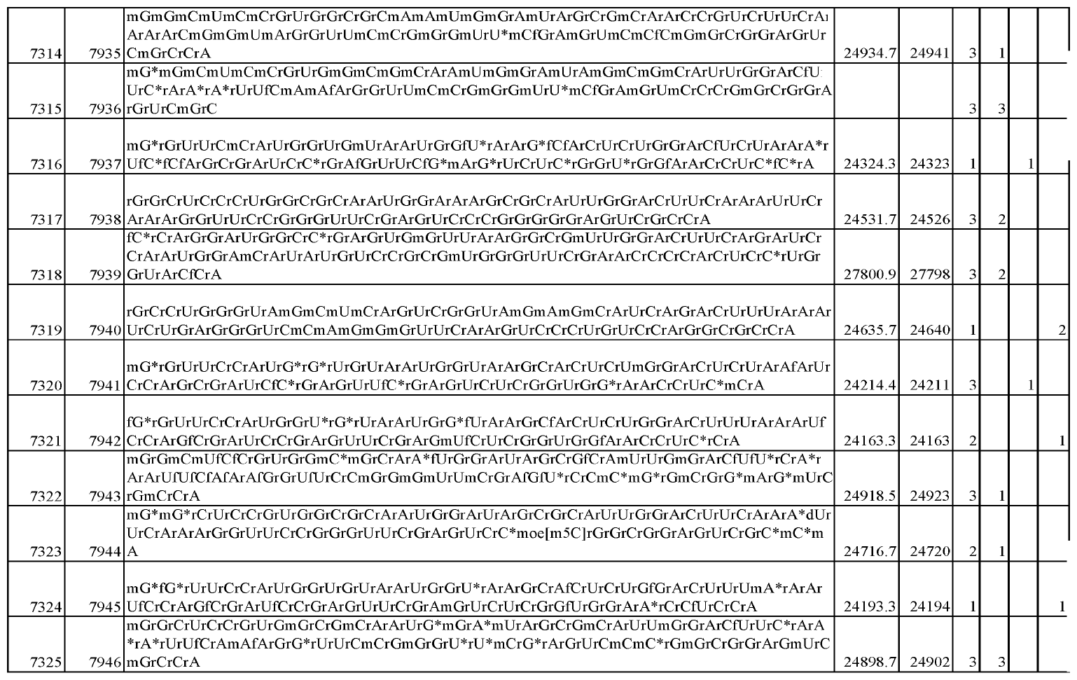

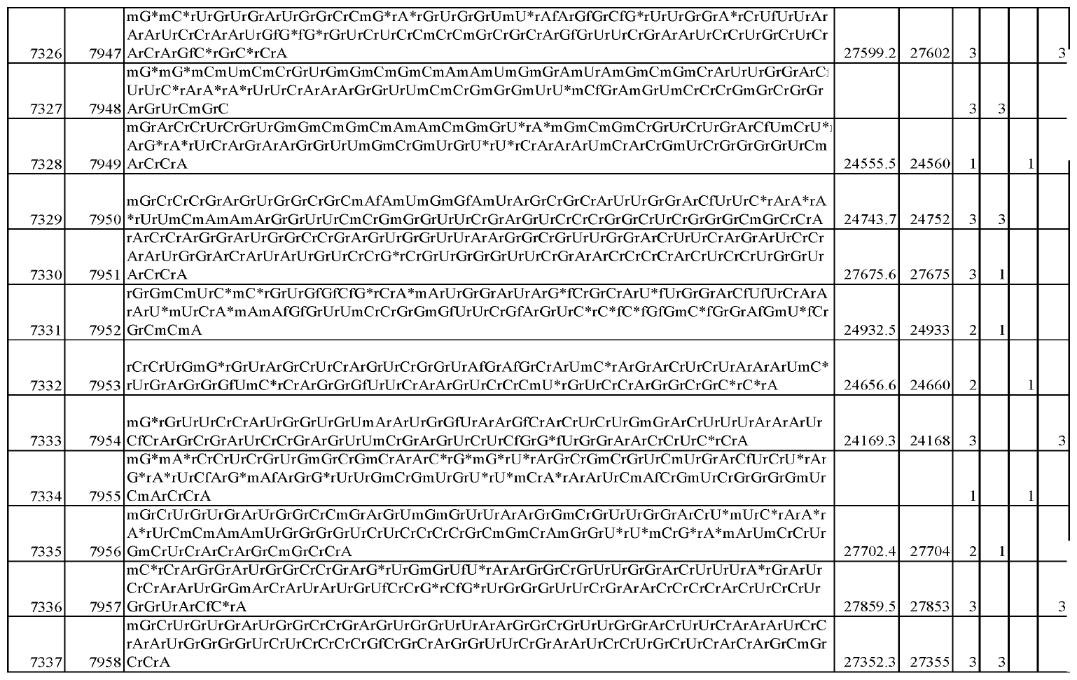

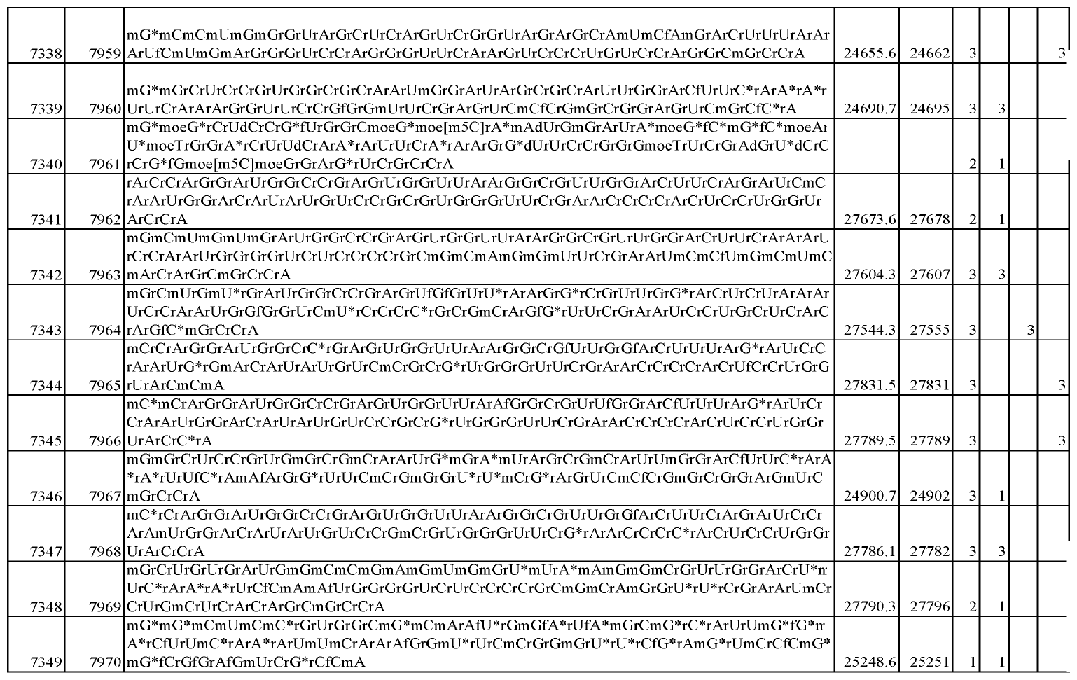

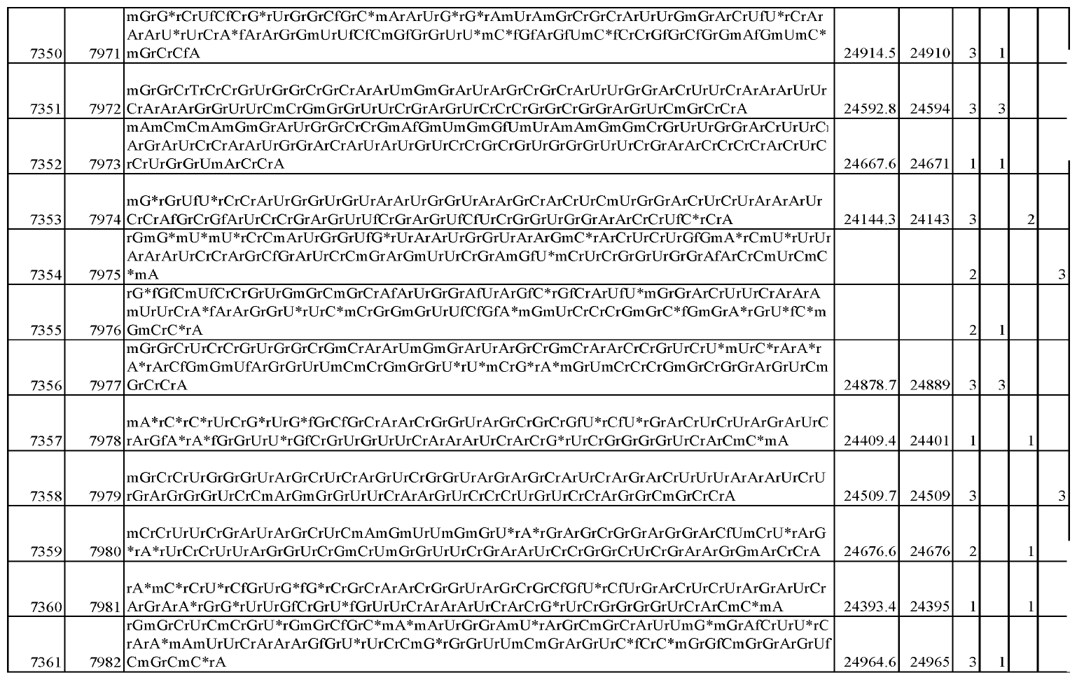

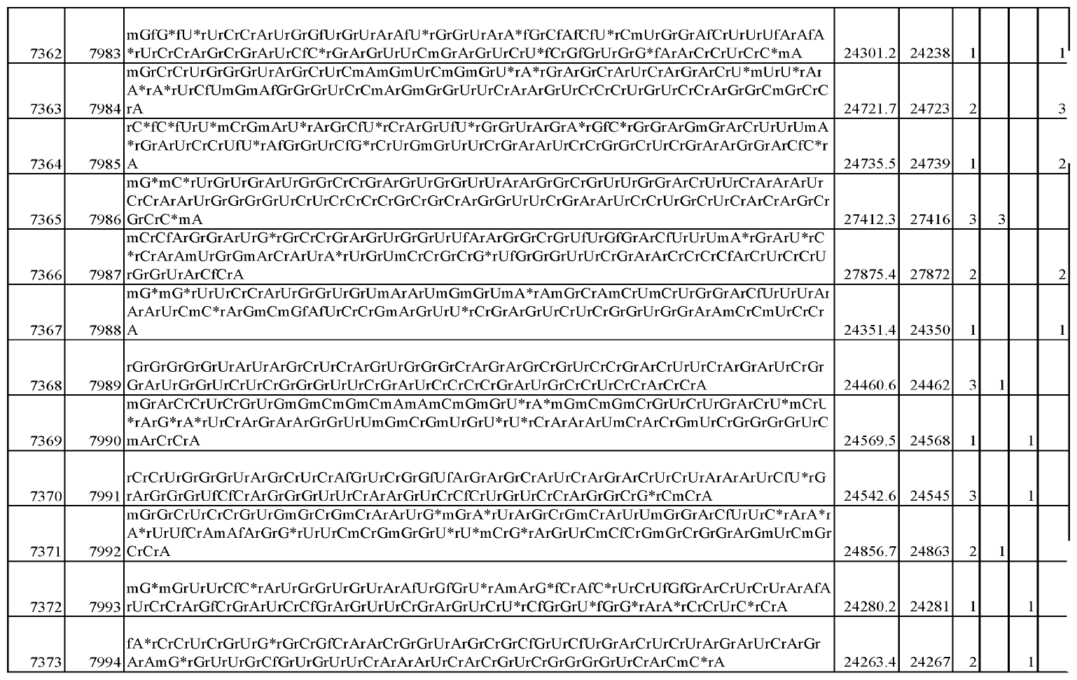

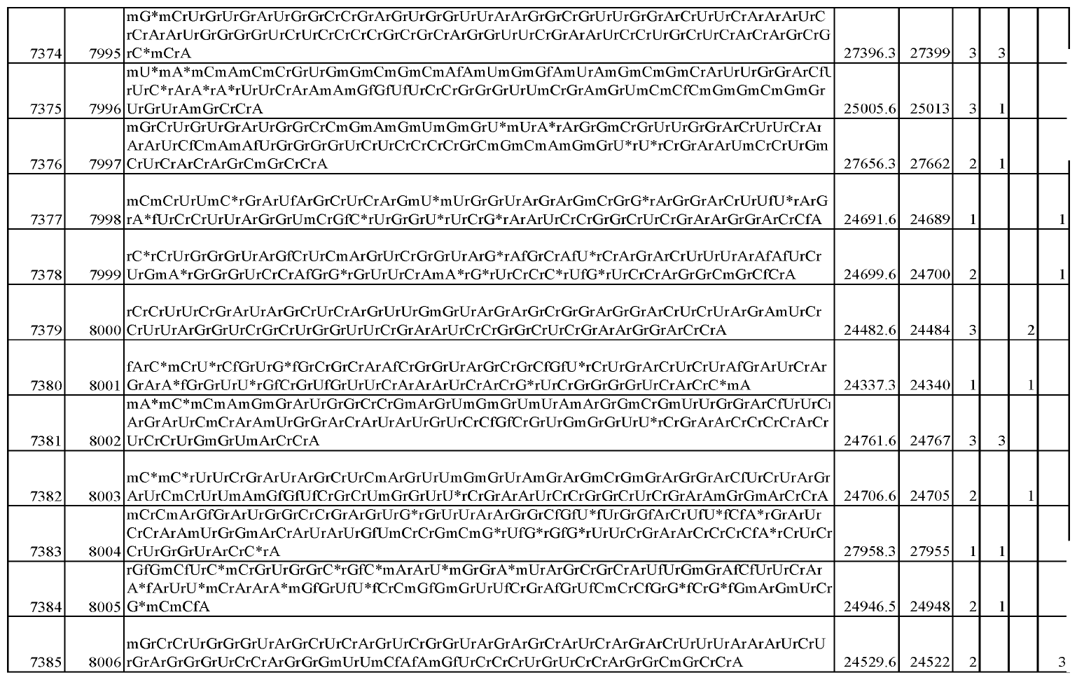

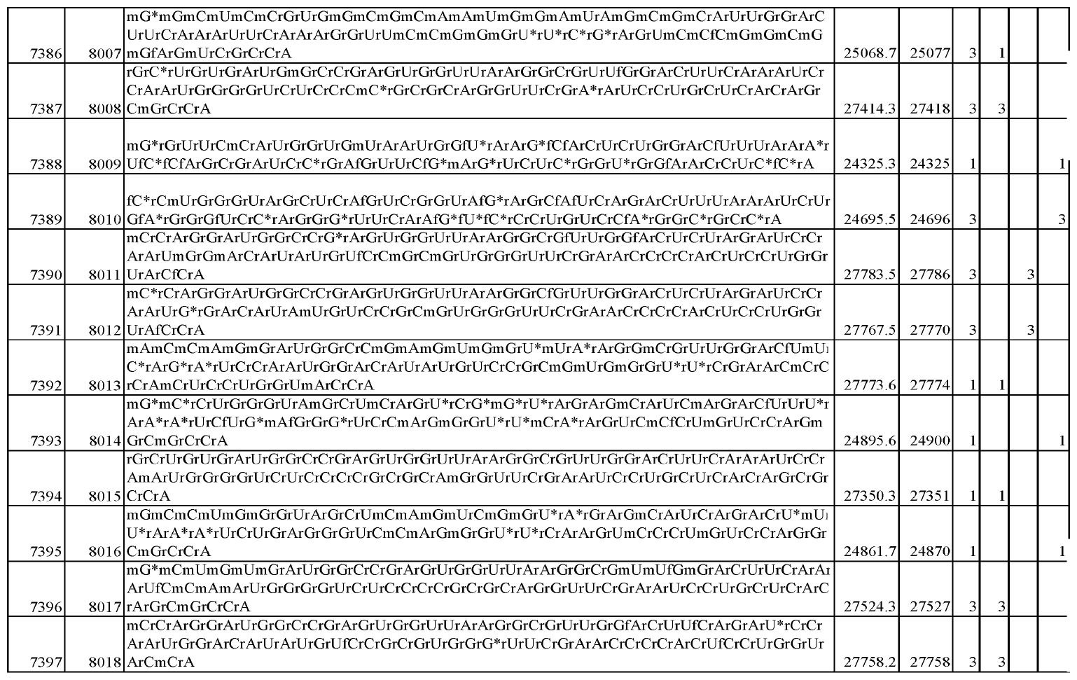

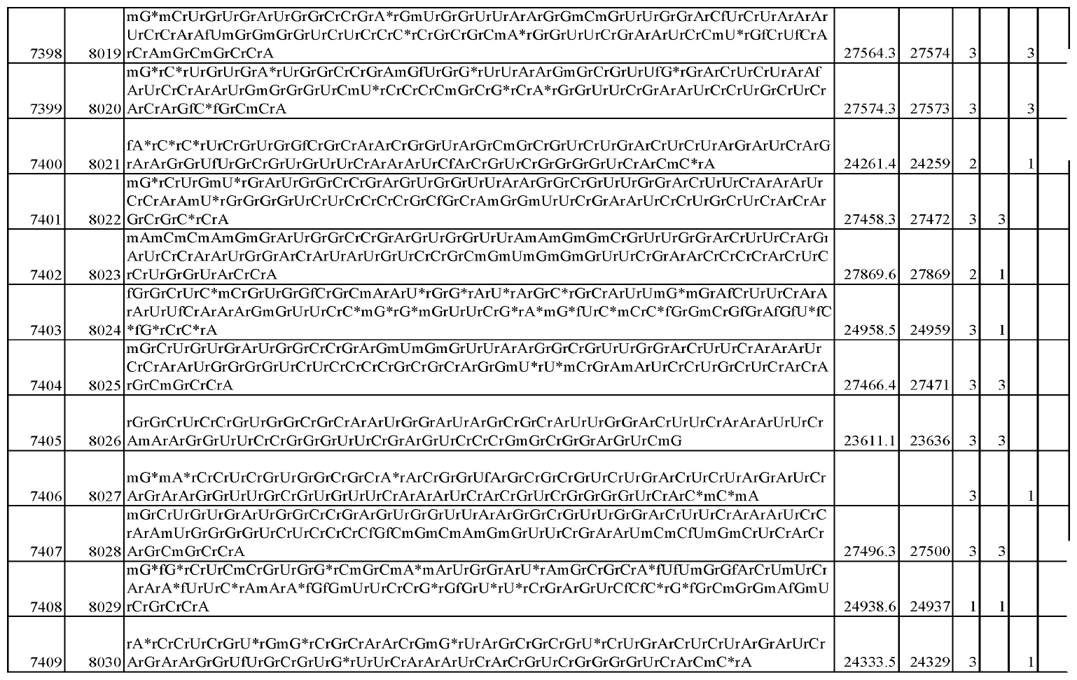

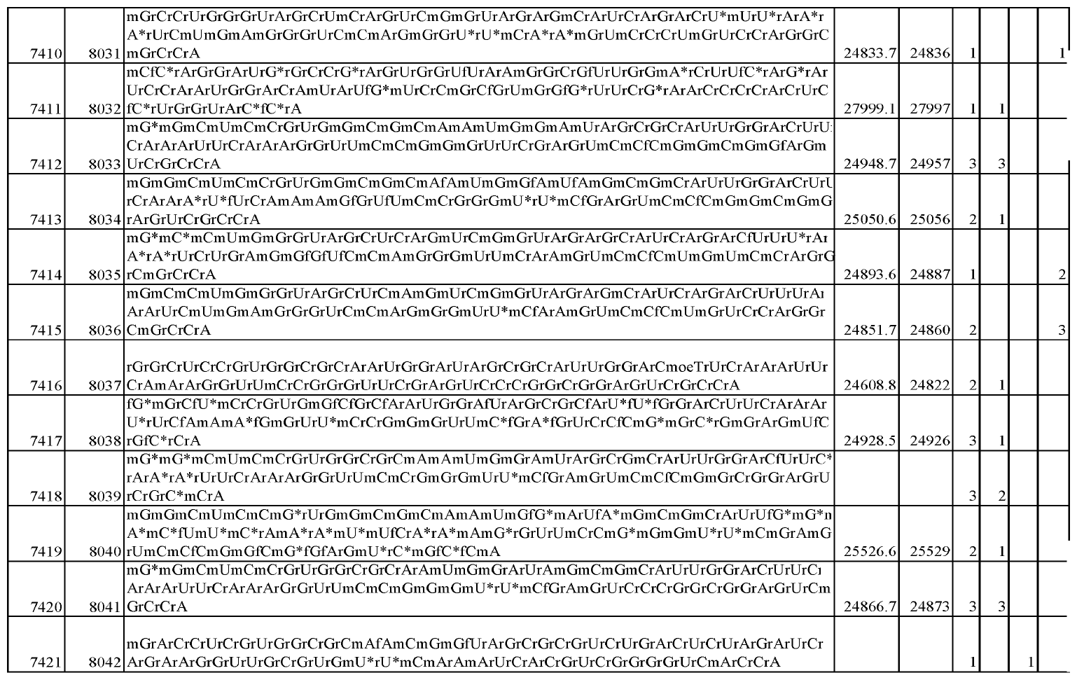

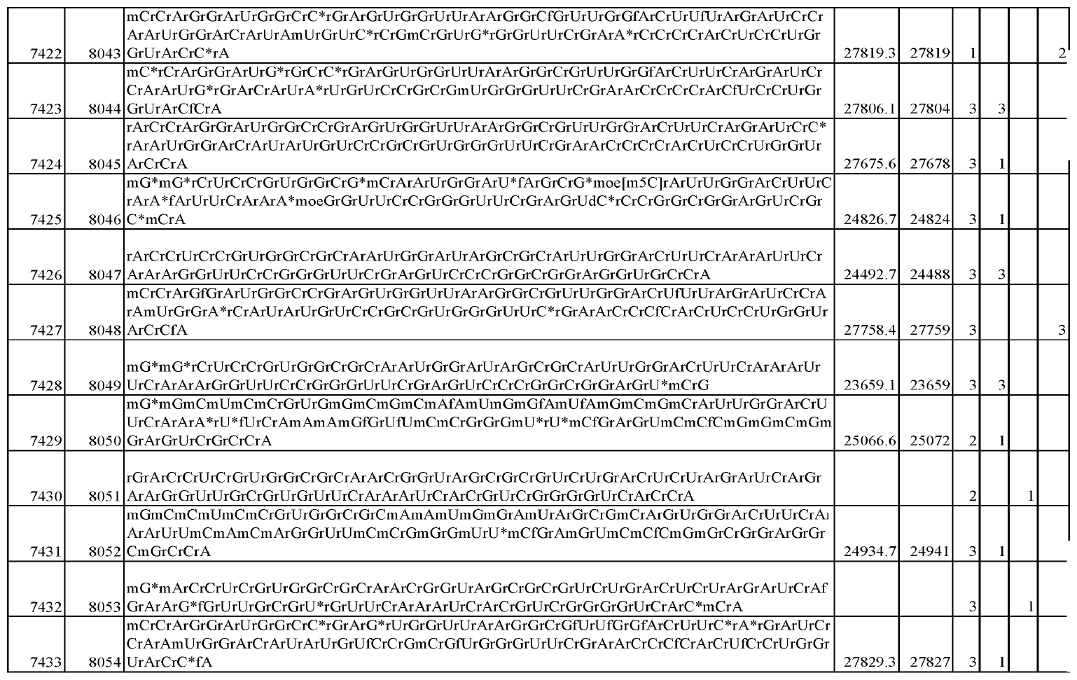

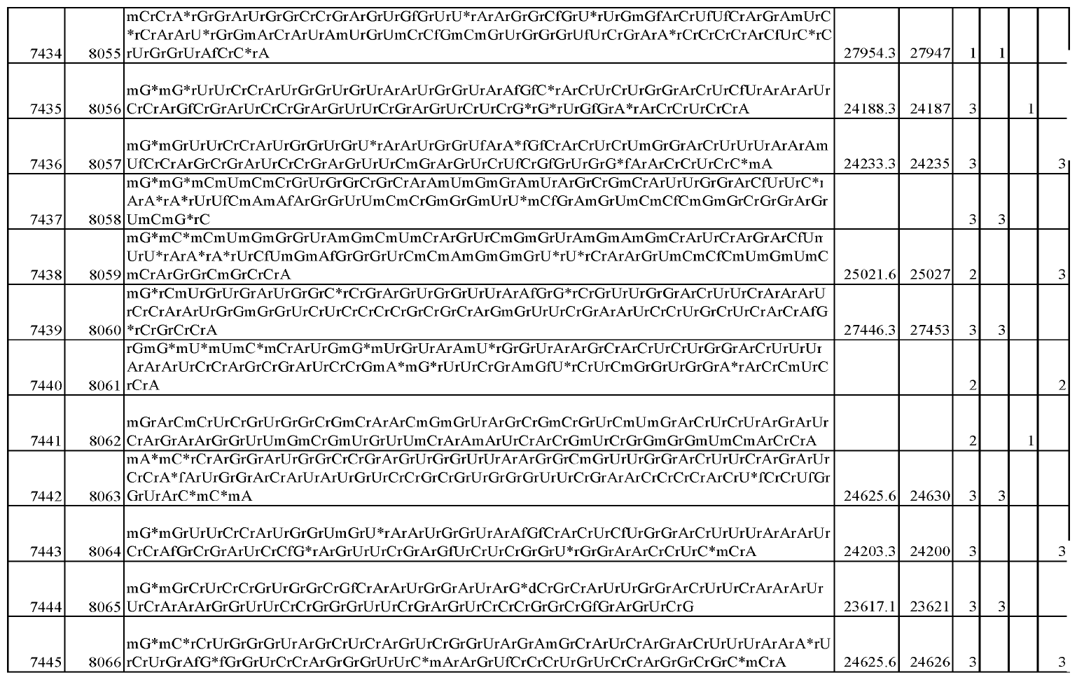

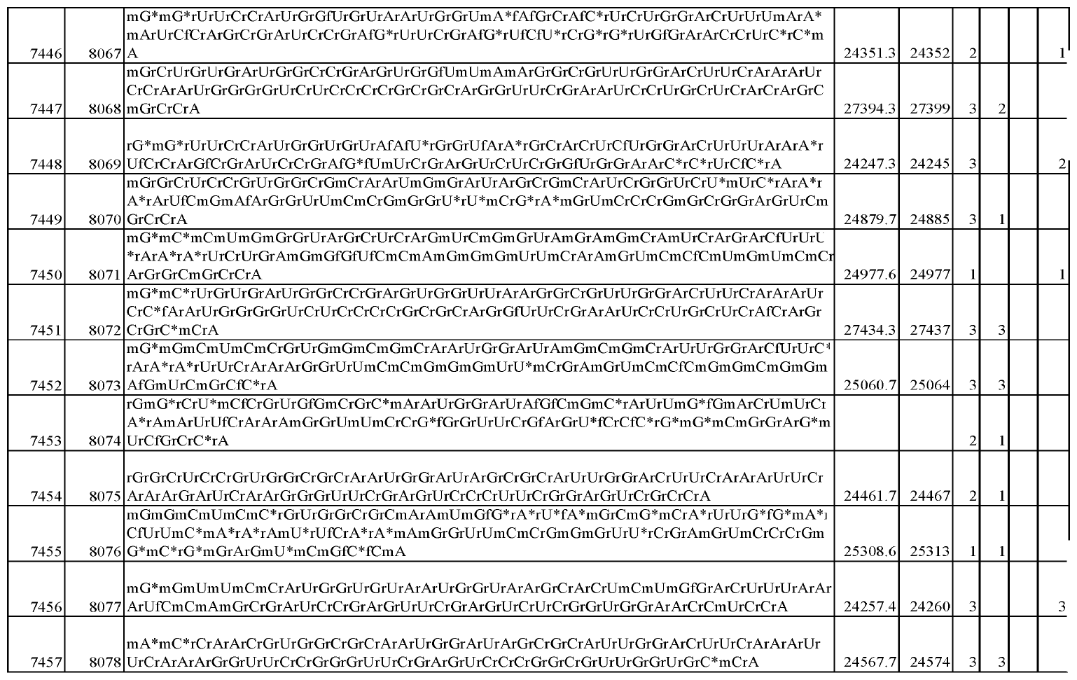

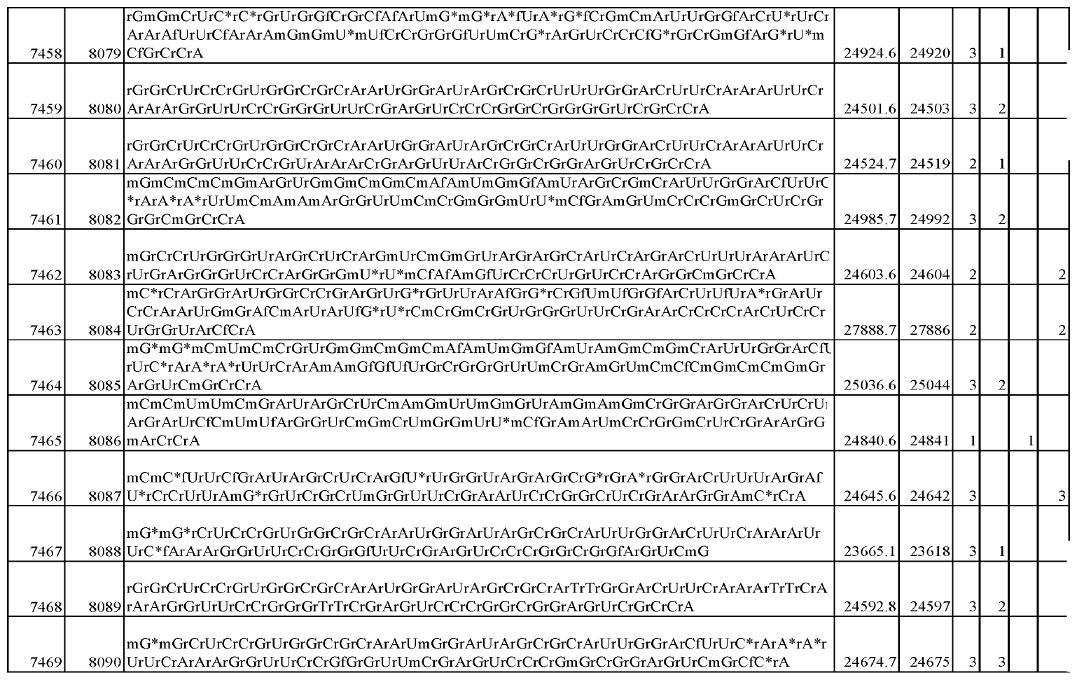

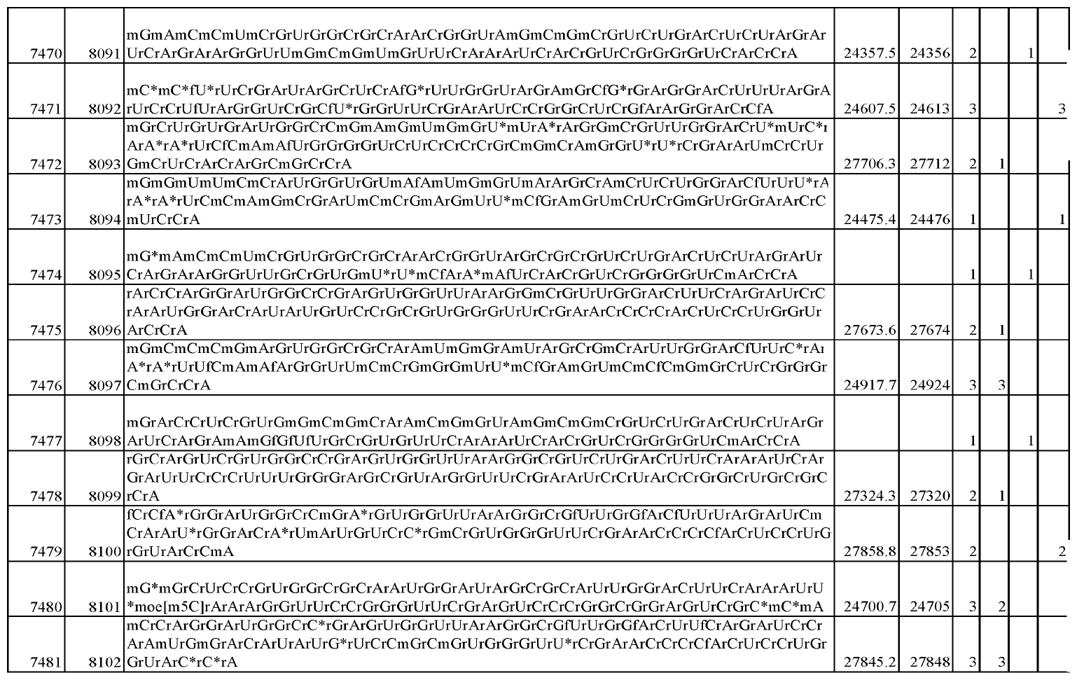

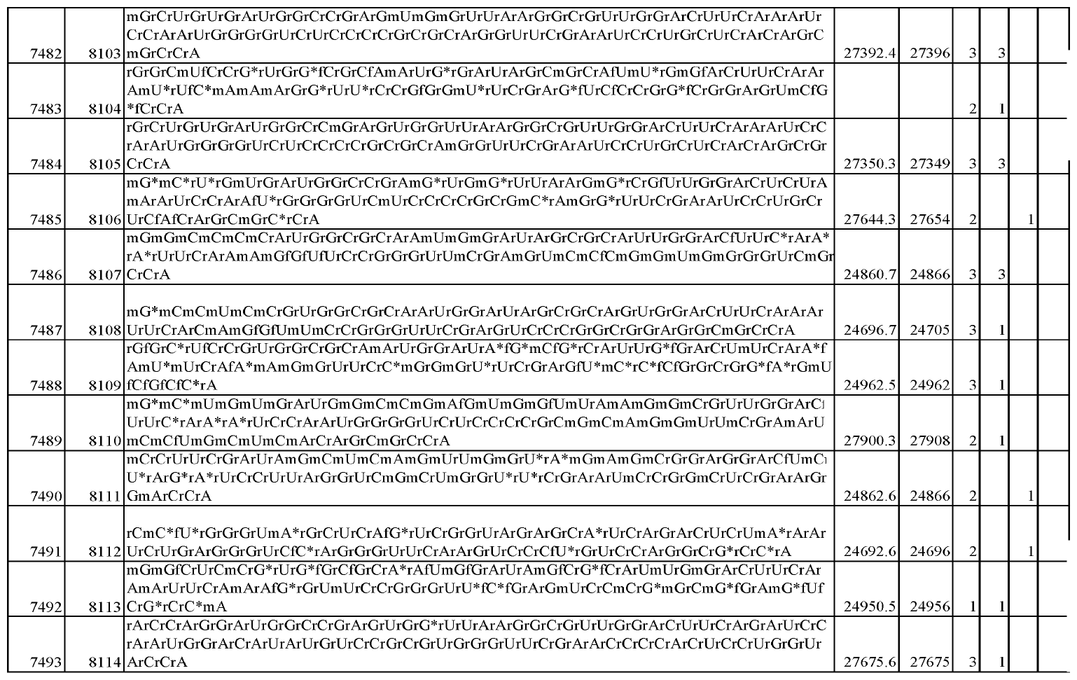

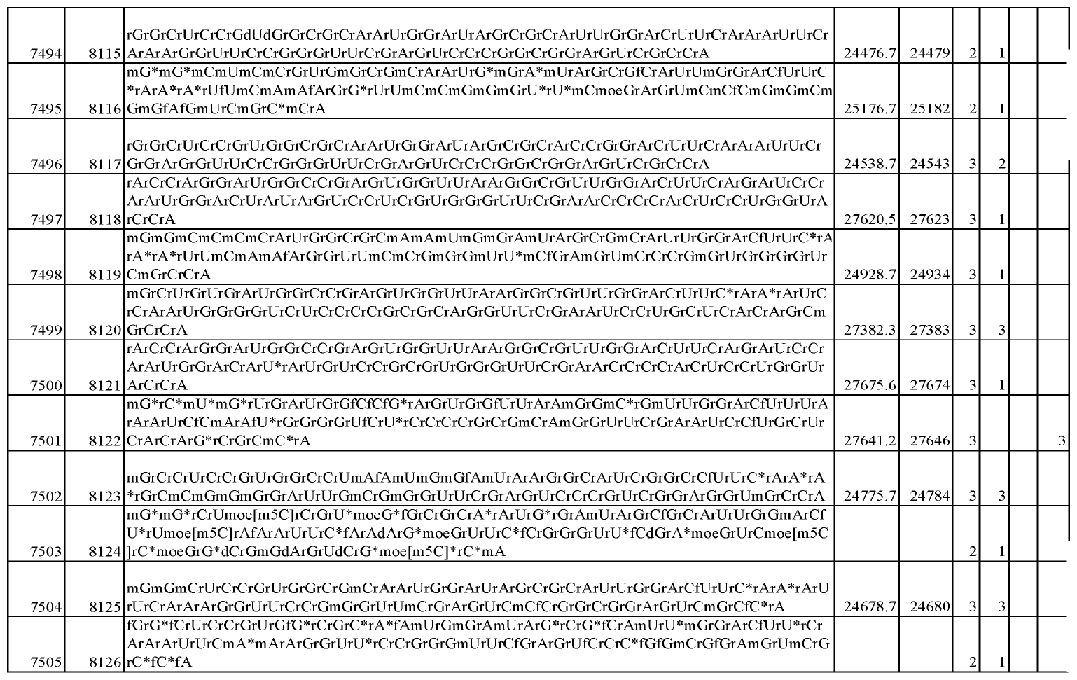

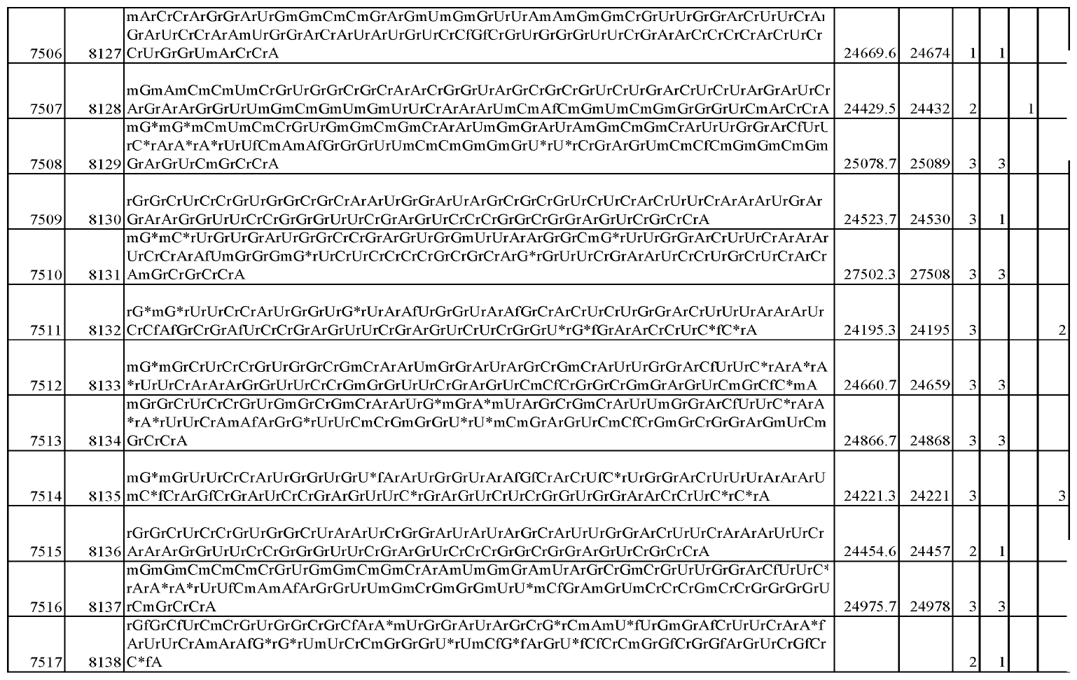

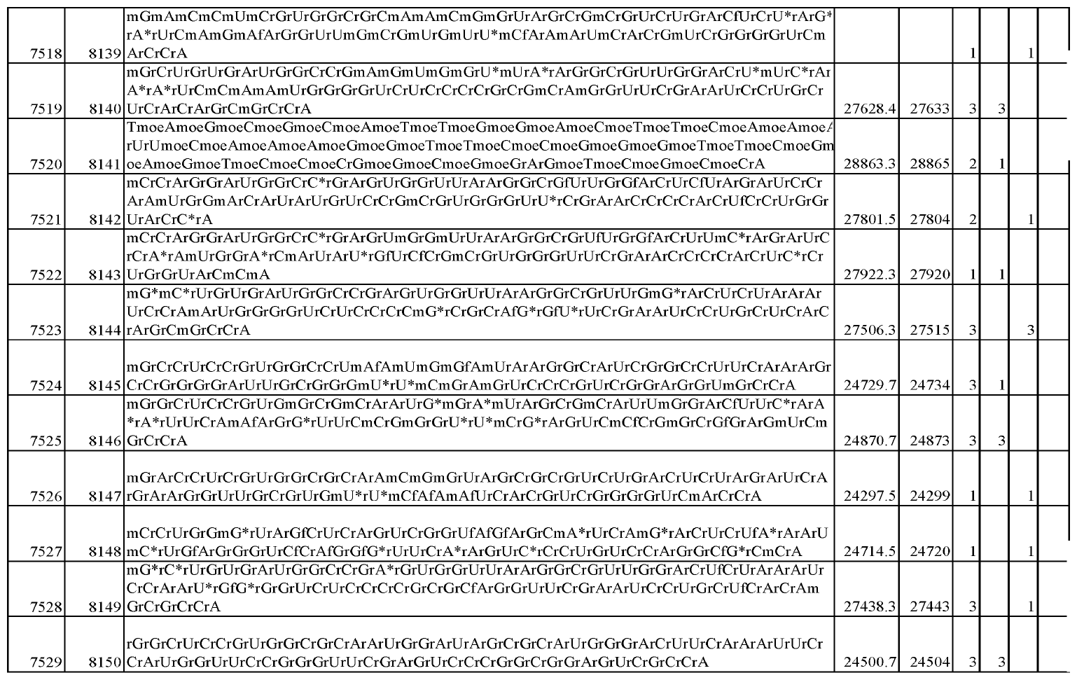

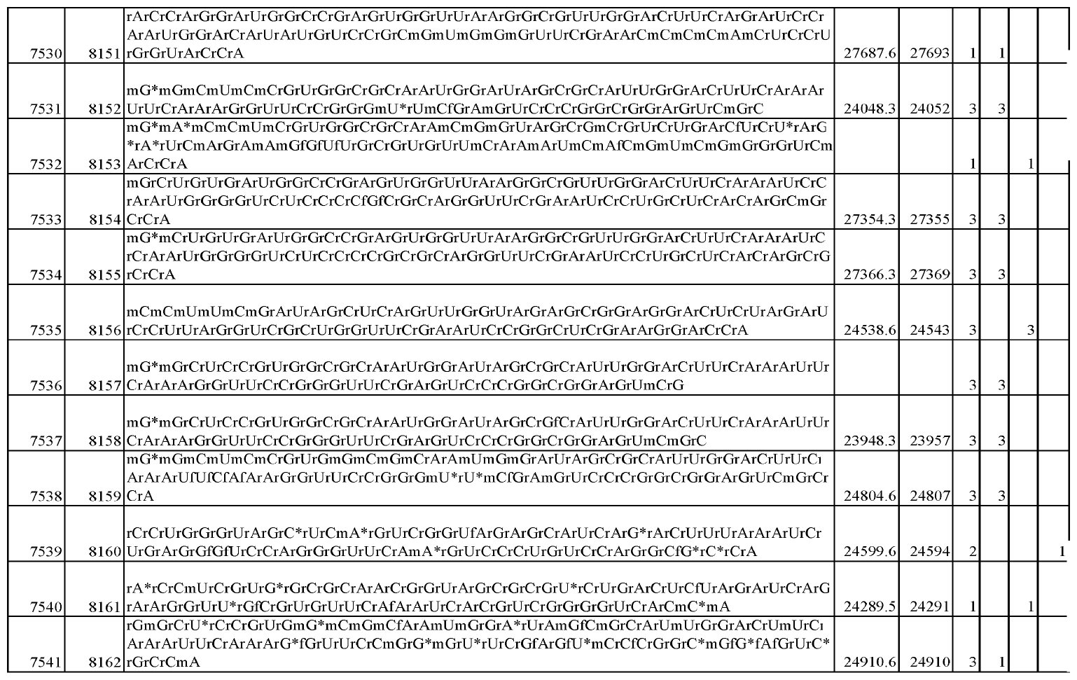

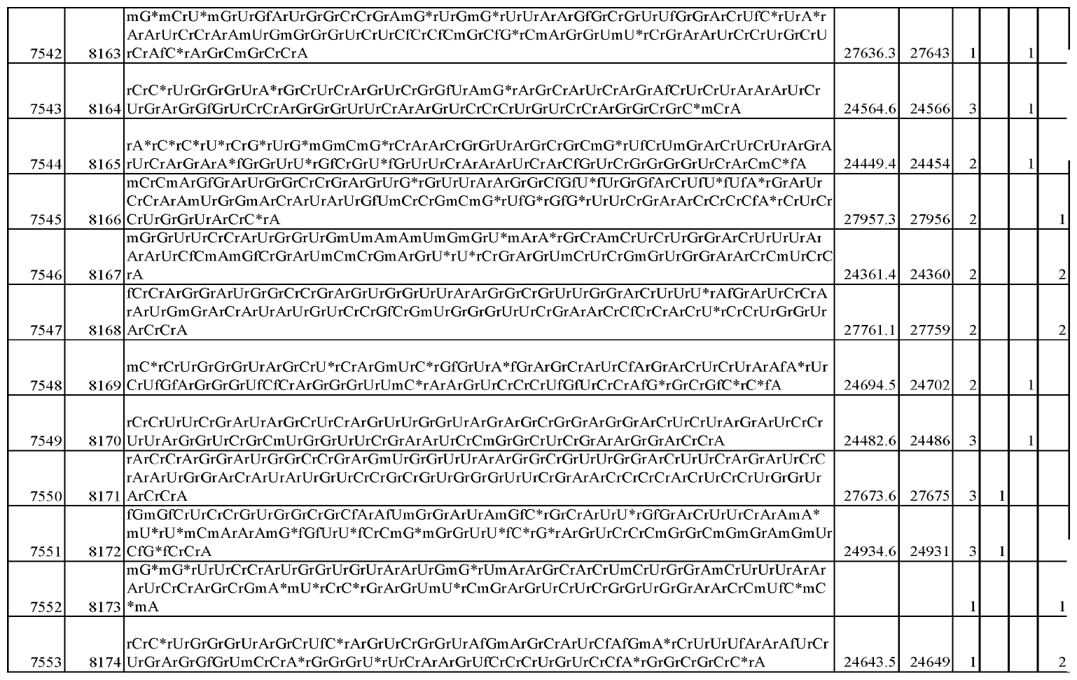

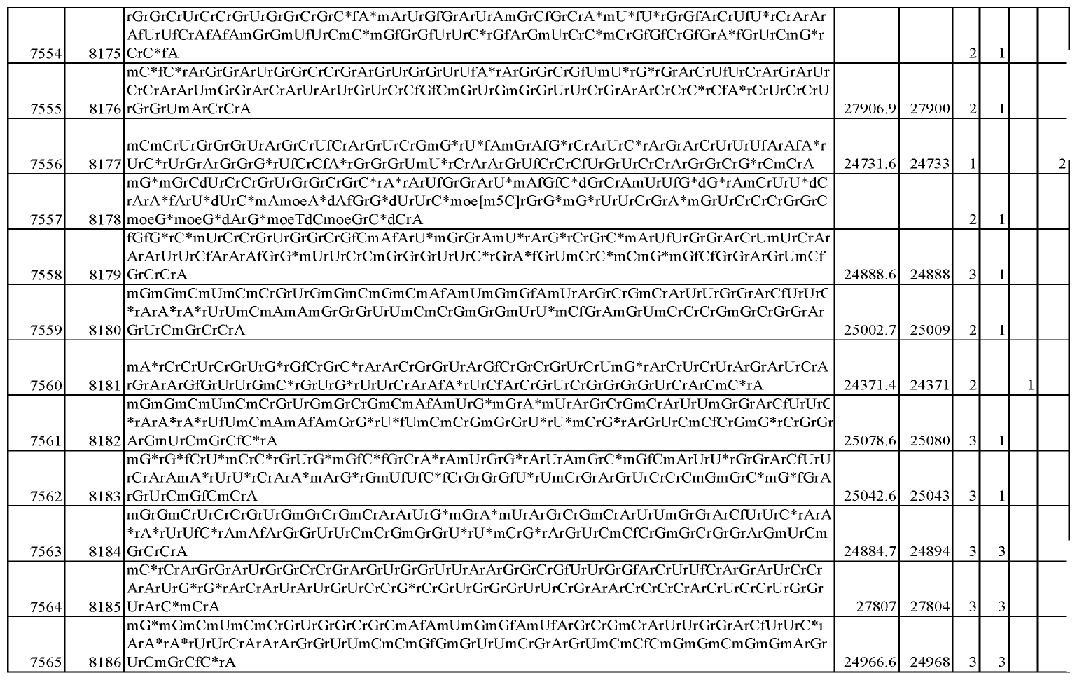

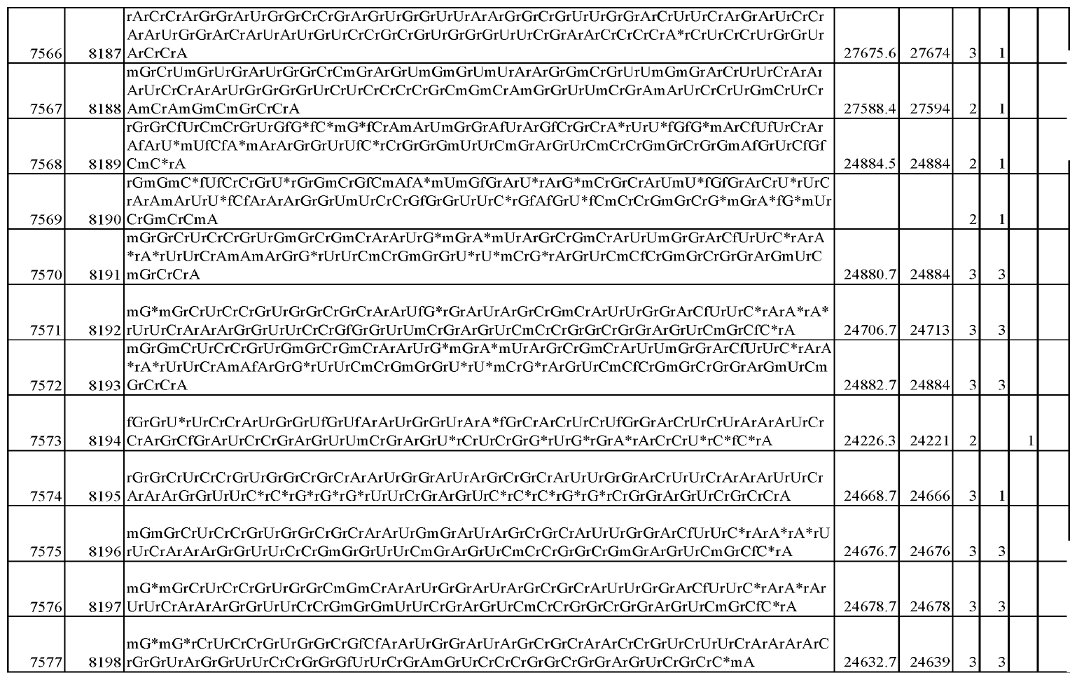

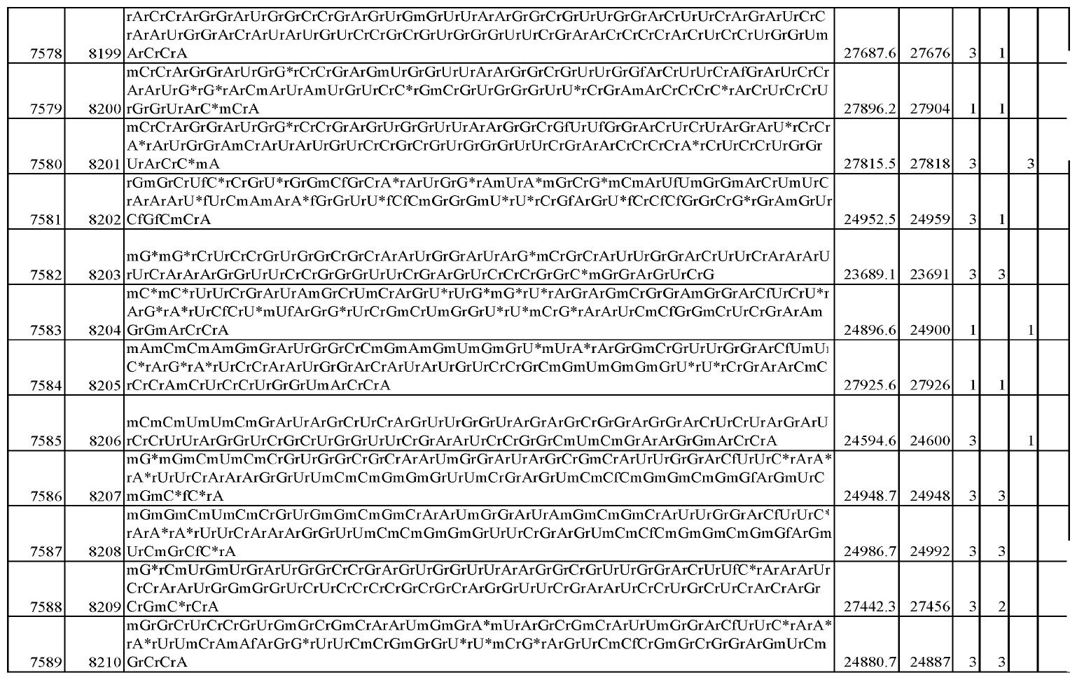

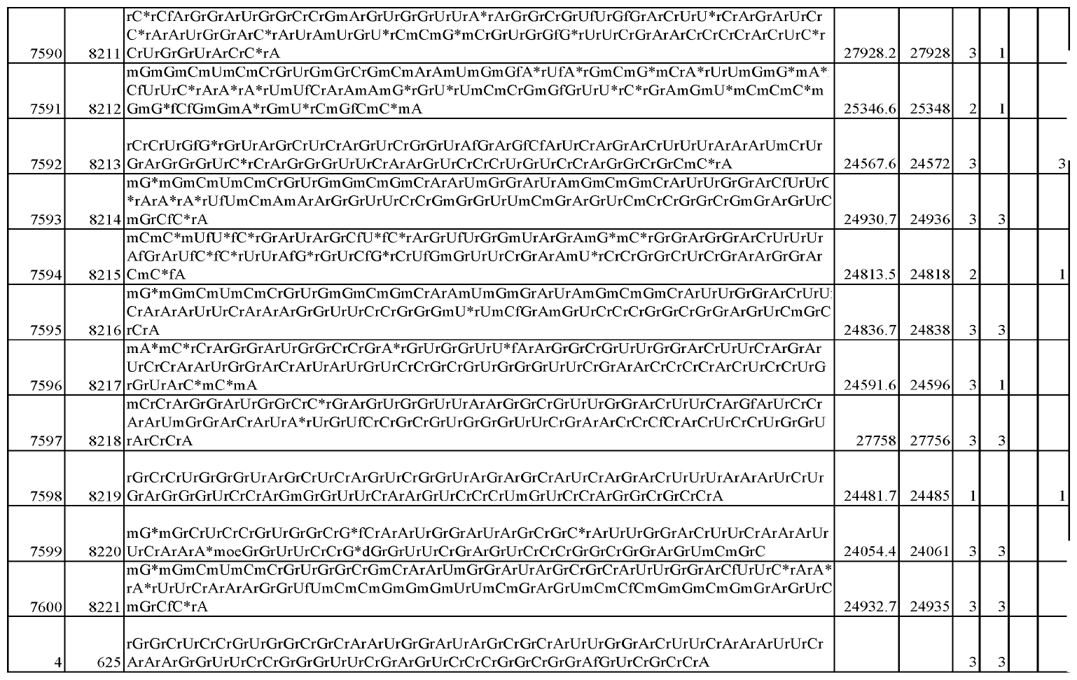

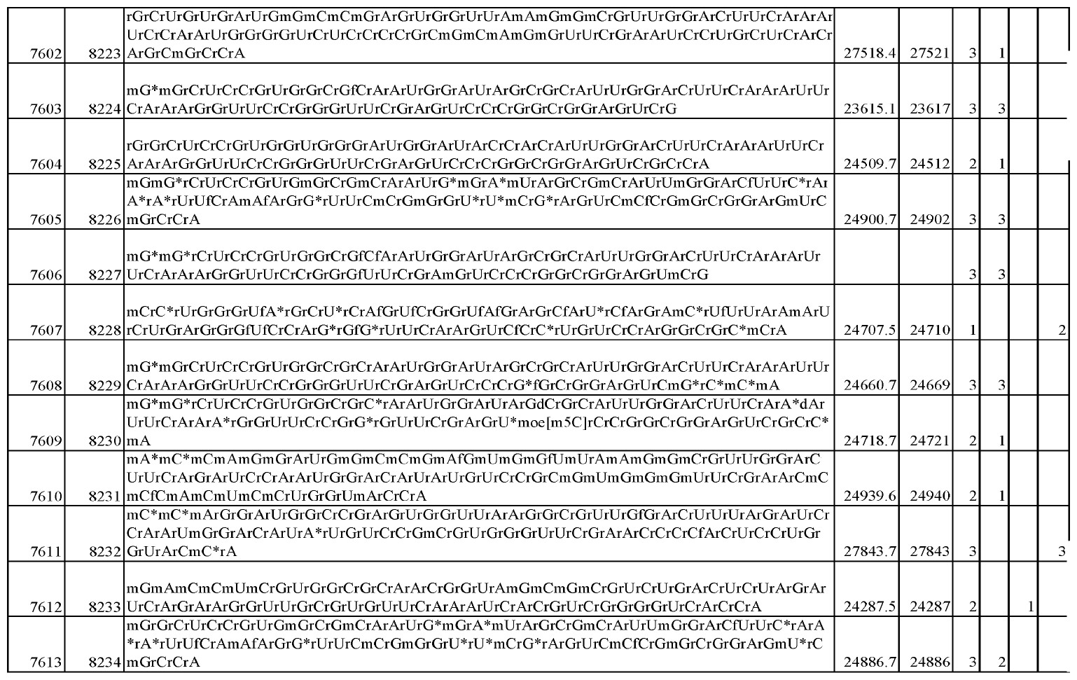

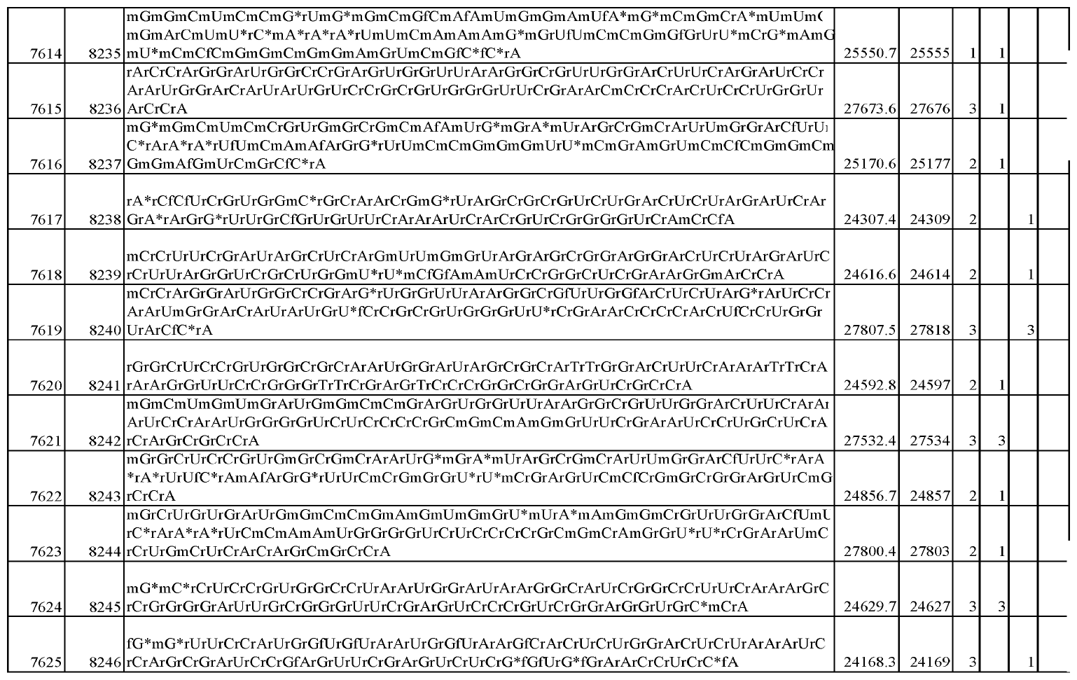

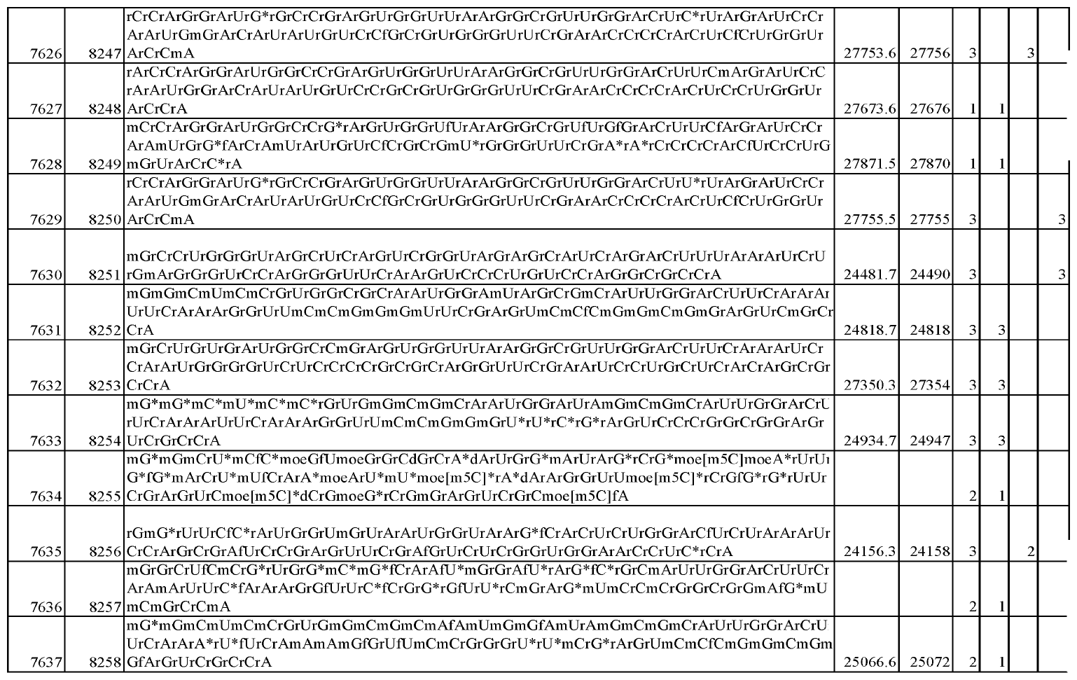

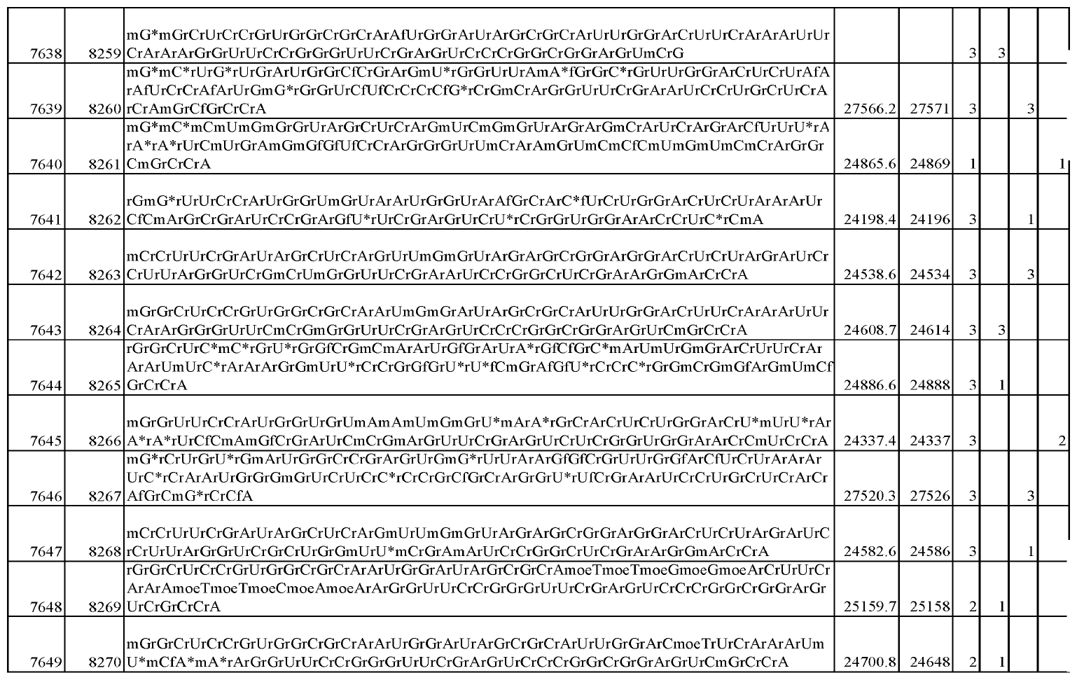

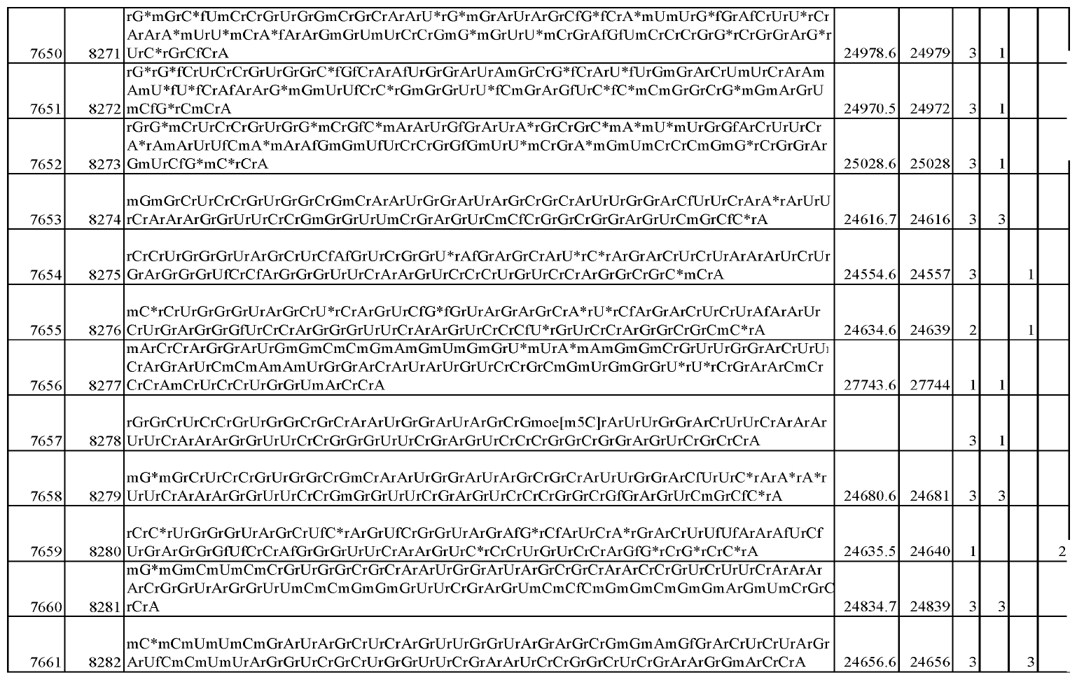

To expand on these findings, both time course and dose-response studies were carried out. In the time course study, patient fibroblasts were transiently transfected with the chemically modified TREM NO. 78 for 48 hours, 72 houts, and 96 hours, and full-length GLA protein rescue was assessed by Western blot. As shown in FIG. 5A, maximal protein rescue (approximately 50% versus normal healthy fibroblasts) across all time points was observed 96 hours post-transfection. In the dose-response study, TREM NO. 78 was provided to cells at doses of 2.6 nM, 16 nM, 40 nM, and 100 nM, and cells were harvested 96 hours post-transfection. As shown in FIG. 5B & FIG. 5D, full-length GLA protein rescue levels rise until reaching a plateau at 40 nM. In addition, GLA transcript levels also increased in a concentration-dependent fashion as measured by qPCR, suggesting that TREM NO. 78 also rescues GLA mRNA nonsense mediated decay (NMD) in Fabry patient fibroblasts as shown in FIG. 5E.

Additional studies were carried out to assess whether administration of exemplary TREMs was sufficient to rescue GLA enzymatic activity. Fabry patient fibroblasts were treated with either TREM NO. 2309 or TREM NO. 78 at a dose of either 20 nM or 40 nM, and GLA activity was measured 96 hours post-transfection. An established GLA enzymatic activity was performed on cell extracts using a GLA-specific substrate that results in the generation of a fluorescent cleavage product (4-methylumbelliferone). As shown in FIG. 6, GLA activity increased following treatment with both TREMs, though administration of chemically modified TREM NO. 78 resulted in a profound rescue of GLA activity greater than or equal to GLA activity levels in normal healthy fibroblasts at both doses.

### Example 12: In Vivo PTC Readthrough and Target Engagement of TREM by Hydrodynamic Gene Delivery

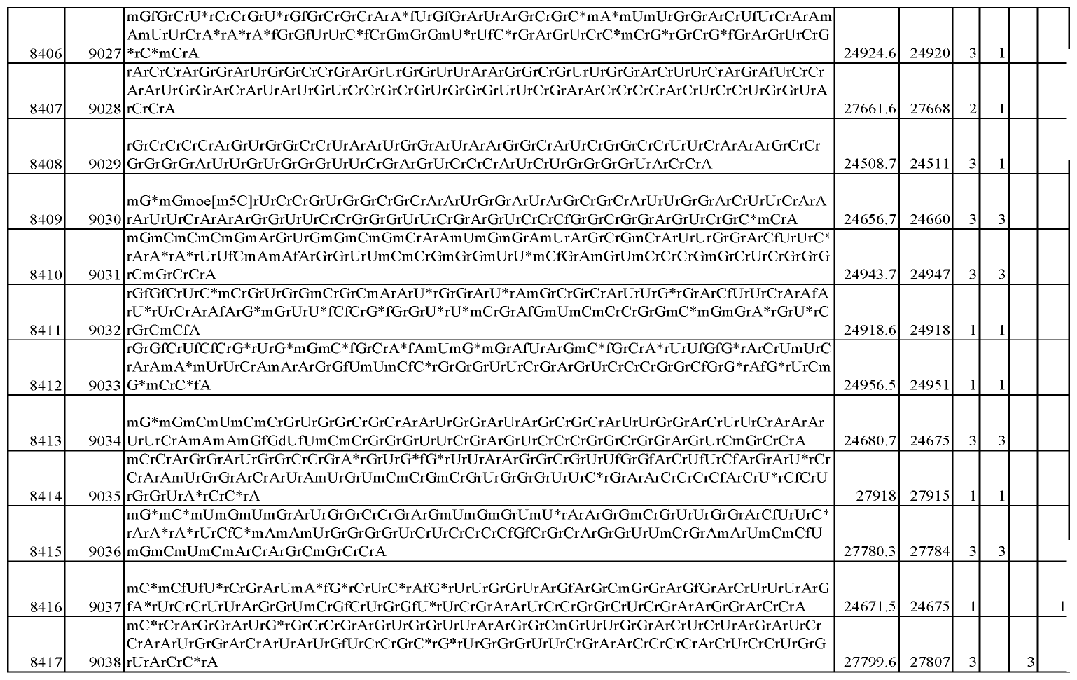

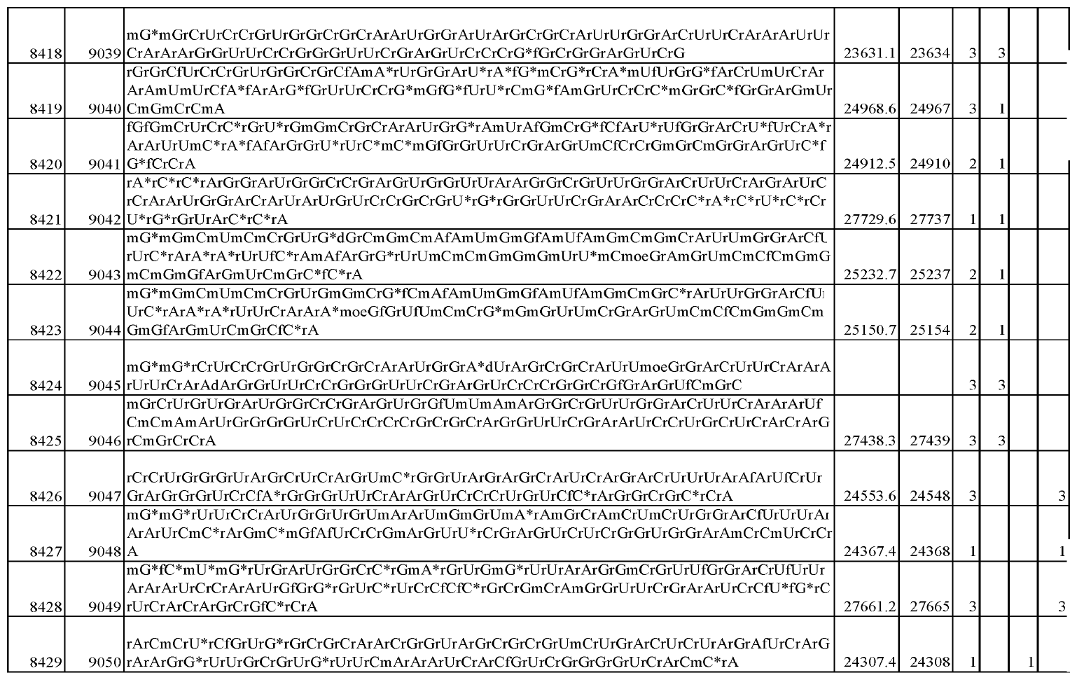

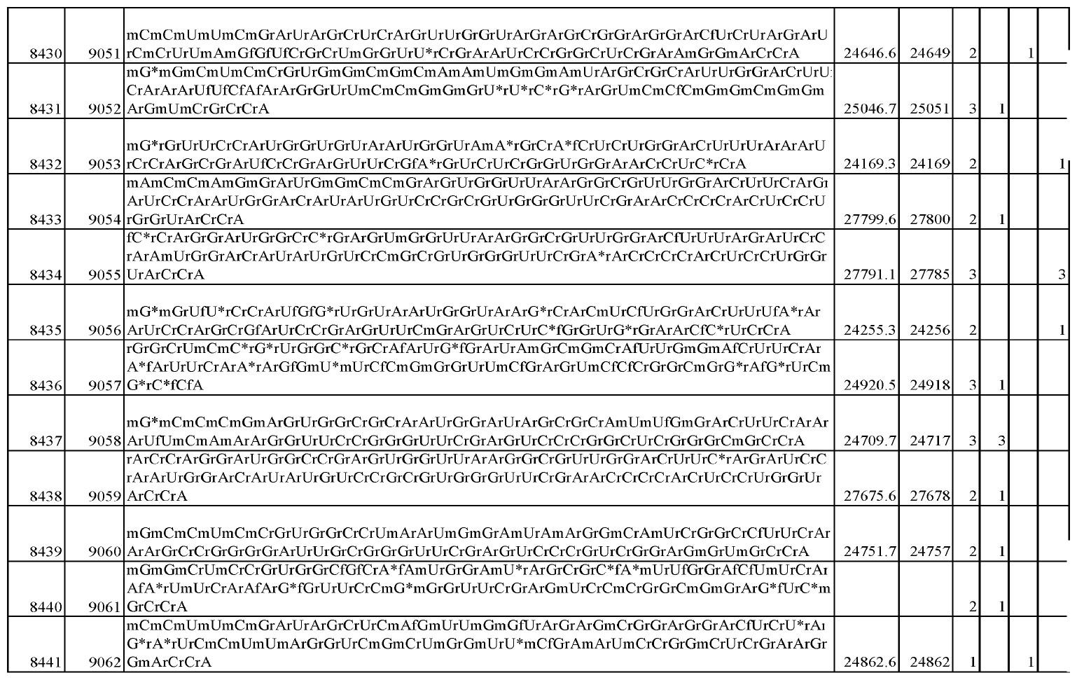

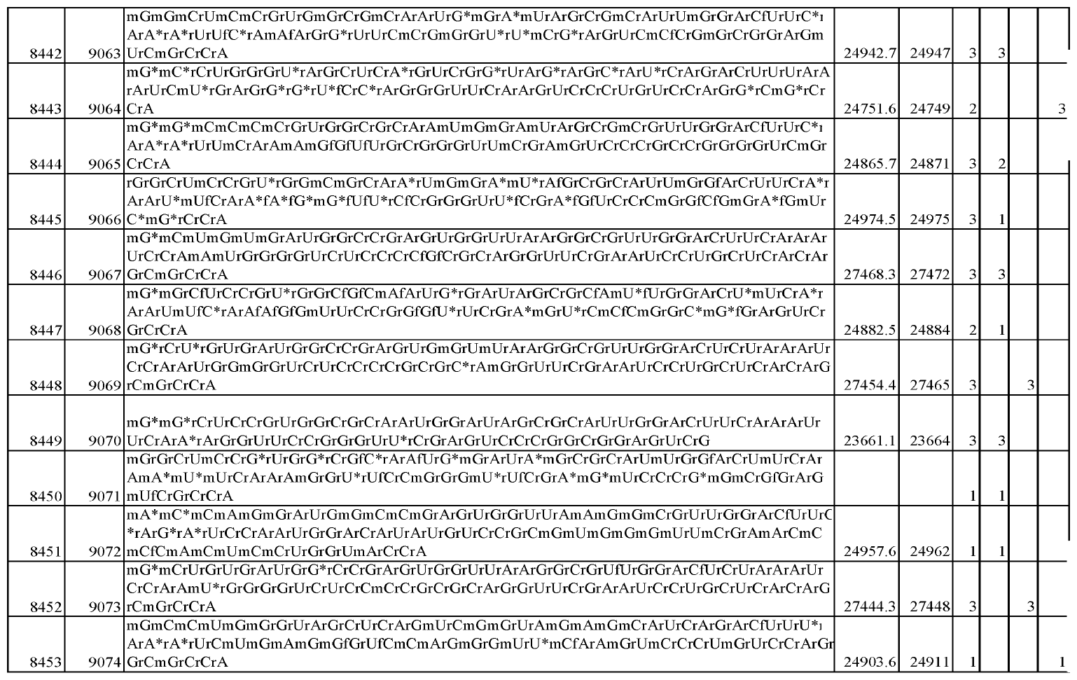

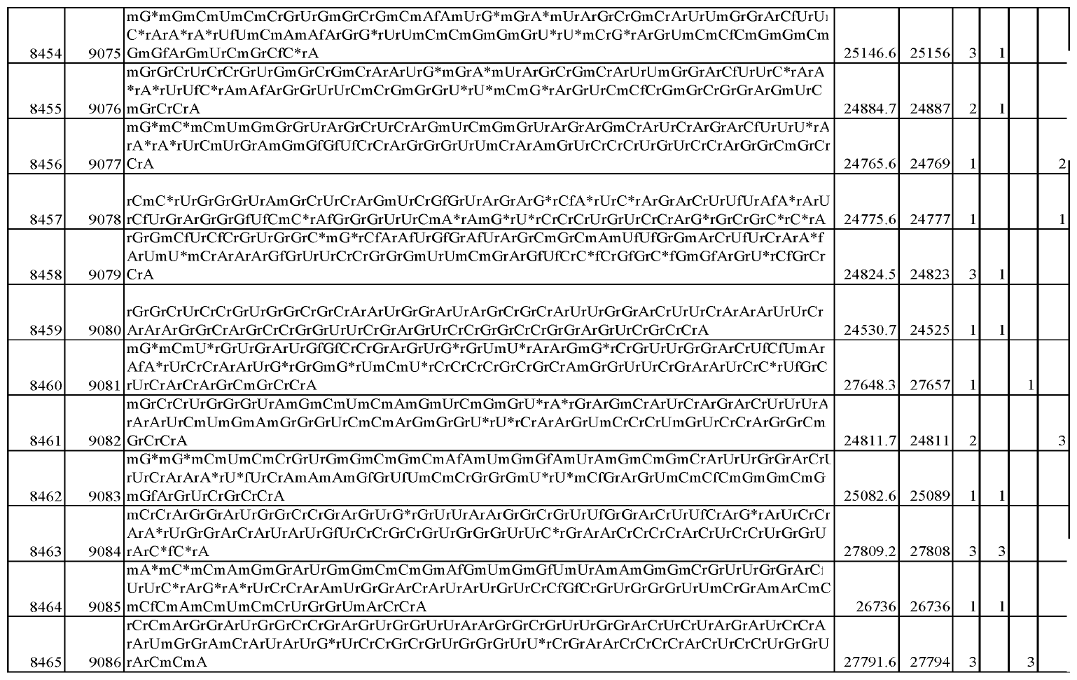

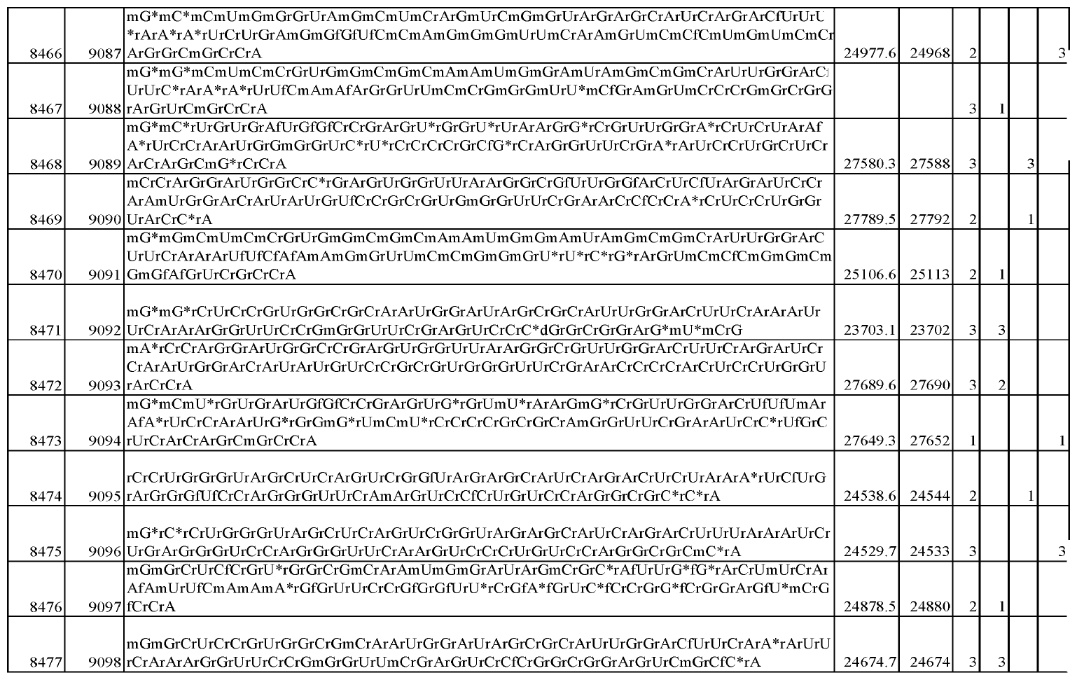

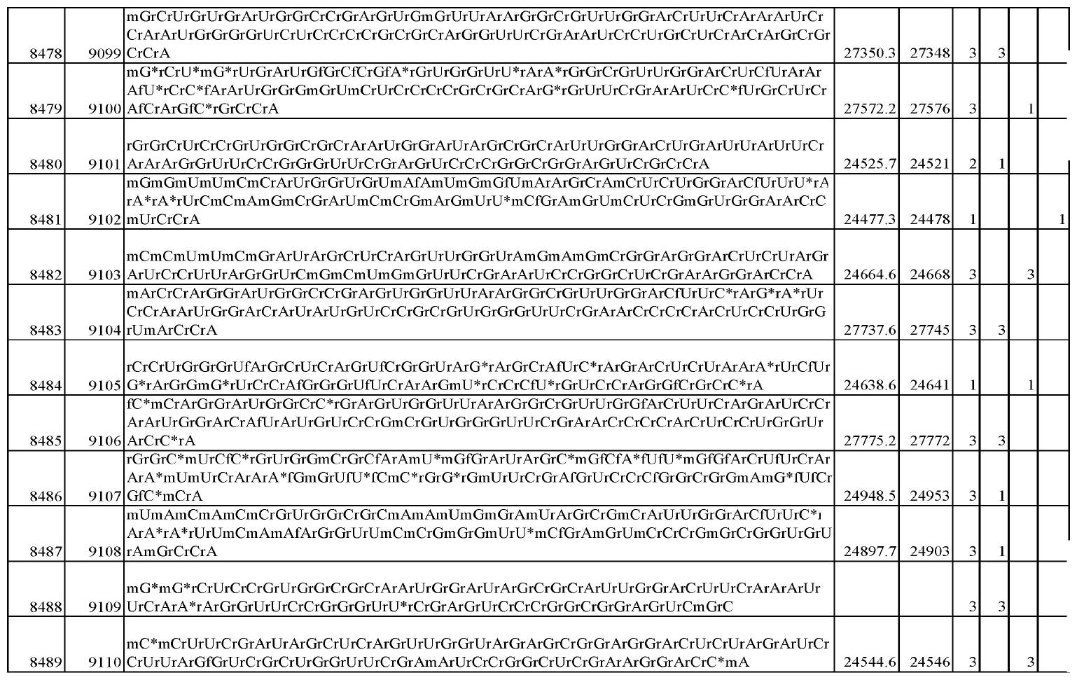

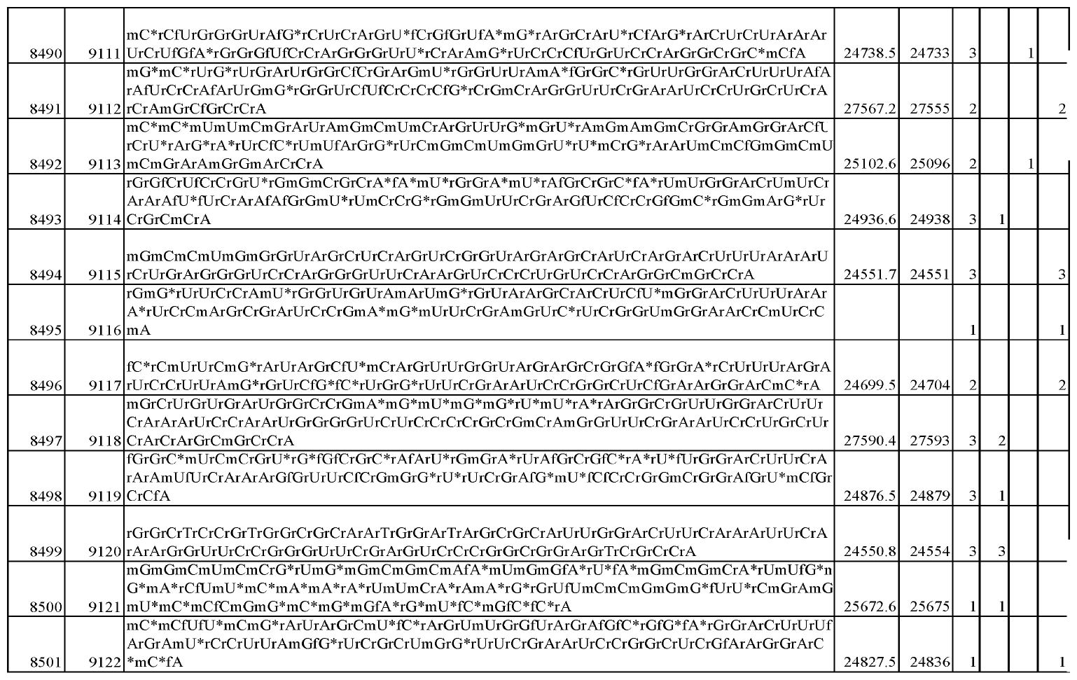

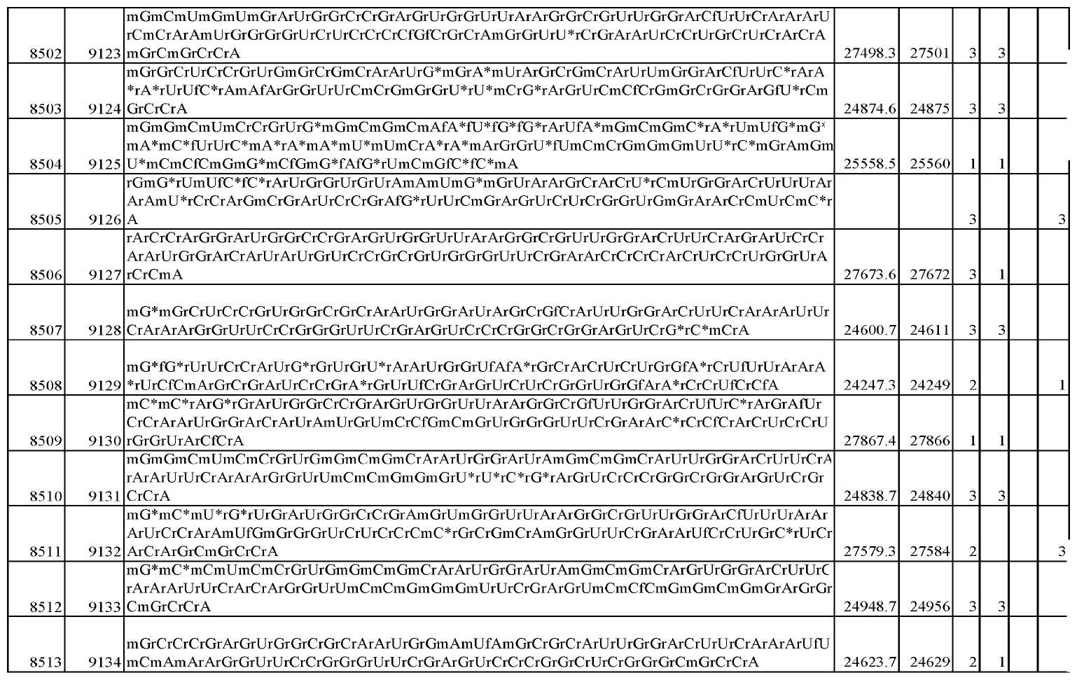

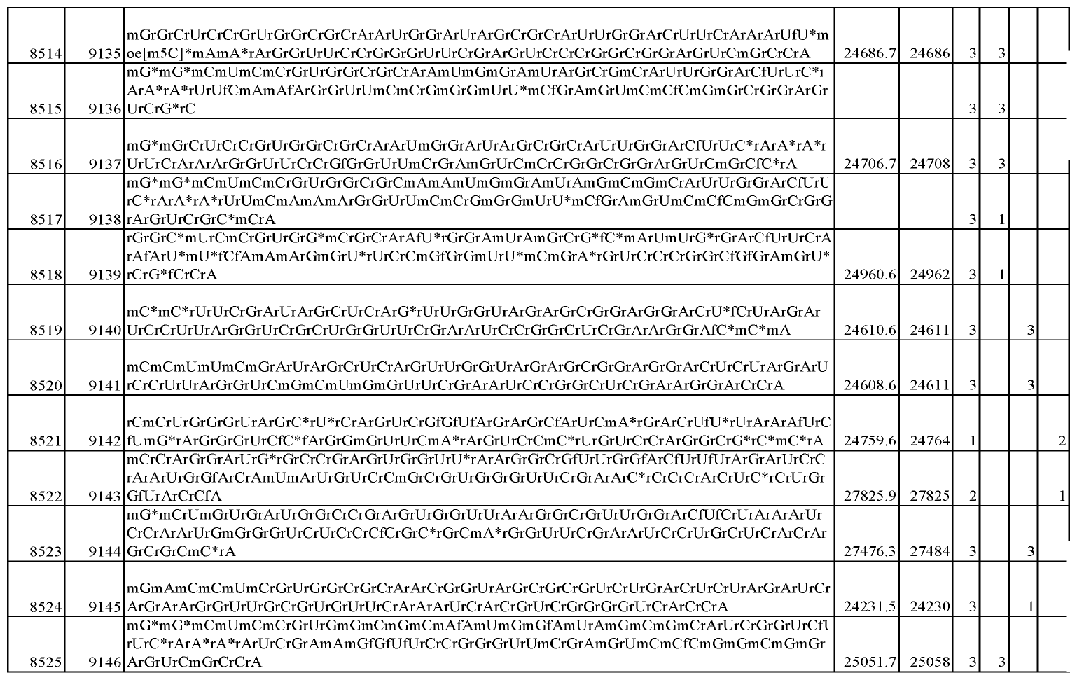

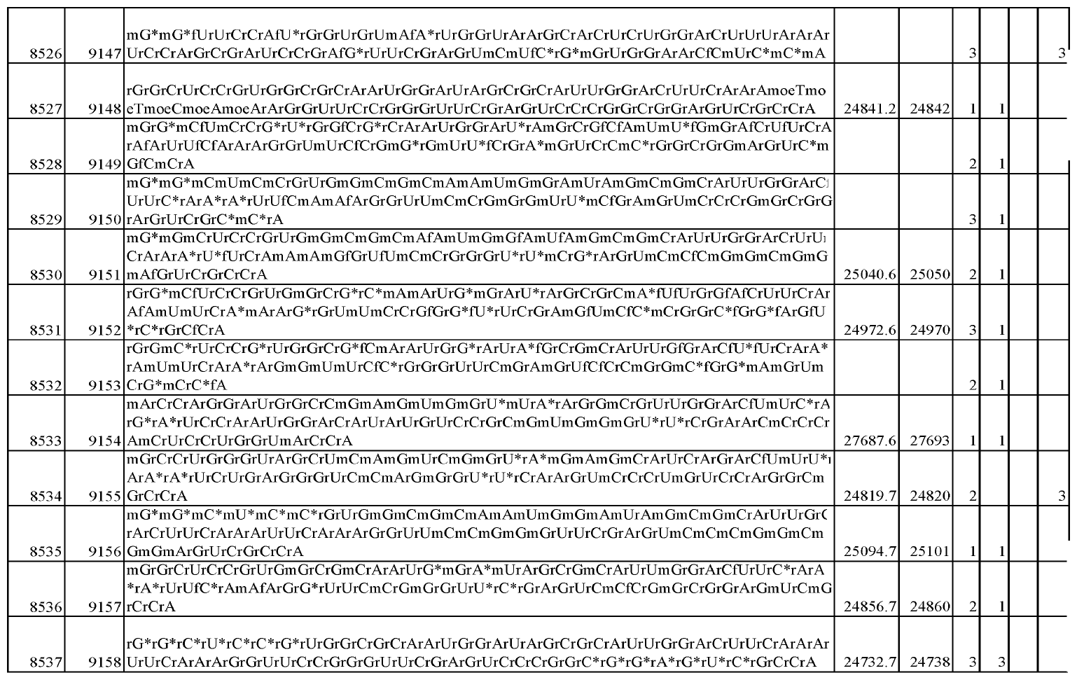

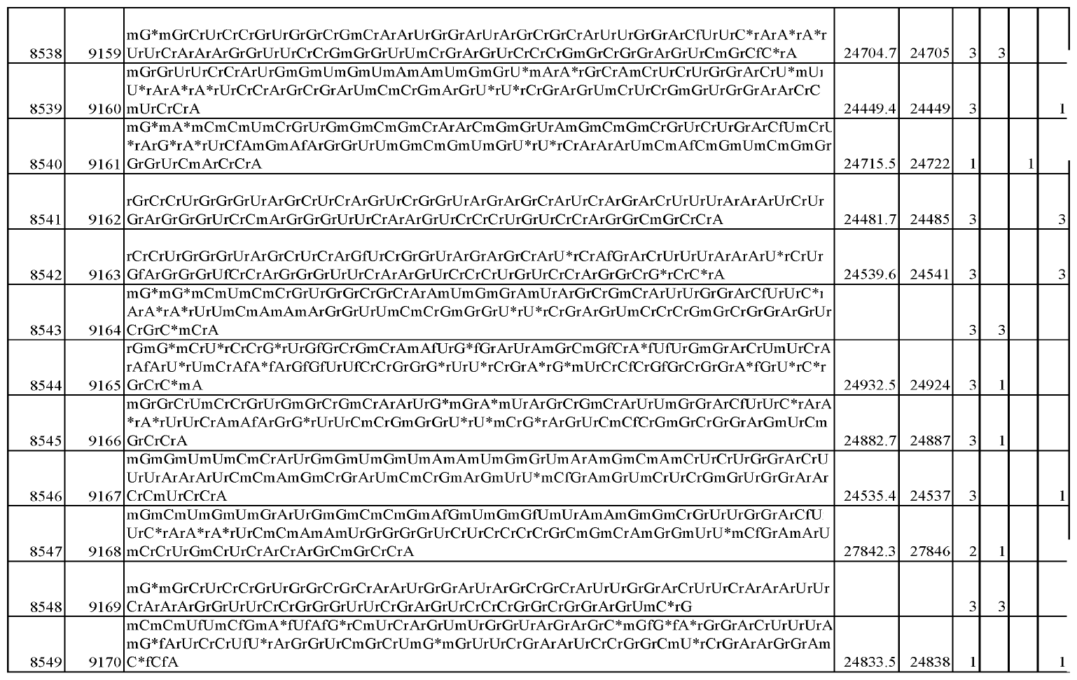

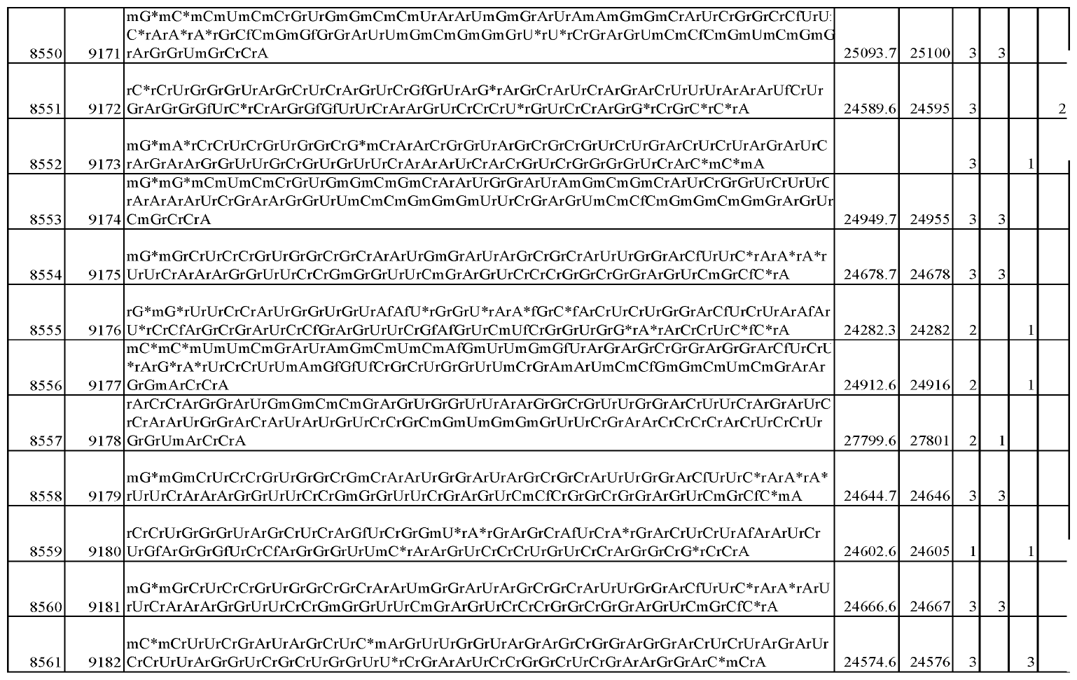

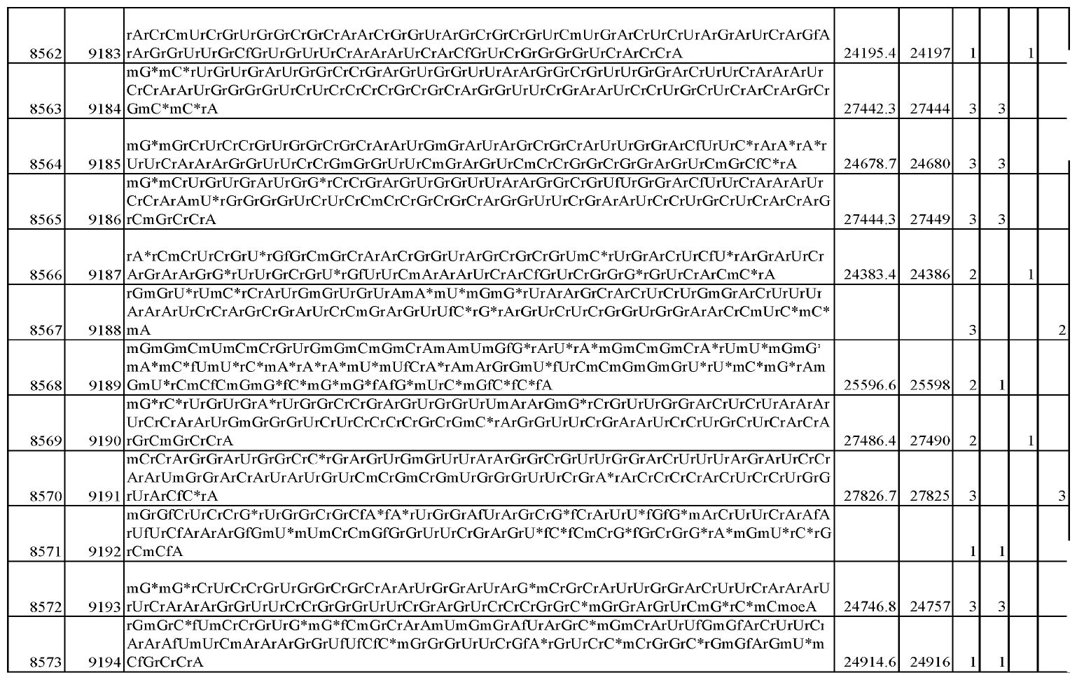

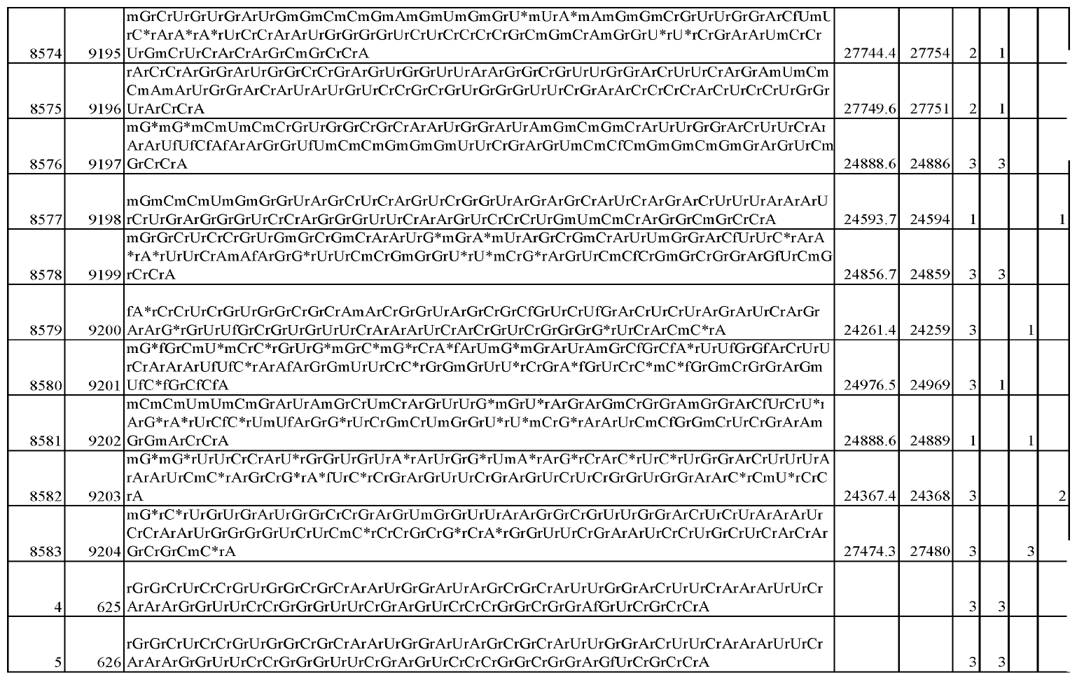

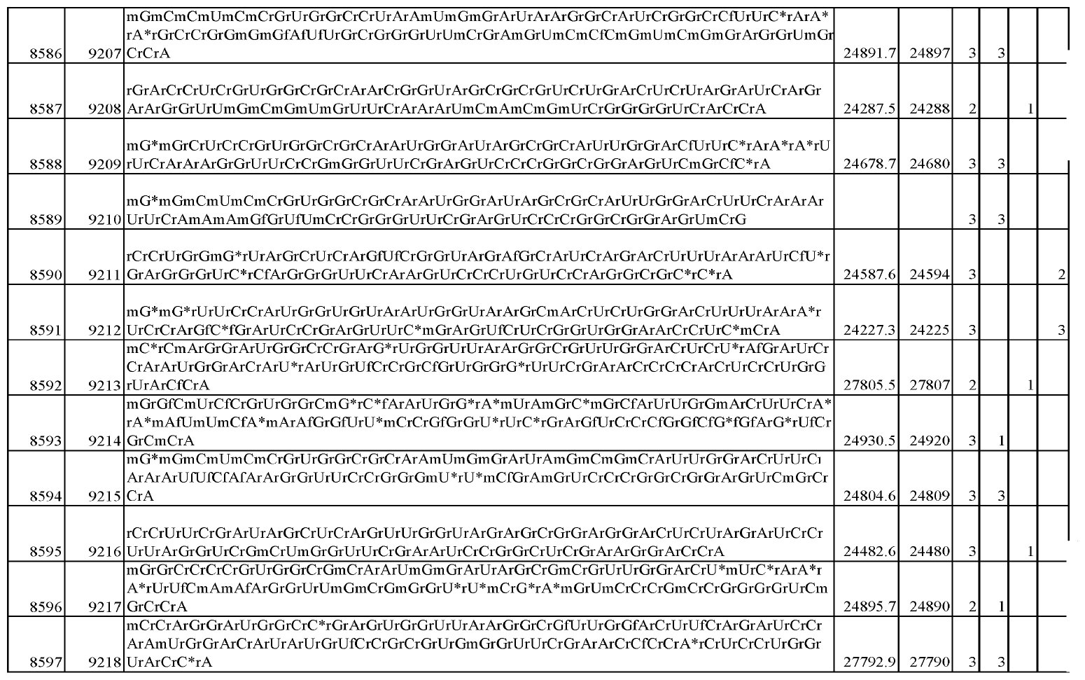

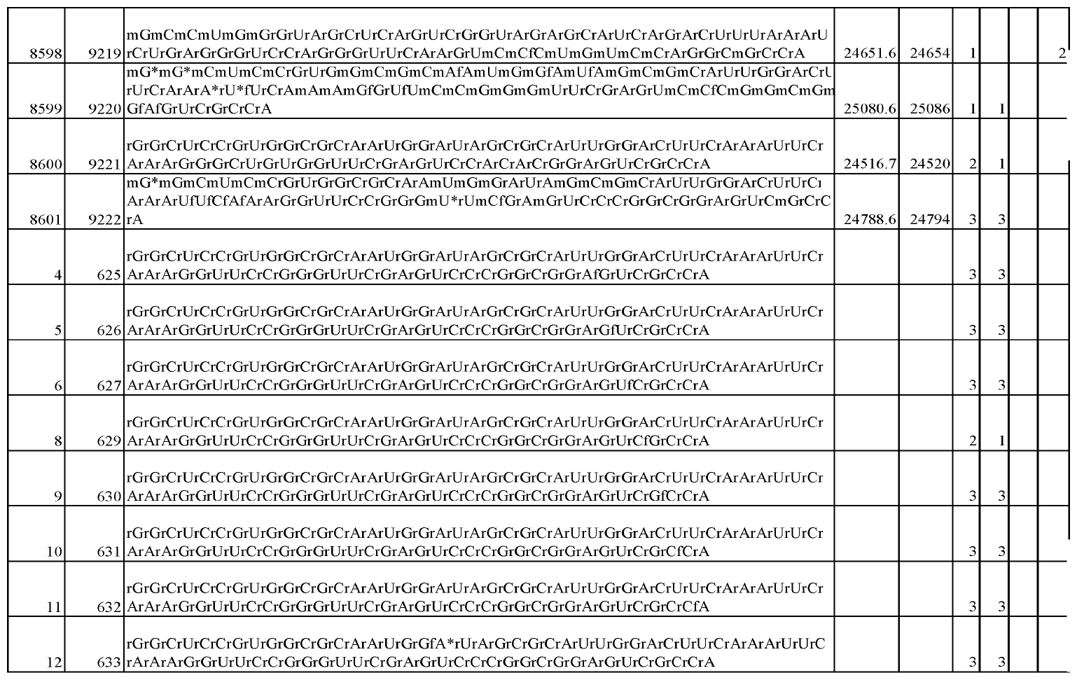

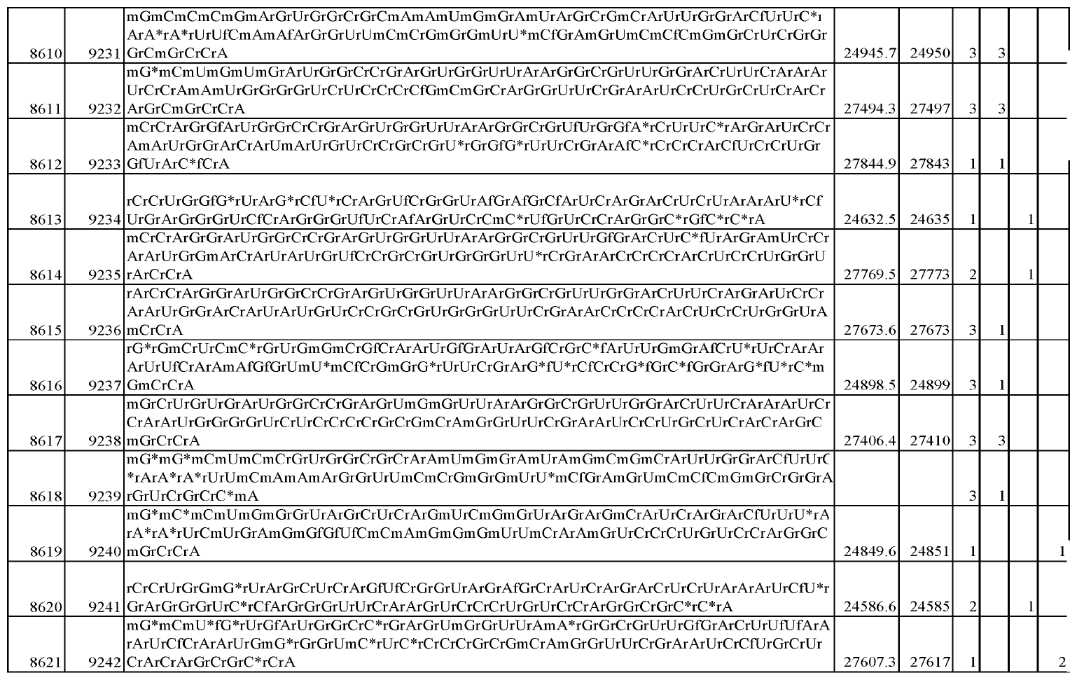

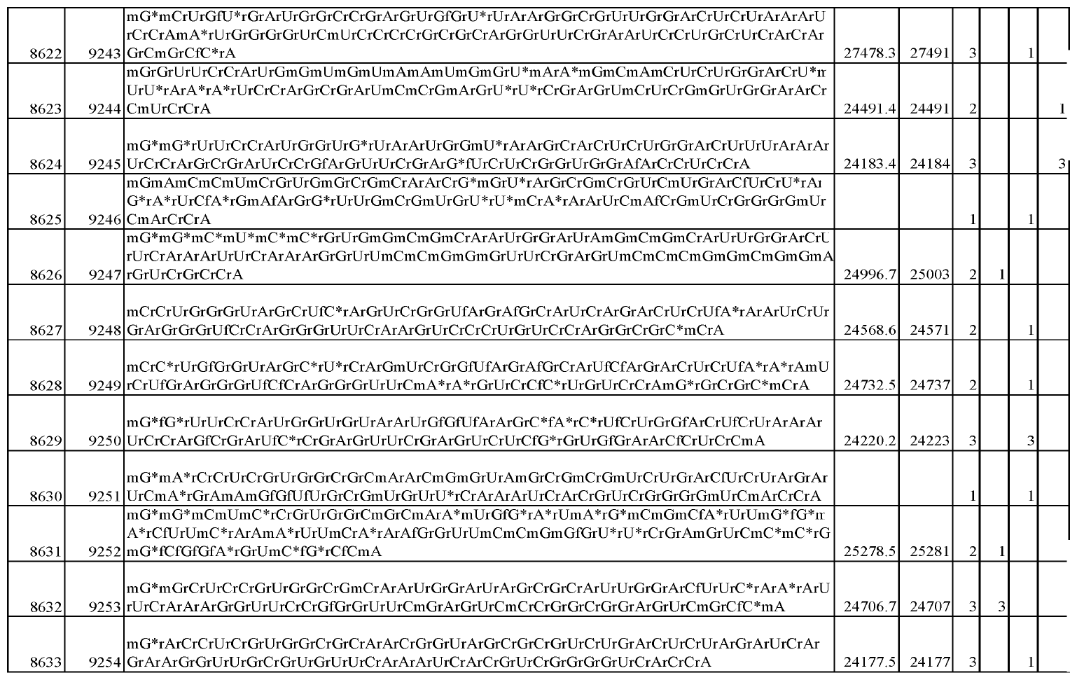

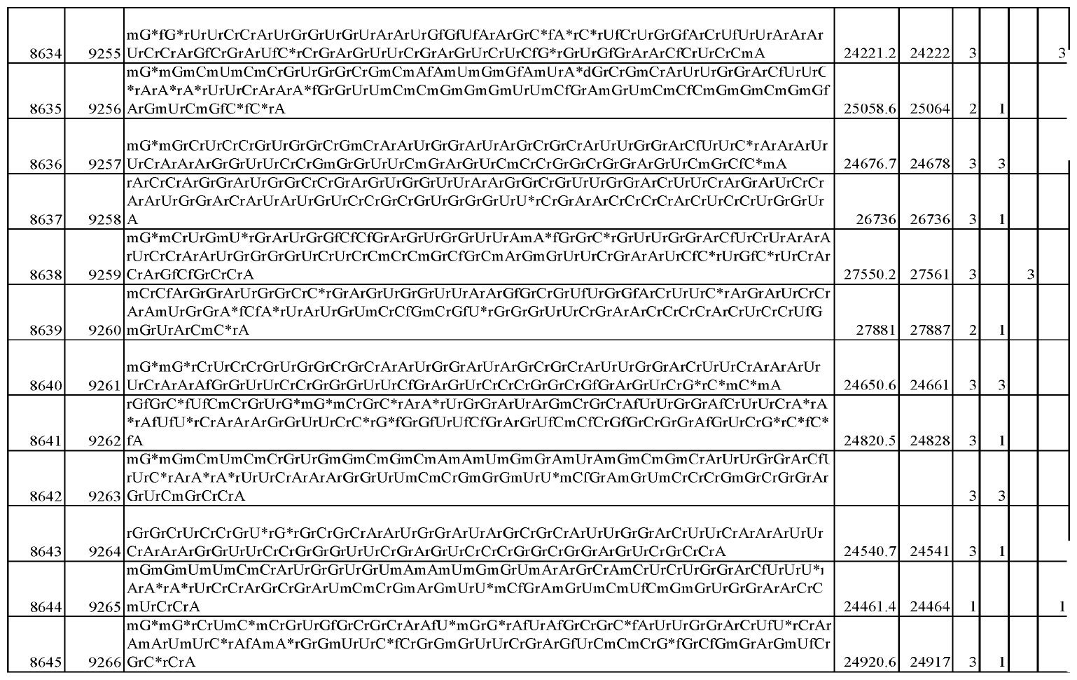

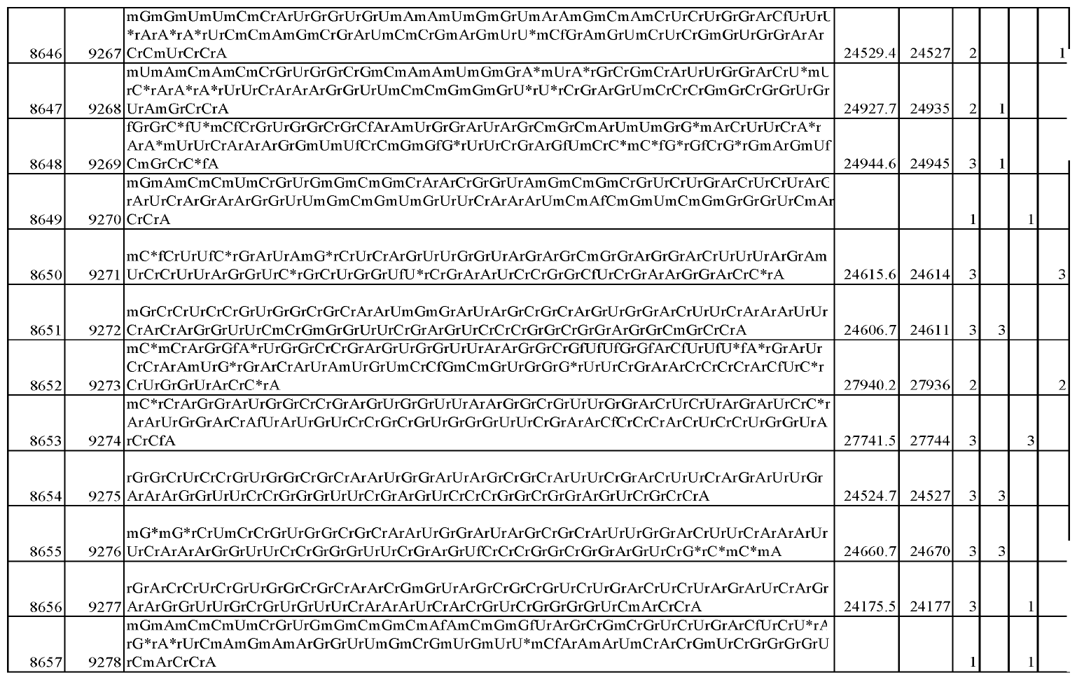

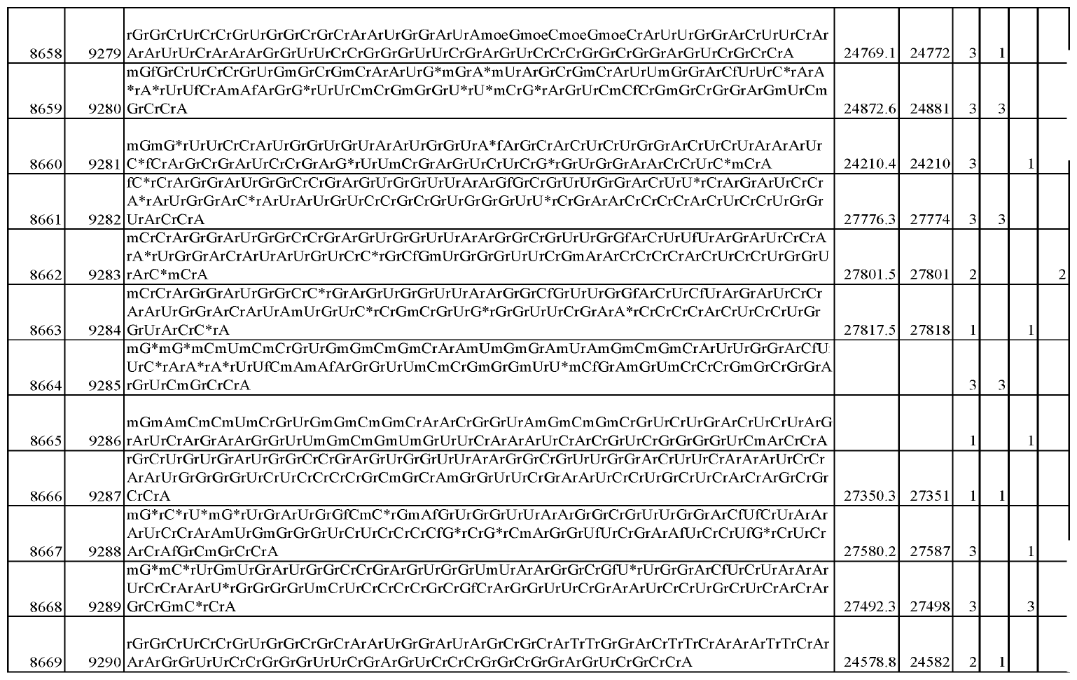

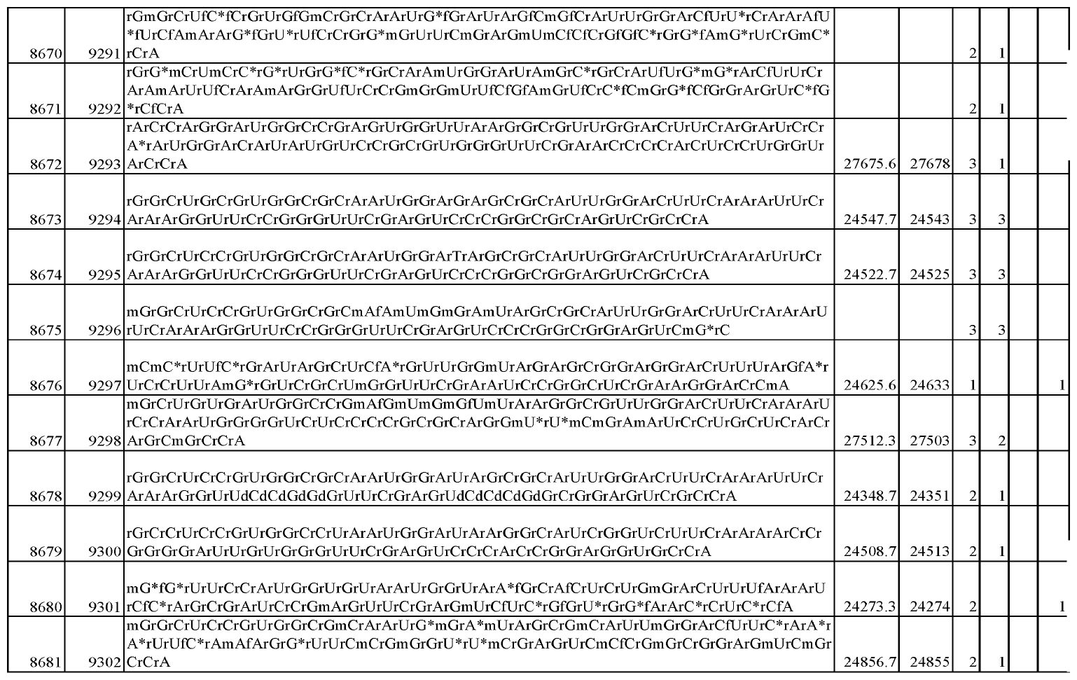

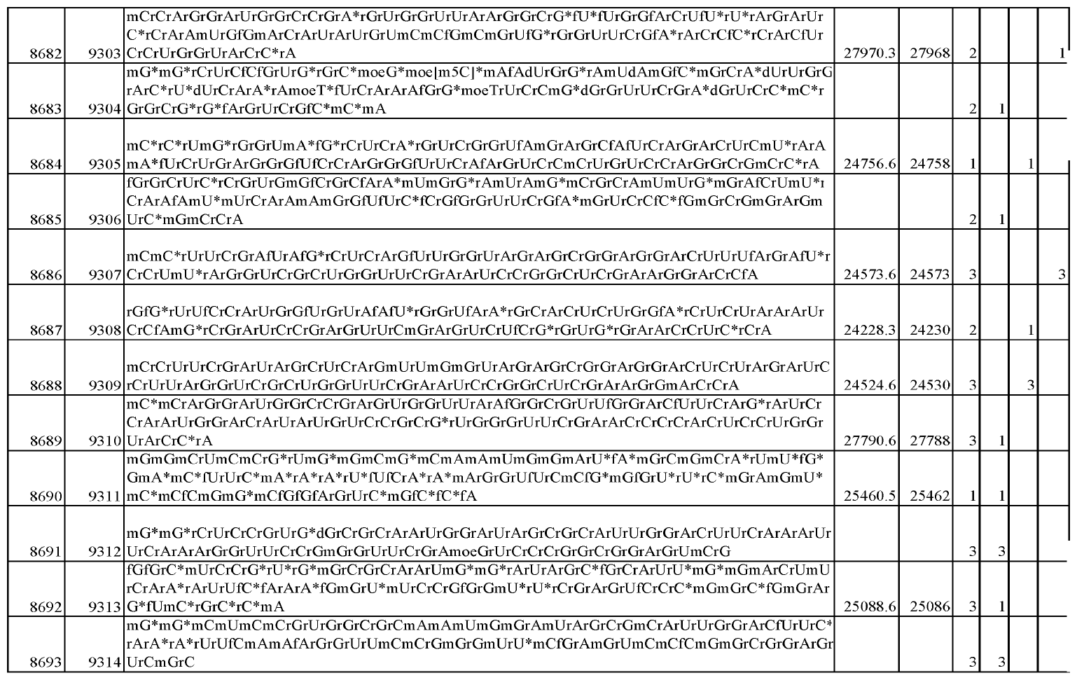

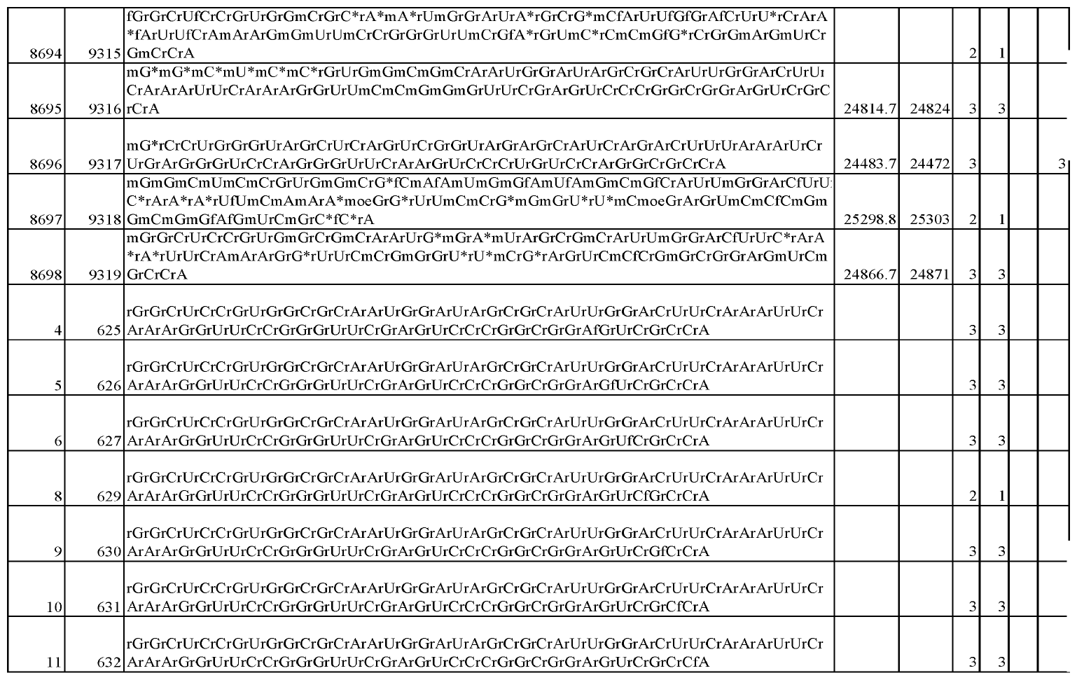

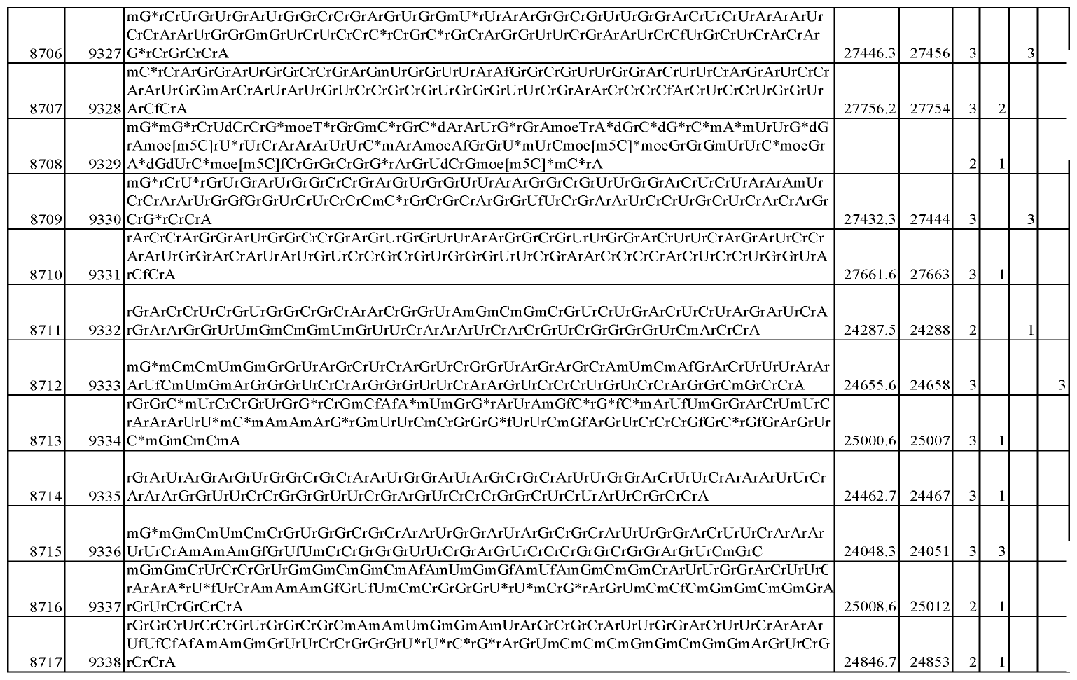

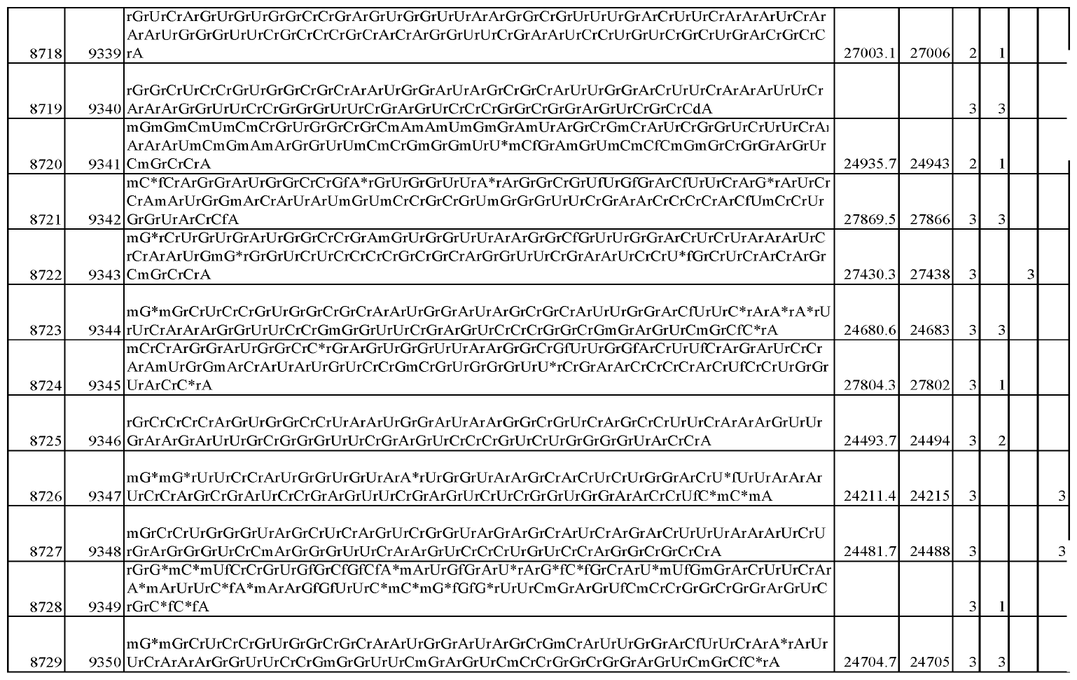

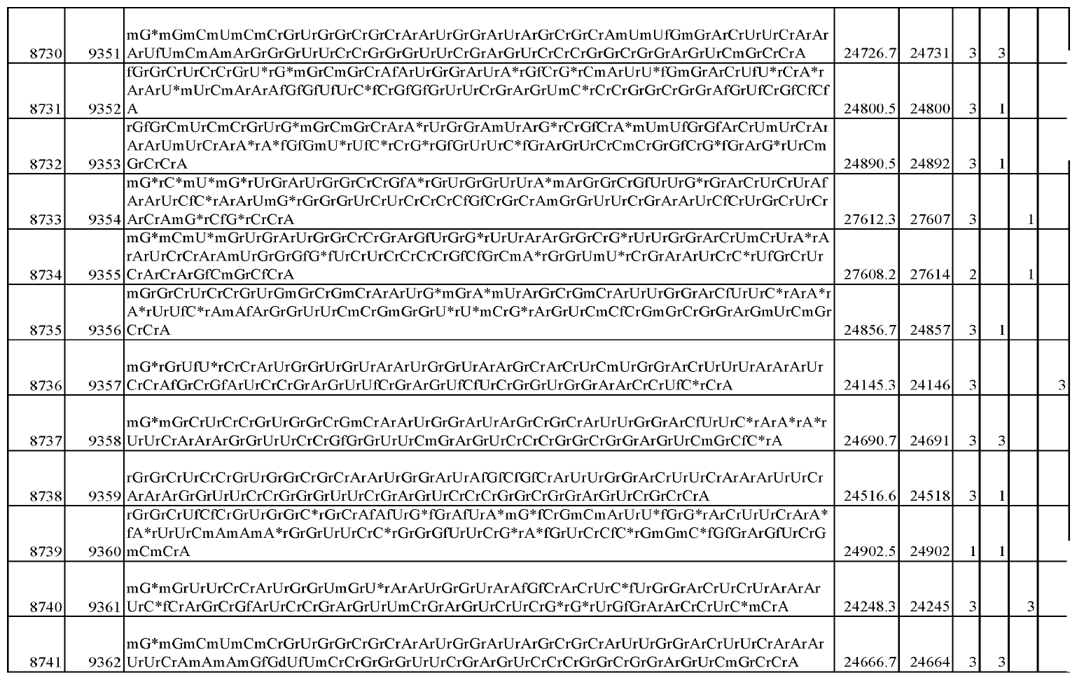

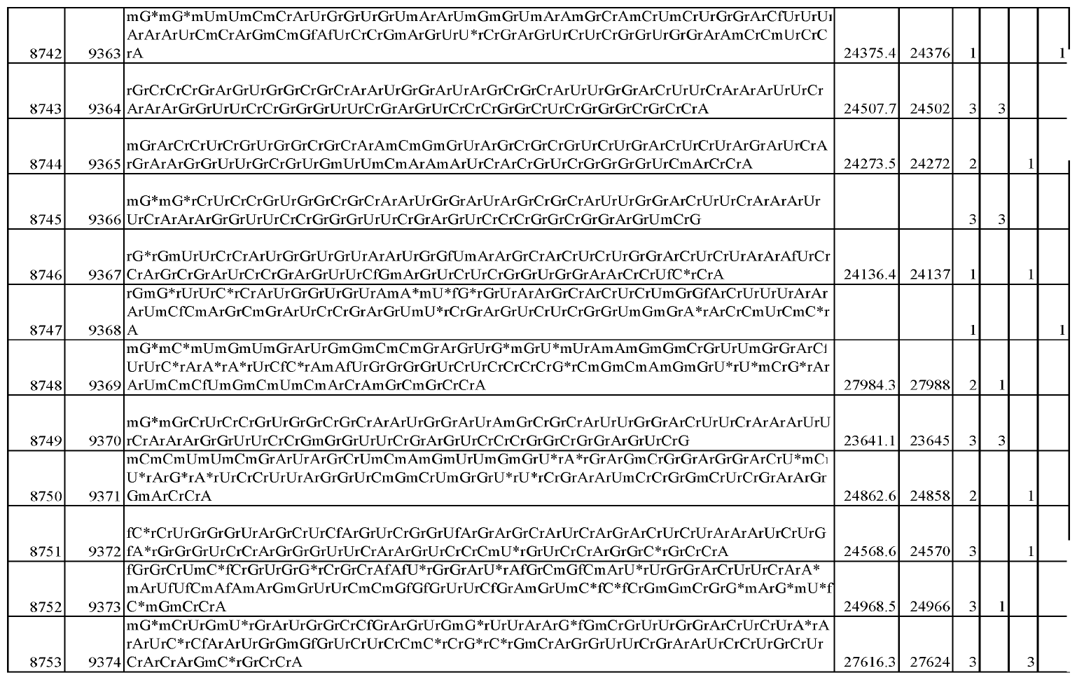

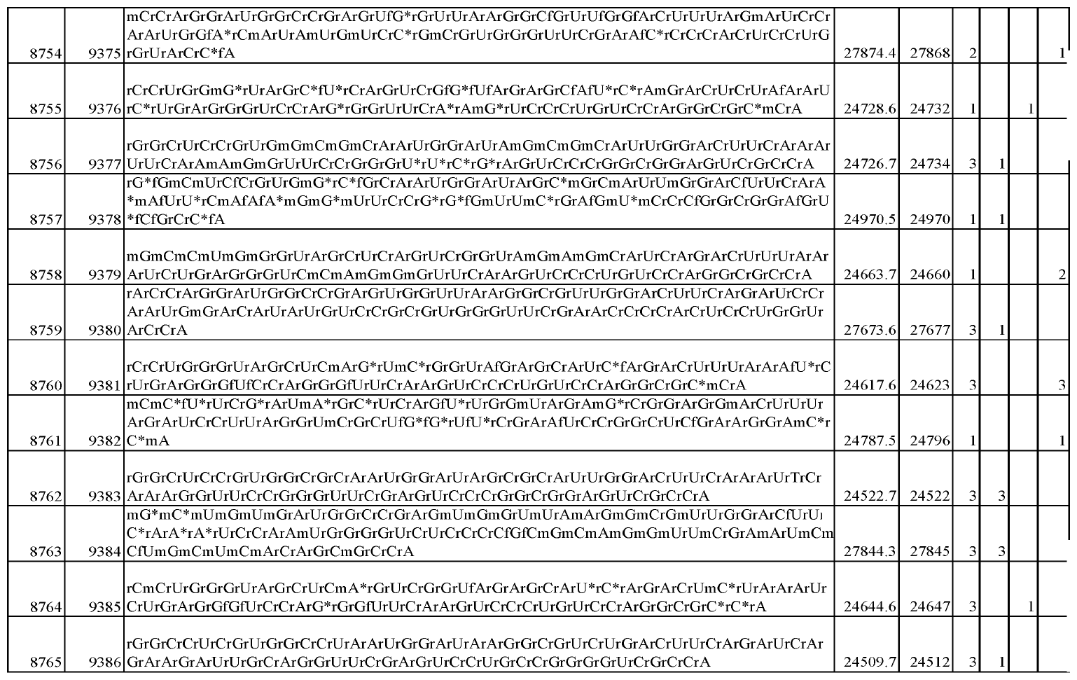

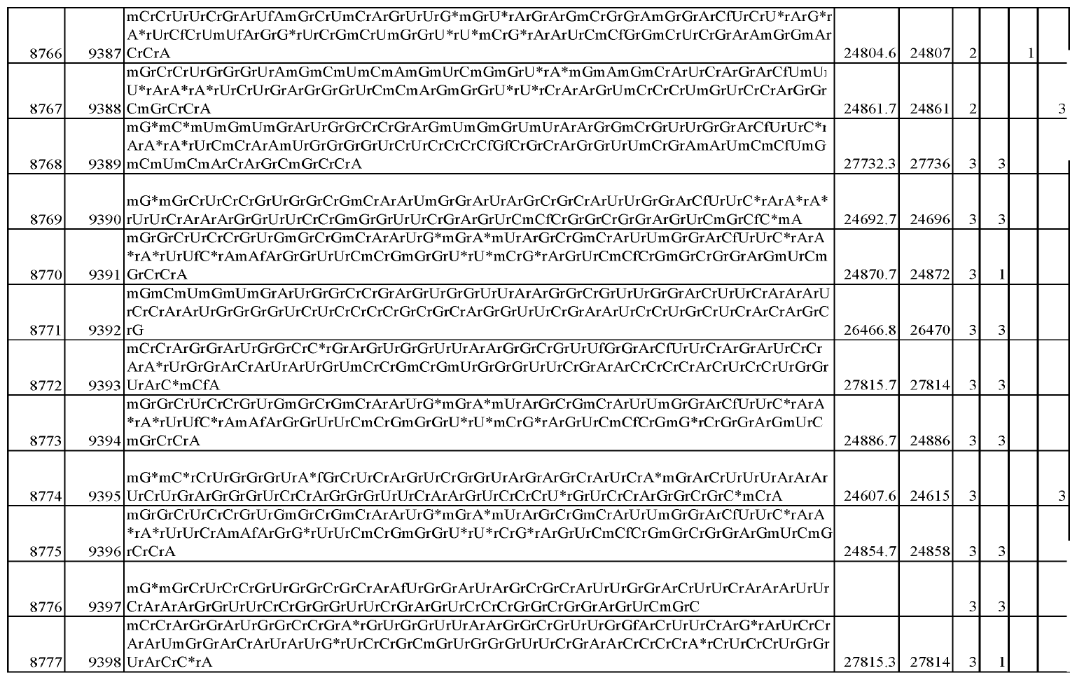

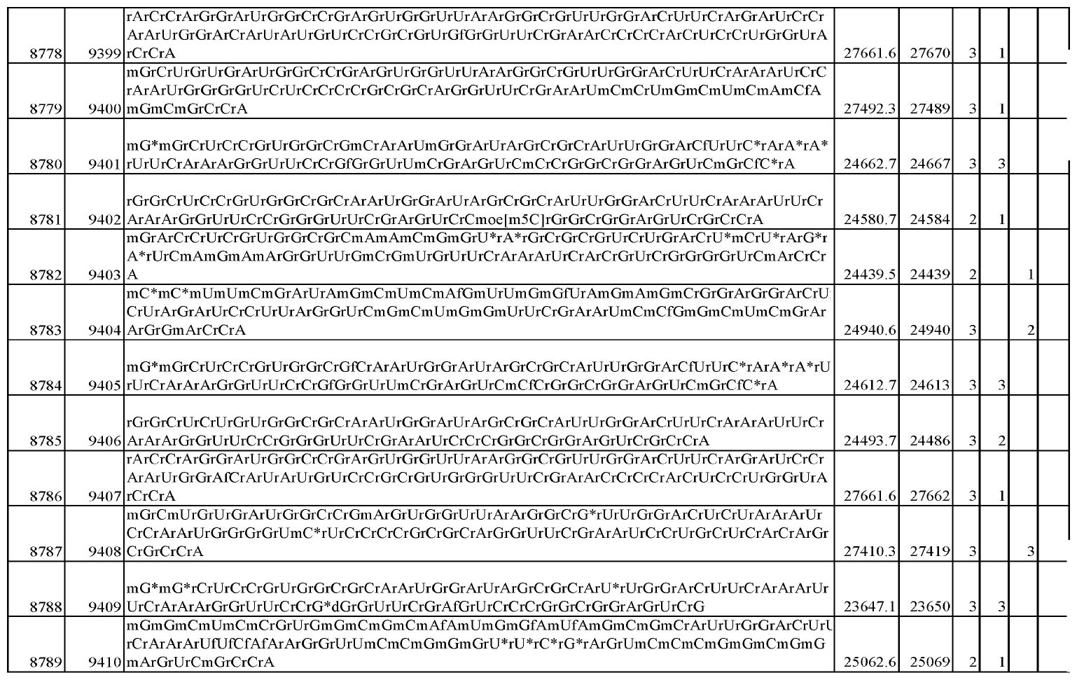

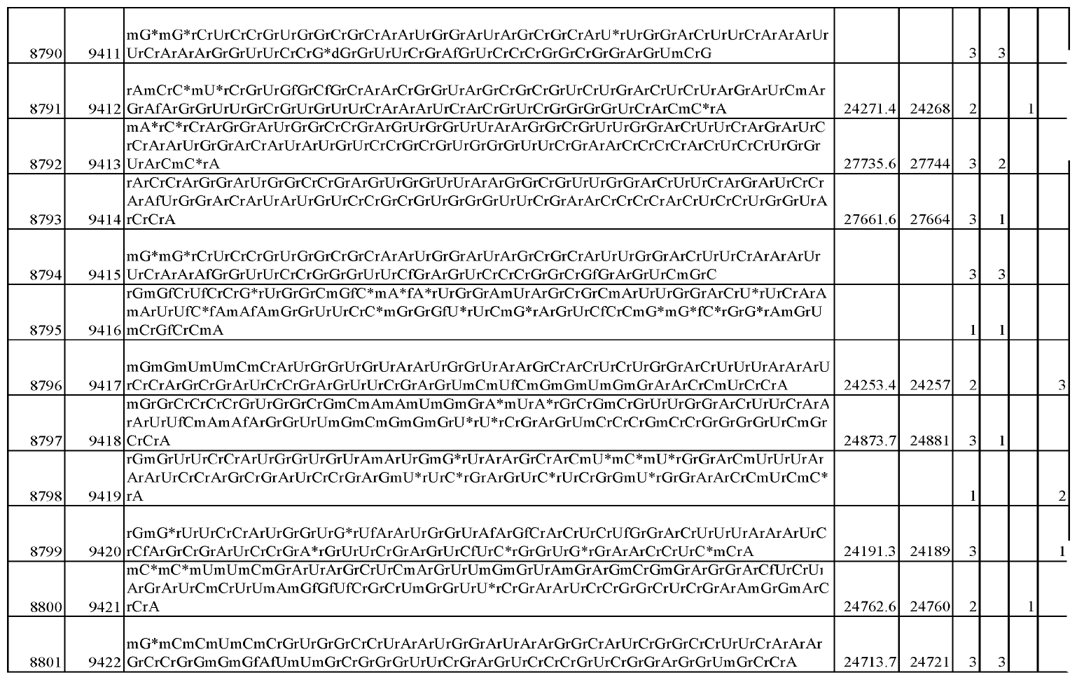

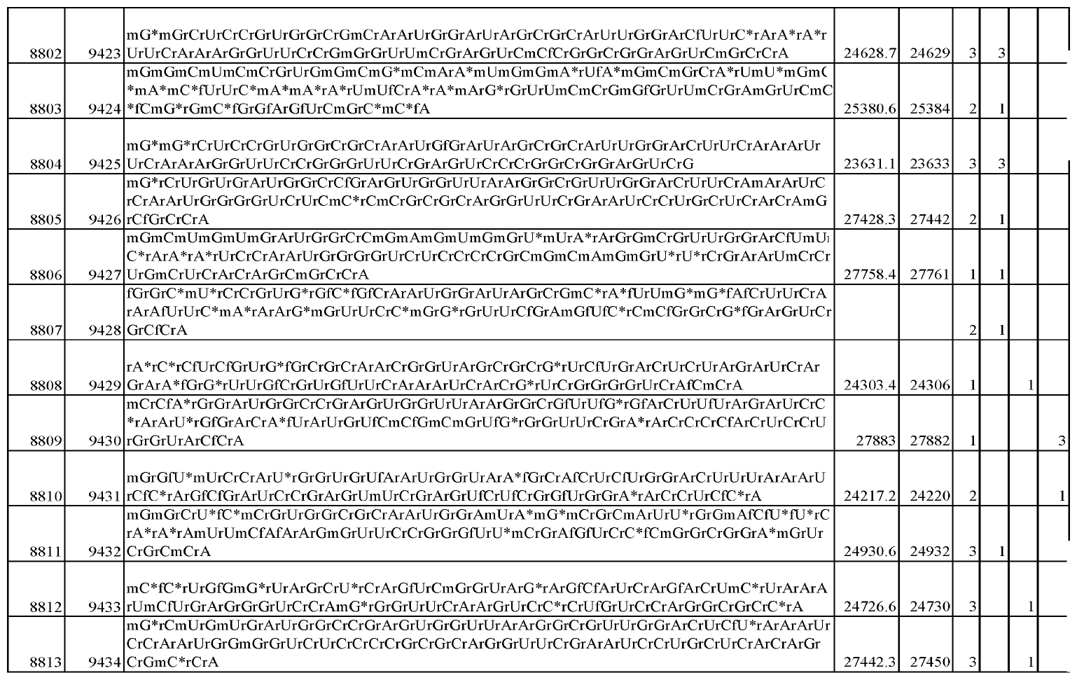

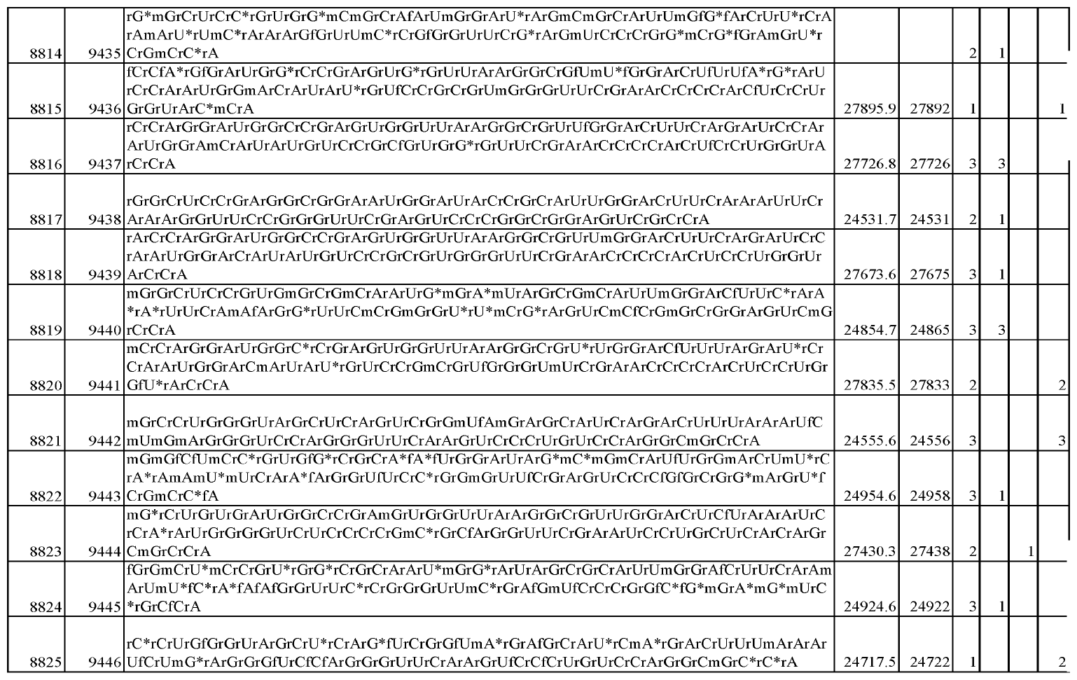

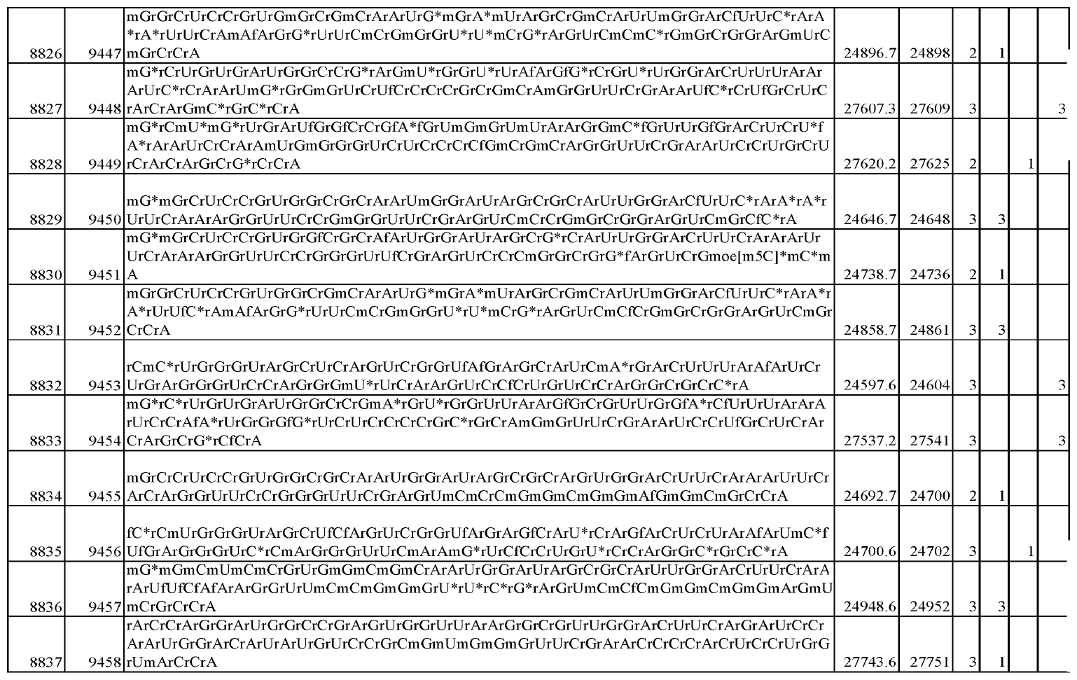

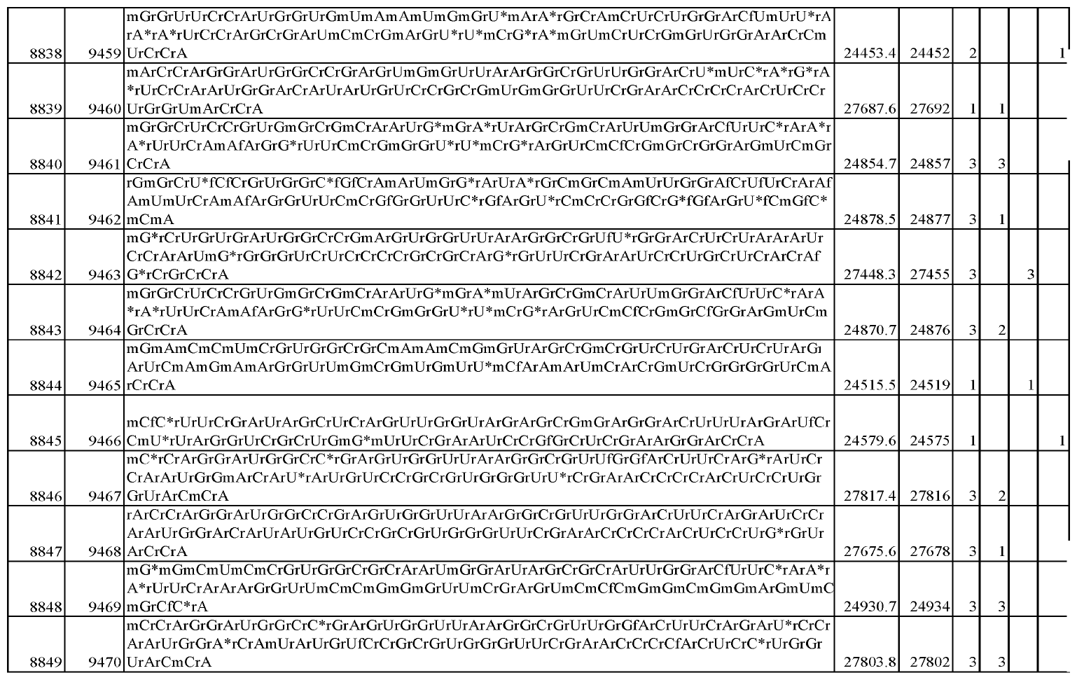

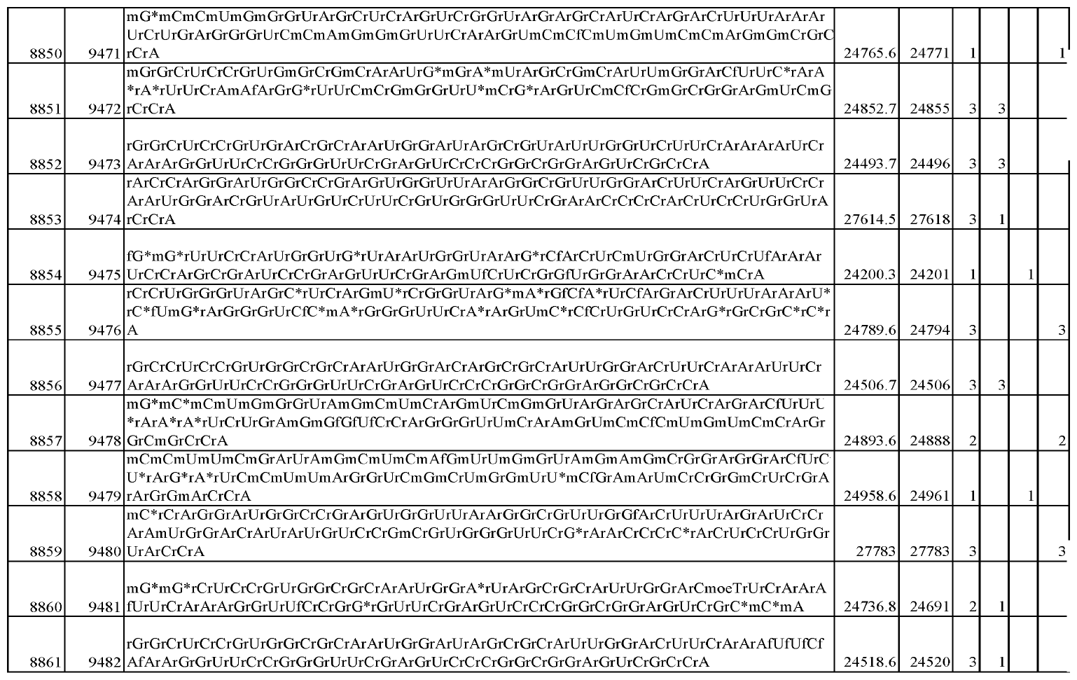

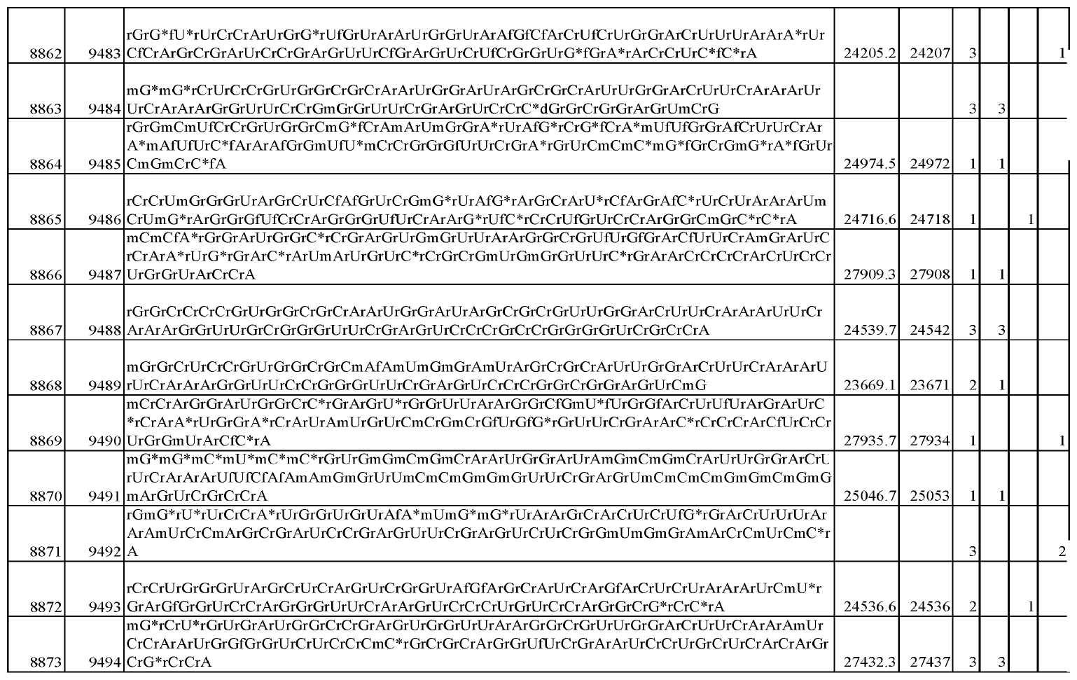

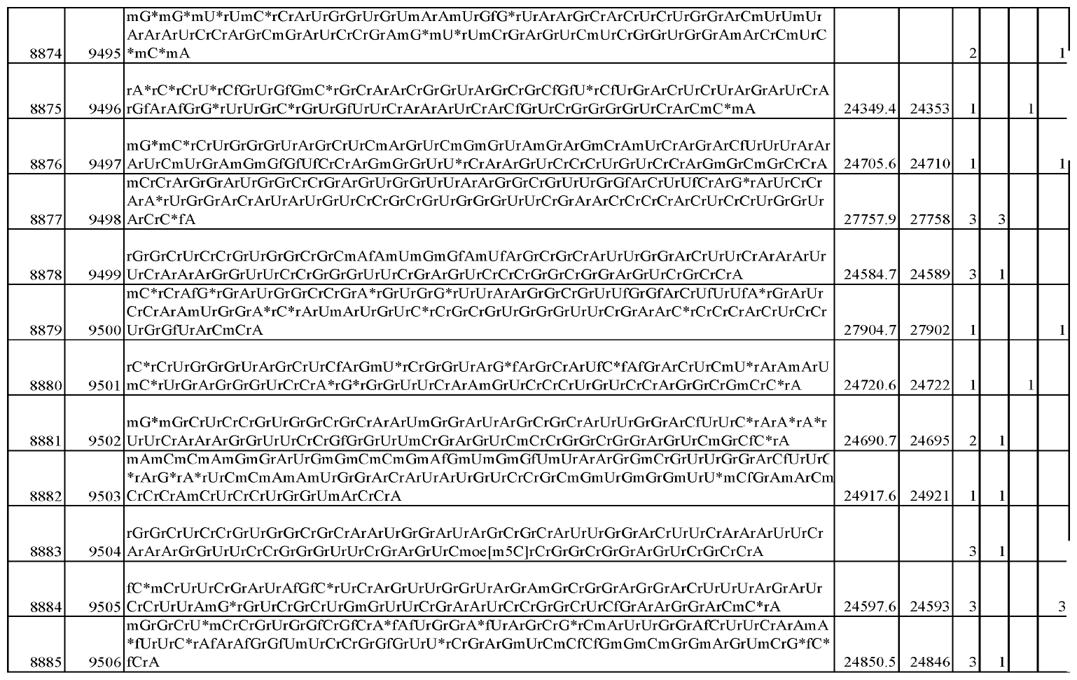

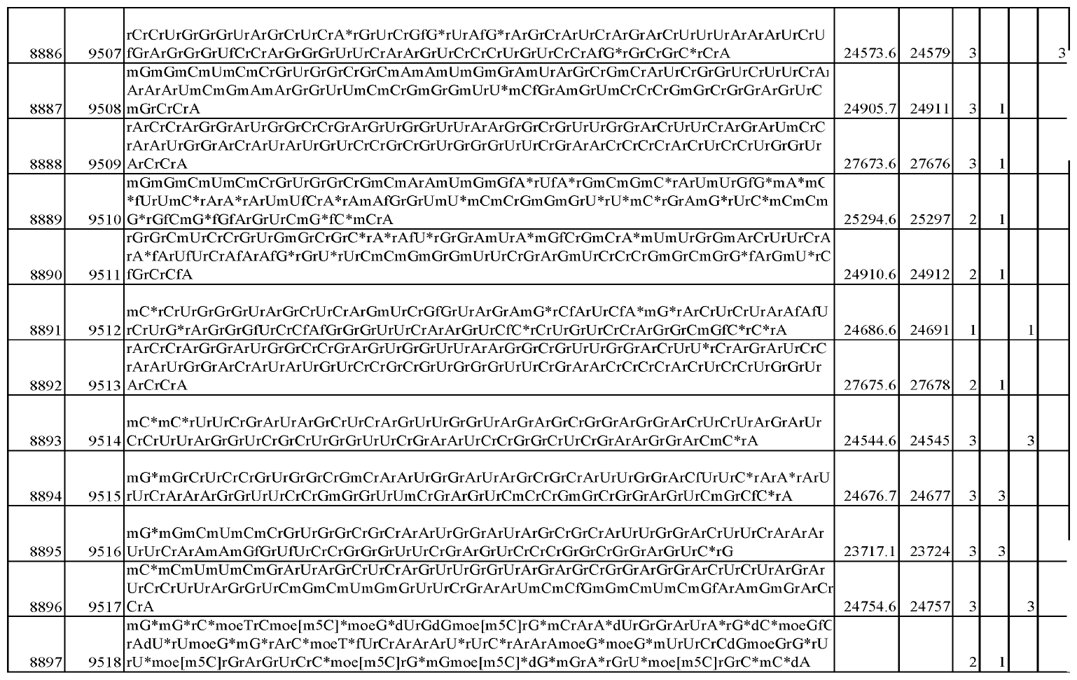

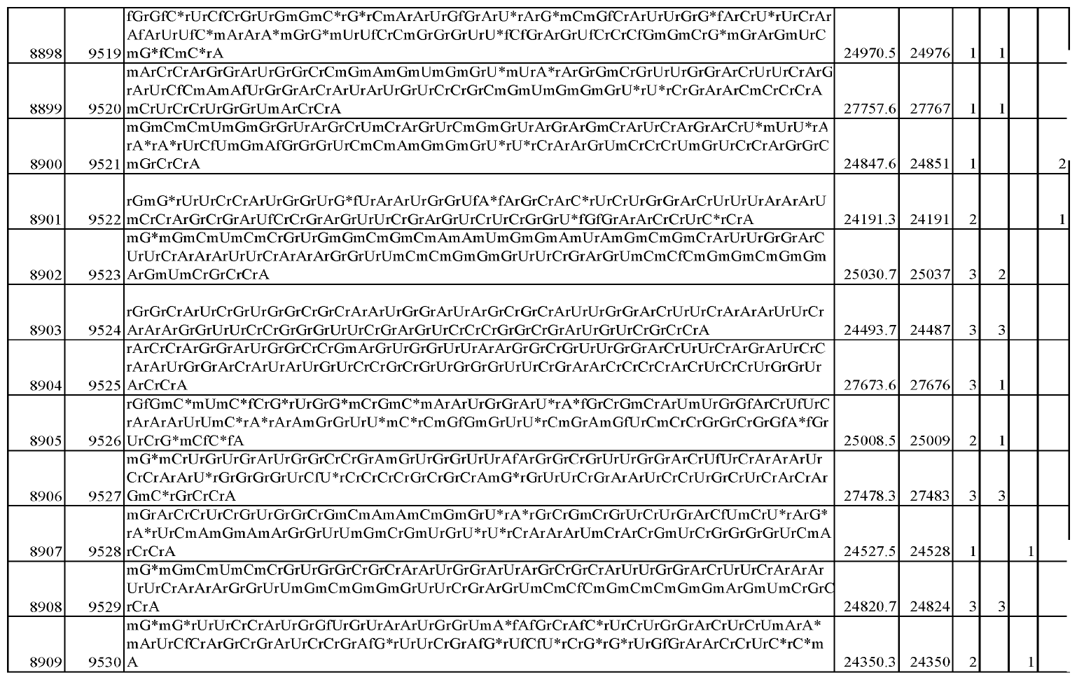

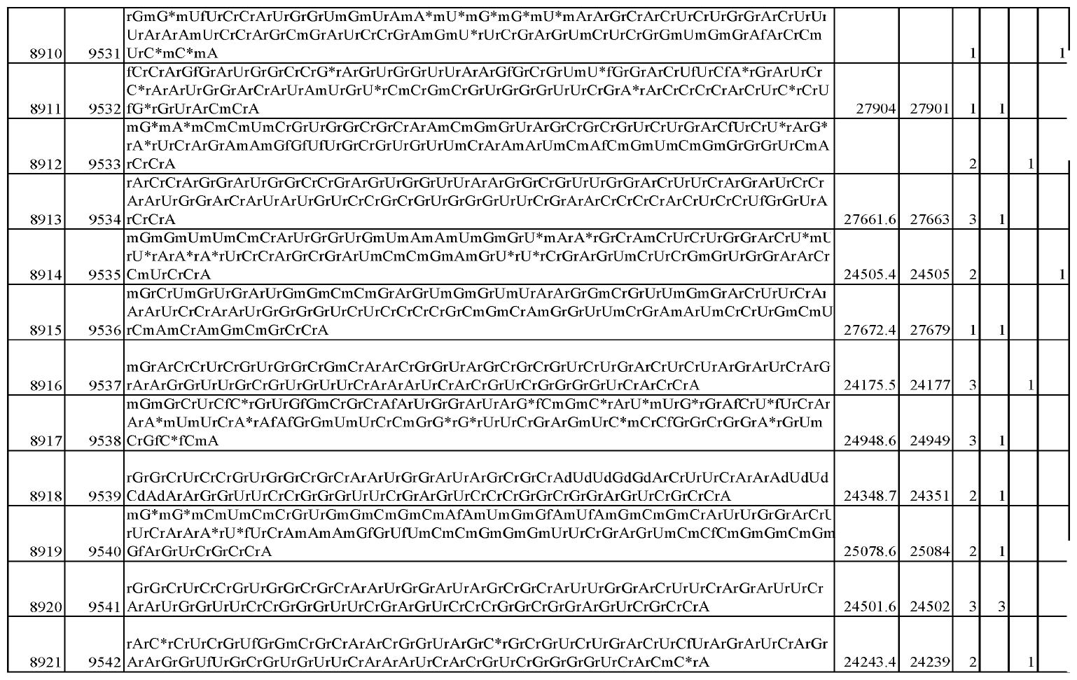

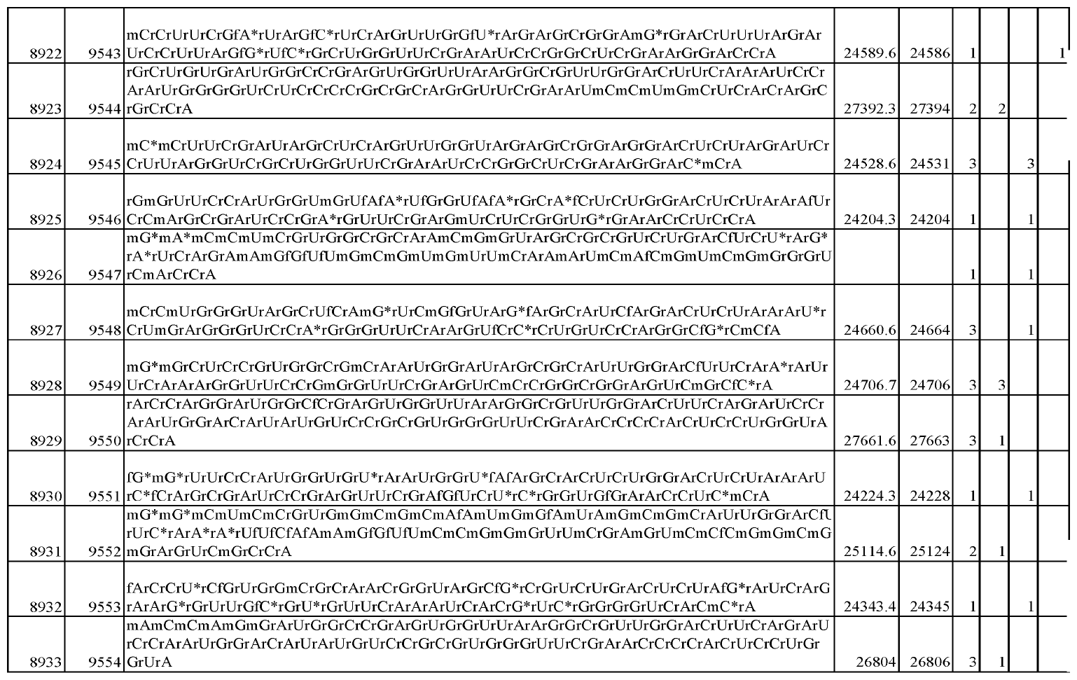

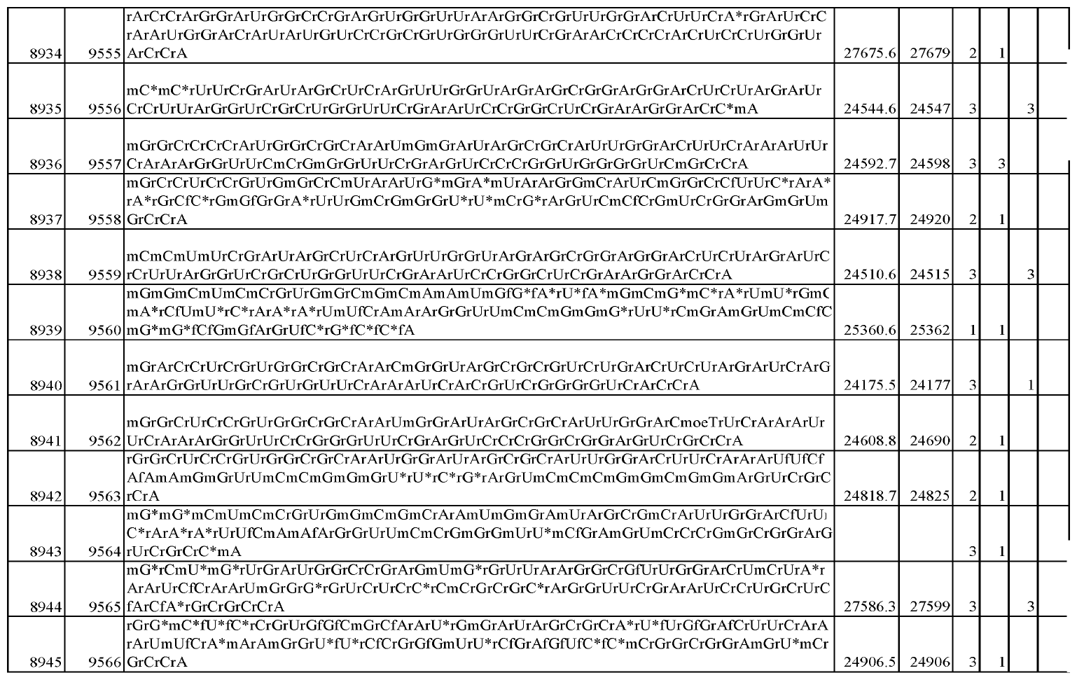

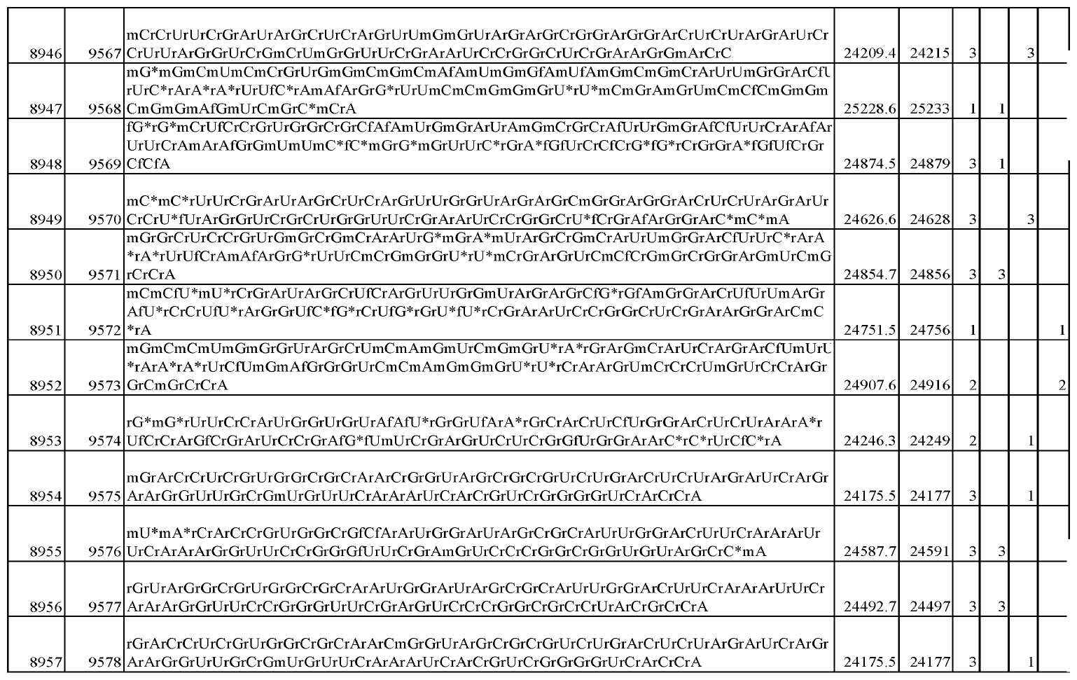

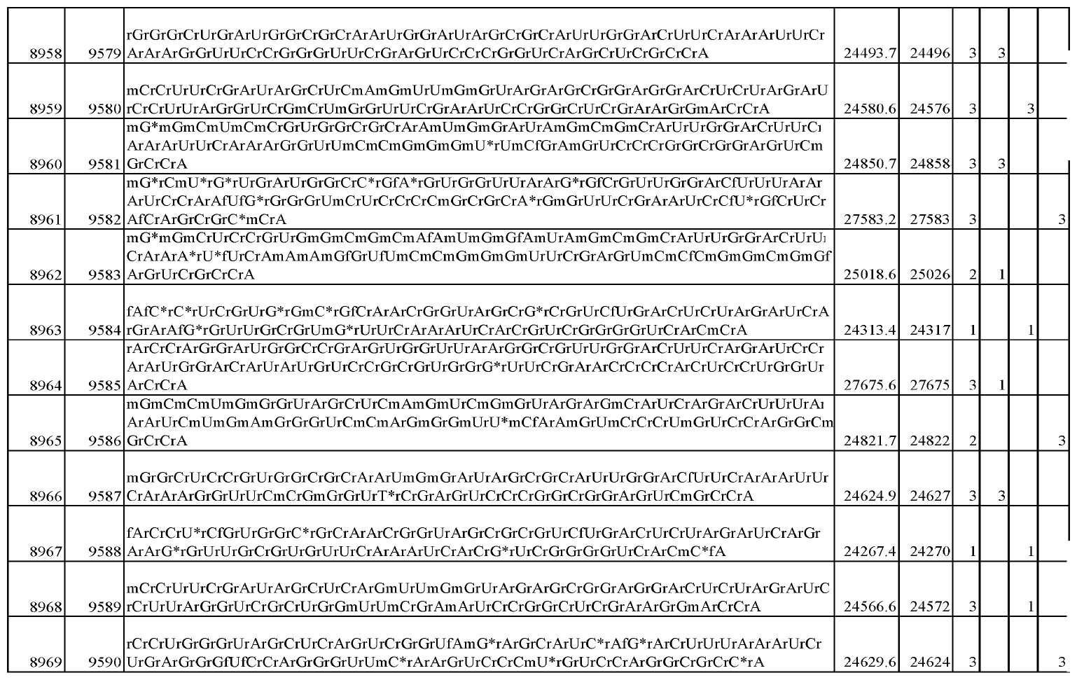

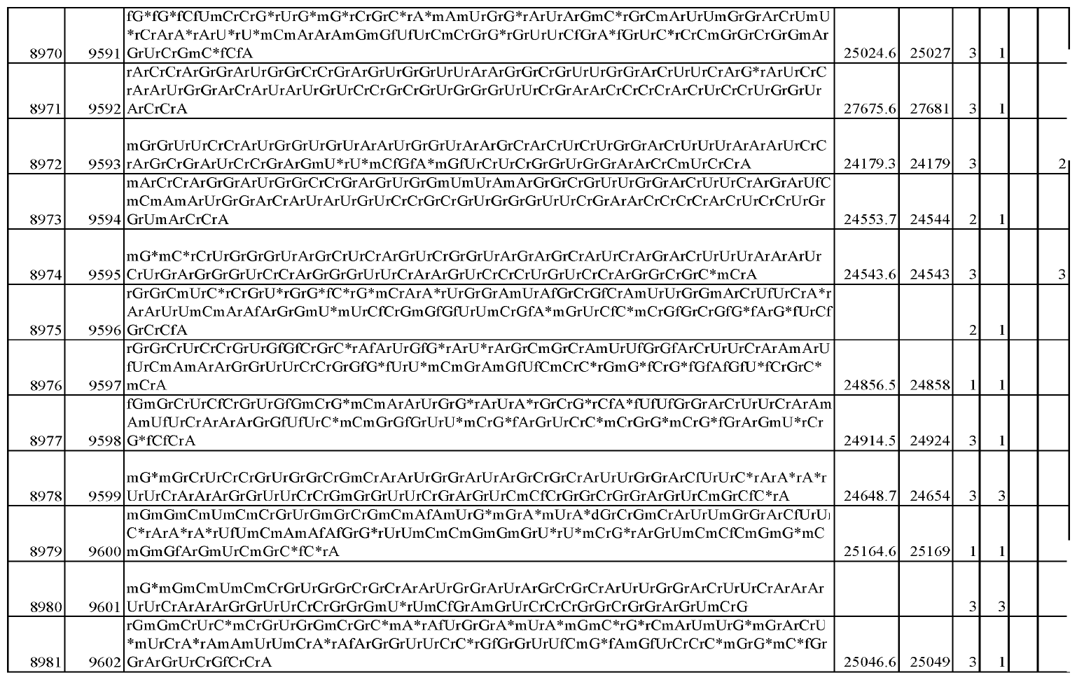

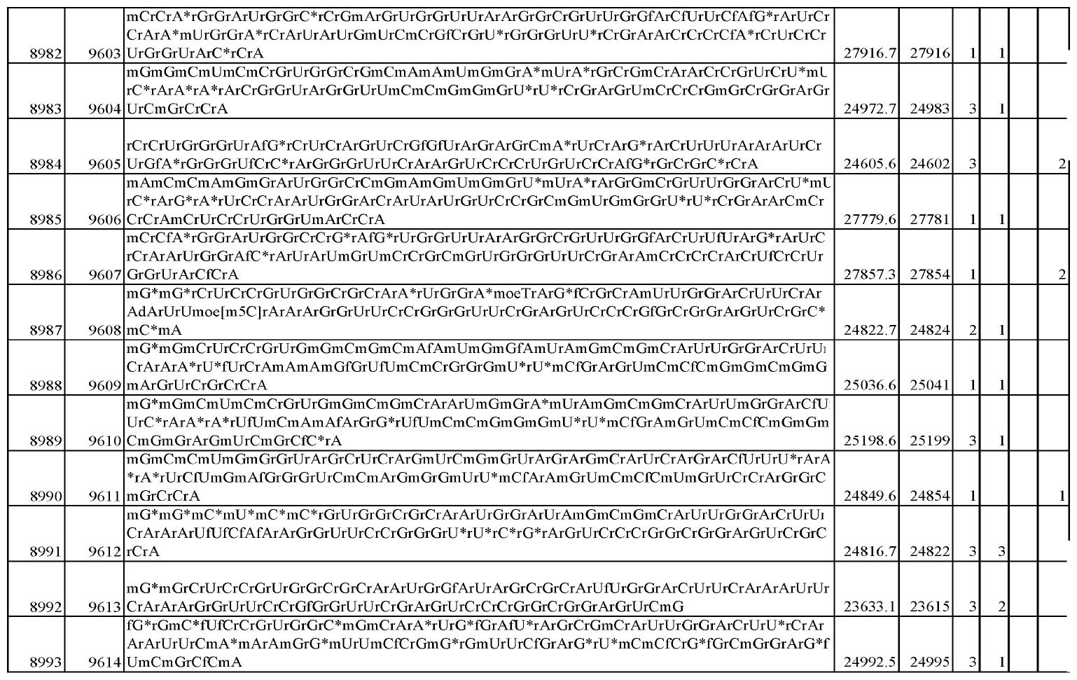

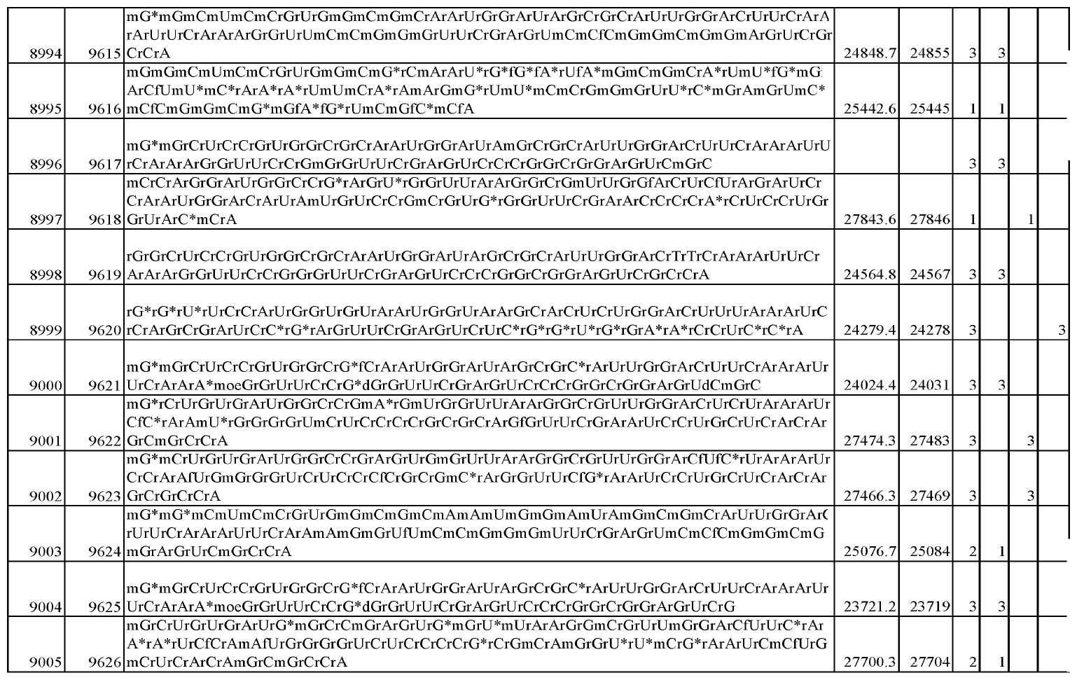

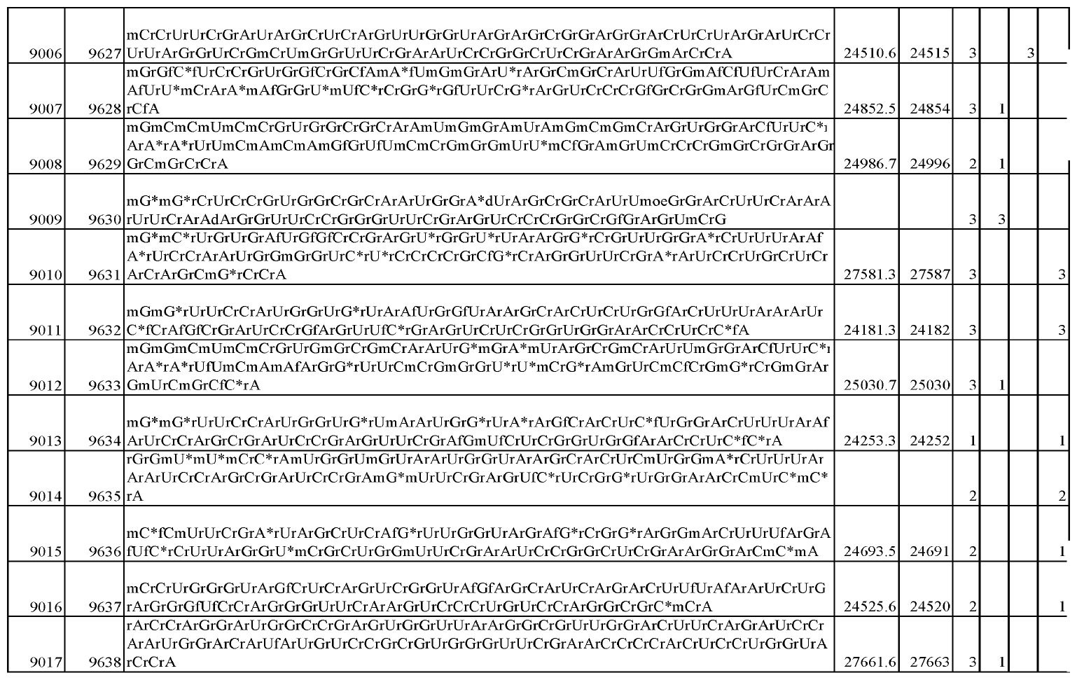

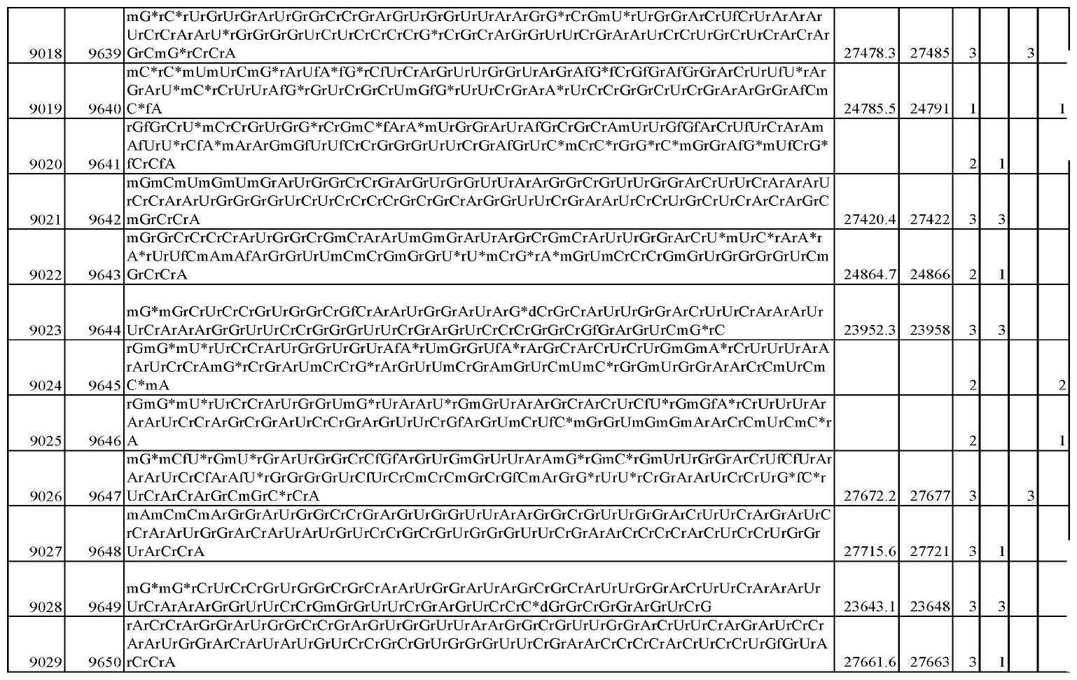

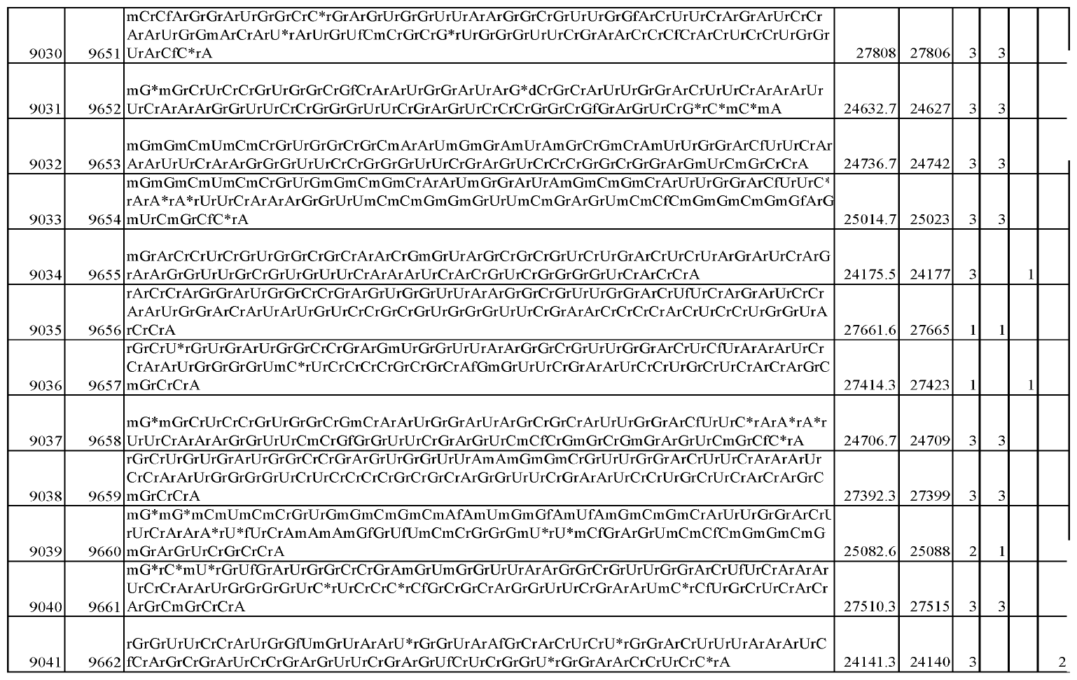

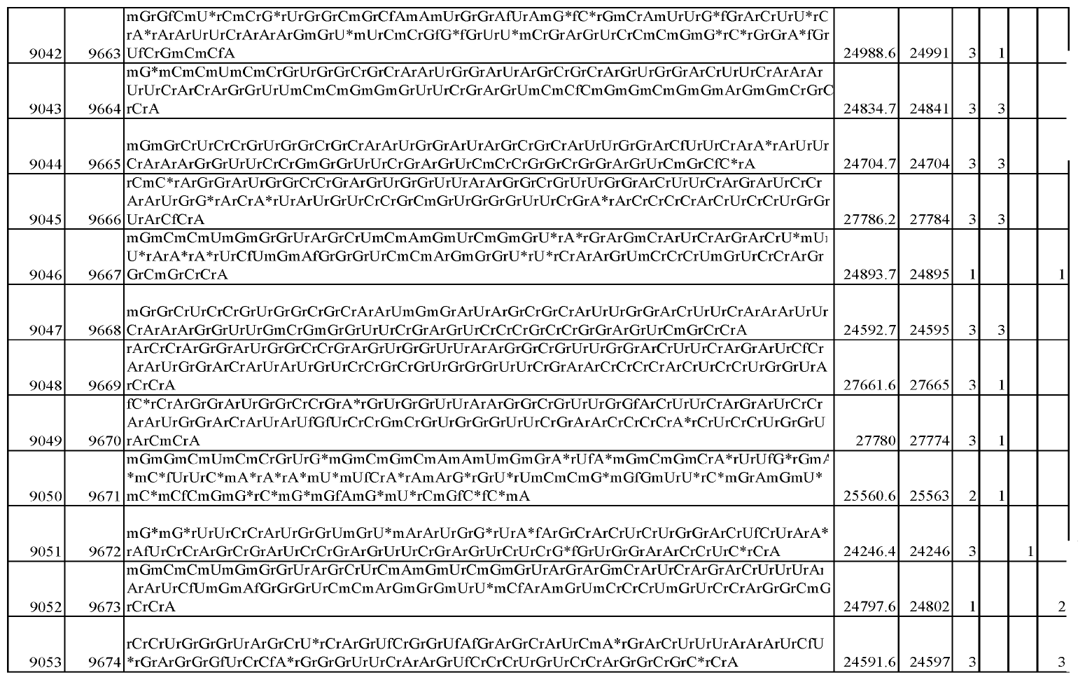

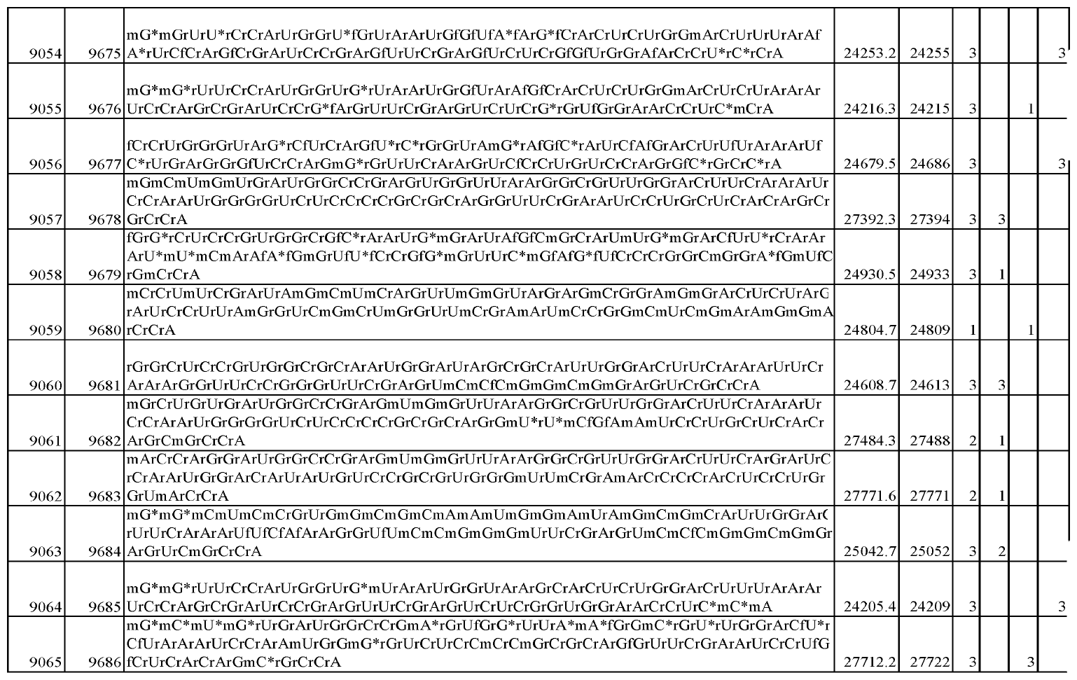

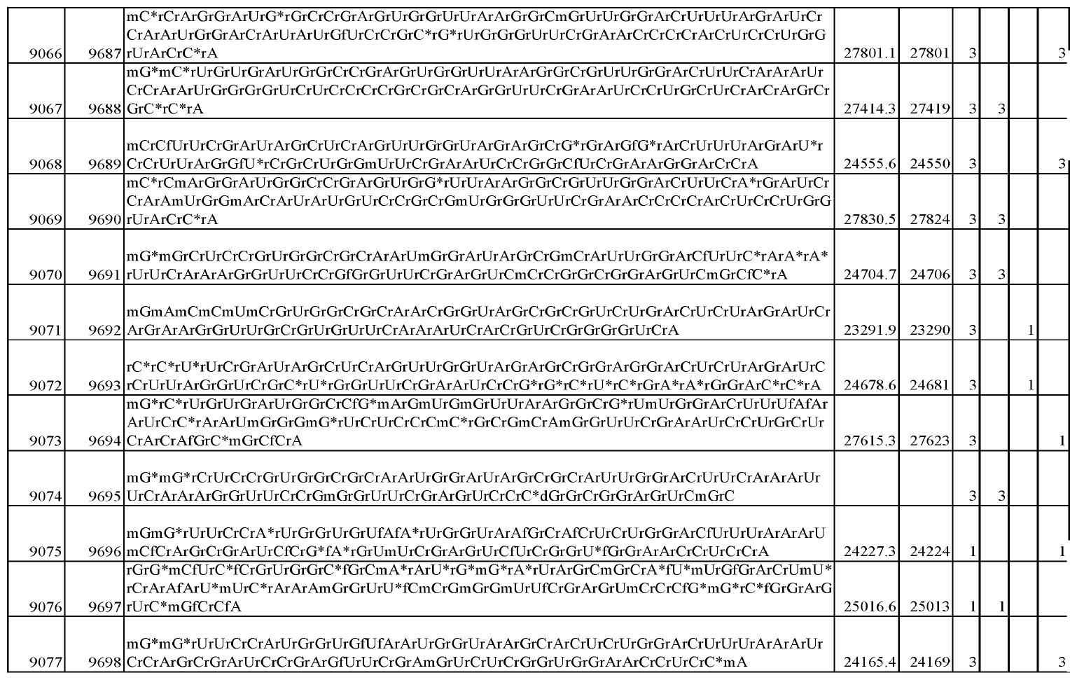

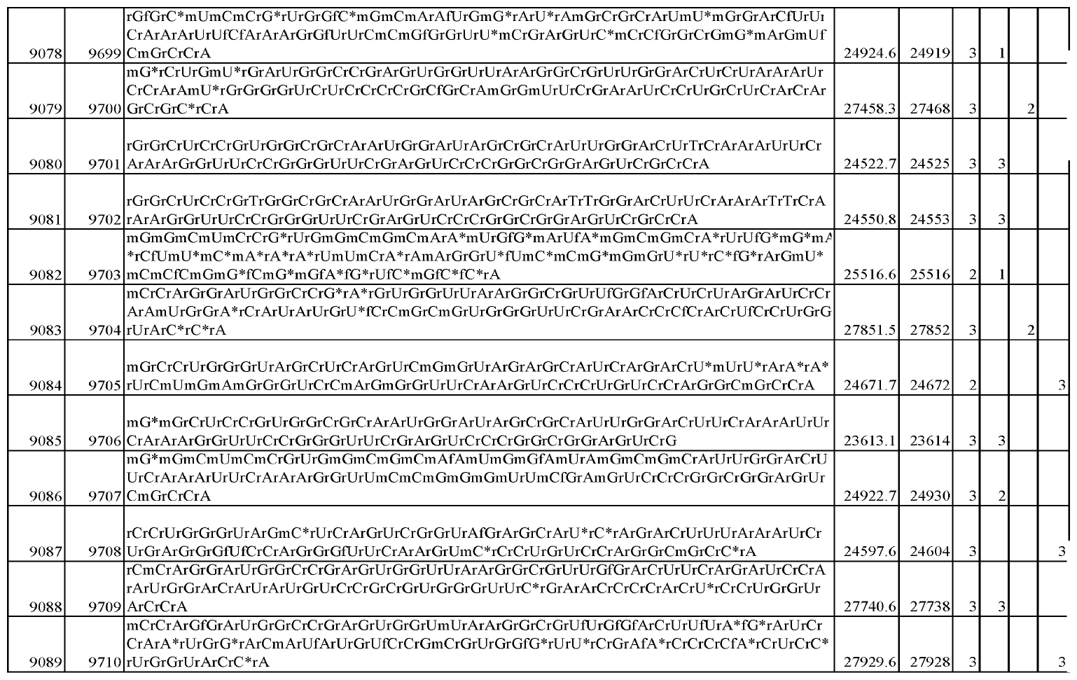

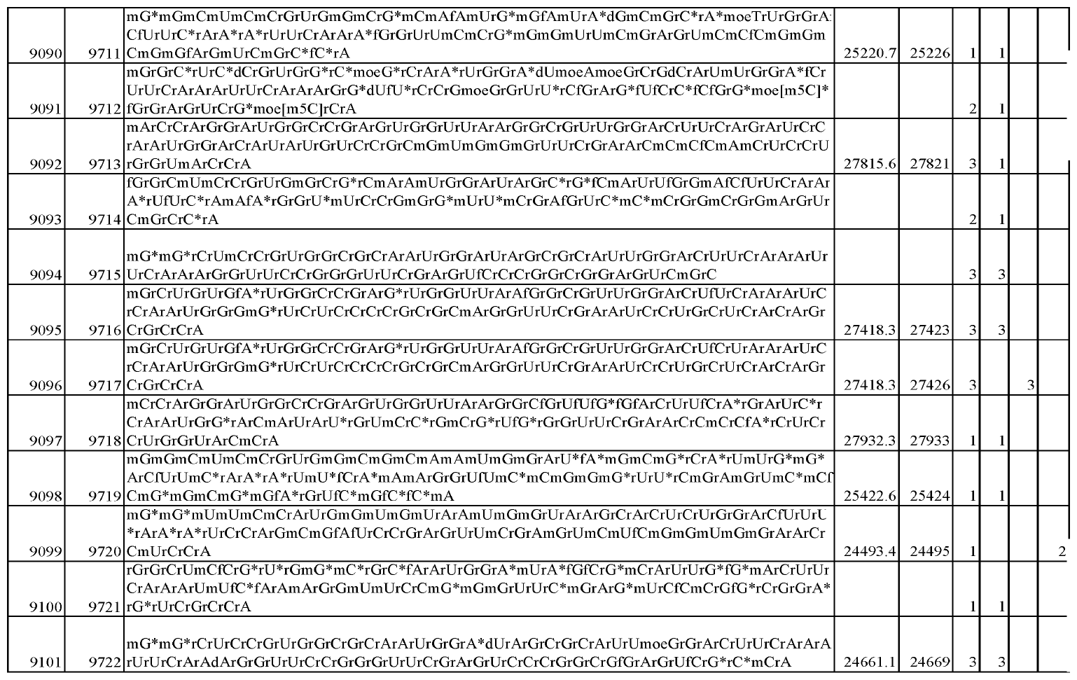

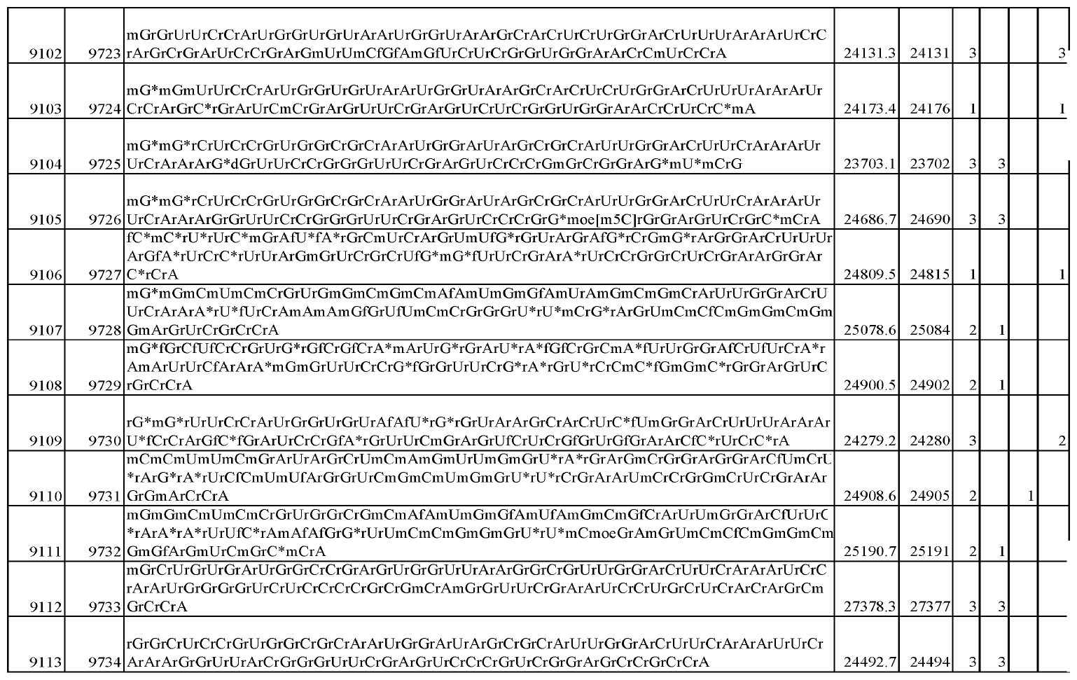

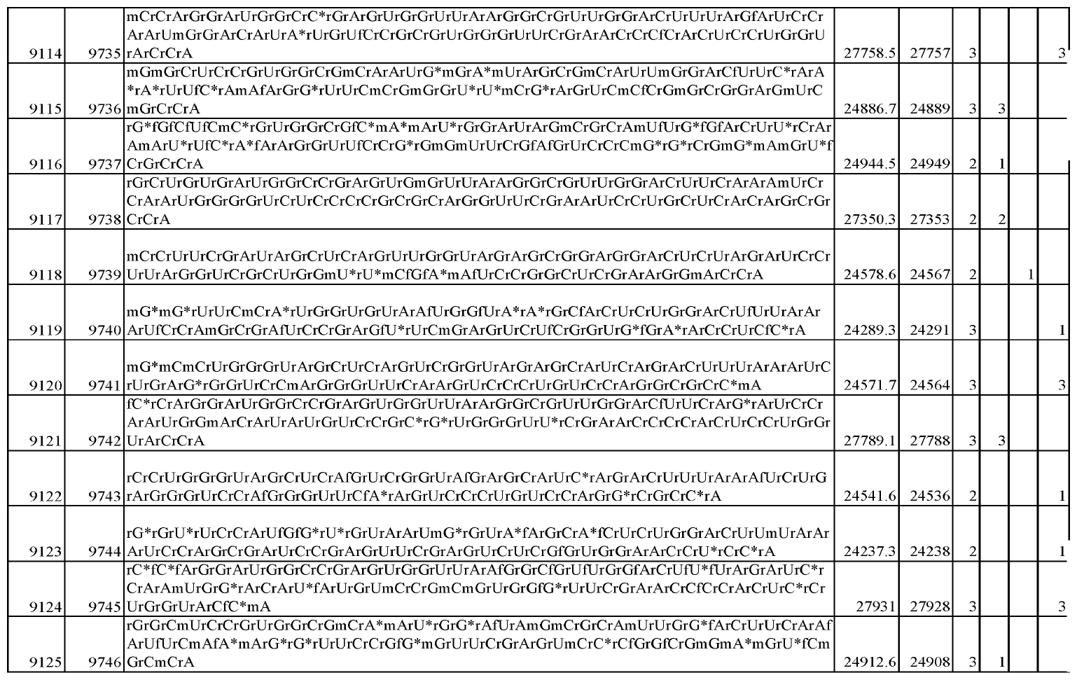

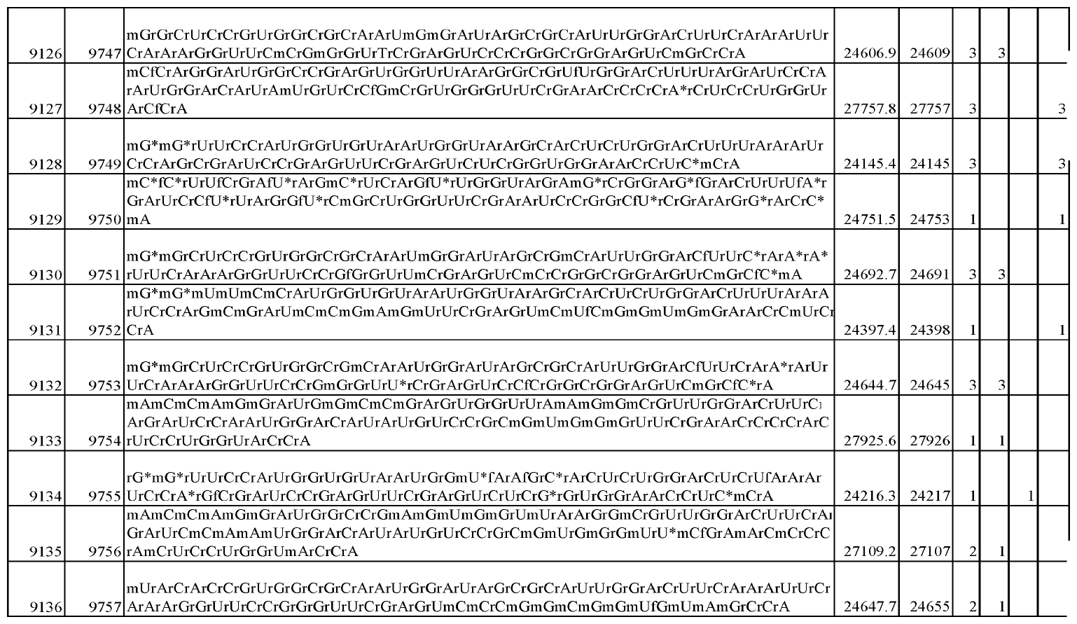

Hydrodynamic gene delivery (HGD) is a simple, fast, safe, and effective method for delivering transgenes in rodent models. A set of plasmids expressing both an cGFP-Luc-TGA reporter and and a TREM were designed. To evaluate tolerability and determine optimal plasmid concentration for maximum TREM delivery to the liver, the eGFP-WT Luc plasmid was administered to adult CD-1 mice via tail vein hydrodynamic injection at three doses: 10 ug, 30 ug, and 50 ug. As shown in FIG. 7A, plasmids in saline were successfully delivered to liver in a dose-dependent manner as shown by the luciferase readout signal. Next, 50 ug of DNA in saline (100 mg/kg) was administered to mice via tail vein hydrodynamic injuction to assess target engagement and PTC readthrough using either 1) eGFP-Nluc TGA reporter plasmid (PL-854), or 2) eGFP-Nluc WT reporter (PL1202), or 3) all-in-one plasmid eGFP-Nluc-TGA reporter with S-TAG (PL-1216), or 4) all-in-one plasmid eGFP-Nluc-TGA reporter with R-TGA (PL-1215). The Arg-TGA selectively rescued the TGA nonsense mutation in the reporter plasmid and showed a ˜1000-fold increase in luciferase signal compared to controls (FIG. 7B).

What is claimed is:

1. A tRNA effector molecule (TREM) comprising a sequence of Formula (I):
[L1]-[ASt Domain1]-[L2]-[DH Domain]-[L3]-[ACH Domain]-[VL Domain]-[TH Domain]-[L4]-[ASt Domain2] (I),
wherein:
independently, [L1] and [VL Domain], are optional; and
a nucleotide within [L1]-[ASt Domain1]-[L2] comprises a nucleotide having a non-naturally occurring modification.

2. The TREM of claim 1, wherein the non-naturally occurring modification is present on the 2′-position of a nucleotide sugar or within the internucleotide region (e.g., a backbone modification).

3. The TREM of any one of the preceding claims, wherein the non-naturally occurring modification is selected from a 2′-O-methyl (2-OMe), 2′-halo (e.g., 2′F or 2′Cl), 2′-O-methoxyethyl (2′MOE), or 2′deoxy modification.

4. The TREM of any one of the preceding claims, wherein the non-naturally occurring modification is a 2′OMe modification.

5. The TREM of any one of the preceding claims, wherein the non-naturally occurring modification is a 2′halo (e.g., 2′F or 2′Cl) modification.

6. The TREM of any one of the preceding claims, wherein the non-naturally occurring modification is a 2′MOE modification.

7. The TREM of any one of the preceding claims, wherein the non-naturally occurring modification is a 2′-deoxy modification.

8. The TREM of any one of the preceding claims, wherein the non-naturally occurring modification is present in the internucleotide region (e.g., a backbone modification).

9. The TREM of claim 8, wherein the non-naturally occurring modification is a phosphorothioate modification.

10. The TREM of any one of the preceding claims, wherein the TREM has a sequence selected from a sequence provided in FIG. 2.

11. The TREM of any one of the preceding claims, wherein the TREM is a TREM provided in FIG. 2.

12. The TREM of any one of the preceding claims, wherein the TREM comprises a TREM having at least 70%, 75%, 80%, 85%, 90%, 95%, or 99% sequence identity with a TREM provided in FIG. 2.

13. The TREM of any one of the preceding claims, wherein the TREM comprises a sequence that differs by no more than 1, 2, 3, 4, 5, 6, 7, 8, 9, or 10 nucleotides from a TREM provided in FIG. 2.

14. The TREM of any one of the preceding claims, wherein the TREM comprises 1, 2, 3, 4, 5, 6, 7, 8, 9, or 10 additional non-naturally occurring modifications compared with a TREM provided in FIG. 2 (e.g., 2′-ribose modifications or an internucleotide modification, e.g., 2′OMe, 2′-halo, 2′-MOE, 2′-deoxy, or phosphorothiorate modifications).

15. The TREM of any one of the preceding claims, wherein the TREM is selected from TREM NOs. 1-500, 501-1000, 1001-1500, 1501-2000, 2001-2500, 2501-3000, 3001-3500, 3501-4000, 4001-4500, 4501-5000, 5001-5500, 5501-6000, 6001-6500, 6501-7000, 7001-7500, 7501-8000, 8001-8500, 8501-9000, and 9001-9136 in FIG. 2.

16. The TREM of any one of the preceding claims, wherein the non-naturally occurring modification is present at a nucleotide position which corresponds to one or more of nucleotides 1-9 of SEQ ID NO: 622.

17. The TREM of any one of the preceding claims, wherein:
(i) the non-naturally occurring modification is present at a nucleotide position which corresponds to one or more of nucleotides 1-9 of SEQ ID NO: 622; and/or
(ii) the TREM differs from the nucleotide sequence of SEQ ID NO: 622 by no more than 1, 2, 3, 4, 5, 6, 7, 8, 9, or 10 nucleotides nucleotides.

18. The TREM of any one of the preceding claims, wherein:
(i) the non-naturally occurring modification is present at a nucleotide position which corresponds to one or more of nucleotides 1-9 of SEQ ID NO: 622; and/or
(ii) the TREM comprises the nucleotide sequence of SEQ ID NO: 622.

19. The TREM of any one of claims 1-17, wherein the TREM comprises the nucleotide sequence of SEQ ID NO: 623.

20. The TREM of any one of claims 1-17, wherein the TREM comprises the nucleotide sequence of SEQ ID NO: 624.

21. The TREM of any one of the preceding claims, wherein the non-naturally occurring modification is present at a nucleotide position which corresponds to one or more of nucleotides 1-9, according to the CtNS.

22. A tRNA effector molecule (TREM) comprising a sequence of Formula (I):
[L1]-[ASt Domain1]-[L2]-[DH Domain]-[L3]-[ACH Domain]-[VL Domain]-[TH Domain]-[L4]-[ASt Domain2] (I),
wherein:
independently, [L1] and [VL Domain], are optional; and
a nucleotide within [DH Domain1], or [L3] comprises a nucleotide having a non-naturally occurring modification.

23. The TREM of claim 22, wherein the non-naturally occurring modification is present on the 2′-position of a nucleotide sugar or within the internucleotide region (e.g., a backbone modification).

24. The TREM of any one of claims 22-23, wherein the non-naturally occurring modification is selected from a 2′-O-methyl (2-OMe), 2′-halo (e.g., 2′F or 2′Cl), 2′-O-methoxyethyl (2′MOE), or 2′deoxy modification.

25. The TREM of any one of claims 22-24, wherein the non-naturally occurring modification is a 2′OMe modification.

26. The TREM of any one of claims 22-25, wherein the non-naturally occurring modification is a 2′halo (e.g., 2′F or 2′Cl) modification.

27. The TREM of any one of claims 22-26, wherein the non-naturally occurring modification is a 2′MOE modification.

28. The TREM of any one of claims 22-27, wherein the non-naturally occurring modification is a 2′-deoxy modification.

29. The TREM of any one of claims 22-28, wherein the non-naturally occurring modification is present in the internucleotide region (e.g., a backbone modification).

30. The TREM of claim 29, wherein the non-naturally occurring modification is a phosphorothioate modification.

31. The TREM of any one of claims 22-30, wherein the TREM has a sequence selected from a sequence provided in FIG. 2.

32. The TREM of any one of claims 22-31, wherein the TREM is a TREM provided in FIG. 2.

33. The TREM of any one of claims 22-32, wherein the TREM comprises a TREM having at least 70%, 75%, 80%, 85%, 90%, 95%, or 99% sequence identity with a TREM provided in FIG. 2.

34. The TREM of any one of claims 22-33, wherein the TREM comprises a sequence that differs by no more than 1, 2, 3, 4, 5, 6, 7, 8, 9, or 10 nucleotides from a TREM provided in FIG. 2.

35. The TREM of any one of claims 22-34, wherein the TREM comprises 1, 2, 3, 4, 5, 6, 7, 8, 9, or 10 additional non-naturally occurring modifications compared with a TREM provided in FIG. 2 (e.g., 2′-ribose modifications or an internucleotide modification, e.g., 2′OMe, 2′-halo, 2′-MOE, 2′-deoxy, or phosphorothiorate modifications).

36. The TREM of any one of claims 22-35, wherein the TREM is selected from TREM NO. 1-500, 501-1000, 1001-1500, 1501-2000, 2001-2500, 2501-3000, 3001-3500, 3501-4000, 4001-4500, 4501-5000, 5001-5500, 5501-6000, 6001-6500, 6501-7000, 7001-7500, 7501-8000, 8001-8500, 8501-9000, and 9001-9136 in FIG. 2in FIG. 2.

37. The TREM of any one of claims 22-3623-37, wherein the non-naturally occurring modification is present at a nucleotide position which corresponds to one or more of nucleotides 10-26 of SEQ ID NO: 622.

38. The TREM of any one of claims 22-37, wherein:
(i) the non-naturally occurring modification is present at a nucleotide position which corresponds to one or more of nucleotides 10-26 of SEQ ID NO: 622; and/or
(ii) the TREM differs from the nucleotide sequence of SEQ ID NO: 622 by no more than 1, 2, 3, 4, 5, 6, 7, 8, 9, or 10 nucleotides nucleotides.

39. The TREM of any one of claims 22-38, wherein:
(i) the non-naturally occurring modification is present at a nucleotide position which corresponds to one or more of nucleotides 10-26 of SEQ ID NO: 622; and/or
(ii) the TREM comprises the nucleotide sequence of SEQ ID NO: 622.

40. The TREM of any one of claims 22-38, wherein the TREM comprises the nucleotide sequence of SEQ ID NO: 623.

41. The TREM of any one of claims 22-38, wherein the TREM comprises the nucleotide sequence of SEQ ID NO: 624.

42. The TREM of any one of claims 22-41, wherein the non-naturally occurring modification is present at a nucleotide position which corresponds to one or more of nucleotides 10-26, according to the CtNS.

43. A tRNA effector molecule (TREM) comprising a sequence of Formula (I):
[L1]-[ASt Domain1]-[L2]-[DH Domain]-[L3]-[ACH Domain]-[VL Domain]-[TH Domain]-[L4]-[ASt Domain2] (I),
wherein:
independently, [L1] and [VL Domain], are optional; and
a nucleotide within [ACH Domain] comprises a nucleotide having a non-naturally occurring modification.

44. The TREM of claim 43, wherein the non-naturally occurring modification is present on the 2′-position of a nucleotide sugar or within the internucleotide region (e.g., a backbone modification).

45. The TREM of any one of claims 43-44, wherein the non-naturally occurring modification is selected from a 2′-O-methyl (2-OMe), 2′-halo (e.g., 2′F or 2′Cl), 2′-O-methoxyethyl (2′MOE), or 2′deoxy modification.

46. The TREM of any one of claims 43-45, wherein the non-naturally occurring modification is a 2′OMe modification.

47. The TREM of any one of claims 43-46, wherein the non-naturally occurring modification is a 2′halo (e.g., 2′F or 2′Cl) modification.

48. The TREM of any one of claims 43-47, wherein the non-naturally occurring modification is a 2′MOE modification.

49. The TREM of any one of claims 43-48, wherein the non-naturally occurring modification is a 2′-deoxy modification.

50. The TREM of any one of claims 43-49, wherein the non-naturally occurring modification is present in the internucleotide region (e.g., a backbone modification).

51. The TREM of claim 50, wherein the non-naturally occurring modification is a phosphorothioate modification.

52. The TREM of any one of claims 43-51, wherein the TREM has a sequence selected from a sequence provided in FIG. 2.

53. The TREM of any one of claims 43-52, wherein the TREM is a TREM provided in FIG. 2.

54. The TREM of any one of claims 43-53, wherein the TREM comprises a TREM having at least 70%, 75%, 80%, 85%, 90%, 95%, or 99% sequence identity with a TREM provided in FIG. 2.

55. The TREM of any one of claims 43-54, wherein the TREM comprises a sequence that differs by no more than 1, 2, 3, 4, 5, 6, 7, 8, 9, or 10 nucleotides from a TREM provided in FIG. 2.

56. The TREM of any one of claims 43-55, wherein the TREM comprises 1, 2, 3, 4, 5, 6, 7, 8, 9, or 10 additional non-naturally occurring modifications compared with a TREM provided in FIG. 2 (e.g., 2′-ribose modifications or an internucleotide modification, e.g., 2′OMe, 2′-halo, 2′-MOE, 2′-deoxy, or phosphorothiorate modifications).

57. The TREM of any one of claims 43-56, wherein the TREM is selected from TREM NO. 1-500, 501-1000, 1001-1500, 1501-2000, 2001-2500, 2501-3000, 3001-3500, 3501-4000, 4001-4500, 4501-5000, 5001-5500, 5501-6000, 6001-6500, 6501-7000, 7001-7500, 7501-8000, 8001-8500, 8501-9000, and 9001-9136 in FIG. 2.

58. The TREM of any one of claims 43-57, wherein the non-naturally occurring modification is present at a nucleotide position which corresponds to one or more of nucleotides 27-43 of SEQ ID NO: 622.

59. The TREM of any one of claims 43-58, wherein:
(i) the non-naturally occurring modification is present at a nucleotide position which corresponds to one or more of nucleotides 27-43 of SEQ ID NO: 622; and/or
(ii) the TREM differs from the nucleotide sequence of SEQ ID NO: 622 by no more than 1, 2, 3, 4, 5, 6, 7, 8, 9, or 10 nucleotides nucleotides.

60. The TREM of any one of claims 43-59, wherein:
(i) the non-naturally occurring modification is present at a nucleotide position which corresponds to one or more of nucleotides 27-43 of SEQ ID NO: 622; and/or
(ii) the TREM comprises the nucleotide sequence of SEQ ID NO: 622.

61. The TREM of any one of claims 43-59, wherein the TREM comprises the nucleotide sequence of SEQ ID NO: 623.

62. The TREM of any one of claims 43-59, wherein the TREM comprises the nucleotide sequence of SEQ ID NO: 624.

63. The TREM of any one of claims 43-62, wherein the non-naturally occurring modification is present at a nucleotide position which corresponds to one or more of nucleotides 27-43, according to the CtNS.

64. A tRNA effector molecule (TREM) comprising a sequence of Formula (I):
[L1]-[ASt Domain1]-[L2]-[DH Domain]-[L3]-[ACH Domain]-[VL Domain]-[TH Domain]-[L4]-[ASt Domain2] (I),
wherein:
independently, [L1] and [VL Domain], are optional; and
a nucleotide within [VL Domain] comprises a nucleotide having a non-naturally occurring modification.

65. The TREM of claim 64, wherein the non-naturally occurring modification is present on the 2′-position of a nucleotide sugar or within the internucleotide region (e.g., a backbone modification).

66. The TREM of any one of claims 64-65, wherein the non-naturally occurring modification is selected from a 2′-O-methyl (2-OMe), 2′-halo (e.g., 2′F or 2′Cl), 2′-O-methoxyethyl (2′MOE), or 2′deoxy modification.

67. The TREM of any one of claims 64-66, wherein the non-naturally occurring modification is a 2′OMe modification.

68. The TREM of any one of claims 64-67, wherein the non-naturally occurring modification is a 2′halo (e.g., 2′F or 2′Cl) modification.

69. The TREM of any one of claims 64-68, wherein the non-naturally occurring modification is a 2′MOE modification.

70. The TREM of any one of claims 64-69, wherein the non-naturally occurring modification is a 2′-deoxy modification.

71. The TREM of any one of claims 64-70, wherein the non-naturally occurring modification is present in the internucleotide region (e.g., a backbone modification).

72. The TREM of claim 71, wherein the non-naturally occurring modification is a phosphorothioate modification.

73. The TREM of any one of claims 64-72, wherein the TREM has a sequence selected from a sequence provided in FIG. 2.

74. The TREM of any one of claims 64-73, wherein the TREM is a TREM provided in FIG. 2.

75. The TREM of any one of claims 64-74, wherein the TREM comprises a TREM having at least 70%, 75%, 80%, 85%, 90%, 95%, or 99% sequence identity with a TREM provided in FIG. 2.

76. The TREM of any one of claims 64-75, wherein the TREM comprises a sequence that differs by no more than 1, 2, 3, 4, 5, 6, 7, 8, 9, or 10 nucleotides from a TREM provided in FIG. 2.

77. The TREM of any one of claims 64-76, wherein the TREM comprises 1, 2, 3, 4, 5, 6, 7, 8, 9, or 10 additional non-naturally occurring modifications compared with a TREM provided in FIG. 2 (e.g., 2′-ribose modifications or an internucleotide modification, e.g., 2′OMe, 2′-halo, 2′-MOE, 2′-deoxy, or phosphorothiorate modifications).

78. The TREM of any one of claims 64-77, wherein the TREM is selected from TREM NO. 1-500, 501-1000, 1001-1500, 1501-2000, 2001-2500, 2501-3000, 3001-3500, 3501-4000, 4001-4500, 4501-5000, 5001-5500, 5501-6000, 6001-6500, 6501-7000, 7001-7500, 7501-8000, 8001-8500, 8501-9000, and 9001-9136 in FIG. 2.

79. The TREM of any one of claims 64-78, wherein the non-naturally occurring modification is present at a nucleotide position which corresponds to one or more of nucleotides 44-48 of SEQ ID NO: 622.

80. The TREM of any one of claims 64-79, wherein:
(i) the non-naturally occurring modification is present at a nucleotide position which corresponds to one or more of nucleotides 44-48 of SEQ ID NO: 622; and/or
(ii) the TREM differs from the nucleotide sequence of SEQ ID NO: 622 by no more than 1, 2, 3, 4, 5, 6, 7, 8, 9, or 10 nucleotides nucleotides.

81. The TREM of any one of claims 64-80, wherein:
(i) the non-naturally occurring modification is present at a nucleotide position which corresponds to one or more of nucleotides 44-48 of SEQ ID NO: 622; and/or
(ii) the TREM comprises the nucleotide sequence of SEQ ID NO: 622.

82. The TREM of any one of claims 64-80, wherein the TREM comprises the nucleotide sequence of SEQ ID NO: 623.

83. The TREM of any one of claims 64-80, wherein the TREM comprises the nucleotide sequence of SEQ ID NO: 624.

84. The TREM of any one of claims 64-83, wherein the non-naturally occurring modification is present at a nucleotide position which corresponds to one or more of nucleotides 44-48, according to the CtNS.

85. A tRNA effector molecule (TREM) comprising a sequence of Formula (I):
[L1]-[ASt Domain1]-[L2]-[DH Domain]-[L3]-[ACH Domain]-[VL Domain]-[TH Domain]-[L4]-[ASt Domain2] (I),
wherein:
independently, [L1] and [VL Domain], are optional; and
a nucleotide within [TH Domain] comprises a nucleotide having a non-naturally occurring modification.

86. The TREM of claim 85, wherein the non-naturally occurring modification is present on the 2′-position of a nucleotide sugar or within the internucleotide region (e.g., a backbone modification).

87. The TREM of any one of claims 85-86, wherein the non-naturally occurring modification is selected from a 2′-O-methyl (2-OMe), 2′-halo (e.g., 2′F or 2′Cl), 2′-O-methoxyethyl (2′MOE), or 2′deoxy modification.

88. The TREM of any one of claims 85-87, wherein the non-naturally occurring modification is a 2′OMe modification.

89. The TREM of any one of claims 85-88, wherein the non-naturally occurring modification is a 2′halo (e.g., 2′F or 2′Cl) modification.

90. The TREM of any one of claims 85-89, wherein the non-naturally occurring modification is a 2′MOE modification.

91. The TREM of any one of claims 85-90, wherein the non-naturally occurring modification is a 2′-deoxy modification.

92. The TREM of any one of claims 85-91, wherein the non-naturally occurring modification is present in the internucleotide region (e.g., a backbone modification).

93. The TREM of claim 92 wherein the non-naturally occurring modification is a phosphorothioate modification.

94. The TREM of any one of claims 85-93, wherein the TREM has a sequence selected from a sequence provided in FIG. 2.

95. The TREM of any one of claims 85-94, wherein the TREM is a TREM provided in FIG. 2.

96. The TREM of any one of claims 85-95, wherein the TREM comprises a TREM having at least 70%, 75%, 80%, 85%, 90%, 95%, or 99% sequence identity with a TREM provided in FIG. 2.

97. The TREM of any one of claims 85-96, wherein the TREM comprises a sequence that differs by no more than 1, 2, 3, 4, 5, 6, 7, 8, 9, or 10 nucleotides from a TREM provided in FIG. 2.

98. The TREM of any one of claims 85-97, wherein the TREM comprises 1, 2, 3, 4, 5, 6, 7, 8, 9, or 10 additional non-naturally occurring modifications compared with a TREM provided in FIG. 2 (e.g., 2′-ribose modifications or an internucleotide modification, e.g., 2′OMe, 2′-halo, 2′-MOE, 2′-deoxy, or phosphorothiorate modifications).

99. The TREM of any one of claims 85-98, wherein the TREM is selected from TREM NO. 1-500, 501-1000, 1001-1500, 1501-2000, 2001-2500, 2501-3000, 3001-3500, 3501-4000, 4001-4500, 4501-5000, 5001-5500, 5501-6000, 6001-6500, 6501-7000, 7001-7500, 7501-8000, 8001-8500, 8501-9000, and 9001-9136 in FIG. 2.

100. The TREM of any one of claims 85-99, wherein the non-naturally occurring modification is present at a nucleotide position which corresponds to one or more of nucleotides 49-65 of SEQ ID NO: 622.

101. The TREM of any one of claims 85-100, wherein:
(i) the non-naturally occurring modification is present at a nucleotide position which corresponds to one or more of nucleotides 49-65 of SEQ ID NO: 622; and/or
(ii) the TREM differs from the nucleotide sequence of SEQ ID NO: 622 by no more than 1, 2, 3, 4, 5, 6, 7, 8, 9, or 10 nucleotides nucleotides.

102. The TREM of any one of claims 85-101, wherein:
(i) the non-naturally occurring modification is present at a nucleotide position which corresponds to one or more of nucleotides 49-65 of SEQ ID NO: 622; and/or
(ii) the TREM comprises the nucleotide sequence of SEQ ID NO: 622.

103. The TREM of any one of claims 85-101, wherein the TREM comprises the nucleotide sequence of SEQ ID NO: 623.

104. The TREM of any one of claims 85-101, wherein the TREM comprises the nucleotide sequence of SEQ ID NO: 624.

105. The TREM of any one of claims 85-104, wherein the non-naturally occurring modification is present at a nucleotide position which corresponds to one or more of nucleotides 10-25, according to the CtNS.

106. A tRNA effector molecule (TREM) comprising a sequence of Formula (I):
[L1]-[ASt Domain1]-[L2]-[DH Domain]-[L3]-[ACH Domain]-[VL Domain]-[TH Domain]-[L4]-[ASt Domain2] (I),
wherein:
independently, [L1] and [VL Domain], are optional; and
a nucleotide within [L4] or [ASt Domain 2] comprises a nucleotide having a non-naturally occurring modification.

107. The TREM of claim 106, wherein the non-naturally occurring modification is present on the 2′-position of a nucleotide sugar or within the internucleotide region (e.g., a backbone modification).

108. The TREM of any one of claims 106-107, wherein the non-naturally occurring modification is selected from a 2′-O-methyl (2-OMe), 2′-halo (e.g., 2′F or 2′Cl), 2′-O-methoxyethyl (2′MOE), or 2′deoxy modification.

109. The TREM of any one of claims 106-108, wherein the non-naturally occurring modification is a 2′OMe modification.

110. The TREM of any one of claims 106-109, wherein the non-naturally occurring modification is a 2′halo (e.g., 2′F or 2′Cl) modification.

111. The TREM of any one of claims 106-110, wherein the non-naturally occurring modification is a 2′MOE modification.

112. The TREM of any one of claims 106-111, wherein the non-naturally occurring modification is a 2′-deoxy modification.

113. The TREM of any one of claims 106-112, wherein the non-naturally occurring modification is present in the internucleotide region (e.g., a backbone modification).

114. The TREM of claim 113, wherein the non-naturally occurring modification is a phosphorothioate modification.

115. The TREM of any one of claims 106-114, wherein the TREM has a sequence selected from a sequence provided in FIG. 2.

116. The TREM of any one of claims 106-115, wherein the TREM is a TREM provided in FIG. 2.

117. The TREM of any one of claims 106-116, wherein the TREM comprises a TREM having at least 70%, 75%, 80%, 85%, 90%, 95%, or 99% sequence identity with a TREM provided in FIG. 2.

118. The TREM of any one of claims 106-117, wherein the TREM comprises a sequence that differs by no more than 1, 2, 3, 4, 5, 6, 7, 8, 9, or 10 nucleotides from a TREM provided in FIG. 2.

119. The TREM of any one of claims 106-118, wherein the TREM comprises 1, 2, 3, 4, 5, 6, 7, 8, 9, or 10 additional non-naturally occurring modifications compared with a TREM provided in FIG. 2 (e.g., 2′-ribose modifications or an internucleotide modification, e.g., 2′OMe, 2′-halo, 2′-MOE, 2′-deoxy, or phosphorothiorate modifications).

120. The TREM of any one of claims 106-119, wherein the TREM is selected from TREM NO. 1-100, 101-200, 201-300, 301-400, 401-500, 501-600, 601-700, 701-800, 801-900, 901-1000, 1001-1100, 1101-1200, 1201-1300, 1301-1400, 1401-1500, 1501-1600, 1601-1700, 1701-1800, 1801-1900, 1901-2000, 2001-2100, 2101-2200, 2201-2300, 2301-2400, 2401-2500, 2501-2600, and 2601-2663 in FIG. 2.

121. The TREM of any one of claims 106-120, wherein the non-naturally occurring modification is present at a nucleotide position which corresponds to one or more of nucleotides 66-76 of SEQ ID NO: 622.

122. The TREM of any one of claims 106-121, wherein:
(i) the non-naturally occurring modification is present at a nucleotide position which corresponds to one or more of nucleotides 66-76 of SEQ ID NO: 622; and/or
(ii) the TREM differs from the nucleotide sequence of SEQ ID NO: 622 by no more than 1, 2, 3, 4, 5, 6, 7, 8, 9, or 10 nucleotides nucleotides.

123. The TREM of any one of claims 106-122, wherein:
(i) the non-naturally occurring modification is present at a nucleotide position which corresponds to one or more of nucleotides 66-76 of SEQ ID NO: 622; and/or
(ii) the TREM comprises the nucleotide sequence of SEQ ID NO: 622.

124. The TREM of any one of claims 106-122, wherein the TREM comprises the nucleotide sequence of SEQ ID NO: 623.

125. The TREM of any one of claims 106-122, wherein the TREM comprises the nucleotide sequence of SEQ ID NO: 624.

126. The TREM of any one of claims 106-125, wherein the non-naturally occurring modification is present at a nucleotide position which corresponds to one or more of nucleotides 66-76, according to the CtNS.

127. A TREM (e.g., a TREM provided in FIG. 2), according to Design Guidance 1 described herein.

128. A TREM (e.g., a TREM provided in FIG. 2), according to Design Guidance 2 described herein.

129. A TREM (e.g., a TREM provided in FIG. 2), according to Design Guidance 3 described herein.

130. A TREM (e.g., a TREM provided in FIG. 2), according to Design Guidance 4 described herein.

131. A TREM (e.g., a TREM provided in FIG. 2), according to Design Guidance 5 described herein.

132. A TREM (e.g., a TREM provided in FIG. 2), according to Design Guidance 6 described herein.

133. A TREM (e.g., a TREM provided in FIG. 2), according to Design Guidance 1 and 2 described herein.

134. A TREM (e.g., a TREM provided in FIG. 2), according to Design Guidance 1 and 3 described herein.

135. A TREM (e.g., a TREM provided in FIG. 2), according to Design Guidance 1 and 4 described herein.

136. A TREM (e.g., a TREM provided in FIG. 2), according to Design Guidance 1 and 5 described herein.

137. A TREM (e.g., a TREM provided in FIG. 2), according to Design Guidance 1 and 6 described herein.

138. A TREM (e.g., a TREM provided in FIG. 2), according to Design Guidance 2 and 3 described herein.

139. A TREM (e.g., a TREM provided in FIG. 2), according to Design Guidance 2 and 4 described herein.

140. A TREM (e.g., a TREM provided in FIG. 2), according to Design Guidance 2 and 5 described herein.

141. A TREM (e.g., a TREM provided in FIG. 2), according to Design Guidance 2 and 6 described herein.

142. A TREM (e.g., a TREM provided in FIG. 2), according to Design Guidance 3 and 4 described herein.

143. A TREM (e.g., a TREM provided in FIG. 2), according to Design Guidance 3 and 5 described herein.

144. A TREM (e.g., a TREM provided in FIG. 2), according to Design Guidance 3 and 6 described herein.

145. A TREM (e.g., a TREM provided in FIG. 2), according to Design Guidance 4 and 5 described herein.

146. A TREM (e.g., a TREM provided in FIG. 2), according to Design Guidance 4 and 6 described herein.

147. A TREM (e.g., a TREM provided in FIG. 2), according to Design Guidance 5 and 6 described herein.

148. A TREM (e.g., a TREM provided in FIG. 2), according to any three of Design Guidances 1, 2, 3, 4, 5, and 6 described herein.

149. A TREM (e.g., a TREM provided in FIG. 2), according to any four of Design Guidances 1, 2, 3, 4, 5, and 6 described herein.

150. A TREM (e.g., a TREM provided in FIG. 2), according to any five of Design Guidances 1, 2, 3, 4, 5, and 6 described herein.

151. A TREM (e.g., a TREM provided in FIG. 2), according to all of Design Guidances 1, 2, 3, 4, 5, and 6 described herein.

152. A pharmaceutical composition comprising a TREM of any one of the preceding claims.

153. The pharmaceutical composition of claim 152, further comprising a pharmaceutically acceptable component, e.g., an excipient.

154. A lipid nanoparticle formulation comprising a TREM of any one of claims 1-153.

155. A lipid nanoparticle formulation comprising a pharmaceutical composition of claim 154.

156. A method of treating a subject having a disease or disorder associated with a PTC comprising administering to the subject a TREM, TREM core fragment, or TREM fragment described herein (e.g., a TREM of any one of claims 1-151), thereby treating the subject having the disease or disorder.

157. The method of claim 156, wherein the disease or disorder associated with a PTC comprises Hemophilia B, Fabry disease, Usher syndrome, or CLN2 disease.
